(19) United States
(12) Patent Application Publication
(10) Pub. No.: US 2024/0360151 A1
(43) Pub. Date: Oct. 31, 2024

(54) PYRIDAZINE COMPOUNDS, THEIR PREPARATION, AND THEIR THERAPEUTIC USES

(71) Applicant: SANOFI, Paris (FR)

(72) Inventors: Jean-Philippe LETALLEC, Paris (FR); Franck Marguet, Paris (FR); Fréderic Petit, Paris (FR); Baptiste Ronan, Paris (FR); Corinne Terrier, Paris (FR)

(21) Appl. No.: 18/602,519

(22) Filed: Mar. 12, 2024

(30) Foreign Application Priority Data
Mar. 14, 2023 (EP) 23305345.3
Oct. 6, 2023 (EP) 23306724.8

(51) Int. Cl. C07D 498/10; A61K 31/501; A61K 31/5377; A61K 31/538; A61K 31/5383; A61K 31/541; A61K 31/553; C07D 401/04; C07D 403/04; C07D 413/04; C07D 417/04; C07D 471/04; C07D 471/10; C07D 491/107; C07D 498/04

(52) U.S. Cl. CPC C07D 498/10; A61K 31/501; A61K 31/5377; A61K 31/538; A61K 31/5383; A61K 31/541; A61K 31/553; C07D 401/04; C07D 403/04; C07D 413/04; C07D 417/04; C07D 471/04; C07D 471/10; C07D 491/107; C07D 498/04

(57) ABSTRACT

The present invention relates to a compound of formula (I) wherein n is 1 or 2, R1 is halogen, (C₁-C₄)alkyl, halo(C₁-C₄)alkyl, (C₁-C₄)alkoxy, halo(C₁-C₄)alkoxy, ethynyl, propargyl, or (C₃-C₆)cycloalkyl, or two R1 form a cyclopentane ring fused to the phenol; R2 and R3 represent H, cyano, ethynyl, propargyl, (C₁-C₄)alkyl, hydroxy(C₁-C₄)alkyl or a halo(C₁-C₄)alkyl, or R2 and R3 form together with the atoms connecting them a (C₅-C₆)carbocyclic ring fused to the pyridazine ring; R4 and R5 form together with N to which they are attached an optionally substituted 3-7 membered monocyclic heterocycloalkyl ring, 8-11 membered bicyclic heterocycloalkyl ring or 7-12 membered bicyclic heterocyclic spiro ring. The present invention also relates to a medicament and a pharmaceutical composition comprising said compound of formula (I), as well as their therapeutic uses, in particular as inhibitor of NOD-like receptor protein 3 inflammasome for treating for example Parkinson's disease or frontotemporal Dementia.

### CROSS-REFERENCE TO RELATED APPLICATION

This application claims priority to European Patent Application No. 23305345.3, filed Mar. 14, 2023, and priority to European Patent Application No. 23306724.8, filed Oct. 6, 2023, the disclosures of each of which are hereby incorporated herein by reference in their entirety.

### FIELD OF THE DISCLOSURE

Disclosed herein are pyridazine compounds, their preparation, a medicament and a pharmaceutical composition comprising said pyridazine compounds, their use as a medicament and more particularly their use as inhibitor of NOD-like receptor protein 3 (NLRP3) inflammasome pathway, and more particularly their use in the prevention and/or in the treatment of Parkinson's disease, frontotemporal Dementia, Multiple System Atrophy, Alzheimer's disease, Multiple Sclerosis, Amyotrophic Lateral Sclerosis or brain injury.

### BACKGROUND OF THE DISCLOSURE

The NOD-like receptor (NLR) family, pyrin domain-containing protein 3 (NLRP3) or NACHT, LRR and PYD domains-containing protein 3 (NALP3), is a cytosolic sensor of diverse pathogen- and host-derived molecules. Upon activation, NLRP3 oligomerizes and recruits an adaptor protein called apoptosis-associated speck like protein (ASC). ASC then polymerizes to form a large aggregate known as ASC speck. In turn, polymerized ASC interacts with the cysteine protease caspase-1 to form a complex termed the inflammasome. This multicomplex protein forms a platform for the binding, dimerization, and activation of the caspase-1 protease. Caspase-1 then cleaves the precursor forms of the pro-inflammatory cytokines IL1β and IL18 (termed pro-IL1β and pro-IL18) and thereby activates adapted inflammatory responses. However, this pathway was shown to be associated with various inflammation associated processes and diseases, including:
Neurodegenerative diseases such as Parkinson's Disease (PD), Multiple System Atrophy (MSA), Alzheimer's Disease (AD), Frontotemporal Dementia (FTD), Multiple Sclerosis (MS), Amyotrophic Lateral Sclerosis (ALS) and Brain injury (Guan Y & Han F. Front. Integr., Neurosci. 14:37, 2020);
Inflammatory diseases including Muckle-Wells Autoinflammatory Disorder (Agostini et al., 2004), cryopyrin-associated periodic syndrome (CAPS) (Mortimer et al., Nature Immunol. 2016, 17(10), 1176-1188); sickle cell disease; systemic lupus erythematosus (SLE); liver related diseases, viral hepatitis, non-alcoholic steatohepatitis (NASH), alcoholic steatohepatitis, and alcoholic liver disease (Petrasek et al., J. Clin. Invest. 2012, 122, 3476-89), and inflammatory arthritis related disorders, such as gout, pseudogout (chondrocalcinosis), osteoarthritis (Ridker et al., N. Engl. J. Med. 2017, 377, 1119-31), and rheumatoid arthritis (Mathews et al., Ann. Rheum. Dis. 2014, 73, 1202-10), acute or chronic arthropathy, and kidney related diseases such as hyperoxaluria (Knaufet et al., Kidney Int. 2013, 84, 895-901), lupus nephritis, hypertensive nephropathy (Krishnan et al., Br. J. Pharmacol. 2016, 173, 752-10 65), hemodialysis related inflammation and diabetic nephropathy (Shahzad et al., Kidney Int. 2015, 87, 74-84);
Obesity & insulin resistance (Rheinheimer J. et al., Metabolism Clin & Experimental 74: 1-9, 2017); Pancreatitis (Fu Q. et al., BioMed Research International Volume 2018, Article ID 12949512018); Myocarditis (Toldo S et al, Int J Cardiol 2014);
Eye diseases, where the NLRP3 inflammasome has been shown to contribute to diabetic retinopathy (Perrone L. et al., J. Cell. Physiol. 221: 262-272, 2009), acute glaucoma (Chi W. et al. National Academy Science 111: 11181-11186, 2014), age-related macular degeneration (Tseng W. A. et al., Investigative Ophthal & Visual Science 54: 11-120, 2013), Behcet's syndrome and dry eye disease (Zheng Q. et al., Experimental Eye Research 134: 133-140, 2015);
Metabolic, cardiac, skin disorder & cancer, e.g., diabetic cardiomyopathy (Luo B. et al., PLoS ONE 9(8): e104771, 2014); Kawasaki disease (Jia et al. Cell Death and Disease 10:778; 2019; Anzai F. et al., J. Molecular & Cell Cardiology 138: 185-196, 2020); cardiovascular metabolic disorders, atherosclerosis, type I and type II diabetes and related complications, peripheral artery disease (PAD), acute heart failure and hypertension (Ridker et al., N. Engl. J. Med. 2017, 377, 1119-31; wound healing and scar formation; inflammatory skin diseases (Sweeney et al., Br. J. Dermatol. 2015, 173, 1361), asthma, sarcoidosis, age-related macular degeneration; cancer related diseases, e.g., myeloproliferative neoplasms, leukemias, myelodysplastic syndromes (MDS), myelofibrosis, lung cancer, colon cancer (Ridker et al., Lancet 2017, 390, 1833-42); and
SARS-Cov-2: NLRP3 inflammasome is key player in antiviral responses (Zhao C. and Zhao W. 11, 211: 2020; Freeman & Swartz, Frontiers in Immunol. 11, 1518, 2020).

Inhibitors of NLRP3 are potential treatments for these conditions with unmet clinical needs.

Therefore, there is a need for inhibitors of the NLRP3 inflammasome pathway to provide new or alternative treatments.

### SUMMARY OF THE DISCLOSURE

Provided herein is a compound of formula (I) or a pharmaceutically acceptable salt thereof:

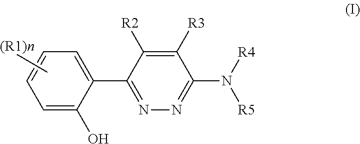

wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

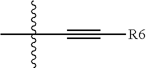

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

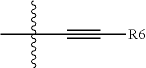

in which R6 is a hydrogen atom or a methyl group and the symbol [symbol] represents the attachment site between the carbon atom and the pyridazine ring;
or R2 and R3 which are on adjacent carbon atoms form together with the atoms connecting them a (C₅-C₆)carbocyclic ring which is fused to the pyridazine ring;
R4 and R5 form together with the nitrogen atom to which they are attached:
   a 3 to 7 membered monocyclic heterocycloalkyl ring comprising one nitrogen atom and optionally one additional heteroatom selected from a nitrogen atom, an oxygen atom and a sulfur atom and comprising 2 to 6 carbon atoms,
   a 8 to 11 membered bicyclic heterocycloalkyl ring comprising one nitrogen atom and optionally one to three additional heteroatoms independently selected from a nitrogen atom, an oxygen atom and a sulfur atom and comprising 4 to 10 carbon atoms, or
   a 7 to 12 membered bicyclic heterocyclic spiro ring comprising one nitrogen atom and optionally one to three additional heteroatoms selected from a nitrogen atom, an oxygen atom and a sulfur atom and comprising 3 to 11 carbon atoms, said monocyclic heterocycloalkyl ring, bicyclic heterocycloalkyl ring and bicyclic heterocyclic spiro ring being unsubstituted or substituted by one to two substituents independently selected from a (C₁-C₄)alkyl-group, a NH₂ group, a mono(C₁-C₄)alkylamino- group, a di(C₁-C₄)alkylamino- group, a NH₂—(C₁-C₄)alkyl-group, a mono(C₁-C₄)alkylamino-(C₁-C₄)alkyl-group, a di(C₁-C₄)alkylamino-(C₁-C₄)alkyl-group, NH₂—(C₃-C₆)cycloalkyl-group, a OH—(C₃-C₆)cycloalkyl-group, a mono(C₁-C₄)alkylamino-(C₃-C₆)cycloalkyl-group, a di(C₁-C₄)alkylamino-(C₃-C₆)cycloalkyl-group, a (C₂-C₅)acylamino-(C₁-C₄)alkyl-group, a (C₂-C₅)acylamino- group, a (C₂-C₅)acyl group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl-group, a mono(C₁-C₄)alkylamino-C(═O)—(C₁-C₄)alkyl-group, a di(C₁-C₄)alkylamino-C(═O)—(C₁-C₄)alkyl-group or a —CO₂H group.

Herein provided are also:
a medicament comprising said compound of formula (I) as defined in the present disclosure or a pharmaceutically acceptable salt thereof,
a pharmaceutical composition comprising said compound of formula (I) as defined in the present disclosure or a pharmaceutically acceptable salt thereof and at least one pharmaceutically acceptable excipient,
said compound of formula (I) according to the present disclosure or a pharmaceutically acceptable salt thereof, for use as a medicine,
said compound of formula (I) according to the present disclosure or a pharmaceutically acceptable salt thereof, for use as inhibitor of NOD-like receptor protein 3 (NLRP3) inflammasome,
said compound of formula (I) according to the present disclosure or a pharmaceutically acceptable salt thereof, for use in the prevention and/or in the treatment of Parkinson's disease, frontotemporal Dementia, Multiple System Atrophy, Alzheimer's disease, Multiple Sclerosis, Amyotrophic Lateral Sclerosis or brain injury.

### Definitions

In the context of the present disclosure, the terms below have the following definitions unless otherwise mentioned throughout the instant specification:
a halogen atom: a fluorine (F), a chlorine (Cl), a bromine (Br) or an iodine (I) atom, and for example a fluorine or a chlorine or an iodine atom;
a hydroxyl group: a “—OH” group;
an oxo group: a “=O” group;
a cyano group: a “—C≡N” group or a CN group;
a —(Cₓ-C_{y})alkyl group: a linear or branched saturated hydrocarbon-based aliphatic group comprising from x to y carbon atoms, for example from 1 to 4 carbon atoms, By way of examples, mention may be made of, but not limited to: methyl, ethyl, propyl, n-propyl, isopropyl, butyl, isobutyl, sec-butyl, tert-butyl groups, and the like;
a —(Cₓ-C_{y})alkoxy group: an —O-alkyl group where the alkyl group is as previously defined. For example, a —(C₁-C₄)alkoxy group. By way of examples, mention may be made of, but not limited to: methoxy, ethoxy, propoxy, isopropoxy, linear, secondary or tertiary butoxy, isobutoxy groups, and the like;
a halo(Cₓ-C_{y})alkyl-group: an alkyl group as previously defined in which one or more hydrogen atoms have been replaced by one or more different or identical halogen atom as previously defined. By way of examples, mention may be made of, but not limited to: trifluoromethyl, fluoromethyl, difluoromethyl, chloromethyl, dichloromethyl, iodomethyl, diiodomethyl, triiodomethyl, diiodofluoromethyl, bromomethyl, tribromomethyl, trichloromethyl, chlorofluoromethyl, fluoroethyl, difluoroethyl, trifluoroethyl, tetrafluoroethyl, chloroethyl, dichloroethyl, trichloroethyl, tetrachloroethyl, iodopropyl, diiodopropyl, bromopropyl, dibromopropyl, tetraiodopropyl, pentaiodopropyl, fluoropropyl, difluoropropyl, trifluoropropyl, tetrafluoropropyl, chloropropyl, dichloropropyl, trichloropropyl, tetrachloropropyl, pentachloropropyl, bromobutyl, dibromobutyl, tetraiodobutyl, pentaiodobutyl, fluorobutyl, difluorobutyl, trifluorobutyl, tetrafluorobutyl, chlorobutyl, dichlorobutyl, trichlorobutyl, tetrachlorobutyl, pentachlorobutyl, hexachlorobutyl, perfluoroethyl, perfluoropropyl, perfluorobutyl, perbromoethyl, perbromoethyl, perbromopropyl, perbromobutyl, periodoethyl, periodoethyl, periodopropyl, periodobutyl, perchloroethyl, perchloroethyl, perchloropropyl, perchlorobutyl groups, and the like;
a halo(Cₓ-C_{y})alkoxy- group: a radical —O-alkyl group in which the alkyl group is as previously defined, said alkyl group being substituted by one or more identical or different halogen atoms as previously defined. By way of examples, mention may be made of, but not limited to: trifluoromethoxy, fluoromethoxy, difluoromethoxy, chlorofluoromethoxy, chloromethoxy, dichloromethoxy, iodomethoxy, diiodomethoxy, triiodomethoxy, bromomethoxy, tribromomethoxy, trichloromethoxy, dichlorobromomethoxy, fluoroethoxy, difluoroethoxy, trifluoroethoxy, tetrafluoroethoxy, chloroethoxy, dichloroethoxy, trichloroethoxy, tetrachloroethoxy, iodopropoxy, diiodopropoxy, bromopropoxy, dibromopropoxy, tetraiodopropoxy, pentaiodopropoxy, fluoropropoxy, difluoropropoxy, trifluoropropoxy, tetrafluoropropoxy, chloropropoxy, dichloropropoxy, trichloropropoxy, tetrachloropropoxy, pentachloropropoxy, bromobutoxy, dibromobutoxy, tetraiodobutoxy, pentaiodobutoxy, fluorobutoxy, difluorobutoxy, trifluorobutoxy, tetrafluorobutoxy, chlorobutoxy, dichlorobutoxy, trichlorobutoxy, tetrachlorobutoxy, pentachlorobutyoxy, hexachlorobutoxy, perfluoroethoxy, perfluoropropoxy, perfluorobutoxy, perbromoethoxy, perbromoethoxy, perbromopropoxy, perbromobutoxy, periodoethoxy, periodoethoxy, periodopropoxy, periodobutoxy, perchloroethoxy, perchloroethoxy, perchloropropoxy, perchlorobutoxy groups, and the like;
a hydroxy(Cₓ-C_{y})alkyl-group: a alkyl group as previously defined in which one or more hydrogen atom have been replaced by one or more hydroxy group as previously defined. By way of examples, mention may be made of, but not limited to: OH—CH₂—, OH—C(CH₃)₂—, CH₃—CH(OH)—, CH₂(OH)—CH(OH)—, CH₃—CH(OH)—CH₂—, CH₃—CH(OH)—CH(OH)—, (CH₃)₂—C(OH)—CH₂—, (CH₃)₂—C(OH)—CH(OH)—, (CH₃)₃—C—CHOH— groups, and the like;
a SH—(C₁-C₄)alkyl-group: a alkyl group as previously defined in which one or more hydrogen atom have been replaced by one or more SH groups. By way of examples, mention may be made of, but not limited to: SH—CH₂—, SH—C(CH₃)₂—, CH₃—CH(SH)—, CH₂(SH)—CH(SH)—, CH₃—CH(SH)—CH₂—, (CH₃)₂—C(SH)—CH₂—, (CH₃)₂—C(SH)—CH(SH)—, (CH₃)₃—C—CH(SH)— groups, and the like;
a CN—(C₁-C₄)alkyl-group: a alkyl group as previously defined in which one or more hydrogen atom have been replaced by one or more CN groups. By way of examples, mention may be made of, but not limited to: CN—CH₂—, CN—C(CH₃)₂—, CH₃—CH(CN)—, CH₂(CN)—CH(CN)—, CH₃—CH(CN)—CH₂—, (CH₃)₂—C(CN)—CH₂—, (CH₃)₂—C(CN)—CH(CN)—, (CH₃)₃—C—CH(CN)— groups, and the like
a NH₂—(C₁-C₄)alkyl-group: a alkyl group as previously defined in which one or more hydrogen atom have been replaced by one or more NH₂ groups. By way of examples, mention may be made of, but not limited to: NH₂—CH₂—, NH₂—C(CH₃)₂—, CH₃—CH(NH₂)—, CH₂(NH₂)—CH(NH₂)—, CH₃—CH(NH₂)—CH₂—, (CH₃)₂—C(NH₂)—CH₂—, (CH₃)₂—C(NH₂)—CH(NH₂)—, (CH₃)₃—C—CH(NH₂)— groups, and the like;
a mono(C₁-C₄)alkylamino- group: an amino group which is substituted by one alkyl group as previously defined. By way of examples, mention may be made of, but not limited to: CH₃—NH—, CH₃—CH₂—NH—, CH₃—CH₂—CH₂—NH—, CH₃—CH₂—CH₂—CH₂—NH—, (CH₃)₂—CH—CH₂—NH—, (CH₃)₃—C—NH—, CH₃—CH₂—CH(CH₃)—NH— groups, and the like;
a di(C₁-C₄)alkylamino- group: an amino group which is substituted by two alkyl groups as previously defined. By way of examples, mention may be made of, but not limited to: (CH₃)₂N—, (CH₃—CH₂)₂N—, (CH₃)(CH₃—CH₂)N—, (CH₃—CH₂—CH₂)₂N—, (CH₃—CH₂—CH₂—CH₂)₂N—, ((CH₃)₂—CH—CH₂)(CH₃)N—, ((CH₃)₃—C)(CH₃CH₂)N—, (CH₃—CH₂—CH(CH₃))(CH₃—CH₂—CH₂)N— groups, and the like;
a (C₂-C₅)acyl- group: a (C₁-C₄)alkyl group as previously defined linked to a —C(═O)— group. By way of examples, mention may be made of, but not limited to: CH₃—C(═O)—, CH₃—CH₂—C(═O)—, CH₃—CH₂—CH₂—C(═O)—, CH₃—CH₂—CH₂—CH₂—C(═O)—, (CH₃)₂—CH—CH₂—C(═O)—, (CH₃)₃—C—C(═O)—, CH₃—CH₂—CH(CH₃)—C(═O)-groups, and the like;
a (C₂-C₅)acylamino- group: an amino group —NH— which is substituted by one acyl group comprising from 2 to 5 carbon atoms, the acyl group being a (C₁-C₄)alkyl group as previously defined linked to a —C(═O)— group which is itself linked to the amino group. By way of examples, mention may be made of, but not limited to: CH₃—C(═O)—NH—, CH₃—CH₂—C(═O)—NH—, CH₃—CH₂—CH₂—C(═O)—NH—, CH₃—CH₂—CH₂—CH₂—C(═O)—NH—, (CH₃)₂—CH—CH₂—C(═O)—NH—, (CH₃)₃—C—C(═O)—NH—, CH₃—CH₂—CH(CH₃)—C(═O)—NH— groups, and the like;
a (C₃-C₆)cycloalkyl-group: a cyclic alkyl group comprising, unless otherwise mentioned, from 3 to 6 carbon atoms, saturated or partially unsaturated and unsubstituted or substituted. By way of examples, mention may be made of, but not limited to: cyclopropyl, cyclobutyl, cyclopentyl, cyclobutenyl, cyclopentenyl, cyclohexenyl, cyclohexyl, and the like;
a OH—(C₃-C₆)cycloalkyl-group: a cyclic alkyl group as defined above in which one or more hydrogen atom have been replaced by one or more hydroxyl groups. By way of examples, mention may be made of, but not limited to: hydroxycyclopropyl, hydroxycyclobutyl, hydroxycyclopentyl, hydroxycyclobutenyl, hydroxycyclopentenyl, hydroxycyclohexenyl, hydroxycyclohexyl, dihydroxycyclopropyl, dihydroxycyclobutyl, trihydroxycyclopentyl, dihydroxycyclobutenyl, trihydroxycyclopentenyl, dihydroxycyclohexenyl, tetrahydroxycyclohexyl and the like;
a NH₂—(C₃-C₆)cycloalkyl-group: a cyclic alkyl group as defined above in which one or more hydrogen atom have been replaced by one or more NH₂ groups. By way of examples, mention may be made of, but not limited to: aminocyclopropyl, aminocyclobutyl, aminocyclopentyl, aminocyclobutenyl, aminocyclopentenyl, aminocyclohexenyl, aminocyclohexyl, diaminocyclopropyl, diaminocyclobutyl, triaminocyclopentyl, diaminocyclobutenyl, triaminocyclopentenyl, diaminocyclohexenyl, tetraaminocyclohexyl and the like;
a (C₅-C₆)carbocyclic ring which is fused to the pyridazine ring: a saturated or partially saturated ring comprising 5 or 6 carbon atoms which is fused to the pyridazine ring. Such carbocyclic ring may be saturated or partially saturated and unsubstituted or substituted. In other terms, the (C₅-C₆)carbocyclic ring is not an aromatic ring and the bicyclic structure formed by the carbocyclic ring and the pyridazine ring comprises three or four double bonds. By way of examples, mention may be made of, but not limited to a cyclopentyl fused to the pyridazine ring, a cyclohexyl fused to the pyridazine ring, so as to form for example:

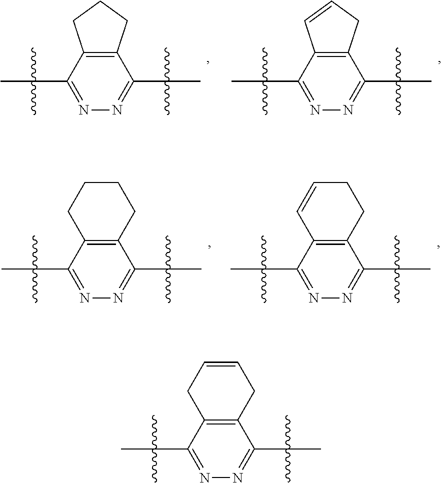 [SMILES: *c1nnc(*)c2c1CCC2 ; *c1nnc(*)c2c1C=CC2 ; *c1nnc(*)c2c1CCCC2 ; *c1nnc(*)c2c1C=CCC2 ; *c1nnc(*)c2c1CC=CC2]

rings, and the like;
a cyclopentane ring which is fused to the phenol group: By way of examples, mention may be made of the following groups:

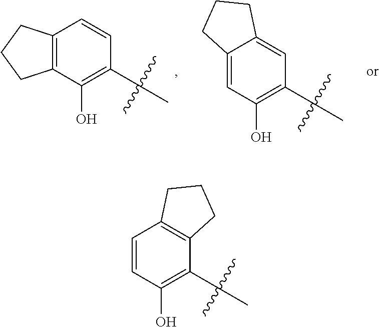 [SMILES: *c1ccc2c(c1O)CCC2 ; *c1cc2c(cc1O)CCC2 ; *c1c(O)ccc2c1CCC2]

in which the symbol [symbol] represents the attachment site between the carbon atom of the phenol ring and the carbon atom of the pyridazine ring. In other words, a cyclopentane ring which is fused to the phenol group as defined in the present disclosure means an indane ring substituted by one hydroxy group, said indane being linked to the pyridazine ring via a single bond in ortho position of the hydroxy group;
a 3 to 7 membered monocyclic heterocycloalkyl ring (also named (C₂-C₆)heterocycloalkyl group): a monocyclic alkyl group comprising, unless otherwise mentioned, from 2 to 6 carbon atoms and comprising one nitrogen atom and optionally one additional heteroatom selected from a nitrogen atom, an oxygen atom and a sulfur atom. Such heterocycloalkyl group may be saturated or partially saturated and unsubstituted or substituted. By way of examples of monocyclic heterocycloalkyl groups, mention may be made of, but not limited to: azepane, piperazine, morpholino, pyrrolidine, tetrahydropyrane, thietane dioxide, piperidine, thiolane, thiolane oxide, thiolane dioxide, dihydrofurane, tetrahydrofurane, azetidine, oxetane, thietane, 2H-pyrrole, 1H-, 2H- or 3H-pyrroline, tetrahydrothiophene, oxadiazole and for example 1,3,4-oxadiazole or 1,3,5-oxadioazole, thiadiazole and for example 1,3,4-thiadiazole, isoxazoline, 2- or 3-pyrazoline, pyrroline, pyrazolidine, imidazoline, imidazolidine, thiazolidine, isooxazoline, isoxazolidine, dioxalane, oxathiazole, oxathiadiazole, dioxazole, thiomorpholino, hexahydropyrimidine groups, and the like;
a 8 to 11 membered bicyclic heterocycloalkyl ring (also named (C₄-C₁₀)heterocycloalkyl group): a bicyclic alkyl group comprising, unless otherwise mentioned, from 4 to 10 carbon atoms and comprising one nitrogen atom and optionally one to three additional heteroatoms selected from a nitrogen atom, an oxygen atom and a sulfur atom. Such heterocycloalkyl group may be saturated or partially saturated, and unsubstituted or substituted, and bridged or not bridged. By way of examples of bicyclic heterocycloalkyl groups, mention may be made of, but not limited to: a dihydropyrazolooxazinyl group such as a dihydro-2H-pyrazolo[4,3-b][1,4]oxazinyl group for instance a 5,6-dihydro-2H-pyrazolo[4,3-b][1,4]oxazinyl group; a hexahydropyridooxazinyl group or a octahydropyridooxazinyl group such as a hexahydro-2H-pyrido[3,4-b][1,4]oxazinyl group, a hexahydro-2H-pyrido[4,3-b][1,4]oxazinyl group, or a octahydropyrido[4,3-b][1,4]oxazinyl group for instance a 3,4a,5,7,8,8a-hexahydro-2H-pyrido[3,4-b][1,4]oxazinyl group, a 3,4a,5,7,8,8a-hexahydro-2H-pyrido[4,3-b][1,4]oxazinyl group or a 2,3,4a,5,6,7,8,8a-octahydropyrido[4,3-b][1,4]oxazinyl group; a dihydropyridooxazinyl group such as a dihydropyrido[3,4-b][1,4]oxazinyl group or dihydropyrido[4,3-b][1,4]oxazinyl ring, for instance a 2,3-dihydropyrido[3,4-b][1,4]oxazinyl group or a 2,3-dihydropyrido[4,3-b][1,4]oxazinyl group; a hexahydropyranooxazinyl group or a octahydropyranooxazinyl group such as hexahydro-2H-pyrano[3,4-b][1,4]oxazinyl group for instance a 3,4a,5,7,8,8a-hexahydro-2H-pyrano[3,4-b][1,4]oxazinyl group, or a hexahydro-2H-pyrano[4,3-b][1,4]oxazinyl group for instance a 3,4a,5,7,8,8a-hexahydro-2H-pyrano[4,3-b][1,4]oxazinyl group; an isoindoline group; an indoline group; a decahydroisoquinoline group; a decahydroquinoline group; a tetrahydroquinoline group; a dihydroquinoline group or a tetrahydroquinoline group such as a dihydro-2H-quinolinyl group for instance a 3,4-dihydro-2H-quinolinyl group; a dihydroisoquinoline group; an azabicyclooctanyl group such as an azabicyclo[3.2.1]octanyl group for instance a 6-azabicyclo[3.2.1]octanyl group; an oxaazabicyclooctanyl group such as an oxaazabicyclo[3.2.1]octanyl group for instance a 3-oxa-8-azabicyclo[3.2.1]octanyl group; a diazabicyclononanyl group for instance a diazabicyclo[3.2.2]nonanyl group such as a 3,6-diazabicyclo[3.2.2]nonanyl group; a hexahydropyrrolooxazinyl group or a octahydropyrrolooxazinyl group such as a hexahydro-2H-pyrrolo[3,4-b][1,4]oxazinyl group for instance a 2,3,4a,5,7,7a-hexahydro-2H-pyrrolo[3,4-b][1,4]oxazinyl group, a 3,4,4a,5,7,7a-hexahydro-2H-pyrrolo[3,4-b][1,4]oxazinyl group, a 3,4a,5,6,7,7a-hexahydro-2H-pyrrolo[3,4-b][1,4]oxazinyl group or a 2,3,4a,5,7,7a-hexahydropyrrolo[3,4-b][1,4]oxazinyl group; a dihydrobenzoxazine group such as a 2,3-dihydro-1,4-benzoxazine group; a octahydropyridooxazinyl group such as a octahydropyrido[4,3-b][1,4]oxazinyl group for instance a 2,3,4a,5,6,7,8,8a-octahydropyrido[4,3-b][1,4]oxazinyl group; a hexahydrofuropyrrolyl ring such as a hexahydrofuro[3,4-c]pyrrolyl ring for instance a 1,3,3a,4,6,6a-hexahydrofuro[3,4-c]pyrrolyl ring; a hexahydropyrrolopyrrolyl ring such as a hexahydropyrrolo[3,4-b]pyrrolyl ring or a hexahydro-2H-pyrrolo[3,4-b]pyrrolyl ring for instance a 2,3,3a,4,6,6a-hexahydropyrrolo[3,4-b]pyrrolyl ring or a 3,3a,4,5,6,6a-hexahydro-2H-pyrrolo[3,4-b]pyrrolyl ring, a hexahydropyrrolo[3,2-b]pyrrolyl ring or a hexahydro-1H-pyrrolo[3,2-b]pyrrolyl ring for instance a 2,3,3a,5,6,6a-hexahydro-1H-pyrrolo[3,2-b]pyrrolyl ring; a hexahydropyrrolopyridyl ring or a octahydropyrrolopyridyl ring such as a hexahydropyrrolo[3,4-c]pyridyl ring for instance a 2,3,3a,6,7,7a-hexahydro-1H-pyrrolo[3,4-c]pyridyl ring, a hexahydropyrrolo[2,3-c]pyridyl ring for instance a 3,3a,4,5,7,7a-hexahydro-2H-pyrrolo[2,3-c]pyridyl ring, or a 2,3,3a,4,5,6,7,7a-octahydropyrrolo[2,3-c]pyridinyl ring; a hexahydrocyclopentaoxazinyl group such as a hexahydrocyclopenta[b][1,4]oxazinyl group for instance a 3,4a,5,6,7,7a-hexahydro-2H-cyclopenta[b][1,4]oxazinyl group; a dihydronaphthyridinyl ring, atetrahydronaphthyridinyl ring, a hexahydronaphthyridinyl ring or a octahydronaphthyridinyl ring such as a dihydro-2H-naphthyridinyl ring for instance a 3,4-dihydro-2H-1,6-naphthyridinyl ring, a hexahydro-2H-1,7-naphthyridinyl ring for instance a 3,4,4a,5,6,8a-hexahydro-2H-1,7-naphthyridinyl ring, a octahydro-1,5-naphthyridinyl ring for instance a 2,3,4,4a,6,7,8,8a-octahydro-1,5-naphthyridinyl ring, or a octahydro-1,7-naphthyridinyl ring for instance a 2,3,4,4a,5,6,7,8a-octahydro-1,7-naphthyridinyl ring or a 2,3,4,4a,5,6,8,8a-octahydro-1,7-naphthyridinyl ring; and the like;
a 7 to 12 membered bicyclic heterocyclic spiro ring: a bicyclic heterocyclic ring system in which the two rings have only one common atom, in particular one common carbon atom, said bicyclic heterocyclic ring system comprises one nitrogen atom and optionally one to three additional heteroatoms selected from a nitrogen atom, an oxygen atom and a sulfur atom and comprises 3 to 11 carbon atoms. Such bicyclic heterocyclic spiro ring system may be saturated or partially saturated and unsubstituted or substituted. By way of examples of bicyclic heterocyclic spiro ring systems, mention may be made of, but not limited to: an azaspirononanyl ring such as an azaspiro[3.5]nonanyl ring, for instance a 7-azaspiro[3.5]nonanyl ring; an azaspirooctanyl ring such as an azaspiro[3.4]octanyl ring, for instance a 6-azaspiro[3.4]octanyl ring or a 2-azaspiro[3.4]octanyl ring; an azaspiroheptanyl ring such as an azaspiro[3.3]heptanyl ring, for instance a 2-azaspiro[3.3]heptanyl ring; a diazaspirononanyl ring such as a diazaspiro[3.5]nonanyl ring, for instance a 2,7-diazaspiro[3.5]nonanyl ring; a diazaspiroheptanyl ring such as a diazaspiro[3.3]heptanyl ring, for instance a 1,6-diazaspiro[3.3]heptanyl ring; an oxaazaspirononanyl ring such as a oxaazaspiro[3.5]nonanyl ring, for instance a 2-oxa-7-azaspiro[3.5]nonanyl ring; an oxaazaspiroheptanyl ring such as an oxaazaspiro[3.3]heptanyl ring, for instance a 2-oxa-6-azaspiro[3.3]heptanyl ring; a dithiaazaspirononanyl ring such as a dithiaazaspiro[4.4]nonanyl ring for instance a 1,4-dithia-7-azaspiro[4.4]nonanyl ring; an oxadiazaspirononanyl ring such as an oxadiazaspiro[3.5]nonanyl ring for instance a 8-oxa-2,5-diazaspiro[3.5]nonanyl ring; an oxadiazaspirodecanyl ring such as an oxadiazaspiro[4.5]decanyl ring for instance a 2-oxa-6,9-diazaspiro[4.5]decanyl ring, a 6-oxa-2,9-diazaspiro[4.5]decanyl ring or a 9-oxa-2,6-diazaspiro[4.5]decanyl ring; an oxadiazaspiroundecanyl ring such as an oxadiazaspiro[5.5]undecanyl ring for instance a 4-oxa-1,8-diazaspiro[5.5]undecanyl ring; a diazaspirodecanyl ring such as a diazaspiro[4.5]decanyl ring for instance a 1,9-diazaspiro[4.5]decanyl ring; a dioxadiazaspirododecanyl ring such as a dioxadiazaspiro[5.6]dodecanyl ring for instance a 1,11-dioxa-4,8-diazaspiro[5.6]dodecanyl ring; and the like;
“optionally substituted” means “unsubstituted or “substituted with”.
R and S indicate the stereochemistry of the pseudo-asymmetric carbon atoms, according to the IUPAC rules.
in each radical or group as defined in the present disclosure, each hydrogen atom can be replaced by a deuterium atom, which forms part of the present disclosure.

### DETAILED DESCRIPTION OF THE DISCLOSURE

As mentioned above, herein is provided a compound of formula (I) or a pharmaceutically acceptable salt thereof:

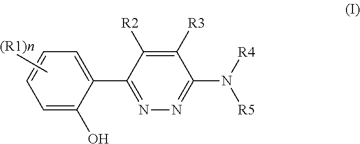

wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

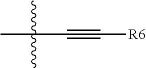

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

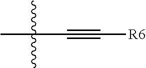

in which R6 is a hydrogen atom or a methyl group and the symbol [symbol] represents the attachment site between the carbon atom and the pyridazine ring;
or R2 and R3 which are on adjacent carbon atoms form together with the atoms connecting them a (C₅-C₆)carbocyclic ring which is fused to the pyridazine ring; R4 and R5 form together with the nitrogen atom to which they are attached:
   a 3 to 7 membered monocyclic heterocycloalkyl ring comprising one nitrogen atom and optionally one additional heteroatom selected from a nitrogen atom, an oxygen atom and a sulfur atom and comprising 2 to 6 carbon atoms,
   a 8 to 11 membered bicyclic heterocycloalkyl ring comprising one nitrogen atom and optionally one to three additional heteroatoms independently selected from a nitrogen atom, an oxygen atom and a sulfur atom and comprising 4 to 10 carbon atoms, or
   a 7 to 12 membered bicyclic heterocyclic spiro ring comprising one nitrogen atom and optionally one to three additional heteroatoms selected from a nitrogen atom, an oxygen atom and a sulfur atom and comprising 3 to 11 carbon atoms, said monocyclic heterocycloalkyl ring, bicyclic heterocycloalkyl ring and bicyclic heterocyclic spiro ring being unsubstituted or substituted by one to two substituents independently selected from a (C₁-C₄)alkyl-group, a NH₂ group, a mono(C₁-C₄)alkylamino- group, a di(C₁-C₄)alkylamino- group, a NH₂—(C₁-C₄)alkyl-group, a mono(C₁-C₄)alkylamino-(C₁-C₄)alkyl-group, a di(C₁-C₄)alkylamino-(C₁-C₄)alkyl-group, NH₂—(C₃-C₆)cycloalkyl-group, a OH—(C₃-C₆)cycloalkyl-group, a mono(C₁-C₄)alkylamino-(C₃-C₆)cycloalkyl-group, a di(C₁-C₄)alkylamino-(C₃-C₆)cycloalkyl-group, a (C₂-C₅)acylamino-(C₁-C₄)alkyl-group, a (C₂-C₅)acylamino- group, a (C₂-C₅)acyl group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl-group, a mono(C₁-C₄)alkylamino-C(═O)—(C₁-C₄)alkyl-group, a di(C₁-C₄)alkylamino-C(═O)—(C₁-C₄)alkyl-group or a —CO₂H group.

Also provided is a compound of formula (I) or a pharmaceutically acceptable salt thereof, wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

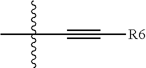

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

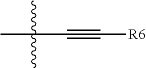

in which R6 is a hydrogen atom or a methyl group and the symbol [symbol] represents the attachment site between the carbon atom and the pyridazine ring;
R4 and R5 form together with the nitrogen atom to which they are attached:
   a 3 to 7 membered monocyclic heterocycloalkyl ring comprising one nitrogen atom and optionally one additional heteroatom selected from a nitrogen atom, an oxygen atom and a sulfur atom and comprising 2 to 6 carbon atoms,
   a 8 to 11 membered bicyclic heterocycloalkyl ring comprising one nitrogen atom and optionally one to three additional heteroatoms independently selected from a nitrogen atom, an oxygen atom and a sulfur atom and comprising 4 to 10 carbon atoms, or
   a 7 to 12 membered bicyclic heterocyclic spiro ring comprising one nitrogen atom and optionally one to three additional heteroatoms selected from a nitrogen atom, an oxygen atom and a sulfur atom and comprising 3 to 11 carbon atoms, said monocyclic heterocycloalkyl ring, bicyclic heterocycloalkyl ring and bicyclic heterocyclic spiro ring being unsubstituted or substituted by one to two substituents independently selected from a (C₁-C₄)alkyl-group, a NH₂ group, a mono(C₁-C₄)alkylamino- group, a di(C₁-C₄)alkylamino- group, a NH₂—(C₁-C₄)alkyl-group, a mono(C₁-C₄)alkylamino-(C₁-C₄)alkyl-group, a di(C₁-C₄)alkylamino-(C₁-C₄)alkyl-group, NH₂—(C₃-C₆)cycloalkyl-group, a OH—(C₃-C₆)cycloalkyl-group, a mono(C₁-C₄)alkylamino-(C₃-C₆)cycloalkyl-group, a di(C₁-C₄)alkylamino-(C₃-C₆)cycloalkyl-group, a (C₂-C₅)acylamino-(C₁-C₄)alkyl-group, a (C₂-C₅)acylamino- group, a (C₂-C₅)acyl group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl-group, a mono(C₁—C₄)alkylamino-C(═O)—(C₁-C₄)alkyl-group, a di(C₁-C₄)alkylamino-C(═O)—(C₁-C₄)alkyl-group or a —CO₂H group.

Also provided is a compound of formula (I) or a pharmaceutically acceptable salt thereof, wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

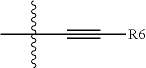

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 which are on adjacent carbon atoms form together with the atoms connecting them a (C₅-C₆)carbocyclic ring which is fused to the pyridazine ring;
R4 and R5 form together with the nitrogen atom to which they are attached:
   a 3 to 7 membered monocyclic heterocycloalkyl ring comprising one nitrogen atom and optionally one additional heteroatom selected from a nitrogen atom, an oxygen atom and a sulfur atom and comprising 2 to 6 carbon atoms,
   a 8 to 11 membered bicyclic heterocycloalkyl ring comprising one nitrogen atom and optionally one to three additional heteroatoms independently selected from a nitrogen atom, an oxygen atom and a sulfur atom and comprising 4 to 10 carbon atoms, or
   a 7 to 12 membered bicyclic heterocyclic spiro ring comprising one nitrogen atom and optionally one to three additional heteroatoms selected from a nitrogen atom, an oxygen atom and a sulfur atom and comprising 3 to 11 carbon atoms, said monocyclic heterocycloalkyl ring, bicyclic heterocycloalkyl ring and bicyclic heterocyclic spiro ring being unsubstituted or substituted by one to two substituents independently selected from a (C₁-C₄)alkyl-group, a NH₂ group, a mono(C₁-C₄)alkylamino- group, a di(C₁-C₄)alkylamino- group, a NH₂—(C₁-C₄)alkyl-group, a mono(C₁-C₄)alkylamino-(C₁-C₄)alkyl-group, a di(C₁—C₄)alkylamino-(C₁-C₄)alkyl-group, NH₂—(C₃-C₆)cycloalkyl-group, a OH—(C₃-C₆)cycloalkyl-group, a mono(C₁-C₄)alkylamino-(C₃-C₆)cycloalkyl-group, a di(C₁-C₄)alkylamino-(C₃-C₆)cycloalkyl-group, a (C₂-C₅)acylamino-(C₁-C₄)alkyl-group, a (C₂-C₅)acylamino- group, a (C₂-C₅)acyl group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl-group, a mono(C₁-C₄)alkylamino-C(═O)—(C₁-C₄)alkyl-group, a di(C₁-C₄)alkylamino-C(═O)—(C₁-C₄)alkyl-group or a —CO₂H group.

The compounds of formula (I) may comprise one or more asymmetric carbons. They may exist in the form of enantiomers or diastereoisomers. The compounds of formula (I) may also exist in the form of cis or trans stereoisomers. These stereoisomers, enantiomers and diastereoisomers, and also mixtures thereof, including racemic mixtures, form part of the disclosure.

The compounds of formula (I) may be present as well under tautomer forms.

The compounds of formula (I) may exist in the form of bases, acids, zwitterion or of addition salts with acids or bases. Hence, herein are provided compounds of formula (I) or pharmaceutically acceptable salts thereof.

These salts may be prepared with pharmaceutically acceptable acids or bases, although the salts of other acids or bases useful, for example, for purifying or isolating the compounds of formula (I) are also provided.

Among suitable salts of the compounds of formula (I), trifluoroacetate, and formate may be cited.

Among the compounds of formula (I) that are subject-matter of the disclosure, a group of compounds is composed of the compounds for which n is 1.

Among the compounds of formula (I) that are subject-matter of the disclosure, a group of compounds is composed of the compounds for which n is 2.

Among the compounds of formula (I) that are subject-matter of the disclosure, a group of compounds is composed of the compounds for which R1 represent independently of each other a halogen atom, a (C₁-C₄)alkyl group, a (C₁-C₄)alkoxy group, a halo(C₁-C₄)alkoxy group, a (C₃-C₆)cycloalkyl group, a group of formula

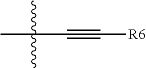

in which R6 is a hydrogen atom or a methyl group and the symbol [symbol] represents the attachment site between the carbon atom and the phenyl ring, or a halo(C₁-C₄)alkyl group.

Among the compounds of formula (I) that are subject-matter of the disclosure, a group of compounds is composed of the compounds for which R1 represent independently of each other a fluorine atom (F), a chlorine atom (Cl), a methyl group, an ethyl group, an isopropyl group, a methoxy group, a trifluoromethoxy group, a cyclopropyl group, a —CHF₂ group, an ethynyl group or a trifluoromethyl group.

Among the compounds of formula (I) that are subject-matter of the disclosure, a group of compounds is composed of the compounds for which when n is 1, R1 is in ortho, meta or para position on the phenyl with respect to pyridazine ring.

Among the compounds of formula (I) that are subject-matter of the disclosure, a group of compounds is composed of the compounds for which when n is 1, R1 is in ortho or para position on the phenyl with respect to pyridazine ring.

Among the compounds of formula (I) that are subject-matter of the disclosure, a group of compounds is composed of the compounds for which when n is 2, the two R1 are in ortho and in para position on the phenyl with respect to pyridazine ring.

Among the compounds of formula (I) that are subject-matter of the disclosure, a group of compounds is composed of the compounds for which when n is 2, the two R1 are in ortho and in meta position on the phenyl with respect to pyridazine ring.

Among the compounds of formula (I) that are subject-matter of the disclosure, a group of compounds is composed of the compounds for which when n is 2, the two R1 which are on adjacent carbon atoms form together with the atoms connecting them a cyclopentane ring which is fused to the phenol group, in particular these two R1 represent a group of formula

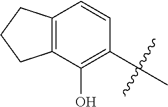 [SMILES: *c1ccc2c(c1O)CCC2]

in which the symbol [symbol] represents the attachment site between the carbon atom of the phenol ring and the carbon atom of the pyridazine ring.

Among the compounds of formula (I) that are subject-matter of the disclosure, a group of compounds is composed of the compounds for which R2 and R3 represent independently of each other a hydrogen atom, a cyano group, a halo(C₁-C₄)alkyl group, a hydroxy(C₁-C₄)alkyl group or a (C₁-C₄)alkyl group.

Among the compounds of formula (I) that are subject-matter of the disclosure, a group of compounds is composed of the compounds for which R2 and R3 represent independently of each other a hydrogen atom, a cyano group, a —CHF₂ group, a trifluoromethyl group, a —CH₂OH group, an ethyl group, an isopropyl group or a methyl group.

Among the compounds of formula (I) that are subject-matter of the disclosure, a group of compounds is composed of the compounds for which R2 represents a hydrogen atom, a halo(C₁-C₄)alkyl group, a hydroxy(C₁-C₄)alkyl group or a (C₁-C₄)alkyl group.

Among the compounds of formula (I) that are subject-matter of the disclosure, a group of compounds is composed of the compounds for which R2 represents a hydrogen atom, a —CHF₂ group, a trifluoromethyl group, a —CH₂OH group, an ethyl group, an isopropyl group or a methyl group.

Among the compounds of formula (I) that are subject-matter of the disclosure, a group of compounds is composed of the compounds for which R3 represents a hydrogen atom, a cyano group, a halo(C₁-C₄)alkyl group, a hydroxy(C₁-C₄)alkyl group or a (C₁-C₄)alkyl group.

Among the compounds of formula (I) that are subject-matter of the disclosure, a group of compounds is composed of the compounds for which R3 represents a hydrogen atom, a cyano group, a —CHF₂ group, a —CH₂OH group, or a methyl group.

Among the compounds of formula (I) that are subject-matter of the disclosure, a group of compounds is composed of the compounds for which R2 and R3 which are on adjacent carbon atoms form together with the atoms connecting them a (C₅-C₆)carbocyclic ring which is fused to the pyridazine ring.

Among the compounds of formula (I) that are subject-matter of the disclosure, a group of compounds is composed of the compounds for which R2 and R3 which are on adjacent carbon atoms form together with the atoms connecting them a cyclopentyl ring fused to the pyridazine ring which has the following formula:

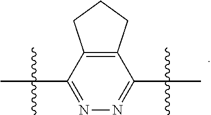 [SMILES: *c1nnc(*)c2c1CCC2]

Among the compounds of formula (I) that are subject-matter of the disclosure, a group of compounds is composed of the compounds for which R4 and R5 form together with the nitrogen atom to which they are attached:
a 4 to 7 membered monocyclic heterocycloalkyl ring comprising one nitrogen atom and optionally one additional heteroatom selected from a nitrogen atom, an oxygen atom and a sulfur atom and comprising 3 to 6 carbon atoms,
a 8 to 10 membered bicyclic heterocycloalkyl ring comprising one nitrogen atom and optionally one or two additional heteroatoms independently selected from a nitrogen atom, an oxygen atom and a sulfur atom and comprising 6, 7, 8 or 9 carbon atoms, or
a 7 to 10 or 12 membered bicyclic heterocyclic spiro ring comprising one nitrogen atom and optionally one to three additional heteroatoms selected from a nitrogen atom, an oxygen atom and a sulfur atom and comprising 5 to 8 carbon atoms, said monocyclic heterocycloalkyl ring, bicyclic heterocycloalkyl ring and bicyclic heterocyclic spiro ring being unsubstituted or substituted by one to two substituents independently selected from a (C₁-C₄)alkyl-group, a NH₂ group, a mono(C₁-C₄)alkylamino-group, a di(C₁-C₄)alkylamino- group, a NH₂—(C₁-C₄)alkyl-group, a mono(C₁-C₄)alkylamino-(C₁-C₄)alkyl-group, a di(C₁-C₄)alkylamino-(C₁-C₄)alkyl-group, NH₂—(C₃-C₆)cycloalkyl-group, a (C₂-C₅)acylamino-(C₁-C₄)alkyl-group, a (C₂-C₅)acylamino- group, a (C₂-C₅)acyl- group, a hydroxy-(C₁-C₄)alkyl-group, a SH—(C₁-C₄)alkyl-group; a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)— group, a di(C₁-C₄)alkylamino-C(═O)-group, a NH₂—C(═O)—(C₁-C₄)alkyl-group, a mono(C₁-C₄)alkylamino-C(═O)—(C₁-C₄)alkyl-group, or a di(C₁-C₄)alkylamino-C(═O)—(C₁-C₄)alkyl-group.

Among the compounds of formula (I) that are subject-matter of the disclosure, a group of compounds is composed of the compounds for which R4 and R5 form together with the nitrogen atom to which they are attached:
a 4 to 7 membered monocyclic heterocycloalkyl ring comprising one nitrogen atom and optionally one additional heteroatom selected from a nitrogen atom, an oxygen atom and a sulfur atom and comprising 3 to 6 carbon atoms selected from the group consisting of an azepanyl ring, a morpholino ring, a thiomorpholino ring, a piperidinyl ring, a pyrrolidinyl ring, an isoxazolidinyl ring, a piperazinyl ring, a hexahydropyrimidinyl ring, and an azetidinyl ring;
a 8 to 10 membered bicyclic heterocycloalkyl ring comprising one nitrogen atom and optionally one or two additional heteroatoms independently selected from a nitrogen atom, an oxygen atom and a sulfur atom and comprising 6, 7, 8 or 9 carbon atoms selected from the group consisting of an indolinyl ring; a hexahydropyridooxazinyl ring, a hexahydropyranooxazinyl ring, a octahydropyranooxazinyl ring, a dihydropyridooxazinyl ring, a dihydroquinolinyl group, a tetrahydroquinolinyl group, an azabicyclooctanyl group, an oxaazabicyclooctanyl group; a diazabicyclononanyl group, a hexahydropyrrolooxazinyl group; a octahydropyrrolooxazinyl group; a dihydrobenzoxazinyl group; a octahydropyridooxazinyl group; a hexahydrofuropyrrolyl ring; a hexahydropyrrolopyrrolyl ring; a hexahydropyrrolopyridinyl ring; a octahydropyrrolopyridinyl ring; a dihydropyrazolooxazolinyl ring, a hexahydrocyclopentaoxazinyl group, a hexahydronaphthyridinyl ring, a octahydronaphthyridinyl ring, a tetrahydronaphthyridinyl ring and a dihydronaphthyridinyl ring; or
a 7 to 10 or 12 membered bicyclic heterocyclic spiro ring comprising one nitrogen atom and optionally one to three additional heteroatoms selected from a nitrogen atom, an oxygen atom and a sulfur atom and comprising 5 to 8 carbon atoms selected from the group consisting of an azaspirononanyl ring, an azaspirooctanyl ring, an azaspiroheptanyl ring, a diazaspirodecanyl ring, a diazaspirononanyl ring, a diazaspiroheptanyl ring, an oxadiazaspirodecanyl ring, an oxadiazaspiroundecanyl ring, an oxadiazaspirononanyl ring, an dioxadiazaspirododecanyl ring, an oxaazaspirononanyl ring, and an oxaazaspiroheptanyl ring,
said monocyclic heterocycloalkyl ring, bicyclic heterocycloalkyl ring and bicyclic heterocyclic spiro ring being unsubstituted or substituted by one to two substituents independently selected from a (C₁-C₄)alkyl-group, a NH₂ group, a mono(C₁-C₄)alkylamino-group, a di(C₁-C₄)alkylamino- group, a mono(C₁-C₄)alkylamino-(C₁-C₄)alkyl-group, a NH₂—(C₁-C₄)alkyl-group, a di(C₁-C₄)alkylamino-(C₁-C₄)alkyl-group, NH₂—(C₃-C₆)cycloalkyl-group, a (C₂-C₅)acylamino-(C₁-C₄)alkyl-group, a (C₂-C₅)acylamino- group, a (C₂-C₅)acyl- group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)— group, a di(C₁-C₄)alkylamino-C(═O)-group, a NH₂—C(═O)—(C₁-C₄)alkyl-group, a mono(C₁-C₄)alkylamino-C(═O)—(C₁-C₄)alkyl-group, or a di(C₁-C₄)alkylamino-C(═O)—(C₁-C₄)alkyl-group.

Among the compounds of formula (I) that are subject-matter of the disclosure, a group of compounds is composed of the compounds for which R4 and R5 form together with the nitrogen atom to which they are attached:
an azepanyl ring unsubstituted or substituted by one OH—CH₂— group;
a morpholino ring unsubstituted or substituted by one or two substituents independently selected from a methyl group, a CH₂F— group, NH₂—CH₂— group, a N(CH₃)₂—CH₂— group, a CH₃—NH—(CH₂)₂— group, a SH—CH₂— group, a CN—CH₂— group, a OH—CH₂-group, a OH-CD2- group, a OH—(CH₂)₂— group, a OH—C(CH₃)₂— group, a CH₃—CH(OH)-group, a CH₃—C(═O)—NH—CH₂— group, a CH₃—C(═O)—NH—CH₂—CH₂— group a CH(CH₃)₂—C(═O)—NH—CH₂— group, a NH₂—C(═O)— group, a NH(CH₃)—C(═O)— group, a N(CH₃)₂—C(═O)-group, a NH₂-cyclopropyl- group, a NH₂—C(═O)—CH₂— group, a NH(CH₃)—C(═O)—CH₂-group, a N(CH₃)₂—C(═O)—CH₂— group, a CH₃—CH₂—C(═O)— group, an oxo group, and a CH₃—O—CH₂— group,
a thiomorpholino ring unsubstituted or substituted by one to two oxo groups;
a piperidinyl ring unsubstituted or substituted by one to two substituents independently selected from a NH₂ group, a NH₂—CH₂— group, a NH(CH₂CH₃)— group, a N(CH₃)₂— group, a NH₂-cyclopropyl- group, a N(CH₃)₂—CH₂— group, a OH—CH₂— group, a CH₃—C(═O)—NH—CH₂— group, a cyano group, a hydroxyl group, an oxo group and a fluorine atom,
a pyrrolidinyl ring unsubstituted or substituted by one to two substituents independently selected from a NH₂— group, a NH₂—CH₂— group, a N(CH₃)₂—CH₂— group, a OH—CH₂— group, a CH₃—O—CH₂— group, a hydroxyl group, and an oxo group,
an isoxazolidinyl ring unsubstituted or substituted by one hydroxyl group,
a piperazinyl ring unsubstituted or substituted by one to two substituents independently selected from a methyl group, an oxo group and a OH—CH₂— group;
a hexahydropyrimidinyl ring substituted by one oxo group;
an azetidinyl ring unsubstituted or substituted by one OH—CH₂— group,
a tetrahydroquinolinyl ring or a dihydroquinolinyl ring such as 3,4-dihydro-2H-quinolinyl ring, unsubstituted or substituted by one hydroxyl group
a hexahydropyridooxazinyl ring such as a hexahydro-2H-pyrido[3,4-b][1,4]oxazinyl ring, for instance a 3,4a,5,7,8,8a-hexahydro-2H-pyrido[3,4-b][1,4]oxazinyl group, or a hexahydro-2H-pyrido[4,3-b][1,4]oxazin-4-yl ring, for instance a 3,4a,5,7,8,8a-hexahydro-2H-pyrido[4,3-b][1,4]oxazinyl ring, unsubstituted or substituted by one cyclobutyl group, one methyl group, one ethyl group, one 2,2,2-trifluoroethyl group, one 2-fluoroethyl group, one 3-fluoropropyl group, one isopropropyl group or one acetyl group;
a hexahydropyranooxazinyl ring such as hexahydro-2H-pyrano[3,4-b][1,4]oxazinyl ring, for instance a 3,4a,5,7,8,8a-hexahydro-2H-pyrano[3,4-b][1,4]oxazinyl group, or such as a hexahydro-2H-pyrano[4,3-b][1,4]oxazinyl ring, for instance a 3,4a,5,7,8,8a-hexahydro-2H-pyrano[4,3-b][1,4]oxazinyl ring, unsubstituted;
a dihydropyridooxazinyl ring such as dihydropyrido[3,4-b][1,4]oxazinyl ring or dihydropyrido[4,3-b][1,4]oxazinyl ring, for instance 2,3-dihydropyrido[3,4-b][1,4]oxazinyl ring or 2,3-dihydropyrido[4,3-b][1,4]oxazinyl ring, unsubstituted;
a dihydropyrazolooxazinyl ring such as dihydro-2H-pyrazolo[4,3-b][1,4]oxazinyl ring, for instance 5,6-dihydro-2H-pyrazolo[4,3-b][1,4]oxazinyl ring, unsubstituted;
a dihydrobenzoxazinyl ring such as dihydro-1,4-benzoxazinyl ring, for instance 2,3-dihydro-1,4-benzoxazinyl ring substituted by one hydroxyl group;
a tetrahydronaphthyridinyl ring or a dihydronaphthyridinyl ring such as 3,4-dihydro-2H-1,6-naphthyridinyl ring substituted by one hydroxyl group;
a hexahydronaphthyridinyl ring such as hexahydro-2H-1,7-naphthyridinyl ring for instance a 3,4,4a,5,6,8a-hexahydro-2H-1,7-naphthyridinyl ring substituted by one methyl group and one oxo group;
a octahydronaphthyridinyl ring such as octahydro-1,7-naphthyridinyl ring or octahydro-1,5-naphthyridinyl ring for instance a 2,3,4,4a,5,6,7,8a-octahydro-1,7-naphthyridinyl ring, a 2,3,4,4a,5,6,8,8a-octahydro-1,7-naphthyridinyl ring, or a 2,3,4,4a,6,7,8,8a-octahydro-1,5-naphthyridinyl ring, substituted by one oxo group, one methyl group, or one acetyl group;
an octahydropyrrolopyridinyl ring or a hexahydropyrrolopyridinyl ring such as hexahydro-1H-pyrrolo[3,4-c]pyridinyl ring, octahydropyrrolo[2,3-c]pyridinyl ring, or hexahydro-2H-pyrrolo[2,3-c]pyridinyl ring, for instance 2,3,3a,6,7,7a-hexahydro-1H-pyrrolo[3,4-c]pyridinyl ring, 2,3,3a,4,5,6,7,7a-octahydropyrrolo[2,3-c]pyridinyl ring or 3,3a,4,5,7,7a-hexahydro-2H-pyrrolo[2,3-c]pyridinyl ring, unsubstituted or substituted by one methyl group, one ethyl group, one acetyl group, one isopropyl group or one oxo group;
an octahydropyrrolooxazinyl ring or a hexahydropyrrolooxazinyl ring such as hexahydro-2H-pyrrolo[3,4-b][1,4]oxazinyl ring, for instance 2,3,4a,5,7,7a-hexahydro-2H-pyrrolo[3,4-b][1,4]oxazinyl ring, 3,4,4a,5,7,7a-hexahydro-2H-pyrrolo[3,4-b][1,4]oxazinyl ring or 3,4a,5,6,7,7a-hexahydro-2H-pyrrolo[3,4-b][1,4]oxazinyl ring, unsubstituted or substituted by one methyl group, one ethyl group or one acetyl group;
a hexahydrocyclopentaoxazinyl ring such as 3,4a,5,6,7,7a-hexahydro-2H-cyclopenta[b][1,4]oxazinyl ring substituted by one hydroxyl group;
an octahydropyridooxazinyl ring such as octahydropyrido[4,3-b][1,4]oxazinyl ring, for instance 2,3,4a,5,6,7,8,8a-octahydropyrido[4,3-b][1,4]oxazinyl ring, unsubstituted;
an indolinyl ring substituted by one hydroxyl group or one —CH₂OH group;
a hexahydropyrrolopyrrolyl ring such as hexahydropyrrolo[3,4-b]pyrrolyl ring, hexahydro-1H-pyrrolo[3,2-b]pyrrolyl ring, or hexahydro-2H-pyrrolo[3,4-b]pyrrolyl ring, for instance 2,3,3a,4,6,6a-hexahydropyrrolo[3,4-b]pyrrolyl ring, 2,3,3a,5,6,6a-hexahydro-1H-pyrrolo[3,2-b]pyrrolyl ring, or a 3,3a,4,5,6,6a-hexahydro-2H-pyrrolo[3,4-b]pyrrolyl ring unsubstituted or substituted by one methyl group, one ethyl group or one acetyl group;
a hexahydrofuropyrrolyl ring such as hexahydrofuro[3,4-c]pyrrolyl ring for instance 1,3,3a,4,6,6a-hexahydrofuro[3,4-c]pyrrolyl ring, unsubstituted;
a azabicyclooctanyl ring such as 6-azabicyclo[3.2.1]octanyl ring substituted by one hydroxyl group;
a diazabicyclononanyl ring such as 3,6-diazabicyclo[3.2.2]nonanyl ring substituted by one methyl group
a oxaazabicyclooctanyl ring such as 3-oxa-8-azabicyclo[3.2.1]octanyl ring unsubstituted;
an azaspirononanyl ring such as an azaspiro[3.5]nonanyl ring, unsubstituted or substituted by one substituent selected from a NH₂ group, a CH₃—C(═O)—NH— group, and a hydroxyl group;
an azaspirooctanyl ring such as an azaspiro[3.4]octanyl ring, unsubstituted or substituted by one hydroxyl group;
an azaspiroheptanyl ring such as an azaspiro[3.3]heptanyl ring, unsubstituted or substituted by one hydroxyl group;
a diazaspirodecanyl ring such as 1,9-diazaspiro[4.5]decanyl ring substituted by one methyl group;
a diazaspirononanyl ring such as a diazaspiro[3.5]nonanyl ring, unsubstituted or substituted by one methyl group;
a diazaspiroheptanyl ring such as a diazaspiro[3.3]heptanyl ring, unsubstituted or substituted by one a methyl group;
an oxadiazaspirononanyl ring such as 8-oxa-2,5-diazaspiro[3.5]nonanyl ring substituted by one methyl group;
an oxadiazaspirodecanyl ring such as 2-oxa-6,9-diazaspiro[4.5]decanyl ring, 9-oxa-2,6-diazaspiro[4.5]decanyl ring, or 6-oxa-2,9-diazaspiro[4.5]decanyl ring unsubstituted or substituted by one ethyl group or one methyl group;
an oxadiazaspiroundecanyl ring such as 4-oxa-1,8-diazaspiro[5.5]undecanyl ring substituted by one methyl group;
an dioxadiazaspirododecanyl ring such as 1,11-dioxa-4,8-diazaspiro[5.6]dodecanyl ring substituted by one ethyl group;
an oxaazaspirononanyl ring such as a oxaazaspiro[3.5]nonanyl ring; and
an oxaazaspiroheptanyl ring such as an oxaazaspiro[3.3]heptanyl ring.

All these sub-groups taken alone or in combination are part of the present disclosure.

According to a particular embodiment, the disclosure relates to a compound of formula (I) or a pharmaceutically acceptable salt thereof:

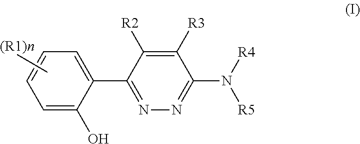

wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

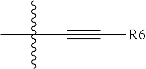

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

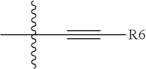

in which R6 is a hydrogen atom or a methyl group and the symbol [symbol] represents the attachment site between the carbon atom and the pyridazine ring; or R2 and R3 which are on adjacent carbon atoms form together with the atoms connecting them a (C₅-C₆)carbocyclic ring which is fused to the pyridazine ring; and R4 and R5 form together with the nitrogen atom to which they are attached:
   a 3 to 7 membered monocyclic heterocycloalkyl ring comprising one nitrogen atom and optionally one additional heteroatom selected from a nitrogen atom, an oxygen atom and a sulfur atom and comprising 2 to 6 carbon atoms,
said monocyclic heterocycloalkyl ring being unsubstituted or substituted by one to two substituents independently selected from a (C₁-C₄)alkyl-group, a NH₂ group, a mono(C₁-C₄)alkylamino- group, a di(C₁-C₄)alkylamino- group, a NH₂—(C₁-C₄)alkyl-group, a mono(C₁-C₄)alkylamino-(C₁-C₄)alkyl-group, a di(C₁-C₄)alkylamino-(C₁-C₄)alkyl-group, NH₂—(C₃-C₆)cycloalkyl-group, a OH—(C₃-C₆)cycloalkyl-group, a mono(C₁-C₄)alkylamino-(C₃-C₆)cycloalkyl-group, a di(C₁-C₄)alkylamino-(C₃-C₆)cycloalkyl-group, a (C₂-C₅)acylamino-(C₁-C₄)alkyl-group, a (C₂-C₅)acylamino- group, a (C₂-C₅)acyl group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl-group, a mono(C₁-C₄)alkylamino-C(═O)—(C₁-C₄)alkyl-group, a di(C₁-C₄)alkylamino-C(═O)—(C₁-C₄)alkyl-group or a —CO₂H group.

According to another particular embodiment, the disclosure relates to a compound of formula (I) or a pharmaceutically acceptable salt thereof, wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

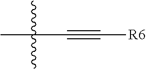

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

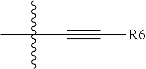

in which R6 is a hydrogen atom or a methyl group and the symbol [symbol] represents the attachment site between the carbon atom and the pyridazine ring; and
R4 and R5 form together with the nitrogen atom to which they are attached:
   a 3 to 7 membered monocyclic heterocycloalkyl ring comprising one nitrogen atom and optionally one additional heteroatom selected from a nitrogen atom, an oxygen atom and a sulfur atom and comprising 2 to 6 carbon atoms,
said monocyclic heterocycloalkyl ring being unsubstituted or substituted by one to two substituents independently selected from a (C₁-C₄)alkyl-group, a NH₂ group, a mono(C₁-C₄)alkylamino- group, a di(C₁-C₄)alkylamino- group, a NH₂—(C₁-C₄)alkyl-group, a mono(C₁-C₄)alkylamino-(C₁-C₄)alkyl-group, a di(C₁-C₄)alkylamino-(C₁-C₄)alkyl-group, NH₂—(C₃-C₆)cycloalkyl-group, a OH—(C₃-C₆)cycloalkyl-group, a mono(C₁-C₄)alkylamino-(C₃-C₆)cycloalkyl-group, a di(C₁-C₄)alkylamino-(C₃-C₆)cycloalkyl-group, a (C₂-C₅)acylamino-(C₁-C₄)alkyl-group, a (C₂-C₅)acylamino- group, a (C₂-C₅)acyl group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl-group, a mono(C₁-C₄)alkylamino-C(═O)—(C₁-C₄)alkyl-group, a di(C₁-C₄)alkylamino-C(═O)—(C₁-C₄)alkyl-group or a —CO₂H group.

According to another particular embodiment, the disclosure relates to a compound of formula (I) or a pharmaceutically acceptable salt thereof, wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

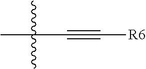

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 which are on adjacent carbon atoms form together with the atoms connecting them a (C₅-C₆)carbocyclic ring which is fused to the pyridazine ring; and
R4 and R5 form together with the nitrogen atom to which they are attached:
   a 3 to 7 membered monocyclic heterocycloalkyl ring comprising one nitrogen atom and optionally one additional heteroatom selected from a nitrogen atom, an oxygen atom and a sulfur atom and comprising 2 to 6 carbon atoms,
said monocyclic heterocycloalkyl ring being unsubstituted or substituted by one to two substituents independently selected from a (C₁-C₄)alkyl-group, a NH₂ group, a mono(C₁-C₄)alkylamino- group, a di(C₁-C₄)alkylamino- group, a NH₂—(C₁-C₄)alkyl-group, a mono(C₁-C₄)alkylamino-(C₁-C₄)alkyl-group, a di(C₁-C₄)alkylamino-(C₁-C₄)alkyl-group, NH₂—(C₃-C₆)cycloalkyl-group, a OH—(C₃-C₆)cycloalkyl-group, a mono(C₁—C₄)alkylamino-(C₃-C₆)cycloalkyl-group, a di(C₁-C₄)alkylamino-(C₃-C₆)cycloalkyl-group, a (C₂-C₅)acylamino-(C₁-C₄)alkyl-group, a (C₂-C₅)acylamino- group, a (C₂-C₅)acyl group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl-group, a mono(C₁-C₄)alkylamino-C(═O)—(C₁-C₄)alkyl-group, a di(C₁-C₄)alkylamino-C(═O)—(C₁-C₄)alkyl-group or a —CO₂H group.

According to another particular embodiment, the disclosure relates to a compound of formula (II) or a pharmaceutically acceptable salt thereof:

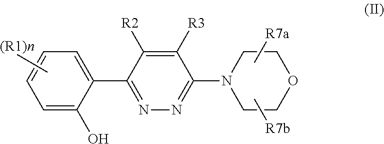

wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

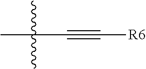

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

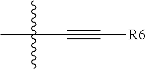

in which R6 is a hydrogen atom or a methyl group and the symbol [symbol] represents the attachment site between the carbon atom and the pyridazine ring;
or R2 and R3 which are on adjacent carbon atoms form together with the atoms connecting them a (C₅-C₆)carbocyclic ring which is fused to the pyridazine ring; and
R7a and R7b represent independently of each other a hydrogen atom, a (C₁-C₄)alkyl-group, a NH₂ group, a mono(C₁-C₄)alkylamino- group, a di(C₁-C₄)alkylamino- group, a NH₂—(C₁-C₄)alkyl-group, a mono(C₁-C₄)alkylamino-(C₁-C₄)alkyl-group, a di(C₁-C₄)alkylamino-(C₁-C₄)alkyl-group, NH₂—(C₃-C₆)cycloalkyl-group, a OH—(C₃-C₆)cycloalkyl-group, a mono(C₁-C₄)alkylamino-(C₃-C₆)cycloalkyl-group, a di(C₁-C₄)alkylamino-(C₃-C₆)cycloalkyl-group, a (C₂-C₅)acylamino-(C₁-C₄)alkyl-group, a (C₂-C₅)acylamino- group, a (C₂-C₅)acyl group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)-group, a di(C₁-C₄)alkylamino-C(═O)— group, a NH₂—C(═O)—(C₁-C₄)alkyl-group, a mono(C₁-C₄)alkylamino-C(═O)—(C₁-C₄)alkyl-group, a di(C₁-C₄)alkylamino-C(═O)—(C₁-C₄)alkyl-group or a —CO₂H group.

According to another particular embodiment, the disclosure relates to a compound of formula (II) or a pharmaceutically acceptable salt thereof:

wherein
n is 1 or 2;
R1 represent independently of each other a halogen atom, a (C₁-C₄)alkyl group, a (C₁-C₄)alkoxy group, a halo(C₁-C₄)alkoxy group, a halo(C₁-C₄)alkyl group, a group of formula

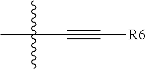

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group or a halo(C₁-C₄)alkyl group;
or R2 and R3 which are on adjacent carbon atoms form together with the atoms connecting them a (C₅-C₆)carbocyclic ring which is fused to the pyridazine ring; and
R7a and R7b represent independently of each other a hydrogen atom, a (C₁-C₄)alkyl-group, a halo-(C₁-C₄)alkyl-group, a NH₂—C(═O)— group, NH₂—(C₁-C₄)alkyl-group, a mono(C₁-C₄)alkylamino-(C₁-C₄)alkyl-group, a di(C₁-C₄)alkylamino-(C₁-C₄)alkyl-group, a (C₂-C₅)acylamino-(C₁-C₄)alkyl-group, a hydroxy-(C₁-C₄)alkyl-group, a SH—(C₁-C₄)alkyl-group, a CN—(C₁-C₄)alkyl-group, a (C₂-C₅)acyl group, a mono(C₁-C₄)alkylamino-C(═O)— group, a di(C₁-C₄)alkylamino-C(═O)— group, a NH₂—(C₃-C₆)cycloalkyl-group, a NH₂—C(═O)—(C₁-C₄)alkyl-group, a mono(C₁-C₄)alkylamino-C(═O)—(C₁-C₄)alkyl-group, a di(C₁-C₄)alkylamino-C(═O)—(C₁-C₄)alkyl-group, an oxo group, or a (C₁-C₄)alkoxy-(C₁-C₄)alkyl-group.

According to another particular embodiment, the disclosure relates to a compound of formula (II) or a pharmaceutically acceptable salt thereof:

wherein
n is 1 or 2;
R1 represent independently of each other a halogen atom, a (C₁-C₄)alkyl group, a (C₁-C₄)alkoxy group, a halo(C₁-C₄)alkoxy group, a halo(C₁-C₄)alkyl group, a group of formula

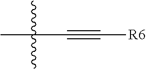

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group or a halo(C₁-C₄)alkyl group;
or R2 and R3 which are on adjacent carbon atoms form together with the atoms connecting them a (C₅-C₆)carbocyclic ring which is fused to the pyridazine ring; R7a represents a hydrogen atom, a (C₁-C₄)alkyl-group, a halo-(C₁-C₄)alkyl-group, a NH₂—C(═O)— group, NH₂—(C₁-C₄)alkyl-group, a mono(C₁-C₄)alkylamino-(C₁-C₄)alkyl-group, a di(C₁-C₄)alkylamino-(C₁-C₄)alkyl-group, a (C₂-C₅)acylamino-(C₁-C₄)alkyl-group, a hydroxy-(C₁-C₄)alkyl-group, a SH—(C₁-C₄)alkyl-group, a CN—(C₁-C₄)alkyl-group, a (C₂-C₅)acyl group, a mono(C₁-C₄)alkylamino-C(═O)— group, a di(C₁-C₄)alkylamino-C(═O)— group, a NH₂—(C₃-C₆)cycloalkyl-group, a NH₂—C(═O)—(C₁-C₄)alkyl-group, a mono(C₁-C₄)alkylamino-C(═O)—(C₁-C₄)alkyl-group, a di(C₁—C₄)alkylamino-C(═O)—(C₁-C₄)alkyl-group, an oxo group or a (C₁-C₄)alkoxy-(C₁-C₄)alkyl-group; and
R7b represents a hydrogen atom, or a hydroxy-(C₁-C₄)alkyl-group.

According to another particular embodiment, the disclosure relates to a compound of formula (II) or a pharmaceutically acceptable salt thereof:

wherein
n is 1 or 2
R1 represent independently of each other a methyl group, an ethyl group, an isopropyl group, an ethynyl group, F, Cl, a methoxy group, a cyclopropyl group, a trifluoromethoxy group or a trifluoromethyl group;
or two R1 which are on adjacent carbon atoms form together with the atoms connecting them a cyclopentane ring which is fused to the phenol group so as to form the following formula

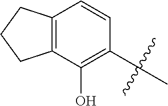 [SMILES: *c1ccc2c(c1O)CCC2]

in which the symbol [symbol] represents the attachment site between the carbon atom of the phenol ring and the carbon atom of the pyridazine ring;
R2 and R3 represent independently of each other a hydrogen atom, a cyano group, a —CHF₂ group, a trifluoromethyl group, a OH—CH₂— group, a methyl group, an ethyl group, or an isopropyl group;
or R2 and R3 which are on adjacent carbon atoms form together with the atoms connecting them a cyclopentyl ring which is fused to the pyridazine ring; and
R7a and R7b represent independently of each other a hydrogen atom, a methyl group, a CH₂F— group, NH₂—CH₂— group, a N(CH₃)₂—CH₂— group, a SH—CH₂— group, a CN—CH₂-group, a CH₃—NH—(CH₂)₂— group, a OH—CH₂— group, a OH-CD₂- group, a OH—(CH₂)₂-group, a OH—C(CH₃)₂— group, a CH₃—CH(OH)— group, a CH₃—C(═O)—NH—CH₂— group, a CH₃—C(═O)—NH—CH₂—CH₂— group a CH(CH₃)₂—C(═O)—NH—CH₂— group, a NH₂—C(═O)-group, a NH(CH₃)—C(═O)— group, a N(CH₃)₂—C(═O)— group, a NH₂-cyclopropyl- group, a NH₂—C(═O)—CH₂— group, a NH(CH₃)—C(═O)—CH₂— group, a N(CH₃)₂—C(═O)—CH₂— group, a CH₃—CH₂—C(═O)— group, an oxo group, or a CH₃—O—CH₂— group.

According to another particular embodiment, the disclosure relates to a compound of formula (II) or a pharmaceutically acceptable salt thereof:

wherein
n is 1 or 2;
R1 represent independently of each other a methyl group, an ethyl group, an isopropyl group, an ethynyl group, F, Cl, a methoxy group, a cyclopropyl group, a trifluoromethoxy group or a trifluoromethyl group;
or two R1 which are on adjacent carbon atoms form together with the atoms connecting them a cyclopentane ring which is fused to the phenol group so as to form the following formula

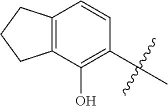 [SMILES: *c1ccc2c(c1O)CCC2]

in which the symbol [symbol] represents the attachment site between the carbon atom of the phenol ring and the carbon atom of the pyridazine ring;
R2 and R3 represent independently of each other a hydrogen atom, a cyano group, a —CHF₂ group, a trifluoromethyl group, a OH—CH₂— group, a methyl group, an ethyl group, or an isopropyl group;
or R2 and R3 which are on adjacent carbon atoms form together with the atoms connecting them a cyclopentyl ring which is fused to the pyridazine ring;
R7a represents a hydrogen atom, a methyl group, a CH₂F— group, NH₂—CH₂— group, a N(CH₃)₂—CH₂— group, a SH—CH₂— group, a CN—CH₂— group, a CH₃—NH—(CH₂)₂— group, a OH—CH₂— group, a OH-CD₂- group, a OH—(CH₂)₂— group, a OH—C(CH₃)₂— group, a CH₃—CH(OH)— group, a CH₃—C(═O)—NH—CH₂— group, a CH₃—C(═O)—NH—CH₂—CH₂— group a CH(CH₃)₂—C(═O)—NH—CH₂— group, a NH₂—C(═O)— group, a NH(CH₃)—C(═O)— group, a N(CH₃)₂—C(═O)— group, a NH₂-cyclopropyl- group, a NH₂—C(═O)—CH₂— group, a NH(CH₃)—C(═O)—CH₂— group, a N(CH₃)₂—C(═O)—CH₂— group, a CH₃—CH₂—C(═O)— group, an oxo group, or a CH₃—O—CH₂— group; and
R7b represents a hydrogen atom or a OH—CH₂— group.

According to another particular embodiment, the disclosure relates to a compound of formula (III) or a pharmaceutically acceptable salt thereof:

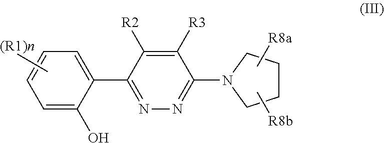

wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

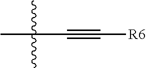

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

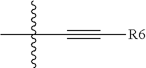

in which R6 is a hydrogen atom or a methyl group and the symbol [symbol] represents the attachment site between the carbon atom and the pyridazine ring;
or R2 and R3 which are on adjacent carbon atoms form together with the atoms connecting them a (C₅-C₆)carbocyclic ring which is fused to the pyridazine ring; and R8a and R8b represent independently of each other a hydrogen atom, a (C₁-C₄)alkyl-group, a NH₂ group, a mono(C₁-C₄)alkylamino- group, a di(C₁-C₄)alkylamino- group, a NH₂—(C₁-C₄)alkyl-group, a mono(C₁-C₄)alkylamino-(C₁-C₄)alkyl-group, a di(C₁-C₄)alkylamino-(C₁-C₄)alkyl-group, NH₂—(C₃-C₆)cycloalkyl-group, a OH—(C₃-C₆)cycloalkyl-group, a mono(C₁-C₄)alkylamino-(C₃-C₆)cycloalkyl-group, a di(C₁-C₄)alkylamino-(C₃-C₆)cycloalkyl-group, a (C₂-C₅)acylamino-(C₁-C₄)alkyl-group, a (C₂-C₅)acylamino- group, a (C₂-C₅)acyl group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)-group, a di(C₁-C₄)alkylamino-C(═O)— group, a NH₂—C(═O)—(C₁-C₄)alkyl-group, a mono(C₁-C₄)alkylamino-C(═O)—(C₁-C₄)alkyl-group, a di(C₁-C₄)alkylamino-C(═O)—(C₁-C₄)alkyl-group or a —CO₂H group.

According to another particular embodiment, the disclosure relates to a compound of formula (III) or a pharmaceutically acceptable salt thereof:
wherein
n is 2;
R1 represent independently of each other a halogen atom, a (C₁-C₄)alkyl group, or a halo(C₁-C₄)alkyl group;
R2 and R3 represent a hydrogen atom, or a halo(C₁-C₄)alkyl group; and
R8a and R8b represent independently of each other a hydrogen atom, a NH₂— group, a NH₂—(C₁-C₄)alkyl-group, a di(C₁-C₄)alkylamino-(C₁-C₄)alkyl-group, a hydroxy-(C₁-C₄)alkyl-group, a (C₁-C₄)alkoxy-(C₁-C₄)alkyl-group, a hydroxyl group, or an oxo group.

According to another particular embodiment, the disclosure relates to a compound of formula (III) or a pharmaceutically acceptable salt thereof:
wherein
n is 2;
R1 represent independently of each other a halogen atom, a (C₁-C₄)alkyl group, or a halo(C₁-C₄)alkyl group;
R2 represents a halo(C₁-C₄)alkyl group or a hydrogen atom;
R3 represents a hydrogen atom;
R8a represents a hydrogen atom, a NH₂— group, a NH₂—(C₁-C₄)alkyl-group, a di(C₁-C₄)alkylamino-(C₁-C₄)alkyl-group, a hydroxy-(C₁-C₄)alkyl-group, a (C₁-C₄)alkoxy-(C₁-C₄)alkyl-group, a hydroxyl group, or an oxo group; and
R8b represents a hydrogen atom, or an oxo group.

According to another particular embodiment, the disclosure relates to a compound of formula (III) or a pharmaceutically acceptable salt thereof:
wherein
n is 2;
R1 represent independently of each other a chlorine atom, a methyl group, or a trifluoromethyl group;
R2 represents a hydrogen atom or a —CHF₂ group;
R3 represents a hydrogen atom; and
R8a and R8b represent independently of each other a hydrogen atom, a NH₂— group, a NH₂—(CH₂)— group, a N(CH₃)₂—(CH₂)— group, a OH—CH₂— group, a CH₃—O—CH₂— group, a hydroxyl group, or an oxo group.

According to another particular embodiment, the disclosure relates to a compound of formula (III) or a pharmaceutically acceptable salt thereof:
wherein
n is 2;
R1 represent independently of each other a chlorine atom, a methyl group, or a trifluoromethyl group;
R2 represents a hydrogen atom or a —CHF₂ group;
R3 represents a hydrogen atom;
R8a represents a hydrogen atom, a NH₂— group, a NH₂—(CH₂)— group, a N(CH₃)₂—(CH₂)— group, a OH—CH₂— group, a CH₃—O—CH₂— group, a hydroxyl group, or an oxo group; and
R8b represents a hydrogen atom, or an oxo group.

According to another particular embodiment, the disclosure relates to a compound of formula (IV) or a pharmaceutically acceptable salt thereof:

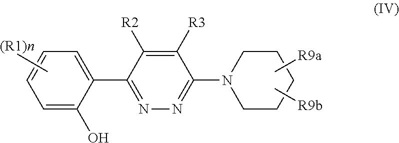

wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

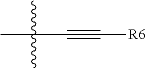

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

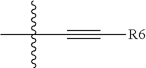

in which R6 is a hydrogen atom or a methyl group and the symbol [symbol] represents the attachment site between the carbon atom and the pyridazine ring;
or R2 and R3 which are on adjacent carbon atoms form together with the atoms connecting them a (C₅-C₆)carbocyclic ring which is fused to the pyridazine ring;
R9a and R9b represent independently of each other a hydrogen atom, a (C₁-C₄)alkyl-group, a NH₂ group, a mono(C₁-C₄)alkylamino- group, a di(C₁-C₄)alkylamino- group, a NH₂—(C₁-C₄)alkyl-group, a mono(C₁-C₄)alkylamino-(C₁-C₄)alkyl-group, a di(C₁-C₄)alkylamino-(C₁-C₄)alkyl-group, NH₂—(C₃-C₆)cycloalkyl-group, a OH—(C₃-C₆)cycloalkyl-group, a mono(C₁-C₄)alkylamino-(C₃-C₆)cycloalkyl-group, a di(C₁-C₄)alkylamino-(C₃-C₆)cycloalkyl-group, a (C₂-C₅)acylamino-(C₁-C₄)alkyl-group, a (C₂-C₅)acylamino- group, a (C₂-C₅)acyl group, a hydroxy-(C₁-C₄)alkyl-group, a SH—(C₁-C₄)alkyl-group, a CN—(C₁-C₄)alkyl-group, a (C₁-C₄)alkoxy-(C₁-C₄)alkyl-group, a hydroxyl group, an oxo group, a (C₃-C₆)cycloalkyl group, a halogen atom, a halo(C₁-C₄)alkyl-group, a cyano group, a NH₂—C(═O)— group, a mono(C₁-C₄)alkylamino-C(═O)-group, a di(C₁-C₄)alkylamino-C(═O)— group, a NH₂—C(═O)—(C₁-C₄)alkyl-group, a mono(C₁-C₄)alkylamino-C(═O)—(C₁-C₄)alkyl-group, a di(C₁-C₄)alkylamino-C(═O)—(C₁-C₄)alkyl-group or a —CO₂H group.

According to another particular embodiment, the disclosure relates to a compound of formula (IV) or a pharmaceutically acceptable salt thereof:
wherein
n is 2;
R1 represent independently of each other a halogen atom, a (C₁-C₄)alkyl group, or a halo(C₁-C₄)alkyl group;
R2 and R3 represent a hydrogen atom; and
R9a and R9b represent independently of each other a hydrogen atom, a NH₂ group, a mono(C₁-C₄)alkylamino- group, a di(C₁-C₄)alkylamino- group, a NH₂—(C₁-C₄)alkyl-group, a di(C₁-C₄)alkylamino-(C₁-C₄)alkyl-group, NH₂—(C₃-C₆)cycloalkyl-group, a (C₂-C₅)acylamino-(C₁-C₄)alkyl-group, a hydroxy-(C₁-C₄)alkyl-group, a hydroxyl group, a cyano group, an oxo group or a halogen atom.

According to another particular embodiment, the disclosure relates to a compound of formula (IV) or a pharmaceutically acceptable salt thereof:
wherein
n is 2;
R1 represent independently of each other a halogen atom, a (C₁-C₄)alkyl group, or a halo(C₁-C₄)alkyl group;
R2 and R3 represent a hydrogen atom;
R9a represents a hydrogen atom, a NH₂ group, a mono(C₁-C₄)alkylamino- group, a di(C₁-C₄)alkylamino- group, a NH₂—(C₁-C₄)alkyl-group, a di(C₁-C₄)alkylamino-(C₁-C₄)alkyl-group, NH₂—(C₃-C₆)cycloalkyl-group, a (C₂-C₅)acylamino-(C₁-C₄)alkyl-group, a hydroxy-(C₁-C₄)alkyl-group, a hydroxyl group, a cyano group, or a halogen atom; and
R9b represents a hydrogen atom, an oxo group, or a halogen atom.

According to another particular embodiment, the disclosure relates to a compound of formula (IV) or a pharmaceutically acceptable salt thereof:
wherein
n is 2;
R1 represent independently of each other a chlorine atom, a methyl group, or a trifluoromethyl group;
R2 and R3 represent a hydrogen atom; and
R9a and R9b represent independently of each other a hydrogen atom, a NH₂ group, a N(CH₃)₂— group, a CH₃—CH₂—NH— group, a NH₂—CH₂— group, a N(CH₃)₂—CH₂— group, a NH₂-cyclopropyl- group, a CH₃—(C═O)—NH—CH₂— group, a OH—CH₂— group, a hydroxyl group, a cyano group, an oxo group or a fluorine atom (F).

According to another particular embodiment, the disclosure relates to a compound of formula (IV) or a pharmaceutically acceptable salt thereof:
wherein
n is 2;
R1 represent independently of each other a chlorine atom, a methyl group, or a trifluoromethyl group;
R2 and R3 represent a hydrogen atom;
R9a represents a hydrogen atom, a NH₂ group, a N(CH₃)₂— group, a CH₃—CH₂—NH— group, a NH₂—CH₂— group, a N(CH₃)₂—CH₂— group, a NH₂-cyclopropyl- group, a CH₃—(C═O)—NH—CH₂— group, a OH—CH₂— group, a hydroxyl group, a cyano group, or a fluorine atom (F); and
R9b represents a hydrogen atom, an oxo group, or a fluorine atom (F).

Among the compounds of formula (I) or a pharmaceutically acceptable salt thereof that are subject-matter of the present disclosure, mention may be made for instance to the following compounds:
(1) 1-[6-(2-hydroxy-4,6-dimethyl-phenyl)pyridazin-3-yl]piperidin-4-ol;
(2) 1-[6-(2-hydroxy-4,6-dimethyl-phenyl)pyridazin-3-yl]piperidine-4-carbonitrile;
(3) 7-[6-(2-hydroxy-4,6-dimethyl-phenyl)pyridazin-3-yl]-7-azaspiro[3.5]nonan-2-ol;
(4) 3,5-dimethyl-2-[6-[rac-(4aS,8aR)-6-cyclobutyl-3,4a,5,7,8,8a-hexahydro-2H-pyrido[3,4-b][1,4]oxazin-1-yl]pyridazin-3-yl]phenol;
(5) 2-[6-[(3R)-3-(hydroxymethyl)morpholin-4-yl]pyridazin-3-yl]-3,5-dimethyl-phenol;
(6) 2-[6-[(3S)-3-(hydroxymethyl)morpholin-4-yl]pyridazin-3-yl]-3,5-dimethyl-phenol;
(7) 2-[6-(2,7-diazaspiro[3.5]nonan-7-yl)pyridazin-3-yl]-3,5-dimethyl-phenol; 2,2,2-trifluoroacetic acid;
(8) 2-[6-[(2S)-2-(hydroxymethyl)morpholin-4-yl]pyridazin-3-yl]-3,5-dimethyl-phenol;
(9) 2-[6-[(2R)-2-(hydroxymethyl)morpholin-4-yl]pyridazin-3-yl]-3,5-dimethyl-phenol;
(10) 2-[6-[(4aR,8aS)-6-cyclobutyl-3,4a,5,7,8,8a-hexahydro-2H-pyrido[4,3-b][1,4]oxazin-4-yl]pyridazin-3-yl]-3,5-dimethyl-phenol Enantiomer 1;
(11) 2-[6-[(4aS,8aR)-6-cyclobutyl-3,4a,5,7,8,8a-hexahydro-2H-pyrido[4,3-b][1,4]oxazin-4-yl]pyridazin-3-yl]-3,5-dimethyl-phenol; formic acid Enantiomer 2;
(12) 2-[6-[(4aR,8aS)-6-cyclobutyl-3,4a,5,7,8,8a-hexahydro-2H-pyrido[4,3-b][1,4]oxazin-4-yl]pyridazin-3-yl]-3-methyl-5-(trifluoromethyl)phenol Enantiomer 1;
(13) 2-[6-[(4aS,8aR)-6-cyclobutyl-3,4a,5,7,8,8a-hexahydro-2H-pyrido[4,3-b][1,4]oxazin-4-yl]pyridazin-3-yl]-3-methyl-5-(trifluoromethyl)phenol Enantiomer 2;
(14) 2-[6-[(2R)-2-(methoxymethyl)pyrrolidin-1-yl]pyridazin-3-yl]-3-methyl-5-(trifluoromethyl)phenol;
(15) 2-[6-[(2S)-2-(methoxymethyl)pyrrolidin-1-yl]pyridazin-3-yl]-3-methyl-5-(trifluoromethyl)phenol;
(16) 2-[6-[(2S)-2-(hydroxymethyl)morpholin-4-yl]pyridazin-3-yl]-3-methyl-5-(trifluoromethyl)phenol;
(17) 2-[6-[3-(1-aminocyclopropyl)-1-piperidyl]pyridazin-3-yl]-3,5-dichloro-phenol;
(18) 2-[6-[(3S)-3-(aminomethyl)-1-piperidyl]pyridazin-3-yl]-3,5-dichloro-phenol;
(19) 2-[6-[4-(aminomethyl)-1-piperidyl]pyridazin-3-yl]-3,5-dichloro-phenol;
(20) 2-[6-[(3S)-3-(hydroxymethyl)pyrrolidin-1-yl]pyridazin-3-yl]-3-methyl-5-(trifluoromethyl)phenol;
(21) 3,5-dichloro-2-[6-[(2S)-2-(hydroxymethyl)morpholin-4-yl]pyridazin-3-yl]phenol;
(22) 2-[6-[(3R)-3-(aminomethyl)-1-piperidyl]pyridazin-3-yl]-3,5-dichloro-phenol;
(23) 2-[6-[(3S)-3-(aminomethyl)pyrrolidin-1-yl]pyridazin-3-yl]-3,5-dichloro-phenol;
(24) 2-[6-[2-[(dimethylamino)methyl]morpholin-4-yl]pyridazin-3-yl]-3-methyl-5-(trifluoromethyl)phenol;
(25) 2-[6-[(3R)-3-(aminomethyl)pyrrolidin-1-yl]pyridazin-3-yl]-3,5-dichloro-phenol;
(26) 2-[6-[2-(1-hydroxy-1-methyl-ethyl)morpholin-4-yl]pyridazin-3-yl]-3-methyl-5-(trifluoromethyl)phenol;
(27) 2-[6-[(2R)-2-[(dimethylamino)methyl]morpholin-4-yl]pyridazin-3-yl]-3-methyl-5-(trifluoromethyl)phenol;
(28) 2-[6-[(3S)-3-(hydroxymethyl)-1-piperidyl]pyridazin-3-yl]-3-methyl-5-(trifluoromethyl)phenol;
(29) 3-methyl-2-[6-(4-methylpiperazin-1-yl)pyridazin-3-yl]-5-(trifluoromethyl)phenol;
(30) 2-[6-[3-(hydroxymethyl)azetidin-1-yl]pyridazin-3-yl]-3-methyl-5-(trifluoromethyl)phenol;
(31) 2-[6-[(3R)-3-amino-1-piperidyl]pyridazin-3-yl]-3,5-dimethyl-phenol; formic acid;
(32) 2-[6-(2-amino-7-azaspiro[3.5]nonan-7-yl)pyridazin-3-yl]-3,5-dichloro-phenol;
(33) 3,5-dichloro-2-[6-[(2R)-2-(methoxymethyl)morpholin-4-yl]pyridazin-3-yl]phenol;
(34) 3,5-dichloro-2-[6-[(3S)-3-(hydroxymethyl)-1-piperidyl]pyridazin-3-yl]phenol;
(35) 3,5-dichloro-2-[6-[(2S)-2-(methoxymethyl)morpholin-4-yl]pyridazin-3-yl]phenol;
(36) 3,5-dichloro-2-[6-[(3R)-3-(hydroxymethyl)-1-piperidyl]pyridazin-3-yl]phenol;
(37) 7-[6-(2,4-dichloro-6-hydroxy-phenyl)pyridazin-3-yl]-7-azaspiro[3.5]nonan-2-ol;
(38) 3,5-dichloro-2-(6-morpholinopyridazin-3-yl)phenol;
(39) 3,5-dichloro-2-[6-[(2S)-2-[(dimethylamino)methyl]morpholin-4-yl]pyridazin-3-yl]phenol;
(40) 3,5-dichloro-2-[6-[(3S)-3-[(dimethylamino)methyl]pyrrolidin-1-yl]pyridazin-3-yl]phenol;
(41) 3,5-dichloro-2-[6-[(3R)-3-[(dimethylamino)methyl]pyrrolidin-1-yl]pyridazin-3-yl]phenol;
(42) 6-[6-(2,4-dichloro-6-hydroxy-phenyl)pyridazin-3-yl]-6-azaspiro[3.4]octan-2-ol;
(43) 3,5-dichloro-2-[6-[(2R)-2-[(dimethylamino)methyl]morpholin-4-yl]pyridazin-3-yl]phenol;
(44) 3-fluoro-2-[6-[(2S)-2-(hydroxymethyl)morpholin-4-yl]pyridazin-3-yl]phenol;
(45) 3-fluoro-2-[6-[(2S)-2-(hydroxymethyl)morpholin-4-yl]pyridazin-3-yl]-5-methyl-phenol;
(46) 2-[6-(2,4-dichloro-6-hydroxy-phenyl)pyridazin-3-yl]-2-azaspiro[3.3]heptan-6-ol;
(47) N-[7-[6-(2,4-dichloro-6-hydroxy-phenyl)pyridazin-3-yl]-7-azaspiro[3.5]nonan-2-yl]acetamide;
(48) 3-chloro-2-[6-[(2S)-2-(hydroxymethyl)morpholin-4-yl]pyridazin-3-yl]phenol;
(49) 3,5-dichloro-2-[6-(4,4-difluoro-1-piperidyl)pyridazin-3-yl]phenol;
(50) 3,5-dichloro-2-[6-[(3R)-3-[(dimethylamino)methyl]-1-piperidyl]pyridazin-3-yl]phenol;
(51) 3,5-dichloro-2-[6-[(3S)-3-[(dimethylamino)methyl]-1-piperidyl]pyridazin-3-yl]phenol;
(52) 2-[6-[2-(1-hydroxy-1-methyl-ethyl)morpholin-4-yl]pyridazin-3-yl]-3-methyl-5-(trifluoromethyl)phenol Enantiomer 1;
(53) 2-[6-[-2-(1-hydroxy-1-methyl-ethyl)morpholin-4-yl]pyridazin-3-yl]-3-methyl-5-(trifluoromethyl)phenol Enantiomer 2;
(54) 2-[6-[(2S)-2-(hydroxymethyl)morpholin-4-yl]pyridazin-3-yl]-3-(trifluoromethyl) phenol;
(55) 3,5-dichloro-2-[6-(2-methyl-2,7-diazaspiro[3.5]nonan-7-yl)pyridazin-3-yl]phenol;
(56) 2-[6-[(2S)-2-(hydroxymethyl)morpholin-4-yl]pyridazin-3-yl]-3-methyl-phenol;
(57) N-[[(3R)-1-[6-(2,4-dichloro-6-hydroxy-phenyl)pyridazin-3-yl]-3-piperidyl]methyl]acetamide;
(58) N-[[(3S)-1-[6-(2,4-dichloro-6-hydroxy-phenyl)pyridazin-3-yl]-3-piperidyl]methyl]acetamide;
(59) 2-[6-[-2-[(dimethylamino)methyl]morpholin-4-yl]pyridazin-3-yl]-3-methyl-5-(trifluoromethyl)phenol Enantiomer 1;
(60) 2-[6-[-2-[(dimethylamino)methyl]morpholin-4-yl]pyridazin-3-yl]-3-methyl-5-(trifluoromethyl)phenol Enantiomer 2;
(61) 1-[6-[2-hydroxy-6-methyl-4-(trifluoromethyl)phenyl]pyridazin-3-yl]pyrrolidin-2-one;
(62) (4S)-4-hydroxy-1-[6-[2-hydroxy-6-methyl-4-(trifluoromethyl)phenyl]pyridazin-3-yl]pyrrolidin-2-one;
(63) 5-chloro-2-[6-[(2S)-2-(hydroxymethyl)morpholin-4-yl]pyridazin-3-yl]-3-methyl-phenol;
(64) 3,5-dichloro-2-[6-(2-oxa-6-azaspiro[3.3]heptan-6-yl)pyridazin-3-yl]phenol;
(65) 2-[6-[(3R)-3-(hydroxymethyl)pyrrolidin-1-yl]pyridazin-3-yl]-3,5-dimethyl-phenol;
(66) 3,5-dichloro-2-[6-(2-oxa-7-azaspiro[3.5]nonan-7-yl)pyridazin-3-yl]phenol;
(67) (3S)-1-[6-(2-hydroxy-4,6-dimethyl-phenyl)pyridazin-3-yl]piperidin-3-ol;
(68) 2-[6-[(3S)-3-(ethylamino)-1-piperidyl]pyridazin-3-yl]-3,5-dimethyl-phenol;
(69) 2-[6-[(3S)-3-amino-1-piperidyl]pyridazin-3-yl]-3,5-dimethyl-phenol;
(70) (3R)-1-[6-(2-hydroxy-4,6-dimethyl-phenyl)pyridazin-3-yl]pyrrolidin-3-ol;
(71) (3S)-1-[6-(2-hydroxy-4,6-dimethyl-phenyl)pyridazin-3-yl]pyrrolidin-3-ol;
(72) 2-[4-ethyl-6-[(2S)-2-(hydroxymethyl)morpholin-4-yl]pyridazin-3-yl]-5-methyl-phenol;
(73) 5-chloro-2-[4-ethyl-6-[(2S)-2-(hydroxymethyl)morpholin-4-yl]pyridazin-3-yl]phenol;
(74) 2-[6-[(2S)-2-(hydroxymethyl)morpholin-4-yl]-4-isopropyl-pyridazin-3-yl]-5-methyl-phenol;
(75) 5-chloro-2-[6-[(2S)-2-(hydroxymethyl)morpholin-4-yl]-4-isopropyl-pyridazin-3-yl]phenol;
(76) 2-[6-[(2S)-2-(hydroxymethyl)morpholin-4-yl]-4-methyl-pyridazin-3-yl]-5-methyl-phenol;
(77) 2-[4-[(2S)-2-(hydroxymethyl)morpholin-4-yl]-6,7-dihydro-5H-cyclopenta[d]pyridazin-1-yl]-5-methyl-phenol;
(78) 5-chloro-2-[4-[(2S)-2-(hydroxymethyl)morpholin-4-yl]-6,7-dihydro-5H-cyclopenta[d]pyridazin-1-yl]phenol;
(79) 2-[6-[(3S)-3-(hydroxymethyl)pyrrolidin-1-yl]pyridazin-3-yl]-3,5-dimethyl-phenol;
(80) 2-[6-[(2S)-2-(hydroxymethyl)morpholin-4-yl]-4,5-dimethyl-pyridazin-3-yl]-5-methyl-phenol;
(81) 5-chloro-2-[6-[(2S)-2-(hydroxymethyl)morpholin-4-yl]-4,5-dimethyl-pyridazin-3-yl]phenol;
(82) 2-[6-[(3S)-3-(methoxymethyl)pyrrolidin-1-yl]pyridazin-3-yl]-3,5-dimethyl-phenol;
(83) 2-[6-(2-hydroxy-4,6-dimethyl-phenyl)pyridazin-3-yl]-2-azaspiro[3.5]nonan-7-ol;
(84) 2-[6-(2-hydroxy-4,6-dimethyl-phenyl)pyridazin-3-yl]-2-azaspiro[3.4]octan-6-ol;
(85) 5-chloro-2-[6-[(2S)-2-(hydroxymethyl)morpholin-4-yl]-4-methyl-pyridazin-3-yl]phenol;
(86) (4S)-4-hydroxy-1-[6-(2-hydroxy-4,6-dimethyl-phenyl)pyridazin-3-yl]pyrrolidin-2-one;
(87) 2-[6-[(2R)-2-(aminomethyl)morpholin-4-yl]pyridazin-3-yl]-3,5-dimethyl-phenol; 2,2,2-trifluoroacetic acid;
(88) 2-[6-[(3R)-3-(methoxymethyl)pyrrolidin-1-yl]pyridazin-3-yl]-3,5-dimethyl-phenol;
(89) 2-[6-[(2R)-2-(aminomethyl)morpholin-4-yl]pyridazin-3-yl]-3,5-dimethyl-phenol; 2,2,2-trifluoroacetic acid;
(90) 6-[(2S)-2-(hydroxymethyl)morpholin-4-yl]-3-(2-hydroxy-4-methyl-phenyl)pyridazine-4-carbonitrile;
(91) 3,5-dimethyl-2-[6-(1-methyl-1,6-diazaspiro[3.3]heptan-6-yl)pyridazin-3-yl]phenol;
(92) 2-[6-[(2S)-2-(hydroxymethyl)morpholin-4-yl]pyridazin-3-yl]-5-methyl-phenol;
(93) 5-fluoro-2-[6-[(2S)-2-(hydroxymethyl)morpholin-4-yl]-4-methyl-pyridazin-3-yl]phenol;
(94) 2-[6-(2-hydroxy-4,6-dimethyl-phenyl)pyridazin-3-yl]-2-azaspiro[3.4]octan-6-ol
Enantiomer 1;
(95) 2-[6-(2-hydroxy-4,6-dimethyl-phenyl)pyridazin-3-yl]-2-azaspiro[3.4]octan-6-ol
Enantiomer 2;
(96) 2-[6-[(2S)-2-(hydroxymethyl)morpholin-4-yl]pyridazin-3-yl]-5-methoxy-3-methyl-phenol;
(97) 2-[6-[(2S)-2-(hydroxymethyl)morpholin-4-yl]-4-methyl-pyridazin-3-yl]-5-(trifluoromethyl)phenol;
(98) 2-[6-[2-[(1S)-1-hydroxyethyl]morpholin-4-yl]pyridazin-3-yl]-3,5-dimethylphenol Diastereoisomer 2;
(99) 2-[6-[2-[(1S)-1-hydroxyethyl]morpholin-4-yl]pyridazin-3-yl]-3,5-dimethylphenol Diastereoisomer 1;
(102) 2-[6-[(2S)-2-(hydroxymethyl)morpholin-4-yl]pyridazin-3-yl]-4-methyl-phenol;
(103) 3-(4-chloro-2-hydroxy-phenyl)-6-[(2S)-2-(hydroxymethyl)morpholin-4-yl]pyridazine-4-carbonitrile;
(105) N-[[(2S)-4-[6-(2-hydroxy-4,6-dimethyl-phenyl)pyridazin-3-yl]morpholin-2-yl]methyl]acetamide;
(106) N-[[(2R)-4-[6-(2-hydroxy-4,6-dimethyl-phenyl)pyridazin-3-yl]morpholin-2-yl]methyl]acetamide;
(107) 2-[6-[3-(hydroxymethyl)azepan-1-yl]pyridazin-3-yl]-3,5-dimethyl-phenol;
(108) 2-[6-[2-[(1R)-1-hydroxyethyl]morpholin-4-yl]pyridazin-3-yl]-3,5-dimethyl-phenol Diastereoisomer 1;
(109) 2-[6-[(2S)-2-(hydroxymethyl)morpholin-4-yl]-5-methyl-pyridazin-3-yl]-3,5-dimethyl-phenol;
(110) 2-[6-[(2S)-2-(hydroxymethyl)morpholin-4-yl]-5-methyl-pyridazin-3-yl]-5-methyl-phenol;
(111) 2-[6-[rac-(4aS,8aS)-3,4a,5,7,8,8a-hexahydro-2H-pyrano[3,4-b][1,4]oxazin-1-yl]pyridazin-3-yl]-3,5-dimethyl-phenol;
(112) 2-[6-[(2S)-2-(hydroxymethyl)morpholin-4-yl]-4-methyl-pyridazin-3-yl]-3,5-dimethyl-phenol;
(113) (4R)-2-[6-(2-hydroxy-4,6-dimethyl-phenyl)pyridazin-3-yl]isoxazolidin-4-ol;
(114) 2-[6-[1-(hydroxymethyl)-6-azabicyclo[3.2.1]octan-6-yl]pyridazin-3-yl]-3,5-dimethyl-phenol;
(115) 2-[6-[(2S)-2-(hydroxymethyl)azetidin-1-yl]pyridazin-3-yl]-3,5-dimethyl-phenol;
(116) 2-[6-[(2S)-2-(hydroxymethyl)morpholin-4-yl]-4-methyl-pyridazin-3-yl]-5-methoxy-phenol;
(117) 3,5-dichloro-2-[6-[(2S)-2-(hydroxymethyl)azetidin-1-yl]pyridazin-3-yl]phenol;
(118) 2-[6-[(2R)-2-(hydroxymethyl)azetidin-1-yl]pyridazin-3-yl]-3,5-dimethyl-phenol;
(119) 2-[6-[(2S)-2-(hydroxymethyl)morpholin-4-yl]-4-methyl-pyridazin-3-yl]-5-methoxy-3-methyl-phenol;
(120) 3,5-dimethyl-2-[6-(3-oxa-8-azabicyclo[3.2.1]octan-8-yl)pyridazin-3-yl]phenol;
(121) 2-[6-[(2S)-2-(hydroxymethyl)morpholin-4-yl]pyridazin-3-yl]-3-methyl-5-(trifluoromethoxy)phenol;
(122) 1-[6-(2-hydroxy-4,6-dimethyl-phenyl)pyridazin-3-yl]-3-(hydroxymethyl) pyrrolidin-2-one;
(123) 2-[6-[(3R)-3-(aminomethyl)morpholin-4-yl]pyridazin-3-yl]-3,5-dimethyl-phenol, 2,2,2-trifluoroacetic acid;
(124) 2-[6-[(3S)-3-(aminomethyl)morpholin-4-yl]pyridazin-3-yl]-3,5-dimethyl-phenol;
(125) 2-[6-[(4aR,7aR)-3,4,4a,5,7,7a-hexahydro-2H-pyrrolo[3,4-b][1,4]oxazin-6-yl]pyridazin-3-yl]-3,5-dimethylphenol;
(126) 1-[4-[6-(2-hydroxy-4,6-dimethylphenyl)pyridazin-3-yl]morpholin-2-yl]propan-1-one;
(127) 5-chloro-2-[6-[(3S)-3-(hydroxymethyl)pyrrolidin-1-yl]pyridazin-3-yl]-3-methyl-phenol;
(128) 2-[6-[(3S)-3-(dimethylamino)-1-piperidyl]pyridazin-3-yl]-3,5-dimethyl-phenol;
(129) N-[[(3R)-4-[6-(2-hydroxy-4,6-dimethyl-phenyl)pyridazin-3-yl]morpholin-3-yl]methyl]acetamide;
(130) (4S)-1-[6-(4-chloro-2-hydroxy-6-methyl-phenyl)pyridazin-3-yl]-4-hydroxy-pyrrolidin-2-one;
(131) (4S)-4-amino-1-[6-(2-hydroxy-4,6-dimethyl-phenyl)pyridazin-3-yl]pyrrolidin-2-one;
(132) (4R)-4-hydroxy-1-[6-[2-hydroxy-6-methyl-4-(trifluoromethyl)phenyl]pyridazin-3-yl]pyrrolidin-2-one;
(133) 5-chloro-2-[6-[(2S)-2-(hydroxymethyl)morpholin-4-yl]-5-methyl-pyridazin-3-yl]-3-methyl-phenol;
(134) 5-(difluoromethyl)-2-[6-[(2S)-2-(hydroxymethyl)morpholin-4-yl]pyridazin-3-yl]-3-methyl-phenol;
(135) 2-[6-[(2S)-2-(1-aminocyclopropyl)morpholin-4-yl]pyridazin-3-yl]-3,5-dimethyl-phenol;
(136) 2-[6-[(2R)-2-(1-aminocyclopropyl)morpholin-4-yl]pyridazin-3-yl]-3,5-dimethyl-phenol;
(137) (4R)-1-[6-(2-hydroxy-4,6-dimethyl-phenyl)pyridazin-3-yl]-4-(hydroxymethyl) pyrrolidin-2-one;
(138) (4S)-1-[6-(2-hydroxy-4,6-dimethyl-phenyl)pyridazin-3-yl]-4-(hydroxymethyl) pyrrolidin-2-one;
(139) (3R)-1-[6-(2-hydroxy-4,6-dimethyl-phenyl)pyridazin-3-yl]-3-(hydroxymethyl) pyrrolidin-2-one;
(140) (3S)-1-[6-(2-hydroxy-4,6-dimethyl-phenyl)pyridazin-3-yl]-3-(hydroxymethyl) pyrrolidin-2-one;
(141) 2-[6-[(2S)-2-(hydroxymethyl)morpholin-4-yl]pyridazin-3-yl]-3,6-dimethylphenol;
(142) 2-[(2R)-4-[6-(2-hydroxy-4,6-dimethylphenyl)pyridazin-3-yl]morpholin-2-yl]-N-methylacetamide;
(143) 2-[(2S)-4-[6-(2-hydroxy-4,6-dimethylphenyl)pyridazin-3-yl]morpholin-2-yl]-N-methylacetamide;
(144) 2-[4-(difluoromethyl)-6-[(2S)-2-(hydroxymethyl)morpholin-4-yl]pyridazin-3-yl]-5-methyl-phenol;
(145) 5-chloro-2-[4-(difluoromethyl)-6-[(2S)-2-(hydroxymethyl)morpholin-4-yl]pyridazin-3-yl]phenol;
(146) 2-[(2R)-4-[6-(2-hydroxy-4,6-dimethylphenyl)pyridazin-3-yl]morpholin-2-yl]acetamide;
(147) 2-[(2S)-4-[6-(2-hydroxy-4,6-dimethylphenyl)pyridazin-3-yl]morpholin-2-yl]acetamide;
(148) 2-[6-[(3R)-3-(dimethylamino)-1-piperidyl]pyridazin-3-yl]-3,5-dimethyl-phenol;
(149) 5-chloro-2-[6-[(2S)-2-(hydroxymethyl)morpholin-4-yl]-4-methyl-pyridazin-3-yl]-3-methyl-phenol;
(150) 2-[(2R)-4-[6-(2-hydroxy-4,6-dimethylphenyl)pyridazin-3-yl]morpholin-2-yl]-N,N-dimethylacetamide;
(151) 2-[(2S)-4-[6-(2-hydroxy-4,6-dimethylphenyl)pyridazin-3-yl]morpholin-2-yl]-N,N-dimethylacetamide;
(152) 2-[6-[(2S)-2-(hydroxymethyl)morpholin-4-yl]pyridazin-3-yl]-4,6-dimethylphenol;
(153) 3-(difluoromethyl)-2-[6-[(2S)-2-(hydroxymethyl)morpholin-4-yl]pyridazin-3-yl]-5-methyl-phenol;
(154) 3,5-dimethyl-2-[6-[rac-(4aR,8aS)-3,4a,5,7,8,8a-hexahydro-2H-pyrano[3,4-b][1,4]oxazin-1-yl]pyridazin-3-yl]phenol;
(155) 2-[6-[(2S)-2-(hydroxymethyl)morpholin-4-yl]-4-(trifluoromethyl)pyridazin-3-yl]-5-methyl-phenol;
(156) 1-[(2R)-4-[6-(2-hydroxy-4,6-dimethylphenyl)pyridazin-3-yl]morpholin-2-yl]propan-1-one;
(157) 1-[(2S)-4-[6-(2-hydroxy-4,6-dimethylphenyl)pyridazin-3-yl]morpholin-2-yl]propan-1-one;
(158) 6-(2-hydroxy-4,6-dimethyl-phenyl)-3-[(2S)-2-(hydroxymethyl)morpholin-4-yl]pyridazine-4-carbonitrile;
(159) 3-[(2S)-2-(hydroxymethyl)morpholin-4-yl]-6-[2-hydroxy-6-methyl-4-(trifluoromethyl)phenyl]pyridazine-4-carbonitrile;
(160) 5-chloro-2-[6-[(2S)-2-(hydroxymethyl)morpholin-4-yl]-4-(trifluoromethyl) pyridazin-3-yl]phenol;
(161) 3,5-dichloro-2-[6-[2-(fluoromethyl)morpholin-4-yl]pyridazin-3-yl]phenol;
(162) 4-[6-(2-hydroxy-4,6-dimethyl-phenyl)pyridazin-3-yl]-2,3-dihydro-1,4-benzoxazin-6-ol;
(163) 6-(4-chloro-2-hydroxy-6-methyl-phenyl)-3-[(2S)-2-(hydroxymethyl)morpholin-4-yl]pyridazine-4-carbonitrile;
(164) N-[[(3S)-1-[6-[2-hydroxy-6-methyl-4-(trifluoromethyl)phenyl]pyridazin-3-yl]-3-piperidyl]methyl]acetamide;
(165) 2-[6-[(2S)-2-[dideuterio(hydroxy)methyl]morpholin-4-yl]pyridazin-3-yl]-3,5-dimethylphenol;
(166) (2S)-4-[6-(2-hydroxy-4,6-dimethylphenyl)pyridazin-3-yl]-N-methylmorpholine-2-carboxamide;
(167) N-[[(3R)-1-[6-[2-hydroxy-6-methyl-4-(trifluoromethyl)phenyl]pyridazin-3-yl]-3-piperidyl]methyl]acetamide;
(168) 2-[6-[(4aR,7aR)-4-ethyl-2,3,4a,5,7,7a-hexahydropyrrolo[3,4-b][1,4]oxazin-6-yl]pyridazin-3-yl]-3,5-dimethylphenol;
(169) 5-chloro-2-[6-[(2S)-2-[dideuterio(hydroxy)methyl]morpholin-4-yl]pyridazin-3-yl]-3-methylphenol;
(170) 2-[5-(difluoromethyl)-6-[(2S)-2-(hydroxymethyl)morpholin-4-yl]pyridazin-3-yl]-3,5-dimethyl-phenol;
(171) 5-chloro-2-[5-(difluoromethyl)-6-[(2S)-2-(hydroxymethyl)morpholin-4-yl]pyridazin-3-yl]-3-methyl-phenol;
(172) 3-chloro-2-[6-[(2S)-2-(hydroxymethyl)morpholin-4-yl]pyridazin-3-yl]-5-methyl-phenol;
(173) 2-[6-[(2R,6S)-2,6-bis(hydroxymethyl)morpholin-4-yl]pyridazin-3-yl]-3,5-dimethyl-phenol;
(174) 2-[6-[2-(fluoromethyl)morpholin-4-yl]pyridazin-3-yl]-3,5-dimethyl-phenol;
(175) 6-[6-(2-hydroxy-4,6-dimethyl-phenyl)pyridazin-3-yl]-6-azaspiro[3.5]nonan-2-ol
Isomer 1;
(176) 6-[6-(2-hydroxy-4,6-dimethyl-phenyl)pyridazin-3-yl]-6-azaspiro[3.5]nonan-2-ol
Isomer 2;
(177) 3-chloro-2-[6-[(2S)-2-(hydroxymethyl)morpholin-4-yl]-4-methyl-pyridazin-3-yl]-5-methyl-phenol;
(178) 2-[6-[(2R,6S)-2,6-bis(hydroxymethyl)morpholin-4-yl]pyridazin-3-yl]-3-methyl-5-(trifluoromethyl)phenol;
(179) (4S)-2-[6-(2-hydroxy-4,6-dimethyl-phenyl)pyridazin-3-yl]isoxazolidin-4-ol;
(180) 2-[6-[2-[(1R)-1-hydroxyethyl]morpholin-4-yl]pyridazin-3-yl]-3,5-dimethylphenol Diastereoisomer 2;
(181) (2S)-4-[6-(2-hydroxy-4,6-dimethylphenyl)pyridazin-3-yl]morpholine-2-carboxamide;
(182) (2R)-4-[6-(2-hydroxy-4,6-dimethylphenyl)pyridazin-3-yl]-N-methylmorpholine-2-carboxamide;
(183) (2R)-4-[6-(2-hydroxy-4,6-dimethylphenyl)pyridazin-3-yl]morpholine-2-carboxamide;
(184) (2R)-4-[6-(2-hydroxy-4,6-dimethylphenyl)pyridazin-3-yl]-N,N-dimethylmorpholine-2-carboxamide;
(185) 2-[6-[(4aS,7aS)-3,4,4a,5,7,7a-hexahydro-2H-pyrrolo[3,4-b][1,4]oxazin-6-yl]pyridazin-3-yl]-3,5-dimethylphenol;
(186) 2-[4-(hydroxymethyl)-6-[(2S)-2-(hydroxymethyl)morpholin-4-yl]pyridazin-3-yl]-3,5-dimethyl-phenol;
(187) 2-[5-(hydroxymethyl)-6-[(2S)-2-(hydroxymethyl)morpholin-4-yl]pyridazin-3-yl]-3,5-dimethyl-phenol;
(188) 5-chloro-2-[4-(hydroxymethyl)-6-[(2S)-2-(hydroxymethyl)morpholin-4-yl]pyridazin-3-yl]-3-methyl-phenol;
(189) 5-chloro-2-[5-(hydroxymethyl)-6-[(2S)-2-(hydroxymethyl)morpholin-4-yl]pyridazin-3-yl]-3-methyl-phenol;
(190) 2-[6-[rac-(4aR,7aR)-3,4a,5,6,7,7a-hexahydro-2H-pyrrolo[3,4-b][1,4]oxazin-4-yl]pyridazin-3-yl]-3,5-dimethylphenol;
(191) 2-[6-[(2S)-2-(hydroxymethyl)morpholin-4-yl]pyridazin-3-yl]-3,4-dimethylphenol;
(192) 2-[6-[(2S)-2-[dideuterio(hydroxy)methyl]morpholin-4-yl]pyridazin-3-yl]-3-methyl-5-(trifluoromethyl)phenol;
(193) 4-[6-(4-chloro-2-hydroxy-6-methyl-phenyl)pyridazin-3-yl]-2,3-dihydro-1,4-benzoxazin-7-ol;
(194) 2-[5-(difluoromethyl)-6-[(2S)-2-(hydroxymethyl)morpholin-4-yl]pyridazin-3-yl]-3-methyl-5-(trifluoromethyl)phenol;
(195) 2-[6-[rac-(4aR,8aS)-2,3,4a,5,6,7,8,8a-octahydropyrido[4,3-b][1,4]oxazin-4-yl]pyridazin-3-yl]-3,5-dimethyl-phenol;
(196) 3,5-dimethyl-2-[6-(2-oxa-6,9-diazaspiro[4.5]decan-9-yl)pyridazin-3-yl]phenol;
(197) 2-[6-[rac-(4aR,8aS)-2,3,4a,5,6,7,8,8a-octahydropyrido[4,3-b][1,4]oxazin-4-yl]pyridazin-3-yl]-3-methyl-5-(trifluoromethyl)phenol;
(198) 2-[6-[(2S,6S)-2,6-bis(hydroxymethyl)morpholin-4-yl]pyridazin-3-yl]-3,5-dimethyl-phenol;
(199) 2-[6-[(2S,6S)-2,6-bis(hydroxymethyl)morpholin-4-yl]pyridazin-3-yl]-3-methyl-5-(trifluoromethyl)phenol;
(200) 2-[6-[(2R,6R)-2,6-bis(hydroxymethyl)morpholin-4-yl]pyridazin-3-yl]-3-methyl-5-(trifluoromethyl)phenol;
(201) 2-[6-[(2R,6R)-2,6-bis(hydroxymethyl)morpholin-4-yl]pyridazin-3-yl]-3,5-dimethyl-phenol;
(203) 3,5-dichloro-2-(6-thiomorpholinopyridazin-3-yl)phenol;
(204) 5-cyclopropyl-2-[6-[(2S)-2-(hydroxymethyl)morpholin-4-yl]pyridazin-3-yl]-3-methyl-phenol;
(206) 2-[6-[(3aS,6aR)-1,3,3a,4,6,6a-hexahydrofuro[3,4-c]pyrrol-5-yl]pyridazin-3-yl]-3,5-dimethyl-phenol;
(207) 2-[6-[(3aR,6aR)-1,3,3a,4,6,6a-hexahydrofuro[3,4-c]pyrrol-5-yl]pyridazin-3-yl]-3,5-dimethyl-phenol;
(208) 2-[6-(8-ethyl-1,11-dioxa-4,8-diazaspiro[5.6]dodecan-4-yl)pyridazin-3-yl]-3,5-dimethylphenol;
(209) 2-[6-[rac-(4aR,8aS)-6-ethyl-3,4a,5,7,8,8a-hexahydro-2H-pyrido[4,3-b][1,4]oxazin-4-yl]pyridazin-3-yl]-3,5-dimethyl-phenol;
(210) 2-[6-[rac-(4aR,8aS)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol;
(211) N-[[(3S)-1-[6-(4-chloro-2-hydroxy-6-methyl-phenyl)pyridazin-3-yl]-3-piperidyl]methyl]acetamide;
(212) N-[[(3R)-1-[6-(4-chloro-2-hydroxy-6-methyl-phenyl)pyridazin-3-yl]-3-piperidyl]methyl]acetamide;
(213) 1-[rac-(4aR,8aS)-4-[6-(2-hydroxy-4,6-dimethyl-phenyl)pyridazin-3-yl]-3,4a,5,7,8,8a-hexahydro-2H-pyrido[4,3-b][1,4]oxazin-6-yl]ethanone;
(214) 1-[rac-(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(215) 2-[6-(2,3-dihydropyrido[3,4-b][1,4]oxazin-1-yl)pyridazin-3-yl]-3,5-dimethyl-phenol;
(216) N-[[(2R)-4-[6-[2-hydroxy-6-methyl-4-(trifluoromethyl)phenyl]pyridazin-3-yl]morpholin-2-yl]methyl]acetamide;
(217) 1-[6-(2-hydroxy-4,6-dimethyl-phenyl)pyridazin-3-yl]-3,4-dihydro-2H-quinolin-7-ol;
(219) 2-[6-[(2S)-2-(fluoromethyl)morpholin-4-yl]pyridazin-3-yl]-3,5-dimethyl-phenol;
(220) 2-[6-[(2R)-2-(fluoromethyl)morpholin-4-yl]pyridazin-3-yl]-3,5-dimethyl-phenol;
(221) 2-[6-[rac-(4aR,8aS)-2,3,4a,5,6,7,8,8a-octahydropyrido[4,3-b][1,4]oxazin-4-yl]pyridazin-3-yl]-5-chloro-3-methyl-phenol;
(222) 1-[rac-(4aR,8aS)-4-[6-(4-chloro-2-hydroxy-6-methyl-phenyl)pyridazin-3-yl]-3,4a,5,7,8,8a-hexahydro-2H-pyrido[4,3-b][1,4]oxazin-6-yl]ethanone;
(223) 2-[6-[rac-(4aR,8aS)-6-ethyl-3,4a,5,7,8,8a-hexahydro-2H-pyrido[4,3-b][1,4]oxazin-4-yl]pyridazin-3-yl]-5-chloro-3-methyl-phenol;
(224) 2-[6-[rac-(4aR,8aS)-6-methyl-3,4a,5,7,8,8a-hexahydro-2H-pyrido[4,3-b][1,4]oxazin-4-yl]pyridazin-3-yl]-5-chloro-3-methyl-phenol;
(225) 2-[6-[rac-(4aR,8aS)-6-methyl-3,4a,5,7,8,8a-hexahydro-2H-pyrido[4,3-b][1,4]oxazin-4-yl]pyridazin-3-yl]-3,5-dimethyl-phenol;
(226) 2-[6-[rac-(4aR,8aS)-6-methyl-3,4a,5,7,8,8a-hexahydro-2H-pyrido[4,3-b][1,4]oxazin-4-yl]pyridazin-3-yl]-3-methyl-5-(trifluoromethyl)phenol;
(227) 2-[6-(6-ethyl-2-oxa-6,9-diazaspiro[4.5]decan-9-yl)pyridazin-3-yl]-3,5-dimethylphenol;
(228) 1-[rac-(3aS,6aS)-1-[6-[2-hydroxy-6-methyl-4-(trifluoromethyl)phenyl]pyridazin-3-yl]-2,3,3a,4,6,6a-hexahydropyrrolo[3,4-b]pyrrol-5-yl]ethanone;
(229) 2-[6-[rac-(3aS,6aS)-3,3a,4,5,6,6a-hexahydro-2H-pyrrolo[3,4-b]pyrrol-1-yl]pyridazin-3-yl]-3-methyl-5-(trifluoromethyl)phenol;
(230) 1-[6-(2-hydroxy-4,6-dimethyl-phenyl)pyridazin-3-yl]-3,4-dihydro-2H-quinolin-6-ol;
(231) 1-[6-(2-hydroxy-4,6-dimethyl-phenyl)pyridazin-3-yl]-3,4-dihydro-2H-quinolin-8-ol;
(232) 1-[6-(2-hydroxy-4,6-dimethyl-phenyl)pyridazin-3-yl]-3,4-dihydro-2H-quinolin-5-ol;
(233) 2-[6-[(2R)-2-(2-hydroxyethyl)morpholin-4-yl]pyridazin-3-yl]-3,5-dimethyl-phenol;
(234) 2-[6-[(2S)-2-(2-hydroxyethyl)morpholin-4-yl]pyridazin-3-yl]-3,5-dimethyl-phenol;
(235) 1-[6-(2-hydroxy-4,6-dimethyl-phenyl)pyridazin-3-yl]-5-(hydroxymethyl) piperidin-2-one;
(236) 1-[6-(2-hydroxy-4,6-dimethyl-phenyl)pyridazin-3-yl]indolin-5-ol;
(237) 3,5-dichloro-2-[6-[(2R)-2-(fluoromethyl)morpholin-4-yl]pyridazin-3-yl]phenol;
(238) 3,5-dichloro-2-[6-[(2S)-2-(fluoromethyl)morpholin-4-yl]pyridazin-3-yl]phenol;
(239) 1-[6-(2,4-dichloro-6-hydroxy-phenyl)pyridazin-3-yl]hexahydropyrimidin-2-one;
(240) 1-[6-(2-hydroxy-4,6-dimethyl-phenyl)pyridazin-3-yl]indolin-4-ol;
(241) 2-[6-[4-(hydroxymethyl)indolin-1-yl]pyridazin-3-yl]-3,5-dimethyl-phenol;
(242) 1-[6-(2-hydroxy-4,6-dimethyl-phenyl)pyridazin-3-yl]-3,4-dihydro-2H-1,6-naphthyridin-5-ol;
(243) 2-[6-[rac-(3aS,6aS)-5-ethyl-2,3,3a,4,6,6a-hexahydropyrrolo[3,4-b]pyrrol-1-yl]pyridazin-3-yl]-3-methyl-5-(trifluoromethyl)phenol;
(245) 5-ethyl-2-[6-[(2S)-2-(hydroxymethyl)morpholin-4-yl]pyridazin-3-yl]-3-methylphenol;
(246) 3-ethyl-2-[6-[(2S)-2-(hydroxymethyl)morpholin-4-yl]pyridazin-3-yl]-5-methylphenol;
(247) 2-[4-(difluoromethyl)-6-[(2S)-2-(hydroxymethyl)morpholin-4-yl]pyridazin-3-yl]-5-(trifluoromethyl)phenol;
(248) 2-[4-(difluoromethyl)-6-[(2S)-2-(hydroxymethyl)morpholin-4-yl]pyridazin-3-yl]-3-methyl-5-(trifluoromethyl)phenol;
(249) 5-chloro-2-[4-(difluoromethyl)-6-[(2S)-2-(hydroxymethyl)morpholin-4-yl]pyridazin-3-yl]-3-methyl-phenol;
(250) (4S)-4-amino-1-[6-[2-hydroxy-6-methyl-4-(trifluoromethyl)phenyl]pyridazin-3-yl]pyrrolidin-2-one;
(251) 2-[6-[(4aR,8aS)-2,3,4a,5,6,7,8,8a-octahydropyrido[4,3-b][1,4]oxazin-4-yl]pyridazin-3-yl]-3-methyl-5-(trifluoromethyl)phenol;
(252) 2-[6-[(4aS,8aR)-2,3,4a,5,6,7,8,8a-octahydropyrido[4,3-b][1,4]oxazin-4-yl]pyridazin-3-yl]-3-methyl-5-(trifluoromethyl)phenol;
(253) 1-[6-(2-hydroxy-4,6-dimethyl-phenyl)pyridazin-3-yl]indolin-7-ol;
(254) 1-[6-(2-hydroxy-4,6-dimethyl-phenyl)pyridazin-3-yl]indolin-6-ol;
(255) 2-[6-[(4aR,8aS)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol;
(256) 2-[6-[(4aS,8aR)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol;
(257) 1-[rac-(4aS,7aS)-4-[6-(2-hydroxy-4,6-dimethylphenyl)pyridazin-3-yl]-2,3,4a,5,7,7a-hexahydropyrrolo[3,4-b][1,4]oxazin-6-yl]ethanone;
(258) 3-methyl-2-[6-[(2R)-2-methylmorpholin-4-yl]pyridazin-3-yl]-5-(trifluoromethyl)phenol;
(259) 3-methyl-2-[6-[(2S)-2-methylmorpholin-4-yl]pyridazin-3-yl]-5-(trifluoromethyl)phenol;
(260) 2-[6-[(4aR,8aS)-6-methyl-3,4a,5,7,8,8a-hexahydro-2H-pyrido[4,3-b][1,4]oxazin-4-yl]pyridazin-3-yl]-3-methyl-5-(trifluoromethyl)phenol;
(261) 2-[6-[(4aS,8aR)-6-methyl-3,4a,5,7,8,8a-hexahydro-2H-pyrido[4,3-b][1,4]oxazin-4-yl]pyridazin-3-yl]-3-methyl-5-(trifluoromethyl)phenol;
(262) 1-[(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(263) 1-[(4aS,8aR)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(264) rac-(3aS,7aR)-5-[6-[2-hydroxy-6-methyl-4-(trifluoromethyl)phenyl]pyridazin-3-yl]-2,3,3a,6,7,7a-hexahydro-1H-pyrrolo[3,4-c]pyridin-4-one,
hydrochloride;
(265) 2-[6-[rac-(3aS,6aS)-5-methyl-2,3,3a,4,6,6a-hexahydropyrrolo[3,4-b]pyrrol-1-yl]pyridazin-3-yl]-3-methyl-5-(trifluoromethyl)phenol;
(266) 2-[6-[(2R)-2-(hydroxymethyl)morpholin-4-yl]pyridazin-3-yl]-3-methyl-5-(trifluoromethyl)phenol;
(267) N-[[(2R)-4-[6-(4-chloro-2-hydroxy-6-methyl-phenyl)pyridazin-3-yl]morpholin-2-yl]methyl]acetamide;
(268) 2-[6-[(4aR,8aS)-6-methyl-3,4a,5,7,8,8a-hexahydro-2H-pyrido[4,3-b][1,4]oxazin-4-yl]-4-(difluoromethyl)pyridazin-3-yl]-5-(trifluoromethyl)phenol;
(269) rac-(3aS,7aS)-5-[6-[2-hydroxy-6-methyl-4-(trifluoromethyl)phenyl]pyridazin-3-yl]-2,3,3a,6,7,7a-hexahydro-1H-pyrrolo[3,4-c]pyridin-4-one, hydrochloride;
(270) 2-[6-[(3aR,6aR)-5-methyl-2,3,3a,4,6,6a-hexahydropyrrolo[3,4-b]pyrrol-1-yl]pyridazin-3-yl]-3-methyl-5-(trifluoromethyl)phenol;
(271) 2-[6-[(3aS,6aS)-5-methyl-2,3,3a,4,6,6a-hexahydropyrrolo[3,4-b]pyrrol-1-yl]pyridazin-3-yl]-3-methyl-5-(trifluoromethyl)phenol;
(272) 2-[6-(3,4-dihydro-2H-quinolin-1-yl)pyridazin-3-yl]-3,5-dimethyl-phenol;
(273) 3-(hydroxymethyl)-1-[6-[2-hydroxy-6-methyl-4-(trifluoromethyl)phenyl]pyridazin-3-yl]piperidin-2-one;
(274) 2-[6-[(2S)-2-(hydroxymethyl)morpholin-4-yl]pyridazin-3-yl]-5-isopropyl-3-methyl-phenol;
(275) N-[[(2S)-4-[6-[2-hydroxy-6-methyl-4-(trifluoromethyl)phenyl]pyridazin-3-yl]morpholin-2-yl]methyl]acetamide;
(276) N-[[(2S)-4-[6-[2-hydroxy-6-methyl-4-(trifluoromethyl)phenyl]pyridazin-3-yl]morpholin-2-yl]methyl]-2-methylpropanamide;
(277) N-[2-[(2R)-4-[6-[2-hydroxy-6-methyl-4-(trifluoromethyl)phenyl]pyridazin-3-yl]morpholin-2-yl]ethyl]acetamide;
(278) N-[2-[(2S)-4-[6-[2-hydroxy-6-methyl-4-(trifluoromethyl)phenyl]pyridazin-3-yl]morpholin-2-yl]ethyl]acetamide;
(279) N-[[(2R)-4-[6-(4-chloro-2-hydroxy-6-methylphenyl)pyridazin-3-yl]morpholin-2-yl]methyl]-2-methylpropanamide;
(280) N-[[(2S)-4-[6-(4-chloro-2-hydroxy-6-methylphenyl)pyridazin-3-yl]morpholin-2-yl]methyl]acetamide;
(283) 2-[6-[(2R)-2-(fluoromethyl)morpholin-4-yl]pyridazin-3-yl]-3-methyl-5-(trifluoromethyl)phenol;
(284) 2-[6-[(2S)-2-(fluoromethyl)morpholin-4-yl]pyridazin-3-yl]-3-methyl-5-(trifluoromethyl)phenol;
(285) N-[[(2S)-4-[6-(4-chloro-2-hydroxy-6-methylphenyl)pyridazin-3-yl]morpholin-2-yl]methyl]-2-methylpropanamide;
(286) (4S)-4-hydroxy-1-[6-[2-hydroxy-6-methyl-4-(trifluoromethyl)phenyl]pyridazin-3-yl]piperidin-2-one;
(287) (3R)-3-(hydroxymethyl)-1-[6-[2-hydroxy-6-methyl-4-(trifluoromethyl)phenyl]pyridazin-3-yl]piperidin-2-one;
(288) (3S)-3-(hydroxymethyl)-1-[6-[2-hydroxy-6-methyl-4-(trifluoromethyl)phenyl]pyridazin-3-yl]piperidin-2-one;
(289) 3-methyl-2-[6-[rac-(4aR,7aR)-6-methyl-2,3,4a,5,7,7a-hexahydropyrrolo[3,4-b][1,4]oxazin-4-yl]pyridazin-3-yl]-5-(trifluoromethyl)phenol;
(290) (4aR,8aR)-1-[6-(2-hydroxy-4,6-dimethyl-phenyl)pyridazin-3-yl]-7-methyl-3,4,4a,5,6,8a-hexahydro-2H-1,7-naphthyridin-8-one;
(291) 2-[6-[(4aS,8aR)-6-methyl-3,4a,5,7,8,8a-hexahydro-2H-pyrido[4,3-b][1,4]oxazin-4-yl]-5-(difluoromethyl)pyridazin-3-yl]-3,5-dimethyl-phenol;
(292) (4R)-4-(hydroxymethyl)-1-[6-[2-hydroxy-6-methyl-4-(trifluoromethyl)phenyl]pyridazin-3-yl]piperidin-2-one;
(293) (4S)-4-(hydroxymethyl)-1-[6-[2-hydroxy-6-methyl-4-(trifluoromethyl)phenyl]pyridazin-3-yl]piperidin-2-one;
(295) 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-5-methyl-phenol;
(296) 2-[6-[(2S,6R)-2-(hydroxymethyl)-6-methyl-morpholin-4-yl]pyridazin-3-yl]-3-methyl-5-(trifluoromethyl)phenol;
(297) 2-[6-[(2S,6S)-2-(hydroxymethyl)-6-methyl-morpholin-4-yl]pyridazin-3-yl]-3-methyl-5-(trifluoromethyl)phenol;
(298) 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-5-(trifluoromethyl)phenol;
(299) 2-[6-[(4aR,8aS)-6-methyl-3,4a,5,7,8,8a-hexahydro-2H-pyrido[4,3-b][1,4]oxazin-4-yl]pyridazin-3-yl]-3-methyl-phenol;
(300) 1-[6-(2-hydroxy-4,6-dimethyl-phenyl)pyridazin-3-yl]-2,3,4,4a,5,6,7,8a-octahydro-1,7-naphthyridin-8-one;
(301) 2-[6-[(4aR,8aR)-6-methyl-3,4a,5,7,8,8a-hexahydro-2H-pyrido[4,3-b][1,4]oxazin-4-yl]pyridazin-3-yl]-3-methyl-5-(trifluoromethyl)phenol;
(302) 2-[6-[(2R,6R)-2-(hydroxymethyl)-6-methyl-morpholin-4-yl]pyridazin-3-yl]-3-methyl-5-(trifluoromethyl)phenol;
(303) 3,5-dimethyl-2-[6-[(2S)-2-(sulfanylmethyl)morpholin-4-yl]pyridazin-3-yl]phenol;
(304) 1-[rac-(4aR,8aS)-4-[6-(4-chloro-2-hydroxy-6-methyl-phenyl)pyridazin-3-yl]-3,4a,5,7,8,8a-hexahydro-2H-pyrido[4,3-b][1,4]oxazin-6-yl]ethanone;
(305) 3,5-dimethyl-2-[6-[(2R)-2-(sulfanylmethyl)morpholin-4-yl]pyridazin-3-yl]phenol;
(306) 2-[6-[(4aR,8aS)-6-methyl-3,4a,5,7,8,8a-hexahydro-2H-pyrido[4,3-b][1,4]oxazin-4-yl]-4-(difluoromethyl)pyridazin-3-yl]-5-methyl-phenol;
(307) 5-chloro-3-methyl-2-[6-[rac-(4aR,8aS)-6-methyl-3,4a,5,7,8,8a-hexahydro-2H-pyrido[4,3-b][1,4]oxazin-4-yl]pyridazin-3-yl]phenol;
(308) 2-[6-[(4aR,8aS)-2,3,4a,5,6,7,8,8a-octahydropyrido[4,3-b][1,4]oxazin-4-yl]pyridazin-3-yl]-5-chloro-3-methyl-phenol;
(309) 2-[6-[(4aS,8aR)-2,3,4a,5,6,7,8,8a-octahydropyrido[4,3-b][1,4]oxazin-4-yl]pyridazin-3-yl]-5-chloro-3-methyl-phenol;
(310) 2-[6-[(4aR,8aS)-6-methyl-3,4a,5,7,8,8a-hexahydro-2H-pyrido[4,3-b][1,4]oxazin-4-yl]pyridazin-3-yl]-3,5-dimethyl-phenol;
(311) 2-[6-[(4aS,8aR)-6-methyl-3,4a,5,7,8,8a-hexahydro-2H-pyrido[4,3-b][1,4]oxazin-4-yl]pyridazin-3-yl]-3,5-dimethyl-phenol;
(312) 2-[6-[(4aR,8aS)-6-ethyl-3,4a,5,7,8,8a-hexahydro-2H-pyrido[4,3-b][1,4]oxazin-4-yl]pyridazin-3-yl]-5-chloro-3-methyl-phenol;
(313) 2-[6-[(4aS,8aR)-6-ethyl-3,4a,5,7,8,8a-hexahydro-2H-pyrido[4,3-b][1,4]oxazin-4-yl]pyridazin-3-yl]-5-chloro-3-methyl-phenol;
(314) 2-[6-[(4aR,8aS)-2,3,4a,5,6,7,8,8a-octahydropyrido[4,3-b][1,4]oxazin-4-yl]pyridazin-3-yl]-3,5-dimethyl-phenol;
(315) 2-[6-[(4aS,8aR)-2,3,4a,5,6,7,8,8a-octahydropyrido[4,3-b][1,4]oxazin-4-yl]pyridazin-3-yl]-3,5-dimethyl-phenol;
(316) (4R)-4-hydroxy-1-[6-[2-hydroxy-6-methyl-4-(trifluoromethyl)phenyl]pyridazin-3-yl]piperidin-2-one;
(317) 2-[6-(4-ethyl-1,11-dioxa-4,8-diazaspiro[5.6]dodecan-8-yl)pyridazin-3-yl]-3,5-dimethylphenol;
(318) 3-methyl-2-[6-[rac-(4aR,7aR)-3,4a,5,6,7,7a-hexahydro-2H-pyrrolo[3,4-b][1,4]oxazin-4-yl]pyridazin-3-yl]-5-(trifluoromethyl)phenol; hydrochloride;
(320) 3-methyl-2-[6-[rac-(4aR,7aS)-3,4a,5,6,7,7a-hexahydro-2H-pyrrolo[3,4-b][1,4]oxazin-4-yl]pyridazin-3-yl]-5-(trifluoromethyl)phenol; hydrochloride;
(321) 2-[6-[(4aS,8aR)-6-methyl-3,4a,5,7,8,8a-hexahydro-2H-pyrido[4,3-b][1,4]oxazin-4-yl]pyridazin-3-yl]-5-chloro-3-methyl-phenol;
(322) 2-[6-[(4aS,8aR)-2,3,4a,5,6,7,8,8a-octahydropyrido[4,3-b][1,4]oxazin-4-yl]-4-(difluoromethyl)pyridazin-3-yl]-5-methyl-phenol;
(323) 2-[6-[(4aS,8aR)-6-ethyl-3,4a,5,7,8,8a-hexahydro-2H-pyrido[4,3-b][1,4]oxazin-4-yl]-4-(difluoromethyl)pyridazin-3-yl]-5-methyl-phenol;
(324) 5-[6-[(2S)-2-(hydroxymethyl)morpholin-4-yl]-4-methyl-pyridazin-3-yl]indan-4-ol;
(325) (3R)-3-(hydroxymethyl)-1-[6-[2-hydroxy-6-methyl-4-(trifluoromethyl) phenyl]pyridazin-3-yl]pyrrolidin-2-one;
(326) (3S)-3-(hydroxymethyl)-1-[6-[2-hydroxy-6-methyl-4-(trifluoromethyl) phenyl]pyridazin-3-yl]pyrrolidin-2-one;
(327) 2-[(2R)-4-[6-[2-hydroxy-6-methyl-4-(trifluoromethyl)phenyl]pyridazin-3-yl]morpholin-2-yl]acetonitrile;
(328) 2-[(2S)-4-[6-[2-hydroxy-6-methyl-4-(trifluoromethyl)phenyl]pyridazin-3-yl]morpholin-2-yl]acetonitrile;
(329) (6R)-4-[6-(2-hydroxy-4,6-dimethyl-phenyl)pyridazin-3-yl]-6-(hydroxymethyl)piperazin-2-one;
(330) (6S)-4-[6-(2-hydroxy-4,6-dimethyl-phenyl)pyridazin-3-yl]-6-(hydroxymethyl)piperazin-2-one;
(331) 3-methyl-2-[6-[(2R)-2-[2-(methylamino)ethyl]morpholin-4-yl]pyridazin-3-yl]-5-(trifluoromethyl)phenol;
(332) 3-methyl-2-[6-[(2S)-2-[2-(methylamino)ethyl]morpholin-4-yl]pyridazin-3-yl]-5-(trifluoromethyl)phenol;
(333) 2-[6-[(4aR,7aS)-6-methyl-2,3,4a,5,7,7a-hexahydropyrrolo[3,4-b][1,4]oxazin-4-yl]pyridazin-3-yl]-3-methyl-5-(trifluoromethyl)phenol;
(334) 2-[6-[(4aS,7aR)-6-methyl-2,3,4a,5,7,7a-hexahydropyrrolo[3,4-b][1,4]oxazin-4-yl]pyridazin-3-yl]-3-methyl-5-(trifluoromethyl)phenol;
(335) 1-[rac-(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone;
(336) 1-[(4aR,8aR)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(337) 1-[rac-(4aR,7aR)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone;
(338) 5-chloro-2-[6-[(2R)-2-(fluoromethyl)morpholin-4-yl]pyridazin-3-yl]-3-methyl-phenol;
(339) 5-chloro-2-[6-[(2S)-2-(fluoromethyl)morpholin-4-yl]pyridazin-3-yl]-3-methyl-phenol;
(340) 2-[6-[(rac-3aS,7aR)-6-methyl-3,3a,4,5,7,7a-hexahydro-2H-pyrrolo[2,3-c]pyridin-1-yl]-4-methyl-pyridazin-3-yl]-5-(trifluoromethyl)phenol;
(341) 2-[6-[(rac-3aS,7aR)-6-methyl-3,3a,4,5,7,7a-hexahydro-2H-pyrrolo[2,3-c]pyridin-1-yl]-4-methyl-pyridazin-3-yl]-5-methyl-phenol;
(342) 3,5-dimethyl-2-[6-[rac-(3aS,7aR)-6-methyl-3,3a,4,5,7,7a-hexahydro-2H-pyrrolo[2,3-c]pyridin-1-yl]pyridazin-3-yl]phenol;
(343) 2-[6-[(4aS,8aS)-6-methyl-3,4a,5,7,8,8a-hexahydro-2H-pyrido[4,3-b][1,4]oxazin-4-yl]pyridazin-3-yl]-3-methyl-5-(trifluoromethyl)phenol;
(344) 2-[6-[(4aR,8aR)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol;
(345) 5-[4-(difluoromethyl)-6-[(2S)-2-(hydroxymethyl)morpholin-4-yl]pyridazin-3-yl]indan-4-ol;
(346) 3-methyl-2-[6-[rac-(3aS,6aS)-2,3,3a,5,6,6a-hexahydro-1H-pyrrolo[3,2-b]pyrrol-4-yl]pyridazin-3-yl]-5-(trifluoromethyl)phenol; hydrochloride;
(348) 1-[(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone;
(349) 1-[(4aS,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(350) 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-5-chloro-phenol;
(351) 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-6-fluoro-phenol;
(352) 1-[(4aS,7aR)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone;
(353) 2-[6-[(4aS,8aR)-6-methyl-3,4a,5,7,8,8a-hexahydro-2H-pyrido[4,3-b][1,4]oxazin-4-yl]pyridazin-3-yl]-6-fluoro-3-methyl-phenol;
(354) 2-[6-[(4aS,8aR)-6-methyl-3,4a,5,7,8,8a-hexahydro-2H-pyrido[4,3-b][1,4]oxazin-4-yl]pyridazin-3-yl]-3-methyl-phenol;
(355) 2-[6-[(4aS,8aR)-6-(2,2,2-trifluoroethyl)-3,4a,5,7,8,8a-hexahydro-2H-pyrido[4,3-b][1,4]oxazin-4-yl]pyridazin-3-yl]-3,5-dimethyl-phenol;
(356) 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-6-fluoro-phenol;
(357) 1-[(4aS,8aR)-4-[6-(4-chloro-2-hydroxy-phenyl)-5-methyl-pyridazin-3-yl]-3,4a,5,7,8,8a-hexahydro-2H-pyrido[4,3-b][1,4]oxazin-6-yl]ethanone;
(358) 2-[6-[(4aS,8aR)-6-methyl-3,4a,5,7,8,8a-hexahydro-2H-pyrido[4,3-b][1,4]oxazin-4-yl]pyridazin-3-yl]-3-ethyl-phenol;
(359) 2-[6-[(2S,6R)-2-(hydroxymethyl)-6-methyl-morpholin-4-yl]pyridazin-3-yl]-3,5-dimethyl-phenol;
(360) 2-[6-(5,6-dihydro-2H-pyrazolo[4,3-b][1,4]oxazin-7-yl)pyridazin-3-yl]-3-methyl-5-(trifluoromethyl)phenol;
(361) 3-methyl-2-[6-[rac-(4aR,7aS)-6-ethyl-2,3,4a,5,7,7a-hexahydropyrrolo[3,4-b][1,4]oxazin-4-yl]pyridazin-3-yl]-5-(trifluoromethyl)phenol;
(362) 2-[6-(2,3-dihydropyrido[4,3-b][1,4]oxazin-4-yl)pyridazin-3-yl]-3,5-dimethyl-phenol;
(363) 1-[(4aS,8aR)-4-[6-[2-hydroxy-4-(trifluoromethyl)phenyl]-5-methyl-pyridazin-3-yl]-3,4a,5,7,8,8a-hexahydro-2H-pyrido[4,3-b][1,4]oxazin-6-yl]ethanone;
(364) 2-[6-[rac-(3aS,7aS)-6-methyl-3,3a,4,5,7,7a-hexahydro-2H-pyrrolo[2,3-c]pyridin-1-yl]pyridazin-3-yl]-3-methyl-5-(trifluoromethyl)phenol;
(365) 2-[6-[(2R,6R)-2-(hydroxymethyl)-6-methyl-morpholin-4-yl]pyridazin-3-yl]-3,5-dimethyl-phenol;
(366) 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-(trifluoromethyl)phenol;
(367) 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-(trifluoromethyl)phenol;
(368) 2-(hydroxymethyl)-4-[6-[2-hydroxy-6-methyl-4-(trifluoromethyl)phenyl]pyridazin-3-yl]morpholin-3-one;
(369) 1-[(3aR,7aR)-1-[6-[2-hydroxy-6-methyl-4-(trifluoromethyl)phenyl]pyridazin-3-yl]-3,3a,4,5,7,7a-hexahydro-2H-pyrrolo[2,3-c]pyridin-6-yl]ethanone;
(370) 1-[(3aS,7aS)-1-[6-[2-hydroxy-6-methyl-4-(trifluoromethyl)phenyl]pyridazin-3-yl]-3,3a,4,5,7,7a-hexahydro-2H-pyrrolo[2,3-c]pyridin-6-yl]ethanone;
(371) 2-[6-[rac-(3aS,7aR)-6-methyl-3,3a,4,5,7,7a-hexahydro-2H-pyrrolo[2,3-c]pyridin-1-yl]pyridazin-3-yl]-3-methyl-5-(trifluoromethyl)phenol;
(372) (4S)-1-[5-(difluoromethyl)-6-[2-hydroxy-6-methyl-4-(trifluoromethyl) phenyl]pyridazin-3-yl]-4-hydroxy-pyrrolidin-2-one;
(373) 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-methyl-phenol;
(374) 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-methyl-phenol;
(375) 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-methyl-phenol;
(376) 3-methyl-2-[6-[rac-(4aR,7aR)-6-ethyl-2,3,4a,5,7,7a-hexahydropyrrolo[3,4-b][1,4]oxazin-4-yl]pyridazin-3-yl]-5-(trifluoromethyl)phenol;
(377) 1-[rac-(3aS,6aS)-1-[6-[2-hydroxy-6-methyl-4-(trifluoromethyl)phenyl]pyridazin-3-yl]-2,3,3a,5,6,6a-hexahydropyrrolo[3,2-b]pyrrol-4-yl]ethanone;
(378) 2-[6-[rac-(3aS,7aR)-6-ethyl-3,3a,4,5,7,7a-hexahydro-2H-pyrrolo[2,3-c]pyridin-1-yl]pyridazin-3-yl]-3-methyl-5-(trifluoromethyl)phenol;
(379) 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-chloro-phenol;
(380) 1-[(4aS,8aR)-4-[6-[2-ethyl-6-hydroxy-4-(trifluoromethyl)phenyl]pyridazin-3-yl]-3,4a,5,7,8,8a-hexahydro-2H-pyrido[4,3-b][1,4]oxazin-6-yl]ethanone;
(381) 1-[(4aS,8aR)-4-[6-(4-chloro-2-ethyl-6-hydroxy-phenyl)pyridazin-3-yl]-3,4a,5,7,8,8a-hexahydro-2H-pyrido[4,3-b][1,4]oxazin-6-yl]ethanone;
(382) 2-[6-[(4aS,8aR)-2,3,4a,5,6,7,8,8a-octahydropyrido[4,3-b][1,4]oxazin-4-yl]-4-(difluoromethyl)pyridazin-3-yl]-5-(trifluoromethyl)phenol;
(383) 2-[6-[(3aR,7aS)-6-methyl-3,3a,4,5,7,7a-hexahydro-2H-pyrrolo[2,3-c]pyridin-1-yl]pyridazin-3-yl]-3,5-dimethyl-phenol;
(384) 2-[6-[(3aS,7aR)-6-methyl-3,3a,4,5,7,7a-hexahydro-2H-pyrrolo[2,3-c]pyridin-1-yl]pyridazin-3-yl]-3,5-dimethyl-phenol;
(385) 2-[6-[(3aS,7aR)-6-methyl-3,3a,4,5,7,7a-hexahydro-2H-pyrrolo[2,3-c]pyridin-1-yl]pyridazin-3-yl]-3-methyl-phenol;
(386) 1-[(4aS,8aR)-4-[5-(difluoromethyl)-6-[2-hydroxy-4-(trifluoromethyl) phenyl]pyridazin-3-yl]-3,4a,5,7,8,8a-hexahydro-2H-pyrido[4,3-b][1,4]oxazin-6-yl]ethanone;
(387) 1-[(4aS,8aR)-4-[5-(difluoromethyl)-6-(2-hydroxy-4-methyl-phenyl) pyridazin-3-yl]-3,4a,5,7,8,8a-hexahydro-2H-pyrido[4,3-b][1,4]oxazin-6-yl]ethanone;
(388) 2-[6-[(3aS,7aR)-6-methyl-3,3a,4,5,7,7a-hexahydro-2H-pyrrolo[2,3-c]pyridin-1-yl]pyridazin-3-yl]-3-ethyl-phenol;
(389) 2-[6-[(3aS,7aR)-6-methyl-3,3a,4,5,7,7a-hexahydro-2H-pyrrolo[2,3-c]pyridin-1-yl]pyridazin-3-yl]-3-(trifluoromethyl)phenol;
(390) 2-[6-[(2S,6S)-2-(hydroxymethyl)-6-methyl-morpholin-4-yl]pyridazin-3-yl]-3,5-dimethyl-phenol;
(391) 2-[6-[(3aR,7aS)-6-methyl-3,3a,4,5,7,7a-hexahydro-2H-pyrrolo[2,3-c]pyridin-1-yl]pyridazin-3-yl]-3-methyl-5-(trifluoromethyl)phenol;
(392) 2-[6-[(3aS,7aR)-6-methyl-3,3a,4,5,7,7a-hexahydro-2H-pyrrolo[2,3-c]pyridin-1-yl]pyridazin-3-yl]-3-methyl-5-(trifluoromethyl)phenol;
(393) 2-[6-[(3aR,7aS)-2,3,3a,4,5,6,7,7a-octahydropyrrolo[2,3-c]pyridin-1-yl]pyridazin-3-yl]-3-methyl-5-(trifluoromethyl)phenol;
(394) 1-[(3aR,7aR)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethanone;
(395) 1-[(3aR,7aR)-1-[6-[2-hydroxy-6-methyl-4-(trifluoromethyl)phenyl]pyridazin-3-yl]-3,3a,4,5,7,7a-hexahydro-2H-pyrrolo[2,3-c]pyridin-6-yl]ethanone;
(397) 1-[(4aS,8aR)-4-[6-(4-chloro-2-hydroxy-6-methyl-phenyl)pyridazin-3-yl]-3,4a,5,7,8,8a-hexahydro-2H-pyrido[4,3-b][1,4]oxazin-6-yl]ethanone;
(398) 1-[(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(399) 3,5-dichloro-2-[6-(1,1-dioxo-1,4-thiazinan-4-yl)pyridazin-3-yl]phenol;
(400) 3,5-dichloro-2-[6-(1-oxo-1,4-thiazinan-4-yl)pyridazin-3-yl]phenol;
(402) 2-[6-[(4aS,8aR)-6-methyl-3,4a,5,7,8,8a-hexahydro-2H-pyrido[4,3-b][1,4]oxazin-4-yl]pyridazin-3-yl]-3-(trifluoromethyl)phenol;
(403) 3,5-dimethyl-2-[6-[rac-(4aS,8aR)-3,4a,5,7,8,8a-hexahydro-2H-pyrano[4,3-b][1,4]oxazin-4-yl]pyridazin-3-yl]phenol;
(404) 2-[6-[(4aS,8aR)-6-ethyl-3,4a,5,7,8,8a-hexahydro-2H-pyrido[4,3-b][1,4]oxazin-4-yl]pyridazin-3-yl]-3,5-dimethyl-phenol;
(405) 1-[(4aS,8aR)-4-[4-(difluoromethyl)-6-(2-hydroxy-4,6-dimethyl-phenyl) pyridazin-3-yl]-3,4a,5,7,8,8a-hexahydro-2H-pyrido[4,3-b][1,4]oxazin-6-yl]ethanone;
(406) 2-[6-[(3aS,7aR)-6-ethyl-3,3a,4,5,7,7a-hexahydro-2H-pyrrolo[2,3-c]pyridin-1-yl]pyridazin-3-yl]-3-methyl-5-(trifluoromethyl)phenol;
(407) 2-[6-[(3aS,7aR)-2,3,3a,4,5,6,7,7a-octahydropyrrolo[2,3-c]pyridin-1-yl]pyridazin-3-yl]-3-methyl-5-(trifluoromethyl)phenol;
(408) 2-[6-[(4aS,8aR)-6-ethyl-3,4a,5,7,8,8a-hexahydro-2H-pyrido[4,3-b][1,4]oxazin-4-yl]pyridazin-3-yl]-3-ethyl-phenol;
(409) 1-[(4aS,8aR)-4-[6-(2-hydroxy-6-methyl-phenyl)pyridazin-3-yl]-3,4a,5,7,8,8a-hexahydro-2H-pyrido[4,3-b][1,4]oxazin-6-yl]ethanone;
(410) 3,5-dimethyl-2-[6-(3-methyl-3,6-diazabicyclo[3.2.2]nonan-6-yl)pyridazin-3-yl]phenol;
(411) 2-[6-[(4aS,8aR)-2,3,4a,5,6,7,8,8a-octahydropyrido[4,3-b][1,4]oxazin-4-yl]pyridazin-3-yl]-3-methyl-phenol;
(412) 2-[6-[(4aS,8aR)-6-ethyl-3,4a,5,7,8,8a-hexahydro-2H-pyrido[4,3-b][1,4]oxazin-4-yl]pyridazin-3-yl]-3-methyl-phenol;
(413) 1-[(3aR,7aR)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethanone;
(414) 1-[(3aS,7aS)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethanone;
(415) 2-[6-[(3aS,7aR)-6-methyl-3,3a,4,5,7,7a-hexahydro-2H-pyrrolo[2,3-c]pyridin-1-yl]pyridazin-3-yl]-3-methyl-phenol;
(416) 2-[6-[(3aR,7aS)-6-methyl-3,3a,4,5,7,7a-hexahydro-2H-pyrrolo[2,3-c]pyridin-1-yl]pyridazin-3-yl]-3-methyl-phenol;
(417) 3-methyl-2-[6-(3-methyl-3,6-diazabicyclo[3.2.2]nonan-6-yl)pyridazin-3-yl]-5-(trifluoromethyl)phenol;
(418) 3-ethyl-2-[6-[(2S)-2-(hydroxymethyl)morpholin-4-yl]pyridazin-3-yl]-5-(trifluoromethyl)phenol;
(419) (2S)-2-(hydroxymethyl)-4-[6-[2-hydroxy-6-methyl-4-(trifluoromethyl) phenyl]pyridazin-3-yl]morpholin-3-one;
(420) (2R)-2-(hydroxymethyl)-4-[6-[2-hydroxy-6-methyl-4-(trifluoromethyl) phenyl]pyridazin-3-yl]morpholin-3-one;
(421) 3,5-dimethyl-2-[6-[rac-(4aS,8aS)-3,4a,5,7,8,8a-hexahydro-2H-pyrano[4,3-b][1,4]oxazin-4-yl]pyridazin-3-yl]phenol;
(422) 2-[6-[(3aS,7aR)-6-methyl-3,3a,4,5,7,7a-hexahydro-2H-pyrrolo[2,3-c]pyridin-1-yl]pyridazin-3-yl]-3-ethyl-phenol;
(423) 2-[6-[(3aR,7aS)-6-methyl-3,3a,4,5,7,7a-hexahydro-2H-pyrrolo[2,3-c]pyridin-1-yl]pyridazin-3-yl]-3-ethyl-phenol;
(424) 1-[(4aS,8aR)-4-[6-(2-ethyl-6-hydroxy-phenyl)pyridazin-3-yl]-3,4a,5,7,8,8a-hexahydro-2H-pyrido[4,3-b][1,4]oxazin-6-yl]ethanone;
(425) 2-[6-[(4aS,8aR)-6-methyl-3,4a,5,7,8,8a-hexahydro-2H-pyrido[4,3-b][1,4]oxazin-4-yl]-5-(difluoromethyl)pyridazin-3-yl]-3-ethyl-phenol;
(426) 2-[6-[(3aR,7aS)-6-methyl-3,3a,4,5,7,7a-hexahydro-2H-pyrrolo[2,3-c]pyridin-1-yl]pyridazin-3-yl]-3-(trifluoromethyl)phenol;
(427) 2-[6-[rac-(3aS,7aR)-6-methyl-3,3a,4,5,7,7a-hexahydro-2H-pyrrolo[2,3-c]pyridin-1-yl]pyridazin-3-yl]-3-(trifluoromethyl)phenol;
(428) 3,5-dimethyl-2-[6-(9-methyl-1,9-diazaspiro[4.5]decan-1-yl)pyridazin-3-yl]phenol;
(429) 2-[6-[(3aR,7aS)-6-ethyl-3,3a,4,5,7,7a-hexahydro-2H-pyrrolo[2,3-c]pyridin-1-yl]pyridazin-3-yl]-3-methyl-5-(trifluoromethyl)phenol;
(430) 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-(trifluoromethyl)phenol;
(431) 1-[(4aS,8aR)-4-[6-(2-hydroxy-4-methyl-phenyl)-5-methyl-pyridazin-3-yl]-3,4a,5,7,8,8a-hexahydro-2H-pyrido[4,3-b][1,4]oxazin-6-yl]ethanone;
(432) 2-[6-[(4aS,8aR)-6-isopropyl-3,4a,5,7,8,8a-hexahydro-2H-pyrido[4,3-b][1,4]oxazin-4-yl]pyridazin-3-yl]-3-ethyl-phenol;
(433) 2-[6-[(4aS,8aR)-6-isopropyl-3,4a,5,7,8,8a-hexahydro-2H-pyrido[4,3-b][1,4]oxazin-4-yl]pyridazin-3-yl]-3,5-dimethyl-phenol;
(434) 2-[6-[(4aS,8aR)-6-methyl-3,4a,5,7,8,8a-hexahydro-2H-pyrido[4,3-b][1,4]oxazin-4-yl]-5-methyl-pyridazin-3-yl]-3,5-dimethyl-phenol;
(435) 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-methyl-phenol;
(436) 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-(trifluoromethyl)phenol;
(437) 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-chloro-phenol;
(438) 3-methyl-2-[6-[rac-(4aS,8aR)-3,4a,5,7,8,8a-hexahydro-2H-pyrano[4,3-b][1,4]oxazin-4-yl]pyridazin-3-yl]-5-(trifluoromethyl)phenol;
(439) 2-[6-[(4aS,8aR)-6-methyl-3,4a,5,7,8,8a-hexahydro-2H-pyrido[4,3-b][1,4]oxazin-4-yl]-5-methyl-pyridazin-3-yl]-3-ethyl-phenol;
(440) 3,5-dimethyl-2-[6-(9-methyl-6-oxa-2,9-diazaspiro[4.5]decan-2-yl)pyridazin-3-yl]phenol;
(441) 3,5-dimethyl-2-[6-(1-methyl-4-oxa-1,8-diazaspiro[5.5]undecan-8-yl)pyridazin-3-yl]phenol;
(442) 1-[(4aS,8aR)-4-[6-(4-chloro-2-hydroxy-phenyl)-5-(difluoromethyl) pyridazin-3-yl]-3,4a,5,7,8,8a-hexahydro-2H-pyrido[4,3-b][1,4]oxazin-6-yl]ethanone;
(443) 2-[6-[(4aS,8aR)-6-methyl-3,4a,5,7,8,8a-hexahydro-2H-pyrido[4,3-b][1,4]oxazin-4-yl]-4-ethyl-pyridazin-3-yl]-5-(trifluoromethyl)phenol;
(444) 3,5-dimethyl-2-[6-(6-methyl-9-oxa-2,6-diazaspiro[4.5]decan-2-yl)pyridazin-3-yl]phenol;
(445) 3,5-dimethyl-2-[6-[(5S)-9-methyl-6-oxa-2,9-diazaspiro[4.5]decan-2-yl]pyridazin-3-yl]phenol;
(446) 3,5-dimethyl-2-[6-[(5R)-9-methyl-6-oxa-2,9-diazaspiro[4.5]decan-2-yl]pyridazin-3-yl]phenol;
(448) 3-methyl-2-[6-[rac-(4aS,8aS)-3,4a,5,7,8,8a-hexahydro-2H-pyrano[4,3-b][1,4]oxazin-4-yl]pyridazin-3-yl]-5-(trifluoromethyl)phenol;
(449) 3,5-dimethyl-2-[6-(8-methyl-4-oxa-1,8-diazaspiro[5.5]undecan-1-yl)pyridazin-3-yl]phenol;
(450) 3,5-dimethyl-2-[6-[(5R)-6-methyl-9-oxa-2,6-diazaspiro[4.5]decan-2-yl]pyridazin-3-yl]phenol;
(451) 3,5-dimethyl-2-[6-[(5S)-6-methyl-9-oxa-2,6-diazaspiro[4.5]decan-2-yl]pyridazin-3-yl]phenol;
(452) 2-[6-[rac-(3aS,7aR)-6-methyl-3,3a,4,5,7,7a-hexahydro-2H-pyrrolo[2,3-c]pyridin-1-yl]pyridazin-3-yl]-3-ethyl-5-(trifluoromethyl)phenol;
(455) 3,5-dimethyl-2-[6-(2-methyl-8-oxa-2,5-diazaspiro[3.5]nonan-5-yl)pyridazin-3-yl]phenol;
(456) 2-[6-[(4aS,8aR)-6-ethyl-3,4a,5,7,8,8a-hexahydro-2H-pyrido[4,3-b][1,4]oxazin-4-yl]-4-(difluoromethyl)pyridazin-3-yl]-5-chloro-phenol;
(457) 1-[(3aR,7aR)-1-[6-(2-hydroxy-4-methyl-phenyl)-5-methyl-pyridazin-3-yl]-3,3a,4,5,7,7a-hexahydro-2H-pyrrolo[2,3-c]pyridin-6-yl]ethanone;
(458) 2-[6-[rac-(3aS,7aR)-6-ethyl-3,3a,4,5,7,7a-hexahydro-2H-pyrrolo[2,3-c]pyridin-1-yl]pyridazin-3-yl]-3-(trifluoromethyl)phenol;
(459) 2-[6-[rac-(3aS,7aR)-6-methyl-3,3a,4,5,7,7a-hexahydro-2H-pyrrolo[2,3-c]pyridin-1-yl]pyridazin-3-yl]-3-ethyl-5-methyl-phenol;
(460) 2-[6-[rac-(3aS,7aR)-6-ethyl-3,3a,4,5,7,7a-hexahydro-2H-pyrrolo[2,3-c]pyridin-1-yl]-4-methyl-pyridazin-3-yl]-5-methyl-phenol;
(461) 2-[6-[(4aS,8aR)-6-methyl-3,4a,5,7,8,8a-hexahydro-2H-pyrido[4,3-b][1,4]oxazin-4-yl]pyridazin-3-yl]-5-chloro-3-ethyl-phenol;
(462) 1-[(4aR,8aR)-5-[6-[2-hydroxy-6-methyl-4-(trifluoromethyl)phenyl]pyridazin-3-yl]-2,3,4,4a,6,7,8,8a-octahydro-1,5-naphthyridin-1-yl]ethanone;
(463) 1-[(4aS,8aS)-5-[6-[2-hydroxy-6-methyl-4-(trifluoromethyl)phenyl]pyridazin-3-yl]-2,3,4,4a,6,7,8,8a-octahydro-1,5-naphthyridin-1-yl]ethanone;
(466) (4aS,6S,7aR)-4-[6-(2-hydroxy-4,6-dimethyl-phenyl)pyridazin-3-yl]-3,4a,5,6,7,7a-hexahydro-2H-cyclopenta[b][1,4]oxazin-6-ol;
(467) (4aR,6R,7aS)-4-[6-(2-hydroxy-4,6-dimethyl-phenyl)pyridazin-3-yl]-3,4a,5,6,7,7a-hexahydro-2H-cyclopenta[b][1,4]oxazin-6-ol;
(468) 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-chloro-phenol;
(469) 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-chloro-phenol;
(470) 2-[6-[(4aS,8aR)-6-methyl-3,4a,5,7,8,8a-hexahydro-2H-pyrido[4,3-b][1,4]oxazin-4-yl]-4-ethyl-pyridazin-3-yl]-5-methyl-phenol;
(471) 5-[6-[(4aS,8aR)-6-methyl-3,4a,5,7,8,8a-hexahydro-2H-pyrido[4,3-b][1,4]oxazin-4-yl]-4-methyl-pyridazin-3-yl]indan-4-ol;
(472) 2-[6-[(4aS,8aR)-6-isopropyl-3,4a,5,7,8,8a-hexahydro-2H-pyrido[4,3-b][1,4]oxazin-4-yl]-4-methyl-pyridazin-3-yl]-5-methyl-phenol;
(473) 2-[6-[(3aS,7aR)-6-ethyl-3,3a,4,5,7,7a-hexahydro-2H-pyrrolo[2,3-c]pyridin-1-yl]pyridazin-3-yl]-3-(trifluoromethyl)phenol;
(474) 2-[6-[(3aR,7aS)-6-ethyl-3,3a,4,5,7,7a-hexahydro-2H-pyrrolo[2,3-c]pyridin-1-yl]-4-methyl-pyridazin-3-yl]-5-methyl-phenol;
(475) 2-[6-[(3aS,7aR)-6-ethyl-3,3a,4,5,7,7a-hexahydro-2H-pyrrolo[2,3-c]pyridin-1-yl]-4-methyl-pyridazin-3-yl]-5-methyl-phenol;
(476) 2-[6-[(3aR,7aS)-6-methyl-3,3a,4,5,7,7a-hexahydro-2H-pyrrolo[2,3-c]pyridin-1-yl]pyridazin-3-yl]-3-ethyl-5-methyl-phenol;
(477) 2-[6-[(3aS,7aR)-6-methyl-3,3a,4,5,7,7a-hexahydro-2H-pyrrolo[2,3-c]pyridin-1-yl]pyridazin-3-yl]-3-ethyl-5-methyl-phenol;
(479) 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-fluoro-phenol;
(480) 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-fluoro-phenol;
(481) 2-[6-[(4aS,8aR)-6-methyl-3,4a,5,7,8,8a-hexahydro-2H-pyrido[4,3-b][1,4]oxazin-4-yl]pyridazin-3-yl]-3-ethyl-5-(trifluoromethyl)phenol;
(482) 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-(trifluoromethyl)phenol;
(483) 2-[6-[(3aS,7aR)-6-methyl-3,3a,4,5,7,7a-hexahydro-2H-pyrrolo[2,3-c]pyridin-1-yl]-4-(difluoromethyl)pyridazin-3-yl]-5-(trifluoromethyl)phenol;
(484) 3-methyl-2-[6-[rac-(3aS,6aS)-1-methyl-2,3,3a,5,6,6a-hexahydropyrrolo[3,2-b]pyrrol-4-yl]pyridazin-3-yl]-5-(trifluoromethyl)phenol;
(485) 2-[6-[(3aR,7aS)-6-ethyl-3,3a,4,5,7,7a-hexahydro-2H-pyrrolo[2,3-c]pyridin-1-yl]-4-(difluoromethyl)pyridazin-3-yl]-5-(trifluoromethyl)phenol;
(486) 2-[6-[(3aS,7aR)-6-ethyl-3,3a,4,5,7,7a-hexahydro-2H-pyrrolo[2,3-c]pyridin-1-yl]-4-(difluoromethyl)pyridazin-3-yl]-5-(trifluoromethyl)phenol;
(487) 2-[6-[(3aR,7aS)-6-methyl-3,3a,4,5,7,7a-hexahydro-2H-pyrrolo[2,3-c]pyridin-1-yl]-5-(difluoromethyl)pyridazin-3-yl]-3,5-dimethyl-phenol;
(488) 2-[6-[(3aS,7aR)-6-methyl-3,3a,4,5,7,7a-hexahydro-2H-pyrrolo[2,3-c]pyridin-1-yl]-5-(difluoromethyl)pyridazin-3-yl]-3,5-dimethyl-phenol;
(489) 2-[6-[(3aR,7aS)-6-ethyl-3,3a,4,5,7,7a-hexahydro-2H-pyrrolo[2,3-c]pyridin-1-yl]-4-(difluoromethyl)pyridazin-3-yl]-5-methyl-phenol;
(490) 2-[6-[(3aS,7aR)-6-ethyl-3,3a,4,5,7,7a-hexahydro-2H-pyrrolo[2,3-c]pyridin-1-yl]-4-(difluoromethyl)pyridazin-3-yl]-5-methyl-phenol;
(491) 2-[6-[(3aR,7aS)-6-methyl-3,3a,4,5,7,7a-hexahydro-2H-pyrrolo[2,3-c]pyridin-1-yl]pyridazin-3-yl]-3-ethyl-5-(trifluoromethyl)phenol;
(492) 2-[6-[(3aS,7aR)-6-methyl-3,3a,4,5,7,7a-hexahydro-2H-pyrrolo[2,3-c]pyridin-1-yl]pyridazin-3-yl]-3-ethyl-5-(trifluoromethyl)phenol;
(493) 2-[6-[(3aR,7aS)-6-methyl-3,3a,4,5,7,7a-hexahydro-2H-pyrrolo[2,3-c]pyridin-1-yl]pyridazin-3-yl]-5-chloro-3-ethyl-phenol
(494) 2-[6-[(3aS,7aR)-6-methyl-3,3a,4,5,7,7a-hexahydro-2H-pyrrolo[2,3-c]pyridin-1-yl]pyridazin-3-yl]-5-chloro-3-ethyl-phenol;
(496) 2-[6-[(3aR,7aS)-6-ethyl-3,3a,4,5,7,7a-hexahydro-2H-pyrrolo[2,3-c]pyridin-1-yl]pyridazin-3-yl]-5-chloro-3-methyl-phenol;
(497) 2-[6-[(3aS,7aR)-6-ethyl-3,3a,4,5,7,7a-hexahydro-2H-pyrrolo[2,3-c]pyridin-1-yl]pyridazin-3-yl]-5-chloro-3-methyl-phenol;
(498) 2-[6-[(3aS,7aR)-6-ethyl-3,3a,4,5,7,7a-hexahydro-2H-pyrrolo[2,3-c]pyridin-1-yl]pyridazin-3-yl]-3-methyl-phenol;
(499) 2-[6-[(3aR,7aS)-6-ethyl-3,3a,4,5,7,7a-hexahydro-2H-pyrrolo[2,3-c]pyridin-1-yl]pyridazin-3-yl]-3-methyl-phenol;
(500) 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-chloro-phenol;
(501) 2-[6-[(3aS,7aR)-6-methyl-3,3a,4,5,7,7a-hexahydro-2H-pyrrolo[2,3-c]pyridin-1-yl]-4-(difluoromethyl)pyridazin-3-yl]-5-chloro-phenol;
(502) 2-[6-[(3aS,7aR)-6-methyl-3,3a,4,5,7,7a-hexahydro-2H-pyrrolo[2,3-c]pyridin-1-yl]pyridazin-3-yl]-5-chloro-3-methyl-phenol;
(503) 2-[6-[(3aR,7aS)-6-methyl-3,3a,4,5,7,7a-hexahydro-2H-pyrrolo[2,3-c]pyridin-1-yl]pyridazin-3-yl]-5-chloro-3-methyl-phenol;
(504) 2-[6-[(3aS,7aR)-6-ethyl-3,3a,4,5,7,7a-hexahydro-2H-pyrrolo[2,3-c]pyridin-1-yl]pyridazin-3-yl]-3,5-dimethyl-phenol;
(505) 2-[6-[(3aR,7aS)-6-ethyl-3,3a,4,5,7,7a-hexahydro-2H-pyrrolo[2,3-c]pyridin-1-yl]pyridazin-3-yl]-3,5-dimethyl-phenol;
(506) 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-methyl-phenol;
(507) 2-[6-[(3aS,7aR)-6-methyl-3,3a,4,5,7,7a-hexahydro-2H-pyrrolo[2,3-c]pyridin-1-yl]-4-(difluoromethyl)pyridazin-3-yl]-5-methyl-phenol;
(508) 5-ethynyl-2-[6-[(2S)-2-(hydroxymethyl)morpholin-4-yl]pyridazin-3-yl]-3-methyl-phenol;
(509) 2-[6-[(2S)-2-(hydroxymethyl)morpholin-4-yl]pyridazin-3-yl]-5-methyl-3-(trifluoromethyl)phenol;
(510) 1-[(3aS,7aS)-1-[5-(difluoromethyl)-6-[2-hydroxy-4-(trifluoromethyl) phenyl]pyridazin-3-yl]-3,3a,4,5,7,7a-hexahydro-2H-pyrrolo[2,3-c]pyridin-6-yl]ethanone;
(511) 1-[(3aR,7aR)-1-[5-(difluoromethyl)-6-[2-hydroxy-4-(trifluoromethyl) phenyl]pyridazin-3-yl]-3,3a,4,5,7,7a-hexahydro-2H-pyrrolo[2,3-c]pyridin-6-yl]ethanone;
(512) 1-[(3aS,7aS)-1-[4-(difluoromethyl)-6-(2-hydroxy-4,6-dimethyl-phenyl)pyridazin-3-yl]-3,3a,4,5,7,7a-hexahydro-2H-pyrrolo[2,3-c]pyridin-6-yl]ethanone;
(513) 1-[(3aR,7aR)-1-[4-(difluoromethyl)-6-(2-hydroxy-4,6-dimethyl-phenyl)pyridazin-3-yl]-3,3a,4,5,7,7a-hexahydro-2H-pyrrolo[2,3-c]pyridin-6-yl]ethanone;
(514) 1-[(3aR,7aR)-1-[6-[2-hydroxy-4-(trifluoromethyl)phenyl]-5-methyl-pyridazin-3-yl]-3,3a,4,5,7,7a-hexahydro-2H-pyrrolo[2,3-c]pyridin-6-yl]ethanone;
(515) 2-[6-[(4aS,8aS)-5-methyl-2,3,4,4a,6,7,8,8a-octahydro-1,5-naphthyridin-1-yl]pyridazin-3-yl]-3-methyl-5-(trifluoromethyl)phenol;
(516) 2-[6-[(4aR,8aR)-5-methyl-2,3,4,4a,6,7,8,8a-octahydro-1,5-naphthyridin-1-yl]pyridazin-3-yl]-3-methyl-5-(trifluoromethyl)phenol;
(517) 2-[6-[(4aR,8aS)-7-methyl-2,3,4,4a,5,6,8,8a-octahydro-1,7-naphthyridin-1-yl]pyridazin-3-yl]-3,5-dimethyl-phenol;
(518) 2-[6-[(4aS,8aS)-7-methyl-2,3,4,4a,5,6,8,8a-octahydro-1,7-naphthyridin-1-yl]pyridazin-3-yl]-3,5-dimethyl-phenol;
(519) 2-[6-[(4aR,8aR)-7-methyl-2,3,4,4a,5,6,8,8a-octahydro-1,7-naphthyridin-1-yl]pyridazin-3-yl]-3,5-dimethyl-phenol;
(520) 2-[6-[(4aS,8aR)-7-methyl-2,3,4,4a,5,6,8,8a-octahydro-1,7-naphthyridin-1-yl]pyridazin-3-yl]-3,5-dimethyl-phenol;
(521) 2-[6-[(4aS,8aR)-6-(2-fluoroethyl)-3,4a,5,7,8,8a-hexahydro-2H-pyrido[4,3-b][1,4]oxazin-4-yl]pyridazin-3-yl]-3,5-dimethyl-phenol;
(522) 2-[6-[(4aS,8aR)-6-(2-fluoroethyl)-3,4a,5,7,8,8a-hexahydro-2H-pyrido[4,3-b][1,4]oxazin-4-yl]pyridazin-3-yl]-5-chloro-3-methyl-phenol;
(523) 2-[6-[(4aR,8aR)-7-methyl-2,3,4,4a,5,6,8,8a-octahydro-1,7-naphthyridin-1-yl]pyridazin-3-yl]-3-methyl-5-(trifluoromethyl)phenol;
(524) 2-[6-[(4aS,8aS)-7-methyl-2,3,4,4a,5,6,8,8a-octahydro-1,7-naphthyridin-1-yl]pyridazin-3-yl]-3-methyl-5-(trifluoromethyl)phenol;
(525) 2-[6-[(4aS,8aR)-6-(2-fluoroethyl)-3,4a,5,7,8,8a-hexahydro-2H-pyrido[4,3-b][1,4]oxazin-4-yl]pyridazin-3-yl]-5-chloro-phenol;
(526) 2-[6-[(4aS,8aR)-6-ethyl-3,4a,5,7,8,8a-hexahydro-2H-pyrido[4,3-b][1,4]oxazin-4-yl]pyridazin-3-yl]-5-chloro-3-ethyl-phenol;
(527) 2-[6-[(4aS,8aR)-6-(3-fluoropropyl)-3,4a,5,7,8,8a-hexahydro-2H-pyrido[4,3-b][1,4]oxazin-4-yl]pyridazin-3-yl]-3,5-dimethyl-phenol; and
(528) 5-methyl-2-[4-methyl-6-[rac-(3aS,7aR)-6-isopropyl-3,3a,4,5,7,7a-hexahydro-2H-pyrrolo[2,3-c]pyridin-1-yl]pyridazin-3-yl]phenol.

Among the preceding listed compounds of formula (I) or a pharmaceutically acceptable salt thereof, the following compounds which are of interest may for example be cited: 1, 2, 5, 6, 8, 9, 14 to 31, 33 to 36, 38 to 41, 43 to 45, 48 to 54, 56 to 63, 65, 67 to 82, 85 to 90, 92, 93, 96 to 99, 102, 103, 105 to 110, 112, 113, 115 to 119, 121 to 124, 126 to 153, 155 to 161, 163 to 167, 169 to 174, 177 to 184, 186 to 189, 191, 192, 194, 198 to 201, 203, 204, 209 to 212, 216, 219, 220, 233 to 235, 237, 238, 245 to 250, 258, 259, 266, 267, 273 to 280, 283 to 288, 292, 293, 296, 297, 302, 303, 305, 324 to 332, 338, 339, 345, 359, 365, 368, 372, 390, 399, 400, 418 to 420, 508, and 509.

### Preparation of the Compounds of Formula (I)

The compounds of formula (I) or a pharmaceutically acceptable salt thereof according to the present disclosure may be prepared according to various methods. More particularly, they can be prepared as illustrated by the schemes which follow.

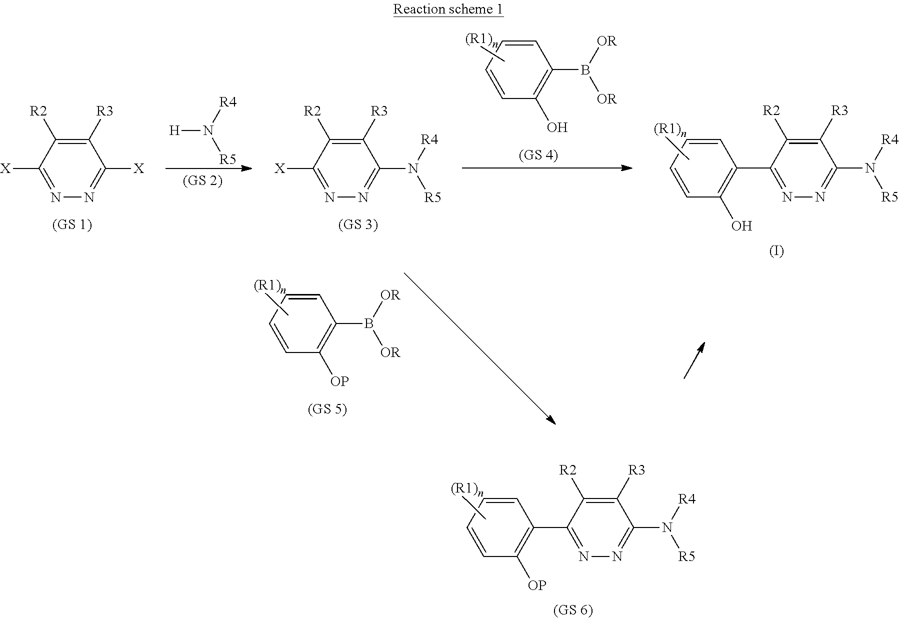

In the scheme 1 above, R1, n, R2, R3, R4 and R5 are as defined in the present disclosure, and X represent independently of each other a halogen atom, such as Cl, Br or I, P represents a protecting group like a methyl group, a paramethoxybenzyl group, a benzyl group, a methoxymethyl group or an ethoxyethyl group and B(OR)₂ represents either a boronic acid or a boronic ester, e.g. 4,4,5,5-tetramethyl-1,3,2-dioxaborolan.

Compounds of the invention may be prepared by the reaction scheme I where a dilhalogenopyridazine properly substituted (GS 1) is reacted with an appropriate amine (GS 2) in a presence of a base (like K₂CO₃, DIPEA), at room temperature or elevated temperature (preferentially between 120-180° C.) to give halogenopyridazine (GS 3). This reaction may be carried out in sealed tubes, and also under microwave irradiation. This reaction can be also realized by metal coupling, using Pd catalyst (like Pd(dppf)Cl₂) and a mineral base like K₃PO₄, at elevated temperature (typically between 100-110° C.), in a non protic solvent like 1,4-dioxane to provide a compound of general formula (I) according to the present disclosure. Then, this intermediate is reacted with an appropriate phenyl boronate derivative (GS 4), where it could be under a form of boronic acid or a boronate (like 4,4,5,5-tetramethyl-1,3,2-dioxaborolan), in a Suzuki type reaction, in the presence of a Pd catalyst (like Pd(PPh₃)₄, or Pd(dppf)Cl₂) and a mineral base like NaHCO₃, at elevated temperature (typically between 100-140° C.), in a mix of solvent like 1,4-dioxane/Water or DME/water to provide a compound of general formula (I). It is to note that phenyl boronate derivative could be used also with a protected hydroxy function, with a methyl, or benzyl for example (GS 5), to give the appropriate phenyl pyridazine (GS 6), which is submitted to deprotection conditions (BBr₃, or hydrogenation for example) to provide compound of general formula (I) according to the present disclosure.

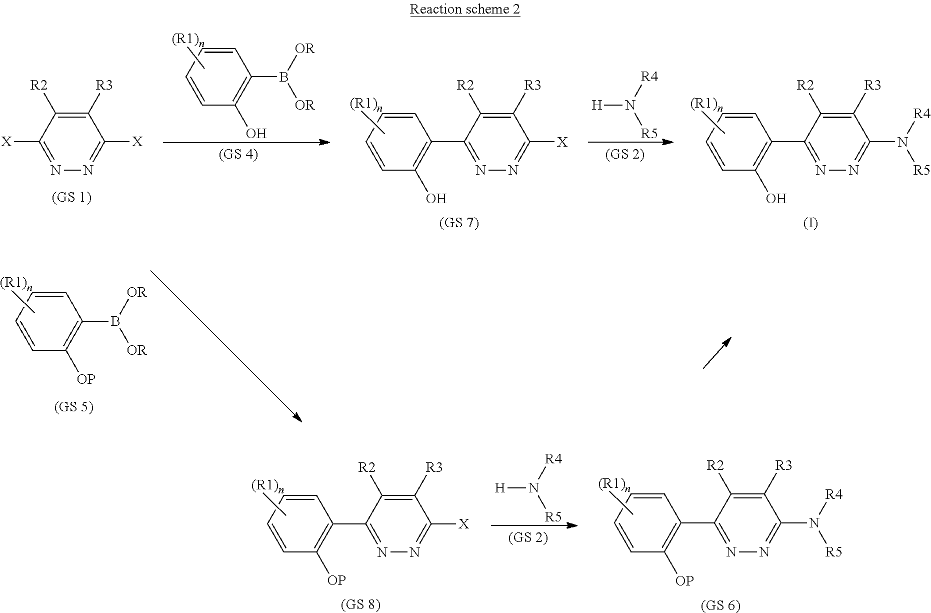

In the scheme 2 above, R1, n, R2, R3, R4 and R5 are as defined in the present disclosure, and X represent independently of each other a halogen atom, such as Cl, Br or I, P represents a protecting group like a methyl group, a paramethoxybenzyl group, a benzyl group, a methoxymethyl group or an ethoxyethyl group and B(OR)₂ represents either a boronic acid or a boronic ester, e.g. 4,4,5,5-tetramethyl-1,3,2-dioxaborolan.

Compounds of the invention may also be prepared by the reaction scheme 2 where a dihalogenopyridazine properly substituted (GS 1) is reacted with an appropriate phenyl boronate derivative (GS 4), where it could be under a form of boronic acid or a boronate like 4,4,5,5-tetramethyl-1,3,2-dioxaborolan, in a Suzuki type reaction, in the presence of a Pd catalyst (like Pd(PPh₃)₄ or Pd(dppf)Cl₂) and a mineral base like Na₂CO₃, in a mix of solvent like 1,4-dioxane/water or DME/water, at a temperature between 10° and 140° C., to give halogenopyridazine (GS 7). Then, this intermediate is reacted with an appropriate amine (GS 2) in a presence of a base (like NaHCO₃, DIPEA), at elevated temperature (preferentially between 130-180° C.), also possibly carried out in sealed tubes and also under microwave irradiation, to provide a compound of general formula (I) according to the present disclosure. The suzuki reaction could also be carried out using a phenyl boronate derivative with a protected alcohol function (methyl for example) (GS 5) using similar conditions to give the appropriate halogenopyridazine (GS 8), which gives after N-arylation using an appropriate amine (GS 2) in similar conditions as previous, and then submitted to deprotection conditions (BBr₃ for example) to provide compound of general formula (I) according to the present disclosure.

The intermediate halogenopyridazine (GS 7) may also be prepared by the reaction scheme 3 as shown below.

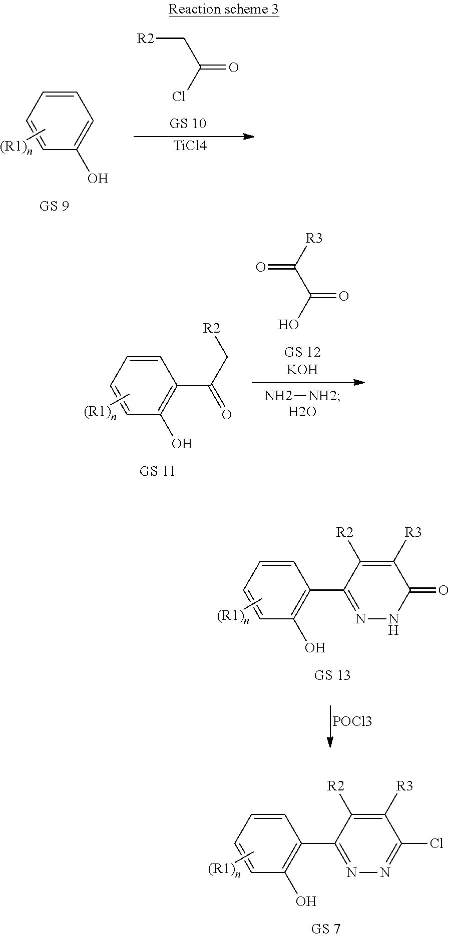

In case of n is 1, R1 represents a (C₁-C₄)alkyl group, and P2 and P3 represent independently of each other a hydrogen atom or a (C₁-C₄)alkyl group, (GS 7) may also be prepared by the reaction scheme 3 where a phenol properly substituted (GS 9) is reacted in a Friedel-Crafts acylation with an appropriate alkanoyl chloride (GS 10) in a presence of a Lewis acid such as TiCl₄ (Titanium (IV) chloride), at elevated temperature (for example 120° C.) to give the acylated phenol GS 11. This intermediate is reacted with an a-oxo acid (GS 12) in a basic media (for example KOH (potassium hydroxide)), then after treatment, in presence of hydrazine hydrate (NH₂—NH₂, H₂O) at elevated temperature (for example 10° C.) to give the pyridazone (GS 13). Then, to the intermediate (GS 13) is added a chlorinated agent like POCl₃ (phosphoryl chloride), in a polar solvent, for example DMF, at 80-90° C. to give the chloropyridazine derivative (GS 7).

The specific compounds of formula (I) or a pharmaceutically acceptable salt thereof as defined in the present disclosure are indicated in Table 1 (number, and formula) and are further detailed hereafter. In Table 1, ¹H NMR, and liquid chromatography/mass spectra are also indicated.

The ¹H NMR of Table 1 is ¹H NMR Spectra (400 MHz or 500 MHz, δ in ppm, DMSO-d6, CD₃OD or CDCl₃) as defined in the Experimental part.

The liquid chromatography/mass spectra (LC/MS) of Table 1 were obtained according to one of the six methods described in the Experimental part.

TABLE 1

| | | MS (m/z, | |
|---|---|---|---|
| | | [M + H]⁺) | |
| | | Rt (min) | |
| | | Analytical | |
| Ex | Compounds of formula (I) | method | ¹H NMR |
| 1 | 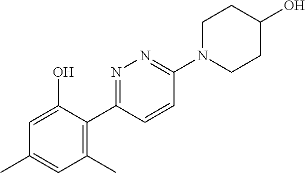 [SMILES: Cc1cc(C)c(-c2ccc(N3CCC(O)CC3)nn2)c(O)c1] | 300.1 0.55 A | 1H NMR (400 MHz, DMSO-d6, 30° C.) δ ppm 1.42 (dtd, J = 12.9, 9.3, 9.3, 3.8 Hz, 2 H), 1.76-1.93 (m, 2 H), 2.04 (s, 3 H), 2.22 (s, 3 H), 3.21 (ddd, J = 13.2, 10.1, 3.1 Hz, 2 H), 3.75 (tq, J = 8.5, 4.1 Hz, 1 H), 4.09 (dt, J = 13.3, 4.2 Hz, 2 H), 4.71 (d, J = 4.3 Hz, 1 H), 6.57 (br s, 1 H), 6.58 (br s, 1 H), 7.21-7.36 (m, 2 H), 9.40 (s, 1 H) |
| 2 | 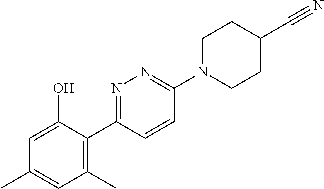 [SMILES: Cc1cc(C)c(-c2ccc(N3CCC(C#N)CC3)nn2)c(O)c1] | 309.2 1.05 B | 1H NMR (400 MHz, DMSO-d6, 30° C.) δ ppm 1.70- 1.85 (m, 2 H), 1.93-2.03 (m, 2 H), 2.03 (s, 3 H), 2.22 (s, 3 H), 3.10-3.21 (m, 1 H), 3.42-3.56 (m, 2 H), 3.86-4.00 (m, 2 H), 6.56-6.57 (br s, 1 H), 6.57- 6.59 (br s, 1 H), 7.23-7.42 (m, 2 H), 9.37 (s, 1 H) |
| 3 | 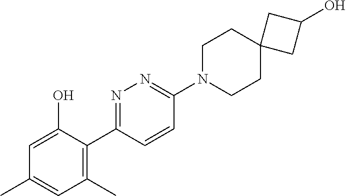 [SMILES: Cc1cc(C)c(-c2ccc(N3CCC4(CC3)CC(O)C4)nn2)c(O)c1] | 340.2 1.00 B | 1H NMR (400 MHz, DMSO-d6, 30° C.) δ ppm 1.58- 1.71 (m, 6 H), 2.09 (s, 3 H), 2.16-2.25 (m, 2 H), 2.24 (s, 3 H), 3.62 (dt, J = 19.2, 5.4 Hz, 4 H), 4.10- 4.23 (m, 1 H), 4.68-5.21 (m, 1 H), 6.63 (s, 2 H), 7.67-7.95 (m, 2 H), 9.49-10.33 (m, 1 H) |
| 4 | 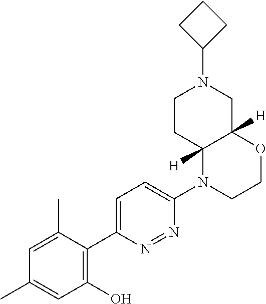 [SMILES: Cc1cc(C)c(-c2ccc(N3CCO[C@H]4CN(C5CCC5)CC[C@H]43)nn2)c(O)c1] | 395 0.395 C | 1H NMR (400 MHz, CD3OD) δ ppm 7.47 (d, J = 9.4 Hz, 1H), 7.31 (d, J = 9.6 Hz, 1H), 6.64 (s, 1H), 6.59 (s, 1H), 4.67 (br d, J = 11.2 Hz, 1H), 4.21 (dd, J = 3.2, 11.4 Hz, 1H), 3.98 (br s, 1H), 3.94-3.79 (m, 2H), 3.54-3.43 (m, 2H), 3.35 (br dd, J = 4.0, 12.4 Hz, 2H), 3.02-2.91 (m, 1H), 2.79-2.67 (m, 1H), 2.37-2.20 (m, 7H), 2.18-2.11 (m, 1H), 2.08 (s, 3H), 1.96-1.77 (m, 3H) |
| 5 | 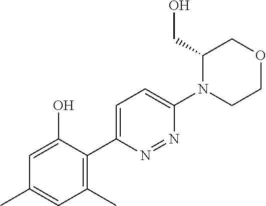 [SMILES: Cc1cc(C)c(-c2ccc(N3CCOC[C@H]3CO)nn2)c(O)c1] | 316.3 0.91 B | 1H NMR (400 MHz, DMSO-d6, 30° C.) δ ppm 2.04 (s, 3 H), 2.22 (s, 3 H), 3.15 (td, J = 12.8, 3.9 Hz, 1 H), 3.43-3.64 (m, 3 H), 3.69-3.81 (m, 1 H), 3.95 (dd, J = 11.3, 3.6 Hz, 1 H), 4.04-4.23 (m, 3 H), 4.85 (dd, J = 6.2, 4.9 Hz, 1 H), 6.56-6.57 (m, 1 H), 6.57-6.59 (m, 1 H), 7.19 (d, J = 9.5 Hz, 1 H), 7.32 (d, J = 9.5 Hz, 1 H), 9.39 (s, 1 H) |
| 6 | 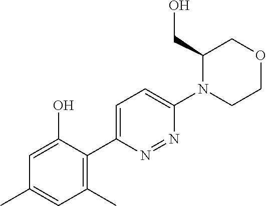 [SMILES: Cc1cc(C)c(-c2ccc(N3CCOC[C@@H]3CO)nn2)c(O)c1] | 316.3 0.91 B | 1H NMR (400 MHz, DMSO-d6, 30° C.) δ ppm 2.04 (s, 3 H), 2.22 (s, 3 H), 3.15 (td, J = 12.7, 3.6 Hz, 1 H), 3.44-3.62 (m, 3 H), 3.74 (td, J = 9.6, 6.4 Hz, 1 H), 3.95 (br dd, J = 11.3, 3.1 Hz, 1 H), 4.00-4.26 (m, 3 H), 4.87 (t, J = 5.5 Hz, 1 H), 6.57 (br s, 1 H), 6.58 (br s, 1 H), 7.20 (d, J = 9.5 Hz, 1 H), 7.32 (d, J = 9.5 Hz, 1 H), 9.41 (s, 1 H) |
| 7 | 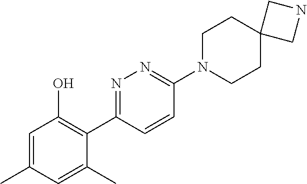 [SMILES: Cc1cc(C)c(-c2ccc(N3CCC4(CC3)CNC4)nn2)c(O)c1] | 325.3 0.38 A | 1H NMR (400 MHz, DMSO-d6, 30° C.) δ ppm 1.86- 1.94 (m, 4 H), 2.08 (s, 3 H), 2.25 (s, 3 H), 3.65-3.71 (m partially hidden, 4 H), 3.81 (t, J = 6.3 Hz, 4 H), 6.64 (s, 2 H), 7.69-7.96 (m, 2 H), 8.67-8.90 (m, 2 H), 9.55-10.23 (m, 1 H) |
| 8 | 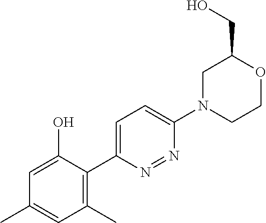 [SMILES: Cc1cc(C)c(-c2ccc(N3CCO[C@H](CO)C3)nn2)c(O)c1] | 316.2 0.92 B | 1H NMR (400 MHz, DMSO-d6, 30° C.) δ ppm 2.04 (s, 3 H), 2.23 (s, 3 H), 2.71 (dd, J = 12.8, 9.9 Hz, 1 H), 2.97 (td, J = 12.3, 3.5 Hz, 1 H), 3.41-3.58 (m, 3 H), 3.62 (td, J = 11.6, 2.6 Hz, 1 H), 4.00 (dd, J = 11.5, 2.3 Hz, 1 H), 4.13 (br d, J = 12.9 Hz, 1 H), 4.32 (br d, J = 12.6 Hz, 1 H), 4.82 (t, J = 5.5 Hz, 1 H), 6.58 (br s, 1 H), 6.59 (br s, 1 H), 7.27 (d, J = 9.5 Hz, 1 H), 7.35 (d, J = 9.5 Hz, 1 H), 9.39 (s, 1 H) |
| 9 | 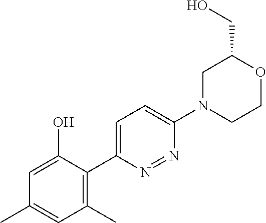 [SMILES: Cc1cc(C)c(-c2ccc(N3CCO[C@@H](CO)C3)nn2)c(O)c1] | 316.3 0.93 B | 1H NMR (400 MHz, DMSO-d6, 30° C.) δ ppm 2.03 (s, 3 H) 2.22 (s, 3 H) 2.71 (dd, J = 12.7, 9.8 Hz, 1 H) 2.97 (td, J = 12.3, 3.4 Hz, 1 H) 3.41-3.56 (m, 3 H) 3.61 (td, J = 11.6, 2.7 Hz, 1 H) 3.99 (dd, J = 11.5, 2.2 Hz, 1 H) 4.12 (br d, J = 12.8 Hz, 1 H) 4.31 (br d, J = 12.7 Hz, 1 H) 4.82 (br s, 1 H) 6.53-6.64 (m, 2 H) 7.27 (d, J = 9.5 Hz, 1 H) 7.34 (d, J = 9.5 Hz, 1 H) 9.25-9.47 (m, 1 H) |
| 10 | 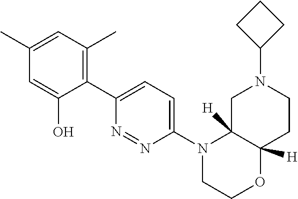 [SMILES: Cc1cc(C)c(-c2ccc(N3CCO[C@H]4CCN(C5CCC5)C[C@H]43)nn2)c(O)c1] | 395 0.421 C | 1H NMR (400 MHz, CD3OD) δ ppm 7.45 (d, J = 9.6 Hz, 1H), 7.26 (d, J = 9.6 Hz, 1H), 6.65 (s, 1H), 6.60 (s, 1H), 4.47 (br d, J = 10.6 Hz, 1H), 4.12 (dd, J = 3.6, 11.6 Hz, 1H), 4.06-3.96 (m, 1H), 3.87 (br d, J = 2.6 Hz, 1H), 3.78 (dt, J = 3.4, 11.8 Hz, 1H), 3.31- 3.25 (m, 1H), 2.95-2.85 (m, 1H), 2.79-2.65 (m, 2H), 2.40 (br t, J = 11.0 Hz, 1H), 2.29 (s, 3H), 2.21- 2.02 (m, 6H), 2.00-1.85 (m, 4H), 1.80-1.66 (m, 2H) |
| 11 | 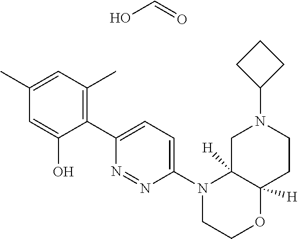 [SMILES: O=CO ; Cc1cc(C)c(-c2ccc(N3CCO[C@@H]4CCN(C5CCC5)C[C@@H]43)nn2)c(O)c1] | 395 0.419 C | 1H NMR (400 MHz, DMSO-d6) δ ppm 9.53-9.26 (m, 1H), 8.23 (br d, J = 1.2 Hz, 1H), 7.33 (d, J = 9.6 Hz, 1H), 7.19 (d, J = 9.6 Hz, 1H), 6.57 (br d, J = 5.4 Hz, 2H), 4.35 (br d, J = 10.0 Hz, 1H), 4.08-4.00 (m, 1H), 3.94 (br d, J = 12.6 Hz, 1H), 3.74 (br s, 1H), 3.65-3.58 (m, 1H), 3.14 (dt, J = 3.4, 12.6 Hz, 1H), 2.74 (quin, J = 7.6 Hz, 1H), 2.54 (br d, J = 4.6 Hz, 2H), 2.26-2.14 (m, 4H), 2.02 (s, 3H), 1.98-1.84 (m, 3H), 1.84-1.65 (m, 4H), 1.59 (br d, J = 6.4 Hz, 2H) |
| 12 | 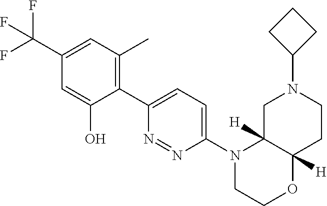 [SMILES: Cc1cc(C(F)(F)F)cc(O)c1-c1ccc(N2CCO[C@H]3CCN(C4CCC4)C[C@H]32)nn1] | 449 0.48 D | 1H NMR (400 MHz, CD3OD) δ ppm 7.47 (d, J = 9.6 Hz, 1H), 7.28 (d, J = 9.6 Hz, 1H), 7.09 (s, 1H), 7.02 (s, 1H), 4.55-4.45 (m, 1H), 4.11 (dd, J = 3.6, 11.6 Hz, 1H), 4.06-3.97 (m, 1H), 3.89-3.83 (m, 1H), 3.81-3.72 (m, 1H), 3.30-3.24 (m, 1H), 2.96-2.81 (m, 1H), 2.79-2.62 (m, 2H), 2.40 (t, J = 11.2 Hz, 1H), 2.18 (s, 3H), 2.16-2.01 (m, 3H), 1.98-1.84 (m, 4H), 1.77-1.65 (m, 2H). |
| 13 | 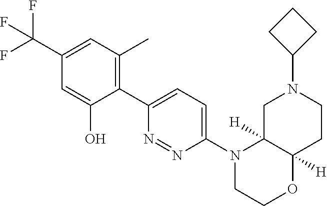 [SMILES: Cc1cc(C(F)(F)F)cc(O)c1-c1ccc(N2CCO[C@@H]3CCN(C4CCC4)C[C@@H]32)nn1] | 449 0.516 D | 1H NMR (400 MHz, CD3OD) δ ppm 7.47 (d, J = 9.6 Hz, 1H), 7.28 (d, J = 9.6 Hz, 1H), 7.09 (s, 1H), 7.02 (s, 1H), 4.55-4.45 (m, 1H), 4.11 (dd, J = 3.6, 11.6 Hz, 1H), 4.06-3.97 (m, 1H), 3.89-3.83 (m, 1H), 3.81-3.72 (m, 1H), 3.30-3.24 (m, 1H), 2.96-2.81 (m, 1H), 2.79-2.62 (m, 2H), 2.40 (t, J = 11.2 Hz, 1H), 2.18 (s, 3H), 2.16-2.01 (m, 3H), 1.98-1.84 (m, 4H), 1.77-1.65 (m, 2H). |
| 14 | 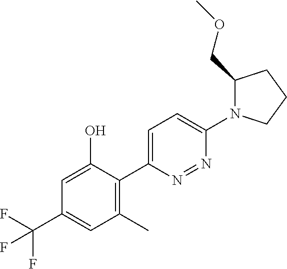 [SMILES: COC[C@H]1CCCN1c1ccc(-c2c(C)cc(C(F)(F)F)cc2O)nn1] | 368.2 1.19 B | 1H NMR (400 MHz, DMSO-d6, 30° C.) δ ppm 1.91- 2.11 (m, 4 H) 2.14 (s, 3 H) 3.29 (s, 3 H) 3.32-3.42 (m, 2 H) 3.50-3.62 (m, 2 H) 4.24-4.36 (m, 1 H) 6.98 (d, J = 9.4 Hz, 1 H) 7.06 (br s, 1 H) 7.11 (br s, 1 H) 7.34 (d, J = 9.4 Hz, 1 H) 10.07-10.13 (m, 1 H) |
| 15 | 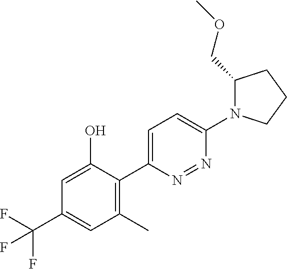 [SMILES: COC[C@@H]1CCCN1c1ccc(-c2c(C)cc(C(F)(F)F)cc2O)nn1] | 368.2 1.20 B | 1H NMR (400 MHz, DMSO-d6, 30° C.) δ ppm 1.90- 2.10 (m, 4 H) 2.14 (s, 3 H) 3.29 (s, 3 H) 3.32-3.43 (m, 2 H) 3.50-3.62 (m, 2 H) 4.25-4.36 (m, 1 H) 6.98 (d, J = 9.4 Hz, 1 H) 7.06 (br s, 1 H) 7.11 (br s, 1 H) 7.34 (d, J = 9.4 Hz, 1 H) 10.11 (s, 1 H) |
| 16 | 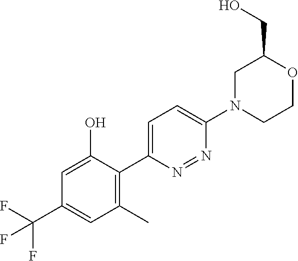 [SMILES: Cc1cc(C(F)(F)F)cc(O)c1-c1ccc(N2CCO[C@H](CO)C2)nn1] | 370.2 1.21 B | 1H NMR (400 MHz, DMSO-d6, 30° C.) δ ppm 2.13 (s, 3 H), 2.74 (dd, J = 12.8, 9.9 Hz, 1 H), 2.95-3.08 (m, 1 H), 3.41-3.58 (m, 3 H), 3.62 (td, J = 11.6, 2.8 Hz, 1 H), 3.97-4.05 (m, 1 H), 4.15 (br d, J = 13.0 Hz, 1 H), 4.34 (br d, J = 12.6 Hz, 1 H), 4.80 (t, J = 5.6 Hz, 1 H), 7.06 (br s, 1 H), 7.12 (br s, 1 H), 7.31 (d, J = 9.5 Hz, 1 H), 7.41 (d, J = 9.5 Hz, 1 H), 10.45 (s, 1 H) |
| 17 | 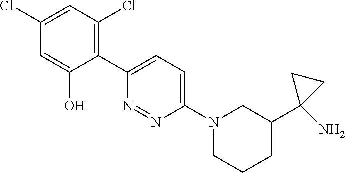 [SMILES: NC1(C2CCCN(c3ccc(-c4c(O)cc(Cl)cc4Cl)nn3)C2)CC1] | 379.2 0.64 A | 1H NMR (400 MHz, DMSO-d6, 30° C.) δ ppm 0.42- 0.66 (m, 4 H) 1.12-1.29 (m, 1 H) 1.39-1.57 (m, 2 H) 1.69-1.86 (m, 2 H) 2.73-2.90 (m, 2 H) 4.40 (br d, J = 12.8 Hz, 1 H) 4.48 (br d, J = 12.5 Hz, 1 H) 6.96 (d, J = 2.0 Hz, 1 H) 7.13 (d, J = 2.0 Hz, 1 H) 7.29 (d, J = 9.5 Hz, 1 H) 7.33 (d, J = 9.5 Hz, 1 H) |
| 18 | 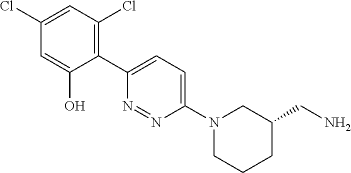 [SMILES: NC[C@@H]1CCCN(c2ccc(-c3c(O)cc(Cl)cc3Cl)nn2)C1] | 353.2 0.59 A | 1H NMR (400 MHz, DMSO-d6, 30° C.) δ ppm 1.17- 1.34 (m, 1 H), 1.41-1.66 (m, 2 H), 1.68-1.79 (m, 1 H), 1.81-1.91 (m, 1 H), 2.56 (br d, J = 6.1 Hz, 2 H), 2.73 (dd, J = 12.9, 10.4 Hz, 1 H), 2.94-3.04 (m, 1 H), 4.21-4.32 (m, 1 H), 4.33-4.43 (m, 1 H), 6.88 (br s, 1 H), 7.02 (br s, 1 H), 7.27 (d, J = 9.5 Hz, 1 H), 7.32 (d, J = 9.5 Hz, 1 H) |
| 19 | 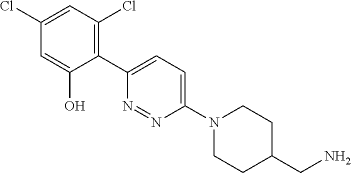 [SMILES: NCC1CCN(c2ccc(-c3c(O)cc(Cl)cc3Cl)nn2)CC1] | 353.2 0.53 A | 1H NMR (400 MHz, DMSO-d6, 30° C.) δ ppm 1.06- 1.22 (m, 2 H) 1.51-1.66 (m, 1 H) 1.81 (br d, J = 11.0 Hz, 2 H) 2.37-2.58 (m hidden, 2 H) 2.89 (td, J = 12.6, 2.2 Hz, 2 H) 4.42 (br d, J = 13.3 Hz, 2 H) 6.83 (d, J = 2.0 Hz, 1 H) 6.96 (d, J = 2.0 Hz, 1 H) 7.27 (d, J = 9.5 Hz, 1 H) 7.30 (d, J = 9.5 Hz, 1 H) |
| 20 | 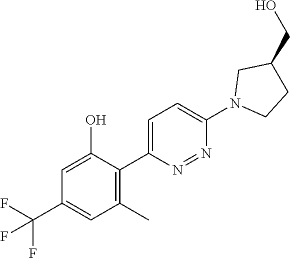 [SMILES: Cc1cc(C(F)(F)F)cc(O)c1-c1ccc(N2CC[C@H](CO)C2)nn1] | 354.3 1.00 B | 1H NMR (400 MHz, DMSO-d6, 30° C.) δ ppm 1.80 (dq, J = 12.4, 7.6 Hz, 1 H), 2.07 (dtd, J = 12.3, 7.2, 7.2, 5.3 Hz, 1 H), 2.14 (s, 3 H), 2.40-2.54 (m partially hidden, 1 H), 3.22-3.34 (m partially hidden, 1 H), 3.38-3.53 (m, 3 H), 3.55-3.67 (m, 2 H), 4.73 (t, J = 5.3 Hz, 1 H), 6.90 (d, J = 9.5 Hz, 1 H), 7.06 (br s, 1 H), 7.11 (br s, 1 H), 7.33 (d, J = 9.5 Hz, 1 H), 10.10 (s, 1 H) |
| 21 | 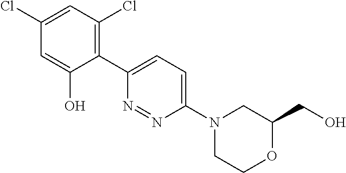 [SMILES: OC[C@@H]1CN(c2ccc(-c3c(O)cc(Cl)cc3Cl)nn2)CCO1] | 356.2 1.28 B | 1H NMR (400 MHz, DMSO-d6, 30° C.) δ ppm 2.74 (dd, J = 12.8, 9.8 Hz, 1 H), 2.95-3.06 (m, 1 H), 3.40- 3.57 (m, 3 H), 3.61 (td, J = 11.6, 2.8 Hz, 1 H), 3.95- 4.07 (m, 1 H), 4.15 (br d, J = 13.1 Hz, 1 H), 4.34 (br d, J = 12.8 Hz, 1 H), 4.75-4.91 (m, 1 H), 6.95 (d, J = 2.0 Hz, 1 H), 7.12 (d, J = 2.0 Hz, 1 H), 7.30 (d, J = 9.5 Hz, 1 H), 7.39 (d, J = 9.5 Hz, 1 H), 9.89-11.08 (m, 1 H) |
| 22 | 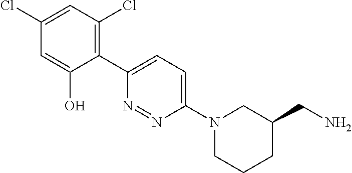 [SMILES: NC[C@H]1CCCN(c2ccc(-c3c(O)cc(Cl)cc3Cl)nn2)C1] | 353.2 0.94 B | 1H NMR (400 MHz, DMSO-d6, 30° C.) δ ppm 1.15- 1.31 (m, 1 H), 1.40-1.62 (m, 2 H), 1.66-1.79 (m, 1 H), 1.81-1.92 (m, 1 H), 2.32-2.62 (m hidden, 2 H), 2.72 (dd, J = 12.7, 10.4 Hz, 1 H), 2.93-3.03 (m, 1 H), 4.27 (br d, J = 13.0 Hz, 1 H), 4.32 4.39 (m, 1 H), 6.84 (br s, 1 H), 6.96 (br s, 1 H), 7.26 (d, J = 9.5 Hz, 1 H), 7.31 (d, J = 9.5 Hz, 1 H) |
| 23 | 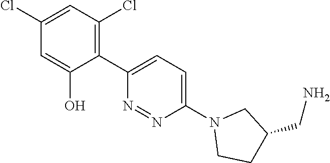 [SMILES: NC[C@@H]1CCN(c2ccc(-c3c(O)cc(Cl)cc3Cl)nn2)C1] | 339.2 0.78 B | 1H NMR (400 MHz, DMSO-d6, 30° C.) δ ppm 1.72- 1.86 (m, 1 H) 2.05-2.21 (m, 1 H) 2.36-2.48 (m, 2 H) 2.77 (br d, J = 6.8 Hz, 2 H) 3.26 (dd, J = 10.7, 6.8 Hz, 1 H) 3.42-3.53 (m, 1 H) 3.54-3.63 (m, 1 H) 3.68 (br dd, J = 10.6, 7.6 Hz, 1 H) 6.87 (d, J = 9.5 Hz, 1 H) 6.92 (d, J = 2.0 Hz, 1 H) 7.08 (d, J = 2.0 Hz, 1 H) 7.32 (d, J = 9.5 Hz, 1 H) |
| 24 | 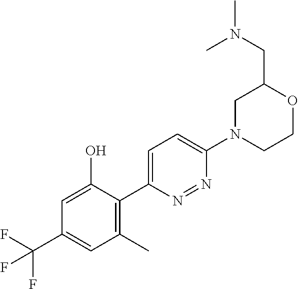 [SMILES: Cc1cc(C(F)(F)F)cc(O)c1-c1ccc(N2CCOC(CN(C)C)C2)nn1] | 397.3 0.96 B | 1H NMR (400 MHz, DMSO-d6, 30° C.) δ ppm 2.13 (s, 3 H) 2.21 (s, 6 H) 2.33-2.46 (m, 2 H) 2.70 (dd, J = 12.9, 10.4 Hz, 1 H) 2.99 (td, J = 12.3, 3.5 Hz, 1 H) 3.55-3.72 (m, 2 H) 3.98 (dd, J = 11.6, 2.2 Hz, 1 H) 4.13 (br d, J = 12.6 Hz, 1 H) 4.34 (br d, J = 12.4 Hz, 1 H) 7.06 (br s, 1 H) 7.12 (br s, 1 H) 7.31 (d, J = 9.5 Hz, 1 H) 7.40 (d, J = 9.5 Hz, 1 H) 10.06-10.22 (m, 1 H) |
| 25 | 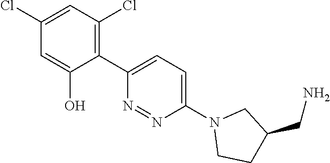 [SMILES: NC[C@H]1CCN(c2ccc(-c3c(O)cc(Cl)cc3Cl)nn2)C1] | 339.2 0.78 B | 1H NMR (400 MHz, DMSO-d6, 30° C.) δ ppm 1.67- 1.83 (m, 1 H), 2.01-2.17 (m, 1 H), 2.28-2.42 (m, 1 H), 2.61-2.75 (m, 2 H), 3.23 (dd, J = 10.6, 6.8 Hz, 2 H), 3.33-3.82 (m partially hidden, 3 H), 6.82-6.87 (m, 2 H), 6.98 (d, J = 2 Hz, 1 H), 7.29 (d, J = 9.5 Hz, 1 H) |
| 26 | 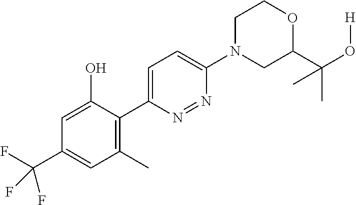 [SMILES: Cc1cc(C(F)(F)F)cc(O)c1-c1ccc(N2CCOC(C(C)(C)O)C2)nn1] | 398.3 1.35 B | 1H NMR (400 MHz, DMSO-d6, 30° C.) δ ppm 1.14 (s, 3 H), 1.18 (s, 3 H), 2.13 (s, 3 H), 2.80 (dd, J = 12.7, 11.1 Hz, 1 H), 2.95 (td, J = 12.4, 3.4 Hz, 1 H), 3.20-3.35 (m partially hidden, 1 H), 3.60 (td, J = 11.7, 2.5 Hz, 1 H), 4.04 (dd, J = 11.4, 2.9 Hz, 1 H), 4.16 (br d, J = 12.6 Hz, 1 H), 4.44 (br d, J = 13.0 Hz, 1 H), 4.53 (s, 1 H), 7.06 (br s, 1 H), 7.12 (br s, 1 H), 7.30 (d, J = 9.5 Hz, 1 H), 7.40 (d, J = 9.5 Hz, 1 H), 10.15 (br s, 1 H) |
| 27 | 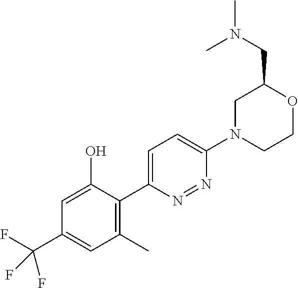 [SMILES: Cc1cc(C(F)(F)F)cc(O)c1-c1ccc(N2CCO[C@H](CN(C)C)C2)nn1] | 397.3 0.96 B | 1H NMR (400 MHz, DMSO-d6, 30° C.) δ ppm 2.13 (s, 3 H) 2.24 (s, 6 H) 2.35-2.50 (m, 2 H) 2.70 (dd, J = 12.9, 10.4 Hz, 1 H) 3.00 (td, J = 12.3, 3.4 Hz, 1 H) 3.55-3.72 (m, 2 H) 3.98 (dd, J = 11.6, 2.2 Hz, 1 H) 4.13 (br d, J = 13.0 Hz, 1 H) 4.34 (br d, J = 13.0 Hz, 1 H) 7.07 (br s, 1 H) 7.12 (br s, 1 H) 7.32 (d, J = 9.5 Hz, 1 H) 7.41 (d, J = 9.5 Hz, 1 H) 10.14 (br s, 1 H) |
| 28 | 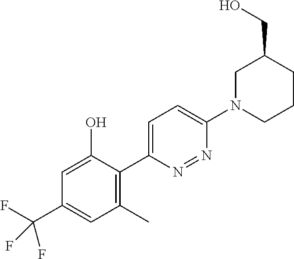 [SMILES: Cc1cc(C(F)(F)F)cc(O)c1-c1ccc(N2CCC[C@H](CO)C2)nn1] | 368.3 1.15 B | 1H NMR (400 MHz, DMSO-d6, 30° C.) δ ppm 1.19- 1.33 (m, 1 H), 1.43-1.58 (m, 1 H), 1.61-1.85 (m, 3 H), 2.14 (s, 3 H), 2.74 (dd, J = 13.0, 10.4 Hz, 1 H), 2.93-3.08 (m, 1 H), 3.23-3.49 (m partially hidden, 2 H), 4.25 (br d, J = 13.0 Hz, 1 H), 4.34-4.46 (m, 1 H), 4.56 (br t, J = 4.8 Hz, 1 H), 7.06 (br s, 1 H), 7.11 (br s, 1 H), 7.26 (d, J = 9.5 Hz, 1 H), 7.34 (d, J = 9.5 Hz, 1 H), 10.14 (br s, 1 H) |
| 29 | 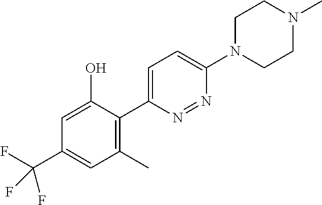 [SMILES: Cc1cc(C(F)(F)F)cc(O)c1-c1ccc(N2CCN(C)CC2)nn1] | 353.3 0.92 B | 1H NMR (400 MHz, DMSO-d6, 30° C.) δ ppm 2.13 (s, 3 H) 2.25 (s, 3 H) 2.38-2.47 (m, 4 H) 3.56-3.67 (m, 4 H) 7.06 (br s, 1 H) 7.11 (br s, 1 H) 7.31 (d, J = 9.5 Hz, 1 H) 7.37 (d, J = 9.5 Hz, 1 H) 10.03-10.20 (m, 1 H) |
| 30 | 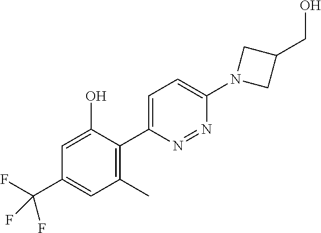 [SMILES: Cc1cc(C(F)(F)F)cc(O)c1-c1ccc(N2CC(CO)C2)nn1] | 340.2 0.99 B | 1H NMR (400 MHz, DMSO-d6, 30° C.) δ ppm 2.11 (s, 3 H) 2.80-2.95 (m, 1 H) 3.62 (br d, J = 6.1 Hz, 2 H) 3.83 (dd, J = 8.3, 5.5 Hz, 2 H) 4.10 (t, J = 8.2 Hz, 2 H) 4.68-4.97 (m, 1 H) 6.81 (d, J = 9.5 Hz, 1 H) 7.05 (br s, 1 H) 7.11 (br s, 1 H) 7.34 (d, J = 9.5 Hz, 1 H) 9.95-10.26 (m, 1 H) |
| 31 | 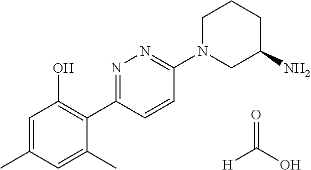 [SMILES: Cc1cc(C)c(-c2ccc(N3CCC[C@@H](N)C3)nn2)c(O)c1 ; O=CO] | 299.3 0.56 A | 1H NMR (400 MHz, DMSO-d6, 30° C.) δ ppm 1.38- 1.62 (m, 2 H) 1.73-1.88 (m, 1 H) 1.90-2.00 (m, 1 H) 2.04 (s, 3 H) 2.22 (s, 3 H) 2.93-3.04 (m, 2 H) 3.08 (ddd, J = 13.0, 10.3, 2.8 Hz, 1 H) 3.96-4.06 (m, 1 H) 4.29-4.40 (m, 1 H) 6.57 (br s, 1 H) 6.58 (br s, 1 H) 7.26 (d, J = 9.5 Hz, 1 H) 7.32 (d, J = 9.5 Hz, 1 H) 8.15-8.42 (m, 1 H) 9.10-9.83 (m, 1 H) |
| 32 | 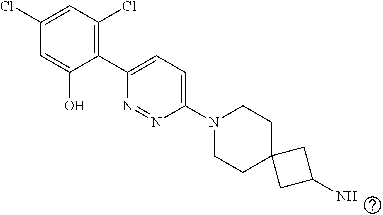 [SMILES: NC1CC2(CCN(c3ccc(-c4c(O)cc(Cl)cc4Cl)nn3)CC2)C1] | 379.2 1.05 A | 1H NMR (400 MHz, DMSO-d6, 30° C.) δ ppm 1.43- 1.66 (m, 6 H), 2.16 (ddd, J = 9.7, 7.8, 2.4 Hz, 2 H), 3.31-3.47 (m partially hidden, 1 H), 3.48-3.67 (m partially hidden, 4 H), 6.82 (d, J = 2.0 Hz, 1 H), 6.95 (d, J = 2.0 Hz, 1 H), 7.26 (d, J = 9.5 Hz, 1 H), 7.29 (d, J = 9.5 Hz, 1 H) |
| 33 | 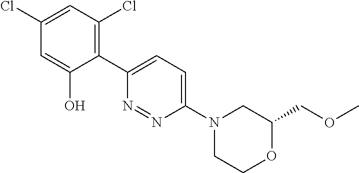 [SMILES: COC[C@H]1CN(c2ccc(-c3c(O)cc(Cl)cc3Cl)nn2)CCO1] | 354.2 0.82 A | 1H NMR (400 MHz, DMSO-d6, 30° C.) δ ppm 2.77 (dd, J = 12.8, 10.7 Hz, 1 H), 3.00 (td, J = 12.3, 3.4 Hz, 1 H), 3.31 (s, 3 H), 3.38-3.53 (m partially hidden, 2 H), 3.61 (td, J = 11.6, 2.6 Hz, 1 H), 3.69 (dtd, J = 10.4, 5.1, 5.1, 2.7 Hz, 1 H), 3.99 (br dd, J = 11.6, 2.3 Hz, 1 H), 4.15 (br d, J = 12.8 Hz, 1 H), 4.29 (br d, J = 12.5 Hz, 1 H), 6.95 (d, J = 2.0 Hz, 1 H), 7.12 (d, J = 2.0 Hz, 1 H), 7.31 (d, J = 9.5 Hz, 1 H), 7.39 (d, J = 9.5 Hz, 1 H), 9.74-11.74 (m, 1 H) |
| 34 | 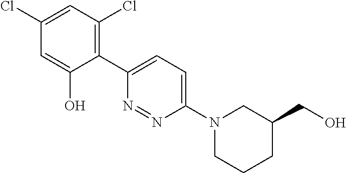 [SMILES: OC[C@H]1CCCN(c2ccc(-c3c(O)cc(Cl)cc3Cl)nn2)C1] | 354.2 0.82 A | 1H NMR (400 MHz, DMSO-d6, 30° C.) δ ppm 1.26- 1.40 (m, 1 H), 1.49-1.64 (m, 1 H), 1.66-1.93 (m, 3 H), 2.81 (dd, J = 13.0, 10.3 Hz, 1 H), 3.00-3.10 (m, 1 H), 3.26-3.61 (m hidden, 2 H), 4.32 (br d, J = 13.0 Hz, 1 H), 4.46 (br dd, J = 12.7, 3.1 Hz, 1 H), 4.61- 4.71 (m, 1 H), 7.01 (d, J = 2.0 Hz, 1 H), 7.18 (d, J = 2.0 Hz, 1 H), 7.32 (d, J = 9.5 Hz, 1 H), 7.39 (d, J = 9.5 Hz, 1 H), 10.03-11.42 (m, 1 H) |
| 35 | 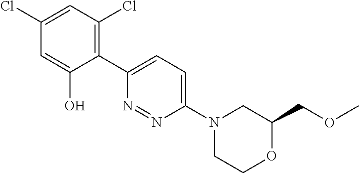 [SMILES: COC[C@@H]1CN(c2ccc(-c3c(O)cc(Cl)cc3Cl)nn2)CCO1] | 370.2 1.05 A | 1H NMR (400 MHz, DMSO-d6, 30° C.) δ ppm 2.77 (dd, J = 12.8, 10.8 Hz, 1 H) 3.00 (td, J = 12.3, 3.5 Hz, 1 H) 3.31 (s partially hidden, 3 H) 3.40-3.53 (m partially hidden, 2 H) 3.61 (td, J = 11.6, 2.6 Hz, 1 H) 3.66-3.74 (m, 1 H) 3.99 (dd, J = 11.6, 2.1 Hz, 1 H) 4.15 (br d, J = 12.8 Hz, 1 H) 4.29 (br d, J = 12.5 Hz, 1 H) 6.95 (d, J = 2.0 Hz, 1 H) 7.13 (d, J = 2.0 Hz, 1 H) 7.32 (d, J = 9.5 Hz, 1 H) 7.39 (d, J = 9.5 Hz, 1 H) 9.60-11.29 (m, 1 H) |
| 36 | 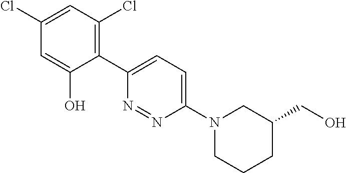 [SMILES: OC[C@@H]1CCCN(c2ccc(-c3c(O)cc(Cl)cc3Cl)nn2)C1] | 354.2 0.82 A | 1H NMR (400 MHz, DMSO-d6, 30° C.) δ ppm 1.20- 1.34 (m, 1 H), 1.43-1.57 (m, 1 H), 1.60-1.85 (m, 3 H), 2.75 (dd, J = 13.0, 10.4 Hz, 1 H), 2.96-3.05 (m, 1 H), 3.21-3.43 (m hidden, 2 H), 4.25 (br d, J = 12.9 Hz, 1 H), 4.35-4.43 (m, 1 H), 4.50-4.67 (m, 1 H), 6.95 (d, J = 2.0 Hz, 1 H), 7.11 (d, J = 2.0 Hz, 1 H), 7.25 (d, J = 9.5 Hz, 1 H), 7.32 (d, J = 9.5 Hz, 1 H), 9.55-11.35 (m, 1 H) |
| 37 | 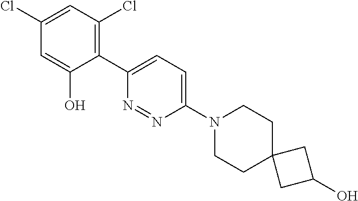 [SMILES: Oc1cc(Cl)cc(Cl)c1-c1ccc(N2CCC3(CC2)CC(O)C3)nn1] | 380.2 0.88 A | 1H NMR (400 MHz, DMSO-d6, 30° C.) δ ppm 1.52- 1.71 (m, 6 H), 2.15-2.28 (m, 2 H), 3.51-3.67 (m, 4 H), 4.07-4.24 (m, 1 H), 4.85-4.99 (m, 1 H), 6.93 (d, J = 2.0 Hz, 1 H), 7.09 (d, J = 2.0 Hz, 1 H), 7.27 (d, J = 9.5 Hz, 1 H), 7.31 (d, J = 9.5 Hz, 1 H), 10.04- 11.61 (m, 1 H) |
| 38 | 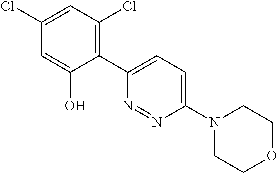 [SMILES: Oc1cc(Cl)cc(Cl)c1-c1ccc(N2CCOCC2)nn1] | 326.2 0.99 A | 1H NMR (400 MHz, DMSO-d6, 30° C.) δ ppm 3.55- 3.63 (m, 4 H), 3.72-3.86 (m, 4 H), 6.95 (d, J = 2.0 Hz, 1 H), 7.12 (d, J = 2.0 Hz, 1 H), 7.30 (d, J = 9.5 Hz, 1 H), 7.39 (d, J = 9.5 Hz, 1 H), 10.13-11.28 (m, 1 H) |
| 39 | 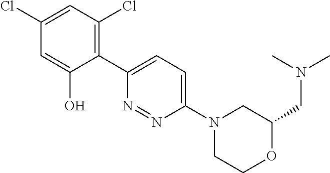 [SMILES: CN(C)C[C@H]1CN(c2ccc(-c3c(O)cc(Cl)cc3Cl)nn2)CCO1] | 383.2 0.60 A | 1H NMR (400 MHz, DMSO-d6, 30° C.) δ ppm 2.21 (s, 6 H), 2.34-2.46 (m, 2 H), 2.70 (dd, J = 13.0, 10.5 Hz, 1 H), 3.00 (td, J = 12.3, 3.5 Hz, 1 H), 3.57-3.71 (m, 2 H), 3.93-4.01 (m, 1 H), 4.13 (br d, J = 12.6 Hz, 1 H), 4.33 (br d, J = 12.4 Hz, 1 H), 6.96 (d, J = 2.0 Hz, 1 H), 7.13 (d, J = 2.0 Hz, 1 H), 7.30 (d, J = 9.5 Hz, 1 H), 7.38 (d, J = 9.5 Hz, 1 H), 9.64-11.57 (m, 1 H) |
| 40 | 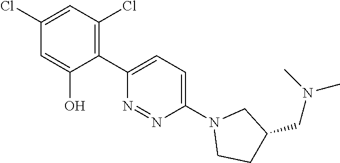 [SMILES: CN(C)C[C@@H]1CCN(c2ccc(-c3c(O)cc(Cl)cc3Cl)nn2)C1] | 367.2 0.48 A | 1H NMR (400 MHz, DMSO-d6, 30° C.) δ ppm 1.73 (dq, J = 12.2, 7.8 Hz, 1 H), 2.06-2.16 (m, 1 H), 2.18 (s, 6 H), 2.23-2.38 (m, 2 H), 2.48-2.61 (m partially hidden, 1 H), 3.20 (br dd, J = 10.5, 6.8 Hz, 1 H), 3.41- 3.53 (m, 1 H), 3.56-3.75 (m, 2 H), 6.89 (d, J = 9.3 Hz, 1 H), 6.94 (d, J = 2.0 Hz, 1 H), 7.11 (d, J = 2.0 Hz, 1 H), 7.30 (d, J = 9.3 Hz, 1 H), 9.78- 11.70 (m, 1 H) |
| 41 | 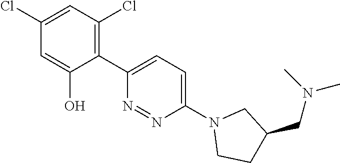 [SMILES: CN(C)C[C@H]1CCN(c2ccc(-c3c(O)cc(Cl)cc3Cl)nn2)C1] | 367.2 0.48 A | 1H NMR (400 MHz, DMSO-d6, 30° C.) δ ppm 1.73 (dq, J = 12.3, 7.8 Hz, 1 H), 2.03-2.17 (m, 1 H), 2.18 (s, 6 H), 2.23-2.36 (m, 2 H), 2.48-2.59 (m partially hidden, 1 H), 3.20 (br dd, J = 10.5, 6.8 Hz, 1 H), 3.38-3.52 (m, 1 H), 3.55-3.69 (m, 2 H), 6.89 (d, J = 9.4 Hz, 1 H), 6.95 (d, J = 2.0 Hz, 1 H), 7.11 (d, J = 2.0 Hz, 1 H), 7.30 (d, J = 9.4 Hz, 1 H), 9.49- 11.46 (m, 1 H) |
| 42 | 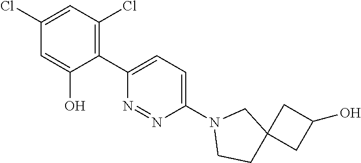 [SMILES: Oc1cc(Cl)cc(Cl)c1-c1ccc(N2CCC3(CC(O)C3)C2)nn1] | 366.2 0.70 A | 1H NMR (400 MHz, DMSO-d6, 30° C., mixture of diastereoisomer 50/50) δ ppm 1.84-2.04 (m, 4 H), 2.20-2.34 (m, 2 H), 3.37-3.59 (m, 4 H), 4.11-4.26 (m, 1 H), 4.99-5.14 (m, 1 H), 6.84-6.89 (m, 1 H), 6.94 (d, J = 2.0 Hz, 1 H), 7.10 (d, J = 2.0 Hz, 1 H), 7.26-7.33 (m, 1 H), 9.72-11.84 (m, 1 H) |
| 43 | 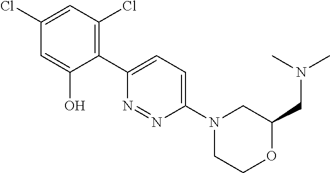 [SMILES: CN(C)C[C@@H]1CN(c2ccc(-c3c(O)cc(Cl)cc3Cl)nn2)CCO1] | 383.2 0.60 A | 1H NMR (400 MHz, DMSO-d6, 30° C.) δ ppm 2.21 (s, 6 H), 2.34-2.45 (m, 2 H), 2.70 (dd, J = 12.9, 10.4 Hz, 1 H), 3.00 (td, J = 12.3, 3.5 Hz, 1 H), 3.57-3.70 (m, 2 H), 3.98 (br dd, J = 11.6, 2.1 Hz, 1 H), 4.13 (br d, J = 12.9 Hz, 1 H), 4.33 (br d, J = 12.6 Hz, 1 H), 6.96 (d, J = 2.0 Hz, 1 H), 7.13 (d, J = 2.0 Hz, 1 H), 7.30 (d, J = 9.5 Hz, 1 H), 7.38 (d, J = 9.5 Hz, 1 H), 9.86-11.64 (m, 1 H) |
| 44 | 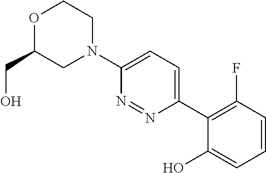 [SMILES: OC[C@@H]1CN(c2ccc(-c3c(O)cccc3F)nn2)CCO1] | 306.2 0.82 A | 1H NMR (400 MHz, DMSO-d6, 30° C.) δ ppm 2.79 (dd, J = 12.9, 9.7 Hz, 1 H), 3.03 (ddd, J = 12.8, 11.9, 3.6 Hz, 1 H), 3.42-3.69 (m, 6 H), 3.95-4.04 (m, 1 H), 4.16 (br d, J = 13.1 Hz, 1 H), 4.34 (br d, J = 12.8 Hz, 1 H), 6.75 (ddd, J = 11.4, 8.3, 0.9 Hz, 1 H), 6.80 (d, J = 8.3 Hz, 1 H), 7.26 (td, J = 8.3, 6.8 Hz, 1 H), 7.44 (d, J = 9.8 Hz, 1 H), 7.81 (d, J = 9.8 Hz, 1 H) |
| 45 | 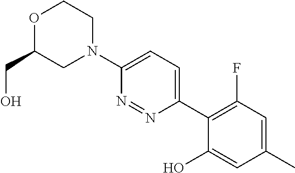 [SMILES: Cc1cc(O)c(-c2ccc(N3CCO[C@H](CO)C3)nn2)c(F)c1] | 320.2 0.95 A | 1H NMR (400 MHz, DMSO-d6, 30° C.) δ ppm 2.28 (s, 3 H), 2.78 (dd, J = 12.9, 9.8 Hz, 1 H), 3.03 (td, J = 12.3, 3.5 Hz, 1 H), 3.40-3.56 (m, 3 H), 3.60 (td, J = 11.6, 2.7 Hz, 1 H), 3.99 (dd, J = 11.5, 2.3 Hz, 1 H), 4.15 (br d, J = 13.0 Hz, 1 H), 4.33 (br d, J = 12.8 Hz, 1 H), 4.81-4.89 (m, 1 H), 6.58-6.66 (m, 2 H), 7.46 (d, J = 9.8 Hz, 1 H), 7.84 (d, J = 9.8 Hz, 1 H), 12.78 (br s, 1 H) |
| 46 | 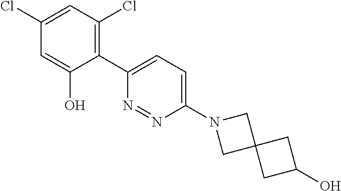 [SMILES: Oc1cc(Cl)cc(Cl)c1-c1ccc(N2CC3(CC(O)C3)C2)nn1] | 352.2 0.70 A | 1H NMR (400 MHz, DMSO-d6, 30° C.) δ ppm 1.98- 2.19 (m, 2 H), 2.42-2.62 (m hidden, 2 H), 3.95- 4.20 (m, 5 H), 5.09 (d, J = 6.2 Hz, 1 H), 6.79 (d, J = 9.2 Hz, 1 H), 6.95 (d, J = 2.0 Hz, 1 H), 7.13 (d, J = 2.0 Hz, 1 H), 7.33 (d, J = 9.2 Hz, 1 H), 10.59 (br s, 1 H) |
| 47 | 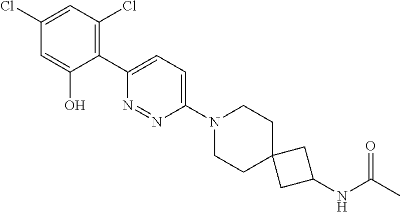 [SMILES: CC(=O)NC1CC2(CCN(c3ccc(-c4c(O)cc(Cl)cc4Cl)nn3)CC2)C1] | 421.2 0.89 A | 1H NMR (400 MHz, DMSO-d6, 30° C.) δ ppm 1.53- 1.73 (m, 6 H), 1.78 (s, 3 H), 2.16-2.26 (m, 2 H), 3.53-3.67 (m, 4 H), 4.21 (sxt, J = 8.1 Hz, 1 H), 6.95 (d, J = 2.0 Hz, 1 H), 7.12 (d, J = 2.0 Hz, 1 H), 7.26- 7.37 (m, 2 H), 8.09 (d, J = 7.5 Hz, 1 H), 10.63 (br s, 1 H) |
| 48 | 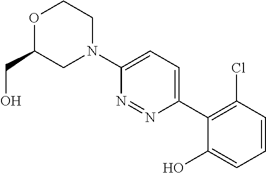 [SMILES: OC[C@@H]1CN(c2ccc(-c3c(O)cccc3Cl)nn2)CCO1] | 322.1 0.63 A | 1H NMR (400 MHz, DMSO-d6, 30° C.) δ ppm 2.74 (dd, J = 12.8, 9.8 Hz, 1 H) 2.99 (td, J = 12.3, 3.5 Hz, 1 H) 3.43-3.57 (m, 3 H) 3.61 (td, J = 11.6, 2.7 Hz, 1 H) 4.00 (dd, J = 11.6, 2.3 Hz, 1 H) 4.15 (br d, J = 13.0 Hz, 1 H) 4.34 (br d, J = 12.7 Hz, 1 H) 4.76-4.90 (m, 1 H) 6.92 (dd, J = 8.2, 0.9 Hz, 1 H) 6.99 (dd, J = 8.0, 0.9 Hz, 1 H) 7.20-7.27 (m, 1 H) 7.30 (d, J = 9.4 Hz, 1 H) 7.39 (d, J = 9.4 Hz, 1 H) 9.90-10.21 (m, 1 H) |
| 49 | 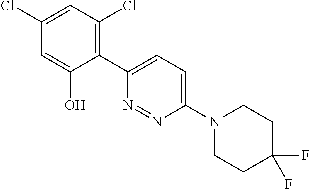 [SMILES: Oc1cc(Cl)cc(Cl)c1-c1ccc(N2CCC(F)(F)CC2)nn1] | 360.1 1.29 A | 1H NMR (400 MHz, DMSO-d6, 30° C.) δ ppm 1.99- 2.15 (m, 4 H), 3.79-3.87 (m, 4 H), 6.96 (d, J = 2.0 Hz, 1 H), 7.13 (d, J = 2.0 Hz, 1 H), 7.38-7.46 (m, 2 H), 10.24-11.20 (m, 1 H) |
| 50 | 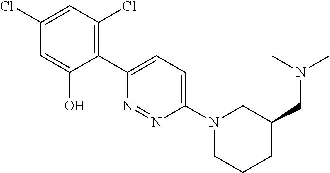 [SMILES: CN(C)C[C@H]1CCCN(c2ccc(-c3c(O)cc(Cl)cc3Cl)nn2)C1] | 381.2 0.64 A | 1H NMR (400 MHz, DMSO-d6, 30° C.) δ ppm 1.14- 1.30 (m, 1 H) 1.42-1.59 (m, 1 H) 1.68-1.88 (m, 3 H) 2.05 (dd, J = 12.0, 6.0 Hz, 1 H) 2.15 (s, 6 H) 2.17- 2.26 (m, 1 H) 2.70 (dd, J = 13.1, 9.9 Hz, 1 H) 3.01- 3.12 (m, 1 H) 4.19 (br d, J = 12.9 Hz, 1 H) 4.34-4.42 (m, 1 H) 6.95 (d, J = 2.0 Hz, 1 H) 7.12 (d, J = 2.0 Hz, 1 H) 7.24 (d, J = 9.4 Hz, 1 H) 7.32 (d, J = 9.4 Hz, 1 H) |
| 51 | 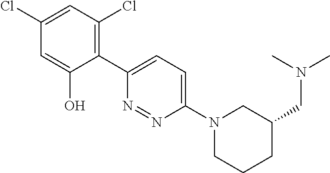 [SMILES: CN(C)C[C@@H]1CCCN(c2ccc(-c3c(O)cc(Cl)cc3Cl)nn2)C1] | 381.2 0.64 A | 1H NMR (400 MHz, DMSO-d6, 30° C.) δ ppm 1.16- 1.27 (m, 1 H), 1.44-1.57 (m, 1 H), 1.67-1.87 (m, 3 H), 2.05 (dd, J = 12.0, 6.1 Hz, 1 H), 2.14 (s, 6 H), 2.20 (dd, J = 12.0, 8.6 Hz, 1 H), 2.70 (dd, J = 13.1, 9.9 Hz, 1 H), 3.00-3.15 (m, 1 H), 4.14-4.28 (m, 1 H), 4.31- 4.46 (m, 1 H), 6.95 (d, J = 2.0 Hz, 1 H), 7.11 (d, J = 2.0 Hz, 1 H), 7.24 (d, J = 9.5 Hz, 1 H), 7.32 (d, J = 9.5 Hz, 1 H), 9.80-11.46 (m, 1 H) |
| 52 | 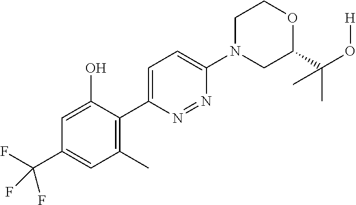 [SMILES: Cc1cc(C(F)(F)F)cc(O)c1-c1ccc(N2CCO[C@H](C(C)(C)O)C2)nn1] | 398.1 1.34 B | 1H NMR (400 MHz, DMSO-d6, 30° C.) δ ppm 1.14 (s, 3 H) 1.18 (s, 3 H) 2.12 (s, 3 H) 2.79 (dd, J = 12.8, 10.9 Hz, 1 H) 2.94 (td, J = 12.4, 3.4 Hz, 1 H) 3.21- 3.30 (m partially hidden, 1 H) 3.60 (td, J = 11.7, 2.8 Hz, 1 H) 4.04 (dd, J = 11.3, 2.8 Hz, 1 H) 4.15 (br d, J = 13.0 Hz, 1 H) 4.43 (br d, J = 12.8 Hz, 1 H) 4.46- 4.59 (m, 1 H) 6.98 (s, 2 H) 7.27 (d, J = 9.5 Hz, 1 H) 7.40 (d, J = 9.4 Hz, 1 H) |
| 53 | 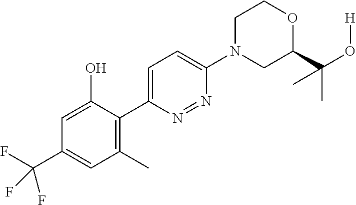 [SMILES: Cc1cc(C(F)(F)F)cc(O)c1-c1ccc(N2CCO[C@@H](C(C)(C)O)C2)nn1] | 398.1 1.34 B | 1H NMR (400 MHz, DMSO-d6, 30° C.) δ ppm 1.14 (s, 3 H), 1.18 (s, 3 H), 2.13 (s, 3 H), 2.79 (dd, J = 12.8, 10.9 Hz, 1 H), 2.94 (td, J = 12.4, 3.5 Hz, 1 H), 3.18-3.31 (m partially hidden, 1 H), 3.60 (td, J = 11.6, 2.6 Hz, 1 H), 4.04 (dd, J = 11.4, 2.8 Hz, 1 H), 4.15 (br d, J = 12.6 Hz, 1 H), 4.44 (br d, J = 12.8 Hz, 1 H), 4.52 (br s, 1 H), 7.03 (br s, 1 H), 7.06 (br s, 1 H), 7.29 (d, J = 9.5 Hz, 1 H), 7.40 (d, J = 9.5 Hz, 1 H), 9.80-11.06 (m, 1 H) |
| 54 | 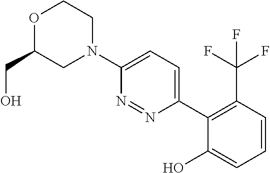 [SMILES: OC[C@@H]1CN(c2ccc(-c3c(O)cccc3C(F)(F)F)nn2)CCO1] | 356.2 0.68 A | 1H NMR (400 MHz, DMSO-d6, 30° C.) δ ppm 2.73 (dd, J = 12.9, 9.9 Hz, 1 H) 2.93-3.05 (m, 1 H) 3.42- 3.57 (m, 3 H) 3.62 (td, J = 11.6, 2.8 Hz, 1 H) 3.95- 4.04 (m, 1 H) 4.14 (br d, J = 13.4 Hz, 1 H) 4.33 (br d, J = 12.8 Hz, 1 H) 4.73-4.86 (m, 1 H) 7.19-7.30 (m, 3 H) 7.36 (d, J = 9.4 Hz, 1 H) 7.41-7.49 (m, 1 H) 10.02-10.27 (m, 1 H) |
| 55 | 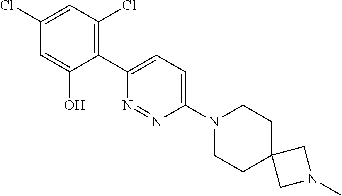 [SMILES: CN1CC2(CCN(c3ccc(-c4c(O)cc(Cl)cc4Cl)nn3)CC2)C1] | 379.2 0.52 A | 1H NMR (400 MHz, DMSO-d6, 30° C.) δ ppm 1.98- 2.23 (m, 4 H), 2.75 (br s, 3 H), 3.04-3.41 (m partially hidden, 4 H), 3.51-3.69 (m, 4 H), 6.89 (d, J = 9.3 Hz, 1 H), 6.99 (d, J = 2.0 Hz, 1 H), 7.13 (d, J = 2.0 Hz, 1 H), 7.35 (d, J = 9.3 Hz, 1 H), 10.35- 11.12 (m, 1 H) |
| 56 | 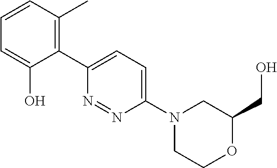 [SMILES: Cc1cccc(O)c1-c1ccc(N2CCO[C@H](CO)C2)nn1] | 302.2 0.49 A | 1H NMR (400 MHz, DMSO-d6, 30° C.) δ ppm 2.06 (s, 3 H) 2.72 (dd, J = 12.7, 9.9 Hz, 1 H) 2.98 (td, J = 12.2, 3.3 Hz, 1 H) 3.41-3.57 (m, 3 H) 3.62 (td, J = 11.5, 2.4 Hz, 1 H) 3.99 (br dd, J = 11.4, 2.0 Hz, 1 H) 4.13 (br d, J = 12.8 Hz, 1 H) 4.32 (br d, J = 12.6 Hz, 1 H) 4.80 (br s, 1 H) 6.71-6.79 (m, 2 H) 7.10 (t, J = 7.8 Hz, 1 H) 7.28 (d, J = 9.4 Hz, 1 H) 7.37 (d, J = 9.4 Hz, 1 H) 9.43 (br s, 1 H) |
| 57 | 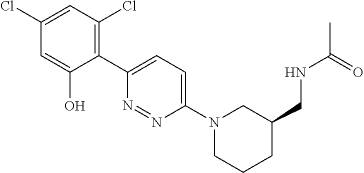 [SMILES: CC(=O)NC[C@H]1CCCN(c2ccc(-c3c(O)cc(Cl)cc3Cl)nn2)C1] | 395.2 0.83 A | 1H NMR (400 MHz, DMSO-d6, 30° C.) δ ppm 1.17- 1.30 (m, 1 H), 1.40-1.57 (m, 1 H), 1.61-1.89 (m, 3 H), 1.82 (s, 3 H), 2.66-2.77 (m, 1 H), 2.90-3.12 (m, 3 H), 4.18-4.28 (m, 1 H), 4.30-4.37 (m, 1 H), 6.94 (d, J = 2.1 Hz, 1 H), 7.10 (d, J = 2.1 Hz, 1 H), 7.27 (d, J = 9.5 Hz, 1 H), 7.33 (d, J = 9.5 Hz, 1 H), 7.90 (br t, J = 5.7 Hz, 1 H), 10.02-11.37 (m, 1 H) |
| 58 | 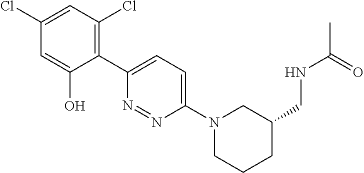 [SMILES: CC(=O)NC[C@@H]1CCCN(c2ccc(-c3c(O)cc(Cl)cc3Cl)nn2)C1] | 395.2 0.83 A | 1H NMR (400 MHz, DMSO-d6, 30° C.) δ ppm 1.15- 1.33 (m, 1 H) 1.39-1.57 (m, 1 H) 1.59-1.85 (m, 3 H) 1.84 (s, 3 H) 2.70 (dd, J = 13.1, 10.4 Hz, 1 H) 2.91-3.10 (m, 3 H) 4.22 (br d, J = 13.3 Hz, 1 H) 4.33 (br dd, J = 13.0, 3.9 Hz, 1 H) 6.95 (d, J = 2.0 Hz, 1 H) 7.12 (d, J = 2.0 Hz, 1 H) 7.27 (d, J = 9.5 Hz, 1 H) 7.34 (d, J = 9.5 Hz, 1 H) 7.90 (br t, J = 5.6 Hz, 1 H) |
| 59 | 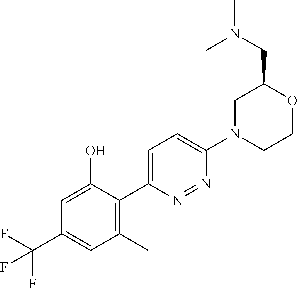 [SMILES: Cc1cc(C(F)(F)F)cc(O)c1-c1ccc(N2CCO[C@H](CN(C)C)C2)nn1] | 397.2 0.95 B | 1H NMR (400 MHz, DMSO-d6, 30° C.) δ ppm 2.13 (s, 3 H), 2.21 (s, 6 H), 2.34-2.45 (m, 2 H), 2.70 (dd, J = 13.0, 10.4 Hz, 1 H), 2.93-3.05 (m, 1 H), 3.54- 3.74 (m, 2 H), 3.94-4.03 (m, 1 H), 4.13 (br d, J = 12.9 Hz, 1 H), 4.34 (br d, J = 12.3 Hz, 1 H), 7.06 (br s, 1 H), 7.11 (br s, 1 H), 7.31 (d, J = 9.5 Hz, 1 H), 7.40 (d, J = 9.5 Hz, 1 H), 9.90-10.45 (m, 1 H) |
| 60 | 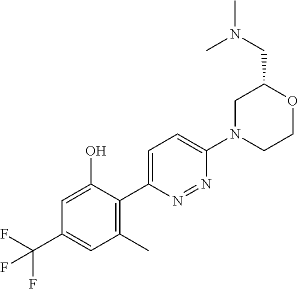 [SMILES: Cc1cc(C(F)(F)F)cc(O)c1-c1ccc(N2CCO[C@@H](CN(C)C)C2)nn1] | 397.2 0.95 B | 1H NMR (400 MHz, DMSO-d6, 30° C.) δ ppm 2.13 (s, 3 H) 2.21 (s, 6 H) 2.32-2.46 (m, 2 H) 2.70 (dd, J = 13, 10 Hz, 1 H) 2.99 (td, J = 12, 4 Hz, 1 H) 3.52- 3.73 (m, 2 H) 3.92-4.04 (m, 1 H) 4.13 (br d, J = 13 Hz, 1 H) 4.34 (br d, J = 12 Hz, 1 H) 7.06 (br s, 1 H) 7.11 (br s, 1 H) 7.31 (d, J = 9.4 Hz, 1 H) 7.40 (d, J = 9.4 Hz, 1 H) 10.02-10.30 (m, 1 H) |
| 61 | 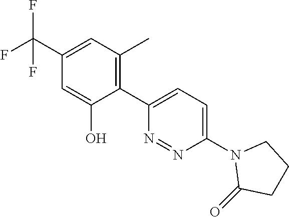 [SMILES: Cc1cc(C(F)(F)F)cc(O)c1-c1ccc(N2CCCC2=O)nn1] | 338.1 1.50 B | 1H NMR (400 MHz, DMSO-d6, 30° C.) δ ppm 2.07- 2.23 (m, 2 H), 2.11 (s, 3 H), 2.64 (t, J = 8.0 Hz, 2 H), 4.16 (t, J = 7.1 Hz, 2 H), 7.09 (br s, 1 H), 7.16 (br s, 1 H), 7.71 (d, J = 9.3 Hz, 1 H), 8.61 (d, J = 9.3 Hz, 1 H), 10.09-10.45 (m, 1 H) |
| 62 | 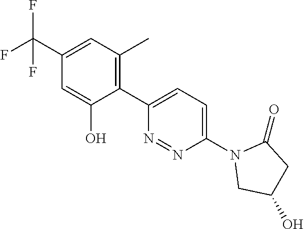 [SMILES: Cc1cc(C(F)(F)F)cc(O)c1-c1ccc(N2C[C@@H](O)CC2=O)nn1] | 354.1 1.33 B | 1H NMR (400 MHz, DMSO-d6, 30° C.) δ ppm 2.11 (s, 3 H), 2.42 (br d, J = 17.5 Hz, 1 H), 2.99 (dd, J = 17.2, 5.9 Hz, 1 H), 4.11 (br d, J = 11.8 Hz, 1 H), 4.22 (dd, J = 11.8, 4.8 Hz, 1 H), 4.44-4.53 (m, 1 H), 5.31- 5.49 (m, 1 H), 7.09 (br s, 1 H), 7.15 (br s, 1 H), 7.72 (d, J = 9.3 Hz, 1 H), 8.63 (d, J = 9.3 Hz, 1 H), 9.94- 10.64 (m, 1 H) |
| 63 | 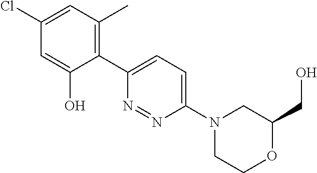 [SMILES: Cc1cc(Cl)cc(O)c1-c1ccc(N2CCO[C@H](CO)C2)nn1] | 336.2 0.67 A | 1H NMR (400 MHz, DMSO-d6, 30° C.) δ ppm 2.05 (s, 3 H), 2.72 (dd, J = 12.8, 9.8 Hz, 1 H), 2.98 (td, J = 12.3, 3.4 Hz, 1 H), 3.42-3.57 (m, 3 H), 3.61 (td, J = 11.5, 2.6 Hz, 1 H), 3.99 (dd, J = 11.5, 2.3 Hz, 1 H), 4.13 (br d, J = 12.9 Hz, 1 H), 4.32 (br d, J = 12.8 Hz, 1 H), 4.74-4.87 (m, 1 H), 6.81 (br s, 1 H), 6.84 (br s, 1 H), 7.28 (d, J = 9.4 Hz, 1 H), 7.36 (d, J = 9.4 Hz, 1H), 9.70-10.28 (m, 1 H) |
| 64 | 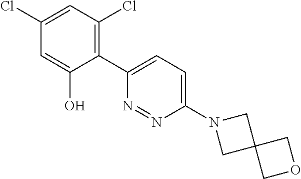 [SMILES: Oc1cc(Cl)cc(Cl)c1-c1ccc(N2CC3(COC3)C2)nn1] | 338.1 0.74 A | 1H NMR (400 MHz, DMSO-d6, 30° C.) δ ppm 4.27 (s, 4 H), 4.76 (s, 4 H), 6.84 (d, J = 9.1 Hz, 1 H), 6.94 (d, J = 1.9 Hz, 1 H), 7.10 (br s, 1 H), 7.34 (d, J = 9.1 Hz, 1 H), 10.01-11.43 (m, 1 H) |
| 65 | 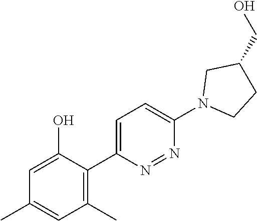 [SMILES: Cc1cc(C)c(-c2ccc(N3CC[C@@H](CO)C3)nn2)c(O)c1] | 300.2 0.83 B | 1H NMR (500 MHz, DMSO-d6, 27° C.) δ ppm 1.70- 1.85 (m, 1 H), 1.99-2.13 (m, 1 H), 2.04 (s, 3 H), 2.22 (s, 3 H), 2.40-2.52 (m partially hidden, 1 H), 3.27 (dd, J = 10.2, 6.5 Hz, 1 H), 3.38-3.52 (m, 3 H), 3.53-3.66 (m, 2 H), 4.73 (t, J = 5.4 Hz, 1 H), 6.56 (br s, 1 H), 6.58 (br s, 1 H), 6.86 (d, J = 9.5 Hz, 1 H), 7.27 (d, J = 9.5 Hz, 1 H), 9.40 (s, 1 H) |
| 66 | 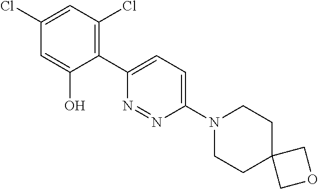 [SMILES: Oc1cc(Cl)cc(Cl)c1-c1ccc(N2CCC3(CC2)COC3)nn1] | 366.2 0.96 A | 1H NMR (400 MHz, DMSO-d6, 30° C.) δ ppm 1.84- 1.91 (m, 4 H), 3.55-3.64 (m, 4 H), 4.38 (s, 4 H), 6.95 (s, 1 H), 7.12 (s, 1 H), 7.27-7.41 (m, 2 H), 10.20-11.04 (m, 1 H) |
| 67 | 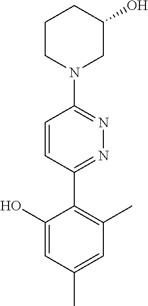 [SMILES: Cc1cc(C)c(-c2ccc(N3CCC[C@H](O)C3)nn2)c(O)c1] | 300.2 0.55 A | 1H NMR (400 MHz, DMSO-d6, 30° C.) δ ppm 1.36- 1.58 (m, 2 H), 1.72-1.83 (m, 1 H), 1.87-1.98 (m, 1 H), 2.04 (s, 3 H), 2.22 (s, 3 H), 2.90 (dd, J = 12.6, 8.8 Hz, 1 H), 3.07-3.18 (m, 1 H), 3.52-3.64 (m, 1 H), 3.94-4.03 (m, 1 H), 4.21 (dd, J = 12.4, 3.7 Hz, 1 H), 4.87 (d, J = 4.4 Hz, 1 H), 6.56 (br s, 1 H), 6.58 (br s, 1 H), 7.22 (d, J = 9.5 Hz, 1 H), 7.27 (d, J = 9.5 Hz, 1 H), 9.42 (s, 1 H) |
| 68 | 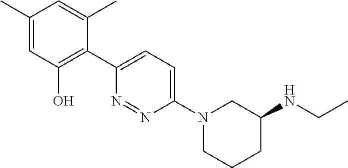 [SMILES: CCN[C@H]1CCCN(c2ccc(-c3c(C)cc(C)cc3O)nn2)C1] | 327.3 0.45 A | 1H NMR (400 MHz, DMSO-d6, 30° C.) δ ppm 1.02 (t, J = 7 Hz, 3 H), 1.25-1.36 (m, 1 H), 1.42-1.56 (m, 1 H), 1.54-1.72 (m, 1 H), 1.71-1.82 (m, 1 H), 1.91- 2.00 (m, 1 H), 2.04 (s, 3 H), 2.22 (s, 3 H), 2.50- 2.71 (m, 3 H), 2.76 (dd, J = 13, 9 Hz, 1 H), 2.95-3.07 (m, 1 H), 4.10-4.17 (m, 1 H), 4.30-4.38 (m, 1 H), 6.56 (br s, 1 H), 6.58 (br s, 1 H), 7.22 (d, J = 9.5 Hz, 1 H), 7.28 (d, J = 9.5 Hz, 1 H), 9.33-9.52 (m, 1 H) |
| 69 | 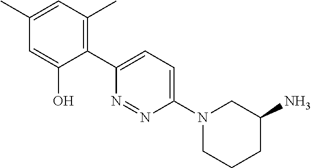 [SMILES: Cc1cc(C)c(-c2ccc(N3CCC[C@H](N)C3)nn2)c(O)c1] | 299.2 0.42 A | 1H NMR (400 MHz, DMSO-d6, 30° C.) δ ppm 1.20- 1.35 (m, 1 H), 1.41-1.58 (m, 1 H), 1.65-2.34 (m, 2 H), 1.69-1.80 (m, 1 H), 1.85-1.95 (m, 1 H), 2.04 (s, 3 H), 2.22 (s, 3 H), 2.62-2.78 (m, 2 H), 2.94 (ddd, J = 13, 11, 3 Hz, 1 H), 4.11-4.22 (m, 1 H), 4.27 (br dd, J = 10, 1 Hz, 1 H), 6.56 (br s, 1 H), 6.58 (br s, 1 H), 7.22 (d, J = 9.5 Hz, 1 H), 7.28 (d, J = 9.5 Hz, 1 H), 9.07-9.87 (m, 1 H) |
| 70 | 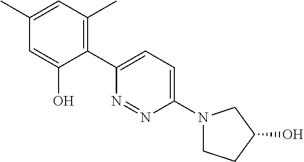 [SMILES: Cc1cc(C)c(-c2ccc(N3CC[C@@H](O)C3)nn2)c(O)c1] | 286.3 1.03 E | 1H NMR (500 MHz, DMSO) δ (ppm): 9.51 (s, 1H), 7.27 (d, J = 9.3 Hz, 1H), 6.86 (d, J = 9.3 Hz, 1H), 6.55 (d, J = 13.7 Hz, 2H), 5.03 (s, 1H), 4.44 (s, 1H), 3.60-3.50 (m, 3H), 3.41 (d, J = 11.2 Hz, 1H), 2.21 (s, 3H), 2.07 (m, 1H), 2.03 (s, 3H), 1.98-1.91 (m, 1H). |
| 71 | 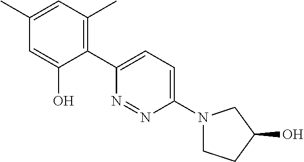 [SMILES: Cc1cc(C)c(-c2ccc(N3CC[C@H](O)C3)nn2)c(O)c1] | 286.3 1.04 E | 1H NMR (400 MHz, DMSO) δ (ppm): 9.41 (s, 1H), 7.27 (d, J = 9.2 Hz, 1H), 6.87 (d, J = 9.3 Hz, 1H), 6.56 (d, J = 4.2 Hz, 2H), 5.02 (d, J = 3.6 Hz, 1H), 4.44 (s, 1H), 3.56 (td, J = 12.7, 7.3 Hz, 3H), 3.41 (d, J = 11.1 Hz, 1H), 2.22 (s, 3H), 2.08 (m, J = 9.0, 4.5 Hz, 1H), 2.03 (s, 3H), 2.00-1.89 (m, 1H) |
| 72 | 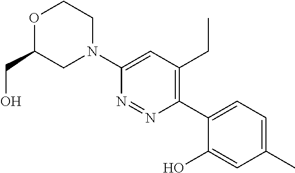 [SMILES: CCc1cc(N2CCO[C@H](CO)C2)nnc1-c1ccc(C)cc1O] | 330.1 0.95 B | 1H NMR (400 MHz, DMSO-d6, 30° C.) δ ppm 0.99 (t, J = 7.5 Hz, 3 H), 2.28 (s, 3 H), 2.42 (q, J = 7.5 Hz, 2 H), 2.69 (dd, J = 12.7, 9.7 Hz, 1 H), 2.94 (td, J = 12.3, 3.5 Hz, 1 H), 3.42-3.57 (m, 3 H), 3.61 (td, J = 11.6, 2.8 Hz, 1 H), 3.95-4.02 (m, 1 H), 4.14 (br d, J = 13.0 Hz, 1 H), 4.29 (br d, J = 12.6 Hz, 1 H), 4.79 (t, J = 5.6 Hz, 1 H), 6.70 (br d, J = 7.5 Hz, 1 H), 6.73 (br s, 1 H), 7.02 (d, J = 7.5 Hz, 1 H), 7.07 (s, 1 H), 9.45 (s, 1 H) |
| 73 | 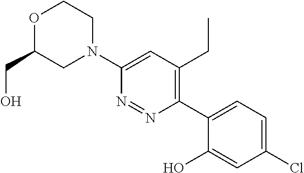 [SMILES: CCc1cc(N2CCO[C@H](CO)C2)nnc1-c1ccc(Cl)cc1O] | 350.1 1.06 B | 1H NMR (400 MHz, DMSO-d6, 30° C.) δ ppm 1.01 (t, J = 7.5 Hz, 3 H), 2.40 (q, J = 7.5 Hz, 2 H), 2.70 (dd, J = 12.8, 9.8 Hz, 1 H), 2.95 (td, J = 12.3, 3.5 Hz, 1 H), 3.42-3.57 (m, 3 H), 3.60 (td, J = 11.6, 2.6 Hz, 1 H), 3.99 (br dd, J = 11.5, 2.3 Hz, 1 H), 4.16 (br d, J = 12.9 Hz, 1 H), 4.31 (br d, J = 12.6 Hz, 1 H), 4.76-4.83 (m, 1 H), 6.93-6.98 (m, 2 H), 7.10 (br s, 1 H), 7.17 (d, J = 8.6 Hz, 1 H), 10.12 (br s, 1 H) |
| 74 | 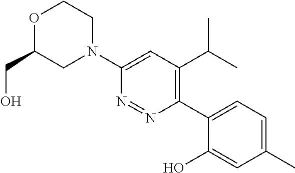 [SMILES: Cc1ccc(-c2nnc(N3CCO[C@H](CO)C3)cc2C(C)C)c(O)c1] | 344.1 1.02 B | 1H NMR (400 MHz, DMSO-d6, 30° C.) δ ppm 1.04- 1.12 (m, 6 H), 2.28 (s, 3 H), 2.60-2.80 (m, 2 H), 2.93 (td, J = 12.3, 3.5 Hz, 1 H), 3.42-3.59 (m, 3 H), 3.61 (td, J = 11.5, 2.7 Hz, 1 H), 3.95-4.05 (m, 1 H), 4.16 (br d, J = 13.0 Hz, 1 H), 4.31 (br d, J = 12.6 Hz, 1 H), 4.78 (t, J = 5.4 Hz, 1 H), 6.67-6.74 (m, 2 H), 7.01 (d, J = 7.5 Hz, 1 H), 7.09 (s, 1 H), 9.37 (s, 1 H) |
| 75 | 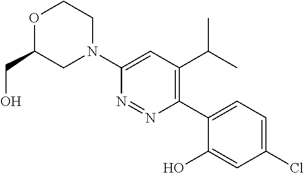 [SMILES: CC(C)c1cc(N2CCO[C@H](CO)C2)nnc1-c1ccc(Cl)cc1O] | 364.1 1.14 B | 1H NMR (400 MHz, DMSO-d6, 30° C.) δ ppm 1.07- 1.12 (m, 6 H), 2.60-2.74 (m, 2 H), 2.94 (td, J = 12.3, 3.5 Hz, 1 H), 3.42-3.57 (m, 3 H), 3.61 (td, J = 11.6, 2.7 Hz, 1 H), 3.96-4.03 (m, 1 H), 4.18 (br d, J = 12.9 Hz, 1 H), 4.32 (br d, J = 12.6 Hz, 1 H), 4.79 (t, J = 5.5 Hz, 1 H), 6.93-6.98 (m, 2 H), 7.11 (s, 1 H), 7.17 (d, J = 8.6 Hz, 1 H), 10.07 (br s, 1 H) |
| 76 | 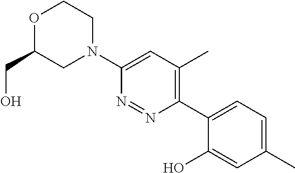 [SMILES: Cc1ccc(-c2nnc(N3CCO[C@H](CO)C3)cc2C)c(O)c1] | 316.1 0.88 B | 1H NMR (400 MHz, DMSO-d6, 30° C.) δ ppm 2.11 (s, 3 H), 2.28 (s, 3 H), 2.69 (br dd, J = 12.7, 9.7 Hz, 1 H), 2.94 (td, J = 12.3, 3.4 Hz, 1 H), 3.41-3.56 (m, 3 H), 3.60 (td, J = 11.6, 2.6 Hz, 1 H), 3.98 (br dd, J = 11.4, 2.1 Hz, 1 H), 4.12 (br d, J = 12.8 Hz, 1 H), 4.28 (br d, J = 12.6 Hz, 1 H), 4.82 (br t, J = 5.1 Hz, 1 H), 6.71 (d, J = 7.8 Hz, 1 H), 6.74 (s, 1 H), 7.06 (d, J = 7.6 Hz, 1 H), 7.13 (s, 1 H), 9.67 (br s, 1 H) |
| 77 | 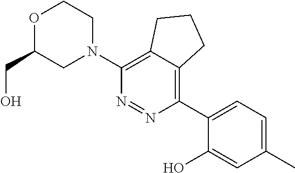 [SMILES: Cc1ccc(-c2nnc(N3CCO[C@H](CO)C3)c3c2CCC3)c(O)c1] | 342.1 1.13 B | 1H NMR (400 MHz, DMSO-d6, 30° C.) δ ppm 2.02 (quin, J = 7.1 Hz, 2 H), 2.29 (s, 3 H), 2.79 (dd, J = 12.9, 10.3 Hz, 1 H), 2.91-3.08 (m, 5 H), 3.39-3.47 (m, 1 H), 3.48-3.55 (m, 1 H), 3.59 (ddd, J = 10.1, 5.4, 2.1 Hz, 1 H), 3.67 (td, J = 11.4, 2.4 Hz, 1 H), 3.79 (br d, J = 12.6 Hz, 1 H), 3.94 (br dd, J = 12.7, 1.4 Hz, 2 H), 4.80 (t, J = 5.7 Hz, 1 H), 6.74 (d, J = 8.0 Hz, 1 H), 6.78 (s, 1 H), 7.41 (d, J = 8.0 Hz, 1 H), 12.23 (s, 1 H) |
| 78 | 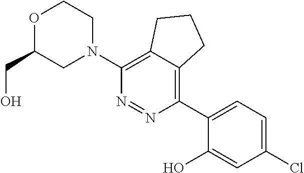 [SMILES: OC[C@@H]1CN(c2nnc(-c3ccc(Cl)cc3O)c3c2CCC3)CCO1] | 362.1 1.33 B | 1H NMR (400 MHz, DMSO-d6, 30° C.) δ ppm 2.02 (quin, J = 7.2 Hz, 2 H), 2.81 (dd, J = 12.9, 10.3 Hz, 1 H), 2.92-3.10 (m, 5 H), 3.40-3.47 (m, 1 H), 3.48- 3.55 (m, 1 H), 3.56-3.63 (m, 1 H), 3.67 (td, J = 11.4, 2.3 Hz, 1 H), 3.83 (br d, J = 12.9 Hz, 1 H), 3.91-4.02 (m, 2 H), 4.80 (br t, J = 5.5 Hz, 1 H), 6.98 (dd, J = 8.3, 2.1 Hz, 1 H), 7.01 (d, J = 2.0 Hz, 1 H), 7.50 (d, J = 8.3 Hz, 1 H), 12.28 (br s, 1 H) |
| 79 | 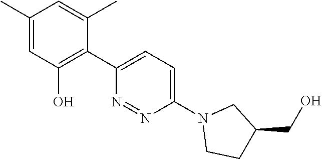 [SMILES: Cc1cc(C)c(-c2ccc(N3CC[C@H](CO)C3)nn2)c(O)c1] | 300.1 0.85 B | 1H NMR (400 MHz, DMSO-d6, 30° C.) δ ppm 1.70- 1.87 (m, 1 H) 2.04 (s, 3 H) 2.05-2.13 (m, 1 H) 2.22 (s, 3 H) 2.38-2.49 (m, 1 H) 3.17-3.31 (m partially hidden, 1 H) 3.34-3.52 (m, 3 H) 3.53-3.67 (m, 2 H) 4.71 (t, J = 5.2 Hz, 1 H) 6.56 (br s, 1 H) 6.57 (br s, 1 H) 6.85 (d, J = 9.4 Hz, 1 H) 7.27 (d, J = 9.4 Hz, 1 H) 9.39 (s, 1 H) |
| 80 | 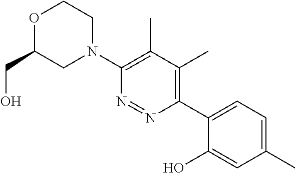 [SMILES: Cc1ccc(-c2nnc(N3CCO[C@H](CO)C3)c(C)c2C)c(O)c1] | 330.1 0.98 B | 1H NMR (400 MHz, DMSO-d6, 30° C.) δ ppm 2.03 (s, 3 H) 2.24 (s, 3 H) 2.29 (s, 3 H) 2.69-2.80 (m, 1 H) 2.88-3.03 (m, 1 H) 3.21-3.32 (m partially hidden, 1 H) 3.35-3.46 (m, 2 H) 3.46-3.57 (m, 1 H) 3.58-3.69 (m, 1 H) 3.74 (br t, J = 10.4 Hz, 1 H) 3.94 (br d, J = 11.1 Hz, 1 H) 4.75 (br s, 1 H) 6.72 (br d, J = 7.6 Hz, 1 H) 6.75 (br s, 1 H) 7.05 (d, J = 7.6 Hz, 1 H) 9.53 (br s, 1 H) |
| 81 | 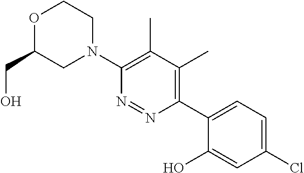 [SMILES: Cc1c(-c2ccc(Cl)cc2O)nnc(N2CCO[C@H](CO)C2)c1C] | 350.1 1.12 B | 1H NMR (400 MHz, DMSO-d6, 30° C.) δ ppm 2.03 (s, 3 H), 2.24 (s, 3 H), 2.75 (dd, J = 12.4, 10.3 Hz, 1 H), 2.96 (td, J = 11.9, 3.0 Hz, 1 H), 3.27-3.47 (m partially hidden, 3 H), 3.48-3.55 (m, 1 H), 3.61- 3.70 (m, 1 H), 3.74 (td, J = 11.3, 2.1 Hz, 1 H), 3.94 (br d, J = 10.8 Hz, 1 H), 4.76 (br t, J = 4.9 Hz, 1 H), 6.92- 7.00 (m, 2 H), 7.20 (d, J = 8.8 Hz, 1 H), 10.25 (br s, 1 H) |
| 82 | 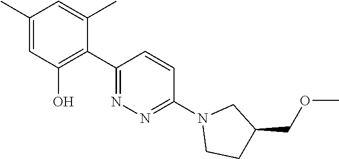 | 314 5.97 F | 1H NMR (400 MHz, CDCl3) δ ppm 11.22 (s, 1H), 7.47 (d, J = 9.6 Hz, 1H), 6.80-6.73 (m, 2H), 6.65- 6.60 (m, 1H), 3.72 (m, 2H), 3.61 (m, 1H), 3.51-3.41 (m, 2H), 3.39 (s, 3H), 3.38-3.34 (m, 1H), 2.70 (m, 1H), 2.38 (s, 3H), 2.30 (s, 3H), 2.21 (m, 1H), 1.97- 1.84 (m, 1H) |
| 83 | 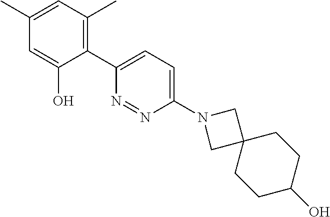 [SMILES: Cc1cc(C)c(-c2ccc(N3CC4(CCC(O)CC4)C3)nn2)c(O)c1] | 340.1 0.96 B | 1H NMR (400 MHz, DMSO-d6, 30° C.) δ ppm 1.22- 1.36 (m, 2 H), 1.47-1.61 (m, 2 H), 1.66-1.77 (m, 2 H), 1.82-1.96 (m, 2 H), 2.02 (s, 3 H), 2.22 (s, 3 H), 3.41-3.57 (m, 1 H), 3.73 (s, 2 H), 3.77 (s, 2 H), 4.49 (d, J = 4.0 Hz, 1 H), 6.52-6.60 (m, 2 H), 6.77 (d, J = 9.1 Hz, 1 H), 7.27 (d, J = 9.1 Hz, 1 H), 9.34 (s, 1 H) |
| 84 | 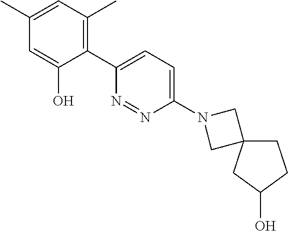 [SMILES: Cc1cc(C)c(-c2ccc(N3CC4(CCC(O)C4)C3)nn2)c(O)c1] | 326.2 0.94 B | 1H NMR (400 MHz, DMSO-d6, 30° C.) 8 ppm 1.50- 1.61 (m, 1 H), 1.76-1.90 (m, 3 H), 1.99-2.12 (m, 2 H), 2.02 (s, 3 H), 2.22 (s, 3 H), 3.92 (s, 2 H), 3.94 (d, J = 8.1 Hz, 1 H), 4.04 (d, J = 8.1 Hz, 1 H), 4.14-4.23 (m, 1 H), 4.53 (br d, J = 3.0 Hz, 1 H), 6.56-6.57 (m, 1 H), 6.57-6.59 (m, 1 H), 6.77 (d, J = 9.1 Hz, 1 H), 7.28 (d, J = 9.1 Hz, 1 H), 9.24-9.50 (m, 1 H) |
| 85 | 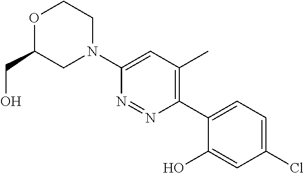 [SMILES: Cc1cc(N2CCO[C@H](CO)C2)nnc1-c1ccc(Cl)cc1O] | 336 0.98 B | 1H NMR (400 MHz, DMSO-d6, 30° C.) δ ppm 2.09 (s, 3 H), 2.70 (dd, J = 12.8, 9.9 Hz, 1 H), 2.96 (td, J = 12.3, 3.4 Hz, 1 H), 3.40-3.56 (m, 3 H), 3.60 (td, J = 11.6, 2.6 Hz, 1 H), 3.98 (br dd, J = 11.6, 2.1 Hz, 1 H), 4.13 (br d, J = 13.0 Hz, 1 H), 4.29 (br d, J = 12.8 Hz, 1 H), 4.75-4.85 (m, 1 H), 6.93-6.98 (m, 2 H), 7.15 (br s, 1 H), 7.18-7.22 (m, 1 H), 10.26 (br s, 1 H) |
| 86 | 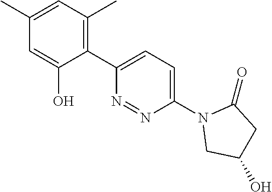 [SMILES: Cc1cc(C)c(-c2ccc(N3C[C@@H](O)CC3=O)nn2)c(O)c1] | 300.1 1.16 B | 1H NMR (400 MHz, DMSO-d6, 30° C.) δ ppm 2.01 (s, 3 H), 2.24 (s, 3 H), 2.41 (br d, J = 17.5 Hz, 1 H), 2.98 (dd, J = 17.3, 5.9 Hz, 1 H), 4.10 (br d, J = 11.8 Hz, 1 H), 4.21 (dd, J = 11.8, 5.0 Hz, 1 H), 4.43-4.54 (m, 1 H), 5.38 (d, J = 3.6 Hz, 1 H), 6.61 (br d, J = 3.6 Hz, 2 H), 7.63 (d, J = 9.3 Hz, 1 H), 8.56 (d, J = 9.3 Hz, 1 H), 9.39 (s, 1 H) |
| 87 | 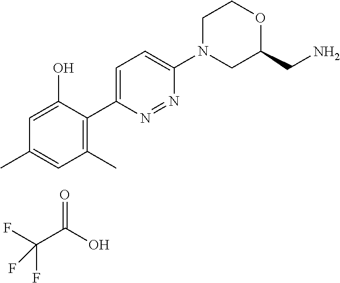 | 315.2 0.69-071 B | 1H NMR (400 MHz, DMSO-d6, 30° C.) δ ppm 2.03 (s, 3 H), 2.23 (s, 3 H), 2.79 (dd, J = 12.9, 10.5 Hz, 1 H), 2.87-3.19 (m, 3 H), 3.62-3.72 (m, 1 H), 3.74- 3.85 (m, 1 H), 4.05-4.18 (m, 2 H), 4.40 (br d, J = 12.6 Hz, 1 H), 6.57-6.58 (m, 1 H), 6.59-6.60 (m, 1 H), 7.31 (d, J = 9.5 Hz, 1 H), 7.41 (d, J = 9.5 Hz, 1 H), 7.92 (br s, 3 H), 9.39 (br s, 1 H) |
| 88 | 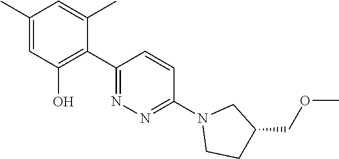 | 314 2.96 E | 1H NMR (400 MHz, DMSO-d6) δ ppm 9.41 (s, 1H), 7.28 (d, J = 9.2 Hz, 1H), 6.89 (s, 1H), 6.57 (d, J = 5.5 Hz, 2H), 3.67-3.56 (m, 2H), 3.51-3.45 (m, 1H), 3.43-3.36 (m, 2H), 3.30 (s, 3H), 3.24 (dd, J = 10.5, 6.6 Hz, 1H), 2.62 (m, 1H), 2.23 (s, 3H), 2.11 (h, J = 7.0 Hz, 1H), 2.04 (s, 3H), 1.83-1.73 (m, 1H). |
| 89 | 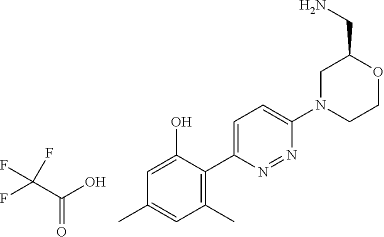 [SMILES: O=C(O)C(F)(F)F ; Cc1cc(C)c(-c2ccc(N3CCO[C@H](CN)C3)nn2)c(O)c1] | 315.2 0.72 B | 1H NMR (400 MHz, DMSO-d6, 30° C.) δ ppm 2.05 (s, 3 H), 2.24 (s, 3 H), 2.85 (dd, J = 12.8, 10.6 Hz, 1 H), 2.91-3.03 (m, 1 H), 3.06-3.17 (m, 2 H), 3.53- 3.90 (m partially hidden, 2 H), 4.06-4.19 (m, 2 H), 4.38 (br d, J = 12.5 Hz, 1 H), 6.56-6.67 (m, 2 H), 7.44-7.66 (m, 2 H), 7.94 (br s, 3 H), 9.23-9.92 (m, 1 H) |
| 90 | 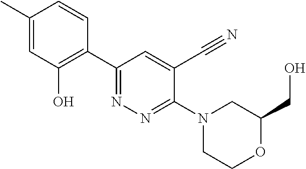 [SMILES: Cc1ccc(-c2cc(C#N)c(N3CCO[C@H](CO)C3)nn2)c(O)c1] | 327.1 1.45 B | 1H NMR (400 MHz, DMSO-d6, 30° C.) δ ppm 2.30 (s, 3 H) 3.01 (dd, J = 12.9, 10.4 Hz, 1 H) 3.19-3.29 (m, 1 H) 3.38-3.47 (m, 1 H) 3.48-3.57 (m, 1 H) 3.59-3.74 (m, 2 H) 3.95-4.03 (m, 1 H) 4.12 (br d, J = 12.3 Hz, 1 H) 4.28 (br d, J = 13.0 Hz, 1 H) 4.86 (t, J = 5.8 Hz, 1 H) 6.74-6.84 (m, 2 H) 7.81 (d, J = 8.0 Hz, 1 H) 8.70 (s, 1 H) 11.79 (s, 1 H) |
| 91 | 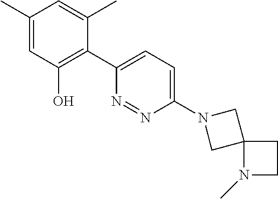 [SMILES: Cc1cc(C)c(-c2ccc(N3CC4(CCN4C)C3)nn2)c(O)c1] | 311.2 0.74 B | 1H NMR (400 MHz, DMSO-d6, 30° C.) δ ppm 2.01 (s, 3 H), 2.22 (s, 3 H), 2.28 (s, 3 H), 2.32 (t, J = 6.7 Hz, 2 H), 3.07 (t, J = 6.7 Hz, 2 H), 4.03 (d, J = 10.0 Hz, 2 H), 4.21 (d, J = 10.0 Hz, 2 H), 6.54-6.59 (m, 2 H), 6.82 (d, J = 9.1 Hz, 1 H), 7.29 (d, J = 9.1 Hz, 1 H), 9.33 (s, 1 H) |
| 92 | 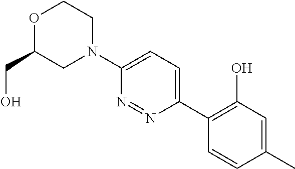 [SMILES: Cc1ccc(-c2ccc(N3CCO[C@H](CO)C3)nn2)c(O)c1] | 302.1 1.22 B | 1H NMR (400 MHz, DMSO-d6, 30° C.) δ ppm 2.29 (s, 3 H), 2.77 (dd, J = 12.9, 9.8 Hz, 1 H), 3.02 (ddd, J = 12.9, 11.8, 3.5 Hz, 1 H), 3.43-3.57 (m, 3 H), 3.60 (td, J = 11.6, 2.8 Hz, 1 H), 3.94-4.07 (m, 1 H), 4.10- 4.22 (m, 1 H), 4.32 (br d, J = 12.9 Hz, 1 H), 4.67-5.00 (m, 1 H), 6.71-6.82 (m, 2 H), 7.52 (d, J = 10.0 Hz, 1 H), 7.77 (d, J = 7.9 Hz, 1 H), 8.22 (d, J = 10.0 Hz, 1 H), 13.46 (br s, 1 H) |
| 93 | 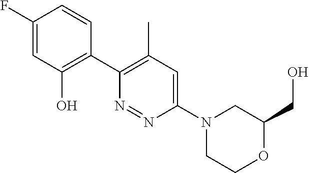 [SMILES: Cc1cc(N2CCO[C@H](CO)C2)nnc1-c1ccc(F)cc1O] | 320.1 0.86 B | 1H NMR (400 MHz, DMSO-d6, 30° C.) δ ppm 2.10 (s, 3 H), 2.69 (dd, J = 12.8, 9.9 Hz, 1 H), 2.95 (td, J = 12.3, 3.4 Hz, 1 H), 3.41-3.56 (m, 3 H), 3.60 (td, J = 11.6, 2.8 Hz, 1 H), 3.98 (dd, J = 11.5, 2.1 Hz, 1 H), 4.13 (br d, J = 12.9 Hz, 1 H), 4.29 (br d, J = 12.6 Hz, 1 H), 4.76-4.88 (m, 1 H), 6.67-6.77 (m, 2 H), 7.15 (s, 1 H), 7.17-7.27 (m, 1 H), 10.12-10.47 (m, 1 H) |
| 94 | 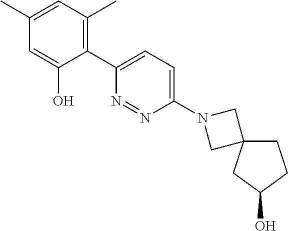 [SMILES: Cc1cc(C)c(-c2ccc(N3CC4(CC[C@@H](O)C4)C3)nn2)c(O)c1] | 326.2 0.93 B | 1H NMR (400 MHz, DMSO-d6, 30° C.) δ ppm 1.50- 1.61 (m, 1 H), 1.76-1.90 (m, 3 H), 2.01 (s, 3 H), 2.00-2.11 (m, 2 H), 2.22 (s, 3 H), 3.89-3.97 (m, 3 H), 4.03 (d, J = 8.0 Hz, 1 H), 4.13-4.22 (m, 1 H), 4.51 (d, J = 3.5 Hz, 1 H), 6.55-6.59 (m, 2 H), 6.76 (d, J = 9.2 Hz, 1 H), 7.28 (d, J = 9.2 Hz, 1 H), 9.33 (s, 1 H) |
| 95 | 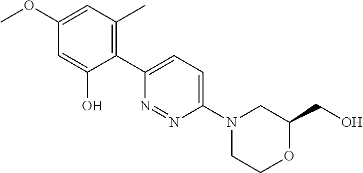 [SMILES: COc1cc(C)c(-c2ccc(N3CCO[C@H](CO)C3)nn2)c(O)c1] | 326.2 0.94 B | 1H NMR (400 MHz, DMSO-d6, 30° C.) δ ppm 1.50- 1.62 (m, 1 H), 1.76-1.89 (m, 3 H), 1.97-2.12 (m, 2 H), 2.01 (s, 3 H), 2.22 (s, 3 H), 3.90-3.97 (m, 3 H), 4.03 (d, J = 8.0 Hz, 1 H), 4.14-4.21 (m, 1 H), 4.51 (d, J = 3.5 Hz, 1 H), 6.54-6.60 (m, 2 H), 6.76 (d, J = 9.1 Hz, 1 H), 7.28 (d, J = 9.1 Hz, 1 H), 9.33 (s, 1 H) |
| 96 | 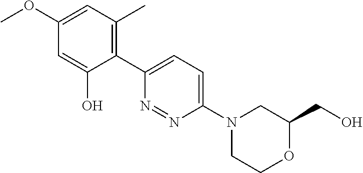 [SMILES: COc1cc(C)c(-c2ccc(N3CCO[C@H](CO)C3)nn2)c(O)c1] | 332.1 0.86 B | 1H NMR (400 MHz, DMSO-d6, 30° C.) δ ppm 2.06 (s, 3 H), 2.71 (dd, J = 12.8, 9.9 Hz, 1 H), 2.91-3.03 (m, 1 H), 3.42-3.57 (m, 3 H), 3.61 (td, J = 11.6, 2.8 Hz, 1 H), 3.72 (s, 3 H), 3.93-4.02 (m, 1 H), 4.11 (br d, J = 13.0 Hz, 1 H), 4.30 (br d, J = 12.6 Hz, 1 H), 4.79 (t, J = 5.6 Hz, 1 H), 6.34-6.37 (m, 2 H), 7.25 (d, J = 9.6 Hz, 1 H), 7.35 (d, J = 9.6 Hz, 1 H), 9.62 (s, 1 H) |
| 97 | 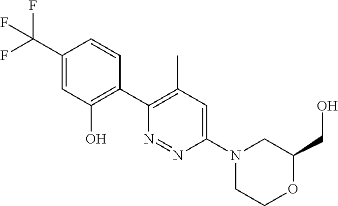 [SMILES: Cc1cc(N2CCO[C@H](CO)C2)nnc1-c1ccc(C(F)(F)F)cc1O] | 370.1 1.12 B | 1H NMR (400 MHz, DMSO-d6, 30° C.) δ ppm 2.10 (s, 3 H), 2.72 (dd, J = 12.8, 9.9 Hz, 1 H), 2.93-3.02 (m, 1 H), 3.41-3.56 (m, 3 H), 3.60 (td, J = 11.6, 2.8 Hz, 1 H), 3.96-4.03 (m, 1 H), 4.15 (br d, J = 13.0 Hz, 1 H), 4.31 (br d, J = 12.8 Hz, 1 H), 4.73-4.87 (m, 1 H), 7.17 (s, 1 H), 7.22 (br s, 1 H), 7.24 (br d, J = 8.0 Hz, 1 H), 7.41 (br d, J = 7.6 Hz, 1 H), 10.28-10.55 (m, 1 H) |
| 98 | 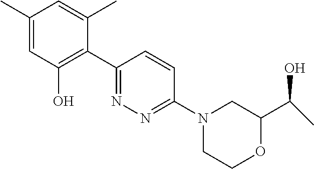 [SMILES: Cc1cc(C)c(-c2ccc(N3CCOC([C@H](C)O)C3)nn2)c(O)c1] | 330.4 2.53 E | 1H NMR (400 MHz, CDCl3) δ (ppm) : 7.53 (d, J = 9.6 Hz, 1H), 7.06 (d, J = 9.7 Hz, 1H), 6.76 (s, 1H), 6.65 (s, 1H), 4.29-4.22 (m, 1H), 4.21-4.14 (m, 1H), 4.14-4.08 (m, 1H), 4.01-3.88 (m, 1H), 3.84- 3.72 (m, 1H), 3.54-3.48 (m, 1H), 3.21-3.12 (m, 1H), 3.07 (dd, J = 12.8, 10.5 Hz, 1H), 2.38 (s, 3H), 2.30 (s, 3H), 2.01 (br. s, 1H), 1.30 (d, J = 6.5 Hz, 3H). (proton of phenol not visible) |
| 99 | 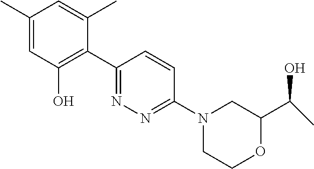 [SMILES: Cc1cc(C)c(-c2ccc(N3CCOC([C@H](C)O)C3)nn2)c(O)c1] | 330.4 2.5 E | 1H NMR (400 MHz, CDCl3) δ (ppm) : 10.97 (s, 1H), 7.54 (d, J = 9.7 Hz, 1H), 7.04 (d, J = 9.7 Hz, 1H), 6.76 (s, 1H), 6.65 (s, 1H), 4.28-4.21 (m, 1H), 4.18- 4.12 (m, 1H), 4.12-4.05 (m, 1H), 3.85-3.79 (m, 1H), 3.79-3.73 (m, 1H), 3.45-3.39 (m, 1H), 3.20- 3.11 (m, 1H), 2.98-2.91 (m, 1H), 2.48 (br. s, 1H), 2.38 (s, 3H), 2.30 (s, 3H), 1.29 (d, J = 6.3 Hz, 3H). |
| 102 | 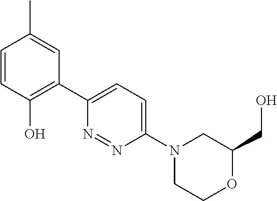 [SMILES: Cc1ccc(O)c(-c2ccc(N3CCO[C@H](CO)C3)nn2)c1] | 302.1 1.23 B | 1H NMR (400 MHz, DMSO-d6, 30° C.) δ ppm 2.29 (s, 3 H), 2.78 (dd, J = 12.9, 9.9 Hz, 1 H), 2.98-3.08 (m, 1 H), 3.41-3.57 (m, 3 H), 3.61 (td, J = 11.6, 2.8 Hz, 1 H), 3.98 (br dd, J = 11.6, 2.2 Hz, 1 H), 4.17 (br d, J = 13.0 Hz, 1 H), 4.34 (br d, J = 12.9 Hz, 1 H), 4.81 (br t, J = 4.7 Hz, 1 H), 6.83 (d, J = 8.3 Hz, 1 H), 7.08 (dd, J = 8.3, 1.5 Hz, 1 H), 7.53 (d, J = 9.9 Hz, 1 H), 7.71 (d, J = 1.5 Hz, 1 H), 8.24 (d, J = 9.9 Hz, 1 H), 13.18 (s, 1 H) |
| 103 | 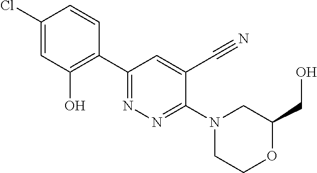 [SMILES: N#Cc1cc(-c2ccc(Cl)cc2O)nnc1N1CCO[C@H](CO)C1] | 347.1 1.52 B | 1H NMR (400 MHz, DMSO-d6, 30° C.) δ ppm 3.04 (dd, J = 13.0, 10.4 Hz, 1 H), 3.23-3.31 (m partially hidden, 1 H), 3.40-3.48 (m, 1 H), 3.50-3.57 (m, 1 H), 3.62 (ddd, J = 10.4, 5.3, 2.3 Hz, 1 H), 3.68 (td, J = 11.6, 2.5 Hz, 1 H), 4.00 (dd, J = 11.5, 1.5 Hz, 1 H), 4.18 (br dd, J = 13.0, 1.4 Hz, 1 H), 4.34 (br d, J = 13.0 Hz, 1 H), 4.86 (t, J = 5.7 Hz, 1 H), 7.01-7.12 (m, 2 H), 7.92 (d, J = 8.4 Hz, 1 H), 8.64 (s, 1 H), 11.91 (br s, 1 H) |
| 105 | 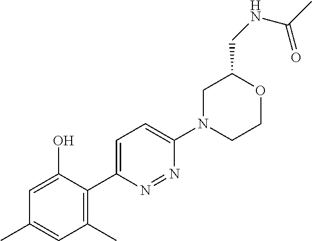 [SMILES: CC(=O)NC[C@H]1CN(c2ccc(-c3c(C)cc(C)cc3O)nn2)CCO1] | 357.2 0.96 B | 1H NMR (400 MHz, DMSO-d6, 30° C.) δ ppm 1.85 (s, 3 H), 2.03 (s, 3 H), 2.22 (s, 3 H), 2.67 (dd, J = 12.8, 10.6 Hz, 1 H), 2.97 (td, J = 12.2, 3.4 Hz, 1 H), 3.23 (t, J = 5.8 Hz, 2 H), 3.50-3.67 (m, 2 H), 4.00 (br dd, J = 11.5, 2.0 Hz, 1 H), 4.10 (br d, J = 12.9 Hz, 1 H), 4.28 (br d, J = 12.3 Hz, 1 H), 6.54-6.62 (m, 2 H), 7.28 (d, J = 9.5 Hz, 1 H), 7.35 (d, J = 9.5 Hz, 1 H), 8.03 (br t, J = 5.8 Hz, 1 H), 9.37 (s, 1 H) |
| 106 | 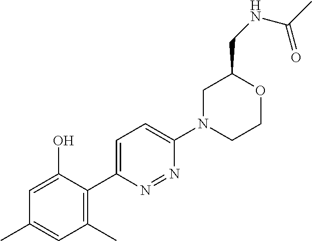 [SMILES: CC(=O)NC[C@@H]1CN(c2ccc(-c3c(C)cc(C)cc3O)nn2)CCO1] | 357.2 0.96 B | 1H NMR (400 MHz, DMSO-d6, 30° C.) δ ppm 1.85 (s, 3 H), 2.03 (s, 3 H), 2.22 (s, 3 H), 2.67 (dd, J = 12.8, 10.6 Hz, 1 H), 2.97 (td, J = 12.2, 3.4 Hz, 1 H), 3.23 (t, J = 5.9 Hz, 2 H), 3.51-3.70 (m, 2 H), 4.00 (br dd, J = 11.5, 2.0 Hz, 1 H), 4.10 (br d, J = 12.8 Hz, 1 H), 4.28 (br d, J = 12.4 Hz, 1 H), 6.55-6.62 (m, 2 H), 7.28 (d, J = 9.5 Hz, 1 H), 7.35 (d, J = 9.5 Hz, 1 H), 8.03 (t, J = 5.8 Hz, 1 H), 9.37 (s, 1 H) |
| 107 | 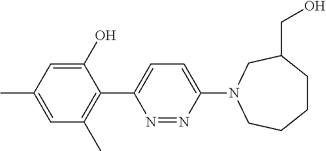 [SMILES: Cc1cc(C)c(-c2ccc(N3CCCCC(CO)C3)nn2)c(O)c1] | 328.2 0.98 B | 1H NMR (400 MHz, DMSO-d6, 30° C.) δ ppm 1.20- 1.41 (m, 2 H), 1.55-1.81 (m, 3 H), 1.85-1.99 (m, 2 H), 2.06 (s, 3 H), 2.23 (s, 3 H), 3.21 (br dd, J = 14.3, 10.1 Hz, 1 H), 3.28-3.42 (m, 2 H), 3.43-3.57 (m, 1 H), 3.89-4.08 (m, 2 H), 4.68 (br t, J = 5.0 Hz, 1 H), 6.57 (br s, 1 H), 6.58 (br s, 1 H), 7.07 (d, J = 9.4 Hz, 1 H), 7.28 (d, J = 9.4 Hz, 1 H), 9.46 (s, 1 H) |
| 108 | 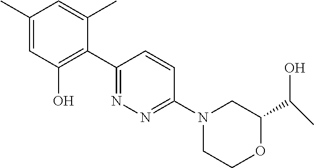 [SMILES: Cc1cc(C)c(-c2ccc(N3CCO[C@@H](C(C)O)C3)nn2)c(O)c1] | 330.4 2.39 E | ¹H NMR (500 MHz, CDCl₃) δ (ppm): 10.99 (s, 1H), 7.54 (d, J = 9.6 Hz, 1H), 7.04 (d, J = 9.6 Hz, 1H), 6.77 (s, 1H), 6.65 (s, 1H), 4.31-4.20 (m, 1H), 4.19- 4.11 (m, 1H), 4.12-4.06 (m, 1H), 3.84-3.79 (m, 1H), 3.78-3.74 (m, 1H), 3.48-3.37 (m, 1H), 3.16 (td, J = 12.3, 3.6 Hz, 1H), 2.95 (dd, J = 12.8, 10.8 Hz, 1H), 2.52-2.43 (m, 1H), 2.38 (s, 3H), 2.31 (s, 3H), 1.30 (d, J = 6.4 Hz, 3H). |
| 109 | 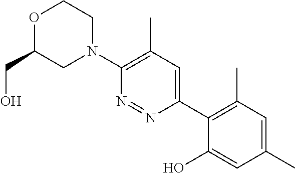 [SMILES: Cc1cc(C)c(-c2cc(C)c(N3CCO[C@H](CO)C3)nn2)c(O)c1] | 330.1 1.05 B | 1H NMR (500 MHz, DMSO-d6, 27° C.) δ ppm 2.00 (s, 3 H), 2.23 (s, 3 H), 2.30 (s, 3 H), 2.78 (dd, J = 12.1, 10.6 Hz, 1 H), 2.99 (td, J = 12.0, 2.5 Hz, 1 H), 3.40-3.47 (m, 2 H), 3.48-3.53 (m, 1 H), 3.55 (br d, J = 12.9 Hz, 1 H), 3.61-3.68 (m, 1 H), 3.69- 3.78 (m, 1 H), 3.94 (br d, J = 11.0 Hz, 1 H), 4.75 (t, J = 5.8 Hz, 1 H), 6.58 (br s, 1 H), 6.59 (br s, 1 H), 7.32 (s, 1 H), 9.37 (s, 1 H) |
| 110 | 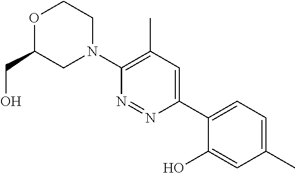 [SMILES: Cc1ccc(-c2cc(C)c(N3CCO[C@H](CO)C3)nn2)c(O)c1] | 316.1 1.36 B | 1H NMR (500 MHz, DMSO-d6, 27° C.) δ ppm 2.30 (s, 3 H), 2.40 (s, 3 H), 2.78 (dd, J = 12.5, 10.2 Hz, 1 H), 2.95-3.04 (m, 1 H), 3.41-3.55 (m, 3 H), 3.57- 3.68 (m, 2 H), 3.72 (td, J = 11.3, 1.9 Hz, 1 H), 3.93 (br d, J = 11.3 Hz, 1 H), 4.73-4.82 (m, 1 H), 6.73-6.81 (m, 2 H), 7.82 (d, J = 7.9 Hz, 1 H), 8.23 (s, 1 H), 13.52 (s, 1 H) |
| 111 | 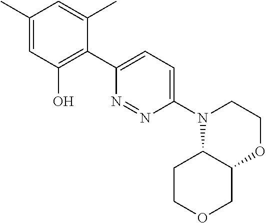 [SMILES: Cc1cc(C)c(-c2ccc(N3CCO[C@@H]4COCC[C@@H]43)nn2)c(O)c1] | 342.4 2.63 E | ¹H NMR (500 MHz, DMSO-d6) δ (ppm): 10.89 (br. s, 1H), 7.53 (d, J = 9.6 Hz, 1H), 6.98 (d, J = 9.7 Hz, 1H), 6.77 (s, 1H), 6.65 (s, 1H), 4.64 (d, J = 12.0 Hz, 1H), 4.20 (dd, J = 11.3, 3.9 Hz, 1H), 4.16-4.01 (m, 2H), 3.84-3.73 (m, 2H), 3.67 (s, 1H), 3.65-3.62 (m, 1H), 3.57 (td, J = 12.1, 2.1 Hz, 1H), 3.41 (td, J = 12.7, 3.9 Hz, 1H), 2.38 (s, 3H), 2.31 (s, 3H), 2.26 (dd, J = 12.5, 4.9 Hz, 1H), 1.68 (d, J = 13.1 Hz, 1H). |
| 112 | 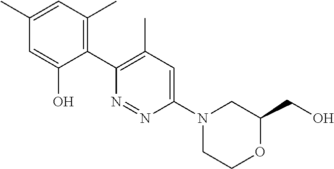 [SMILES: Cc1cc(C)c(-c2nnc(N3CCO[C@H](CO)C3)cc2C)c(O)c1] | 330.2 0.92 B | 1H NMR (400 MHz, DMSO-d6, 30° C.) δ ppm 1.85 (s, 3 H), 1.95 (br s, 3 H), 2.23 (s, 3 H), 2.68 (dd, J = 12.6, 9.8 Hz, 1 H), 2.87-3.02 (m, 1 H), 3.42-3.56 (m, 3 H), 3.61 (td, J = 11.6, 2.8 Hz, 1 H), 3.94-4.02 (m, 1 H), 4.08-4.17 (m, 1 H), 4.24-4.36 (m, 1 H), 4.74-4.85 (m, 1 H), 6.49-6.65 (m, 2 H), 7.14 (s, 1 H), 9.12 (s, 1 H) |
| 113 | 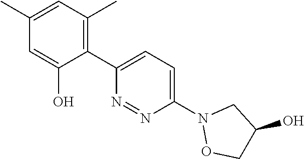 [SMILES: Cc1cc(C)c(-c2ccc(N3C[C@@H](O)CO3)nn2)c(O)c1] | 288.1 1.04 B | 1H NMR (400 MHz, DMSO-d6, 30° C.) δ ppm 2.01 (s, 3 H), 2.23 (s, 3 H), 3.79-3.86 (m, 2 H), 3.90 (dd, J = 8.9, 5.1 Hz, 1 H), 4.11 (dd, J = 11.4, 6.1 Hz, 1 H), 4.71-4.80 (m, 1 H), 5.35 (br s, 1 H), 6.58 (br s, 1 H), 6.60 (br s, 1 H), 7.42 (d, J = 9.1 Hz, 1 H), 7.47 (d, J = 9.1 Hz, 1 H), 9.30-9.46 (m, 1 H) |
| 114 | 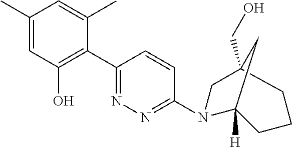 [SMILES: Cc1cc(C)c(-c2ccc(N3C[C@]4(CO)CCC[C@H]3C4)nn2)c(O)c1] | 340 0.96 U | 1H NMR (400 MHz, DMSO-d6, 30° C.) δ ppm 1.27- 1.50 (m, 4 H) 1.55-1.66 (m, 2 H) 1.82-2.01 (m, 2 H) 2.05 (s, 3 H) 2.22 (s, 3 H) 3.33-3.49 (m, 4 H) 4.30-4.50 (m, 1 H) 4.67-4.72 (m, 1 H) 6.56 (br s, 1 H) 6.57 (br s, 1 H) 6.84 (br d, J = 9.0 Hz, 1 H) 7.24 (d, J = 9.0 Hz, 1 H) 9.44 (br s, 1 H) |
| 115 | 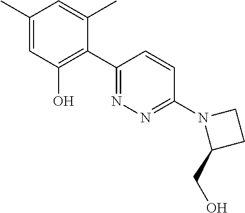 [SMILES: Cc1cc(C)c(-c2ccc(N3CC[C@H]3CO)nn2)c(O)c1] | 286 0.86 U | 1H NMR (400 MHz, DMSO-d6, 30° C.) δ ppm 2.01 (s, 3 H) 2.20-2.28 (m, 1 H) 2.21 (s, 3 H) 2.29-2.43 (m, 1 H) 3.65-3.76 (m, 2 H) 3.87-4.02 (m, 2 H) 4.34-4.43 (m, 1 H) 5.19 (br t, J = 5.3 Hz, 1 H) 6.56 (br s, 1 H) 6.57 (br s, 1 H) 6.97 (d, J = 9.3 Hz, 1 H) 7.29 (d, J = 9.3 Hz, 1 H) 9.35 (s, 1 H) |
| 116 | 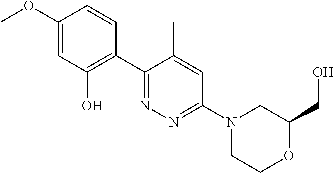 [SMILES: COc1ccc(-c2nnc(N3CCO[C@H](CO)C3)cc2C)c(O)c1] | 332 0.83 U | 1H NMR (400 MHz, DMSO-d6, 30° C.) δ ppm 2.13 (s, 3 H) 2.64-2.73 (m, 1 H) 2.88-3.00 (m, 1 H) 3.43- 3.63 (m, 4 H) 3.75 (s, 3 H) 3.94-4.05 (m, 1 H) 4.12 (br d, J = 12.7 Hz, 1 H) 4.27 (br d, J = 12.7 Hz, 1 H) 4.81 (br t, J = 5.4 Hz, 1 H) 6.45-6.53 (m, 2 H) 7.08- 7.16 (m, 2 H) 9.97 (br s, 1 H) |
| 117 | 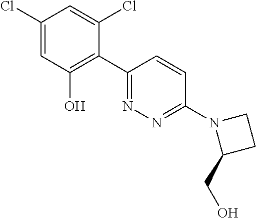 [SMILES: OC[C@@H]1CCN1c1ccc(-c2c(O)cc(Cl)cc2Cl)nn1] | 326 1.06 U | 1H NMR (400 MHz, DMSO-d6, 30° C.) δ ppm 2.19- 2.30 (m, 1 H) 2.31-2.45 (m, 1 H) 3.66-3.77 (m, 2 H) 3.91-4.04 (m, 2 H) 4.38-4.46 (m, 1 H) 5.15 (br t, J = 5.0 Hz, 1 H) 6.95 (d, J = 2.0 Hz, 1 H) 7.00 (d, J = 9.4 Hz, 1 H) 7.12 (d, J = 2.0 Hz, 1 H) 7.34 (d, J = 9.4 Hz, 1 H) 9.77-11.05 (m, 1 H) |
| 118 | 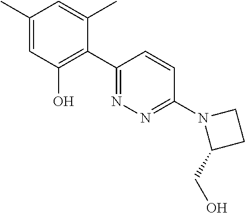 [SMILES: Cc1cc(C)c(-c2ccc(N3CC[C@@H]3CO)nn2)c(O)c1] | 286 0.86 U | 1H NMR (400 MHz, DMSO-d6, 30° C.) δ ppm 2.02 (s, 3 H) 2.17-2.29 (m, 1 H) 2.22 (s, 3 H) 2.29-2.40 (m, 1 H) 3.67-3.75 (m, 2 H) 3.91-3.98 (m, 2 H) 4.35-4.43 (m, 1 H) 5.18 (br t, J = 5.3 Hz, 1 H) 6.56 (br s, 1 H) 6.57 (br s, 1 H) 6.97 (d, J = 9.4 Hz, 1 H) 7.29 (d, J = 9.4 Hz, 1 H) 9.34 (s, 1 H) |
| 119 | 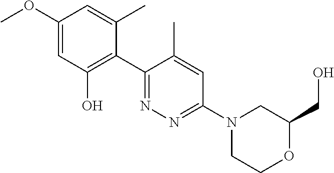 [SMILES: COc1cc(C)c(-c2nnc(N3CCO[C@H](CO)C3)cc2C)c(O)c1] | 346 0.86 U | 1H NMR (500 MHz, DMSO-d6, 25° C.) δ ppm 1.86 (s, 3 H) 1.96 (s, 3 H) 2.67 (dd, J = 12.7, 10.0 Hz, 1 H) 2.90-2.97 (m, 1 H) 3.42-3.49 (m, 1 H) 3.49- 3.56 (m, 2 H) 3.60 (td, J = 11.6, 2.6 Hz, 1 H) 3.72 (s, 3 H) 3.98 (br d, J = 11.6 Hz, 1 H) 4.08-4.16 (m, 1 H) 4.24-4.31 (m, 1 H) 4.77-4.87 (m, 1 H) 6.33 (d, J = 2.2 Hz, 1 H) 6.36 (d, J = 2.2 Hz, 1 H) 7.14 (s, 1 H) 9.33 (br s, 1 H) |
| 120 | 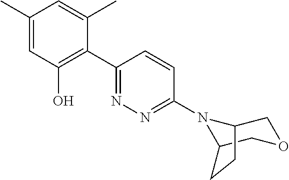 [SMILES: Cc1cc(C)c(-c2ccc(N3C4CCC3COC4)nn2)c(O)c1] | 312 1.01 U | 1H NMR (500 MHz, DMSO-d6, 25° C.) δ ppm 1.88- 2.05 (m, 4 H) 2.04 (s, 3 H) 2.22 (s, 3 H) 3.57 (d, J = 11.0 Hz, 2 H) 3.70 (d, J = 11.0 Hz, 2 H) 4.58- 4.62 (m, 2 H) 6.57 (br s, 1 H) 6.59 (br s, 1 H) 7.22 (d, J = 9.4 Hz, 1 H) 7.33 (d, J = 9.4 Hz, 1 H) 9.42 (br s, 1 H) |
| 121 | 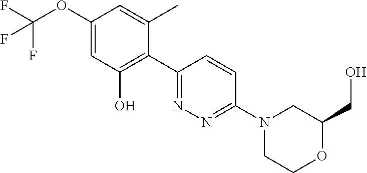 [SMILES: Cc1cc(OC(F)(F)F)cc(O)c1-c1ccc(N2CCO[C@H](CO)C2)nn1] | 386.3 2.57 E | 1H NMR (500 MHz, CDCl3) δ (ppm) : 11.47 (s, 1H), 7.54 (d, J = 9.6 Hz, 1H), 7.06 (d, J = 9.7 Hz, 1H), 6.81 (s, 1H), 6.67 (s, 1H), 4.23 (d, J = 12.8 Hz, 1H), 4.18-4.11 (m, 2H), 3.85-3.69 (m, 4H), 3.20 (td, J = 12.3, 3.6 Hz, 1H), 3.03 (dd, J = 12.8, 10.1 Hz, 1H), 2.43 (s, 3H), 1.93 (s, 1H). |
| 122 | 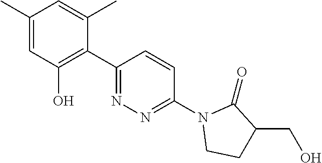 [SMILES: Cc1cc(C)c(-c2ccc(N3CCC(CO)C3=O)nn2)c(O)c1] | 314 1.22 U | 1H NMR (400 MHz, DMSO-d6, 30° C.) δ ppm 2.01 (s, 3 H) 2.08-2.20 (m, 1 H) 2.23-2.30 (m, 1 H) 2.24 (s, 3 H) 2.73-2.94 (m, 1 H) 3.63-3.72 (m, 1 H) 3.72- 3.82 (m, 1 H) 3.96-4.10 (m, 1 H) 4.11-4.23 (m, 1 H) 4.85 (t, J = 5.3 Hz, 1 H) 6.60 (br s, 1 H) 6.61 (br s, 1 H) 7.63 (d, J = 9.5 Hz, 1 H) 8.56 (d, J = 9.5 Hz, 1 H) 9.39 (br s, 1 H) |
| 123 | 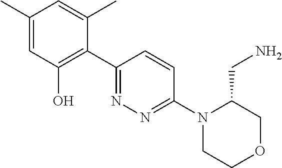 [SMILES: Cc1cc(C)c(-c2ccc(N3CCOC[C@H]3CN)nn2)c(O)c1] | 315 0.79 U | 1H NMR (400 MHz, DMSO-d6, 30° C.) δ ppm 2.06 (s, 3 H) 2.23 (s, 3 H) 3.10-3.40 (m, 3 H) 3.50-3.60 (m partially hidden, 1 H) 3.62-3.70 (m, 1 H) 3.90- 4.06 (m, 3 H) 4.74 (br t, J = 7.0 Hz, 1 H) 6.58-6.62 (m, 2 H) 7.35-7.55 (m, 2 H) 7.89 (br s, 3 H) 9.35- 9.60 (m, 1 H) |
| 124 | 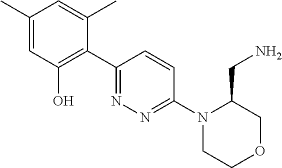 [SMILES: Cc1cc(C)c(-c2ccc(N3CCOC[C@@H]3CN)nn2)c(O)c1] | 315 0.79 U | 1H NMR (400 MHz, DMSO-d6, 30° C.) δ ppm 2.04 (s, 3 H) 2.22 (s, 3 H) 2.74 (dd, J = 12.4, 5.5 Hz, 1 H) 3.01 (dd, J = 12.4, 9.0 Hz, 1 H) 3.17 (td, J = 12.9, 3.5 Hz, 1 H) 3.47-3.59 (m, 2 H) 3.94 (dd, J = 11.5, 3.5 Hz, 1 H) 4.08-4.17 (m, 3 H) 6.57 (br s, 1 H) 6.58 (br s, 1 H) 7.24 (d, J = 9.5 Hz, 1 H) 7.33 (d, J = 9.5 Hz, 1 H) 9.25-9.50 (m, 1 H) |
| 125 | 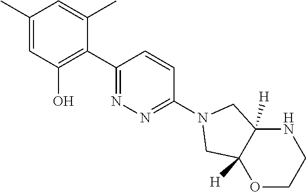 [SMILES: Cc1cc(C)c(-c2ccc(N3C[C@H]4NCCO[C@@H]4C3)nn2)c(O)c1] | 326.9 4.52 F | 1H NMR (400 MHz, CDCl3) δ (ppm): 7.42 (d, J = 9.5 Hz, 1H), 6.80-6.62 (m, 2H), 6.57 (s, 1H), 4.05- 3.80 (m, 3H), 3.75 (td, J = 11.7, 3.0 Hz, 1H), 3.72- 3.62 (m, 1H), 3.32 (t, J = 9.5 Hz, 1H), 3.25 (t, J = 10.1 Hz, 1H), 3.12-3.00 (m, 2H), 2.99-2.89 (m, 1H), 2.29 (s, 3H), 2.23 (s, 3H). (OH and NH not visible) |
| 126 | 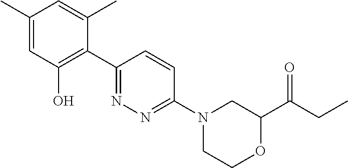 [SMILES: CCC(=O)C1CN(c2ccc(-c3c(C)cc(C)cc3O)nn2)CCO1] | 342.3 2.81 E | 1H NMR (500 MHz, DMSO-d6) δ (ppm): 9.37 (s, 1H), 7.35 (q, J = 9.4 Hz, 2H), 6.58 (d, J = 5.7 Hz, 2H), 4.40-4.33 (m, 1H), 4.19 (dd, J = 9.9, 3.2 Hz, 1H), 4.11-4.02 (m, 2H), 3.77-3.68 (m, 1H), 3.12 (ddd, J = 13.8, 10.9, 3.7 Hz, 1H), 3.02 (dd, J = 13.0, 10.0 Hz, 1H), 2.67 (qt, J = 6.9, 3.5 Hz, 2H), 2.23 (s, 3H), 2.03 (s, 3H), 0.97 (t, J = 7.2 Hz, 3H). |
| 127 | 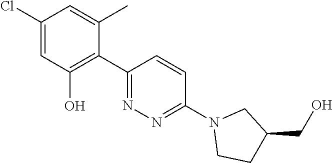 [SMILES: Cc1cc(Cl)cc(O)c1-c1ccc(N2CC[C@H](CO)C2)nn1] | 320 0.90 U | 1H NMR (400 MHz, DMSO-d6, 30° C.) δ ppm 1.74- 1.85 (m, 1 H) 2.01-2.11 (m, 1 H) 2.05 (s, 3 H) 2.43- 2.48 (m, 1 H) 3.25-3.66 (m partially hidden, 1 H) 3.41-3.51 (m, 3 H) 3.54-3.62 (m, 2 H) 4.65-4.80 (m, 1 H) 6.80-6.844 (m, 2 H) 6.87 (d, J = 9.5 Hz, 1 H) 7.28 (d, J = 9.5 Hz, 1 H) 9.90-10.20 (m, 1 H) |
| 128 | 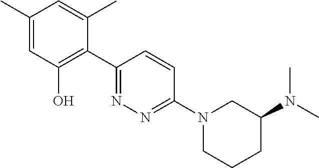 | 327 0.77 U | 1H NMR (400 MHz, DMSO-d6, 30° C.) δ ppm 1.41- 1.54 (m, 2 H) 1.76-1.84 (m, 1 H) 1.89-1.98 (m, 1 H) 2.04 (s, 3 H) 2.22 (s, 3 H) 2.22-2.26 (m, 1 H) 2.27 (s, 6 H) 2.79-2.94 (m, 2 H) 4.22 (br d, J = 12.7 Hz, 1 H) 4.46 (br d, J = 12.7 Hz, 1 H) 6.57 (br s, 1 H) 6.58 (br s, 1 H) 7.25 (d, J = 9.5 Hz, 1 H) 7.28 (d, J = 9.5 Hz, 1 H) 9.41 (s, 1 H) |
| 129 | 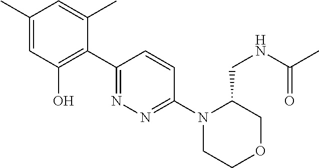 [SMILES: CC(=O)NC[C@@H]1COCCN1c1ccc(-c2c(C)cc(C)cc2O)nn1] | 357 0.80 U | 1H NMR (400 MHz, DMSO-d6, 30° C.) 8 ppm 1.65 (s, 3 H) 2.03 (s, 3 H) 2.22 (s, 3 H) 3.20-3.30 (m partially hidden, 1 H) 3.36 (br t, J = 6.2 Hz, 2 H) 3.51 (br td, J = 11.5, 3.0 Hz, 1 H) 3.59 (dd, J = 11.5, 3.0 Hz, 1 H) 3.89 (d, J = 11.5 Hz, 1 H) 3.97 (br dd, J = 11.5, 3.0 Hz, 1 H) 4.16-4.27 (m, 2 H) 6.56 (br s, 1 H) 6.57 (br s, 1 H) 7.24 (d, J = 9.5 Hz, 1 H) 7.31 (d, J = 9.5 Hz, 1 H) 8.00 (t, J = 6.2 Hz, 1 H) 9.37 (br s, 1 H) |
| 130 | 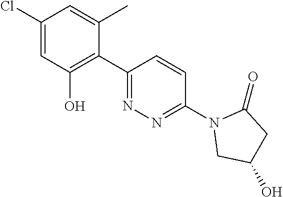 [SMILES: Cc1cc(Cl)cc(O)c1-c1ccc(N2C[C@@H](O)CC2=O)nn1] | 320 1.13 U | 1H NMR (400 MHz, DMSO-d6, 30° C.) δ ppm 2.04 (s, 3 H) 2.42 (br d, J = 17.5 Hz, 1 H) 2.98 (dd, J = 17.5, 6.0 Hz, 1 H) 4.10 (br d, J = 11.5 Hz, 1 H) 4.22 (dd, J = 11.5, 5.0 Hz, 1 H) 4.44-4.50 (m, 1 H) 5.38 (d, J = 3.5 Hz, 1 H) 6.84 (br s, 1 H) 6.87 (br s, 1 H) 7.67 (d, J = 9.5 Hz, 1 H) 8.59 (d, J = 9.5 Hz, 1 H) 9.95-10.10 (m, 1 H) |
| 131 | 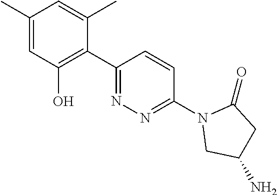 [SMILES: Cc1cc(C)c(-c2ccc(N3C[C@@H](N)CC3=O)nn2)c(O)c1] | 299 0.82 U | 1H NMR (400 MHz, DMSO-d6, 30° C.) δ ppm 1.97- 2.05 (m, 2 H) 2.01 (s, 3 H) 2.24 (s, 3 H) 2.30 (br dd, J = 17.0, 2.9 Hz, 1 H) 2.86 (dd, J = 17.0, 6.5 Hz, 1 H) 3.66-3.78 (m, 1 H) 3.88 (br dd, J = 11.0, 2.9 Hz, 1 H) 4.18 (dd, J = 11.0, 6.0 Hz, 1 H) 6.60 (br s, 1 H) 6.61 (br s, 1 H) 7.62 (d, J = 9.5 Hz, 1 H) 8.55 (d, J = 9.5 Hz, 1 H) 9.38 (br s, 1 H) |
| 132 | 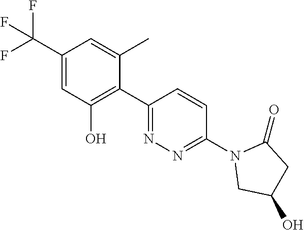 [SMILES: Cc1cc(C(F)(F)F)cc(O)c1-c1ccc(N2C[C@H](O)CC2=O)nn1] | 354 1.33 U | 1H NMR (400 MHz, DMSO-d6, 30° C.) δ ppm 2.11 (s, 3 H) 2.42 (br d, J = 17.5 Hz, 1 H) 2.99 (dd, J = 17.5, 6.0 Hz, 1 H) 4.11 (br d, J = 11.5 Hz, 1 H) 4.23 (dd, J = 11.5, 5.0 Hz, 1 H) 4.45-4.50 (m, 1 H) 4.95-5.65 (m, 1 H) 7.10 (br s, 1 H) 7.16 (br s, 1 H) 7.72 (d, J = 9.5 Hz, 1 H) 8.63 (d, J = 9.5 Hz, 1 H) 10.24 (s, 1 H) |
| 133 | 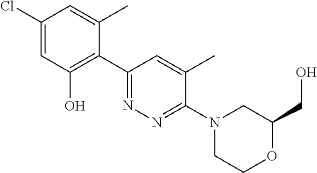 [SMILES: Cc1cc(-c2c(C)cc(Cl)cc2O)nnc1N1CCO[C@H](CO)C1] | 350 1.20 U | 1H NMR (400 MHz, DMSO-d6, 30° C.) δ ppm 2.03 (s, 3 H) 2.32 (s, 3 H) 2.74-2.85 (m, 1 H) 2.96-3.06 (m, 1 H) 3.42-3.61 (m, 4 H) 3.62-3.69 (m, 1 H) 3.73 (td, J = 11.3, 2.5 Hz, 1 H) 3.90-3.99 (m, 1 H) 4.74 (br t, J = 5.4 Hz, 1 H) 6.84 (br s, 1 H) 6.86 (br s, 1 H) 7.35 (s, 1 H) 9.96 (br s, 1 H) |
| 134 | 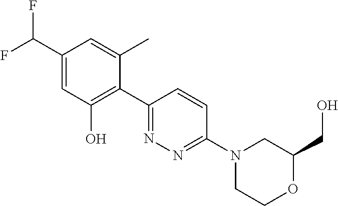 [SMILES: Cc1cc(C(F)F)cc(O)c1-c1ccc(N2CCO[C@H](CO)C2)nn1] | 352 1.01 U | 1H NMR (400 MHz, DMSO-d6, 30° C.) δ ppm 2.10 (s, 3 H) 2.73 (br dd, J = 12.7, 10.0 Hz, 1 H) 2.99 (td, J = 12.7, 2.6 Hz, 1 H) 3.41-3.58 (m, 3 H) 3.62 (td, J = 11.6, 2.6 Hz, 1 H) 4.00 (dd, J = 11.6, 2.6 Hz, 1 H) 4.15 (br d, J = 12.7 Hz, 1 H) 4.34 (br d, J = 12.7 Hz, 1 H) 4.82 (t, J = 5.4 Hz, 1 H) 6.79-7.11 (m, 3 H) 7.30 (d, J = 9.5 Hz, 1 H) 7.39 (d, J = 9.5 Hz, 1 H) 9.88 (s, 1 H) |
| 135 | 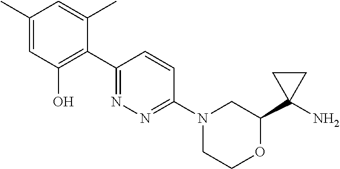 [SMILES: Cc1cc(C)c(-c2ccc(N3CCO[C@H](C4(N)CC4)C3)nn2)c(O)c1] | 341 0.79 U | 1H NMR (400 MHz, DMSO-d6, 30° C.) δ ppm 0.41- 0.62 (m, 4 H) 2.03 (s, 3 H) 2.05-2.30 (m, 2H) 2.22 (s, 3 H) 2.84-3.00 (m, 2 H) 3.09 (dd, J = 11.5, 2.5 Hz, 1 H) 3.59 (td, J = 11.5, 2.5 Hz, 1 H) 4.01 (dd, J = 11.5, 2.5 Hz, 1 H) 4.15 (br d, J = 12.0 Hz, 1 H) 4.33 (br d, J = 12.0 Hz, 1 H) 6.57 (br s, 1 H) 6.58 (br s, 1 H) 7.30 (d, J = 9.3 Hz, 1 H) 7.35 (d, J = 9.3 Hz, 1 H) 9.38 (s, 1 H) |
| 136 | 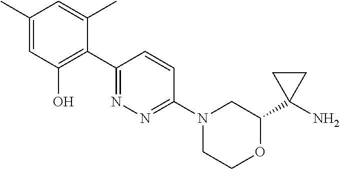 [SMILES: Cc1cc(C)c(-c2ccc(N3CCO[C@@H](C4(N)CC4)C3)nn2)c(O)c1] | 341 0.79 U | 1H NMR (400 MHz, DMSO-d6, 30° C.) δ ppm 0.41- 0.62 (m, 4 H) 1.90-2.20 (m, 2H) 2.03 (s, 3 H) 2.22 (s, 3 H) 2.84-3.00 (m, 2 H) 3.09 (dd, J = 11.5, 2.5 Hz, 1 H) 3.59 (td, J = 11.5, 2.5 Hz, 1 H) 4.01 (dd, J = 11.5, 2.5 Hz, 1 H) 4.15 (br d, J = 12.0 Hz, 1 H) 4.33 (br d, J = 12.0 Hz, 1 H) 6.57 (br s, 1 H) 6.58 (br s, 1 H) 7.30 (d, J = 9.3 Hz, 1 H) 7.35 (d, J = 9.3 Hz, 1 H) 9.38 (s, 1 H) |
| 137 | 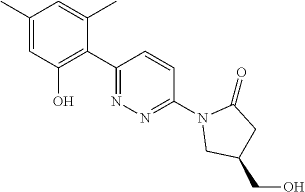 [SMILES: Cc1cc(C)c(-c2ccc(N3C[C@H](CO)CC3=O)nn2)c(O)c1] | 314 1.20 U | 1H NMR (400 MHz, DMSO-d6, 30° C.) δ ppm 2.01 (s, 3 H) 2.24 (s, 3 H) 2.44 (dd, J = 17.0, 5.5 Hz, 1 H) 2.54-2.63 (m, 1 H) 2.75 (dd, J = 17.0, 8.3 Hz, 1 H) 3.47-3.54 (m, 2 H) 3.97 (dd, J = 11.3, 5.5 Hz, 1 H) 4.20 (dd, J = 11.3, 8.3 Hz, 1 H) 4.87 (t, J = 5.5 Hz, 1 H) 6.60 (br s, 1 H) 6.61 (br s, 1 H) 7.62 (d, J = 9.3 Hz, 1 H) 8.54 (d, J = 9.3 Hz, 1 H) 9.39 (s, 1 H) |
| 138 | 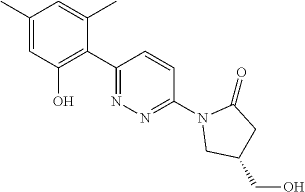 [SMILES: Cc1cc(C)c(-c2ccc(N3C[C@@H](CO)CC3=O)nn2)c(O)c1] | 314 1.20 U | 1H NMR (400 MHz, DMSO-d6, 30° C.) δ ppm 2.01 (s, 3 H) 2.24 (s, 3 H) 2.44 (dd, J = 17.0, 5.5 Hz, 1 H) 2.54-2.64 (m, 1 H) 2.74 (dd, J = 17.0, 8.3 Hz, 1 H) 3.47-3.55 (m, 2 H) 3.97 (dd, J = 11.3, 5.5 Hz, 1 H) 4.20 (dd, J = 11.3, 8.3 Hz, 1 H) 4.87 (t, J = 5.5 Hz, 1 H) 6.60 (br s, 1 H) 6.61 (br s, 1 H) 7.62 (d, J = 9.3 Hz, 1 H) 8.54 (d, J = 9.3 Hz, 1 H) 9.39 (s, 1 H) |
| 139 | 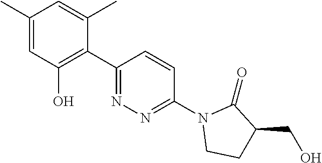 [SMILES: Cc1cc(C)c(-c2ccc(N3CC[C@H](CO)C3=O)nn2)c(O)c1] | 314 1.21 U | 1H NMR (400 MHz, DMSO-d6, 30° C.) δ ppm 2.01 (s, 3 H) 2.09-2.20 (m, 1 H) 2.21-2.30 (m, 1 H) 2.24 (s, 3 H) 2.80-2.88 (m, 1 H) 3.62-3.72 (m, 1 H) 3.72- 3.83 (m, 1 H) 3.97-4.09 (m, 1 H) 4.13-4.21 (m, 1 H) 4.85 (t, J = 5.3 Hz, 1 H) 6.60 (br s, 1 H) 6.62 (br s, 1 H) 7.63 (d, J = 9.0 Hz, 1 H) 8.56 (d, J = 9.0 Hz, 1 H) 9.39 (s, 1 H) |
| 140 | 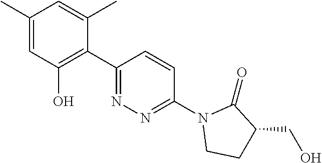 [SMILES: Cc1cc(C)c(-c2ccc(N3CC[C@@H](CO)C3=O)nn2)c(O)c1] | 314 1.22 U | 1H NMR (400 MHz, DMSO-d6, 30° C.) δ ppm 2.01 (s, 3 H) 2.09-2.20 (m, 1 H) 2.21-2.30 (m, 1 H) 2.24 (s, 3 H) 2.80-2.88 (m, 1 H) 3.62-3.72 (m, 1 H) 3.72- 3.83 (m, 1 H) 3.97-4.09 (m, 1 H) 4.13-4.21 (m, 1 H) 4.85 (t, J = 5.3 Hz, 1 H) 6.60 (br s, 1 H) 6.62 (br s, 1 H) 7.63 (d, J = 9.0 Hz, 1 H) 8.56 (d, J = 9.0 Hz, 1 H) 9.39 (s, 1 H) |
| 141 | 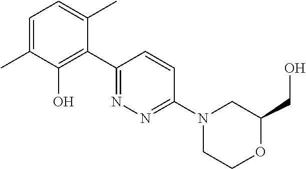 [SMILES: Cc1ccc(C)c(-c2ccc(N3CCO[C@H](CO)C3)nn2)c1O] | 316.3 2.42 E | 1H NMR (400 MHz, CDCl3) δ (ppm): 10.97 (s, 1H), 7.55 (d, J = 9.6 Hz, 1H), 7.11-7.01 (m, 2H), 6.75 (d, J = 7.6 Hz, 1H), 4.24 (d, J = 12.8 Hz, 1H), 4.16 (ddt, J = 11.5, 3.4, 2.0 Hz, 2H), 3.89-3.68 (m, 4H), 3.27-3.15 (m, 1H), 3.04 (dd, J = 12.8, 9.9 Hz, 1H), 2.39 (s, 3H), 2.31 (s, 3H), 2.00 (s, 1H). |
| 142 | 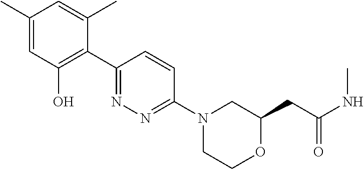 | 316.4 2.25 E | 1H-NMR (500 MHz, DMSO-d6) δ (ppm): 9.31 (s, 1H), 7.80 (d, J = 4.8 Hz, 1H), 7.29 (d, J = 9.4 Hz, 1H), 7.19 (d, J = 9.5 Hz, 1H), 6.57-6.48 (m, 2H), 4.22 (d, J = 12.7 Hz, 1H), 4.03 (d, J = 12.7 Hz, 1H), 3.94-3.85 (m, 1H), 3.81 (qd, J = 7.3, 2.5 Hz, 1H), 3.59-3.46 (m, 1H), 2.89 (td, J = 12.3, 3.5 Hz, 1H), 2.68-2.61 (m, 1H), 2.53 (d, J = 4.6 Hz, 3H), 2.31- 2.23 (m, 2H), 2.16 (s, 3H), 1.96 (s, 3H). |
| 143 | 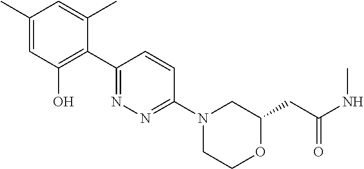 | 357.4 2.25 E | 1H-NMR (400 MHz, DMSO-d6) δ (ppm): 9.31 (s, 1H), 7.80 (d, J = 4.8 Hz, 1H), 7.29 (d, J = 9.4 Hz, 1H), 7.19 (d, J = 9.5 Hz, 1H), 6.53 - 6.48 (m, 2H), 4.22 (dd, J = 12.8, 2.4 Hz, 1H), 4.03 (d, J = 12.8 Hz, 1H), 3.93-3.85 (m, 1H), 3.81 (dtd, J = 13.0, 6.4, 2.5 Hz, 1H), 3.52 (td, J = 11.6, 2.7 Hz, 1H), 2.89 (td, J = 12.3, 3.5 Hz, 1H), 2.68-2.61 (m, 1H), 2.53 (d, J = 4.6 Hz, 3H), 2.28 (d, J = 3.2 Hz, 2H), 2.15 (s, 3H), 1.96 (s, 3H). |
| 144 | 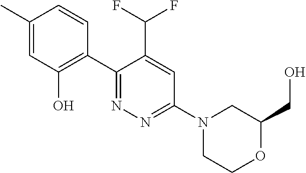 [SMILES: Cc1ccc(-c2nnc(N3CCO[C@H](CO)C3)cc2C(F)F)c(O)c1] | 352 1.23 U | 1H NMR (400 MHz, DMSO-d6, 30° C.) δ ppm 2.29 (s, 3 H) 2.79 (dd, J = 12.7, 10.0 Hz, 1 H) 3.03 (td, J = 12.7, 2.6 Hz, 1 H) 3.44 - 3.66 (m, 4 H) 3.99 (dd, J = 11.6, 2.6 Hz, 1 H) 4.24 (br d, J = 12.7 Hz, 1 H) 4.38 (br d, J = 12.7 Hz, 1 H) 4.82 (t, J = 5.4 Hz, 1 H) 6.60-6.92 (m, 3 H) 7.15 (d, J = 7.8 Hz, 1 H) 7.38 (s, 1 H) 9.82 (br s, 1 H) |
| 145 | 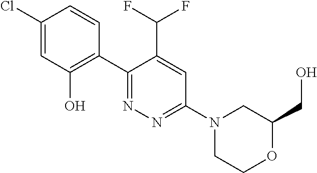 [SMILES: OC[C@@H]1CN(c2cc(C(F)F)c(-c3ccc(Cl)cc3O)nn2)CCO1] | 372 1.31 U | 1H NMR (400 MHz, DMSO-d6, 30° C.) δ ppm 2.80 (dd, J = 12.7, 10.0 Hz, 1 H) 2.98-3.12 (m, 1 H) 3.45- 3.57 (m, 3 H) 3.61 (td, J = 11.6, 2.6 Hz, 1 H) 3.95- 4.04 (m, 1 H) 4.25 (br d, J = 12.7 Hz, 1 H) 4.39 (br d, J = 12.7 Hz, 1 H) 4.83 (br t, J = 5.4 Hz, 1 H) 6.80 (t, J = 54.3 Hz, 1 H) 6.95-7.02 (m, 2 H) 7.29 (d, J = 7.8 Hz, 1 H) 7.40 (s, 1 H) 10.40-10.55 (m, 1 H) |
| 146 | 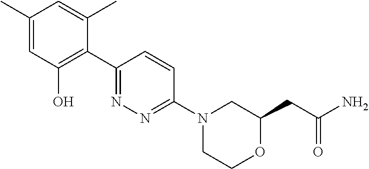 [SMILES: Cc1cc(C)c(-c2ccc(N3CCO[C@H](CC(N)=O)C3)nn2)c(O)c1] | 343.2 2.32 E | 1H NMR (500 MHz, DMSO-d6) δ (ppm): 9.38 (s, 1H), 7.40 (s, 1H), 7.36 (d, J = 9.3 Hz, 1H), 7.26 (d, J = 9.4 Hz, 1H), 6.90 (s, 1H), 6.58 (d, J = 4.4 Hz, 2H), 4.31 (d, J = 12.9 Hz, 1H), 4.10 (d, J = 12.7 Hz, 1H), 4.01-3.93 (m, 1H), 3.93-3.82 (m, 1H), 3.61 (td, J = 11.6, 2.7 Hz, 1H), 2.97 (td, J = 12.3, 3.5 Hz, 1H), 2.71 (dd, J = 12.8, 10.3 Hz, 1H), 2.42-2.26 (m, 2H), 2.23 (s, 3H), 2.03 (s, 3H). |
| 147 | 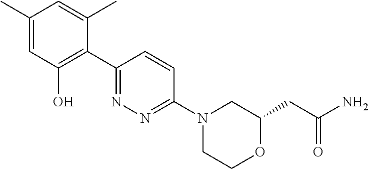 [SMILES: Cc1cc(C)c(-c2ccc(N3CCO[C@@H](CC(N)=O)C3)nn2)c(O)c1] | 343.2 2.32 E | 1H NMR (400 MHz, DMSO-d6) δ (ppm): 9.38 (s, 1H), 7.40 (s, 1H), 7.36 (d, J = 9.4 Hz, 1H), 7.26 (d, J = 9.4 Hz, 1H), 6.90 (s, 1H), 6.58 (d, J = 4.2 Hz, 2H), 4.31 (d, J = 12.7 Hz, 1H), 4.10 (d, J = 12.8 Hz, 1H), 4.01-3.93 (m, 1H), 3.87 (q, J = 7.9 Hz, 1H), 3.61 (td, J = 12.8, 3.5 Hz, 1H), 2.97 (td, J = 12.3, 3.4 Hz, 1H), 2.71 (dd, J = 12.8, 10.3 Hz, 1H), 2.42-2.25 (m, 2H), 2.23 (s, 3H), 2.04 (s, 3H). |
| 148 | 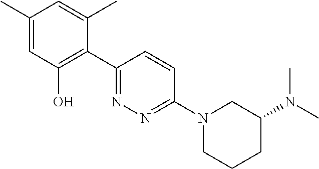 | 327 0.77 U | 1H NMR (400 MHz, DMSO-d6, 30° C.) δ ppm 1.41- 1.54 (m, 2 H) 1.75-1.84 (m, 1 H) 1.88-1.98 (m, 1 H) 2.04 (s, 3 H) 2.22 (s, 3 H) 2.22-2.26 (m, 1 H) 2.27 (s, 6 H) 2.79-2.94 (m, 2 H) 4.22 (br d, J = 12.7 Hz, 1 H) 4.46 (br d, J = 12.7 Hz, 1 H) 6.56 (br s, 1 H) 6.57 (br s, 1 H) 7.25 (d, J = 9.5 Hz, 1 H) 7.28 (d, J = 9.5 Hz, 1 H) 9.39 (br s, 1 H) |
| 149 | 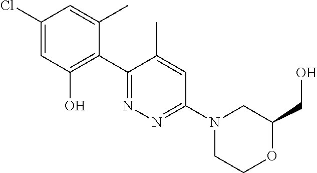 [SMILES: Cc1cc(N2CCO[C@H](CO)C2)nnc1-c1c(C)cc(Cl)cc1O] | 350 1.01 U | 1H NMR (400 MHz, DMSO-d6, 30° C.) δ ppm 1.89 (s, 3 H) 1.96 (s, 3 H) 2.70 (dd, J = 12.7, 10.0 Hz, 1 H) 2.90-3.02 (m, 1 H) 3.42-3.64 (m, 4 H) 3.95-4.02 (m, 1 H) 4.09-4,18 (m, 1 H) 4.25-4.33 (m, 1 H) 4.79 (t, J = 5.4 Hz, 1 H) 6.80 (d, J = 2.0 Hz, 1 H) 6.85 (d, J = 2.0 Hz, 1 H) 7.17 (s, 1 H) 9.81 (s, 1 H) |
| 150 | 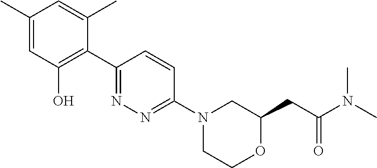 | 371.3 2.59 E | 1H NMR (500 MHz, DMSO-d6) δ (ppm): 9.38 (s, 1H), 7.36 (d, J = 9.3 Hz, 1H), 7.25 (d, J = 9.4 Hz, 1H), 6.58 (d, J = 5.4 Hz, 2H), 4.35 (d, J = 12.6 Hz, 1H), 4.13 (d, J = 12.7 Hz, 1H), 3.99-3.88 (m, 2H), 3.66-3.57 (m, 1H), 3.01 (s, 3H), 2.96 (td, J = 12.2, 3.5 Hz, 1H), 2.85 (s, 3H), 2.76-2.65 (m, 2H), 2.23 (s, 3H), 2.04 (s, 3H) + 1H hidden under DMSO peak (confirmed by HSQC and NMR in CDCl₃) |
| 151 | 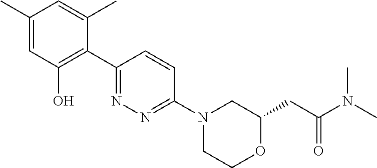 | 371.3 2.56 E | δ (ppm): 9.38 (s, 1H), 7.36 (d, J = 9.3 Hz, 1H), 7.25 (d, J = 9.4 Hz, 1H), 6.58 (d, J = 5.4 Hz, 2H), 4.35 (d, J = 12.7 Hz, 1H), 4.13 (d, J = 12.7 Hz, 1H), 3.95 (ddd, J = 17.0, 11.3, 5.4 Hz, 2H), 3.61 (td, J = 11.6, 2.7 Hz, 1H), 3.01 (s, 3H), 2.99-2.94 (m, 1H), 2.85 (s, 3H), 2.81-2.64 (m, 2H), 2.23 (s, 3H), 2.04 (s, 3H) + 1H hidden under DMSO peak (confirmed by HSQC and NMR in CDCl₃) |
| 152 | 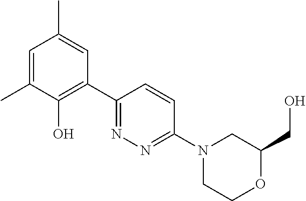 [SMILES: Cc1cc(C)c(O)c(-c2ccc(N3CCO[C@H](CO)C3)nn2)c1] | 316.2 2.98 E | 1H NMR (500 MHz, CDCl3) δ (ppm): 13.34 (s, 1H), 7.88 (d, J = 9.8 Hz, 1H), 7.25-7.23 (m, 1H), 7.09 (d, J = 9.8 Hz, 1H), 7.00 (s, 1H), 4.21-4.07 (m, 3H), 3.85-3.68 (m, 4H), 3.21-3.13 (m, 1H), 3.00 (dd, J = 12.7, 10.1 Hz, 1H), 2.31 (s, 6H), 1.93 (t, J = 5.9 Hz, 1H). |
| 153 | 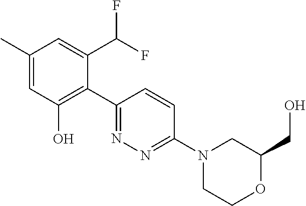 [SMILES: Cc1cc(O)c(-c2ccc(N3CCO[C@H](CO)C3)nn2)c(C(F)F)c1] | 352 1.06 U | 1H NMR (500 MHz, DMSO-d6, 25° C.) δ ppm 2.34 (s, 3 H) 2.73 (dd, J = 12.7, 10.0 Hz, 1 H) 2.99 (td, J = 12.7, 2.6 Hz, 1 H) 3.42-3.57 (m, 3 H) 3.61 (td, J = 11.6, 2.6 Hz, 1 H) 3.99 (dd, J = 11.6, 2.6 Hz, 1 H) 4.15 (br d, J = 12.7 Hz, 1 H) 4.34 (br d, J = 12.7 Hz, 1 H) 4.83 (t, J = 5.5 Hz, 1 H) 6.82 (t, J = 55.6 Hz, 1 H) 6.92 (br s, 1 H) 7.01 (br s, 1 H) 7.30 (d, J = 9.5 Hz, 1 H) 7.48 (d, J = 9.5 Hz, 1 H) 9.90 (s, 1 H) |
| 154 | 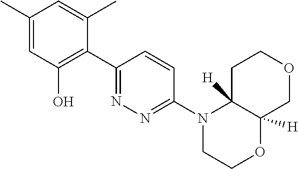 [SMILES: Cc1cc(C)c(-c2ccc(N3CCO[C@@H]4COCC[C@H]43)nn2)c(O)c1] | 342 1.21 U | 1H NMR (400 MHz, DMSO-d6, 30° C.) δ ppm 1.34- 1.46 (m, 1 H) 2.02 (s, 3 H) 2.23 (s, 3 H) 2.45-2.53 (m partially hidden, 1 H) 3.22-3.38 (m partially hidden, 2 H) 3.46-3.58 (m , 2 H) 3.66 (dt, J = 12.5, 3.0 Hz, 1 H) 3.82-3.92 (m, 3 H) 3.93-4.00 (m, 1 H) 6.59 (br s, 1 H) 6.60 (br s, 1 H) 7.39 (d, J = 9.0 Hz, 1 H) 7.45 (d, J = 9.0 Hz, 1 H) 9.39 (s, 1 H) |
| 155 | 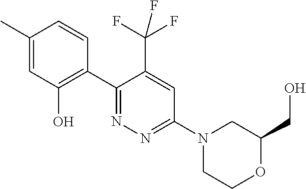 [SMILES: Cc1ccc(-c2nnc(N3CCO[C@H](CO)C3)cc2C(F)(F)F)c(O)c1] | 370 1.33 U | 1H NMR (500 MHz, DMSO-d6, 25° C.) δ ppm 2.28 (s, 3 H) 2.82 (dd, J = 12.7, 10.0 Hz, 1 H) 3.03-3.10 (m, 1 H) 3.46-3.58 (m, 3 H) 3.61 (td, J = 11.6, 2.6 Hz, 1 H) 4.00 (dd, J = 11.6, 2.6 Hz, 1 H) 4.28 (br d, J = 12.7 Hz, 1 H) 4.43 (br d, J = 12.7 Hz, 1 H) 4.83 (t, J = 5.5 Hz, 1 H) 6.68 (br d, J = 7.7 Hz, 1 H) 6.71 (br s, 1 H) 7.00 (d, J = 7.7 Hz, 1 H) 7.54 (s, 1 H) 9.37 (s, 1 H) |
| 156 | 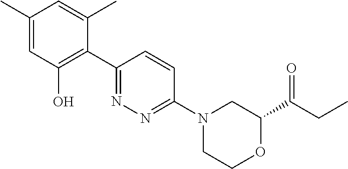 [SMILES: CCC(=O)[C@H]1CN(c2ccc(-c3c(C)cc(C)cc3O)nn2)CCO1] | 342.2 3.01 E | ¹H NMR (500 MHz, DMSO-d₆) δ (ppm): 9.37 (s, 1H), 7.35 (q, J = 9.4 Hz, 2H), 6.58 (d, J = 5.3 Hz, 2H), 4.40-4.33 (m, 1H), 4.19 (dd, J = 9.9, 3.2 Hz, 1H), 4.11-4.02 (m, 2H), 3.77-3.68 (m, 1H), 3.12 (s, 1H), 3.02 (dd, J = 13.0, 9.9 Hz, 1H), 2.67 (qd, J = 7.2, 3.0 Hz, 2H), 2.23 (s, 3H), 2.03 (s, 3H), 0.97 (t, J = 7.2 Hz, 3H). |
| 157 | 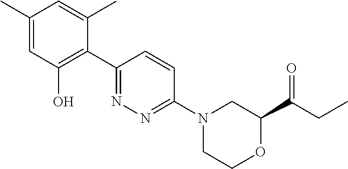 [SMILES: CCC(=O)[C@@H]1CN(c2ccc(-c3c(C)cc(C)cc3O)nn2)CCO1] | 342.2 2.99 E | ¹H NMR (400 MHz, DMSO-d₆) δ (ppm): 9.37 (s, 1H), 7.35 (q, J = 9.4 Hz, 2H), 6.58 (d, J = 5.2 Hz, 2H), 4.40-4.33 (m, 1H), 4.19 (dd, J = 10.0, 3.2 Hz, 1H), 4.13-4.02 (m, 2H), 3.77-3.68 (m, 1H), 3.12 (ddd, J = 13.7, 10.9, 3.7 Hz, 1H), 3.02 (dd, J = 13.0, 10.0 Hz, 1H), 2.68 (qt, J = 7.3, 3.7 Hz, 2H), 2.23 (s, 3H), 2.03 (s, 3H), 0.97 (t, J = 7.2 Hz, 3H). |
| 158 | 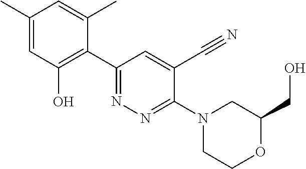 [SMILES: Cc1cc(C)c(-c2cc(C#N)c(N3CCO[C@H](CO)C3)nn2)c(O)c1] | 341 1.32 U | 1H NMR (400 MHz, DMSO-d6, 30° C.) δ ppm 2.05 (s, 3 H) 2.24 (s, 3 H) 3.01 (dd, J = 12.7, 10.0 Hz, 1 H) 3.19-3.27 (m partially hidden, 1 H) 3.42-3.73 (m, 4 H) 4.00 (br d, J = 11.6 Hz, 1 H) 4.14 (br d, J = 12.7 Hz, 1 H) 4.30 (br d, J = 12.7 Hz, 1 H) 4.83 (br t, J = 5.4 Hz, 1 H) 6.58-6.65 (m, 2 H) 7.96 (s, 1 H) 9.46 (br s, 1 H) |
| 159 | 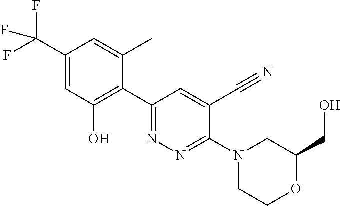 [SMILES: Cc1cc(C(F)(F)F)cc(O)c1-c1cc(C#N)c(N2CCO[C@H](CO)C2)nn1] | 395 1.46 U | 1H NMR (400 MHz, DMSO-d6, 30° C.) δ ppm 2.15 (s, 3 H) 3.05 (dd, J = 12.7, 10.0 Hz, 1 H) 3.24-3.27 (m partially hidden, 1 H) 3.42-3.49 (m, 1 H) 3.50- 3.58 (m, 1 H) 3.59-3.75 (m, 2 H) 4.01 (br d, J = 11.6 Hz, 1 H) 4.21 (br d, J = 12.7 Hz, 1 H) 4.37 (br d, J = 12.7 Hz, 1 H) 4.79-4.88 (m, 1 H) 7.09 (br s, 1 H) 7.16 (br s, 1 H) 8.07 (s, 1 H) 10.20-10.45 (m, 1 H) |
| 160 | 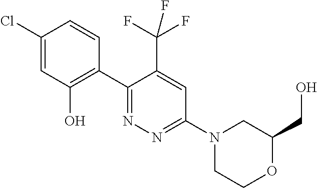 [SMILES: OC[C@@H]1CN(c2cc(C(F)(F)F)c(-c3ccc(Cl)cc3O)nn2)CCO1] | 390 1.39 U | 1H NMR (400 MHz, DMSO-d6, 30° C.) δ ppm 2.84 (dd, J = 12.7, 10.0 Hz, 1 H) 3.03-3.13 (m, 1 H) 3.45- 3.64 (m, 4 H) 4.00 (dd, J = 11.6, 2.6 Hz, 1 H) 4.29 (br d, J = 12.7 Hz, 1 H) 4.44 (br d, J = 12.7 Hz, 1 H) 4.84 (br t, J = 5.4 Hz, 1 H) 6.91-6.96 (m, 2 H) 7.17 (d, J = 8.5 Hz, 1 H) 7.57 (s, 1 H) 10.09 (br s, 1 H) |
| 161 | 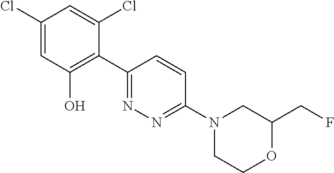 [SMILES: Oc1cc(Cl)cc(Cl)c1-c1ccc(N2CCOC(CF)C2)nn1] | 358 1.50 U | 1H NMR (400 MHz, DMSO-d6, 30° C.) δ ppm 2.85 (dd, J = 12.7, 10.8 Hz, 1 H) 2.97-3.07 (m, 1 H) 3.65 (td, J = 11.6, 2.6 Hz, 1 H) 3.73-3.88 (m, 1 H) 4.00- 4.07 (m, 1 H) 4.16 (br d, J = 12.7 Hz, 1 H) 4.34 (br d, J = 12.7 Hz, 1 H) 4.45-4.65 (m, 2 H) 6.93 (d, J = 2.0 Hz, 1 H) 7.08 (d, J = 2.0 Hz, 1 H) 7.32 (d, J = 9.5 Hz, 1 H) 7.41 (d, J = 9.5 Hz, 1 H) 10.30-10.80 (m, 1 H) |
| 162 | 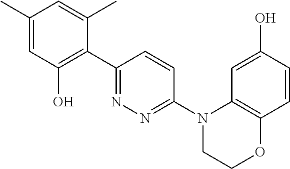 [SMILES: Cc1cc(C)c(-c2ccc(N3CCOc4ccc(O)cc43)nn2)c(O)c1] | 350 1.50 U | 1H NMR (400 MHz, DMSO-d6, 30° C.) δ ppm 2.01 (s, 3 H) 2.23 (s, 3 H) 3.26-3.33 (m, 2 H) 4.08-4.16 (m, 2 H) 6.31 (dd, J = 8.5, 2.8 Hz, 1 H) 6.43 (d, J = 2.8 Hz, 1 H) 6.59 (br s, 1 H) 6.61 (br s, 1 H) 6.69 (d, J = 8.5 Hz, 1 H) 7.29 (d, J = 9.5 Hz, 1 H) 7.62 (d, J = 9.5 Hz, 1 H) 9.30-9.50 (br s, 1 H) |
| 163 | 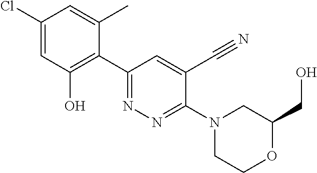 [SMILES: Cc1cc(Cl)cc(O)c1-c1cc(C#N)c(N2CCO[C@H](CO)C2)nn1] | 361 1.39 U | 1H NMR (400 MHz, DMSO-d6, 30° C.) δ ppm 2.07 (s, 3 H) 3.03 (dd, J = 12.7, 10.0 Hz, 1 H) 3.22-3.28 (m partially hidden, 1 H) 3.41-3.48 (m, 1 H) 3.51- 3.57 (m, 1 H) 3.60-3.73 (m, 2 H) 3.97-4.04 (m, 1 H) 4.15-4.21 (m, 1 H) 4.29-4.38 (m, 1 H) 4.83 (t, J = 5.4 Hz, 1 H) 6.84 (d, J = 2.0 Hz, 1 H) 6.88 (d, J = 2.0 Hz, 1 H) 8.01 (s, 1 H) 10.12 (br s, 1 H) |
| 164 | 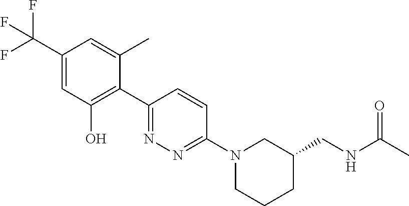 [SMILES: CC(=O)NC[C@@H]1CCCN(c2ccc(-c3c(C)cc(C(F)(F)F)cc3O)nn2)C1] | 409 1.16 U | 1H NMR (400 MHz, DMSO-d6, 30° C.) δ ppm 1.17- 1.30 (m, 1 H) 1.42-1.55 (m, 1 H) 1.61-1.85 (m, 3 H) 1.84 (s, 3 H) 2.13 (s, 3 H) 2.64-2.76 (m, 1 H) 2.94-3.09 (m, 3 H) 4.23 (br d, J = 12.7 Hz, 1 H) 4.33 (br dd, J = 12.7, 2.6 Hz, 1 H) 7.06 (br s, 1 H) 7.11 (br s, 1 H) 7.28 (d, J = 9.5 Hz, 1 H) 7.35 (d, J = 9.5 Hz, 1 H) 7.90 (br t, J = 5.8 Hz, 1 H) 10.13 (br s, 1 H) |
| 165 | 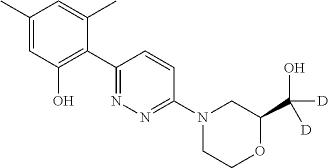 [SMILES: [2H]C([2H])(O)[C@@H]1CN(c2ccc(-c3c(C)cc(C)cc3O)nn2)CCO1] | 318.4 2.26 E | ¹H NMR (400 MHz, DMSO-d₆) δ (ppm): 9.38 (s, 1H), 7.35 (d, J = 9.4 Hz, 1H), 7.26 (d, J = 9.5 Hz, 1H), 6.63-6.46 (m, 2H), 4.78 (s, 1H), 4.34-4.27 (m, 1H), 4.20-4.05 (m, 1H), 4.08-3.90 (m, 1H), 3.61 (td, J = 11.6, 2.8 Hz, 1H), 3.53 (dd, J = 10.6, 2.6 Hz, 1H), 3.18-2.81 (m, 1H), 2.70 (dd, J = 12.8, 10.6 Hz, 1H), 2.22 (s, 3H), 2.03 (s, 3H). |
| 166 | 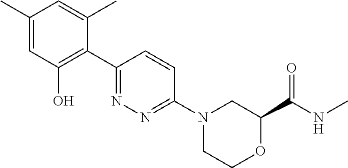 | 343.3 2.41 E | ¹H NMR (400 MHz, DMSO-d₆) δ (ppm): 9.39 (s, 1H), 7.89 (d, J = 4.9 Hz, 1H), 7.46 - 7.11 (m, 2H), 6.58 (d, J = 4.6 Hz, 2H), 4.43 (dd, J = 12.2, 2.6 Hz, 1H), 4.18 - 3.93 (m, 3H), 3.71 (td, J = 11.1, 2.5 Hz, 1H), 3.16 - 3.02 (m, 1H), 2.94 (dd, J = 13.0, 10.2 Hz, 1H), 2.64 (d, J = 4.7 Hz, 3H), 2.22 (s, 3H), 2.03 (s, 3H). |
| 167 | 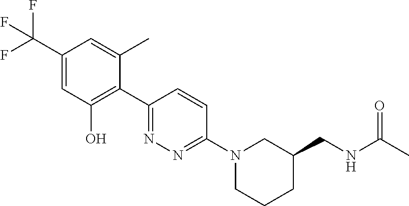 [SMILES: CC(=O)NC[C@H]1CCCN(c2ccc(-c3c(C)cc(C(F)(F)F)cc3O)nn2)C1] | 409 1.15 U | 1H NMR (400 MHz, DMSO-d6, 30° C.) δ ppm 1.18- 1.31 (m, 2 H) 1.42-1.55 (m, 1 H) 1.66-1.85 (m, 2 H) 1.84 (s, 3 H) 2.13 (s, 3 H) 2.70 (dd, J = 12.7, 10.0 Hz, 1 H) 2.93-3.09 (m, 3 H) 4.23 (br d, J = 12.7 Hz, 1 H) 4.30-4.37 (m, 1 H) 7.06 (br s, 1 H) 7.11 (br s, 1 H) 7.29 (d, J = 9.5 Hz, 1 H) 7.36 (d, J = 9.5 Hz, 1 H) 7.92 (br t, J = 5.8 Hz, 1 H) 10.15 (br s, 1 H) |
| 168 | 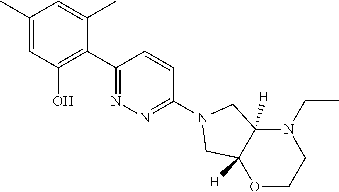 [SMILES: CCN1CCO[C@@H]2CN(c3ccc(-c4c(C)cc(C)cc4O)nn3)C[C@H]21] | 355.5 2.54 E | 1H NMR (400 MHz, DMSO-d6) δ (ppm): 9.36 (s, 1H), 7.30 (d, J = 9.3 Hz, 1H), 6.91 (d, J = 9.3 Hz, 1H), 6.67-6.41 (m, 2H), 4.05-3.91 (m, 2H), 3.92- 3.80 (m, 1H), 3.77-3.61 (m, 2H), 3.25 (t, J = 9.5 Hz, 1H), 3.13 (t, J = 10.0 Hz, 1H), 2.97-2.85 (m, 1H), 2.74-2.59 (m, 1H), 2.41-2.35 (m, 1H), 2.29- 2.23 (m, 1H), 2.22 (s, 3H), 2.17-2.06 (m, 1H), 2.02 (s, 3H), 1.05 (t, J = 7.2 Hz, 3H). |
| 169 | 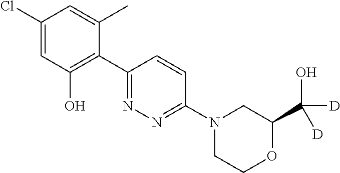 [SMILES: [2H]C([2H])(O)[C@@H]1CN(c2ccc(-c3c(C)cc(Cl)cc3O)nn2)CCO1] | 338.4 3.38 E | 1H NMR (400 MHz, DMSO-d6) δ (ppm): 9.97 (s, 1H), 7.37 (d, J = 9.4 Hz, 1H), 7.28 (d, J = 9.5 Hz, 1H), 6.91-6.72 (m, 2H), 4.78 (s, 1H), 4.45-4.21 (m, 1H), 4.17-4.06 (m, 1H), 4.07-3.88 (m, 1H), 3.71-3.56 (m, 1H), 3.52 (dd, J = 10.6, 2.6 Hz, 1H), 3.08-2.88 (m, 1H), 2.78-2.64 (m, 1H), 2.05 (s, 3H). |
| 170 | 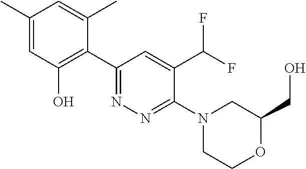 [SMILES: Cc1cc(C)c(-c2cc(C(F)F)c(N3CCO[C@H](CO)C3)nn2)c(O)c1] | 366 1.40 U | 1H NMR (500 MHz, DMSO-d6, 27° C.) δ ppm 2.04 (s, 3 H) 2.24 (s, 3 H) 2.94 (dd, J = 12.7, 10.0 Hz, 1 H) 3.12-3.18 (m, 1 H) 3.41-3.54 (m, 3 H) 3.62 (dt, J = 12.7, 2.3 Hz, 1 H) 3.64-3.71 (m, 1 H) 3.76 (td, J = 11.6, 2.5 Hz, 1 H) 3.93-3.96 (m, 1 H) 4.77 (t, J = 5.4 Hz, 1 H) 6.62 (s, 2 H) 7.22 (t, J = 53.6 Hz, 1 H) 7.67 (s, 1 H) 9.47 (s, 1 H) |
| 171 | 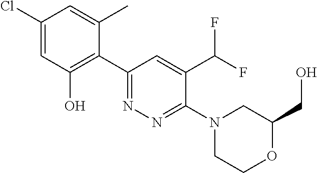 [SMILES: Cc1cc(Cl)cc(O)c1-c1cc(C(F)F)c(N2CCO[C@H](CO)C2)nn1] | 386 1.46 U | 1H NMR (400 MHz, DMSO-d6, 30° C.) 8 ppm 2.06 (s, 3 H) 2.95 (dd, J = 12.7, 10.0 Hz, 1 H) 3.11-3.22 (m, 1 H) 3.41-3.55 (m, 3 H) 3.63-3.79 (m, 3 H) 3.92-3.98 (m, 1 H) 4.76 (t, J = 5.4 Hz, 1 H) 6.85 (d, J = 2.0 Hz, 1 H) 6.89 (d, J = 2.0 Hz, 1 H) 7.22 (t, J = 53.6 Hz, 1 H) 7.71 (s, 1 H) 10.09 (s, 1 H) |
| 172 | 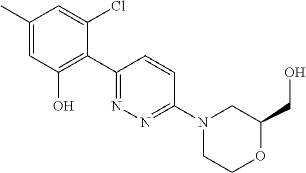 [SMILES: Cc1cc(O)c(-c2ccc(N3CCO[C@H](CO)C3)nn2)c(Cl)c1] | 336 1.13 U | 1H NMR (400 MHz, DMSO-d6, 30° C.) δ ppm 2.27 (s, 3 H) 2.73 (dd, J = 12.7, 10.0 Hz, 1 H) 2.99 (td, J = 12.7, 2.6 Hz, 1 H) 3.44-3.65 (m, 4 H) 3.99 (dd, J = 11.6, 2.6 Hz, 1 H) 4.14 (br d, J = 12.7 Hz, 1 H) 4.33 (br d, J = 12.7 Hz, 1 H) 4.79-4.88 (m, 1 H) 6.73 (s, 1 H) 6.83 (s, 1 H) 7.28 (d, J = 9.5 Hz, 1 H) 7.37 (d, J = 9.5 Hz, 1 H) 9.94 (br s, 1 H) |
| 173 | 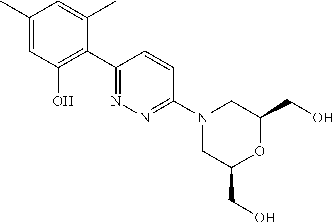 [SMILES: Cc1cc(C)c(-c2ccc(N3C[C@@H](CO)O[C@@H](CO)C3)nn2)c(O)c1] | 346 0.92 U | 1H NMR (400 MHz, DMSO-d6, 30° C.) δ ppm 2.03 (s, 3 H) 2.22 (s, 3 H) 2.60-2.69 (m, 2 H) 3.44-3.63 (m, 6 H) 4.33 (br d, J = 12.7 Hz, 2 H) 4.77 (br t, J = 5.5 Hz, 2 H) 6.57 (br s, 1 H) 6.58 (br s, 1 H) 7.24 (d, J = 9.5 Hz, 1 H) 7.35 (d, J = 9.5 Hz, 1 H) 9.37 (br s, 1 H) |
| 174 | 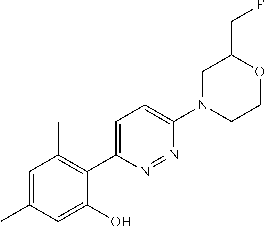 [SMILES: Cc1cc(C)c(-c2ccc(N3CCOC(CF)C3)nn2)c(O)c1] | 318 1.10 U | 1H NMR (400 MHz, DMSO-d6, 30° C.) δ ppm 2.03 (s, 3 H) 2.22 (s, 3 H) 2.82 (dd, J = 12.7, 10.8 Hz, 1 H) 2.93-3.04 (m, 1 H) 3.66 (td, J = 11.6, 2.6 Hz, 1 H) 3.75-3.87 (m, 1 H) 4.00-4.06 (m, 1 H) 4.13 (br d, J = 12.7 Hz, 1 H) 4.31 (br d, J = 11.5 Hz, 1 H) 4.45- 4.65 (m, 2 H) 6.57 (br s, 1 H) 6.58 (br s, 1 H) 7.29 (d, J = 9.5 Hz, 1 H) 7.36 (d, J = 9.5 Hz, 1 H) 9.37 (br s, 1 H) |
| 175 | 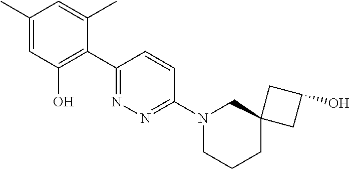 [SMILES: Cc1cc(C)c(-c2ccc(N3CCC[C@]4(C3)C[C@H](O)C4)nn2)c(O)c1] | 340 1.01 U | 1H NMR (400 MHz, DMSO-d6, 30° C.) δ ppm 1.49- 1.63 (m, 6 H) 2.04 (s, 3 H) 2.09-2.17 (m, 2 H) 2.22 (s, 3 H) 3.49 (s, 2 H) 3.53 (br t, J = 5.1 Hz, 2 H) 4.18- 4.30 (m, 1 H) 4.94 (d, J = 6.3 Hz, 1 H) 6.56 (br s, 1 H) 6.58 (br s, 1 H) 7.26 (d, J = 9.5 Hz, 1 H) 7.30 (d, J = 9.5 Hz, 1 H) 9.41 (br s, 1 H) |
| 176 | 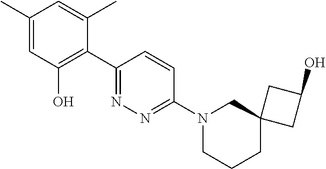 [SMILES: Cc1cc(C)c(-c2ccc(N3CCC[C@]4(C3)C[C@@H](O)C4)nn2)c(O)c1] | 340 1.02 U | 1H NMR (400 MHz, DMSO-d6, 30° C.) δ ppm 1.57- 1.62 (m, 4 H) 1.63-1.70 (m, 2 H) 2.04 (s, 3 H) 2.04- 2.10 (m, 2 H) 2.22 (s, 3 H) 3.49-3.53 (m, 2 H) 3.53 (s, 2 H) 4.06-4.17 (m, 1 H) 4.94 (d, J = 6.3 Hz, 1 H) 6.56 (br s, 1 H) 6.57 (br s, 1 H) 7.25 (d, J = 9.5 Hz, 1 H) 7.28 (d, J = 9.5 Hz, 1 H) 9.42 (br s, 1 H) |
| 177 | 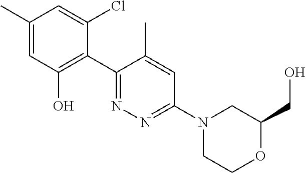 [SMILES: Cc1cc(O)c(-c2nnc(N3CCO[C@H](CO)C3)cc2C)c(Cl)c1] | 350 1.06 U | 1H NMR (400 MHz, DMSO-d6, 30° C.) δ ppm 1.96 (s, 3 H) 2.28 (s, 3 H) 2.70 (dd, J = 12.7, 10.0 Hz, 1 H) 2.96 (td, J = 12.7, 2.6 Hz, 1 H) 3.36-3.64 (m, 4 H) 3.99 (dd, J = 11.6, 2.6 Hz, 1 H) 4.10-4.18 (m, 1 H) 4.25-4.35 (m, 1 H) 4.82 (br t, J = 5.4 Hz, 1 H) 6.72 (br s, 1 H) 6.84 (br s, 1 H) 7.17 (br s, 1 H) 9.77 (br s, 1 H) |
| 178 | 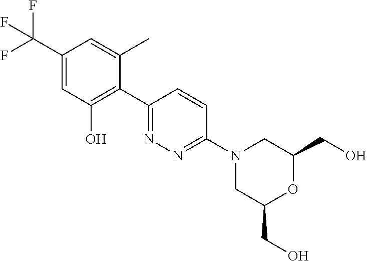 [SMILES: Cc1cc(C(F)(F)F)cc(O)c1-c1ccc(N2C[C@@H](CO)O[C@@H](CO)C2)nn1] | 400 1.18 U | 1H NMR (400 MHz, DMSO-d6, 30° C.) δ ppm 2.13 (s, 3 H) 2.67 (dd, J = 12.7, 10.6 Hz, 2 H) 3.42-3.63 (m, 6 H) 4.32-4.40 (m, 2 H) 4.77-4.85 (m, 2 H) 7.07 (br s, 1 H) 7.12 (br s, 1 H) 7.30 (d, J = 9.5 Hz, 1 H) 7.41 (d, J = 9.5 Hz, 1 H) 10.10-10.35 (m, 1 H) |
| 179 | 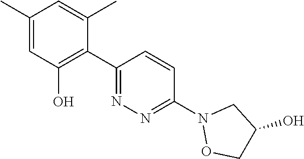 [SMILES: Cc1cc(C)c(-c2ccc(N3C[C@H](O)CO3)nn2)c(O)c1] | 288 1.06 U | 1H NMR (400 MHz, DMSO-d6, 30° C.) δ ppm 2.01 (s, 3 H) 2.23 (s, 3 H) 3.78-3.85 (m, 2 H) 3.90 (dd, J = 9.0, 5.0 Hz, 1 H) 4.11 (dd, J = 11.0, 6.0 Hz, 1 H) 4.71-4.76 (m, 1 H) 5.35 (br d, J = 4.0 Hz, 1 H) 6.58 (br s, 1 H) 6.60 (br s, 1 H) 7.41 (d, J = 9.0 Hz, 1 H) 7.48 (d, J = 9.0 Hz, 1 H) 9.35 (br s, 1 H) |
| 180 | 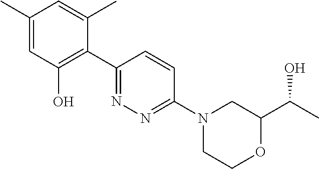 [SMILES: Cc1cc(C)c(-c2ccc(N3CCOC([C@@H](C)O)C3)nn2)c(O)c1] | 330.4 2.41 E | 1H NMR (400 MHz, DMSO) δ (ppm): 9.40 (s, 1H), 7.35 (d, J = 9.4 Hz, 1H), 7.26 (d, J = 9.4 Hz, 1H), 6.61-6.55 (m, 2H), 4.85 (d, J = 5.5 Hz, 1H), 4.52- 4.39 (m, 1H), 4.16-4.06 (m, 1H), 4.04-3.96 (m, 1H), 3.67-3.54 (m, 2H), 3.27-3.18 (m, 1H), 3.01- 2.92 (m, 1H), 2.72 (dd, J = 13.0, 10.4 Hz, 1H), 2.23 (s, 3H), 2.04 (s, 3H), 1.17 (d, J = 6.3 Hz, 3H). |
| 181 | 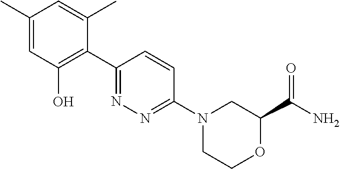 [SMILES: Cc1cc(C)c(-c2ccc(N3CCO[C@H](C(N)=O)C3)nn2)c(O)c1] | 329.4 2.22 E | 1H NMR (400 MHz, DMSO-d6) δ (ppm): 9.45 (s, 1H), 7.47-7.24 (m, 4H), 6.67-6.51 (m, 2H), 4.48- 4.34 (m, 1H), 4.16-3.95 (m, 3H), 3.78-3.66 (m, 1H), 3.15-3.05 (m, 1H), 2.96 (dd, J = 13.0, 10.2 Hz, 1H), 2.23 (s, 3H), 2.03 (s, 3H). |
| 182 | 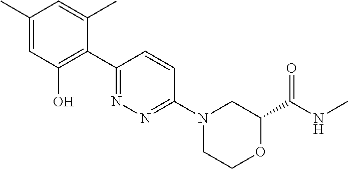 | 343.2 1.24 P | ¹H NMR (500 MHz, DMSO-d₆) δ (ppm): 9.39 (s, 1H), 7.88 (d, J = 5.1 Hz, 1H), 7.36 (d, J = 9.4 Hz, 1H), 7.30 (d, J = 9.3 Hz, 1H), 6.59 (s, 1H), 6.57 (s, 1H), 4.43 (d, J = 12.6 Hz, 1H), 4.13-4.03 (m, 3H), 3.75-3.67 (m, 1H), 3.09 (t, J = 11.0 Hz, 1H), 2.94 (dd, J = 13.0, 10.2 Hz, 1H), 2.64 (d, J = 4.7 Hz, 3H), 2.22 (s, 3H), 2.03 (s, 3H). |
| 183 | 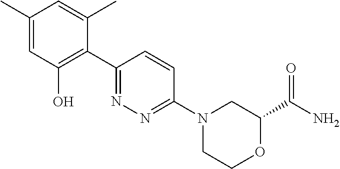 [SMILES: Cc1cc(C)c(-c2ccc(N3CCO[C@@H](C(N)=O)C3)nn2)c(O)c1] | 329.2 2.33 E | ¹H NMR (500 MHz, DMSO-d₆) δ (ppm): 9.39 (s, 1H), 7.40-7.28 (m, 4H), 6.58 (s, 1H), 6.57 (s, 1H), 4.40 (d, J = 13.1 Hz, 1H), 4.11-3.99 (m, 3H), 3.74- 3.66 (m, 1H), 3.15-3.06 (m, 1H), 2.96 (dd, J = 13.0, 10.2 Hz, 1H), 2.22 (s, 3H), 2.03 (s, 3H). |
| 184 | 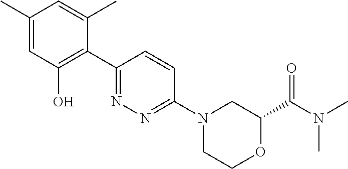 | 357.2 1.22 P | ¹H NMR (500 MHz, DMSO-d₆) δ (ppm): 9.39 (s, 1H), 7.35 (d, J = 9.4 Hz, 1H), 7.31 (d, J = 9.4 Hz, 1H), 6.58 (s, 1H), 6.57 (s, 1H), 4.39 (dd, J = 10.0, 2.8 Hz, 1H), 4.24 (d, J = 13.2 Hz, 1H), 4.17 (d, J = 13.1 Hz, 1H), 4.00 (d, J = 12.0 Hz, 1H), 3.73 (td, J = 11.4, 2.8 Hz, 1H), 3.19-3.02 (m, 2H), 3.07 (s, 3H), 2.86 (s, 3H), 2.22 (s, 3H), 2.03 (s, 3H). |
| 185 | 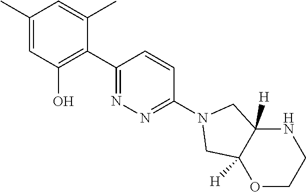 [SMILES: Cc1cc(C)c(-c2ccc(N3C[C@@H]4NCCO[C@H]4C3)nn2)c(O)c1] | 327.2 2.37 E | ¹H NMR (400 MHz, CDCl₃) δ (ppm): 7.50 (d, J = 9.5 Hz, 1H), 6.81-6.71 (m, 2H), 6.68-6.59 (m, 1H), 4.07-3.86 (m, 3H), 3.81 (td, J = 11.7, 2.9 Hz, 1H), 3.76-3.67 (m, 1H), 3.40 (t, J = 9.5 Hz, 1H), 3.30 (t, J = 10.1 Hz, 1H), 3.18-3.05 (m, 2H), 3.05- 2.97 (m, 1H), 2.37 (s, 3H), 2.30 (s, 3H). |
| 186 | 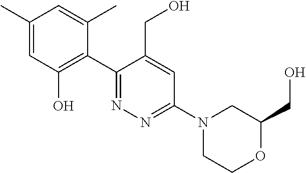 [SMILES: Cc1cc(C)c(-c2nnc(N3CCO[C@H](CO)C3)cc2CO)c(O)c1] | 346 0.84 U | 1H NMR (500 MHz, DMSO-d6, 27° C.) δ ppm 1.84 (s, 3 H) 2.23 (s, 3 H) 2.72 (dd, J = 12.7, 10.0 Hz, 1 H) 2.94-3.01 (m, 1 H) 3.44-3.65 (m, 4 H) 3.95-4.02 (m, 2 H) 4.12-4.18 (m, 1 H) 4.24-4.30 (m, 2 H) 4.84 (t, J = 5.4 Hz, 1 H) 5.34 (br t, J = 5.4 Hz, 1 H) 6.56 (br s, 1 H) 6.58 (br s, 1 H) 7.21 (br s, 1 H) 9.20 (s, 1 H) |
| 187 | 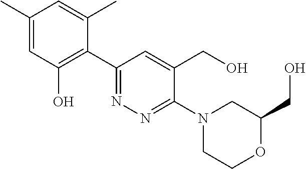 [SMILES: Cc1cc(C)c(-c2cc(CO)c(N3CCO[C@H](CO)C3)nn2)c(O)c1] | 346 0.97 U | 1H NMR (500 MHz, DMSO-d6, 27° C.) δ ppm 2.01 (s, 3 H) 2.24 (s, 3 H) 2.79 (dd, J = 12.7, 10.0 Hz, 1 H) 2.98-3.04 (m, 1 H) 3.35-3.53 (m, 4 H) 3.60-3.66 (m, 1 H) 3.71 (td, J = 11.6, 2.6 Hz, 1 H) 3.90-3.94 (m, 1 H) 4.53 (d, J = 5.4 Hz, 2 H) 4.75 (t, J = 5.4 Hz, 1 H) 5.55 (t, J = 5.4 Hz, 1 H) 6.59 (br s, 1 H) 6.60 (br s, 1 H) 7.53 (s, 1 H) 9.39 (s, 1 H) |
| 188 | 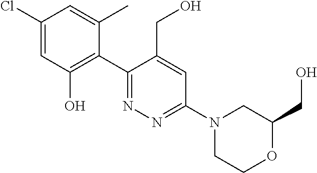 [SMILES: Cc1cc(Cl)cc(O)c1-c1nnc(N2CCO[C@H](CO)C2)cc1CO] | 366 0.92 U | 1H NMR (500 MHz, DMSO-d6, 27° C.) δ ppm 1.88 (s, 3 H) 2.73 (dd, J = 12.7, 10.0 Hz, 1 H) 2.95-3.02 (m, 1 H) 3.44-3.65 (m, 4 H) 3.97-4.02 (m, 2 H) 4.13-4.19 (m, 1 H) 4.23-4.31 (m, 2 H) 4.84 (t, J = 5.4 Hz, 1 H) 5.30-5.55 (m, 1 H) 6.79 (d, J = 2.0 Hz, 1 H) 6.85 (d, J = 2.0 Hz, 1 H) 7.23 (s, 1 H) 9.75- 10.0 (m, 1 H) |
| 189 | 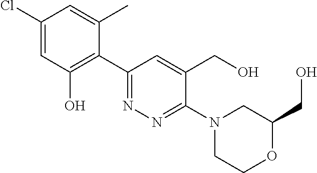 [SMILES: Cc1cc(Cl)cc(O)c1-c1cc(CO)c(N2CCO[C@H](CO)C2)nn1] | 366 1.10 U | 1H NMR (500 MHz, DMSO-d6, 27° C.) δ ppm 2.03 (s, 3 H) 2.80 (dd, J = 12.7, 10.2 Hz, 1 H) 2.97-3.05 (m, 1 H) 3.36-3.52 (m, 4 H) 3.60-3.66 (m, 1 H) 3.68-3.74 (m, 1 H) 3.89-3.96 (m, 1 H) 4.53 (br d, J = 5.4 Hz, 2 H) 4.76 (br t, J = 5.4 Hz, 1 H) 5.57 (t, J = 5.4 Hz, 1 H) 6.84 (d, J = 2.0 Hz, 1 H) 6.86 (d, J = 2.0 Hz, 1 H) 7.54 (s, 1 H) 9.90-10.10 (m, 1 H) |
| 190 | 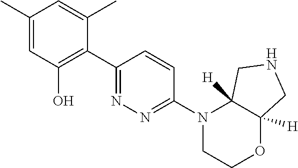 [SMILES: Cc1cc(C)c(-c2ccc(N3CCO[C@@H]4CNC[C@H]43)nn2)c(O)c1] | 327.3 2.20 E | ¹H NMR (500 MHz, DMSO-d₆) δ (ppm): 9.37 (br. s, 1H), 7.38 (d, J = 9.3 Hz, 1H), 7.32 (d, J = 9.3 Hz, 1H), 6.57 (d, J = 6.8 Hz, 2H), 4.13-4.04 (m, 1H), 3.94-3.75 (m, 3H), 3.70-3.60 (m, 1H), 3.13- 3.01 (m, 1H), 3.01-2.90 (m, 2H), 2.81-2.69 (m, 2H), 2.22 (s, 3H), 2.00 (s, 3H). (NH not visible) |
| 191 | 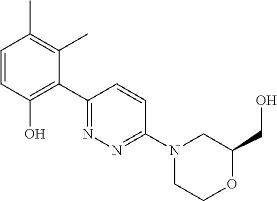 [SMILES: Cc1ccc(O)c(-c2ccc(N3CCO[C@H](CO)C3)nn2)c1C] | 316.2 2.33 E | ¹H NMR (400 MHz, DMSO) δ (ppm): 10.97 (s, 1H), 7.55 (d, J = 9.6 Hz, 1H), 7.11-7.01 (m, 2H), 6.75 (d, J = 7.6 Hz, 1H), 4.24 (d, J = 12.8 Hz, 1H), 4.16 (ddt, J = 11.5, 3.4, 2.0 Hz, 2H), 3.89-3.68 (m, 4H), 3.27-3.15 (m, 1H), 3.04 (dd, J = 12.8, 9.9 Hz, 1H), 2.39 (s, 3H), 2.31 (s, 3H), 2.00 (s, 1H). |
| 192 | 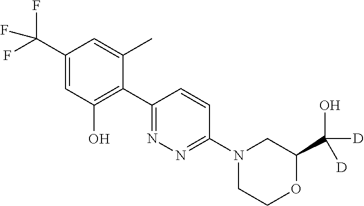 [SMILES: [2H]C([2H])(O)[C@@H]1CN(c2ccc(-c3c(C)cc(C(F)(F)F)cc3O)nn2)CCO1] | 372.2 2.68 E | ¹H NMR (500 MHz, DMSO-d₆) δ (ppm): 10.15 (s, 1H), 7.41 (d, J = 9.4 Hz, 1H), 7.31 (d, J = 9.5 Hz, 1H), 7.12 (s, 1H), 7.06 (s, 1H), 4.79 (s, 1H), 4.37- 4.28 (m, 1H), 4.21-4.10 (m, 1H), 4.06-3.93 (m, 1H), 3.67-3.56 (m, 1H), 3.53 (dd, J = 10.4, 2.6 Hz, 1H), 3.07-2.92 (m, 1H), 2.82-2.64 (m, 1H), 2.13 (s, 3H). |
| 193 | 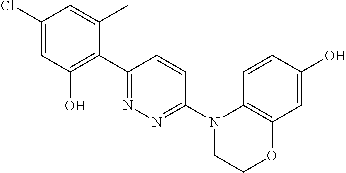 [SMILES: Cc1cc(Cl)cc(O)c1-c1ccc(N2CCOc3cc(O)ccc32)nn1] | 370 1.53 U | 1H NMR (400 MHz, DMSO-d6, 30° C.) δ ppm 2.03 (s, 3 H) 3.26-3.33 (m hidden, 2 H) 4.12-4.18 (m, 2 H) 6.56-6.63 (m, 3 H) 6.83 (d, J = 2.0 Hz, 1 H) 6.85 (d, J = 2.0 Hz, 1 H) 7.30 (d, J = 9.5 Hz, 1 H) 7.63 (d, J = 9.5 Hz, 1 H) 9.95-10.15 (m, 1 H) |
| 194 | 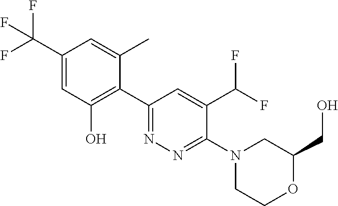 [SMILES: Cc1cc(C(F)(F)F)cc(O)c1-c1cc(C(F)F)c(N2CCO[C@H](CO)C2)nn1] | 420 1.53 U | 1H NMR (400 MHz, DMSO-d6, 30° C.) δ ppm 2.14 (s, 3 H) 2.96 (dd, J = 12.7, 10.0 Hz, 1 H) 3.10-3.25 (m, 1 H) 3.37-3.56 (m, 3 H) 3.64-3.72 (m, 2 H) 3.76 (td, J = 11.6, 2.6 Hz, 1 H) 3.92-3.98 (m, 1 H) 4.79 (t, J = 5.4 Hz, 1 H) 7.10 (br s, 1 H) 7.17 (br s, 1 H) 7.24 (t, J = 53.3 Hz, 1 H) 7.78 (s, 1 H) 10.40- 10.70 (m, 1 H) |
| 195 | 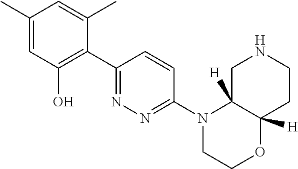 [SMILES: Cc1cc(C)c(-c2ccc(N3CCO[C@H]4CCNC[C@H]43)nn2)c(O)c1] | 341 0.84 U | 1H NMR (400 MHz, DMSO-d6, 30° C.) δ ppm 1.71- 1.81 (m, 2 H) 2.03 (s, 3 H) 2.04-2.21 (m, 1 H) 2.22 (s, 3 H) 2.58-2.69 (m, 3 H) 2.91 (t, J = 11.8 Hz, 1 H) 3.14 (td, J = 11.8, 3.8 Hz, 1 H) 3.61 (td, J = 11.8, 3.8 Hz, 1 H) 3.79-3.87 (m, 1 H) 3.97 (br d, J = 11.8 Hz, 1 H) 4.06 (dd, J = 11.8, 3.8 Hz, 1 H) 4.15-4.26 (m, 1 H) 6.57 (br s, 1 H) 6.58 (br s, 1 H) 7.19 (d, J = 9.5 Hz, 1 H) 7.33 (d, J = 9.5 Hz, 1 H) 9.39 (br s, 1 H) |
| 196 | 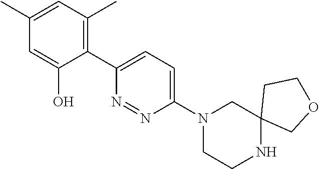 [SMILES: Cc1cc(C)c(-c2ccc(N3CCNC4(CCOC4)C3)nn2)c(O)c1] | 341.3 2.38 E | 1H NMR (500 MHz, DMSO-d6) δ (ppm): 9.38 (br. s, 1H), 7.29 (d, J = 6.3 Hz, 2H), 6.57 (br. s, 2H), 3.82 (br. s, 2H), 3.63 (d, J = 8.6 Hz, 1H), 3.57-3.44 (m, 5H), 2.85 (d, J = 22.9 Hz, 2H), 2.22 (br. s, 3H), 2.03 (br. s, 3H), 1.86 (br. s, 1H), 1.80 (br. s, 1H). Another proton hidden under DMSO peak. |
| 197 | 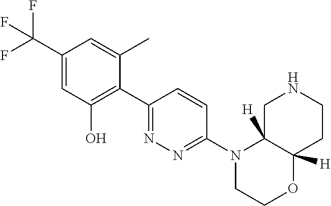 [SMILES: Cc1cc(C(F)(F)F)cc(O)c1-c1ccc(N2CCO[C@H]3CCNC[C@H]32)nn1] | 365 1.01 U | 1H NMR (400 MHz, DMSO-d6, 30° C.) δ ppm 1.71- 1.80 (m, 2 H) 2.13 (s, 3 H) 2.58-2.66 (m, 3 H) 2.92 (t, J = 11.8 Hz, 1 H) 3.16 (td, J = 11.8, 3.8 Hz, 1 H) 3.62 (td, J = 11.8, 3.8 Hz, 1 H) 3.78-3.89 (m, 1 H) 3.99 (br d, J = 11.8 Hz, 1 H) 4.06 (dd, J = 11.8, 3.8 Hz, 1 H) 4.17 - 4.30 (m, 1 H) 7.06 (br s, 1 H) 7.11 (br s, 1 H) 7.24 (d, J = 9.5 Hz, 1 H) 7.39 (d, J = 9.5 Hz, 1 H) 9.02-11.04 (m, 1 H) |
| 198 | 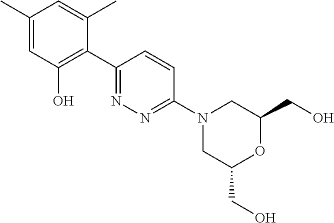 [SMILES: Cc1cc(C)c(-c2ccc(N3C[C@@H](CO)O[C@H](CO)C3)nn2)c(O)c1] | 346 0.90 U | 1H NMR (500 MHz, DMSO-d6, 27° C.) δ ppm 2.03 (s, 3 H) 2.22 (s, 3 H) 3.39-3.48 (m, 2 H) 3.49-3.59 (m, 4 H) 3.77 (dd, J = 12.7, 3.7 Hz, 2 H) 3.81-3.89 (m, 2 H) 4.76 (t, J = 5.7 Hz, 2 H) 6.57 (br s, 1 H) 6.58 (br s, 1 H) 7.22 (d, J = 9.5 Hz, 1 H) 7.33 (d, J = 9.5 Hz, 1 H) 9.40 (br s, 1 H) |
| 199 | 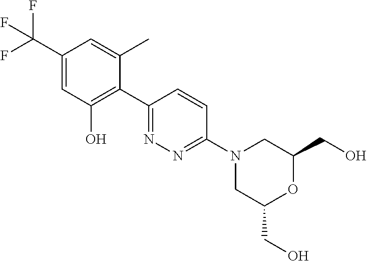 [SMILES: Cc1cc(C(F)(F)F)cc(O)c1-c1ccc(N2C[C@@H](CO)O[C@H](CO)C2)nn1] | 400 1.15 U | 1H NMR (400 MHz, DMSO-d6, 30° C.) δ ppm 2.13 (s, 3 H) 3.46 (br dd, J = 12.7, 6.5 Hz, 2 H) 3.50-3.58 (m, 4 H) 3.75-3.90 (m, 4 H) 4.69-4.85 (m, 2 H) 7.07 (br s, 1 H) 7.10 (br s, 1 H) 7.26 (d, J = 9.5 Hz, 1 H) 7.40 (d, J = 9.5 Hz, 1 H) 10.05-10.80 (m, 1 H) |
| 200 | 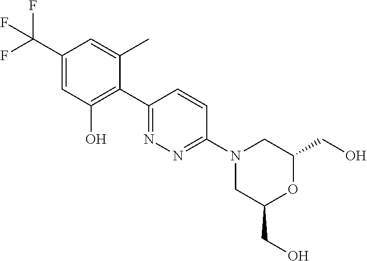 [SMILES: Cc1cc(C(F)(F)F)cc(O)c1-c1ccc(N2C[C@H](CO)O[C@@H](CO)C2)nn1] | 400 1.15 U | 1H NMR (400 MHz, DMSO-d6, 30° C.) δ ppm 2.13 (s, 3 H) 3.46 (dd, J = 12.7, 6.5 Hz, 2 H) 3.50-3.59 (m, 4 H) 3.80 (dd, J = 12.7, 3.5 Hz, 2 H) 3.83-3.90 (m, 2 H) 4.65-4.90 (m, 2 H) 7.07 (br s, 1 H) 7.11 (br s, 1 H) 7.26 (d, J = 9.5 Hz, 1 H) 7.40 (d, J = 9.5 Hz, 1 H) 10.05-10.65 (m, 1 H) |
| 201 | 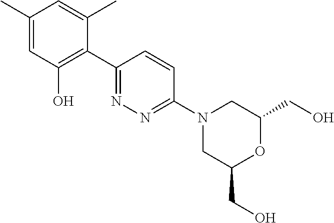 [SMILES: Cc1cc(C)c(-c2ccc(N3C[C@H](CO)O[C@@H](CO)C3)nn2)c(O)c1] | 346 0.89 U | 1H NMR (400 MHz, DMSO-d6, 30° C.) δ ppm 2.03 (s, 3 H) 2.22 (s, 3 H) 3.43 (br dd, J = 12.7, 6.5 Hz, 2 H) 3.50-3.58 (m, 4 H) 3.77 (dd, J = 12.7, 3.5 Hz, 2 H) 3.81-3.89 (m, 2 H) 4.65-4.87 (m, 2 H) 6.57 (br s, 1 H) 6.58 (br s, 1 H) 7.22 (d, J = 9.5 Hz, 1 H) 7.34 (d, J = 9.5 Hz, 1 H) 9.41 (br s, 1 H) |
| 203 | 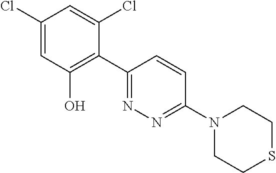 [SMILES: Oc1cc(Cl)cc(Cl)c1-c1ccc(N2CCSCC2)nn1] | 342 1.60 U | 1H NMR (500 MHz, DMSO-d6, 27° C.) δ ppm 2.66- 2.70 (m, 4 H) 4.00-4.04 (m, 4 H) 6.96 (d, J = 2.0 Hz, 1 H) 7.13 (d, J = 2.0 Hz, 1 H) 7.32 (d, J = 9.5 Hz, 1 H) 7.37 (d, J = 9.5 Hz, 1 H) 10.40-10.70 (m, 1 H) |
| 204 | 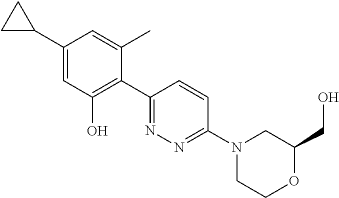 [SMILES: Cc1cc(C2CC2)cc(O)c1-c1ccc(N2CCO[C@H](CO)C2)nn1] | 342 1.05 U | 1H NMR (400 MHz, DMSO-d6, 30° C.) δ ppm 0.58- 0.68 (m, 2 H) 0.87-0.98 (m, 2 H) 1.78-1.87 (m, 1 H) 2.03 (s, 3 H) 2.70 (dd, J = 12.7, 10.0 Hz, 1 H) 2.96 (td, J = 12.7, 2.6 Hz, 1 H) 3.45-3.65 (m, 4 H) 3.99 (dd, J = 11.6, 2.6 Hz, 1 H) 4.12 (br d, J = 12.7 Hz, 1 H) 4.31 (br d, J = 12.7 Hz, 1 H) 4.81 (t, J = 5.4 Hz, 1 H) 6.46 (br s, 1 H) 6.48 (br s, 1 H) 7.26 (d, J = 9.5 Hz, 1 H) 7.35 (d, J = 9.5 Hz, 1 H) 9.38 (s, 1 H) |
| 206 | 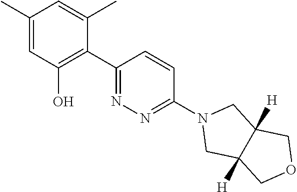 [SMILES: Cc1cc(C)c(-c2ccc(N3C[C@H]4COC[C@H]4C3)nn2)c(O)c1] | 312 0.92 U | 1H NMR (500 MHz, DMSO-d6, 27° C.) δ ppm 2.03 (s, 3 H) 2.22 (s, 3 H) 3.03-3.10 (m, 2 H) 3.46 (dd, J = 10.9, 3.3 Hz, 2 H) 3.60 (dd, J = 8.9, 3.7 Hz, 2 H) 3.66 (br dd, J = 10.9, 7.7 Hz, 2 H) 3.88 (br dd, J = 8.9, 6.9 Hz, 2 H) 6.56 (br s, 1 H) 6.57 (br s, 1 H) 6.94 (d, J = 9.4 Hz, 1 H) 7.30 (d, J = 9.4 Hz, 1 H) 9.37 (s, 1 H) |
| 207 | 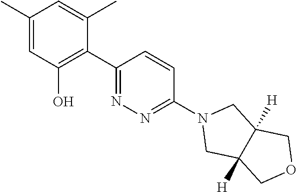 [SMILES: Cc1cc(C)c(-c2ccc(N3C[C@H]4COC[C@@H]4C3)nn2)c(O)c1] | 312 0.91 U | 1H NMR (500 MHz, DMSO-d6, 27° C.) δ ppm 2.04 (s, 3 H) 2.22 (s, 3 H) 2.52-2.61 (m, 2 H) 3.22 (br dd, J = 10.6, 9.4 Hz, 2 H) 3.47 (br dd, J = 10.6, 7.2 Hz, 2 H) 3.78-3.84 (m, 2 H) 3.91 (br t, J = 6.8 Hz, 2 H) 6.56 (br s, 1 H) 6.57 (br s, 1 H) 6.86 (d, J = 9.4 Hz, 1 H) 7.29 (d, J = 9.4 Hz, 1 H) 9.37 (s, 1 H) |
| 208 | 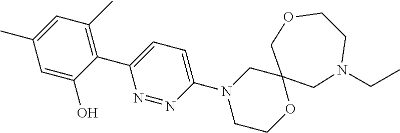 [SMILES: CCN1CCOCC2(C1)CN(c1ccc(-c3c(C)cc(C)cc3O)nn1)CCO2] | 399.4 2.90 E | ¹H NMR (400 MHz, DMSO-d6) δ (ppm): 9.33 (s, 1H), 7.27 (d, J = 9.4 Hz, 1H), 7.18 (d, J = 9.4 Hz, 1H), 6.51 (d, J = 4.3 Hz, 2H), 3.67 (q, J = 9.4 Hz, 6H), 3.55-3.44 (m, 2H), 3.13 (d, J = 13.0 Hz, 1H), 2.83 (d, J = 13.4 Hz, 1H), 2.56 (s, 2H), 2.50 (s, 1H), 2.15 (s, 3H), 1.95 (s, 3H), 0.86 (t, J = 7.0 Hz, 3H). 3 protons hidden under DMSO and water peaks (confirmed by HSQC). |
| 209 | 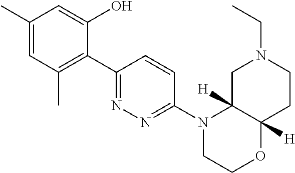 [SMILES: CCN1CC[C@@H]2OCCN(c3ccc(-c4c(C)cc(C)cc4O)nn3)[C@@H]2C1] | 369 0.87 U | 1H NMR (400 MHz, DMSO-d6, 30° C.) δ ppm 2.04 (s, 3 H) 2.74 (dd, J = 12.7, 10.0 Hz, 1 H) 3.00 (td, J = 12.7, 2.6 Hz, 1 H) 3.44-3.65 (m, 4 H) 3.99 (dd, J = 11.6, 2.0 Hz, 1 H) 4.15 (br d, J = 12.7 Hz, 1 H) 4.34 (br d, J = 12.7 Hz, 1 H) 4.80 (br s, 1 H) 6.97 (d, J = 6.5 Hz, 1 H) 7.31 (d, J = 9.5 Hz, 1 H) 7.41 (d, J = 9.5 Hz, 1 H) 9.21-10.32 (m, 1 H) |
| 210 | 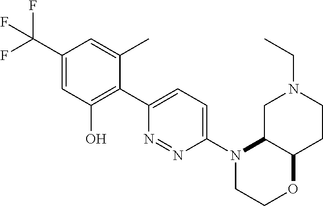 | 423 1.03 U | 1H NMR (400 MHz, DMSO-d6, 30° C.) δ ppm 0.98 (t, J = 7.0 Hz, 3 H) 1.79-1.91 (m, 2 H) 2.06-2.14 (m, 1 H) 2.13 (s, 3 H) 2.29-2.45 (m, 3 H) 2.55-2.66 (m, 2 H) 3.18 (td, J = 11.8, 3.8 Hz, 1 H) 3.63 (td, J = 11.8, 3.8 Hz, 1 H) 3.73-3.79 (m, 1 H) 3.96 (br d, J = 11.8 Hz, 1 H) 4.05 (dd, J = 11.8, 3.8 Hz, 1 H) 4.37-4.47 (m, 1 H) 7.07 (br s, 1 H) 7.12 (br s, 1 H) 7.24 (d, J = 9.5 Hz, 1 H) 7.40 (d, J = 9.5 Hz, 1 H) 10.13 (br s, 1 H) |
| 211 | 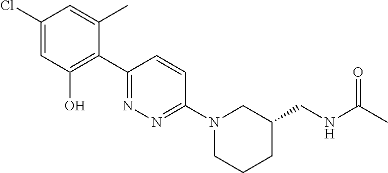 [SMILES: CC(=O)NC[C@@H]1CCCN(c2ccc(-c3c(C)cc(Cl)cc3O)nn2)C1] | 375 1.06 U | 1H NMR (400 MHz, DMSO-d6, 30° C.) δ ppm 1.15- 1.28 (m, 1 H) 1.42-1.54 (m, 1 H) 1.62-1.85 (m, 3 H) 1.84 (s, 3 H) 2.06 (s, 3 H) 2.69 (dd, J = 13.0, 10.5 Hz, 1 H) 2.92-3.09 (m, 3 H) 4.21 (br d, J = 13.0 Hz, 1 H) 4.32 (br dd, J = 13.0, 3.5 Hz, 1 H) 6.81 (d, J = 2.0 Hz, 1 H) 6.83 (d, J = 2.0 Hz, 1 H) 7.25 (d, J = 9.5 Hz, 1 H) 7.31 (d, J = 9.5 Hz, 1 H) 7.90 (t, J = 5.9 Hz, 1 H) 9.80-10.20 (m, 1 H) |
| 212 | 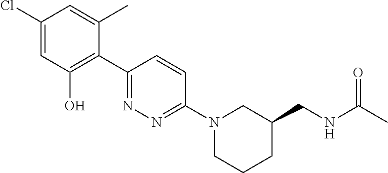 [SMILES: CC(=O)NC[C@H]1CCCN(c2ccc(-c3c(C)cc(Cl)cc3O)nn2)C1] | 375 1.06 U | 1H NMR (400 MHz, DMSO-d6, 30° C.) δ ppm 1.18- 1.34 (m, 1 H) 1.46-1.58 (m, 1 H) 1.68-1.85 (m, 3 H) 1.84 (s, 3 H) 2.10 (s, 3 H) 2.84 (dd, J = 13.0, 10.5 Hz, 1 H) 2.98-3.14 (m, 3 H) 4.19 (br d, J = 13.0 Hz, 1 H) 4.25 (br dd, J = 13.0, 3.5 Hz, 1 H) 6.85 (d, J = 2.0 Hz, 1 H) 6.88 (d, J = 2.0 Hz, 1 H) 7.52-7.68 (m, 2 H) 7.90 (t, J = 5.9 Hz, 1 H) 10.00-10;50 (m, 1 H) |
| 213 | 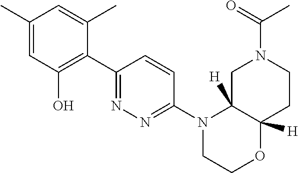 [SMILES: CC(=O)N1CC[C@@H]2OCCN(c3ccc(-c4c(C)cc(C)cc4O)nn3)[C@@H]2C1] | 383 1.09 U | 1H NMR (400 MHz, DMSO-d6, 100° C.) δ ppm 1.81- 1.89 (m, 2 H) 2.03 (s, 3 H) 2.09 (s, 3 H) 2.25 (s, 3 H) 2.74-4.49 (m, 4 H) 3.26 (td, J = 12.7, 3.8 Hz, 1 H) 3.72 (td, J = 11.8, 3.2 Hz, 1 H) 3.87-3.93 (m, 1 H) 4.00 (br d, J = 12.8 Hz, 1 H) 4.11 (dd, J = 11.8, 3.2 Hz, 1 H) 4.24-4.44 (m, 1 H) 6.60 (br s, 1 H) 6.61 (br s, 1 H) 7.22 (br d, J = 9.5 Hz, 1 H) 7.39 (d, J = 9.5 Hz, 1 H) 9.10-9.20 (m, 1 H) |
| 214 | 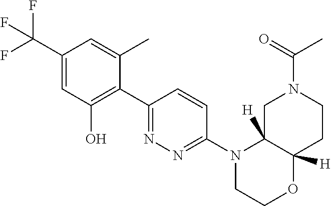 [SMILES: CC(=O)N1CC[C@@H]2OCCN(c3ccc(-c4c(C)cc(C(F)(F)F)cc4O)nn3)[C@@H]2C1] | 437 1.38 U | 1H NMR (400 MHz, DMSO-d6, 100° C.) δ ppm 1.81- 1.91 (m, 2 H) 2.03 (s, 3 H) 2.17 (s, 3 H) 2.72-4.48 (m, 4 H) 3.28 (td, J = 12.7, 3.8 Hz, 1 H) 3.73 (td, J = 11.8, 3.2 Hz, 1 H) 3.88-3.94 (m, 1 H) 4.02 (br d, J = 11.8 Hz, 1 H) 4.11 (br dd, J = 11.8, 3.2 Hz, 1 H) 4.28-4.44 (m, 1 H) 7.07-7.10 (m, 2 H) 7.26 (br d, J = 9.5 Hz, 1 H) 7.43 (d, J = 9.5 Hz, 1 H) 9.15-10.45 (m, 1 H) |
| 215 | 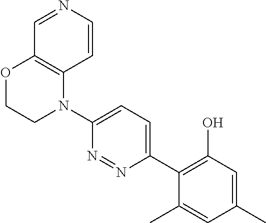 [SMILES: Cc1cc(C)c(-c2ccc(N3CCOc4cnccc43)nn2)c(O)c1] | 335 0.88 U | 1H NMR (400 MHz, DMSO-d6, 30° C.) δ ppm 2.07 (s, 3 H) 2.25 (s, 3 H) 4.08-4.13 (m, 2 H) 4.38-4.43 (m, 2 H) 6.62 (br s, 1 H) 6.63 (br s, 1 H) 7.45 (br d, J = 5.5 Hz, 1 H) 7.60 (d, J = 9.5 Hz, 1 H) 7.71 (d, J = 9.5 Hz, 1 H) 7.94 (br d, J = 5.5 Hz, 1 H) 8.13 (br s, 1 H) 9.43 (br s, 1 H) |
| 216 | 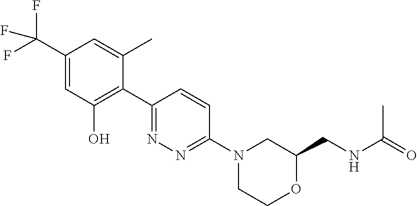 [SMILES: CC(=O)NC[C@@H]1CN(c2ccc(-c3c(C)cc(C(F)(F)F)cc3O)nn2)CCO1] | 411 1.24 U | 1H NMR (400 MHz, DMSO-d6, 30° C.) δ ppm 1.85 (s, 3 H) 2.13 (s, 3 H) 2.70 (dd, J = 12.7, 10.5 Hz, 1 H) 3.01 (td, J = 12.7, 3.5 Hz, 1 H) 3.24 (t, J = 5.7 Hz, 2 H) 3.52-3.65 (m, 2 H) 4.01 (br d, J = 11.5 Hz, 1 H) 4.13 (br d, J = 12.7 Hz, 1 H) 4.31 (br d, J = 12.7 Hz, 1 H) 7.06 (br s, 1 H) 7.12 (br s, 1 H) 7.32 (d, J = 9.5 Hz, 1 H) 7.42 (d, J = 9.5 Hz, 1 H) 8.01 (t, J = 5.7 Hz, 1 H) 10.12 (s, 1 H) |
| 217 | 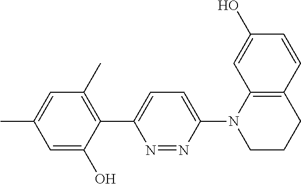 [SMILES: Cc1cc(C)c(-c2ccc(N3CCCc4ccc(O)cc43)nn2)c(O)c1] | 348 1.32 U | 1H NMR (400 MHz, DMSO-d6, 30° C.) δ ppm 1.92 (quin, J = 6.3 Hz, 2 H) 2.07 (s, 3 H) 2.24 (s, 3 H) 2.66 (br t, J = 6.3 Hz, 2 H) 3.94 (br t, J = 6.3 Hz, 2 H) 6.40 (dd, J = 8.2, 2.0 Hz, 1 H) 6.60 (br s, 1 H) 6.61 (br s, 1 H) 6.65 (d, J = 2.0 Hz, 1 H) 6.98 (d, J = 8.2 Hz, 1 H) 7.37 (d, J = 9.3 Hz, 1 H) 7.51 (d, J = 9.3 Hz, 1 H) 9.17 (br s, 1 H) 9.41 (br s, 1 H) |
| 219 | 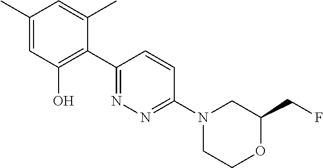 [SMILES: Cc1cc(C)c(-c2ccc(N3CCO[C@H](CF)C3)nn2)c(O)c1] | 318 1.10 U | 1H NMR (400 MHz, DMSO-d6, 30° C.) δ ppm 2.03 (s, 3 H) 2.22 (s, 3 H) 2.82 (dd, J = 12.7, 10.8 Hz, 1 H) 2.95-3.04 (m, 1 H) 3.66 (td, J = 11.6, 2.6 Hz, 1 H) 3.75-3.88 (m, 1 H) 4.03 (dd, J = 11.6, 2.6 Hz, 1 H) 4.13 (br d, J = 12.7 Hz, 1 H) 4.31 (br d, J = 12.7 Hz, 1 H) 4.44-4.66 (m, 2 H) 6.57 (br s, 1 H) 6.58 (br s, 1 H) 7.29 (d, J = 9.5 Hz, 1 H) 7.36 (d, J = 9.5 Hz, 1 H) 9.36 (br s, 1 H) |
| 220 | 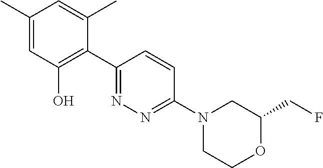 [SMILES: Cc1cc(C)c(-c2ccc(N3CCO[C@@H](CF)C3)nn2)c(O)c1] | 318 1.10 U | 1H NMR (400 MHz, DMSO-d6, 30° C.) δ ppm 2.03 (s, 3 H) 2.22 (s, 3 H) 2.82 (dd, J = 12.7, 10.8 Hz, 1 H) 2.95-3.04 (m, 1 H) 3.66 (td, J = 11.6, 2.6 Hz, 1 H) 3.75-3.87 (m, 1 H) 4.00-4.06 (m, 1 H) 4.13 (br d, J = 12.7 Hz, 1 H) 4.31 (br d, J = 12.7 Hz, 1 H) 4.44- 4.67 (m, 2 H) 6.57 (br s, 1 H) 6.58 (br s, 1 H) 7.29 (d, J = 9.5 Hz, 1 H) 7.36 (d, J = 9.5 Hz, 1 H) 9.36 (br s, 1 H) |
| 221 | 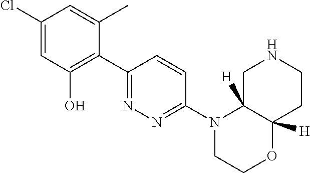 [SMILES: Cc1cc(Cl)cc(O)c1-c1ccc(N2CCO[C@H]3CCNC[C@H]32)nn1] | 361 0.91 U | 1H NMR (400 MHz, DMSO-d6, 30° C.) δ ppm 1.70- 1.82 (m, 2 H) 2.05 (s, 3 H) 2.59-2.70 (m, 3 H) 2.92 (br t, J = 11.8 Hz, 1 H) 3.15 (td, J = 11.8, 3.8 Hz, 1 H) 3.61 (td, J = 11.8, 3.8 Hz, 1 H) 3.77-3.89 (m, 1 H) 3.98 (br d, J = 11.8 Hz, 1 H) 4.06 (dd, J = 11.8, 3.8 Hz, 1 H) 4.14-4.28 (m, 1 H) 6.81 (d, J = 2.0 Hz, 1 H) 6.84 (d, J = 2.0 Hz, 1 H) 7.21 (d, J = 9.5 Hz, 1 H) 7.35 (d, J = 9.5 Hz, 1 H) 8.96-10.86 (m, 1 H) |
| 222 | 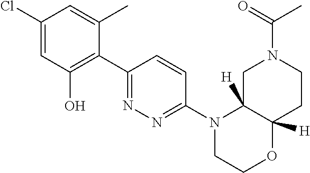 [SMILES: CC(=O)N1CC[C@@H]2OCCN(c3ccc(-c4c(C)cc(Cl)cc4O)nn3)[C@@H]2C1] | 403 1.24 U | 1H NMR (400 MHz, DMSO-d6, 100° C.) δ ppm 1.79- 1.88 (m, 2 H) 2.01 (s, 3 H) 2.08 (s, 3 H) 2.73-4.53 (m, 4 H) 3.25 (td, J = 12.7, 3.8 Hz, 1 H) 3.70 (td, J = 11.8, 3.2 Hz, 1 H) 3.83-3.93 (m, 1 H) 3.99 (br d, J = 11.8 Hz, 1 H) 4.09 (dd, J = 11.8, 3.2 Hz, 1 H) 4.24-4.39 (m, 1 H) 6.79-6.82 (m, 2 H) 7.21 (br d, J = 9.5 Hz, 1 H) 7.37 (d, J = 9.5 Hz, 1 H) 9.35-9.95 (m, 1 H) |
| 223 | 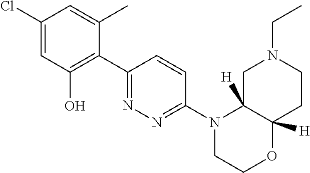 [SMILES: CCN1CC[C@@H]2OCCN(c3ccc(-c4c(C)cc(Cl)cc4O)nn3)[C@@H]2C1] | 389 0.94 U | 1H NMR (400 MHz, DMSO-d6, 30° C.) δ ppm 0.97 (t, J = 7.1 Hz, 3 H) 1.78-1.90 (m, 2 H) 2.05 (s, 3 H) 2.05-2.16 (m, 1 H) 2.32-2.43 (m, 3 H) 2.56-2.65 (m, 2 H) 3.16 (td, J = 11.8, 3.8 Hz, 1 H) 3.62 (td, J = 11.8, 3.8 Hz, 1 H) 3.71-3.79 (m, 1 H) 3.94 (br d, J = 11.8 Hz, 1 H) 4.05 (dd, J = 11.8, 3.8 Hz, 1 H) 4.34-4.46 (m, 1 H) 6.81 (d, J = 2.0 Hz, 1 H) 6.84 (d, J = 2.0 Hz, 1 H) 7.22 (d, J = 9.5 Hz, 1 H) 7.36 (d, J = 9.5 Hz, 1 H) 9.96 (s, 1 H) |
| 224 | 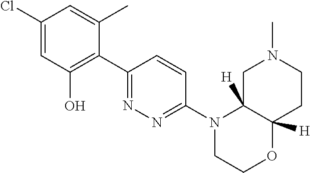 [SMILES: Cc1cc(Cl)cc(O)c1-c1ccc(N2CCO[C@H]3CCN(C)C[C@H]32)nn1] | 375 0.92 U | 1H NMR (400 MHz, DMSO-d6, 30° C.) δ ppm 1.76- 1.92 (m, 2 H) 2.05 (s, 3 H) 2.05-2.14 (m, 1 H) 2.18 (s, 3 H) 2.29-2.36 (m, 1 H) 2.51-2.54 (m partially hidden, 2 H) 3.16 (td, J = 11.8, 3.8 Hz, 1 H) 3.62 (td, J = 11.8, 3.8 Hz, 1 H) 3.71-3.76 (m, 1 H) 3.93 (br d, J = 11.8 Hz, 1 H) 4.05 (dd, J = 11.8, 3.8 Hz, 1 H) 4.37-4.45 (m, 1 H) 6.81 (d, J = 1.8 Hz, 1 H) 6.84 (d, J = 1.8 Hz, 1 H) 7.22 (d, J = 9.5 Hz, 1 H) 7.35 (d, J = 9.5 Hz, 1 H) 9.90-10.00 (m, 1 H) |
| 225 | 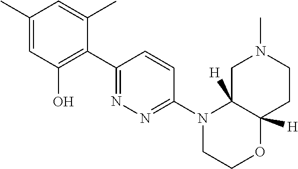 [SMILES: Cc1cc(C)c(-c2ccc(N3CCO[C@H]4CCN(C)C[C@H]43)nn2)c(O)c1] | 355 0.86 U | 1H NMR (400 MHz, DMSO-d6, 30° C.) δ ppm 1.76- 1.93 (m, 2 H) 2.03 (s, 3 H) 2.05-2.14 (m, 1 H) 2.18 (s, 3 H) 2.22 (s, 3 H) 2.29-2.37 (m, 1 H) 2.51-2.57 (m partially hidden, 2 H) 3.16 (td, J = 11.8, 3.8 Hz, 1 H) 3.63 (td, J = 11.8, 3.8 Hz, 1 H) 3.71-3.77 (m, 1 H) 3.92 (br d, J = 11.8 Hz, 1 H) 4.05 (dd, J = 11.8, 3.8 Hz, 1 H) 4.36-4.44 (m, 1 H) 6.57 (br s, 1 H) 6.58 (br s, 1 H) 7.20 (d, J = 9.5 Hz, 1 H) 7.34 (d, J = 9.5 Hz, 1 H) 9.36 (s, 1 H) |
| 226 | 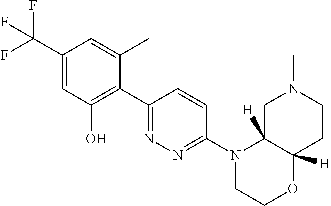 [SMILES: Cc1cc(C(F)(F)F)cc(O)c1-c1ccc(N2CCO[C@H]3CCN(C)C[C@H]32)nn1] | 409 1.03 U | 1H NMR (400 MHz, DMSO-d6, 30° C.) δ ppm 1.77- 1.93 (m, 2 H) 2.04-2.12 (m, 1 H) 2.13 (s, 3 H) 2.18 (s, 3 H) 2.30-2.38 (m, 1 H) 2.52-2.56 (m partially hidden, 2 H) 3.17 (td, J = 11.8, 3.8 Hz, 1 H) 3.63 (td, J = 11.8, 3.8 Hz, 1 H) 3.71-3.78 (m, 1 H) 3.95 (br d, J = 11.8 Hz, 1 H) 4.05 (dd, J = 11.8, 3.8 Hz, 1 H) 4.38-4.47 (m, 1 H) 7.07 (br s, 1 H) 7.12 (br s, 1 H) 7.25 (d, J = 9.5 Hz, 1 H) 7.40 (d, J = 9.5 Hz, 1 H) 10.12 (br s, 1 H) |
| 227 | 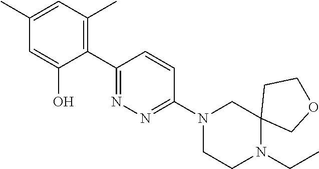 [SMILES: CCN1CCN(c2ccc(-c3c(C)cc(C)cc3O)nn2)CC12CCOC2] | 369.3 2.75 E | ¹H NMR (500 MHz, DMSO-d₆) δ (ppm): 9.39 (s, 1H), 7.33 (s, 2H), 6.58 (d, J = 6.3 Hz, 2H), 3.94- 3.86 (m, 1H), 3.75 (dt, J = 15.4, 9.2 Hz, 4H), 3.43- 3.36 (m, 2H), 3.29-3.26 (m, 1H), 2.74 (d, J = 12.0 Hz, 1H), 2.60 (dd, J = 13.1, 6.9 Hz, 2H), 2.33 (dq, J = 13.5, 7.5 Hz, 1H), 2.23 (s, 3H), 2.04 (s, 3H), 1.96 (dt, J = 16.8, 8.5 Hz, 1H), 1.70 (dq, J = 12.8, 5.5 Hz, 1H), 1.05 (t, J = 7.1 Hz, 3H). |
| 228 | 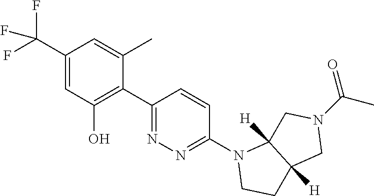 [SMILES: CC(=O)N1C[C@@H]2CCN(c3ccc(-c4c(C)cc(C(F)(F)F)cc4O)nn3)[C@@H]2C1] | 407.3 2.73 E | ¹H NMR (500 MHz, DMSO-d₆) δ (ppm) : 10.13 (s, 1H), 7.38 (t, J = 8.7 Hz, 1H), 7.12 (s, 1H), 7.06 (s, 1H), 6.99 (dd, J = 9.4, 3.7 Hz, 1H), 4.66-4.45 (m, 1H), 4.15-3.33 (m, 6H), 3.21-2.97 (m, 1H), 2.26- 2.05 (m, 4H), 2.02-1.78 (m, 4H). |
| 229 | 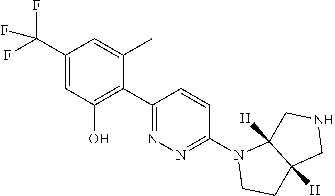 [SMILES: Cc1cc(C(F)(F)F)cc(O)c1-c1ccc(N2CC[C@H]3CNC[C@H]32)nn1] | 365.2 2.50 E | ¹H NMR (500 MHz, DMSO-d₆) δ (ppm) : 7.34 (d, J = 9.3 Hz, 1H), 7.12 (s, 1H), 7.06 (s, 1H), 6.94 (d, J = 9.4 Hz, 1H), 4.34 (s, 1H), 3.61 (tt, J = 13.9, 7.7 Hz, 2H), 2.96 (dd, J = 11.8, 5.8 Hz, 1H), 2.90- 2.79 (m, 3H), 2.70 (d, J = 7.6 Hz, 1H), 2.14 (s, 3H), 2.12-2.06 (m, 1H), 1.84 (dd, J = 12.3, 6.0 Hz, 1H). (OH and NH not visible) |
| 230 | 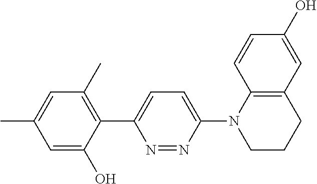 [SMILES: Cc1cc(C)c(-c2ccc(N3CCCc4cc(O)ccc43)nn2)c(O)c1] | 348 1.20 U | 1H NMR (400 MHz, DMSO-d6, 30° C.) δ ppm 1.90 (quin, J = 6.4 Hz, 2 H) 2.06 (s, 3 H) 2.22 (s, 3 H) 2.66 (t, J = 6.4 Hz, 2 H) 3.95 (t, J = 6.4 Hz, 2 H) 6.55-6.61 (m, 3 H) 6.63 (d, J = 2.8 Hz, 1 H) 7.07 (d, J = 8.5 Hz, 1 H) 7.27 (d, J = 9.5 Hz, 1 H) 7.31 (d, J = 9.5 Hz, 1 H) 9.10-9.50 (m, 2 H) |
| 231 | 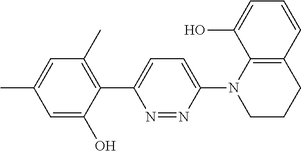 [SMILES: Cc1cc(C)c(-c2ccc(N3CCCc4cccc(O)c43)nn2)c(O)c1] | 348 1.23 U | 1H NMR (400 MHz, DMSO-d6, 30° C.) δ ppm 1.92 (quin, J = 6.4 Hz, 2 H) 2.06 (s, 3 H) 2.23 (s, 3 H) 2.69 (t, J = 6.4 Hz, 2 H) 4.06 (t, J = 6.4 Hz, 2 H) 6.58 (br s, 1 H) 6.59 (br s, 1 H) 6.64 (d, J = 9.5 Hz, 1 H) 6.70- 6.78 (m, 2 H) 6.95 (t, J = 7.7 Hz, 1 H) 7.28 (d, J = 9.5 Hz, 1 H) 9.45 (br s, 1 H) 9.64 (br s, 1 H) |
| 232 | 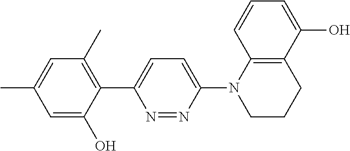 [SMILES: Cc1cc(C)c(-c2ccc(N3CCCc4c(O)cccc43)nn2)c(O)c1] | 348 1.30 U | 1H NMR (400 MHz, DMSO-d6, 30° C.) δ ppm 1.87- 1.96 (m, 2 H) 2.07 (s, 3 H) 2.23 (s, 3 H) 2.65 (t, J = 6.4 Hz, 2 H) 3.96-4.04 (m, 2 H) 6.52 (d, J = 7.7 Hz, 1 H) 6.59 (br s, 1 H) 6.60 (br s, 1 H) 6.65 (d, J = 7.7 Hz, 1 H) 6.92 (t, J = 7.7 Hz, 1 H) 7.33 (d, J = 9.5 Hz, 1 H) 7.45 (d, J = 9.5 Hz, 1 H) 9.41 (br s, 2 H) |
| 233 | 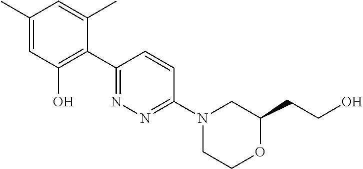 [SMILES: Cc1cc(C)c(-c2ccc(N3CCO[C@H](CCO)C3)nn2)c(O)c1] | 330 0.96 U | 1H NMR (400 MHz, DMSO-d6, 30° C.) δ ppm 1.60- 1.71 (m, 2 H) 2.03 (s, 3 H) 2.22 (s, 3 H) 2.68 (dd, J = 12.7, 10.0 Hz, 1 H) 2.96 (td, J = 12.7, 2.6 Hz, 1 H) 3.50-3.66 (m, 4 H) 3.97 (dd, J = 11.6, 2.6 Hz, 1 H) 4.13 (br d, J = 12.7 Hz, 1 H) 4.24 (br d, J = 12.7 Hz, 1 H) 4.47 (t, J = 5.4 Hz, 1 H) 6.57 (br s, 1 H) 6.58 (br s, 1 H) 7.28 (d, J = 9.5 Hz, 1 H) 7.34 (d, J = 9.5 Hz, 1 H) 9.36 (s, 1 H) |
| 234 | 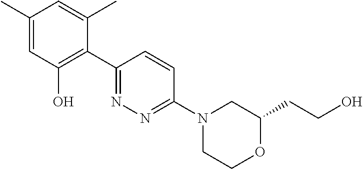 [SMILES: Cc1cc(C)c(-c2ccc(N3CCO[C@@H](CCO)C3)nn2)c(O)c1] | 330 0.96 U | 1H NMR (400 MHz, DMSO-d6, 30° C.) δ ppm 1.62- 1.71 (m, 2 H) 2.03 (s, 3 H) 2.22 (s, 3 H) 2.68 (dd, J = 12.7, 10.0 Hz, 1 H) 2.96 (td, J = 12.7, 2.6 Hz, 1 H) 3.50-3.66 (m, 4 H) 3.97 (dd, J = 11.6, 2.6 Hz, 1 H) 4.13 (br d, J = 12.7 Hz, 1 H) 4.24 (br d, J = 12.7 Hz, 1 H) 4.47 (t, J = 5.4 Hz, 1 H) 6.57 (br s, 1 H) 6.58 (br s, 1 H) 7.28 (d, J = 9.5 Hz, 1 H) 7.34 (d, J = 9.5 Hz, 1 H) 9.36 (br s, 1 H) |
| 235 | 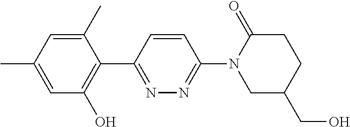 [SMILES: Cc1cc(C)c(-c2ccc(N3CC(CO)CCC3=O)nn2)c(O)c1] | 328 1,16 U | 1H NMR (400 MHz, DMSO-d6, 30° C.) δ ppm 1.55- 1.66 (m, 1 H) 1.88-1.96 (m, 1 H) 2.02 (s, 3 H) 2.08- 2.19 (m, 1 H) 2.24 (s, 3 H) 2.54-2.61 (m, 2 H) 3.39- 3.53 (m, 2 H) 3.74 (dd, J = 12.7, 10.0 Hz, 1 H) 4.14- 4.25 (m, 1 H) 4.75 (t, J = 5.4 Hz, 1 H) 6.61 (br s, 1 H) 6.62 (br s, 1 H) 7.58 (d, J = 9.5 Hz, 1 H) 7.97 (d, J = 9.5 Hz, 1 H) 9.43 (s, 1 H) |
| 236 | 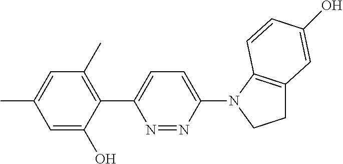 [SMILES: Cc1cc(C)c(-c2ccc(N3CCc4cc(O)ccc43)nn2)c(O)c1] | 334 1.17 U | 1H NMR (400 MHz, DMSO-d6, 30° C.) δ ppm 2.06 (s, 3 H) 2.24 (s, 3 H) 3.18 (t, J = 8.5 Hz, 2 H) 4.05 (t, J = 8.5 Hz, 2 H) 6.57-6.61 (m, 3 H) 6.69-6.72 (m, 1 H) 7.18 (d, J = 9.5 Hz, 1 H) 7.43 (d, J = 9.5 Hz, 1 H) 8.17 (d, J = 8.5 Hz, 1 H) 8.90-9.10 (m, 1 H) 9.30- 9.50 (m, 1 H) |
| 237 | 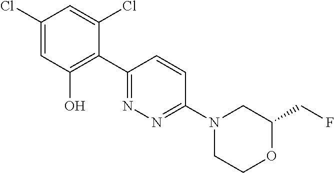 [SMILES: Oc1cc(Cl)cc(Cl)c1-c1ccc(N2CCO[C@@H](CF)C2)nn1] | 358 1.50 U | 1H NMR (400 MHz, DMSO-d6, 30° C.) δ ppm 2.85 (dd, J = 12.7, 11.0 Hz, 1 H) 2.97-3.06 (m, 1 H) 3.66 (td, J = 11.6, 2.6 Hz, 1 H) 3.75-3.87 (m, 1 H) 4.00- 4.07 (m, 1 H) 4.17 (br d, J = 12.7 Hz, 1 H) 4.34 (br d, J = 12.7 Hz, 1 H) 4.45-4.65 (m, 2 H) 6.96 (d, J = 2.0 Hz, 1 H) 7.14 (d, J = 2.0 Hz, 1 H) 7.33 (d, J = 9.5 Hz, 1 H) 7.42 (d, J = 9.5 Hz, 1 H) 10.40-10.70 (m, 1 H) |
| 238 | 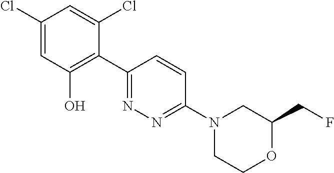 [SMILES: Oc1cc(Cl)cc(Cl)c1-c1ccc(N2CCO[C@H](CF)C2)nn1] | 358 1.50 U | 1H NMR (400 MHz, DMSO-d6, 30° C.) δ ppm 2.85 (dd, J = 12.7, 11.0 Hz, 1 H) 2.98-3.06 (m, 1 H) 3.66 (td, J = 11.6, 2.6 Hz, 1 H) 3.75-3.87 (m, 1 H) 4.04 (dd, J = 11.6, 2.6 Hz, 1 H) 4.17 (br d, J = 12.7 Hz, 1 H) 4.34 (br d, J = 12.7 Hz, 1 H) 4.45-4.65 (m, 2 H) 6.96 (d, J = 2.0 Hz, 1 H) 7.14 (d, J = 2.0 Hz, 1 H) 7.33 (d, J = 9.5 Hz, 1 H) 7.42 (d, J = 9.5 Hz, 1 H) 10.40-10.70 (m, 1 H) |
| 239 | 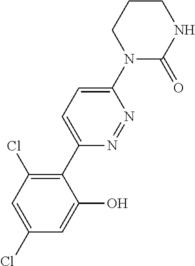 [SMILES: O=C1NCCCN1c1ccc(-c2c(O)cc(Cl)cc2Cl)nn1] | 339 1.35 U | 1H NMR (400 MHz, DMSO-d6, 30° C.) δ ppm 1.98- 2.04 (m, 2 H) 3.22-3.33 (m partially hidden, 2 H) 4.03-4.08 (m, 2 H) 6.97 (br s, 1 H) 7.16 (br s, 2 H) 7.52 (d, J = 9.5 Hz, 1 H) 8.17 (d, J = 9.5 Hz, 1 H) 10.50-10.80 (m, 1 H) |
| 240 | 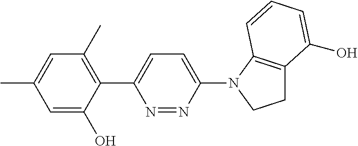 [SMILES: Cc1cc(C)c(-c2ccc(N3CCc4c(O)cccc43)nn2)c(O)c1] | 334 1.27 U | 1H NMR (400 MHz, DMSO-d6, 30° C.) δ ppm 2.06 (s, 3 H) 2.24 (s, 3 H) 3.09 (t, J = 8.5 Hz, 2 H) 4.11 (t, J = 8.5 Hz, 2 H) 6.42 (d, J = 7.7 Hz, 1 H) 6.60 (br s, 1 H) 6.60 (br s, 1 H) 7.00 (t, J = 7.7 Hz, 1 H) 7.27 (d, J = 9.5 Hz, 1 H) 7.47 (d, J = 9.5 Hz, 1 H) 7.78 (d, J = 7.7 Hz, 1 H) 9.37 (br s, 2 H) |
| 241 | 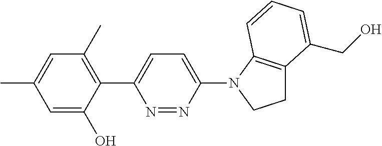 [SMILES: Cc1cc(C)c(-c2ccc(N3CCc4c(CO)cccc43)nn2)c(O)c1] | 348 1.37 U | 1H NMR (400 MHz, DMSO-d6, 30° C.) δ ppm 2.07 (s, 3 H) 2.24 (s, 3 H) 3.21 (t, J = 8.5 Hz, 2 H) 4.13 (t, J = 8.5 Hz, 2 H) 4.50 (d, J = 5.4 Hz, 2 H) 5.08 (br t, J = 5.4 Hz, 1 H) 6.60 (br s, 1 H) 6.61 (br s, 1 H) 6.95 (d, J = 7.7 Hz, 1 H) 7.18 (t, J = 7.7 Hz, 1 H) 7.29 (d, J = 9.5 Hz, 1 H) 7.49 (d, J = 9.5 Hz, 1 H) 8.25 (d, J = 7.7 Hz, 1 H) 9.38 (br s, 1 H) |
| 242 | 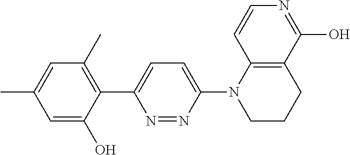 [SMILES: Cc1cc(C)c(-c2ccc(N3CCCc4c3ccnc4O)nn2)c(O)c1] | 349 1.28 U | 1H NMR (400 MHz, DMSO-d6, 30° C.) δ ppm 1.73- 1.82 (m, 2 H) 2.05 (s, 3 H) 2.25 (s, 3 H) 2.41 (t, J = 6.4 Hz, 2 H) 3.18-3.25 (m, 2 H) 5.90 (d, J = 7.8 Hz, 1 H) 6.63 (br s, 1 H) 6.64 (br s, 1 H) 6.97 (br s, 1 H) 7.69 (d, J = 9.5 Hz, 1 H) 7.72 (d, J = 7.8 Hz, 1 H) 8.05 (d, J = 9.5 Hz, 1 H) 9.53 (br s, 1 H) |
| 243 | 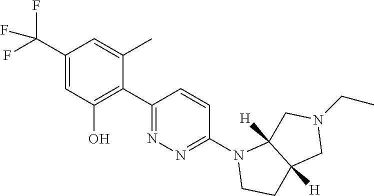 [SMILES: CCN1C[C@@H]2CCN(c3ccc(-c4c(C)cc(C(F)(F)F)cc4O)nn3)[C@@H]2C1] | 393.3 2.76 E | 1H NMR (400 MHz, DMSO-d6) δ (ppm) : 10.15 (s, 1H), 7.35 (d, J = 9.3 Hz, 1H), 7.16-7.10 (m, 1H), 7.11-7.04 (m, 1H), 6.97 (d, J = 9.3 Hz, 1H), 4.58- 4.32 (m, 1H), 3.83-3.42 (m, 2H), 3.05-2.79 (m, 1H), 2.76-2.57 (m, 3H), 2.48-2.32 (m, 3H), 2.13- 2.05 (m, 4H), 1.96-1.85 (m, 1H), 1.01 (t, J = 7.2 Hz, 3H).(compound cis confirmed by NMR NOESY) |
| 245 | 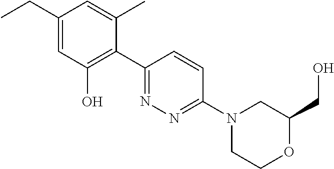 [SMILES: CCc1cc(C)c(-c2ccc(N3CCO[C@H](CO)C3)nn2)c(O)c1] | 330.2 2.63 E | ¹H NMR (500 MHz, DMSO-d₆) δ (ppm): 9.32 (s, 1H), 7.29 (d, J = 9.3 Hz, 1H), 7.20 (d, J = 9.4 Hz, 1H), 6.54 (d, J = 3.5 Hz, 2H), 4.75 (s, 1H), 4.25 (d, J = 12.8 Hz, 1H), 4.05 (d, J = 12.8 Hz, 1H), 3.92 (d, J = 11.3 Hz, 1H), 3.59-3.50 (m, 1H), 3.50-3.44 (m, 2H), 3.40 (d, J = 7.3 Hz, 1H), 2.91 (dt, J = 13.8, 6.9 Hz, 1H), 2.69-2.60 (m, 1H), 2.47 (d, J = 7.7 Hz, 2H), 1.98 (s, 3H), 1.11 (t, J = 7.6 Hz, 3H). |
| 246 | 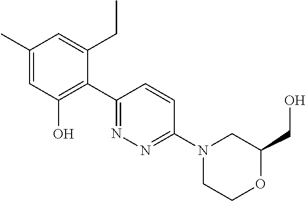 [SMILES: CCc1cc(C)cc(O)c1-c1ccc(N2CCO[C@H](CO)C2)nn1] | 330.2 2.54 E | ¹H NMR (500 MHz, DMSO-d₆) δ (ppm): 9.24 (s, 1H), 7.31 (d, J = 9.3 Hz, 1H), 7.25 (d, J = 9.4 Hz, 1H), 6.58 (d, J = 3.7 Hz, 2H), 4.82 (s, 1H), 4.31 (d, J = 12.8 Hz, 1H), 4.12 (d, J = 12.8 Hz, 1H), 3.99 (dd, J = 10.7, 3.3 Hz, 1H), 3.61 (td, J = 11.5, 2.7 Hz, 1H), 3.57-3.51 (m, 2H), 3.49-3.43 (m, 1H), 2.96 (td, J = 12.3, 3.5 Hz, 1H), 2.70 (dd, J = 12.8, 9.8 Hz, 1H), 2.35 (q, J = 7.4 Hz, 2H), 2.24 (s, 3H), 0.93 (t, J = 7.5 Hz, 3H). |
| 247 | 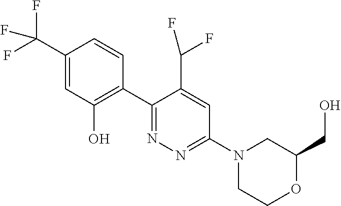 [SMILES: OC[C@@H]1CN(c2cc(C(F)F)c(-c3ccc(C(F)(F)F)cc3O)nn2)CCO1] | 406 1.40 U | 1H NMR (400 MHz, DMSO-d6, 30° C.) δ ppm 2.81 (dd, J = 12.7, 10.0 Hz, 1 H) 3.02-3.11 (m, 1 H) 3.45- 3.65 (m, 4 H) 4.00 (dd, J = 11.6, 2.6 Hz, 1 H) 4.26 (br d, J = 12.7 Hz, 1 H) 4.40 (br d, J = 12.7 Hz, 1 H) 4.83 (br t, J = 5.4 Hz, 1 H) 6.84 (t, J = 54.2 Hz, 1 H) 7.23 (s, 1 H) 7.27 (br d, J = 7.8 Hz, 1 H) 7.42 (s, 1 H) 7.50 (d, J = 7.8 Hz, 1 H) 10.55-10.80 (m, 1 H) |
| 248 | 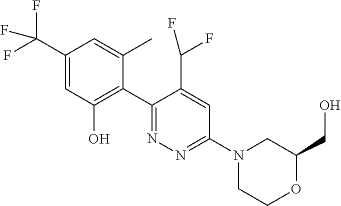 [SMILES: Cc1cc(C(F)(F)F)cc(O)c1-c1nnc(N2CCO[C@H](CO)C2)cc1C(F)F] | 420 1.43 U | 1H NMR (400 MHz, DMSO-d6, 30° C.) δ ppm 2.01 (s, 3 H) 2.82 (dd, J = 12.7, 10.0 Hz, 1 H) 3.02-3.12 (m, 1 H) 3.46-3.67 (m, 4 H) 3.96-4.06 (m, 1 H) 4.26 (br d, J = 12.7 Hz, 1 H) 4.41 (br d, J = 12.7 Hz, 1 H) 4.81 (br t, J = 5.4 Hz, 1 H) 6.62 (t, J = 54.0 Hz, 1 H) 7.07 (br s, 1 H) 7.16 (br s, 1 H) 7.46 (br s, 1 H) 10.20- 10.40 (m, 1 H) |
| 249 | 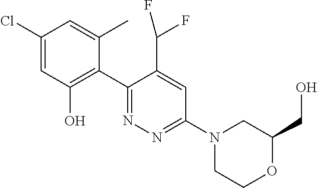 [SMILES: Cc1cc(Cl)cc(O)c1-c1nnc(N2CCO[C@H](CO)C2)cc1C(F)F] | 386 1.35 U | 1H NMR (400 MHz, DMSO-d6, 30° C.) δ ppm 1.92 (s, 3 H) 2.81 (dd, J = 12.7, 10.0 Hz, 1 H) 3.06 (td, J = 12.7, 2.6 Hz, 1 H) 3.45-3.66 (m, 4 H) 4.00 (dd, J = 11.6, 2.6 Hz, 1 H) 4.25 (br d, J = 12.7 Hz, 1 H) 4.39 (br d, J = 12.7 Hz, 1 H) 4.81 (br t, J = 5.4 Hz, 1 H) 6.57 (t, J = 54.3 Hz, 1 H) 6.82 (d, J = 2.0 Hz, 1 H) 6.88 (d, J = 2.0 Hz, 1 H) 7.43 (s, 1 H) 10.00- 10.15 (m, 1 H) |
| 250 | 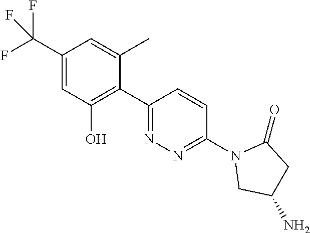 [SMILES: Cc1cc(C(F)(F)F)cc(O)c1-c1ccc(N2C[C@@H](N)CC2=O)nn1] | 353 0.93 U | 1H NMR (400 MHz, DMSO-d6, 30° C.) δ ppm 2.11 (s, 3 H) 2.31 (dd, J = 17.0, 3.5 Hz, 1 H) 2.87 (dd, J = 17.0, 6.7 Hz, 1 H) 3.69-3.78 (m, 1 H) 3.90 (dd, J = 11.0, 3.0 Hz, 1 H) 4.19 (dd, J = 11.0, 6.0 Hz, 1 H) 7.09 (br s, 1 H) 7.15 (br s, 1 H) 7.70 (d, J = 9.5 Hz, 1 H) 8.62 (d, J = 9.5 Hz, 1 H) |
| 251 | 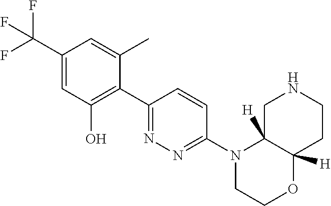 [SMILES: Cc1cc(C(F)(F)F)cc(O)c1-c1ccc(N2CCO[C@H]3CCNC[C@H]32)nn1] | 395 0.99 U | 1H NMR (400 MHz, DMSO-d6, 30° C.) δ ppm 1.70- 1.83 (m, 2 H) 2.13 (s, 3 H) 2.59-2.71 (m, 3 H) 2.93 (br t, J = 11.8 Hz, 1 H) 3.16 (td, J = 11.8, 3.8 Hz, 2 H) 3.62 (td, J = 11.8, 3.8 Hz, 1 H) 3.79-3.90 (m, 1 H) 3.99 (br d, J = 11.8 Hz, 1 H) 4.06 (dd, J = 11.8, 3.8 Hz, 1 H) 4.18-4.31 (m, 1 H) 7.06 (br s, 1 H) 7.11 (br s, 1 H) 7.25 (d, J = 9.5 Hz, 1 H) 7.40 (d, J = 9.5 Hz, 1 H) |
| 252 | 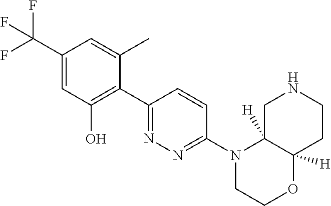 [SMILES: Cc1cc(C(F)(F)F)cc(O)c1-c1ccc(N2CCO[C@@H]3CCNC[C@@H]32)nn1] | 395 0.99 U | 1H NMR (400 MHz, DMSO-d6, 30° C.) δ ppm 1.80- 1.87 (m, 2 H) 2.13 (s, 3 H) 2.68-2.83 (m, 3 H) 3.01 (br t, J = 11.8 Hz, 1 H) 3.15-3.21 (m, 1 H) 3.58-3.71 (m, 1 H) 3.81-3.91 (m, 1 H) 3.98 (br d, J = 11.8 Hz, 1 H) 4.07 (dd, J = 11.8, 3.8 Hz, 1 H) 4.34-4.46 (m, 1 H) 7.07 (br s, 1 H) 7.12 (br s, 1 H) 7.26 (d, J = 9.5 Hz, 1 H) 7.42 (d, J = 9.5 Hz, 1 H) 10.21 (br s, 1 H) |
| 253 | 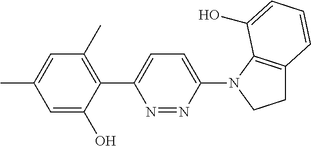 [SMILES: Cc1cc(C)c(-c2ccc(N3CCc4cccc(O)c43)nn2)c(O)c1] | 334 1.62 U | 1H NMR (400 MHz, DMSO-d6, 30° C.) δ ppm 2.06 (s, 3 H) 2.24 (s, 3 H) 3.21 (t, J = 8.5 Hz, 2 H) 4.09 (t, J = 8.5 Hz, 2 H) 6.60 (br s, 1 H) 6.61 (br s, 1 H) 6.67 (br d, J = 7.7 Hz, 1 H) 6.72 (br d, J = 7.7 Hz, 1 H) 6.90 (t, J = 7.7 Hz, 1 H) 7.40 (d, J = 9.5 Hz, 1 H) 7.58 (d, J = 9.5 Hz, 1 H) 9.40 (s, 1 H) 13.52 (s, 1 H) |
| 254 | 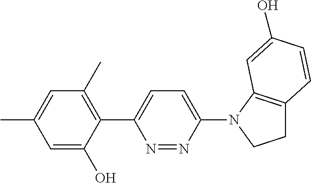 [SMILES: Cc1cc(C)c(-c2ccc(N3CCc4ccc(O)cc43)nn2)c(O)c1] | 334 1.28 U | 1H NMR (400 MHz, DMSO-d6, 30° C.) δ ppm 2.07 (s, 3 H) 2.24 (s, 3 H) 3.12 (t, J = 8.5 Hz, 2 H) 4.09 (t, J = 8.5 Hz, 2 H) 6.30 (dd, J = 8.0, 2.2 Hz, 1 H) 6.60 (br s, 1 H) 6.61 (br s, 1 H) 7.00 (d, J = 8.0 Hz, 1 H) 7.24 (d, J = 9.5 Hz, 1 H) 7.49 (d, J = 9.5 Hz, 1 H) 7.96 (d, J = 2.2 Hz, 1 H) 9.17 (br s, 1 H) 9.40 (br s, 1 H) |
| 255 | 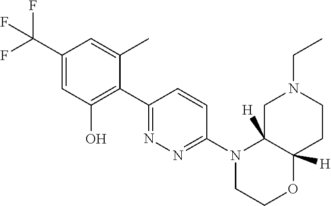 [SMILES: CCN1CC[C@@H]2OCCN(c3ccc(-c4c(C)cc(C(F)(F)F)cc4O)nn3)[C@@H]2C1] | 423 1.02 U | 1H NMR (400 MHz, DMSO-d6, 30° C.) δ ppm 0.98 (t, J = 7.3 Hz, 3 H) 1.78-1.91 (m, 2 H) 2.07-2.15 (m, 1 H) 2.13 (s, 3 H) 2.33-2.42 (m, 3 H) 2.56-2.65 (m, 2 H) 3.18 (td, J = 11.8, 3.8 Hz, 1 H) 3.63 (td, J = 11.8, 3.8 Hz, 1 H) 3.72-3.80 (m, 1 H) 3.96 (br d, J = 11.8 Hz, 1 H) 4.05 (dd, J = 11.8, 3.8 Hz, 1 H) 4.37-4.47 (m, 1 H) 7.07 (br s, 1 H) 7.12 (br s, 1 H) 7.24 (d, J = 9.5 Hz, 1 H) 7.40 (d, J = 9.5 Hz, 1 H) 10.13 (br s, 1 H) |
| 256 | 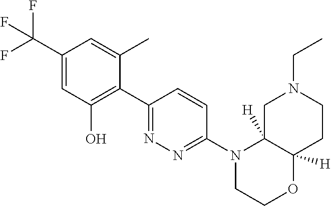 [SMILES: CCN1CC[C@H]2OCCN(c3ccc(-c4c(C)cc(C(F)(F)F)cc4O)nn3)[C@H]2C1] | 423 1.02 U | 1H NMR (400 MHz, DMSO-d6, 30° C.) δ ppm 0.98 (t, J = 7.3 Hz, 3 H) 1.78-1.91 (m, 2 H) 2.07-2.12 (m, 1 H) 2.13 (s, 3 H) 2.33-2.43 (m, 3 H) 2.57-2.66 (m, 2 H) 3.18 (td, J = 11.8, 3.8 Hz, 1 H) 3.63 (td, J = 11.8, 3.8 Hz, 1 H) 3.73-3.79 (m, 1 H) 3.96 (br d, J = 11.8 Hz, 1 H) 4.05 (dd, J = 11.8, 3.8 Hz, 1 H) 4.38-4.46 (m, 1 H) 7.07 (br s, 1 H) 7.12 (br s, 1 H) 7.24 (d, J = 9.5 Hz, 1 H) 7.40 (d, J = 9.5 Hz, 1 H) 10.13 (br s, 1 H) |
| 257 | 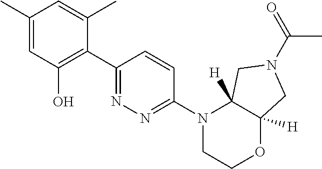 [SMILES: CC(=O)N1C[C@@H]2[C@@H](C1)OCCN2c1ccc(-c2c(C)cc(C)cc2O)nn1] | 367.3 2.55 E | 1H NMR (400 MHz, DMSO-d6) δ (ppm): 9.40 (s, 1H), 7.47-7.42 (m, 2H), 6.60 (s, 1H), 6.59 (s, 1H), 4.73-4.58 (m, 1H), 4.19-4.09 (m, 1H), 3.91 (m, 2H), 3.85-3.71 (m, 2H), 3.48 (t, J = 10.3 Hz, 0.5H), 3.42-3.35 (m, 1H), 3.27-3.16 (m, 0.5H), 3.14- 2.94 (m, 2H), 2.23 (s, 3H), 2.02 (d, J = 1.4 Hz, 3H), 1.97 (d, J = 1.5 Hz, 3H). |
| 258 | 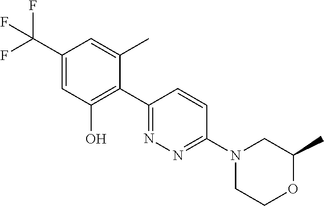 [SMILES: Cc1cc(C(F)(F)F)cc(O)c1-c1ccc(N2CCO[C@H](C)C2)nn1] | 354 1.38 U | 1H NMR (400 MHz, DMSO-d6, 30° C.) δ ppm 1.19 (d, J = 6.0 Hz, 3 H) 2.13 (s, 3 H) 2.64 (dd, J = 12.7, 10.5 Hz, 1 H) 2.96 (td, J = 12.7, 3.5 Hz, 1 H) 3.57- 3.67 (m, 2 H) 3.93-3.99 (m, 1 H) 4.17 (br d, J = 12.7 Hz, 1 H) 4.27 (br d, J = 12.7 Hz, 1 H) 7.06 (br s, 1 H) 7.11 (br s, 1 H) 7.32 (d, J = 9.5 Hz, 1 H) 7.40 (d, J = 9.5 Hz, 1 H) 10.00-10.30 (m, 1 H) |
| 259 | 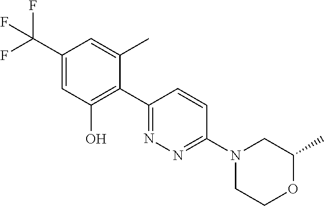 [SMILES: Cc1cc(C(F)(F)F)cc(O)c1-c1ccc(N2CCO[C@@H](C)C2)nn1] | 354 1.38 U | 1H NMR (400 MHz, DMSO-d6, 30° C.) δ ppm 1.19 (d, J = 6.0 Hz, 3 H) 2.13 (s, 3 H) 2.64 (dd, J = 12.7, 10.5 Hz, 1 H) 2.96 (td, J = 12.7, 3.5 Hz, 1 H) 3.57- 3.67 (m, 2 H) 3.93-4.01 (m, 1 H) 4.17 (br d, J = 12.7 Hz, 1 H) 4.27 (br d, J = 12.7 Hz, 1 H) 7.06 (br s, 1 H) 7.11 (br s, 1 H) 7.32 (d, J = 9.5 Hz, 1 H) 7.40 (d, J = 9.5 Hz, 1 H) 10.00-10.25 (m, 1 H) |
| 260 | 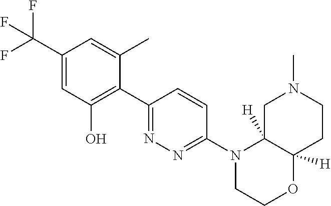 [SMILES: Cc1cc(C(F)(F)F)cc(O)c1-c1ccc(N2CCO[C@@H]3CCN(C)C[C@@H]32)nn1] | 409 0.99 U | 1H NMR (400 MHz, DMSO-d6, 30° C.) δ ppm 1.76- 1.93 (m, 2 H) 2.05-2.15 (m, 1 H) 2.13 (s, 3 H) 2.18 (s, 3 H) 2.29-2.38 (m, 1 H) 2.40-2.55 (m partially hidden, 2 H) 3.17 (td, J = 11.8, 3.8 Hz, 1 H) 3.63 (td, J = 11.8, 3.8 Hz, 1 H) 3.70-3.78 (m, 1 H) 3.95 (br d, J = 11.8 Hz, 1 H) 4.05 (dd, J = 11.8, 3.8 Hz, 1 H) 4.37-4.46 (m, 1 H) 7.06 (br s, 1 H) 7.12 (br s, 1 H) 7.25 (d, J = 9.5 Hz, 1 H) 7.40 (d, J = 9.5 Hz, 1 H) 10.14 (br s, 1 H) |
| 261 | 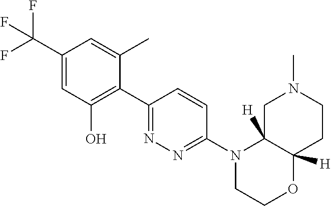 [SMILES: Cc1cc(C(F)(F)F)cc(O)c1-c1ccc(N2CCO[C@H]3CCN(C)C[C@H]32)nn1] | 409 0.99 U | 1H NMR (400 MHz, DMSO-d6, 30° C.) δ ppm 1.76- 1.93 (m, 2 H) 2.04-2.15 (m, 1 H) 2.13 (s, 3 H) 2.18 (s, 3 H) 2.29-2.37 (m, 1 H) 2.48-2.56 (m partially hidden, 2 H) 3.17 (td, J = 11.8, 3.8 Hz, 1 H) 3.63 (td, J = 11.8, 3.8 Hz, 1 H) 3.70-3.78 (m, 1 H) 3.95 (br d, J = 11.8 Hz, 1 H) 4.05 (dd, J = 11.8, 3.8 Hz, 1 H) 4.38-4.48 (m, 1 H) 7.06 (br s, 1 H) 7.12 (br s, 1 H) 7.25 (d, J = 9.5 Hz, 1 H) 7.40 (d, J = 9.5 Hz, 1 H) 10.15 (br s, 1 H) |
| 262 | 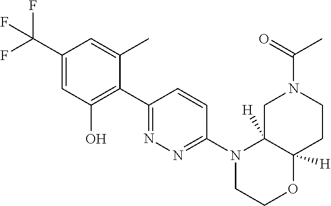 [SMILES: CC(=O)N1CC[C@H]2OCCN(c3ccc(-c4c(C)cc(C(F)(F)F)cc4O)nn3)[C@H]2C1] | 437 1.36 U | 1H NMR (400 MHz, DMSO-d6, 100° C.) δ ppm 1.79- 1.89 (m, 2 H) 2.02 (s, 3 H) 2.16 (s, 3 H) 2.72-4.57 (m, 4 H) 3.26 (td, J = 12.7, 3.8 Hz, 1 H) 3.71 (td, J = 11.8, 3.2 Hz, 1 H) 3.83-3.94 (m, 1 H) 4.01 (br d, J = 11.8 Hz, 1 H) 4.10 (dd, J = 11.8, 3.2 Hz, 1 H) 4.26-4.42 (m, 1 H) 7.06-7.09 (m, 2 H) 7.24 (br d, J = 9.5 Hz, 1 H) 7.41 (d, J = 9.5 Hz, 1 H) 9.65- 9.97 (m, 1 H) |
| 263 | 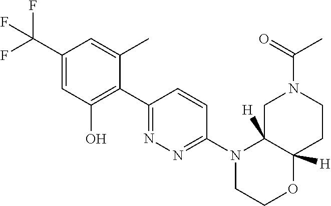 [SMILES: CC(=O)N1CC[C@@H]2OCCN(c3ccc(-c4c(C)cc(C(F)(F)F)cc4O)nn3)[C@@H]2C1] | 437 1.36 U | 1H NMR (400 MHz, DMSO-d6, 100° C.) δ ppm 1.80- 1.89 (m, 2 H) 2.02 (s, 3 H) 2.16 (s, 3 H) 2.73-4.46 (m, 4 H) 3.26 (td, J = 12.7, 3.8 Hz, 1 H) 3.71 (td, J = 11.8, 3.2 Hz, 1 H) 3.86-3.91 (m, 1 H) 4.01 (br d, J = 11.8 Hz, 1 H) 4.10 (dd, J = 11.8, 3.2 Hz, 1 H) 4.28-4.40 (m, 1 H) 7.06-7.08 (m, 2 H) 7.24 (br d, J = 9.5 Hz, 1 H) 7.41 (d, J = 9.5 Hz, 1 H) 9.70- 9.90 (m, 1 H) |
| 264 | 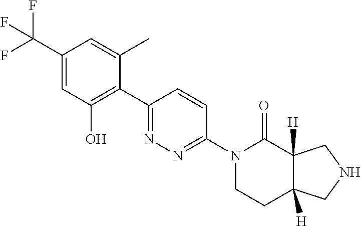 [SMILES: Cc1cc(C(F)(F)F)cc(O)c1-c1ccc(N2CC[C@H]3CNC[C@H]3C2=O)nn1] | 393 0.96 U | 1H NMR (400 MHz, DMSO-d6, 30° C.) δ ppm 1.76- 1.93 (m, 1 H) 2.11 (s, 3 H) 2.14-2.24 (m, 1 H) 2.88- 2.99 (m, 1 H) 2.99-3.10 (m, 1 H) 3.41-3.67 (m, 4 H) 3.89-3.96 (m partially hidden, 1 H) 4.46 (br dt, J = 13.0, 4.0 Hz, 1 H) 7.16 (br s, 1 H) 7.17 (br s, 1 H) 7.74 (d, J = 9.0 Hz, 1 H) 8.14 (d, J = 9.0 Hz, 1 H) 9.23-9.41 (m, 2 H) 10.33-10.52 (m, 1 H) |
| 265 | 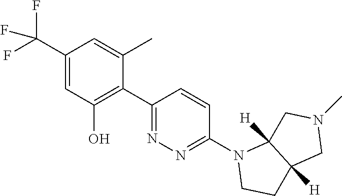 [SMILES: Cc1cc(C(F)(F)F)cc(O)c1-c1ccc(N2CC[C@H]3CN(C)C[C@H]32)nn1] | 379.4 2.68 E | 1H NMR (500 MHz, DMSO-d6) δ (ppm) : 10.13 (s, 1H), 7.34 (d, J = 9.2 Hz, 1H), 7.11 (s, 1H), 7.06 (d, J = 1.9 Hz, 1H), 6.96 (d, J = 9.3 Hz, 1H), 4.51- 4.36 (m, 1H), 3.63-3.47 (m, 2H), 2.94 (t, J = 4.5 Hz, 1H), 2.75-2.61 (m, 1H), 2.61-2.51 (m, 2H), 2.44-2.36 (m, 1H), 2.20 (s, 3H), 2.16-2.09 (m, 4H), 1.98-1.80 (m, 1H). |
| 266 | 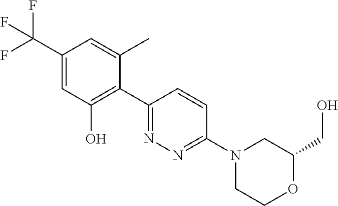 [SMILES: Cc1cc(C(F)(F)F)cc(O)c1-c1ccc(N2CCO[C@@H](CO)C2)nn1] | 370 1.20 U | 1H NMR (400 MHz, DMSO-d6, 30° C.) δ ppm 2.13 (s, 3 H) 2.74 (dd, J = 12.7, 10.0 Hz, 1 H) 3.00 (td, J = 12.7, 2.6 Hz, 1 H) 3.43-3.66 (m, 4 H) 3.96-4.03 (m, 1 H) 4.15 (br d, J = 12.7 Hz, 1 H) 4.34 (br d, J = 12.7 Hz, 1 H) 4.75-4.85 (m, 1 H) 7.06 (s, 1 H) 7.12 (s, 1 H) 7.31 (d, J = 9.5 Hz, 1 H) 7.41 (d, J = 9.5 Hz, 1 H) 10.05-10.25 (m, 1 H) |
| 267 | 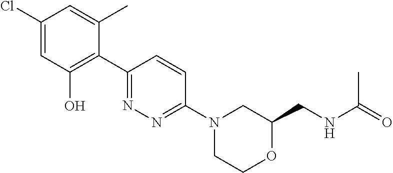 [SMILES: CC(=O)NC[C@@H]1CN(c2ccc(-c3c(C)cc(Cl)cc3O)nn2)CCO1] | 377 1.08 U | 1H NMR (400 MHz, DMSO-d6, 30° C.) δ ppm 1.85 (s, 3 H) 2.05 (s, 3 H) 2.69 (dd, J = 12.7, 10.5 Hz, 1 H) 2.99 (td, J = 12.7, 3.5 Hz, 1 H) 3.23 (t, J = 5.7 Hz, 2 H) 3.52-3.64 (m, 2 H) 4.00 (br d, J = 11.5 Hz, 1 H) 4.11 (br d, J = 12.7 Hz, 1 H) 4.29 (br d, J = 12.7 Hz, 1 H) 6.81 (d, J = 2.0 Hz, 1 H) 6.84 (d, J = 2.0 Hz, 1 H) 7.29 (d, J = 9.5 Hz, 1 H) 7.37 (d, J = 9.5 Hz, 1 H) 8.00 (t, J = 5.7 Hz, 1 H) 9.94 (s, 1 H) |
| 268 | 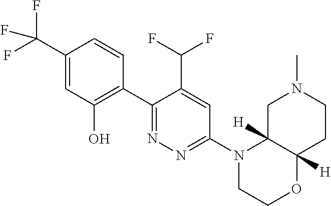 [SMILES: CN1CC[C@@H]2OCCN(c3cc(C(F)F)c(-c4ccc(C(F)(F)F)cc4O)nn3)[C@@H]2C1] | 445 1.04 U | 1H NMR (400 MHz, DMSO-d6, 30° C.) δ ppm 1.77- 1.95 (m, 2 H) 2.07-2.16 (m, 1 H) 2.19 (s, 3 H) 2.29- 2.40 (m, 1 H) 2.45-2.58 (m partially hidden, 2 H) 3.19-3.28 (m partially hidden, 1 H) 3.64 (td, J = 11.8, 3.8 Hz, 1 H) 3.71-3.78 (m, 1 H) 3.98-4.12 (m, 2 H) 4.41-4.66 (m, 1 H) 6.85 (t, J = 54.3 Hz, 1 H) 7.23 (br s, 1 H) 7.27 (br d, J = 7.8 Hz, 1 H) 7.35 (s, 1 H) 7.51 (d, J = 7.8 Hz, 1 H) 10.50-10.80 (m, 1 H) |
| 269 | 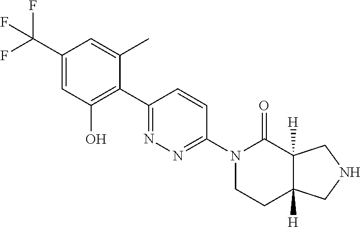 [SMILES: Cc1cc(C(F)(F)F)cc(O)c1-c1ccc(N2CC[C@H]3CNC[C@@H]3C2=O)nn1] | 393 0.95 U | 1H NMR (400 MHz, DMSO-d6, 30° C.) δ ppm 1.77- 1.92 (m, 1 H) 2.11 (s, 3 H) 2.14-2.24 (m, 1 H) 2.88- 2.99 (m, 1 H) 2.99-3.10 (m, 1 H) 3.41-3.67 (m, 4 H) 3.91-3.96 (m, 1 H) 4.46 (br dt, J = 13.0, 4.0 Hz, 1 H) 7.16 (br s, 1 H) 7.17 (br s, 1 H) 7.73 (d, J = 9.0 Hz, 1 H) 8.14 (d, J = 9.0 Hz, 1 H) 9.23-9.43 (m, 2 H) 10.32-10.52 (m, 1 H) |
| 270 | 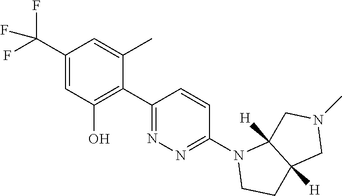 [SMILES: Cc1cc(C(F)(F)F)cc(O)c1-c1ccc(N2CC[C@H]3CN(C)C[C@H]32)nn1] | 379.3 2.67 E | 1H NMR (500 MHz, DMSO-d6) δ (ppm) : 10.22 (s, 1H), 7.34 (d, J = 9.3 Hz, 1H), 7.10 (s, 1H), 7.06 (s, 1H), 6.96 (d, J = 9.3 Hz, 1H), 4.56-4.31 (m, 1H), 3.77-3.58 (m, 1H), 3.58-3.43 (m, 1H), 3.09- 2.77 (m, 1H), 2.71-2.59 (m, 1H), 2.61-2.53 (m, 2H), 2.44-2.34 (m, 1H), 2.20 (s, 3H), 2.15-2.06 (m, 4H), 2.01-1.82 (m, 1H). |
| 271 | 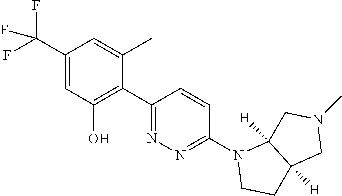 [SMILES: Cc1cc(C(F)(F)F)cc(O)c1-c1ccc(N2CC[C@@H]3CN(C)C[C@@H]32)nn1] | 379.4 2.66 E | 1H NMR (500 MHz, DMSO-d6) δ (ppm) : 10.14 (s, 1H), 7.34 (d, J = 9.3 Hz, 1H), 7.11 (s, 1H), 7.06 (s, 1H), 6.96 (d, J = 9.3 Hz, 1H), 4.62-4.26 (m, 1H), 3.67-3.62 (m, 1H), 3.57-3.50 (m, 1H), 3.00- 2.90 (m, 1H), 2.73-2.62 (m, 2H), 2.61-2.50 (m, 2H), 2.46-2.33 (m, 1H), 2.20 (s, 3H), 2.18-2.09 (m, 3H), 2.01-1.79 (m, 1H). |
| 272 | 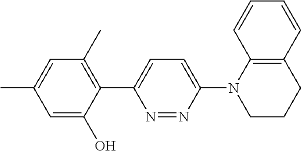 [SMILES: Cc1cc(C)c(-c2ccc(N3CCCc4ccccc43)nn2)c(O)c1] | 332 1.50 U | 1H NMR (400 MHz, DMSO-d6, 30° C.) δ ppm 1.96 (quin, J = 6.4 Hz, 2 H) 2.07 (s, 3 H) 2.23 (s, 3 H) 2.77 (t, J = 6.4 Hz, 2 H) 3.98 (t, J = 6.4 Hz, 2 H) 6.59 (br s, 1 H) 6.60 (br s, 1 H) 6.98 (br t, J = 7.5 Hz, 1 H) 7.13 (br t, J = 7.5 Hz, 1 H) 7.21 (br d, J = 7.5 Hz, 1 H) 7.25 (br d, J = 7.5 Hz, 1 H) 7.36 (d, J = 9.5 Hz, 1 H) 7.46 (d, J = 9.5 Hz, 1 H) 9.41 (s, 1 H) |
| 273 | 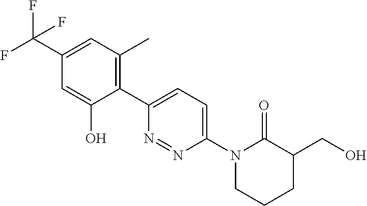 [SMILES: Cc1cc(C(F)(F)F)cc(O)c1-c1ccc(N2CCCC(CO)C2=O)nn1] | 382 1.36 U | 1H NMR (400 MHz, DMSO-d6, 30° C.) δ ppm 1.84- 2.09 (m, 4 H) 2.11 (s, 3 H) 2.61-2.70 (m, 1 H) 3.66- 3.84 (m, 2 H) 3.99-4.08 (m, 2 H) 4.70 (br t, J = 5.3 Hz, 1 H) 7.10 (br s, 1 H) 7.16 (br s, 1 H) 7.67 (d, J = 9.0 Hz, 1 H) 8.03 (d, J = 9.0 Hz, 1 H) 10.25-10.50 (m, 1 H) |
| 274 | 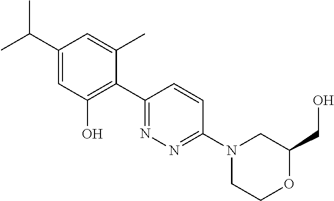 [SMILES: Cc1cc(C(C)C)cc(O)c1-c1ccc(N2CCO[C@H](CO)C2)nn1] | 344 1.12 U | 1H NMR (400 MHz, DMSO-d6, 30° C.) δ ppm 1.20 (d, J = 7.0 Hz, 6 H) 2.05 (s, 3 H) 2.71 (dd, J = 12.7, 10.0 Hz, 1 H) 2.75-2.83 (m, 1 H) 2.97 (br td, J = 11.6, 2.6 Hz, 1 H) 3.43-3.65 (m, 4 H) 3.96-4.02 (m, 1 H) 4.12 (br d, J = 12.7 Hz, 1 H) 4.31 (br d, J = 12.7 Hz, 1 H) 4.80 (t, J = 5.4 Hz, 1 H) 6.64 (br s, 2 H) 7.26 (d, J = 9.5 Hz, 1 H) 7.37 (d, J = 9.5 Hz, 1 H) 9.36 (s, 1 H) |
| 275 | 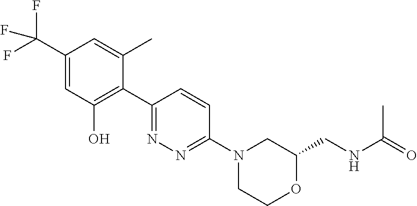 [SMILES: CC(=O)NC[C@H]1CN(c2ccc(-c3c(C)cc(C(F)(F)F)cc3O)nn2)CCO1] | 411.4 2.46 E | 1H NMR (400 MHz, DMSO-d6) δ (ppm): 10.24 (s, 1H), 8.06 (t, J = 5.9 Hz, 1H), 7.43 (d, J = 9.4 Hz, 1H), 7.33 (d, J = 9.5 Hz, 1H), 7.12 (s, 1H), 7.06 (s, 1H), 4.32 (d, J = 12.8 Hz, 1H), 4.13 (d, J = 12.8 Hz, 1H), 4.06-3.97 (m, 1H), 3.67-3.51 (m, 2H), 3.24 (t, J = 5.8 Hz, 2H), 3.00 (td, J = 12.4, 3.5 Hz, 1H), 2.75-2.64 (m, 1H), 2.13 (s, 3H), 1.85 (s, 3H). |
| 276 | 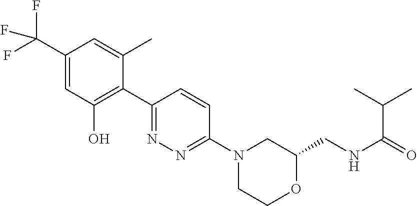 [SMILES: Cc1cc(C(F)(F)F)cc(O)c1-c1ccc(N2CCO[C@@H](CNC(=O)C(C)C)C2)nn1] | 439.4 2.69 E | 1H NMR (400 MHz, DMSO-d6) δ (ppm): 10.27 (s, 1H), 7.94 (t, J = 5.8 Hz, 1H), 7.43 (d, J = 9.4 Hz, 1H), 7.31 (d, J = 9.5 Hz, 1H), 7.11 (s, 1H), 7.08- 7.03 (m, 1H), 4.32 (d, J = 12.8 Hz, 1H), 4.13 (d, J = 12.8 Hz, 1H), 4.05-3.97 (m, 1H), 3.67-3.51 (m, 2H), 3.30-3.15 (m, 2H), 3.00 (td, J = 12.3, 3.5 Hz, 1H), 2.74-2.67 (m, 1H), 2.42 (h, J = 6.8 Hz, 1H), 2.13 (s, 3H), 1.02 (d, J = 6.8 Hz, 6H). |
| 277 | 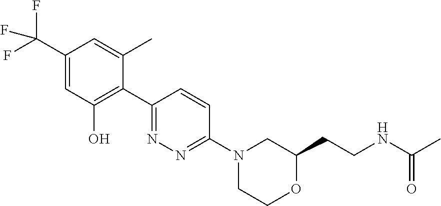 [SMILES: CC(=O)NCC[C@@H]1CN(c2ccc(-c3c(C)cc(C(F)(F)F)cc3O)nn2)CCO1] | 425 1.23 U | 1H NMR (400 MHz, DMSO-d6, 30° C.) δ ppm 1.59- 1.68 (m, 2 H) 1.80 (s, 3 H) 2.13 (s, 3 H) 2.69 (dd, J = 12.7, 10.5 Hz, 1 H) 2.98 (td, J = 12.7, 3.5 Hz, 1 H) 3.08-3.27 (m, 2 H) 3.50-3.56 (m, 1 H) 3.60 (td, J = 11.6, 2.5 Hz, 1 H) 3.99 (dd, J = 11.6, 2.5 Hz, 1 H) 4.19 (br d, J = 12.7 Hz, 1 H) 4.25 (br d, J = 12.7 Hz, 1 H) 7.06 (s, 1 H) 7.11 (s, 1 H) 7.34 (d, J = 9.5 Hz, 1 H) 7.41 (d, J = 9.5 Hz, 1 H) 7.85 (br t, J = 5.5 Hz, 1 H) 10.05-10.25 (m, 1 H) |
| 278 | 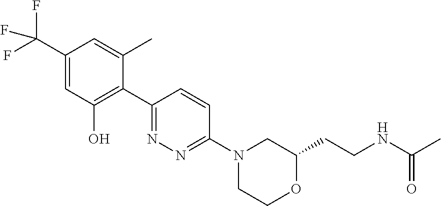 [SMILES: CC(=O)NCC[C@H]1CN(c2ccc(-c3c(C)cc(C(F)(F)F)cc3O)nn2)CCO1] | 425 1.23 U | 1H NMR (400 MHz, DMSO-d6, 30° C.) δ ppm 1.58- 1.69 (m, 2 H) 1.80 (s, 3 H) 2.13 (s, 3 H) 2.69 (dd, J = 12.7, 10.5 Hz, 1 H) 2.98 (td, J = 12.7, 3.5 Hz, 1 H) 3.07-3.27 (m, 2 H) 3.49-3.56 (m, 1 H) 3.60 (td, J = 11.6, 2.5 Hz, 1 H) 3.99 (dd, J = 11.6, 2.5 Hz, 1 H) 4.19 (br d, J = 12.7 Hz, 1 H) 4.25 (br d, J = 12.7 Hz, 1 H) 7.06 (s, 1 H) 7.11 (s, 1 H) 7.34 (d, J = 9.5 Hz, 1 H) 7.41 (d, J = 9.5 Hz, 1 H) 7.85 (t, J = 5.5 Hz, 1 H) 10.05-10.25 (m, 1 H) |
| 279 | 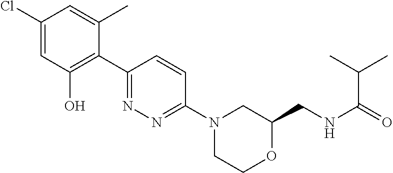 [SMILES: Cc1cc(Cl)cc(O)c1-c1ccc(N2CCO[C@H](CNC(=O)C(C)C)C2)nn1] | 405.4 2.60 E | ¹H NMR (500 MHz, DMSO-d₆) δ (ppm): 9.97 (s, 1H), 7.91 (s, 1H), 7.38 (d, J = 9.4 Hz, 1H), 7.27 (d, J = 9.4 Hz, 1H), 7.02-6.67 (m, 2H), 4.32-4.23 (m, 1H), 4.15-4.04 (m, 1H), 4.02-3.87 (m, 1H), 3.70-3.49 (m, 2H), 3.29-3.14 (m, 2H), 3.09- 2.89 (m, 1H), 2.78-2.58 (m, 1H), 2.46-2.33 (m, 1H), 2.04 (s, 3H), 1.01 (d, J = 6.8 Hz, 6H). |
| 280 | 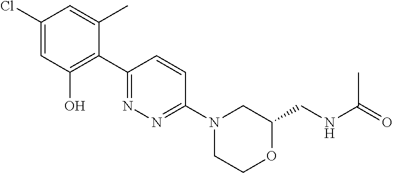 [SMILES: CC(=O)NC[C@H]1CN(c2ccc(-c3c(C)cc(Cl)cc3O)nn2)CCO1] | 377.3 2.47 E | ¹H NMR (500 MHz, DMSO-d₆) δ (ppm): 9.96 (s, 1H), 8.03 (s, 1H), 7.38 (d, J = 9.3 Hz, 1H), 7.29 (d, J = 9.4 Hz, 1H), 6.83 (dd, J = 15.7, 2.1 Hz, 2H), 4.36-4.20 (m, 1H), 4.14-4.07 (m, 1H), 4.06- 3.93 (m, 1H), 3.66-3.51 (m, 2H), 3.23 (t, J = 5.9 Hz, 2H), 3.10-2.85 (m, 1H), 2.75-2.63 (m, 1H), 2.05 (s, 3H), 1.85 (s, 3H). |
| 283 | 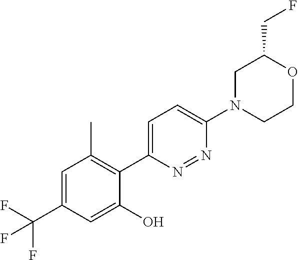 [SMILES: Cc1cc(C(F)(F)F)cc(O)c1-c1ccc(N2CCO[C@@H](CF)C2)nn1] | 372 1.38 B | ¹H NMR (400 MHz, DMSO-d6, 30° C.) δ ppm 2.13 (s, 3 H) 2.85 (dd, J = 12.6, 10.8 Hz, 1 H) 3.02 (td, J = 12.6, 3.0 Hz, 1 H) 3.66 (td, J = 11.5, 3.0 Hz, 1 H) 3.76-3.87 (m, 1 H) 4.04 (dd, J = 11.5, 3.0 Hz, 1 H) 4.16 (br d, J = 12.6 Hz, 1 H) 4.35 (br d, J = 12.6 Hz, 1 H) 4.45-4.65 (m, 2 H) 7.07 (br s, 1 H) 7.12 (br s, 1 H) 7.34 (d, J = 9.5 Hz, 1 H) 7.43 (d, J = 9.5 Hz, 1 H) 10.05-10.24 (m, 1 H) |
| 284 | 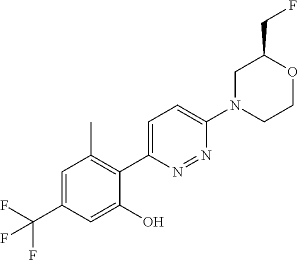 [SMILES: Cc1cc(C(F)(F)F)cc(O)c1-c1ccc(N2CCO[C@H](CF)C2)nn1] | 372 1.38 B | 1H NMR (400 MHz, DMSO-d₆, 30° C.) δ ppm 2.13 (s, 3 H) 2.85 (dd, J = 12.6, 10.8 Hz, 1 H) 3.02 (td, J = 12.6, 3.0 Hz, 1 H) 3.66 (td, J = 11.5, 3.0 Hz, 1 H) 3.76-3.87 (m, 1 H) 4.04 (dd, J = 11.5, 3.0 Hz, 1 H) 4.16 (br d, J = 12.6 Hz, 1 H) 4.35 (br d, J = 12.6 Hz, 1 H) 4.45-4.65 (m, 2 H) 7.06 (br s, 1 H) 7.12 (br s, 1 H) 7.34 (d, J = 9.5 Hz, 1 H) 7.43 (d, J = 9.5 Hz, 1 H) 10.08-10.20 (m, 1 H) |
| 285 | 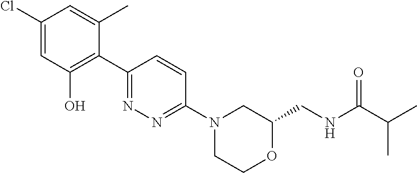 [SMILES: Cc1cc(Cl)cc(O)c1-c1ccc(N2CCO[C@@H](CNC(=O)C(C)C)C2)nn1] | 405.4 2.60 E | ¹H NMR (500 MHz, DMSO-d₆) δ (ppm): 10.02 (s, 1H), 7.92 (t, J = 5.8 Hz, 1H), 7.38 (d, J = 9.4 Hz, 1H), 7.28 (d, J = 9.5 Hz, 1H), 6.86-6.79 (m, 2H), 4.29 (d, J = 12.7 Hz, 1H), 4.11 (d, J = 12.8 Hz, 1H), 4.04-3.98 (m, 1H), 3.66-3.58 (m, 1H), 3.57- 3.51 (m, 1H), 3.29-3.16 (m, 2H), 3.04-2.95 (m, 1H), 2.73-2.65 (m, 1H), 2.47-2.39 (m, 1H), 2.05 (s, 3H), 1.02 (d, J = 6.8 Hz, 6H) |
| 286 | 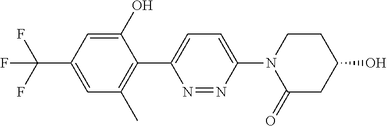 [SMILES: Cc1cc(C(F)(F)F)cc(O)c1-c1ccc(N2CC[C@H](O)CC2=O)nn1] | 368 1.26 B | ¹H NMR (400 MHz, DMSO-d₆, 30° C.) δ ppm 1.86- 1.95 (m, 1 H) 2.08-2.16 (m, 1 H) 2.11 (s, 3 H) 2.43- 2.48 (m partially hidden, 1 H) 2.81 (dd, J = 17.3, 4.8 Hz, 1 H) 3.91-4.00 (m, 1 H) 4.14-4.24 (m, 2 H) 5.14 (d, J = 3.5 Hz, 1 H) 7.10 (br s, 1 H) 7.16 (br s, 1 H) 7.67 (d, J = 9.0 Hz, 1 H) 8.03 (d, J = 9.0 Hz, 1 H) 10.18-10.40 (m, 1 H) |
| 287 | 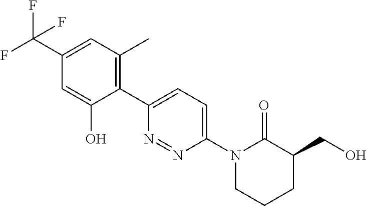 [SMILES: Cc1cc(C(F)(F)F)cc(O)c1-c1ccc(N2CCC[C@H](CO)C2=O)nn1] | 382 1.35 B | 1H NMR (400 MHz, DMSO-d6, 30° C.) δ ppm 1.82- 1.97 (m, 2 H), 1.97-2.09 (m, 2 H), 2.11 (s, 3 H), 2.60-2.77 (m, 1 H), 3.63-3.75 (m, 1 H), 3.75- 3.87 (m, 1 H), 3.95-4.15 (m, 2 H), 4.62-4.77 (m, 1 H), 7.10 (br s, 1 H), 7.16 (br s, 1 H), 7.67 (d, J = 9.1 Hz, 1 H), 8.03 (d, J = 9.1 Hz, 1 H), 10.04- 10.55 (m, 1 H) |
| 288 | 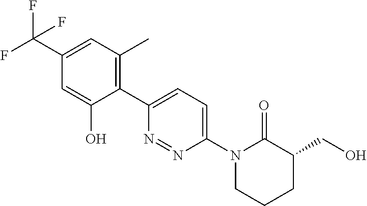 [SMILES: Cc1cc(C(F)(F)F)cc(O)c1-c1ccc(N2CCC[C@@H](CO)C2=O)nn1] | 382 1.35 B | 1H NMR (400 MHz, DMSO-d6, 30° C.) δ ppm 1.82- 1.97 (m, 2 H), 1.97-2.09 (m, 2 H), 2.11 (s, 3 H), 2.60-2.77 (m, 1 H), 3.63-3.75 (m, 1 H), 3.75- 3.87 (m, 1 H), 3.95-4.15 (m, 2 H), 4.62-4.77 (m, 1 H), 7.10 (br s, 1 H), 7.16 (br s, 1 H), 7.67 (d, J = 9.1 Hz, 1 H), 8.03 (d, J = 9.1 Hz, 1 H), 10.00 - 10.62 (m, 1 H) |
| 289 | 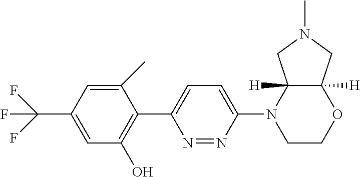 [SMILES: Cc1cc(C(F)(F)F)cc(O)c1-c1ccc(N2CCO[C@@H]3CN(C)C[C@H]32)nn1] | 394.2 1.09 P | 1H NMR (500 MHz, DMSO-d6) δ (ppm): 10.26 (s, 1H), 7.47 (d, J = 9.2 Hz, 1H), 7.40 (d, J = 9.4 Hz, 1H), 7.12 (s, 1H), 7.07 (s, 1H), 4.09 (d, J = 11.7 Hz, 1H), 3.93 (d, J = 12.5 Hz, 1H), 3.84 (t, J = 11.7 Hz, 1H), 3.80-3.72 (m, 1H), 3.68 (t, J = 8.3 Hz, 1H), 3.27-3.22 (m, 1H), 3.01 (t, J = 11.9 Hz, 1H), 2.90 (t, J = 7.9 Hz, 1H), 2.82 (t, J = 9.9 Hz, 1H), 2.71- 2.65 (m, 1H), 2.41 (s, 3H), 2.11 (s, 3H) |
| 290 | 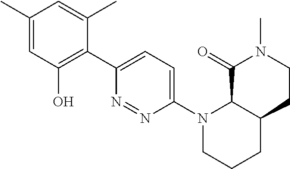 [SMILES: Cc1cc(C)c(-c2ccc(N3CCC[C@@H]4CCN(C)C(=O)[C@@H]43)nn2)c(O)c1] | 367 1.04 U | 1H NMR (400 MHz, DMSO-d₆, 80° C.) δ ppm 1.35- 1.44 (m, 1 H) 1.49-1.61 (m, 1 H) 1.68-1.81 (m, 3 H) 2.08 (s, 3 H) 2.17-2.27 (m, 2 H) 2.22 (s, 3 H) 2.61-2.72 (m, 1 H) 2.82 (s, 3 H) 3.28-3.41 (m, 2 H) 4.30 (br d, J = 11.8 Hz, 1 H) 5.28 (br d, J = 4.5 Hz, 1 H) 6.56 (br s, 1 H) 6.58 (br s, 1 H) 7.15 (d, J = 9.5 Hz, 1 H) 7.27 (d, J = 9.5 Hz, 1 H) 8.94 - 9.81 (m, 1 H) |
| 291 | 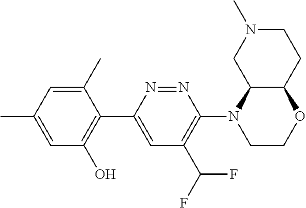 [SMILES: Cc1cc(C)c(-c2cc(C(F)F)c(N3CCO[C@@H]4CCN(C)C[C@@H]43)nn2)c(O)c1] | 405 1.02 U | 1H NMR (500 MHz, DMSO-d6, 27°° C.) d ppm 1.73- 1.86 (m, 2 H), 2.05 (s, 3 H), 2.07-2.13 (m, 1 H), 2.17 (br s, 3 H), 2.24 (s, 3 H), 2.44-2.49 (m partially hidden, 1 H), 2.51-2.53 (m partially hidden, 1 H), 2.55-2.62 (m, 1 H), 3.27-3.31 (m partially hidden, 1 H), 3.60-3.78 (m, 2 H), 3.80- 3.86 (m, 1 H), 3.89-3.94 (m, 1 H), 3.96-4.01 (m, 1 H), 6.62 (br s, 2 H), 7.20 (t, J = 53.4 Hz, 1 H), 7.64 (s, 1 H), 9.46 (s, 1 H) |
| 292 | 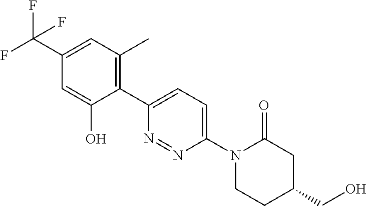 [SMILES: Cc1cc(C(F)(F)F)cc(O)c1-c1ccc(N2CC[C@@H](CO)CC2=O)nn1] | 382 1.27 B | 1H NMR (400 MHz, DMSO-d₆, 30° C.) δ ppm 1.59- 1.71 (m, 1 H) 2.04-2.18 (m, 2 H) 2.11 (s, 3 H) 2.30- 2.39 (m, 1 H) 2.54-2.62 (m, 1 H) 3.34-3.46 (m, 2 H) 3.95-4.05 (m, 1 H) 4.11-4.19 (m, 1 H) 4.65- 4.77 (m, 1 H) 7.10 (br s, 1 H) 7.16 (br s, 1 H) 7.67 (d, J = 9.2 Hz, 1 H) 8.06 (d, J = 9.2 Hz, 1 H) 10.17- 10.42 (m, 1 H) |
| 293 | 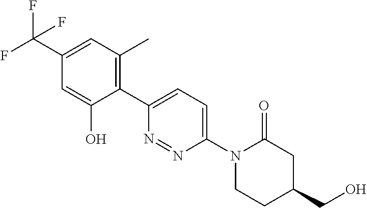 [SMILES: Cc1cc(C(F)(F)F)cc(O)c1-c1ccc(N2CC[C@H](CO)CC2=O)nn1] | 382 1.27 B | 1H NMR (400 MHz, DMSO-d₆, 30° C.) δ ppm 1.59- 1.71 (m, 1 H) 2.06-2.18 (m, 2 H) 2.11 (s, 3 H) 2.30- 2.40 (m, 1 H) 2.53-2.62 (m, 1 H) 3.34-3.46 (m, 2 H) 3.95-4.05 (m, 1 H) 4.11-4.19 (m, 1 H) 4.66- 4.76 (m, 1 H) 7.10 (br s, 1 H) 7.15 (br s, 1 H) 7.67 (d, J = 9.2 Hz, 1 H) 8.06 (d, J = 9.2 Hz, 1 H) 10.09- 10.40 (m, 1 H) |
| 295 | 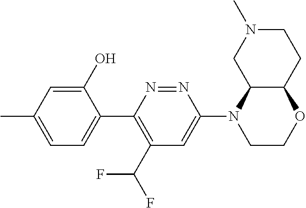 [SMILES: Cc1ccc(-c2nnc(N3CCO[C@@H]4CCN(C)C[C@@H]43)cc2C(F)F)c(O)c1] | 391 0.93 U | 1H NMR (400 MHz, DMSO-d6, 30° C.) d ppm 1.75- 1.84 (m, 1 H), 1.84-1.95 (m, 1 H), 2.08-2.16 (m, 1 H), 2.19 (br s, 3 H), 2.30 (s, 3 H), 2.31-2.40 (m, 1 H), 2.52-2.58 (m partially hidden, 2 H), 3.21 (br td, J = 3.8 and 12.7 Hz, 1 H), 3.63 (td, J = 3.2 and 11.8 Hz, 1 H), 3.72-3.77 (m, 1 H), 3.99 (br d, J = 12.7 Hz, 1 H), 4.05 (dd, J = 3.8 and 11.8 Hz, 1 H), 4.48- 4.57 (m, 1 H), 6.77 (t, J=54.8 Hz, 1 H), 6.74-6.78 (m, 1 H), 6.77 (s, 1 H), 7.15 (d, J = 7.5 Hz, 1 H), 7.31 (br s, 1 H), 9.78 (s, 1 H) |
| 296 | 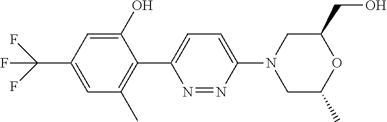 [SMILES: Cc1cc(C(F)(F)F)cc(O)c1-c1ccc(N2C[C@@H](CO)O[C@H](C)C2)nn1] | 384 1.20 B | 1H NMR (400 MHz, DMSO-d6, 100° C.) δ ppm 1.20 (d, J = 6.4 Hz, 3 H) 2.16 (s, 3 H) 3.28 (dd, J = 12.8, 7.3 Hz, 1 H) 3.49-3.64 (m, 3 H) 3.74 (dd, J = 12.8, 3.6 Hz, 1 H) 3.82 (dd, J = 12.8, 3.6 Hz, 1 H) 3.86-3.95 (m, 1 H) 4.01-4.13 (m, 1 H) 4.16-4.48 (m, 1 H) 7.06 (s, 2 H) 7.19 (d, J = 9.4 Hz, 1 H) 7.36 (d, J = 9.4 Hz, 1 H) 9.66-9.90 (m, 1 H) |
| 297 | 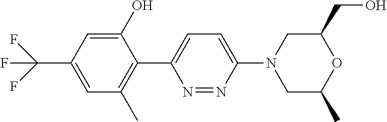 [SMILES: Cc1cc(C(F)(F)F)cc(O)c1-c1ccc(N2C[C@@H](CO)O[C@@H](C)C2)nn1] | 384 1.24 B | 1H NMR (400 MHz, DMSO-d6, 30° C.) δ ppm 1.20 (d, J = 6.1 Hz, 3 H) 2.13 (s, 3 H) 2.53-2.69 (m, 2 H) 3.41-3.77 (m, 4 H) 4.27 (br d, J = 12.4 Hz, 1 H) 4.38 (br d, J = 12.6 Hz, 1 H) 4.80 (br t, J = 5.3 Hz, 1 H) 7.06 (s, 1 H) 7.12 (s, 1 H) 7.31 (d, J = 9.4 Hz, 1 H) 7.40 (d, J = 9.4 Hz, 1 H) 10.13 (s, 1 H) |
| 298 | 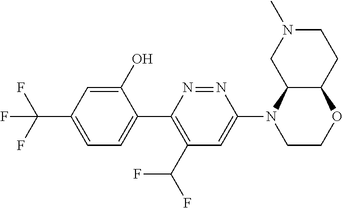 [SMILES: CN1CC[C@H]2OCCN(c3cc(C(F)F)c(-c4ccc(C(F)(F)F)cc4O)nn3)[C@H]2C1] | 445 1.03 U | 1H NMR (400 MHz, DMSO-d6, 30°° C.) δ ppm 1.71- 1.99 (m, 2 H), 2.05-2.15 (m, 1 H), 2.19 (s, 3 H), 2.31-2.42 (m, 1 H), 2.43-2.62 (m hidden, 2 H), 3.13-3.33 (m partially hidden, 1 H), 3.64 (td, J=11.9, 3.3 Hz, 1 H), 3.75 (q, J = 2.8 Hz, 1 H), 3.96- 4.10 (m, 2 H), 4.48-4.60 (m, 1 H), 6.85 (t, J = 54.3 Hz, 1 H), 7.23 (s, 1 H), 7.27 (br d, J = 7.8 Hz, 1 H), 7.35 (s, 1 H), 7.51 (d, J = 7.8 Hz, 1 H), 10.34-11.03 (m, 1 H) |
| 299 | 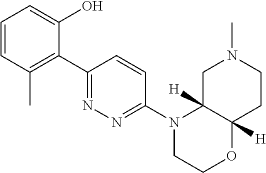 [SMILES: Cc1cccc(O)c1-c1ccc(N2CCO[C@H]3CCN(C)C[C@H]32)nn1] | 341 0.73 U | 1H NMR (400 MHz, DMSO-d6, 30° C.) δ ppm 1.73- 1.97 (m, 2 H), 2.06 (s, 3 H), 2.04-2.15 (m, 1 H), 2.18 (s, 3 H), 2.28-2.39 (m, 1 H), 2.44-2.58 (m, hidden, 2 H), 3.16 (td, J = 12.6, 3.9 Hz, 1 H), 3.63 (td, J = 11.8, 3.1 Hz, 1 H), 3.74 (q, J = 2.8 Hz, 1 H), 3.90-3.96 (m, 1 H), 4.05 (dd, J = 11.8, 3.5 Hz, 1 H), 4.35-4.48 (m, 1 H), 6.70-6.81 (m, 2 H), 7.10 (t, J = 7.8 Hz, 1 H), 7.21 (d, J = 9.4 Hz, 1 H), 7.36 (d, J = 9.4 Hz, 1 H), 9.42 (s, 1 H) |
| 300 | 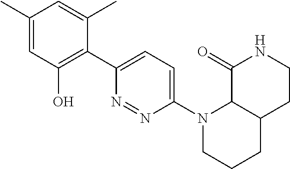 [SMILES: Cc1cc(C)c(-c2ccc(N3CCCC4CCNC(=O)C43)nn2)c(O)c1] | 353 1.00 U | 1H NMR (400 MHz, DMSO-d6, 80° C.) δ ppm 1.37- 1.50 (m, 1 H) 1.50-1.62 (m, 1 H) 1.68-1.77 (m, 2 H) 1.77-1.85 (m, 1 H) 2.10 (s, 3 H) 2.11-2.27 (m, 2 H) 2.24 (s, 3 H) 2.75 (td, J = 12.6, 2.8 Hz, 1 H) 3.17-3.34 (m, 2 H) 4.33 (br d, J = 12.6 Hz, 1 H) 5.26 (br d, J = 5.1 Hz, 1 H) 6.58 (br s, 1 H) 6.60 (br s, 1 H) 7.18 (d, J = 9.5 Hz, 1 H) 7.26 (br s, 1 H) 7.29 (d, J = 9.5 Hz, 1 H) 9.15-9.59 (m, 1 H) |
| 301 | 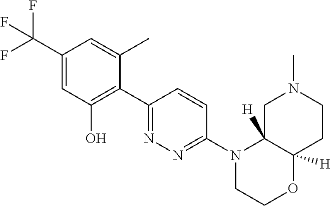 [SMILES: Cc1cc(C(F)(F)F)cc(O)c1-c1ccc(N2CCO[C@@H]3CCN(C)C[C@H]32)nn1] | 409.5 2.61 E | 1H NMR (500 MHz, DMSO-d₆) δ (ppm) : 10.23 (s, 1H), 7.50 (d, J = 9.1 Hz, 1H), 7.38 (d, J = 9.2 Hz, 1H), 7.13 (s, 1H), 7.08 (s, 1H), 3.93 (dt, J = 11.0, 4.4 Hz, 1H), 3.86 (ddd, J = 11.1, 8.3, 3.3 Hz, 1H), 3.68 (dt, J = 13.3, 4.1 Hz, 1H), 3.55-3.48 (m, 1H), 3.48-3.39 (m, 3H), 2.83 (d, J = 11.4 Hz, 1H), 2.17 (s, 3H), 2.12 (s, 3H), 2.10-2.02 (m, 1H), 1.89- 1.80 (m, 1H), 1.75 (t, J = 9.7 Hz, 1H), 1.64 (qd, J = 12.1, 4.3 Hz, 1H). |
| 302 | 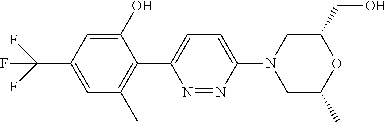 [SMILES: Cc1cc(C(F)(F)F)cc(O)c1-c1ccc(N2C[C@H](CO)O[C@H](C)C2)nn1] | 384 1.25 B | 1H NMR (400 MHz, DMSO-d6, 30° C.) 8 ppm 1.20 (d, J = 6.3 Hz, 3 H) 2.13 (s, 3 H) 2.52-2.68 (m, 2 H) 3.39-3.62 (m, 3 H) 3.63-3.74 (m, 1 H) 4.27 (br d, J = 12.1 Hz, 1 H) 4.38 (br d, J = 12.5 Hz, 1 H) 4.80 (t, J = 5.7 Hz, 1 H) 7.06 (s, 1 H) 7.12 (s, 1 H) 7.31 (d, J = 9.4 Hz, 1 H) 7.40 (d, J = 9.4 Hz, 1 H) 10.13 (s, 1 H) |
| 303 | 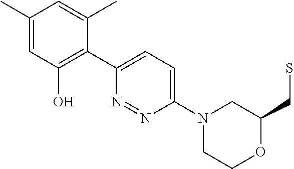 [SMILES: Cc1cc(C)c(-c2ccc(N3CCO[C@H](CS)C3)nn2)c(O)c1] | 332 1.12 U | 1H NMR (400 MHz, DMSO-d₆, 80° C.) δ ppm 2.08 (s, 3 H) 2.24 (s, 3 H) 2.31 (br t, J = 8.1 Hz, 1 H) 2.70 (dd, J = 8.1, 5.9 Hz, 2 H) 2.94 (dd, J = 13.1, 10.1 Hz, 1 H) 3.14 (ddd, J = 13.1, 11.3, 3.6 Hz, 1 H) 3.57- 3.71 (m, 2 H) 4.02 (ddd, J = 11.3, 3.6, 2.0 Hz, 1 H) 4.10 (d, J = 13.1 Hz, 1 H) 4.35 (br d, J = 13.1 Hz, 1 H) 6.62 (br s, 1 H) 6.63 (br s, 1 H) 7.57 (d, J = 9.5 Hz, 1 H) 7.64 (d, J = 9.5 Hz, 1 H) |
| 304 | 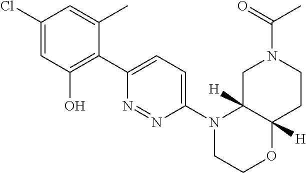 [SMILES: CC(=O)N1CC[C@@H]2OCCN(c3ccc(-c4c(C)cc(Cl)cc4O)nn3)[C@@H]2C1] | 403 1.19 U | 1H NMR (400 MHz, DMSO-d₆, 100° C.) δ ppm 1.79- 1.89 (m, 2 H) 2.01 (s, 3 H) 2.08 (s, 3 H) 2.73- 3.16 (m, 4 H) 3.25 (br td, J = 12.6, 3.8 Hz, 1 H) 3.70 (br td, J = 11.8, 3.2 Hz, 1 H) 3.83-3.92 (m, 1 H) 3.99 (br d, J = 12.6 Hz, 1 H) 4.09 (br dd, J = 11.8, 3.8 Hz, 1 H) 4.26-4.39 (m, 1 H) 6.79-6.83 (m, 2 H) 7.21 (br d, J = 9.5 Hz, 1 H) 7.37 (d, J = 9.5 Hz, 1 H) 9.36-9.72 (m, 1 H) |
| 305 | 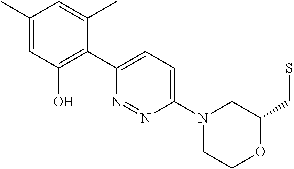 [SMILES: Cc1cc(C)c(-c2ccc(N3CCO[C@@H](CS)C3)nn2)c(O)c1] | 332 1.11 U | 1H NMR (400 MHz, DMSO-d₆, 80° C.) δ ppm 2.07 (s, 3 H) 2.24 (s, 3 H) 2.31 (br t, J = 8.1 Hz, 1 H) 2.70 (dd, J = 8.1, 5.9 Hz, 2 H) 2.89 (dd, J = 13.1, 10.1 Hz, 1 H) 3.10 (ddd, J = 13.1, 11.3, 3.6 Hz, 1 H) 3.56- 3.70 (m, 2 H) 4.01 (ddd, J = 11.3, 3.6, 2.0 Hz, 1 H) 4.09 (br d, J = 13.1 Hz, 1 H) 4.35 (br d, J = 13.1 Hz, 1 H) 6.60 (br s, 1 H) 6.61 (br s, 1 H) 7.42 (d, J = 9.5 Hz, 1 H) 7.51 (d, J = 9.5 Hz, 1 H) |
| 306 | 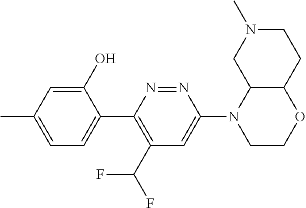 [SMILES: Cc1ccc(-c2nnc(N3CCOC4CCN(C)CC43)cc2C(F)F)c(O)c1] | 391 0.92 U | 1H NMR (400 MHz, DMSO-d6, 30° C.) δ ppm 1.76- 1.83 (m, 1 H), 1.85-1.96 (m, 1 H), 2.11 (br t, J = 11.1 Hz, 1 H), 2.18 (s, 3 H), 2.30 (s, 3 H), 2.31- 2.41 (m, 1 H), 2.44-2.57 (m hidden, 2 H), 3.21 (td, J = 12.7, 3.9 Hz, 1 H), 3.63 (td, J = 11.8, 3.1 Hz, 1 H), 3.74 (br q, J = 2.6 Hz, 1 H), 3.99 (br d, J = 12.7 Hz, 1 H), 4.05 (dd, J = 11.8, 3.5 Hz, 1 H), 4.46-4.58 (m, 1 H), 6.59-6.99 (m, 3 H), 7.15 (d, J = 7.5 Hz, 1 H), 7.31 (s, 1 H), 9.79 (s, 1 H) |
| 307 | 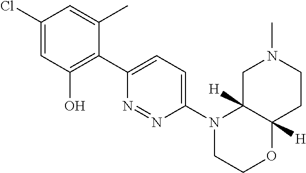 [SMILES: Cc1cc(Cl)cc(O)c1-c1ccc(N2CCO[C@H]3CCN(C)C[C@H]32)nn1] | 375 0.89 U | 1H NMR (400 MHz, DMSO-d6, 80° C.) δ ppm 1.74- 1.83 (m, 1 H) 1.83-1.94 (m, 1 H) 1.95-2.06 (m, 1 H) 2.07 (s, 3 H) 2.08-2.17 (m, 1 H) 2.19 (s, 3 H) 2.31-2.42 (m, 1 H) 2.50-2.61 (m partially hidden, 1 H) 3.19 (td, J = 12.8, 4.0 Hz, 2 H) 3.64 (td, J = 11.6, 3.3 Hz, 1 H) 3.74 (q, J = 2.8 Hz, 1 H) 3.93 (dd, J = 12.8, 2.6 Hz, 1 H) 4.04 (dd, J = 11.6, 3.5 Hz, 1 H) 4.31-4.44 (m, 1 H) 6.81 (s, 1 H) 7.17 (d, J = 9.4 Hz, 1 H) 7.33 (d, J = 9.4 Hz, 1 H) 9.55-9.85 (m, 1 H) |
| 308 | 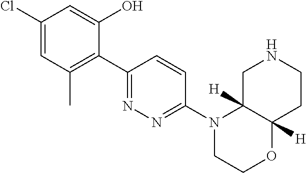 [SMILES: Cc1cc(Cl)cc(O)c1-c1ccc(N2CCO[C@H]3CCNC[C@H]32)nn1] | 361 0.88 U | 1H NMR (400 MHz, DMSO-d6, 30° C.) δ ppm 1.70- 1.83 (m, 2 H), 2.05 (s, 3 H), 2.57-2.70 (m, 3 H), 2.92 (br t, J = 11.4 Hz, 1 H), 3.05-3.20 (m, 1 H), 3.62 (td, J = 11.8, 3.1 Hz, 1 H), 3.83 (br q, J = 2.4 Hz, 1 H), 3.98 (br d, J = 12.5 Hz, 1 H), 4.06 (dd, J = 11.8, 3.4 Hz, 1 H), 4.18-4.29 (m, 1 H), 6.80-6.82 (m, 1 H), 6.83-6.84 (m, 1 H), 7.21 (d, J = 9.5 Hz, 1 H), 7.34 (d, J = 9.5 Hz, 1 H), 9.03-10.74 (m, 1 H) |
| 309 | 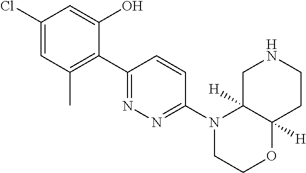 [SMILES: Cc1cc(Cl)cc(O)c1-c1ccc(N2CCO[C@@H]3CCNC[C@@H]32)nn1] | 361 0.88 U | 1H NMR (400 MHz, DMSO-d6, 30° C.) 8 ppm 1.72- 1.84 (m, 2 H), 2.05 (s, 3 H), 2.58-2.73 (m, 3 H), 2.92 (br t, J = 10.8 Hz, 1 H), 3.07-3.22 (m, 1 H), 3.62 (br t, J = 10.6 Hz, 1 H), 3.79-3.88 (m, 1 H), 3.98 (br d, J = 12.8 Hz, 1 H), 4.06 (br dd, J = 10.8, 2.6 Hz, 1 H), 4.17-4.31 (m, 1 H), 6.80-6.82 (m, 1 H), 6.83-6.85 (m, 1 H), 7.21 (br d, J = 9.1 Hz, 1 H), 7.35 (br d, J = 9.1 Hz, 1 H), 9.12-10.57 (m, 1 H) |
| 310 | 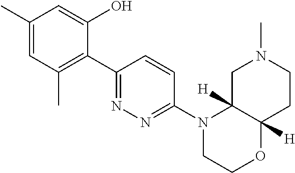 [SMILES: Cc1cc(C)c(-c2ccc(N3CCO[C@H]4CCN(C)C[C@H]43)nn2)c(O)c1] | 355 1.11 Z1 | 1H NMR (400 MHz, DMSO-d6, 30° C.) δ ppm 1.72- 1.83 (m, 1 H), 1.83-1.95 (m, 1 H), 2.03 (s, 3 H), 2.05-2.14 (m, 1 H), 2.18 (s, 3 H), 2.22 (s, 3 H), 2.28-2.38 (m, 1 H), 2.46-2.57 (m hidden, 2 H), 3.15 (td, J = 12.6, 3.9 Hz, 1 H), 3.63 (td, J = 11.4, 3.2 Hz, 1 H), 3.74 (br q, J = 2.6 Hz, 1 H), 3.92 (br dd, J = 12.8, 2.0 Hz, 1 H), 4.05 (dd, J = 11.4, 3.4 Hz, 1 H), 4.36-4.43 (m, 1 H), 6.56-6.58 (m, 1 H), 6.58- 6.59 (m, 1 H), 7.20 (d, J = 9.5 Hz, 1 H), 7.33 (d, J = 9.5 Hz, 1 H), 9.36 (s, 1 H) |
| 311 | 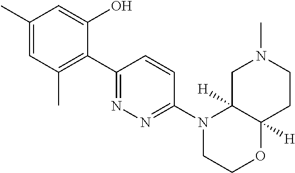 [SMILES: Cc1cc(C)c(-c2ccc(N3CCO[C@@H]4CCN(C)C[C@@H]43)nn2)c(O)c1] | 355 0.81 U | 1H NMR (400 MHz, DMSO-d6, 30° C.) d ppm 1.75- 1.93 (m, 2 H), 2.03 (s, 3 H), 2.04-2.14 (m, 1 H), 2.18 (s, 3 H), 2.22 (s, 3 H), 2.28-2.37 (m, 1 H), 2.51-2.56 (m hidden, 2 H), 3.16 (td, J = 3.8 and 12.6 Hz, 1 H), 3.63 (td, J = 3.2 and 11.8 Hz, 1 H), 3.74 (q, J = 2.8 Hz, 1 H), 3.92 (br d, J = 12.6 Hz, 1 H), 4.05 (dd, J = 3.6 and 11.8 Hz, 1 H), 4.36-4.44 (m, 1 H), 6.57 (br s, 1 H), 6.58 (br s, 1 H), 7.20 (d, J = 9.5 Hz, 1 H), 7.34 (d, J = 9.5 Hz, 1 H), 9.36 (s, 1 H) |
| 312 | 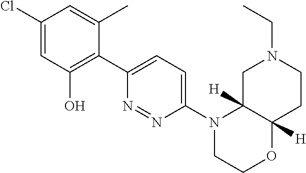 [SMILES: CCN1CC[C@@H]2OCCN(c3ccc(-c4c(C)cc(Cl)cc4O)nn3)[C@@H]2C1] | 389 0.92 U | 1H NMR (400 MHz, DMSO-d₆, 30° C.) δ ppm 0.97 (t, J = 7.3 Hz, 3 H) 1.76-1.90 (m, 2 H) 2.02-2.13 (m, 1 H) 2.05 (s, 3 H) 2.31-2.42 (m, 3 H) 2.55- 2.65 (m, 2 H) 3.17 (td, J=12.7, 3.8 Hz, 1 H) 3.62 (td, J=11.8, 3.3 Hz, 1 H) 3.75 (br q, J=2.8 Hz, 1 H) 3.94 (br d, J=12.7 Hz, 1 H) 4.05 (dd, J=11.8, 3.3 Hz, 1 H) 4.36 - 4.44 (m, 1 H) 6.81 (d, J=2.0 Hz, 1 H) 6.84 (d, J=2.0 Hz, 1 H) 7.21 (d, J=9.5 Hz, 1 H) 7.35 (d, J=9.5 Hz, 1 H) 9.89 - 10.03 (m, 1 H) |
| 313 | 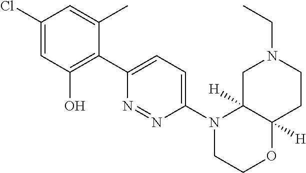 [SMILES: CCN1CC[C@H]2OCCN(c3ccc(-c4c(C)cc(Cl)cc4O)nn3)[C@H]2C1] | 389 0.92 U | 1H NMR (400 MHz, DMSO-d₆, 30° C.) δ ppm 0.97 (t, J = 7.3 Hz, 3 H) 1.76-1.90 (m, 2 H) 2.02-2.13 (m, 1 H) 2.05 (s, 3 H) 2.31-2.41 (m, 3 H) 2.56- 2.65 (m, 2 H) 3.17 (td, J = 12.7, 3.8 Hz, 1 H) 3.62 (td, J = 11.8, 3.3 Hz, 1 H) 3.75 (br q, J = 2.8 Hz, 1 H) 3.94 (br d, J = 12.7 Hz, 1 H) 4.05 (dd, J = 11.8, 3.3 Hz, 1 H) 4.36-4.44 (m, 1 H) 6.81 (d, J = 2.0 Hz, 1 H) 6.84 (d, J = 2.0 Hz, 1 H) 7.21 (d, J = 9.5 Hz, 1 H) 7.35 (d, J = 9.5 Hz, 1 H) 9.88-10.04 (m, 1 H) |
| 314 | 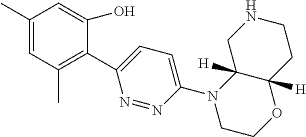 [SMILES: Cc1cc(C)c(-c2ccc(N3CCO[C@H]4CCNC[C@H]43)nn2)c(O)c1] | 341 1.61 G | 1H NMR (400 MHz, DMSO-d₆, 30° C.) δ ppm 1.69- 1.82 (m, 2 H) 2.03 (s, 3 H) 2.22 (s, 3 H) 2.60-2.69 (m, 3 H) 2.92 (t, J = 11.8 Hz, 1 H) 3.14 (td, J = 12.6, 3.8 Hz, 1 H) 3.62 (td, J = 11.8, 3.2 Hz, 1 H) 3.83 (br q, J = 2.6 Hz, 1 H) 3.97 (br d, J = 12.6 Hz, 1 H) 4.06 (dd, J = 11.8, 3.6 Hz, 1 H) 4.17-4.24 (m, 1 H) 6.57 (br s, 1 H) 6.58 (br s, 1 H) 7.19 (d, J = 9.5 Hz, 1 H) 7.33 (d, J = 9.5 Hz, 1 H) 9.38 (br s, 1 H) |
| 315 | 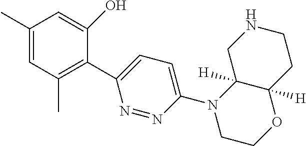 [SMILES: Cc1cc(C)c(-c2ccc(N3CCO[C@@H]4CCNC[C@@H]43)nn2)c(O)c1] | 341 1.61 G | 1H NMR (400 MHz, DMSO-d₆, 30° C.) δ ppm 1.69- 1.82 (m, 2 H) 2.03 (s, 3 H) 2.22 (s, 3 H) 2.60-2.69 (m, 3 H) 2.92 (t, J = 11.8 Hz, 1 H) 3.14 (td, J = 12.6, 3.8 Hz, 1 H) 3.62 (td, J = 11.8, 3.2 Hz, 1 H) 3.83 (br q, J = 2.6 Hz, 1 H) 3.96 (br d, J = 12.6 Hz, 1 H) 4.05 (dd, J = 11.8, 3.6 Hz, 1 H) 4.17-4.25 (m, 1 H) 6.57 (br s, 1 H) 6.58 (br s, 1 H) 7.19 (d, J = 9.5 Hz, 1 H) 7.33 (d, J = 9.5 Hz, 1 H) 9.38 (br s, 1 H) |
| 316 | 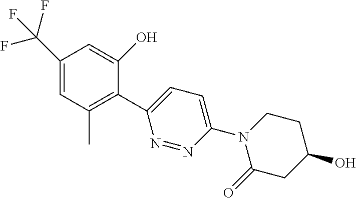 [SMILES: Cc1cc(C(F)(F)F)cc(O)c1-c1ccc(N2CC[C@@H](O)CC2=O)nn1] | 368 2.05 G | 1H NMR (400 MHz, DMSO-d₆, 30° C.) δ ppm 1.86- 1.96 (m, 1 H) 2.07-2.15 (m, 1 H) 2.11 (s, 3 H) 2.43- 2.48 (m partially hidden, 1 H) 2.81 (dd, J = 17.3, 4.8 Hz, 1 H) 3.90-4.00 (m, 1 H) 4.13-4.24 (m, 2 H) 5.15 (br d, J = 3.5 Hz, 1 H) 7.10 (br s, 1 H) 7.16 (br s, 1 H) 7.67 (d, J = 9.0 Hz, 1 H) 8.03 (d, J = 9.0 Hz, 1 H) 10.17-10.42 (m, 1 H) |
| 317 | 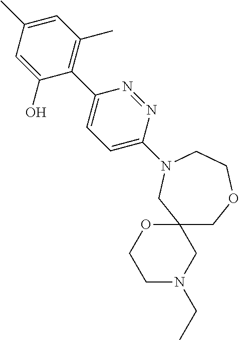 [SMILES: CCN1CCOC2(COCCN(c3ccc(-c4c(C)cc(C)cc4O)nn3)C2)C1] | 399.4 3.02 E | 1H NMR (500 MHz, DMSO-d6) δ (ppm): 9.44 (s, 1H), 7.30 (d, J = 9.5 Hz, 1H), 7.25 (d, J = 9.5 Hz, 1H), 6.65-6.51 (m, 2H), 4.04 (d, J = 15.0 Hz, 1H), 3.96 (s, 2H), 3.85-3.67 (m, 5H), 3.68-3.55 (m, 2H), 3.46-3.39 (m, 2H), 2.41-2.25 (m, 4H), 2.23 (s, 3H), 2.04 (s, 3H), 1.00 (t, J = 7.1 Hz, 3H) |
| 318 | 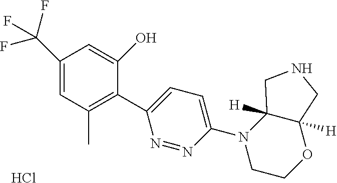 [SMILES: Cc1cc(C(F)(F)F)cc(O)c1-c1ccc(N2CCO[C@@H]3CNC[C@H]32)nn1 ; Cl] | 381.3 2.44 E | 1H NMR (500 MHz, DMSO-d6) δ (ppm): 10.37 (s, 1H), 9.32 (d, J = 27.9 Hz, 1H), 7.63 (s, 2H), 7.15 (s, 1H), 7.13 (s, 1H), 4.45-4.37 (m, 1H), 4.23-4.16 (m, 1H), 3.97 (d, J = 12.4 Hz, 1H), 3.94-3.85 (m, 1H), 3.85-3.76 (m, 1H), 3.36-3.28 (m, 1H), 3.27- 3.19 (m, 1H), 3.12-3.01 (m, 2H), 2.13 (s, 3H). (one proton hidden with water pic, confirmed by HSQC) |
| 320 | 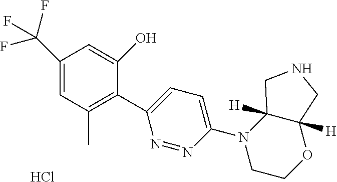 [SMILES: Cc1cc(C(F)(F)F)cc(O)c1-c1ccc(N2CCO[C@H]3CNC[C@H]32)nn1 ; Cl] | 381.4 2.37 E | 1H NMR (400 MHz, MeOD) δ (ppm): 8.22-7.92 (m, 2H), 7.22 (s, 1H), 7.12 (s, 1H), 5.22-5.14 (m, 1H), 4.41-4.36 (m, 1H), 4.24-4.18 (m, 1H), 4.07 (d, J = 13.2 Hz, 1H), 3.90-3.84 (m, 1H), 3.84- 3.77 (m, 1H), 3.63-3.52 (m, 2H), 3.50-3.39 (m, 2H), 2.29 (s, 3H). (OH and NH not visible) |
| 321 | 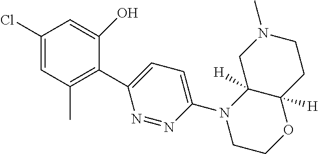 [SMILES: Cc1cc(Cl)cc(O)c1-c1ccc(N2CCO[C@@H]3CCN(C)C[C@@H]32)nn1] | 375 0.89 U | 1H NMR (400 MHz, DMSO-d6, 80° C.) δ ppm 1.74- 1.82 (m, 1 H) 1.83-1.94 (m, 1 H) 1.95-2.05 (m, 1 H) 2.04 (s, 3 H) 2.08-2.17 (m, 1 H) 2.18 (s, 3 H) 2.30-2.40 (m, 1 H) 2.48-2.57 (mpartially hidden, 1 H) 3.12-3.24 (m, 1 H) 3.63 (td, J = 11.6, 3.4 Hz, 1 H) 3.74 (q, J = 2.9 Hz, 1 H) 3.91 (dd, J = 13.1, 2.6 Hz, 1 H) 4.03 (dd, J = 11.6, 3.4 Hz, 1 H) 4.33-4.43 (m, 1 H) 6.47 (br s, 1 H) 6.58 (br s, 1 H) 7.09 (d, J = 9.4 Hz, 1 H) 7.36 (d, J = 9.4 Hz, 1 H) |
| 322 | 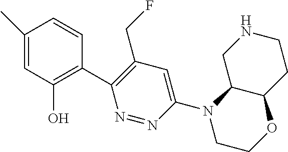 [SMILES: Cc1ccc(-c2nnc(N3CCO[C@@H]4CCNC[C@@H]43)cc2CF)c(O)c1] | 377 0.42 and 1.75 G | 1H NMR (400 MHz, DMSO-d₆, 30° C.) δ ppm 1.74- 1.83 (m, 2 H) 2.29 (s, 3 H) 2.61-2.72 (m, 3 H) 2.97 (t, J = 11.8 Hz, 1 H) 3.20 (td, J = 12.6, 3.8 Hz, 1 H) 3.63 (td, J = 11.8, 3.2 Hz, 1 H) 3.85 (br q, J = 2.6 Hz, 1 H) 4.00-4.13 (m, 2 H) 4.26-4.42 (m, 1 H) 6.77 (t, J = 54.8 Hz, 1 H) 6.73-6.79 (m, 2 H) 7.15 (d, J = 7.5 Hz, 1 H) 7.31 (s, 1 H) 9.58-10.03 (m, 1 H) |
| 323 | 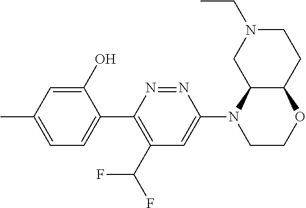 [SMILES: CCN1CC[C@H]2OCCN(c3cc(C(F)F)c(-c4ccc(C)cc4O)nn3)[C@H]2C1] | 405 1.80 G | 1H NMR (400 MHz, DMSO-d6, 30° C.) δ ppm 0.97 (t, J = 7.1 Hz, 3 H), 1.76-1.94 (m, 2 H), 2.10 (td, J = 11.7, 3.2 Hz, 1 H), 2.30 (s, 3 H), 2.32-2.44 (m, 3 H), 2.57-2.66 (m, 2 H), 3.22 (td, J = 12.7, 3.8 Hz, 1 H), 3.63 (td, J = 11.9, 3.1 Hz, 1 H), 3.76 (q, J = 2.8 Hz, 1 H), 3.99 (br d, J = 12.7 Hz, 1 H), 4.05 (dd, J = 11.9, 3.8 Hz, 1 H), 4.51 (br d, J = 10.1 Hz, 1 H), 6.59-6.92 (m, 3 H), 7.16 (d, J = 7.6 Hz, 1 H), 7.30 (s, 1 H), 9.80 (br s, 1 H) |
| 324 | 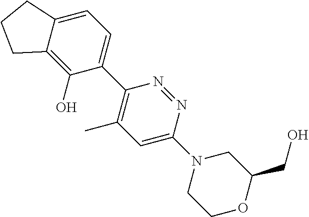 [SMILES: Cc1cc(N2CCO[C@H](CO)C2)nnc1-c1ccc2c(c1O)CCC2] | 342 1.84 G | 1H NMR (400 MHz, DMSO-d6, 30° C.) δ ppm 1.99- 2.10 (m, 2 H), 2.16 (s, 3 H), 2.70 (dd, J = 12.6, 9.8 Hz, 1 H), 2.84 (t, J = 7.5 Hz, 2 H), 2.89 (t, J = 7.6 Hz, 2 H), 2.96 (td, J = 12.3, 3.5 Hz, 1 H), 3.40-3.57 (m, 3 H), 3.60 (td, J = 11.5, 2.6 Hz, 1 H), 3.98 (br dd, J = 11.5, 2.2 Hz, 1 H), 4.13 (br d, J = 12.3 Hz, 1 H), 4.29 (br d, J = 12.6 Hz, 1 H), 4.80 (t, J = 5.5 Hz, 1 H), 6.80 (d, J = 7.6 Hz, 1 H), 7.01 (d, J = 7.6 Hz, 1 H), 7.17 (s, 1 H), 9.40 (s, 1 H) |
| 325 | 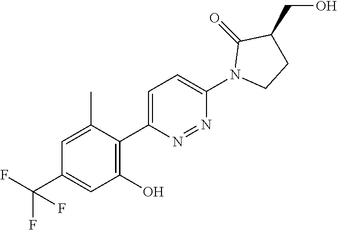 [SMILES: Cc1cc(C(F)(F)F)cc(O)c1-c1ccc(N2CC[C@H](CO)C2=O)nn1] | 368 2.17 G | 1H NMR (400 MHz, DMSO-d₆, 30° C.) δ ppm 2.11 (s, 3 H) 2.12-2.21 (m, 1 H) 2.21-2.32 (m, 1 H) 2.82-2.89 (m, 1 H) 3.64-3.72 (m, 1 H) 3.75-3.82 (m, 1 H) 4.01-4.08 (m, 1 H) 4.15-4.22 (m, 1 H) 4.87 (br t, J = 5.0 Hz, 1 H) 7.09 (br s, 1 H) 7.15 (br s, 1 H) 7.72 (d, J = 9.3 Hz, 1 H) 8.63 (d, J = 9.3 Hz, 1 H) 9.82-10.66 (m, 1 H) |
| 326 | 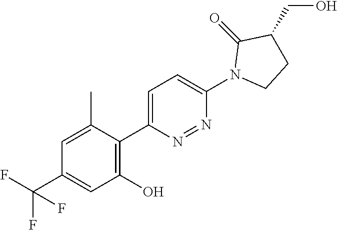 [SMILES: Cc1cc(C(F)(F)F)cc(O)c1-c1ccc(N2CC[C@@H](CO)C2=O)nn1] | 368 2.17 G | 1H NMR (400 MHz, DMSO-d₆, 30° C.) δ ppm 2.11 (s, 3 H) 2.12-2.21 (m, 1 H) 2.21-2.31 (m, 1 H) 2.81-2.90 (m, 1 H) 3.62-3.73 (m, 1 H) 3.74-3.83 (m, 1 H) 4.00-4.08 (m, 1 H) 4.15-4.22 (m, 1 H) 4.87 (br t, J = 5.0 Hz, 1 H) 7.09 (br s, 1 H) 7.15 (s, 1 H) 7.72 (d, J = 9.3 Hz, 1 H) 8.63 (d, J = 9.3 Hz, 1 H) 9.99-10.52 (m, 1 H) |
| 327 | 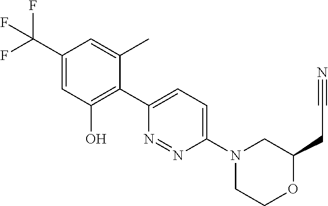 [SMILES: Cc1cc(C(F)(F)F)cc(O)c1-c1ccc(N2CCO[C@H](CC#N)C2)nn1] | 379 2.14 G | 1H NMR (400 MHz, DMSO-d₆, 30° C.) δ ppm 2.12 (s, 3 H) 2.76-2.88 (m, 2 H) 2.91-2.98 (m, 1 H) 3.03 (td, J = 12.6, 2.8 Hz, 1 H) 3.69 (td, J = 11.5, 2.8 Hz, 1 H) 3.76-3.89 (m, 1 H) 4.05 (dd, J = 11.5, 2.8 Hz, 1 H) 4.13 (br d, J = 12.6 Hz, 1 H) 4.43 (br d, J = 12.6 Hz, 1 H) 7.06 (br s, 1 H) 7.12 (br s, 1 H) 7.32 (d, J = 9.5 Hz, 1 H) 7.44 (d, J = 9.5 Hz, 1 H) 9.82-10.58 (m, 1 H) |
| 328 | 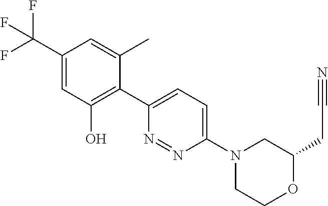 [SMILES: Cc1cc(C(F)(F)F)cc(O)c1-c1ccc(N2CCO[C@@H](CC#N)C2)nn1] | 379 2.13 G | 1H NMR (400 MHz, DMSO-d₆, 30° C.) δ ppm 2.12 (s, 3 H) 2.76-2.88 (m, 2 H) 2.91-2.99 (m, 1 H) 3.03 (td, J = 12.6, 2.8 Hz, 1 H) 3.69 (td, J = 11.5, 2.8 Hz, 1 H) 3.76-3.89 (m, 1 H) 4.05 (dd, J = 11.5, 2.8 Hz, 1 H) 4.13 (br d, J = 12.6 Hz, 1 H) 4.43 (br d, J = 12.6 Hz, 1 H) 7.06 (br s, 1 H) 7.12 (br s, 1 H) 7.33 (d, J = 9.5 Hz, 1 H) 7.45 (d, J = 9.5 Hz, 1 H) 9.95-10.42 (m, 1 H) |
| 329 | 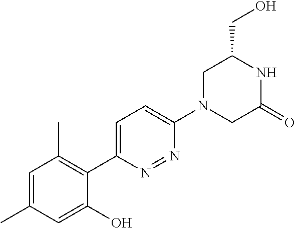 [SMILES: Cc1cc(C)c(-c2ccc(N3CC(=O)N[C@@H](CO)C3)nn2)c(O)c1] | 329 1.7 G | 1H NMR (400 MHz, DMSO-d₆, 30° C.) δ ppm 2.03 (s, 3 H) 2.22 (s, 3 H) 3.35-3.53 (m, 3 H) 3.78 (dd, J = 13.2, 5.5 Hz, 1 H) 3.92 (dd, J = 13.2, 3.5 Hz, 1 H) 4.03-4.18 (m, 2 H) 4.97 (br t, J = 5.3 Hz, 1 H) 6.57 (br s, 1 H) 6.58 (br s, 1 H) 7.26 (d, J = 9.5 Hz, 1 H) 7.35 (d, J = 9.5 Hz, 1 H) 8.05 (br s, 1 H) 9.35 (br s, 1 H) |
| 330 | 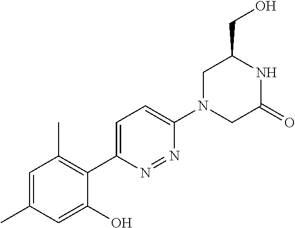 [SMILES: Cc1cc(C)c(-c2ccc(N3CC(=O)N[C@H](CO)C3)nn2)c(O)c1] | 329 1.7 G | 1H NMR (400 MHz, DMSO-d₆, 30° C.) δ ppm 2.03 (s, 3 H) 2.22 (s, 3 H) 3.35-3.52 (m, 3 H) 3.78 (dd, J = 13.2, 5.5 Hz, 1 H) 3.92 (dd, J = 13.2, 3.5 Hz, 1 H) 4.04-4.17 (m, 2 H) 4.98 (br t, J = 5.3 Hz, 1 H) 6.57 (br s, 1 H) 6.58 (br s, 1 H) 7.26 (d, J = 9.5 Hz, 1 H) 7.35 (d, J = 9.5 Hz, 1 H) 8.05 (br s, 1 H) 9.36 (br s, 1 H) |
| 331 | 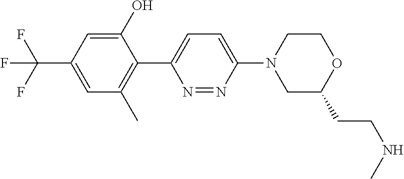 [SMILES: CNCC[C@@H]1CN(c2ccc(-c3c(C)cc(C(F)(F)F)cc3O)nn2)CCO1] | 397 1.75 G | 1H NMR (400 MHz, DMSO-d₆, 30° C.) δ ppm 1.64 (q, J = 6.9 Hz, 2 H) 2.13 (s, 3 H) 2.29 (s, 3 H) 2.61 (br t, J = 6.9 Hz, 2 H) 2.65-2.72 (m, 1 H) 2.98 (td, J = 12.3, 3.4 Hz, 1 H) 3.55-3.63 (m, 2 H) 3.98 (br dd, J = 12.3, 2.4 Hz, 1 H) 4.16 (br d, J = 13.0 Hz, 1 H) 4.26 (br d, J = 13.0 Hz, 1 H) 7.06 (br s, 1 H) 7.11 (br s, 1 H) 7.32 (d, J = 9.5 Hz, 1 H) 7.41 (d, J = 9.5 Hz, 1 H) |
| 332 | 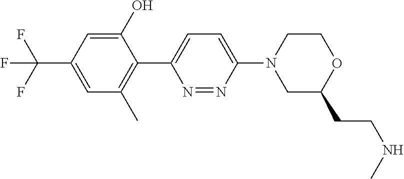 [SMILES: CNCC[C@H]1CN(c2ccc(-c3c(C)cc(C(F)(F)F)cc3O)nn2)CCO1] | 397 1.74 G | 1H NMR (400 MHz, DMSO-d₆, 30° C.) δ ppm 1.64 (q, J = 6.9 Hz, 2 H) 2.13 (s, 3 H) 2.29 (s, 3 H) 2.61 (t, J = 6.9 Hz, 2 H) 2.65-2.73 (m, 1 H) 2.98 (br td, J = 12.3, 3.4 Hz, 1 H) 3.54-3.64 (m, 2 H) 3.98 (br dd, J = 12.3, 2.4 Hz, 1 H) 4.16 (br d, J = 13.0 Hz, 1 H) 4.26 (br d, J = 13.0 Hz, 1 H) 7.06 (br s, 1 H) 7.11 (s, 1 H) 7.32 (d, J = 9.5 Hz, 1 H) 7.41 (d, J = 9.5 Hz, 1 H) |
| 333 | 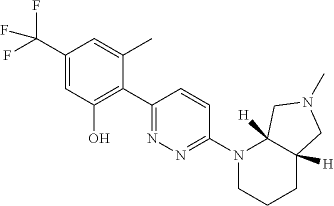 [SMILES: Cc1cc(C(F)(F)F)cc(O)c1-c1ccc(N2CCC[C@H]3CN(C)C[C@H]32)nn1] | 395.4 2.5 E | 1H NMR (500 MHz, DMSO) δ (ppm): 10.16 (s, 1H), 7.42 (d, J = 9.4 Hz, 1H), 7.29 (d, J = 9.5 Hz, 1H), 7.13 (s, 1H), 7.07 (s, 1H), 4.60 (s, 1H), 4.13 (t, J = 4.7 Hz, 1H), 3.99 (dd, J = 11.2, 3.7 Hz, 2H), 3.59 (td, J = 11.8, 2.8 Hz, 1H), 3.23-3.10 (m, 2H), 2.97 (t, J = 7.8 Hz, 1H), 2.58 (m, 2H), 2.38 (s, 3H), 2.13 (s, 3H), 1.24 (s, 1H). |
| 334 | 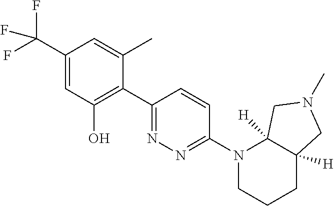 [SMILES: Cc1cc(C(F)(F)F)cc(O)c1-c1ccc(N2CCC[C@@H]3CN(C)C[C@@H]32)nn1] | 395.4 2.5 E | 1H NMR (500 MHz, DMSO) δ (ppm): 10.25- 10.03 (m, 1H), 7.41 (d, J = 9.4 Hz, 1H), 7.28 (d, J = 9.4 Hz, 1H), 7.12 (s, 1H), 7.07 (s, 1H), 4.55 (m, 1H), 4.12 (t, J = 4.8 Hz, 1H), 4.03-3.95 (m, 2H), 3.58 (td, J = 11.8, 2.9 Hz, 1H), 3.20-3.09 (m, 2H), 2.91 (t, J = 7.7 Hz, 1H), 2.58-2.53 (m, 2H), 2.33 (s, 3H), 2.13 (s, 3H). |
| 335 | 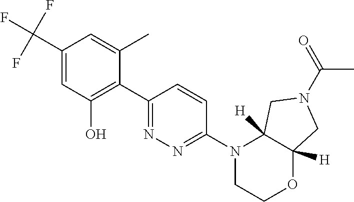 [SMILES: CC(=O)N1C[C@@H]2OCCN(c3ccc(-c4c(C)cc(C(F)(F)F)cc4O)nn3)[C@@H]2C1] | 423.4 2.53 E | 1H NMR (500 MHz, DMSO-d₆) δ (ppm): 9.80 (s, 1H), 7.48-7.39 (m, 1H), 7.38-7.26 (m, 1H), 7.18- 7.07 (m, 2H), 5.06-4.73 (m, 1H), 4.31-4.10 (m, 1H), 4.11-3.95 (m, 2H), 3.91-3.80 (m, 1H), 3.70 (t, J = 11.7 Hz, 1H), 3.59-3.42 (m, 2H), 3.32- 3.15 (m, 2H), 2.17 (s, 3H), 1.96 (s, 3H). |
| 336 | 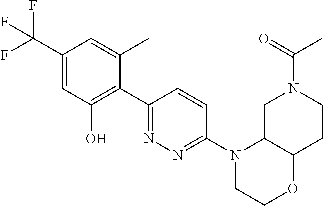 [SMILES: CC(=O)N1CCC2OCCN(c3ccc(-c4c(C)cc(C(F)(F)F)cc4O)nn3)C2C1] | 437.5 2.59 E | ¹H NMR (500 MHz, CD₂Cl₂) δ (ppm): 11.47 (s, 1H), 7.74 (d, J = 9.6 Hz, 1H), 7.51-7.28 (m, 1H), 7.19 (d, J = 5.4 Hz, 1H), 7.14 (d, J = 7.5 Hz, 1H), 5.15-4.92 (m, 1H), 4.18-4.08 (m, 2H), 4.07- 4.01 (m, 1H), 3.98-3.91 (m, 1H), 3.89-3.85 (m, 1H), 3.78-3.69 (m, 1H), 3.54-3.36 (m, 1H), 3.34- 3.19 (m, 1H), 2.77-2.64 (m, 2H), 2.54 (s, 3H), 2.29-2.13 (m, 3H), 2.12-1.96 (m, 1H) |
| 337 | 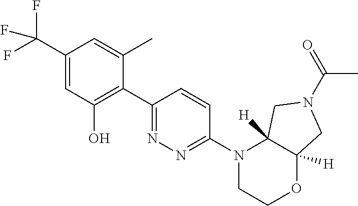 [SMILES: CC(=O)N1C[C@@H]2[C@@H](C1)OCCN2c1ccc(-c2c(C)cc(C(F)(F)F)cc2O)nn1] | 423.4 2.55 E | ¹H NMR (500 MHz, CD2C12) δ (ppm): 11.36 (s, 1H), 7.66 (t, J = 10.0 Hz, 1H), 7.23 (t, J = 9.8 Hz, 1H), 7.16 (s, 1H), 7.10 (d, J = 4.2 Hz, 1H), 4.83- 4.74 (m, 1H), 4.24-4.16 (m, 1H), 4.06-3.97 (m, 1H), 3.96-3.68 (m, 3H), 3.53-3.15 (m, 4H), 2.48 (m, 3H), 2.04 (m, 3H). |
| 338 | 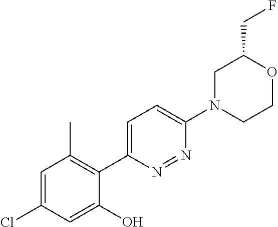 [SMILES: Cc1cc(Cl)cc(O)c1-c1ccc(N2CCO[C@@H](CF)C2)nn1] | 338 2.08 G | 1H NMR (400 MHz, DMSO-d₆, 30° C.) δ ppm 2.05 (s, 3 H) 2.83 (dd, J = 12.6, 10.8 Hz, 1 H) 3.00 (td, J = 12.6, 3.0 Hz, 1 H) 3.66 (td, J = 11.5, 3.0 Hz, 1 H) 3.74-3.87 (m, 1 H) 4.03 (br dd, J = 11.5, 3.0 Hz, 1 H) 4.15 (br d, J = 12.6 Hz, 1 H) 4.33 (br d, J = 12.6 Hz, 1 H) 4.44-4.66 (m, 2 H) 6.81 (d, J = 2.0 Hz, 1 H) 6.84 (d, J = 2.0 Hz, 1 H) 7.31 (d, J = 9.5 Hz, 1 H) 7.39 (d, J = 9.5 Hz, 1 H) 9.84-10.10 (m, 1 H) |
| 339 | 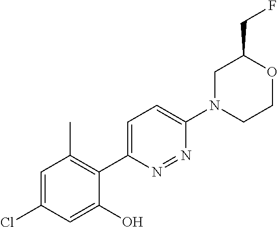 [SMILES: Cc1cc(Cl)cc(O)c1-c1ccc(N2CCO[C@H](CF)C2)nn1] | 338 2.08 G | 1H NMR (400 MHz, DMSO-d₆, 30° C.) δ ppm 2.05 (s, 3 H) 2.83 (dd, J = 12.6, 10.8 Hz, 1 H) 3.00 (td, J = 12.6, 3.0 Hz, 1 H) 3.66 (td, J = 11.5, 3.0 Hz, 1 H) 3.75-3.87 (m, 1 H) 4.03 (br dd, J = 11.5, 3.0 Hz, 1 H) 4.15 (br d, J = 12.6 Hz, 1 H) 4.33 (br d, J = 12.6 Hz, 1 H) 4.45-4.65 (m, 2 H) 6.81 (d, J = 2.0 Hz, 1 H) 6.84 (d, J = 2.0 Hz, 1 H) 7.31 (d, J = 9.5 Hz, 1 H) 7.39 (d, J = 9.5 Hz, 1 H) 9.69-10.16 (m, 1 H) |
| 340 | 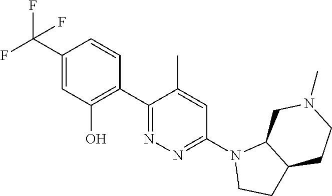 [SMILES: Cc1cc(N2CC[C@@H]3CCN(C)C[C@@H]32)nnc1-c1ccc(C(F)(F)F)cc1O] | 393 0.47 and 1.80 G | 1H NMR (400 MHz, DMSO-d₆, 30° C.) ppm 1.66- 1.78 (m, 2 H) 1.85-2.05 (m, 3 H) 2.08 (s, 3 H) 2.09- 2.14 (m, 1 H) 2.17 (s, 3 H) 2.31-2.40 (m, 1 H) 2.52-2.58 (m, 1 H) 3.04-3.10 (m, 1 H) 3.34-3.43 (m, 1 H) 3.50-3.59 (m, 1 H) 4.16-4.26 (m, 1 H) 6.77 (s, 1 H) 7.20 (br s, 1 H) 7.22 (br d, J = 7.8 Hz, 1 H) 7.40 (d, J = 7.8 Hz, 1 H) 10.32 - 10.54 (m, 1 H) |
| 341 | 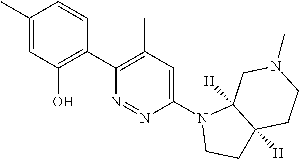 [SMILES: Cc1ccc(-c2nnc(N3CC[C@@H]4CCN(C)C[C@@H]43)cc2C)c(O)c1] | 339 1.44 G | 1H NMR (400 MHz, DMSO-d₆, 30° C.) δ ppm 1.66- 1.77 (m, 2 H) 1.85-2.07 (m, 3 H) 2.07-2.13 (m, 1 H) 2.09 (s, 3 H) 2.17 (s, 3 H) 2.28 (s, 3 H) 2.30- 2.39 (m, 1 H) 2.52-2.56 (m, 1 H) 3.02-3.09 (m, 1 H) 3.34-3.42 (m, 1 H) 3.49-3.57 (m, 1 H) 4.15- 4.24 (m, 1 H) 6.69 (br d, J = 7.8 Hz, 1 H) 6.73 (br s, 2 H) 7.05 (d, J = 7.8 Hz, 1 H) 9.69 (s, 1 H) |
| 342 | 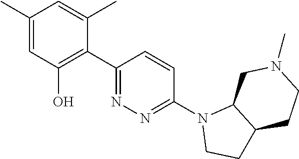 [SMILES: Cc1cc(C)c(-c2ccc(N3CC[C@@H]4CCN(C)C[C@@H]43)nn2)c(O)c1] | 339 0.46 and 1.58 G | 1H NMR (400 MHz, DMSO-d6, 30° C.) d ppm 1.66- 1.81 (m, 2 H), 1.82-2.06 (m, 3 H), 2.04 (s, 3 H), 2.06-2.15 (m, 1 H), 2.17 (s, 3 H), 2.22 (s, 3 H), 2.31-2.40 (m, 1 H), 2.52-2.58 (m partially hidden, 1 H), 3.06 (br dd, J = 6.0 and 10.5 Hz, 1 H), 3.35-3.48 (m, 1 H), 3.50-3.60 (m, 1 H), 4.15- 4.25 (m, 1 H), 6.56 (br s, 1 H), 6.57 (br s, 1 H), 6.87 (d, J = 9.5 Hz, 1 H), 7.25 (d, J = 9.5 Hz, 1 H), 9.39 (s, 1 H) |
| 343 | 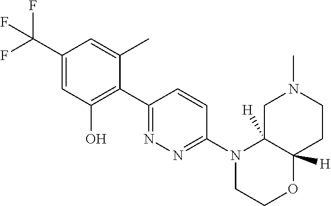 [SMILES: Cc1cc(C(F)(F)F)cc(O)c1-c1ccc(N2CCO[C@H]3CCN(C)C[C@@H]32)nn1] | 409.4 2.73 E | ¹H NMR (500 MHz, DMSO) δ (ppm) : 10.21 (br. s, 1H), 7.51 (d, J = 9.2 Hz, 1H), 7.38 (d, J = 9.2 Hz, 1H), 7.14 (s, 1H), 7.08 (s, 1H), 3.97-3.91 (m, 1H), 3.88-3.83 (m, 1H), 3.72-3.64 (m, 1H), 3.56- 3.39 (m, 4H), 2.84 (d, J = 11.3 Hz, 1H), 2.19 (s, 3H), 2.12 (s, 4H), 1.94-1.73 (m, 2H), 1.73-1.54 (m, 1H). |
| 344 | 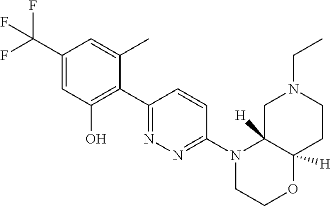 [SMILES: CCN1CC[C@H]2OCCN(c3ccc(-c4c(C)cc(C(F)(F)F)cc4O)nn3)[C@@H]2C1] | 423.5 2.70 E | ¹H NMR (500 MHz, DMSO-d₆) δ (ppm): 10.22 (s, 1H), 7.52 (d, J = 9.2 Hz, 1H), 7.38 (d, J = 9.2 Hz, 1H), 7.15 (s, 1H), 7.09 (s, 1H), 3.98-3.91 (m, 1H), 3.90-3.84 (m, 1H), 3.74-3.66 (m, 1H), 3.59- 3.50 (m, 2H), 3.49-3.38 (m, 2H), 2.94 (d, J = 11.3 Hz, 1H), 2.40-2.33 (m, 2H), 2.13 (s, 3H), 2.11- 2.02 (m, 1H), 1.89-1.83 (m, 1H), 1.76 (t, J = 10.1 Hz, 1H), 1.67-1.57 (m, 1H), 0.97 (t, J = 7.1 Hz, 3H). |
| 345 | 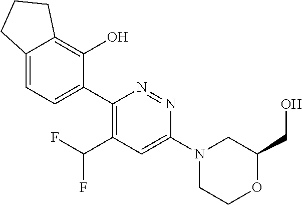 [SMILES: OC[C@@H]1CN(c2cc(C(F)F)c(-c3ccc4c(c3O)CCC4)nn2)CCO1] | 378 2.17 G | 1H NMR (400 MHz, DMSO-d6, 30°° C.) 8 ppm 2.05 (quin, J = 7.4 Hz, 2 H), 2.79 (dd, J = 12.8, 9.9 Hz, 1 H), 2.83-2.94 (m, 4 H), 3.04 (td, J = 12.8, 3.5 Hz, 1 H), 3.43-3.58 (m, 3 H), 3.62 (td, J = 11.6, 2.8 Hz, 1 H), 4.00 (br dd, J = 11.6, 2.2 Hz, 1 H), 4.24 (br d, J = 12.9 Hz, 1 H), 4.38 (br d, J = 12.8 Hz, 1 H), 4.81 (br t, J = 5.5 Hz, 1 H), 6.76 (t, J = 54.5 Hz, 1 H), 6.84 (d, J = 7.6 Hz, 1 H), 7.03 (d, J = 7.5 Hz, 1 H), 7.40 (s, 1 H), 9.04-9.19 (m, 1 H) |
| 346 | 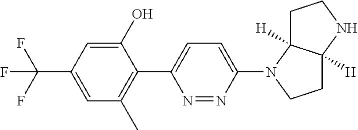 [SMILES: Cc1cc(C(F)(F)F)cc(O)c1-c1ccc(N2CC[C@@H]3NCC[C@@H]32)nn1] | 365 0.47 and 1.81 G | 1H NMR (400 MHz, DMSO-d₆, 100° C.) δ ppm 2.12- 2.22 (m, 1 H) 2.20 (s, 3 H) 2.33-2.48 (m, 3 H) 3.23-3.37 (m, 2 H) 3.77-3.90 (m, 2 H) 4.43-4.51 (m, 1 H) 4.80-4.87 (m, 1 H) 7.12 (br s, 1 H) 7.16 (br s, 1 H) 7.29 (d, J = 9.5 Hz, 1 H) 7.58 (d, J = 9.5 Hz, 1 H) 9.40-9.89 (m, 2 H) |
| 348 | 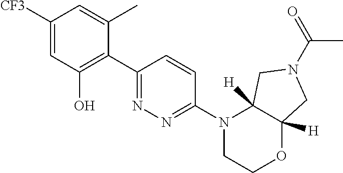 [SMILES: CC(=O)N1C[C@@H]2OCCN(c3ccc(-c4c(C)cc(C(F)(F)F)cc4O)nn3)[C@@H]2C1] | 423.3 2.68 E | ¹H NMR (500 MHz, DMSO-d₆) δ (ppm): 10.16 (s, 1H), 7.53-7.42 (m, 1H), 7.42-7.29 (m, 1H), 7.13 (s, 1H), 7.07 (s, 1H), 5.03-4.80 (m, 1H), 4.34- 4.11 (m, 1H), 4.10-3.94 (m, 2H), 3.90 - 3.43 (m, 5H), 3.23 - 3.08 (m, 1H), 2.13 (d, J = 3.1 Hz, 3H), 1.95 (d, J = 4.9 Hz, 3H). |
| 349 | 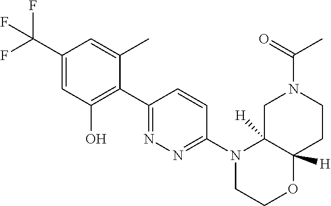 [SMILES: CC(=O)N1CC[C@@H]2OCCN(c3ccc(-c4c(C)cc(C(F)(F)F)cc4O)nn3)[C@H]2C1] | 437.5 2.57 E | ¹H NMR (500 MHz, DMSO-d₆) δ (ppm): 10.23 (s, 1H), 7.65-7.38 (m, 2H), 7.14 (s, 1H), 7.09 (s, 1H), 4.71 (d, J = 13.2 Hz, 1H), 4.62-4.44 (m, 1H), 4.07- 3.84 (m, 2H), 3.83-3.63 (m, 3H), 3.29-3.11 (m, 1H), 2.80-2.66 (m, 1H), 2.21-1.99 (m, 6H), 1.98- 1.83 (m, 1H), 1.75-1.64 (m, 1H), 1.32-1.25 (m, 1H). |
| 350 | 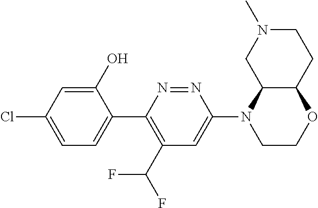 [SMILES: CN1CC[C@H]2OCCN(c3cc(C(F)F)c(-c4ccc(Cl)cc4O)nn3)[C@H]2C1] | 411 0.95 U | 1H NMR (400 MHz, DMSO-d6, 30° C.) d ppm 1.76- 1.95 (m, 2 H), 2.06-2.14 (m, 1 H), 2.18 (s, 3 H), 2.30-2.40 (m, 1 H), 2.51-2.57 (m hidden, 2 H), 3.17-3.26 (m partially hidden, 1 H), 3.63 (td, J = 3.2 and 11.8 Hz, 1 H), 3.74 (br q, J = 2.8 Hz, 1 H), 3.96- 4.10 (m, 2 H), 4.47-4.59 (m, 1 H), 6.80 (t, J = 54.3 Hz, 1 H), 6.96-7.01 (m, 2 H), 7.29 (br d, J = 8.1 Hz, 1 H), 7.33 (br s, 1 H), 10.18-10.70 (m, 1 H) |
| 351 | 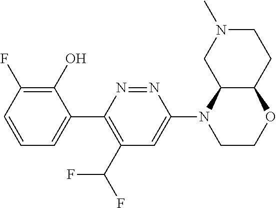 [SMILES: CN1CC[C@H]2OCCN(c3cc(C(F)F)c(-c4cccc(F)c4O)nn3)[C@H]2C1] | 395 0.85 U | 1H NMR (400 MHz, DMSO-d6, 30° C.) d ppm 1.76- 1.95 (m, 2 H), 2.06-2.15 (m, 1 H), 2.18 (s, 3 H), 2.30-2.40 (m, 1 H), 2.51-2.57 (m hidden, 2 H), 3.12-3.26 (m partially hidden, 1 H), 3.63 (td, J = 3.2 and 11.8 Hz, 1 H), 3.75 (q, J = 2.8 Hz, 1 H), 3.98- 4.11 (m, 2 H), 4.48-4.57 (m, 1 H), 6.79 (br t, J = 54.3 Hz, 1 H), 6.90-6.96 (m, 1 H), 7.09 (br d, J = 7.4 Hz, 1 H), 7.25-7.32 (m, 1 H), 7.35 (br s, 1 H), 9.71-10.31 (m, 1 H) |
| 352 | 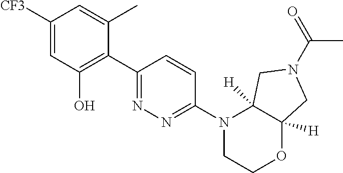 [SMILES: CC(=O)N1C[C@H]2OCCN(c3ccc(-c4c(C)cc(C(F)(F)F)cc4O)nn3)[C@H]2C1] | 423.3 2.68 E | ¹H NMR (500 MHz, DMSO-d₆) δ (ppm): 10.19 (s, 1H), 7.55-7.41 (m, 1H), 7.42-7.28 (m, 1H), 7.12 (s, 1H), 7.06 (s, 1H), 4.97-4.79 (m, 1H), 4.28- 4.11 (m, 1H), 4.10-3.94 (m, 2H), 3.84-3.73 (m, 1H), 3.72-3.60 (m, 1H), 3.58-3.48 (m, 1H), 3.48- 3.42 (m, 1H), 3.25-3.08 (m, 2H), 2.13 (d, J = 3.0 Hz, 3H), 1.95 (d, J = 4.8 Hz, 3H). Mixture of rotamers |
| 353 | 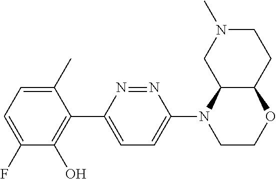 [SMILES: Cc1ccc(F)c(O)c1-c1ccc(N2CCO[C@@H]3CCN(C)C[C@@H]32)nn1] | 359 0.98 Z1 | 1H NMR (400 MHz, DMSO-d₆, 30° C.) δ ppm 1.76- 1.95 (m, 2 H) 2.04 (s, 3 H) 2.13-2.21 (m, 1 H) 2.25 (br s, 3 H) 2.37-2.47 (m, 1 H) 2.54-2.64 (m, 2 H) 3.17 (td, J = 12.7, 3.8 Hz, 1 H) 3.64 (td, J = 11.8, 3.3 Hz, 1 H) 3.73-3.79 (m, 1 H) 3.94 (br d, J = 12.7 Hz, 1 H) 4.06 (dd, J = 11.8, 3.3 Hz, 1 H) 4.40-4.52 (m, 1 H) 6.75 (dd, J = 8.4, 5.0 Hz, 1 H) 7.11 (dd, J = 10.8, 8.4 Hz, 1 H) 7.25 (d, J = 9.5 Hz, 1 H) 7.40 (d, J = 9.5 Hz, 1 H) 9.58 (br s, 1 H) |
| 354 | 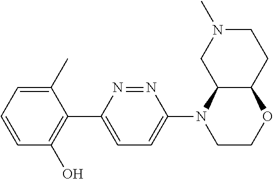 [SMILES: Cc1cccc(O)c1-c1ccc(N2CCO[C@@H]3CCN(C)C[C@@H]32)nn1] | 341 0.69 and 0.72 U | 1H NMR (400 MHz, DMSO-d₆, 30° C.) δ ppm 1.76- 1.93 (m, 2 H) 2.06 (s, 3 H) 2.07-2.13 (m, 1 H) 2.18 (s, 3 H) 2.30-2.36 (m, 1 H) 2.51-2.56 (m partially hidden, 2 H) 3.16 (td, J = 12.7, 3.8 Hz, 1 H) 3.63 (td, J = 11.8, 3.3 Hz, 1 H) 3.72-3.77 (m, 1 H) 3.93 (br d, J = 12.7 Hz, 1 H) 4.05 (dd, J = 11.8, 3.3 Hz, 1 H) 4.36-4.45 (m, 1 H) 6.73-6.79 (m, 2 H) 7.10 (t, J = 7.6 Hz, 1 H) 7.21 (d, J = 9.5 Hz, 1 H) 7.36 (d, J = 9.5 Hz, 1 H) 9.42 (s, 1 H) |
| 355 | 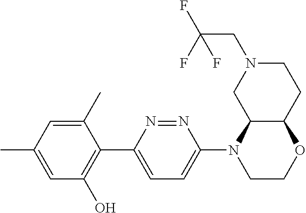 [SMILES: Cc1cc(C)c(-c2ccc(N3CCO[C@@H]4CCN(CC(F)(F)F)C[C@@H]43)nn2)c(O)c1] | 423 1.32 U | 1H NMR (400 MHz, DMSO-d₆, 30° C.) δ ppm 1.76- 1.95 (m, 2 H) 2.03 (s, 3 H) 2.22 (s, 3 H) 2.52 - 2.61 (m, 1 H) 2.64-2.74 (m, 2 H) 2.76-2.88 (m, 1 H) 3.11-3.27 (m, 3 H) 3.64 (td, J = 11.8, 3.3 Hz, 1 H) 3.73-3.79 (m, 1 H) 3.92 (br d, J = 12.7 Hz, 1 H) 4.06 (dd, J = 11.8, 3.3 Hz, 1 H) 4.40-4.48 (m, 1 H) 6.57 (br s, 1 H) 6.58 (br s, 1 H) 7.20 (d, J = 9.5 Hz, 1 H) 7.35 (d, J = 9.5 Hz, 1 H) 9.37 (br s, 1 H) |
| 356 | 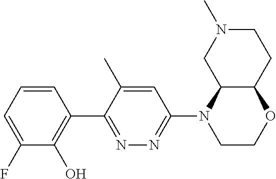 [SMILES: Cc1cc(N2CCO[C@@H]3CCN(C)C[C@@H]32)nnc1-c1cccc(F)c1O] | 359 0.74 U | 1H NMR (400 MHz, DMSO-d₆, 30° C.) δ ppm 1.75- 1.92 (m, 2 H) 2.06-2.13 (m, 1 H) 2.10 (s, 3 H) 2.17 (s, 3 H) 2.26-2.36 (m, 1 H) 2.51-2.54 (m, partially hidden, 2 H) 3.15 (td, J = 12.7, 3.8 Hz, 1 H) 3.61 (td, J = 11.8, 3.3 Hz, 1 H) 3.68-3.76 (m, 1 H) 3.93 (br d, J = 12.7 Hz, 1 H) 4.04 (dd, J = 11.8, 3.3 Hz, 1 H) 4.37-4.45 (m, 1 H) 6.90 (td, J = 7.8, 5.0 Hz, 1 H) 7.03 (dt, J = 7.8, 1.3 Hz, 1 H) 7.12 (s, 1 H) 7.20-7.27 (m, 1 H) 9.79-10.00 (m, 1 H) |
| 357 | 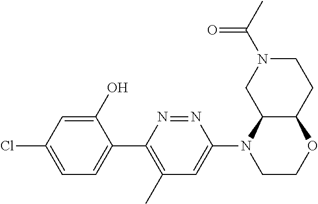 [SMILES: CC(=O)N1CC[C@H]2OCCN(c3cc(C)c(-c4ccc(Cl)cc4O)nn3)[C@H]2C1] | 403 1.11 U | 1H NMR (400 MHz, DMSO-d₆, 100° C.) δ ppm 1.77- 1.91 (m, 2 H) 2.02 (s, 3 H) 2.13 (br s, 3 H) 3.19- 3.27 (m, 1 H) 3.52-4.45 (m, 4 H) 3.68 (td, J = 11.8, 3.3 Hz, 1 H) 3.81-3.91 (m, 1 H) 3.96 (br d, J = 12.7 Hz, 1 H) 4.05-4.11 (m, 1 H) 4.24-4.39 (m, 1 H) 6.92 (dd, J = 8.1, 2.1 Hz, 1 H) 6.97 (d, J = 2.1 Hz, 1 H) 7.07 (s, 1 H) 7.22 (d, J = 8.1 Hz, 1 H) 8.83- 10.92 (m, 1 H) |
| 358 | 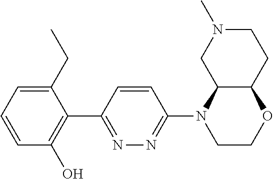 [SMILES: CCc1cccc(O)c1-c1ccc(N2CCO[C@@H]3CCN(C)C[C@@H]32)nn1] | 355 0.80 U | 1H NMR (400 MHz, DMSO-d₆, 30° C.) δ ppm 0.95 (t, J = 7.6 Hz, 3 H) 1.76-1.93 (m, 2 H) 2.06-2.14 (m, 1 H) 2.18 (s, 3 H) 2.30-2.42 (m, 3 H) 2.51- 2.56 (m partially hidden, 2 H) 3.16 (td, J = 12.7, 3.8 Hz, 1 H) 3.63 (td, J = 11.8, 3.3 Hz, 1 H) 3.72-3.77 (m, 1 H) 3.93 (br d, J = 12.7 Hz, 1 H) 4.05 (dd, J = 11.8, 3.3 Hz, 1 H) 4.36-4.45 (m, 1 H) 6.77 (d, J = 7.8 Hz, 2 H) 7.14 (t, J = 7.8 Hz, 1 H) 7.20 (d, J = 9.5 Hz, 1 H) 7.33 (d, J = 9.5 Hz, 1 H) 9.32 (s, 1 H) |
| 359 | 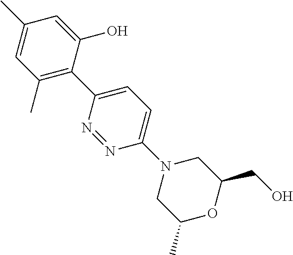 [SMILES: Cc1cc(C)c(-c2ccc(N3C[C@@H](CO)O[C@H](C)C3)nn2)c(O)c1] | 330 0.91 B | 1H NMR (400 MHz, DMSO-d₆, 30° C.) δ ppm 1.19 (d, J = 6.5 Hz, 3 H) 2.03 (s, 3 H) 2.22 (s, 3 H) 3.21 (dd, J = 12.7, 7.0 Hz, 1 H) 3.49-3.58 (m, 3 H) 3.62- 3.68 (m, 1 H) 3.81 (dd, J = 12.7, 3.3 Hz, 1 H) 3.84- 3.90 (m, 1 H) 3.99-4.08 (m, 1 H) 4.72 (t, J = 5.7 Hz, 1 H) 6.57 (br s, 1 H) 6.58 (br s, 1 H) 7.23 (d, J = 9.5 Hz, 1 H) 7.33 (d, J = 9.5 Hz, 1 H) 9.36 (s, 1 H) |
| 360 | 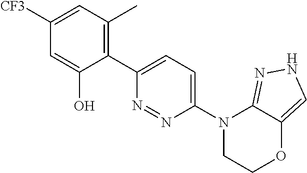 [SMILES: Cc1cc(C(F)(F)F)cc(O)c1-c1ccc(N2CCOc3c[nH]nc32)nn1] | 378.4 2.77 E | 1H NMR (400 MHz, DMSO) δ (ppm): 11.99 (s, 1H), 10.22 (s, 1H), 8.63 (d, J = 9.4 Hz, 1H), 7.59 (d, J = 9.4 Hz, 1H), 7.48 (d, J = 1.7 Hz, 1H), 7.15 (s, 1H), 7.09 (s, 1H), 4.38-4.20 (m, 4H), 2.14 (s, 3H). |
| 361 | 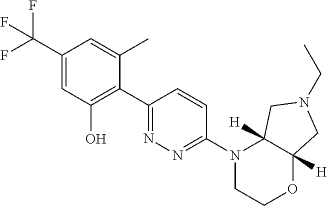 [SMILES: CCN1C[C@@H]2OCCN(c3ccc(-c4c(C)cc(C(F)(F)F)cc4O)nn3)[C@@H]2C1] | 409.5 2.51 E | ¹H NMR (500 MHz, DMSO-d₆) δ (ppm): 10.16 (s, 1H), 7.41 (d, J = 9.4 Hz, 1H), 7.29 (d, J = 9.5 Hz, 1H), 7.12 (s, 1H), 7.07 (d, J = 1.8 Hz, 1H), 4.68- 4.46 (m, 1H), 4.10 (t, J = 4.7 Hz, 1H), 4.03-3.96 (m, 2H), 3.58 (td, J = 11.8, 2.9 Hz, 1H), 3.19-3.10 (m, 2H), 2.96 (t, J = 7.7 Hz, 1H), 2.57-2.52 (m, 4H), 2.13 (s, 3H), 1.01 (t, J = 7.2 Hz, 3H). |
| 362 | 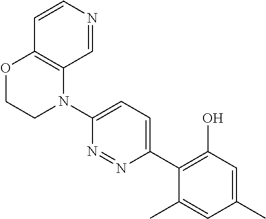 [SMILES: Cc1cc(C)c(-c2ccc(N3CCOc4ccncc43)nn2)c(O)c1] | 335 0.82 and 0.83 B | 1H NMR (400 MHz, DMSO-d₆, 30° C.) δ ppm 2.07 (br s, 3 H) 2.24 (s, 3 H) 4.14-4.18 (m, 2 H) 4.38- 4.43 (m, 2 H) 6.60 (br s, 1 H) 6.61 (br s, 1 H) 6.97 (d, J = 5.4 Hz, 1 H) 7.50 (d, J = 9.3 Hz, 1 H) 7.61 (d, J = 9.3 Hz, 1 H) 8.07 (d, J = 5.4 Hz, 1 H) 8.66 (s, 1 H) 9.40 (br s, 1 H) |
| 363 | 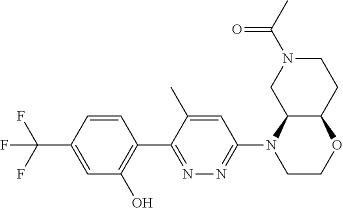 [SMILES: CC(=O)N1CC[C@H]2OCCN(c3cc(C)c(-c4ccc(C(F)(F)F)cc4O)nn3)[C@H]2C1] | 437 1.21 U | 1H NMR (400 MHz, DMSO-d6, 100° C.) d ppm 1.78-1.94 (m, 2 H), 2.04 (s, 3 H), 2.15 (br s, 3 H), 2.71-3.21 (m partially hidden, 2 H), 3.26 (td, J = 3.8 and 12.6 Hz, 1 H), 3.50-4.24 (m partially hidden, 2 H), 3.71 (td, J = 3.2 and 11.8 Hz, 1 H), 3.89 (br q, J = 2.8 Hz, 1 H), 4.00 (br d, J = 12.6 Hz, 1 H), 4.11 (dd, J = 3.8 and 11.8 Hz, 1 H), 4.27-4.44 (m, 1 H), 7.11 (br s, 1 H), 7.21-7.25 (m, 2 H), 7.45 (d, J = 7.8 Hz, 1 H) |
| 364 | 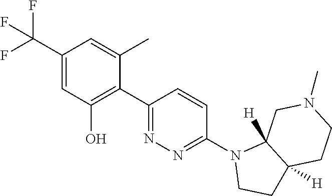 [SMILES: Cc1cc(C(F)(F)F)cc(O)c1-c1ccc(N2CC[C@@H]3CCN(C)C[C@H]32)nn1] | 393.4 2.69 E | ¹H NMR (400 MHz, DMSO-d6) δ (ppm): 10.17 (s, 1H), 7.32 (d, J = 9.2 Hz, 1H), 7.24-7.08 (m, 2H), 7.06 (s, 1H), 4.18-3.96 (m, 1H), 3.90-3.71 (m, 1H), 3.66 (d, J = 13.9 Hz, 1H), 3.24-3.11 (m, 1H), 3.08-2.85 (m, 1H), 2.41-2.31 (m, 2H), 2.29- 2.22 (m, 3H), 2.18-2.08 (m, 4H), 1.98-1.79 (m, 2H), 1.70-1.56 (m, 1H), 1.53-1.38 (m, 1H). |
| 365 | 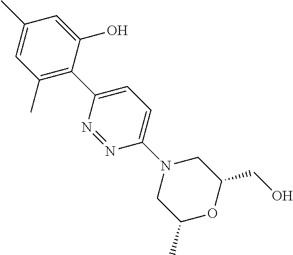 [SMILES: Cc1cc(C)c(-c2ccc(N3C[C@H](CO)O[C@H](C)C3)nn2)c(O)c1] | 330 0.93 B | 1H NMR (400 MHz, DMSO-d₆, 30° C.) δ ppm 1.20 (d, J = 6.0 Hz, 3 H) 2.03 (s, 3 H) 2.22 (s, 3 H) 2.52- 2.65 (m, 2 H) 3.43-3.50 (m, 1 H) 3.50-3.63 (m, 2 H) 3.63-3.72 (m, 1 H) 4.24 (br d, J = 12.5 Hz, 1 H) 4.35 (br d, J = 12.5 Hz, 1 H) 4.79 (t, J = 5.5 Hz, 1 H) 6.57 (br s, 1 H) 6.58 (br s, 1 H) 7.26 (d, J = 9.5 Hz, 1 H) 7.34 (d, J = 9.5 Hz, 1 H) 9.34-9.40 (m, 1 H) |
| 366 | 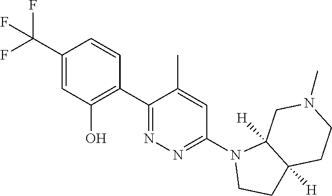 [SMILES: Cc1cc(N2CC[C@@H]3CCN(C)C[C@@H]32)nnc1-c1ccc(C(F)(F)F)cc1O] | 393 0.87 U | 1H NMR (400 MHz, DMSO-d6, 30° C.) d ppm 1.65- 1.78 (m, 2 H), 1.86-2.05 (m, 3 H), 2.06-2.13 (m, 1 H), 2.08 (s, 3 H), 2.17 (s, 3 H), 2.31-2.40 (m, 1 H), 2.52-2.57 (m partially hidden, 1 H), 3.07 (br dd, J = 6.0 and 10.5 Hz, 1 H), 3.36-3.42 (m, 1 H), 3.51-3.59 (m, 1 H), 4.16-4.27 (m, 1 H), 6.77 (s, 1 H), 7.20 (br s, 1 H), 7.22 (br d, J = 7.8 Hz, 1 H), 7.40 (d, J = 7.8 Hz, 1 H), 10.22-10.72 (m, 1 H) |
| 367 | 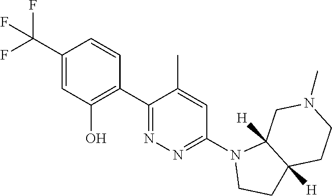 [SMILES: Cc1cc(N2CC[C@H]3CCN(C)C[C@H]32)nnc1-c1ccc(C(F)(F)F)cc1O] | 393 0.87 U | 1H NMR (400 MHz, DMSO-d6, 30° C.) d ppm 1.65- 1.79 (m, 2 H), 1.88-2.06 (m, 3 H), 2.06-2.14 (m, 1 H), 2.08 (s, 3 H), 2.17 (s, 3 H), 2.29-2.38 (m, 1 H), 2.54-2.56 (m partially hidden, 1 H), 3.07 (br dd, J = 6.0 and 10.5 Hz, 1 H), 3.38- 3.43 (m, 1 H), 3.51-3.59 (m, 1 H), 4.14-4.28 (m, 1 H), 6.77 (s, 1 H), 7.20 (br s, 1 H), 7.22 (br d, J = 7.8 Hz, 1 H), 7.40 (d, J = 7.8 Hz, 1 H), 10.23-10.67 (m, 1 H) |
| 368 | 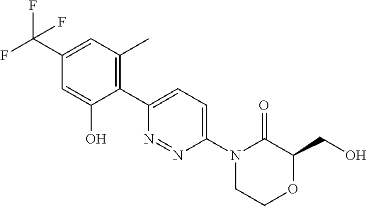 [SMILES: Cc1cc(C(F)(F)F)cc(O)c1-c1ccc(N2CCO[C@H](CO)C2=O)nn1] | 384 1.22 B | 1H NMR (400 MHz, DMSO-d₆, 30° C.) δ ppm 2.12 (s, 3 H) 3.76 (br d, J = 12.5 Hz, 1 H) 3.90 (br d, J = 12.5 Hz, 1 H) 3.94-4.04 (m, 1 H) 4.07-4.15 (m, 1 H) 4.19-4.30 (m, 2 H) 4.37 (dd, J = 4.0, 2.5 Hz, 1 H) 4.91-5.05 (m, 1 H) 7.10 (br s, 1 H) 7.15 (br s, 1 H) 7.74 (d, J = 9.3 Hz, 1 H) 8.32 (d, J = 9.3 Hz, 1 H) 9.97-10.74 (m, 1 H) |
| 369 | 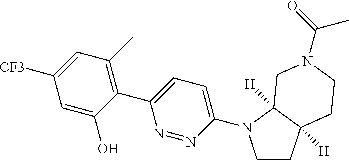 [SMILES: CC(=O)N1CC[C@H]2CCN(c3ccc(-c4c(C)cc(C(F)(F)F)cc4O)nn3)[C@H]2C1] | 421.5 2.74 E | ¹H NMR (500 MHz, DMSO-d₆) δ (ppm): 9.40 (s, 1H), 7.37 (d, J = 9.2 Hz, 1H), 7.12 (s, 1H), 7.06 (s, 1H), 7.04-6.80 (m, 1H), 4.62 (dd, J = 12.9, 5.9 Hz, 1H), 4.34-4.06 (m, 1H), 3.79-3.54 (m, 2H), 3.51-3.36 (m, 2H), 3.23-3.07 (m, 1H), 2.69- 2.60 (m, 1H), 2.27-2.04 (m, 4H), 2.06-1.89 (m, 4H), 1.85-1.71 (m, 1H), 1.68-1.55 (m, 1H), mixture of rotamers. |
| 370 | 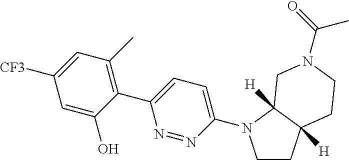 [SMILES: CC(=O)N1CC[C@@H]2CCN(c3ccc(-c4c(C)cc(C(F)(F)F)cc4O)nn3)[C@@H]2C1] | 421.5 2.74 E | ¹H NMR (400 MHz, DMSO- d₆) δ (ppm): 9.40 (s, 1H), 7.38 (dd, J = 9.3, 1.0 Hz, 1H), 7.13 (s, 1H), 7.06 (s, 1H), 7.03-6.82 (m, 1H), 4.63 (dd, J = 12.8, 5.9 Hz, 1H), 4.30-4.10 (m, 1H), 3.81-3.54 (m, 2H), 3.53-3.39 (m, 2H), 3.29-3.11 (m, 1H), 2.62 (dd, J = 12.9, 10.0 Hz, 1H), 2.24-2.08 (m, 4H), 2.07-1.88 (m, 4H), 1.87-1.71 (m, 1H), 1.68- 1.39 (m, 1H), , mixture of rotamers. |
| 371 | 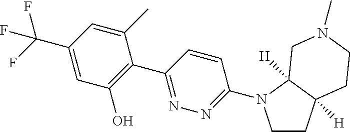 [SMILES: Cc1cc(C(F)(F)F)cc(O)c1-c1ccc(N2CC[C@@H]3CCN(C)C[C@@H]32)nn1] | 393.5 2.66 E | ¹H NMR (400 MHz, DMSO-d₆) δ (ppm): 10.14 (s, 1H), 7.31 (d, J = 9.3 Hz, 1H), 7.12 (d, J = 1.8 Hz, 1H), 7.06 (d, J = 1.8 Hz, 1H), 6.93 (d, J = 9.3 Hz, 1H), 4.33-4.15 (m, 1H), 3.67-3.53 (m, 1H), 3.48- 3.37 (m, 1H), 3.12-3.03 (m, 1H), 2.61-2.53 (m, 1H), 2.43-2.35 (m, 1H), 2.18 (s, 3H), 2.14 (s, 3H), 2.12-1.87 (m, 4H), 1.83-1.66 (m, 2H). |
| 372 | 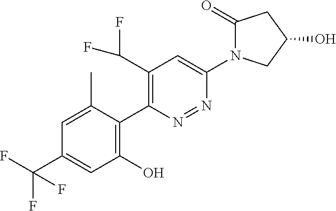 [SMILES: Cc1cc(C(F)(F)F)cc(O)c1-c1nnc(N2C[C@@H](O)CC2=O)cc1C(F)F] | 404 1.33 and 1.36 B | 1H NMR (400 MHz, DMSO-d₆, 100° C.) δ ppm 2.01 (br s, 3 H) 2.49-2.52 (m hidden, 1 H) 2.96-3.02 (m partially hidden, 1 H) 4.11-4.19 (m, 1 H) 4.23- 4.33 (m, 1 H) 4.48-4.55 (m, 1 H) 5.12-5.16 (m, 1 H) 6.69 (t, J = 54.0 Hz, 1 H) 7.07 (br s, 1 H) 7.11 (br s, 1 H) 8.84 (br s, 1 H) 9.23-10.80 (m, 1 H) |
| 373 | 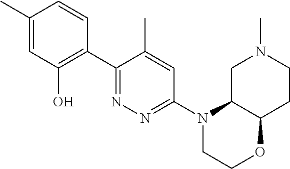 [SMILES: Cc1ccc(-c2nnc(N3CCO[C@@H]4CCN(C)C[C@@H]43)cc2C)c(O)c1] | 355 0.77 U | 1H NMR (400 MHz, DMSO-d6, 30° C.) d ppm 1.76- 1.92 (m, 2 H), 2.05-2.15 (m, 1 H), 2.11 (s, 3 H), 2.18 (s, 3 H), 2.28 (s, 3 H), 2.30-2.36 (m, 1 H), 2.51-2.55 (m partially hidden, 2 H), 3.14 (td, J = 3.8 and 12.7 Hz, 1 H), 3.61 (td, J = 3.3 and 11.8 Hz, 1 H), 3.72 (br q, J = 2.8 Hz, 1 H), 3.90 (br d, J = 12.7 Hz, 1 H), 4.04 (dd, J = 3.3 and 11.8 Hz, 1 H), 4.36- 4.44 (m, 1 H), 6.70 (br d, J = 7.6 Hz, 1 H), 6.73 (br s, 1 H), 7.04-7.08 (m, 2 H), 9.64 (br s, 1 H) |
| 374 | 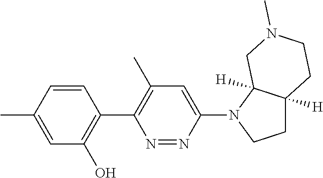 [SMILES: Cc1ccc(-c2nnc(N3CC[C@@H]4CCN(C)C[C@@H]43)cc2C)c(O)c1] | 339 0.67 Z1 | 1H NMR (400 MHz, DMSO-d6, 30° C.) d ppm 1.66- 1.77 (m, 2 H), 1.85-2.06 (m, 3 H), 2.07-2.14 (m, 1 H), 2.09 (s, 3 H), 2.17 (s, 3 H), 2.28 (s, 3 H), 2.29- 2.38 (m, 1 H), 2.52-2.57 (m partially hidden, 1 H), 3.06 (br dd, J = 6.0 and 10.5 Hz, 1 H), 3.34-3.41 (m, 1 H), 3.49-3.57 (m, 1 H), 4.15-4.23 (m, 1 H), 6.69 (dd, J = 1.3 and 7.8 Hz, 1 H), 6.73 (br s, 2 H), 7.05 (d, J = 7.8 Hz, 1 H), 9.70 (br s, 1 H) |
| 375 | 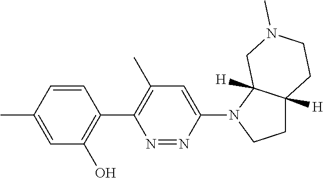 [SMILES: Cc1ccc(-c2nnc(N3CC[C@H]4CCN(C)C[C@H]43)cc2C)c(O)c1] | 339 0.67 Z1 | 1H NMR (400 MHz, DMSO-d6, 30° C.) d ppm 1.66- 1.78 (m, 2 H), 1.85-2.04 (m, 3 H), 2.05-2.13 (m, 1 H), 2.09 (s, 3 H), 2.17 (s, 3 H), 2.28 (s, 3 H), 2.30- 2.38 (m, 1 H), 2.52-2.56 (m partially hidden, 1 H), 3.06 (br dd, J = 6.0 and 10.5 Hz, 1 H), 3.34-3.42 (m, 1 H), 3.49-3.57 (m, 1 H), 4.15-4.25 (m, 1 H), 6.69 (br d, J = 7.8 Hz, 1 H), 6.73 (br s, 2 H), 7.05 (d, J = 7.8 Hz, 1 H), 9.70 (br s, 1 H) |
| 376 | 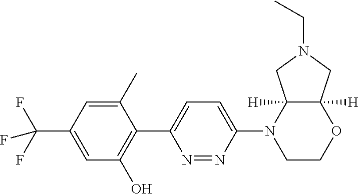 [SMILES: CCN1C[C@H]2OCCN(c3ccc(-c4c(C)cc(C(F)(F)F)cc4O)nn3)[C@H]2C1] | 409.5 2.58 E | ¹H NMR (400 MHz, DMSO-d₆) δ (ppm): 10.24 (s, 1H), 7.47 (d, J = 9.3 Hz, 1H), 7.41 (d, J = 9.3 Hz, 1H), 7.12 (s, 1H), 7.06 (s, 1H), 4.14-4.04 (m, 1H), 3.93 (d, J = 12.5 Hz, 1H), 3.88-3.81 (m, 1H), 3.79- 3.68 (m, 2H), 3.26-3.12 (m, 1H), 3.06-2.90 (m, 2H), 2.80 (t, J = 9.9 Hz, 1H), 2.70-2.60 (m, 3H), 2.11 (s, 3H), 1.07-0.97 (m, 3H) |
| 377 | 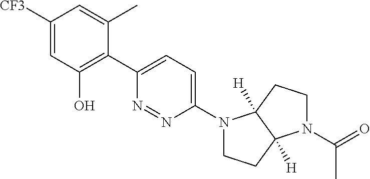 [SMILES: CC(=O)N1CC[C@H]2[C@@H]1CCN2c1ccc(-c2c(C)cc(C(F)(F)F)cc2O)nn1] | 407 1.09 B | 1H NMR (400 MHz, DMSO-d₆, 100° C.) δ ppm 1.93- 2.28 (m, 4 H) 2.00 (br s, 3 H) 2.17 (s, 3 H) 3.19- 3.59 (m, 2 H) 3.61-3.78 (m, 2 H) 4.56-4.64 (m, 1 H) 4.64-4.79 (m, 1 H) 6.97 (d, J = 9.3 Hz, 1 H) 7.06 (br s, 2 H) 7.36 (d, J = 9.3 Hz, 1 H) 9.51-10.13 (m, 1 H) |
| 378 | 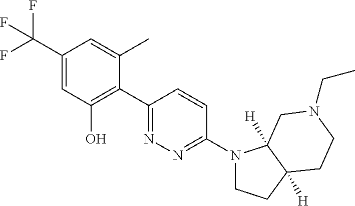 [SMILES: CCN1CC[C@H]2CCN(c3ccc(-c4c(C)cc(C(F)(F)F)cc4O)nn3)[C@H]2C1] | 407.5 2.71 E | ¹H NMR (400 MHz, DMSO-d6) δ (ppm): 10.16 (s, 1H), 7.31 (d, J = 9.3 Hz, 1H), 7.11 (s, 1H), 7.05 (d, J = 1.8 Hz, 1H), 6.91 (d, J = 9.3 Hz, 1H), 4.29- 4.13 (m, 1H), 3.56 (t, J = 9.6 Hz, 1H), 3.49-3.37 (m, 1H), 3.22-3.07 (m, 1H), 2.76-2.65 (m, 1H), 2.45-2.27 (m, 3H), 2.20-2.00 (m, 5H), 1.97- 1.84 (m, 2H), 1.80 (t, J = 10.6 Hz, 1H), 1.74-1.63 (m, 1H), 0.99 (t, J = 7.1 Hz, 3H). |
| 379 | 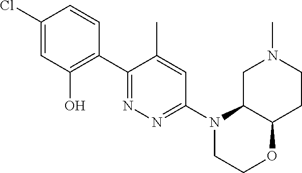 [SMILES: Cc1cc(N2CCO[C@@H]3CCN(C)C[C@@H]32)nnc1-c1ccc(Cl)cc1O] | 375 0.86 U | 1H NMR (400 MHz, DMSO-d6, 30° C.) d ppm 1.75- 1.93 (m, 2 H), 2.04-2.14 (m, 1 H), 2.09 (s, 3 H), 2.17 (s, 3 H), 2.27-2.35 (m, 1 H), 2.51-2.55 (m partially hidden, 2 H), 3.14 (td, J = 3.8 and 12.7 Hz, 1 H), 3.61 (td, J = 3.3 and 11.8 Hz, 1 H), 3.72 (br q, J = 2.8 Hz, 1 H), 3.91 (br d, J = 12.7 Hz, 1 H), 4.04 (dd, J = 3.3 and 11.8 Hz, 1 H), 4.35-4.45 (m, 1 H), 6.93-6.97 (m, 2 H), 7.09 (br s, 1 H), 7.20 (br d, J = 7.8 Hz, 1 H), 10.18-10.29 (m, 1 H) |
| 380 | 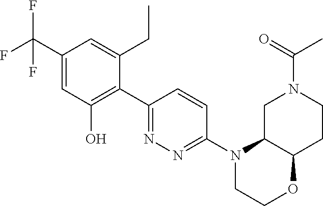 [SMILES: CCc1cc(C(F)(F)F)cc(O)c1-c1ccc(N2CCO[C@@H]3CCN(C(C)=O)C[C@@H]32)nn1] | 451 1.36 U | 1H NMR (400 MHz, DMSO-d6, 100° C.) d ppm 1.03 (t, J = 7.5 Hz, 3 H), 1.78-1.94 (m, 2 H), 2.04 (s, 3 H), 2.51-2.54 (m partially hidden, 2 H), 2.72- 3.17 (m partially hidden, 2 H), 3.28 (br td, J = 3.8 and 12.6 Hz, 1 H), 3.50-4.31 (m partially hidden, 2 H), 3.73 (td, J = 3.2 and 11.8 Hz, 1 H), 3.91 (q, J = 2.8 Hz, 1 H), 4.02 (br d, J = 12.6 Hz, 1 H), 4.12 (br dd, J = 3.8 and 11.8 Hz, 1 H), 4.25-4.48 (m, 1 H), 7.08-7.11 (m, 2 H), 7.25 (br d, J = 9.5 Hz, 1 H), 7.41 (d, J = 9.5 Hz, 1 H), 9.07-10.18 (m, 1 H) |
| 381 | 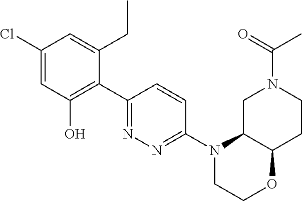 [SMILES: CCc1cc(Cl)cc(O)c1-c1ccc(N2CCO[C@@H]3CCN(C(C)=O)C[C@@H]32)nn1] | 417 1.23 U | 1H NMR (400 MHz, DMSO-d6, 100° C.) d ppm 1.00 (t, J = 7.5 Hz, 3 H), 1.78-1.94 (m, 2 H), 2.03 (s, 3 H), 2.43 (q, J = 7.5 Hz, 2 H), 2.72-3.20 (m partially hidden, 2 H), 3.27 (td, J = 3.8 and 12.6 Hz, 1 H), 3.48-4.25 (m partially hidden, 2 H), 3.73 (td, J = 3.2 and 11.8 Hz, 1 H), 3.90 (q, J = 2.8 Hz, 1 H), 4.00 (br d, J = 12.6 Hz, 1 H), 4.11 (dd, J = 3.8 and 11.8 Hz, 1 H), 4.26-4.43 (m, 1 H), 6.82-6.86 (m, 2 H), 7.22 (br d, J = 9.5 Hz, 1 H), 7.36 (d, J = 9.5 Hz, 1 H), 8.99-9.90 (m, 1 H) |
| 382 | 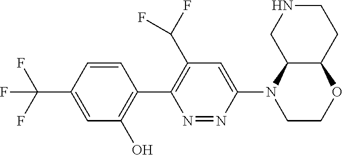 [SMILES: Oc1cc(C(F)(F)F)ccc1-c1nnc(N2CCO[C@@H]3CCNC[C@@H]32)cc1C(F)F] | 431 1.00 U | 1H NMR (400 MHz, DMSO-d6, 30° C.) d ppm 1.74- 1.82 (m, 2 H), 2.63-2.69 (m, 3 H), 2.92-3.00 (m, 1 H), 3.17-3.24 (m partially hidden, 1 H), 3.58- 3.66 (m, 1 H), 3.84 (br q, J = 2.8 Hz, 1 H), 4.03- 4.13 (m, 2 H), 4.29-4.40 (m, 1 H), 6.84 (t, J = 54.8 Hz, 1 H), 7.21 (br s, 1 H), 7.24 (br d, J = 8.0 Hz, 1 H), 7.34 (s, 1 H), 7.49 (d, J = 8.0 Hz, 1 H) |
| 383 | 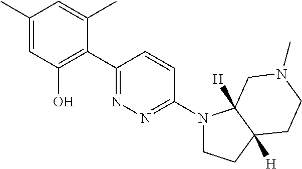 [SMILES: Cc1cc(C)c(-c2ccc(N3CC[C@H]4CCN(C)C[C@H]43)nn2)c(O)c1] | 339 0.74 U | 1H NMR (400 MHz, DMSO-d6, 30° C.) d ppm 1.66- 1.80 (m, 2 H), 1.85-2.02 (m, 3 H), 2.04 (s, 3 H), 2.05-2.15 (m, 1 H), 2.16 (s, 3 H), 2.21 (s, 3 H), 2.28-2.40 (m, 1 H), 2.53-2.54 (m partially hidden, 1 H), 3.05 (br dd, J = 6.0 and 10.5 Hz, 1 H), 3.34-3.43 (m, 1 H), 3.49-3.60 (m, 1 H), 4.15- 4.25 (m, 1 H), 6.55 (s, 1 H), 6.56 (s, 1 H), 6.86 (d, J = 9.5 Hz, 1 H), 7.24 (d, J = 9.5 Hz, 1 H), 9.39 (br s, 1 H) |
| 384 | 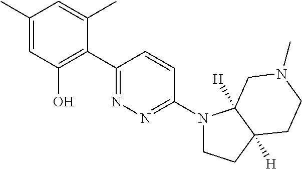 [SMILES: Cc1cc(C)c(-c2ccc(N3CC[C@@H]4CCN(C)C[C@@H]43)nn2)c(O)c1] | 339 0.75 U | 1H NMR (400 MHz, DMSO-d6, 30° C.) d ppm 1.66- 1.80 (m, 2 H), 1.85-2.02 (m, 3 H), 2.04 (s, 3 H), 2.06-2.13 (m, 1 H), 2.17 (s, 3 H), 2.21 (s, 3 H), 2.31-2.41 (m, 1 H), 2.53-2.56 (m partially hidden, 1 H), 3.05 (br dd, J = 6.0 and 10.5 Hz, 1 H), 3.35-3.43 (m, 1 H), 3.50-3.59 (m, 1 H), 4.15- 4.26 (m, 1 H), 6.56 (br s, 1 H), 6.57 (br s, 1 H), 6.87 (d, J = 9.5 Hz, 1 H), 7.24 (d, J = 9.5 Hz, 1 H), 9.39 (br s, 1 H) |
| 385 | 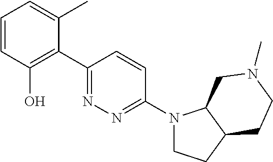 [SMILES: Cc1cccc(O)c1-c1ccc(N2CC[C@@H]3CCN(C)C[C@@H]32)nn1] | 325 0.66 U | 1H NMR (400 MHz, DMSO-d6, 30° C.) d ppm 1.66- 1.81 (m, 2 H), 1.85-2.05 (m, 3 H), 2.04-2.14 (m, 1 H), 2.07 (s, 3 H), 2.17 (s, 3 H), 2.34-2.41 (m, 1 H), 2.53-2.56 (m partially hidden, 1 H), 3.06 (br dd, J = 6.0 and 10.5 Hz, 1 H), 3.37-3.43 (m, 1 H), 3.52-3.60 (m, 1 H), 4.17-4.26 (m, 1 H), 6.71- 6.78 (m, 2 H), 6.88 (d, J = 9.5 Hz, 1 H), 7.08 (t, J = 8.0 Hz, 1 H), 7.27 (d, J = 9.5 Hz, 1 H), 9.43 (br s, 1 H) |
| 386 | 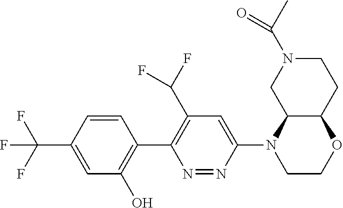 [SMILES: CC(=O)N1CC[C@H]2OCCN(c3cc(C(F)F)c(-c4ccc(C(F)(F)F)cc4O)nn3)[C@H]2C1] | 473 1.4 U | 1H NMR (400 MHz, DMSO-d6, 101º C.) d ppm 1.81-1.90 (m, 2 H), 2.02 (s, 3 H), 2.66-3.27 (m partially hidden, 2 H), 3.32 (td, J = 3.8 and 12.6 Hz, 1 H), 3.51-4.34 (m partially hidden, 2 H), 3.72 (td, J = 3.2 and 11.8 Hz, 1 H), 3.89 (br q, J = 2.8 Hz, 1 H), 4.05-4.13 (m, 2 H), 4.37-4.50 (m, 1 H), 6.78 (t, J = 54.5 Hz, 1 H), 7.18-7.23 (m, 2 H), 7.36 (br s, 1 H), 7.48 (d, J = 7.8 Hz, 1 H) |
| 387 | 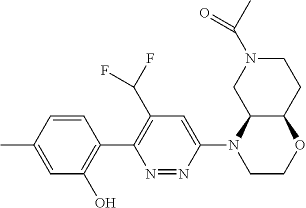 [SMILES: CC(=O)N1CC[C@H]2OCCN(c3cc(C(F)F)c(-c4ccc(C)cc4O)nn3)[C@H]2C1] | 419 1.27 U | 1H NMR (400 MHz, DMSO-d6, 101° C.) d ppm 1.81-1.89 (m, 2 H), 2.01 (s, 3 H), 2.30 (s, 3 H), 2.68-3.19 (m partially hidden, 2 H), 3.30 (td, J = 3.8 and 12.6 Hz, 1 H), 3.53-4.28 (m partially hidden, 2 H), 3.71 (td, J = 3.2 and 11.8 Hz, 1 H), 3.89 (br q, J = 2.8 Hz, 1 H), 4.05 (br d, J = 12.6 Hz, 1 H), 4.10 (dd, J = 3.8 and 11.8 Hz, 1 H), 4.31-4.49 (m, 1 H), 6.66 (br t, J = 54.7 Hz, 1 H), 6.73-6.80 (m, 2 H), 7.15 (d, J = 7.6 Hz, 1 H), 7.32 (br s, 1 H) |
| 388 | 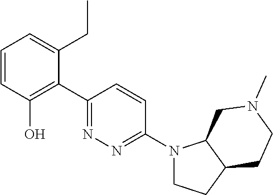 [SMILES: CCc1cccc(O)c1-c1ccc(N2CC[C@@H]3CCN(C)C[C@@H]32)nn1] | 379 0.84 U | 1H NMR (400 MHz, DMSO-d6, 30° C.) d ppm 0.95 (t, J = 7.5 Hz, 3 H), 1.67-1.82 (m, 2 H), 1.87-2.14 (m, 4 H), 2.18 (s, 3 H), 2.34-2.44 (m, 3 H), 2.53- 2.57 (m partially hidden, 1 H), 3.07 (br dd, J = 6.0 and 10.5 Hz, 1 H), 3.36-3.46 (m, 1 H), 3.52-3.61 (m, 1 H), 4.17-4.26 (m, 1 H), 6.75 (d, J = 7.8 Hz, 2 H), 6.87 (d, J = 9.5 Hz, 1 H), 7.12 (t, J = 7.8 Hz, 1 H), 7.23 (d, J = 9.5 Hz, 1 H), 9.28 (br s, 1 H) |
| 389 | 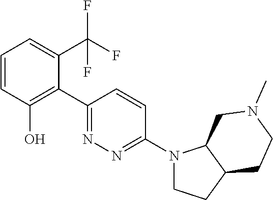 [SMILES: CN1CC[C@H]2CCN(c3ccc(-c4c(O)cccc4C(F)(F)F)nn3)[C@H]2C1] | 379 0.84 U | 1H NMR (400 MHz, DMSO-d6, 30° C.) d ppm 1.68- 1.86 (m, 2 H), 1.86-2.01 (m, 2 H), 2.04-2.16 (m, 2 H), 2.20 (s, 3 H), 2.34-2.42 (m, 1 H), 2.54-2.60 (m partially hidden, 1 H), 3.04-3.13 (m, 1 H), 3.36- 3.46 (m, 1 H), 3.52-3.61 (m, 1 H), 4.16-4.29 (m, 1 H), 6.88 (d, J = 9.5 Hz, 1 H), 7.20-7.27 (m, 3 H), 7.43 (t, J = 7.8 Hz, 1 H), 10.02 (br s, 1 H) |
| 390 | 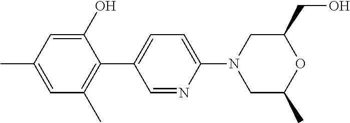 [SMILES: Cc1cc(C)c(-c2ccc(N3C[C@@H](CO)O[C@@H](C)C3)nc2)c(O)c1] | 330 0.95 B | 1H NMR (400 MHz, DMSO-d₆, 30° C.) δ ppm 1.20 (d, J = 6.0 Hz, 3 H) 2.03 (s, 3 H) 2.22 (s, 3 H) 2.54- 2.65 (m, 2 H) 3.40-3.50 (m, 1 H) 3.50-3.62 (m, 2 H) 3.64-3.73 (m, 1 H) 4.24 (br d, J = 12.5 Hz, 1 H) 4.35 (br d, J = 12.5 Hz, 1 H) 4.79 (t, J = 5.6 Hz, 1 H) 6.57 (br s, 1 H) 6.58 (br s, 1 H) 7.26 (d, J = 9.5 Hz, 1 H) 7.34 (d, J = 9.5 Hz, 1 H) 9.37 (s, 1 H) |
| 391 | 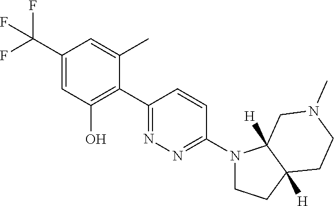 [SMILES: Cc1cc(C(F)(F)F)cc(O)c1-c1ccc(N2CC[C@H]3CCN(C)C[C@H]32)nn1] | 393.5 2.66 E | ¹H NMR (500 MHz, DMSO-d₆) δ (ppm): 10.06 (s, 1H), 7.24 (d, J = 9.3 Hz, 1H), 7.04 (s, 1H), 6.99 (d, J = 1.8 Hz, 1H), 6.85 (d, J = 9.3 Hz, 1H), 4.16 (s, 1H), 3.54-3.46 (m, 1H), 3.43-3.30 (m, 1H), 3.04- 2.98 (m, 1H), 2.52-2.45 (m, 1H), 2.37-2.30 (m, 1H), 2.11 (s, 3H), 2.07 (s, 3H), 2.05-2.00 (m, 1H), 2.01-1.94 (m, 1H), 1.93-1.80 (m, 2H), 1.75- 1.67 (m, 1H), 1.67-1.60 (m, 1H). |
| 392 | 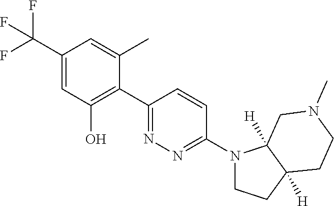 [SMILES: Cc1cc(C(F)(F)F)cc(O)c1-c1ccc(N2CC[C@@H]3CCN(C)C[C@@H]32)nn1] | 393.5 2.66 E | ¹H NMR (400 MHz, DMSO-d₆) δ (ppm): 10.15 (s, 1H), 7.31 (d, J = 9.3 Hz, 1H), 7.10 (s, 1H), 7.05 (d, J = 1.8 Hz, 1H), 6.92 (d, J = 9.3 Hz, 1H), 4.23 (s, 1H), 3.62-3.52 (m, 1H), 3.43-3.40 (m, 1H), 3.12- 3.03 (m, 1H), 2.59-2.52 (m, 1H), 2.43-2.34 (m, 1H), 2.18 (s, 3H), 2.14 (s, 3H), 2.12-2.06 (m, 1H), 2.05-2.00 (m, 1H), 2.00-1.85 (m, 2H), 1.82- 1.66 (m, 2H). |
| 393 | 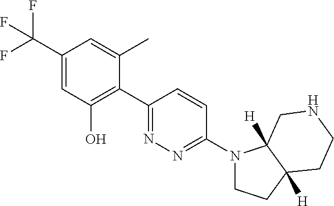 [SMILES: Cc1cc(C(F)(F)F)cc(O)c1-c1ccc(N2CC[C@H]3CCNC[C@H]32)nn1] | 379.4 2.47 E | ¹H NMR (500 MHz, DMSO-d₆) δ (ppm): 9.80 (s, 1H), 7.24 (d, J = 9.3 Hz, 1H), 7.04 (s, 1H), 6.98 (s, 1H), 6.83 (d, J = 9.3 Hz, 1H), 4.02 (s, 1H), 3.51 (t, J = 9.4 Hz, 1H), 3.38-3.31 (m, 2H), 2.70-2.65 (m, 1H), 2.60-2.51 (m, 1H), 2.39-2.26 (m, 2H), 2.18-2.09 (m, 1H), 2.07 (s, 3H), 1.90-1.73 (m, 2H), 1.63-1.56 (m, 1H) (NH not visible) |
| 394 | 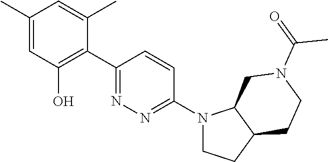 [SMILES: CC(=O)N1CC[C@H]2CCN(c3ccc(-c4c(C)cc(C)cc4O)nn3)[C@H]2C1] | 367 0.93 U | 1H NMR (400 MHz, DMSO-d6, 100° C.) d ppm 1.63-1.78 (m, 1 H), 1.87-1.99 (m, 1 H), 1.96 (br s, 3 H), 2.05-2.09 (m, 2 H), 2.10 (s, 3 H), 2.25 (s, 3 H), 2.54-2.60 (m partially hidden, 1 H), 3.28-3.39 (m, 1 H), 3.41-3.52 (m, 1 H), 3.59-3.69 (m, 2 H), 3.78-4.66 (m, 2 H), 4.16-4.26 (m, 1 H), 6.59 (br s, 1 H), 6.60 (br s, 1 H), 6.91 (br d, J = 9.5 Hz, 1 H), 7.34 (d, J = 9.5 Hz, 1 H), 9.13-9.25 (m, 1 H) |
| 395 | 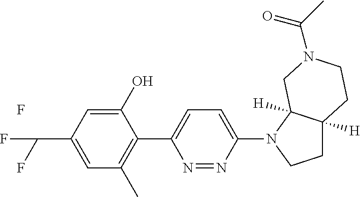 | 421 1.12 U | 1H NMR (400 MHz, DMSO-d6, 100° C.) d ppm 1.65-1.79 (m, 1 H), 1.90-2.00 (m, 1 H), 1.96 (br s, 3 H), 2.02-2.14 (m, 2 H), 2.18 (s, 3 H), 2.54- 2.61 (m partially hidden, 1 H), 3.27-3.38 (m, 1 H), 3.46-3.54 (m, 1 H), 3.60-3.69 (m, 2 H), 3.82 - 4.69 (m, 2 H), 4.17-4.29 (m, 1 H), 6.94 (br d, J = 9.3 Hz, 1 H), 7.07-7.10 (m, 2 H), 7.37 (d, J = 9.3 Hz, 1 H), 9.35-10.16 (m, 1 H) |
| 397 | 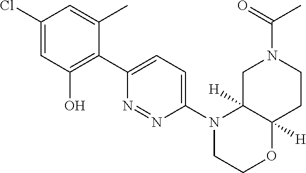 [SMILES: CC(=O)N1CC[C@H]2OCCN(c3ccc(-c4c(C)cc(Cl)cc4O)nn3)[C@H]2C1] | 403 1.18 U | 1H NMR (400 MHz, DMSO-d₆, 100° C.) δ ppm 1.73- 1.91 (m, 2 H) 2.01 (s, 3 H) 2.08 (s, 3 H) 2.73- 3.16 (m, 4 H) 3.25 (br td, J = 12.6, 3.8 Hz, 1 H) 3.70 (br td, J = 11.8, 3.2 Hz, 1 H) 3.86-3.90 (m, 1 H) 3.99 (br d, J = 12.6 Hz, 1 H) 4.09 (br dd, J = 11.8, 3.8 Hz, 1 H) 4.28-4.39 (m, 1 H) 6.80-6.83 (m, 2 H) 7.21 (br d, J = 9.5 Hz, 1 H) 7.37 (d, J = 9.5 Hz, 1 H) 9.16-10.03 (m, 1 H) |
| 398 | 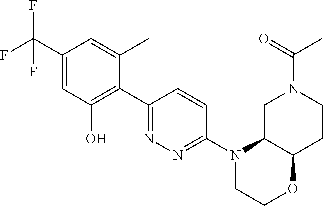 [SMILES: CC(=O)N1CC[C@H]2OCCN(c3ccc(-c4c(C)cc(C(F)(F)F)cc4O)nn3)[C@H]2C1] | 437 1.36 U | 1H NMR (400 MHz, DMSO-d₆, 100° C.) δ ppm 1.79- 1.89 (m, 2 H) 2.02 (s, 3 H) 2.16 (s, 3 H) 2.72- 4.57 (m, 4 H) 3.26 (td, J = 12.7, 3.8 Hz, 1 H) 3.71 (td, J = 11.8, 3.2 Hz, 1 H) 3.83-3.94 (m, 1 H) 4.01 (br d, J = 11.8 Hz, 1 H) 4.10 (dd, J = 11.8, 3.2 Hz, 1 H) 4.26-4.42 (m, 1 H) 7.06-7.09 (m, 2 H) 7.24 (br d, J = 9.5 Hz, 1 H) 7.41 (d, J = 9.5 Hz, 1 H) 9.65- 9.97 (m, 1 H) |
| 399 | 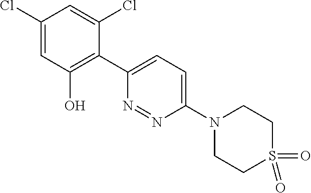 [SMILES: O=S1(=O)CCN(c2ccc(-c3c(O)cc(Cl)cc3Cl)nn2)CC1] | 374 1.28 U | 1H NMR (500 MHz, DMSO-d₆, 27° C.) δ ppm 3.20- 3.25 (m, 4 H) 4.16-4.22 (m, 4 H) 6.97 (d, J = 2.0 Hz, 1 H) 7.15 (d, J = 2.0 Hz, 1 H) 7.46 (d, J = 9.5 Hz, 1 H) 7.50 (d, J = 9.5 Hz, 1 H) 10.49-10.66 (m, 1 H) |
| 400 | 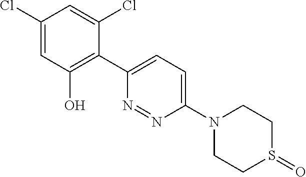 [SMILES: O=S1CCN(c2ccc(-c3c(O)cc(Cl)cc3Cl)nn2)CC1] | 358 1.16 U | 1H NMR (500 MHz, DMSO-d₆, 27° C.) δ ppm 2.76 (dt, J = 13.9, 2.0 Hz, 2 H) 2.97 (ddd, J = 13.9, 11.2, 3.4 Hz, 2 H) 4.00 (ddd, J = 14.8, 11.2, 2.0 Hz, 2 H) 4.30 (dt, J = 14.8, 3.4 Hz, 2 H) 6.97 (d, J = 2.1 Hz, 1 H) 7.14 (d, J = 2.1 Hz, 1 H) 7.42 (d, J = 9.5 Hz, 1 H) 7.45 (d, J = 9.5 Hz, 1 H) 10.49-10.72 (m, 1 H) |
| 402 | 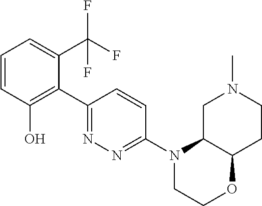 [SMILES: CN1CC[C@H]2OCCN(c3ccc(-c4c(O)cccc4C(F)(F)F)nn3)[C@H]2C1] | 395 0.86 U | 1H NMR (400 MHz, DMSO-d6, 30° C.) d ppm 1.76- 1.94 (m, 2 H), 2.05-2.15 (m, 1 H), 2.18 (s, 3 H), 2.30-2.39 (m, 1 H), 2.52-2.55 (m hidden, 2 H), 3.17 (td, J = 3.8 and 12.7 Hz, 1 H), 3.64 (td, J = 3.3 and 11.8 Hz, 1 H), 3.75 (br q, J = 2.8 Hz, 1 H), 3.94 (br d, J = 12.7 Hz, 1 H), 4.05 (br dd, J = 3.3 and 11.8 Hz, 1 H), 4.37-4.47 (m, 1 H), 7.17-7.29 (m, 3 H), 7.35 (d, J = 9.5 Hz, 1 H), 7.42-7.48 (m, 1 H), 10.01- 10.13 (m, 1 H) |
| 403 | 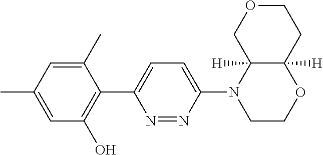 [SMILES: Cc1cc(C)c(-c2ccc(N3CCO[C@@H]4CCOC[C@@H]43)nn2)c(O)c1] | 342 1.03 U | 1H NMR (400 MHz, DMSO-d₆, 30° C.) δ ppm 1.74- 1.81 (m, 1 H) 1.91-2.00 (m, 1 H) 2.03 (s, 3 H) 2.22 (s, 3 H) 3.16 (td, J = 12.7, 3.8 Hz, 1 H) 3.47-3.77 (m, 5 H) 3.86-3.97 (m, 2 H) 4.08 (dd, J = 11.8, 3.3 Hz, 1 H) 4.41-4.49 (m, 1 H) 6.57 (br s, 1 H) 6.58 (br s, 1 H) 7.23 (d, J = 9.5 Hz, 1 H) 7.33 (d, J = 9.5 Hz, 1 H) 9.35 (br s, 1 H) |
| 404 | 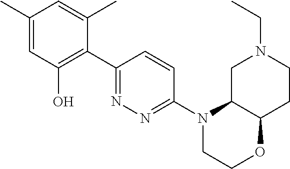 [SMILES: CCN1CC[C@H]2OCCN(c3ccc(-c4c(C)cc(C)cc4O)nn3)[C@H]2C1] | 369 0.82 U | 1H NMR (400 MHz, DMSO-d₆, 30° C.) δ ppm 0.97 (t, J = 7.0 Hz, 3 H) 1.75-1.90 (m, 2 H) 2.03 (s, 3 H) 2.05-2.13 (m, 1 H) 2.22 (s, 3 H) 2.30-2.41 (m, 3 H) 2.56-2.65 (m, 2 H) 3.16 (td, J = 12.7, 3.8 Hz, 1 H) 3.63 (td, J = 11.8, 3.3 Hz, 1 H) 3.75 (br q, J = 2.8 Hz, 1 H) 3.93 (br d, J = 12.7 Hz, 1 H) 4.04 (dd, J = 11.8, 3.3 Hz, 1 H) 4.33-4.45 (m, 1 H) 6.57 (s, 1 H) 6.58 (s, 1 H) 7.19 (d, J = 9.5 Hz, 1 H) 7.34 (d, J = 9.5 Hz, 1 H) 9.36 (s, 1 H) |
| 405 | 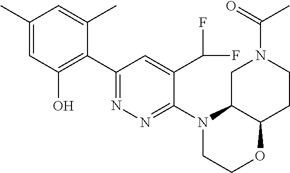 [SMILES: CC(=O)N1CC[C@H]2OCCN(c3nnc(-c4c(C)cc(C)cc4O)cc3C(F)F)[C@H]2C1] | 433 1.36 U | 1H NMR (400 MHz, DMSO-d6, 100° C.) d ppm 1.70-1.82 (m, 1 H), 1.83-1.93 (m, 1 H), 1.96 (s, 3 H), 2.08 (s, 3 H), 2.27 (s, 3 H), 2.72-3.23 (m partially hidden, 2 H), 3.32-3.40 (m, 1 H), 3.46- 4.27 (m partially hidden, 2 H), 3.63-3.71 (m, 1 H), 3.76-3.88 (m, 2 H), 4.00-4.10 (m, 2 H), 6.64 (br s, 1 H), 6.65 (br s, 1 H), 7.15 (t, J = 53.7 Hz, 1 H), 7.64 (s, 1 H), 9.09 (br s, 1 H) |
| 406 | 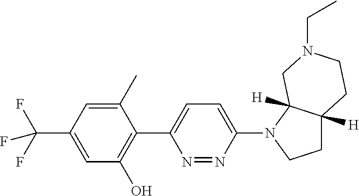 [SMILES: CCN1CC[C@@H]2CCN(c3ccc(-c4c(C)cc(C(F)(F)F)cc4O)nn3)[C@@H]2C1] | 407.5 2.71 E | ¹H NMR (400 MHz, DMSO-d6) δ (ppm): 10.16 (s, 1H), 7.31 (d, J = 9.3 Hz, 1H), 7.11 (s, 1H), 7.05 (d, J = 1.8 Hz, 1H), 6.91 (d, J = 9.3 Hz, 1H), 4.29- 4.13 (m, 1H), 3.56 (t, J = 9.6 Hz, 1H), 3.49-3.37 (m, 1H), 3.22-3.07 (m, 1H), 2.76-2.65 (m, 1H), 2.45-2.27 (m, 3H), 2.20-2.00 (m, 5H), 1.97- 1.84 (m, 2H), 1.80 (t, J = 10.6 Hz, 1H), 1.74-1.63 (m, 1H), 0.99 (t, J = 7.1 Hz, 3H). |
| 407 | 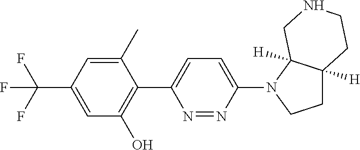 [SMILES: Cc1cc(C(F)(F)F)cc(O)c1-c1ccc(N2CC[C@@H]3CCNC[C@@H]32)nn1] | 379.5 2.44 E | ¹H NMR (500 MHz, CDCl₃) δ (ppm): δ 7.46 (d, J = 9.6 Hz, 1H), 7.17 (d, J = 1.7 Hz, 1H), 7.03 (s, 1H), 6.80 (d, J = 9.5 Hz, 1H), 4.20 (s, 1H), 3.72 (t, J = 9.7 Hz, 1H), 3.59-3.50 (m, 1H), 3.44 (s, 1H), 2.89 (d, J = 12.6 Hz, 1H), 2.77 (td, J = 12.3, 3.2 Hz, 1H), 2.65-2.50 (m, 2H), 2.45 (s, 3H), 2.33-2.19 (m, 1H), 2.06-1.92 (m, 2H), 1.77 (d, J = 14.2 Hz, 1H) (NH and OH not visible). |
| 408 | 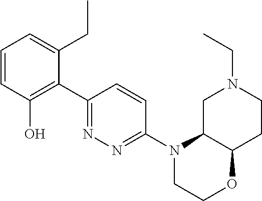 [SMILES: CCc1cccc(O)c1-c1ccc(N2CCO[C@@H]3CCN(CC)C[C@@H]32)nn1] | 368 0.82 U | 1H NMR (400 MHz, DMSO-d₆, 30° C.) δ ppm 0.92- 1.01 (m, 6 H) 1.75-1.91 (m, 2 H) 2.10 (td, J = 11.4, 3.8 Hz, 1 H) 2.30-2.43 (m, 5 H) 2.56-2.65 (m, 2 H) 3.17 (td, J = 12.7, 3.8 Hz, 1 H) 3.63 (td, J = 11.8, 3.3 Hz, 1 H) 3.76 (br q, J = 2.8 Hz, 1 H) 3.94 (br d, J = 12.7 Hz, 1 H) 4.05 (dd, J = 11.8, 3.3 Hz, 1 H) 4.35-4.44 (m, 1 H) 6.77 (d, J = 7.8 Hz, 2 H) 7.14 (t, J = 7.8 Hz, 1 H) 7.20 (d, J = 9.5 Hz, 1 H) 7.33 (d, J = 9.5 Hz, 1 H) 9.32 (s, 1 H) |
| 409 | 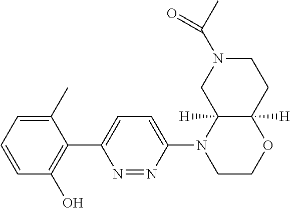 [SMILES: CC(=O)N1CC[C@H]2OCCN(c3ccc(-c4c(C)cccc4O)nn3)[C@H]2C1] | 369 0.95 U | 1H NMR (400 MHz, DMSO-d6, 100° C.) d ppm 1.82-1.91 (m, 2 H), 2.04 (s, 3 H), 2.12 (s, 3 H), 2.69-3.21 (m partially hidden, 2 H), 3.27 (td, J = 3.8 and 12.6 Hz, 1 H), 3.60-4.45 (m partially hidden, 2 H), 3.73 (td, J = 3.2 and 11.8 Hz, 1 H), 3.91 (br q, J = 2.8 Hz, 1 H), 4.01 (br d, J = 12.6 Hz, 1 H), 4.11 (dd, J = 3.8 and 11.8 Hz, 1 H), 4.29-4.43 (m, 1 H), 6.75-6.81 (m, 2 H), 7.11 (t, J = 7.8 Hz, 1 H), 7.23 (br d, J = 9.5 Hz, 1 H), 7.40 (d, J = 9.5 Hz, 1 H), 9.11-9.21 (m, 1 H) |
| 410 | 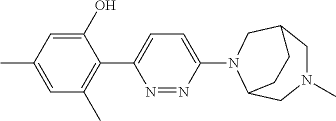 | 339 0.71 and 0.72 B | 1H NMR (400 MHz, DMSO-d₆, 30° C.) δ ppm 1.56- 1.66 (m, 1 H) 1.79-1.90 (m, 1 H) 1.93-2.09 (m, 2 H) 2.05 (s, 3 H) 2.22 (s, 3 H) 2.25 (s, 3 H) 2.26- 2.34 (m, 3 H) 2.92-3.00 (m, 2 H) 3.40 (br dd, J = 11.5, 3.0 Hz, 1 H) 3.51 (br d, J = 11.5 Hz, 1 H) 4.73-4.87 (m, 1 H) 6.56 (br s, 1 H) 6.57 (br s, 1 H) 6.96 (d, J = 9.5 Hz, 1 H) 7.28 (d, J = 9.5 Hz, 1 H) 9.42 (br s, 1 H) |
| 411 | 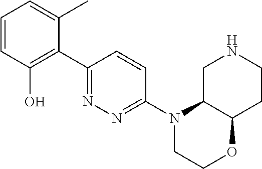 [SMILES: Cc1cccc(O)c1-c1ccc(N2CCO[C@@H]3CCNC[C@@H]32)nn1] | 327 0.69 and 0.70 U | 1H NMR (400 MHz, DMSO-d₆, 30° C.) δ ppm 1.75- 1.87 (m, 2 H) 2.06 (s, 3 H) 2.64-2.78 (m, 3 H) 2.99 (t, J = 11.8 Hz, 1 H) 3.16 (td, J = 12.6, 3.8 Hz, 2 H) 3.63 (td, J = 11.8, 3.2 Hz, 1 H) 3.85 (br q, J = 2.6 Hz, 1 H) 3.95 (br d, J = 12.6 Hz, 1 H) 4.06 (dd, J = 11.8, 3.8 Hz, 1 H) 4.31-4.38 (m, 1 H) 6.72-6.78 (m, 2 H) 7.07-7.12 (m, 1 H) 7.22 (d, J = 9.5 Hz, 1 H) 7.36 (d, J = 9.5 Hz, 1 H) 9.37-9.48 (m, 1 H) |
| 412 | 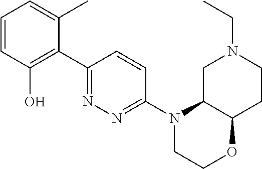 [SMILES: CCN1CC[C@H]2OCCN(c3ccc(-c4c(C)cccc4O)nn3)[C@H]2C1] | 355 0.73 U | 1H NMR (500 MHz, DMSO-d₆, 27° C.) δ ppm 0.97 (t, J = 7.0 Hz, 3 H) 1.76-1.91 (m, 2 H) 2.04-2.12 (m, 1 H) 2.06 (s, 3 H) 2.31-2.41 (m, 3 H) 2.58- 2.64 (m, 2 H) 3.16 (td, J = 12.7, 3.8 Hz, 1 H) 3.63 (td, J = 11.8, 3.3 Hz, 1 H) 3.76 (q, J = 2.8 Hz, 1 H) 3.94 (br d, J = 12.7 Hz, 1 H) 4.05 (dd, J = 11.8, 3.3 Hz, 1 H) 4.36-4.44 (m, 1 H) 6.73-6.78 (m, 2 H) 7.10 (t, J = 7.8 Hz, 1 H) 7.21 (d, J = 9.5 Hz, 1 H) 7.36 (d, J = 9.5 Hz, 1 H) 9.44 (br s, 1 H) |
| 413 | 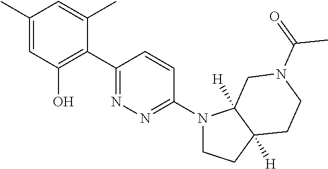 [SMILES: CC(=O)N1CC[C@H]2CCN(c3ccc(-c4c(C)cc(C)cc4O)nn3)[C@H]2C1] | 367 0.92 U | 1H NMR (400 MHz, DMSO-d6, 100° C.) d ppm 1.63-1.76 (m, 1 H), 1.84-1.98 (m, 1 H), 1.94 (br s, 3 H), 2.02-2.07 (m, 2 H), 2.08 (s, 3 H), 2.22 (s, 3 H), 2.51-2.58 (m partially hidden, 1 H), 3.26-3.37 (m, 1 H), 3.40-3.49 (m, 1 H), 3.55-3.66 (m, 2 H), 3.85-4.65 (m, 2 H), 4.13-4.29 (m, 1 H), 6.57 (br s, 1 H), 6.58 (br s, 1 H), 6.89 (br d, J = 9.5 Hz, 1 H), 7.31 (d, J = 9.5 Hz, 1 H), 9.07-9.25 (m, 1 H) |
| 414 | 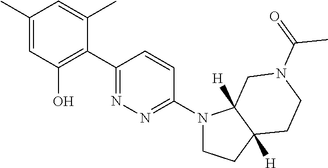 [SMILES: CC(=O)N1CC[C@@H]2CCN(c3ccc(-c4c(C)cc(C)cc4O)nn3)[C@@H]2C1] | 367 0.92 U | 1H NMR (400 MHz, DMSO-d6, 100° C.) d ppm 1.61-1.77 (m, 1 H), 1.85-1.98 (m, 1 H), 1.93 (br s, 3 H), 2.01-2.07 (m, 2 H), 2.08 (s, 3 H), 2.22 (s, 3 H), 2.51-2.59 (m partially hidden, 1 H), 3.26-3.37 (m, 1 H), 3.40-3.49 (m, 1 H), 3.56-3.66 (m, 2 H), 3.84-4.66 (m, 2 H), 4.14-4.24 (m, 1 H), 6.57 (br s, 1 H), 6.58 (br s, 1 H), 6.88 (br d, J = 9.5 Hz, 1 H), 7.31 (d, J = 9.5 Hz, 1 H), 9.10-9.24 (m, 1 H) |
| 415 | 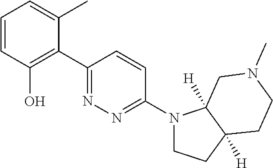 [SMILES: Cc1cccc(O)c1-c1ccc(N2CC[C@@H]3CCN(C)C[C@@H]32)nn1] | 325 0.60 and 0.63 U | 1H NMR (500 MHz, DMSO-d6, 27° C.) d ppm 1.66- 1.81 (m, 2 H), 1.85-2.05 (m, 3 H), 2.05-2.14 (m, 1 H), 2.06 (s, 3 H), 2.17 (s, 3 H), 2.33-2.41 (m, 1 H), 2.51-2.55 (m partially hidden, 1 H), 3.06 (br dd, J = 6.0 and 10.5 Hz, 1 H), 3.37-3.44 (m, 1 H), 3.52-3.60 (m, 1 H), 4.17-4.26 (m, 1 H), 6.71- 6.78 (m, 2 H), 6.88 (d, J = 9.5 Hz, 1 H), 7.08 (t, J = 8.0 Hz, 1 H), 7.27 (d, J = 9.5 Hz, 1 H), 9.46 (br s, 1 H) |
| 416 | 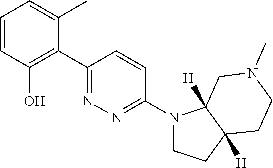 [SMILES: Cc1cccc(O)c1-c1ccc(N2CC[C@H]3CCN(C)C[C@H]32)nn1] | 325 0.61 and 0.63 U | 1H NMR (400 MHz, DMSO-d6, 30° C.) d ppm 1.67- 1.81 (m, 2 H), 1.87-2.05 (m, 3 H), 2.07 (s, 3 H), 2.05-2.14 (m, 1 H), 2.17 (s, 3 H), 2.32-2.41 (m, 1 H), 2.52-2.56 (m partially hidden, 1 H), 3.06 (br dd, J = 6.0 and 10.5 Hz, 1 H), 3.36-3.44 (m, 1 H), 3.52-3.60 (m, 1 H), 4.17-4.26 (m, 1 H), 6.71- 6.78 (m, 2 H), 6.88 (d, J = 9.5 Hz, 1 H), 7.08 (t, J = 8.0 Hz, 1 H), 7.27 (d, J = 9.5 Hz, 1 H), 9.45 (s, 1 H) |
| 417 | 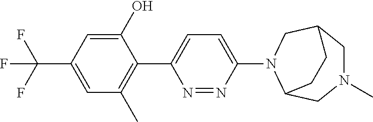 [SMILES: Cc1cc(C(F)(F)F)cc(O)c1-c1ccc(N2CC3CCC2CN(C)C3)nn1] | 393 0.91 B | 1H NMR (400 MHz, DMSO-d6, 100° C.) d ppm 1.58-1.69 (m, 1 H), 1.83-1.92 (m, 1 H), 1.96- 2.11 (m, 2 H), 2.18 (s, 3 H), 2.28 (s, 3 H), 2.29- 2.35 (m, 1 H), 2.35-2.43 (m, 2 H), 2.94-2.97 (m partially hidden, 2 H), 3.45 (br dd, J = 3.5 and 11.5 Hz, 1 H), 3.57 (br d, J = 11.5 Hz, 1 H), 4.79-4.87 (m, 1 H), 6.96 (d, J = 9.4 Hz, 1 H), 7.06 (br s, 2 H), 7.32 (d, J = 9.4 Hz, 1 H), 9.58-10.10 (m, 1 H) |
| 418 | 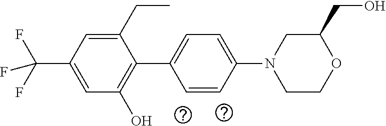 [SMILES: CCc1cc(C(F)(F)F)cc(O)c1-c1ccc(N2CCO[C@H](CO)C2)cc1] | 384.3 2.59 E | 1H NMR (500 MHz, DMSO) δ (ppm): 10.13 (s, 1H), 7.40 (d, J = 9.4 Hz, 1H), 7.31 (d, J = 9.4 Hz, 1H), 7.11 (s, 1H), 7.06 (s, 1H), 4.83 (t, J = 5.5 Hz, 1H), 4.35 (d, J = 12.7 Hz, 1H), 4.15 (d, J = 12.8 Hz, 1H), 4.04-3.96 (m, 1H), 3.62 (td, J = 11.6, 2.8 Hz, 1H), 3.57-3.50 (m, 2H), 3.47 (q, J = 6.3 Hz, 1H), 2.99 (td, J = 12.3, 3.5 Hz, 1H), 2.73 (dd, J = 12.9, 9.9 Hz, 1H), 2.45 (q, J = 7.7 Hz, 2H), 0.97 (t, J = 7.5 Hz, 3H). |
| 419 | 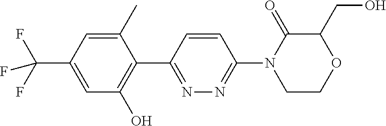 [SMILES: Cc1cc(C(F)(F)F)cc(O)c1-c1ccc(N2CCOC(CO)C2=O)nn1] | 384 1.23 B | 1H NMR (400 MHz, DMSO-d₆, 30° C.) δ ppm 2.12 (s, 3 H) 3.73-3.80 (m, 1 H) 3.87-3.93 (m, 1 H) 3.95-4.03 (m, 1 H) 4.06-4.15 (m, 1 H) 4.20-4.29 (m, 2 H) 4.37 (dd, J = 4.0, 2.5 Hz, 1 H) 5.01 (br t, J = 5.7 Hz, 1 H) 7.10 (br s, 1 H) 7.17 (br s, 1 H) 7.74 (d, J = 9.3 Hz, 1 H) 8.32 (d, J = 9.3 Hz, 1 H) 10.20- 10.47 (m, 1 H) |
| 420 | 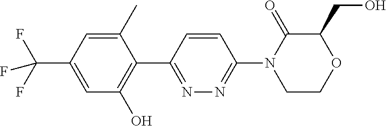 [SMILES: Cc1cc(C(F)(F)F)cc(O)c1-c1ccc(N2CCO[C@H](CO)C2=O)nn1] | 384 1.23 B | 1H NMR (400 MHz, DMSO-d₆, 30° C.) δ ppm 2.12 (s, 3 H) 3.73-3.80 (m, 1 H) 3.87-3.94 (m, 1 H) 3.95-4.03 (m, 1 H) 4.06-4.14 (m, 1 H) 4.20-4.29 (m, 2 H) 4.37 (dd, J = 4.0, 2.5 Hz, 1 H) 5.01 (br t, J = 5.7 Hz, 1 H) 7.11 (br s, 1 H) 7.17 (br s, 1 H) 7.74 (d, J = 9.3 Hz, 1 H) 8.32 (d, J = 9.3 Hz, 1 H) 10.19- 10.54 (m, 1 H) |
| 421 | 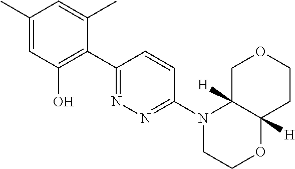 [SMILES: Cc1cc(C)c(-c2ccc(N3CCO[C@H]4CCOC[C@H]43)nn2)c(O)c1] | 342 1.10 U | 1H NMR (400 MHz, DMSO-d6, 30° C.) d ppm 1.63- 1.75 (m, 1 H), 1.83-1.90 (m, 1 H), 2.02 (s, 3 H), 2.23 (s, 3 H), 3.04 (dd, J = 9.7 and 10.6 Hz, 1 H), 3.20-3.27 (m partially hidden, 1 H), 3.35-3.40 (m partially hidden, 1 H), 3.47 (br td, J = 1.9 and 11.8 Hz, 1 H), 3.63-3.76 (m, 2 H), 3.84-3.99 (m, 3 H), 4.57 (dd, J = 3.7 and 10.6 Hz, 1 H), 6.59 (br s, 1 H), 6.60 (br s, 1 H), 7.36 (d, J = 9.3 Hz, 1 H), 7.44 (d, J = 9.3 Hz, 1 H), 9.34 - 9.43 (m, 1 H) |
| 422 | 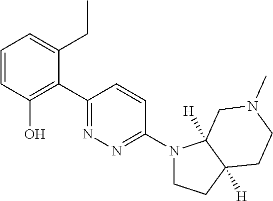 [SMILES: CCc1cccc(O)c1-c1ccc(N2CC[C@@H]3CCN(C)C[C@@H]32)nn1] | 339 0.74 U | 1H NMR (400 MHz, DMSO-d6, 30° C.) d ppm 0.95 (t, J = 7.5 Hz, 3 H), 1.67-1.81 (m, 2 H), 1.86-2.14 (m, 4 H), 2.17 (s, 3 H), 2.32-2.43 (m, 3 H), 2.52- 2.58 (m partially hidden, 1 H), 3.06 (br dd, J = 6.0 and 10.5 Hz, 1 H), 3.34-3.47 (m, 1 H), 3.51-3.60 (m, 1 H), 4.16-4.26 (m, 1 H), 6.75 (d, J = 7.8 Hz, 2 H), 6.87 (d, J = 9.3 Hz, 1 H), 7.12 (t, J = 7.8 Hz, 1 H), 7.23 (d, J = 9.3 Hz, 1 H), 9.30 (br s, 1 H) |
| 423 | 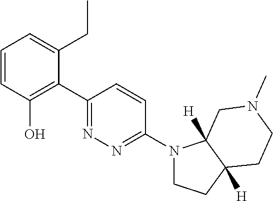 [SMILES: CCc1cccc(O)c1-c1ccc(N2CC[C@H]3CCN(C)C[C@H]32)nn1] | 339 0.72 and 0.73 U | 1H NMR (400 MHz, DMSO-d6, 30° C.) d ppm 0.95 (t, J = 7.5 Hz, 3 H), 1.67-1.81 (m, 2 H), 1.87-2.14 (m, 4 H), 2.17 (s, 3 H), 2.34-2.43 (m, 3 H), 2.52- 2.56 (m partially hidden, 1 H), 3.06 (br dd, J = 6.0 and 10.5 Hz, 1 H), 3.34-3.46 (m, 1 H), 3.51-3.61 (m, 1 H), 4.17-4.26 (m, 1 H), 6.75 (d, J = 7.8 Hz, 2 H), 6.87 (d, J = 9.3 Hz, 1 H), 7.12 (t, J = 7.8 Hz, 1 H), 7.23 (d, J = 9.3 Hz, 1 H), 9.30 (br s, 1 H) |
| 424 | 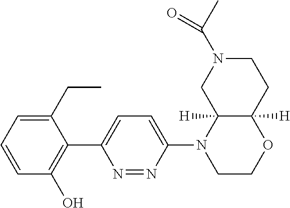 [SMILES: CCc1cccc(O)c1-c1ccc(N2CCO[C@@H]3CCN(C(C)=O)C[C@@H]32)nn1] | 383 1.04 U | 1H NMR (400 MHz, DMSO-d6, 100° C.) d ppm 0.98 (t, J = 7.5 Hz, 3 H), 1.80-1.88 (m, 2 H), 2.01 (s, 3 H), 2.42 (q, J = 7.5 Hz, 2 H), 2.68-3.38 (m partially hidden, 2 H), 3.25 (td, J = 3.8 and 12.7 Hz, 1 H), 3.57-4.47 (m partially hidden, 2 H), 3.71 (td, J = 3.3 and 11.8 Hz, 1 H), 3.89 (br q, J = 2.8 Hz, 1 H), 3.98 (br d, J = 12.7 Hz, 1 H), 4.09 (dd, J = 3.3 and 11.8 Hz, 1 H), 4.24-4.41 (m, 1 H), 6.77 (br d, J = 7.9 Hz, 2 H), 7.13 (t, J = 7.9 Hz, 1 H), 7.20 (br d, J = 9.3 Hz, 1 H), 7.34 (br d, J = 9.3 Hz, 1 H), 8.94 (br s, 1 H) |
| 425 | 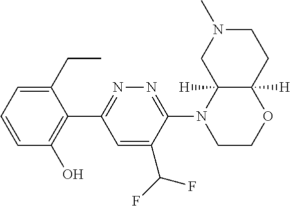 [SMILES: CCc1cccc(O)c1-c1cc(C(F)F)c(N2CCO[C@@H]3CCN(C)C[C@@H]32)nn1] | 405 0.96 U | 1H NMR (400 MHz, DMSO-d6, 30° C.) d ppm 0.95 (t, J = 7.5 Hz, 3 H), 1.74-1.86 (m, 2 H), 2.04-2.12 (m, 1 H), 2.16 (s, 3 H), 2.39 (q, J = 7.5 Hz, 2 H), 2.43-2.48 (m partially hidden, 2 H), 2.54-2.64 (m, 1 H), 3.30-3.33 (m partially hidden, 1 H), 3.61- 3.78 (m, 2 H), 3.82-3.89 (m, 1 H), 3.92 (br q, J = 2.8 Hz, 1 H), 3.96-4.02 (m, 1 H), 6.78-6.83 (m, 2 H), 7.21 (br t, J = 53.5 Hz, 1 H), 7.20 (t, J = 7.9 Hz, 1 H), 7.65 (s, 1 H), 9.50 (s, 1 H) |
| 426 | 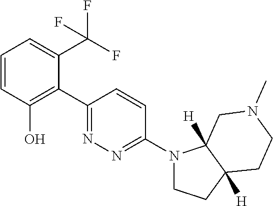 [SMILES: CN1CC[C@@H]2CCN(c3ccc(-c4c(O)cccc4C(F)(F)F)nn3)[C@@H]2C1] | 379 0.85 U | 1H NMR (400 MHz, DMSO-d6, 30° C.) d ppm 1.66- 1.81 (m, 2 H), 1.85-2.16 (m, 4 H), 2.17 (s, 3 H), 2.34-2.43 (m, 1 H), 2.53-2.57 (m partially hidden, 1 H), 3.06 (dd, J = 6.0 and 10.5 Hz, 1 H), 3.36-3.44 (m, 1 H), 3.52-3.60 (m, 1 H), 4.14- 4.27 (m, 1 H), 6.88 (d, J = 9.3 Hz, 1 H), 7.20-7.27 (m, 3 H), 7.43 (t, J = 8.0 Hz, 1 H), 9.90-10.19 (m, 1 H) |
| 427 | 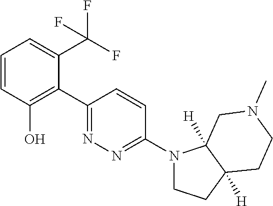 [SMILES: CN1CC[C@H]2CCN(c3ccc(-c4c(O)cccc4C(F)(F)F)nn3)[C@H]2C1] | 379 0.85 U | 1H NMR (400 MHz, DMSO-d6, 30° C.) d ppm 1.67- 1.81 (m, 2 H), 1.85-2.16 (m, 4 H), 2.17 (s, 3 H), 2.34-2.42 (m, 1 H), 2.53-2.57 (m partially hidden, 1 H), 3.06 (br dd, J = 6.0 and 10.5 Hz, 1 H), 3.36-3.45 (m, 1 H), 3.52-3.60 (m, 1 H), 4.14- 4.27 (m, 1 H), 6.88 (d, J = 9.3 Hz, 1 H), 7.20-7.27 (m, 3 H), 7.43 (t, J = 8.0 Hz, 1 H), 9.97-10.13 (m, 1 H) |
| 428 | 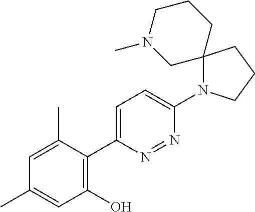 [SMILES: Cc1cc(C)c(-c2ccc(N3CCCC34CCCN(C)C4)nn2)c(O)c1] | 353 0.80 B | 1H NMR (500 MHz, DMSO-d₆, 27° C.) δ ppm 1.44- 1.81 (m, 8 H) 2.03 (s, 3 H) 2.22 (s, 3 H) 2.35 (br s, 3 H) 2.72-2.87 (m, 4 H) 4.02 (br d, J = 12.6 Hz, 1 H) 4.36 (br d, J = 12.6 Hz, 1 H) 6.56 (br s, 1 H) 6.57 (br s, 1 H) 7.25-7.26 (m, 2 H) 9.41 (br s, 1 H) |
| 429 | 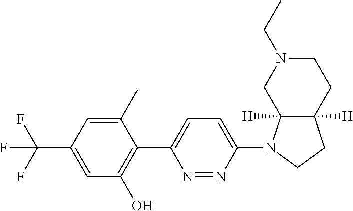 [SMILES: CCN1CC[C@H]2CCN(c3ccc(-c4c(C)cc(C(F)(F)F)cc4O)nn3)[C@H]2C1] | 407.5 2.73 E | ¹H NMR (500 MHz, DMSO-d₆) δ (ppm): 10.14 (s, 1H), 7.31 (d, J = 9.3 Hz, 1H), 7.11 (s, 1H), 7.06 (s, 1H), 6.92 (d, J = 9.4 Hz, 1H), 4.31-4.13 (m, 1H), 3.68-3.51 (m, 1H), 3.49-3.37 (m, 1H), 3.20- 3.08 (m, 1H), 2.68-2.59 (m, 1H), 2.45-2.29 (m, 3H), 2.20-2.02 (m, 5H), 2.01-1.87 (m, 2H), 1.81 (t, J = 10.6 Hz, 1H), 1.73-1.67 (m, 1H), 1.00 (t, J = 7.1 Hz, 3H). |
| 430 | 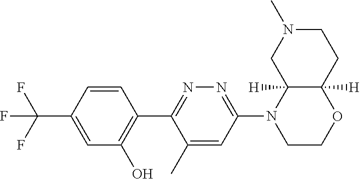 [SMILES: Cc1cc(N2CCO[C@@H]3CCN(C)C[C@@H]32)nnc1-c1ccc(C(F)(F)F)cc1O] | 409 0.93 U | 1H NMR (400 MHz, DMSO-d6, 30° C.) d ppm 1.75- 1.94 (m, 2 H), 2.07-2.15 (m, 1 H), 2.10 (s, 3 H), 2.19 (s, 3 H), 2.30-2.38 (m, 1 H), 2.52-2.58 (m partially hidden, 2 H), 3.15 (td, J = 3.8 and 12.7 Hz, 1 H), 3.61 (td, J = 3.3 and 11.8 Hz, 1 H), 3.73 (br q, J = 2.8 Hz, 1 H), 3.93 (br d, J = 12.7 Hz, 1 H), 4.05 (dd, J = 3.3 and 11.8 Hz, 1 H), 4.37-4.49 (m, 1 H), 7.12 (br s, 1 H), 7.22 (br s, 1 H), 7.24 (br d, J = 7.8 Hz, 1 H), 7.41 (d, J = 7.8 Hz, 1 H), 10.44 (s, 1 H) |
| 431 | 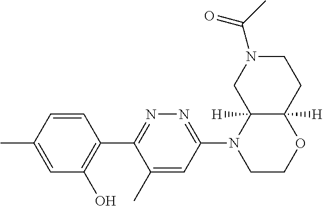 [SMILES: CC(=O)N1CC[C@H]2OCCN(c3cc(C)c(-c4ccc(C)cc4O)nn3)[C@H]2C1] | 383 0.95 U | 1H NMR (400 MHz, DMSO-d6, 100° C.) d ppm 1.80-1.92 (m, 2 H), 2.04 (s, 3 H), 2.17 (br s, 3 H), 2.31 (s, 3 H), 2.73-3.38 (m partially hidden, 2 H), 3.25 (td, J = 3.8 and 12.7 Hz, 1 H), 3.54-4.26 (m partially hidden, 2 H), 3.71 (td, J = 3.3 and 11.8 Hz, 1 H), 3.88 (br q, J = 2.8 Hz, 1 H), 3.97 (br d, J = 12.7 Hz, 1 H), 4.10 (dd, J = 3.3 and 11.8 Hz, 1 H), 4.28- 4.40 (m, 1 H), 6.73 (br d, J = 7.8 Hz, 1 H), 6.77 (br s, 1 H), 7.08 (br s, 1 H), 7.12 (d, J = 7.8 Hz, 1 H), 9.39- 9.53 (m, 1 H) |
| 432 | 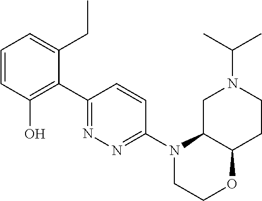 [SMILES: CCc1cccc(O)c1-c1ccc(N2CCO[C@@H]3CCN(C(C)C)C[C@@H]32)nn1] | 383 0.87 U | 1H NMR (400 MHz, DMSO-d6, 30° C.) d ppm 0.92- 1.07 (m, 9 H), 1.81-1.93 (m, 2 H), 2.26-3.00 (m partially hidden, 5 H), 2.38 (q, J = 7.3 Hz, 2 H), 3.20 (br td, J = 3.8 and 12.7 Hz, 1 H), 3.64 (br td, J = 3.3 and 11.8 Hz, 1 H), 3.75-3.81 (m, 1 H), 3.94 (br d, J = 12.7 Hz, 1 H), 4.06 (dd, J = 3.3 and 11.8 Hz, 1 H), 4.25-4.63 (m, 1 H), 6.77 (d, J = 7.9 Hz, 2 H), 7.14 (t, J = 7.9 Hz, 1 H), 7.20 (d, J = 9.5 Hz, 1 H), 7.35 (d, J = 9.5 Hz, 1 H), 9.36 (s, 1 H) |
| 433 | 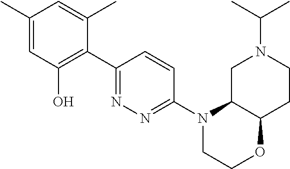 [SMILES: Cc1cc(C)c(-c2ccc(N3CCO[C@@H]4CCN(C(C)C)C[C@@H]43)nn2)c(O)c1] | 383 0.87 U | 1H NMR (400 MHz, DMSO-d6, 30° C.) d ppm 0.96 (br dd, J = 6.8 and 10.1 Hz, 6 H), 1.77-1.86 (m, 2 H), 2.03 (s, 3 H), 2.22 (s, 3 H), 2.28-2.40 (m, 1 H), 2.52-2.58 (m partially hidden, 2 H), 2.58-2.65 (m, 1 H), 2.69-2.80 (m, 1 H), 3.18 (td, J = 3.8 and 12.7 Hz, 1 H), 3.62 (td, J = 3.3 and 11.8 Hz, 1 H), 3.76 (br q, J = 2.8 Hz, 1 H), 3.93 (br d, J = 12.7 Hz, 1 H), 4.05 (dd, J = 3.3 and 11.8 Hz, 1 H), 4.24-4.46 (m, 1 H), 6.57 (br s, 1 H), 6.58 (br s, 1 H), 7.18 (d, J = 9.5 Hz, 1 H), 7.34 (d, J = 9.5 Hz, 1 H), 9.38 (s, 1 H) |
| 434 | 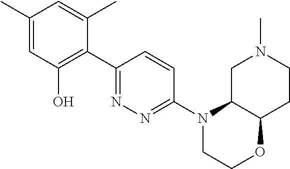 [SMILES: Cc1cc(C)c(-c2ccc(N3CCO[C@@H]4CCN(C)C[C@@H]43)nn2)c(O)c1] | 369 0.85 U | 1H NMR (400 MHz, DMSO-d6, 30°° C.) d ppm 1.70- 1.87 (m, 2 H), 2.01 (s, 3 H), 2.03-2.12 (m, 1 H), 2.15 (s, 3 H), 2.23 (s, 3 H), 2.28 (s, 3 H), 2.38-2.48 (m partially hidden, 2 H), 2.59 (t, J = 10.9 Hz, 1 H), 3.24-3.29 (m partially hidden, 1 H), 3.56-3.69 (m, 2 H), 3.74 (td, J = 2.6 and 11.6 Hz, 1 H), 3.85 (q, J = 2.8 Hz, 1 H), 3.97 (br dd, J = 2.6 and 11.0 Hz, 1 H), 6.58 (br s, 1 H), 6.59 (br s, 1 H), 7.30 (br s, 1 H), 9.38 (s, 1 H) |
| 435 | 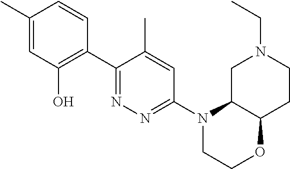 [SMILES: CCN1CC[C@H]2OCCN(c3cc(C)c(-c4ccc(C)cc4O)nn3)[C@H]2C1] | 369 1.71 Z2 | 1H NMR (400 MHz, DMSO-d₆, 30° C.) δ ppm 0.98 (t, J = 7.3 Hz, 3 H) 1.75-1.94 (m, 2 H) 2.03-2.16 (m, 1 H) 2.10 (s, 3 H) 2.28 (s, 3 H) 2.30-2.44 (m, 3 H) 2.56-2.65 (m, 2 H) 3.14 (td, J = 12.7, 3.8 Hz, 1 H) 3.60 (td, J=11.8, 3.3 Hz, 1 H) 3.73 (br q, J = 2.8 Hz, 1 H) 3.90 (br d, J = 12.7 Hz, 1 H) 4.04 (dd, J = 11.8, 3.3 Hz, 1 H) 4.33-4.47 (m, 1 H) 6.70 (br d, J = 7.8 Hz, 1 H) 6.73 (br s, 1 H) 7.05-7.08 (m, 2 H) 9.65 (s, 1 H) |
| 436 | 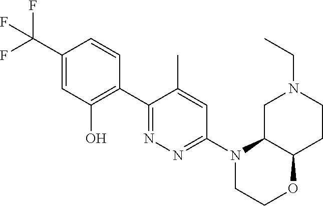 [SMILES: CCN1CC[C@H]2OCCN(c3cc(C)c(-c4ccc(C(F)(F)F)cc4O)nn3)[C@H]2C1] | 423 2.54 Z2 | 1H NMR (400 MHz, DMSO-d₆, 30° C.) δ ppm 0.98 (t, J = 7.3 Hz, 3 H) 1.75-1.92 (m, 2 H) 2.04-2.13 (m, 1 H) 2.10 (br s, 3 H) 2.26-2.45 (m, 3 H) 2.56- 2.65 (m, 2 H) 3.16 (td, J = 12.7, 3.8 Hz, 1 H) 3.61 (td, J = 11.8, 3.3 Hz, 1 H) 3.74 (q, J = 2.8 Hz, 1 H) 3.93 (br d, J = 12.7 Hz, 1 H) 4.05 (dd, J = 11.8, 3.3 Hz, 1 H) 4.38-4.47 (m, 1 H) 7.12 (br s, 1 H) 7.22 (d, J = 1.6 Hz, 1 H) 7.24 (dd, J = 7.8, 1.6 Hz, 1 H) 7.41 (d, J = 7.8 Hz, 1 H) 10.38-10.51 (m, 1 H) |
| 437 | 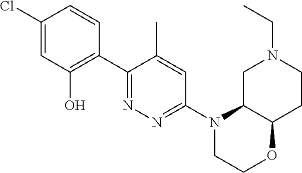 [SMILES: CCN1CC[C@H]2OCCN(c3cc(C)c(-c4ccc(Cl)cc4O)nn3)[C@H]2C1] | 389 2.14 Z2 | 1H NMR (400 MHz, DMSO-d6, 30° C.) d ppm 0.97 (br t, J = 7.3 Hz, 3 H), 1.76-1.92 (m, 2 H), 2.03- 2.15 (m, 1 H), 2.09 (br s, 3 H), 2.31-2.42 (m, 3 H), 2.57-2.67 (m partially hidden, 2 H), 3.15 (br t, J = 12.7 Hz, 1 H), 3.60 (br t, J = 11.8 Hz, 1 H), 3.69- 3.76 (m, 1 H), 3.91 (br d, J = 12.7 Hz, 1 H), 4.04 (br d, J = 11.8 Hz, 1 H), 4.34-4.46 (m, 1 H), 6.90-7.01 (m, 2 H), 7.09 (br s, 1 H), 7.20 (br d, J = 7.8 Hz, 1 H), 10.25 (br s, 1 H) |
| 438 | 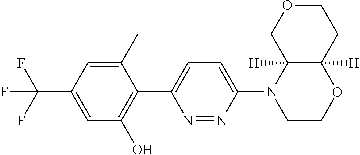 [SMILES: Cc1cc(C(F)(F)F)cc(O)c1-c1ccc(N2CCO[C@@H]3CCOC[C@@H]32)nn1] | 396 1.41 U | 1H NMR (400 MHz, DMSO-d6, 30° C.) d ppm 1.75- 1.82 (m, 1 H), 1.92-2.04 (m, 1 H), 2.13 (s, 3 H), 3.18 (td, J = 3.8 and 12.7 Hz, 1 H), 3.44-3.78 (m, 5 H), 3.91 (br q, J = 2.8 Hz, 1 H), 3.93-3.99 (m, 1 H), 4.09 (dd, J = 3.3 and 11.8 Hz, 1 H), 4.42-4.54 (m, 1 H), 7.06 (br s, 1 H), 7.11 (br s, 1 H), 7.29 (d, J = 9.5 Hz, 1 H), 7.40 (d, J = 9.5 Hz, 1 H), 10.06-10.23 (m, 1 H) |
| 439 | 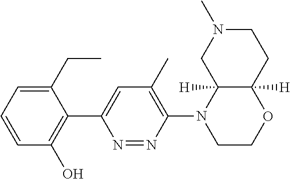 [SMILES: CCc1cccc(O)c1-c1cc(C)c(N2CCO[C@@H]3CCN(C)C[C@@H]32)nn1] | 369 0.89 U | 1H NMR (400 MHz, DMSO-d6, 30° C.) d ppm 0.94 (t, J = 7.6 Hz, 3 H), 1.71-1.88 (m, 2 H), 2.04-2.13 (m, 1 H), 2.15 (s, 3 H), 2.28 (br s, 3 H), 2.35 (q, J = 7.6 Hz, 2 H), 2.38-2.48 (m partially hidden, 2 H), 2.60 (br t, J = 11.0 Hz, 1 H), 3.25-3.31 (m partially hidden, 1 H), 3.57-3.69 (m, 2 H), 3.74 (td, J = 2.5 and 11.8 Hz, 1 H), 3.85 (br q, J = 2.8 Hz, 1 H), 3.97 (dd, J = 2.5 and 11.0 Hz, 1 H), 6.78 (d, J = 7.8 Hz, 2 H), 7.16 (t, J = 7.8 Hz, 1 H), 7.31 (br s, 1 H), 9.33 (s, 1 H) |
| 440 | 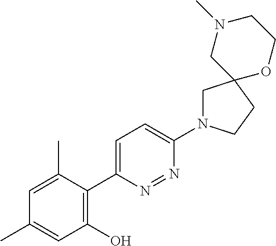 [SMILES: Cc1cc(C)c(-c2ccc(N3CCC4(CN(C)CCO4)C3)nn2)c(O)c1] | 355 0.69 and 0.7 U | 1H NMR (400 MHz, DMSO-d6, 30° C.) d ppm 1.98- 2.07 (m, 1 H), 2.03 (s, 3 H), 2.11-2.18 (m, 1 H), 2.19 (s, 3 H), 2.22 (s, 3 H), 2.24-2.29 (m, 1 H), 2.29-2.33 (m, 1 H), 2.34-2.41 (m, 1 H), 2.47- 2.53 (m hidden, 1 H), 3.43-3.63 (m, 3 H), 3.64- 3.74 (m, 3 H), 6.56 (br s, 1 H), 6.57 (br s, 1 H), 6.89 (d, J = 9.3 Hz, 1 H), 7.27 (d, J = 9.3 Hz, 1 H), 9.38 (s, 1 H) |
| 441 | 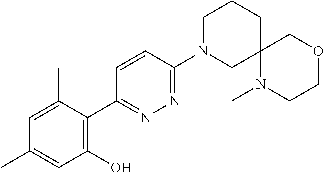 [SMILES: Cc1cc(C)c(-c2ccc(N3CCCC4(COCCN4C)C3)nn2)c(O)c1] | 369 0.79 U | 1H NMR (400 MHz, DMSO-d6, 30° C.) d ppm 1.55- 1.76 (m, 3 H), 1.78-1.89 (m, 1 H), 2.03 (s, 3 H), 2.22 (s, 3 H), 2.33 (s, 3 H), 2.53-2.56 (m partially hidden, 1 H), 2.69-2.78 (m, 1 H), 2.84-2.93 (m, 1 H), 3.21 (d, J = 12.9 Hz, 1 H), 3.28-3.30 (m partially hidden, 1 H), 3.53 (d, J = 11.3 Hz, 1 H), 3.56-3.61 (m, 1 H), 3.61-3.69 (m, 1 H), 4.22 (br d, J = 12.9 Hz, 1 H), 4.24-4.31 (m, 1 H), 6.56 (br s, 1 H), 6.57 (br s, 1 H), 7.20 (d, J = 9.5 Hz, 1 H), 7.28 (d, J = 9.5 Hz, 1 H), 9.36-9.47 (m, 1 H) |
| 442 | 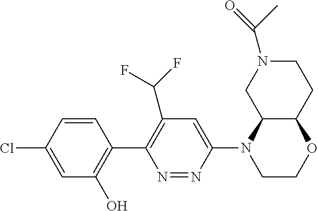 [SMILES: CC(=O)N1CC[C@H]2OCCN(c3cc(C(F)F)c(-c4ccc(Cl)cc4O)nn3)[C@H]2C1] | 439 1.41 U | 1H NMR (400 MHz, DMSO-d6, 100° C.) d ppm 1.83-1.92 (m, 2 H), 2.04 (s, 3 H), 2.73-3.24 (m partially hidden, 2 H), 3.32 (td, J = 3.8 and 12.7 Hz, 1 H), 3.53-4.31 (m partially hidden, 2 H), 3.73 (td, J = 3.3 and 11.8 Hz, 1 H), 3.91 (br q, J = 2.8 Hz, 1 H), 4.05-4.15 (m, 2 H), 4.36-4.51 (m, 1 H), 6.76 (t, J = 54.5 Hz, 1 H), 6.98 (dd, J = 2.0 and 8.1 Hz, 1 H), 7.01 (d, J = 2.0 Hz, 1 H), 7.30 (d, J = 8.1 Hz, 1 H), 7.36 (br s, 1 H), 9.45-10.55 (m, 1 H) |
| 443 | 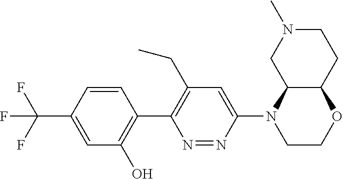 [SMILES: CCc1cc(N2CCO[C@@H]3CCN(C)C[C@@H]32)nnc1-c1ccc(C(F)(F)F)cc1O] | 423 1.04 U | 1H NMR (400 MHz, DMSO-d6, 30° C.) d ppm 1.00 (t, J = 7.6 Hz, 3 H), 1.76-1.93 (m, 2 H), 2.04-2.13 (m, 1 H), 2.18 (s, 3 H), 2.29-2.36 (m, 1 H), 2.40 (q, J = 7.6 Hz, 2 H), 2.52-2.57 (m partially hidden, 2 H), 3.16 (td, J = 3.8 and 12.7 Hz, 1 H), 3.62 (td, J = 3.3 and 11.8 Hz, 1 H), 3.73 (br q, J = 2.8 Hz, 1 H), 3.94 (br d, J = 12.7 Hz, 1 H), 4.05 (dd, J = 3.3 and 11.8 Hz, 1 H), 4.42-4.51 (m, 1 H), 7.06 (s, 1 H), 7.21 (br s, 1 H), 7.24 (br d, J = 7.8 Hz, 1 H), 7.39 (d, J = 7.8 Hz, 1 H), 10.25-10.46 (m, 1 H) |
| 444 | 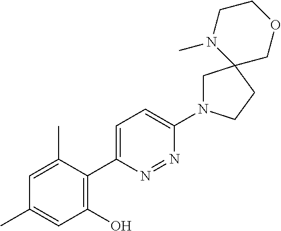 [SMILES: Cc1cc(C)c(-c2ccc(N3CCC4(COCCN4C)C3)nn2)c(O)c1] | 355 0.66 K | 1H NMR (400 MHz, DMSO-d6, 30° C.) d ppm 1.79- 1.87 (m, 1 H), 2.04 (s, 3 H), 2.20-2.26 (m, 1 H), 2.22 (s, 3 H), 2.24 (s, 3 H), 2.52-2.53 (m hidden, 2 H), 3.32-3.36 (m partially hidden, 1 H), 3.42-3.55 (m, 3 H), 3.59-3.69 (m, 4 H), 6.57 (br s, 1 H), 6.57 (br s, 1 H), 6.93 (d, J = 9.3 Hz, 1 H), 7.29 (d, J = 9.3 Hz, 1 H), 9.37 (s, 1 H) |
| 445 | 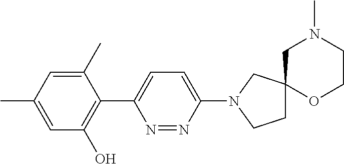 [SMILES: Cc1cc(C)c(-c2ccc(N3CC[C@]4(CN(C)CCO4)C3)nn2)c(O)c1] | 355 0.67 K | 1H NMR (400 MHz, DMSO-d6, 30° C.) d ppm 1.98- 2.06 (m, 1 H), 2.03 (s, 3 H), 2.12-2.17 (m, 1 H), 2.19 (s, 3 H), 2.22 (s, 3 H), 2.23-2.29 (m, 1 H), 2.29-2.34 (m, 1 H), 2.34-2.40 (m, 1 H), 2.46- 2.50 (m partially hidden, 1 H), 3.47-3.63 (m, 3 H), 3.63-3.73 (m, 3 H), 6.56 (br s, 1 H), 6.57 (br s, 1 H), 6.89 (d, J = 9.3 Hz, 1 H), 7.27 (d, J = 9.3 Hz, 1 H), 9.39 (br s, 1 H) |
| 446 | 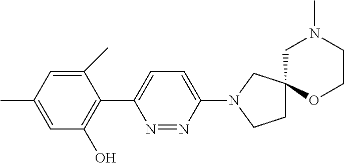 [SMILES: Cc1cc(C)c(-c2ccc(N3CC[C@@]4(CN(C)CCO4)C3)nn2)c(O)c1] | 355 0.66 K | 1H NMR (400 MHz, DMSO-d6, 30° C.) d ppm 1.98- 2.06 (m, 1 H), 2.03 (s, 3 H), 2.13-2.17 (m, 1 H), 2.19 (s, 3 H), 2.22 (s, 3 H), 2.24-2.29 (m, 1 H), 2.29-2.34 (m, 1 H), 2.34-2.41 (m, 1 H), 2.46- 2.49 (m partially hidden, 1 H), 3.48-3.63 (m, 3 H), 3.63-3.73 (m, 3 H), 6.56 (br s, 1 H), 6.57 (br s, 1 H), 6.89 (d, J = 9.3 Hz, 1 H), 7.27 (d, J = 9.3 Hz, 1 H), 9.39 (br s, 1 H) |
| 448 | 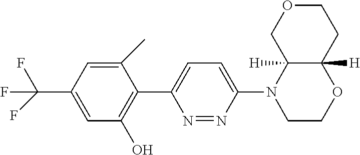 [SMILES: Cc1cc(C(F)(F)F)cc(O)c1-c1ccc(N2CCO[C@H]3CCOC[C@@H]32)nn1] | 396 1.46 U | 1H NMR (400 MHz, DMSO-d6, 30° C.) d ppm 1.64- 1.75 (m, 1 H), 1.85-1.91 (m, 1 H), 2.12 (s, 3 H), 3.07 (dd, J = 9.7 and 10.6 Hz, 1 H), 3.33-3.44 (m partially hidden, 2 H), 3.48 (td, J = 1.9 and 11.8 Hz, 1 H), 3.70-3.78 (m, 2 H), 3.85-4.00 (m, 3 H), 4.59 (dd, J = 3.7 and 10.6 Hz, 1 H), 7.08 (br s, 1 H), 7.13 (br s, 1 H), 7.42 (d, J = 9.3 Hz, 1 H), 7.51 (d, J = 9.3 Hz, 1 H), 10.09-10.29 (m, 1 H) |
| 449 | 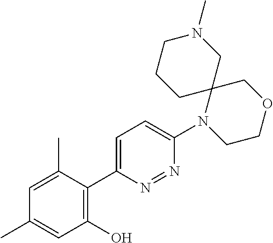 [SMILES: Cc1cc(C)c(-c2ccc(N3CCOCC34CCCN(C)C4)nn2)c(O)c1] | 369 0.79 U | 1H NMR (400 MHz, DMSO-d6, 30°° C.) d ppm 1.54- 1.74 (m, 3 H), 1.77-1.89 (m, 1 H), 2.03 (s, 3 H), 2.22 (s, 3 H), 2.33 (br s, 3 H), 2.54-2.56 (m, 1 H), 2.69-2.80 (m, 1 H), 2.84-2.94 (m, 1 H), 3.21 (br d, J = 12.0 Hz, 1 H), 3.27-3.30 (m hidden, 1 H), 3.54 (br d, J = 11.8 Hz, 1 H), 3.56-3.69 (m, 2 H), 4.22 (d, J = 12.0 Hz, 1 H), 4.24 .31 (m, 1 H), 6.56 (br s, 1 H), 6.57 (br s, 1 H), 7.21 (d, J = 9.5 Hz, 1 H), 7.28 (d, J = 9.5 Hz, 1 H), 9.41 (br s, 1 H) |
| 450 | 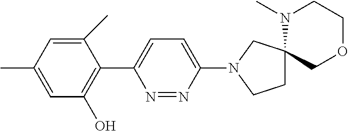 [SMILES: Cc1cc(C)c(-c2ccc(N3CC[C@]4(COCCN4C)C3)nn2)c(O)c1] | 355 0.74 U | 1H NMR (400 MHz, DMSO-d6, 30° C.) d ppm 1.76- 1.88 (m, 1 H), 2.03 (s, 3 H), 2.19-2.26 (m, 1 H), 2.22 (s, 3 H), 2.24 (s, 3 H), 2.52-2.53 (m partially hidden, 2 H), 3.33-3.36 (m partially hidden, 1 H), 3.44-3.54 (m, 3 H), 3.61-3.67 (m, 4 H), 6.57 (br s, 1 H), 6.57 (s, 1 H), 6.93 (d, J = 9.3 Hz, 1 H), 7.29 (d, J = 9.3 Hz, 1 H), 9.39 (br s, 1 H) |
| 451 | 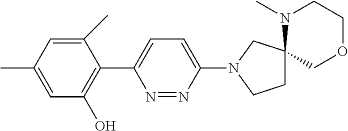 [SMILES: Cc1cc(C)c(-c2ccc(N3CC[C@@]4(COCCN4C)C3)nn2)c(O)c1] | 355 0.73 U | 1H NMR (400 MHz, DMSO-d6, 30° C.) d ppm 1.77 1.89 (m, 1 H), 2.03 (s, 3 H), 2.17-2.26 (m, 1 H), 2.22 (s, 3 H), 2.24 (s, 3 H), 2.51-2.53 (m partially hidden, 2 H), 3.31-3.36 (m partially hidden, 1 H), 3.42-3.55 (m, 3 H), 3.60-3.68 (m, 4 H), 6.56 (br s, 1 H), 6.57 (br s, 1 H), 6.93 (d, J = 9.3 Hz, 1 H), 7.29 (d, J = 9.3 Hz, 1 H), 9.38 (br s, 1 H) |
| 452 | 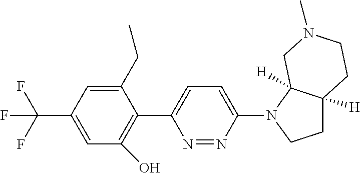 [SMILES: CCc1cc(C(F)(F)F)cc(O)c1-c1ccc(N2CC[C@@H]3CCN(C)C[C@@H]32)nn1] | 407.4 2.81 E | ¹H NMR (400 MHz, DMSO) δ (ppm): 10.13 (s, 1H), 7.29 (d, J = 9.3 Hz, 1H), 7.07 (s, 1H), 7.03 (s, 1H), 6.91 (d, J = 9.4 Hz, 1H), 4.21 (s, 1H), 3.60- 3.55 (m, 1H), 3.45-3.38 (m, 1H), 3.06 (dd, J = 11.3, 5.8 Hz, 1H), 2.58-2.53 (m, 1H), 2.47-2.34 (m, 3H), 2.17 (s, 3H), 2.14-1.86 (m, 4H), 1.80- 1.66 (m, 2H), 0.97 (t, J = 7.5 Hz, 3H). |
| 455 | 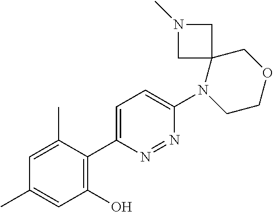 [SMILES: Cc1cc(C)c(-c2ccc(N3CCOCC34CN(C)C4)nn2)c(O)c1] | 341 0.84 U | 1H NMR (400 MHz, DMSO-d6, 30° C.) d ppm 2.01 (s, 3 H), 2.22 (s, 3 H), 2.38 (s, 3 H), 2.45-2.48 (m, 2 H), 3.61-3.65 (m, 2 H), 3.69 (br s, 2 H), 3.70 (d, J = 9.1 Hz, 2 H), 4.20 (d, J = 9.1 Hz, 2 H), 6.56 (br s, 1 H), 6.57 (br s, 1 H), 6.84 (d, J = 9.1 Hz, 1 H), 7.30 (d, J = 9.1 Hz, 1 H), 9.35 (s, 1 H) |
| 456 | 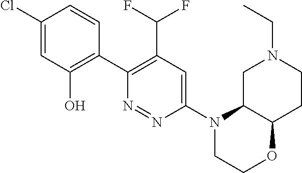 [SMILES: CCN1CC[C@H]2OCCN(c3cc(C(F)F)c(-c4ccc(Cl)cc4O)nn3)[C@H]2C1] | 373 0.78 U | 1H NMR (400 MHz, DMSO-d6, 30° C.) d ppm 0.97 (t, J = 7.3 Hz, 3 H), 1.77-1.92 (m, 2 H), 2.04-2.14 (m, 1 H), 2.33-2.43 (m, 3 H), 2.57-2.66 (m, 2 H), 3.22 (td, J = 3.8 and 12.7 Hz, 1 H), 3.63 (td, J = 3.3 and 11.8 Hz, 1 H), 3.75 (br q, J = 2.8 Hz, 1 H), 3.96- 4.09 (m, 2 H), 4.46-4.59 (m, 1 H), 6.80 (t, J = 54.3 Hz, 1 H), 6.96-7.01 (m, 2 H), 7.30 (br d, J = 7.8 Hz, 1 H), 7.32 (br s, 1 H), 10.06-10.79 (m, 1 H) |
| 457 | 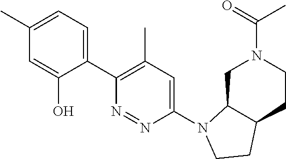 [SMILES: CC(=O)N1CC[C@H]2CCN(c3cc(C)c(-c4ccc(C)cc4O)nn3)[C@H]2C1] | 367.5 0.93 U | 1H NMR (400 MHz, DMSO-d6, 100° C.) d ppm 1.62-1.76 (m, 1 H), 1.83-1.97 (m, 1 H), 1.94 (br s, 3 H), 2.00-2.09 (m, 2 H), 2.14 (s, 3 H), 2.28 (s, 3 H), 2.50-2.58 (m partially hidden, 1 H), 3.24-3.34 (m, 1 H), 3.37-3.48 (m, 1 H), 3.56-3.66 (m, 2 H), 3.80-4.67 (m, 2 H), 4.14-4.26 (m, 1 H), 6.70 (br d, J = 7.8 Hz, 1 H), 6.72 (br s, 1 H), 6.74 (br s, 1 H), 7.09 (d, J = 7.8 Hz, 1 H), 9.46-9.59 (m, 1 H) |
| 458 | 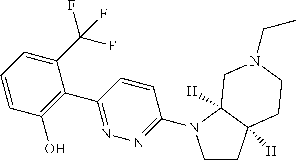 [SMILES: CCN1CC[C@H]2CCN(c3ccc(-c4c(O)cccc4C(F)(F)F)nn3)[C@H]2C1] | 393.4 2.4 E | ¹H NMR (400 MHz, DMSO) δ 10.08 (s, 1H), 7.44 (t, J = 8.0 Hz, 1H), 7.37-7.16 (m, 3H), 6.88 (d, J = 9.2 Hz, 1H), 4.22 (s, 1H), 3.56 (m, 1H), 3.40 (m, 1H), 3.16 (m, 1H), 2.80-2.60 (m, 1H), 2.36 (m, 3H), 2.27-2.02 (m, 2H), 1.92 (m, 2H), 1.81 (m, 1H), 1.72 (m, 1H), 1.00 (t, J = 7.1 Hz, 3H). |
| 459 | 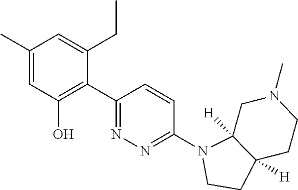 [SMILES: CCc1cc(C)cc(O)c1-c1ccc(N2CC[C@@H]3CCN(C)C[C@@H]32)nn1] | 353.4 2.52 E | 1H NMR (400 MHz, DMSO-d₆) δ (ppm): 9.23 (s, 1H), 7.21 (d, J = 9.2 Hz, 1H), 6.87 (d, J = 9.3 Hz, 1H), 6.57 (d, J = 3.2 Hz, 2H), 4.20 (s, 1H), 3.56 (t, J = 9.5 Hz, 1H), 3.49-3.36 (m, 1H), 3.06 (dd, J = 11.2, 5.8 Hz, 1H), 2.54 (s, 1H), 2.36 (qd, J = 6.8, 2.9 Hz, 3H), 2.24 (s, 3H), 2.17 (s, 3H), 2.14-1.84 (m, 4H), 1.81-1.66 (m, 2H), 0.94 (t, J = 7.5 Hz, 3H) |
| 460 | 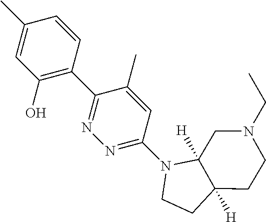 [SMILES: CCN1CC[C@H]2CCN(c3cc(C)c(-c4ccc(C)cc4O)nn3)[C@H]2C1] | 353.3 2.43 E | ¹H NMR (400 MHz, DMSO-d₆) δ (ppm): 9.63 (s, 1H), 6.98 (d, J = 7.6 Hz, 1H), 6.68-6.59 (m, 3H), 4.13 (s, 1H), 3.46 (t, J = 9.5 Hz, 1H), 3.34 (s, 1H), 3.11-3.03 (m, 1H), 2.59-2.53 (m, 1H), 2.32- 2.24 (m, 3H), 2.21 (s, 3H), 2.01 (s, 3H), 2.08-1.91 (m, 2H), 1.91-1.77 (m, 2H), 1.74-1.60 (m, 2H), 0.92 (t, J = 7.1 Hz, 3H). |
| 461 | 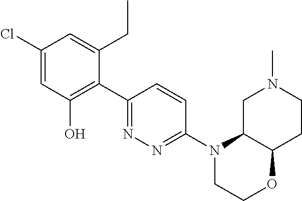 [SMILES: CCc1cc(Cl)cc(O)c1-c1ccc(N2CCO[C@@H]3CCN(C)C[C@@H]32)nn1] | 389 0.99 U | 1H NMR (400 MHz, DMSO-d6, 30° C.) d ppm 0.95 (t, J = 7.5 Hz, 3 H), 1.76-1.94 (m, 2 H), 2.05-2.15 (m, 1 H), 2.19 (s, 3 H), 2.33-2.41 (m, 3 H), 2.51- 2.56 (m partially hidden, 2 H), 3.16 (td, J = 3.8 and 12.7 Hz, 1 H), 3.63 (td, J = 3.3 and 11.8 Hz, 1 H), 3.74 (br q, J = 2.8 Hz, 1 H), 3.93 (br d, J = 12.7 Hz, 1 H), 4.05 (dd, J = 3.3 and 11.8 Hz, 1 H), 4.38-4.45 (m, 1 H), 6.81 (d, J = 2.0 Hz, 1 H), 6.84 (d, J = 2.0 Hz, 1 H), 7.21 (d, J = 9.5 Hz, 1 H), 7.34 (d, J = 9.5 Hz, 1 H), 9.87 (br s, 1 H) |
| 462 | 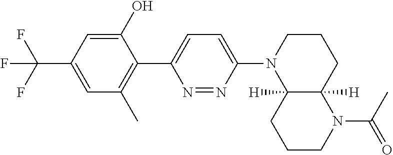 [SMILES: CC(=O)N1CCC[C@@H]2[C@H]1CCCN2c1ccc(-c2c(C)cc(C(F)(F)F)cc2O)nn1] | 435 1.34 U | 1H NMR (400 MHz, DMSO-d6, 30° C., mixture rotamers + atropoisomers) d ppm 1.35-1.67 (m, 4 H), 1.69-1.80 (m, 1 H), 1.80-1.92 (m, 1 H), 1.92- 2.24 (m, 2 H), 2.03 (s, 1.7 H), 2.06 (s, 1.3 H), 2.13 (s, 3 H), 2.54-2.64 (m, 0.45 H), 2.88-3.02 (m, 1 H), 3.05-3.16 (m, 0.55 H), 3.60-3.70 (m, 0.55 H), 3.93-4.03 (m, 0.45 H), 4.24 (br d, J = 12.7 Hz, 0.55 H), 4.33 (br d, J = 12.7 Hz, 0.9 H), 4.44-4.53 (m, 0.55 H), 4.54-4.67 (m, 1 H), 7.06 (s, 1 H), 7.11 (s, 1 H), 7.30-7.43 (m, 2 H), 10.15 (br s, 1 H) |
| 463 | 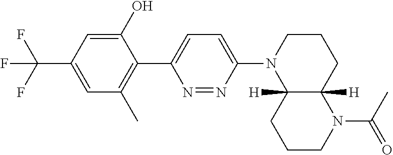 [SMILES: CC(=O)N1CCC[C@H]2[C@@H]1CCCN2c1ccc(-c2c(C)cc(C(F)(F)F)cc2O)nn1] | 435 1.34 U | 1H NMR (400 MHz, DMSO-d6, 30° C., mixture rotamers + atropoisomers) d ppm 1.35-1.67 (m, 4 H), 1.69-1.80 (m, 1 H), 1.80-1.92 (m, 1 H), 1.92- 2.24 (m, 2 H), 2.03 (s, 1.7 H), 2.06 (s, 1.3 H), 2.13 (s, 3 H), 2.54-2.64 (m, 0.45 H), 2.88-3.02 (m, 1 H), 3.05-3.16 (m, 0.55 H), 3.60-3.70 (m, 0.55 H), 3.93-4.03 (m, 0.45 H), 4.24 (br d, J = 12.7 Hz, 0.55 H), 4.33 (br d, J = 12.7 Hz, 0.9 H), 4.44-4.53 (m, 0.55 H), 4.54-4.67 (m, 1 H), 7.06 (s, 1 H), 7.11 (s, 1 H), 7.30-7.43 (m, 2 H), 10.15 (br s, 1 H) |
| 466 | 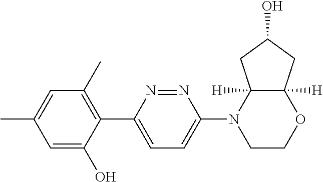 [SMILES: Cc1cc(C)c(-c2ccc(N3CCO[C@@H]4C[C@@H](O)C[C@@H]43)nn2)c(O)c1] | 342.3 1.61 Z3 | ^{1H} NMR (400 MHz, DMSO-d6): δ 9.40 (s, 1H), 7.35 (d, J = 9.6 Hz, 1H), 7.17 (d, J = 9.6 Hz, 1H), 6.58 (d, J = 4.0 Hz, 2H), 4.74 (d, J = 4.4 Hz, 1H), 4.69- 4.64 (m, 1H), 4.34 (s, 1H), 3.99-3.90 (m, 3H), 3.52 (t, J = 12.0 Hz, 1H), 3.08-3.03 (m, 1H), 2.22 (s, 3H), 2.03 (s, 3H), 1.94-1.86 (m, 1H), 1.79-1.72 (m, 2H), 1.24 (s, 1H) ppm |
| 467 | 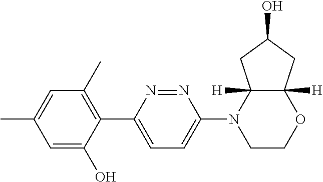 [SMILES: Cc1cc(C)c(-c2ccc(N3CCO[C@H]4C[C@H](O)C[C@H]43)nn2)c(O)c1] | 342.3 1.61 Z3 | 1H NMR (400 MHz, DMSO-d6): δ 9.40 (s, 1H), 7.35 (d, J = 9.6 Hz, 1H), 7.17 (d, J = 9.6 Hz, 1H), 6.58 (d, J = 4.6 Hz, 2H), 4.74 (d, J = 4.3 Hz, 1H), 4.67 (t, J = 10.2 Hz, 1H), 4.34 (s, 1H), 3.99-3.91 (m, 3H), 3.52 (t, J = 11.8 Hz, 1H), 3.08-3.03 (m, 1H), 2.22 (s, 3H), 2.03 (s, 3H), 1.94-1.86 (m, 1H), 1.79-1.72 (m, 2H), 1.24 (s, 1H) ppm. |
| 468 | 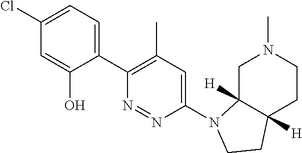 [SMILES: Cc1cc(N2CC[C@H]3CCN(C)C[C@H]32)nnc1-c1ccc(Cl)cc1O] | 359 0.78 U | 1H NMR (400 MHz, DMSO-d6, 30° C.) d ppm 1.66- 1.77 (m, 2 H), 1.84-2.05 (m, 3 H), 2.07 (br s, 3 H), 2.06-2.13 (m, 1 H), 2.17 (s, 3 H), 2.30-2.39 (m, 1 H), 2.52-2.57 (m partially hidden, 1 H), 3.06 (br dd, J = 6.0 and 10.5 Hz, 1 H), 3.34-3.43 (m, 1 H), 3.49-3.57 (m, 1 H), 4.15-4.25 (m, 1 H), 6.75 (s, 1 H), 6.91-6.97 (m, 2 H), 7.19 (br d, J = 7.8 Hz, 1 H), 10.27 (br s, 1 H) |
| 469 | 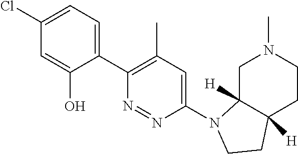 [SMILES: Cc1cc(N2CC[C@H]3CCN(C)C[C@H]32)nnc1-c1ccc(Cl)cc1O] | 359 0.78 U | 1H NMR (400 MHz, DMSO-d6, 30° C.) d ppm 1.66- 1.77 (m, 2 H), 1.85-2.05 (m, 3 H), 2.07 (s, 3 H), 2.06-2.15 (m, 1 H), 2.16 (s, 3 H), 2.29-2.39 (m, 1 H), 2.52- 2.56 (m partially hidden, 1 H), 3.06 (dd, J = 6.0 and 10.5 Hz, 1 H), 3.34-3.42 (m, 1 H), 3.50- 3.57 (m, 1 H), 4.15-4.24 (m, 1 H), 6.75 (s, 1 H), 6.91-6.96 (m, 2 H), 7.18 (br d, J = 7.8 Hz, 1 H), 10.29 (br s, 1 H) |
| 470 | 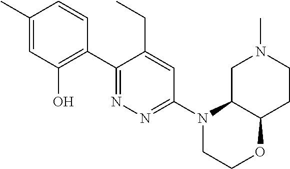 [SMILES: CCc1cc(N2CCO[C@@H]3CCN(C)C[C@@H]32)nnc1-c1ccc(C)cc1O] | 369 0.84 U | 1H NMR (400 MHz, DMSO-d6, 30° C.) d ppm 0.99 (t, J = 7.6 Hz, 3 H), 1.74-1.95 (m, 2 H), 2.04-2.16 (m, 1 H), 2.19 (s, 3 H), 2.28 (s, 3 H), 2.29-2.38 (m, 1 H), 2.42 (q, J = 7.6 Hz, 2 H), 2.52-2.58 (m partially hidden, 2 H), 3.14 (td, J = 3.8 and 12.7 Hz, 1 H), 3.62 (td, J = 3.3 and 11.8 Hz, 1 H), 3.73 (br q, J = 2.8 Hz, 1 H), 3.91 (br d, J = 12.7 Hz, 1 H), 4.04 (dd, J = 3.3 and 11.8 Hz, 1 H), 4.40-4.48 (m, 1 H), 6.70 (br d, J = 7.8 Hz, 1 H), 6.73 (br s, 1 H), 7.02 (d, J = 7.8 Hz, 1 H), 7.01 (s, 1 H), 9.47 (br s, 1 H) |
| 471 | 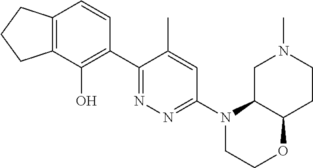 [SMILES: Cc1cc(N2CCO[C@@H]3CCN(C)C[C@@H]32)nnc1-c1ccc2c(c1O)CCC2] | 381 0.86 U | 1H NMR (400 MHz, DMSO-d6, 30° C.) d ppm 1.74- 1.94 (m, 2 H), 1.99-2.13 (m, 3 H), 2.15 (s, 3 H), 2.17 (s, 3 H), 2.27-2.35 (m, 1 H), 2.52-2.60 (m partially hidden, 2 H), 2.84 (t, J = 7.4 Hz, 2 H), 2.89 (t, J = 7.4 Hz, 2 H), 3.14 (td, J = 3.8 and 12.7 Hz, 1 H), 3.61 (td, J = 3.3 and 11.8 Hz, 1 H), 3.72 (br q, J = 2.8 Hz, 1 H), 3.91 (br d, J = 12.7 Hz, 1 H), 4.04 (dd, J = 3.3 and 11.8 Hz, 1 H), 4.36-4.43 (m, 1 H), 6.79 (d, J = 7.6 Hz, 1 H), 7.01 (d, J = 7.6 Hz, 1 H), 7.12 (s, 1 H), 9.39 (s, 1 H) |
| 472 | 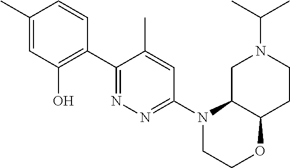 [SMILES: Cc1ccc(-c2nnc(N3CCO[C@@H]4CCN(C(C)C)C[C@@H]43)cc2C)c(O)c1] | 383 0.83 U | 1H NMR (400 MHz, DMSO-d6, 30° C.) d ppm 0.94 (br d, J = 12.1 Hz, 3 H), 0.95 (br d, J = 12.1 Hz, 3 H), 1.76-1.85 (m, 2 H), 2.10 (s, 3 H), 2.28 (s, 3 H), 2.30-2.37 (m, 1 H), 2.52-2.57 (m partially hidden, 2 H), 2.58-2.64 (m, 1 H), 2.66-2.76 (m, 1 H), 3.16 (td, J = 3.8 and 12.7 Hz, 1 H), 3.60 (td, J = 3.3 and 11.8 Hz, 1 H), 3.73 (br q, J = 2.8 Hz, 1 H), 3.90 (br d, J = 12.7 Hz, 1 H), 4.04 (br dd, J = 3.3 and 11.8 Hz, 1 H), 4.31-4.40 (m, 1 H), 6.71 (br d, J=7.8 Hz, 1 H), 6.73 (s, 1 H), 7.03-7.09 (m, 2 H), 9.66 (s, 1 H) |
| 473 | 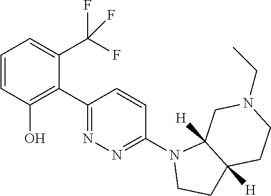 [SMILES: CCN1CC[C@@H]2CCN(c3ccc(-c4c(O)cccc4C(F)(F)F)nn3)[C@@H]2C1] | 393.3 2.4 E | ¹H NMR (400 MHz, DMSO) δ 10.08 (s, 1H), 7.44 (t, J = 8.0 Hz, 1H), 7.32-7.17 (m, 3H), 6.88 (d, J = 9.3 Hz, 1H), 4.21 (s, 1H), 3.56 (m, 1H), 3.42 (m, 1H), 3.15 (m, 1H), 2.73-2.61 (m, 1H), 2.44-2.30 (m, 3H), 2.22-2.01 (m, 2H), 1.92 (m, 2H), 1.81 (m, 1H), 1.72 (m, 1H), 1.00 (t, J = 7.1 Hz, 3H). |
| 474 | 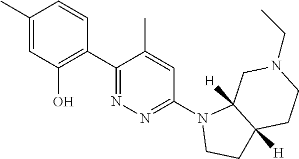 [SMILES: CCN1CC[C@@H]2CCN(c3cc(C)c(-c4ccc(C)cc4O)nn3)[C@@H]2C1] | 353.4 2.58 E | 1H NMR (400 MHz, DMSO-d6) δ (ppm): 9.71 (s, 1H), 7.05 (d, J = 7.6 Hz, 1H), 6.84-6.56 (m, 3H), 4.21 (s, 1H), 3.53 (t, J = 9.4 Hz, 1H), 3.40 (m, 1H), 3.15 (m, 1H), 2.64 (d, J = 11.3 Hz, 1H), 2.35 (p, J = 6.5 Hz, 3H), 2.28 (s, 3H), 2.09 (m, 4H), 2.06-2.00 (m, 1H), 1.92 (m, 2H), 1.82-1.67 (m, 2H), 0.99 (t, J = 7.1 Hz, 3H) |
| 475 | 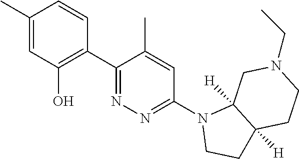 [SMILES: CCN1CC[C@H]2CCN(c3cc(C)c(-c4ccc(C)cc4O)nn3)[C@H]2C1] | 353.4 2.58 E | 1H NMR (400 MHz, DMSO-d6) δ (ppm): 9.71 (s, 1H), 7.05 (d, J = 7.6 Hz, 1H), 6.84-6.56 (m, 3H), 4.21 (s, 1H), 3.53 (t, J = 9.4 Hz, 1H), 3.40 (m, 1H), 3.15 (m, 1H), 2.64 (m, 1H), 2.35 (m, 3H), 2.28 (s, 3H), 2.17-2.06 (m, 4H), 2.06-2.00 (m, 1H), 1.92 (m, 2H), 1.82-1.67 (m, 2H), 0.99 (t, J = 7.1 Hz, 3H) |
| 476 | 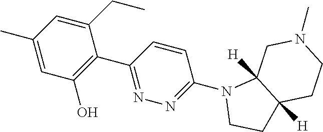 [SMILES: CCc1cc(C)cc(O)c1-c1ccc(N2CC[C@H]3CCN(C)C[C@H]32)nn1] | 353.4 2.66 E | 1H NMR (400 MHz, DMSO-d6) δ (ppm): 9.24 (s, 1H), 7.21 (d, J = 9.2 Hz, 1H), 6.87 (d, J = 9.3 Hz, 1H), 6.57 (br s, 2H), 4.20 (br, 1H), 3.55 (t, J = 9.4 Hz, 1H), 3.45-3.38 (m, 1H), 3.11-3.02 (m, 1H), 2.54-2.51 (m, 1H), 2.40-2.30 (m, 3H), 2.24 (s, 3H), 2.18 (s, 3H), 2.15-1.85 (m, 4H), 1.81-1.66 (m, 2H), 0.94 (t, J = 7.5 Hz, 3H) |
| 477 | 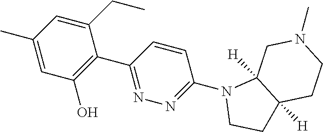 [SMILES: CCc1cc(C)cc(O)c1-c1ccc(N2CC[C@@H]3CCN(C)C[C@@H]32)nn1] | 353.4 2.66 E | 1H NMR (400 MHz, DMSO-d₆) δ (ppm): 9.25 (s, 1H), 7.21 (d, J = 9.3 Hz, 1H), 6.87 (d, J = 9.3 Hz, 1H), 6.61-6.54 (m, 2H), 4.20 (br s, 1H), 3.55 (t, J = 9.5 Hz, 1H), 3.45-3.37 (m, 1H), 3.11-3.00 (m, 1H), 2.59-2.53 (m, 1H), 2.42-2.29 (m, 3H), 2.24 (s, 3H), 2.17 (s, 3H), 2.15-1.85 (m, 4H), 1.82- 1.64 (m, 2H), 0.94 (t, J = 7.5 Hz, 3H) |
| 479 | 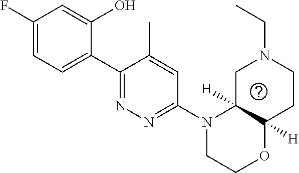 | 373 0.78 U | 1H NMR (400 MHz, DMSO-d6, 30° C.) d ppm 0.97 (t, J = 7.3 Hz, 3 H), 1.77-1.90 (m, 2 H), 2.04-2.13 (m, 1 H), 2.09 (s, 3 H), 2.29-2.42 (m, 3 H), 2.55- 2.65 (m, 2 H), 3.15 (td, J = 3.8 and 12.7 Hz, 1 H), 3.60 (td, J = 3.3 and 11.8 Hz, 1 H), 3.73 (br q, J = 2.8 Hz, 1 H), 3.91 (br d, J = 12.7 Hz, 1 H), 4.04 (dd, J = 3.3 and 11.8 Hz, 1 H), 4.35-4.46 (m, 1 H), 6.69- 6.76 (m, 2 H), 7.08 (s, 1 H), 7.21 (br t, J = 7.8 Hz, 1 H), 10.26 (br s, 1 H) |
| 480 | 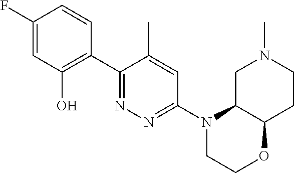 [SMILES: Cc1cc(N2CCO[C@@H]3CCN(C)C[C@@H]32)nnc1-c1ccc(F)cc1O] | 359 0.76 U | 1H NMR (400 MHz, DMSO-d6, 30° C.) d ppm 1.75- 1.93 (m, 2 H), 2.05-2.16 (m, 1 H), 2.09 (s, 3 H), 2.17 (s, 3 H), 2.26-2.35 (m, 1 H), 2.45-2.59 (m hidden, 2 H), 3.14 (td, J = 3.8 and 12.7 Hz, 1 H), 3.60 (td, J = 3.3 and 11.8 Hz, 1 H), 3.72 (br q, J = 2.8 Hz, 1 H), 3.91 (br d, J = 12.7 Hz, 1 H), 4.04 (dd, J = 3.3 and 11.8 Hz, 1 H), 4.33-4.46 (m, 1 H), 6.69- 6.76 (m, 2 H), 7.09 (s, 1 H), 7.21 (br t, J = 7.8 Hz, 1 H), 10.18-10.33 (m, 1 H) |
| 481 | 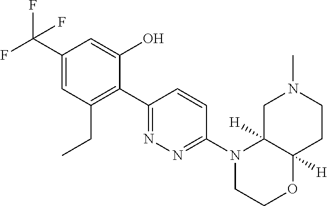 [SMILES: CCc1cc(C(F)(F)F)cc(O)c1-c1ccc(N2CCO[C@@H]3CCN(C)C[C@@H]32)nn1] | 423 1.06 U | 1H NMR (400 MHz, DMSO-d6, 30° C.) d ppm 0.98 (t, J = 7.5 Hz, 3 H), 1.77-1.93 (m, 2 H), 2.07-2.17 (m, 1 H), 2.19 (s, 3 H), 2.33-2.40 (m, 1 H), 2.40- 2.48 (m partially hidden, 2 H), 2.53-2.58 (m partially hidden, 2 H), 3.17 (td, J = 3.8 and 12.7 Hz, 1 H), 3.64 (td, J = 3.3 and 11.8 Hz, 1 H), 3.75 (br q, J = 2.8 Hz, 1 H), 3.95 (br d, J = 12.7 Hz, 1 H), 4.05 (dd, J = 3.3 and 11.8 Hz, 1 H), 4.40-4.48 (m, 1 H), 7.06 (d, J = 2.0 Hz, 1 H), 7.12 (d, J = 2.0 Hz, 1 H), 7.25 (d, J = 9.5 Hz, 1 H), 7.39 (d, J = 9.5 Hz, 1 H), 10.08 (br s, 1 H) |
| 482 | 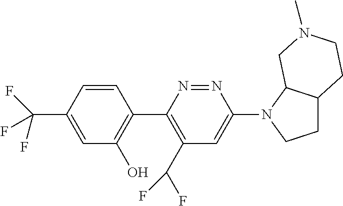 [SMILES: CN1CCC2CCN(c3cc(C(F)F)c(-c4ccc(C(F)(F)F)cc4O)nn3)C2C1] | 429 1.05 U | 1H NMR (400 MHz, DMSO-d6, 30° C.) d ppm 1.67- 1.84 (m, 2 H), 1.88-2.17 (m, 4 H), 2.18 (s, 3 H), 2.34-2.43 (m, 1 H), 2.54-2.58 (m partially hidden, 1 H), 3.01-3.11 (m, 1 H), 3.41-3.50 (m, 1 H), 3.58-3.67 (m, 1 H), 4.26-4.39 (m, 1 H), 6.83 (t, J = 54.3 Hz, 1 H), 6.97 (br s, 1 H), 7.22 (br s, 1 H), 7.26 (br d, J = 7.8 Hz, 1 H), 7.48 (d, J = 7.8 Hz, 1 H), 10.31-10.94 (m, 1 H) |
| 483 | 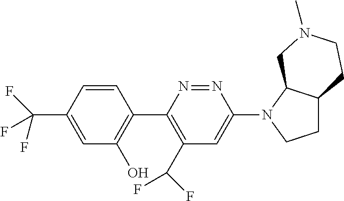 [SMILES: CN1CC[C@H]2CCN(c3cc(C(F)F)c(-c4ccc(C(F)(F)F)cc4O)nn3)[C@H]2C1] | 429 1.06 U | 1H NMR (400 MHz, DMSO-d6, 30° C.) d ppm 1.67- 1.83 (m, 2 H), 1.89-2.16 (m, 4 H), 2.18 (s, 3 H), 2.34-2.43 (m, 1 H), 2.53-2.58 (m partially hidden, 1 H), 3.01-3.10 (m, 1 H), 3.44-3.49 (m, 1 H), 3.58-3.66 (m, 1 H), 4.26-4.39 (m, 1 H), 6.84 (t, J = 54.3 Hz, 1 H), 6.97 (br s, 1 H), 7.21 (br s, 1 H), 7.24 (br d, J = 7.8 Hz, 1 H), 7.48 (d, J = 7.8 Hz, 1 H), 9.82-11.36 (m, 1 H) |
| 484 | 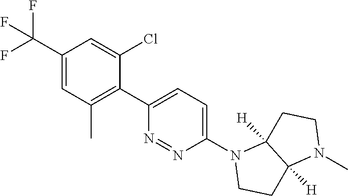 [SMILES: Cc1cc(C(F)(F)F)cc(Cl)c1-c1ccc(N2CC[C@H]3[C@@H]2CCN3C)nn1] | 379 0.9 U | 1H NMR (400 MHz, DMSO-d6, 100° C.) d ppm 1.47-1.57 (m, 1 H), 1.79-1.89 (m, 1 H), 1.94- 2.01 (m, 1 H), 2.16 (s, 3 H), 2.23-2.38 (m, 2 H), 2.32 (s, 3 H), 2.91-2.96 (m partially hidden, 1 H), 3.01 (t, J = 6.0 Hz, 1 H), 3.42-3.51 (m, 1 H), 3.78- 3.84 (m, 1 H), 4.44-4.50 (m, 1 H), 6.88 (d, J = 9.3 Hz, 1 H), 7.04-7.07 (m, 2 H), 7.30 (d, J = 9.3 Hz, 1 H), 9.70-9.94 (m, 1 H) |
| 485 | 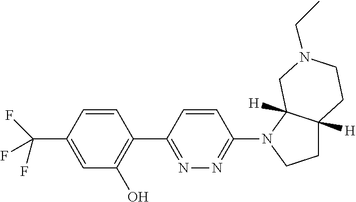 [SMILES: CCN1CC[C@@H]2CCN(c3ccc(-c4ccc(C(F)(F)F)cc4O)nn3)[C@@H]2C1] | 443.3 2.79 E | 1H NMR (400 MHz, DMSO) δ (ppm): 10.65 (s, 1H), 7.49 (d, J = 7.9 Hz, 1H), 7.26 (d, J = 8.0 Hz, 1H), 7.22 (s, 1H), 6.97 (s, 1H), 6.83 (t, J = 53.9 Hz, 1H), 4.33 (s, 1H), 3.64-3.57 (m, 1H), 3.48-3.39 (m, 1H), 3.16 (s, 1H), 2.64 (s, 1H), 2.45-2.39 (m, 1H), 2.35 (q, J = 7.2 Hz, 2H), 2.20-2.03 (m, 2H), 1.99-1.88 (m, 2H), 1.82 (t, J = 10.6 Hz, 1H), 1.72 (d, J = 13.5 Hz, 1H), 0.99 (t, J = 7.1 Hz, 3H). |
| 486 | 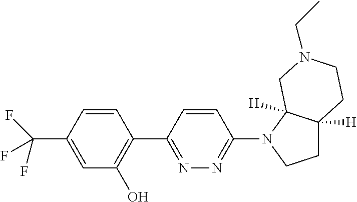 [SMILES: CCN1CC[C@H]2CCN(c3ccc(-c4ccc(C(F)(F)F)cc4O)nn3)[C@H]2C1] | 443.3 2.79 E | 1H NMR (400 MHz, DMSO) δ (ppm): 10.58 (s, 1H), 7.49 (d, J = 7.9 Hz, 1H), 7.27 (d, J = 8.0 Hz, 1H), 7.22 (s, 1H), 6.98 (d, J = 4.2 Hz, 1H), 6.84 (t, J = 54.1 Hz, 1H), 4.33 (s, 1H), 3.61 (t, J = 9.5 Hz, 1H), 3.44 (d, J = 8.6 Hz, 1H), 3.17 (s, 1H), 2.65 (s, 1H), 2.47-2.39 (m, 1H), 2.36 (q, J = 6.3 Hz, 2H), 2.23-2.01 (m, 2H), 2.01-1.88 (m, 2H), 1.82 (t, J = 10.6 Hz, 1H), 1.76-1.68 (m, 1H), 1.00 (t, J = 7.1 Hz, 3H). |
| 487 | 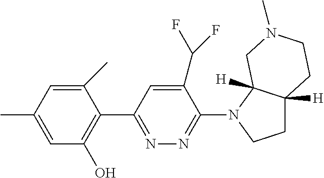 [SMILES: Cc1cc(C)c(-c2cc(C(F)F)c(N3CC[C@H]4CCN(C)C[C@H]43)nn2)c(O)c1] | 389.3 2.69 E | 1H NMR (400 MHz, DMSO) ¿ (ppm): 9.43 (s, 1H), 7.47 (s, 1H), 7.35 (t, J = 54.0 Hz, 1H), 6.60 (s, 2H), 4.59-4.50 (m, 1H), 3.89-3.77 (m, 1H), 3.57- 3.48 (m, 1H), 2.76 (dd, J = 11.7, 4.9 Hz, 1H), 2.38- 2.26 (m, 4H), 2.24 (s, 3H), 2.13 (s, 3H), 2.06 (s, 3H), 2.04-1.92 (m, 2H), 1.92-1.80 (m, 1H), 1.74- 1.62 (m, 1H). |
| 488 | 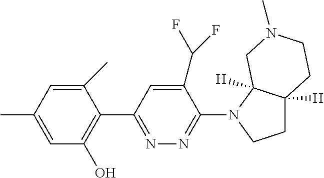 [SMILES: Cc1cc(C)c(-c2cc(C(F)F)c(N3CC[C@@H]4CCN(C)C[C@@H]43)nn2)c(O)c1] | 389.3 2.69 E | 1H NMR (400 MHz, DMSO) δ (ppm): 9.43 (s, 1H), 7.48 (s, 1H), 7.34 (t, J = 54.4 Hz, 1H), 6.60 (s, 2H), 4.56 (q, J = 6.0 Hz, 1H), 3.90-3.78 (m, 1H), 3.58- 3.47 (m, 1H), 2.91-2.71 (m, 1H), 2.43-2.27 (m, 4H), 2.24 (s, 3H), 2.18 (bs, 3H), 2.06 (s, 3H), 2.01- 1.93 (m, 2H), 1.86 (dd, J = 13.3, 6.6 Hz, 1H), 1.74- 1.65 (m, 1H). |
| 489 | 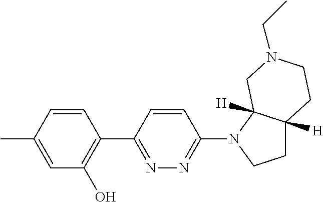 [SMILES: CCN1CC[C@@H]2CCN(c3ccc(-c4ccc(C)cc4O)nn3)[C@@H]2C1] | 389.4 2.56 E | 1H NMR (400 MHz, DMSO) δ (ppm): 9.84 (s, 1H), 7.14 (d, J = 7.5 Hz, 1H), 6.92 (s, 1H), 6.76 (m, 2H), 6.74 (t, J = 53.1 Hz, 1H), 4.32 (s, 1H), 3.65-3.56 (m, 1H), 3.46-3.39 (m, 1H), 3.16 (s, 1H), 2.69- 2.61 (m, 1H), 2.45-2.37 (m, 1H), 2.35 (q, J = 7.0 Hz, 2H), 2.29 (s, 3H), 2.22-2.01 (m, 2H), 2.00- 1.87 (m, 2H), 1.81 (t, J = 10.6 Hz, 1H), 1.72 (d, J = 14.4 Hz, 1H), 0.99 (t, J = 7.1 Hz, 3H). |
| 490 | 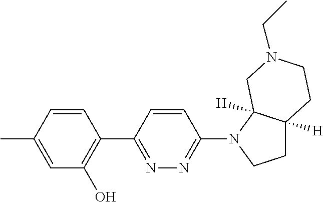 [SMILES: CCN1CC[C@H]2CCN(c3ccc(-c4ccc(C)cc4O)nn3)[C@H]2C1] | 389.4 2.56 E | 1H NMR (400 MHz, DMSO) δ 9.80 (s, 1H), 7.13 (d, J = 7.5 Hz, 1H), 6.92 (s, 1H), 6.75 (m, 2H), 6.73 (t, J = 53.2 Hz, 1H), 4.32 (s, 1H), 3.60 (t, J = 9.5 Hz, 1H), 3.45-3.38 (m, 1H), 3.17 (s, 1H), 2.70- 2.65 (m, 1H), 2.44-2.32 (m, 3H), 2.29 (s, 3H), 2.20-2.06 (m, 2H), 1.97-1.88 (m, 2H), 1.83 (s, 1H), 1.72 (d, J = 13.5 Hz, 1H), 0.99 (t, J = 7.1 Hz, 3H). |
| 491 | 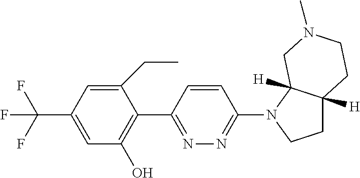 [SMILES: CCc1cc(C(F)(F)F)cc(O)c1-c1ccc(N2CC[C@H]3CCN(C)C[C@H]32)nn1] | 407.4 2.95 E | 1H NMR (400 MHz, DMSO-d6) δ (ppm): 10.14 (s, 1H), 7.30 (d, J = 9.3 Hz, 1H), 7.15-7.03 (m, 2H), 6.92 (d, J = 9.3 Hz, 1H), 4.22 (s, 1H), 3.57 (t, J = 9.5 Hz, 1H), 3.47-3.39 (m, 1H), 3.07 (br. s, 1H), 2.62-2.54 (m, 1H), 2.49-2.43 (m, 2H), 2.41- 2.32 (m, 1H), 2.18 (s, 3H), 2.15-1.87 (m, 4H), 1.84-1.64 (m, 2H), 0.98 (t, J = 7.5 Hz, 3H). |
| 492 | 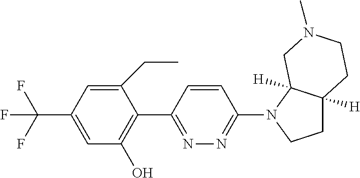 [SMILES: CCc1cc(C(F)(F)F)cc(O)c1-c1ccc(N2CC[C@@H]3CCN(C)C[C@@H]32)nn1] | 407.4 2.95 E | 1H NMR (400 MHz, DMSO-d6) δ (ppm): 9.98 (s, 1H), 7.14 (d, J = 9.3 Hz, 1H), 7.00-6.88 (m, 2H), 6.76 (d, J = 9.3 Hz, 1H), 4.07 (s, 1H), 3.41 (t, J = 9.5 Hz, 1H), 3.26 (t, J = 9.2 Hz, 1H), 2.97-2.88 (m, 1H), 2.42-2.37 (3, 1H), 2.30 (td, J = 7.5, 1.7 Hz, 2H), 2.26-2.15 (m, 1H), 2.03 (s, 3H), 1.99- 1.85 (m, 2H), 1.85-1.72 (m, 2H), 1.69-1.51 (m, 2H), 0.82 (t, J = 7.5 Hz, 3H). |
| 493 | 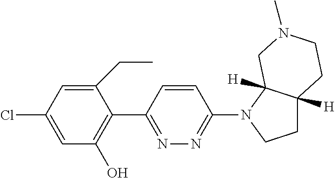 [SMILES: CCc1cc(Cl)cc(O)c1-c1ccc(N2CC[C@H]3CCN(C)C[C@H]32)nn1] | 374.3 2.81 E | ¹H NMR (400 MHz, DMSO-d₆) δ (ppm): 9.88 (s, 1H), 7.24 (d, J = 9.3 Hz, 1H), 6.89 (d, J = 9.2 Hz, 1H), 6.84 (d, J = 2.1 Hz, 1H), 6.80 (d, J = 2.1 Hz, 1H), 4.22 (br. s, 1H), 3.56 (t, J = 9.5 Hz, 1H), 3.45- 3.36 (m, 1H), 3.07 (s, 1H), 2.59-2.51 (s, 1H), 2.43-2.31 (m, 3H), 2.18 (s, 3H), 2.15-1.85 (m, 4H), 1.83 - 1.65 (m, 2H), 0.96 (t, J = 7.5 Hz, 3H). |
| 494 | 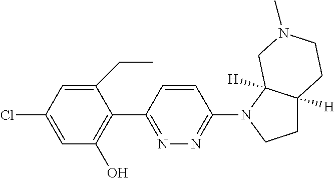 [SMILES: CCc1cc(Cl)cc(O)c1-c1ccc(N2CC[C@@H]3CCN(C)C[C@@H]32)nn1] | 374.3 2.79 E | ¹H NMR (400 MHz, DMSO-d₆) δ (ppm): 9.89 (s, 1H), 7.25 (d, J = 9.2 Hz, 1H), 6.89 (d, J = 9.3 Hz, 1H), 6.83 (d, J = 2.1 Hz, 1H), 6.80 (d, J = 2.1 Hz, 1H), 4.22 (br. s, 1H), 3.56 (t, J = 9.5 Hz, 1H), 3.44- 3.36 (m, 1H), 3.08 (s, 1H), 2.57-2.53 (m, 1H), 2.42-2.31 (m, 3H), 2.19 (s, 3H), 2.15-2.03 (m, 2H), 2.01-1.85 (m, 2H), 1.83-1.65 (m, 2H), 0.95 (t, J = 7.5 Hz, 3H). |
| 496 | 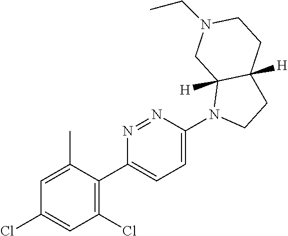 [SMILES: CCN1CC[C@@H]2CCN(c3ccc(-c4c(C)cc(Cl)cc4Cl)nn3)[C@@H]2C1] | 373 0.87 U | 1H NMR (400 MHz, DMSO-d6, 30° C.) δ ppm 1.00 (t, J = 7.1 Hz, 3 H) 1.67-1.75 (m, 1 H) 1.76-1.86 (m, 1 H) 1.86-1.99 (m, 2 H) 2.01-2.19 (m, 2 H) 2.06 (s, 3 H) 2.33-2.43 (m, 3 H) 2.59-2.67 (m, 1 H) 3.10-3.21 (m, 1 H) 3.35-3.44 (m, 1 H) 3.51- 3.59 (m, 1 H) 4.15-4.28 (m, 1 H) 6.80 (d, J = 2.1 Hz, 1 H) 6.83 (br d, J = 2.1 Hz, 1 H) 6.88 (br d, J = 9.4 Hz, 1 H) 7.27 (d, J = 9.4 Hz, 1 H) 9.94 (br s, 1 H) |
| 497 | 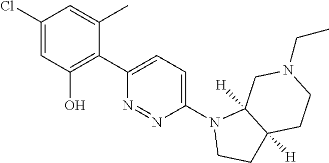 [SMILES: CCN1CC[C@H]2CCN(c3ccc(-c4c(C)cc(Cl)cc4O)nn3)[C@H]2C1] | 373 0.87 U | 1H NMR (400 MHz, DMSO-d6, 30° C.) δ ppm 0.99 (t, J = 7.1 Hz, 3 H) 1.66-1.75 (m, 1 H) 1.77-1.85 (m, 1 H) 1.86-1.98 (m, 2 H) 2.00-2.19 (m, 1 H) 2.06 (s, 3 H) 2.33-2.42 (m, 3 H) 2.61-2.68 (m, 1 H) 3.11-3.19 (m, 1 H) 3.35-3.43 (m, 1 H) 3.51- 3.60 (m, 1 H) 4.15-4.27 (m, 1 H) 6.80 (br d, J = 2.1 Hz, 1 H) 6.83 (br d, J = 2.1 Hz, 1 H) 6.88 (br d, J = 9.4 Hz, 1 H) 7.26 (d, J = 9.4 Hz, 1 H) 9.94 (br s, 1 H) |
| 498 | 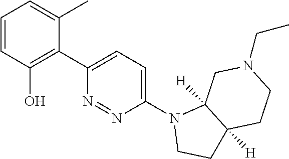 [SMILES: CCN1CC[C@H]2CCN(c3ccc(-c4c(C)cccc4O)nn3)[C@H]2C1] | 339 0.72 Z1 | 1H NMR (400 MHz, DMSO-d6, 30° C.) d ppm 0.99 (t, J = 7.1 Hz, 3 H), 1.68-1.74 (m, 1 H), 1.77-1.84 (m, 1 H), 1.86-1.98 (m, 2 H), 2.01-2.19 (m, 2 H), 2.06 (s, 3 H), 2.32-2.43 (m, 3 H), 2.61-2.69 (m, 1 H), 3.11-3.19 (m, 1 H), 3.36-3.44 (m, 1 H), 3.52- 3.59 (m, 1 H), 4.16-4.25 (m, 1 H), 6.71-6.78 (m, 2 H), 6.88 (br d, J = 9.4 Hz, 1 H), 7.08 (t, J = 7.8 Hz, 1 H), 7.27 (d, J = 9.4 Hz, 1 H), 9.44 (br s, 1 H) |
| 499 | 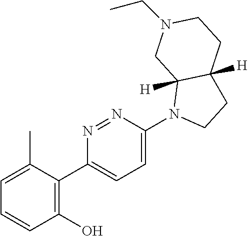 [SMILES: CCN1CC[C@@H]2CCN(c3ccc(-c4c(C)cccc4O)nn3)[C@@H]2C1] | 339 0.71 Z1 | 1H NMR (400 MHz, DMSO-d6, 30° C.) δ ppm 0.99 (t, J = 7.1 Hz, 3 H) 1.68-1.74 (m, 1 H) 1.77-1.84 (m, 1 H) 1.86-1.98 (m, 2 H) 2.01-2.18 (m, 2 H) 2.06 (s, 3 H) 2.32-2.43 (m, 3 H) 2.61-2.68 (m, 1 H) 3.11-3.19 (m, 1 H) 3.35-3.44 (m, 1 H) 3.52- 3.59 (m, 1 H) 4.17-4.25 (m, 1 H) 6.71-6.78 (m, 2 H) 6.88 (br d, J = 9.4 Hz, 1 H) 7.08 (t, J = 7.8 Hz, 1 H) 7.27 (d, J = 9.4 Hz, 1 H) 9.44 (br s, 1 H) |
| 500 | 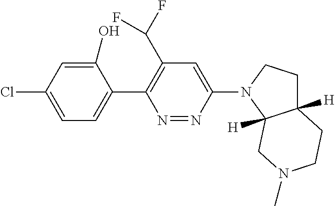 [SMILES: CN1CC[C@H]2CCN(c3cc(C(F)F)c(-c4ccc(Cl)cc4O)nn3)[C@H]2C1] | 395 0.99 U | 1H NMR (400 MHz, DMSO-d6, 30° C.) d ppm 1.66- 1.83 (m, 2 H), 1.90-2.16 (m, 4 H), 2.17 (s, 3 H), 2.34-2.43 (m, 1 H), 2.53-2.57 (m partially hidden, 1 H), 3.01-3.10 (m, 1 H), 3.39-3.49 (m, 1 H), 3.57-3.66 (m, 1 H), 4.24-4.37 (m, 1 H), 6.78 (t, J = 54.3 Hz, 1 H), 6.92-7.01 (m, 3 H), 7.27 (br d, J = 7.8 Hz, 1 H), 10.13-10.74 (m, 1 H) |
| 501 | 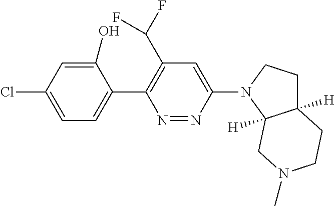 [SMILES: CN1CC[C@@H]2CCN(c3cc(C(F)F)c(-c4ccc(Cl)cc4O)nn3)[C@@H]2C1] | 395 0.99 U | 1H NMR (400 MHz, DMSO-d6, 30°° C.) 8 ppm 1.67- 1.83 (m, 2 H) 1.90-2.16 (m, 4 H) 2.17 (s, 3 H) 2.34-2.42 (m, 1 H) 2.53-2.57 (m partially hidden, 1 H) 3.01-3.11 (m, 1 H) 3.39-3.49 (m, 1 H) 3.56- 3.66 (m, 1 H) 4.24-4.38 (m, 1 H) 6.78 (t, J = 54.3 Hz, 1 H) 6.93-7.00 (m, 3 H) 7.27 (br d, J = 7.8 Hz, 1 H) 10.25-10.65 (m, 1 H) |
| 502 | 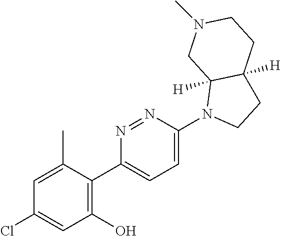 [SMILES: Cc1cc(Cl)cc(O)c1-c1ccc(N2CC[C@@H]3CCN(C)C[C@@H]32)nn1] | 359 0.84 U | 1H NMR (500 MHz, DMSO-d6, 27° C) d ppm 1.67- 1.81 (m, 2 H), 1.86-2.13 (m, 4 H), 2.05 (s, 3 H), 2.17 (s, 3 H), 2.33-2.40 (m, 1 H), 2.52-2.56 (m partially hidden, 1 H), 3.02-3.10 (m, 1 H), 3.38- 3.47 (m, 1 H), 3.52-3.59 (m, 1 H), 4.15-4.27 (m, 1 H), 6.80 (br d, J = 2.1 Hz, 1 H), 6.83 (br d, J = 2.1 Hz, 1 H), 6.89 (br d, J = 9.4 Hz, 1 H), 7.26 (d, J = 9.4 Hz, 1 H), 9.92-9.99 (m, 1 H) |
| 503 | 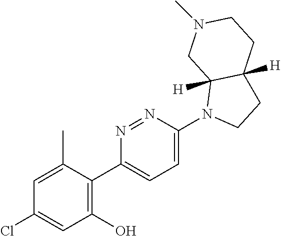 [SMILES: Cc1cc(Cl)cc(O)c1-c1ccc(N2CC[C@H]3CCN(C)C[C@H]32)nn1] | 359 0.84 U | 1H NMR (400 MHz, DMSO-d6, 30° C.) δ ppm 1.66- 1.82 (m, 2 H) 1.86-2.14 (m, 4 H) 2.05 (s, 3 H) 2.17 (s, 3 H) 2.33-2.41 (m, 1 H) 2.53-2.57 (m partially hidden, 1 H) 3.01-3.10 (m, 1 H) 3.38- 3.47 (m, 1 H) 3.52-3.59 (m, 1 H) 4.15-4.27 (m, 1 H) 6.80 (br d, J = 2.1 Hz, 1 H) 6.83 (br d, J = 2.1 Hz, 1 H) 6.89 (d, J = 9.4 Hz, 1 H) 7.26 (d, J = 9.4 Hz, 1 H) 9.88-10.09 (m, 1 H) |
| 504 | 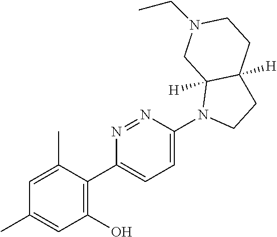 [SMILES: CCN1CC[C@H]2CCN(c3ccc(-c4c(C)cc(C)cc4O)nn3)[C@H]2C1] | 353 0.77 U | 1H NMR (400 MHz, DMSO-d6, 30° C.) δ ppm 0.99 (t, J = 7.1 Hz, 3 H) 1.67-1.75 (m, 1 H) 1.76-1.83 (m, 1 H) 1.85-1.98 (m, 2 H) 2.01-2.19 (m, 2 H) 2.04 (s, 3 H) 2.22 (s, 3 H) 2.31-2.43 (m, 3 H) 2.60- 2.69 (m, 1 H) 3.10-3.19 (m, 1 H) 3.34-3.43 (m, 1 H) 3.51-3.59 (m, 1 H) 4.16-4.24 (m, 1 H) 6.56 (br s, 1 H) 6.57 (br s, 1 H) 6.87 (br d, J = 9.4 Hz, 1 H) 7.25 (d, J = 9.4 Hz, 1 H) 9.39 (br s, 1 H) |
| 505 | 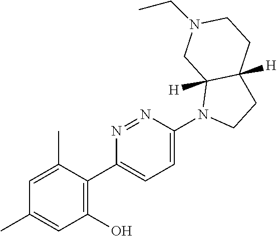 [SMILES: CCN1CC[C@@H]2CCN(c3ccc(-c4c(C)cc(C)cc4O)nn3)[C@@H]2C1] | 353 0.77 U | 1H NMR (400 MHz, DMSO-d6, 30° C.) δ ppm 0.99 (t, J = 7.1 Hz, 3 H) 1.67-1.74 (m, 1 H) 1.76-1.83 (m, 1 H) 1.85-1.98 (m, 2 H) 2.01-2.18 (m, 2 H) 2.04 (s, 3 H) 2.22 (s, 3 H) 2.31-2.42 (m, 3 H) 2.60- 2.68 (m, 1 H) 3.10-3.18 (m, 1 H) 3.34-3.43 (m, 1 H) 3.51-3.58 (m, 1 H) 4.15-4.24 (m, 1 H) 6.56 (br s, 1 H) 6.57 (br s, 1 H) 6.86 (br d, J = 9.4 Hz, 1 H) 7.25 (d, J = 9.4 Hz, 1 H) 9.40 (br s, 1 H) |
| 506 | 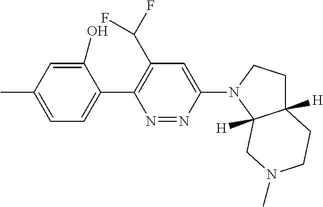 [SMILES: Cc1ccc(-c2nnc(N3CC[C@@H]4CCN(C)C[C@@H]43)cc2C(F)F)c(O)c1] | 375 0.94 U | 1H NMR (500 MHz, DMSO-d6, 27° C.) d ppm 1.67- 1.82 (m, 2 H), 1.89-2.14 (m, 4 H), 2.17 (s, 3 H), 2.29 (s, 3 H), 2.34-2.41 (m, 1 H), 2.53-2.56 (m partially hidden, 1 H), 3.01-3.10 (m, 1 H), 3.39- 3.48 (m, 1 H), 3.56-3.65 (m, 1 H), 4.25-4.37 (m, 1 H), 6.75 (t, J = 54.3 Hz, 2 H), 6.73-6.76 (m, 1 H), 6.92 (br s, 1 H), 7.13 (d, J = 7.8 Hz, 1 H), 9.80 (br s, 1 H) |
| 507 | 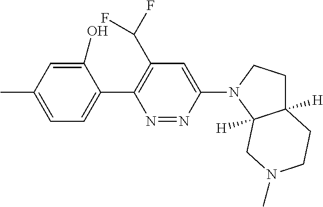 [SMILES: Cc1ccc(-c2nnc(N3CC[C@H]4CCN(C)C[C@H]43)cc2C(F)F)c(O)c1] | 375 0.94 U | 1H NMR (500 MHz, DMSO-d6, 27° C.) d ppm 1.66- 1.82 (m, 2 H), 1.89-2.15 (m, 4 H), 2.17 (s, 3 H), 2.29 (s, 3 H), 2.34-2.41 (m, 1 H), 2.52-2.56 (m partially hidden, 1 H), 3.01-3.10 (m, 1 H), 3.39- 3.47 (m, 1 H), 3.56-3.64 (m, 1 H), 4.24-4.38 (m, 1 H), 6.75 (t, J = 54.3 Hz, 1 H), 6.72-6.77 (m, 2 H), 6.92 (br s, 1 H), 7.13 (d, J = 7.8 Hz, 1 H), 9.80 (br s, 1 H) |
| 508 | 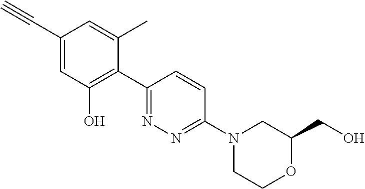 [SMILES: C#Cc1cc(C)c(-c2ccc(N3CCO[C@H](CO)C3)nn2)c(O)c1] | 230.1 0.76 S | 1H NMR (500 MHz, DMSO) δ 7.56 (d, J = 9.6 Hz, 1H), 7.38 (d, J = 9.6 Hz, 1H), 4.82 (t, J = 5.5 Hz, 1H), 4.24 (dt, J = 13.0, 2.0 Hz, 1H), 4.11-4.04 (m, 1H), 3.96 (ddd, J = 11.5, 3.6, 1.5 Hz, 1H), 3.57 (td, J = 11.6, 2.8 Hz, 1H), 3.54-3.48 (m, 2H), 3.47- 3.41 (m, 1H), 2.97 (ddd, J = 12.9, 11.7, 3.6 Hz, 1H), 2.76-2.71 (m, 1H) |
| 509 | 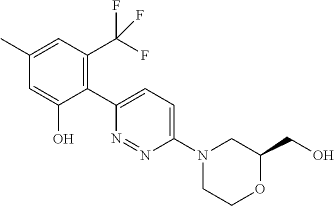 [SMILES: Cc1cc(O)c(-c2ccc(N3CCO[C@H](CO)C3)nn2)c(C(F)(F)F)c1] | 370.3 2.57 E | 1H NMR (400 MHz, DMSO-d6) 8 (ppm): 10.03 (s, 1H), 7.34 (d, J = 9.4 Hz, 1H), 7.27 (d, J = 9.5 Hz, 1H), 7.08 (d, J = 1.5 Hz, 1H), 7.02 (s, 1H), 4.86 (t, J = 5.5 Hz, 1H), 4.34 (d, J = 12.8 Hz, 1H), 4.14 (d, J = 12.8 Hz, 1H), 4.03-3.97 (m, 1H), 3.62 (td, J = 11.6, 2.8 Hz, 1H), 3.50-3.50 (m, 2H), 3.49-3.43 (m, 1H), 2.98 (td, J = 12.3, 3.5 Hz, 1H), 2.76-2.66 (m, 1H), 2.36 (s, 3H). |
| 510 | 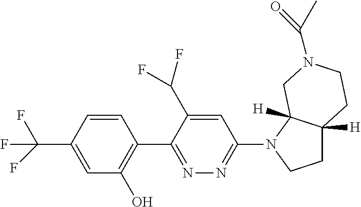 [SMILES: CC(=O)N1CC[C@@H]2CCN(c3cc(C(F)F)c(-c4ccc(C(F)(F)F)cc4O)nn3)[C@@H]2C1] | 457.3 2.75 E | 1H NMR (400 MHz, DMSO) δ (ppm): 10.63 (s, 1H), 7.51 (dd, J = 7.8, 2.8 Hz, 1H), 7.26 (d, J = 8.0 Hz, 1H), 7.22 (s, 1H), 7.10-6.69 (m, 1H), 6.86 (t, J = 54.3 Hz, 1H), 4.60 (dd, J = 13.1, 5.9 Hz, 0.5H), 4.37-4.27 (m, 0.5H), 4.26-4.15 (m, 0.5H), 4.01 (dd, J = 14.0, 5.3 Hz, 0.5H), 3.85 (d, J = 12.7 Hz, 0.5H), 3.68 (t, J = 9.4 Hz, 1H), 3.60 (dt, J = 13.5, 4.4 Hz, 0.5H), 3.46 (q, J = 8.9 Hz, 1H), 3.32-3.22 (m, 1H), 3.07 (t, J = 11.7 Hz, 0.5H), 2.70-2.63 (m, 0.5H), 2.5 (m, 25H, overintegration due to overlap with DMSO peak, expected 1H), 2.27-2.08 (m, 1H), 2.08-1.89 (m, 4.5H), 1.88-1.61 (m, 1.5H)- mixture of rotamers |
| 511 | 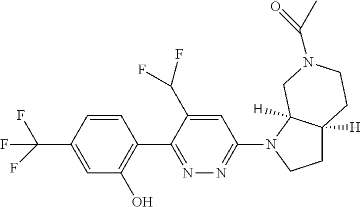 [SMILES: CC(=O)N1CC[C@H]2CCN(c3cc(C(F)F)c(-c4ccc(C(F)(F)F)cc4O)nn3)[C@H]2C1] | 457.3 2.75 E | 1H NMR (400 MHz, DMSO) δ (ppm): 10.62 (s, 1H), 7.51 (dd, J = 8.0, 2.8 Hz, 1H), 7.27 (d, J = 8.0 Hz, 1H), 7.22 (s, 1H), 7.10-6.70 (m, 1H), 6.86 (t, J = 54.4 Hz, 1H), 4.60 (dd, J = 13.1, 5.9 Hz, 0.5H), 4.32 (q, J = 7.1 Hz, 0.5H), 4.21 (s, 0.5H), 4.01 (dd, J = 14.2, 5.3 Hz, 0.5H), 3.85 (d, J = 13.0 Hz, 0.5H), 3.68 (t, J = 9.3 Hz, 1H), 3.60 (d, J = 13.9 Hz, 0.5H), 3.46 (q, J = 9.0 Hz, 1H), 3.28 (d, J = 10.9 Hz, 1H), 3.07 (t, J = 12.0 Hz, 0.5H), 2.70-2.63 (m, 0.5H), 2.5 (m, 29H, overintegration due to overlap with DMSO peak, expected 1H), 2.27-2.08 (m, 1H), 2.08-1.89 (m, 4.5H), 1.89-1.61 (m, 1.5H)- mixture of rotamers |
| 512 | 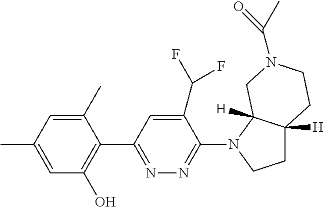 [SMILES: CC(=O)N1CC[C@@H]2CCN(c3nnc(-c4c(C)cc(C)cc4O)cc3C(F)F)[C@@H]2C1] | 417.4 2.74 E | 1H NMR (400 MHz, DMSO) 8 (ppm): 9.46 (s, 1H), 7.55-7.48 (m, 1H), 7.29 (td, J = 54.1, 25.8 Hz, 1H), 6.64-6.54 (m, 2H), 4.47-4.30 (m, 1H), 4.13- 3.62 (m, 3H), 3.57-3.42 (m, 2H), 2.91-2.79 (m, 1H), 2.57-2.44 (m, 41H; overintegration due to overlap with DMSO peak, expected 1H), 2.23 (s, 3H), 2.09-1.93 (m, 5H), 1.88-1.75 (m, 1H), 1.73- 1.61 (m, 1H), 1.49 (s, 2H), 1.47-1.36 (m, 1H) - Appears to be a mixture of rotamers |
| 513 | 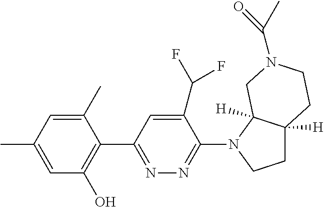 [SMILES: CC(=O)N1CC[C@H]2CCN(c3nnc(-c4c(C)cc(C)cc4O)cc3C(F)F)[C@H]2C1] | 417.4 2.74 E | 1H NMR (400 MHz, DMSO) δ (ppm): 9.45 (s, 1H), 7.56-7.47 (m, 1H), 7.48-7.11 (m, 1H), 6.64- 6.57 (m, 2H), 4.46-4.33 (m, 1H), 4.12-3.66 (m, 3H), 3.58-3.43 (m, 2H), 2.90-2.82 (m, 1H), 2.55- 2.47 (m, 44H; overintegration due to overlap with DMSO peak, expected 1H), 2.24 (s, 3H), 2.09- 1.99 (m, 4H), 1.97 (s, 1H), 1.86-1.77 (m, 1H), 1.72-1.63 (m, 1H), 1.50 (s, 2H), 1.49-1.36 (m, 1H) - Appears to be a mixture of rotamers. |
| 514 | 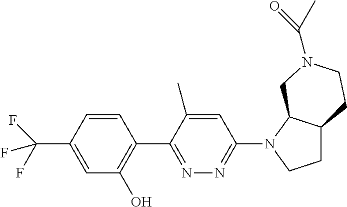 [SMILES: CC(=O)N1CC[C@H]2CCN(c3cc(C)c(-c4ccc(C(F)(F)F)cc4O)nn3)[C@H]2C1] | 421 1.10 U | 1H NMR (400 MHz, DMSO-d6, 100° C.) δ ppm 1.63-1.76 (m, 1 H) 1.83-1.99 (m, 1 H) 1.95 (br s, 3 H) 2.01-2.09 (m, 2 H) 2.12 (br s, 3 H) 2.51- 2.56 (m partially hidden, 1 H) 3.24-3.35 (m, 1 H) 3.38-3.49 (m, 1 H) 3.57-3.67 (m, 2 H) 3.78-4.70 (m, 2 H) 4.16-4.26 (m, 1 H) 6.75 (br s, 1 H) 7.19- 7.23 (m, 2 H) 7.42 (d, J = 7.8 Hz, 1 H) 9.99-10.34 (m, 1 H) |
| 515 | 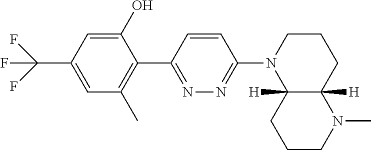 [SMILES: Cc1cc(C(F)(F)F)cc(O)c1-c1ccc(N2CCC[C@H]3[C@@H]2CCCN3C)nn1] | 407 1.00 U | 1H NMR (500 MHz, DMSO-d6, 27°C) d ppm 1.38- 1.48 (m, 2 H), 1.59-1.89 (m, 6 H), 2.13 (s, 3 H), 2.34 (br s, 3 H), 2.38-2.48 (m, 2 H), 2.84-2.97 (m, 2 H), 4.25 (br d, J = 12.7 Hz, 1 H), 4.51-4.64 (m, 1 H), 7.06 (br s, 1 H), 7.12 (br s, 1 H), 7.28 (d, J = 9.5 Hz, 1 H), 7.33 (d, J = 9.5 Hz, 1 H), 10.16 (br s, 1 H) |
| 516 | 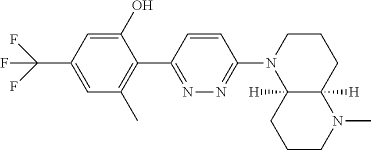 [SMILES: Cc1cc(C(F)(F)F)cc(O)c1-c1ccc(N2CCC[C@@H]3[C@H]2CCCN3C)nn1] | 407 1.00 U | 1H NMR (400 MHz, DMSO-d6, 30° C.) δ ppm 1.37- 1.49 (m, 2 H) 1.58-1.90 (m, 6 H) 2.13 (s, 3 H) 2.33 (br s, 3 H) 2.37-2.44 (m, 2 H) 2.83-2.97 (m, 2 H) 4.25 (br d, J = 12.7 Hz, 1 H) 4.51-4.60 (m, 1 H) 7.06 (br s, 1 H) 7.11 (br s, 1 H) 7.27 (d, J = 9.5 Hz, 1 H) 7.33 (d, J = 9.5 Hz, 1 H) 10.15 (br s, 1 H) |
| 517 | 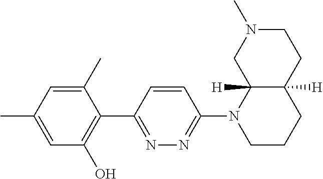 [SMILES: Cc1cc(C)c(-c2ccc(N3CCC[C@@H]4CCN(C)C[C@H]43)nn2)c(O)c1] | 353 0.87 U | 1H NMR (500 MHz, DMSO-d6, 27° C.) δ ppm 1.10- 1.18 (m, 1 H) 1.29-1.38 (m, 1 H) 1.63-1.81 (m, 6 H) 1.94-2.00 (m, 1 H) 2.04 (s, 3 H) 2.18 (s, 3 H) 2.22 (s, 3 H) 2.78-2.84 (m, 1 H) 3.13-3.19 (m, 1 H) 3.34-3.41 (m partially hidden, 2 H) 3.81-3.89 (m, 1 H) 6.57 (br s, 1 H) 6.59 (br s, 1 H) 7.13 (d, J = 9.4 Hz, 1 H) 7.33 (d, J = 9.4 Hz, 1 H) 9.46 (br s, 1 H) |
| 518 | 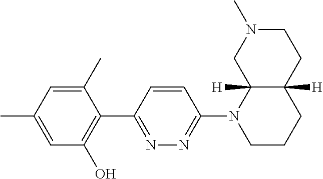 [SMILES: Cc1cc(C)c(-c2ccc(N3CCC[C@H]4CCN(C)C[C@H]43)nn2)c(O)c1] | 353 0.86 U | 1H NMR (500 MHz, DMSO-d6, 27° C.) δ ppm 1.38- 1.45 (m, 1 H) 1.47-1.57 (m, 2 H) 1.80-1.95 (m, 4 H) 1.99-2.09 (m, 1 H) 2.04 (s, 3 H) 2.17 (s, 3 H) 2.22 (s, 3 H) 2.24-2.30 (m, 1 H) 2.44-2.49 (m partially hidden, 2 H) 2.89 (td, J = 13.0, 2.7 Hz, 1 H) 4.16-4.22 (m, 1 H) 4.60-4.66 (m, 1 H) 6.56 (br s, 1 H) 6.57 (br s, 1 H) 7.19 (d, J = 9.5 Hz, 1 H) 7.28 (d, J = 9.5 Hz, 1 H) 9.43 (s, 1 H) |
| 519 | 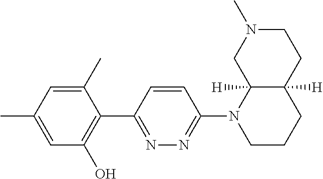 [SMILES: Cc1cc(C)c(-c2ccc(N3CCC[C@@H]4CCN(C)C[C@@H]43)nn2)c(O)c1] | 353 0.86 U | 1H NMR (500 MHz, DMSO-d6, 27° C.) δ ppm 1.38- 1.45 (m, 1 H) 1.47-1.57 (m, 2 H) 1.80-1.96 (m, 4 H) 2.00-2.09 (m, 1 H) 2.04 (s, 3 H) 2.17 (s, 3 H) 2.22 (s, 3 H) 2.24-2.30 (m, 1 H) 2.45-2.49 (m partially hidden, 2 H) 2.88 (td, J = 13.0, 2.7 Hz, 1 H) 4.15-4.23 (m, 1 H) 4.59-4.67 (m, 1 H) 6.56 (br s, 1 H) 6.57 (br s, 1 H) 7.19 (d, J = 9.5 Hz, 1 H) 7.28 (d, J = 9.5 Hz, 1 H) 9.44 (br s, 1 H) |
| 520 | 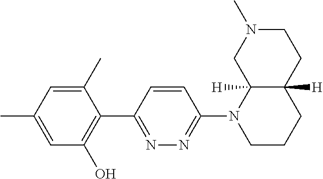 [SMILES: Cc1cc(C)c(-c2ccc(N3CCC[C@H]4CCN(C)C[C@@H]43)nn2)c(O)c1] | 353 0.88 U | 1H NMR (500 MHz, DMSO-d6, 27° C.) δ ppm 1.10- 1.18 (m, 1 H) 1.30-1.37 (m, 1 H) 1.63-1.81 (m, 6 H) 1.94-2.00 (m, 1 H) 2.04 (s, 3 H) 2.18 (s, 3 H) 2.22 (s, 3 H) 2.79-2.83 (m, 1 H) 3.14-3.18 (m, 1 H) 3.34-3.40 (m partially hidden, 2 H) 3.83-3.88 (m, 1 H) 6.57 (br s, 1 H) 6.59 (br s, 1 H) 7.13 (d, J = 9.4 Hz, 1 H) 7.33 (d, J = 9.4 Hz, 1 H) 9.49 (br s, 1 H) |
| 521 | 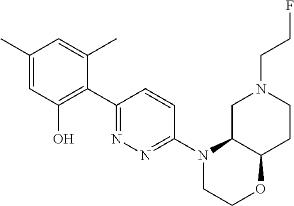 [SMILES: Cc1cc(C)c(-c2ccc(N3CCO[C@@H]4CCN(CCF)C[C@@H]43)nn2)c(O)c1] | 387 0.83 U | 1H NMR (400 MHz, DMSO-d6, 30° C.) δ ppm 1.77- 1.92 (m, 2 H) 2.03 (s, 3 H) 2.22 (s, 3 H) 2.23- 2.30 (m, 1 H) 2.52-2.58 (m partially hidden, 1 H) 2.58-2.72 (m, 4 H) 3.16 (td, J = 12.7, 3.8 Hz, 1 H) 3.63 (td, J = 11.8, 3.3 Hz, 1 H) 3.75 (q, J = 2.8 Hz, 1 H) 3.93 (br d, J = 12.7 Hz, 1 H) 4.05 (dd, J = 11.8, 3.3 Hz, 1 H) 4.38-4.44 (m, 1 H) 4.43-4.61 (m, 2 H) 6.57 (br s, 1 H) 6.58 (br s, 1 H) 7.20 (d, J = 9.5 Hz, 1 H) 7.34 (d, J = 9.5 Hz, 1 H) 9.38 (br s, 1 H) |
| 522 | 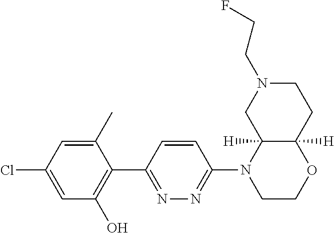 [SMILES: Cc1cc(Cl)cc(O)c1-c1ccc(N2CCO[C@@H]3CCN(CCF)C[C@@H]32)nn1] | 407 0.92 U | 1H NMR (400 MHz, DMSO-d6, 30° C.) δ ppm 1.76- 1.92 (m, 2 H) 2.05 (s, 3 H) 2.20-2.30 (m, 1 H) 2.52-2.59 (m partially hidden , 1 H) 2.59-2.72 (m, 4 H) 3.16 (td, J = 12.7, 3.8 Hz, 1 H) 3.63 (td, J = 11.8, 3.3 Hz, 1 H) 3.75 (q, J = 2.8 Hz, 1 H) 3.94 (br d, J = 12.7 Hz, 1 H) 4.05 (dd, J = 11.8, 3.3 Hz, 1 H) 4.39-4.61 (m, 3 H) 6.81 (br d, J = 2.1 Hz, 1 H) 6.84 (br d, J = 2.1 Hz, 1 H) 7.22 (d, J = 9.5 Hz, 1 H) 7.36 (d, J = 9.5 Hz, 1 H) 9.96 (br s, 1 H) |
| 523 | 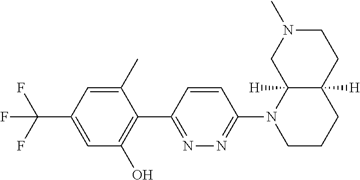 [SMILES: Cc1cc(C(F)(F)F)cc(O)c1-c1ccc(N2CCC[C@@H]3CCN(C)C[C@@H]32)nn1] | 407 1.05 U | 1H NMR (500 MHz, DMSO-d6, 27° C.) δ ppm 1.40- 1.45 (m, 1 H) 1.49-1.57 (m, 2 H) 1.82- 1.96 (m, 4 H) 2.03-2.09 (m, 1 H) 2.13 (s, 3 H) 2.18 (s, 3 H) 2.26-2.31 (m, 1 H) 2.46-2.49 (m partially hidden, 1 H) 2.51-2.54 (m partially hidden, 1 H) 2.87- 2.94 (m, 1 H) 4.19-4.25 (m, 1 H) 4.63-4.69 (m, 1 H) 7.06 (br s, 1 H) 7.11 (br s, 1 H) 7.24 (d, J = 9.5 Hz, 1 H) 7.35 (d, J = 9.5 Hz, 1 H) 10.20 (br s, 1 H) |
| 524 | 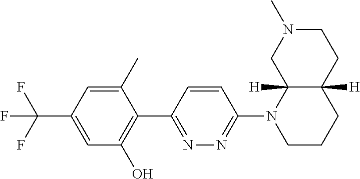 [SMILES: Cc1cc(C(F)(F)F)cc(O)c1-c1ccc(N2CCC[C@H]3CCN(C)C[C@H]32)nn1] | 407 1.05 U | 1H NMR (500 MHz, DMSO-d6, 27° C.) δ ppm 1.40- 1.45 (m, 1 H) 1.49-1.58 (m, 2 H) 1.82-1.96 (m, 4 H) 2.03-2.09 (m, 1 H) 2.13 (s, 3 H) 2.18 (s, 3 H) 2.26-2.31 (m, 1 H) 2.46-2.49 (m partially hidden, 1 H) 2.51-2.54 (m partially hidden, 1 H) 2.87- 2.94 (m, 1 H) 4.19-4.25 (m, 1 H) 4.63-4.71 (m, 1 H) 7.05 (br s, 1 H) 7.11 (br s, 1 H) 7.24 (d, J = 9.5 Hz, 1 H) 7.35 (d, J = 9.5 Hz, 1 H) 10.18 (br s, 1 H) |
| 525 | 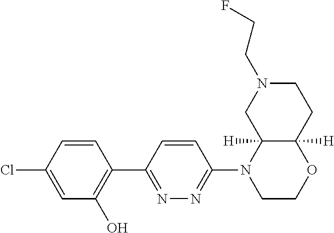 [SMILES: Oc1cc(Cl)ccc1-c1ccc(N2CCO[C@@H]3CCN(CCF)C[C@@H]32)nn1] | 393 1.03 U | 1H NMR (400 MHz, DMSO-d6, 30° C.) δ ppm 1.76- 1.92 (m, 2 H) 2.21-2.30 (m, 1 H) 2.52-2.58 (m partially hidden, 1 H) 2.58-2.71 (m, 4 H) 3.19 (td, J=12.7, 3.8 Hz, 1 H) 3.63 (td, J = 11.8, 3.3 Hz, 1 H) 3.76 (q, J = 2.8 Hz, 1 H) 3.96 (br d, J = 12.7 Hz, 1 H) 4.05 (dd, J = 11.8, 3.3 Hz, 1 H) 4.41-4.61 (m, 3 H) 6.98 (dd, J = 8.4, 2.1 Hz, 1 H) 7.02 (d, J = 2.1 Hz, 1 H) 7.51 (d, J = 9.8 Hz, 1 H) 7.93 (d, J = 8.4 Hz, 1 H) 8.26 (d, J = 9.8 Hz, 1 H) 13.84 (br s, 1 H) |
| 526 | 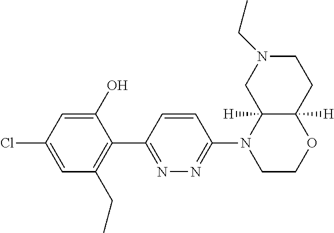 [SMILES: CCc1cc(Cl)cc(O)c1-c1ccc(N2CCO[C@@H]3CCN(CC)C[C@@H]32)nn1] | 403 0.99 U | 1H NMR (400 MHz, DMSO-d6, 30° C.) δ ppm 0.91- 1.01 (m, 6 H) 1.76-1.90 (m, 2 H) 2.03-2.14 (m, 1 H) 2.32-2.41 (m, 5 H) 2.55-2.65 (m, 2 H) 3.16 (td, J = 12.7, 3.8 Hz, 1 H) 3.63 (td, J = 11.8, 3.3 Hz, 1 H) 3.76 (br q, J = 2.8 Hz, 1 H) 3.94 (br d, J = 12.7 Hz, 1 H) 4.05 (dd, J = 11.8, 3.3 Hz, 1 H) 4.36-4.44 (m, 1 H) 6.81 (d, J = 2.1 Hz, 1 H) 6.84 (d, J = 2.1 Hz, 1 H) 7.21 (d, J = 9.5 Hz, 1 H) 7.34 (d, J = 9.5 Hz, 1 H) 9.89 (br s, 1 H) |
| 527 | 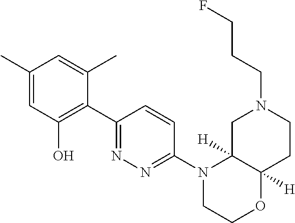 [SMILES: Cc1cc(C)c(-c2ccc(N3CCO[C@@H]4CCN(CCCF)C[C@@H]43)nn2)c(O)c1] | 401 0.86 U | 1H NMR (400 MHz, DMSO-d6, 30° C.) δ ppm 1.72- 1.92 (m, 4 H) 2.03 (s, 3 H) 2.08-2.17 (m, 1 H) 2.22 (s, 3 H) 2.37-2.45 (m, 3 H) 2.56-2.64 (m, 2 H) 3.16 (td, J = 12.7, 3.8 Hz, 1 H) 3.63 (td, J = 11.8, 3.3 Hz, 1 H) 3.75 (q, J = 2.8 Hz, 1 H) 3.93 (br d, J = 12.7 Hz, 1 H) 4.05 (dd, J = 11.8, 3.3 Hz, 1 H) 4.36-4.44 (m, 1 H) 4.46 (dt, J = 47.4, 6.0 Hz, 2 H) 6.57 (s, 1 H) 6.58 (s, 1 H) 7.19 (d, J = 9.5 Hz, 1 H) 7.34 (d, J = 9.5 Hz, 1 H) 9.38 (s, 1 H) |
| 528 | 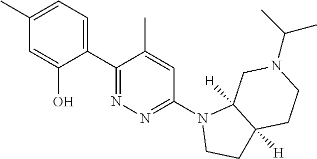 [SMILES: Cc1ccc(-c2nnc(N3CC[C@@H]4CCN(C(C)C)C[C@@H]43)cc2C)c(O)c1] | 367,4 2.66 E | 1H NMR (400 MHz, CDCl3) δ (ppm): 7.29 (d, J = 7.9 Hz, 1H), 6.91 (s, 1H), 6.71 (d, J = 8.0 Hz, 1H), 6.65 (s, 1H), 4.21 (s, 1H), 3.71 (s, 1H), 3.53 (s, 1H), 3.12 (s, 1H), 2.91-2.76 (m, 1H), 2.68 (s, 1H), 2.52- 2.48 (m, 1H), 2.45 (s, 3H), 2.34 (s, 3H), 2.32- 2.38 (m, 1H), 2.24-1.89 (m, 4H), 1.84-1.75 (m, 1H), 1.05 (s, 6H) |

### EXAMPLES

The examples which follow describe the preparation of certain compounds in accordance with the disclosure. The numbers of the compounds of formula (I) exemplified below match those given above. All reactions are performed under inert atmosphere, unless otherwise stated. These examples are not limitative, and merely illustrate the present disclosure.

Analytical data (LC-MS and ¹H RMN) were not repeated in the examples for the final compounds as they are already disclosed in Table 1.

The following abbreviations are used:
ACN or MeCN Acetonitrile
AcOK Potassium acetate
Aq. Aqueous
Ar Argon
BBr₃ Boron tribromide
BH₃-THF Borane tetrahydrofuran complex
BippyPhos 5-(di-tert-Butylphosphino)-1′,3′,5′-triphenyl-1′H-1,4′-bipyrazole
Boc Tert-butyloxycarbonyl
Boc₂O Di-tert-butyl Dicarbonate
BrettPhos Pd G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
BuOH Butanol
CO₂ Carbon dioxide
CV Column Volume
DCM or CH₂Cl₂ Dichloromethane
CbzCl Benzyl chloroformate
CDCl₃ Deuterochloroform
CyJohnPhos Chloro(2-dicyclohexylphosphino-1,1′-biphenyl)[2-(2′-amino-1,1′-biphenyl)]palladium(II)
DIAD Diisopropyl azodicarboxylate
DIPE Diisopropylether
DIPEA Diisopropylethylamine
DMAP 4-Dimethylamino Pyridine
DME Dimethylether
DMF N,N-dimethylformamide
DMSO-d₆ Hexadeuterodimethyl sulfoxide
EA Ethyl acetate
Et₃N Triethylamine
EtOH Ethanol
EtOAc or AcOEt Ethylacetate
GPhos Pd G3 Methanesulfonato[Dicyclohexyl[3-(1,1-dimethylethoxy)-6-methoxy-2′,6′-bis(1-methylethyl)[1,1′-biphenyl]-2-yl]phosphine](2′-amino-1,1′-biphenyl-2-yl)palladium(II)
H₂ Hydrogen
H₂O Water
HATU Hexafluorophosphate Azabenzotriazole Tetramethyl Uronium
HBpin Pinacolborane
HCl Hydrogen chloride
HCOOH Formic acid
HCl Hydrochloric acid
Hept. Heptane
HMDS Bis(trimethylsilyl)amine
I₂ Iodine
IL 1b Interleukin 1 beta
iPrNH₂ Isopropylamine
iPrOH Isopropanol
K₂CO₃ Potassium carbonate
K₃PO₄ Tripotassium phosphate
LC Liquid chromatography
LiOH Lithium hydroxide
LiOH·H₂O Lithium hydroxide monohydrate
LiHMDS Lithium bis(trimethylsilyl)amide
m-CPBA m-Chloroperbenzoic acid
Me-THF 2-methyl tetrahydrofuran
MeOD Tetradeuteromethanol
MeOH Methanol
MgSO₄ Magnesium sulfate
N₂ Nitrogen
NaBD₄ Sodium borodeuteride
NaBH₄ Sodium borohydride
NaBH₃CN Sodium cyanoborohydride
NaCl Sodium chloride
NaH Sodium hydride
NaHCO₃Sodium bicarbonate
Na₂CO₃ Sodium carbonate
Na₂SO₄ Sodium sulfate
Na₂S₂O₃ Sodium thiosulfate
NaO^{t}Bu Sodium tert-butoxide
NaOH Sodium hydroxide
NaOTMS Sodium trimethylsiloxide
NBS N-bromosuccinimide
NEt₃ or TEA Triethylamine
NH₃·H₂O Ammonia aqueous
NH₄HCO₃ Ammonium hydrogen carbonate
NMP N-methyl-2-pyrrolidone
NH₄Cl Ammonium chloride
NH₄OH Ammonium hydroxide
Pd(PPh₃)₄ Tetrakis(triphenylphosphine)palladium
Pd(dppf)Cl₂ or PdCl₂(dppf) [1,1′-Bis(diphenylphosphino)ferrocene]palladium(II) dichloride
Pd-118 or Pd(dtbpf)Cl₂ or PdCl₂(dtbpf) [1,1′-Bis(di-tert-butylphosphino)ferrocene]dichloropalladium(II)
Pd₂(dba)₃ Tris(dibenzylideneacetone)dipalladium (0)
Pd(OAc)₂ Palladium (II) acetate
Pd(OH)₂ Palladium hydroxide
Pd/C Palladium on carbon
PPh₃ Triphenyl phosphine
TBAF Tetra-n-butylammonium fluoride
TFA Trifluoroacetic acid
THF Tetrahydrofuran
TNF-alpha or TNF-α Tumor necrosis factor-alpha or Tumor necrosis factor-α
rt Room temperature
Rt Retention time
RuPhos 2-Dicyclohexylphosphino-2′,6′-diisopropoxybiphenyl;
RuphosPdG₃ Methanesulfonato(2-dicyclohexylphosphino-2′,6′-di-1-propoxy-1,1′-biphenyl)(2′-amino-1,1′-biphenyl-2-yl-palladium(II)
SFC Supercritical fluid chromatography
SiOH Silicon hydroxide
SiO₂ Silicon dioxide
Xphos 2-Dicyclohexylphosphino-2′,4′,6′-triisopropylbiphenyl
XPhosPdG₂ Chloro(2-dicyclohexylphosphino-2′,4′,6′-triisopropyl-1,1′-biphenyl)[2-(2′-amino-1,1′-biphenyl)]palladium(II)
° C. Degree Celsius
ml Milliliter(s)
mmol Millimole(s)
min Minute(s)
μmol Micromole(s)
μl Microliter(s)
h Hour(s)

### Analytical Methods Used

### NMR:

The proton magnetic resonance spectra (¹H NMR), as described below, are recorded at 400 MHz or 500 MHz, mainly in DMSO-d₆, using the DMSO peak as reference. The chemical shifts (δ) are expressed in parts per million (ppm). The signals observed are expressed as follows: s=singlet; d=doublet; t=triplet; m=multiplet or br s=broad singlet; br m=broad multiplet

### LCMS:

The LCMS characteristics, as described below, indicates the different high-performance liquid chromatography analytical methods used.

### LCMS Final Compounds:

### Method A:

System Waters UPLC/SQD; ionization:electrospray in positive and/or negative mode (ES+/−); Column: ACQUITY CORTECS C18+ 1.7 μm 2.1×50 mm; Column temperature: 40° C.; Flow: 1.0 ml/min; Solvents: A=H₂O (0.1% formic acid); B=ACN (0.1% formic acid)

### Gradient:

| (min) | % A | % B |
|---|---|---|
| 0 | 98 | 2 |
| 2.00 | 0 | 100 |
| 2.60 | 0 | 100 |
| 2.70 | 98 | 2 |
| 3.00 | 98 | 2 |

### Method B:

System Waters UPLC-SQD2; ionization:electrospray in positive and/or negative mode (ES+/−); Column: ACQUITY CSH C18+ 1.7 μm 2.1×50 mm; Column temperature: 60° C. Flow: 1.0 ml/min; Solvents: A=H₂O (0.1% formic acid); B=ACN (0.1% formic acid)

### Gradient:

| (min) | % A | % B |
|---|---|---|
| 0 | 97 | 3 |
| 2.10 | 0 | 100 |
| 2.45 | 0 | 100 |
| 2.50 | 97 | 3 |

### Method C:

System SHIMADZU LCMS-2020 Qarray; ionization:electrospray in positive and/or negative mode (ES+/−); Column: HALO C18 2.7 μm 3.0×30 mm; Column temperature: 50° C. Flow: 1.5 ml/min; Solvents: A=H₂O (0.0375% TFA); B=ACN (0.01875% TFA)

### Gradient:

| (min) | % A | % B |
|---|---|---|
| 0 | 95 | 5 |
| 0.5 | 5 | 95 |
| 0.8 | 5 | 95 |
| 0.81 | 95 | 5 |
| 1.05 | 95 | 5 |

### Method D:

System SHIMADZU LCMS-2020 Qarray; ionization:electrospray in positive and/or negative mode (ES+/−); Column: HALO C18 5 μm 3.0×30 mm; Column temperature: 50° C. Flow: 2 ml/min; Solvents: A=H₂O (0.0375% TFA); B=ACN (0.01875% TFA) Gradient:

| (min) | % A | % B |
|---|---|---|
| 0 | 95 | 5 |
| 0.4 | 5 | 95 |
| 0.75 | 5 | 95 |
| 0.76 | 95 | 5 |
| 1.05 | 95 | 5 |

### Method E:

System Waters UPLC-DAD and QDa; ionization:electrospray in positive and/or negative mode (ES+/−); Column: ACQUITY BEH C18 1.7 μm 2.1×100 mm; Column temperature: 40° C.; Flow: 0.343 ml/min; Solvents: A=H₂O 95%, ACN 5% (0.5 g/l AcNH₄); B=ACN

### Gradient:

| (min) | % A | % B |
|---|---|---|
| 0 | 84.2 | 15.8 |
| 0.49 | 84.2 | 15.8 |
| 2.67 | 10.5 | 89.5 |
| 4.61 | 10.5 | 89.5 |
| 5.34 | 84.2 | 15.8 |
| 6.07 | 84.2 | 15.8 |

### Method F:

System Waters UPLC-DAD and ZQ; ionization:electrospray in positive and/or negative mode (ES+/−); Column: Alliance YMC C18 5 μm 4.6×75 mm; Column temperature: 30° C. Flow: 1 ml/min; Solvents: A=H₂O (0.2% NH₄HCO₃); B=ACN

### Gradient:

| (min) | % A | % B |
|---|---|---|
| 0 | 90 | 10 |
| 13 | 0 | 100 |
| 13.5 | 0 | 100 |
| 13.8 | 90 | 10 |
| 15 | 90 | 10 |

### Method Z1:

System Waters UPLC-SQD2; ionization:electrospray in positive and/or negative mode (ES+/−); Column: ACQUITY CSH C18 1.7 μm 2.1×50 mm; Column temperature: 60° C.; Flow: 1.0 ml/min; Solvents: A=H₂O (0.1% formic acid); B=ACN (0.1% formic acid)

### Gradient:

| (min) | % A | % B |
|---|---|---|
| 0 | 97 | 3 |
| 8.6 | 1 | 99 |
| 9.6 | 1 | 99 |
| 9.8 | 97 | 3 |
| 10 | 97 | 3 |

### Method Z2:

System Waters UPLC-XEVO G2S-QTOF; ionization:electrospray in positive and/or negative mode (ES+/−); Column: ACQUITY BEH C4—1.7 μm-2.1×150 mm; Column temperature: 35° C.; Flow: 0.5 ml/min; Solvents: A=H2O (0.1% formic acid+0.2% TEA); B=CH₃CN (0.1% formic acid+0.2% TEA)

### Gradient:

| (min) | % A | % B |
|---|---|---|
| 0 | 90 | 10 |
| 1 | 80 | 20 |
| 5.5 | 1 | 98 |
| 8.5 | 1 | 98 |
| 10 | 90 | 10 |

### Method Z3:

System Agilent Technologies 1200 Series; ionization:electrospray in positive and/or negative mode (ES+/−); Column: XBridge C18 3.5 μm 4.6×50 mm; Column temperature: 45° C.; Flow: 1.8 ml/min; Solvents: A=H₂O (10 mM NH₄HCO₃); B=ACN

### Gradient:

| (min) | % A | % B |
|---|---|---|
| 0 | 95 | 5 |
| 1.4 | 5 | 95 |
| 3.0 | 5 | 95 |

### LCMS, Intermediates

### Method G:

System Waters UPLC-SQD2; ionization:electrospray in positive and/or negative mode (ES+/−); Column: ACQUITY CSH C18 1.7 μm 2.1×50 mm; Column temperature: 50° C. Flow: 0.8 ml/min; Solvents: A=H₂O (0.1% formic acid); B=ACN (0.1% formic acid)

### Gradient:

| (min) | % A | % B |
|---|---|---|
| 0 | 95 | 5 |
| 0.20 | 95 | 5 |
| 3.60 | 2 | 98 |
| 3.70 | 2 | 98 |
| 4.10 | 95 | 5 |
| 5.00 | 95 | 5 |

### Method H:

System Waters UPLC-SQD2; ionization:electrospray in positive and/or negative mode (ES+/−); Column: ACQUITY CSH C18 1.7 μm 2.1×30 mm; Column temperature: 35° C. Flow: 0.7 ml/min; Solvents: A=H₂O (0.1% formic acid); B=ACN (0.1% formic acid)

### Gradient:

| (min) | % A | % B |
|---|---|---|
| 0 | 95 | 5 |
| 0.18 | 95 | 5 |
| 3.20 | 1 | 99 |
| 3.40 | 1 | 99 |
| 3.55 | 95 | 5 |
| 4.00 | 95 | 5 |

### Method I:

System Waters UPLC/SQD; ionization:electrospray in positive and/or negative mode (ES+/−); Column: ACQUITY CORTECS C18 1.6 μm 2.1×50 mm; Column temperature: 55° C.; Flow: 0.8 ml/min; Solvents: A=H₂O (0.1% formic acid); B=ACN (0.1% formic acid)

### Gradient:

| (min) | % A | % B |
|---|---|---|
| 0 | 98 | 2 |
| 2.50 | 0 | 100 |
| 2.90 | 0 | 100 |
| 2.95 | 98 | 2 |
| 3.00 | 98 | 2 |

### Method K:

System Waters UPLC/SQD; ionization:electrospray in positive and/or negative mode (ES+/−); Column: ACQUITY CORTECS C18 1.6 μm 2.1×50 mm; Column temperature: 50° C.; Flow: 1.0 ml/min; Solvents: A=H₂O (0.1% trifluoroacetic acid); B=ACN (0.1% trifluoroacetic acid).

### Gradient:

| (min) | % A | % B |
|---|---|---|
| 0 | 97 | 3 |
| 0.1 | 97 | 7 |
| 2.1 | 3 | 97 |
| 2.4 | 3 | 97 |
| 2.5 | 97 | 3 |
| 3.00 | 97 | 3 |

### Method L:

System Waters UPLC/SQD; ionization:electrospray in positive and/or negative mode (ES+/−); Column: ACQUITY CORTECS C18 1.6 μm 2.1×50 mm; Column temperature: 50° C.; Flow: 1.0 ml/min; Solvents: A=H₂O (0.1% formic acid); B=ACN (0.1% formic acid)

### Gradient:

| (min) | % A | % B |
|---|---|---|
| 0 | 95 | 5 |
| 0.05 | 94 | 6 |
| 1.50 | 1 | 99 |
| 1.90 | 1 | 99 |
| 1.95 | 95 | 5 |
| 2.00 | 95 | 5 |

### Method M:

System Waters UPLC-SQD2; ionization:electrospray in positive and/or negative mode (ES+/−); Column: ACQUITY CSH C18 1.7 μm 2.1×30 mm; Column temperature: 35° C. Flow: 0.7 ml/min; Solvents: A=H₂O (0.1% formic acid); B=ACN (0.1% formic acid)

### Gradient:

| (min) | % A | % B |
|---|---|---|
| 0 | 97 | 3 |
| 0.15 | 97 | 3 |
| 3.30 | 3 | 97 |
| 3.45 | 3 | 97 |
| 3.85 | 97 | 3 |
| 4.00 | 97 | 3 |

### Method N:

System Waters UPLC/SQD; ionization:electrospray in positive and/or negative mode (ES+/−); Column: ACQUITY CORTECS C18 1.6 μm 2.1×50 mm; Column temperature: 55° C.; Flow: 0.8 ml/min; Solvents: A=H₂O (0.1% formic acid); B=ACN (0.1% formic acid)

### Gradient:

| (min) | % A | % B |
|---|---|---|
| 0 | 60 | 40 |
| 0.15 | 60 | 40 |
| 3.30 | 0 | 100 |
| 3.45 | 0 | 100 |
| 3.85 | 98 | 2 |
| 4.00 | 98 | 2 |

### Method O:

System Waters UPLC-SQD2; ionization:electrospray in positive and/or negative mode (ES+/−); Column: ACQUITY CSH C18 1.7 μm 2.1×30 mm; Column temperature: 35° C. Flow: 0.7 ml/min; Solvents: A=H₂O (0.1% formic acid); B=ACN (0.1% formic acid)

### Gradient:

| (min) | % A | % B |
|---|---|---|
| 0 | 95 | 5 |
| 0.05 | 94 | 6 |
| 1.50 | 1 | 99 |
| 1.90 | 1 | 99 |
| 1.95 | 98 | 2 |
| 2.00 | 98 | 2 |

### Method Q:

System Waters UPLC-DAD and QDa; ionization:electrospray in positive and/or negative mode (ES+/−); Column: HSS-T3 50 mm*2.1 mm-1.8 μm; Column temperature: 40° C.; Flow: 0.45 ml/min; Solvents: A=H₂O 95%, ACN 5% (0.5 g/l AcNH₄); B=CAN; C=Acide formique 0.2%

### Gradient:

| (min) | % A | % B | % C |
|---|---|---|---|
| 0 | 49 | 2 | 49 |
| 0.1 | 49 | 2 | 49 |
| 0.25 | 49 | 2 | 49 |
| 1.8 | 8 | 84 | 8 |
| 2.8 | 8 | 84 | 8 |
| 3.0 | 49 | 2 | 49 |
| 3.8 | 49 | 2 | 49 |
| 3.9 | 49 | 2 | 49 |

### Method R:

System Waters UPLC-DAD and QDa; ionization:electrospray in positive and/or negative mode (ES+/−); Column: BEH-C18 50 mm*2.1 mm-1.7 μm; Column temperature: 40° C.; Flow: 0.5 ml/min; Solvents: A=H₂O 95%, ACN 5% (0.5 g/l AcNH₄); B=ACN

### Gradient:

| (min) | % A | % B |
|---|---|---|
| 0 | 95 | 5 |
| 0.2 | 95 | 5 |
| 1.2 | 5 | 95 |
| 2.8 | 5 | 95 |
| 3.0 | 95 | 5 |
| 3.4 | 95 | 5 |

### Method S:

System Waters UPLC-DAD and QDa; ionization:electrospray in positive and/or negative mode (ES+/−); Column: ACQUITY BEH C18 1.7 μm 2.1×50 mm; Column temperature: 40° C. Flow: 0.50 ml/min; Solvents: A=H₂O 95%, ACN 5% (0.5 g/l AcNH₄); B=ACN Gradient:

| (min) | % A | % B |
|---|---|---|
| 0 | 95 | 5 |
| 1 | 5 | 95 |
| 2.6 | 5 | 95 |
| 2,8 | 95 | 5 |
| 3,3 | 95 | 5 |
| 3,5 | 5 | 95 |

### Method T:

System Waters UPLC-DAD and ZQ; ionization:electrospray in positive and/or negative mode (ES+/−); Column: Xbridge C18 100 mm*4.6 mm 5 μm; Column temperature: 30° C.; Flow: 0.8 ml/min; Solvents: A=H₂O (0.2% NH₄HCO₃); B=ACN

### Gradient:

| (min) | % A | % B |
|---|---|---|
| 0 | 80 | 20 |
| 0.5 | 80 | 20 |
| 5.0 | 10 | 90 |
| 9.0 | 10 | 90 |
| 10.5 | 80 | 20 |
| 12 | 80 | 20 |

### Method U:

System Waters UPLC-SQD2; ionization:electrospray in positive and/or negative mode (ES+/−); Column: ACQUITY CSH C18+1.7 μm 2.1×50 mm; Column temperature: 60° C.; Flow: 1.0 ml/min; Solvents: A=H₂O (0.1% formic acid); B=ACN (0.1% formic acid)

### Gradient:

| (min) | % A | % B |
|---|---|---|
| 0 | 97 | 3 |
| 0.1 | 97 | 3 |
| 2.10 | 3 | 97 |
| 2.45 | 3 | 97 |
| 3.0 | 97 | 3 |

### Method V:

System Waters UPLC-SQD2; ionization:electrospray in positive and/or negative mode (ES+/−); Column: ACQUITY CORTECS C18 1.6 μm 2.1×50 mm; Column temperature: 50° C.; Flow: 1 ml/min; Solvents: A=H₂O (0.1% formic acid); B=ACN (0.1% formic acid)

### Gradient:

| (min) | % A | % B |
|---|---|---|
| 0 | 95 | 5 |
| 1.50 | 1 | 99 |
| 1.90 | 1 | 99 |
| 1.95 | 95 | 5 |
| 2.00 | 95 | 5 |

### Method W:

System Waters UPLC-SQD2; ionization:electrospray in positive and/or negative mode (ES+/−); Column: ACQUITY CORTECS C18 1.6 μm 2.1×50 mm; Column temperature: 50° C.; Flow: 1 ml/min; Solvents: A=H₂O (0.1% formic acid); B=ACN (0.1% formic acid)

### Gradient:

| (min) | % A | % B |
|---|---|---|
| 0 | 100 | 0 |
| 0.20 | 100 | 0 |
| 1.50 | 1 | 99 |
| 1.90 | 1 | 99 |
| 1.95 | 95 | 5 |
| 2.00 | 95 | 5 |

### Method X:

System Waters UPLC/SQD; ionization:electrospray in positive and/or negative mode (ES+/−); Column: ACQUITY CSH C18 1.7 μm 2.1×50 mm; Column temperature: 50° C.; Flow: 1.0 ml/min; Solvents: A=H₂O (0.1% formic acid); B=ACN (0.1% formic acid)

### Gradient:

| (min) | % A | % B |
|---|---|---|
| 0 | 95 | 5 |
| 0.05 | 95 | 5 |
| 1.50 | 2 | 98 |
| 1.90 | 2 | 98 |
| 1.95 | 95 | 5 |
| 2.00 | 95 | 5 |

### Method Y:

System Waters UPLC/SQD; ionization:electrospray in positive and/or negative mode (ES+/−); Column: ACQUITY CSH C18 1.7 μm 2.1×50 mm; Column temperature: 50° C.; Flow: 1.0 ml/min; Solvents: A=H₂O (0.1% formic acid); B=ACN (0.1% formic acid)

### Gradient:

| (min) | % A | % B |
|---|---|---|
| 0 | 95 | 5 |
| 0.05 | 94 | 6 |
| 2.50 | 1 | 99 |
| 3.90 | 1 | 99 |
| 3.95 | 95 | 5 |
| 4.00 | 95 | 5 |

### Method Z4:

System Agilent Technologies 1200 Series; ionization:electrospray in positive and/or negative mode (ES+/−); Column: XBridge C18 3.5 μm 4.6×50 mm; Column temperature: 45° C.; Flow: 1.8 ml/min Solvents: A=H₂O (10 mM NH₄HCO₃); B=ACN

### Gradient:

| (min) | % A | % B |
|---|---|---|
| 0 | 95 | 5 |
| 1.5 | 5 | 95 |

### Method Z5:

System Agilent Technologies 1200 Series; ionisation: electrospray in positive and/or negative mode (ES+/−); Column: Poroshell 120 EC C18 4 μm 4.6×50 mm; Column temperature: 45° C.; Flow: 2.0 ml/min; Solvents: A=H₂O (0.01% TFA); B=CAN (0.010% TFA)

### Gradient:

| (min) | % A | % B |
|---|---|---|
| 0 | 95 | 5 |
| 1.5 | 5 | 95 |

### Method Z6:

System Agilent Technologies 1200 Series; ionization:electrospray in positive and/or negative mode (ES+/−); Column: Sunfire 3.5 μm 4.6×50 mm; Column temperature: 45° C.; Flow: 2.0 ml/min; Solvents: A=H₂O (0.01% TFA); B=CAN (0.01% TFA)

### Gradient:

| (min) | % A | % B |
|---|---|---|
| 0 | 95 | 5 |
| 1.3 | 5 | 95 |
| 3.0 | 5 | 95 |

Mass spectrometry results are reported as the ratio of mass over charge.

Reactions under microwaves were realized using a Biotage Initiator apparatus.

### Synthesis of Examples According to Reaction Scheme 1

### Example 8: 2-[6-[(2S)-2-(hydroxymethyl)morpholin-4-yl]pyridazin-3-yl]-3,5-dimethyl-phenol

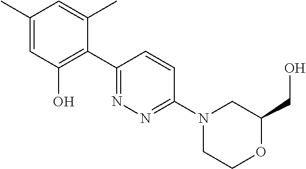 [SMILES: Cc1cc(C)c(-c2ccc(N3CCO[C@H](CO)C3)nn2)c(O)c1]

### Int 8.1: [(2S)-4-(6-chloropyridazin-3-yl)morpholin-2-yl]methanol

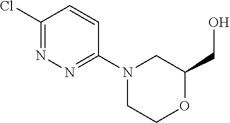 [SMILES: OC[C@@H]1CN(c2ccc(Cl)nn2)CCO1]

In a microwave vial, under Ar, to a solution of 3,6-dichloropyridazine (2.6 mmol, 400 mg) in 2.5 ml of NMP was added [(2S)-morpholin-2-yl]methanol; hydrochloride (2.6 mmol, 400 mg) and DIPEA (5.2 mmol, 0.9 ml). The vial was sealed, and the mixture was heated at 180° C. under microwave irradiation for 2.5 h. After cooling down the solution, EtOAc was added, and the solution was washed with water (3 times) and brine. The aqueous layer was extracted twice with Me-THF. The combined organic layers were dried over Na₂SO₄, filtered and concentrated under reduced pressure. The residue was purified by flash chromatography on silica gel using DCM/MeOH/ACN (from 100/0/0 to 96/2/2) to give the title compound as a yellow solid (214 mg, 36% yield).

LCMS (method G): Rt=1.19 min; MS m/z [M+H]⁺ 230

### 2-[6-[(2S)-2-(hydroxymethyl)morpholin-4-yl]pyridazin-3-yl]-3,5-dimethyl-phenol (Example 8)

In a microwave vial, under Ar, to a solution of (2-hydroxy-4,6-dimethyl-phenyl)boronic acid (0.72 mmol, 120 mg) and [(2S)-4-(6-chloropyridazin-3-yl)morpholin-2-yl]methanol (0.79 mmol, 183 mg) in 4.5 ml of 1,4-dioxane and 1.2 ml of water was added sodium carbonate (1.59 mmol, 132 mg). The solution was purged with Ar for 10 min, then Pd(PPh₃)₄ (0.036 mmol, 42 mg) was added. The vial was sealed, and the mixture was heated at 110° C. under microwave irradiation for 2.5 h. After concentration under reduced pressure, EtOAc was added, and the resulting mixture was washed with water (3 times) and brine. The organic layer was dried over Na₂SO₄, filtered and concentrated under reduced pressure. The residue was purified by flash chromatography on silica gel using DCM/MeOH/ACN (from 98/1/1 to 94/2/2) to give the title compound as a white solid (92 mg, 40% yield).

Compounds Examples 9, 65 and 179 were synthesized analogously to Example 8.

Compounds Examples 14, 15, 16, and 20 were synthesized analogously to Example 8 using Pinacolborane instead of boronic acid for Suzuki coupling.

Compounds Examples 24 and 30 were synthesized analogously to Example 8 using Pinacolborane instead of boronic acid for Suzuki coupling and using K₂CO₃/DMF instead of DIPEA/NMP at 110° C. for n-arylation.

Compound Example 79 was synthesized analogously to Example 8 for Suzuki coupling and to Example 85 for n-arylation.

Compound Example 83 was synthesized analogously to Example 8 using BuOH instead of NMP at 130° C. for n-arylation.

Compounds Examples 84 and 91 were synthesized analogously to Example 8 using DME instead of 1,4-dioxane for Suzuki coupling and using BuOH instead of NMP at 130° C. for n-arylation.

Compound Example 113 was synthesized analogously to Example 8 using DMSO instead of NMP at 150° C. for n-arylation.

Compound Example 418 was synthesized analogously to Example 8.

### Examples 10 and 11: 2-[6-[(4aR,8aS)-6-cyclobutyl-3,4a,5,7,8,8a-hexahydro-2H-pyrido[4,3-b][1,4]oxazin-4-yl]pyridazin-3-yl]-3,5-dimethyl-phenol and 2-[6-[(4aS,8aR)-6-cyclobutyl-3,4a,5,7,8,8a-hexahydro-2H-pyrido[4,3-b][1,4]oxazin-4-yl]pyridazin-3-yl]-3,5-dimethyl-phenol

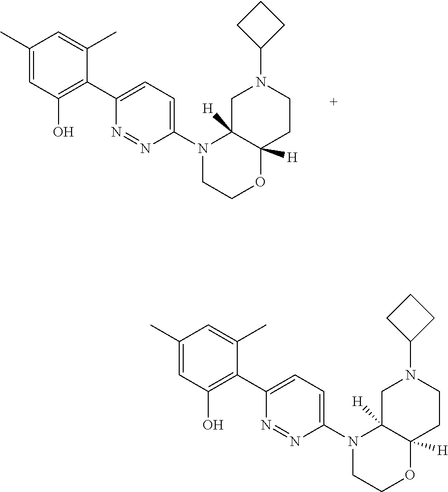 [SMILES: Cc1cc(C)c(-c2ccc(N3CCO[C@H]4CCN(C5CCC5)C[C@H]43)nn2)c(O)c1 ; Cc1cc(C)c(-c2ccc(N3CCO[C@@H]4CCN(C5CCC5)C[C@@H]43)nn2)c(O)c1]

### Int 10.7: benzyl rac-(4aR,8aS)-2,3,4,4a,5,7,8,8a-octahydropyrido[4,3-b][1,4]oxazine-6-carboxylate

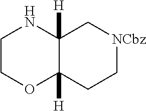 [SMILES: O=C(OCc1ccccc1)N1CC[C@@H]2OCCN[C@@H]2C1]

Under N₂, at 0° C., to a solution of benzyl rac-(4aR,8aS)-3-oxo-4,4a,5,7,8,8a-hexahydropyrido[4,3-b][1,4]oxazine-6-carboxylate (9.47 mmol, 2.75 g) in 25 ml of THF was added a 1.0 M solution of BH₃-THF (8.25 mmol, 8.24 ml). The solution was stirred at 60° C. for 8 h. Then at 0° C., 4 ml of MeOH are added and the mixture was stirred at 60° C. for another 1 h. 25 ml of an aqueous solution of 1M HCl was added, and the mixture was stirred at 40° C. for additional 0.5 h. The reaction mixture was concentrated under reduced pressure to give crude benzyl (4aR,8aS)-hexahydro-2H-pyrido[4,3-b][1,4]oxazine-6(5H)-carboxylate (3 g) as a yellow oil. To this oil was added in 30 ml of DCM, then Et₃N (2.3 ml), Boc₂O (16 mmol, 3.55 g) and DMAP (133 mg). The resulting solution was stirred for 6 h at 25° C. The reaction as then poured into 30 ml of water and extracted with DCM (3 times). The combined organic layers are dried over Na₂SO₄, filtered and concentrated under reduced pressure. The residue was purified by flash chromatography on silica gel using Petroleum ether/Ethyl acetate (from 10/1 to 1/1) to give 4.1 g of 6-benzyl 4-(tert-butyl) (4aR,8aS)-hexahydro-4H-pyrido[4,3-b][1,4]oxazine-4,6(5H)-dicarboxylate as a colourless oil. To 1.25 g of this oil was added 5 ml of 4M HCl/EtOAc. The solution was stirred at 25° C. for 1 h, then concentrated under reduced pressure to give 800 mg of crude benzyl (4aR,8aS)-hexahydro-2H-pyrido[4,3-b][1,4]oxazine-6(5H)-carboxylate as a yellow oil, which was used for next step without further purification.

### Int 10.8: benzyl rac-(4aR,8aS)-4-(6-chloropyridazin-3-yl)-3,4a,5,7,8,8a-hexahydro-2H-pyrido[4,3-b][1,4]oxazine-6-carboxylate

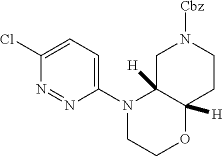 [SMILES: O=C(OCc1ccccc1)N1CC[C@@H]2OCCN(c3ccc(Cl)nn3)[C@@H]2C1]

To a solution of crude benzyl rac-(4aR,8aS)-2,3,4,4a,5,7,8,8a-octahydropyrido[4,3-b][1,4]oxazine-6-carboxylate (2.06 mmol, 800 mg, chlorhydrate) in 5 ml of NMP was added 1.5 ml of DIPEA and 3,6-dichloropyridazine (10 mmol, 2.16 g) and the resulting mixture was stirred at 180° C. under microwave irradiation for 1 h. After cooling down the solution, the reaction was quenched with water and extracted with EtOAc (3 times). The combined organic layers were washed with brine, dried over Na₂SO₄, filtered and concentrated under reduced pressure. The residue was purified by prep-HPLC (column: Phenomenex luna C18 150*40 mm*15 μm; mobile phase: [water (HCOOH)-ACN]; B %: 42%-72%, 10 min) to give the title compound as a yellow solid (450 mg, 40% yield).

¹H NMR (400 MHz, CDCl₃) δ=7.38 (br s, 5H), 7.14-6.85 (m, 1H), 6.78-6.57 (m, 1H), 5.35-4.93 (m, 2H), 4.26-3.63 (m, 7H), 3.34-2.96 (m, 3H), 2.04-1.79 (m, 2H).

### Int 10.9: (2-hydroxy-4,6-dimethylphenyl)boronic acid

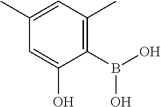

At −78° C., to a solution of (2-methoxy-4,6-dimethyl-phenyl)boronic acid (555 μmol, 100 mg) in 2 ml of DCM was added, BBr₃ (1.1 mmol, 107 μl) and the mixture was stirred at −78° C. for 1 h. The reaction was then quenched by addition of 10 ml of ice water at 0° C., and extracted with DCM (3 times). The combined organic layers were washed with brine, dried over Na₂SO₄, filtered and concentrated under reduced pressure. The residue was purified by recrystallization from Petroleum ether to give the title compound as a yellow solid (40 mg, 40% yield).

¹H NMR (400 MHz, DMSO-d₆) δ=9.12-8.86 (m, 1H), 6.70 (s, 1H), 6.61 (s, 1H), 2.46 (s, 3H), 2.25 (s, 3H).

### Int 10.10: benzyl rac-(4aR,8aS)-4-[6-(2-hydroxy-4,6-dimethyl-phenyl)pyridazin-3-yl]-3,4a,5,7,8,8a-hexahydro-2H-pyrido[4,3-b][1,4]oxazine-6-carboxylate

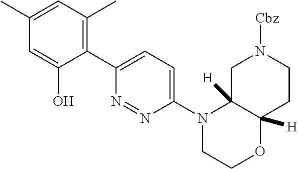 [SMILES: Cc1cc(C)c(-c2ccc(N3CCO[C@H]4CCN(C(=O)OCc5ccccc5)C[C@H]43)nn2)c(O)c1]

A mixture of benzyl rac-(4aR,8aS)-4-(6-chloropyridazin-3-yl)-3,4a,5,7,8,8a-hexahydro-2H-pyrido[4,3-b][1,4]oxazine-6-carboxylate (0.85 mmol, 330 mg), (2-methoxy-4,6-dimethyl-phenyl)boronic acid (1.70 mmol, 282 mg), Pd(PPh₃)₄ (98 mg), 2 M aqueous solution of Na₂CO₃ (1.27 ml) and 4 ml of 1,4-dioxane was degassed and purged with N₂ for 3 times, and then the mixture was stirred at 140° C. for 2 h under microwave irradiation. The reaction mixture was then filtered, and the filtrate was concentrated under reduced pressure. The residue was purified by flash chromatography on silica gel using Petroleum ether/Ethyl acetate (from 20/1 to 5/1) to give the title compound as a yellow oil (320 mg, 82% yield). ¹H NMR (400 MHz, CDCl₃) δ=7.65-7.46 (m, 1H), 7.38 (br s, 5H), 6.86-6.71 (m, 2H), 6.66 (s, 1H), 5.38-4.91 (m, 2H), 4.25-3.89 (m, 6H), 3.79 (dt, J=3.2, 11.6 Hz, 1H), 3.48-2.99 (m, 3H), 2.45-2.25 (m, 6H), 2.02-1.85 (m, 2H).

### Int 10.11 or Example 195: 3,5-dimethyl-2-[6-[rac-(4aR,8aS)-2,3,4a,5,6,7,8,8a-octahydropyrido[4,3-b][1,4]oxazin-4-yl]pyridazin-3-yl]phenol

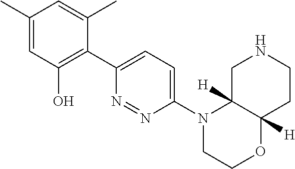 [SMILES: Cc1cc(C)c(-c2ccc(N3CCO[C@H]4CCNC[C@H]43)nn2)c(O)c1]

To a solution of benzyl rac-(4aS,8aR)-1-[6-(2-hydroxy-4,6-dimethyl-phenyl)pyridazin-3-yl]-3,4a,5,7,8,8a-hexahydro-2H-pyrido[3,4-b][1,4]oxazine-6-carboxylate (2.3 mmol, 1.1 g) in 25 ml of EtOH was added ammonium formate (7 mmol, 440 mg) and 10% Pd/C (440 mg) and the resulting mixture was stirred at 80° C. under microwave irradiation for 1 h 30. After cooling down to RT, the mixture was filtered off over a pad of Decalite® and the cake rinsed with 50 ml of EtOH. The organic layer was concentrated under reduced pressure and the residue was diluted with 200 ml of dichloromethane and washed twice with 200 ml of an aqueous saturated sodium bicarbonate solution, dried over Na₂SO₄, filtered and concentrated under reduced pressure. The residue was purified by flash chromatography on silica gel using a gradient of DCM/MeOH/ACN (from 100/0/0 to 80/10/10) to give the title compound as a white solid (351 mg, 44% yield).

### 2-[6-[(4aR,8aS)-6-cyclobutyl-3,4a,5,7,8,8a-hexahydro-2H-pyrido[4,3-b][1,4]oxazin-4-yl]pyridazin-3-yl]-3,5-dimethyl-phenol (Example 10) and 2-[6-[(4aS,8aR)-6-cyclobutyl-3,4a,5,7,8,8a-hexahydro-2H-pyrido[4,3-b][1,4]oxazin-4-yl]pyridazin-3-yl]-3,5-dimethyl-phenol (Example 11)

To a solution of 3,5-dimethyl-2-[6-[rac-(4aR,8aS)-2,3,4a,5,6,7,8,8a-octahydropyrido[4,3-b][1,4]oxazin-4-yl]pyridazin-3-yl]phenol (0.53 mmol, 180 mg) in 3 ml of DCM was added cyclobutanone (1 mmol, 37 mg), AcOK (1 mmol, 52 mg) and NaBH₃CN (2.6 mmol, 166 mg) and the mixture as stirred at 25° C. for 8 h. The reaction was then concentrated under reduced pressure and the residue was purified by prep-HPLC (column: Phenomenex Luna C18 150*25 mm*10 μm; mobile phase: [water (HCOOH)-ACN]; B %: 29%-59%, 10 min) and lyophilized. Then, this racemate was separated by SFC (Column: Daicel Chiralpak® AD, 250×30 mm I.D., 10 μm particle size; Mobile phase: Eluent B: EtOH (0.1% NH₄OH), Isocratic elution: 40% Phase B in Supercritical C₀₂; Flow rate: 70 g/min; Cycle time: 3.5 min; Back Pressure: 100 bar) to provide the 2 enantiomers, first one eluting at Rt=1.99 min (60.48 mg, Example 10, Enantiomer 1) as a white solid, and second one eluting at Rt=2.26 min (64.8 mg, Example 11, Enantiomer 2) as a white solid. (The absolute configurations cannot be assigned).

Compounds Examples 12 and 13 were synthesized analogously to Examples 10 and 11 using Pinacol borolane/Na₂CO₃ instead of boronic acid/NaHCO₃ for Suzuki coupling.

### Example 4: 3,5-dimethyl-2-[6-[rac-(4aS,8aR)-6-cyclobutyl-3,4a,5,7,8,8a-hexahydro-2H-pyrido[3,4-b][1,4]oxazin-1-yl]pyridazin-3-yl]phenol

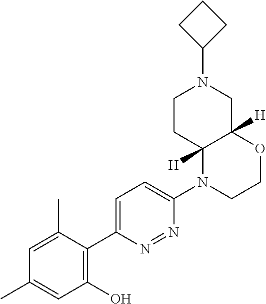 [SMILES: Cc1cc(C)c(-c2ccc(N3CCO[C@H]4CN(C5CCC5)CC[C@H]43)nn2)c(O)c1]

### Int 4.10: benzyl rac-(4aS,8aR)-1-(6-chloropyridazin-3-yl)-3,4a,5,7,8,8a-hexahydro-2H-pyrido[3,4-b][1,4]oxazine-6-carboxylate

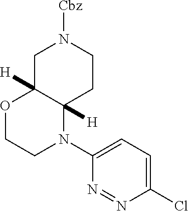 [SMILES: O=C(OCc1ccccc1)N1CC[C@H]2[C@@H](C1)OCCN2c1ccc(Cl)nn1]

To a solution of 3,6-dichloropyridazine (8.2 mmol, 1.2 g) in 4 ml of NMP was added DIPEA (851 μl, 65 mmol) and benzyl rac-(4aS,8aR)-1,2,3,4a,5,7,8,8a-octahydropyrido[3,4-b][1,4]oxazine-6-carboxylate (1.63 mmol, 450 mg) and the mixture was stirred at 180° C. for 1 h under microwave irradiation. After cooling down the solution, water was added, and the mixture was extracted with EtOAc (3 times). The combined organic layers were washed with brine, dried over Na₂SO₄, filtered and concentrated under reduced pressure. The residue was purified by prep-HPLC (column: Phenomenex luna C18 150*40 mm*15 μm; mobile phase: [water (HCOOH)-ACN]; B %: 36%-66%, 9 min) to give the title compound as a yellow solid (270 mg, 42% yield).

¹H NMR (400 MHz, CDCl₃) δ=7.41-7.28 (m, 5H), 7.24 (d, J=9.6 Hz, 1H), 6.83 (d, J=9.6 Hz, 1H), 5.16 (br d, J=17.2 Hz, 2H), 4.62-4.20 (m, 3H), 4.16-4.07 (m, 1H), 3.73 (dt, J=3.2, 12.0 Hz, 2H), 3.65-3.53 (m, 1H), 3.27 (dt, J=3.6, 12.2 Hz, 1H), 3.15-2.75 (m, 2H), 2.19-1.97 (m, 1H), 1.63 (br s, 1H).

### Int 4.11: benzyl rac-(4aS,8aR)-1-[6-(2-hydroxy-4,6-dimethyl-phenyl)pyridazin-3-yl]-3,4a,5,7,8,8a-hexahydro-2H-pyrido[3,4-b][1,4]oxazine-6-carboxylate

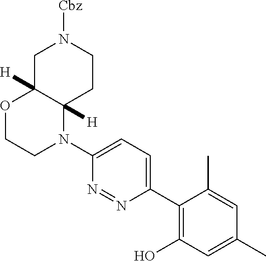 [SMILES: Cc1cc(C)c(-c2ccc(N3CCO[C@@H]4CN(C(=O)OCc5ccccc5)CC[C@@H]43)nn2)c(O)c1]

A mixture of benzyl rac-(4aS,8aR)-1-(6-chloropyridazin-3-yl)-3,4a,5,7,8,8a-hexahydro-2H-pyrido[3,4-b][1,4]oxazine-6-carboxylate (103 μmol, 40 mg), (2-hydroxy-4,6-dimethyl-phenyl) boronic acid (103 μmol, 17 mg), 2M aqueous solution of Na₂CO₃ (155 μl), Pd(PPh₃)₄ (12 mg) in 1.5 ml of 1,4-dioxane was degassed and purged with N₂ (3 times), and the mixture was stirred at 140° C. for 2 h under microwave irradiation. Then, the reaction was filtered, and the filtrate concentrated under reduced pressure. The residue was purified by prep-HPLC (column: Phenomenex Luna C18 150*25 mm*10 μm; mobile phase: [water (HCOOH)-ACN]; B %: 29%-59%, 10 min) to give the title compound as a yellow solid (26 mg, 53% yield).

¹H NMR (400 MHz, CDCl₃) δ=7.54 (d, J=9.6 Hz, 1H), 7.43-7.30 (m, 5H), 6.98 (d, J=9.6 Hz, 1H), 6.77 (s, 1H), 6.66 (s, 1H), 5.26-5.10 (m, 2H), 4.70-4.56 (m, 1H), 4.21-4.09 (m, 1H), 3.83-3.63 (m, 3H), 3.45-3.28 (m, 1H), 3.16-2.82 (m, 2H), 2.39 (s, 3H), 2.31 (s, 3H), 2.24-2.11 (m, 2H), 1.75-1.64 (m, 2H).

### Int 4.12: 3,5-dimethyl-2-[6-[rac-(4aS,8aR)-2,3,4a,5,6,7,8,8a-octahydropyrido[3,4-b][1,4]oxazin-1-yl]pyridazin-3-yl]phenol

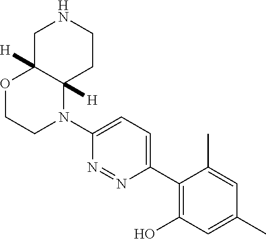 [SMILES: Cc1cc(C)c(-c2ccc(N3CCO[C@@H]4CNCC[C@@H]43)nn2)c(O)c1]

Under N₂, to a solution of benzyl rac-(4aS,8aR)-1-[6-(2-hydroxy-4,6-dimethyl-phenyl)pyridazin-3-yl]-3,4a,5,7,8,8a-hexahydro-2H-pyrido[3,4-b][1,4]oxazine-6-carboxylate (0,055 mmol, 26 mg) in 4 ml of EtOAc was added Pd/C (7 mg, 10% purity). The suspension was degassed, purged with H₂ (3 times), and the mixture was stirred under H₂ (15 Psi) at 25° C. for 2 h. The reaction was then filtered, and the filtrate was concentrated under reduced pressure to give 18 mg of the crude title compound as a colorless oil, which was used for next step without further purification.

### 3,5-dimethyl-2-[6-[rac-(4aS,8aR)-6-cyclobutyl-3,4a,5,7,8,8a-hexahydro-2H-pyrido [3,4-b][1,4]oxazin-1-yl]pyridazin-3-yl]phenol (Example 4)

To a solution of 3,5-dimethyl-2-[6-[rac-(4aS,8aR)-2,3,4a,5,6,7,8,8a-octahydropyrido[3,4-b][1,4]oxazin-1-yl]pyridazin-3-yl]phenol (0.053 mmol, 18 mg) in 1 ml of DCM was added cyclobutanone (0.058 mmol, 4.35 μl), AcOK (0.058 mmol) and NaBH₃CN (0.105 mmol, 6.6 mg). The mixture was stirred at rt for 4 h. The reaction was then concentrated under reduced pressure, and the residue was purified by prep-HPLC (column: Phenomenex luna C18 150*25 mm*10 μm; mobile phase: [water (HCOOH)-ACN]; B %: 1%-30%, 10 min) to give the title compound as a white solid (8 mg, 37% yield).

### Example 6: 2-[6-[(3S)-3-(hydroxymethyl)morpholin-4-yl]pyridazin-3-yl]-3,5-dimethyl-phenol

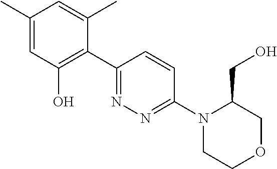 [SMILES: Cc1cc(C)c(-c2ccc(N3CCOC[C@@H]3CO)nn2)c(O)c1]

### Int 6.1: [(3S)-4-(6-chloropyridazin-3-yl)morpholin-3-yl]methanol

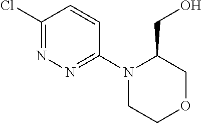 [SMILES: OC[C@H]1COCCN1c1ccc(Cl)nn1]

In a microwave vial, under Ar, to a solution of 3,6-dichloropyridazine (5.2 mmol, 800 mg) in 3.5 ml of NMP was added [(3S)-morpholin-3-yl]methanol; hydrochloride (5.2 mmol, 800 mg) and DIPEA (10.4 mmol, 1.83 ml). The vial was sealed, and the mixture was heated at 180° C. under microwave irradiation for 2.5 h. After cooling down the solution, EtOAc was added, and the solution was washed with water (3 times) and brine. The aqueous layer was extracted twice with Me-THF. The combined organic layers were dried over Na₂SO₄, filtered and concentrated under reduced pressure. The residue was purified by flash chromatography on silica gel using DCM/MeOH/ACN (from 100/0/0 to 96/2/2) to give the title compound as an orange oil (460 mg, 38% yield).

LCMS (Method G): Rt=1.17 min; MS m/z [M+H]⁺ 230

### 2-[6-[(3S)-3-(hydroxymethyl)morpholin-4-yl]pyridazin-3-yl]-3,5-dimethyl-phenol (Example 6)

In a microwave vial, under Ar, to a solution of (2-hydroxy-4,6-dimethyl-phenyl)boronic acid (0.58 mmol, 97 mg) and [(3S)-4-(6-chloropyridazin-3-yl)morpholin-3-yl]methanol (0.96 mmol, 221 mg) in 1,4-dioxane (3.64 ml) was added a solution of sodium carbonate (1.28 mmol, 106.7 mg) in water (0.91 ml). The solution was purged with Ar for 10 min, then Pd(PPh₃)₄ (0.029 mmol, 34 mg) was added. The vial was sealed, and the mixture was heated at 75° C. under microwave irradiation for 1 h, and 110° C. for 2 h. Then (2-hydroxy-4,6-dimethyl-phenyl)boronic acid (0.15 mmol, 25 mg) was added, and the mixture was heated at 100° C. under microwave irradiation for 4 h. After concentration under reduced pressure, EtOAc was added, and the resulting mixture was washed with water (3 times) and brine. The organic layer was dried over Na₂SO₄, filtered and concentrated under reduced pressure. The residue was purified by flash chromatography on silica gel using DCM/MeOH/ACN (from 98/1/1 to 94/3/3) to give the title compound as a yellow solid (52 mg, 28% yield).

Compound Example 5 was synthesized analogously to Example 6.

### Example 62: (4S)-4-hydroxy-1-[6-[2-hydroxy-6-methyl-4-(trifluoromethyl)phenyl]pyridazin-3-yl]pyrrolidin-2-one

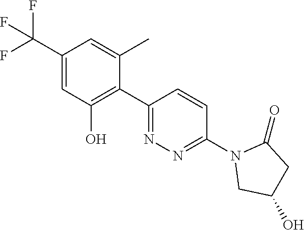 [SMILES: Cc1cc(C(F)(F)F)cc(O)c1-c1ccc(N2C[C@@H](O)CC2=O)nn1]

### Int 62.1: (4S)-1-(6-chloropyridazin-3-yl)-4-hydroxy-pyrrolidin-2-one

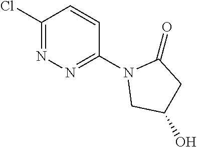 [SMILES: O=C1C[C@H](O)CN1c1ccc(Cl)nn1]

In a microwave vial, under Ar, to a solution of 3,6-dichloropyridazine (2.25 mmol, 335 mg) and (S)-4-hydroxy-2-pyrrolidinone (2.25 mmol, 227 mg) in 5 ml of 1,4-dioxane was added potassium phosphate tribasic (4.5 mmol, 955 mg), 1-1′-bis(diphenylphosphino)ferrocene (0.27 mmol, 150 mg), palladium acetate (0.090 mmol, 20 mg). The vial was sealed, and the mixture was heated at 100° C. under microwave irradiation for 1 h. After cooling down to rt, the reaction mixture was filtered, the solid part was washed with EtOAc and the combined filtrate was concentrated under reduced pressure. The residue was purified by flash chromatography on silica gel using DCM/EtOAc (from 100/0 to 20/80) to give the title compound as a brown solid (74 mg, 15% yield).

LCMS (Method M): Rt=0.76 min; MS m/z [M+H]⁺ 214

### (4S)-4-hydroxy-1-[6-[2-hydroxy-6-methyl-4-(trifluoromethyl)phenyl]pyridazin-3-yl]pyrrolidin-2-one (Example 62)

In a microwave vial, under Ar, to a solution of 3-methyl-2-(4,4,5,5-tetramethyl-1,3,2-dioxaborolan-2-yl)-5-(trifluoromethyl)phenol (0.34 mmol, 104 mg) and (4S)-1-(6-chloropyridazin-3-yl)-4-hydroxy-pyrrolidin-2-one (0.34 mmol, 74 mg) in 1.8 ml of 1,2-dimethoxyethane are added sodium carbonate (0.86 mmol, 91 mg) and 0.5 ml of water. The solution was purged with Ar for 3 min, then Pd(PPh₃)₄ (0.02 mmol, 20 mg) was added. The vial was sealed, and the mixture was heated at 100° C. under microwave irradiation for 1 h. The reaction mixture was diluted with EtOAc and water and then filtered. The organic layer was washed with water, dried over Na₂SO₄, filtered and concentrated under reduced pressure. The residue was purified by flash chromatography on silica gel using DCM/MeOH/ACN (from 100/0/0 to 94/3/3) to give the title compound as a white solid (80 mg, 66% yield).

Compounds Examples 61 and 86 were synthesized analogously to Example 62.

### Example 63: 5-chloro-2-[6-[(2S)-2-(hydroxymethyl)morpholin-4-yl]pyridazin-3-yl]-3-methyl-phenol

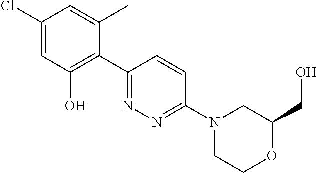 [SMILES: Cc1cc(Cl)cc(O)c1-c1ccc(N2CCO[C@H](CO)C2)nn1]

### Int 63.4: [(2S)-4-[6-(4-chloro-2-methoxy-6-methyl-phenyl)pyridazin-3-yl]morpholin-2-yl]methanol

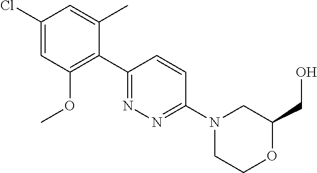 [SMILES: COc1cc(Cl)cc(C)c1-c1ccc(N2CCO[C@H](CO)C2)nn1]

Under Ar, to a solution of 2-(4-chloro-2-methoxy-6-methyl-phenyl)-4,4,5,5-tetramethyl-1,3,2-dioxaborolane (0.708 mmol, 200 mg) and [(2S)-4-(6-chloropyridazin-3-yl)morpholin-2-yl]methanol (0.708 mmol, 163 mg) in 4 ml of dimethoxyethane and 1 ml of water was added a 2N sodium carbonate aqueous solution (1.77 mmol, 0.88 ml) then tetrakis(triphenylphosphine)palladium(0) (0.035 mmol, 41 mg). The mixture was stirred at 90° C. for 3 h. After cooling down to rt, DCM was added, and the mixture was washed with water. The aqueous layer was extracted with DCM and the combined organic layers were dried over Na₂SO₄, filtered and concentrated under reduced pressure. The residue was purified by flash chromatography on silica gel eluting with a gradient of 0 to 100% of EtOAc in cyclohexane to give the title compound as a yellow solid (150 mg, 61% yield).

LCMS (Method I): Rt=1.31 min; MS m/z [M+H]⁺ 350.

### 5-chloro-2-[6-[(2S)-2-(hydroxymethyl)morpholin-4-yl]pyridazin-3-yl]-3-methyl-phenol (Example 63)

Under Ar, at 0° C., to a solution of [(2S)-4-[6-(4-chloro-2-methoxy-6-methyl-phenyl)pyridazin-3-yl]morpholin-2-yl]methanol (0.391 mmol, 137 mg) in 20 ml of anhydrous DCM was slowly added a 1N solution of boron tribromide in DCM (1.18 mmol, 1.18 ml) and the mixture was stirred at 0° C. for 3 h. then 3 ml of a 4N aqueous NaOH solution were added under vigorous stirring. After 5 min, ACN and 4N aqueous HCl solution are added, and the mixture was extracted with DCM (twice). The combined organic layers were dried over Na₂SO₄, filtered and concentrated under reduced pressure. The residue was purified by flash chromatography on silica gel eluting with a gradient of 0 to 100% of a 95/5 mixture EtOAc/MeOH in cyclohexane and the pure fractions are pooled and concentrated under reduced pressure, then triturated in diethylether to give the title compound as a light yellow solid (55 mg, 42% yield).

Compounds Example 56 was synthesized analogously to Example 63.

Compounds Examples 44, 45, 48, and 54 were synthesized analogously to Example 63 using OH-phenyl instead of OMe-phenyl and 1,4-dioxane instead of DME for Suzuki coupling. Compounds Examples 70 and 71 were synthesized analogously to Example 63 using OH-phenyl instead of OMe-phenyl and 1,4-dioxane instead of DME for Suzuki coupling and 1,4-dioxane instead of NMP during 18 h for n-arylation.

### Example 85: 5-chloro-2-[6-[(2S)-2-(hydroxymethyl)morpholin-4-yl]-4-methyl-pyridazin-3-yl]phenol

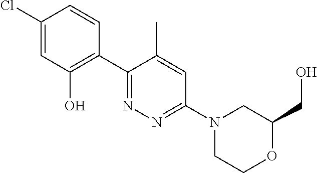 [SMILES: Cc1cc(N2CCO[C@H](CO)C2)nnc1-c1ccc(Cl)cc1O]

### Int 85.1: [(2S)-4-(6-chloro-5-methyl-pyridazin-3-yl)morpholin-2-yl]methanol

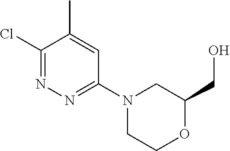 [SMILES: Cc1cc(N2CCO[C@H](CO)C2)nnc1Cl]

A solution of 3,6-dichloro-4-methyl-pyridazine (24.5 mmol, 4 g), [(2S)-morpholin-2-yl]methanol; hydrochloride (24.5 mmol, 3.77 g) and Et₃N (54 mmol, 7.52 ml) in 50 ml of n-butanol was stirred at 130° C. for 11 h. The mixture was cooled down, diluted with 100 ml of EtOAc and 50 ml of diethylether and filtered. The filtrate was concentrated under reduced pressure and the residue was purified by flash chromatography on silica gel eluting with a gradient of 0 to 100% of a 99/1 mixture EtOAc/MeOH in cyclohexane to give compound Int 85.1 (2.75 g, 46% yield) as a white solid.

LCMS (Method L): Rt=0.40 min; MS m/z [M+H]⁺ 244;

### 5-chloro-2-[6-[(2S)-2-(hydroxymethyl)morpholin-4-yl]-4-methyl-pyridazin-3-yl]phenol (Example 85)

Under Ar, to a solution of 5-chloro-2-(4,4,5,5-tetramethyl-1,3,2-dioxaborolan-2-yl)phenol (0.923 mmol, 235 mg) and [(2S)-4-(6-chloro-5-methyl-pyridazin-3-yl)morpholin-2-yl]methanol (0.615 mmol, 150 mg) in 4 ml of 1,4-dioxane was added a 2N sodium carbonate aqueous solution (1.66 mmol, 0.83 ml) then tetrakis(triphenylphosphine)palladium(0) (0.03 mmol, 35 mg). The mixture was stirred at 100° C. for 3 h. After cooling down, EtOAc and diethylether are added and the resulting mixture was washed successively with aqueous saturated NaHCO₃ and water. The organic layer was dried over Na₂SO₄, filtered and concentrated under reduced pressure. The residue was purified by flash chromatography on silica gel eluting with a gradient of 0 to 10% of MeOH in DCM to give a yellow solid residue which was purified by prep-HPLC (column: Sunfire prep C18 OBD; 250*50 mm*10 μm; mobile phase: [water (TFA 10⁻² N)-ACN]; B %: 10%-100% in 25 min. at 50 ml/min) to give the title compound as a white solid (154 mg, 74% yield).

Compounds Examples 72 to 78, 80, 81, 90, 93, 96, 97, 102, and 103 were synthesized analogously to Example 85.

Compounds Examples 109, 110, and 112 were synthesized analogously to Example 85 using OH-phenylboronic acid instead of OH-phenyl borolane for Suzuki coupling.

Compound Example 92 was synthesized analogously to Example 85 using OH-phenylboronic acid instead of OH-phenyl borolane for Suzuki coupling and using NMP instead of BuOH for n-arylation.

### Example 68: 2-[6-[(3S)-3-(ethylamino)-1-piperidyl]pyridazin-3-yl]-3,5-dimethyl-phenol

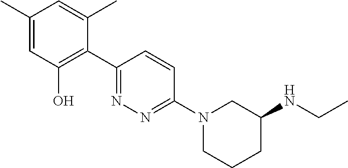 [SMILES: CCN[C@H]1CCCN(c2ccc(-c3c(C)cc(C)cc3O)nn2)C1]

### Int 68.1: tert-butyl N-[(3S)-1-(6-chloropyridazin-3-yl)-3-piperidyl]carbamate

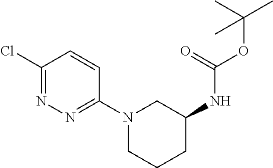 [SMILES: CC(C)(C)OC(=O)N[C@H]1CCCN(c2ccc(Cl)nn2)C1]

A solution of 3,6-dichloropyridazine (6.71 mmol, 1 g), tert-butyl N-[(3S)-3-piperidyl]carbamate (7.38 mmol, 1.48 g) and Et₃N (13.42 mmol, 1.87 ml) in 15 ml of NMP was stirred at 130° C. for 4.5 h. The mixture was cooled down, diluted with 50 ml of EtOAc and 50 ml of diethylether and washed with water (3 times). The organic layer was dried over Na₂SO₄, filtered and concentrated under reduced pressure. The residue was purified by flash chromatography on silica gel eluting with a gradient of 0 to 100% of a 95/5 mixture EtOAc/MeOH in cyclohexane to give the title compound (1.92 g, 91% yield) as a white solid. LCMS (Method I): Rt=1.67 min; MS m/z [M+H]⁺ 313

### Int 68.2: tert-butyl N-[(3S)-1-(6-chloropyridazin-3-yl)-3-piperidyl]-N-ethyl-carbamate

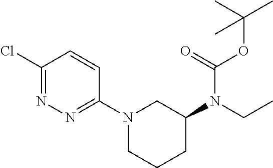 [SMILES: CCN(C(=O)OC(C)(C)C)[C@H]1CCCN(c2ccc(Cl)nn2)C1]

Under Ar, at −5° C., to a solution of tert-butyl N-[(3S)-1-(6-chloropyridazin-3-yl)-3-piperidyl]carbamate (1.6 mmol, 500 mg) in 10 ml of anhydrous DMF was added NaH (1.92 mmol, 299 mg with 60% purity) and the mixture was stirred at 0° C. for 30 min. Then, iodoethane (1.92 mmol, 153 μl) was added and the mixture was stirred at rt for 3 h. The mixture was diluted with diethylether and washed successively with an aqueous 2% citric acid solution and water (twice). The organic layer was dried over Na₂SO₄, filtered and concentrated under reduced pressure. The residue was triturated in a solution of diethylether and pentane (1/1) to give the title compound (373 mg, 68% yield) as a white solid.

LCMS (Method I): Rt=1.99 min; MS m/z [M+H]⁺ 341

### Int 68.3: tert-butyl N-ethyl-N-[(3S)-1-[6-(2-hydroxy-4,6-dimethyl-phenyl)pyridazin-3-yl]-3-piperidyl]carbamate

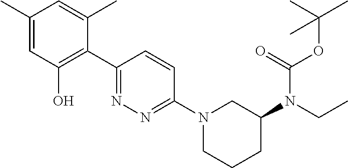 [SMILES: CCN(C(=O)OC(C)(C)C)[C@H]1CCCN(c2ccc(-c3c(C)cc(C)cc3O)nn2)C1]

Under Ar, to a solution of tert-butyl N-[(3S)-1-(6-chloropyridazin-3-yl)-3-piperidyl]-N-ethyl-carbamate (1.09 mmol, 373 mg) and (2-hydroxy-4,6-dimethyl-phenyl)boronic acid (1.42 mmol, 236 mg) in 8 ml of 1,4-dioxane and 3 ml of water was added 2N sodium carbonate aqueous solution (3.28 mmol, 1.64 ml) then tetrakis(triphenylphosphine)palladium(0) (0.08 mmol, 89 mg). The mixture was stirred at 100° C. for 13 h. After cooling down to rt, the mixture was diluted with EtOAc and diethylether, then washed successively with an aqueous 2% citric acid solution and water. The organic layer was dried over Na₂SO₄, filtered and concentrated under reduced pressure. The residue was purified by flash chromatography on silica gel eluting with a gradient of 0 to 100% of EtOAc in cyclohexane to give the title compound (350 mg, 75% yield) as a white solid.

LCMS (Method I): Rt=1.73 min; MS m/z [M+H]⁺ 427

### 2-[6-[(3S)-3-(ethylamino)-1-piperidyl]pyridazin-3-yl]-3,5-dimethyl-phenol (Example 68)

A solution of tert-butyl N-ethyl-N-[(3S)-1-[6-(2-hydroxy-4,6-dimethyl-phenyl)pyridazin-3-yl]-3-piperidyl]carbamate (0.82 mmol, 350 mg) in 18 ml of DCM and 2 ml of TFA was stirred at rt for 2 h, then concentrated under reduced pressure. The residue was dissolved in DCM and washed successively with saturated NaHCO₃ aqueous solution (twice) and water. The organic layer was dried over Na₂SO₄, filtered and concentrated under reduced pressure. The obtained solid was dried under high vacuum in the presence of phosphorus pentoxide to give the title compound as a white solid (245 mg, 91% yield).

Compound Example 69 was synthesized analogously to Example 68 using OH-phenyl boronic acid instead of OMe-phenyl Pinacolborane for Suzuki coupling and without alkylation step.

Compound 148 was synthesized analogously to Example 68.

### Examples 98 and 99: 2 diastereoisomers of 2-[6-[2-[(1S)-1-hydroxyethyl]morpholin-4-yl]pyridazin-3-yl]-3,5-dimethyl-phenol

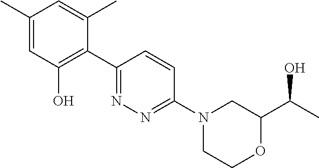 [SMILES: Cc1cc(C)c(-c2ccc(N3CCOC([C@H](C)O)C3)nn2)c(O)c1]

### Int 98.1 and Int 99.1: 2 diastereoisomer of (1S)-1-[4-(6-chloropyridazin-3-yl)morpholin-2-yl]ethanol

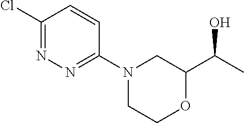 [SMILES: C[C@H](O)C1CN(c2ccc(Cl)nn2)CCO1]

In a microwave vial, to a solution of 3,6-dichloropyridazine (0.6 mmol, 90 mg) in 3 ml of NMP were added (1S)-1-morpholin-2-ylethanol hydrochloride (0.6 mmol, 100 mg) and DIPEA (0.8 mmol, 0.14 ml). The vial was sealed, and the mixture was heated at 150° C. under microwave irradiation for 1 h. After cooling down, the solution was concentrated under reduced pressure. The residue was purified by flash chromatography on silica gel using Heptane/EOAct (from 100/0 to 70/30) to give the title compound (64 mg, 44%) as a mixture of 2 diastereoisomers:

LCMS (Method S): Rt=0.81 min; MS m/z [M+H]⁺ 244.2

### 2 diastereoisomers of 2-[6-[2-[(1S)-1-hydroxyethyl]morpholin-4-yl]pyridazin-3-yl]-3,5-dimethyl-phenol (Examples 98 and 99)

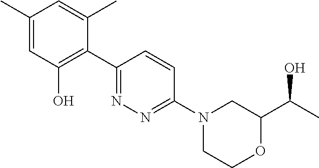 [SMILES: Cc1cc(C)c(-c2ccc(N3CCOC([C@H](C)O)C3)nn2)c(O)c1]

In a microwave vial under Ar, to a mixture of (1S)-1-[4-(6-chloropyridazin-3-yl)morpholin-2-yl]ethanol (0.21 mmol, 51 mg) and 3,5-dimethyl-2-(4,4,5,5-tetramethyl-1,3,2-dioxaborolan-2-yl)phenol (0.21 mmol, 52 mg) in 0.8 ml of 1,4-dioxane and 0.2 ml of water was added K₃PO₄ (0.63 mol, 135 mg). The solution was purged with Ar for 10 min, then Pd-118 (also named Pd-118 or PdCl₂(dtbpf)) (0.02 mmol, 15 mg) was added. The mixture was heated at 120° C. for 18 h. After cooling down, the mixture was filtered through a Celite® pad. Water and EtOAc were added to the filtrate. The aqueous layer was extracted with EtOAc. The combined organic layers were dried over Na₂SO₄, filtered and concentrated under reduced pressure. The residue was purified by reverse phase preparative LC (Column YMC—Actus Triart Prep C18-S 150*30 mm 5 μm Flow rate 50 ml/min-Isocratic mode MeCN/aq·NH₄HCO₃ 0.2% pH=7.9 28/72) to give separately 2 diastereoisomers, first eluting example 99 (Diastereoisomer 1, 12 mg, 17%) and second eluting example 98 (Diastereoisomer 2, 10 mg, 15%).

### Examples 108 and 180: 2 diastereoisomers of 2-[6-[2-[(1R)-1-hydroxyethyl]morpholin-4-yl]pyridazin-3-yl]-3,5-dimethyl-phenol

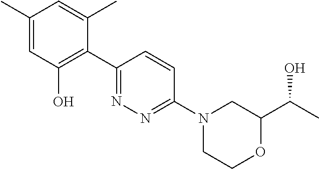 [SMILES: Cc1cc(C)c(-c2ccc(N3CCOC([C@@H](C)O)C3)nn2)c(O)c1]

### Int 108.1: 2 diastereoisomers of (1R)-1-[4-(6-chloropyridazin-3-yl)morpholin-2-yl]ethanol

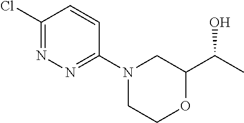 [SMILES: C[C@@H](O)C1CN(c2ccc(Cl)nn2)CCO1]

In a microwave vial, to a solution of 3,6-dichloropyridazine (0.6 mmol, 90 mg) in 3 ml of NMP were added (1R)-1-morpholin-2-ylethanol hydrochloride (0.6 mmol, 100 mg) and DIPEA (0.8 mmol, 0.14 ml). The vial was sealed, and the mixture was heated at 150° C. under microwave irradiation for 1 h. After cooling down, the solution was concentrated under reduced pressure. The residue was purified by flash chromatography on silica gel using Heptane/EtOAc (from 100/0 to 70/30) to give 2 diastereoisomers:

Diastereoisomer 1: (16 mg, 11% yield).

1H NMR (500 MHz, CDCl₃) δ (ppm): 7.23 (d, J=9.5 Hz, 1H), 6.92 (d, J=9.5 Hz, 1H), 4.20-4.13 (m, 1H), 4.12-4.09 (m, 1H), 4.08-4.04 (m, 1H), 3.93 (dt, J=11.2, 5.6 Hz, 1H), 3.73 (td, J=11.6, 2.8 Hz, 1H), 3.46 (ddd, J=10.5, 4.8, 2.8 Hz, 1H), 3.17-3.07 (m, 1H), 3.01 (dd, J=12.9, 10.5 Hz, 1H), 2.18-2.06 (m, 1H), 1.27 (d, J=6.5 Hz, 3H).

Diastereoisomer 2: (24 mg, 17% yield).

1H NMR (500 MHz, CDCl₃) δ (ppm): 7.24 (d, J=9.5 Hz, 1H), 6.90 (d, J=9.5 Hz, 1H), 4.18 (dt, J=13.0, 2.0 Hz, 1H), 4.16-4.08 (m, 1H), 4.03-3.96 (m, 1H), 3.82-3.75 (m, 1H), 3.75-3.69 (m, 1H), 3.38 (ddd, J=10.7, 6.3, 2.8 Hz, 1H), 3.10 (td, J=12.3, 3.6 Hz, 1H), 2.87 (dd, J=12.8, 10.8 Hz, 1H), 2.47 (d, J=3.8 Hz, 1H), 1.26 (d, J=6.5 Hz, 3H).

### 2 diastereoisomers of 2-[6-[2-[(1R)-1-hydroxyethyl]morpholin-4-yl]pyridazin-3-yl]-3,5-dimethyl-phenol (Examples 108 and 180)

### Diastereoisomer 1 (Example 108)

In a microwave vial under Ar, to a solution of (1R)-1-[4-(6-chloropyridazin-3-yl)morpholin-2-yl]ethanol (Diastereoisomer 2 previous Example 108.1, 0.1 mmol, 24 mg) and 3,5-dimethyl-2-(4,4,5,5-tetramethyl-1,3,2-dioxaborolan-2-yl)phenol (0.11 mmol, 27 mg,) in 0.8 ml of 1,4-dioxane and 0.2 ml of water was added K₃PO₄ (0.3 mol, 63 mg). The solution was purged with Ar for 10 min, then Pd-118 (0.01 mmol, 7 mg) was added. The mixture was heated at 120° C. for 18 h. After cooling down, the mixture was filtered through a Celite® pad. Water and EtOAc were added to the filtrate. The organic layer was separated, dried over MgSO₄, filtered and concentrated under reduced pressure. The residue was purified by reverse phase preparative LC (Column YMC-Actus Triart Prep C18-S 150*30 mm 5 μm Flow rate 50 ml/min-Focused gradient MeCN/aq·NH₄HCO₃ 0.2% pH=7.9 Focused gradient from 20/80 to 55/45 15CV), to give after lyophilization the title compound as a white solid (6 mg, 18% yield).

### Diastereoisomer 2 (Example 180)

In a microwave vial under Ar, to a solution of (1R)-1-[4-(6-chloropyridazin-3-yl)morpholin-2-yl]ethanol (Diastereoisomer 1 previous Example 108.1, 0.115 mmol, 24 mg) and 2-hydroxy-4,6-dimethyl-phenyl)boronic acid (0.172 mmol, 29 mg,) in 1 ml of 1,4-dioxane and 0.25 ml of water was added K₂CO₃ (0.23 mol, 46 mg). The solution was purged with Ar for 10 min, then XPhos (0.00575 mmol, 2.74 mg) and Xphos Pd G2 (0.00575 mmol, 4.52 mg) are added. The mixture was purged again with N₂ and heated at 120° C. for 18 h. After cooling down, the mixture was filtered through a Celite® pad and concentrated under reduced pressure.

The residue was purified by by flash chromatography on silica gel using DCM/MeOH (from 100/0 to 97/3) to give, after lyophilization, the title compound (7.3 mg, 19%).

### Example 82: 2-[6-[(3S)-3-(methoxymethyl)pyrrolidin-1-yl]pyridazin-3-yl]-3,5-dimethyl-phenol

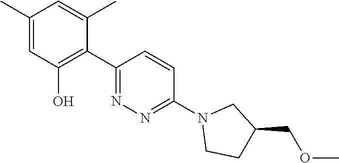

### Int 82.1: 3-chloro-6-[(3S)-3-(methoxymethyl)pyrrolidin-1-yl]pyridazine

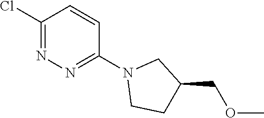

In a microwave vial, to a solution of 3,6-dichloropyridazine (2.68 mmol, 400 mg) in 10 ml of NMP are added DIPEA (2.68 mmol, 0.47 ml) and (3S)-3-(methoxymethyl)pyrrolidine (2.60 mol, 300 mg). The vial was sealed, and the mixture was heated at 150° C. under microwave irradiation for 1 h30. After cooling down, the solution was concentrated under reduced pressure.

The residue was purified by flash chromatography on silica gel using Heptane/EtOAc (from 100/0 to 30/70) to give the title compound as a white solid (450 mg, 75% yield).

1H NMR (400 MHz, DMSO-d₆) δ (ppm): 7.46 (d, J=9.5 Hz, 1H), 6.98 (d, J=9.5 Hz, 1H), 3.59-3.49 (m, 2H), 3.46-3.35 (m, 2H), 3.27 (s, 3H), 3.22-3.13 (m, 1H), 2.59-2.54 (m, 1H), 2.12-2.00 (m, 1H), 1.95-1.85 (m, 1H), 1.81-1.67 (m, 1H).

### 2-[6-[(3S)-3-(methoxymethyl)pyrrolidin-1-yl]pyridazin-3-yl]-3,5-dimethyl-phenol (Example 82)

In a microwave vial, under Ar, to a solution of 3-chloro-6-[(3S)-3-(methoxymethyl)pyrrolidin-1-yl]pyridazine (0.44 mmol, 100 mg,) in 1 ml of 1,4-dioxane and 0.25 ml of water were added. K₃PO₄ (1.32 mmol, 280 mg,) and 3,5-dimethyl-2-(4,4,5,5-tetramethyl-1,3,2-dioxaborolan-2-yl)phenol (0.44 mol, 110 mg,). The solution was purged with Ar for 10 min, then Pd-118 (0,048 mmol, 31 mg) was added. The mixture was heated at 120° C. for 18 h. After cooling down, the mixture was filtered through a Celite® pad. Water and EtOAc were added to the filtrate. The organic layer was separated, dried over MgSO₄, filtered and concentrated under reduced pressure.

The residue was purified by flash chromatography on silica gel using Heptane/EtOAc (from 100/0 to 70/30). The fractions containing the compound are combined, evaporated in vacuo and taken-up with MeCN (1 ml), extended with water (3 ml), then freeze-dried over 18 h to give the title compound as a white powder (20 mg, 15%).

Compound Example 111 was synthesized analogously to Example 82.

Compound Example 88 was synthesized analogously to Example 82 using BuOH instead of NMP at 120° C. for n-arylation.

### Example 31: 2-[6-[(3R)-3-amino-1-piperidyl]pyridazin-3-yl]-3,5-dimethyl-phenol; formic acid

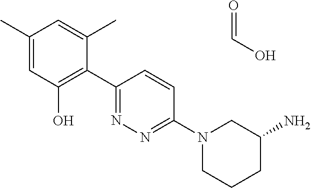 [SMILES: Cc1cc(C)c(-c2ccc(N3CCC[C@@H](N)C3)nn2)c(O)c1 ; O=CO]

### Int 31.1: tert-butyl N-[(3R)-1-[6-(2-hydroxy-4,6-dimethyl-phenyl)pyridazin-3-yl]-3-piperidyl]carbamate

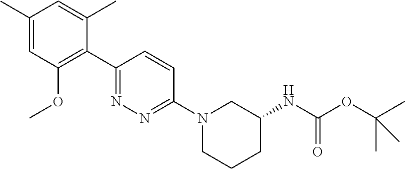 [SMILES: COc1cc(C)cc(C)c1-c1ccc(N2CCC[C@@H](NC(=O)OC(C)(C)C)C2)nn1]

To a solution of (R)-3-BOC-aminopiperidine (2.2 mmol, 218 mg) and Et₃N (5.03 mmol, 0.7 ml) in 10 ml of ethanol was added 3,6-dichloropyridazine (2.01 mmol, 300 mg) and the mixture was warmed to reflux for 16 h. After cooling to rt were added 10 ml of 1,4-dioxane, 2 ml of water, Cesium carbonate (3.0206 mmol, 985 mg) and (2-methoxy-4,6-dimethylphenyl)boronic acid (2.21 mmol, 400 mg). The solution was purged with Ar for 10 min, then was added [1,1′-bis(diphenylphosphino)ferrocene]dichloropalladium(II) (0.2 mmol, 150 mg) and the mixture was warmed to 120° C. for 4 h. After cooling to rt, the mixture was concentrated under reduced pressure. EtOAc was added and the resulting mixture was washed with water and brine. The organic layer was dried over Na₂SO₄, filtered and concentrated under reduced pressure.

The residue was purified by flash chromatography on silica gel using cyclohexane/EtOAc (from 100/0 to 0/100) to give the title compound as a white foam (218 mg, 26% yield). LCMS (Method I): Rt=1.54 min; MS m/z [M+H]⁺ 413

### 2-[6-[(3R)-3-amino-1-piperidyl]pyridazin-3-yl]-3,5-dimethyl-phenol; formic acid

### Example 31

At 0° C., to a solution of tert-butyl N-[(3R)-1-[6-(2-hydroxy-4,6-dimethyl-phenyl)pyridazin-3-yl]-3-piperidyl]carbamate (0.51 mmol, 210 mg) in 20 ml of DCM was slowly added a 1N solution of boron tribromide in DCM (1.1 mmol, 1.1 ml) and the mixture was stirred at rt for 3 h. Then MeOH was slowly added, and the solution was stirred 20 min. The resulting mixture was concentrated under reduced pressure and purified by prep-HPLC (column: Sunfire prep C18 OBD; 100*30 mm*10 μm; mobile phase: [water (TFA 10⁻² N)-ACN]; B %: 10%-80% in 18 min. at 50 ml/min) (twice purification) to give the title compound as a light yellow solid (12 mg, 7% yield).

Compound Example 7 was synthesized analogously to Example 31.

### Example 105: N-[[(2S)-4-[6-(2-hydroxy-4,6-dimethyl-phenyl)pyridazin-3-yl]morpholin-2-yl]methyl]acetamide

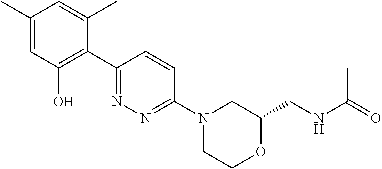 [SMILES: CC(=O)NC[C@H]1CN(c2ccc(-c3c(C)cc(C)cc3O)nn2)CCO1]

### Int 105.1: tert-butyl N-[[(2S)-4-(6-chloropyridazin-3-yl)morpholin-2-yl]methyl]carbamate

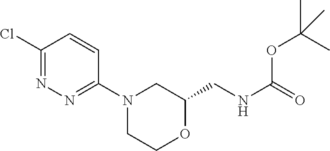 [SMILES: CC(C)(C)OC(=O)NC[C@H]1CN(c2ccc(Cl)nn2)CCO1]

In a microwave vial, to a solution of 3,6-dichloropyridazine (1.76 mmol, 276 mg) in 5 ml of 1-butanol were added (R)-tert-butyl (morpholin-2-ylmethyl) carbamate (1.76 mmol, 400 mg) and Et₃N (3.9 mmol, 0.55 ml). The vial was sealed, and the mixture was heated at 180° C. under microwave irradiation for 3 h. After cooling down the solution, the mixture was concentrated under reduced pressure, ethyl acetate was added, and the solution was washed with water (3 times) and brine. The organic layer was dried over MgSO₄, filtered and concentrated under reduced pressure. The residue was purified by flash chromatography on silica gel using DCM/MeOH/ACN (from 100/0/0 to 96/2/2) to give the title compound as a light yellow solid (268 mg, 46% yield).

LCMS (Method G): Rt=1.75 min; MS m/z [M+H]⁺ 329

### Int 105.2: [(2S)-4-(6-chloropyridazin-3-yl)morpholin-2-yl]methanamine; hydrochloride

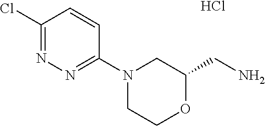 [SMILES: Cl ; NC[C@H]1CN(c2ccc(Cl)nn2)CCO1]

To a solution of tert-butyl N-[[(2S)-4-(6-chloropyridazin-3-yl)morpholin-2-yl]methyl]carbamate (0.39 mmol, 130 mg) in 3 ml of dichloromethane was added 1 ml of a 4M solution of hydrochloric acid in 1,4-dioxane. The solution was stirred for 3 h30 at room temperature. The mixture was concentrated under reduced pressure, then 1 ml of MeOH was added and the solution was concentrated under reduced pressure (3 times) to give the titled compound as an oil (145 mg), was used without further purification in the next step.

LCMS (Method G): Rt=0.21 min; MS m/z [M+H]⁺ 229

### Int 105.3: N-[[(2S)-4-(6-chloropyridazin-3-yl) morpholin-2-yl]methyl]acetamide

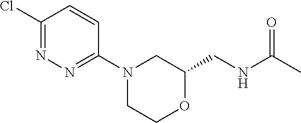 [SMILES: CC(=O)NC[C@H]1CN(c2ccc(Cl)nn2)CCO1]

To a solution of [(2S)-4-(6-chloropyridazin-3-yl)morpholin-2-yl]methanamine; hydrochloride (0.39 mmol, 104 mg) in 2 ml of DCM were added Et₃N (1.0 mmol, 0.14 ml) and acetic anhydride (0.60 mmol, 0.06 ml). The mixture was stirred for 2 h30 at room temperature. Then dichloromethane was added, and the mixture was washed with saturated aqueous NaHCO₃ solution (3 times). The organic layer was dried over Na₂SO₄, filtered and concentrated under reduced pressure to give the titled compound as a solid (89 mg, 83% yield).

LCMS (method L): Rt=1.36 min; MS m/z [M+H]⁺ 271

### N-[[(2S)-4-[6-(2-hydroxy-4,6-dimethyl-phenyl)pyridazin-3-yl]morpholin-2-yl]methyl]acetamide (Example 105)

In a microwave vial, under Ar, to a solution of (2-hydroxy-4,6-dimethyl-phenyl)boronic acid (0.43 mmol, 72 mg) and N-[[(2S)-4-(6-chloropyridazin-3-yl)morpholin-2-yl]methyl]acetamide (0.34 mmol, 90 mg) in 2 ml of 1,4-dioxane was added a solution of sodium carbonate (1 mmol, 85 mg) in 0.5 ml of water. The mixture was purged with Ar for 10 min, then Pd(PPh₃)₄ (0.017 mmol, 20 mg) was added. The vial was sealed, and the mixture was heated at 110° C. under microwave irradiation for 1 h. After cooling down the solution, the reaction mixture was filtered on a pad of Decalite® and rinsed with ethyl acetate. The filtrate was concentrated under reduced pressure, EtOAc was added, and the resulting mixture was washed with water (3 times) and brine. The organic layer was dried over Na₂SO₄, filtered and concentrated under reduced pressure. The residue was purified by flash chromatography on silica gel using DCM/MeOH/ACN (from 100/0/0 to 96/2/2) to give the title compound as a white solid (47 mg, 39% yield).

Compounds Examples 106, 211, 212, 216, 267, 275, 276, 279, 280 and 285 were synthesized analogously to Example 105.

### Example 67: (3S)-1-[6-(2-hydroxy-4,6-dimethyl-phenyl)pyridazin-3-yl]piperidin-3-ol

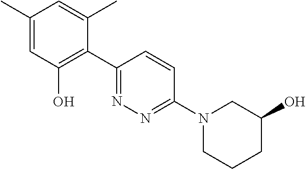 [SMILES: Cc1cc(C)c(-c2ccc(N3CCC[C@H](O)C3)nn2)c(O)c1]

### Int 67.1: (3S)-1-(6-chloropyridazin-3-yl)piperidin-3-ol

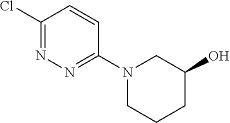 [SMILES: O[C@H]1CCCN(c2ccc(Cl)nn2)C1]

A solution of 3,6-dichloropyridazine (4.7 mmol, 700 mg), (3S)-piperidin-3-ol; hydrochloride (5.17 mmol, 711 mg) and Et₃N (9.4 mmol, 1.31 ml) in 11 ml of NMP was stirred at 130° C. for 5 h. The mixture was cooled down, diluted with EtOAc and washed with water. The aqueous layer was extracted with EtOAc (4 times), and the combined organic layers were dried over Na₂SO₄, filtered and concentrated under reduced pressure. The residue was purified by flash chromatography on silica gel using cyclohexane/EtOAc (10/0 to 0/10) to give the title compound as a yellow oil (500 mg, 50% yield).

LCMS (method I): Rt=0.96 min; MS m/z [M+H]⁺ 214

### Int 67.2: (3S)-1-[6-(2-methoxy-4,6-dimethyl-phenyl)pyridazin-3-yl]piperidin-3-ol

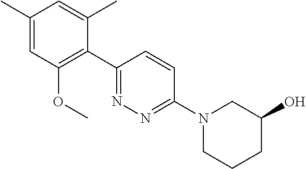 [SMILES: COc1cc(C)cc(C)c1-c1ccc(N2CCC[C@H](O)C2)nn1]

Under Ar, to a solution of (2-methoxy-4,6-dimethyl-phenyl)boronic acid (2.47 mmol, 445 mg) and (3S)-1-(6-chloropyridazin-3-yl)piperidin-3-ol (2.25 mmol, 480 mg) in 8 ml of 1,4-dioxane and 2 ml of water were added cesium carbonate (4.5 mmol, 1.46 g) and then [1,1′-bis(diphenylphosphino)ferrocene]dichloropalladium(II) (0.22 mmol, 164 mg). The mixture was stirred at 100° C. for 2.5 h, then another 85 mg (0.47 mmol) of (2-methoxy-4,6-dimethyl-phenyl)boronic acid and 150 mg (0.46 mmol) of cesium carbonate were added and the mixture was stirred at 100° C. for another 3 h. The mixture was cooled down, diluted with EtOAc and washed with 5% citric acid aqueous solution. The organic layer was dried over Na₂SO₄, filtered and concentrated under reduced pressure. The residue was triturated in diethylether and filtered off to give the title compound as a yellow solid (335 mg, 48% yield). LCMS (Method I): Rt=1.13 min; MS m/z [M+H]⁺ 314;

### (3S)-1-[6-(2-hydroxy-4,6-dimethyl-phenyl)pyridazin-3-yl]piperidin-3-ol (Example 67)

Under Ar, a solution of (3S)-1-[6-(2-methoxy-4,6-dimethyl-phenyl)pyridazin-3-yl]piperidin-3-ol (1.07 mmol, 335 mg) in 15 ml of DCM was cooled down to 0° C. A solution of TN boron tribromide in DCM (2.14 mmol, 2.14 ml) was slowly added. The mixture was stirred at rt for 1 h, then quenched by slow addition of 3 ml of MeOH. The resulting solution was stirred for 20 min then concentrated under reduced pressure. The residue was purified by flash chromatography on silica gel using DCM/MeOH (100/0 to 95/5) purified by silica gel chromatography eluting with a gradient of 0 to 5% of MeOH in DCM to give the title compound as a white solid (192 mg, 60% yield).

### Example 121: 2-[6-[(2S)-2-(hydroxymethyl)morpholin-4-yl]pyridazin-3-yl]-3-methyl-5-(trifluoromethoxy)phenol

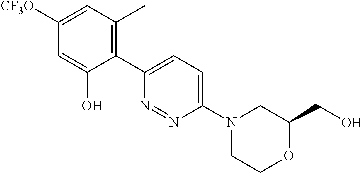

### 2-[6-[(2S)-2-(hydroxymethyl)morpholin-4-yl]pyridazin-3-yl]-3-methyl-5-(trifluoromethoxy)phenol (Example 121)

In a microwave vial under Ar, to a mixture of [(2S)-4-(6-chloropyridazin-3-yl)morpholin-2-yl]methanol (Int 8.1) (0.287 mmol, 91.4 mg) in 1 ml of 1,4-dioxane was added 3-methyl-2-(4,4,5,5-tetramethyl-1,3,2-dioxaborolan-2-yl)-5-(trifluoromethoxy)phenol (0.287 mmol, 66.0 mg) and K₃PO₄ (0.862 mmol, 183 mg,) in 0.25 ml of water. The mixture was purged with N₂. Then Pd¹¹⁸ (0.0316 mmol, 20.6 mg,) was added and purged again with N₂. The mixture was heated at 120° C. for 24 hours. The reaction mixture was quenched with water and extracted with DCM. The organic layer was dried over MgSO₄, filtered, and evaporated to afford a residue which was purified by flash chromatography on silica gel using DCM/MeOH (from 100/0 to 90/10). The fractions containing the compound were combined and evaporated under reduced pressure to give a colorless oil (25 mg). This oil was purified by preparative HPLC (YMC—Actus Triart Prep C18-S 150*30 mm 5 μm, mobile phase: MeCN/aq·NH₄HCO₃ 0.2% pH=7.9; gradient from 25/75 to 65/35) to afford a white solid (11 mg), which was taken-up with MeCN (1 mL), extended with water (3 mL), then lyophilized to give the title compound as a white solid (10 mg, 9% yield).

### Example 125: 2-[6-[(4aR,7aR)-3,4,4a,5,7,7a-hexahydro-2H-pyrrolo[3,4-b][1,4]oxazin-6-yl]pyridazin-3-yl]-3,5-dimethylphenol

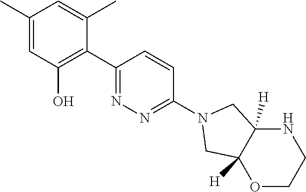 [SMILES: Cc1cc(C)c(-c2ccc(N3C[C@H]4NCCO[C@@H]4C3)nn2)c(O)c1]

### Int 125.1: tert-butyl (4aR,7aR)-6-(6-chloropyridazin-3-yl)-2,3,4a,5,7,7a-hexahydropyrrolo[3,4-b][1,4]oxazine-4-carboxylate

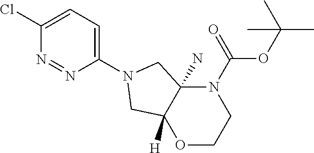 [SMILES: CC(C)(C)OC(=O)N1CCO[C@@H]2CN(c3ccc(Cl)nn3)C[C@]21N]

In a microwave vial, tert-butyl (4aR,7aR)-3,4a,5,6,7,7a-hexahydro-2H-pyrrolo[3,4-b][1,4]oxazine-4-carboxylate (1.01 mmol, 0.230 g), 3,6-dichloropyridazine (1.01 mmol, 0.150 g) and DIPEA (1.01 mmol, 0.175 ml) in 5 ml of butan-1-ol was heated at 120° C. using one single mode microwave (Biotage Initiator EXP 60) with a power output ranging from 0 to 400 W for 2 h. After cooling, the reaction mixture was concentrated under reduced pressure and the residue was purified by flash chromatography on silica gel using Hept/AcOEt 100/0 to 30/70 to give the title compound as a white solid (224 mg, 65% yield).

LCMS (method R): Rt=1.29 min; MS m/z [M+H]⁺=341.4

### Int 125.2: tert-butyl (4aR,7aR)-6-[6-(2-hydroxy-4,6-dimethyl-phenyl)pyridazin-3-yl]-2,3,4a,5,7,7a-hexahydropyrrolo[3,4-b][1,4]oxazine-4-carboxylate

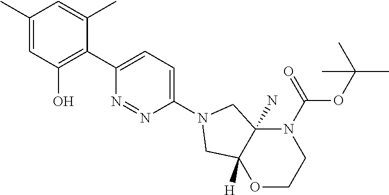 [SMILES: Cc1cc(C)c(-c2ccc(N3C[C@H]4OCCN(C(=O)OC(C)(C)C)[C@]4(N)C3)nn2)c(O)c1]

In a microwave vial under Ar, a mixture of tert-butyl (4aR,7aR)-6-(6-chloropyridazin-3-yl)-2,3,4a,5,7,7a-hexahydropyrrolo[3,4-b][1,4]oxazine-4-carboxylate (0.000205 mol, 0.0700 g), K₂CO₃ (0.000411 mol, 0.0568 g), (2-hydroxy-4,6-dimethyl-phenyl)boronic acid (0.000308 mol, 0.0511 g) in 1 ml of water and 4 ml of 1,4-dioxane was degassed under N₂ flow. XPhos Pd G2 (1.03e-5 mol, 0.00808 g) and XPhos (1.03e-5 mol, 0.00490 g) were added and the mixture was heated at 100° C. for 18 h. After cooling at rt, the reaction mixture was filtered through a Celite® pad, water was added. The organic layer was extracted with AcOEt, separated, dried over MgSO₄, filtered, and concentrated under reduced pressure. The residue was purified by flash chromatography on silica gel using DCM/MeOH 100/0 to 95/5 to give the title compound as a yellow oil (65 mg, 72% yield).

LCMS (Method S): Rt=1.34 min; MS m/z [M+H]⁺=427.4

### 2-[6-[(4aR,7aR)-3,4,4a,5,7,7a-hexahydro-2H-pyrrolo[3,4-b][1,4]oxazin-6-yl]pyridazin-3-yl]-3,5-dimethylphenol (Example 125)

To a mixture of tert-butyl (4aR,7aR)-6-[6-(2-hydroxy-4,6-dimethyl-phenyl)pyridazin-3-yl]-2,3,4a,5,7,7a-hexahydropyrrolo[3,4-b][1,4]oxazine-4-carboxylate (0.000152 mol, 0.0650 g) in 3 ml of dichloromethane was added TFA (0.00152 mol, 0.174 g). The mixture was then stirred 18 h at rt. The mixture was concentrated under reduced pressure. The residue was taken up in water and basified with a 10% aqueous solution of K₂CO₃, then extracted with DCM, dried over MgSO₄, filtered, and concentrated under reduced pressure. The residue was purified by flash chromatography on silica gel using DCM/MeOH 100/0 to 95/5 to give the title compound as a white solid (20 mg, 40% yield).

### Example 126: 1-[4-[6-(2-hydroxy-4,6-dimethylphenyl)pyridazin-3-yl]morpholin-2-yl]propan-1-one

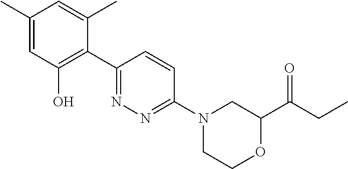 [SMILES: CCC(=O)C1CN(c2ccc(-c3c(C)cc(C)cc3O)nn2)CCO1]

### Int 126.1: 1-[4-(6-chloropyridazin-3-yl)morpholin-2-yl]cyclopropanol

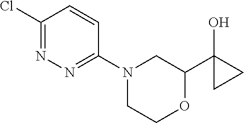 [SMILES: OC1(C2CN(c3ccc(Cl)nn3)CCO2)CC1]

In a microwave vial, 1-morpholin-2-ylcyclopropanol hydrochloride (1.39 mmol, 250 mg), DIPEA (3.15 mmol, 0.539 ml) and 3,6-dichloropyridazine (1.57 mmol, 234 mg) were dissolved in 3.5 ml of NMP. The mixture was then heated at 150° C. using one single mode microwave (Biotage Initiator EXP 60) with a power output ranging from 0 to 400 W for 2 hours. After cooling to rt, the reaction mixture was concentrated under reduced pressure and the residue was purified by flash chromatography on silica gel using Hept/AcOEt 100/0 to 50/50, then 30/70 to give the title compound as a pale yellow solid (231 mg, 65% yield).

LCMS (Method R): Rt=0.97; MS m/z [M+H]⁺=256.2;

### 1-[4-[6-(2-hydroxy-4,6-dimethylphenyl)pyridazin-3-yl]morpholin-2-yl]propan-1-one (Example 126)

In a microwave vial under Ar, to a mixture of 1-[4-(6-chloropyridazin-3-yl)morpholin-2-yl]cyclopropanol (0.903 mmol, 231 mg) in 8 ml of 1,4-dioxane was added 3,5-dimethyl-2-(4,4,5,5-tetramethyl-1,3,2-dioxaborolan-2-yl)phenol (0.903 mmol, 224 mg) and K₃PO₄ (2.71 mmol, 575 mg) in 2 ml of water. The mixture was purged with N₂. Then Pd¹¹⁸ (0.0994 mmol, 64.8 mg) was added and purged again with N₂. The mixture was then heated at 120° C. for 24 hours. The reaction mixture was quenched with water and extracted with DCM. The combined organic layers were dried over MgSO₄, filtered, and concentrated under reduced pressure. The residue was purified by flash chromatography on silica gel using DCM/MeOH 100/0 to 90/10 to afford a colorless oil (57.6 mg). This oil was purified by preparative HPLC (YMC—Actus Triart Prep C18-S 150*30 mm 5 μm, mobile phase: MeCN/aq·NH₄HCO₃ 0.2% pH=7.9; gradient from 25/75 to 65/35) to afford a white solid (47 mg), which was triturated in DIPE. The precipitate was filtered and dried under reduced pressure to give the title compound as a white solid (20 mg).

### Example 156 and 157: 1-[(2R)-4-[6-(2-hydroxy-4,6-dimethylphenyl)pyridazin-3-yl]morpholin-2-yl]propan-1-one and 1-[(2S)-4-[6-(2-hydroxy-4,6-dimethylphenyl) pyridazin-3-yl]morpholin-2-yl]propan-1-one

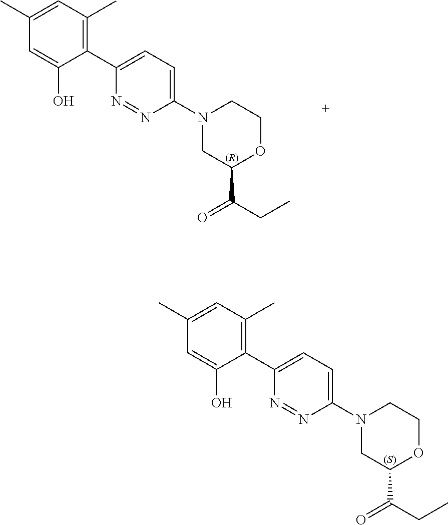

The filtrate and excess product of 1-[4-[6-(2-hydroxy-4,6-dimethylphenyl)pyridazin-3-yl]morpholin-2-yl]propan-1-one (Example 126) was purified by preparative chiral SFC (column: LuxCell-4 20×250 mm, Mobile phase: CO₂/(MeOH+0.3% iPrNH₂) 65/35). The fractions containing compound were combined and evaporated in vacuo to give 20 mg of a first fraction and 19 mg of a second fraction. Both fractions were lyophilized to give respectively 17 mg of first enantiomer Example 156 and 16 mg of second enantiomer Example 157 (The absolute configurations cannot be assigned).

### Example 141: 2-[6-[(2S)-2-(hydroxymethyl)morpholin-4-yl]pyridazin-3-yl]-3,6-dimethylphenol

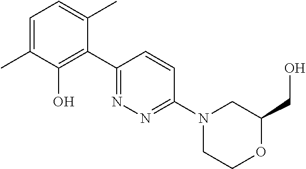 [SMILES: Cc1ccc(C)c(-c2ccc(N3CCO[C@H](CO)C3)nn2)c1O]

A vial was loaded with [(2S)-4-(6-chloropyridazin-3-yl)morpholin-2-yl]methanol (0.327 mmol, 75.0 mg), potassium carbonate (0.653 mmol, 90.3 mg,), XPhos Pd G2 (0.0163 mmol, 12.8 mg) and XPhos (0.0163 mmol, 7.78 mg), then purged with nitrogen. A solution of compound 3,6-dimethyl-2-(4,4,5,5-tetramethyl-1,3,2-dioxaborolan-2-yl)phenol (0.490 mmol, 122 mg) in 2 ml of dioxane and 0.4 ml of water wee added. The resulting mixture was then stirred at 100° C. for 18 h. An aqueous solution of HCl 0.05 M was added to the mixture and the product was extracted with DCM (5 times). The aqueous layer was then neutralized to pH=7 with a solution of 1% sodium bicarbonate and washed with DCM (5 times). The combined organic layers were dried over MgSO₄ and concentrated under reduced pressure. The residue was purified by flash chromatography on silica gel using Hept/AcOEt 100/0 to 0/100, then 30/70 to give the title compound as a white solid (51 mg, 50% yield).

Compounds Examples 152, 153 and 191 were synthesized analogously to Example 141.

### Example 142 and 143: 2- [(2R)-4-[6-(2-hydroxy-4,6-dimethylphenyl)pyridazin-3-yl]morpholin-2-yl]-N-methylacetamide and 2-[(2S)-4-[6-(2-hydroxy-4,6-dimethylphenyl)pyridazin-3-yl]morpholin-2-yl]-N-methylacetamide

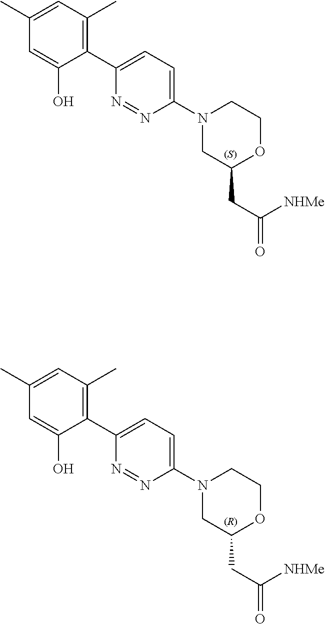

### Int 142.1: methyl 2-[4-(6-chloropyridazin-3-yl)morpholin-2-yl]acetate

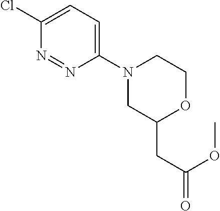 [SMILES: COC(=O)CC1CN(c2ccc(Cl)nn2)CCO1]

In a microwave vial, a mixture of 3,6-dichloropyridazine (0.00767 mol, 1.14 g), methyl 2-morpholin-2-ylacetate hydrochloride (0.00767 mol, 1.50 g) and DIPEA (0.0115 mol, 1.49 g) in 10 ml of butan-1-ol was heated at 120° C. using one single mode microwave (Biotage Initiator EXP 60) with a power output ranging from 0 to 400 W for 2 hours. After cooling to rt, the mixture was concentrated under reduced pressure and was purified by flash chromatography on silica gel using Hept/AcOEt 100/0 to 30/70 to give the title compound as a yellow oil (1.25 g, 60% yield).

LCMS (Method R): Rt=1.08; MS m/z [M+H]+=272.2

### Int 142.2: methyl 2-[4-[6-(2-hydroxy-4,6-dimethyl-phenyl)pyridazin-3-yl]morpholin-2-yl]acetate

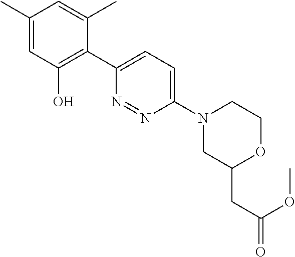

In a microwave vial, under Ar, was added methyl 2-[4-(6-chloropyridazin-3-yl)morpholin-2-yl]acetate (2.18 mmol, 592 mg), (2-hydroxy-4,6-dimethyl-phenyl)boronic acid (3.27 mmol, 542 mg), XPhos (0.109 mmol, 52.0 mg) and K₂CO₃ (4.36 mmol, 604 mg) in 8 ml of 1,4-dioxane and 2 ml of water. The mixture was purged with N₂, then XPhos Pd G₂ (0.109 mmol, 85.9 mg) was added and the mixture was heated at 90° C. for 18 h. After cooling to rt, the reaction mixture was quenched with water and extracted with EtOAc. The organic layer was dried over MgSO₄, filtered and concentrated under reduced pressure. The residue was purified by flash chromatography on silica gel using DCM/MeOH 100/0 to 95/05 to give the title compound as a yellow oil (697 mg, 90% yield).

LCMS (Method R): Rt=1.24 min; MS m/z [M+H]+=358.3

### Int 142.3: 2-[4-[6-(2-hydroxy-4,6-dimethyl-phenyl)pyridazin-3-yl]morpholin-2-yl]acetic acid

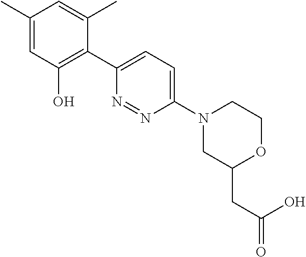 [SMILES: Cc1cc(C)c(-c2ccc(N3CCOC(CC(=O)O)C3)nn2)c(O)c1]

To a solution of methyl 2-[4-[6-(2-hydroxy-4,6-dimethyl-phenyl)pyridazin-3-yl]morpholin-2-yl]acetate (1.95 mmol, 696 mg) in 7 ml of tetrahydrofuran and 2.3 ml of water was added LiOH monohydrate (5.84 mmol, 245 mg). The reaction mixture was stirred at rt for 18 hours. The solvent was removed under reduced pressure. The residue was taken up in water, then HCl (3M) was added. After filtration, the filtrate was concentrated under reduced pressure to give the title compound as an orange solid (751 mg).

Product was used crude for next step.

¹H NMR (400 MHz, DMSO-d₆) δ (ppm): 12.39 (s, 1H), 10.10 (s, 1H), 7.94 (s, 2H), 6.72 (s, 1H), 6.66 (s, 1H), 4.35 (d, J=13.0 Hz, 1H), 4.17 (d, J=13.1 Hz, 1H), 4.08-3.95 (m, 1H), 3.91 (dt, J=7.1, 3.5 Hz, 1H), 3.70-3.59 (m, 1H), 3.17 (t, J=12.3 Hz, 1H), 3.00-2.89 (m, 1H), 2.58 (dd, J=15.8, 4.8 Hz, 1H), 2.45 (dd, J=15.8, 7.9 Hz, 1H), 2.26 (s, 3H), 2.10 (s, 3H).

LCMS (Method R): Rt=0.92 min; purity 94%, MS m/z (M+H)⁺=344.3

### 2-[(2R)-4-[6-(2-hydroxy-4,6-dimethylphenyl)pyridazin-3-yl]morpholin-2-yl]-N-methylacetamide and 2-[(2S)-4-[6-(2-hydroxy-4,6-dimethylphenyl)pyridazin-3-yl]morpholin-2-yl]-N-methylacetamide (Example 142 and Example 143)

To a solution of 2-[4-[6-(2-hydroxy-4,6-dimethyl-phenyl)pyridazin-3-yl]morpholin-2-yl]acetic acid (0.582 mmol, 200 mg) in 2.5 ml of N,N-dimethylformamide was added methylamine in THF (2 M) (0.641 mmol, 0.320 mL), HATU (0.641 mmol, 244 mg) and DIPEA (0.874 mmol, 0.150 ml). The reaction mixture was stirred at room temperature for 18 hours. Methylamine in THF (2 M) (0.641 mmol, 0.320 ml) was added to the mixture and stirred for another 5 hours. Methylamine in THF (2 M) (0.641 mmol, 0.320 ml) was added to the mixture and stirred for another 3 h. The reaction mixture was quenched with water and extracted with EtOAc. The organic layer was dried over MgSO₄, filtered and concentrated under reduced pressure. The residue was purified by flash chromatography on silica gel using DCM/MeOH 100/0 to 95/05 to give 32 mg of a colorless oil. This oil purified by preparative chiral SFC (Stationary phase: Chiralpak® AD-3 4.6×100 mm, Mobile phase: CO₂/(iPrOH+0.3% iPrNH₂) 55/45). The fractions containing compound were combined and evaporated in vacuo to give 10 mg of a first fraction and 8 mg of a second fraction. Both fractions were lyophilized to give respectively 8.5 mg of first enantiomer Example 142 and 7.5 mg of second enantiomer Example 143 (The absolute configurations cannot be assigned).

### Example 142 (Enantiomer 1)

LCMS (Method E): Rt=2.25 min; MS m/z [M+H]+=357.4; e.e. (by analytical SFC)=1000%

### Example 143 (Enantiomer 2)

LCMS (Method E): Rt=2.25 min; MS m/z [M+H]+=357.4; e.e. (by analytical SFC)=100%

### Example 146 and 147: 2- [(2R)-4-[6-(2-hydroxy-4,6-dimethylphenyl)pyridazin-3-yl]morpholin-2-yl]acetamide and 2-[(2S)-4-[6-(2-hydroxy-4,6-dimethylphenyl)pyridazin-3-yl]morpholin-2-yl]acetamide

To a solution of 2-[4-[6-(2-hydroxy-4,6-dimethyl-phenyl)pyridazin-3-yl]morpholin-2-yl]acetic acid (Int 142.3) (0.582 mmol, 200 mg) in 2 ml of N,N-dimethylformamide was added HATU (1.16 mmol, 443 mg), TEA (2.33 mmol, 0.325 ml) and HMDS (1.46 mmol, 0.304 ml). The reaction mixture was stirred at room temperature for 18 hours, then quenched with NH₄Cl 10% and stirred at room temperature for 15 min. The organic layer was extracted with DCM. The organic layer was dried over MgSO₄, filtered and concentrated under reduced pressure. The residue was purified by flash chromatography on silica gel using DCM/MeOH 100/0 to 95/05 to give 136 mg of a yellowish solid.

This solid was purified by preparative chiral SFC (Stationary phase: Chiralpak® AS-3 4.6×100 mm, Mobile phase: CO₂/(MeOH+0.3% iPrNH₂) 75/25). The fractions containing compound were combined and evaporated in vacuo to give 48 mg of first enantiomer Example 146 and 45 mg of second enantiomer Example 147 (The absolute configurations cannot be assigned).

### Example 146 (Enantiomer 1)

LCMS (Method E): Rt=2.32 min; MS m/z [M+H]+=343.2; e.e. (by analytical SFC)=99.2%

### Example 147 (Enantiomer 2)

LCMS (Method E): Rt=2.32 min; MS m/z [M+H]+=343.2; e.e. (by analytical SFC)=98.8%

### Example 150 and 151: 2- [(2R)-4-[6-(2-hydroxy-4,6-dimethylphenyl)pyridazin-3-yl]morpholin-2-yl]-N,N-dimethylacetamide and 2-[(2S)-4-[6-(2-hydroxy-4,6-dimethylphenyl)pyridazin-3-yl]morpholin-2-yl]-N,N-dimethylacetamide

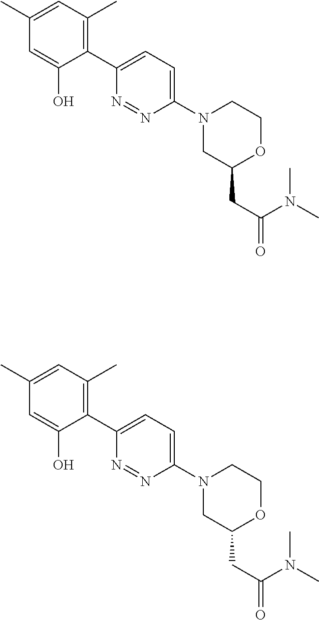

To a solution of 2-[4-[6-(2-hydroxy-4,6-dimethyl-phenyl)pyridazin-3-yl]morpholin-2-yl]acetic acid (Int 142.3) (0.495 mmol, 170 mg) in 2 ml of N,N-dimethylformamide were added Dimethylamine (2M) in THF (0.545 mmol, 0.272 ml), HATU (0.545 mmol, 207 mg) and DIPEA (0.743 mmol, 0.127 ml). The reaction mixture was stirred at room temperature for 18 hours, then quenched with water, and extracted with EtOAc. The organic layer was dried over MgSO₄, filtered, and concentrated under reduced pressure. The residue was purified by flash chromatography on silica gel using DCM/MeOH 100/0 to 95/05 to give 62 mg of a colorless oil.

This oil was purified by preparative chiral SFC (Stationary phase: Chiralpak® AD-3 4.6×100 mm, Mobile phase: CO₂/(iPrOH+0.3% iPrNH₂) 55/45). The fractions containing compound were combined and evaporated in vacuo to give, after lyophilization, 30 mg of first enantiomer Example 150 and 27 mg of second enantiomer Example 151 (The absolute configurations cannot be assigned).

### Example 150 (Enantiomer 1)

LCMS (Method E): Rt=2.59 min; MS m/z [M+H]+=371.3; e.e. (by analytical SFC)=100%

### Example 151 (Enantiomer 2)

LCMS (Method E): Rt=2.59 min; MS m/z [M+H]+=371.3; e.e. (by analytical SFC)=100%

### Example 165: 2-[6-[(2S)-2-[dideuterio(hydroxy)methyl]morpholin-4-yl]pyridazin-3-yl]-3,5-dimethylphenol

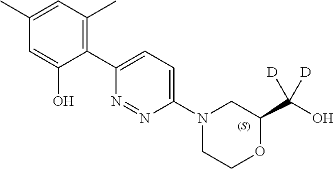

### Int 165.1: methyl (2S)-4-(6-chloropyridazin-3-yl)morpholine-2-carboxylate

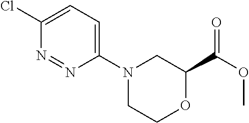 [SMILES: COC(=O)[C@@H]1CN(c2ccc(Cl)nn2)CCO1]

In a microwave vial, a mixture of methyl (2S)-morpholine-2-carboxylate hydrochloride (5.51 mmol, 1.00 g), DIPEA (8.26 mmol, 1.44 ml) and 3,6-dichloropyridazine (5.51 mmol, 0.820 g) in 12 ml of NMP was heated at 150° C. using one single mode microwave (Biotage Initiator EXP 60) with a power output ranging from 0 to 400 W for 1 h. After cooling to rt, the mixture was concentrated under reduced pressure and the residue was purified by reverse phase preparative LC (Cartridge YMC—DispoPack AT ODS_25 μm_300 g, Flow rate 65 ml/min, eluent MeCN/aq·NH₄HCO₃ 0.2% pH=7.9, gradient from 10/90 to 30/70). The fractions containing compound were concentrated under reduced pressure to give the title compound as a yellow oil (455 mg, 33% yield).

LCMS (Method T): Rt=4.16 min; MS m/z [M+H]+=257.8

### Int 165.2: [(2S)-4-(6-chloropyridazin-3-yl)morpholin-2-yl]-dideuterio-methanol

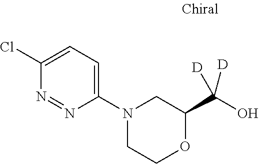 [SMILES: [2H]C([2H])(O)[C@@H]1CN(c2ccc(Cl)nn2)CCO1]

At 0° C. under Ar, to a stirred solution of methyl (2S)-4-(6-chloropyridazin-3-yl)morpholine-2-carboxylate (0.776 mmol, 200 mg) in 7 ml of MeOD was added NaBD₄ (1.55 mmol, 65.0 mg). The reaction mixture was stirred at rt for 8 h. After cooled down at 0° C., NaBD₄ (1.55 mmol, 65.0 mg) was added. Then, H₂O was slowly added, and the solution was extracted with DCM. The organic layer was washed with brine, dried over MgSO₄, then concentrated under reduced pressure. The residue was purified by flash chromatography on silica gel using DCM/MeOH 100/0 to 95/05 to give title compound as a colorless oil (110 mg, 62% yield).

LCMS (Method R): Rt=0.88 min; MS m/z [M+H]+=232.2;

### 2-[6-[(2S)-2-[dideuterio(hydroxy)methyl]morpholin-4-yl]pyridazin-3-yl]-3,5-dimethylphenol (Example 165)

In a microwave vial under Ar., To a solution of ([(2S)-4-(6-chloropyridazin-3-yl)morpholin-2-yl]-dideuterio-methanol (0.129 mmol, 30.0 mg) in 1.6 ml of 1,4-dioxane and 0.4 ml of water was added K₂CO₃ (0.259 mol, 35.8 mg) and (2-hydroxy-4,6-dimethyl-phenyl)boronic acid (1.94 mmol, 32.2 mg). The reaction mixture was degased with N₂ flow, then XPhos (0.00647 mmol, 3.09 mg) and XPhos Pd G2 (0.00647 mol, 5.09 mg) were added, and the mixture was heated at 90° C. for 18 h. After cooling to rt, the reaction mixture was concentrated under reduced pressure, then purified by flash chromatography on silica gel using DCM/MeOH 100/0 to 90/10. The residue obtained was lyophilized to give the title compound as a white solid (13.5 mg, 32% yield).

Compounds Examples 169 and 192 were synthesized analogously to Example 165.

### Example 168: 2-[6-[(4aR,7aR)-4-ethyl-2,3,4a,5,7,7a-hexahydropyrrolo[3,4-b][1,4]oxazin-6-yl]pyridazin-3-yl]-3,5-dimethylphenol

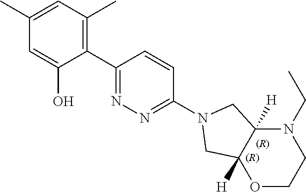

In sealed tube, to a stirred solution of 2-[6-[(4aR,7aR)-3,4,4a,5,7,7a-hexahydro-2H-pyrrolo[3,4-b][1,4]oxazin-6-yl]pyridazin-3-yl]-3,5-dimethylphenol (Example 125) (0.0551 mmol, 18.0 mg) in 2 ml of ethanol at rt were added acetaldehyde (0.08.27 mmol, 5.89 μl), acetic acid (0.0551 mmol, 4.03μ) and NaBH₃CN (0.110 mmol, 6.93 mg). The reaction mixture was stirred at 90° C. for 15 min. After cooling to 0° C., ice-cold water and saturated aqueous solution of NaHCO₃ were slowly added. The mixture was extracted with AcOEt. The organic layer was washed with water, then brine, dried over MgSO₄ and concentrated under reduced pressure. The residue was purified by flash chromatography on silica gel using DCM/MeOH 100/0 to 95/5 to give, after lyophilization, the title compound as a white solid (11.9 mg, 47% yield).

Compound Example 185 was synthesized analogously to Example 168.

### Example 181: (2S)-4-[6-(2-hydroxy-4,6-dimethylphenyl)pyridazin-3-yl]morpholine-2-carboxamide

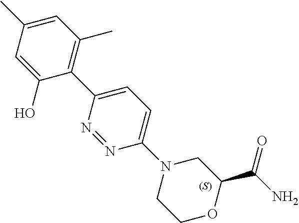

### Int 181.1: (2S)-4-(6-chloropyridazin-3-yl)morpholine-2-carboxylic acid

In a microwave vial, to a mixture of 3,6-dichloropyridazine (5.51 mmol, 820 mg) in 12 ml of NMP was added methyl (2S)-morpholine-2-carboxylate hydrochloride (5.51 mmol, 1.00 g) and DIPEA (1.44 ml, 8.26 mmol). The resulting mixture was heated at 150° C. for 1 h, using one single mode microwave (Biotage Initiator EXP 60) with a power output ranging from 0 to 400 W. After cooling to rt, the mixture was concentrated under reduced pressure. The residue was purified by reverse phase preparative LC (Cartridge YMC—DispoPack AT ODS_25 μm_300 g Flow rate 65 ml/min-Focused gradient MeCN/aq·NH₄HCO₃ 0.2% pH=7.9 Focused gradient from 10/90 to 30/70) to give the title compound (486 mg, 34%).

1H NMR (400 MHz, DMSO-d6) δ (ppm): 7.55-7.51 (m, 1H), 7.37 (d, J=9.7 Hz, 1H), 4.15-4.06 (m, 1H), 3.97 (dt, J=11.4, 3.6 Hz, 1H), 3.92-3.86 (m, 1H), 3.85-3.79 (m, 1H), 3.61-3.50 (m, 1H), 3.25-3.14 (m, 2H). (COOH not visible)

LCMS (Method T): Rt=1.71 min; MS m/z [M+H]+=244.1;

### Int 181.2: (2S)-4-(6-chloropyridazin-3-yl)morpholine-2-carboxamide Chiral

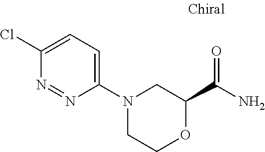 [SMILES: NC(=O)[C@@H]1CN(c2ccc(Cl)nn2)CCO1]

To a stirred solution of (2S)-4-(6-chloropyridazin-3-yl)morpholine-2-carboxylic acid (Int 181.1) (0.821 mmol, 200 mg) in 3 ml of DMF were added HATU (1.23 mmol, 468 mg), TEA (2.46 mmol, 0.343 ml) and HMDS (1.54 mmol, 0.321 ml). The reaction mixture was stirred at rt for 18 h. The mixture was quenched with water and extracted with DCM. The combined organic layers were washed with an aqueous solution of K₂CO₃ (10%) three times, then dried over MgSO₄, filtered, and concentrated under reduced pressure. The residue was purified by flash chromatography on silica gel using DCM/MeOH 100/0 to 95/5 to give, the title compound as a white solid (65 mg, 43% yield).

LCMS (Method T): Rt=2.73 min; MS m/z [M+H]+=242.8;

### (2S)-4-[6-(2-hydroxy-4,6-dimethylphenyl)pyridazin-3-yl]morpholine-2-carboxamide (Example 181)

In a microwave vial under Ar, a mixture of (2S)-4-(6-chloropyridazin-3-yl)morpholine-2-carboxamide (0.251 mmol, 61.0 mg), (2-hydroxy-4,6-dimethyl-phenyl)boronic acid (0.377 mol, 62.6 mg), K₂CO₃ (0.503 mmol, 69.5 mg) in 1.6 ml of 1,4-dioxane and 0.4 ml of water was degassed under N₂. XPhos (0.0126 mmol, 6.0 mg) and XPhos Pd G2 (0.0126 mmol, 9.9 mg) were added, and the mixture was heated at 90° C. for 3 h. The reaction mixture was then cooling down and concentrated under reduced pressure.

The residue was purified by flash chromatography on silica gel using DCM/MeOH 100/0 to 95/5 to give, the title compound as a white solid (48 mg, 58% yield).

Compound Example 183 was synthesized analogously to Example 181.

### Example 166: (2S)-4-[6-(2-hydroxy-4,6-dimethylphenyl)pyridazin-3-yl]-N-methylmorpholine-2-carboxamide

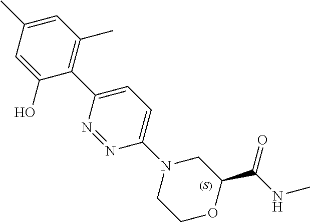

### Int 166.1: (2S)-4-(6-chloropyridazin-3-yl)-N-methyl-morpholine-2-carboxamide

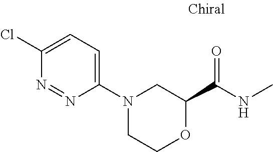 [SMILES: CNC(=O)[C@@H]1CN(c2ccc(Cl)nn2)CCO1]

In a sealed tube, to a stirred solution of (2S)-4-(6-chloropyridazin-3-yl)morpholine-2-carboxylic acid (Int 181.1) (0.821 mmol, 200 mg) in 5 ml of DMF were added, DIPEA (1.23 mmol, 0.214 ml), HATU (0.985 mol, 375 mg) and methylanamine hydrochloride (1.64 mmol, 111 mg). The reaction mixture was stirred at rt for 18 h. DIPEA (0.821 mmol, 0.143 ml) and methanamine hydrochloride (0.821 mmol, 55.4 mg) was added and the mixture was stirred at rt for additional 18 h. The mixture was quenched with water and extracted with DCM. The combined organic layers were washed with an aqueous solution of K₂CO₃ (10%) three times then dried over MgSO₄, filtered, and concentrated under reduced pressure. The residue was purified by flash chromatography on silica gel using DCM/MeOH 100/0 to 97/3 to give the title compound as a white solid (55 mg, 26% yield).

LCMS (Method T): Rt=3.15 min; MS m/z [M+H]+=256.9

### (2S)-4-[6-(2-hydroxy-4,6-dimethylphenyl)pyridazin-3-yl]-N-methylmorpholine-2-carboxamide (Example 166)

In a microwave vial under Ar, a mixture of (2S)-4-(6-chloropyridazin-3-yl)-N-methyl-morpholine-2-carboxamide (0.156 mmol, 40.0 mg), K₂CO₃ (0.296 mmol, 40.9 mg) and (2-hydroxy-4,6-dimethyl-phenyl)boronic acid (0.000222 mol, 36.9 mg,) in 0.8 ml 1,4-dioxane and 0.2 ml of water was degassed under N₂. XPhos (0.00740 mmol, 0.929 mg) and XPhos Pd G2 (0.00740 mmol, 1.53 mg) were added, and the mixture was heated at 90° C. for 18 h. The reaction mixture was then cooling down and concentrated under reduced pressure. The residue was purified by flash chromatography on silica gel using DCM/MeOH 100/0 to 97/3 to give, after lyophilization, the title compound as a white solid (15 mg, 21% yield).

Compound Example 182 was synthesized analogously to Example 166.

### Example 184: (2R)-4-[6-(2-hydroxy-4,6-dimethylphenyl)pyridazin-3-yl]-N,N-dimethylmorpholine-2-carboxamide

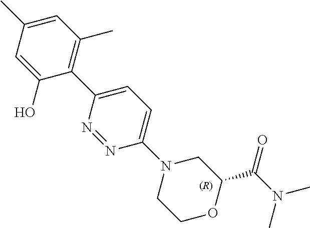

### Int 184.1: (2R)-4-(6-chloropyridazin-3-yl)morpholine-2-carboxylic acid

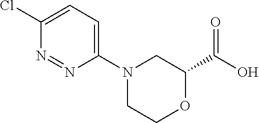 [SMILES: O=C(O)[C@H]1CN(c2ccc(Cl)nn2)CCO1]

In a microwave vial, 3,6-dichloropyridazine (5.51 mmol, 820 mg) and methyl (2R)-morpholine-2-carboxylate hydrochloride (5.51 mmol, 1.00 g) were dissolved in 10 ml of 1-methylpyrrolidin-2-one, and DIPEA (11.0 mmol, 1.42 g) was added. The mixture was heated at 150° C. using one single mode microwave (Biotage Initiator EXP 60) with a power output ranging from 0 to 400 W for 1 h30. After cooling to rt, the mixture was concentrated under reduced pressure.

The residue was purified by reverse phase preparative LC (spherical C18 25 μm, 40 g YMC-ODS-25, dry loading (Celite®), mobile phase gradient 0.2% aq. NH₄+HCO₃—/MeCN from 90:10 to 70:30). The fractions containing the compound were combined and concentrated under reduced pressure. A second purification by reverse phase preparative LC (spherical C18 25 μm, 40 g YMC-ODS-25, dry loading (Celite®), mobile phase gradient 0.2% aq. NH₄+HCO₃—/MeCN 95:5) give the title compound as a white solid (361 mg, 27% yield).

LCMS (Method Q): Rt=1.71 min; MS m/z [M+H]+=244.1;

### Int 184.2: (2R)-4-(6-chloropyridazin-3-yl)-N,N-dimethyl-morpholine-2-carboxamide

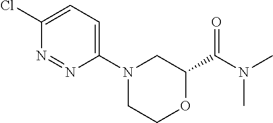 [SMILES: CN(C)C(=O)[C@H]1CN(c2ccc(Cl)nn2)CCO1]

(2R)-4-(6-chloropyridazin-3-yl)morpholine-2-carboxylic acid (0.616 mmol, 150 mg) was dissolved in 2 ml of N,N-dimethylformamide. DIPEA (0.923 mmol, 1 58 μl) and HATU (0.739 mmol, 281 mg) were added, and the mixture was stirred for 10 min. N-dimethylamine 2M in THF (1.23 mmol, 616 μl) was added and the mixture was stirred at rt for 36 h. The mixture was quenched with water and extracted with EtOAc (3×). The combined organic layers were dried over MgSO₄, filtered and concentrated under reduced pressure.

The residue was purified by flash chromatography on silica gel using DCM/MeOH 100/0 to 90/10 to give a white solid. After concentration, the residue was taken up in water and extracted with EtOAc. The organic layer was dried over MgSO₄, filtered and concentrated under reduced pressure to give the title compound as a white solid (25 mg, 15%).

LCMS (Method T): Rt=3.45 min; MS m/z [M+H]+=270.8

### (2R)-4-[6-(2-hydroxy-4,6-dimethylphenyl)pyridazin-3-yl]-N,N-dimethylmorpholine-2-carboxamide (Example 184)

(2R)-4-(6-chloropyridazin-3-yl)-N,N-dimethyl-morpholine-2-carboxamide (0.0923 mmol, 25.0 mg) and (2-hydroxy-4,6-dimethyl-phenyl)boronic acid (0.139 mmol, 23.0 mg) were dissolved in 0.8 ml of 1,4-dioxane and 0.2 ml of water. K₂CO₃ (0.185 mmol, 25.5 mg) was added, and the mixture was purged with N₂. XPhos (0.00462 mmol, 2.20 mg), XPhos Pd G2 (0.00462 mmol, 3.63 mg) were added, the mixture was purged again with N₂ and the mixture was stirred for 18 h at 90° C. Additional (2-hydroxy-4,6-dimethyl-phenyl)boronic acid (0.139 mmol, 23.0 mg), K₂CO₃ (0.185 mmol, 25.5 mg), XPhos (0.00462 mmol, 2.20 mg) and XPhos Pd G2 (0.00462 mmol, 3.63 mg,) were added. The mixture was purged with N₂ and stirred for 3 h at 90° C. After cooling down to rt, water and EtOAc were added. The organic layer was dried over MgSO₄, filtered, and concentrated under reduced pressure. The residue was purified by flash chromatography on silica gel using DCM/MeOH 100/0 to 90/10 to give the title compound as a white solid (7.6 mg, 23% yield).

### Example 196: 3,5-dimethyl-2-[6-(2-oxa-6,9-diazaspiro[4.5]decan-9-yl)pyridazin-3-yl]phenol

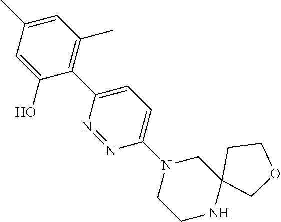 [SMILES: Cc1cc(C)c(-c2ccc(N3CCNC4(CCOC4)C3)nn2)c(O)c1]

### Int 196.1: tert-butyl 9-(6-chloropyridazin-3-yl)-2-oxa-6,9-diazaspiro[4.5]decane-6-carboxylate

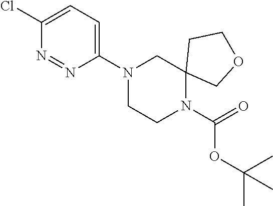 [SMILES: CC(C)(C)OC(=O)N1CCN(c2ccc(Cl)nn2)CC12CCOC2]

To a stirred solution of 3-bromo-6-chloro-pyridazine (0.825 mmol, 160 mg) in 2 ml of 1,4-dioxane at rt were added tert-butyl 2-oxa-6,9-diazaspiro[4.5]decane-6-carboxylate (0.825 mmol, 200 mg) and sodium tert-butoxide (2.48 mmol, 238 mg). The mixture was purged with N₂. RuPhos Pd G3 (0.0825 mmol, 69.0 mg) and RuPhos (0.0825 mmol, 0 38.5 mg) were then added, and the mixture was purged with N₂. The reaction mixture was stirred at 90° C. for 4 h. After cooling down to rt, the mixture was filtered, and the solvent was concentrated under reduced pressure. The residue was purified by flash chromatography on silica gel using DCM/MeOH 100/0 to 95/5 to give the title compound as a colorless oil (175.4 mg, 56% yield).

LCMS (Method R): Rt=1.27 min; MS m/z [M+H]+=355.4;

### Int 196.2: tert-butyl 9-[6-(2-hydroxy-4,6-dimethyl-phenyl)pyridazin-3-yl]-2-oxa-6,9-diazaspiro[4.5]decane-6-carboxylate

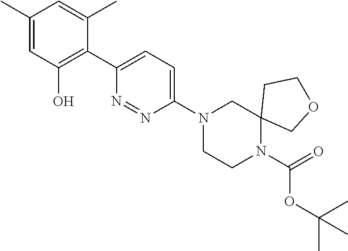 [SMILES: Cc1cc(C)c(-c2ccc(N3CCN(C(=O)OC(C)(C)C)C4(CCOC4)C3)nn2)c(O)c1]

In a microwave vial, under inert atmosphere, was added tert-butyl 9-(6-chloropyridazin-3-yl)-2-oxa-6,9-diazaspiro[4.5]decane-6-carboxylate (0.409 mmol, 156 mg), (2-hydroxy-4,6-dimethyl-phenyl)boronic acid (0.409 mmol, 67.8 mg), XPhos (0.0204 mmol, 8.86 mg) and K₂CO₃ (0.817 mmol, 113 mg) in 2 ml of 1,4-dioxane and 0.4 ml of water. The mixture was purged with N₂. Then, XPhos Pd G2 (0.0204 mmol, 16.1 mg) was added, and the mixture was purged with N₂. The mixture was then heated at 90° C. for 4 hours. The mixture was quenched with water and extracted with DCM (twice). The combined organic layers were dried over MgSO₄, filtered, and concentrated under reduced pressure. The residue was purified by flash chromatography on silica gel using DCM/MeOH 100/0 to 97/03 to give the title compound an off-white solid (137 mg, 69% yield).

LCMS (Method S): Rt=1.32 min; MS m/z [M+H]+=441.4;

### 3,5-dimethyl-2-[6-(2-oxa-6,9-diazaspiro[4.5]decan-9-yl)pyridazin-3-yl]phenol (Example 196)

At 0° C., to a solution of tert-butyl 9-[6-(2-hydroxy-4,6-dimethyl-phenyl)pyridazin-3-yl]-2-oxa-6,9-diazaspiro[4.5]decane-6-carboxylate (0.296 mmol, 131 mg) in 2 ml of DCM was dropwise added TFA (2.96 mmol, 0.220 ml). The reaction mixture was then stirred at rt for 18 hours. After concentration under reduced pressure, the residue was taken up in water, basified with an aqueous solution of K₂CO₃ 10%, extracted with a 90/10 DCM/MeOH mixture, dried over MgSO₄, filtered, and concentrated under reduced pressure. The residue was purified by flash chromatography on silica gel using DCM/MeOH 100/0 to 90/10 to give a colorless oil which was triturated in DIPE, filtered, and dried under reduced pressure to give the title compound as a white solid (51 mg, 39% yield).

### Example 227: 2-[6-(6-ethyl-2-oxa-6,9-diazaspiro[4.5]decan-9-yl)pyridazin-3-yl]-3,5-dimethylphenol

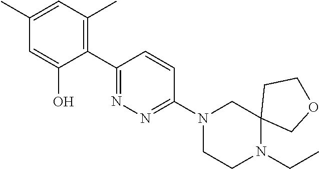 [SMILES: CCN1CCN(c2ccc(-c3c(C)cc(C)cc3O)nn2)CC12CCOC2]

In sealed tube, to a stirred solution of 3,5-dimethyl-2-[6-(2-oxa-6,9-diazaspiro[4.5]decan-9-yl)pyridazin-3-yl]phenol (Example 196, 0.0837 mmol, 28.5 mg) in 1 ml of ethanol at rt were added acetaldehyde (0.126 mmol, 0.007 ml), acetic acid (0.0837 mmol, 0.00479 ml) and NaBH₃CN (0.167 mmol, 10.5 mg). The reaction mixture was stirred at rt for 2 hours. After cooling down to rt, ice-cold water and a saturated solution of NaHCO₃ were slowly added and the mixture was extracted with AcOEt. The combined organic layers were dried over MgSO₄, filtered and concentrated under reduced pressure to give a residue which was purified by reverse phase (Column YMC—Actus Triart Prep C18-S 150*30 mm 5 μm Flow rate 50 ml/min-Focused gradient MeCN/aq·NH₄HCO₃ 0.2% pH=7.9 Focused gradient from 35/65 to 75/25) to give the title compound as an off-white solid (6 mg, 19% yield).

Compound Example 208 was synthesized analogously to Example 227.

### Example 229: 2-[6-[rac-(3aS,6aS)-3,3a,4,5,6,6a-hexahydro-2H-pyrrolo[3,4-b]pyrrol-1-yl]pyridazin-3-yl]-3-methyl-5-(trifluoromethyl)phenol

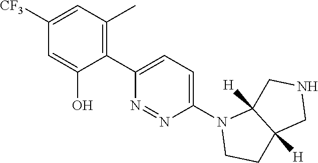 [SMILES: Cc1cc(C(F)(F)F)cc(O)c1-c1ccc(N2CC[C@H]3CNC[C@H]32)nn1]

### Int 229.1: tert-butyl rac-(3aS,6aS)-1-(6-chloropyridazin-3-yl)-2,3,3a,4,6,6a-hexahydropyrrolo[3,4-b]pyrrole-5-carboxylate

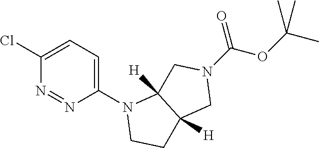 [SMILES: CC(C)(C)OC(=O)N1C[C@@H]2CCN(c3ccc(Cl)nn3)[C@@H]2C1]

In a microwave vial, a mixture of tert-butyl rac-(3aS,6aS)-2,3,3a,4,6,6a-hexahydro-1H-pyrrolo[2,3-c]pyrrole-5-carboxylate (5.03 mmol, 1.07 g), 3,6-dichloropyridazine (5.03 mmol, 0.750 g) and DIPEA (5.03 mmol, 0.862 ml) in 26 ml of NMP was heated at 150° C. using one single mode microwave (Biotage Initiator EXP 60) with a power output ranging from 0 to 400 W for 2 h. NMP was removed with a Genevac. Then, water was added, and the mixture was extracted with DCM (twice). The combined organic layers were dried over MgSO₄, filtered, and concentrated under reduced pressure. The residue was triturated with a mixture of DIPE/MeCN (90/10). The precipitate was filtered off and dried to give the title compound a as pale brown solid (740 mg, 45%).

LCMS (Method T): Rt=5.83 min; MS m/z [M+H]+=324.9;

### Int 229.2: tert-butyl rac-(3aS,6aS)-1-[6-[2-hydroxy-6-methyl-4-(trifluoromethyl)phenyl]pyridazin-3-yl]-2,3,3a,4,6,6a-hexahydropyrrolo[3,4-b]pyrrole-5-carboxylate

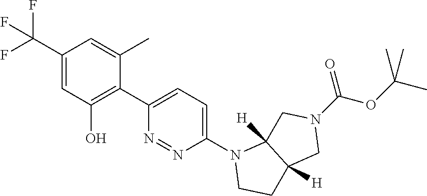 [SMILES: Cc1cc(C(F)(F)F)cc(O)c1-c1ccc(N2CC[C@H]3CN(C(=O)OC(C)(C)C)C[C@H]32)nn1]

In a microwave vial under Ar, a mixture of tert-butyl rac-(3aS,6aS)-1-(6-chloropyridazin-3-yl)-2,3,3a,4,6,6a-hexahydropyrrolo[2,3-c]pyrrole-5-carboxylate (0.308 mmol, 0.100 g), 5-methyl-2-(4,4,5,5-tetramethyl-1,3,2-dioxaborolan-2-yl)-3-(trifluoromethyl)phenol (0.462 mmol, 0.140 g) and K₂CO₃ (0.616 mmol, 0.0851 g) in 3.3 ml of 1,4-dioxane and 0.8 ml of water was degassed under N₂. XPhos Pd G2 (0.0154 mmol, 12.1 mg) and XPhos (0.0154 mmol, 6.68 mg) were added, and the mixture was heated at 90° C. for 3 h. After cooling to rt, the mixture was filtered and washed with DCM. The filtrate was concentrated under reduced pressure. The residue was purified by flash chromatography on silica gel using DCM/MeOH 100/0 to 95/5 to give the title compound (150 mg).

LCMS (Method S): Rt=1.33 min; [M+H]+=465.4;

### 2-[6-[rac-(3aS,6aS)-3,3a,4,5,6,6a-hexahydro-2H-pyrrolo[3,4-b]pyrrol-1-yl]pyridazin-3-yl]-3-methyl-5-(trifluoromethyl)phenol (Example 229)

TFA (3.23 mmol, 0.240 ml) was added dropwise to a solution of tert-butyl rac-(3aS,6aS)-1-[6-[2-hydroxy-6-methyl-4-(trifluoromethyl)phenyl]248yridazine-3-yl]-2,3,3a,4,6,6a-hexahydropyrrolo[2,3-c]pyrrole-5-carboxylate (0.323 mmol, 0.150 g) in 8 ml of DCM at room temperature. The mixture was heated at 30° C. overnight. TFA (3.23 mmol, 0.240 ml) was added dropwise, and the mixture was heated at 30° C. for 4 h. The mixture was concentrated under reduced pressure. The residue was taken up in DCM and washed with an aqueous solution of K₂CO₃ 10%. The organic layers were dried over MgSO₄, filtered and concentrated under reduced pressure. The residue was purified via Reverse phase (Stationary phase: YMC—Actus Triart Prep C18-S 150*30 mm 5 μm Flow rate 50 ml/min-Focused gradient MeCN/aq·NH₄HCO₃ 0.2% pH=7.9 Focused gradient from 25/75 to 55/45) to give, after trituration in DIPE, the title compound as a white solid (23 mg, 20% yield).

### Example 243: 2-[6-[rac-(3aS,6aS)-5-ethyl-2,3,3a,4,6,6a-hexahydropyrrolo[3,4-b]pyrrol-1-yl]pyridazin-3-yl]-3-methyl-5-(trifluoromethyl)phenol

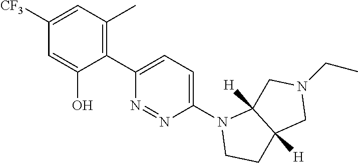 [SMILES: CCN1C[C@@H]2CCN(c3ccc(-c4c(C)cc(C(F)(F)F)cc4O)nn3)[C@@H]2C1]

### Int 243.1: rac-(3aS,6aS)-1-(6-chloropyridazin-3-yl)-3,3a,4,5,6,6a-hexahydro-2H-pyrrolo[3,4-b]pyrrole

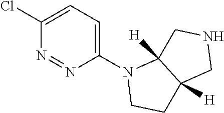 [SMILES: Clc1ccc(N2CC[C@H]3CNC[C@H]32)nn1]

TFA (0.00308 mol, 0.229 ml) was added dropwise to a solution of tert-butyl rac-(3aS,6aS)-1-(6-chloropyridazin-3-yl)-2,3,3a,4,6,6a-hexahydropyrrolo[2,3-c]pyrrole-5-carboxylate (Int 229.1, 0.00160 mmol, 0.520 g) in 15 ml of DCM at rt. The mixture was heated at 30° C. for 8 h. The mixture was concentrated under reduced pressure. The residue was taken up in DCM and washed with an aqueous solution of K₂CO₃ 10%. The organic layer was dried over MgSO₄, filtered, and concentrated under reduced pressure to give 380 mg of the title compound which was used in the next step without any further purification.

LCMS (Method S): Rt=0.65 min; MS m/z [M+H]+=225.1;

### Int 243.2: rac-(3aS,6aS)-1-(6-chloropyridazin-3-yl)-5-ethyl-2,3,3a,4,6,6a-hexahydropyrrolo[3,4-b]pyrrole

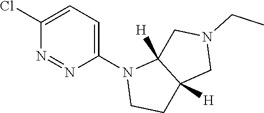 [SMILES: CCN1C[C@@H]2CCN(c3ccc(Cl)nn3)[C@@H]2C1]

To a stirred solution of rac-(3aS,6aS)-1-(6-chloropyridazin-3-yl)-3,3a,4,5,6,6a-hexahydro-2H-pyrrolo[3,4-b]pyrrole (1.09 mmol, 245 mg) in 10 ml of MeCN were added K₂CO₃ (2.18 mmol, 301 mg) and iodoethane (1.09 mmol, 170 mg). The reaction mixture was stirred at rt for 18 h. Water was added to the reaction and the mixture was extracted with DCM. The organics layer was washed with a saturated aqueous solution of NaCl, dried over MgSO₄, filtered, and concentrated under reduced pressure to give 172 mg of the title compound as a white solid, which was used in the next step without any further purification.

LCMS (Method S): Rt=0.75 min; MS m/z [M+H]+=253.1

### 2-[6-[rac-(3aS,6aS)-5-ethyl-2,3,3a,4,6,6a-hexahydropyrrolo[3,4-b]pyrrol-1-yl]pyridazin-3-yl]-3-methyl-5-(trifluoromethyl)phenol (Example 243)

In a microwave vial under Ar, a mixture of rac-(3aS,6aS)-1-(6-chloropyridazin-3-yl)-5-ethyl-2,3,3a,4,6,6a-hexahydropyrrolo[3,4-b]pyrrole (0.673 mmol, 170 mg), K₂CO₃ (1.35 mmol, 186 mg), 3-methyl-2-(4,4,5,5-tetramethyl-1,3,2-dioxaborolan-2-yl)-5-(trifluoromethyl)phenol (0.673 mmol, 203 mg) in 5 ml of 1,4-dioxane and 1.25 ml of water was degassed under N₂ flow. XPhos (0.0336 mmol, 16.0 mg) and XPhos Pd G2 (0.0336 mmol, 26.5 mg) were added, and the mixture was heated at 90° C. for 4 h. After cooling to rt, the reaction mixture was concentrated under reduced pressure. The residue was purified by flash chromatography on silica gel using DCM/(MeOH/NH4OH 10%) 100/0 to 90/10. The fractions containing the compound were combined and concentrated under reduced pressure. The residue was purified by reverse phase chromatography, column YMC—Actus Triart Prep C18-S 150*30 mm 5 μm Flow rate 50 ml/min-Focused gradient MeCN/aq. NH₄HCO₃ 0.2% pH=7.9 Focused gradient from 40/60 to 60/40. The fractions containing the compound were combined and concentrated under reduced pressure and lyophillizated to give the title compound as a white solid (54 mg, 20% yield).

### Example 228: 1-[rac-(3aS,6aS)-1-[6-[2-hydroxy-6-methyl-4-(trifluoromethyl)phenyl]pyridazin-3-yl]-2,3,3a,4,6,6a-hexahydropyrrolo[3,4-b]pyrrol-5-yl]ethanone

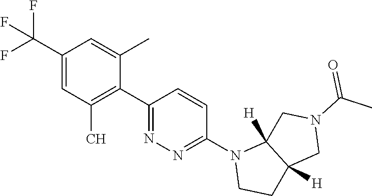 [SMILES: CC(=O)N1C[C@@H]2CCN(c3ccc(-c4c(C)cc(C(F)(F)F)cc4C)nn3)[C@@H]2C1]

### Int 228.1: 1-[rac-(3aS,6aS)-1-(6-chloropyridazin-3-yl)-2,3,3a,4,6,6a-hexahydropyrrolo[3,4-b]pyrrol-5-yl]ethanone

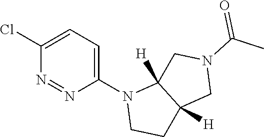 [SMILES: CC(=O)N1C[C@@H]2CCN(c3ccc(Cl)nn3)[C@@H]2C1]

At 0° C., acetyl chloride (0.490 mmol, 0.034 ml) was added dropwise to a solution of (3aS,6aS)-1-(6-chloropyridazin-3-yl)-3,3a,4,5,6,6a-hexahydro-2H-pyrrolo[2,3-c]pyrrole 3 (0.445 mmol, 0.100 g) and DIPEA (0.668 mmol, 0.114 ml) in 6 ml of DCM. The mixture was allowed to warm to rt and stirred for 1 h. The mixture was then quenched with water and extracted with DCM. The organic layer was dried over MgSO₄, filtered, and concentrated under reduced pressure. The residue was purified by flash chromatography on silica gel using DCM/MeOH 100/0 to 95/5 to give the title compound (90 mg).

LCMS (Method S): Rt=0.79 min; MS m/z [M+H]+=267.1;

### 1-[rac-(3aS,6aS)-1-[6-[2-hydroxy-6-methyl-4-(trifluoromethyl)phenyl]pyridazin-3-yl]-2,3,3a,4,6,6a-hexahydropyrrolo[3,4-b]pyrrol-5-yl]ethanone (Example 228)

In a microwave vial under Ar, a mixture of 1-[(3aS1-[(3aS,6aS)-1-(6-chloropyridazin-3-yl)-2,3,3a,4,6,6a-hexahydropyrrolo[2,3-c]pyrrol-5-yl]ethanone (0.292 mmol, 78.0 mg), K₂CO₃ 0.585 mmol, 80.8 mg), 3-methyl-2-(4,4,5,5-tetramethyl-1,3,2-dioxaborolan-2-yl)-5-(trifluoromethyl)phenol (0.292 mmol, 88.3 mg) in 3 ml of 1,4-dioxane and 0.8 ml of water was degassed under N₂ flow. XPhos (1.46e-5 mol, 6.97 mg) and XPhos Pd G2 (1.46e-5 mol, 11.5 mg) were added and the mixture was heated at 90° C. for 1 h. After cooling to rt, the reaction mixture was concentrated under reduced pressure. The residue was purified by flash chromatography on silica gel using DCM/MeOH 100/0 to 90/10 to give, after trituration in DIPE, the title compound (57 mg).

### Example 257: 1-[rac-(4aS,7aS)-4-[6-(2-hydroxy-4,6-dimethylphenyl)pyridazin-3-yl]-2,3,4a,5,7,7a-hexahydropyrrolo[3,4-b][1,4]oxazin-6-yl]ethanone

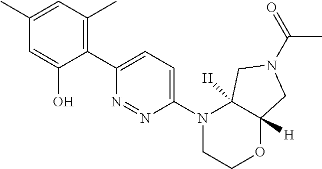 [SMILES: CC(=O)N1C[C@@H]2OCCN(c3ccc(-c4c(C)cc(C)cc4O)nn3)[C@H]2C1]

### Int 257.1: tert-butyl rac-(4aS,7aS)-6-acetyl-2,3,4a,5,7,7a-hexahydropyrrolo[3,4-b][1,4]oxazine-4-carboxylate

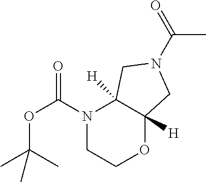 [SMILES: CC(=O)N1C[C@@H]2OCCN(C(=O)OC(C)(C)C)[C@H]2C1]

At 0° C., to a solution of tert-butyl rac-(4aS,7aS)-3,4a,5,6,7,7a-hexahydro-2H-pyrrolo[3,4-b][1,4]oxazine-4-carboxylate (1.18 mmol, 270 mg) in 10 ml of dichloromethane were added triethylamine (1.77 mmol, 247 μl) then acetic anhydride (1.77 mmol, 168 μl). The solution was allowed to warm to rt and stirred for 1 h. DCM was added to the mixture and a 10% aqueous solution of NaHCO₃ was also added. The organic layer was dried over MgSO₄, filtered and concentrated under reduced pressure to give 307 mg of the title compound as a pale yellow oil, which was used in the next step without any further purification.

1H NMR (500 MHz, CDCl₃) δ 4.44-4.14 (m, 1H), 4.08-3.97 (m, 1H), 3.90 (dd, J=11.2, 7.4 Hz, 1H), 3.86-3.65 (m, 2H), 3.63-3.55 (m, 1H), 3.52 (td, J=10.0, 7.5 Hz, 1H), 3.41-3.23 (m, 1H), 3.14 (dd, J=11.9, 9.3 Hz, 1H), 3.07-2.93 (m, 1H), 2.04 (d, J=13.7 Hz, 3H), 1.48 (s, 11H).

### Int 257.2: 1-[rac-(4aS,7aS)-3,4,4a,5,7,7a-hexahydro-2H-pyrrolo[3,4-b][1,4]oxazin-6-yl]ethanone; 2,2,2-trifluoroacetate

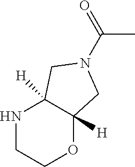

At 0° C., to a solution of tert-butyl (4aS,7aS)-6-acetyl-2,3,4a,5,7,7a-hexahydropyrrolo[3,4-b][1,4]oxazine-4-carboxylate (1.11 mmol, 300 mg) in 10 m of dichloromethane was slowly added TFA (11.1 mmol, 824 μl). The solution was allowed to warm to rt and stirred for 1 h. The solution was concentrated under reduced pressure, then co-evaporated with toluene to give 530 mg of the title compound as a colorless residue, which was used in the next step without any further purification.

1H NMR (400 MHz, CDCl₃) δ (ppm): 4.34-3.85 (m, 5H), 3.73-3.14 (m, 5H), 2.13 (s, 3H).

### Int 257.3: 1-[rac-(4aS,7aS)-4-(6-chloropyridazin-3-yl)-2,3,4a,5,7,7a-hexahydropyrrolo [3,4-b][1,4]oxazin-6-yl]ethanone

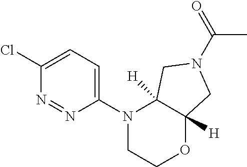 [SMILES: CC(=O)N1C[C@@H]2OCCN(c3ccc(Cl)nn3)[C@H]2C1]

In a sealed tube, a mixture of 3-bromo-6-chloro-pyridazine (1.17 mmol, 227 mg), sodium tertbutoxide (3.17 mmol, 304 mg), RuPhos Pd G3 (0.0528 mmol, 44.1 mg) and RuPhos (0.106 mol, 49.3 mg) was purged with N₂. A solution of 1-[(4aS,7aS)-3,4,4a,5,7,7a-hexahydro-2H-pyrrolo[3,4-b][1,4]oxazin-4-ium-6-yl]ethanone; 2,2,2-trifluoroacetate (1.06 mmol, 300 mg) in 6 ml of 1,4-dioxane was added and the mixture was purged again with N₂ then heated at 100° C. for 2 h. After cooling to rt, the mixture was diluted with EtOAc, then water was added. The aqueous layer was extracted with EtOAc. The combined organic layers were dried over MgSO₄, filtered, and concentrated under reduced pressure. The residue was purified by flash chromatography on silica gel using DCM/MeOH 100/0 to 95/5, then isocratic 95/5 to give 149 mg of the title compound.

LCMS (Method S): Rt=0.81 min; MS m/z [M+H]+=283.1;

### 1-[rac-(4aS,7aS)-4-[6-(2-hydroxy-4,6-dimethylphenyl)pyridazin-3-yl]-2,3,4a,5,7,7a-hexahydropyrrolo[3,4-b][1,4]oxazin-6-yl]ethanone (Example 257)

In a sealed tube, a mixture of (2-hydroxy-4,6-dimethyl-phenyl)boronic acid (0.621 mmol, 103 mg), XPhos Pd G2 (0.0414 mmol, 32.6 mg), XPhos (0.0828 mmol, 39.5 mg) and K₂CO₃ (0.828 mmol, 114 mg) was purged with N₂. 1-[(4aS,7aS)-4-(6-chloropyridazin-3-yl)-2,3,4a,5,7,7a-hexahydropyrrolo[3,4-b][1,4]oxazin-6-yl]ethanone (0.414 mmol, 117 mg) in 5.8 ml of 1,4-dioxane and 1.2 ml of water was added, the mixture was purged again with N₂ then heated at 90° C. for 3 h. After cooling to rt, the mixture was diluted with EtOAc and water was added. The aqueous layer was extracted with EtOAc. The combined organic layers were dried over MgSO₄, filtered, and concentrated under reduced pressure. The residue was purified by flash chromatography on silica gel using DCM/MeOH 100/0 to 95/5, then isocratic 95/5.

The fractions containing the compound were combined and concentrated under reduced pressure. The residue was purified by reverse phase preparative LC (Column YMC—Actus Triart Prep C18-S 150*30 mm 5 μm Flow rate 50 ml/min-Focused gradient MeCN/aq·NH₄HCO₃ 0.2% pH=7.9 Focused gradient from 25/75 to 55/45) to give, after lyophilization, 49.0 mg of the title compound as a white fluffy solid.

### Example 265: 2-[6-[rac-(3aS,6aS)-5-methyl-2,3,3a,4,6,6a-hexahydropyrrolo[3,4-b]pyrrol-1-yl]pyridazin-3-yl]-3-methyl-5-(trifluoromethyl)phenol

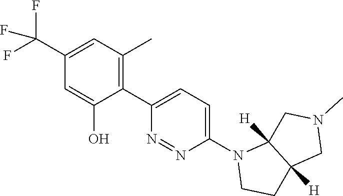 [SMILES: Cc1cc(C(F)(F)F)cc(O)c1-c1ccc(N2CC[C@H]3CN(C)C[C@H]32)nn1]

### Int 265.1: tert-butyl rac-(3aS,6aS)-1-(6-chloropyridazin-3-yl)-2,3,3a,4,6,6a-hexahydropyrrolo[3,4-b]pyrrole-5-carboxylate

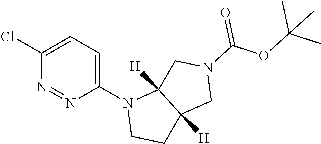 [SMILES: CC(C)(C)OC(=O)N1C[C@@H]2CCN(c3ccc(Cl)nn3)[C@@H]2C1]

In a microwave vial, a mixture of tert-butyl rac-(3aS,6aS)-2,3,3a,4,6,6a-hexahydro-1H-pyrrolo[2,3-c]pyrrole-5-carboxylate (5.03 mmol, 1.07 g), 3,6-dichloropyridazine (5.03 mmol, 0.750 g) and DIPEA (5.03 mmol, 0.862 ml) in 26 ml of NMP was heated at 150° C. using one single mode microwave (Biotage Initiator EXP 60) with a power output ranging from 0 to 400 W for 2 h. NMP was removed with a Genevac. Water was added to the residue and the mixture was extracted twice with DCM. The combined organic layers were dried over MgSO₄, filtered, and concentrated under reduced pressure. The residue was triturated with a mixture of DIPE/MeCN (90/10). The precipitate was filtered and dried to give 740 mg of the title compound as pale brown solid.

LCMS (Method T): Rt=5.83 min; MS m/z [M+H]+=324.9;

### Int 265.2: rac-(3aS,6aS)-1-(6-chloropyridazin-3-yl)-3,3a,4,5,6,6a-hexahydro-2H-pyrrolo[3,4-b]pyrrole

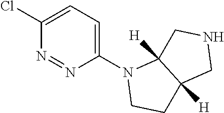 [SMILES: Clc1ccc(N2CC[C@H]3CNC[C@H]32)nn1]

TFA (0.00308 mol, 0.229 ml) was added dropwise to a solution of tert-butyl rac-(3aS,6aS)-1-(6-chloropyridazin-3-yl)-2,3,3a,4,6,6a-hexahydropyrrolo[2,3-c]pyrrole-5-carboxylate (0.00160 mmol, 0.520 g) in 15 ml of DCM at room temperature. The mixture was heated at 30° C. for 8 h. After cooling to rt, the mixture was concentrated under reduced pressure. The residue was taken up in DCM and washed with an aqueous solution of K₂CO₃ 10%. The organic layer was dried over MgSO₄, filtered, and concentrated under reduced pressure to give 380 mg of the title compound, which was used without any further purification.

LCMS (Method S): Rt=0.65 min; MS m/z [M+H]+=225.1;

### Int 265.3: rac-(3aS,6aS)-1-(6-chloropyridazin-3-yl)-5-methyl-2,3,3a,4,6,6a-hexahydropyrrolo[3,4-b]pyrrole

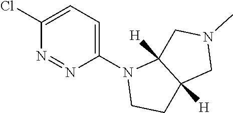 [SMILES: CN1C[C@@H]2CCN(c3ccc(Cl)nn3)[C@@H]2C1]

Formaldehyde (37% in water) (9.79 mmol, 0.771 ml) was added to a solution of rac-(3aS,6aS)-1-(6-chloropyridazin-3-yl)-3,3a,4,5,6,6a-hexahydro-2H-pyrrolo[2,3-c]pyrrole (1.96 mmol, 440 mg) in 10 ml of methanol and 10 ml of THF, and stirred at rt. NaBH₃CN (1.96 mmol, 123 mg) was added and the resulting mixture was stirred at rt for 2 h. The reaction mixture was poured on ice and extracted with DCM. The organic layer was dried over MgSO₄, filtered, and concentrated under reduced pressure. The residue was purified by flash chromatography on silica gel using DCM/(MeOH/NH₄OH 10%) 100/0 to 90/10 to give 270 mg of the title compound (57% yield).

LCMS (Method R): Rt=0.99 min; MS m/z [M+H]+=239.2;

### 2-[6-[rac-(3aS,6aS)-5-methyl-2,3,3a,4,6,6a-hexahydropyrrolo[3,4-b]pyrrol-1-yl]pyridazin-3-yl]-3-methyl-5-(trifluoromethyl)phenol (Example 265)

In a microwave vial under Ar, a mixture of rac-(3aS,6aS)-1-(6-chloropyridazin-3-yl)-5-methyl-2,3,3a,4,6,6a-hexahydropyrrolo[2,3-c]pyrrole (1.01 mmol, 240 mg), K₂CO₃ (2.01 mmol, 278 mg) and 3-methyl-2-(4,4,5,5-tetramethyl-1,3,2-dioxaborolan-2-yl)-5-(trifluoromethyl)phenol (1.51 mmol, 456 mg) in 0.8 ml of 1,4-dioxane and 0.2 ml of water was degassed under N₂ flow. XPhos (0.0503 mmol, 24.0 mg) and XPhos Pd G2 (0.0503 mmol, 39.6 mg) were added, and the mixture was heated at 80° C. for 3 h. The reaction mixture was cooled to rt then concentrated under reduced pressure. The residue was purified by flash chromatography on silica gel using DCM/(MeOH/NH₄OH 10%) 100/0 to 80/20. The fractions containing the compound were combined and concentrated under reduced pressure. The residue was purified by reverse phase preparative LC (Column YMC—Actus Triart Prep C18-S 150*30 mm 5 μm Flow rate 50 ml/min-Focused gradient MeCN/aq·NH₄HCO₃ 0.2% pH=7.9 Focused gradient from 45/55 to 65/35) to give 181 mg of the title compound.

### Examples 270 and 271: 2-[6-[(3aR,6aR)-5-methyl-2,3,3a,4,6,6a-hexahydropyrrolo[3,4-b]pyrrol-1-yl]pyridazin-3-yl]-3-methyl-5-(trifluoromethyl)phenol and 2-[6-[(3aS,6aS)-5-methyl-2,3,3a,4,6,6a-hexahydropyrrolo[3,4-b]pyrrol-1-yl]pyridazin-3-yl]-3-methyl-5-(trifluoromethyl)phenol

The 2 enantiomers of compound 2-[6-[(3aS,6aS)-5-methyl-2,3,3a,4,6,6a-hexahydropyrrolo[3,4-b]pyrrol-1-yl]pyridazin-3-yl]-3-methyl-5-(trifluoromethyl)phenol (Example 265, 360 mg) were separated by preparative chiral SFC chromatography (Stationary phase: Chiralpak® AS-H 20×250 mm, Mobile phase: CO₂/(iPrOH+0.3% iPrNH₂) 85/15 to give both enantiomers, first eluting Rt 2.64 min, example 270 (66 mg) and second eluting Rt 4.11 min, example 271 (74 mg). The absolute configurations cannot be assigned at this stage.

### Example 245: 5-ethyl-2-[6-[(2S)-2-(hydroxymethyl)morpholin-4-yl]pyridazin-3-yl]-3-methylphenol

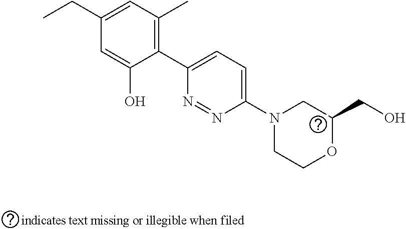 [SMILES: CCc1cc(C)c(-c2ccc(N3CCO[C@H](CO)C3)nn2)c(O)c1]

### Int 245.1: 5-ethyl-2-iodo-3-methyl-phenol

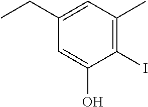

Under Ar, at 0° C., to a stirred solution of 3-ethyl-5-methyl-phenol (1.00 g, 7.34 mmol) in 60 ml of toluene was added portionwise NaH (60% in mineral oil) (0.587 g, 14.7 mmol). The reaction mixture was stirred at 0° C. for 30 min, then was added portionwise 12 (1.86 g, 7.34 mmol). The reaction mixture was stirred at 0° C. for 20 min, then poured into cold water, and extracted with EtOAc. The organic layer was washed with an aqueous solution of Na₂S₂O₃. The organic layer was dried over MgSO₄, filtered, and concentrated under reduced pressure. The residue was purified by flash chromatography on silica gel using heptane/EtOAc (100/0 to 80/20). The fractions containing the compound were combined and concentrated under reduced pressure. The residue was purified by SFC Chiralpak® AD-H 20×250 mm, Chiralpak® IG 30×250 mm, Mobile phase: CO₂/(iPrOH+0.3% iPrNH2) 85/15 to give 445 mg of the title compound.

1H NMR (500 MHz, DMSO-d6) δ (ppm): 10.05 (s, 1H), 6.63 (s, 1H), 6.53 (s, 1H), 2.45 (q, J=7.6 Hz, 2H), 2.31 (s, 3H), 1.12 (t, J=7.5 Hz, 3H).

LCMS (Method S): Rt=1.33 min; MS m/z [M+H]+=261.0;

### Int 245.2: 5-ethyl-3-methyl-2-(4,4,5,5-tetramethyl-1,3,2-dioxaborolan-2-yl)phenol

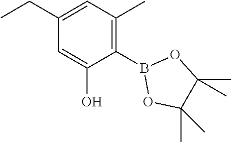 [SMILES: CCc1cc(C)c(B2OC(C)(C)C(C)(C)O2)c(O)c1]

In a sealed tube, NEt₃ (0.819 mL, 5.88 mmol) was slowly added to a solution of 5-ethyl-2-iodo-3-methyl-phenol (220 mg, 0.839 mmol) and HBpin (537 mg, 4.20 mmol) in 12 ml of 1,4-dioxane. The solution was purged with N₂. Pd(OAc)₂ (22.6 mg, 0.101 mmol) and CyJohnPhos (0.7 mg, 0.185 mmol) were added, the mixture was purged again with N₂, then heated at 80° C. for 18 h. After cooling to rt, water was added. The aqueous layer was separated and extracted with EtOAc. The combined organic layers were dried over MgSO₄, filtered and concentrated under reduced pressure. The residue was purified by flash chromatography on silica gel using heptane/EtOAc (100/0 to 90/10) to give 164 mg of the title compound.

1H NMR (500 MHz, DMSO-d6) δ (ppm): δ 8.72 (s, 1H), 6.46 (s, 1H), 6.41 (s, 1H), 2.46 (q, J=7.8 Hz, 2H), 2.26 (s, 3H), 1.30 (s, 12H), 1.12 (t, J=7.6 Hz, 3H)+ undefined impurities

### 5-ethyl-2-[6-[(2S)-2-(hydroxymethyl)morpholin-4-yl]pyridazin-3-yl]-3-methylphenol

### Example 245

In a microwave vial under Ar, a mixture of [(2S)-4-(6-chloropyridazin-3-yl)morpholin-2-yl]methanol (88.0 mg, 0.383 mmol), K₂CO₃ (106 mg, 0.766 mmol), 5-ethyl-3-methyl-2-(4,4,5,5-tetramethyl-1,3,2-dioxaborolan-2-yl)phenol (140 mg, 0.534 mol) in 3 ml of 1,4-dioxane and 0.75 ml of water was degassed under N₂. XPhos (9.34 mg, 0.0196 mmol) and XPhos Pd G2 (15.1 mg, 0.0192 mmol) were added, and the mixture was heated at 80° C. for 6 h. After cooling to rt, the mixture was filtrated over Celite® and concentrated under reduced pressure. The residue was purified by flash chromatography on silica gel using DCM/MeOH (100/0 to 95/5). The fractions containing the compound were combined and concentrated under reduced pressure. The residue was purified by reverse phase preparative LC Column YMC—Actus Triart Prep C18-S 150*30 mm 5 μm Flow rate 50 ml/min-Focused gradient MeCN/aq·NH₄HCO₃ 0.2% pH=7.9 (Focused gradient from 25/75 to 65/35) to give, after lyophilisation, 22 mg of the title compound.

### Example 246: 3-ethyl-2-[6-[(2S)-2-(hydroxymethyl)morpholin-4-yl]pyridazin-3-yl]-5-methylphenol

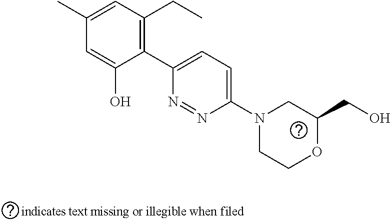 [SMILES: CCc1cc(C)cc(O)c1-c1ccc(N2CCO[C@H](CO)C2)nn1]

### Int 246.1: 3-ethyl-2-iodo-5-methyl-phenol

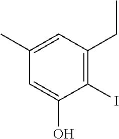

Under Ar, at 0° C., to a stirred solution of 3-ethyl-5-methyl-phenol (1.00 g, 7.34 mmol) in 60 ml of toluene was added portionwise NaH (60% in mineral oil) (0.587 g, 14.7 mmol). The reaction mixture was stirred at 0° C. for 30 min, then was added portionwise 12 (1.86 g, 7.34 mmol). The reaction mixture was stirred at 0° C. for 20 min, then poured into cold water, and extracted with EtOAc. The organic layer was washed with an aqueous solution of Na₂S203. The organic layer was dried over MgSO₄, filtered and concentrated under reduced pressure. The residue was purified by flash chromatography on silica gel using heptane/EtOAc (100/0 to 80/20). The fractions containing the compound were combined and concentrated under reduced pressure. The residue was purified by SFC Chiralpak® AD-H 20×250 mm, Chiralpak® IG 30×250 mm, Mobile phase: CO₂/(iPrOH+0.3% iPrNH₂) 85/15 to give 181 mg of the title compound.

1H NMR (500 MHz, DMSO-d6) δ (ppm): 10.03 (s, 1H), 6.57 (s, 1H), 6.52 (s, 1H), 2.62 (q, J=7.5 Hz, 2H), 2.17 (s, 3H), 1.10 (t, J=7.5 Hz, 3H).

LCMS (Method S): Rt=1.32 min; MS m/z [M+H]+=261.0;

### Int 246.2: 3-ethyl-5-methyl-2-(4,4,5,5-tetramethyl-1,3,2-dioxaborolan-2-yl)phenol

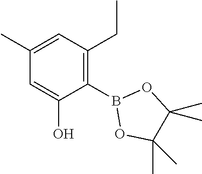

In a sealed tube, NEt₃ (0.670 mL, 4.81 mmol) was slowly added to a solution of 3-ethyl-2-iodo-5-methyl-phenol (180 mg, 0.687 mol) and HBpin (0.498 mL, 3.43 mmol) in 8 ml of 1,4-dioxane. The solution was purged with N₂. Pd(OAc)₂ (18.5 mg, 0.0824 mmol) and CyJohnPhos (53.0 mg, 0.151 mmol) were added, the mixture was purged again with N₂ then heated at 80° C. for 18 h. After cooling to rt, water was added. The aqueous layer was separated and extracted with EtOAc. The combined organic layers were dried over MgSO₄, filtered, and concentrated under reduced pressure. The residue was purified by flash chromatography on silica gel using heptane/EtOAc (100/0 to 90/10) to give 93 mg of the title compound.

1H NMR (500 MHz, DMSO-d6) δ (ppm): 8.81 (s, 1H), 6.44 (s, 1H), 6.40-6.35 (m, 1H), 2.59-2.50 (m, 2H), 2.17 (s, 3H), 1.29 (s, 12H), 1.08 (t, J=7.5 Hz, 3H)+ undefined impurities.

### 3-ethyl-2-[6-[(2S)-2-(hydroxymethyl)morpholin-4-yl]pyridazin-3-yl]-5-methylphenol (Example 246)

In a microwave vial under Ar, a mixture of [(2S)-4-(6-chloropyridazin-3-yl)morpholin-2-yl]methanol (55.0 mg, 2.39 mmol), K₂CO₃ (66.2 mg, 0.479 mmol), 3-ethyl-5-methyl-2-(4,4,5,5-tetramethyl-1,3,2-dioxaborolan-2-yl)phenol (94.2 mg, 0.359 mmol) in 2 ml of 1,4-dioxane and 0.5 ml of water was degassed under N₂ flow. XPhos (5.71 mg, 0.012 mmol) and XPhos Pd G2 (9.42 mg, 0.012 mmol) were added, and the mixture was heated at 80° C. for 6 h. After cooling to rt, the mixture was filtrated over Celite® and concentrated under reduced pressure. The residue was purified by flash chromatography on silica gel using DCM/MeOH (100/0 to 95/5). The fractions containing the compound were combined and concentrated under reduced pressure. The residue was purified by reverse phase preparative LC Column YMC—Actus Triart Prep C18-S 150*30 mm 5 μm Flow rate 50 ml/min-Focused gradient MeCN/aq·NH₄HCO₃ 0.2% pH=7.9 (Focused gradient from 25/75 to 65/35) to give, after lyophilisation, 14 mg of the title compound.

### Examples 260 and 261: 3-methyl-2-[6-[(4aR,8aS)-6-methyl-3,4a,5,7,8,8a-hexahydro-2H-pyrido[4,3-b][1,4]oxazin-4-yl]pyridazin-3-yl]-5-(trifluoromethyl)phenol and 3-methyl-2-[6-[(4aS,8aR)-6-methyl-3,4a,5,7,8,8a-hexahydro-2H-pyrido[4,3-b][1,4]oxazin-4-yl]pyridazin-3-yl]-5-(trifluoromethyl)phenol

The 2 enantiomers of compound 2-[6-[rac-(4aR,8aS)-6-methyl-3,4a,5,7,8,8a-hexahydro-2H-pyrido[4,3-b][1,4]oxazin-4-yl]pyridazin-3-yl]-3-methyl-5-(trifluoromethyl)phenol (Example 226, 101 mg) were separated by Supercritical Fluidic Chromatography (SFC—Column: Daicel Chiralpak® AD-H, 250×30 mm I.D., 5 μm particle size; Mobile phase: C₀₂/Ethanol (+0.1% Triethylamine) 20%; flow rate: 200 mL/min; outlet pressure: 100 bar; 2 injections; UV detection: 265 nm) to provide 3-methyl-2-[6-[(4aR,8aS)-6-methyl-3,4a,5,7,8,8a-hexahydro-2H-pyrido[4,3-b][1,4]oxazin-4-yl]pyridazin-3-yl]-5-(trifluoromethyl)phenol, first eluting enantiomer at Rt=4.3 min (22 mg, Example 260, Enantiomer 1) as a white solid, and 3-methyl-2-[6-[(4aS,8aR)-6-methyl-3,4a,5,7,8,8a-hexahydro-2H-pyrido[4,3-b][1,4]oxazin-4-yl]pyridazin-3-yl]-5-(trifluoromethyl)phenol, second eluting enantiomer at Rt=6 min (20 mg, Example 261, Enantiomer 2), as a white solid. The absolute configurations cannot be assigned at this stage.

Compounds Examples 310, 311 were obtained by preparative chiral SFC separation analogously to Example 260 and 261. The absolute configurations were assigned a posteriori (see Chiral synthesis of example 311).

### Example 260: 2-[6-[(4aS,8aR)-6-methyl-3,4a,5,7,8,8a-hexahydro-2H-pyrido[4,3-b][1,4]oxazin-4-yl]pyridazin-3-yl]-3-methyl-5-(trifluoromethyl)phenol

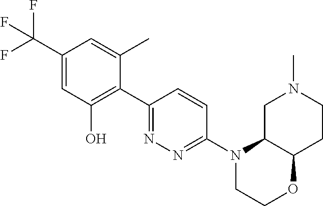 [SMILES: Cc1cc(C(F)(F)F)cc(O)c1-c1ccc(N2CCO[C@@H]3CCN(C)C[C@@H]32)nn1]

To a solution of 3-methyl-2-(4,4,5,5-tetramethyl-1,3,2-dioxaborolan-2-yl)-5-(trifluoromethyl)phenol (2.23 mmol, 674 mg) and (4aS,8aR)-4-(6-chloropyridazin-3-yl)-6-methyl-3,4a,5,7,8,8a-hexahydro-2H-pyrido[4,3-b][1,4]oxazine (Int 321.3-1.49 mmol, 400 mg) in 60 ml of 1,4-dioxane and 10 ml of water was added a 2N sodium carbonate aqueous solution (4.47 mmol, 2.23 ml). Argon was bubbling through the solution for 10 min then tetrakis(triphenylphosphine)palladium(0) (0.149 mmol, 172 mg) was added and the solution was stirred at 100° C. for 2 h. After cooling down to rt, the mixture was concentrated under reduced pressure and the residue was diluted with 100 ml of DCM and 100 ml of a saturated aqueous sodium bicarbonate solution. The aqueous layer was extracted with 2×50 ml of DCM and the combined organic layers were dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 90/4.9/4.9/0.2) to give, after trituation in diethylether, the title compound as a white solid (390 mg, 64% yield).

### Example 127: 5-chloro-2-[6-[(3S)-3-(hydroxymethyl)pyrrolidin-1-yl]pyridazin-3-yl]-3-methyl-phenol

### Int 127.1: [(3S)-1-(6-chloropyridazin-3-yl)pyrrolidin-3-yl]methanol

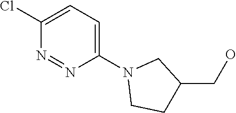 [SMILES: OCC1CCN(c2ccc(Cl)nn2)C1]

Under Ar, to a solution of 3,6-dichloropyridazine (250 mg, 1.6781 mmol) and triethylamine (515 μl, 3.6918 mmol) in 20 ml of n-butanol was added [(3S)-pyrrolidin-3-yl]methanol (187 mg, 1459 mmol) and the mixture was heated at 120° C. for 4 h. After cooling to rt, the reaction mixture was diluted with AcOEt, washed with water and brine, dried over MgSO₄ and concentrated under reduced pressure. The residue was crystallized in diethyl ether and filtrated off to give the title compound as a beige solid (m=190 mg, 52% yield)

LCMS (Method L): Rt=0.34 min; MS m/z [M+H]+=213;

### Int 127.2: [(3S)-1-[6-(4-chloro-2-methoxy-6-methyl-phenyl)pyridazin-3-yl]pyrrolidin-3-yl]methanol

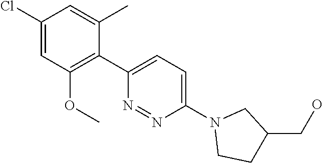 [SMILES: COc1cc(Cl)cc(C)c1-c1ccc(N2CCC(CO)C2)nn1]

In a microwave vial, under Ar, to a solution of [(3S)-1-(6-chloropyridazin-3-yl)pyrrolidin-3-yl]methanol (100 mg, 0.468 mmol), 2-(4-chloro-2-methoxy-6-methyl-phenyl)-4,4,5,5-tetramethyl-1,3,2-dioxaborolane (172 mg, 0.6084 mmol), carbonate sodium (117 mg, 1.4041 mmol) in 6.23 ml of 1,4-dioxane and 1.56 ml of Water was add tetrakis(triphenylphosphine)palladium(0) (38 mg, 0.0328 mmol). The mixture was heated at 120° C. under microwave irradiation for 3 h. After cooling to rt, the reaction mixture was diluted with AcOEt, washed with water and brine, dried over MgSO₄, concentrated under reduced pressure. The residue was purified by flash chromatography SiO₂ DCM/MeOH (from 100/0 to 95/5) to give the title compound as an amorphous solid (76 mg, 48% yield).

LCMS (Method L) Rt=0.54 min MS m/z [M+H]+=334;

### 5-chloro-2-[6-[(3S)-3-(hydroxymethyl)pyrrolidin-1-yl]pyridazin-3-yl]-3-methyl-phenol (Example 127)

At −5° C., to a solution of [(3S)-1-[6-(4-chloro-2-methoxy-6-methyl-phenyl)pyridazin-3-yl]pyrrolidin-3-yl]methanol (76 mg, 0.2277 mmol) in 10 ml of dichloromethane was slowly added Tribromoboron (455 μl, 0.4553 mmol, 1 mol/l in hexane) and the reaction mixture was stirred at −5° C. for 1 h15. Additional Tribromoboron (200 μl) was added, and the mixture was stirred for 1 h15 more at −5° C. Then at −5° C. few drops of methanol were slowly added, and the mixture was basified with aqueous NaOH 5N, stirred 5 mn, then acidified with an aqueous solution of aqueous SO₂ 6%. The mixture was diluted with water and extracted with DCM. The organic layer was washed with brine, dried over Mg₂SO₄, filtered, and concentrated under reduce pressure. The residue was crystallized in diethyl ether, filtered off, and purified by reverse phase chromatography (H₂O/CH₃CN) (C18, water/MeCN, gradient 95/5 to 5/95) to give the title compound as a beige solid (7 mg, 10% yield).

### Example 130: (4S)-1-[6-(4-chloro-2-hydroxy-6-methyl-phenyl)pyridazin-3-yl]-4-hydroxy-pyrrolidin-2-one

### Int 130.1: (4S)-1-(6-chloropyridazin-3-yl)-4-hydroxy-pyrrolidin-2-one

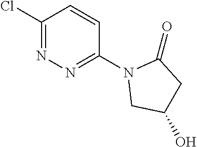 [SMILES: O=C1C[C@H](O)CN1c1ccc(Cl)nn1]

In a microwave vial, under Ar, to a solution of 3,6-dichloropyridazine (2.25 mmol, 335 mg) and (S)-4-hydroxy-2-pyrrolidinone (2.56 mmol, 259 mg) in 5 ml of 1,4-dioxane was added potassium phosphate tribasic (4.27 mmol, 907 mg), 1-1′-bis(diphenylphosphino)ferrocene (0.26 mmol, 142 mg), palladium acetate (0.09 mmol, 19 mg). The vial was sealed, and the mixture was heated at 110° C. under microwave irradiation for 1.5 h. After cooling to rt, the reaction mixture was diluted with EtOAc and CH₂Cl₂ then filtered, the solid part was washed with EtOAc and CH₂Cl₂, and the filtrate was concentrated under reduced pressure. The residue was purified by flash chromatography on silica gel using DCM/EtOAc (from 100/0 to 50/50) to give the title compound as a brown solid (145 mg, 32% yield).

LCMS (Method M): Rt=0.76 min; MS m/z [M+H]+ 214;

### (4S)-1-[6-(4-chloro-2-hydroxy-6-methyl-phenyl)pyridazin-3-yl]-4-hydroxy-pyrrolidin-2-one (Example 130)

In a microwave vial, under Ar, to a solution of (4S)-1-(6-chloropyridazin-3-yl)-4-hydroxy-pyrrolidin-2-one (0.35 mmol, 75 mg) and (4-chloro-2-hydroxy-6-methylphenyl)boronic acid (0.37 mmol, 69 mg) in 1.8 ml of 1,2-dimethoxyethane were added sodium carbonate (0.88 mmol, 93 mg) and 0.45 ml of water. The solution was purged with Ar, then Pd(PPh₃)₄ (0.02 mmol, 20 mg) was added. The vial was sealed, and the mixture was heated at 100° C. under microwave irradiation for 1 h. The reaction mixture was diluted with EtOAc and water and filtered. The organic layer was separated, washed with water, dried over Na₂SO₄, filtered and concentrated under reduced pressure. The residue was purified by flash chromatography on silica gel using DCM/MeOH/ACN (from 100/0/0 to 93/3.5/3.5) to give the title compound as a white solid (27 mg, 24% yield).

### Example 162:4-[6-(2-hydroxy-4,6-dimethyl-phenyl)pyridazin-3-yl]-2,3-dihydro-1,4-benzoxazin-6-ol

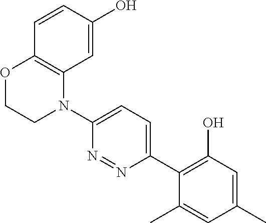 [SMILES: Cc1cc(C)c(-c2ccc(N3CCOc4ccc(O)cc43)nn2)c(O)c1]

### Int 162.1: 4-(6-chloropyridazin-3-yl)-2,3-dihydro-1,4-benzoxazin-6-ol

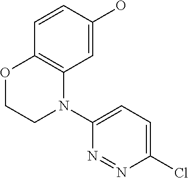 [SMILES: Oc1ccc2c(c1)N(c1ccc(Cl)nn1)CCO2]

In a microwave vial, under Ar, a suspension of 3,6-dichloropyridazine (5.97 mmol, 889 mg), 3,4-dihydro-2H-benzo[b][1,4]oxazin-6-ol hydrochloride (0.75 mmol, 140 mg) and diisopropylethylamine (1.64 mmol, 0.28 ml) in 1 ml of n-butanol was heated at 150° C. under microwave irradiation for 7.5 h. After addition of DCM, the organic layer was washed with water, filtered on hydrophobic column and the filtrate was concentrated under reduced pressure. The residue was purified by flash chromatography on silica gel using DCM/MeOH/ACN (from 100/0/0 to 94/3/3) to give the title compound as a brown solid (63 mg, 32% yield).

LCMS (Method U): Rt=1.17 min; MS m/z [M+H]+=264;

### 4-[6-(2-hydroxy-4,6-dimethyl-phenyl)pyridazin-3-yl]-2,3-dihydro-1,4-benzoxazin-6-ol (Example 162)

In a microwave vial, under Ar, to a solution of 4-(6-chloropyridazin-3-yl)-2,3-dihydro-1,4-benzoxazin-6-ol (0.24 mmol, 62 mg) and (2-hydroxy-4,6-dimethylphenyl)boronic acid (0.26 mmol, 43 mg) in 1.65 ml of 1,2-dimethoxyethane were added sodium carbonate (0.59 mmol, 62 mg) and 0.4 ml of water. The solution was purged with Ar, then Pd(PPh₃)₄ (0.02 mmol, 19 mg) was added. The vial was sealed, and the mixture was heated at 100° C. under microwave irradiation for 1 h. After cooling to rt, the reaction mixture was diluted with EtOAc and filtered. The organic layer was washed with water, dried over Na₂SO₄, filtered and concentrated under reduced pressure. The residue was purified by flash chromatography on silica gel using DCM/MeOH/ACN (from 100/0/0 to 96/2/2) to give a brown solid (45 mg). This one was further purified by reverse phase chromatography (Column XSelect CSH C18 5 μm OBD from Waters, 250×50 mm, Flow rate 80 ml/min, eluent: acetonitrile/water+0.1% formic acid (from 34/66 to 100/0), to give the title compound as an off white solid (30 mg, 37% yield).

Compounds Examples 193, 215 and 362 were synthesized analogously to Example 162.

### Example 123: 2-[6-[(3R)-3-(aminomethyl)morpholin-4-yl]pyridazin-3-yl]-3,5-dimethyl-phenol; 2,2,2-trifluoroacetic acid

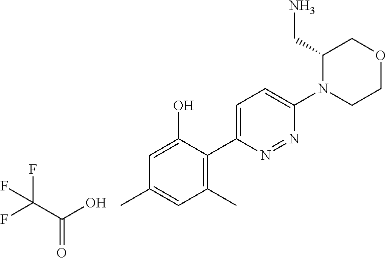

### Int 123.1: tert-butyl N-[[(3R)-4-(6-chloropyridazin-3-yl) morpholin-3-yl]methyl]carbamate

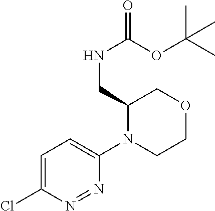 [SMILES: CC(C)(C)OC(=O)NC[C@H]1COCCN1c1ccc(Cl)nn1]

In a microwave vial, under Ar, to a solution of 3,6-dichloropyridazine (2.57 mmol, 404 mg) in 3 ml of NMP was added (R)-tert-butyl (morpholin-3-ylmethyl) carbamate (2.57 mmol, 586 mg) and DIPEA (5.15 mmol, 0.907 mL). The vial was sealed, and the mixture was heated at 180° C. under microwave irradiation for 2.5 h. After cooling to rt, DCM and brine were added. The aqueous layer was extracted twice with DCM. The combined organic layers were dried over MgSO4, filtered and concentrated under reduced pressure. The residue was purified by flash chromatography on silica gel using DCM/MeOH/ACN (from 100/0/0 to 96/2/2) to give an orange oil (356 mg). This oil was purified by flash chromatography on silica gel using DCM/AcOEt (from 10/0 to 6/4) to give the title compound as a white solid (120 mg, 14% yield).

LCMS (Method L): Rt=0.79 min; [M+H]+=329;

### Int 123.2: tert-butyl N-[[(3R)-4-[6-(2-hydroxy-4,6-dimethyl-phenyl) pyridazin-3 yl]morpholin-3-yl]methyl] carbamate

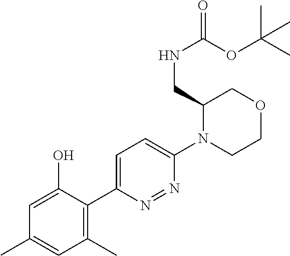 [SMILES: Cc1cc(C)c(-c2ccc(N3CCOC[C@@H]3CNC(=O)OC(C)(C)C)nn2)c(O)c1]

In a microwave vial, under Ar, to a solution of (2-hydroxy-4,6-dimethyl-phenyl) boronic acid (0.197 mmol, 32.8 mg) and tert-butyl N-[[(3R)-4-(6-chloropyridazin-3-yl) morpholin-3-yl]methyl] carbamate (0.152 mmol, 50 mg) in 0.94 ml of dioxane were added sodium carbonate (0.456 mmol, 37 mg) and 0.23 ml of water. The solution was purged with Ar, then Pd(PPh₃)₄ (7.6 μmol, 8.8 mg) was added. The vial was sealed, and the mixture was heated at 100° C. under microwave irradiation for 1 h. After cooling to rt, the reaction mixture was filtered. The filtrate was diluted with EtOAc and washed with water (3 times), and brine. The organic layer was concentrated under reduced pressure. The residue was purified by flash chromatography on silica gel using DCM/MeOH/ACN (from 100/0/0 to 96/2/2) to give the title compound as a white solid (59 mg, 93% yield).

LCMS (Method L): Rt=0.69 min; MS m/z [M+H]+=415

### 2-[6-[(3R)-3-(aminomethyl) morpholin-4-yl] pyridazin-3-yl]-3,5-dimethyl-phenol; 2,2,2-trifluoroacetic acid (Example 123)

To a solution of tert-butyl N-[[(3R)-4-[6-(2-hydroxy-4,6-dimethyl-phenyl) pyridazin-3 yl]morpholin-3-yl]methyl] carbamate (0.14 mmol, 59 mg) in 1 ml of dichloromethane was added dropwise TFA (1.42 mmol, 0.111 mL). The resulting solution was stirred at room temperature overnight. The mixture was concentrated under reduced pressure, diluted in methanol and concentrated under reduced pressure. The residue was suspended in diethyl ether and stirred for 10 min at room temperature. The suspension was filtered off and dried to give the title compound as a light-yellow solid (41.9 mg, 69% yield).

Compounds Example 124 was synthesized analogously to Example 123.

### Example 129: N-[[(3R)-4-[6-(2-hydroxy-4,6-dimethyl-phenyl)pyridazin-3-yl]morpholin-3-yl]methyl]acetamide

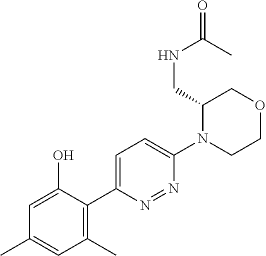 [SMILES: CC(=O)NC[C@@H]1COCCN1c1ccc(-c2c(C)cc(C)cc2O)nn1]

### Int 129.2: [(3R)-4-(6-chloropyridazin-3-yl) morpholin-3-yl]methanamine; hydrochloride

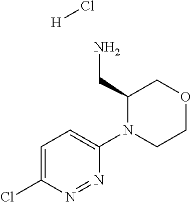

To a solution of tert-butyl N-[[(3R)-4-(6-chloropyridazin-3-yl)morpholin-3-yl]methyl]carbamate (Int 123-1) (0.26 mmol, 88 mg) in 2 ml of DCM was added dropwise HCl 4M/dioxane (2.67 mmol, 0.67 ml). The resulting solution was stirred at room temperature 2 h. The mixture was concentrated under reduced pressure to give [(3R)-4-(6-chloropyridazin-3-yl) morpholin-3-yl]methanamine; hydrochloride as a light-yellow solid (80 mg, 100% yield).

LCMS (Method L): Rt=0.19 min; MS m/z [M+H]⁺=228

### Int 129.3: N-[[(3R)-4-(6-chloropyridazin-3-yl) morpholin-3-yl]methyl]acetamide

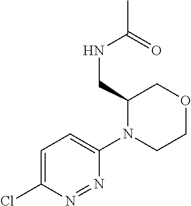 [SMILES: CC(=O)NC[C@H]1COCCN1c1ccc(Cl)nn1]

To a solution of [(3R)-4-(6-chloropyridazin-3-yl) morpholin-3-yl]methanamine; hydrochloride (0.301 mmol, 80 mg) in 2 ml of DCM was added dropwise triethylamine (0.745 mmol, 0.105 mL), then acetic anhydride (0.452 mmol, 42.5 μL) was added. The resulting solution was stirred at room temperature 2 h 30. The mixture was diluted with DCM, washed with a with saturated aqueous solution of NaHCO₃ (3 times). The organic layer was dried over MgSO₄, filtered and concentrated under reduced pressure to give the title compound as a white solid (52 mg, 63% yield).

LCMS (Method L): Rt=0.47 min; MS m/z [M+H]+=271

### N-[[(3R)-4-[6-(2-hydroxy-4,6-dimethyl-phenyl)pyridazin-3-yl]morpholin-3-yl] methyl]acetamide (Example 129)

In a microwave vial, under Ar, to a solution of (2-hydroxy-4,6-dimethyl-phenyl) boronic acid (0.255 mmol, 42.3 mg) and N-[[(3R)-4-(6-chloropyridazin-3-yl)morpholin-3-yl]methyl]acetamide (0.196 mmol, 52 mg) in 1.2 ml of dioxane were added sodium carbonate (0.588 mmol, 48 mg) and 0.30 ml of water. The solution was purged with Ar, then Pd(PPh₃)₄ (9.8 μmol, 11.4 mg) was added. The vial was sealed, and the mixture was heated at 110° C. under microwave irradiation for 1 h. After cooling to rt, the reaction mixture was filtered. The filtrate was diluted with EtOAc and washed with water (3×), and brine. The organic layer was dried over MgSO₄, filtered, and concentrated under reduced pressure. The residue was purified by flash chromatography on silica gel using DCM/MeOH/ACN (from 100/0/0 to 96/2/2), then suspended in pentane and stirred 10 min at room temperature. The suspension was filtered off and dried to give the title compound as a white solid (21 mg, 30% yield).

Compound Example 154 was synthesized analogously to Example 129.

### Example 173: 2-[6-[(2R,6S)-2,6-bis(hydroxymethyl)morpholin-4-yl]pyridazin-3-yl]-3,5-dimethyl-phenol

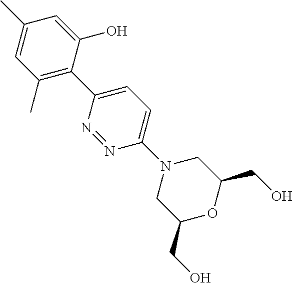 [SMILES: Cc1cc(C)c(-c2ccc(N3C[C@@H](CO)O[C@@H](CO)C3)nn2)c(O)c1]

### Int 173.1: [(2S,6R)-4-(6-chloropyridazin-3-yl)-6-(hydroxymethyl) morpholin-2-yl]methanol

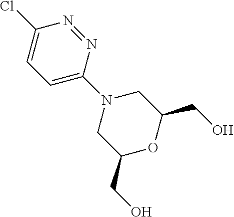 [SMILES: OC[C@@H]1CN(c2ccc(Cl)nn2)C[C@H](CO)O1]

In a microwave vial, under Ar, to a solution of 3,6-dichloropyridazine (1.30 mmol, 200 mg) in 3 ml of 1-butanol was added [(2S,6R)-6-(hydroxymethyl) morpholin-2-yl] methanol hydrochloride (1.30 mmol, 251 mg) and DIPEA (2.60 mmol, 0.458 mL). The vial was sealed, and the mixture was heated at 130° C. under microwave irradiation for 1 h. After cooling to rt, the mixture was concentrated under reduced pressure and DCM was added. The heterogenous mixture was stirred for 10 min, filtered off and dried under vacuum at 40° C. to give the title compound as an orange-yellow solid (125 mg, 71% yield).

LCMS (Method L): Rt=0.43 min; MS m/z [M+H]+=260

### 2-[6-[(2R,6S)-2,6-bis(hydroxymethyl)morpholin-4-yl]pyridazin-3-yl]-3,5-dimethyl-phenol (Example 173)

In a microwave vial, under Ar, to a solution of (2-hydroxy-4,6-dimethyl-phenyl) boronic acid (0.175 mmol, 29 mg) and [(2S,6R)-4-(6-chloropyridazin-3-yl)-6-(hydroxymethyl) morpholin-2-yl] methanol (0.134 mmol, 35 mg) in 0.84 ml of dioxane were added sodium carbonate (0.404 mmol, 33 mg) and 0.21 ml of water. The solution was purged with Ar, then Pd(PPh₃)₄ (6.7 μmol, 7.8 mg) was added. The vial was sealed, and the mixture was heated at 110° C. under microwave irradiation for 1 h. After cooling to rt, the reaction mixture was filtered, and the filtrate was concentrated under reduced pressure. MeOH was added, the mixture was filtered, and the filtrate was concentrated under reduced pressure and the residue was purified by reverse phase chromatography, Column Waters XSelect CSH, C18 5μ 50×250 mm, eluent: acetonitrile/water (+0.1% HCOOH) from 2/98 to 100/0, Flow rate 120 ml/min to give the titled compound as an orange solid (13 mg, 28% yield).

Compounds Examples 178, 198, 199, 200, 201, 258 and 259 were synthesized analogously to Example 173.

### Example 118: 2-[6-[(2R)-2-(hydroxymethyl)azetidin-1-yl]pyridazin-3-yl]-3,5-dimethyl-phenol

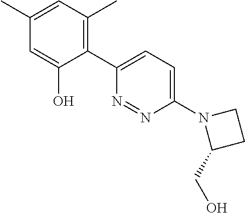 [SMILES: Cc1cc(C)c(-c2ccc(N3CC[C@@H]3CO)nn2)c(O)c1]

### Int 118.1: [(2R)-1-(6-chloropyridazin-3-yl)azetidin-2-yl]methanol

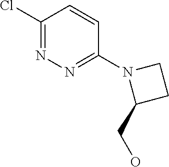 [SMILES: OC[C@@H]1CCN1c1ccc(Cl)nn1]

In a microwave vial, under Ar, to a solution of 3,6-dichloropyridazine (1.68 mmol, 250 mg) and (R)-2-azetidinmethanol (1.8459 mmol, 166 mg) in 2 ml of n-butanol was added N,N-diisopropylamine (3.69 mmol, 477 mg). The vial was sealed, and the mixture was heated at 130° C. under microwave irradiation for 2 h. After cooling to rt, the solvent was concentrated under reduced pressure. DCM was added, the organic layer was washed with water and concentrated under reduced pressure. The residue was purified by flash chromatography on silica gel using AcOEt/MeOH 9/1 to give the title compound as a colorless oil (200 mg, 60% yield).

LCMS (Method U): Rt=0.66 min; MS m/z [M+H]⁺=200;

### 2-[6-[(2R)-2-(hydroxymethyl)azetidin-1-yl]pyridazin-3-yl]-3,5-dimethyl-phenol (Example 118)

In a microwave vial, under Ar, to a solution of (2-hydroxy-4,6-dimethylphenyl)boronic acid (1.04 mmol, 174 mg) and [(2R)-1-(6-chloropyridazin-3-yl)azetidin-2-yl]methanol (0.95 mmol, 190 mg) in 6 ml of 1,4-dioxane were added sodium carbonate (2.85 mmol, 302 mg) and 1.5 ml of water. The solution was purged with Ar, then Pd(PPh₃)₄ (0.06 mmol, 66 mg) was added. The vial was sealed, and the mixture was heated at 110° C. under microwave irradiation for 2 h. After cooling to rt, the solvent was concentrated under reduced pressure. EtOAc was added and the organic layer was washed with water (3 times), dried over Na₂SO₄, filtered and concentrated under reduced pressure. The residue was purified by flash chromatography on silica gel using DCM/MeOH/ACN (97/1.5/1.5) to give the title compound as a white solid (85 mg, 31% yield).

Compound Example 134 was synthesized analogously to Example 118 using Int 8.1 ([(2S)-4-(6-chloropyridazin-3-yl)morpholin-2-yl]methanol).

### Example 273: 3-(hydroxymethyl)-1-[6-[2-hydroxy-6-methyl-4-(trifluoromethyl) phenyl]pyridazin-3-yl]piperidin-2-one

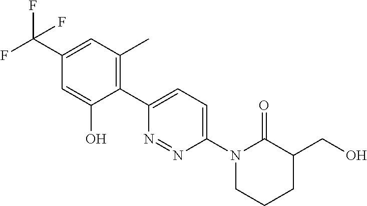 [SMILES: Cc1cc(C(F)(F)F)cc(O)c1-c1ccc(N2CCCC(CO)C2=O)nn1]

### Int 273.1: 3-[[tert-butyl(dimethyl)silyl]oxymethyl]piperidin-2-one

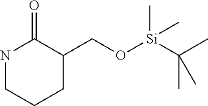 [SMILES: CC(C)(C)[Si](C)(C)OCC1CCCNC1=O]

Under Ar, to a solution of 3-(hydroxymethyl)piperidin-2-one (4.5 mmol, 600 mg) in 3.6 ml of DMF are added 4-dimethylaminopyridine (1.35 mmol, 165 mg) and triethylamine (5.4 mmol, 550 mg). tert-Butyldimethylchlorosilane (4.96 mmol, 762 mg) was slowly added. The reaction mixture was stirred at room temperature for 16 h. The reaction mixture was diluted with AcOEt and poured into 5% aqueous NaHCO₃ solution. The organic layer was washed with 5% aqueous NaHCO₃ solution (twice) and with brine, dried over Na₂SO₄, filtered and concentrated under reduced pressure. The residue was purified by flash chromatography on silica gel using AcOEt/MeOH (100/0 to 90/10) to give the title compound as a colorless oil (840 mg, 77% yield).

LCMS (Method L): Rt=1.09 min; MS m/z [M+H]⁺=244.1;

### 3-(hydroxymethyl)-1-[6-[2-hydroxy-6-methyl-4-(trifluoromethyl)phenyl]pyridazin-3-yl]piperidin-2-one (Example 273)

Under Ar, a solution of 3-[[tert-butyl(dimethyl)silyl]oxymethyl]-1-[6-[2-hydroxy-6-methyl-4-(trifluoromethyl)phenyl]pyridazin-3-yl]piperidin-2-one (obtained similarly to Example 130 from 3-[[tert-butyl(dimethyl)silyl]oxymethyl]piperidin-2-one, 1.39 mmol, 900 mg) in 3 ml of THF was cooled at +5° C., then a solution of 6 ml of acetic acid and 1 ml of water was added dropwise. The reaction mixture was stirred at room temperature overnight then heated at 50° C. for 48 h. After cooling to rt, the reaction mixture was poured into DCM and water. The organic layer was washed with NaHCO₃ saturated solution, dried over Na₂SO₄, filtered and concentrated under reduced pressure. The residue was purified by flash chromatography on silica gel using Heptane/(AcOEt 3/1 EtOH) 8/2 to give the title compound as a white solid (420 mg, 79% yield).

Compound Examples 286, 316 and 368 were synthesized analogously to Example 273. Compound Examples 419 and 420 were separated enantiomerically from Compound Example 368, analogously to Compound Examples 287 and 288, using a column ChiralPak® IC.

### Example 116: 2-[6-[(2S)-2-(hydroxymethyl)morpholin-4-yl]-4-methyl-pyridazin-3-yl]-5-methoxy-phenol

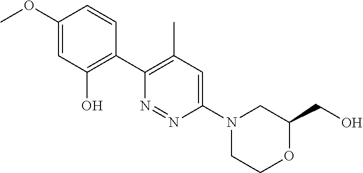 [SMILES: COc1ccc(-c2nnc(N3CCO[C@H](CO)C3)cc2C)c(O)c1]

Under Ar, to a solution of 5-methoxy-2-(4,4,5,5-tetramethyl-1,3,2-dioxaborolan-2-yl)phenol (1.2 mmol, 300 mg) and [(2S)-4-(6-chloro-5-methyl-pyridazin-3-yl)morpholin-2-yl]methanol (Int 85.1-0.8 mmol, 195 mg) in 5 ml of 1,4-dioxane was added a 2N sodium carbonate aqueous solution (2.16 mmol, 1.08 ml) then tetrakis(triphenylphosphine)palladium(0) (0.04 mmol, 46 mg). The mixture was stirred at 100° C. for 3 h. After cooling down, the resulting mixture was diluted with EtOAc and the aqueous layer extracted again with a 70/30 mixture of dichloromethane/isopropanol. The combined organic layers were washed successively with water and brine. The organic layer was dried over Na₂SO₄, filtered and concentrated under reduced pressure. The residue was triturated in methanol, filtered off and the solid washed with diethylether to give the title compound (170 mg, 64% yield) as a yellow solid.

Compounds Examples 149 and 177 were synthesized analogously to Example 116.

### Example 128: 2-[6-[(3S)-3-(dimethylamino)-1-piperidyl]pyridazin-3-yl]-3,5-dimethyl-phenol

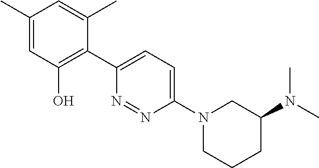

### Int 128.1: (3S)-1-(6-chloropyridazin-3-yl)-N,N-dimethyl-piperidin-3-amine

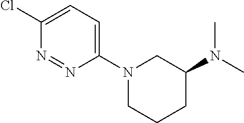 [SMILES: CN(C)[C@H]1CCCN(c2ccc(Cl)nn2)C1]

A solution of 3,6-dichloropyridazine (1.42 mmol, 211 mg), (3S)—N,N-dimethylpiperidin-3-amine dihydrochloride (1.7 mmol, 342 mg) and triethylamine (4.53 mmol, 632 μl) in 14 ml of n-butanol was stirred at 120° C. for 18 h. The mixture was cooled down, concentrated under reduced pressure and the residue was diluted with 50 ml of EtOAc then washed successively with 20 ml of a saturated aqueous sodium bicarbonate solution and 10 ml of brine. The organic layer was dried over Na₂SO₄, filtered, and concentrated under reduced pressure. The residue was purified by flash chromatography on silica gel eluting with a gradient of 0 to 10% of methanol in dichloromethane to give the title compound (165 mg, 48% yield) as a beige solid.

LCMS (Method L): Rt=0.46 min; MS m/z [M+H]⁺ 241;

### 2-[6-[(3S)-3-(dimethylamino)-1-piperidyl]pyridazin-3-yl]-3,5-dimethyl-phenol (Example 128)

Under Ar, to a solution of (2-hydroxy-4,6-dimethyl-phenyl)boronic acid (1.02 mmol, 169 mg) and (3S)-1-(6-chloropyridazin-3-yl)-N,N-dimethyl-piperidin-3-amine (0.68 mmol, 163 mg) in 4 ml of 1,4-dioxane was added a 2N sodium carbonate aqueous solution (1.83 mmol, 914 μl) then tetrakis(triphenylphosphine)palladium(0) (0.034 mmol, 39 mg). The mixture was stirred at 100° C. for 3 h. After cooling down, the resulting mixture was diluted with 20 ml of EtOAc and the organic layer was washed successively with water and brine. The organic layer was dried over Na₂SO₄, filtered and concentrated under reduced pressure. The residue was purified by flash chromatography on silica gel eluting with a gradient of 0 to 15% of MeOH in DCM to give a yellow solid residue which was triturated in a mixture of dichloromethane and pentane to give the title compound (120 mg, 54% yield) as a as a beige solid.

### Example 119: 2-[6-[(2S)-2-(hydroxymethyl)morpholin-4-yl]-4-methyl-pyridazin-3-yl]-5-methoxy-3-methyl-phenol

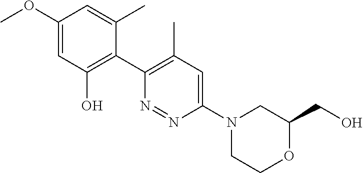 [SMILES: COc1cc(C)c(-c2nnc(N3CCO[C@H](CO)C3)cc2C)c(O)c1]

Following procedure of Example 85, [(2S)-4-(6-chloro-5-methyl-pyridazin-3-yl)morpholin-2-yl]methanol (Int 85.1-1.026 mmol, 250 mg) and 5-methoxy-3-methyl-2-(4,4,5,5-tetramethyl-1,3,2-dioxaborolan-2-yl)phenol (Int 96.2-1.54 mmol, 406 mg) give the title compound as an orange oil (30 mg, 8% yield)

### Example 133: 5-chloro-2-[6-[(2S)-2-(hydroxymethyl)morpholin-4-yl]-5-methyl-pyridazin-3-yl]-3-methyl-phenol

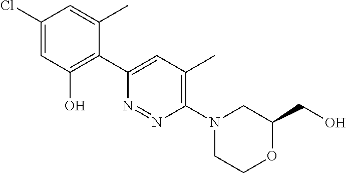 [SMILES: Cc1cc(-c2c(C)cc(Cl)cc2O)nnc1N1CCO[C@H](CO)C1]

### Int 133.1: [(2S)-4-(6-chloro-4-methyl-pyridazin-3-yl)morpholin-2-yl]methanol

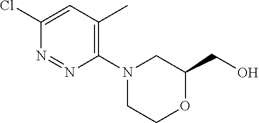 [SMILES: Cc1cc(Cl)nnc1N1CCO[C@H](CO)C1]

A solution of 3,6-dichloro-4-methyl-pyridazine (24.5 mmol, 4 g), [(2S)-morpholin-2-yl]methanol; hydrochloride (24.5 mmol, 3.77 g) and triethylamine (54 mmol, 7.52 ml) in 50 ml of n-butanol was stirred at 130° C. for 11 h. The mixture was cooled down, diluted with EtOAc and diethylether then filtered. The filtrate was concentrated under reduced pressure and the residue was purified by flash chromatography on silica gel eluting with a gradient of 0 to 100% of a 99/1 mixture EtOAc/MeOH in cyclohexane to give the title compound (376 mg, 6% yield) as a white solid.

LCMS (Method L): Rt=0.51 min; MS m/z [M+H]⁺ 244;

### 5-chloro-2-[6-[(2S)-2-(hydroxymethyl)morpholin-4-yl]-5-methyl-pyridazin-3-yl]-3-methyl-phenol (Example 133)

Under Ar, to a solution of (4-chloro-2-hydroxy-6-methyl-phenyl)boronic acid (0.74 mmol, 138 mg) and [(2S)-4-(6-chloro-4-methyl-pyridazin-3-yl)morpholin-2-yl]methanol (Int 133.1-0.49 mmol, 120 mg) in 4 ml of 1,4-dioxane was added a 2N sodium carbonate aqueous solution (1.33 mmol, 665 μl) then tetrakis(triphenylphosphine)palladium(0) (0.025 mmol, 28 mg). The mixture was stirred at 100° C. for 3 h. After cooling down, the resulting mixture was diluted with EtOAc and diethylether. The organic layer was washed successively with a saturated aqueous sodium bicarbonate solution and brine. The organic layer was dried over Na₂SO₄, filtered and concentrated under reduced pressure. The residue was purified by flash chromatography on silica gel using a gradient of DCM/MeOH/ACN (from 98/1/1 to 94/3/3) to give the title compound as a yellow solid (29 mg, 17% yield).

### Examples 135 and 136: 2-[6-[(2S)-2-(1-aminocyclopropyl)morpholin-4-yl]pyridazin-3-yl]-3,5-dimethyl-phenol and 2-[6-[(2R)-2-(1-aminocyclopropyl)morpholin-4-yl]pyridazin-3-yl]-3,5-dimethyl-phenol

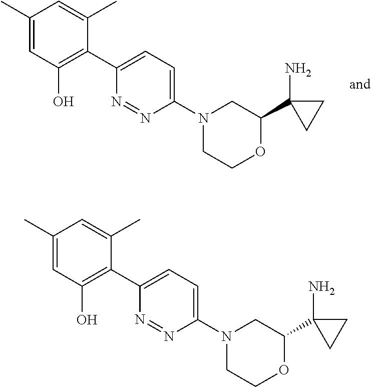 [SMILES: Cc1cc(C)c(-c2ccc(N3CCO[C@H](C4(N)CC4)C3)nn2)c(O)c1 ; Cc1cc(C)c(-c2ccc(N3CCO[C@@H](C4(N)CC4)C3)nn2)c(O)c1]

### Int 135.1: 1-morpholin-2-ylcyclopropanamine; 2,2,2-trifluoroacetic acid

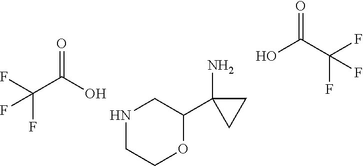

To a solution of tert-butyl 2-(1-aminocyclopropyl)morpholine-4-carboxylate (2.48 mmol, 600 mg) in 9 ml of dichloromethane was added 1 ml of trifluoroacetic acid and the mixture was stirred for 5 h at rt then concentrated under reduced pressure to give the title compound (300 mg, 85% yield) as a yellow oil.

LCMS (Method H) Rt=0.24 min; MS m/z [M+H]⁺ 143.

### Int 135.2: 1-[4-(6-chloropyridazin-3-yl)morpholin-2-yl]cyclopropanamine

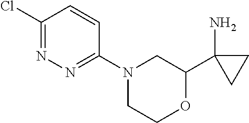 [SMILES: NC1(C2CN(c3ccc(Cl)nn3)CCO2)CC1]

Following procedure of Int 128.1, 1-morpholin-2-ylcyclopropanamine; 2,2,2-trifluoroacetic acid (3.69 mmol, 525 mg) and 3,6-dichloropyridazine (3.36 mmol, 500 mg) give the title compound as a yellow oil (850 mg, 99% yield).

LCMS (Method H) Rt=0.80 min; MS m/z [M+H]⁺ 255

### Int 135.3: 2-[6-[2-(1-aminocyclopropyl)morpholin-4-yl]pyridazin-3-yl]-3,5-dimethyl-phenol (racemate)

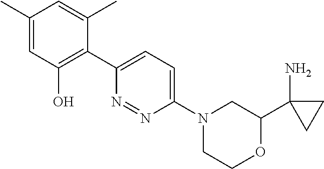 [SMILES: Cc1cc(C)c(-c2ccc(N3CCOC(C4(N)CC4)C3)nn2)c(O)c1]

Following procedure of Example 85, 1-[4-(6-chloropyridazin-3-yl)morpholin-2-yl]cyclopropanamine (1.77 mmol, 450 mg) give the title compound as an orange oil (70 mg, 11% yield).

LCMS (Method L): Rt=0.57 min; MS m/z [M+H]⁺ 341

### 2-[6-[(2S)-2-(1-aminocyclopropyl)morpholin-4-yl]pyridazin-3-yl]-3,5-dimethyl-phenol and 2-[6-[(2R)-2-(1-aminocyclopropyl)morpholin-4-yl]pyridazin-3-yl]-3,5-dimethyl-phenol (Example 135: Enantiomer 1 and Example 136: Enantiomer 2)

The 2 enantiomers of compound 2-[6-[2-(1-aminocyclopropyl)morpholin-4-yl]pyridazin-3-yl]-3,5-dimethyl-phenol were separated by chiral normal phase chromatography (Column: Daicel Chiralpcel® OD, 350×30 mm I.D., 5 μm particle size; Mobile phase: Eluent B: EtOH (0.1% TEA), Isocratic elution: 80% Phase B; Flow rate: 45 ml/min) to give both enantiomers, first one eluting at Rt=7.9 min (13.5 mg, Example 135, Enantiomer 1) as a white solid, and second eluting at Rt=15.3 min (14.2 mg, Example 136, Enantiomer 2) as a white solid. (The absolute configurations cannot be assigned).

### Example 144: 2-[4-(difluoromethyl)-6-[(2S)-2-(hydroxymethyl)morpholin-4-yl]pyridazin-3-yl]-5-methyl-phenol

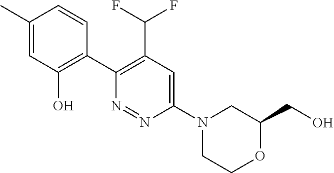 [SMILES: Cc1ccc(-c2nnc(N3CCO[C@H](CO)C3)cc2C(F)F)c(O)c1]

### Int 144.1: 3,6-dichloro-4-(difluoromethyl)pyridazine

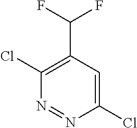 [SMILES: FC(F)c1cc(Cl)nnc1Cl]

To a suspension of 3,6-dichloropyridazine (13.42 mmol, 2 g) in 20 ml of water was added 2,2-difluoroacetic acid (18.8 mmol, 1.18 ml) and the mixture was stirred at 55° C. Then was added silver nitrate (2.68 mmol, 456 mg) solubilized in 2 ml of water and trifluoroacetic acid (2.68 mmol, 200 μl). To the mixture stirred at 55° C. was added dropwise a solution of ammonium persulfate (21.5 mmol, 4.9 g) in 9 ml of water and the mixture was stirred for 2 h at 75° C. then cooled to rt and quenched with 50 ml of a saturated aqueous sodium bicarbonate solution and 100 ml of dichloromethane under stirring. The organic layer was dried over MgSO₄, filtered, and concentrated under reduced pressure and the residue was purified by flash chromatography on silica gel eluting with a gradient of 0 to 100% of diisopropylether in petroleum ether to give the title compound (820 mg, 31% yield) as a colorless liquid.

LCMS (Method L): Rt=0.96 min; MS m/z [M+H]⁺ 199.

### Int 144.2 and Int 170.1: [(2S)-4-[6-chloro-5-(difluoromethyl)pyridazin-3-yl]morpholin-2-yl]methanol (Int 144.2) and [(2S)-4-[6-chloro-4-(difluoromethyl)pyridazin-3-yl]morpholin-2-yl]methanol (Int 170.1)

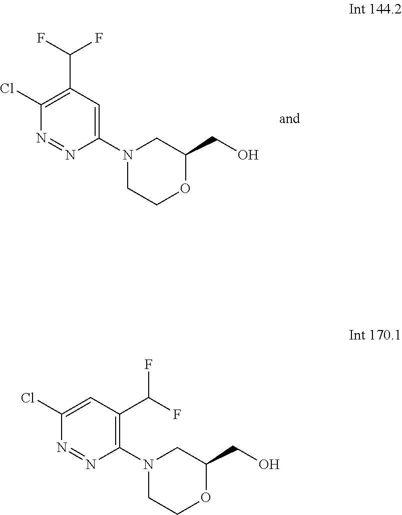 [SMILES: OC[C@@H]1CN(c2cc(C(F)F)c(Cl)nn2)CCO1 ; OC[C@@H]1CN(c2nnc(Cl)cc2C(F)F)CCO1]

A solution of 3,6-dichloro-4-(difluoromethyl)pyridazine (7.94 mmol, 1.58 g), [(2S)-morpholin-2-yl]methanol; hydrochloride (7.94 mmol, 1.22 g) and triethylamine (17.5 mmol, 1.43 ml) in 20 ml of n-butanol was stirred at 130° C. for 6 h. The mixture was cooled down, concentrated under reduced pressure and the residue was taken with 50 ml of EtOAc and filtered off. The solid was rinsed with EtOAc and the filtrate concentrated under reduced pressure. The residue was purified by flash chromatography on silica gel eluting with a gradient of 0 to 100% of a 99/1 mixture EtOAc/MeOH in cyclohexane to give the title compounds Int 144.2 (1.44 g, 65% yield) and Int 170.1 (555 mg, 25% yield) as a yellow solid.

### LCMS (Method L):

Int 144.2: Rt=0.84 min; MS m/z [M+H]⁺ 280

Int 170.1: Rt=0.87 min; MS m/z [M+H]⁺ 280

### 2-[4-(difluoromethyl)-6-[(2S)-2-(hydroxymethyl)morpholin-4-yl]pyridazin-3-yl]-5-methyl-phenol (Example 144)

Following procedure of Example 133, [(2S)-4-[6-chloro-5-(difluoromethyl)pyridazin-3-yl]morpholin-2-yl]methanol (Int 144.2-0.715 mmol, 200 mg) gave the title compound as white solid (136 mg, 54% yield)

Compound Example 145 was synthesized analogously to Example 144.

### Example 170: 2-[5-(difluoromethyl)-6-[(2S)-2-(hydroxymethyl)morpholin-4-yl]pyridazin-3-yl]-3,5-dimethyl-phenol

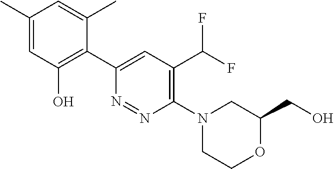 [SMILES: Cc1cc(C)c(-c2cc(C(F)F)c(N3CCO[C@H](CO)C3)nn2)c(O)c1]

Under Ar, to a solution of (2-hydroxy-4,6-dimethyl-phenyl)boronic acid (1.43 mmol, 237 mg) and [(2S)-4-[6-chloro-4-(difluoromethyl)pyridazin-3-yl]morpholin-2-yl]methanol (Int 170.1-0.715 mmol, 200 mg) in 10 ml of 1,4-dioxane was added a 2N sodium carbonate aqueous solution (1.93 mmol, 0.965 ml) then Tetrakis(triphenylphosphine)palladium(0) (0.036 mmol, 41 mg). The mixture was stirred at 100° C. for 3 h. After cooling down, EtOAc and diethylether were added and the resulting mixture was washed successively with aqueous saturated NaHCO₃ and water. The organic layer was dried over Na₂SO₄, filtered, and concentrated under reduced pressure. The residue was purified by flash chromatography on silica gel eluting with a gradient of DCM/MeOH/ACN (from 98/1/1 to 94/3/3) to give a yellow solid residue which was again purified by prep-HPLC (column: Sunfire prep C18 OBD; 250*50 mm*10 μm; mobile phase: [water (TFA 10⁻² N)-ACN]; B %: 10%-100% in 35 min. at 50 ml/min) to give the title compound as a white solid (111 mg, 42% yield).

Compounds Examples 171, 172, 194, 247, 248 and 249 were synthesized analogously to Example 170.

### Example 155: 2-[6-[(2S)-2-(hydroxymethyl)morpholin-4-yl]-4-(trifluoromethyl)pyridazin-3-yl]-5-methyl-phenol

### Int 155.1: [(2S)-4-[6-chloro-5-(trifluoromethyl)pyridazin-3-yl]morpholin-2-yl]methanol

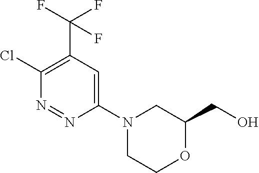 [SMILES: OC[C@@H]1CN(c2cc(C(F)(F)F)c(Cl)nn2)CCO1]

A solution of 3,6-dichloro-4-(trifluoromethyl)pyridazinel (4.61 mmol, 1 g), [(2S)-morpholin-2-yl]methanol; hydrochloride (5.07 mmol, 778 mg) and triethylamine (10.14 mmol, 1.41 ml) in 10 ml of n-butanol was stirred at 120° C. for 4 h. The mixture was cooled down, concentrated under reduced pressure and the residue was diluted with 250 ml of EtOAc then washed with 100 ml of water. The organic layer was dried over Na₂SO₄, filtered and concentrated under reduced pressure. The residue was purified by flash chromatography on silica gel eluting with a gradient of DCM/MeOH/ACN (from 98/1/1 to 94/3/3) to give the title compound (772 mg, 56% yield) as a yellow oil.

LCMS (Method X): Rt=0.68 min; MS m/z [M+H]⁺=298.

### 2-[6-[(2S)-2-(hydroxymethyl)morpholin-4-yl]-4-(trifluoromethyl)pyridazin-3-yl]-5-methyl-phenol (Example 155)

Following procedure of Example 133 [(2S)-4-[6-chloro-5-(trifluoromethyl)pyridazin-3-yl]morpholin-2-yl]methanol (Int 155.1-0.67 mmol, 200 mg) gave the title compound as white solid (186 mg, 74% yield).

Compound Example 160 was synthesized analogously to Example 155.

### Example 158: 6-(2-hydroxy-4,6-dimethyl-phenyl)-3-[(2S)-2-(hydroxymethyl)morpholin-4-yl]pyridazine-4-carbonitrile

### Int 158.1: 6-chloro-3-[(2S)-2-(hydroxymethyl)morpholin-4-yl]pyridazine-4-carbonitrile

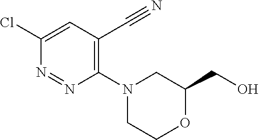 [SMILES: N#Cc1cc(Cl)nnc1N1CCO[C@H](CO)C1]

Following procedure of Int 155.1, 3,6-dichloropyridazine-4-carbonitrile (4.02 mmol, 700 mg) give the title compound as yellow oil (779 mg, 76% yield).

LCMS (Method X): Rt=0.92 min; MS m/z [M+H]⁺ 255.

### 6-(2-hydroxy-4,6-dimethyl-phenyl)-3-[(2S)-2-(hydroxymethyl)morpholin-4-yl]pyridazine-4-carbonitrile (Example 158)

Following procedure of Example 133, 6-chloro-3-[(2S)-2-(hydroxymethyl)morpholin-4-yl]pyridazine-4-carbonitrile (Int 158.1-0.785 mmol, 200 mg) gave the title compound as white solid (154 mg, 58% yield).

Compounds Examples 159 and 163 was synthesized analogously to Example 158.

### Examples 175 and 176: 6-[6-(2-hydroxy-4,6-dimethyl-phenyl)pyridazin-3-yl]-6-azaspiro[3.5]nonan-2-ol Isomer 1 and 6-[6-(2-hydroxy-4,6-dimethyl-phenyl)pyridazin-3-yl]-6-azaspiro[3.5]nonan-2-ol Isomer 2

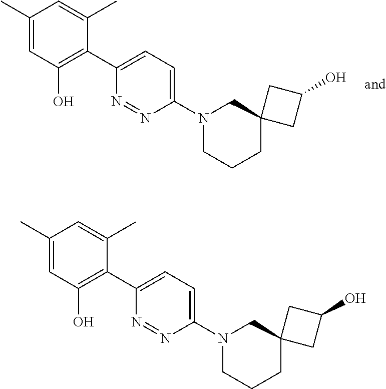 [SMILES: Cc1cc(C)c(-c2ccc(N3CCC[C@]4(C3)C[C@H](O)C4)nn2)c(O)c1 ; Cc1cc(C)c(-c2ccc(N3CCC[C@]4(C3)C[C@@H](O)C4)nn2)c(O)c1]

### Int 175.1: 6-(6-chloropyridazin-3-yl)-6-azaspiro[3.5]nonan-2-ol

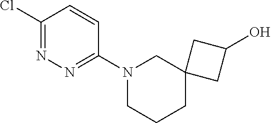 [SMILES: OC1CC2(CCCN(c3ccc(Cl)nn3)C2)C1]

A solution of 3,6-dichloropyridazine (3.02 mmol, 450 mg), 6-azaspiro[3.5]nonan-2-ol; hydrochloride (3.32 mmol, 590 mg) and triethylamine (6.64 mmol, 0.93 ml) in 5 ml of n-butanol was stirred at 130° C. for 3 h. The mixture was cooled down, concentrated under reduced pressure and the residue was diluted with 150 ml of EtOAc then washed with 150 ml of water. The organic layer was dried over Na₂SO₄, filtered, and concentrated under reduced pressure. The residue was purified by flash chromatography on silica gel eluting with a gradient of 0 to 100% of EtOAc in cyclohexane to give the title compound (452 mg, 59% yield) as a beige solid.

LCMS (Method L): Rt=1.23 min; MS m/z [M+H]⁺ 254.

### 6-[6-(2-hydroxy-4,6-dimethyl-phenyl)pyridazin-3-yl]-6-azaspiro[3.5]nonan-2-ol Isomer 1 and 6-[6-(2-hydroxy-4,6-dimethyl-phenyl)pyridazin-3-yl]-6-azaspiro[3.5]nonan-2-ol Isomer 2 (Example 175: isomer 1 and Example 176: isomer 2)

Under Ar, to a solution of 6-(6-chloropyridazin-3-yl)-6-azaspiro[3.5]nonan-2-ol (1.78 mmol, 452 mg) and (2-hydroxy-4,6-dimethyl-phenyl)boronic acid (2.67 mmol, 444 mg) in 8 ml of 1,4-dioxane was added a 2N sodium carbonate aqueous solution (4.81 mmol, 2.4 ml) then tetrakis(triphenylphosphine)palladium(0) (0.09 mmol, 103 mg). The mixture was stirred at 100° C. for 3 h. After cooling down, EtOAc and diethylether were added and the resulting mixture was washed successively with aqueous saturated NaHCO₃ and water. The organic layer was dried over Na₂SO₄, filtered, and concentrated under reduced pressure. The residue was purified by flash chromatography on silica gel eluting with a gradient of 0 to 100% of EtOAc in cyclohexane to give a mixture of 6-[6-(2-hydroxy-4,6-dimethyl-phenyl)pyridazin-3-yl]-6-azaspiro[3.5]nonan-2-ol Isomer. The residue was purified by flash chromatography on silica gel eluting with a gradient of 0 to 100% of EtOAc in cyclohexane to give a mixture of 6-[6-(2-hydroxy-4,6-dimethyl-phenyl)pyridazin-3-yl]-6-azaspiro[3.5]nonan-2-ol 2 and 6-[6-(2-hydroxy-4,6-dimethyl-phenyl)pyridazin-3-yl]-6-azaspiro[3.5]nonan-2-ol Isomer 1. Isomer 2 and 6-[6-(2-hydroxy-4,6-dimethyl-phenyl)pyridazin-3-yl]-6-azaspiro[3.5]nonan-2-ol Isomer 1.

LCMS (Method L): Rt=0.70 min and 0.74 min; MS m/z [M+H]⁺=340.

The isomeric mixture Isomer 1/Isomer 2 was separated by semi-preparative chiral normal phase chromatography (Column: Daicel Chiralpak® IC, 350×76.5 mm, 20 μm particle size; Mobile phase: Eluent B: EtOH (0.1% TEA), Isocratic elution: 80% Phase B; Flow rate: 400 ml/min) to provide the 2 stereoisomers, first one eluting at Rt=16.6 min (175 mg, Example 175, isomer 1) as a white solid, and second one eluting at Rt=17.8 min (219 mg, Example 176, isomer 2) as a white solid.

### Examples 186 and 187: 2-[4-(hydroxymethyl)-6-[(2S)-2-(hydroxymethyl)morpholin-4-yl]pyridazin-3-yl]-3,5-dimethyl-phenol and 2-[5-(hydroxymethyl)-6-[(2S)-2-(hydroxymethyl)morpholin-4-yl]pyridazin-3-yl]-3,5-dimethyl-phenol

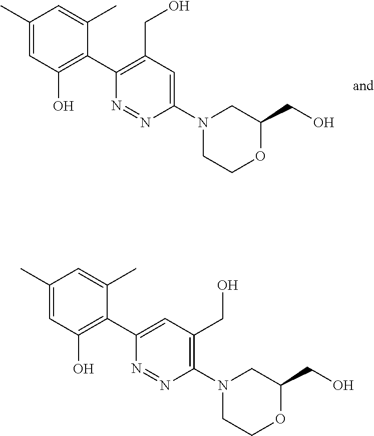 [SMILES: Cc1cc(C)c(-c2nnc(N3CCO[C@H](CO)C3)cc2CO)c(O)c1 ; Cc1cc(C)c(-c2cc(CO)c(N3CCO[C@H](CO)C3)nn2)c(O)c1]

### Int 186.1 and Int 187.1: [3-chloro-6-[(2S)-2-(hydroxymethyl)morpholin-4-yl]pyridazin-4-yl]methanol (Int 186.1) and [6-chloro-3-[(2S)-2-(hydroxymethyl)morpholin-4-yl]pyridazin-4-yl]methanol (Int 187.1)

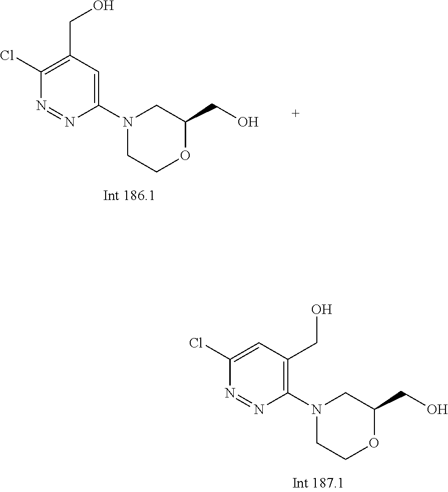 [SMILES: OCc1cc(N2CCO[C@H](CO)C2)nnc1Cl ; OCc1cc(Cl)nnc1N1CCO[C@H](CO)C1]

A solution of (3,6-dichloropyridazin-4-yl)methanol (13 mmol, 2.3 g), (3S)—N,N-dimethylpiperidin-3-amine dihydrochloride (14 mmol, 2.2 g) and triethylamine (28 mmol, 3.9 ml) in 40 ml of n-butanol was stirred at 130° C. for 3 h. The mixture was cooled down, concentrated under reduced pressure and the residue was diluted with 200 ml of EtOAc then washed with 150 ml of water. The organic layer was dried over Na₂SO₄, filtered and concentrated under reduced pressure. The residue was purified by flash chromatography on silica gel eluting with a gradient of 0 to 10% of methanol in dichloromethane to give the title compounds as a mixture (165 mg, 36% yield) and as yellow oil.

LCMS (Method L): Int 186.1 and Int 187.1: Rt=0.83 min; MS m/z [M+H]⁺ 260.

### 2-[4-(hydroxymethyl)-6-[(2S)-2-(hydroxymethyl)morpholin-4-yl]pyridazin-3-yl]-3,5-dimethyl-phenol and 2-[5-(hydroxymethyl)-6-[(2S)-2-(hydroxymethyl)morpholin-4-yl]pyridazin-3-yl]-3,5-dimethyl-phenol (Example 186 and Example 187)

Following procedure of Example 133, a mixture of [3-chloro-6-[(2S)-2-(hydroxymethyl)morpholin-4-yl]pyridazin-4-yl]methanol (Int 186.1) and [6-chloro-3-[(2S)-2-(hydroxymethyl)morpholin-4-yl]pyridazin-4-yl]methanol (Int 187.1) (2.5 mmol, 1.3 g) gives 2-[4-(hydroxymethyl)-6-[(2S)-2-(hydroxymethyl)morpholin-4-yl]pyridazin-3-yl]-3,5-dimethyl-phenol (Example 186-385 mg, 45% yield) and 2-[5-(hydroxymethyl)-6-[(2S)-2-(hydroxymethyl)morpholin-4-yl]pyridazin-3-yl]-3,5-dimethyl-phenol (Example 187-96 mg, 11% yield) as white solids.

Compounds examples 188 and 189 were synthesized analogously to Example 186 and 187.

### Examples 197: 2-[6-[rac-(4aR,8aS)-2,3,4a,5,6,7,8,8a-octahydropyrido[4,3-b][1,4]oxazin-4-yl]pyridazin-3-yl]-3-methyl-5-(trifluoromethyl)phenol

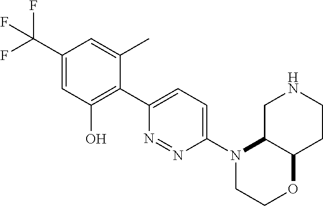

### Int 197.1: benzyl rac-(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazine-6-carboxylate

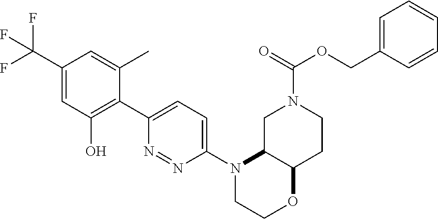

Following procedure of Example 133, benzyl rac-(4aR,8aS)-4-(6-chloropyridazin-3-yl)-3,4a,5,7,8,8a-hexahydro-2H-pyrido[4,3-b][1,4]oxazine-6-carboxylate (Int 10.8-2.57 mmol, 1 g) give the title compound (1.38 g, 82% yield) as a yellow oil.

LCMS (Method V): Rt=1.14 min; MS m/z [M+H]⁺ 529.

### 3-methyl-2-[6-[rac-(4aR,8aS)-2,3,4a,5,6,7,8,8a-octahydropyrido[4,3-b][1,4]oxazin-4-yl]pyridazin-3-yl]-5-(trifluoromethyl)phenol (Example 197)

To a suspension of benzyl rac-(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazine-6-carboxylate (2.61 mmol, 1.38 g) in 40 ml of EtOH was added ammonium formate (7.83 mmol, 494 mg) and 10% Pd/C (494 mg) and the resulting mixture was stirred at 80° C. under microwave irradiation for 1 h. After cooling down to RT, the mixture was filtered off over a pad of Decalite® and the cake rinsed with 50 ml of EtOH. The organic layer was concentrated under reduced pressure and the residue was diluted with 200 ml of dichloromethane and washed twice with 200 ml of an aqueous saturated sodium bicarbonate solution, dried over Na₂SO₄, filtered and concentrated under reduced pressure. The residue was purified by flash chromatography on silica gel using a gradient of DCM/MeOH/ACN (from 100/0/0 to 80/10/10) to give the title compound as a white solid (660 mg, 64% yield).

Compounds Examples 195 and 221 were synthesized analogously to Examples 197.

### Example 251 and 252: 2-[6-[(4aR,8aS)-2,3,4a,5,6,7,8,8a-octahydropyrido[4,3-b][1,4]oxazin-4-yl]pyridazin-3-yl]-3-methyl-5-(trifluoromethyl)phenol and 2-[6-[(4aS,8aR)-2,3,4a,5,6,7,8,8a-octahydropyrido[4,3-b][1,4]oxazin-4-yl]pyridazin-3-yl]-3-methyl-5-(trifluoromethyl)phenol

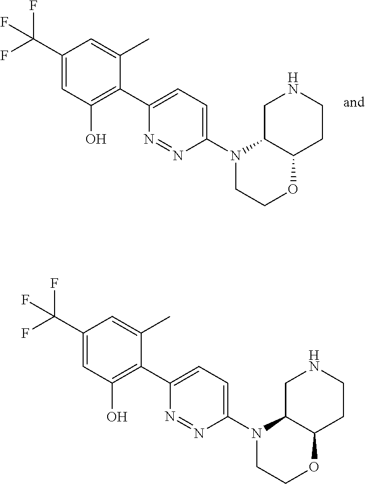 [SMILES: Cc1cc(C(F)(F)F)cc(O)c1-c1ccc(N2CCO[C@H]3CCNC[C@H]32)nn1 ; Cc1cc(C(F)(F)F)cc(O)c1-c1ccc(N2CCO[C@@H]3CCNC[C@@H]32)nn1]

3-methyl-2-[6-[rac-(4aR,8aS)-2,3,4a,5,6,7,8,8a-octahydropyrido[4,3-b][1,4]oxazin-4-yl]pyridazin-3-yl]-5-(trifluoromethyl)phenol (Example 197-174 mg) was separated by Supercritical Fluidic Chromatography (SFC—Column: Daicel Chiralpak® AD-H, 250×30 mm I.D., 5 μm particle size; Mobile phase: CO₂/Ethanol (+0.1% Triethylamine) 40%; flow rate: 200 mL/min; outlet pressure: 100 bar; 8 injections; UV detection: 265 nm) to provide 2-[6-[(4aR,8aS)-2,3,4a,5,6,7,8,8a-octahydropyrido[4,3-b][1,4]oxazin-4-yl]pyridazin-3-yl]-3-methyl-5-(trifluoromethyl)phenol, first eluting enantiomer at Rt=5.9 min (62 mg, Example 251, Enantiomer 1), as a white solid, and 2-[6-[(4aS,8aR)-2,3,4a,5,6,7,8,8a-octahydropyrido[4,3-b][1,4]oxazin-4-yl]pyridazin-3-yl]-3-methyl-5-(trifluoromethyl) phenol, second eluting enantiomer at Rt=13.2 min (70 mg, Example 252, Enantiomer 2), as a white solid. The absolute configurations cannot be assigned at this stage.

Compounds Examples 314, 315 and 308, 309 were obtained by preparative chiral SFC separation analogously to Example 251 and 252.

### Examples 226: 2-[6-[rac-(4aR,8aS)-6-methyl-3,4a,5,7,8,8a-hexahydro-2H-pyrido[4,3-b][1,4]oxazin-4-yl]pyridazin-3-yl]-3-methyl-5-(trifluoromethyl)phenol

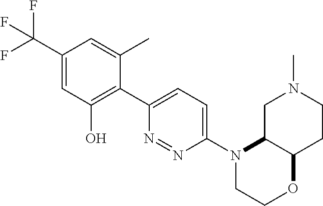

To a solution of 3-methyl-2-[6-[rac-(4aR,8aS)-2,3,4a,5,6,7,8,8a-octahydropyrido[4,3-b][1,4]oxazin-4-yl]pyridazin-3-yl]-5-(trifluoromethyl)phenol (Examples 197-0.62 mmol, 246 mg) in 20 ml of dichloromethane under argon, was added acetic acid (9.36 mmol, 535 μl) and a 37% weight aqueous solution of formaldehyde (3.12 mmol, 253 mg) and the resulting mixture was stirred at rt for 5 min. Then was added portionwise sodium triacetoxyborohydride (1.12 mmol, 238 mg) and the mixture was stirred at rt for 3 h then diluted with 100 ml of dichloromethane. The organic layer was washed with 100 ml of an aqueous saturated sodium bicarbonate solution, dried over Na₂SO₄, filtered and concentrated under reduced pressure. The residue was purified by flash chromatography on silica gel using a gradient of DCM/MeOH/ACN (from 100/0/0 to 80/10/10) to give the title compound as a white solid (111 mg, 44% yield).

Compounds Examples 224 and 225 were synthesized analogously to Example 226 starting from Examples 221 and 195 respectively.

Compound Example 299 were synthesized analogously to Example 226 starting from Intermediate 354.1.

### Example 210: 2-[6-[rac-(4aR,8aS)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol

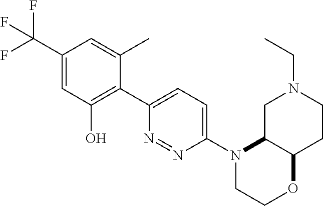

To a solution of 3-methyl-2-[6-[rac-(4aR,8aS)-2,3,4a,5,6,7,8,8a-octahydropyrido[4,3-b][1,4]oxazin-4-yl]pyridazin-3-yl]-5-(trifluoromethyl)phenol (Examples 197-0.51 mmol, 200 mg) in 10 ml of dichloromethane under argon, was added acetic acid (7.6 mmol, 435 μl) and acetaldehyde (2.54 mmol, 142 μl) and the resulting mixture was stirred at rt for 5 min. Then was added portion wise sodium triacetoxyborohydride (0.91 mmol, 193 mg) and the mixture was stirred at rt for 3 h then diluted with 100 ml of dichloromethane. The organic layer was washed with 100 ml of an aqueous saturated sodium bicarbonate solution, dried over Na₂SO₄, filtered and concentrated under reduced pressure. The residue was purified by flash chromatography on silica gel using a gradient of DCM/MeOH/ACN (from 100/0/0 to 80/10/10) to give the title compound as a white solid (155 mg, 72% yield).

Compounds Examples 209 and 223 were synthesized analogously to Examples 210 starting from Examples 195 and 221 respectively.

### Examples 255 and 256: 2-[6-[(4aR,8aS)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol and 2-[6-[(4aS,8aR)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol

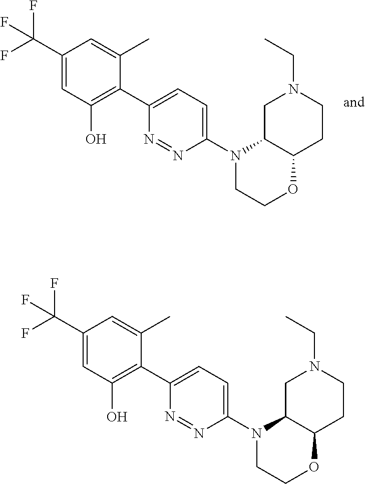 [SMILES: CCN1CC[C@@H]2OCCN(c3ccc(-c4c(C)cc(C(F)(F)F)cc4O)nn3)[C@@H]2C1 ; CCN1CC[C@H]2OCCN(c3ccc(-c4c(C)cc(C(F)(F)F)cc4O)nn3)[C@H]2C1]

2-[6-[rac-(4aR,8aS)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol (Example 210-147 mg) was separated by Supercritical Fluidic Chromatography (SFC—Column: Daicel Chiralpak® AD-H, 250×30 mm I.D., 5 μm particle size; Mobile phase: CO₂/Ethanol (+0.1% Triethylamine) 20%; flow rate: 200 mL/min; outlet pressure: 100 bar; 6 injections; UV detection: 265 nm) to provide 2-[6-[(4aR,8aS)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol, first eluting enantiomer at Rt=6.8 min (58 mg, Example 255, Enantiomer 1) as a white solid, and 2-[6-[(4aS,8aR)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol, second eluting enantiomer at Rt=9.3 min (57 mg, Example 256, Enantiomer 2), as a white solid. The absolute configurations cannot be assigned at this stage.

### Example 214: 1-[rac-(4aS,8aR)-4-[6-[2-hydroxy-6-methyl-4-(trifluoromethyl) phenyl]pyridazin-3-yl]-3,4a,5,7,8,8a-hexahydro-2H-pyrido[4,3-b][1,4]oxazin-6-yl]ethenone

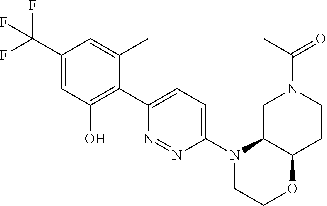 [SMILES: CC(=O)N1CC[C@H]2OCCN(c3ccc(-c4c(C)cc(C(F)(F)F)cc4O)nn3)[C@H]2C1]

### Int 214.1: 1-[rac-(4aS,8aR)-4-[6-[2-hydroxy-6-methyl-4-(trifluoromethyl) phenyl]pyridazin-3-yl]-3,4a,5,7,8,8a-hexahydro-2H-pyrido[4,3-b][1,4]oxazin-6-yl]ethanone and 3-methyl-2-[6-[rac-(4aS,8aR)-6-acetyl-3,4a,5,7,8,8a-hexahydro-2H-pyrido[4,3-b][1,4]oxazin-4-yl]pyridazin-3-yl]-5-(trifluoromethyl) phenyl] acetate

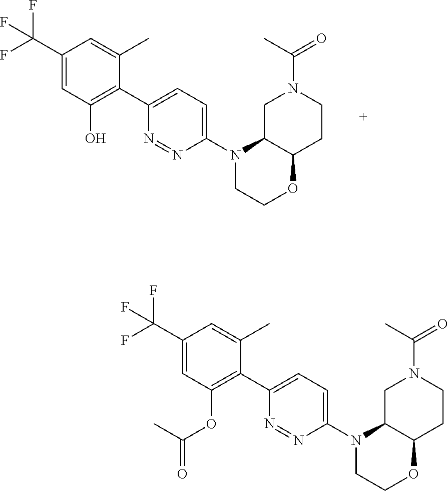 [SMILES: CC(=O)N1CC[C@H]2OCCN(c3ccc(-c4c(C)cc(C(F)(F)F)cc4O)nn3)[C@H]2C1 ; CC(=O)Oc1cc(C(F)(F)F)cc(C)c1-c1ccc(N2CCO[C@@H]3CCN(C(C)=O)C[C@@H]32)nn1]

To a solution of 3-methyl-2-[6-[rac-(4aR,8aS)-2,3,4a,5,6,7,8,8a-octahydropyrido[4,3-b][1,4]oxazin-4-yl]pyridazin-3-yl]-5-(trifluoromethyl)phenol (Examples 197-0.49 mmol, 194 mg) in 8 ml of dichloromethane, was added acetic anhydride (0.74 mmol, 69 μl), N,N-diethylethanamine (0.49 mmol, 67 μl) and N,N-dimethylpyridin-4-amine (0.025 mmol, 3 mg) and the resulting mixture was stirred at rt for 30 min. then diluted with 100 ml of dichloromethane. The organic layer was washed with 100 ml of an aqueous saturated sodium bicarbonate solution, dried over Na₂SO₄, filtered and concentrated under reduced pressure to give a mixture of title compounds as a yellow oil, which was used without further purification in the next step.

### 1-[rac-(4aS,8aR)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone (Example 214)

The residue Int214.1 was taken in a solution of 10 ml of methanol and 1 ml of a TN aqueous sodium hydroxide solution and the mixture was stirred at rt for 30 min then neutralized with a TN aqueous HCl solution until pH=7. The mixture was diluted with 50 ml of dichloromethane and the organic layer was dried over Na₂SO₄, filtered and concentrated under reduced pressure. The residue was purified by flash chromatography on silica gel using a gradient of 0 to 10% of methanol in dichloromethane to give the title compound as a white foam (133 mg, 62% yield).

Compounds Examples 213 and 222 were synthesized analogously to Examples 214 starting from Examples 195 and 221 respectively.

### Examples 262 and 263: 1-[(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone and 1-[(4aS,8aR)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone

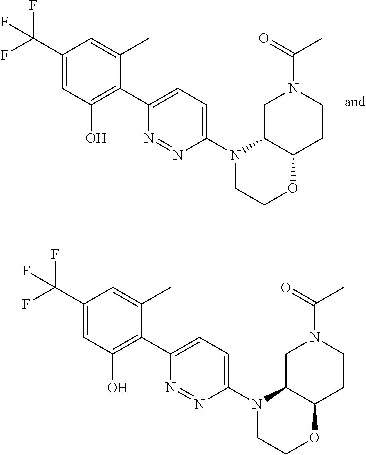 [SMILES: CC(=O)N1CC[C@@H]2OCCN(c3ccc(-c4c(C)cc(C(F)(F)F)cc4O)nn3)[C@@H]2C1 ; CC(=O)N1CC[C@H]2OCCN(c3ccc(-c4c(C)cc(C(F)(F)F)cc4O)nn3)[C@H]2C1]

1-[rac-(4aS,8aR)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone (Example 214-125 mg) was separated by Supercritical Fluidic Chromatography (SFC—Column: Daicel Chiralpak® AD-H, 250×30 mm I.D., 5 μm particle size; Mobile phase: CO₂/Ethanol (+0.1% Triethylamine) 20%; flow rate: 200 mL/min; outlet pressure: 100 bar; 2 injections; UV detection: 265 nm) to provide 1-[(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone, first eluting enantiomer at Rt=7.5 min (47 mg, Example 262, Enantiomer 1) as a white solid, and 1-[(4aS,8aR)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone, second eluting enantiomer at Rt=13.2 min (48 mg, Example 261, Enantiomer 2), as a white solid. The absolute configurations cannot be assigned at this stage.

### Example 268: 2-[6-[(4aR,8aS)-6-methyl-3,4a,5,7,8,8a-hexahydro-2H-pyrido[4,3-b][1,4]oxazin-4-yl]-4-(difluoromethyl)pyridazin-3-yl]-5-(trifluoromethyl)phenol

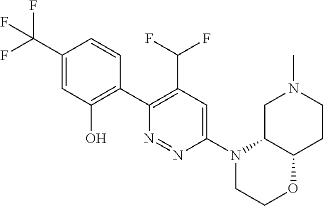 [SMILES: CN1CC[C@@H]2OCCN(c3cc(C(F)F)c(-c4ccc(C(F)(F)F)cc4O)nn3)[C@@H]2C1]

### Int 268.1: tert-butyl (3R,4S)-4-hydroxy-3-[(4-nitrophenyl)sulfonylamino]piperidine-1-carboxylate

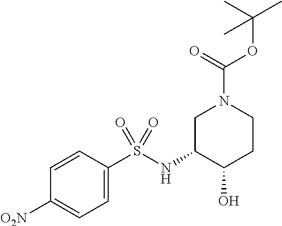 [SMILES: CC(C)(C)OC(=O)N1CC[C@H](O)[C@H](NS(=O)(=O)c2ccc([N+](=O)[O-])cc2)C1]

To a cooled (0-5° C.) solution of tert-butyl (3R,4S)-3-amino-4-hydroxy-piperidine-1-carboxylate (4.62 mmol, 1 g) in 20 ml of dichloromethane was added triethylamine (12.5 mmol, 1.74 ml) and 4-nitrobenzenesulfonyl chloride (5.09 mmol, 1.13 g) and the mixture was stirred for 30 min at rt then diluted with 100 ml of dichloromethane and 100 ml of water. The organic layer was washed with water, dried over Na₂SO₄, filtered and concentrated under reduced pressure to give the title compound (1.5 g, 81% yield) as a yellow solid.

LCMS (Method L): Rt=0.85 min; MS m/z [M+H]⁺ 402.

### Int 268.2: tert-butyl (4aR,8aS)-4-(4-nitrophenyl)sulfonyl-3,4a,5,7,8,8a-hexahydro-2H-pyrido[4,3-b][1,4]oxazine-6-carboxylate

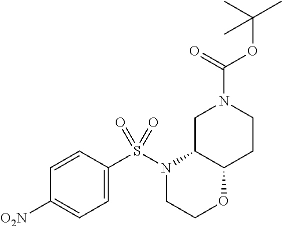 [SMILES: CC(C)(C)OC(=O)N1CC[C@@H]2OCCN(S(=O)(=O)c3ccc([N+](=O)[O-])cc3)[C@@H]2C1]

To a cooled (0-5° C.) solution of tert-butyl (3R,4S)-4-hydroxy-3-[(4-nitrophenyl)sulfonylamino]piperidine-1-carboxylate (3.7 mmol, 1.5 g) in 160 ml of anhydrous dichloromethane under argon was added sodium hydride 60% dispersion in oil (20 mmol, 810 mg) by portions and the mixture was stirred at 0-5° C. for 5 min. Then was added (2-bromoethyl)diphenylsulfonium trifluoromethanesulfonate (10 mmol, 4.4 g) and the mixture was allowed to stir 18 h at RT. Then, triethylamine (9.7 mmol, 1.4 ml) was added and the mixture was stirred at rt for another 2 h then diluted with dichloromethane and water. The aqueous layer was extracted with dichloromethane and the pooled organic layers were dried over Na₂SO₄, filtered and concentrated under reduced pressure. The residue was purified by flash chromatography on silica gel eluting with a gradient of 0 to 40% of EtOAc in cyclohexane to give the title compound (668 mg, 42% yield) as a white foam.

LCMS (Method L): Rt=1.01 min; MS m/z [M+H]⁺ 428.

### Int 268.3: tert-butyl (4aR,8aS)-2,3,4,4a,5,7,8,8a-octahydropyrido[4,3-b][1,4]oxazine-6-carboxylate

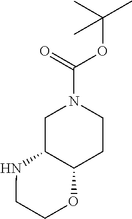 [SMILES: CC(C)(C)OC(=O)N1CC[C@@H]2OCCN[C@@H]2C1]

To a solution of tert-butyl (4aR,8aS)-4-(4-nitrophenyl)sulfonyl-3,4a,5,7,8,8a-hexahydro-2H-pyrido[4,3-b][1,4]oxazine-6-carboxylate (1.56 mmol, 668 mg) in 10 ml of N,N-dimethylformamide was added 2-sulfanylacetic acid (3.75 mmol, 345 mg) and lithium hydroxide; hydrate (8.72 mmol, 366 mg) and the mixture was stirred 18 h at rt then diluted with EtOAc and washed with water (4 times). The organic layer was dried over Na₂SO₄, filtered and concentrated under reduced pressure. The residue was purified by flash chromatography on silica gel eluting with a gradient of 0 to 10% of MeOH in dichloromethane to give the title compound (346 mg, 91% yield) as a colourless oil.

LCMS (Method V): Rt=0.35 min; MS m/z [M+H]⁺ 243

### Int 268.4: tert-butyl (4aR,8aS)-4-[6-chloro-5-(difluoromethyl)pyridazin-3-yl]-3,4a,5,7,8,8a-hexahydro-2H-pyrido[4,3-b][1,4]oxazine-6-carboxylate

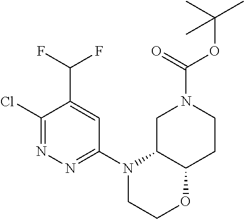 [SMILES: CC(C)(C)OC(=O)N1CC[C@@H]2OCCN(c3cc(C(F)F)c(Cl)nn3)[C@@H]2C1]

A solution of 3,6-dichloro-4-(difluoromethyl)pyridazine (Int 144.1-1.43 mmol, 284 mg), tert-butyl (4aR,8aS)-2,3,4,4a,5,7,8,8a-octahydropyrido[4,3-b][1,4]oxazine-6-carboxylate (1.43 mmol, 346 mg) and triethylamine (3.14 mmol, 438 μl) in 5 ml of n-butanol was stirred at 130° C. for 30 h. The mixture was cooled down, concentrated under reduced pressure and the residue was purified by flash chromatography on silica gel eluting with a gradient of 0 to 20% of EtOAc in cyclohexane to give the title compound (122 mg, 21% yield) as a yellow oil.

LCMS (Method L): Rt=1.03 min; MS m/z [M+H]⁺ 405

### Int 268.5: tert-butyl (4aR,8aS)-4-[5-(difluoromethyl)-6-[2-hydroxy-4-(trifluoromethyl)phenyl]pyridazin-3-yl]-3,4a,5,7,8,8a-hexahydro-2H-pyrido[4,3-b][1,4]oxazine-6-carboxylate

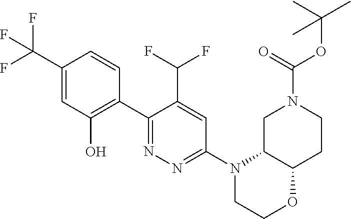 [SMILES: CC(C)(C)OC(=O)N1CC[C@@H]2OCCN(c3cc(C(F)F)c(-c4ccc(C(F)(F)F)cc4O)nn3)[C@@H]2C1]

Following procedure of Example 133, tert-butyl (4aR,8aS)-4-[6-chloro-5-(difluoromethyl)pyridazin-3-yl]-3,4a,5,7,8,8a-hexahydro-2H-pyrido[4,3-b][1,4]oxazine-6-carboxylate (0.3 mmol, 122 mg) gave the title compound (101 mg, 51% yield) as a white solid.

LCMS (Method L): Rt=1.23 min; MS m/z [M+H]⁺ 531

### Int 268.6: 2-[6-[(4aR,8aS)-2,3,4a,5,6,7,8,8a-octahydropyrido[4,3-b][1,4]oxazin-4-yl]-4-(difluoromethyl)pyridazin-3-yl]-5-(trifluoromethyl)phenol

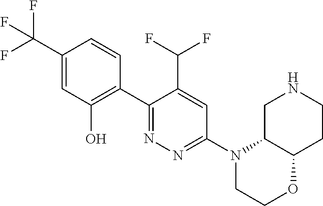 [SMILES: Oc1cc(C(F)(F)F)ccc1-c1nnc(N2CCO[C@H]3CCNC[C@H]32)cc1C(F)F]

To a solution of tert-butyl (4aR,8aS)-4-[5-(difluoromethyl)-6-[2-hydroxy-4-(trifluoromethyl)phenyl]pyridazin-3-yl]-3,4a,5,7,8,8a-hexahydro-2H-pyrido[4,3-b][1,4]oxazine-6-carboxylate (0.19 mmol, 101 mg) in 9 ml of dichloromethane was added 1 ml of trifluoroacetic acid and the mixture was stirred for 2 h at rt then concentrated under reduced pressure. The residue was diluted with 50 ml of dichloromethane and washed with 50 ml of a saturated aqueous sodium bicarbonate solution and 10 ml of water then dried over Na₂SO₄, filtered and concentrated under reduced pressure to give the title compound (79 mg, 96% yield) as a yellow solid.

LCMS (Method L): Rt=0.97 min; MS m/z [M+H]⁺ 431

### 2-[6-[(4aR,8aS)-6-methyl-3,4a,5,7,8,8a-hexahydro-2H-pyrido[4,3-b][1,4]oxazin-4-yl]-4-(difluoromethyl)pyridazin-3-yl]-5-(trifluoromethyl)phenol (Example 268)

Following procedure of Example 226, 2-[6-[(4aR,8aS)-2,3,4a,5,6,7,8,8a-octahydropyrido[4,3-b][1,4]oxazin-4-yl]-4-(difluoromethyl)pyridazin-3-yl]-5-(trifluoromethyl)phenol (0.18 mmol, 79 mg) gave the title compound (29 mg, 36% yield) as a white solid.

### Example 204: 5-cyclopropyl-2-[6-[(2S)-2-(hydroxymethyl)morpholin-4-yl]pyridazin-3-yl]-3-methyl-phenol

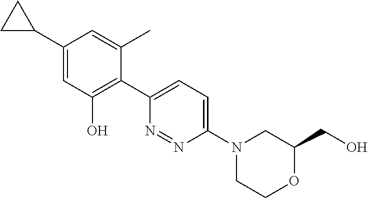 [SMILES: Cc1cc(C2CC2)cc(O)c1-c1ccc(N2CCO[C@H](CO)C2)nn1]

Under Ar, to a solution of 5-cyclopropyl-3-methyl-2-(4,4,5,5-tetramethyl-1,3,2-dioxaborolan-2-yl)phenol (1.37 mmol, 376 mg) and [(2S)-4-(6-chloropyridazin-3-yl)morpholin-2-yl]methanol (Int 8.1-0.65 mmol, 150 mg) in 8 ml of 1,4-dioxane and 2 ml of water was added a 2N sodium carbonate aqueous solution (1.96 mmol, 980 μl) then TETRAKIS(triphenylphosphine)palladium(0) (0.065 mmol, 75 mg). The mixture was stirred at 90° C. for 1 h. After cooling down, the resulting mixture was diluted with dichloromethane and the organic layer was washed successively with a saturated aqueous sodium bicarbonate solution and brine. The organic layer was dried over Na₂SO₄, filtered and concentrated under reduced pressure. The residue was purified by flash chromatography on silica gel using a gradient of DCM/MeOH/ACN (from 100/0/0 to 90/5/5) to give a yellow solid residue which was again purified by prep-HPLC (column: Sunfire prep C18 OBD; 250*50 mm*10 μm; mobile phase: [water (TFA 10⁻² N)-ACN]; B %: 10%-100% in 35 min. at 50 ml/min). The pure fractions were diluted with 10 ml of dichloromethane and washed with 2 ml of a saturated aqueous sodium bicarbonate solution. The organic layer was dried over Na₂SO₄, filtered and concentrated under reduced pressure. The residue was triturated in 4 ml of diethylether/pentane: 50/50 to give after filtration the title compound as a white solid (20 mg, 9% yield).

Compound Example 274 was synthesized analogously to Example 204.

### Examples 287 and 288: (3R)-3-(hydroxymethyl)-1-[6-[2-hydroxy-6-methyl-4-(trifluoromethyl)phenyl]pyridazin-3-yl]piperidin-2-one and (3S)-3-(hydroxymethyl)-1-[6-[2-hydroxy-6-methyl-4-(trifluoromethyl)phenyl]pyridazin-3-yl]piperidin-2-one

The 2 enantiomers of compound 3-(hydroxymethyl)-1-[6-[2-hydroxy-6-methyl-4-(trifluoromethyl)phenyl]pyridazin-3-yl]piperidin-2-one (Example 273, 420 mg) were separated by semi-preparative chiral normal phase chromatography (Column: Chiralpak® AD, 350×76.5 mm, 20 μm particle size; Mobile phase: Heptane 50/EtOH 50)+0.1% TEA; Flow rate: 400 ml/min) to provide the 2 enantiomers, first one eluting at Rt=5.5 min (180 mg, Example 287) as a white solid, and second one eluting at Rt=8.5 min (181 mg, Example 288) as a white solid. The absolute configurations cannot be assigned at this stage.

Compounds Examples 292 and 293 were synthesized and separated enantiomerically analogously to Examples 287 and 288.

### Examples 331 and 332: 3-methyl-2-[6-[(2R)-2-[2-(methylamino)ethyl]morpholin-4-yl]pyridazin-3-yl]-5-(trifluoromethyl)phenol and 3-methyl-2-[6-[(2S)-2-[2-(methylamino)ethyl]morpholin-4-yl]pyridazin-3-yl]-5-(trifluoromethyl)phenol

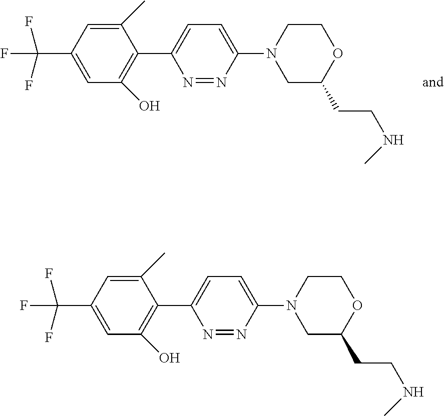 [SMILES: CNCC[C@@H]1CN(c2ccc(-c3c(C)cc(C(F)(F)F)cc3O)nn2)CCO1 ; CNCC[C@H]1CN(c2ccc(-c3c(C)cc(C(F)(F)F)cc3O)nn2)CCO1]

### Int 331.1: tert-butyl N-[2-[4-(6-chloropyridazin-3-yl)morpholin-2-yl]ethyl]carbamate

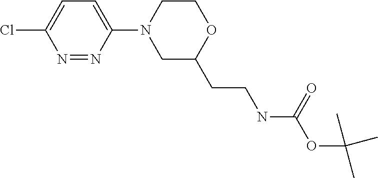 [SMILES: CC(C)(C)OC(=O)NCCC1CN(c2ccc(Cl)nn2)CCO1]

To a solution of 3,6-dichloropyridazine (150 mg, 1.0069 mmol) and tert-butyl N-(2-morpholin-2-ylethyl)carbamate in 3 ml of 1-Butanol was added Triethylamine (0.309 ml, 2.2151 mmol). The mixture was heated at 130° C. for 2 h30. After cooling down to rt, the reaction mixture was diluted with AcOEt, washed with water and brine, dried over MgSO₄ and concentrated under reduced pressure. The residue was purified by flash chromatography on silica gel using Hept/AcOEt (from 100/0 to 50/50) to give the title compound as a white solid (253 mg, 73% yield).

LCMS (Method L): Rt=0.84; MS m/z [M+H]⁺=343

### Int 331.2: tert-butyl N-[2-[4-(6-chloropyridazin-3-yl)morpholin-2-yl]ethyl]-N-methyl-carbamate

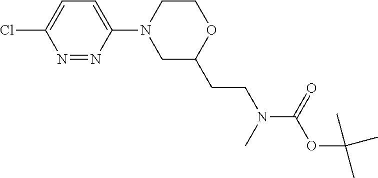 [SMILES: CN(CCC1CN(c2ccc(Cl)nn2)CCO1)C(=O)OC(C)(C)C]

Solution of tert-butyl N-[2-[4-(6-chloropyridazin-3-yl)morpholin-2-yl]ethyl]carbamate (253 mg, 0.738 mmol) in 3 ml of THF was cooled with an ice bath. Sodium hydride (33 mg, 0.8118 mmol, 60 mass %) was added and the resulting mixture was stirred for 20 min. Then Iodomethane (0.100 ml, 1.4718 mmol) was added. After 2 h, additional Sodium hydride (10 mg, 0.2500 mmol, 60 mass %) was added, and 10 min after 2 drops of Iodomethane. The mixture was stirred for 30 min. The reaction mixture was diluted with AcOEt, washed with water and brine, dried over MgSO₄ and concentrated under reduced pressure to give the title compound as a yellow oil (258 mg, 97% yield).

LCMS (Method L): Rt=0.94; MS m/z [M+H]+=357

### Int 331.3: tert-butyl N-[2-[4-[6-[2-hydroxy-6-methyl-4-(trifluoromethyl) phenyl]pyridazin-3-yl]morpholin-2-yl]ethyl]-N-methyl-carbamate

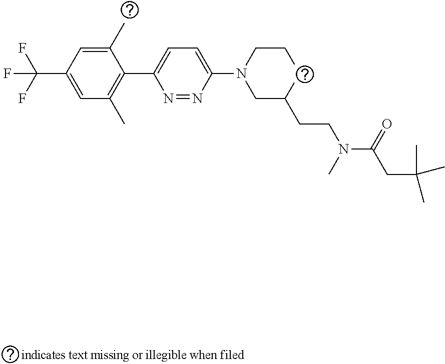 [SMILES: Cc1cc(C(F)(F)F)cc(C)c1-c1ccc(N2CCCC(CCN(C)C(=O)CC(C)(C)C)C2)nn1]

The reaction was carried out in two vials in parallel.

In a microwave vial, under Ar, to a solution of tert-butyl N-[2-[4-(6-chloropyridazin-3-yl)morpholin-2-yl]ethyl]-N-methyl-carbamate (258 mg, 0.723 mmol), 3-methyl-2-(4,4,5,5-tetramethyl-1,3,2-dioxaborolan-2-yl)-5-(trifluoromethyl)phenol (262 mg, 0.8676 mmol), carbonate sodium (192 mg, 1.8075 mmol) in 8 ml of 1,2-Dimethoxyethane and 2 ml of water was added tetrakis(triphenylphosphine)palladium(0) (42 mg; 0.0361 mmol). This procedure was repeated in a second vial. The mixture was heated at 100° C. under microwave irradiation for 1 h30. After cooling down to rt, the mixture of the two vials were joined together, diluted with AcOEt, washed with water and brine, dried over MgSO₄, concentrated under reduced pressure. The residue was purified by flash chromatography on silica gel using DCM/MeOH (from 100/0 to 97/2) to give the title compound as an oil (140 mg, 39% yield).

LCMS (Method L): Rt=1.03; MS m/z [M+H]⁺=497

### 3-methyl-2-[6-[(2R)-2-[2-(methylamino)ethyl]morpholin-4-yl]pyridazin-3-yl]-5-(trifluoromethyl)phenol and 3-methyl-2-[6-[(2S)-2-[2-(methylamino)ethyl]morpholin-4-yl]pyridazin-3-yl]-5-(trifluoromethyl)phenol (Examples 331 and 332)

At 5° C., to a solution of tert-butyl N-[2-[4-[6-[2-hydroxy-6-methyl-4-(trifluoromethyl)phenyl]pyridazin-3-yl]morpholin-2-yl]ethyl]-N-methyl-carbamate (140 mg, 0.282 mmol) in 6 ml of DCM was add 0.7 ml of a solution of HCl 4N in dioxane. After stirring 2 h at rt, the reaction was concentrated under reduce pressure. The residue was precipitated in Et₂O, filtered and washed with acetone to give 3-methyl-2-[6-[2-[2-(methylamino) ethyl]morpholin-4-yl]pyridazin-3-yl]-5-(trifluoromethyl)phenol as a white solid (60 mg, 54% yield). The 2 enantiomers of compound were separated by chiral phase LC (Column Amylosel 5 μm, 250×30 mm, Flow rate 40 ml/min, eluent n-heptane/ethanol (85/15)+0.1% TEA, detection 265 nm) to give both enantiomers, first eluting Rt=28 mn, Example 331 (11.3 mg, 10% yield) and second eluting Rt=41 mn, Example 332 (16.8 mg, 15% yield). The absolute configurations cannot be assigned at this stage.

### Examples 283 and 284: 2-[6-[(2R)-2-(fluoromethyl)morpholin-4-yl]pyridazin-3-yl]-3-methyl-5-(trifluoromethyl)phenol and 2-[6-[(2S)-2-(fluoromethyl)morpholin-4-yl]pyridazin-3-yl]-3-methyl-5-(trifluoromethyl)phenol

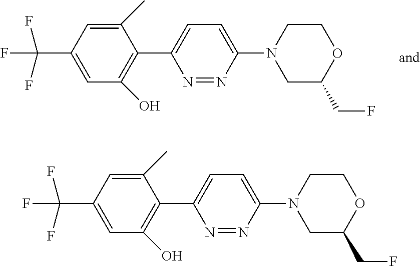 [SMILES: Cc1cc(C(F)(F)F)cc(O)c1-c1ccc(N2CCO[C@H](CF)C2)nn1 ; Cc1cc(C(F)(F)F)cc(O)c1-c1ccc(N2CCO[C@@H](CF)C2)nn1]

2-[6-[2-(fluoromethyl)morpholin-4-yl]pyridazin-3-yl]-3-methyl-5-(trifluoromethyl)phenol (22 mg), obtained similarly to Example 114 was purified by preparative chiral SFC similarly to Examples 237 and 238 using a chiralcel OJ-H column, to give (7 mg, 22%) of a first enantiomer and (6 mg, 19%) of the second one. The absolute configurations cannot be assigned at this stage.

Compounds examples 338 and 339 were enantiomerically separated, analogously to examples 283 and 284 using a Chiralpak® IG column, from a racemic mixture which one was synthesized similarly to Example 114.

### Example 302: 2-[6-[(2R,6R)-2-(hydroxymethyl)-6-methyl-morpholin-4-yl]pyridazin-3-yl]-3-methyl-5-(trifluoromethyl)phenol

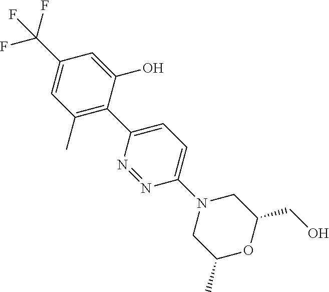 [SMILES: Cc1cc(C(F)(F)F)cc(O)c1-c1ccc(N2C[C@H](CO)O[C@H](C)C2)nn1]

### Int 302.1: [(2R,6R)-4-(6-chloropyridazin-3-yl)-6-methyl-morpholin-2-yl]methanol

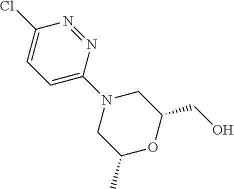 [SMILES: C[C@@H]1CN(c2ccc(Cl)nn2)C[C@H](CO)O1]

In a microwave vial, under Ar, to a solution of 3,6-dichloropyridazine (0,651 mmol, 100 mg) in 5 ml of 1-butanol was added [(2R,6R)-6-methylmorpholin-2-yl]methanol hydrochloride (0.651 mmol, 114 mg) and DIPEA (1.30 mmol, 0.229 mL). The vial was sealed, and the mixture was heated at 130° C. under microwave irradiation for 1 h. After cooling down to rt, the mixture was concentrated under reduced pressure to give the title compound, as an oil (312 mg, 100% yield), which was used in the next step without further purification.

LCMS (Method H): Rt=0.94 min; MS m/z [M+H]⁺=244

### 2-[6-[(2R,6R)-2-(hydroxymethyl)-6-methyl-morpholin-4-yl]pyridazin-3-yl]-3-methyl-5-(trifluoromethyl)phenol (Example 302)

In a microwave vial, under Ar, to a solution of 3-methyl-2-(4,4,5,5-tetramethyl-1,3,2-dioxaborolan-2-yl)-5-(trifluoromethyl)phenol (0.846 mmol, 256 mg) and [(2R,6R)-4-(6-chloropyridazin-3-yl)-6-methyl-morpholin-2-yl]methanol (0.651 mmol, 159 mg) in 4.1 ml of dioxane were added sodium carbonate (1.950 mmol, 162 mg) and 1.02 ml of water. The solution was purged with Ar, then Pd(PPh₃)₄ (32.6 μmol, 38 mg) was added. The vial was sealed, and the mixture was heated at 110° C. under microwave irradiation for 1 h. After cooling down to rt, the reaction mixture was filtered. DCM was added the resulting solution was washed with water (3 times), and brine. The organic layer was dried over MgSO₄, filtered and concentrated under reduced pressure. The residue was purified by flash chromatography on silica gel using DCM/MeOH/ACN (from 100/0/0 to 94/3/3). The solid obtained was triturated in pentane then filtered off and dried to give the title compound as a white solid (80.4 mg, 32% yield).

Compound Examples 296, 297, 359, 365 and 390 were synthesized analogously to Example 302.

### Example 303: 3,5-dimethyl-2-[6-[(2S)-2-(sulfanylmethyl)morpholin-4-yl]pyridazin-3-yl]phenol

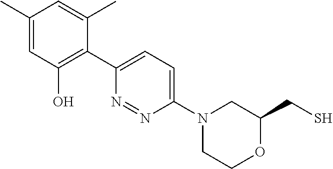 [SMILES: Cc1cc(C)c(-c2ccc(N3CCO[C@H](CS)C3)nn2)c(O)c1]

### Int 303.1: (2S)-4-(6-chloropyridazin-3-yl)-2-(tritylsulfanylmethyl)morpholine

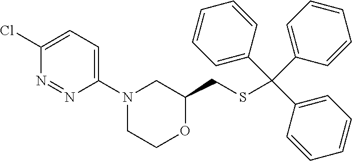 [SMILES: Clc1ccc(N2CCO[C@H](CSC(c3ccccc3)(c3ccccc3)c3ccccc3)C2)nn1]

Under Ar, to a solution of [(2S)-4-(6-chloropyridazin-3-yl)morpholin-2-yl]methanol (Int 8.1-0.94 mmol, 217 mg) and triphenylmethanethiol (2.46 mmol, 679 mg) in 10 ml of THF was added dropwise tributylphosphane (1.98 mmol, 0.51 ml). The mixture was stirred at rt for 10 min. and then was added 1,1′-(azodicarbonyl)dipiperidine (ADDP—1.98 mmol, 501 mg) in one portion. The mixture was stirred for another 5 min then diluted with 5 ml of pentane and filtered off. The white precipitate was washed with 3 ml of pentane and the filtrate was concentrated under reduced pressure. The residue was purified by flash chromatography on silica gel eluting with a gradient of 0 to 5% of EtOAc in dichloromethane to give the title compound (628 mg, 136% yield) as a yellow oil containing by-products and used as it for the next step.

LCMS (Method L): Rt=1.21; MS m/z [M+H]⁺=488

### Int 303.2: 3,5-dimethyl-2-[6-[(2S)-2-(tritylsulfanylmethyl)morpholin-4-yl]pyridazin-3-yl]phenol

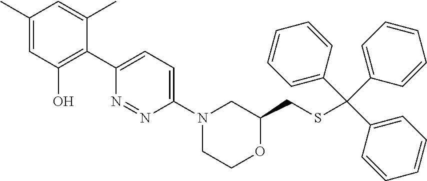 [SMILES: Cc1cc(C)c(-c2ccc(N3CCO[C@H](CSC(c4ccccc4)(c4ccccc4)c4ccccc4)C3)nn2)c(O)c1]

Under Ar, to a solution of (2-hydroxy-4,6-dimethyl-phenyl)boronic acid (0.57 mmol, 95 mg) and (2S)-4-(6-chloropyridazin-3-yl)-2-(tritylsulfanylmethyl)morpholine (Int 303.1-0.38 mmol, 186 mg) in 5 ml of 1,4-dioxane was added a 2N sodium carbonate aqueous solution (1.03 mmol, 514 μl) then Tetrakis(triphenylphosphine)palladium(0) (0.019 mmol, 22 mg). The mixture was stirred at 100° C. for 2 h. After cooling down, the mixture was diluted with 20 ml of EtOAc and the organic layer was washed successively with water and brine then dried over Na₂SO₄, filtered and concentrated under reduced pressure. The residue was purified by flash chromatography on silica gel eluting with a gradient of 0 to 5% of EtOAc in DCM to give the title compound (113 mg, 52% yield—70% for the 2 steps) as a yellow oil.

LCMS (Method L) Rt=1.07; MS m/z [M+H]⁺=574

### 3,5-dimethyl-2-[6-[(2S)-2-(sulfanylmethyl)morpholin-4-yl]pyridazin-3-yl]phenol (Example 303)

To a cooled (0° C.) solution of 3,5-dimethyl-2-[6-[(2S)-2-(tritylsulfanylmethyl)morpholin-4-yl]pyridazin-3-yl]phenol (Int 303.2-0.19 mmol, 111 mg) in 2 ml of DCM were added triisopropylsilane (0.93 mmol, 190 μl) and trifluoroacetic acid (7.74 mmol, 575 μl) and the mixture was stirred at rt for 10 min then concentrated under reduced pressure. The residue was co-evaporated twice with 5 ml of toluene then purified by flash chromatography on silica gel eluting with a gradient of 0 to 5% of MeOH in DCM to give the title compound (60 mg, 94% yield) as a yellow gum.

Compound Example 305 was synthesized analogously to Example 303.

### Examples 327 and 328: 2-[(2R)-4-[6-[2-hydroxy-6-methyl-4-(trifluoromethyl)phenyl]pyridazin-3-yl]morpholin-2-yl]acetonitrile and 2-[(2S)-4-[6-[2-hydroxy-6-methyl-4-(trifluoromethyl)phenyl]pyridazin-3-yl]morpholin-2-yl]acetonitrile

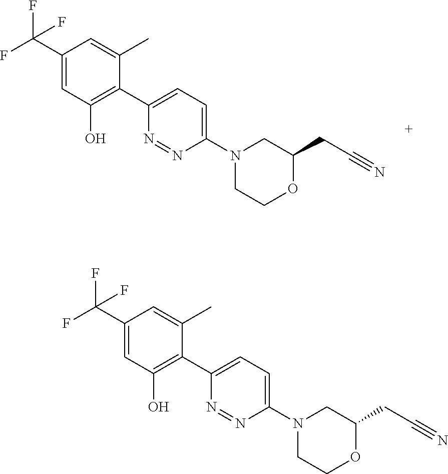 [SMILES: Cc1cc(C(F)(F)F)cc(O)c1-c1ccc(N2CCO[C@H](CC#N)C2)nn1 ; Cc1cc(C(F)(F)F)cc(O)c1-c1ccc(N2CCO[C@@H](CC#N)C2)nn1]

### Int 327.1: (rac)-2-morpholin-2-ylacetonitrile;2,2,2-trifluoroacetic acid

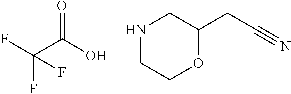 [SMILES: O=C(O)C(F)(F)F ; N#CCC1CNCCO1]

At 0° C., to a solution of (rac)-tert-butyl 2-(cyanomethyl)morpholine-4-carboxylate (4.42 mmol, 1 g) in 40 ml of DCM was added trifluoroacetic acid (44.2 mmol, 3.28 ml) and the mixture was stirred at rt for 1 h, then concentrated under reduced pressure. The residue was co-evaporated with 3×50 ml of toluene, then triturated in diethylether to give the title compound (1.12 g, 106% yield) as a white solid used without further purification in the next step.

### Int 327.2: (rac)-2-[4-(6-chloropyridazin-3-yl)morpholin-2-yl]acetonitrile

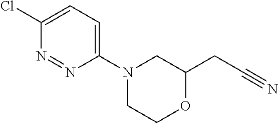 [SMILES: N#CCC1CN(c2ccc(Cl)nn2)CCO1]

A solution of 3,6-dichloropyridazine (3.89 mmol, 580 mg), (rac)-2-morpholin-2-ylacetonitrile;2,2,2-trifluoroacetic acid (4.67 mmol, 1.12 g) and triethylamine (8.56 mmol, 1.19 ml) in 35 ml of 1-butanol was stirred at 120° C. for 45 h. After cooling down to rt, the mixture was concentrated under reduced pressure. The residue was diluted with 50 ml of DCM then washed successively with 20 ml of a saturated aqueous sodium bicarbonate solution and 10 ml of brine. The organic layer was dried over Na₂SO₄, filtered and concentrated under reduced pressure. The residue was purified by flash chromatography on silica gel using a gradient of DCM/MeOH/ACN (from 100/0/0 to 96/2/2) to give the title compound as a light brown solid (198 mg, 21% yield).

LCMS (Method Q): Rt=0.51; MS m/z [M+H]⁺=239

### 2-[(2R)-4-[6-[2-hydroxy-6-methyl-4-(trifluoromethyl)phenyl]pyridazin-3-yl]morpholin-2-yl]acetonitrile and 2-[(2S)-4-[6-[2-hydroxy-6-methyl-4-(trifluoromethyl)phenyl] pyridazin-3-yl]morpholin-2-yl]acetonitrile (Example 327 and Example 328)

Under Ar, to a solution of 3-methyl-2-(4,4,5,5-tetramethyl-1,3,2-dioxaborolan-2-yl)-5-(trifluoromethyl)phenol (1.24 mmol, 376 mg) and (rac)-2-[4-(6-chloropyridazin-3-yl)morpholin-2-yl]acetonitrile (Int 327.2-0.83 mmol, 198 mg) in 5 ml of 1,4-dioxane was added a 2N sodium carbonate aqueous solution (2.24 mmol, 1.12 ml) then tetrakis(triphenylphosphine)palladium(0) (0.042 mmol, 48 mg). The mixture was stirred at 100° C. for 1 h. After cooling down, the mixture was diluted with 40 ml of EtOAc and the organic layer was washed successively with water and brine then dried over Na₂SO₄, filtered and concentrated under reduced pressure. The residue was purified by flash chromatography on silica gel eluting with a gradient of 0 to 50% of EtOAc in cyclohexane to give (rac)-2-[4-[6-[2-hydroxy-6-methyl-4-(trifluoromethyl)phenyl]pyridazin-3-yl]morpholin-2-yl]acetonitrile (218 mg, 69% yield) as a white solid. This racemate was separated by SFC (Column: Daicel Chiralpak® IG, 250×30 mm I.D., 5 μm particle size; Mobile phase: Eluent B: MeOH (0.1% TEA), Isocratic elution: 18% Phase B in Supercritical CO₂; Flow rate: 120 ml/min; (UV detection: 265 nm; 30 injections) to provide the 2 enantiomers, first one eluting at Rt=8.06 min (21 mg, Example 327, Enantiomer 1) as a white solid, and second one eluting at Rt=9.43 min (15 mg, Example 328, Enantiomer 2) as a white solid. The absolute configurations cannot be assigned.

### Example 298: 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-5-(trifluoromethyl)phenol

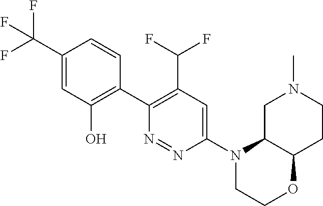 [SMILES: CN1CC[C@H]2OCCN(c3cc(C(F)F)c(-c4ccc(C(F)(F)F)cc4O)nn3)[C@H]2C1]

### Int 298.1: tert-butyl (3S,4R)-4-hydroxy-3-[(4-nitrophenyl)sulfonylamino]piperidine-1-carboxylate

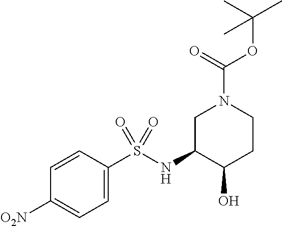 [SMILES: CC(C)(C)OC(=O)N1CC[C@@H](O)[C@@H](NS(=O)(=O)c2ccc([N+](=O)[O-])cc2)C1]

To a cooled solution (0-5° C.) of tert-butyl (3S,4R)-3-amino-4-hydroxy-piperidine-1-carboxylate (46.2 mmol, 10 g) in 300 ml of dichloromethane was added triethylamine (125 mmol, 17.4 ml) and 4-nitrobenzenesulfonyl chloride (50.9 mmol, 11.3 g) and the mixture was stirred for 30 min at rt. The mixture was then diluted with 600 ml of diethylether and 500 ml of water. The organic layer was washed with 250 ml of water, 250 ml of a 5% aqueous citric acid solution and 100 ml of brine (twice), dried over Na₂SO₄, filtered and concentrated under reduced pressure to give the title compound (19.6 g, 100% yield) as a pale yellow solid.

LCMS (Method V): Rt=0.96; MS m/z[M+H]⁺=400

### Int 298.2: tert-butyl (4aS,8aR)-4-(4-nitrophenyl)sulfonyl-3,4a,5,7,8,8a-hexahydro-2H-pyrido[4,3-b][1,4]oxazine-6-carboxylate

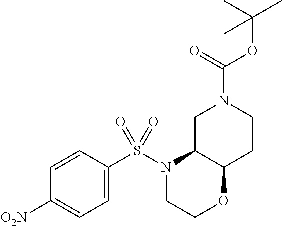 [SMILES: CC(C)(C)OC(=O)N1CC[C@H]2OCCN(S(=O)(=O)c3ccc([N+](=O)[O-])cc3)[C@H]2C1]

Under Ar, a suspension of tert-butyl (3S,4R)-4-hydroxy-3-[(4-nitrophenyl)sulfonylamino]piperidine-1-carboxylate (24.9 mmol, 10 g), potassium carbonate (100 mmol, 13.8 g) and 1,2-dibromoethane (249 mmol, 21.8 ml) in 500 ml of acetonitrile was stirred at reflux for 30 h. After cooling down to rt, the mixture was filtered, the cake rinsed with 50 ml of acetonitrile and the filtrate concentrated under reduced pressure. The residue was purified by flash chromatography on silica gel eluting with a gradient of 0 to 100% of EtOAc in cyclohexane to give the title compound (7.35 g, 73% yield on the 2 steps) as a yellow foam.

LCMS (Method V): Rt=1.18; MS m/z [M+H]⁺=428

### Int 298.3: tert-butyl (4aS,8aR)-2,3,4,4a,5,7,8,8a-octahydropyrido[4,3-b][1,4]oxazine-6-carboxylate

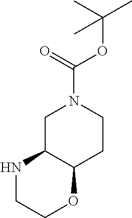 [SMILES: CC(C)(C)OC(=O)N1CC[C@H]2OCCN[C@H]2C1]

To a solution of tert-butyl (4aS,8aR)-4-(4-nitrophenyl)sulfonyl-3,4a,5,7,8,8a-hexahydro-2H-pyrido[4,3-b][1,4]oxazine-6-carboxylate (34.4 mmol, 14.7 g) and 2-sulfanylacetic acid (82.5 mmol, 5.76 ml) in 200 ml of N,N-dimethylformamide were added lithium hydroxide hydrate (193 mmol, 8.08 g) and the mixture was stirred at rt for 18 h. The mixture was then diluted with 1 L of EtOAc and washed successively with 500 ml of water, 500 ml then 250 ml of a 0.1 N sodium hydroxide aqueous solution. The aqueous layers were pooled and successively extracted with 2×500 ml of EtOAc and 2×250 ml of a mixture of chloroform/isopropanol: 90/10. All the organic layers were pooled and washed with 100 ml of a 0.1 N sodium hydroxide aqueous solution, then dried over Na₂SO₄, filtered and concentrated under reduced pressure at 70° C. to give the title compound (7.22 g, 87% yield) as a yellow oil.

LCMS (Method W): Rt=0.42; MS m/z [M+H]⁺=243

### Int 298.4: tert-butyl (4aS,8aR)-4-[6-chloro-5-(difluoromethyl)pyridazin-3-yl]-3,4a,5,7,8,8a-hexahydro-2H-pyrido[4,3-b][1,4]oxazine-6-carboxylate and Int 291.1: tert-butyl (4aS,8aR)-4-[6-chloro-4-(difluoromethyl)pyridazin-3-yl]-3,4a,5,7,8,8a-hexahydro-2H-pyrido[4,3-b][1,4]oxazine-6-carboxylate

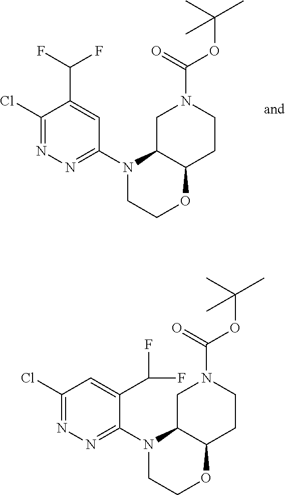 [SMILES: CC(C)(C)OC(=O)N1CC[C@H]2OCCN(c3cc(C(F)F)c(Cl)nn3)[C@H]2C1 ; CC(C)(C)OC(=O)N1CC[C@H]2OCCN(c3nnc(Cl)cc3C(F)F)[C@H]2C1]

To a mixture of tert-butyl (4aS,8aR)-2,3,4,4a,5,7,8,8a-octahydropyrido[4,3-b][1,4]oxazine-6-carboxylate (9.90 mmol, 2.3 g) and 3,6-dichloro-4-(difluoromethyl)pyridazine (Int 144.1, 14.9 mmol, 3 g) was added triethylamine (19.8 mmol, 2.8 ml). The mixture was stirred at 140° C. for 10 min (while removing the excess of triethylamine under a flow of argon), then for another 30 min. The neat solution was then cooled to rt, diluted with 50 ml of DCM and purified by flash chromatography on silica gel eluting with a gradient of 0 to 100% of a mixture of EtOAc/MeOH: 99/1 in cyclohexane to give successively tert-butyl (4aS,8aR)-4-[6-chloro-4-(difluoromethyl)pyridazin-3-yl]-3,4a,5,7,8,8a-hexahydro-2H-pyrido[4,3-b][1,4]oxazine-6-carboxylate—Int 291.1 (1.17 g, 29% yield) and tert-butyl (4aS,8aR)-4-[6-chloro-5-(difluoromethyl)pyridazin-3-yl]-3,4a,5,7,8,8a-hexahydro-2H-pyrido[4,3-b][1,4]oxazine-6-carboxylate—Int 298.4 (1.71 g, 43% yield) as yellow oils.

### LCMS (Method V):

Int 291.1: Rt=1.17; MS m/z [M+H]⁺=405

Int 298.4: Rt=1.15; MS m/z [M+H]⁺=405

### Int 298.5: (4aS,8aR)-4-[6-chloro-5-(difluoromethyl)pyridazin-3-yl]-2,3,4a,5,6,7,8,8a-octahydropyrido[4,3-b][1,4]oxazine

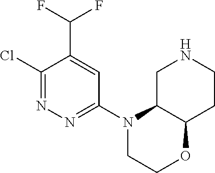 [SMILES: FC(F)c1cc(N2CCO[C@@H]3CCNC[C@@H]32)nnc1Cl]

To a solution of tert-butyl (4aS,8aR)-4-[6-chloro-5-(difluoromethyl)pyridazin-3-yl]-3,4a,5,7,8,8a-hexahydro-2H-pyrido[4,3-b][1,4]oxazine-6-carboxylate (1.31 mmol, 530 mg) in 45 ml of dichloromethane were added 5 ml of trifluoroacetic acid and the mixture was stirred for 2 h at RT then concentrated under reduced pressure. The residue was diluted with 50 ml of dichloromethane and washed with 50 ml of a saturated aqueous sodium bicarbonate solution and 10 ml of water then dried over Na₂SO₄, filtered and concentrated under reduced pressure to give the title compound (400 mg, 100% yield) as a yellow solid.

LCMS (Method V): Rt=0.39; MS m/z [M+H]⁺=305

### Int 298.6: (4aS,8aR)-4-[6-chloro-5-(difluoromethyl)pyridazin-3-yl]-6-methyl-3,4a,5,7,8,8a-hexahydro-2H-pyrido[4,3-b][1,4]oxazine

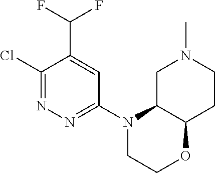 [SMILES: CN1CC[C@H]2OCCN(c3cc(C(F)F)c(Cl)nn3)[C@H]2C1]

To a solution of (4aS,8aR)-4-[6-chloro-5-(difluoromethyl)pyridazin-3-yl]-2,3,4a,5,6,7,8,8a-octahydropyrido[4,3-b][1,4]oxazine (1.91 mmol, 581 mg) and a 37% weight formaldehyde aqueous solution (9.53 mmol, 774 mg) in 40 ml of DCM were added 1.63 ml of acetic acid and the mixture was stirred for 10 min at rt. Then was added sodium triacetoxyborohydride (3.43 mmol, 727 mg) and the mixture was stirred 2 h at rt, then diluted with 100 ml of DCM and washed with 100 ml of a saturated aqueous sodium bicarbonate solution. The organic layer was dried over Na₂SO₄, filtered and concentrated under reduced pressure. The residue was purified by flash chromatography on silica gel using a gradient of DCM/MeOH/ACN (from 100/0/0 to 95/2.5/2.5) to give the title compound as a yellow oil (401 mg, 66% yield).

LCMS (Method V): Rt=0.41; MS m/z [M+H]⁺=319

### 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-5-(trifluoromethyl)phenol (Example 298)

Under Ar, to a solution of [2-hydroxy-4-(trifluoromethyl)phenyl]boronic acid (0.47 mmol, 97 mg) and (4aS,8aR)-4-[6-chloro-5-(difluoromethyl)pyridazin-3-yl]-6-methyl-3,4a,5,7,8,8a-hexahydro-2H-pyrido[4,3-b][1,4]oxazine (Int 298.6-0.31 mmol, 100 mg) in 5 ml of 1,4-dioxane was added a 2N sodium carbonate aqueous solution (0.85 mmol, 424 μl) then Tetrakis(triphenylphosphine)palladium(0) (0.016 mmol, 18 mg). The mixture was stirred at 100° C. for 1 h. After cooling down to rt, the mixture was diluted with 20 ml of EtOAc and 20 ml of diethylether and the organic layer was washed successively with 2×15 ml of a saturated aqueous sodium bicarbonate solution and 15 ml of water. The organic layer was dried over Na₂SO₄, filtered and concentrated under reduced pressure. The residue was purified by flash chromatography on silica gel using a gradient of DCM/MeOH/ACN (from 100/0/0 to 85/7.5/7.5), then repurified by prep-HPLC (column: Sunfire prep C18 OBD; 250*50 mm*10 μm; mobile phase: [aqueous TFA 10⁻² N/ACN] from 10% to 100% ACN in 35 min. at 50 ml/min) to give the title compound as a white solid (45 mg, 32% yield).

### Example 291: 2-[6-[(4aS,8aR)-6-methyl-3,4a,5,7,8,8a-hexahydro-2H-pyrido[4,3-b][1,4]oxazin-4-yl]-5-(difluoromethyl)pyridazin-3-yl]-3,5-dimethyl-phenol

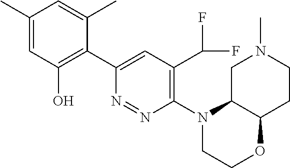 [SMILES: Cc1cc(C)c(-c2cc(C(F)F)c(N3CCO[C@@H]4CCN(C)C[C@@H]43)nn2)c(O)c1]

### Int 291.2: (4aS,8aR)-4-[6-chloro-4-(difluoromethyl)pyridazin-3-yl]-2,3,4a,5,6,7,8,8a-octahydropyrido[4,3-b][1,4]oxazine

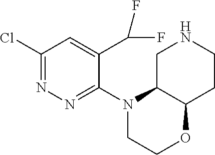 [SMILES: FC(F)c1cc(Cl)nnc1N1CCO[C@@H]2CCNC[C@@H]21]

To a solution of tert-butyl (4aS,8aR)-4-[6-chloro-4-(difluoromethyl)pyridazin-3-yl]-3,4a,5,7,8,8a-hexahydro-2H-pyrido[4,3-b][1,4]oxazine-6-carboxylate (Int 291.1-2.89 mmol, 1.17 g) in 18 ml of dichloromethane were added 2 ml of trifluoroacetic acid and the mixture was stirred for 2 h at rt, then diluted with 50 ml of dichloromethane and washed with a mixture of 20 ml of a saturated aqueous sodium bicarbonate solution and 10 ml of a 2N aqueous Na₂CO₃ solution. The organic layer was dried over Na₂SO₄, filtered and concentrated under reduced pressure to give the title compound (810 mg, 92% yield) as a yellow gum.

LCMS (Method V): Rt=0.39; MS m/z [M+H]⁺=305

### Int 291.3: (4aS,8aR)-4-[6-chloro-4-(difluoromethyl)pyridazin-3-yl]-6-methyl-3,4a,5,7,8,8a-hexahydro-2H-pyrido[4,3-b][1,4]oxazine

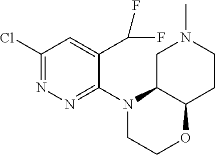 [SMILES: CN1CC[C@H]2OCCN(c3nnc(Cl)cc3C(F)F)[C@H]2C1]

To a solution of (4aS,8aR)-4-[6-chloro-4-(difluoromethyl)pyridazin-3-yl]-2,3,4a,5,6,7,8,8a-octahydropyrido[4,3-b][1,4]oxazine (1.31 mmol, 401 mg) and a 37% weight formaldehyde aqueous solution (6.56 mmol, 533 mg) in 30 ml of DCM were added 1.3 ml of acetic acid and the mixture was stirred for 10 min at RT. Then was added sodium triacetoxyborohydride (2.36 mmol, 501 mg) and the mixture was stirred 1 h at rt, then diluted with 20 ml of DCM and washed with 20 ml of a 2N aqueous Na₂CO₃ solution. The organic layer was dried over Na₂SO₄, filtered and concentrated under reduced pressure. The residue was purified by flash chromatography on silica gel eluting with a gradient of 0 to 10% of MeOH in DCM to give the title compound (342 mg, 82% yield) as a yellow oil.

LCMS (Method V): Rt=0.39; MS m/z [M+H]⁺=319

### 2-[6-[(4aS,8aR)-6-methyl-3,4a,5,7,8,8a-hexahydro-2H-pyrido[4,3-b][1,4]oxazin-4-yl]-5-(difluoromethyl)pyridazin-3-yl]-3,5-dimethyl-phenol (Example 291)

Under Ar, to a solution of (2-hydroxy-4,6-dimethyl-phenyl)boronic acid (0.72 mmol, 119 mg) and (4aS,8aR)-4-[6-chloro-4-(difluoromethyl)pyridazin-3-yl]-6-methyl-3,4a,5,7,8,8a-hexahydro-2H-pyrido[4,3-b][1,4]oxazine (Int 291.3-0.36 mmol, 114 mg) in 4 ml of 1,4-dioxane and 0.75 ml of water was added a 2N sodium carbonate aqueous solution (1.07 mmol, 537 μl) then tetrakis(triphenylphosphine)palladium(0) (0.036 mmol, 41 mg). The mixture was stirred at 100° C. for 2 h30. After cooling down to rt, the mixture was diluted with 20 ml of DCM and 20 ml of saturated aqueous sodium bicarbonate. The organic layer was dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 89/5/5/1) to give the title compound as a white solid (114 mg, 79% yield).

Compound Example 425 was synthesized analogously to Example 291.

### Example 295: 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-5-methyl-phenol

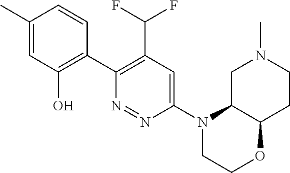 [SMILES: Cc1ccc(-c2nnc(N3CCO[C@@H]4CCN(C)C[C@@H]43)cc2C(F)F)c(O)c1]

Under Ar, to a solution of (2-hydroxy-4-methyl-phenyl)boronic acid (0.47 mmol, 71.5 mg) and (4aS,8aR)-4-[6-chloro-5-(difluoromethyl)pyridazin-3-yl]-6-methyl-3,4a,5,7,8,8a-hexahydro-2H-pyrido[4,3-b][1,4]oxazine (Int 298.6-0.31 mmol, 100 mg) in 8 ml of 1,4-dioxane was added a 2N sodium carbonate aqueous solution (0.85 mmol, 424 μl), then tetrakis(triphenylphosphine)palladium(0) (0.016 mmol, 18 mg). The mixture was stirred at 100° C. for 1 h. After cooling down to rt, the mixture was diluted with 20 ml of EtOAc and 20 ml of diethylether and the organic layer was washed successively with 2×15 ml of a saturated aqueous sodium bicarbonate solution and 15 ml of water. The organic layer was dried over Na₂SO₄, filtered and concentrated under reduced pressure. The residue was purified by flash chromatography on silica gel using a gradient of DCM/MeOH/ACN (from 100/0/0 to 85/7.5/7.5) to give the title compound as a white solid (58 mg, 38% yield).

Compounds Examples 306 and 351 were synthetized analogously to Example 295

### Example 323: 2-[6-[(4aS,8aR)-6-ethyl-3,4a,5,7,8,8a-hexahydro-2H-pyrido[4,3-b][1,4]oxazin-4-yl]-4-(difluoromethyl)pyridazin-3-yl]-5-methyl-phenol

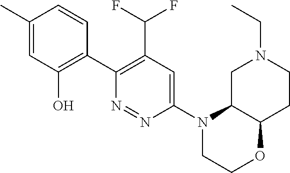 [SMILES: CCN1CC[C@H]2OCCN(c3cc(C(F)F)c(-c4ccc(C)cc4O)nn3)[C@H]2C1]

### Int 323.1: (4aS,8aR)-4-[6-chloro-5-(difluoromethyl)pyridazin-3-yl]-6-ethyl-3,4a,5,7,8,8a-hexahydro-2H-pyrido[4,3-b][1,4]oxazine

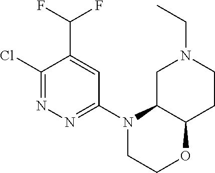 [SMILES: CCN1CC[C@H]2OCCN(c3cc(C(F)F)c(Cl)nn3)[C@H]2C1]

To a solution of (4aS,8aR)-4-[6-chloro-5-(difluoromethyl)pyridazin-3-yl]-2,3,4a,5,6,7,8,8a-octahydropyrido[4,3-b][1,4]oxazine (Int 298.5-0.69 mmol, 211 mg) and acetaldehyde (3.46 mmol, 152 mg) in 20 ml of DCM was added 0.594 ml of acetic acid and the mixture was stirred for 10 min at RT. Then was added sodium triacetoxyborohydride (1.25 mmol, 264 mg) and the mixture was stirred 2 h at rt, then diluted with 50 ml of DCM and washed with 50 ml of a saturated aqueous sodium bicarbonate solution. The organic layer was dried over Na₂SO₄, filtered and concentrated under reduced pressure. The residue was purified by flash chromatography on silica gel using a gradient of DCM/MeOH/ACN (from 100/0/0 to 80/10/10) to give the title compound as a yellow solid (138 mg, 60% yield).

LCMS (Method V): Rt=0.42; MS m/z [M+H]⁺=333

### 2-[6-[(4aS,8aR)-6-ethyl-3,4a,5,7,8,8a-hexahydro-2H-pyrido[4,3-b][1,4]oxazin-4-yl]-4-(difluoromethyl)pyridazin-3-yl]-5-methyl-phenol (Example 323)

Following procedure of Example 298, (4aS,8aR)-4-[6-chloro-5-(difluoromethyl)pyridazin-3-yl]-6-ethyl-3,4a,5,7,8,8a-hexahydro-2H-pyrido[4,3-b][1,4]oxazine (0.41 mmol, 138 mg) gave the title compound as a white solid (73 mg, 43% yield).

### Example 324: 5-[6-[(2S)-2-(hydroxymethyl)morpholin-4-yl]-4-methyl-pyridazin-3-yl]indan-4-ol

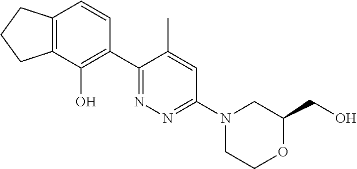 [SMILES: Cc1cc(N2CCO[C@H](CO)C2)nnc1-c1ccc2c(c1O)CCC2]

Under Ar, to a solution of 5-(4,4,5,5-tetramethyl-1,3,2-dioxaborolan-2-yl)indan-4-ol (Int 324.2-0.92 mmol, 276 mg) and [(2S)-4-(6-chloro-5-methyl-pyridazin-3-yl)morpholin-2-yl]methanol (Int 85.1-0.62 mmol, 150 mg) in 5 ml of 1,4-dioxane was added a 2N sodium carbonate aqueous solution (1.66 mmol, 831 μl) then tetrakis(triphenylphosphine)palladium(0) (0.031 mmol, 36 mg). The mixture was stirred at 100° C. for 3 h. After cooling down to rt, the mixture was diluted with 50 ml of EtOAc and 50 ml of diethylether and the organic layer was washed successively with 20 ml of a saturated aqueous sodium bicarbonate solution and 20 ml of water. The organic layer was dried over Na₂SO₄, filtered and concentrated under reduced pressure. The residue was purified by flash chromatography on silica gel eluting with a gradient of 0 to 5% of MeOH in DCM and the solid obtained was triturated in 2 ml of acetonitrile to give the title compound as a white solid (58 mg, 28% yield).

### Example 345: 5-[4-(difluoromethyl)-6-[(2S)-2-(hydroxymethyl)morpholin-4-yl]pyridazin-3-yl]indan-4-ol

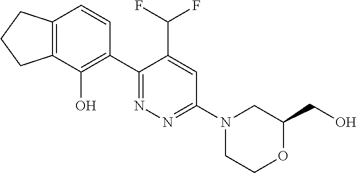 [SMILES: OC[C@@H]1CN(c2cc(C(F)F)c(-c3ccc4c(c3O)CCC4)nn2)CCO1]

Under Ar, to a solution of 5-(4,4,5,5-tetramethyl-1,3,2-dioxaborolan-2-yl)indan-4-ol (Int 324.2-0.86 mmol, 257 mg) and [(2S)-4-[6-chloro-5-(difluoromethyl)pyridazin-3-yl]morpholin-2-yl]methanol (Int 144.2-0.57 mmol, 160 mg) in 5 ml of 1,4-dioxane was added a 2N sodium carbonate aqueous solution (1.54 mmol, 772 μl) then tetrakis(triphenylphosphine)palladium(0) (0.029 mmol, 33 mg). The mixture was stirred at 100° C. for 1 h. After cooling down to rt, the mixture was diluted with 50 ml of EtOAc and 50 ml of diethylether and the organic layer was washed successively with 20 ml of a saturated aqueous sodium bicarbonate solution and 20 ml of water. The organic layer was dried over Na₂SO₄, filtered and concentrated under reduced pressure. The residue was purified by flash chromatography on silica gel eluting with a gradient of 0 to 5% of MeOH in DCM then repurified by prep-HPLC (column: Sunfire prep C18 OBD; 250*50 mm*10 μm; mobile phase: [aqueous TFA 10⁻² N/ACN] from 10% to 100% ACN in 25 min. at 50 ml/min) to give the title compound as a white solid (98 mg, 45% yield).

### Example 350: 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-5-chloro-phenol

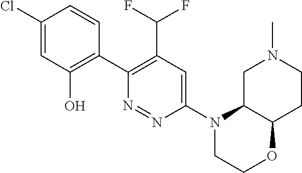 [SMILES: CN1CC[C@H]2OCCN(c3cc(C(F)F)c(-c4ccc(Cl)cc4O)nn3)[C@H]2C1]

Under Ar, to a solution of 5-chloro-2-(4,4,5,5-tetramethyl-1,3,2-dioxaborolan-2-yl)phenol (0.94 mmol, 240 mg) and (4aS,8aR)-4-[6-chloro-5-(difluoromethyl)pyridazin-3-yl]-6-methyl-3,4a,5,7,8,8a-hexahydro-2H-pyrido[4,3-b][1,4]oxazine (Int 298.6-0.63 mmol, 200 mg) in 8 ml of 1,4-dioxane was added a 2N sodium carbonate aqueous solution (1.69 mmol, 847 μl), then tetrakis(triphenylphosphine)palladium(0) (0.031 mmol, 36 mg). The mixture was stirred at 100° C. for 1 h. After cooling down to rt, the mixture was diluted with 20 ml of EtOAc and 20 ml of diethylether and the organic layer was washed successively with 2×15 ml of a saturated aqueous sodium bicarbonate solution and 15 ml of water. The organic layer was dried over Na₂SO₄, filtered and concentrated under reduced pressure. The residue was purified by flash chromatography on silica gel using a gradient of DCM/MeOH/ACN (from 100/0/0 to 85/7.5/7.5), then repurified by prep-HPLC (column: Sunfire prep C18 OBD; 250*50 mm*10 μm; mobile phase: [aqueous TFA 10⁻² N/ACN] from 10% to 100% ACN in 35 min. at 50 ml/min) to give the title compound as a white solid (49 mg, 19% yield).

### Example 386: 1-[(4aS,8aR)-4-[5-(difluoromethyl)-6-[2-hydroxy-4-(trifluoromethyl) phenyl]pyridazin-3-yl]-3,4a,5,7,8,8a-hexahydro-2H-pyrido[4,3-b][1,4]oxazin-6yl]ethanone

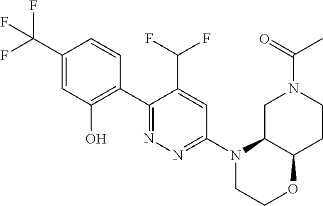 [SMILES: CC(=O)N1CC[C@H]2OCCN(c3cc(C(F)F)c(-c4ccc(C(F)(F)F)cc4O)nn3)[C@H]2C1]

### Int 386.1: 1-[(4aS,8aR)-4-[6-chloro-5-(difluoromethyl)pyridazin-3-yl]-3,4a,5,7,8,8a-hexahydro-2H-pyrido[4,3-b][1,4]oxazin-6-yl]ethanone

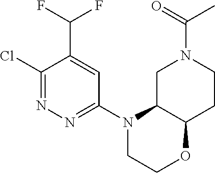 [SMILES: CC(=O)N1CC[C@H]2OCCN(c3cc(C(F)F)c(Cl)nn3)[C@H]2C1]

To a solution of (4aS,8aR)-4-[6-chloro-5-(difluoromethyl)pyridazin-3-yl]-2,3,4a,5,6,7,8,8a-octahydropyrido[4,3-b][1,4]oxazine (Int 298.5-0.66 mmol, 200 mg), DMAP (0.033 mmol, 4 mg) and triethylamine (0.66 mmol, 90 μl) in 16 ml of DCM was added acetic anhydride (0.98 mmol, 92 μl). The mixture was stirred for 4 h at rt, then diluted with 10 ml of DCM. The organic layer was washed with 10 ml of a saturated aqueous sodium bicarbonate solution and 15 ml of water, then dried over Na₂SO₄, filtered and concentrated under reduced pressure to give the title compound as a yellow oil (222 mg, 98% yield).

LCMS (Method V): Rt=0.78; MS m/z [M+H]⁺=347

### 1-[(4aS,8aR)-4-[5-(difluoromethyl)-6-[2-hydroxy-4-(trifluoromethyl)phenyl]pyridazin-3-yl]-3,4a,5,7,8,8a-hexahydro-2H-pyrido[4,3-b][1,4]oxazin-6-yl]ethanone (Example 386)

Under Ar, to a solution of [2-hydroxy-4-(trifluoromethyl)phenyl]boronic acid (0.58 mmol, 119 mg) and 1-[(4aS,8aR)-4-[6-chloro-5-(difluoromethyl)pyridazin-3-yl]-3,4a,5,7,8,8a-hexahydro-2H-pyrido[4,3-b][1,4]oxazin-6-yl]ethanone (Int 386.1-0.29 mmol, 100 mg) in 6 ml of 1,4-dioxane was added a 2N sodium carbonate aqueous solution (0.78 mmol, 389 μl), then tetrakis(triphenylphosphine)palladium(0) (0.014 mmol, 17 mg). The mixture was stirred at 100° C. for 1 h. After cooling down to rt, the mixture was diluted with 15 ml of EtOAc and 20 ml of diethylether and the organic layer was washed successively with 2×15 ml of a saturated aqueous sodium bicarbonate solution and 10 ml of water. The organic layer was dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 78/10/10/2) to give the title compound as a white solid (53 mg, 39% yield).

Compound Examples 387 and 442 were synthetized analogously to Example 386.

### Example 322: 2-[6-[(4aS,8aR)-2,3,4a,5,6,7,8,8a-octahydropyrido[4,3-b][1,4]oxazin-4-yl]-4-(difluoromethyl)pyridazin-3-yl]-5-methyl-phenol

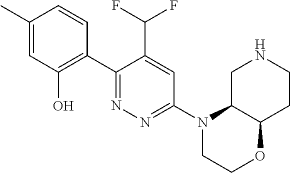 [SMILES: Cc1ccc(-c2nnc(N3CCO[C@@H]4CCNC[C@@H]43)cc2C(F)F)c(O)c1]

### Int 322.1: tert-butyl (4aS,8aR)-4-[5-(difluoromethyl)-6-(2-hydroxy-4-methyl-phenyl)pyridazin-3-yl]-3,4a,5,7,8,8a-hexahydro-2H-pyrido[4,3-b][1,4]oxazine-6-carboxylate

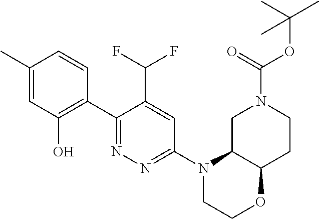 [SMILES: Cc1ccc(-c2nnc(N3CCO[C@@H]4CCN(C(=O)OC(C)(C)C)C[C@@H]43)cc2C(F)F)c(O)c1]

Under Ar, to a solution of 5-methyl-2-(4,4,5,5-tetramethyl-1,3,2-dioxaborolan-2-yl)phenol (0.74 mmol, 173 mg) and tert-butyl (4aS,8aR)-4-[6-chloro-5-(difluoromethyl)pyridazin-3-yl]-3,4a,5,7,8,8a-hexahydro-2H-pyrido[4,3-b][1,4]oxazine-6-carboxylate (Int 298.4-0.49 mmol, 200 mg) in 8 ml of 1,4-dioxane was added a 2N sodium carbonate aqueous solution (1.33 mmol, 667 μl), then tetrakis(triphenylphosphine)palladium(0) (0.025 mmol, 29 mg). The mixture was stirred at 100° C. for 1 h. After cooling down to rt, the mixture was diluted with 20 ml of EtOAc and 20 ml of diethylether and the organic layer was washed successively with 2×15 ml of a saturated aqueous sodium bicarbonate solution and 15 ml of water. The organic layer was dried over Na₂SO₄, filtered and concentrated under reduced pressure. The residue was purified by flash chromatography on silica gel eluting with a gradient of 0 to 40% of EtOAc in cyclohexane to give the title compound (202 mg, 86% yield) as a yellow oil.

LCMS (Method V): Rt=1.19; [M+H]⁺=477

### 2-[6-[(4aS,8aR)-2,3,4a,5,6,7,8,8a-octahydropyrido[4,3-b][1,4]oxazin-4-yl]-4-(difluoromethyl)pyridazin-3-yl]-5-methyl-phenol (Example 322)

Following procedure of Int 268.6, 1-[(4aS,8aR)-4-[6-chloro-5-(difluoromethyl)pyridazin-3-yl]-3,4a,5,7,8,8a-hexahydro-2H-pyrido[4,3-b][1,4]oxazin-6-yl]ethanone (0.38 mmol, 180 mg) gave the title compound as a white solid (101 mg, 51% yield) after purification by flash chromatography on silica gel using a gradient of DCM/MeOH/ACN (from 100/0/0 to 80/10/10).

Compound Example 382 was synthetized analogously to Example 322

### Example 320: 3-methyl-2-[6-[rac-(4aR,7aS)-3,4a,5,6,7,7a-hexahydro-2H-pyrrolo[3,4-b][1,4]oxazin-4-yl]pyridazin-3-yl]-5-(trifluoromethyl)phenol

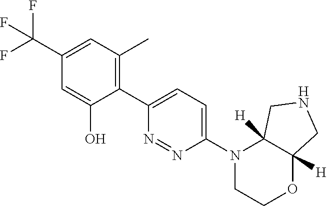 [SMILES: Cc1cc(C(F)(F)F)cc(O)c1-c1ccc(N2CCO[C@H]3CNC[C@H]32)nn1]

### Int 320.1: tert-butyl rac-(4aR,7aS)-4-(6-chloropyridazin-3-yl)-2,3,4a,5,7,7a-hexahydropyrrolo[3,4-b][1,4]oxazine-6-carboxylate

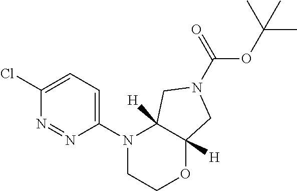 [SMILES: CC(C)(C)OC(=O)N1C[C@@H]2OCCN(c3ccc(Cl)nn3)[C@@H]2C1]

In a sealed tube, a mixture of 3-bromo-6-chloro-pyridazine (1.2 eq., 1.31 g, 0.0068 mol), tert-butyl rac-(4aR,7aS)-3,4,4a,5,7,7a-hexahydro-2H-pyrrolo[3,4-b][1,4]oxazine-6-carboxylate hydrochloride (1.00 g, 0.00378 mol), sodium tert-butoxide (1.09 g, 0.0113 mol), RuPhos Pd G3 (0.158 g, 0.189 mmol) and RuPhos (0.176 g, 0.378 mmol) was purged with N₂. 20 ml of 1,4-dioxane was added, the mixture was purged again with N₂ then heated at 100° C. for 2 h. After cooling down to rt, the mixture was filtered, and the filtrate was concentrated under reduced pressure. The residue was purified by flash chromatography on silica gel using DCM/EtOAc (100/0 to 90/10) to give the title compound as a white solid (1.00 g, 78%).

LCMS (Method S): Rt=1.29; MS m/z [M+H]⁺=285.1

### Int 320.2: tert-butyl rac-(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazine-6-carboxylate

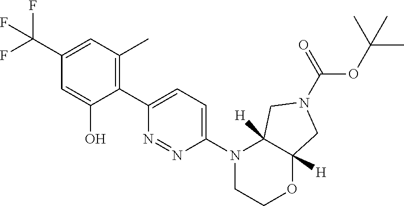 [SMILES: Cc1cc(C(F)(F)F)cc(O)c1-c1ccc(N2CCO[C@H]3CN(C(=O)OC(C)(C)C)C[C@H]32)nn1]

In a sealed tube, a mixture of tert-butyl rac-(4aR,7aS)-4-(6-chloropyridazin-3-yl)-2,3,4a,5,7,7a-hexahydropyrrolo[3,4-b][1,4]oxazine-6-carboxylate (200 mg, 0.587 mmol), K₂CO₃ (243 mg, 1.76 mmol) and 3-methyl-2-(4,4,5,5-tetramethyl-1,3,2-dioxaborolan-2-yl)-5-(trifluoromethyl)phenol (266 mg, 0.880 mmol) in 2 ml of 1,4-dioxane and 0.5 ml of water was purged with N₂. XPhos Pd G2 (23.1 mg, 0.0293 mmol) and XPhos (14.0 mg, 0.0293 mmol) were added, the mixture was purged again with N₂ then heated at 90° C. for 3 h. After cooling down to rt, the mixture was quenched with water and extracted twice with a 80/20 DCM/MeOH mixture. The combined organic layers were dried over MgSO₄, filtered, and concentrated under reduced pressure. The residue was purified by flash chromatography on silica gel using DCM/MeOH (100/0 to 95/5) to give the title compound as a yellow solid (247 mg, 88%).

LCMS (Method S): Rt=1.73; S MS m/z [M+H]+=481.4

### 3-methyl-2-[6-[rac-(4aR,7aS)-3,4a,5,6,7,7a-hexahydro-2H-pyrrolo[3,4-b][1,4]oxazin-4-yl]pyridazin-3-yl]-5-(trifluoromethyl)phenol (Example 320)

At 0° C., to a solution of tert-butyl rac-(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazine-6-carboxylate (247 mg, 0.514 mmol) in 5 ml of 1,4-dioxane was added dropwise 0.65 ml of HCl (4 M in dioxane) and the mixture was stirred at rt overnight. The reaction mixture was concentrated under reduced pressure to give a residue which was triturated in diethyl ether to afford a yellow gum. This gum was taken up in MeOH and toluene and concentrated under reduced pressure, then taken up in MeOH and concentrated under reduced pressure to afford the title compound as a yellow solid (9 mg, 5% yield).

Compound example 318 was synthesized analogously to example 320.

### Examples 325 and 326: (3R)-3-(hydroxymethyl)-1-[6-[2-hydroxy-6-methyl-4-(trifluoromethyl)phenyl]pyridazin-3-yl]pyrrolidin-2-one and (3S)-3-(hydroxymethyl)-1-[6-[2-hydroxy-6-methyl-4-(trifluoromethyl)phenyl]pyridazin-3-yl]pyrrolidin-2-one

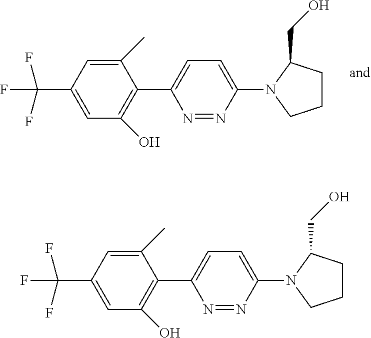 [SMILES: Cc1cc(C(F)(F)F)cc(O)c1-c1ccc(N2CCC[C@@H]2CO)nn1 ; Cc1cc(C(F)(F)F)cc(O)c1-c1ccc(N2CCC[C@H]2CO)nn1]

### Int 325.1: 1-(6-chloropyridazin-3-yl)-3-(hydroxymethyl)pyrrolidin-2-one

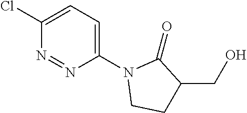 [SMILES: O=C1C(CO)CCN1c1ccc(Cl)nn1]

In a microwave vial, under Ar, a suspension of 3,6-dichloropyridazine (2.14 mmol, 335 mg), 3-(hydroxymethyl)pyrrolidin-2-one (2.56 mmol, 295 mg), potassium phosphate tribasic (4.27 mmol, 907 mg), 1-1′-bis(diphenylphosphino)ferrocene (0.26 mmol, 142 mg) and palladium acetate (0.09 mmol, 19 mg) in 5 ml of 1,4-dioxane was heated at 110° C. under microwave irradiation for 1 h 30. After cooling down to rt, the reaction mixture was diluted with EtOAc and CH₂Cl₂, then filtered. The solid part was washed with EtOAc and CH₂Cl₂ and the filtrate was concentrated under reduced pressure. The residue was purified by flash chromatography on silica gel using DCM/EtOAc (from 100/0 to 60/40) to give the title compound as a brown solid (176 mg, 36% yield).

LCMS (Method B): Rt=0.88; MS m/z [M+H]+=228

### Int 325.2: 3-(hydroxymethyl)-1-[6-[2-hydroxy-6-methyl-4-(trifluoromethyl)phenyl]pyridazin-3-yl]pyrrolidin-2-one

In a microwave vial, under Ar, to a solution of 1-(6-chloropyridazin-3-yl)-3-(hydroxymethyl)pyrrolidin-2-one (0.72 mmol, 164 mg) and 3-methyl-2-(4,4,5,5-tetramethyl-1,3,2-dioxaborolan-2-yl)-5-(trifluoromethyl)phenol (0.76 mmol, 229 mg) in 4 ml of 1,2-dimethoxyethane were added sodium carbonate (1.80 mmol, 191 mg) and 1 ml of water. The solution was purged with Ar, then Pd(PPh₃)₄ (0.04 mmol, 42 mg) was added. The vial was sealed, and the mixture was heated at 100° C. under microwave irradiation for 1 h.

After cooling down to rt, the reaction mixture was diluted with EtOAc and water, then filtered. The organic layer was separated, dried over Na₂SO₄, filtered and concentrated under reduced pressure. The residue was purified by flash chromatography on silica gel using DCM/MeOH/ACN (100/0/0 to 90/5/5) to give the title compound as a beige solid (148 mg, 56% yield).

LCMS (Method B): Rt=1.36; MS m/z [M+H]+=368

### (3R)-3-(hydroxymethyl)-1-[6-[2-hydroxy-6-methyl-4-(trifluoromethyl)phenyl]pyridazine-3-yl]pyrrolidin-2-one and (3S)-3-(hydroxymethyl)-1-[6-[2-hydroxy-6-methyl-4-(trifluoromethyl)phenyl]pyridazin-3-yl]pyrrolidin-2-one (Example 325 and Example 326)

The 2 enantiomers of 3-(hydroxymethyl)-1-[6-[2-hydroxy-6-methyl-4-(trifluoromethyl)phenyl]pyridazin-3-yl]pyrrolidin-2-one (146 mg) were separated by chiral phase LC (Column Chiralcel OJ-H 5 μm, 350×76.5 mm, Flow rate 400 ml/min, eluent (n-heptane/ethanol 80/20)+0.1% TEA, to give both enantiomers, first eluting Rt=13.1 min, Example 325 (43.4 mg, yield 29%) and second eluting Rt=17.7 min, Example 326 (69.4 mg, yield 47%). The absolute configurations cannot be assigned at this stage.

### Examples 333 and 334: 2-[6-[(4aR,7aS)-6-methyl-2,3,4a,5,7,7a-hexahydropyrrolo[3,4-b][1,4]oxazin-4-yl]pyridazin-3-yl]-3-methyl-5-(trifluoromethyl)phenol and 2-[6-[(4aS,7aR)-6-methyl-2,3,4a,5,7,7a-hexahydropyrrolo[3,4-b][1,4]oxazin-4-yl]pyridazin-3-yl]-3-methyl-5-(trifluoromethyl)phenol

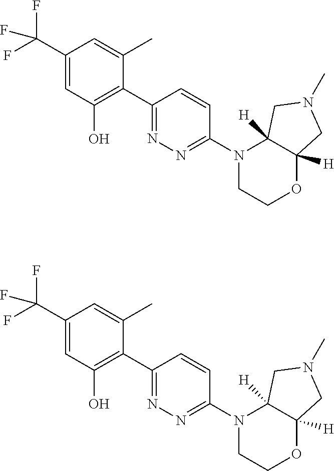 [SMILES: Cc1cc(C(F)(F)F)cc(O)c1-c1ccc(N2CCO[C@H]3CN(C)C[C@H]32)nn1 ; Cc1cc(C(F)(F)F)cc(O)c1-c1ccc(N2CCO[C@@H]3CN(C)C[C@@H]32)nn1]

### Int 333.1: tert-butyl rac-(4aR,7aS)-4-(6-chloropyridazin-3-yl)-2,3,4a,5,7,7a-hexahydropyrrolo[3,4-b][1,4]oxazine-6-carboxylate

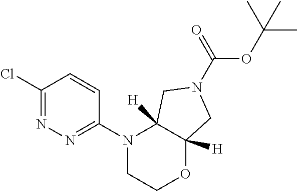 [SMILES: CC(C)(C)OC(=O)N1C[C@@H]2OCCN(c3ccc(Cl)nn3)[C@@H]2C1]

In a sealed tube, a mixture of 3-bromo-6-chloro-pyridazine (1.31 g, 6.8 mmol), tert-butyl rac-(4aR,7aS)-3,4,4a,5,7,7a-hexahydro-2H-pyrrolo[3,4-b][1,4]oxazine-6-carboxylate hydrochloride (1.00 g, 3.78 mmol), sodium tert-butoxide (1.09 g, 11.3 mmol), RuPhos Pd G3 (0.158 g, 0.189 mmol) and RuPhos (0.176 g, 0.378 mmol) was purged with N₂. 20 ml of 1,4-dioxane was added, the mixture was purged again with N₂ then heated at 100° C. for 2 h. After cooling down to rt, the mixture was filtered, and the filtrate was concentrated under reduced pressure. The residue was purified by flash chromatography on silica gel using DCM/EtOAc (100/0 to 90/10) to give the title compound as a white solid (1.00 g, 78%).

LCMS (Method S): Rt=1.29 min; MS m/z [M+H]⁺=285.1

### Int 333.2: rac-(4aR,7aS)-4-(6-chloropyridazin-3-yl)-3,4a,5,6,7,7a-hexahydro-2H-pyrrolo[3,4-b][1,4]oxazine;2,2,2-trifluoroacetic acid

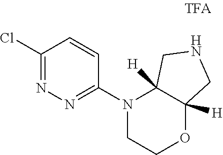 [SMILES: Clc1ccc(N2CCO[C@H]3CNC[C@H]32)nn1]

To a solution of tert-butyl rac-(4aR,7aS)-4-(6-chloropyridazin-3-yl)-2,3,4a,5,7,7a-hexahydropyrrolo[3,4-b][1,4]oxazine-6-carboxylate (100 mg, 0.293 mmol) in 3 ml of dichloromethane was slowly added TFA (0.218 mL, 2.93 mmol). The solution was stirred at rt for 18 h. The reaction mixture was concentrated under reduced pressure to give the title compound as a TFA salt (194 mg), which was used without further purification in the next step.

1H NMR (500 MHz, DMSO-d6) δ (ppm): 9.00 (s, 1H), 7.66 (d, J=9.6 Hz, 1H), 7.41 (d, J=9.6 Hz, 1H), 5.01 (td, J=9.2, 3.6 Hz, 1H), 4.21 (t, J=3.6 Hz, 1H), 4.04 (dd, J=11.8, 3.5 Hz, 1H), 3.84 (dd, J=13.1, 2.8 Hz, 1H), 3.67 (td, J=12.0, 2.9 Hz, 1H), 3.53-3.40 (m, 2H), 3.36-3.28 (m, 1H), 3.24-3.15 (m, 1H), 3.14-3.07 (m, 1H).

### Int 333.3: rac-(4aR,7aS)-4-(6-chloropyridazin-3-yl)-6-methyl-2,3,4a,5,7,7a-hexahydropyrrolo[3,4-b][1,4]oxazine

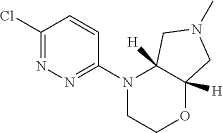 [SMILES: CN1C[C@@H]2OCCN(c3ccc(Cl)nn3)[C@@H]2C1]

formaldehyde (37% in water) (0.262 mL, 0.00332 mol) was added to a solution of rac-(4aR,7aS)-4-(6-chloropyridazin-3-yl)-3,4a,5,6,7,7a-hexahydro-2H-pyrrolo[3,4-b][1,4]oxazine;2,2,2-trifluoroacetic acid (0.160 g, 0.000665 mol) in 0.8 ml of methanol and 0.8 ml of tetrahydrofuran. The solution was stirred for 15 min, then NaBH₃CN (0.0418 g, 0.000665 mol) was added. The solution was stirred at rt for 1 h. Then, EtOAc was added to the mixture. The mixture was then quenched with a small amount of water and basified with a 1N aqueous solution of NaOH until pH 8. The aqueous layer was separated then extracted twice with EtOAc. The combined organic layers were washed with a saturated aqueous solution of NaCl, dried over MgSO₄, filtered and concentrated under reduced pressure. The residue was purified by flash chromatography on silica gel using DCM/MeOH (100/0 to 90/10) to give the title compound (0.0740 g, 44%).

LCMS (Method S): Rt=0.75 min; MS m/z [M+H]+=255.2

### Int 333.4: 3-methyl-2-[6-[rac-(4aR,7aS)-6-methyl-2,3,4a,5,7,7a-hexahydropyrrolo[3,4-b][1,4]oxazin-4-yl]pyridazin-3-yl]-5-(trifluoromethyl)phenol

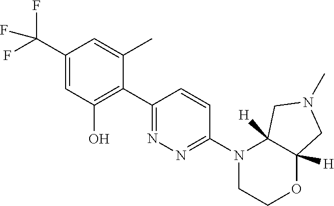 [SMILES: Cc1cc(C(F)(F)F)cc(O)c1-c1ccc(N2CCO[C@H]3CN(C)C[C@H]32)nn1]

In a sealed tube, a mixture of rac-(4aR,7aS)-4-(6-chloropyridazin-3-yl)-6-methyl-2,3,4a,5,7,7a-hexahydropyrrolo[3,4-b][1,4]oxazine (0.0720 g, 0.000283 mol), K₂CO₃ (0.0781 g, 0.000565 mol) and 3-methyl-2-(4,4,5,5-tetramethyl-1,3,2-dioxaborolan-2-yl)-5-(trifluoromethyl)phenol (0.128 g, 0.000424 mol) in 1.6 ml of 1,4-dioxane and 0.4 ml of water was purged with N₂. XPhos Pd G2 (0.0111 g, 1.41e-5 mol) and XPhos (0.00674 g, 1.41e-5 mol) were added, the mixture was purged again with N₂ then heated at 90° C. for 3 h. After cooling down to rt, EtOAc and water were added. The aqueous layer was separated and extracted twice with EtOAc. The combined organic layers were washed with a saturated aqueous solution of NaCl then dried over MgSO₄, filtered and concentrated under reduced pressure. The residue was purified by flash chromatography on silica gel using DCM/(MeOH/aq NH3 (9:1)) (100/0 to 0/100) to give the title compound as an off-white solid (0.0780 g, 70%).

LCMS (Method E): Rt=2.49 min; MS m/z [M+H]+=395.4

### 2-[6-[(4aR,7aS)-6-methyl-2,3,4a,5,7,7a-hexahydropyrrolo[3,4-b][1,4]oxazin-4-yl]pyridazin-3-yl]-3-methyl-5-(trifluoromethyl)phenol and 2-[6-[(4aS,7aR)-6-methyl-2,3,4a,5,7,7a-hexahydropyrrolo[3,4-b][1,4]oxazin-4-yl]pyridazin-3-yl]-3-methyl-5-(trifluoromethyl)phenol (Example 333 and Example 334)

3-methyl-2-[6-[rac-(4aR,7aS)-6-methyl-2,3,4a,5,7,7a-hexahydropyrrolo[3,4-b][1,4]oxazin-4-yl]pyridazin-3-yl]-5-(trifluoromethyl)phenol was purified by chiral SFC (Stationary phase: Chiralpak® AD-H 20×250 mm, Mobile phase: CO₂/(EtOH+0.3% iPrNH₂) 80/20). The fractions containing compound were combined and evaporated in vacuo to give 2 fractions. The fractions containing each compound were combined and concentrated under reduced pressure to give two residues. The first residue was lyophilized to provide 2-[6-[(4aR,7aS)-6-methyl-2,3,4a,5,7,7a-hexahydropyrrolo[3,4-b][1,4]oxazin-4-yl]pyridazin-3-yl]-3-methyl-5-(trifluoromethyl)phenol (20 mg, 18%). The second residue was lyophilized to provide 2-[6-[(4aS,7aR)-6-methyl-2,3,4a,5,7,7a-hexahydropyrrolo[3,4-b][1,4]oxazin-4-yl]pyridazin-3-yl]-3-methyl-5-(trifluoromethyl)phenol (23 mg, 21%). The absolute configurations cannot be assigned at this stage.

Compounds Example 289, 361 and 376 were synthesized analogously to compound Example 334 without chiral separation.

### Example 335: 1-[rac-(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone

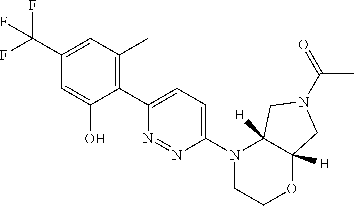 [SMILES: CC(=O)N1C[C@@H]2OCCN(c3ccc(-c4c(C)cc(C(F)(F)F)cc4O)nn3)[C@@H]2C1]

### Int 335.1: tert-butyl rac-(4aR,7aS)-4-(6-chloropyridazin-3-yl)-2,3,4a,5,7,7a-hexahydropyrrolo[3,4-b][1,4]oxazine-6-carboxylate

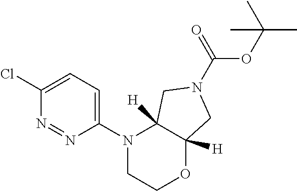 [SMILES: CC(C)(C)OC(=O)N1C[C@@H]2OCCN(c3ccc(Cl)nn3)[C@@H]2C1]

In a sealed tube, a mixture of 3-bromo-6-chloro-pyridazine (1.31 g, 6.8 mmol), tert-butyl rac-(4aR,7aS)-3,4,4a,5,7,7a-hexahydro-2H-pyrrolo[3,4-b][1,4]oxazine-6-carboxylate hydrochloride (1.00 g, 3.78 mmol), sodium tert-butoxide (1.09 g, 11.3 mmol), RuPhos Pd G3 (0.158 g, 0.189 mmol) and RuPhos (0.176 g, 0.378 mmol) was purged with N₂. 20 ml of 1,4-dioxane was added, the mixture was purged again with N₂ then heated at 100° C. for 2 h. After cooling down to rt, the mixture was filtered, and the filtrate was concentrated under reduced pressure. The residue was purified by flash chromatography on silica gel using DCM/EtOAc (100/0 to 90/10) to give the title compound as a white solid (1.00 g, 78%).

LCMS (Method S): Rt=1.29; MS m/z [M+H]+=285.1

### Int 335.2: rac-(4aR,7aS)-4-(6-chloropyridazin-3-yl)-3,4a,5,6,7,7a-hexahydro-2H-pyrrolo[3,4-b][1,4]oxazine

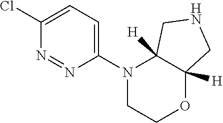 [SMILES: Clc1ccc(N2CCO[C@H]3CNC[C@H]32)nn1]

To a solution of tert-butyl rac-(4aR,7aS)-4-(6-chloropyridazin-3-yl)-2,3,4a,5,7,7a-hexahydropyrrolo[3,4-b][1,4]oxazine-6-carboxylate (100 mg, 0.293 mmol) in 3 ml of dichloromethane at rt was slowly added TFA (0.218 mL, 2.93 mmol) and the solution was stirred at room temperature for 18 h. The reaction mixture was concentrated under reduced pressure to give the title compound (194 mg) which was used without further purification. 1H NMR (500 MHz, DMSO-d6) δ (ppm): 9.00 (s, 1H), 7.66 (d, J=9.6 Hz, 1H), 7.41 (d, J=9.6 Hz, 1H), 5.01 (td, J=9.2, 3.6 Hz, 1H), 4.21 (t, J=3.6 Hz, 1H), 4.04 (dd, J=11.8, 3.5 Hz, 1H), 3.84 (dd, J=13.1, 2.8 Hz, 1H), 3.67 (td, J=12.0, 2.9 Hz, 1H), 3.53-3.40 (m, 2H), 3.36-3.28 (m, 1H), 3.24-3.15 (m, 1H), 3.14-3.07 (m, 1H).

### Int 335.3: 1-[(4aR,7aS)-4-(6-chloropyridazin-3-yl)-2,3,4a,5,7,7a-hexahydropyrrolo[3,4-b][1,4]oxazin-6-yl]ethanone

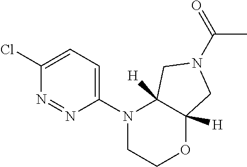 [SMILES: CC(=O)N1C[C@@H]2OCCN(c3ccc(Cl)nn3)[C@@H]2C1]

At 0° C., acetyl chloride (0.0650 mL, 0.914 mmol) was added dropwise to a solution of rac-(4aR,7aS)-4-(6-chloropyridazin-3-yl)-3,4a,5,6,7,7a-hexahydro-2H-pyrrolo[3,4-b][1,4]oxazine (200 mg, 0.831 mmol) and DIPEA (0.217 mL, 1.25 mmol) in 4 ml of DCM and the mixture was stirred for 1 h at rt. The mixture was then quenched with water and extracted with DCM. The organic layer was dried over MgSO₄, filtered and concentrated under reduced pressure to give the title compound (231 mg, yield: 98%).

LCMS (Method R): Rt=0.89; MS m/z [M+H]+=283.2

### 1-[rac-(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone (Example 335)

In a microwave vial, under N₂, a mixture of 1-[rac-(4aR,7aS)-4-(6-chloropyridazin-3-yl)-2,3,4a,5,7,7a-hexahydropyrrolo[3,4-b][1,4]oxazin-6-yl]ethanone (230 mg, 0.814 mmol), K₂CO₃ (225 mg, 1.63 mmol), 3-methyl-2-(4,4,5,5-tetramethyl-1,3,2-dioxaborolan-2-yl)-5-(trifluoromethyl)phenol (369 mg, 01.22 mmol) in 4 ml of 1,4-dioxane and 1 ml of water was degazed under N₂ flow. XPhos (19.4 mg, 0.0407 mmol) and XPhos Pd G2 (32.0 mg, 0.0407 mmol) were added, and the mixture was heated at 90° C. for 3 h. After cooling down to rt, the reaction mixture was concentrated under reduced pressure. The residue was purified by flash chromatography on silica gel using DCM/MeOH (100/0 to 95/5) to give a yellow solid residue which was again purified by prep-HPLC (Column YMC—Actus Triart Prep C18-S 150*30 mm 5 μm Flow rate 50 ml/min-Focused gradient MeCN/aq·NH₄HCO₃ 0.2% pH=7.9 Focused gradient from 35/65 to 55/45. The pure fractions were diluted with dichloromethane and washed with a saturated aqueous sodium bicarbonate solution. The organic layer was dried over Na₂SO₄, filtered and concentrated under reduced pressure to give the title compound as a white solid (170 mg, 47% yield).

Compound Example 337 was synthesized analogously to Example 335

### Examples 348 and 352: 1-[(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone and 1-[(4aS,7aR)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone

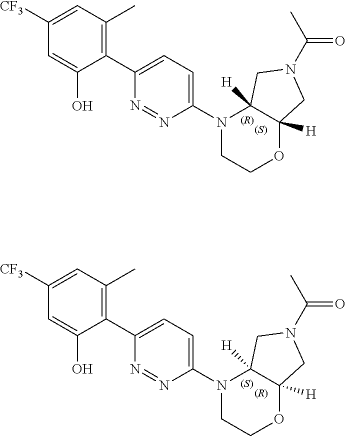

1-[rac-(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone (example 335-160 mg) was purified by preparative chiral SFC (Stationary phase: Chiralpak® AD-H 20×250 mm, Mobile phase: CO₂/iPrOH 70/30. The fractions containing each compound were combined and concentrated under reduced pressure to give two residues. The first residue was lyophilized to provide 1-[(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone (28 mg, 8%). The second residue (40 mg, 11%) was purified by reverse phase preparative LC (Column YMC—Actus Triart Prep C18-S 150*30 mm 5 μm Flow rate 50 ml/min-Focused gradient MeCN/aq·NH₄HCO₃ 0.2% pH=7.9 Focused gradient from 25/75 to 50/50 15CV) to give after concentration under reduced pressure 1-[(4aS,7aR)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone (13 mg, 4%). The absolute configurations cannot be assigned at this stage.

### Example 321: 2-[6-[(4aS,8aR)-6-methyl-3,4a,5,7,8,8a-hexahydro-2H-pyrido[4,3-b][1,4]oxazin-4-yl]pyridazin-3-yl]-5-chloro-3-methyl-phenol

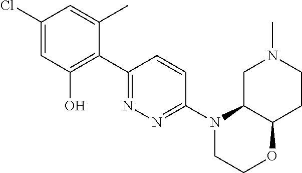 [SMILES: Cc1cc(Cl)cc(O)c1-c1ccc(N2CCO[C@@H]3CCN(C)C[C@@H]32)nn1]

### Int 321.1: tert-butyl (4aS,8aR)-4-(6-chloropyridazin-3-yl)-3,4a,5,7,8,8a-hexahydro-2H-pyrido[4,3-b][1,4]oxazine-6-carboxylate

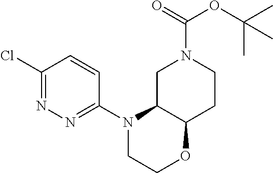 [SMILES: CC(C)(C)OC(=O)N1CC[C@H]2OCCN(c3ccc(Cl)nn3)[C@H]2C1]

A solution of tert-butyl (4aS,8aR)-2,3,4,4a,5,7,8,8a-octahydropyrido[4,3-b][1,4]oxazine-6-carboxylate (Int 298.3-24.76 mmol, 6 g), 3,6-dichloropyridazine (37.14 mmol, 5.53 g) and triethylamine (74.28 mmol, 10.4 ml) in 20 ml of 1-butanol was stirred at 150° C. for 1 h while removing 1-butanol and triethylamine under a stream of argon. The mixture was stirred at 150° C. for another 1 h 30 then cooled down to rt. The mixture was triturated with 200 ml of EtOAc and filtered. The cake was rinsed with 2×50 ml of EtOAc and the filtrates were concentrated under reduced pressure. The residue was purified by flash chromatography on silica gel eluting with a gradient of 0 to 100% of EtOAc in cyclohexane to give the title compound (3.57 g, 41% yield) as a yellow oil.

LCMS (Method V): Rt=1.07; MS m/z [M+H]⁺=355

### Int 321.2: (4aS,8aR)-4-(6-chloropyridazin-3-yl)-2,3,4a,5,6,7,8,8a-octahydropyrido[4,3-b][1,4]oxazine

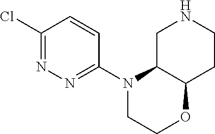 [SMILES: Clc1ccc(N2CCO[C@@H]3CCNC[C@@H]32)nn1]

A solution of tert-butyl (4aS,8aR)-4-(6-chloropyridazin-3-yl)-3,4a,5,7,8,8a-hexahydro-2H-pyrido[4,3-b][1,4]oxazine-6-carboxylate (7.05 mmol, 2.5 g) in 45 ml of dichloromethane and 5 ml of trifluoroacetic acid was stirred at rt for 18 h, then concentrated under reduced pressure. The residue was diluted with 50 ml of dichloromethane and washed with 50 ml of a saturated aqueous sodium bicarbonate solution and 10 ml of a 2N aqueous sodium carbonate solution. The pooled aqueous layers were extracted with 4×30 ml of DCM and the combined organic layers were dried over Na₂SO₄, filtered and concentrated under reduced pressure to give the title compound (1.79 g, 100% yield) as a colorless oil.

LCMS (Method W): Rt=0.58; MS m/z [M+H]⁺=255

### Int 321.3: (4aS,8aR)-4-(6-chloropyridazin-3-yl)-6-methyl-3,4a,5,7,8,8a-hexahydro-2H-pyrido[4,3-b][1,4]oxazine

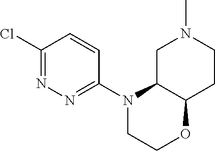 [SMILES: CN1CC[C@H]2OCCN(c3ccc(Cl)nn3)[C@H]2C1]

Under Ar, to a solution of (4aS,8aR)-4-(6-chloropyridazin-3-yl)-2,3,4a,5,6,7,8,8a-octahydropyrido[4,3-b][1,4]oxazine (7.05 mmol, 1.79 g) cooled to 0-5° C. were successively added 7 ml of acetic acid, a 37% aqueous solution of formaldehyde (35 mmol, 2.7 ml) and sodium triacetoxyborohydride (13 mmol, 2.7 g) portion wise. The mixture was stirred at rt for 1 h30). 100 ml of dichloromethane, 100 ml of a saturated aqueous sodium bicarbonate solution and 20 ml of a 2N aqueous sodium carbonate solution were added. The aqueous layer was extracted with 100 ml of dichloromethane and the pooled organic layers were dried over Na₂SO₄, filtered, and concentrated under reduced pressure to give the title compound (1.86 g, 98% yield) as a yellow oil.

LCMS (Method W): Rt=0.60; MS m/z [M+H]⁺=269

### 2-[6-[(4aS,8aR)-6-methyl-3,4a,5,7,8,8a-hexahydro-2H-pyrido[4,3-b][1,4]oxazin-4-yl]pyridazin-3-yl]-5-chloro-3-methyl-phenol (Example 321)

Under Ar, to a solution of 5-chloro-3-methyl-2-(4,4,5,5-tetramethyl-1,3,2-dioxaborolan-2-yl)phenol (2.54 mmol, 683 mg) and (4aS,8aR)-4-(6-chloropyridazin-3-yl)-6-methyl-3,4a,5,7,8,8a-hexahydro-2H-pyrido[4,3-b][1,4]oxazine (Int 321.3-1.41 mmol, 380 mg) in 20 ml of 1,4-dioxane was successively added a 2N sodium carbonate aqueous solution (3.82 mmol, 1.91 ml) and tetrakis(triphenylphosphine)palladium(0) (0.071 mmol, 82 mg). The mixture was stirred at 100° C. for 2 h. After cooling down to rt, the mixture was diluted with 100 ml of EtOAc and 100 ml of diethylether. The organic layer was washed successively with 2×100 ml of a saturated aqueous sodium bicarbonate solution and 100 ml of water. The organic layer was dried over Na₂SO₄, filtered and concentrated under reduced pressure. The residue was purified by flash chromatography on silica gel using a gradient of DCM/MeOH/ACN (from 100/0/0 to 90/5/5) to give, after trituration in diethylether, the title compound as a white solid (207 mg, 39% yield).

### Example 311: 2-[6-[(4aS,8aR)-6-methyl-3,4a,5,7,8,8a-hexahydro-2H-pyrido[4,3-b][1,4]oxazin-4-yl]pyridazin-3-yl]-3,5-dimethyl-phenol

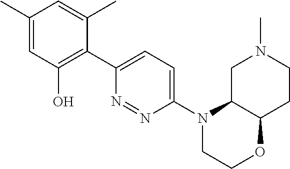 [SMILES: Cc1cc(C)c(-c2ccc(N3CCO[C@@H]4CCN(C)C[C@@H]43)nn2)c(O)c1]

Under Ar, to a solution of (2-hydroxy-4,6-dimethyl-phenyl)boronic acid (2.23 mmol, 371 mg) and (4aS,8aR)-4-(6-chloropyridazin-3-yl)-6-methyl-3,4a,5,7,8,8a-hexahydro-2H-pyrido[4,3-b][1,4]oxazine (Int 321.3-1.49 mmol, 400 mg) in 20 ml of 1,4-dioxane was added a 2N sodium carbonate aqueous solution (4.02 mmol, 2 ml) then tetrakis(triphenylphosphine)palladium(0) (0.074 mmol, 86 mg). The mixture was stirred at 100° C. for 1 h. After cooling down to rt, the mixture was diluted with 100 ml of EtOAc and 100 ml of diethylether and the organic layer was washed successively with 2×100 ml of a saturated aqueous sodium bicarbonate solution and 100 ml of water. The organic layer was dried over Na₂SO₄, filtered and concentrated under reduced pressure. The residue was purified by flash chromatography on silica gel using a gradient of DCM/MeOH/ACN (from 100/0/0 to 85/7.5/7.5) to give a solid that was triturated in the minimum of acetonitrile to give the title compound as a white solid (214 mg, 40% yield).

### Example 313: 2-[6-[(4aS,8aR)-6-ethyl-3,4a,5,7,8,8a-hexahydro-2H-pyrido[4,3-b][1,4]oxazin-4-yl]pyridazin-3-yl]-5-chloro-3-methyl-phenol

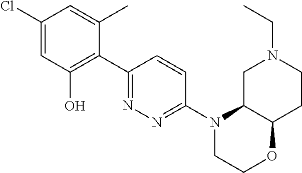 [SMILES: CCN1CC[C@H]2OCCN(c3ccc(-c4c(C)cc(Cl)cc4O)nn3)[C@H]2C1]

### Int 313.1: (4aS,8aR)-4-(6-chloropyridazin-3-yl)-6-ethyl-3,4a,5,7,8,8a-hexahydro-2H-pyrido[4,3-b][1,4]oxazine

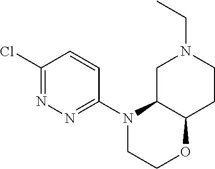 [SMILES: CCN1CC[C@H]2OCCN(c3ccc(Cl)nn3)[C@H]2C1]

To a solution of (4aS,8aR)-4-(6-chloropyridazin-3-yl)-2,3,4a,5,6,7,8,8a-octahydropyrido[4,3-b][1,4]oxazine (Int 321.2-2.81 mmol, 720 mg) in 10 ml of dichloromethane were added successively 2.4 ml of acetic acid, acetaldehyde (14 mmol, 787 μl) and sodium triacetoxyborohydride (5.07 mmol, 1.07 g) portionwise. The mixture was stirred at rt for 18 h. The mixture was diluted with 100 ml of dichloromethane and 100 ml of a saturated aqueous sodium bicarbonate solution. The aqueous layer was extracted with 100 ml of DCM then with 2×100 ml of a mixture DCM/isopropanol: 90/10. The combined organic layers were dried over Na₂SO₄, filtered and concentrated under reduced pressure. The residue was purified by flash chromatography on silica gel using a gradient of DCM/MeOH/ACN (from 100/0/0/0 to 80/10/10) to give the title compound as a white solid (795 mg, 99% yield).

LCMS (Method V): Rt=0.64; MS m/z [M+H]⁺=283

### 2-[6-[(4aS,8aR)-6-ethyl-3,4a,5,7,8,8a-hexahydro-2H-pyrido[4,3-b][1,4]oxazin-4-yl]pyridazin-3-yl]-5-chloro-3-methyl-phenol (Example 313)

Under Ar, to a solution of (4-chloro-2-hydroxy-6-methyl-phenyl)boronic acid (2.48 mmol, 463 mg) and (4aS,8aR)-4-(6-chloropyridazin-3-yl)-6-ethyl-3,4a,5,7,8,8a-hexahydro-2H-pyrido[4,3-b][1,4]oxazine (Int 313.1-1.38 mmol, 390 mg) in 20 ml of 1,4-dioxane were successively added a 2N sodium carbonate aqueous solution (3.72 mmol, 1.86 ml) and tetrakis(triphenylphosphine)palladium(0) (0.069 mmol, 80 mg). The mixture was stirred at 100° C. for 2 h. After cooling down to rt, the mixture was diluted with 100 ml of EtOAc and 100 ml of diethylether and the organic layer was washed successively with 2×100 ml of a saturated aqueous sodium bicarbonate solution and 100 ml of water. The organic layer was dried over Na₂SO₄, filtered and concentrated under reduced pressure. The residue was purified by flash chromatography on silica gel using a gradient of DCM/MeOH/ACN (from 100/0/0/0 to 95/2.5/2.5) to give the title compound as a white solid (240 mg, 45% yield).

### Example 377: 1-[rac-(3aS,6aS)-1-[6-[2-hydroxy-6-methyl-4-(trifluoromethyl)phenyl]pyridazin-3-yl]-2,3,3a,5,6,6a-hexahydropyrrolo[3,2-b]pyrrol-4-yl]ethanone

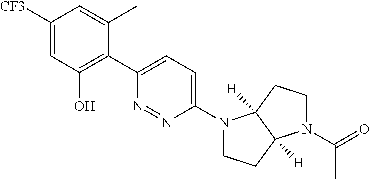 [SMILES: CC(=O)N1CC[C@H]2[C@@H]1CCN2c1ccc(-c2c(C)cc(C(F)(F)F)cc2O)nn1]

### Int 377.1: tert-butyl rac-(3aS,6aS)-1-(6-chloropyridazin-3-yl)-2,3,3a,5,6,6a-hexahydropyrrolo[3,2-b]pyrrole-4-carboxylate

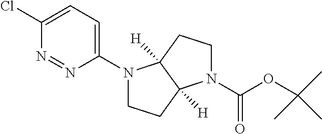 [SMILES: CC(C)(C)OC(=O)N1CC[C@H]2[C@@H]1CCN2c1ccc(Cl)nn1]

To a solution of 3,6-dichloropyridazine (400 mg, 2.685 mmol) in 7 ml of 1-Butanol was added tert-butyl rac-(3aS,6aS)-2,3,3a,5,6,6a-hexahydro-1H-pyrrolo[3,2-b]pyrrole-4-carboxylate (627 mg, 2.9535 mmol) and Triethylamine (0.823 ml, 5.9069 mmol). The mixture was heated at 130° C. for 5 h. After cooling down to rt, the mixture was diluted with AcOEt, washed with water and brine, dried over MgSO₄, concentrated under reduced pressure. The residue was purified by flash chromatography on silica gel eluting with a gradient of Hept/AcOEt (from 100/0 to 50/50) to give the title compound as a white solid (575 mg, 66% yield).

LCMS (Method L): Rt=0.88 min; MS m/z [M+H]+=325

### Int 377.2: rac-(3aS,6aS)-4-(6-chloropyridazin-3-yl)-2,3,3a,5,6,6a-hexahydro-1H-pyrrolo [3,2-b]pyrrole; dihydrochloride

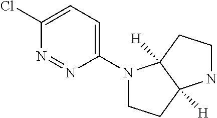 [SMILES: Clc1ccc(N2CC[C@@H]3NCC[C@@H]32)nn1]

To a solution of tert-butyl rac-(3aS,6aS)-1-(6-chloropyridazin-3-yl)-2,3,3a,5,6,6a-hexahydropyrrolo[3,2-b]pyrrole-4-carboxylate (575 mg, 1.7703 mmol) in 10 ml of DCM was added a solution of HCl 4N dioxane (8.85 ml, 35.4058 mmol). After stirring 4 h at rt, the mixture was concentrated under reduce pressure to give the title compound as a beige solid (m=600 mg), which was used without further purification in the next step.

### Int 377.3: 1-[rac-(3aS,6aS)-1-(6-chloropyridazin-3-yl)-2,3,3a,5,6,6a-hexahydropyrrolo [3,2-b]pyrrol-4-yl]ethanone

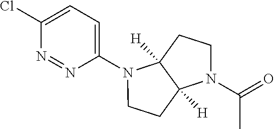 [SMILES: CC(=O)N1CC[C@H]2[C@@H]1CCN2c1ccc(Cl)nn1]

To a suspension of rac-(3aS,6aS)-4-(6-chloropyridazin-3-yl)-2,3,3a,5,6,6a-hexahydro-1H-pyrrolo[3,2-b]pyrrole; dihydrochloride (200 mg, 0.672 mmol) in 40 ml of DCM was added N,N-dimethylpyridin-4-amine (4 mg, 0.0336 mol) and triethylamine (0.28 ml, 2.016 mmol). Acetic anhydride (0.10 ml, 1.01 mmol) was added and the mixture was stirred at rt for 4 h. The mixture was diluted with water, extracted with DCM. The organic layer was washed with an aqueous saturated solution of NaHCO₃, brine, dried over Mg₂SO₄, filtered, and concentrated under reduce pressure. The residue was purified by flash chromatography on silica gel eluting with a gradient of DCM/MeOH (100/0 to 95/5) to give the title compound as a solid (101 mg, 56% yield).

LCMS (Method L): Rt=0.46 min; MS m/z [M+H]+=267

### 1-[rac-(3aS,6aS)-1-[6-[2-hydroxy-6-methyl-4-(trifluoromethyl)phenyl]pyridazin-3-yl]-2,3,3a,5,6,6a-hexahydropyrrolo[3,2-b]pyrrol-4-yl]ethanone (Example 377)

In a microwave vial, under Ar, to a solution of 1-[rac-(3aS,6aS)-1-(6-chloropyridazin-3-yl)-2,3,3a,5,6,6a-hexahydropyrrolo[3,2-b]pyrrol-4-yl]ethanone (145 mg, 0.5436 mmol), 3-methyl-2-(4,4,5,5-tetramethyl-1,3,2-dioxaborolan-2-yl)-5-(trifluoromethyl)phenol (180 mg, 0.598 mmol) and Sodium carbonate (144 mg, 1.3591 mmol) in 4 ml of 1,2-Dimethoxyethane and 1 ml of water was added tetrakis(triphenylphosphine)Palladium(0) (31 mg, 0.0272 mmol). The mixture was heated at 100° C. under microwave irradiation for 1 h30. After cooling down to rt, the reaction mixture was diluted with AcOEt, washed with water and brine, dried over MgSO₄, concentrated under reduced pressure. The residue was purified by flash chromatography on silica gel eluting with a gradient of DCM/MeOH (100/0 to 95/5) to give, after crystallization in diethylether, the title compound as a white solid (103 mg, 47% yield).

### Example 404: 2-[6-[(4aS,8aR)-6-ethyl-3,4a,5,7,8,8a-hexahydro-2H-pyrido[4,3-b][1,4]oxazin-4-yl]pyridazin-3-yl]-3,5-dimethyl-phenol

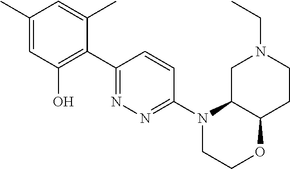 [SMILES: CCN1CC[C@H]2OCCN(c3ccc(-c4c(C)cc(C)cc4O)nn3)[C@H]2C1]

Under Ar, to a solution of (2-hydroxy-4,6-dimethyl-phenyl)boronic acid (2.55 mmol, 423 mg) and (4aS,8aR)-4-(6-chloropyridazin-3-yl)-6-ethyl-3,4a,5,7,8,8a-hexahydro-2H-pyrido[4,3-b][1,4]oxazine (Int 313.1-1.42 mmol, 402 mg) in 20 ml of 1,4-dioxane were successively added a 2N sodium carbonate aqueous solution (3.82 mmol, 1.91 ml) and tetrakis(triphenylphosphine)palladium(0) (0.071 mmol, 82 mg). The mixture was stirred at 100° C. for 2 h. After cooling down to rt, the mixture was diluted with 100 ml of EtOAc and 100 ml of diethylether and the organic layer was washed successively with 2×100 ml of a saturated aqueous sodium bicarbonate solution and 100 ml of water. The organic layer was dried over Na₂SO₄, filtered and concentrated under reduced pressure. The residue was purified by flash chromatography on silica gel using a gradient of DCM/MeOH/ACN (from 100/0/0/0 to 95/2.5/2.5) to give the title compound as a white solid (201 mg, 39% yield).

### Example 408: 2-[6-[(4aS,8aR)-6-ethyl-3,4a,5,7,8,8a-hexahydro-2H-pyrido[4,3-b][1,4]oxazin-4-yl]pyridazin-3-yl]-3-ethyl-phenol

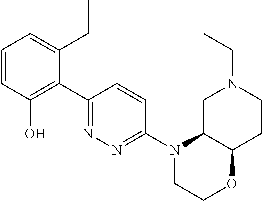 [SMILES: CCc1cccc(O)c1-c1ccc(N2CCO[C@@H]3CCN(CC)C[C@@H]32)nn1]

Under Ar, to a solution of (2-ethyl-6-hydroxy-phenyl)boronic acid (Int 358.2-1.01 mmol, 167 mg) and (4aS,8aR)-4-(6-chloropyridazin-3-yl)-6-ethyl-3,4a,5,7,8,8a-hexahydro-2H-pyrido[4,3-b][1,4]oxazine (Int 313.1-0.67 mmol, 190 mg) in 20 ml of 1,4-dioxane were successively added a 2N sodium carbonate aqueous solution (1.81 mmol, 0.91 ml) and tetrakis(triphenylphosphine)palladium(0) (0.034 mmol, 39 mg). The mixture was stirred at 100° C. for 2 h. After cooling down to rt, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered and concentrated under reduced pressure. The residue was purified by flash chromatography on silica gel using a gradient of DCM/MeOH/ACN (from 100/0/0/0 to 95/2.5/2.5) to give the title compound as a white solid (68 mg, 27% yield).

### Example 412: 2-[6-[(4aS,8aR)-6-ethyl-3,4a,5,7,8,8a-hexahydro-2H-pyrido[4,3-b][1,4]oxazin-4-yl]pyridazin-3-yl]-3-methyl-phenol

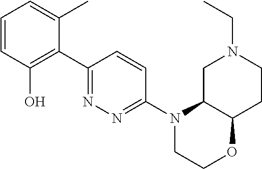 [SMILES: CCN1CC[C@H]2OCCN(c3ccc(-c4c(C)cccc4O)nn3)[C@H]2C1]

Under Ar, to a solution of (2-hydroxy-6-methyl-phenyl)boronic acid (Int 354.1-1.01 mmol, 153 mg) and (4aS,8aR)-4-(6-chloropyridazin-3-yl)-6-ethyl-3,4a,5,7,8,8a-hexahydro-2H-pyrido[4,3-b][1,4]oxazine (Int 313.1-0.67 mmol, 190 mg) in 20 ml of 1,4-dioxane were successively added a 2N sodium carbonate aqueous solution (1.81 mmol, 0.91 ml) and tetrakis(triphenylphosphine)palladium(0) (0.034 mmol, 39 mg). The mixture was stirred at 100° C. for 2 h. After cooling down to rt, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 95/2.4/2.4/0.2) to give the title compound as a white solid (58 mg, 24% yield).

### Example 402: 2-[6-[(4aS,8aR)-6-methyl-3,4a,5,7,8,8a-hexahydro-2H-pyrido[4,3-b][1,4]oxazin-4-yl]pyridazin-3-yl]-3-(trifluoromethyl)phenol

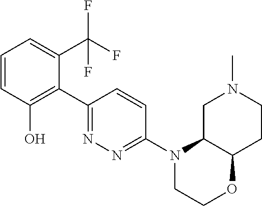 [SMILES: CN1CC[C@H]2OCCN(c3ccc(-c4c(O)cccc4C(F)(F)F)nn3)[C@H]2C1]

Under Ar, to a solution of [2-hydroxy-6-(trifluoromethyl)phenyl]boronic acid (0.41 mmol, 84 mg) and (4aS,8aR)-4-(6-chloropyridazin-3-yl)-6-methyl-3,4a,5,7,8,8a-hexahydro-2H-pyrido[4,3-b][1,4]oxazine (Int 321.3-0.23 mmol, 61 mg) in 3 ml of 1,4-dioxane were successively added a 2N sodium carbonate aqueous solution (0.61 mmol, 306 μl) and tetrakis (triphenylphosphine)palladium(0) (0.011 mmol, 13 mg). The mixture was stirred at 100° C. for 2 h30. After cooling down to rt, the mixture was diluted with 50 ml of EtOAc and 30 ml of water. The aqueous layer was extracted with a mixture of 35 ml of DCM and 15 ml of isopropanol and the combined organic layers were dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 95/2.4/2.4/0.2) to give the title compound as a pale yellow solid (74 mg, 82% yield).

### Example 398: 1-[(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone

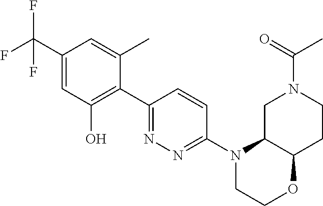 [SMILES: CC(=O)N1CC[C@H]2OCCN(c3ccc(-c4c(C)cc(C(F)(F)F)cc4O)nn3)[C@H]2C1]

### Int 398.1: 1-[(4aS,8aR)-4-(6-chloropyridazin-3-yl)-3,4a,5,7,8,8a-hexahydro-2H-pyrido [4,3-b][1,4]oxazin-6-yl]ethanone

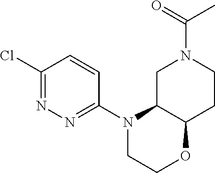 [SMILES: CC(=O)N1CC[C@H]2OCCN(c3ccc(Cl)nn3)[C@H]2C1]

To a solution of (4aS,8aR)-4-(6-chloropyridazin-3-yl)-2,3,4a,5,6,7,8,8a-octahydropyrido[4,3-b][1,4]oxazine (Int 321.2-5.69 mmol, 1.45 g), DMAP (0.285 mmol, 35 mg) and triethylamine (5.69 mmol, 780 μl) in 40 ml of DCM was added acetic anhydride (8.54 mmol, 802 μl). The mixture was stirred for 24 h at rt, then diluted with 100 ml of DCM. The organic layer was washed with 100 ml of a saturated aqueous sodium bicarbonate solution and 50 ml of water, then dried over Na₂SO₄, filtered and concentrated under reduced pressure. The residue was purified by flash chromatography on silica gel eluting with a gradient of 0 to 5% of MeOH in DCM to give the title compound as a yellow solid (1.69 g, 100% yield).

LCMS (Method V): Rt=0.63; MS m/z [M+H]⁺=297

### 1-[(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethenone (Example 398)

Under Ar, to a solution of 3-methyl-2-(4,4,5,5-tetramethyl-1,3,2-dioxaborolan-2-yl)-5-(trifluoromethyl)phenol (3.64 mmol, 1.1 g) and 1-[(4aS,8aR)-4-(6-chloropyridazin-3-yl)-3,4a,5,7,8,8a-hexahydro-2H-pyrido[4,3-b][1,4]oxazin-6-yl]ethanone (Int 398.1-2.02 mmol, 600 mg) in 20 ml of 1,4-dioxane were successively added a 2N sodium carbonate aqueous solution (5.46 mmol, 2.72 ml) and tetrakis(triphenylphosphine)palladium(0) (0.1 mmol, 117 mg). The mixture was stirred at 100° C. for 2 h. After cooling down to rt, the mixture was diluted with 200 ml of EtOAc and 200 ml of diethylether and the organic layer was washed successively with 2×150 ml of a saturated aqueous sodium bicarbonate solution and 100 ml of water. The organic layer was dried over Na₂SO₄, filtered and concentrated under reduced pressure. The residue was purified by flash chromatography on silica gel using a gradient of DCM/MeOH/ACN (from 100/0/0 to 95/2.5/2.5) to give the title compound as a yellow oil (506 mg, 57% yield).

Compounds Example 397, 409, 424, 462 and 463 were synthetized analogously to Example 398. Chiral separation of Examples 462 and 463 were realized using Chiralpak® IG column.

### Example 380: 1-[(4aS,8aR)-4-[6-[2-ethyl-6-hydroxy-4-(trifluoromethyl)phenyl]pyridazin-3-yl]-3,4a,5,7,8,8a-hexahydro-2H-pyrido[4,3-b][1,4]oxazin-6-yl]ethanone

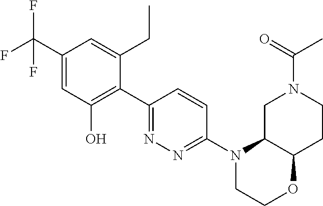 [SMILES: CCc1cc(C(F)(F)F)cc(O)c1-c1ccc(N2CCO[C@@H]3CCN(C(C)=O)C[C@@H]32)nn1]

Under Ar, to a solution of 3-ethyl-2-(4,4,5,5-tetramethyl-1,3,2-dioxaborolan-2-yl)-5-(trifluoromethyl)phenol (0.67 mmol, 213 mg) and 1-[(4aS,8aR)-4-(6-chloropyridazin-3-yl)-3,4a,5,7,8,8a-hexahydro-2H-pyrido[4,3-b][1,4]oxazin-6-yl]ethanone (Int 398.1-0.34 mmol, 100 mg) in 8 ml of 1,4-dioxane were successively added a 2N sodium carbonate aqueous solution (0.91 mmol, 455 μl) and tetrakis(triphenylphosphine)palladium(0) (0.017 mmol, 19 mg). The mixture was stirred at 100° C. for 1 h. After cooling down to rt, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 80/9.8/9.8/0.4) then repurified by prep-HPLC (column: Sunfire prep C18 OBD; 250*50 mm*10 μm; mobile phase: [aqueous TFA 10⁻² N/ACN] from 10% to 100% ACN in 35 min. at 50 ml/min) to give the title compound as a white solid (49 mg, 32% yield).

### Example 381: 1-[(4aS,8aR)-4-[6-(4-chloro-2-ethyl-6-hydroxy-phenyl)pyridazin-3-yl]-3,4a,5,7,8,8a-hexahydro-2H-pyrido[4,3-b][1,4]oxazin-6-yl]ethanone

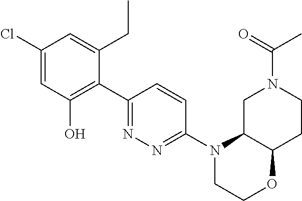 [SMILES: CCc1cc(Cl)cc(O)c1-c1ccc(N2CCO[C@@H]3CCN(C(C)=O)C[C@@H]32)nn1]

Under Ar, to a solution of 5-chloro-3-ethyl-2-(4,4,5,5-tetramethyl-1,3,2-dioxaborolan-2-yl)phenol (Int 381.3-0.67 mmol, 190 mg) and 1-[(4aS,8aR)-4-(6-chloropyridazin-3-yl)-3,4a,5,7,8,8a-hexahydro-2H-pyrido[4,3-b][1,4]oxazin-6-yl]ethanone (Int 398.1-0.34 mmol, 100 mg) in 8 ml of 1,4-dioxane were successively added a 2N sodium carbonate aqueous solution (0.91 mmol, 455 μl) and tetrakis(triphenylphosphine)palladium(0) (0.017 mmol, 19 mg). The mixture was stirred at 100° C. for 1 h. After cooling down to rt, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 80/9.8/9.8/0.4) then repurified by prep-HPLC (column: Sunfire prep C18 OBD; 250*50 mm*10 μm; mobile phase: [aqueous TFA 10⁻² N/ACN] from 10% to 100% ACN in 35 min. at 50 ml/min) to give the title compound as a white solid (53 mg, 38% yield).

### Example 304 and 397: 1-[(4aR,8aS)-4-[6-(4-chloro-2-hydroxy-6-methyl-phenyl)pyridazin-3-yl]-3,4a,5,7,8,8a-hexahydro-2H-pyrido[4,3-b][1,4]oxazin-6-yl]ethanone and 1-[(4aS,8aR)-4-[6-(4-chloro-2-hydroxy-6-methyl-phenyl)pyridazin-3-yl]-3,4a,5,7,8,8a-hexahydro-2H-pyrido[4,3-b][1,4]oxazin-6-yl]ethanone

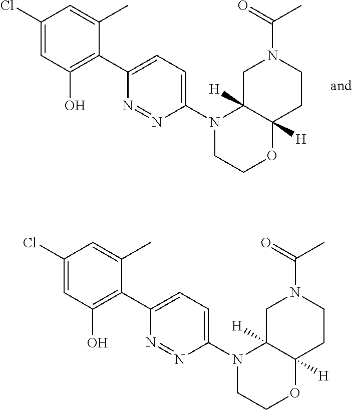 [SMILES: CC(=O)N1CC[C@@H]2OCCN(c3ccc(-c4c(C)cc(Cl)cc4O)nn3)[C@@H]2C1 ; CC(=O)N1CC[C@H]2OCCN(c3ccc(-c4c(C)cc(Cl)cc4O)nn3)[C@H]2C1]

1-[rac-(4aR,8aS)-4-[6-(4-chloro-2-hydroxy-6-methyl-phenyl)pyridazin-3-yl]-3,4a,5,7,8,8a-hexahydro-2H-pyrido[4,3-b][1,4]oxazin-6-yl]ethanone (Example 222-57 mg) was separated by Supercritical Fluidic Chromatography (SFC—Column: Daicel Chiralpak® AD, 300×50 mm I.D., 20 μm particle size; Mobile phase: CO₂/Methanol (+0.1% Triethylamine) 35%; flow rate: 250 mL/min; outlet pressure: 100 bar; 4 injections; UV detection: 265 nm) to provide the first enantiomer 1-[(4aS,8aR)-4-[6-(4-chloro-2-hydroxy-6-methyl-phenyl)pyridazin-3-yl]-3,4a,5,7,8,8a-hexahydro-2H-pyrido[4,3-b][1,4]oxazin-6-yl]ethanone eluting at Rt=23 min (7 mg, Example 397, Enantiomer 1), as a white solid, and the second enantiomer 1-[(4aR,8aS)-4-[6-(4-chloro-2-hydroxy-6-methyl-phenyl)pyridazin-3-yl]-3,4a,5,7,8,8a-hexahydro-2H-pyrido [4,3-b][1,4]oxazin-6-yl]ethanone, eluting at Rt=12.5 min (7 mg, Example 304, Enantiomer 2), as a white solid. The absolute configurations were assigned a posteriori (see Chiral synthesis of example 398).

### Example 307 and 321: 2-[6-[(4aR,8aS)-6-methyl-3,4a,5,7,8,8a-hexahydro-2H-pyrido[4,3-b][1,4]oxazin-4-yl]pyridazin-3-yl]-5-chloro-3-methyl-phenol and 2-[6-[(4aS,8aR)-6-methyl-3,4a,5,7,8,8a-hexahydro-2H-pyrido[4,3-b][1,4]oxazin-4-yl]pyridazin-3-yl]-5-chloro-3-methyl-phenol

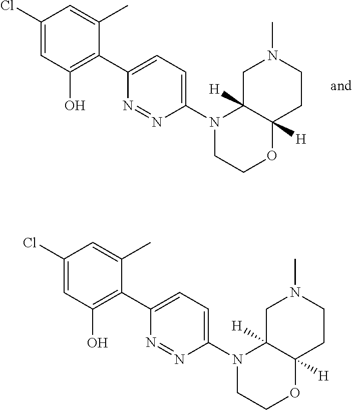 [SMILES: Cc1cc(Cl)cc(O)c1-c1ccc(N2CCO[C@H]3CCN(C)C[C@H]32)nn1 ; Cc1cc(Cl)cc(O)c1-c1ccc(N2CCO[C@@H]3CCN(C)C[C@@H]32)nn1]

5-chloro-3-methyl-2-[6-[rac-(4aR,8aS)-6-methyl-3,4a,5,7,8,8a-hexahydro-2H-pyrido[4,3-b][1,4]oxazin-4-yl]pyridazin-3-yl]phenol (Example 224-18 mg) was separated by Supercritical Fluidic Chromatography (SFC—Column: Daicel Chiralpak® AD, 300×50 mm I.D., 20 μm particle size; Mobile phase: CO₂/Methanol (+0.1% Triethylamine) 25%; flow rate: 250 mL/min; outlet pressure: 100 bar; 1 injections; UV detection: 265 nm) to provide the first enantiomer 2-[6-[(4aR,8aS)-6-methyl-3,4a,5,7,8,8a-hexahydro-2H-pyrido[4,3-b][1,4]oxazin-4-yl]pyridazin-3-yl]-5-chloro-3-methyl-phenol, eluting at Rt=5.37 min (8.4 mg, Example 307, Enantiomer 1), as a white solid, and the second enantiomer 2-[6-[(4aS,8aR)-6-methyl-3,4a,5,7,8,8a-hexahydro-2H-pyrido[4,3-b][1,4]oxazin-4-yl]pyridazin-3-yl]-5-chloro-3-methyl-phenol, eluting at Rt=8.84 min (9.1 mg, Example 321, Enantiomer 2), as a white solid. The absolute configurations were assigned a posteriori (see Chiral synthesis of example 321).

### Example 312 and 313: 2-[6-[(4aR,8aS)-6-ethyl-3,4a,5,7,8,8a-hexahydro-2H-pyrido[4,3-b][1,4]oxazin-4-yl]pyridazin-3-yl]-5-chloro-3-methyl-phenol and 2-[6-[(4aS,8aR)-6-ethyl-3,4a,5,7,8,8a-hexahydro-2H-pyrido[4,3-b][1,4]oxazin-4-yl]pyridazin-3-yl]-5-chloro-3-methyl-phenol

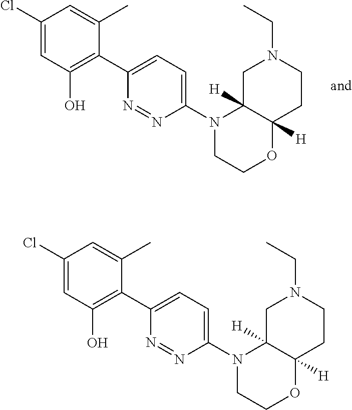 [SMILES: CCN1CC[C@@H]2OCCN(c3ccc(-c4c(C)cc(Cl)cc4O)nn3)[C@@H]2C1 ; CCN1CC[C@H]2OCCN(c3ccc(-c4c(C)cc(Cl)cc4O)nn3)[C@H]2C1]

5-chloro-3-methyl-2-[6-[rac-(4aR,8aS)-6-ethyl-3,4a,5,7,8,8a-hexahydro-2H-pyrido[4,3-b][1,4]oxazin-4-yl]pyridazin-3-yl]phenol (Example 223-53 mg) was separated by Supercritical Fluidic Chromatography (SFC—Column: Daicel Chiralpak® AD, 300×50 mm I.D., 20 μm particle size; Mobile phase: CO₂/Methanol (+0.1% Triethylamine) 25%; flow rate: 250 mL/min; outlet pressure: 100 bar; 1 injections; UV detection: 265 nm) to provide the first enantiomer 2-[6-[(4aR,8aS)-6-ethyl-3,4a,5,7,8,8a-hexahydro-2H-pyrido[4,3-b][1,4]oxazin-4-yl]pyridazin-3-yl]-5-chloro-3-methyl-phenol, first eluting enantiomer at Rt=4.7 min (22 mg, Example 312, Enantiomer 1), as a white solid, and the second enantiomer 2-[6-[(4aS,8aR)-6-ethyl-3,4a,5,7,8,8a-hexahydro-2H-pyrido[4,3-b][1,4]oxazin-4-yl]pyridazin-3-yl]-5-chloro-3-methyl-phenol, second eluting enantiomer at Rt=6.94 min (22 mg, Example 313, Enantiomer 2), as a white solid. The absolute configurations were assigned a posteriori (see Chiral synthesis of example 313).

### Example 344: 2-[6-[(4aR,8aR)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol

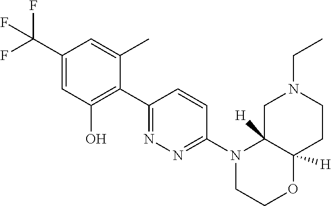 [SMILES: CCN1CC[C@H]2OCCN(c3ccc(-c4c(C)cc(C(F)(F)F)cc4O)nn3)[C@@H]2C1]

### Int 344.4: tert-butyl (4aR,8aR)-4-(6-chloropyridazin-3-yl)-3,4a,5,7,8,8a-hexahydro-2H-pyrido[4,3-b][1,4]oxazine-6-carboxylate

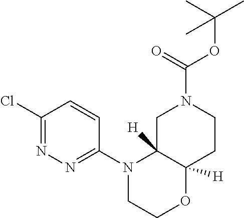 [SMILES: CC(C)(C)OC(=O)N1CC[C@H]2OCCN(c3ccc(Cl)nn3)[C@@H]2C1]

To a stirred solution of 3-bromo-6-chloro-pyridazine (2.05 g, 10.6 mmol) in 40 ml of 1,4-dioxane were added tert-butyl (4aR,8aR)-2,3,4,4a,5,7,8,8a-octahydropyrido[4,3-b][1,4]oxazine-6-carboxylate (Int 344.4-1.71 g, 7.06 mmol) and NaOtBu (2.03 g, 21.2 mmol). The mixture was purged with N₂. RuPhos Pd G3 (590 mg, 0.706 mmol) and Catacxium A (253 mg, 0.706 mmol) were then added, and the mixture was purged with N₂. The reaction mixture was stirred at 85° C. for 2 h. After cooling down to room temperature, the reaction mixture was filtered and concentrated under reduced pressure. The residue was purified by flash chromatography on silica gel using DCM/EtOAc (100/0 to 80/20) to give the title compound as a yellow oil (220 mg, 9%).

LCMS (Method T): Rt=6.08; MS m/z [M+H]⁺=355

### Int 344.5: (4aR,8aR)-4-(6-chloropyridazin-3-yl)-2,3,4a,5,6,7,8,8a-octahydropyrido[4,3-b][1,4]oxazine;2,2,2-trifluoroacetic acid

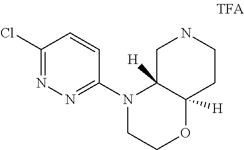 [SMILES: Clc1ccc(N2CCO[C@@H]3CCNC[C@H]32)nn1]

At 0° C., to a stirred solution of tert-butyl (4aR,8aR)-4-(6-chloropyridazin-3-yl)-3,4a,5,7,8,8a-hexahydro-2H-pyrido[4,3-b][1,4]oxazine-6-carboxylate (220 mg, 0.620 mmol) in 4 ml of dichloromethane was added TFA (0.461 mL, 6.20 mol) and the reaction was stirred at rt for 18 h. The mixture was concentrated under reduced pressure to afford the title compound as a yellow oil (296 mg) which was used without further purification in the next step.

LCMS (Method T): Rt=2.43; MS m/z [M+H]+=254.9

### Int 344.6: (4aR,8aR)-4-(6-chloropyridazin-3-yl)-6-ethyl-3,4a,5,7,8,8a-hexahydro-2H-pyrido[4,3-b][1,4]oxazine

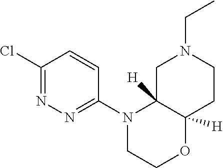 [SMILES: CCN1CC[C@H]2OCCN(c3ccc(Cl)nn3)[C@@H]2C1]

To a stirred solution of (4aR,8aR)-4-(6-chloropyridazin-3-yl)-2,3,4a,5,6,7,8,8a-octahydropyrido[4,3-b][1,4]oxazine;2,2,2-trifluoroacetic acid (155 mg, 0.321 mmol) in 5 ml of MeCN were added K₂CO₃ (443 mg, 3.21 mmol) and iodoethane (0.0258 mL, 0.321 mmol). The reaction mixture was stirred at 80° C. for 18 h. After cooling down to rt, the reaction mixture was filtered and concentrated under reduced pressure. The residue was purified by flash chromatography on silica gel using DCM/MeOH (100/0 to 80/20) to give the title compound as a yellow oil (46 mg, 51%).

LCMS (Method R): Rt=0.84; MS m/z [M+H]+=283.2

### 2-[6-[(4aR,8aR)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol (Example 344)

In a sealed tube, a mixture of (4aR,8aR)-4-(6-chloropyridazin-3-yl)-6-ethyl-3,4a,5,7,8,8a-hexahydro-2H-pyrido[4,3-b][1,4]oxazine (46.0 mg, 0.163 mmol), K₂CO₃ (67.4 mg, 0.488 mmol) and 3-methyl-2-(4,4,5,5-tetramethyl-1,3,2-dioxaborolan-2-yl)-5-(trifluoromethyl) phenol (73.7 mg, 0.244 mmol) in 1.6 ml of 1,4-dioxane and 0.4 ml of water was purged with N₂. XPhos Pd G2 (6.40 mg, 0.00813 mmol) and XPhos (3.88 mg, 0.00813 mmol) were added, the mixture was purged with N₂ and heated at 90° C. for 3 h. After cooling down to rt, water was added, and the resulting mixture was extracted twice with a 80/20 DCM/MeOH mixture. The organic layer was dried over MgSO₄, filtered and concentrated under reduced pressure. The residue was purified by flash chromatography on silica gel using DCM/MeOH (100/0 to 95/5) to give, after lyophilization, the title compound as a yellow oil (40 mg, 58%).

### Examples 336 and 349: 1-[(4aR,8aR)-4-[6-[2-hydroxy-6-methyl-4-(trifluoromethyl) phenyl]pyridazin-3-yl]-3,4a,5,7,8,8a-hexahydro-2H-pyrido[4,3-b][1,4]oxazin-6-yl]ethanone and 1-[(4aS,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4] oxazin-6-yl]ethanone

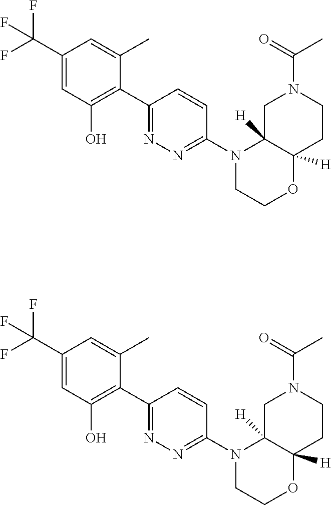 [SMILES: CC(=O)N1CC[C@H]2OCCN(c3ccc(-c4c(C)cc(C(F)(F)F)cc4O)nn3)[C@@H]2C1 ; CC(=O)N1CC[C@@H]2OCCN(c3ccc(-c4c(C)cc(C(F)(F)F)cc4O)nn3)[C@H]2C1]

1-[rac-(4aR,8aR)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone (75 mg), obtained similarly to ex 398 (with tert-butyl rac-(4aR,8aR)-2,3,4,4a,5,7,8,8a-octahydropyrido[4,3-b][1,4]oxazine-6-carboxylate instead of tert-butyl (4aS,8aR)-2,3,4,4a,5,7,8,8a-octahydropyrido[4,3-b][1,4]oxazine-6-carboxylate) was purified by preparative chiral SFC (Stationary phase: Chiralpak® AD-H 20×250 mm, Mobile phase: CO₂/iPrOH 80/20). The fractions containing each enantiomer were combined and concentrated under reduced pressure to give two residues. These two residues were respectively taken-up with MeCN, extended with water, then lyophilized overnight to give (22 mg, 12%) of a first enantiomer and (10 mg, 6%) of the second one. The absolute configurations cannot be assigned at this stage.

### Examples 301 and 343: 2-[6-[(4aR,8aR)-6-methyl-3,4a,5,7,8,8a-hexahydro-2H-pyrido[4,3-b][1,4]oxazin-4-yl]pyridazin-3-yl]-3-methyl-5-(trifluoromethyl)phenol and 2-[6-[(4aS,8aS)-6-methyl-3,4a,5,7,8,8a-hexahydro-2H-pyrido[4,3-b][1,4]oxazin-4-yl]pyridazin-3-yl]-3-methyl-5-(trifluoromethyl)phenol

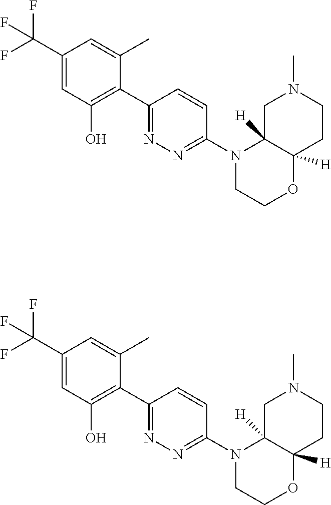 [SMILES: Cc1cc(C(F)(F)F)cc(O)c1-c1ccc(N2CCO[C@@H]3CCN(C)C[C@H]32)nn1 ; Cc1cc(C(F)(F)F)cc(O)c1-c1ccc(N2CCO[C@H]3CCN(C)C[C@@H]32)nn1]

2-[6-[rac-(4aR,8aR)-6-methyl-3,4a,5,7,8,8a-hexahydro-2H-pyrido[4,3-b][1,4]oxazin-4-yl]pyridazin-3-yl]-3-methyl-5-(trifluoromethyl)phenol (43 mg), obtained similarly to Example 358 (with tert-butyl rac-(4aR,8aR)-2,3,4,4a,5,7,8,8a-octahydropyrido[4,3-b][1,4]oxazine-6-carboxylate instead of tert-butyl (4aS,8aR)-2,3,4,4a,5,7,8,8a-octahydropyrido[4,3-b][1,4]oxazine-6-carboxylate) was purified by preparative chiral SFC (Stationary phase: Chiralpak® IC 20×250 mm, Mobile phase: CO₂/(iPrOH+0.3% iPrNH₂) 75/25). The fractions containing each enantiomer were combined and concentrated under reduced pressure to give two residues. These two residues were respectively taken-up with MeCN, extended with water, then lyophilized overnight to give (12 mg, 15%) of a first enantiomer and (15 mg, 19%) of the second one. The absolute configurations cannot be assigned at this stage.

### Example 358: 2-[6-[(4aS,8aR)-6-methyl-3,4a,5,7,8,8a-hexahydro-2H-pyrido[4,3-b][1,4]oxazin-4-yl]pyridazin-3-yl]-3-ethyl-phenol

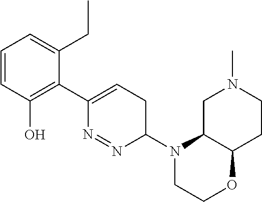 [SMILES: CCc1cccc(O)c1C1=CCC(N2CCO[C@@H]3CCN(C)C[C@@H]32)N=N1]

Under Ar, to a solution of (2-ethyl-6-hydroxy-phenyl)boronic acid (Int 358.2-1.49 mmol, 247 mg) and (4aS,8aR)-4-(6-chloropyridazin-3-yl)-6-methyl-3,4a,5,7,8,8a-hexahydro-2H-pyrido[4,3-b][1,4]oxazine (Int 321.3-0.74 mmol, 200 mg) in 8 ml of 1,4-dioxane were successively added a 2N sodium carbonate aqueous solution (2 mmol, 1 ml) and tetrakis (triphenylphosphine)palladium(0) (0.037 mmol, 43 mg). The mixture was stirred at 100° C. for 1 h. After cooling down to rt, the mixture was diluted with 50 ml of EtOAc and 50 ml of diethylether and the organic layer was washed successively with 2×50 ml of a saturated aqueous sodium bicarbonate solution and 50 ml of water. The organic layer was dried over Na₂SO₄, filtered, and concentrated under reduced pressure. The residue was purified by flash chromatography on silica gel using a gradient of DCM/MeOH/ACN/NH₃ 7N sol. in MeOH (from 100/0/0/0 to 90/4.9/4.9/0.2) to give the title compound as a white solid (125 mg, 47% yield).

Compounds Examples 353 and 354 were synthesized analogously to Example 358 starting from Int 353.2 and Int 354.1 respectively.

### Example 360: 2-[6-(5,6-dihydro-2H-pyrazolo[4,3-b][1,4]oxazin-7-yl)pyridazin-3-yl]-3-methyl-5-(trifluoromethyl)phenol

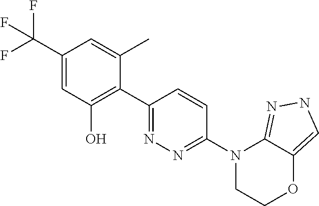 [SMILES: Cc1cc(C(F)(F)F)cc(O)c1-c1ccc(N2CCOc3c[nH]nc32)nn1]

### Int 360.7: 2-benzyl-7-(6-chloropyridazin-3-yl)-5,6-dihydropyrazolo[4,3-b][1,4]oxazine

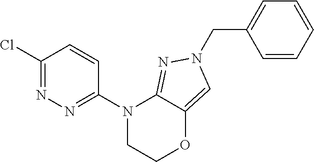 [SMILES: Clc1ccc(N2CCOc3cn(Cc4ccccc4)nc32)nn1]

In a sealed tube, a mixture of 3-bromo-6-chloro-pyridazine (0.178 g, 0.000920 mol), RuPhos Pd G3 (0.0321 g, 3.83e-5 mol), RuPhos (0.0358 g, 7.67e-5 mol) and NaOtBu (0.221 g, 0.00230 mol) was purged with N₂. 2-benzyl-6,7-dihydro-5H-pyrazolo[4,3-b][1,4]oxazine (0.165 g, 0.000767 mol) in 8 ml of 1,4-dioxane was added, the mixture was purged again with N₂ then heated at 100° C. for 2 h. After cooling down to rt, the mixture was concentrated under reduced pressure. The residue was purified by flash chromatography on silica gel using heptane/EtOAc (100/0 to 70/30) to give the title compound as an off-white solid (0.149 g, 59%).

LCMS (Method S): Rt=1.40; MS m/z [M+H]+=328.1

### Int 360.8: 2-[6-(2-benzyl-5,6-dihydropyrazolo[4,3-b][1,4]oxazin-7-yl)pyridazin-3-yl]-3-methyl-5-(trifluoromethyl)phenol

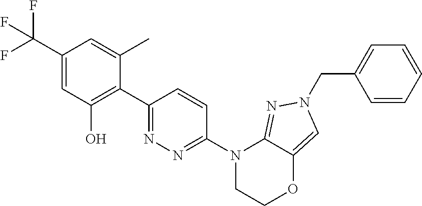 [SMILES: Cc1cc(C(F)(F)F)cc(O)c1-c1ccc(N2CCOc3cn(Cc4ccccc4)nc32)nn1]

In a sealed tube, a mixture of 2-benzyl-7-(6-chloropyridazin-3-yl)-5,6-dihydropyrazolo[4,3-b][1,4]oxazine (0.120 g, 0.000366 mol), 3-methyl-2-(4,4,5,5-tetramethyl-1,3,2-dioxaborolan-2-yl)-5-(trifluoromethyl)phenol (0.166 g, 0.000549 mol) and K₂CO₃ (0.101 g, 0.000732 mol) in 2.7 ml of 1,4-dioxane and 0.9 ml water was purged with N₂. XPhos Pd G2 (0.0144 g, 1.83e-5 mol) and XPhos (0.00873 g, 1.83e-5 mol) were added, the mixture was purged again with N₂ then heated at 90° C. for 18 h. After cooling down to rt, EtOAc and water were added. The aqueous layer was separated and extracted twice with EtOAc.

The combined organic layers were concentrated under reduced pressure, then coevaporated with toluene. The residue was purified by flash chromatography on silica gel using DCM to give 174 mg of pale yellow solid. This solid was triturated in Et₂O then filtered off to give the title compound as an off-white solid (0.149 g, 87%).

LCMS (Method E): Rt=3.45; MS m/z [M+H]+=468.4

### 2-[6-(5,6-dihydro-2H-pyrazolo[4,3-b][1,4]oxazin-7-yl)pyridazin-3-yl]-3-methyl-5-(trifluoromethyl)phenol (Example 360)

Under N₂, DMSO (0.152 mL, 0.00214 mol) was added to 2-[6-(2-benzyl-5,6-dihydropyrazolo[4,3-b][1,4]oxazin-7-yl)pyridazin-3-yl]-3-methyl-5-(trifluoromethyl) phenol (0.100 g, 0.214 mmol). tBuOK 1M in THF (1.00 mol/L, 1.50 mL, 0.00150 mol) was added and the mixture was purged with 02 and stirred at rt for 18 h. The solution was diluted with EtOAc and slowly neutralized with a 1N aqueous solution of HCl. The aqueous layer was separated and extracted twice with EtOAc. The combined organic layers were dried over MgSO₄, filtered and concentrated under reduced pressure. The residue was purified by flash chromatography on silica gel using DCM/MeOH (100/0 to 90/10) to give 42 mg of a yellow solid. This solid was purified by flash chromatography on silica gel using heptane/EtOAc (100/0 to 50/50) to give the title compound as a pale yellow solid (20 mg, 25%).

### Example 373: 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-methyl-phenol

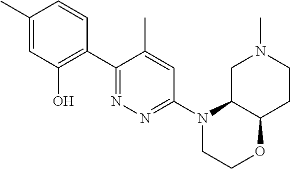 [SMILES: Cc1ccc(-c2nnc(N3CCO[C@@H]4CCN(C)C[C@@H]43)cc2C)c(O)c1]

### Int 373.1 and Int 434.1: tert-butyl (4aS,8aR)-4-(6-chloro-5-methyl-pyridazin-3-yl)-3,4a,5,7,8,8a-hexahydro-2H-pyrido[4,3-b][1,4]oxazine-6-carboxylate (Int 373.1) and tert-butyl (4aS,8aR)-4-(6-chloro-4-methyl-pyridazin-3-yl)-3,4a,5,7,8,8a-hexahydro-2H-pyrido[4,3-b][1,4]oxazine-6-carboxylate (Int 434.1)

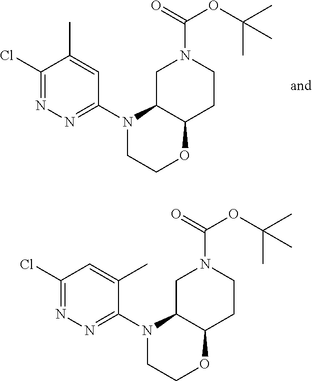

To a mixture of tert-butyl (4aS,8aR)-2,3,4,4a,5,7,8,8a-octahydropyrido[4,3-b][1,4]oxazine-6-carboxylate (Int 298.3-3.30 mmol, 800 mg) and 3,6-dichloro-4-methyl-pyridazine (4.95 mmol, 807 mg was added triethylamine (6.6 mmol, 928 μl). The mixture was stirred at 150° C. for 10 min under a flow of argon, then stirred at 150° C. for 5 h. The neat solution was cooled to rt, diluted with 10 ml of DCM and purified by flash chromatography on silica gel eluting with a gradient of 0 to 60% of EtOAc in cyclohexane to give successively tert-butyl (4aS,8aR)-4-(6-chloro-4-methyl-pyridazin-3-yl)-3,4a,5,7,8,8a-hexahydro-2H-pyrido[4,3-b][1,4]oxazine-6-carboxylate (Int 434.1) (50 mg, 4% yield) and tert-butyl (4aS,8aR)-4-(6-chloro-5-methyl-pyridazin-3-yl)-3,4a,5,7,8,8a-hexahydro-2H-pyrido[4,3-b][1,4]oxazine-6-carboxylate (Int 373.1) (460 mg, 38% yield) as yellow oils.

### LCMS (Method V)

Int 434.1: Rt=0.97; MS m/z [M+H]⁺=369
Int 373.1: Rt=0.98; MS m/z [M+H]⁺=369

### Int 373.2: (4aS,8aR)-4-(6-chloro-5-methyl-pyridazin-3-yl)-2,3,4a,5,6,7,8,8a-octahydropyrido[4,3-b][1,4]oxazine

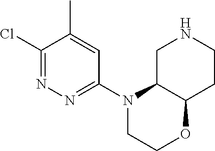 [SMILES: Cc1cc(N2CCO[C@@H]3CCNC[C@@H]32)nnc1Cl]

A solution of tert-butyl (4aS,8aR)-4-(6-chloro-5-methyl-pyridazin-3-yl)-3,4a,5,7,8,8a-hexahydro-2H-pyrido[4,3-b][1,4]oxazine-6-carboxylate (1.21 mmol, 445 mg) in 18 ml of dichloromethane and 2 ml of trifluoroacetic acid was stirred at rt for 4 h, then concentrated under reduced pressure to give the title compound (324 mg, 100% yield) as a brown oil.

LCMS (Method V): Rt=0.33; MS m/z [M+H]⁺=269

### Int 373.3: (4aS,8aR)-4-(6-chloro-5-methyl-pyridazin-3-yl)-6-methyl-3,4a,5,7,8,8a-hexahydro-2H-pyrido[4,3-b][1,4]oxazine

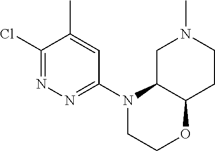 [SMILES: Cc1cc(N2CCO[C@@H]3CCN(C)C[C@@H]32)nnc1Cl]

Under argon, to a solution of (4aS,8aR)-4-(6-chloro-5-methyl-pyridazin-3-yl)-2,3,4a,5,6,7,8,8a-octahydropyrido[4,3-b][1,4]oxazine (0.89 mmol, 240 mg) in 10 ml of DCM were successively added 767 μl of acetic acid, a 37% aqueous solution of formaldehyde (4.46 mmol, 362 mg) and sodium triacetoxyborohydride (1.6 mmol, 341 mg). The mixture was stirred at rt for 18 h, then diluted with 20 ml of dichloromethane and 20 ml of a saturated aqueous sodium bicarbonate solution. The organic layer was dried over Na₂SO₄, filtered and concentrated under reduced pressure. The residue was purified by flash chromatography on silica gel using a gradient of DCM/MeOH/ACN (from 100/0/0 to 90/5/5) to give the title compound as a yellow oil (214 mg, 85% yield).

LCMS (Method V): Rt=0.34; MS m/z [M+H]⁺=283

### 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-methyl-phenol (Example 373)

Under Ar, to a solution of 5-methyl-2-(4,4,5,5-tetramethyl-1,3,2-dioxaborolan-2-yl)phenol (0.53 mmol, 124 mg) and (4aS,8aR)-4-(6-chloro-5-methyl-pyridazin-3-yl)-6-methyl-3,4a,5,7,8,8a-hexahydro-2H-pyrido[4,3-b][1,4]oxazine (0.35 mmol, 100 mg) in 8 ml of 1,4-dioxane was added a 2N sodium carbonate aqueous solution (0.95 mmol, 477 μl). tetrakis(triphenylphosphine)palladium(0) (0.018 mmol, 20 mg) was added and the solution was stirred at 100° C. for 1 h. After cooling down to rt, the mixture was diluted with 50 ml of EtOAc and 50 ml of diethylether and the organic layer was washed successively with 20 ml of a saturated aqueous sodium bicarbonate solution (twice) and 20 ml of water. The organic layer was dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 80/9.8/9.8/0.4) to give the title compound as a white solid (23 mg, 18% yield).

Compound Example 356 was synthetized analogously to Example 373 Compounds Examples 403, 421, 438 and 448 were synthesized starting from dichloropyridazine with conditions for N-arylation and boronate coupling analogously to Example 373.

Compounds Examples 443 and 470 were synthesized analogously to Example 373, starting from 3,6-dichloro-4-ethyl-pyridazine.

### Example 434: 2-[6-[(4aS,8aR)-6-methyl-3,4a,5,7,8,8a-hexahydro-2H-pyrido[4,3-b][1,4]oxazin-4-yl]-5-methyl-pyridazin-3-yl]-3,5-dimethyl-phenol

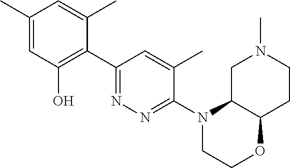 [SMILES: Cc1cc(C)c(-c2cc(C)c(N3CCO[C@@H]4CCN(C)C[C@@H]43)nn2)c(O)c1]

### Int 434.2: (4aS,8aR)-4-(6-chloro-4-methyl-pyridazin-3-yl)-6-methyl-3,4a,5,7,8,8a-hexahydro-2H-pyrido[4,3-b][1,4]oxazine

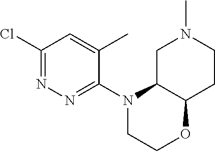 [SMILES: Cc1cc(Cl)nnc1N1CCO[C@@H]2CCN(C)C[C@@H]21]

Following procedures of Int 373.2 and Int 373.3, tert-butyl (4aS,8aR)-4-(6-chloro-4-methyl-pyridazin-3-yl)-3,4a,5,7,8,8a-hexahydro-2H-pyrido[4,3-b][1,4]oxazine-6-carboxylate (Int 434.1-0.136 mmol, 50 mg) give (4aS,8aR)-4-(6-chloro-4-methyl-pyridazin-3-yl)-6-methyl-3,4a,5,7,8,8a-hexahydro-2H-pyrido[4,3-b][1,4]oxazine (27 mg, 70% yield) as a yellow oil.

LCMS (Method V): Rt=0.68; MS m/z [M+H]⁺=283

### 2-[6-[(4aS,8aR)-6-methyl-3,4a,5,7,8,8a-hexahydro-2H-pyrido[4,3-b][1,4]oxazin-4-yl]-5-methyl-pyridazin-3-yl]-3,5-dimethyl-phenol (Example 434)

Under Ar, to a solution of (2-hydroxy-4,6-dimethyl-phenyl)boronic acid (0.19 mmol, 32 mg) and (4aS,8aR)-4-(6-chloro-4-methyl-pyridazin-3-yl)-6-methyl-3,4a,5,7,8,8a-hexahydro-2H-pyrido[4,3-b][1,4]oxazine (0.096 mmol, 27 mg) in 1 ml of 1,4-dioxane were added a 2N sodium carbonate aqueous solution (0.29 mmol, 143 μl) and tetrakis(triphenylphosphine)palladium(0) (0.01 mmol, 11 mg). The solution was stirred at 90° C. for 4 h. After cooling down, the mixture was diluted with 20 ml of DCM and 5 ml of a saturated aqueous sodium bicarbonate solution and the aqueous layer was extracted with 5 ml of DCM. The combined organic layers were dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 90/4.9/4.9/0.2) to give the title compound as a white solid (23 mg, 65% yield).

Compounds Examples 439 was synthesized analogously to Example 434.

### Example 430: 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-(trifluoromethyl)phenol

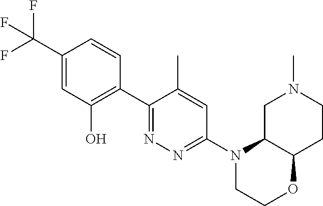 [SMILES: Cc1cc(N2CCO[C@@H]3CCN(C)C[C@@H]32)nnc1-c1ccc(C(F)(F)F)cc1O]

Under Ar, to a solution of [2-hydroxy-4-(trifluoromethyl)phenyl]boronic acid (1.36 mmol, 281 mg) and (4aS,8aR)-4-(6-chloro-5-methyl-pyridazin-3-yl)-6-methyl-3,4a,5,7,8,8a-hexahydro-2H-pyrido[4,3-b][1,4]oxazine (Int 373.3-0.76 mmol, 214 mg) in 8 ml of 1,4-dioxane were added a 2N sodium carbonate aqueous solution (2.04 mmol, 1.02 ml) and tetrakis(triphenylphosphine)palladium(0) (0.038 mmol, 44 mg). The mixture was stirred at 100° C. for 2 h. After cooling down, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered, and concentrated under reduced pressure. The residue was purified by flash chromatography on silica gel using a gradient of DCM/MeOH/ACN (from 100/0/0 to 90/5/5) to give a solid that was triturated in 2 ml of acetonitrile to give the title compound as a white solid (60 mg, 19% yield).

### Example 379: 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-chloro-phenol

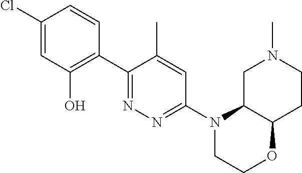 [SMILES: Cc1cc(N2CCO[C@@H]3CCN(C)C[C@@H]32)nnc1-c1ccc(Cl)cc1O]

Under Ar, to a solution of 5-chloro-2-(4,4,5,5-tetramethyl-1,3,2-dioxaborolan-2-yl)phenol (0.53 mmol, 135 mg) and (4aS,8aR)-4-(6-chloro-5-methyl-pyridazin-3-yl)-6-methyl-3,4a,5,7,8,8a-hexahydro-2H-pyrido[4,3-b][1,4]oxazine (Int 373.3-0.35 mmol, 100 mg) in 8 ml of 1,4-dioxane was added a 2N sodium carbonate aqueous solution (0.91 mmol, 455 μl). tetrakis(triphenylphosphine)palladium(0) (0.018 mmol, 20 mg) was then added and the mixture was stirred at 100° C. for 1 h. After cooling down to rt, the mixture was diluted with 50 ml of EtOAc and 50 ml of diethylether and the organic layer was washed successively with 20 ml of a saturated aqueous sodium bicarbonate solution (twice) and 20 ml of water. The organic layer was dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 90/4.9/4.9/0.2), then repurified by prep-HPLC (column: Sunfire prep C18 OBD; 250*50 mm*10 μm; mobile phase: [aqueous TFA 10⁻² N/ACN] from 10% to 100% ACN in 35 min. at 50 ml/min) to give the title compound as a white solid (25 mg, 19% yield).

### Example 363: 1-[(4aS,8aR)-4-[6-[2-hydroxy-4-(trifluoromethyl)phenyl]-5-methyl-pyridazin-3-yl]-3,4a,5,7,8,8a-hexahydro-2H-pyrido[4,3-b][1,4]oxazin-6-yl]ethanone

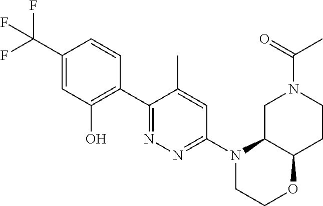 [SMILES: CC(=O)N1CC[C@H]2OCCN(c3cc(C)c(-c4ccc(C(F)(F)F)cc4O)nn3)[C@H]2C1]

### Int 363.1: 1-[(4aS,8aR)-4-(6-chloro-5-methyl-pyridazin-3-yl)-3,4a,5,7,8,8a-hexahydro-2H-pyrido[4,3-b][1,4]oxazin-6-yl]ethanone

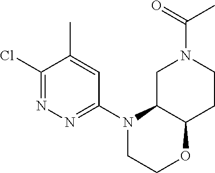 [SMILES: CC(=O)N1CC[C@H]2OCCN(c3cc(C)c(Cl)nn3)[C@H]2C1]

To a solution of (4aS,8aR)-4-(6-chloro-5-methyl-pyridazin-3-yl)-2,3,4a,5,6,7,8,8a-octahydropyrido[4,3-b][1,4]oxazine (Int 373.2-0.89 mmol, 240 mg), DMAP (0.045 mmol, 5.5 mg) and triethylamine (0.89 mmol, 120 μl) in 10 ml of DCM was added acetic anhydride (1.34 mmol, 126 μl). The mixture was stirred for 24 h at rt. The mixture was then diluted with 50 ml of DCM. The organic layer was washed with 50 ml of a saturated aqueous sodium bicarbonate solution and 20 ml of water, then dried over Na₂SO₄, filtered and concentrated under reduced pressure. The residue was purified by flash chromatography on silica gel eluting with a gradient of 0 to 5% of MeOH in DCM to give the title compound as a yellow solid (269 g, 97% yield).

LCMS (Method V): Rt=0.64; MS m/z [M+H]⁺=311

### 1-[(4aS,8aR)-4-[6-[2-hydroxy-4-(trifluoromethyl)phenyl]-5-methyl-pyridazin-3-yl]-3,4a,5,7,8,8a-hexahydro-2H-pyrido[4,3-b][1,4]oxazin-6-yl]ethenone (Example 363)

Under Ar, to a solution of [2-hydroxy-4-(trifluoromethyl)phenyl]boronic acid (1.29 mmol, 265 mg) and 1-[(4aS,8aR)-4-(6-chloro-5-methyl-pyridazin-3-yl)-3,4a,5,7,8,8a-hexahydro-2H-pyrido[4,3-b][1,4]oxazin-6-yl]ethanone (0.64 mmol, 200 mg) in 8 ml of 1,4-dioxane was successively added a 2N sodium carbonate aqueous solution (1.74 mmol, 869 μl) and tetrakis(triphenylphosphine)palladium(0) (0.032 mmol, 37 mg). The mixture was stirred at 100° C. for 1 h. After cooling down to rt, the mixture was diluted with 50 ml of EtOAc and 50 ml of diethylether and the organic layer was washed successively with 20 ml of a saturated aqueous sodium bicarbonate solution (twice) and 20 ml of water. The organic layer was dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 80/9.8/9.8/0.4) to give the title compound as a white solid (59 mg, 21% yield).

Compounds Example 357 and 431 were synthetized analogously to Example 363

### Example 405: 1-[(4aS,8aR)-4-[4-(difluoromethyl)-6-(2-hydroxy-4,6-dimethyl-phenyl)pyridazin-3-yl]-3,4a,5,7,8,8a-hexahydro-2H-pyrido[4,3-b][1,4]oxazin-6-yl]ethanone

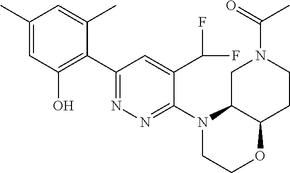 [SMILES: CC(=O)N1CC[C@H]2OCCN(c3nnc(-c4c(C)cc(C)cc4O)cc3C(F)F)[C@H]2C1]

### Int 405.1: 1-[(4aS,8aR)-4-[6-chloro-4-(difluoromethyl)pyridazin-3-yl]-3,4a,5,7,8,8a-hexahydro-2H-pyrido[4,3-b][1,4]oxazin-6-yl]ethanone

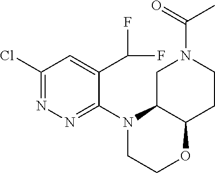 [SMILES: CC(=O)N1CC[C@H]2OCCN(c3nnc(Cl)cc3C(F)F)[C@H]2C1]

Following procedure of Int 386.1, (4aS,8aR)-4-[6-chloro-4-(difluoromethyl)pyridazin-3-yl]-2,3,4a,5,6,7,8,8a-octahydropyrido[4,3-b][1,4]oxazine (Int 291.2-1.31 mmol, 400 mg) gave (1-[(4aS,8aR)-4-[6-chloro-4-(difluoromethyl)pyridazin-3-yl]-3,4a,5,7,8,8a-hexahydro-2H-pyrido[4,3-b][1,4]oxazin-6-yl]ethanone (410 mg, 90% yield) as a yellow oil.

LCMS (Method V): Rt=0.79; MS m/z [M+H]⁺=347

### 1-[(4aS,8aR)-4-[4-(difluoromethyl)-6-(2-hydroxy-4,6-dimethyl-phenyl)pyridazin-3-yl]-3,4a,5,7,8,8a-hexahydro-2H-pyrido[4,3-b][1,4]oxazin-6-yl]ethenone (Example 405)

Following procedure of Example 291, 1-[(4aS,8aR)-4-[6-chloro-4-(difluoromethyl)pyridazin-3-yl]-3,4a,5,7,8,8a-hexahydro-2H-pyrido[4,3-b][1,4]oxazin-6-yl]ethanone (0.63 mmol, 220 mg) gave 1-[(4aS,8aR)-4-[4-(difluoromethyl)-6-(2-hydroxy-4,6-dimethyl-phenyl)pyridazin-3-yl]-3,4a,5,7,8,8a-hexahydro-2H-pyrido[4,3-b][1,4]oxazin-6-yl]ethanone (225 mg, 82% yield) as a yellow oil.

### Example 432: 2-[6-[(4aS,8aR)-6-isopropyl-3,4a,5,7,8,8a-hexahydro-2H-pyrido[4,3-b][1,4]oxazin-4-yl]pyridazin-3-yl]-3-ethyl-phenol

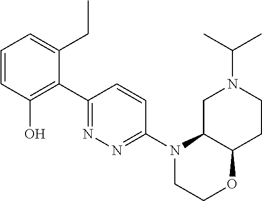 [SMILES: CCc1cccc(O)c1-c1ccc(N2CCO[C@@H]3CCN(C(C)C)C[C@@H]32)nn1]

### Int 432.1: (4aS,8aR)-4-(6-chloropyridazin-3-yl)-6-isopropyl-3,4a,5,7,8,8a-hexahydro-2H-pyrido[4,3-b][1,4]oxazine

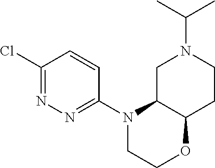 [SMILES: CC(C)N1CC[C@H]2OCCN(c3ccc(Cl)nn3)[C@H]2C1]

To a solution of (4aS,8aR)-4-(6-chloropyridazin-3-yl)-2,3,4a,5,6,7,8,8a-octahydropyrido[4,3-b][1,4]oxazine (Int 321.2-1.69 mmol, 430 mg) in 20 ml of dichloromethane were added successively 1.45 ml of acetic acid, acetone (8.44 mmol, 625 μl) and sodium triacetoxyborohydride (3.04 mmol, 644 mg) portionwise. The mixture was stirred at rt for 18 h, heated to 40° C. for 2 h then diluted with 100 ml of dichloromethane and 100 ml of a saturated aqueous sodium bicarbonate solution. The aqueous layer was extracted with 4×100 ml of a mixture DCM/isopropanol: 90/10 and the combined organic layers were dried over Na₂SO₄, filtered and concentrated under reduced pressure. The residue was solubilized in 20 ml of dichloromethane and then were added again 1.5 ml of acetic acid, acetone (8.44 mmol, 625 μl) and sodium triacetoxyborohydride (3.04 mmol, 644 mg) portion wise. The mixture was stirred at rt for 18 h. Acetone (6.75 mmol, 500 μL) and sodium triacetoxyborohydride (1.69 mmol, 358 mg) were added and the mixture was stirred at rt for 3 h, then diluted with 100 ml of dichloromethane and 100 ml of a saturated aqueous sodium bicarbonate solution. The organic layer was dried over Na₂SO₄, filtered, and concentrated under reduced pressure. The residue was purified by flash chromatography on silica gel using a gradient of DCM/MeOH/ACN (from 100/0/0 to 90/5/5) to give the title compound as a yellow oil (425 mg, 85% yield).

LCMS (Method V): Rt=0.58; MS m/z [M+H]⁺=297

### 2-[6-[(4aS,8aR)-6-isopropyl-3,4a,5,7,8,8a-hexahydro-2H-pyrido[4,3-b][1,4]oxazin-4-yl] pyridazin-3-yl]-3-ethyl-phenol (Example 432)

Under Ar, to a solution of (2-ethyl-6-hydroxy-phenyl)boronic acid (Int 358.2-1.27 mmol, 211 mg) and (4aS,8aR)-4-(6-chloropyridazin-3-yl)-6-isopropyl-3,4a,5,7,8,8a-hexahydro-2H-pyrido[4,3-b][1,4]oxazine (0.71 mmol, 210 mg) in 8 ml of 1,4-dioxane were added a 2N sodium carbonate aqueous solution (1.91 mmol, 955 μl) and tetrakis(triphenylphosphine)palladium(0) (0.035 mmol, 41 mg). The mixture was stirred at 100° C. for 2 h. After cooling down, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered and concentrated under reduced pressure. The residue was purified by flash chromatography on silica gel using a gradient of DCM/MeOH/ACN (from 100/0/0 to 95/2.5/2.5) to give the title compound as a white solid (63 mg, 23% yield).

### Example 433: 2-[6-[(4aS,8aR)-6-isopropyl-3,4a,5,7,8,8a-hexahydro-2H-pyrido[4,3-b][1,4]oxazin-4-yl]pyridazin-3-yl]-3,5-dimethyl-phenol

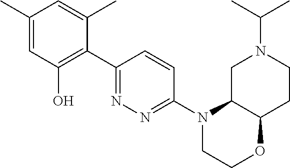 [SMILES: Cc1cc(C)c(-c2ccc(N3CCO[C@@H]4CCN(C(C)C)C[C@@H]43)nn2)c(O)c1]

Under Ar, to a solution of (2-hydroxy-4,6-dimethyl-phenyl)boronic acid (1.27 mmol, 211 mg) and (4aS,8aR)-4-(6-chloropyridazin-3-yl)-6-isopropyl-3,4a,5,7,8,8a-hexahydro-2H-pyrido[4,3-b][1,4]oxazine (Int 432.1-0.71 mmol, 210 mg) in 8 ml of 1,4-dioxane were added a 2N sodium carbonate aqueous solution (1.91 mmol, 955 μl) and tetrakis(triphenylphosphine)palladium(0) (0.035 mmol, 41 mg). The mixture was stirred at 100° C. for 2 h. After cooling down, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered and concentrated under reduced pressure. The residue was purified by flash chromatography on silica gel using a gradient of DCM/MeOH/ACN (from 100/0/0 to 95/2.5/2.5) to give the title compound as a white solid (135 mg, 50% yield).

### Example 411: 2-[6-[(4aS,8aR)-2,3,4a,5,6,7,8,8a-octahydropyrido[4,3-b][1,4]oxazin-4-yl]pyridazin-3-yl]-3-methyl-phenol

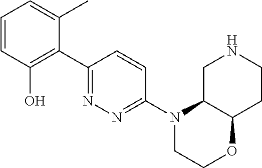 [SMILES: Cc1cccc(O)c1-c1ccc(N2CCO[C@@H]3CCNC[C@@H]32)nn1]

Following procedures of Int 322.1 and Example 322, tert-butyl (4aS,8aR)-4-(6-chloropyridazin-3-yl)-3,4a,5,7,8,8a-hexahydro-2H-pyrido[4,3-b][1,4]oxazine-6-carboxylate (Int 321.1-0.42 mmol, 150 mg) gave 2-[6-[(4aS,8aR)-2,3,4a,5,6,7,8,8a-octahydropyrido[4,3-b][1,4]oxazin-4-yl]pyridazin-3-yl]-3-methyl-phenol (410 mg, 45% yield) as a white solid.

### Example 435: 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-methyl-phenol

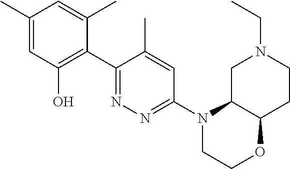 [SMILES: CCN1CC[C@H]2OCCN(c3cc(C)c(-c4c(C)cc(C)cc4O)nn3)[C@H]2C1]

### Int 435.1: (4aS,8aR)-4-(6-chloro-5-methyl-pyridazin-3-yl)-6-ethyl-3,4a,5,7,8,8a-hexahydro-2H-pyrido[4,3-b][1,4]oxazine

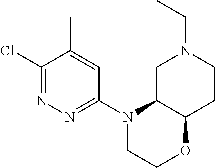 [SMILES: CCN1CC[C@H]2OCCN(c3cc(C)c(Cl)nn3)[C@H]2C1]

Under argon, to a solution of (4aS,8aR)-4-(6-chloro-5-methyl-pyridazin-3-yl)-2,3,4a,5,6,7,8,8a-octahydropyrido[4,3-b][1,4]oxazine (Int 373.2-1.21 mmol, 324 mg) in 10 ml of DCM were successively added 1.03 ml of acetic acid, acetaldehyde (6.03 mmol, 337 μl) and sodium triacetoxyborohydride (2.17 mmol, 460 mg). The mixture was stirred at rt for 18 h then diluted with 20 ml of dichloromethane and 20 ml of a saturated aqueous sodium bicarbonate solution. The aqueous layer was extracted with 3×50 ml of DCM and 2×50 ml of a mixture of DCM/isopropanol: 90/10. The combined organic layers were dried over Na₂SO₄, filtered, and concentrated under reduced pressure. The residue was purified by flash chromatography on silica gel using a gradient of DCM/MeOH/ACN (from 100/0/0 to 80/10/10) to give the title compound as a yellow foam (300 mg, 84% yield).

LCMS (Method V): Rt=0.35; [M+H]⁺=297

### 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-methyl-phenol (Example 435)

Under Ar, to a solution of 5-methyl-2-(4,4,5,5-tetramethyl-1,3,2-dioxaborolan-2-yl)phenol (0.51 mmol, 118 mg) and (4aS,8aR)-4-(6-chloro-5-methyl-pyridazin-3-yl)-6-ethyl-3,4a,5,7,8,8a-hexahydro-2H-pyrido[4,3-b][1,4]oxazine (0.34 mmol, 100 mg) in 4 ml of 1,4-dioxane were added a 2N sodium carbonate aqueous solution (0.91 mmol, 455 μl) and tetrakis(triphenylphosphine)palladium(0) (0.017 mmol, 19 mg). The mixture was stirred at 100° C. for 1 h. After cooling down, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered and concentrated under reduced pressure. The residue was purified by flash chromatography on silica gel using a gradient of DCM/MeOH/ACN (from 100/0/0 to 85/7.5/7.5) to give the title compound as a white solid (62 mg, 50% yield).

### Example 436: 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-(trifluoromethyl)phenol

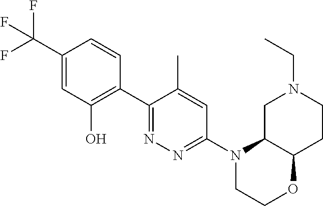 [SMILES: CCN1CC[C@H]2OCCN(c3cc(C)c(-c4ccc(C(F)(F)F)cc4O)nn3)[C@H]2C1]

Under Ar, to a solution of [2-hydroxy-4-(trifluoromethyl)phenyl]boronic acid (0.51 mmol, 104 mg) and (4aS,8aR)-4-(6-chloro-5-methyl-pyridazin-3-yl)-6-ethyl-3,4a,5,7,8,8a-hexahydro-2H-pyrido[4,3-b][1,4]oxazine (Int 435.1-0.34 mmol, 100 mg) in 4 ml of 1,4-dioxane were added a 2N sodium carbonate aqueous solution (0.91 mmol, 455 μl) and tetrakis(triphenylphosphine)palladium(0) (0.017 mmol, 19 mg). The mixture was stirred at 100° C. for 1 h. After cooling down, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered, and concentrated under reduced pressure. The residue was purified by flash chromatography on silica gel using a gradient of DCM/MeOH/ACN (from 100/0/0 to 85/7.5/7.5) to give the title compound as a white solid (43 mg, 30% yield).

### Example 437: 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-chloro-phenol

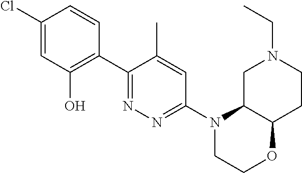 [SMILES: CCN1CC[C@H]2OCCN(c3cc(C)c(-c4ccc(Cl)cc4O)nn3)[C@H]2C1]

Under Ar, to a solution of (4-chloro-2-hydroxy-phenyl)boronic acid (0.51 mmol, 87 mg) and (4aS,8aR)-4-(6-chloro-5-methyl-pyridazin-3-yl)-6-ethyl-3,4a,5,7,8,8a-hexahydro-2H-pyrido [4,3-b][1,4]oxazine (Int 435.1-0.34 mmol, 100 mg) in 4 ml of 1,4-dioxane were added a 2N sodium carbonate aqueous solution (0.91 mmol, 455 μl) and tetrakis(triphenylphosphine)palladium(0) (0.017 mmol, 19 mg). The mixture was stirred at 100° C. for 1 h. After cooling down, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered, and concentrated under reduced pressure. The residue was purified by flash chromatography on silica gel using a gradient of DCM/MeOH/ACN (from 100/0/0 to 85/7.5/7.5) and the obtained solid was triturated in 2 ml of acetonitrile to give the title compound as a white solid (15 mg, 110% yield).

### Example 300: rac-(4aR,8aR)-1-[6-(2-hydroxy-4,6-dimethyl-phenyl)pyridazin-3-yl]-2,3,4,4a,5,6,7,8a-octahydro-1,7-naphthyridin-8-one

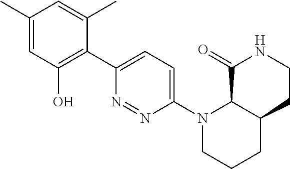 [SMILES: Cc1cc(C)c(-c2ccc(N3CCC[C@@H]4CCNC(=O)[C@@H]43)nn2)c(O)c1]

### Int 300.1: rac-(4aR,8aR)-1-(6-chloropyridazin-3-yl)-2,3,4,4a,5,6,7,8a-octahydro-1,7-naphthyridin-8-one

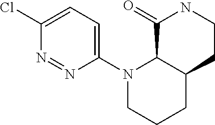 [SMILES: O=C1NCC[C@H]2CCCN(c3ccc(Cl)nn3)[C@@H]12]

A solution of 2,3,4,4a,5,6,7,8a-octahydro-1H-1,7-naphthyridin-8-one (5.19 mmol, 800 mg), 3,6-dichloropyridazine (6.22 mmol, 927 mg) and triethylamine (72.6 mmol, 10.1 ml) in 30 ml of 1-butanol was stirred at reflux for 6 h, then for 1 h while removing 1-butanol and triethylamine under a stream of argon. The neat mixture was cooled down to 50° C. Another 4.5 ml of triethylamine and 20 ml of 1-butanol were added, and the mixture was stirred at 160° C. for another 2 h while removing 1-butanol and triethylamine under a stream of argon. The operation was re-iterated twice. The neat mixture was cooled down to rt and triturated with 20 ml of DCM and filtered. The cake was rinsed with 10 ml of DCM and the filtrate was concentrated under reduced pressure. The residue was purified by flash chromatography on silica gel using a gradient of cyclohexane/EtOAc/MeOH (from 100/0/0 to 0/85/15) to give the title compound (414 mg, 30% yield) as a beige solid.

LCMS (Method V): Rt=0.50; MS m/z [M+H]⁺=267

### rac-(4aR,8aR)-1-[6-(2-hydroxy-4,6-dimethyl-phenyl)pyridazin-3-yl]-2,3,4,4a,5,6,7,8a-octahydro-1,7-naphthyridin-8-one (Example 300)

Under Ar, to a solution of (2-hydroxy-4,6-dimethyl-phenyl)boronic acid (1.5 mmol, 249 mg) and rac-(4aR,8aR)-1-(6-chloropyridazin-3-yl)-2,3,4,4a,5,6,7,8a-octahydro-1,7-naphthyridin-8-one (0.75 mmol, 200 mg) in 10 ml of 1,4-dioxane were added a 2N sodium carbonate aqueous solution (2.02 mmol, 1 ml) and tetrakis(triphenylphosphine)palladium(0) (0.037 mmol, 43 mg). The mixture was stirred at 100° C. for 1 h. After cooling down to rt, the mixture was diluted with 50 ml of EtOAc and 50 ml of diethylether and the organic layer was washed successively with 2×20 ml of a saturated aqueous sodium bicarbonate solution and 10 ml of water. The organic layer was dried over Na₂SO₄, filtered and concentrated under reduced pressure. The residue was purified by flash chromatography on silica gel eluting with a gradient of 0 to 10% of MeOH in DCM to give, after trituration in diethylether, the title compound as a yellow solid (43 mg, 16% yield).

### Example 290: rac-(4aR,8aR)-1-[6-(2-hydroxy-4,6-dimethyl-phenyl)pyridazin-3-yl]-7-methyl-3,4,4a,5,6,8a-hexahydro-2H-1,7-naphthyridin-8-one

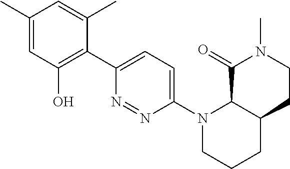 [SMILES: Cc1cc(C)c(-c2ccc(N3CCC[C@@H]4CCN(C)C(=O)[C@@H]43)nn2)c(O)c1]

### Int 290.1: rac-(4aR,8aR)-1-(6-chloropyridazin-3-yl)-7-methyl-3,4,4a,5,6,8a-hexahydro-2H-1,7-naphthyridin-8-one

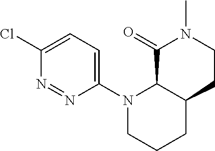 [SMILES: CN1CC[C@H]2CCCN(c3ccc(Cl)nn3)[C@H]2C1=O]

Under argon, at 0° C., to a solution of rac-(4aR,8aR)-4-(6-chloropyridazin-3-yl)-3,4a,6,7,8,8a-hexahydro-2H-pyrido[4,3-b][1,4]oxazin-5-one (Int 300.1-0.80 mmol, 214 mg) in 6 ml of anhydrous N,N-dimethylformamide was added a 60% oil dispersion of sodium hydride (1.2 mmol, 48 mg) and the mixture was stirred at rt for 2 h. Iodomethane (0.88 mmol, 55 μl) was added and the mixture was stirred for 2 h at rt then diluted with 50 ml of EtOAc, 50 ml of diethylether and 50 ml of water. The organic layer was washed with 2×30 ml of water, dried over Na₂SO₄, filtered and concentrated under reduced pressure. The residue was purified by flash chromatography on silica gel eluting with a gradient of 0 to 10% of MeOH in DCM to give the title compound as a yellow oil (225 mg, 100% yield).

LCMS (Method V): Rt=0.57; MS m/z [M+H]⁺=281

### rac-(4aR,8aR)-1-[6-(2-hydroxy-4,6-dimethyl-phenyl)pyridazin-3-yl]-7-methyl-3,4,4a,5,6,8a-hexahydro-2H-1,7-naphthyridin-8-one (Example 290)

Following procedure of Example 300, rac-(4aR,8aR)-1-(6-chloropyridazin-3-yl)-7-methyl-3,4,4a,5,6,8a-hexahydro-2H-1,7-naphthyridin-8-one (0.8 mmol, 225 mg) gave the title compound as a white solid (59 mg, 20% yield).

### Example 355: 2-[6-[(4aS,8aR)-6-(2,2,2-trifluoroethyl)-3,4a,5,7,8,8a-hexahydro-2H-pyrido[4,3-b][1,4]oxazin-4-yl]pyridazin-3-yl]-3,5-dimethyl-phenol

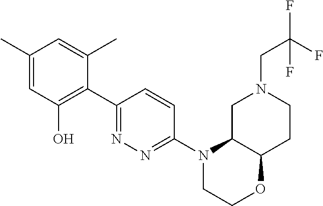 [SMILES: Cc1cc(C)c(-c2ccc(N3CCO[C@@H]4CCN(CC(F)(F)F)C[C@@H]43)nn2)c(O)c1]

### Int 355.1: (4aS,8aR)-4-(6-chloropyridazin-3-yl)-6-(2,2,2-trifluoroethyl)-3,4a,5,7,8,8a-hexahydro-2H-pyrido[4,3-b][1,4]oxazine

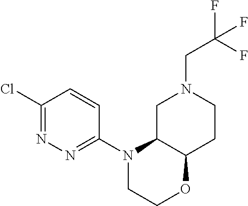 [SMILES: FC(F)(F)CN1CC[C@H]2OCCN(c3ccc(Cl)nn3)[C@H]2C1]

Under argon, at 0° C., to a solution of 1-[(4aS,8aR)-4-(6-chloropyridazin-3-yl)-3,4a,5,7,8,8a-hexahydro-2H-pyrido[4,3-b][1,4]oxazin-6-yl]-2,2,2-trifluoro-ethanone (Int 347.1-1.43 mmol, 500 mg) in 20 ml of anhydrous THF was added dropwise a 1N borane tetrahydrofurane complex THF solution (2.85 mmol, 2.85 ml). The mixture was stirred for 4 h at reflux then cooled down to rt. 1.43 ml (1.43 mmol) of 1N borane tetrahydrofurane complex THF solution was then added, and the mixture was stirred for another 4 h at reflux, cooled down to rt and quenched with 10 ml of a 4N HCl dioxane solution under stirring for 1 h. The mixture was then diluted with 100 ml of diethylether and 200 ml of a saturated aqueous sodium bicarbonate solution. The aqueous layer was extracted with 2×100 ml of diethylether and the combined organic layers were dried over Na₂SO₄, filtered and concentrated under reduced pressure. The residue was purified by flash chromatography on silica gel eluting with a gradient of 0 to 5% of MeOH in DCM to give the title compound as a yellow oil (187 mg, 38% yield).

LCMS (Method V): Rt=0.97; MS m/z [M+H]⁺=337

### 2-[6-[(4aS,8aR)-6-(2,2,2-trifluoroethyl)-3,4a,5,7,8,8a-hexahydro-2H-pyrido[4,3-b][1,4]oxazin-4-yl]pyridazin-3-yl]-3,5-dimethyl-phenol (Example 355)

Under Ar, to a solution of (2-hydroxy-4,6-dimethyl-phenyl)boronic acid (0.83 mmol, 138 mg) and (4aS,8aR)-4-(6-chloropyridazin-3-yl)-6-(2,2,2-trifluoroethyl)-3,4a,5,7,8,8a-hexahydro-2H-pyrido[4,3-b][1,4]oxazine (0.56 mmol, 187 mg) in 8 ml of 1,4-dioxane were added a 2N sodium carbonate aqueous solution (1.5 mmol, 750 μl) and tetrakis(triphenylphosphine)palladium(0) (0.028 mmol, 32 mg). The mixture was stirred at 100° C. for 1 h. After cooling down to rt, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered and concentrated under reduced pressure. The residue was purified by flash chromatography on silica gel using a gradient of DCM/MeOH/ACN (from 100/0/0 to 85/7.5/7.5) then repurified by prep-HPLC (column: Sunfire prep C18 OBD; 250*50 mm*10 μm; mobile phase: [aqueous TFA 10⁻² N/ACN] from 10% to 100% ACN in 35 min. at 50 ml/min) to give the title compound as a white solid (18 mg, 8% yield).

### Example 393 and Example 407: 2-[6-[(3aR,7aS)-2,3,3a,4,5,6,7,7a-octahydropyrrolo[2,3-c]pyridin-1-yl]pyridazin-3-yl]-3-methyl-5-(trifluoromethyl)phenol and 2-[6-[(3aS,7aR)-2,3,3a,4,5,6,7,7a-octahydropyrrolo[2,3-c]pyridin-1-yl]pyridazin-3-yl]-3-methyl-5-(trifluoromethyl)phenol

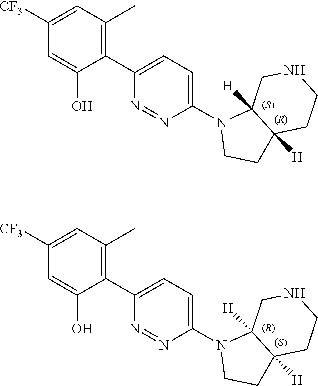

### Int 393.1: tert-butyl (3aR,7aR)-1-(6-chloropyridazin-3-yl)-3,3a,4,5,7,7a-hexahydro-2H-pyrrolo[2,3-c]pyridine-6-carboxylate

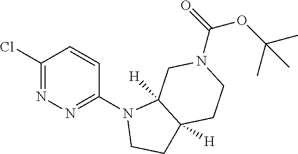 [SMILES: CC(C)(C)OC(=O)N1CC[C@H]2CCN(c3ccc(Cl)nn3)[C@H]2C1]

In a microwave vial, a mixture tert-butyl 1,2,3,3a,4,5,7,7a-octahydropyrrolo[2,3-c]pyridine-6-carboxylate (900 mg, 3.98 mmol), 3,6-dichloropyridazine (592 mg, 3.98 m mol) and K₃PO₄ (1.69 g, 7.95 mmol) in 13 ml of DMSO was heated at 80° C. for 2 h under microwave irradiations. After cooling down to rt, the mixture was filtered and concentrated under reduced pressure. The residue was purified by flash chromatography on silica gel eluting with a gradient of 0 to 5% of MeOH in DCM to give the title compound (776 mg, 57% yield).

LCMS (Method S): Rt=1.22; MS m/z [M+H]+=339

### Int 393.2: tert-butyl (3aR,7aR)-1-[6-[2-hydroxy-6-methyl-4-(trifluoromethyl)phenyl]pyridazin-3-yl]-3,3a,4,5,7,7a-hexahydro-2H-pyrrolo[2,3-c]pyridine-6-carboxylate

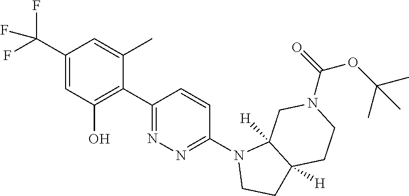 [SMILES: Cc1cc(C(F)(F)F)cc(O)c1-c1ccc(N2CC[C@@H]3CCN(C(=O)OC(C)(C)C)C[C@@H]32)nn1]

In a sealed tube, a mixture of tert-butyl 1-(6-chloropyridazin-3-yl)-3,3a,4,5,7,7a-hexahydro-2H-pyrrolo[2,3-c]pyridine-6-carboxylate (100 mg, 0.295 mmol), K₂CO₃ (81.6 mg, 0.590 mmol) and 3-methyl-2-(4,4,5,5-tetramethyl-1,3,2-dioxaborolan-2-yl)-5-(trifluoromethyl) phenol (0.134 g, 0.000443 mol) in 2 ml of 1,4-dioxane and 0.5 ml of water was purged with N₂. XPhos Pd G2 (11.6 mg, 0.0148 mmol) and XPhos (7.03 mg, 0.0148 mmol) were added, the mixture was purged again with N₂ then heated at 90° C. for 3 h. After cooling down to rt, the reaction mixture was concentrated under reduced pressure. The residue was purified by flash chromatography on silica gel eluting with a gradient of 0 to 5% of MeOH in DCM to give the title compound (90.8 mg, 64% yield).

LCMS (Method R): Rt=1.50; MS m/z [M+H]+=479.5

### Int 393.3: 2-[6-[rac-(3aS,7aR)-2,3,3a,4,5,6,7,7a-octahydropyrrolo[2,3-c]pyridin-1-yl]pyridazin-3-yl]-3-methyl-5-(trifluoromethyl)phenol

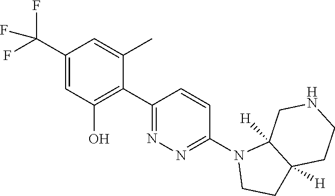 [SMILES: Cc1cc(C(F)(F)F)cc(O)c1-c1ccc(N2CC[C@@H]3CCNC[C@@H]32)nn1]

In around bottom flask, a stirred solution of tert-butyl (3aR,7aR)-1-[6-[2-hydroxy-6-methyl-4-(trifluoromethyl)phenyl]pyridazin-3-yl]-3,3a,4,5,7,7a-hexahydro-2H-pyrrolo[2,3-c]pyridine-6-carboxylate (81.0 mg, 0.169 mmol) in 2 ml of 1,4-dioxane was cooled down to 0° C. Then HCl (4 M in dioxane) (4.00 mol/L, 0.212 mL, 0.846 mmol) was added dropwise to the mixture. The mixture was stirred at rt overnight. The reaction mixture was concentrated under reduced pressure. The residue was triturated in a mixture of DIPE/ACN. The precipitate was filtered off and dried under reduced pressure. This residue was solubilized in MeOH, then concentrated under reduced pressure before being triturated in diethyl ether. The precipitate was filtered off and dried under reduced pressure. This precipitate was purified by reverse phase preparative LC (Column YMC—Actus Triart Prep C18-S 150*30 mm 5 μm Flow rate 50 ml/min-Focused gradient MeCN/aq·NH₄HCO₃ 0.2% pH=7.9 Focused gradient from 50/50 to 70/30) to afford a white solid. The solid was then purified by preparative achiral SFC (Stationary phase: Viridis 2-EP 20×250 mm, Mobile phase: CO₂/(MeOH+0.3% iPrNH₂) 85/15) to give 32 mg of a greyish oil (46%). The oil was triturated in DIPE. The precipitate was filtered and dried under reduced pressure to give a grey solid. This solid was purified by normal phase preparative LC (regular SiOH, 50 μm, 4 g biotage, dry loading (Celite®), mobile phase gradient: from DCM/(MeOH/NH₄OH (10%)) 100/0 to 75/25 over 20 CV) to give the title compound as a white solid (23.9 mg, 34%).

LCMS (Method E): Rt=2.47; [M+H]+=379.5

### 2-[6-[(3aR,7aS)-2,3,3a,4,5,6,7,7a-octahydropyrrolo[2,3-c]pyridin-1-yl]pyridazin-3-yl]-3-methyl-5-(trifluoromethyl)phenol and 2-[6-[(3aS,7aR)-2,3,3a,4,5,6,7,7a-octahydropyrrolo[2,3-c]pyridin-1-yl]pyridazin-3-yl]-3-methyl-5-(trifluoromethyl) phenol (Example 393 and Example 407)

2-[6-[rac-(3aS,7aR)-2,3,3a,4,5,6,7,7a-octahydropyrrolo[2,3-c]pyridin-1-yl]pyridazin-3-yl]-3-methyl-5-(trifluoromethyl)phenol (23.9 mg, 0.0576 mmol) was purified by preparative chiral SFC (Stationary phase: Chiralpak® IC 20×250 mm, Mobile phase: CO₂/(MeOH+0.3% iPrNH₂) 50/50). The fractions containing each enantiomer were separately combined and concentrated under reduced pressure to give a first fraction (14 mg, 22%) as a colorless oil and a second fraction (10 mg, 16%) as a colourless oil. The first fraction was taken-up with MeCN (1 mL), extended with water (3 mL) then lyophilized overnight to give 14 mg (22%) of a first enantiomer as a white solid. The second fraction was purified by reverse phase preparative LC (Column YMC—Actus Triart Prep C18-S 150*30 mm 5 μm Flow rate 50 ml/min-Focused gradient MeCN/aq·NH₄HCO₃ 0.2% pH=7.9 Focused gradient from 25/75 to 65/35) to give, after lyophilization, 6 mg (9%) of a second enantiomer as a white solid. The absolute configurations cannot be assigned at this stage.

### Example 394: 1-[rac-(3aR,7aR)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethanone

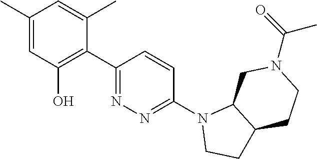 [SMILES: CC(=O)N1CC[C@H]2CCN(c3ccc(-c4c(C)cc(C)cc4O)nn3)[C@H]2C1]

### Int 394.1: tert-butyl rac-(3aR,7aR)-1-(6-chloropyridazin-3-yl)-3,3a,4,5,7,7a-hexahydro-2H-pyrrolo[2,3-c]pyridine-6-carboxylate

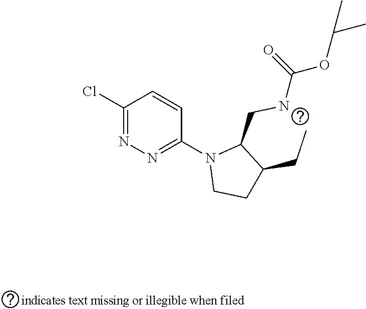 [SMILES: CC[C@H]1CCN(c2ccc(Cl)nn2)[C@H]1CNC(=O)OC(C)C]

Under Ar, a solution of tert-butyl 1,2,3,3a,4,5,7,7a-octahydropyrrolo[2,3-c]pyridine-6-carboxylate (19 mmol, 4.2 g—as a mixture of cis and trans racemates), 3,6-dichloropyridazine (28 mmol, 4.1 g) and N,N-diisopropylethylamine (74 mmol, 13 ml) in 30 ml of 1-butanol was stirred at reflux for 8 h. The mixture was cooled down to rt, then diluted with 100 ml of EtOAc and 100 ml of diethylether and filtered. The cake was rinsed with 50 ml of diethylether and the combined filtrates were concentrated under reduced pressure. The residue was purified by flash chromatography on silica gel eluting with a gradient of 0 to 100% of EtOAc in cyclohexane to give the title compound (2.77 g, 43% yield) as a white foam.

LCMS (Method V): Rt=0.77; MS m/z [M+H]⁺=339

### Int 394.2: rac-(3aS,7aR)-1-(6-chloropyridazin-3-yl)-2,3,3a,4,5,6,7,7a-octahydropyrrolo [2,3-c]pyridine; dihydrochloride

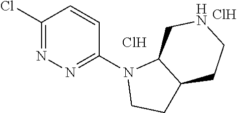 [SMILES: Clc1ccc(N2CC[C@@H]3CCNC[C@@H]32)nn1 ; Cl ; Cl]

To a solution of tert-butyl rac-(3aR,7aR)-1-(6-chloropyridazin-3-yl)-3,3a,4,5,7,7a-hexahydro-2H-pyrrolo[2,3-c]pyridine-6-carboxylate (5.9 mmol, 2 g) in 20 ml of dichloromethane and 10 ml of MeOH was added 14.8 ml of a 4N hydrochloric acid solution in 1,4-dioxane and the mixture was stirred at rt for 18 h then concentrated under reduced pressure. The residue was triturated in 50 ml of diethylether, filtered off, and the cake rinsed with 50 ml of diethylether, dried under reduced pressure to give the title compound (1.86 g, 100% yield) as a light brown solid.

LCMS (Method L): Rt=0.39; MS m/z [M+H]⁺=239

### Int 394.3: 1-[rac-(3aR,7aR)-1-(6-chloropyridazin-3-yl)-3,3a,4,5,7,7a-hexahydro-2H-pyrrolo[2,3-c]pyridin-6-yl]ethanone

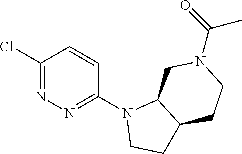 [SMILES: CC(=O)N1CC[C@H]2CCN(c3ccc(Cl)nn3)[C@H]2C1]

Under Ar, to a suspension of rac-(3aS,7aR)-1-(6-chloropyridazin-3-yl)-2,3,3a,4,5,6,7,7a-octahydropyrrolo[2,3-c]pyridine: dihydrochloride (2.89 mmol, 903 mg) in 30 ml of DCM were added DIPEA (8.96 mmol, 1.24 ml), acetic anhydride (4.34 mmol, 907 μl) and DMAP (0.14 mmol, 18 mg). The mixture was stirred at rt for 18 h then diluted with 100 ml of dichloromethane and washed with 100 ml of a saturated aqueous sodium bicarbonate solution. The organic layer was dried over Na₂SO₄, filtered and concentrated under reduced pressure to give the title compound (680 mg, 84% yield) as a yellow solid.

LCMS (Method V): Rt=0.64; MS m/z [M+H]⁺=281

### 1-[rac-(3aR,7aR)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethenone (Example 394)

Under Ar, to a solution of (2-hydroxy-4,6-dimethyl-phenyl)boronic acid (2.42 mmol, 402 mg) and 1-[rac-(3aR,7aR)-1-(6-chloropyridazin-3-yl)-3,3a,4,5,7,7a-hexahydro-2H-pyrrolo[2,3-c] pyridin-6-yl]ethanone (1.21 mmol, 340 mg) in 15 ml of 1,4-dioxane was added a 2N sodium carbonate aqueous solution (3.27 mmol, 1.63 ml) then tetrakis(triphenylphosphine) palladium(0) (0.061 mmol, 70 mg). The mixture was stirred at 100° C. for 2 h. After cooling down, the mixture was diluted with 100 ml of EtOAc and 100 ml of diethylether. The organic layer was washed successively with 2×100 ml of a saturated aqueous sodium bicarbonate solution and 105 ml of water. The organic layer was dried over Na₂SO₄, filtered and concentrated under reduced pressure. The residue was purified by flash chromatography on silica gel using a gradient of DCM/MeOH/ACN (from 100/0/0 to 85/7.5/7.5) to give the title compound as a yellow solid (85 mg, 19% yield).

Compound Example 395 was synthetized analogously to Example 394

### Example 413 and 414: 1-[(3aR,7aR)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethenone and 1-[(3aS,7aS)-1-[6-(2-hydroxy-4,6-dimethyl-phenyl)pyridazin-3-yl]-3,3a,4,5,7,7a-hexahydro-2H-pyrrolo [2,3-c] pyridin-6-yl]ethanone

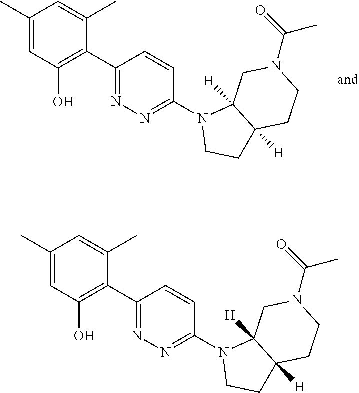

Both enantiomers of 1-[rac-(3aR,7aR)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethanone (Example 394-71 mg) were separated by Supercritical Fluidic Chromatography (SFC—Column: Daicel Chiralpak® AD, 300×50 mm I.D., 20 μm particle size; Mobile phase: CO₂/Methanol (+0.1% Triethylamine) 40%; flow rate: 220 mL/min; outlet pressure: 100 bar; 3 injections; UV detection: 265 nm) to provide 1-[(3aR,7aR)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethanone, first enantiomer eluting at Rt=7.47 min (27 mg, Example 413, Enantiomer 1), as a white solid, and 1-[(3aS,7aS)-1-[6-(2-hydroxy-4,6-dimethyl-phenyl)pyridazin-3-yl]-3,3a,4,5,7,7a-hexahydro-2H-pyrrolo[2,3-c]pyridin-6-yl] ethanone, second enantiomer eluting at Rt=12.06 min (26 mg, Example 414, Enantiomer 2), as a white solid. The absolute configurations cannot be assigned at this stage.

Compounds Examples 369 and 370 were separated analogously to Examples 413 and 414 using a chiral column Daicel Chiralpak® AS.

### Example 342: 3,5-dimethyl-2-[6-[rac-(3aS,7aR)-6-methyl-3,3a,4,5,7,7a-hexahydro-2H-pyrrolo[2,3-c]pyridin-1-yl]pyridazin-3-yl]phenol

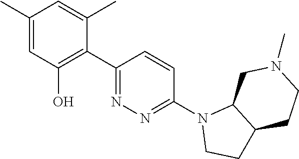 [SMILES: Cc1cc(C)c(-c2ccc(N3CC[C@@H]4CCN(C)C[C@@H]43)nn2)c(O)c1]

### Int 342.1: rac-(3aS,7aR)-1-(6-chloropyridazin-3-yl)-6-methyl-3,3a,4,5,7,7a-hexahydro-2H-pyrrolo[2,3-c]pyridine

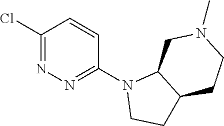 [SMILES: CN1CC[C@H]2CCN(c3ccc(Cl)nn3)[C@H]2C1]

To a suspension of rac-(3aS,7aR)-1-(6-chloropyridazin-3-yl)-2,3,3a,4,5,6,7,7a-octahydropyrrolo[2,3-c]pyridine; dihydrochloride (Int 394.2-1.93 mmol, 600 mg) in 20 ml of dichloromethane were added successively triethylamine (4.81 mmol, 691 μl), a 37% weight formaldehyde aqueous solution (4.81 mmol, 376 μl) and sodium triacetoxyborohydride (7.7 mmol, 1.63 g) portion wise. The mixture was stirred at rt for 8 h then diluted with 100 ml of dichloromethane and 100 ml of a saturated aqueous sodium bicarbonate solution. The organic layer was dried over Na₂SO₄, filtered and concentrated under reduced pressure. The residue was purified by flash chromatography on silica gel eluting with a gradient of 0 to 10% of MeOH in DCM to give the title compound as a beige solid (449 mg, 92% yield).

LCMS (Method L): Rt=0.42; MS m/z [M+H]⁺=253

### 3,5-dimethyl-2-[6-[rac-(3aS,7aR)-6-methyl-3,3a,4,5,7,7a-hexahydro-2H-pyrrolo[2,3-c]pyridin-1-yl]pyridazin-3-yl]phenol (Example 342)

Under Ar, to a solution of (2-hydroxy-4,6-dimethyl-phenyl)boronic acid (0.87 mmol, 144 mg) and rac-(3aS,7aR)-1-(6-chloropyridazin-3-yl)-6-methyl-3,3a,4,5,7,7a-hexahydro-2H-pyrrolo[2,3-c]pyridine (0.44 mmol, 110 mg) in 4 ml of 1,4-dioxane and 800 μl of water, was added cesium carbonate (1.31 mmol, 425 mg). The mixture was degased by argon bubbling for 5 min then [1,1′-bis(diphenylphosphino)ferrocene]dichloropalladium(II) (0) (0.044 mmol, 32 mg) was added and the mixture was stirred at 90° C. for 4 h. After cooling down, the mixture was diluted with 50 ml of DCM and the organic layer was washed with 50 ml of a saturated aqueous sodium bicarbonate solution. The aqueous layer was extracted with 20 ml of DCM and the combined organic layers were dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 80/9.8/9.8/0.4) to give the title compound as a brown solid (100 mg, 68% yield).

Compound Examples 364, 371 and 378 were synthesized analogously to Example 342

### Example 383 and 384: 2-[6-[(3aR,7aS)-6-methyl-3,3a,4,5,7,7a-hexahydro-2H-pyrrolo[2,3-c]pyridin-1-yl]pyridazin-3-yl]-3,5-dimethyl-phenol and 2-[6-[(3aS,7aR)-6-methyl-3,3a,4,5,7,7a-hexahydro-2H-pyrrolo[2,3-c]pyridin-1-yl]pyridazin-3-yl]-3,5-dimethyl-phenol

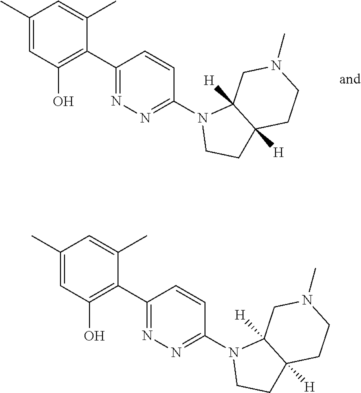 [SMILES: Cc1cc(C)c(-c2ccc(N3CC[C@H]4CCN(C)C[C@H]43)nn2)c(O)c1 ; Cc1cc(C)c(-c2ccc(N3CC[C@@H]4CCN(C)C[C@@H]43)nn2)c(O)c1]

Both enantiomers of 3,5-dimethyl-2-[6-[rac-(3aS,7aR)-6-methyl-3,3a,4,5,7,7a-hexahydro-2H-pyrrolo[2,3-c]pyridin-1-yl]pyridazin-3-yl]phenol (Example 342-160 mg) were separated by chiral preparative HPLC (Column: Daicel Chiralpak® AY, 230×100 mm I.D., 20 μm particle size; Mobile phase: Heptane/Ethanol (+0.1% Triethylamine) 20%; flow rate: 400 mL/min; 1 injection; UV detection: 265 nm) to provide 2-[6-[(3aR,7aS)-6-methyl-3,3a,4,5,7,7a-hexahydro-2H-pyrrolo[2,3-c]pyridin-1-yl]pyridazin-3-yl]-3,5-dimethyl-phenol, first enantiomer eluting at Rt=12.9 min (62 mg, Example 383, Enantiomer 1), as a white solid, and 2-[6-[(3aS,7aR)-6-methyl-3,3a,4,5,7,7a-hexahydro-2H-pyrrolo[2,3-c]pyridin-1-yl]pyridazin-3-yl]-3,5-dimethyl-phenol, second enantiomer eluting at Rt=18.8 min (61 mg, Example 384, Enantiomer 2), as a white solid. The absolute configurations cannot be assigned at this stage.

Compounds Examples 391; 392 and 406; 429 were synthesized separated to Example 383/384 using a chiral column Daicel Chiralpak® AD.

### Example 385: 3-methyl-2-[6-[rac-(3aS,7aR)-6-methyl-3,3a,4,5,7,7a-hexahydro-2H-pyrrolo[2,3-c]pyridin-1-yl]pyridazin-3-yl]phenol

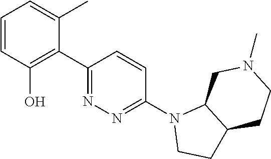 [SMILES: Cc1cccc(O)c1-c1ccc(N2CC[C@@H]3CCN(C)C[C@@H]32)nn1]

Under Ar, to a solution of (2-hydroxy-6-methyl-phenyl)boronic acid (Int 354.1-0.65 mmol, 98 mg) and rac-(3aS,7aR)-1-(6-chloropyridazin-3-yl)-6-methyl-3,3a,4,5,7,7a-hexahydro-2H-pyrrolo[2,3-c]pyridine (Int 342.1-0.40 mmol, 102 mg) in 4 ml of 1,4-dioxane were added a 2N sodium carbonate aqueous solution (1.09 mmol, 545 μl) and TETRAKIS(triphenylphosphine)palladium(0) (0.02 mmol, 23 mg). The mixture was stirred at 100° C. for 3 h30 then cooled down to rt and diluted with 50 ml of EtOAc. The organic layer was washed with 20 ml of water and the aqueous layer extracted with 20 ml of EtOAc. The combined organic layers were washed with 10 ml of brine, dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 90/4.9/4.9/0.2) to give the title compound as a white solid (70 mg, 53% yield).

### Examples 415 and 416: 2-[6-[(3aS,7aR)-6-methyl-3,3a,4,5,7,7a-hexahydro-2H-pyrrolo[2,3-c]pyridin-1-yl]pyridazin-3-yl]-3-methyl-phenol and 2-[6-[(3aR,7aS)-6-methyl-3,3a,4,5,7,7a-hexahydro-2H-pyrrolo[2,3-c]pyridin-1-yl]pyridazin-3-yl]-3-methyl-phenol

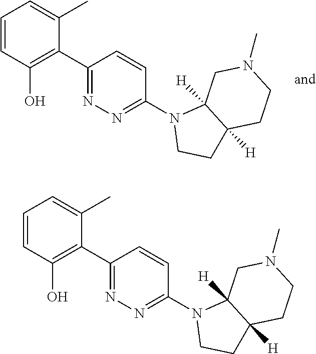 [SMILES: Cc1cccc(O)c1-c1ccc(N2CC[C@@H]3CCN(C)C[C@@H]32)nn1 ; Cc1cccc(O)c1-c1ccc(N2CC[C@H]3CCN(C)C[C@H]32)nn1]

Both enantiomers of 3-methyl-2-[6-[rac-(3aS,7aR)-6-methyl-3,3a,4,5,7,7a-hexahydro-2H-pyrrolo[2,3-c]pyridin-1-yl]pyridazin-3-yl]phenol (Example 385-65 mg) were separated by Supercritical Fluidic Chromatography (SFC—Column: Daicel Chiralpak® AY-H, 350×30 mm I.D., 5 μm particle size; Mobile phase: CO₂/Ethanol (+0.1% Triethylamine) 40%; flow rate: 120 mL/min; outlet pressure: 100 bar; 3 injections; UV detection: 265 nm) to provide 2-[6-[(3aS,7aR)-6-methyl-3,3a,4,5,7,7a-hexahydro-2H-pyrrolo[2,3-c]pyridin-1-yl]pyridazin-3-yl]-3-methyl-phenol, first enantiomer eluting at Rt=3.17 min (23 mg, Example 415, Enantiomer 1), as a white solid, and 2-[6-[(3aR,7aS)-6-methyl-3,3a,4,5,7,7a-hexahydro-2H-pyrrolo[2,3-c]pyridin-1-yl]pyridazin-3-yl]-3-methyl-phenol, second enantiomer eluting at Rt=4.79 min (22 mg, Example 415, Enantiomer 2), as a white solid. The absolute configurations cannot be assigned at this stage.

### Example 388: 3-ethyl-2-[6-[rac-(3aS,7aR)-6-methyl-3,3a,4,5,7,7a-hexahydro-2H-pyrrolo[2,3-c]pyridin-1-yl]pyridazin-3-yl]phenol

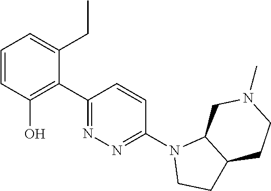 [SMILES: CCc1cccc(O)c1-c1ccc(N2CC[C@@H]3CCN(C)C[C@@H]32)nn1]

Under Ar, to a solution of (2-ethyl-6-hydroxy-phenyl)boronic acid (Int 358.2-0.66 mmol, 109 mg) and rac-(3aS,7aR)-1-(6-chloropyridazin-3-yl)-6-methyl-3,3a,4,5,7,7a-hexahydro-2H-pyrrolo[2,3-c]pyridine (Int 342.1-0.41 mmol, 104 mg) in 4 ml of 1,4-dioxane were added a 2N sodium carbonate aqueous solution (1.11 mmol, 555 μl) and tetrakis(triphenylphosphine)palladium(0) (0.021 mmol, 24 mg). The mixture was stirred at 100° C. for 3 h30 then cooled down to rt and diluted with 50 ml of EtOAc. The organic layer was washed with 20 ml of water and the aqueous layer extracted with 20 ml of EtOAc. The combined organic layers were washed with 10 ml of brine, dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 90/4.9/4.9/0.2) to give the title compound as a white solid (81 mg, 58% yield).

### Examples 422 and 423: 2-[6-[(3aS,7aR)-6-methyl-3,3a,4,5,7,7a-hexahydro-2H-pyrrolo[2,3-c]pyridin-1-yl]pyridazin-3-yl]-3-ethyl-phenol and 2-[6-[(3aR,7aS)-6-methyl-3,3a,4,5,7,7a-hexahydro-2H-pyrrolo[2,3-c]pyridin-1-yl]pyridazin-3-yl]-3-ethyl-phenol

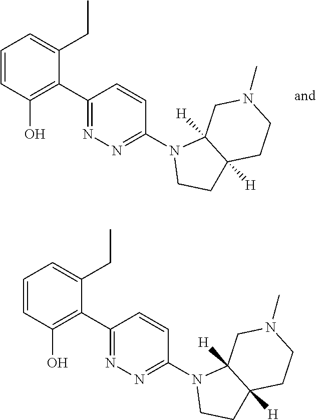 [SMILES: CCc1cccc(O)c1-c1ccc(N2CC[C@@H]3CCN(C)C[C@@H]32)nn1 ; CCc1cccc(O)c1-c1ccc(N2CC[C@H]3CCN(C)C[C@H]32)nn1]

Both enantiomers of 3-ethyl-2-[6-[rac-(3aS,7aR)-6-methyl-3,3a,4,5,7,7a-hexahydro-2H-pyrrolo[2,3-c]pyridin-1-yl]pyridazin-3-yl]phenol (Example 388-75 mg)) were separated by Supercritical Fluidic Chromatography (SFC—Column: Daicel Chiralpak® AY-H, 350×30 mm I.D., 5 μm particle size; Mobile phase: CO₂/Ethanol (+0.1% Triethylamine) 40%; flow rate: 120 mL/min; outlet pressure: 100 bar; 3 injections; UV detection: 265 nm) to provide 2-[6-[(3aS,7aR)-6-methyl-3,3a,4,5,7,7a-hexahydro-2H-pyrrolo[2,3-c]pyridin-1-yl]pyridazin-3-yl]-3-ethyl-phenol, first enantiomer eluting at Rt=2.64 min (29 mg, Example 422, Enantiomer 1), as a white solid, and 2-[6-[(3aR,7aS)-6-methyl-3,3a,4,5,7,7a-hexahydro-2H-pyrrolo[2,3-c]pyridin-1l-yl]pyridazin-3-yl]-3-ethyl-phenol, second enantiomer eluting at Rt=4.50 min (31 mg, Example 423, Enantiomer 2), as a white solid. The absolute configurations cannot be assigned at this stage.

### Example 389: 2-[6-[rac-(3aS,7aR)-6-methyl-3,3a,4,5,7,7a-hexahydro-2H-pyrrolo[2,3-c]pyridin-1l-yl]pyridazin-3-yl]-3-(trifluoromethyl)phenol

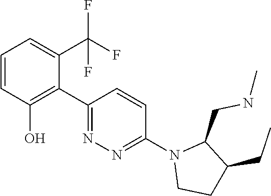 [SMILES: CC[C@H]1CCN(c2ccc(-c3c(O)cccc3C(F)(F)F)nn2)[C@H]1CNC]

Under Ar, to a solution of [2-hydroxy-6-(trifluoromethyl)phenyl]boronic acid (0.63 mmol, 130 mg) and rac-(3aS,7aR)-1-(6-chloropyridazin-3-yl)-6-methyl-3,3a,4,5,7,7a-hexahydro-2H-pyrrolo[2,3-c]pyridine (Int 342.1-0.40 mmol, 100 mg) in 4 ml of 1,4-dioxane were added a 2N sodium carbonate aqueous solution (1.07 mmol, 534 μl) and tetrakis(triphenylphosphine)palladium(0) (0.02 mmol, 23 mg). The mixture was stirred at 100° C. for 3 h then cooled down to rt and diluted with 50 ml of EtOAc. The organic layer was washed with 20 ml of water and the aqueous layer extracted with 20 ml of EtOAc. The combined organic layers were washed with 10 ml of brine, dried over Na₂S₄, filtered and concentrated under reduced pressure. The residue was purified by ash chromatography on silica gel using a gradient of DCM/MeOH/ACN/NH₃ 7N sol. in MeOH (from 100/0/0/0 to 90/4.9/4.9/0.2) to give the title compound as a white solid (119 mg, 79% yield).

### Example 426 and 427: 2-[6-[(3aR,7aS)-6-methyl-3,3a,4,5,7,7a-hexahydro-2H-pyrrolo[2,3-c]pyridin-1-yl]pyridazin-3-yl]-3-(trifluoromethyl)phenol and 2-[6-[(3aS,7aR)-6-methyl-3,3a,4,5,7,7a-hexahydro-2H-pyrrolo[2,3-c]pyridin-1-yl]pyridazin-3-yl]-3-(trifluoromethyl) phenol

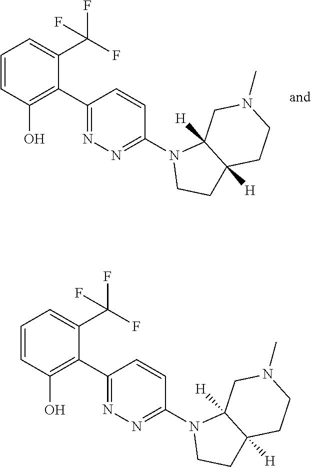 [SMILES: CN1CC[C@@H]2CCN(c3ccc(-c4c(O)cccc4C(F)(F)F)nn3)[C@@H]2C1 ; CN1CC[C@H]2CCN(c3ccc(-c4c(O)cccc4C(F)(F)F)nn3)[C@H]2C1]

Both enantiomers of 2-[6-[rac-(3aS,7aR)-6-methyl-3,3a,4,5,7,7a-hexahydro-2H-pyrrolo[2,3-c]pyridin-1-yl]pyridazin-3-yl]-3-(trifluoromethyl)phenol (Example 389-112 mg) were separated by chiral preparative HPLC (Column: LUX AMYLOSE-1, 250×30 mm I.D., 5 μm particle size; Mobile phase: Heptane/Ethanol (+0.1% Triethylamine) 30%; flow rate: 45 mL/min; 5 injection; UV detection: 265 nm) to provide 2-[6-[(3aR,7aS)-6-methyl-3,3a,4,5,7,7a-hexahydro-2H-pyrrolo[2,3-c]pyridin-1-yl]pyridazin-3-yl]-3-(trifluoromethyl) phenol, first enantiomer eluting at Rt=6.2 min (40 mg, Example 426, Enantiomer 1), as a white solid, and 2-[6-[(3aS,7aR)-6-methyl-3,3a,4,5,7,7a-hexahydro-2H-pyrrolo[2,3-c]pyridin-1-yl]pyridazin-3-yl]-3-(trifluoromethyl)phenol, second enantiomer eluting at Rt=12.3 min (38 mg, Example 427, Enantiomer 2), as a white solid. The absolute configurations cannot be assigned at this stage.

### Example 341: 5-methyl-2-[4-methyl-6-[rac-(3aS,7aR)-6-methyl-3,3a,4,5,7,7a-hexahydro-2H-pyrrolo[2,3-c]pyridin-1-yl]pyridazin-3-yl]phenol

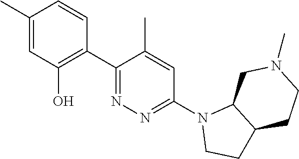 [SMILES: Cc1ccc(-c2nnc(N3CC[C@@H]4CCN(C)C[C@@H]43)cc2C)c(O)c1]

### Int 341.1: tert-butyl rac-(3aR,7aR)-1-(6-chloro-5-methyl-pyridazin-3-yl)-3,3a,4,5,7,7a-hexahydro-2H-pyrrolo[2,3-c]pyridine-6-carboxylate

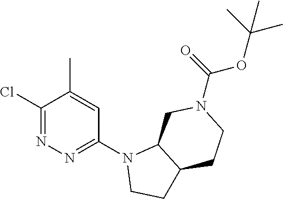 [SMILES: Cc1cc(N2CC[C@@H]3CCN(C(=O)OC(C)(C)C)C[C@@H]32)nnc1Cl]

Under Ar, a solution of tert-butyl 1,2,3,3a,4,5,7,7a-octahydropyrrolo[2,3-c]pyridine-6-carboxylate (12.27 mmol, 2.78 g—as a mixture of cis and trans racemates), 3,6-dichloro-4-methyl-pyridazine (12.27 mmol, 2 g) and N,N-diisopropylethylamine (49.1 mmol, 8.55 ml) in 20 ml of 1-butanol was stirred at reflux for 25 h. The mixture was cooled down to rt, concentrated under reduced pressure and the residue solubilized in 200 ml of EtOAc. The organic layer was washed with 2×50 ml of water then dried over Na₂SO₄, filtered and concentrated under reduced pressure. The residue was purified by flash chromatography on silica gel using a gradient of cyclohexane/EtOAc/MeOH (from 100/0/0 to 0/98/2) to give the title compound as a brown solid (1.14 g, 26% yield).

LCMS (Method L): Rt=0.76; MS m/z [M+H]⁺=353

### Int 341.2: rac-(3aS,7aR)-1-(6-chloro-5-methyl-pyridazin-3-yl)-2,3,3a,4,5,6,7,7a-octahydropyrrolo[2,3-c]pyridine; dihydrochloride

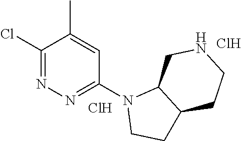 [SMILES: Cc1cc(N2CC[C@@H]3CCNC[C@@H]32)nnc1Cl ; Cl ; Cl]

Following procedure of Int 394.2, tert-butyl rac-(3aR,7aR)-1-(6-chloro-5-methyl-pyridazin-3-yl)-3,3a,4,5,7,7a-hexahydro-2H-pyrrolo[2,3-c]pyridine-6-carboxylate (3.22 mmol, 1.14 g) gave the title compound (1.05 g, 100% yield) as a colourless oil.

LCMS (Method L): Rt=0.40; MS m/z [M+H]⁺=253

### Int 341.3: rac-(3aS,7aR)-1-(6-chloro-5-methyl-pyridazin-3-yl)-6-methyl-3,3a,4,5,7,7a-hexahydro-2H-pyrrolo[2,3-c]pyridine

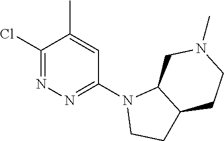 [SMILES: Cc1cc(N2CC[C@@H]3CCN(C)C[C@@H]32)nnc1Cl]

Following procedure of Int 342.1, rac-(3aS,7aR)-1-(6-chloro-5-methyl-pyridazin-3-yl)-2,3,3a,4,5,6,7,7a-octahydropyrrolo[2,3-c]pyridine; dihydrochloride (3.22 mmol, 1.05 g) gave the title compound (820 mg, 95% yield) as a colourless oil.

LCMS (Method V): Rt=0.65; MS m/z [M+H]⁺=267

### 5-methyl-2-[4-methyl-6-[rac-(3aS,7aR)-6-methyl-3,3a,4,5,7,7a-hexahydro-2H-pyrrolo [2,3-c]pyridin-1-yl]pyridazin-3-yl]phenol (Example 341)

Under Ar, to a solution of (2-hydroxy-4-methyl-phenyl)boronic acid (1.41 mmol, 214 mg) and rac-(3aS,7aR)-1-(6-chloro-5-methyl-pyridazin-3-yl)-6-methyl-3,3a,4,5,7,7a-hexahydro-2H-pyrrolo[2,3-c]pyridine (0.94 mmol, 250 mg) in 6 ml of 1,4-dioxane were added a 2N sodium carbonate aqueous solution (2.53 mmol, 1.27 ml) and Tetrakis(triphenylphosphine)palladium(0) (0.047 mmol, 54 mg). The mixture was stirred at 100° C. for 2 h. After cooling down to rt, the mixture was diluted with 100 ml of EtOAc and 100 ml of diethylether, and the organic layer was washed successively with 2×50 ml of a saturated aqueous sodium bicarbonate solution and 50 ml of water. The organic layer was dried over Na₂SO₄, filtered and concentrated under reduced pressure. The residue was purified by prep-HPLC (column: Sunfire prep C18 OBD; 250*50 mm*10 μm; mobile phase: [aqueous TFA 10⁻² N/ACN] from 10% to 100% ACN in 35 min. at 50 ml/min) to give the title compound as a white solid (203 mg, 64% yield).

### Example 374 and 375: 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-methyl-phenol and 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-methyl-phenol

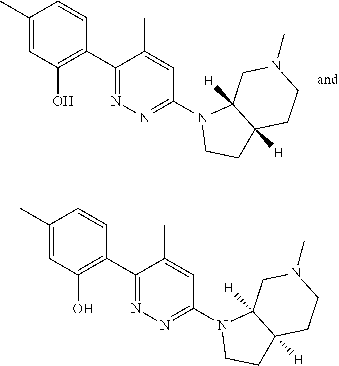 [SMILES: Cc1ccc(-c2nnc(N3CC[C@H]4CCN(C)C[C@H]43)cc2C)c(O)c1 ; Cc1ccc(-c2nnc(N3CC[C@@H]4CCN(C)C[C@@H]43)cc2C)c(O)c1]

Both enantiomers of 5-methyl-2-[4-methyl-6-[rac-(3aS,7aR)-6-methyl-3,3a,4,5,7,7a-hexahydro-2H-pyrrolo[2,3-c]pyridin-1-yl]pyridazin-3-yl]phenol (Example 341-193 mg) were separated by Supercritical Fluidic Chromatography (SFC—Column: Daicel Chiralpak® AY, 250×30 mm I.D., 5 μm particle size; Mobile phase: CO₂/Ethanol (+0.1% Triethylamine) 30%; flow rate: 120 mL/min; outlet pressure: 100 bar; 40 injections; UV detection: 265 nm) to provide 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-methyl-phenol, first enantiomer eluting at Rt=4.9 min (70 mg, Example 374, Enantiomer 1), as a white solid, and 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-methyl-phenol, second enantiomer eluting at Rt=6.7 min (77 mg, Example 375, Enantiomer 2), as a white solid. The absolute configurations cannot be assigned at this stage.

### Example 340: 2-[4-methyl-6-[rac-(3aS,7aR)-6-methyl-3,3a,4,5,7,7a-hexahydro-2H-pyrrolo[2,3-c]pyridin-1-yl]pyridazin-3-yl]-5-(trifluoromethyl)phenol

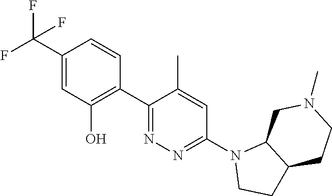 [SMILES: Cc1cc(N2CC[C@@H]3CCN(C)C[C@@H]32)nnc1-c1ccc(C(F)(F)F)cc1O]

Following procedure of Example 341, rac-(3aS,7aR)-1-(6-chloro-5-methyl-pyridazin-3-yl)-6-methyl-3,3a,4,5,7,7a-hexahydro-2H-pyrrolo[2,3-c]pyridine (Int 341.3-0.94 mmol, 250 mg) gave the title compound (101 mg, 27% yield) as a white solid.

### Example 366 and 367: 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-(trifluoromethyl)phenol and 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-(trifluoromethyl)phenol

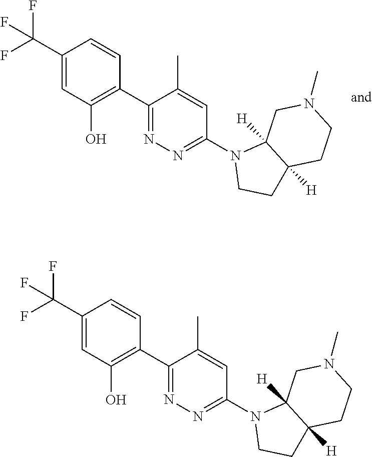 [SMILES: Cc1cc(N2CC[C@@H]3CCN(C)C[C@@H]32)nnc1-c1ccc(C(F)(F)F)cc1O ; Cc1cc(N2CC[C@H]3CCN(C)C[C@H]32)nnc1-c1ccc(C(F)(F)F)cc1O]

Both enantiomers of 2-[4-methyl-6-[rac-(3aS,7aR)-6-methyl-3,3a,4,5,7,7a-hexahydro-2H-pyrrolo[2,3-c]pyridin-1-yl]pyridazin-3-yl]-5-(trifluoromethyl)phenol (Example 340-86 mg) were separated by Supercritical Fluidic Chromatography (SFC—Column: Daicel Chiralpak® AD-H, 250×30 mm I.D., 5 μm particle size; Mobile phase: CO₂/Ethanol (+0.1% Triethylamine) 30%; flow rate: 120 mL/min; outlet pressure: 100 bar; 10 injections; UV detection: 265 nm) to provide 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-(trifluoromethyl)phenol, first enantiomer eluting at Rt=2.20 min (32 mg, Example 366, Enantiomer 1), as a white solid, and 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-(trifluoromethyl)phenol, second enantiomer eluting at Rt=2.88 min (36 mg, Example 367, Enantiomer 2), as a white solid. The absolute configurations cannot be assigned at this stage.

### Example 428: 3,5-dimethyl-2-[6-(9-methyl-1,9-diazaspiro[4.5]decan-1-yl)pyridazin-3-yl]phenol

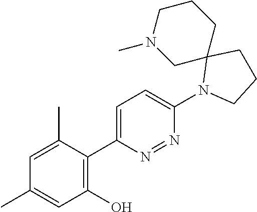 [SMILES: Cc1cc(C)c(-c2ccc(N3CCCC34CCCN(C)C4)nn2)c(O)c1]

### Int 428.1: tert-butyl 1-(6-chloropyridazin-3-yl)-1,9-diazaspiro[4.5]decane-9-carboxylate

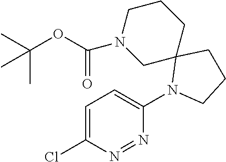 [SMILES: CC(C)(C)OC(=O)N1CCCC2(CCCN2c2ccc(Cl)nn2)C1]

In a microwave vial, under Ar, to a solution of 3,6-dichloropyridazine (1.530 mmol, 240 mg) in 10 ml of 1-butanol was added tert-butyl 1,7-diazaspiro[4.5]decane-7-carboxylate (1.530 mmol, 379 mg) and DIPEA (3.36 mmol, 0.592 mL). The vial was sealed, and the mixture was heated at 180° C. under microwave irradiation for 3 h. After cooling down to rt, the mixture was concentrated under reduced pressure to give a mixture of the title compound and 1-(6-chloropyridazin-3-yl)-1,9-diazaspiro[4.5]decane, as an oil (529 mg), which was used in the next step without further purification.

LCMS (Method H): Rt=1.09; MS m/z [M+H]+=353

### Int 428.2: 1-(6-chloropyridazin-3-yl)-1,9-diazaspiro[4.5]decane;hydrochloride

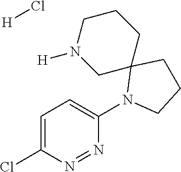 [SMILES: Cl ; Clc1ccc(N2CCCC23CCCNC3)nn1]

To a solution of tert-butyl 1-(6-chloropyridazin-3-yl)-1,9-diazaspiro[4.5]decane-9-carboxylate (1.53 mmol, 540 mg) in 3 ml of dichloromethane was added hydrochloric acid (4M/dioxane) (7.65 mmol, 1.91 mL). The mixture was stirred for 2 h30 at rt. The mixture was concentrated under reduced pressure, taken up in methanol (3×2 mL), and concentrated under reduced pressure to dryness to give the titled product as a solid (554 mg), which was used without further purification in the next step.

LCMS (Method H): Rt=0.29; MS m/z [M+H]+=253

### Int 428.3: 1-(6-chloropyridazin-3-yl)-9-methyl-1,9-diazaspiro[4.5]decane

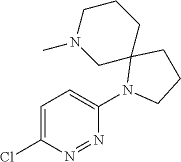 [SMILES: CN1CCCC2(CCCN2c2ccc(Cl)nn2)C1]

To a solution of 1-(6-chloropyridazin-3-yl)-1,9-diazaspiro[4.5]decane (0; 474 mmol, 120 mg) in 2 ml of methanol was added acetic acid (1.9 mmol, 0.113 mL) and formaldehyde 37 wt. % in water (0.588 mmol, 0.055 mL). The mixture was stirred for 20 min at rt. Sodium cyanoborohydride (0.946 mmol, 74.6 mg) was added and the mixture was stirred for 4 h at rt. Dichloromethane was added, then NaOH 1N until pH 10. The aqueous layer was extracted with AcOEt (3 times), and the combined organic layers were dried over MgSO₄, filtered and concentrated under reduced pressure to give the titled compound as a solid (181 mg, 100% yield).

LCMS (Method L): Rt=0.29; [M+H]+=267

### 3,5-dimethyl-2-[6-(9-methyl-1,9-diazaspiro[4.5]decan-1-yl)pyridazin-3-yl]phenol (Example 428)

In a microwave vial, under Ar, to a solution of (2-hydroxy-4,6-dimethylphenyl)boronic acid (0.618 mmol, 108 mg) and 1-(6-chloropyridazin-3-yl)-9-methyl-1,9-diazaspiro[4.5]decane (0.475 mmol, 127 mg) in 3 ml of dioxane were added sodium carbonate (1.43 mmol, 118 mg) and 0.74 ml of water. The solution was purged with Ar for 10 min, then Pd(PPh₃)₄ (23.8 μmol, 28 mg) was added. The vial was sealed, and the mixture was heated at 110° C. under microwave irradiation for 1 h. After cooling down to rt, the reaction mixture was filtered. The filtrate was concentrated under reduced pressure, then diluted with DCM and washed with water (3 times). The organic layer was dried over MgSO₄, filtered and concentrated under reduced pressure. The residue was purified by flash chromatography on silica gel using DCM/MeOH/ACN (from 100/0/0 to 96/2/2), then suspended in dichloromethane (1 mL) and stirred 10 min at room temperature. The suspension was filtered off and dried under reduced pressure to give the title compound as a white solid (13.9 mg, 8% yield).

Compounds Examples 440, 441, 444, 445, 446, 449, 450, 451 and 455 were synthesized analogously to Example 428. Chiral separation of Examples 445/446 and 450/451 were realized using Chiralpak® OD-I column.

### Example 452: 2-[6-[rac-(3aS,7aR)-6-methyl-3,3a,4,5,7,7a-hexahydro-2H-pyrrolo[2,3-c]pyridin-1-yl]pyridazin-3-yl]-3-ethyl-5-(trifluoromethyl)phenol

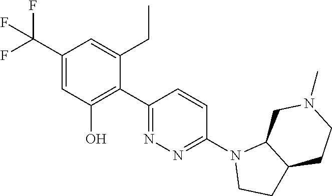 [SMILES: CCc1cc(C(F)(F)F)cc(O)c1-c1ccc(N2CC[C@@H]3CCN(C)C[C@@H]32)nn1]

In a microwave vial under Ar, to a mixture of 3-ethyl-2-(4,4,5,5-tetramethyl-1,3,2-dioxaborolan-2-yl)-5-(trifluoromethyl)phenol (Int 380.3-67.5 mg, 0.214 mmol), K₂CO₃ (49.2 mg, 0.356 mmol) and (3aS,7aR)-1-(6-chloropyridazin-3-yl)-6-methyl-3,3a,4,5,7,7a-hexahydro-2H-pyrrolo[2,3-c]pyridine (Int 342.1-45.0 mg, 0.178 mmol) in 1 ml of 1,4-dioxane and 0.2 ml of water were added XPhos (4.24 mg, 0.00890 mmol) and XPhos Pd G2 (7.00 mg, 0.0890 mmol). The mixture was heated at 90° C. for 3 h. After cooling down to rt, the mixture was filtrated over Celite® and concentrated under reduced pressure. The residue was purified by flash chromatography on silica gel using a gradient of DCM/(MeOH/NH₄OH (10%)) from 100/0 to 80/20). The residue obtained was purified by reverse preparative LC (Column YMC—Actus Triart Prep C18-S 150*30 mm 5 μm Flow rate 50 ml/min-Focused gradient MeCN/aq·NH₄HCO₃ 0.2% pH=7.9 Focused gradient from 30/70 to 65/35), to give after lyophilization the title compound (6 mg, 8% yield).

### Example 456: 2-[6-[(4aS,8aR)-6-ethyl-3,4a,5,7,8,8a-hexahydro-2H-pyrido[4,3-b][1,4]oxazin-4-yl]-4-(difluoromethyl)pyridazin-3-yl]-5-chloro-phenol

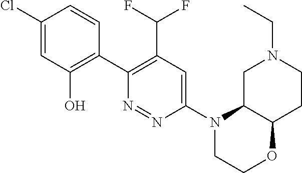 [SMILES: CCN1CC[C@H]2OCCN(c3cc(C(F)F)c(-c4ccc(Cl)cc4O)nn3)[C@H]2C1]

### Int 456.1: tert-butyl (4aS,8aR)-4-[6-(4-chloro-2-hydroxy-phenyl)-5-(difluoromethyl) pyridazin-3-yl]-3,4a,5,7,8,8a-hexahydro-2H-pyrido[4,3-b][1,4]oxazine-6-carboxylate

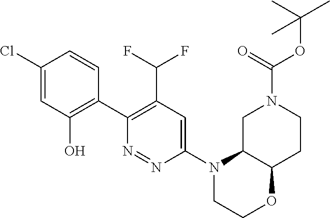 [SMILES: CC(C)(C)OC(=O)N1CC[C@H]2OCCN(c3cc(C(F)F)c(-c4ccc(Cl)cc4O)nn3)[C@H]2C1]

Under Ar, to a solution of (4-chloro-2-hydroxy-phenyl)boronic acid (1.11 mmol, 192 mg) and tert-butyl (4aS,8aR)-4-[6-chloro-5-(difluoromethyl)pyridazin-3-yl]-3,4a,5,7,8,8a-hexahydro-2H-pyrido[4,3-b][1,4]oxazine-6-carboxylate (Int 298.4-0.62 mmol, 250 mg) in 10 ml of 1,4-dioxane was added a 2N sodium carbonate aqueous solution (1.67 mmol, 834 μl), then tetrakis(triphenylphosphine)palladium(0) (0.031 mmol, 36 mg). The mixture was stirred at 100° C. for 2 h. After cooling down to rt, the mixture was diluted with 50 ml of EtOAc and 50 ml of diethylether and the organic layer was washed successively with 20 ml of a saturated aqueous sodium bicarbonate solution (twice) and 20 ml of water. The organic layer was dried over Na₂SO₄, filtered and concentrated under reduced pressure. The residue was purified by flash chromatography on silica gel eluting with a gradient of 0 to 60% of EtOAc in cyclohexane to give the title compound (306 mg, 99% yield) as a yellow solid.

LCMS (Method V): Rt=1.12; MS m/z [M+H]⁺=497

### Int 456.2: 2-[6-[(4aS,8aR)-2,3,4a,5,6,7,8,8a-octahydropyrido[4,3-b][1,4]oxazin-4-yl]-4-(difluoromethyl)pyridazin-3-yl]-5-chloro-phenol

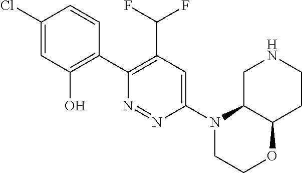 [SMILES: Oc1cc(Cl)ccc1-c1nnc(N2CCO[C@@H]3CCNC[C@@H]32)cc1C(F)F]

To a solution of tert-butyl (4aS,8aR)-4-[6-(4-chloro-2-hydroxy-phenyl)-5-(difluoromethyl)pyridazin-3-yl]-3,4a,5,7,8,8a-hexahydro-2H-pyrido[4,3-b][1,4]oxazine-6-carboxylate (0.616 mmol, 306 mg) in 9 ml of dichloromethane was added 1 ml of trifluoroacetic acid and the mixture was stirred for 2 h at rt, then concentrated under reduced pressure. The residue was diluted with 50 ml of dichloromethane and washed with 2×50 ml of a saturated aqueous sodium bicarbonate solution and 10 ml of water then dried over Na₂SO₄, filtered and concentrated under reduced pressure to give the title compound (244 mg, 100% yield) as a yellow solid.

LCMS (Method V): Rt=0.53; MS m/z [M+H]⁺=397

### 2-[6-[(4aS,8aR)-6-ethyl-3,4a,5,7,8,8a-hexahydro-2H-pyrido[4,3-b][1,4]oxazin-4-yl]-4-(difluoromethyl)pyridazin-3-yl]-5-chloro-phenol (Example 456)

To a solution of 2-[6-[(4aS,8aR)-2,3,4a,5,6,7,8,8a-octahydropyrido[4,3-b][1,4]oxazin-4-yl]-4-(difluoromethyl)pyridazin-3-yl]-5-chloro-phenol (0.61 mmol, 244 mg) and acetaldehyde (3.07 mmol, 135 mg) in 10 ml of DCM was added 0.528 ml of acetic acid and the mixture was stirred for 10 min at rt. Then was added sodium triacetoxyborohydride (1.11 mmol, 235 mg) and the mixture was stirred 18 h at rt. The reaction mixture was diluted with 50 ml of DCM and washed with 50 ml of a saturated aqueous sodium bicarbonate solution. The aqueous layer was extracted with 3×15 ml of dichloromethane and 2×15 ml of a mixture of dichloromethane/isopropanol: 90/10. The combined organic layers were dried over Na₂SO₄, filtered and concentrated under reduced pressure. The residue was purified by flash chromatography on silica gel using a gradient of DCM/MeOH/ACN (from 100/0/0 to 80/10/10), then repurified by prep-HPLC (column: Sunfire prep C18 OBD; 250*50 mm*10 μm; mobile phase: [aqueous TFA 10⁻² N/ACN] from 10% to 100% ACN in 35 min. at 50 ml/min) to give the title compound as a white solid (84 mg, 32% yield).

### Example 457: 1-[(3aR,7aR)-1-[6-(2-hydroxy-4-methyl-phenyl)-5-methyl-pyridazin-3-yl]-3,3a,4,5,7,7a-hexahydro-2H-pyrrolo[2,3-c]pyridin-6-yl]ethenone

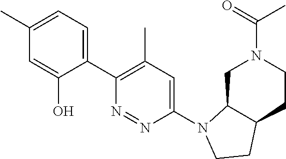 [SMILES: CC(=O)N1CC[C@H]2CCN(c3cc(C)c(-c4ccc(C)cc4O)nn3)[C@H]2C1]

### Int 457.1: tert-butyl (3aR,7aR)-1-(6-chloro-5-methyl-pyridazin-3-yl)-3,3a,4,5,7,7a-hexahydro-2H-pyrrolo[2,3-c]pyridine-6-carboxylate

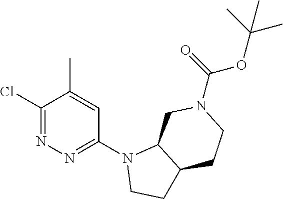 [SMILES: Cc1cc(N2CC[C@@H]3CCN(C(=O)OC(C)(C)C)C[C@@H]32)nnc1Cl]

In a microwave vial, under Ar, a solution of 3,6-dichloro-4-methyl-pyridazine (3.82 mmol, 635 mg), tert-butyl (3aR,7aR)-1,2,3,3a,4,5,7,7a-octahydropyrrolo[2,3-c]pyridine-6-carboxylate (2.94 mmol, 700 mg) and potassium phosphate tribasic (5.88 mmol, 1.25 g) in 20 ml of DMSO was heated at 100° C. under microwave irradiation for 2.5 h. After cooling down to rt, the reaction mixture was diluted with EtOAc and water. The organic layer was washed twice with water, with brine, then dried over Na₂SO₄, filtered and concentrated under reduced pressure. The residue was purified by flash chromatography on silica gel using a gradient of heptane/EtOAc (60/40) to give the title compound as a white solid (237 mg, 23% yield).

LCMS (Method L): Rt=0.94; MS m/z [M+H]⁺=353

### Int 457.2: (3aS,7aR)-1-(6-chloro-5-methyl-pyridazin-3-yl)-2,3,3a,4,5,6,7,7a-octahydropyrrolo[2,3-c]pyridine;hydrochloride

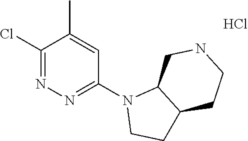 [SMILES: Cc1cc(N2CC[C@@H]3CCNC[C@@H]32)nnc1Cl ; Cl]

To a solution of tert-butyl (3aR,7aR)-1-(6-chloro-5-methyl-pyridazin-3-yl)-3,3a,4,5,7,7a-hexahydro-2H-pyrrolo[2,3-c]pyridine-6-carboxylate (0.67 mmol, 237 mg) in 5 ml of dichloromethane was added 1.68 ml of a 4N hydrochloric acid solution in 1,4-dioxane and the mixture was stirred at rt for 2 h, then concentrated under reduced pressure to give the title compound (193 mg) as an orange solid, which was used without further purification in the next step.

LCMS (Method L): Rt=0.29; [M+H]+=253

### Int 457.3: 1-[(3aR,7aR)-1-(6-chloro-5-methyl-pyridazin-3-yl)-3,3a,4,5,7,7a-hexahydro-2H-pyrrolo[2,3-c]pyridin-6-yl]ethanone

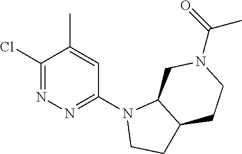 [SMILES: CC(=O)N1CC[C@H]2CCN(c3cc(C)c(Cl)nn3)[C@H]2C1]

Under Ar, to a suspension of (3aS,7aR)-1-(6-chloro-5-methyl-pyridazin-3-yl)-2,3,3a,4,5,6,7,7a-octahydropyrrolo[2,3-c]pyridine;hydrochloride (0.67 mmol, 193 mg) in 15 ml of DCM were added Et₃N (2.0 mmol, 280 μl), acetic anhydride (1.0 mmol, 95 μl) and DMAP (33 μmol, 4.2 mg). The mixture was stirred at rt for 3 h, then diluted with dichloromethane and washed with a saturated aqueous sodium bicarbonate solution. The organic layer was dried over Na₂SO₄, filtered and concentrated under reduced pressure. The residue was purified by flash chromatography on silica gel using a gradient of DCM/MeOH/ACN (95/2.5/2.5) to give the title compound as a white solid (75 mg, 38% yield).

LCMS (Method L): Rt=0.55; MS m/z [M+H]+=295

### 1-[(3aR,7aR)-1-[6-(2-hydroxy-4-methyl-phenyl)-5-methyl-pyridazin-3-yl]-3,3a,4,5,7,7a-hexahydro-2H-pyrrolo[2,3-c]pyridin-6-yl]ethenone (Example 457)

In a microwave vial, under Ar, to a solution of 1-[(3aR,7aR)-1-(6-chloro-5-methyl-pyridazin-3-yl)-3,3a,4,5,7,7a-hexahydro-2H-pyrrolo[2,3-c]pyridin-6-yl]ethanone (0.122 mmol, 36 mg) and 5-methyl-2-(4,4,5,5-tetramethyl-1,3,2-dioxaborolan-2-yl)-phenol (0.158 mmol, 38 mg) in 0.6 ml of 1,4-dioxane were added sodium carbonate (0.31 mmol, 33 mg) and 0.15 ml of water. The solution was purged with Ar, then Pd(PPh₃)₄ (6.1 μmol, 7 mg) was added. The vial was sealed, and the mixture was heated at 110° C. under microwave irradiation for 1 h. After cooling down to rt, the reaction was concentrated under reduced pressure. To the residue was added DCM and the organic layer was washed successively with a saturated aqueous sodium bicarbonate solution and brine. The organic layer was dried over Na₂SO₄, filtered and concentrated under reduced pressure. The residue was purified by flash chromatography on silica gel using a gradient of DCM/MeOH/ACN (95/2.5/2.5) to give the title compound as a white solid (22 mg, 63% yield).

Compound Example 514 was synthetized analogously to Example 457.

### Example 458: 2-[6-[rac-(3aS,7aR)-6-ethyl-3,3a,4,5,7,7a-hexahydro-2H-pyrrolo[2,3-c]pyridin-1-yl]pyridazin-3-yl]-3-(trifluoromethyl)phenol

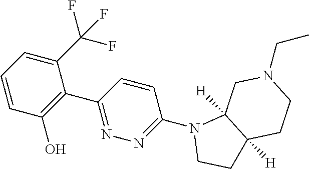 [SMILES: CCN1CC[C@H]2CCN(c3ccc(-c4c(O)cccc4C(F)(F)F)nn3)[C@H]2C1]

### Int 458.1: tert-butyl (3aR,7aR)-1-(6-chloropyridazin-3-yl)-3,3a,4,5,7,7a-hexahydro-2H-pyrrolo[2,3-c]pyridine-6-carboxylate

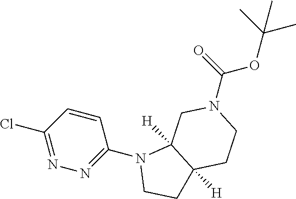 [SMILES: CC(C)(C)OC(=O)N1CC[C@H]2CCN(c3ccc(Cl)nn3)[C@H]2C1]

In a microwave vial, a mixture of tert-butyl (3aR,7aR)-1,2,3,3a,4,5,7,7a-octahydropyrrolo[2,3-c]pyridine-6-carboxylate (900 mg, 3.98 mmol), 3,6-dichloropyridazine (592 mg, 3.98 mmol) and K₃PO₄ (1.69 g, 7.95 mmol) in 13 ml of DMSO was heated at 80° C. using one single mode microwave (Biotage Initiator EXP 60) with a power output ranging from 0 to 400 W for 2 h. After cooling down to rt, the reaction mixture was filtered, then concentrated under reduced pressure. The residue was purified by flash chromatography on silica gel using a gradient of DCM/MeOH from 100/0 to 95/5, to give the title compound (776 mg, 57%).

LCMS (Method S) Rt=1.22; MS m/z [M+H]+=339.2

### Int 458.2: (3aS,7aR)-1-(6-chloropyridazin-3-yl)-2,3,3a,4,5,6,7,7a-octahydropyrrolo [2,3-c]pyridine;2,2,2-trifluoroacetic acid

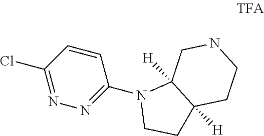 [SMILES: Clc1ccc(N2CC[C@@H]3CCNC[C@@H]32)nn1]

At 0° C., to a stirred solution of tert-butyl (3aR,7aR)-1-(6-chloropyridazin-3-yl)-3,3a,4,5,7,7a-hexahydro-2H-pyrrolo[2,3-c]pyridine-6-carboxylate (650 mg, 1.92 mmol) in 6 ml of dichloromethane was added TFA (1.42 mL, 19.2 mmol) and the reaction mixture was stirred at rt for 18 h. The reaction mixture was concentrated under reduced pressure to give the title compound (1.20 g), which was used without further purification in the next step.

1H NMR (400 MHz, DMSO-d6) δ (ppm): 8.45 (m, 1H), 7.57 (d, J=9.5 Hz, 1H), 7.05 (d, J=9.5 Hz, 1H), 4.56-4.17 (m, 1H), 3.72-3.46 (m, 2H), 3.46-3.26 (m, 1H), 3.24-3.07 (m, 1H), 3.00-2.81 (m, 2H), 2.50 (m, 1H), 2.27-2.10 (m, 1H), 2.09-1.93 (m, 2H), 1.89-1.81 (m, 1H).

### Int 458.3: (3aS,7aR)-1-(6-chloropyridazin-3-yl)-6-ethyl-3,3a,4,5,7,7a-hexahydro-2H-pyrrolo[2,3-c]pyridine

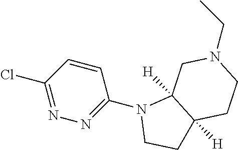 [SMILES: CCN1CC[C@H]2CCN(c3ccc(Cl)nn3)[C@H]2C1]

In sealed tube, to a stirred solution of (3aS,7aR)-1-(6-chloropyridazin-3-yl)-2,3,3a,4,5,6,7,7a-octahydropyrrolo[2,3-c]pyridine;2,2,2-trifluoroacetic acid (300 mg, 0.643 mmol) in 5 ml of ethanol were added acetaldehyde 5 M in THF (0.386 mL, 1.93 mmol), acetic acid (0.0368 mL, 0.643 mmol) and NaBH₃CN (80.8 mg, 1.29 mmol). The reaction mixture was stirred at rt for 1 h. The reaction mixture was concentrated under reduced pressure and the residue was purified by flash chromatography on silica gel using a gradient of DCM/(MeOH/NH₄OH (10%)) from 100% to 90/10, to give the title compound (135 mg, 80%).

LCMS (Method R): Rt=0.92; MS m/z [M+H]+=267.3

### 2-[6-[rac-(3aS,7aR)-6-ethyl-3,3a,4,5,7,7a-hexahydro-2H-pyrrolo[2,3-c]pyridin-1-yl]pyridazin-3-yl]-3-(trifluoromethyl)phenol (Example 458)

Under Ar, in a microwave vial, to a mixture of [[2-hydroxy-6-(trifluoromethyl)phenyl]boronic acid (74.1 mg, 0.360 mmol), K₂CO₃ (82.9 mg, 00.600 mmol), (3aS,7aR)-1-(6-chloropyridazin-3-yl)-6-ethyl-3,3a,4,5,7,7a-hexahydro-2H-pyrrolo[2,3-c]pyridine (80.0 mg, 0.300 mmol) in 2 ml of 1,4-dioxane and 0.5 ml of water were added XPhos (7.15 mg, 0.00150 mmol) and XPhos Pd G2 (11.8 mg, 0.00150 mmol). The mixture was purged again with Ar, then heated at 90° C. for 3 h. The reaction mixture was concentrated under reduced pressure and the residue was purified by flash chromatography on silica gel using a gradient of DCM/(MeOH/NH₄OH 90/10) 100/0 to 80/20, to give the title compound a white solid (80.8 mg, 68%).

### Example 473: 2-[6-[(3aS,7aR)-6-ethyl-3,3a,4,5,7,7a-hexahydro-2H-pyrrolo[2,3-c]pyridin-1-yl]pyridazin-3-yl]-3-(trifluoromethyl)phenol

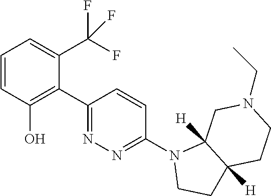 [SMILES: CCN1CC[C@@H]2CCN(c3ccc(-c4c(O)cccc4C(F)(F)F)nn3)[C@@H]2C1]

Both enantiomers of 2-[6-[rac-(3aS,7aR)-6-ethyl-3,3a,4,5,7,7a-hexahydro-2H-pyrrolo[2,3-c]pyridin-1-yl]pyridazin-3-yl]-3-(trifluoromethyl)phenol (Example 458-70 mg) were separated by Supercritical Fluidic Chromatography (SFC—Chiralpak® IC-3 4.6×100 mm, Mobile phase: CO₂/(MeOH+0.3% iPrNH2) 70/30)) to provide 2-[6-[(3aS,7aR)-6-ethyl-3,3a,4,5,7,7a-hexahydro-2H-pyrrolo[2,3-c]pyridin-1-yl]pyridazin-3-yl]-3-(trifluoromethyl) phenol, first enantiomer eluting at Rt=1.53 min (18 mg, Example 473, Enantiomer 1), as a white solid, and 2-[6-[(3aR,7aS)-6-ethyl-3,3a,4,5,7,7a-hexahydro-2H-pyrrolo[2,3-c]pyridin-1-yl]pyridazin-3-yl]-3-(trifluoromethyl)phenol, second enantiomer eluting at Rt=2.05 min.

The absolute configurations cannot be assigned at this stage.

### Example 459: 3-ethyl-5-methyl-2-[6-[rac-(3aS,7aR)-6-methyl-3,3a,4,5,7,7a-hexahydro-2H-pyrrolo[2,3-c]pyridin-1-yl]pyridazin-3-yl]phenol

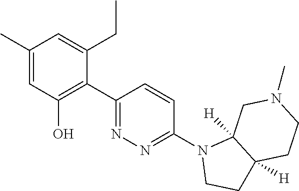 [SMILES: CCc1cc(C)cc(O)c1-c1ccc(N2CC[C@@H]3CCN(C)C[C@@H]32)nn1]

### Int 459.1: 3-[2-[tert-butyl(dimethyl)silyl]ethynyl]-5-methyl-phenol

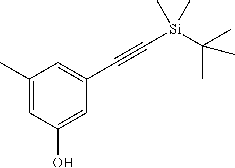 [SMILES: Cc1cc(O)cc(C#C[Si](C)(C)C(C)(C)C)c1]

To a solution of 3-bromo-5-methyl-phenol (2.00 g, 0.0107 mol) and tert-butyl-ethynyl-dimethyl-silane (5.99 mL 0.0321 mol) in 20 ml of DMF was added 4.5 ml of NEt₃ (4.5 mL, 32 mmol). The mixture was purged with N₂, then PdCl₂(dppf) (873 mg, 1.07 mol) and CuI (204 mg, 1.07 mmol) were added. The mixture was purged again with N₂ and stirred at 80° C. for 3 h. After cooling down to rt, the reaction mixture was concentrated under reduced pressure. The residue was purified by flash chromatography on silica gel using a gradient of heptane/EtOAc (100/0 to 80/20, then isocratic 80/20) to give the title compound as a dark orange residue (1.73 g, 66%).

1H NMR (400 MHz, DMSO) δ (ppm): 9.55 (s, 1H), 6.73-6.67 (m, 1H), 6.63-6.61 (m, 1H), 6.61-6.58 (m, 1H), 2.19 (s, 3H), 0.95 (s, 9H), 0.14 (s, 6H).

### Int 459.2: 3-[2-[tert-butyl(dimethyl)silyl]ethynyl]-2-iodo-5-methyl-phenol

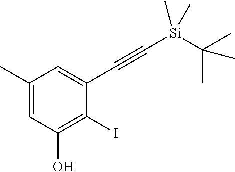 [SMILES: Cc1cc(O)c(I)c(C#C[Si](C)(C)C(C)(C)C)c1]

At 0° C., to a solution of 3-[2-[tert-butyl(dimethyl)silyl]ethynyl]-5-methyl-phenol (1.73 g, 0.00702 mol) in 70 ml of toluene) was added NaH (60% in mineral oil) (60.0%, 0.562 g, 0.0140 mol). The mixture was stirred for 10 min, then 12 (1.78 g, 0.00702 mol) was added. The reaction mixture was allowed to warm to rt and stirred for 18 h. The mixture was diluted with EtOAc, then quenched with a saturated aqueous solution of NH₄Cl. The aqueous layer was separated and extracted again with EtOAc. The combined organic layers were dried over MgSO₄, filtered and concentrated under reduced pressure. The residue was purified by flash chromatography on silica gel using a gradient of heptane/EtOAc (100/0 to 80/20, then isocratic 80/20), then purified by preparative chiral SFC (Stationary phase: Chiralpak® AD-3 4.6×100 mm, Mobile phase: CO₂/MeOH 90/10) to give the title compound as a yellow oil (173 mg, 7%).

1H NMR (400 MHz, DMSO) δ (ppm): 10.37 (s, 1H), 6.73 (d, J=1.8 Hz, 1H), 6.56 (d, J=1.9 Hz, 1H), 2.18 (s, 3H), 0.81 (s, 9H), 0.00 (s, 6H).

### Int 459.3: 3-ethynyl-2-iodo-5-methyl-phenol

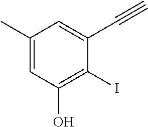

To a solution of 3-[2-[tert-butyl(dimethyl)silyl]ethynyl]-2-iodo-5-methyl-phenol (1.02 g, 0.00274 mol) in 27 ml of THF was added TBAF 1M in THF (4.11 mL, 0.00411 mol). The solution was stirred at rt for 5 h. EtOAc and water were added. The aqueous layer was separated and extracted with EtOAc. The combined organic layers were dried over MgSO₄, filtered and concentrated under reduced pressure. The residue was purified by flash chromatography on silica gel using a DCM to give the title compound as a pale pink oil which crystallized (0.585 g, 83%).

1H NMR (400 MHz, DMSO) δ (ppm): 10.28 (s, 1H), 6.65 (d, J=1.9 Hz, 1H), 6.52 (d, J=1.9 Hz, 1H), 4.24 (s, 1H), 2.00 (s, 3H).

### Int 459.4: 3-ethyl-2-iodo-5-methyl-phenol

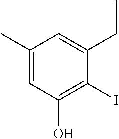

To a solution of 3-ethynyl-2-iodo-5-methyl-phenol (0.585 g, 0.00227 mol) in 25 ml of methanol was added Pd/C 10% activated on wood carbon, unreduced, 50% water wet paste (5.00%, 0.241 g, 0.000113 mol). The reaction was stirred under H₂ atmosphere (10 Bar) at rt for 5 h. The reaction mixture was filtered on a pad of Celite®, and the filtrate concentrated under reduced pressure. The residue was purified by flash chromatography on silica gel using a gradient of heptane/DCM (90/10 to 50/50) to give the title compound as a colorless oil (0.319 g, 54%).

1H NMR (400 MHz, DMSO) δ (ppm): 9.91 (s, 1H), 6.40 (d, J=1.9 Hz, 1H), 6.35 (d, J=2.0 Hz, 1H), 2.45 (q, J=7.5 Hz, 2H), 2.00 (s, 3H), 0.92 (t, J=7.5 Hz, 3H).

### Int 459.5: 3-ethyl-5-methyl-2-(4,4,5,5-tetramethyl-1,3,2-dioxaborolan-2-yl)phenol

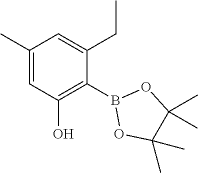

In a sealed tube, to a solution of 3-ethyl-2-iodo-5-methyl-phenol 2 (180 mg, 0.687 mol) and HBpin (0.50 mL, 3.44 mmol) in 8 ml of 1,4-dioxane was slowly added NEt₃ (0.670 mL, 4.81 mmol). After 1 min, the solution was purged with N₂. Pd(OAc)₂ (18.5 mg, 0.0824 mmol) and CyJohnPhos (53.0 mg, 0.151 mmol) were added. The mixture was purged again with N₂ then heated at 80° C. for 18 h. After cooling down to rt, water was added. The aqueous layer was separated and extracted twice with EtOAc. The combined organic layers were dried over MgSO₄, filtered and concentrated under reduced pressure. The residue was purified by flash chromatography on silica gel using a gradient of heptane/EtOAc (100/0 to 90/10) to give the title compound (93.4 mg, 51%).

1H NMR (500 MHz, DMSO-d6) δ (ppm): 8.81 (s, 1H), 6.44 (s, 1H), 6.40-6.35 (m, 1H), 2.59-2.50 (m, 2H), 2.17 (s, 3H), 1.29 (s, 12H), 1.08 (t, J=7.5 Hz, 3H)

### 3-ethyl-5-methyl-2-[6-[rac-(3aS,7aR)-6-methyl-3,3a,4,5,7,7a-hexahydro-2H-pyrrolo [2,3-c]pyridin-1-yl]pyridazin-3-yl]phenol (Example 459)

In a microwave vial, under N₂, to a solution of rac-(3aS,7aR)-1-(6-chloropyridazin-3-yl)-6-methyl-3,3a,4,5,7,7a-hexahydro-2H-pyrrolo[2,3-c]pyridine (Int 342.1-100 mg, 0.396 mmol) and 3-ethyl-5-methyl-2-(4,4,5,5-tetramethyl-1,3,2-dioxaborolan-2-yl)phenol (131 mg, 0.475 mmol) in 3 ml of 1,4-dioxane and 0.75 ml of water was added K₂CO₃ (109 mg, 0.000791 mol). The solution was degazed under N₂ flow. XPhos (9.43 mg, 1.98e-5 mol) and XPhos Pd G2 (15.6 mg, 1.98e-5 mol) were added. The reaction mixture was stirred at 90° C. for 4 h. After cooling down to rt, the mixture was filtrated and concentrated under reduced pressure. The residue was purified by flash chromatography on silica gel using a gradient DCM/(MeOH/NH₄OH: 90/10) 100/0 to 80/20 to give the title compound as a white solid (74 mg, 53%).

### Examples 476 and 477: 2-[6-[(3aR,7aS)-6-methyl-3,3a,4,5,7,7a-hexahydro-2H-pyrrolo[2,3-c]pyridin-1-yl]pyridazin-3-yl]-3-ethyl-5-methyl-phenol and 2-[6-[(3aS,7aR)-6-methyl-3,3a,4,5,7,7a-hexahydro-2H-pyrrolo[2,3-c]pyridin-1-yl]pyridazin-3-yl]-3-ethyl-5-methyl-phenol

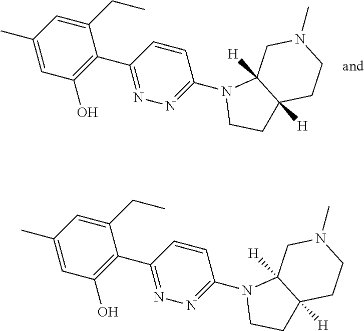 [SMILES: CCc1cc(C)cc(O)c1-c1ccc(N2CC[C@H]3CCN(C)C[C@H]32)nn1 ; CCc1cc(C)cc(O)c1-c1ccc(N2CC[C@@H]3CCN(C)C[C@@H]32)nn1]

Both enantiomers of 3-ethyl-5-methyl-2-[6-[rac-(3aS,7aR)-6-methyl-3,3a,4,5,7,7a-hexahydro-2H-pyrrolo[2,3-c]pyridin-1-yl]pyridazin-3-yl]phenol (Example 459-74 mg)) were separated by Supercritical Fluidic Chromatography (SFC—Column: Chiralpak® AD-H 20×250 mm, Mobile phase: CO₂/(MeOH+0.3% iPrNH₂) 70/30) to provide 2-[6-[(3aR,7aS)-6-methyl-3,3a,4,5,7,7a-hexahydro-2H-pyrrolo[2,3-c]pyridin-1-yl]pyridazin-3-yl]-3-ethyl-5-methyl-phenol, first enantiomer eluting at Rt=1.29 min (38 mg, Example 476, Enantiomer 1), as a white solid after lyophilization, and 2-[6-[(3aS,7aR)-6-methyl-3,3a,4,5,7,7a-hexahydro-2H-pyrrolo[2,3-c]pyridin-1-yl]pyridazin-3-yl]-3-ethyl-5-methyl-phenol, second enantiomer eluting at Rt=1.92 min (33 mg, Example 477, Enantiomer 2), as a white solid after lyophilization. The absolute configurations cannot be assigned at this stage.

### Example 460: 2-[6-[rac-(3aS,7aR)-6-ethyl-3,3a,4,5,7,7a-hexahydro-2H-pyrrolo[2,3-c]pyridin-1-yl]-4-methyl-pyridazin-3-yl]-5-methyl-phenol

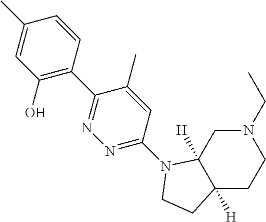 [SMILES: CCN1CC[C@H]2CCN(c3cc(C)c(-c4ccc(C)cc4O)nn3)[C@H]2C1]

### Int 460.1: tert-butyl rac-(3aR,7aR)-1-(6-chloro-5-methyl-pyridazin-3-yl)-3,3a,4,5,7,7a-hexahydro-2H-pyrrolo[2,3-c]pyridine-6-carboxylate

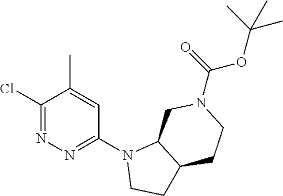 [SMILES: Cc1cc(N2CC[C@@H]3CCN(C(=O)OC(C)(C)C)C[C@@H]32)nnc1Cl]

In a microwave vial, a mixture of tert-butyl rac-(3aR,7aR)-1,2,3,3a,4,5,7,7a-octahydropyrrolo[2,3-c]pyridine-6-carboxylate (1.00 g, 4.42 mmol), 3,6-dichloro-4-methyl-pyridazine (720 mg, 4.42 mmol) and DIPEA (1.51 mL, 8.84 mmol) in 14 ml of butan-1-ol was heated at 130° C. for 10 h. After cooling down to tr, the reaction mixture was concentrated under reduced pressure. The residue was purified by flash chromatography on silica gel using a gradient of DCM/MeOH from 100/0 to 95/5, then repurified by preparative achiral SFC (Stationary phase: Cyano 20×250 mm, Mobile phase: CO₂/MeOH 95/5) to give tert-butyl rac-(3aR,7aR)-1-(6-chloro-4-methyl-pyridazin-3-yl)-3,3a,4,5,7,7a-hexahydro-2H-pyrrolo[2,3-c] pyridine-6-carboxylate as a pale-yellow solid (105 mg) and the title compound as a pale-yellow solid (433 mg, 28%).

LCMS (Method R): Rt=1.35; MS m/z [M+H]+=353.3

### Int 460.2: rac-(3aS,7aR)-1-(6-chloro-5-methyl-pyridazin-3-yl)-2,3,3a,4,5,6,7,7a-octahydropyrrolo[2,3-c]pyridine;2,2,2-trifluoroacetic acid

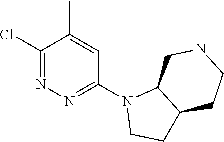 [SMILES: Cc1cc(N2CC[C@@H]3CCNC[C@@H]32)nnc1Cl]

At 0° C., to a stirred solution of tert-butyl rac-(3aR,7aR)-1-(6-chloro-5-methyl-pyridazin-3-yl)-3,3a,4,5,7,7a-hexahydro-2H-pyrrolo[2,3-c]pyridine-6-carboxylate (433 mg, 0.00123 mol) in 7 ml of dichloromethane was added TFA (0.912 mL, 12.3 mmol). The reaction mixture was then stirred at rt for 18 h. The reaction mixture was concentrated under reduced pressure to afford the title compound as a brown oil (1.04 g) which was used without further purification in next step.

1H NMR (500 MHz, DMSO-d6) δ (ppm): 8.39 (s, 1H), 7.00 (s, 1H), 4.40-4.32 (m, 1H), 3.56-3.49 (m, 2H), 3.36 (q, J=8.8 Hz, 1H), 3.19-3.11 (m, 1H), 3.09-3.01 (m, 1H), 2.99-2.90 (m, 1H), 2.58-2.52 (m, 1H), 2.28 (s, 3H), 2.22-2.11 (m, 1H), 2.09-1.93 (m, 2H), 1.89-1.81 (m, 1H), proton hidden under DMSO peak.

### Int 460.3: rac-(3aS,7aR)-1-(6-chloro-5-methyl-pyridazin-3-yl)-6-ethyl-3,3a,4,5,7,7a-hexahydro-2H-pyrrolo[2,3-c]pyridine

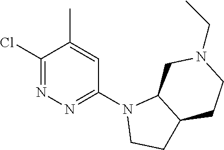 [SMILES: CCN1CC[C@H]2CCN(c3cc(C)c(Cl)nn3)[C@H]2C1]

To a stirred solution of rac-(3aS,7aR)-1-(6-chloro-5-methyl-pyridazin-3-yl)-2,3,3a,4,5,6,7,7a-octahydropyrrolo[2,3-c]pyridine;2,2,2-trifluoroacetic acid (520 mg, 1.08 mmol) in 12 ml of acetonitrile at rt were added K₂CO₃ (299 mg, 2.16 mmol) and iodoethane (0.0870 mL, 108 mmol). The reaction mixture was stirred at 50° C. for 5 h. After cooling down to rt, the reaction was concentrated under reduced pressure. The residue was purified by flash chromatography on silica gel using a gradient of DCM/(MeOH/NH₄OH: 90/10) from 100/0 to 95/5, to give the title compound as a pale-yellow oil (123 mg, 35%).

LCMS (Method T): Rt=5.25; MS m/z [M+H]+=281.2

### 2-[6-[rac-(3aS,7aR)-6-ethyl-3,3a,4,5,7,7a-hexahydro-2H-pyrrolo[2,3-c]pyridin-1-yl]-4-methyl-pyridazin-3-yl]-5-methyl-phenol (Example 460)

Under Ar, in a sealed tube, to a solution of rac-(3aS,7aR)-1-(6-chloro-5-methyl-pyridazin-3-yl)-6-ethyl-3,3a,4,5,7,7a-hexahydro-2H-pyrrolo[2,3-c]pyridine (118 mg, 0.420 mmol), K₂CO₃ (174 mg, 1.26 mmol) and (2-hydroxy-4-methyl-phenyl)boronic acid (95.8 mg, 0.630 mmol) in 2 ml of 1,4-dioxane and 0.5 ml of water were added XPhos Pd G2 (16.5 mg, 0.0210 mmol) and XPhos (10.0 mg, 0.0210 mmol). The mixture was purged again with Ar, then heated at 90° C. for 3.5 h. After cooling down to rt, the mixture was concentrated under reduced pressure. The residue was purified by flash chromatography on silica gel using a gradient of DCM/(MeOH/NH₄OH: 90/10) from 100/0 to 95/5, then repurified by reverse phase preparative LC (Column YMC—Actus Triart Prep C18-S 150*30 mm 5 μm Flow rate 50 ml/min-Focused gradient MeCN-MeOH [50/50]/aq. NH₄HCO₃ 0.2% pH=10 Focused gradient from 50/50 to 70/30 15CV) to give a pale yellow solid (56 mg, 38%). 9 mg were lyophilized to give the title compound as a white solid (9 mg).

### Example 468 and 469: 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-chloro-phenol and 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-chloro-phenol

In a microwave vial, under Ar, to a suspension of 4-chloro-2-hydroxyphenylboronic acid (487 μmol, 87 mg) and rac-(3aS,7aR)-1-(6-chloro-5-methyl-pyridazin-3-yl)-6-methyl-3,3a,4,5,7,7a-hexahydro-2H-pyrrolo[2,3-c]pyridine (375 μmol, 100 mg) in 1.9 ml of 1,4-dioxane and 0.5 ml of water was added sodium carbonate (937 μmol, 99 mg). The solution was purged with Ar for 5 min, then Pd(PPh₃)₄ (19 μmol, 22 mg) was added. The vial was sealed, and the mixture was heated at 110° C. under microwave irradiation for 1 h. After cooling down to rt, the reacting mixture was concentrated under reduced pressure. The residue was taken up with CH₂Cl₂, the organic layer was washed with water, saturated aqueous NaHCO₃ solution and brine, dried over Na₂SO₄, filtered and concentrated under reduced pressure. The residue was purified by flash chromatography on silica gel using DCM/ACN/MeOH (90/5/5)+1% NH₃ 7N in MeOH, then repurified by SFC (Column: Column Lux Amylose-1 5 μm, 250×30 mm, Flow rate 45 ml/min, eluent (Heptane/EtOH 80/20+0.1% TEA) to provide 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-chloro-phenol, first enantiomer eluting at Rt=9.7 min (44 mg, Example 468, Enantiomer 1), as a white solid, and 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-chloro-phenol, second enantiomer eluting at Rt=15.5 min (39 mg, Example 469, Enantiomer 2), as a white solid. The absolute configurations cannot be assigned at this stage.

### Examples 474 and 475: 2-[6-[(3aR,7aS)-6-ethyl-3,3a,4,5,7,7a-hexahydro-2H-pyrrolo[2,3-c]pyridin-1-yl]-4-methyl-pyridazin-3-yl]-5-methyl-phenol and 2-[6-[(3aS,7aR)-6-ethyl-3,3a,4,5,7,7a-hexahydro-2H-pyrrolo[2,3-c]pyridin-1-yl]-4-methyl-pyridazin-3-yl]-5-methyl-phenol

Both enantiomers of 2-[6-[rac-(3aS,7aR)-6-ethyl-3,3a,4,5,7,7a-hexahydro-2H-pyrrolo[2,3-c]pyridin-1-yl]-4-methyl-pyridazin-3-yl]-5-methyl-phenol (Example 460-47 mg) were separated by Supercritical Fluidic Chromatography (SFC—Chiralpak® IC-3 4.6×100 mm, Mobile phase: CO₂/(MeOH+0.3% iPrNH₂) 50/50) to provide 2-[6-[(3aR,7aS)-6-ethyl-3,3a,4,5,7,7a-hexahydro-2H-pyrrolo[2,3-c]pyridin-1-yl]-4-methyl-pyridazin-3-yl]-5-methyl-phenol, first enantiomer eluting at Rt=2.07 min (22 mg, Example 474, Enantiomer 1), as a white solid, and 2-[6-[(3aS,7aR)-6-ethyl-3,3a,4,5,7,7a-hexahydro-2H-pyrrolo[2,3-c]pyridin-1-yl]-4-methyl-pyridazin-3-yl]-5-methyl-phenol, second enantiomer eluting at Rt=2.68 min (20 mg, Example 475, Enantiomer 2), as a white solid. The absolute configurations cannot be assigned at this stage.

### Example 461: 2-[6-[(4aS,8aR)-6-methyl-3,4a,5,7,8,8a-hexahydro-2H-pyrido[4,3-b][1,4]oxazin-4-yl]pyridazin-3-yl]-5-chloro-3-ethyl-phenol

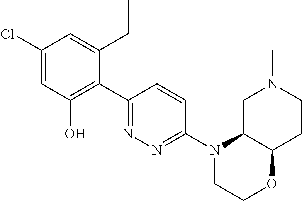 [SMILES: CCc1cc(Cl)cc(O)c1-c1ccc(N2CCO[C@@H]3CCN(C)C[C@@H]32)nn1]

Under Ar, to a solution of 5-chloro-3-ethyl-2-(4,4,5,5-tetramethyl-1,3,2-dioxaborolan-2-yl)phenol (Int 381.3 0.67 mmol, 189 mg) and (4aS,8aR)-4-(6-chloropyridazin-3-yl)-6-methyl-3,4a,5,7,8,8a-hexahydro-2H-pyrido[4,3-b][1,4]oxazine (Int 321.3-0.37 mmol, 100 mg) in 6 ml of 1,4-dioxane was added a 2N sodium carbonate aqueous solution (1 mmol, 500 μl), then tetrakis(triphenylphosphine)palladium(0) (0.019 mmol, 21 mg). The mixture was stirred at 100° C. for 1 h. After cooling down to rt, the mixture was diluted with 50 ml of EtOAc and 50 ml of diethylether and the organic layer was washed successively with 20 ml of a saturated aqueous sodium bicarbonate solution (twice) and 20 ml of water. The organic layer was dried over Na₂SO₄, filtered and concentrated under reduced pressure. The residue was purified by flash chromatography on silica gel using a gradient of DCM/MeOH/ACN (from 100/0/0 to 85/7.5/7.5) to give, after trituration in acetonitrile, the title compound as a white solid (48 mg, 33% yield).

Compound Example 526 was synthesized analogously to Example 461

### Example 471: 5-[6-[(4aS,8aR)-6-methyl-3,4a,5,7,8,8a-hexahydro-2H-pyrido[4,3-b][1,4]oxazin-4-yl]-4-methyl-pyridazin-3-yl]indan-4-ol

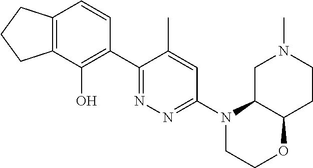 [SMILES: Cc1cc(N2CCO[C@@H]3CCN(C)C[C@@H]32)nnc1-c1ccc2c(c1O)CCC2]

Under Ar, to a solution of 5-(4,4,5,5-tetramethyl-1,3,2-dioxaborolan-2-yl)indan-4-ol (Int 324.2-1.99 mmol, 519 mg) and (4aS,8aR)-4-(6-chloro-5-methyl-pyridazin-3-yl)-6-methyl-3,4a,5,7,8,8a-hexahydro-2H-pyrido[4,3-b][1,4]oxazine (Int 373.3-0.84 mmol, 238 mg) in 8 ml of 1,4-dioxane and 1.5 ml of water was added a 2N sodium carbonate aqueous solution (2.53 mmol, 1.26 ml), then tetrakis(triphenylphosphine)palladium(0) (0.084 mmol, 97 mg). The mixture was stirred at 100° C. for 1.5 h. After cooling down to rt, the mixture was diluted with 50 ml of DCM and washed successively with 20 ml of a 2N aqueous sodium carbonate solution and 20 ml of a saturated aqueous sodium bicarbonate solution. The pooled aqueous layers were extracted with 20 ml of DCM and the combined organic layers were dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 89/5/5/1 to give the title compound as a pale yellow solid (196 mg, 61% yield).

### Example 472: 2-[6-[(4aS,8aR)-6-isopropyl-3,4a,5,7,8,8a-hexahydro-2H-pyrido[4,3-b][1,4]oxazin-4-yl]-4-methyl-pyridazin-3-yl]-5-methyl-phenol

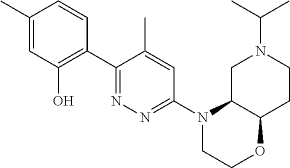 [SMILES: Cc1ccc(-c2nnc(N3CCO[C@@H]4CCN(C(C)C)C[C@@H]43)cc2C)c(O)c1]

### Int 472.1: (4aS,8aR)-4-(6-chloro-5-methyl-pyridazin-3-yl)-6-isopropyl-3,4a,5,7,8,8a-hexahydro-2H-pyrido[4,3-b][1,4]oxazine

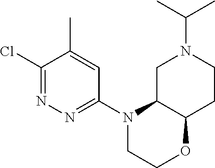 [SMILES: Cc1cc(N2CCO[C@@H]3CCN(C(C)C)C[C@@H]32)nnc1Cl]

To a solution of tert-butyl (4aS,8aR)-4-(6-chloro-5-methyl-pyridazin-3-yl)-3,4a,5,7,8,8a-hexahydro-2H-pyrido[4,3-b][1,4]oxazine-6-carboxylate (Int 373.1-0.76 mmol, 280 mg) in 8 ml of dichloromethane was added 2 ml of trifluoroacetic acid and the mixture was stirred at RT for 4 h, then concentrated under reduced pressure. To the residue solubilized in 15 ml of dichloromethane were added successively acetone (19 mmol, 1.4 ml), and sodium triacetoxyborohydride (1.37 mmol, 290 mg) and the mixture was stirred at rt for 2 h. Then were added successively triethylamine (4.55 mmol, 635 μl), acetic acid (3.8 mmol, 217 μl), 1 ml of MeOH and another sodium triacetoxyborohydride (1.52 mmol, 322 mg) and the mixture was stirred again at rt for 1 h30. The reaction mixture was then diluted with 10 ml of dichloromethane and 5 ml of a saturated aqueous sodium bicarbonate solution. The aqueous layer was extracted with 10 ml of dichloromethane and the combined organic layers were dried over Na₂SO₄, filtered and concentrated under reduced pressure. To the residue solubilized in 15 ml of dichloromethane and 2 ml of MeOH were added 440 μl of acetic acid, acetone (19 mmol, 1.4 ml) and sodium triacetoxyborohydride (4.56 mmol, 1.68 g). The reaction mixture was stirred at rt for 1 h15 then was added another sodium triacetoxyborohydride (4.56 mmol, 1.68 g) and the mixture was stirred at rt for 1 h. The reaction mixture was then diluted with 20 ml of dichloromethane, 10 ml of a 2N aqueous sodium carbonate and 10 ml of a saturated aqueous sodium bicarbonate solution. The aqueous layer was extracted with 10 ml of dichloromethane and the combined organic layers were dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 89/5/5/1 to give the title compound as a pale yellow foam (131 mg, 56% yield).

LCMS (Method L): Rt=0.53; MS m/z [M+H]⁺=311

### 2-[6-[(4aS,8aR)-6-isopropyl-3,4a,5,7,8,8a-hexahydro-2H-pyrido[4,3-b][1,4]oxazin-4-yl]-4-methyl-pyridazin-3-yl]-5-methyl-phenol (Example 472)

Under Ar, to a solution of 5-methyl-2-(4,4,5,5-tetramethyl-1,3,2-dioxaborolan-2-yl)phenol (0.84 mmol, 196 mg) and (4aS,8aR)-4-(6-chloro-5-methyl-pyridazin-3-yl)-6-isopropyl-3,4a,5,7,8,8a-hexahydro-2H-pyrido[4,3-b][1,4]oxazine (0.42 mmol, 130 mg) in 6 ml of 1,4-dioxane and 0.5 ml of water was added a 2N sodium carbonate aqueous solution (1.25 mmol, 627 μl), then tetrakis(triphenylphosphine)palladium(0) (0.042 mmol, 48 mg). The mixture was stirred at 100° C. for 3 h. After cooling down to rt, the mixture was diluted with 50 ml of DCM and washed successively with 10 ml of a 2N aqueous sodium carbonate solution and 20 ml of a saturated aqueous sodium bicarbonate solution. The pooled aqueous layers were extracted with 20 ml of DCM and the combined organic layers were dried over Na₂SO₄, filtered and concentrated under reduced pressure. The residue was purified by flash chromatography on silica gel using a gradient of DCM/MeOH/ACN/NH₃ 7N sol. in MeOH (from 100/0/0/0 to 89/5/5/1 to give the title compound as a pale yellow solid (105 mg, 66% yield).

### Example 480: 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-fluoro-phenol

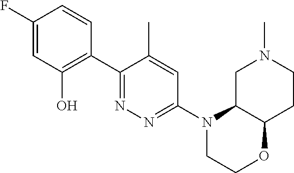 [SMILES: Cc1cc(N2CCO[C@@H]3CCN(C)C[C@@H]32)nnc1-c1ccc(F)cc1O]

Under Ar, to a solution of 5-fluoro-2-(4,4,5,5-tetramethyl-1,3,2-dioxaborolan-2-yl)phenol (0.94 mmol, 225 mg) and (4aS,8aR)-4-(6-chloro-5-methyl-pyridazin-3-yl)-6-methyl-3,4a,5,7,8,8a-hexahydro-2H-pyrido[4,3-b][1,4]oxazine (Int 373.3-0.63 mmol, 178 mg) in 6 ml of 1,4-dioxane was added a 2N sodium carbonate aqueous solution (1.70 mmol, 850 μl), then TETRAKIS(triphenylphosphine)palladium(0) (0.0315 mmol, 36 mg). The mixture was stirred at 100° C. for 2 h. After cooling down to rt, the mixture was diluted with 50 ml of EtOAc and 50 ml of diethylether and the organic layer was washed successively with 20 ml of a saturated aqueous sodium bicarbonate solution (twice) and 20 ml of water. The organic layer was dried over Na₂SO₄, filtered and concentrated under reduced pressure. The residue was purified by flash chromatography on silica gel using a gradient of DCM/MeOH/ACN (from 100/0/0 to 85/7.5/7.5), then repurified by prep-HPLC (column: Sunfire prep C18 OBD; 250*50 mm*10 μm; mobile phase: [aqueous TFA 10⁻² N/ACN] from 10% to 100% ACN in 35 min. at 50 ml/min) to give the title compound as a white solid (52 mg, 22% yield).

Compound Example 479 was synthesized analogously to Example 480 starting from Int435.1.

### Example 481: 2-[6-[(4aS,8aR)-6-methyl-3,4a,5,7,8,8a-hexahydro-2H-pyrido[4,3-b][1,4]oxazin-4-yl]pyridazin-3-yl]-3-ethyl-5-(trifluoromethyl)phenol

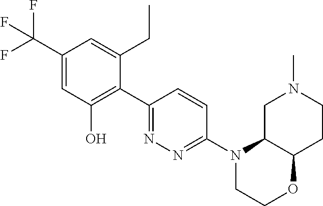 [SMILES: CCc1cc(C(F)(F)F)cc(O)c1-c1ccc(N2CCO[C@@H]3CCN(C)C[C@@H]32)nn1]

Under Ar, to a solution of 3-ethyl-2-(4,4,5,5-tetramethyl-1,3,2-dioxaborolan-2-yl)-5-(trifluoromethyl)phenol (Int 380.3-0.59 mmol, 176 mg) and (4aS,8aR)-4-(6-chloropyridazin-3-yl)-6-methyl-3,4a,5,7,8,8a-hexahydro-2H-pyrido[4,3-b][1,4]oxazine (Int 321.3-0.37 mmol, 100 mg) in 6 ml of 1,4-dioxane was added a 2N sodium carbonate aqueous solution (1 mmol, 500 μl), then tetrakis(triphenylphosphine)palladium(0) (0.019 mmol, 21 mg). The mixture was stirred at 100° C. for 2 h. After cooling down tort, the mixture was diluted with 50 ml of EtOAc and 50 ml of diethylether and the organic layer was washed successively with 20 ml of a saturated aqueous sodium bicarbonate solution (twice) and 20 ml of water. The organic layer was dried over Na₂SO₄, filtered and concentrated under reduced pressure. The residue was purified by flash chromatography on silica gel using a gradient of DCM/MeOH/ACN (from 100/0/0 to 85/7.5/7.5) to give, after trituration in acetonitrile, the title compound as a white solid (57 mg, 36% yield).

### Examples 482 and 483: 2-[6-[(3aS,7aR)-6-methyl-3,3a,4,5,7,7a-hexahydro-2H-pyrrolo[2,3-c]pyridin-1-yl]-4-(difluoromethyl)pyridazin-3-yl]-5-(trifluoromethyl)phenol and 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-(trifluoromethyl)phenol

### Int 482.1: 2-[4-(difluoromethyl)-6-[rac-(3aS,7aR)-6-methyl-3,3a,4,5,7,7a-hexahydro-2H-pyrrolo[2,3-c]pyridin-1-yl]pyridazin-3-yl]-5-(trifluoromethyl)phenol

In a microwave vial, under Ar, to a solution of rac-(3aS,7aR)-1-[6-chloro-5-(difluoromethyl)pyridazin-3-yl]-6-methyl-3,3a,4,5,7,7a-hexahydro-2H-pyrrolo[2,3-c]pyridine (Int 506.3; 0.51 mmol, 155 mg) 2-hydroxy-4-(trifluoromethyl)phenyl boronic acid (0.77 mmol, 158 mg) in 3.2 ml of 1,2-dimethoxyethane were added sodium carbonate (1.28 mmol, 136 mg) and 0.7 ml of water. The solution was purged with Ar, then Pd(PPh₃)₄ (0.04 mmol, 42 mg) was added. The vial was sealed, and the mixture was heated at 100° C. under microwave irradiation for 1 h. The reaction mixture was diluted with EtOAc, filtered and concentrated under reduce pressure. The residue was diluted with CH₂Cl₂, filtered and concentrated under reduce pressure. The crude product was purified by flash chromatography on silica gel using DCM/MeOH/ACN (from 100/0/0 to 80/10/10) to give the title compound as a pale yellow solid (75 mg, 34% yield).

LCMS (Method U): Rt=1.06; MS m/z [M+H]⁺=429

### 2-[6-[(3aS,7aR)-6-methyl-3,3a,4,5,7,7a-hexahydro-2H-pyrrolo[2,3-c]pyridin-1-yl]-4-(difluoromethyl)pyridazin-3-yl]-5-(trifluoromethyl)phenol and 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-(trifluoromethyl)phenol (Examples 482 et 483)

Both enantiomers of 2-[4-(difluoromethyl)-6-[rac-(3aS,7aR)-6-methyl-3,3a,4,5,7,7a-hexahydro-2H-pyrrolo[2,3-c]pyridin-1-yl]pyridazin-3-yl]-5-(trifluoromethyl)phenol (74 mg) were separated by chiral preparative HPLC (Column Chiralpak® AD 5 μm, 300×50 mm, Flow rate 230 ml/min, eluent (CO₂/ethanol+0.1% TEA 85/15), to provide 2-[6-[(3aS,7aR)-6-methyl-3,3a,4,5,7,7a-hexahydro-2H-pyrrolo[2,3-c]pyridin-1-yl]-4-(difluoromethyl) pyridazin-3-yl]-5-(trifluoromethyl)phenol, first enantiomer eluting Rt=7.0 min (29 mg, Example 482), as a white solid and 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-methyl-phenol, second enantiomer eluting at Rt=10.25 min (27 mg, Example 483), as a white solid. The absolute configurations cannot be assigned at this stage.

### Example 484: 3-methyl-2-[6-[rac-(3aS,6aS)-1-methyl-2,3,3a,5,6,6a-hexahydropyrrolo [3,2-b]pyrrol-4-yl]pyridazin-3-yl]-5-(trifluoromethyl)phenol

### Int 484.1: rac-(3aS,6aS)-4-(6-chloropyridazin-3-yl)-1-methyl-2,3,3a,5,6,6a-hexahydropyrrolo[3,2-b]pyrrole

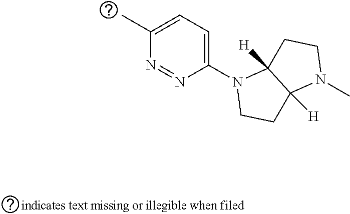

Under vigorous stirring, at rt, to a solution of rac-(3aS,6aS)-4-(6-chloropyridazin-3-yl)-2,3,3a,5,6,6a-hexahydro-1H-pyrrolo[3,2-b]pyrrole; dihydrochloride (Int 377.2; 270 mg, 0.9072 mmol) and Formalin (184 mg, 2.2681 mmol, 37 mass %) in 15 ml of DCM was added by fractions sodium triacetoxyborohydride (769 mg, 3.6289 mmol). The reaction was stirred at rt overnight. The reaction mixture was diluted with DCM, washed with an aqueous saturated solution of NaHCO₃ and brine, dried over MgSO₄, filtered and concentrated under reduce pressure to give the title compound as a solid (133 mg, 61% yield).

LCMS (Method L): Rt=0.23 min; MS m/z [M+H]+=239

### 3-methyl-2-[6-[rac-(3aS,6aS)-1-methyl-2,3,3a,5,6,6a-hexahydropyrrolo[3,2-b]pyrrol-4-yl]pyridazin-3-yl]-5-(trifluoromethyl)phenol (Example 484)

In a microwave vial, under Ar, to a solution of rac-(3aS,6aS)-4-(6-chloropyridazin-3-yl)-1-methyl-2,3,3a,5,6,6a-hexahydropyrrolo[3,2-b]pyrrole (133 mg, 0.5571 mmol) and aqueous solution of sodium carbonate (2 mol/l; 0.750 ml, 1.5043 mmol) in 4 ml of 1,4-dioxane were added 3-methyl-2-(4,4,5,5-tetramethyl-1,3,2-dioxaborolan-2-yl)-5-(trifluoromethyl)phenol (253 mg, 0.8357 mmol) and tetrakis(triphenylphosphine)palladium(0) (64 mg, 0.0557 mmol). The mixture was heated at 100° C. under microwave irradiation for 2 h. After cooling down to rt, the reaction mixture was diluted with AcOEt, extracted with water and brine, dried over MgSO₄, filtered and concentrated under reduced pressure. The residue was purified by flash chromatography on silica gel eluting with a gradient of DCM/[DCM80/CH₃CN 10/MeOH 10/Et₃N 0.1] (from 100/0 to 0/100), then repurified by chiral phase LC (Column WHELK 0®1 SS 5 μm, 30×250 mm, Flow rate 45 ml/min, eluent n-heptane/ethanol (70/30)+0.1% TEA, detection 265 nm) to give the title compound as a white solid (58 mg, 25% yield).

### Examples 487 and 488: 2-[6-[(3aR,7aS)-6-methyl-3,3a,4,5,7,7a-hexahydro-2H-pyrrolo[2,3-c]pyridin-1-yl]-5-(difluoromethyl)pyridazin-3-yl]-3,5-dimethyl-phenol and 2-[6-[(3aS,7aR)-6-methyl-3,3a,4,5,7,7a-hexahydro-2H-pyrrolo[2,3-c]pyridin-1-yl]-5-(difluoromethyl)pyridazin-3-yl]-3,5-dimethyl-phenol

### Int 487.1 and Int 487.1bis: tert-butyl rac-(3aR,7aR)-1-[6-chloro-5-(difluoromethyl)pyridazin-3-yl]-3,3a,4,5,7,7a-hexahydro-2H-pyrrolo[2,3-c]pyridine-6-carboxylate (Int 487.1) and tert-butyl rac-(3aR,7aR)-1-[6-chloro-4-(difluoromethyl)pyridazin-3-yl]-3,3a,4,5,7,7a-hexahydro-2H-pyrrolo[2,3-c]pyridine-6-carboxylate (Int 487.1bis)

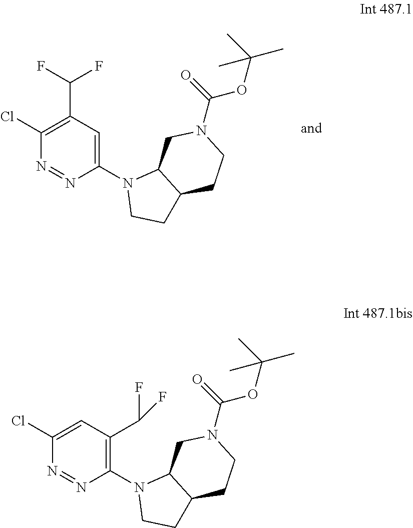 [SMILES: CC(C)(C)OC(=O)N1CC[C@H]2CCN(c3cc(C(F)F)c(Cl)nn3)[C@H]2C1 ; CC(C)(C)OC(=O)N1CC[C@H]2CCN(c3nnc(Cl)cc3C(F)F)[C@H]2C1]

In a microwave vial, a solution of 3,6-dichloro-4-(difluoromethyl)pyridazine (0.100 g, 0.000503 mol), tert-butyl rac-(3aR,7aR)-1,2,3,3a,4,5,7,7a-octahydropyrrolo[2,3-c]pyridine-6-carboxylate (0.114 g, 0.000503 mol) and K₃PO₄ (0.213 g, 0.00101 mol) in 1 ml of DMSO was heated at 80° C. using a single mode microwave (Biotage Initiator EXP 60) with a power output ranging from 0 to 400 W for 2 h. After cooling down to rt, the mixture was filtered. The solid was rinsed with DCM and the filtrate was concentrated under reduced pressure. The residue was purified by flash chromatography on silica gel using a gradient heptane/EtOAc (90/10 to 70/30, then isocratic) to give Int 487.1bis as a yellow solid (67.6 mg, 35%) and Int 487.1 as a yellow solid (0.106 g, 54%).

### Int 487.1:

1H NMR (400 MHz, DMSO) δ (ppm): 7.13 (t, J=53.3 Hz, 1H), 7.12 (s, 1H), 4.28-3.69 (m, 2H), 3.66-3.38 (m, 3H), 3.29-2.70 (m, 2H), 2.31-1.91 (m, 2H), 1.81 (s, 1H), 1.63 (s, 1H), 1.51-1.21 (m, 10H).

### Int 487.1bis:

1H NMR (400 MHz, DMSO) δ (ppm): 7.84 (s, 1H), 7.26 (t, J=53.2 Hz, 1H), 4.44 (d, J=14.6 Hz, 1H), 4.29-4.02 (m, 1H), 3.92-3.71 (m, 2H), 3.41 (t, J=8.9 Hz, 1H), 3.13 (d, J=14.8 Hz, 1H), 2.78 (t, J=12.2 Hz, 1H), 2.45-2.38 (m, 1H), 2.08-1.88 (m, 1H), 1.79-1.56 (m, 2H), 1.51-1.27 (m, 4H), 1.01 (s, 6H)

### Int 487.2: rac-(3aS,7aR)-1-[6-chloro-4-(difluoromethyl)pyridazin-3-yl]-2,3,3a,4,5,6,7,7a-octahydropyrrolo[2,3-c]pyridine;2,2,2-trifluoroacetic acid

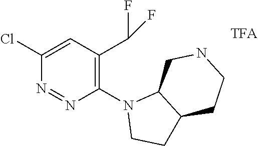 [SMILES: FC(F)c1cc(Cl)nnc1N1CC[C@@H]2CCNC[C@@H]21]

To a solution of tert-butyl rac-(3aR,7aR)-1-[6-chloro-4-(difluoromethyl)pyridazin-3-yl]-3,3a,4,5,7,7a-hexahydro-2H-pyrrolo[2,3-c]pyridine-6-carboxylate (Int 487.1bis—200 mg, 0.000514 mol) in 3.5 ml of DCM was added TFA (0.586 g, 0.00514 mol) and the solution was stirred at rt for 4 h. The mixture was concentrated under reduced pressure to give the title compound as a brown oil (241 mg) which was used without further purification in the next step.

LCMS (Method S): MS m/z [M+H]+=289; Rt=0.85

### Int 487.3: rac-(3aS,7aR)-1-[6-chloro-4-(difluoromethyl)pyridazin-3-yl]-6-methyl-3,3a,4,5,7,7a-hexahydro-2H-pyrrolo[2,3-c]pyridine

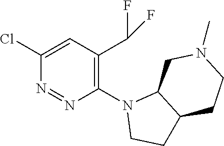 [SMILES: CN1CC[C@H]2CCN(c3nnc(Cl)cc3C(F)F)[C@H]2C1]

To a solution of rac-(3aS,7aR)-1-[6-chloro-4-(difluoromethyl)pyridazin-3-yl]-2,3,3a,4,5,6,7,7a-octahydropyrrolo[2,3-c]pyridine;2,2,2-trifluoroacetic acid (200 mg, 0.000387 mol) in 1.5 ml of methanol and 1.5 ml of tetrahydrofuran were added formaldehyde (37% in water, 0.157 g, 0.00194 mol) and NaBH₃CN (1 eq., 0.0243 g, 0.000387 mol). After stirring at rt for 2 h, the resulting solution was under reduced pressure. The residue was purified by flash chromatography on silica gel using a gradient DCM/MeOH (100/0 to 80/20) to give the title compound as a yellow oil (110 mg, 77%). 1H NMR (400 MHz, CDCl₃) δ (ppm): 7.62 (d, J=1.6 Hz, 1H), 7.11 (t, J=53.3 Hz, 1H), 4.67 (dt, J=5.9, 3.0 Hz, 1H), 3.93 (td, J=9.9, 6.6 Hz, 1H), 3.75-3.37 (m, 3H), 2.84 (dd, J=13.4, 3.4 Hz, 1H), 2.71-2.42 (m, 5H), 2.16-2.01 (m, 2H), 2.01-1.90 (m, 1H), 1.90-1.82 (m, 1H).

### 2-[6-[(3aR,7aS)-6-methyl-3,3a,4,5,7,7a-hexahydro-2H-pyrrolo[2,3-c]pyridin-1-yl]-5-(difluoromethyl)pyridazin-3-yl]-3,5-dimethyl-phenol and 2-[6-[(3aS,7aR)-6-methyl-3,3a,4,5,7,7a-hexahydro-2H-pyrrolo[2,3-c]pyridin-1-yl]-5-(difluoromethyl)pyridazin-3-yl]-3,5-dimethyl-phenol (Examples 487 and 488)

To rac-(3aS,7aR)-1-[6-chloro-4-(difluoromethyl)pyridazin-3-yl]-6-methyl-3,3a,4,5,7,7a-hexahydro-2H-pyrrolo[2,3-c]pyridine (90.0 mg, 0.000297 mol), (2-hydroxy-4,6-dimethyl-phenyl)boronic acid (0.0740 g, 0.000446 mol) were added potassium carbonate (0.123 g, 0.000892 mol), XPhos Pd G2 (0.0117 g, 1.49e-5 mol) and XPhos (0.00709 g, 1.49e-5 mol). The resulting mixture was purged under vacuum and backfilled with nitrogen 3 times, then 1.8 ml of 1,4-dioxane and 0.5 ml of water were added, and the resulting mixture was stirred at 90° C. for 18 h. After cooling down to rt, the resulting mixture was concentrated under reduced pressure. The residue was purified by flash chromatography on silica gel using a gradient DCM/MeOH (100/0 to 30/70) then repurified by reverse phase preparative LC (spherical C18 25 μm, 12 g YMC-ODS-25, dry loading (Celite®), mobile phase gradient 0.2% aq. NH₄+HCO₃—/MeCN from 75:25 to 40:60) to give 26 mg of a white solid. This solid was purified by SFC (Stationary phase: Chiralpak® IG 20×250 mm, Mobile phase: CO₂/(MeOH+0.3% iPrNH₂) 65/35) to provide 2-[6-[(3aR,7aS)-6-methyl-3,3a,4,5,7,7a-hexahydro-2H-pyrrolo[2,3-c]pyridin-1-yl]-5-(difluoromethyl)pyridazin-3-yl]-3,5-dimethyl-phenol, first enantiomer eluting Rt=1.05 min (9 mg, Example 487), as a white solid and 2-[6-[(3aS,7aR)-6-methyl-3,3a,4,5,7,7a-hexahydro-2H-pyrrolo[2,3-c]pyridin-1-yl]-5-(difluoromethyl)pyridazin-3-yl]-3,5-dimethyl-phenol, second enantiomer eluting at Rt=2.26 min (14 mg, Example 488), as a white solid. The absolute configurations cannot be assigned at this stage.

### Examples 489 and 490: 2-[6-[(3aR,7aS)-6-ethyl-3,3a,4,5,7,7a-hexahydro-2H-pyrrolo[2,3-c]pyridin-1-yl]-4-(difluoromethyl)pyridazin-3-yl]-5-methyl-phenol and 2-[6-[(3aS,7aR)-6-ethyl-3,3a,4,5,7,7a-hexahydro-2H-pyrrolo[2,3-c]pyridin-1-yl]-4-(difluoromethyl) pyridazin-3-yl]-5-methyl-phenol

### Int 489.1: rac-(3aS,7aR)-1-[6-chloro-5-(difluoromethyl)pyridazin-3-yl]-2,3,3a,4,5,6,7,7a-octahydropyrrolo[2,3-c]pyridine;2,2,2-trifluoroacetic acid

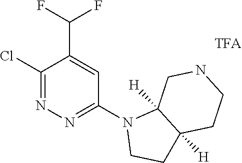 [SMILES: FC(F)c1cc(N2CC[C@@H]3CCNC[C@@H]32)nnc1Cl]

At 0° C., to a solution of tert-butyl rac-(3aR,7aR)-1-[6-chloro-5-(difluoromethyl)pyridazin-3-yl]-3,3a,4,5,7,7a-hexahydro-2H-pyrrolo[2,3-c]pyridine-6-carboxylate (Int 506.1-0.67 mmol, 260 mg) in 5 ml of dichloromethane was added TFA (6.7 mmol, 0.762 g). The reaction mixture was stirred at rt for 18 h, then concentrated under reduced pressure to give the title compound (330 mg) as a brown oil, which was used without further purification in the next step.

LCMS (Method S): Rt=0.84; MS m/z [M+H]⁺=289

### Int 489.2: rac-(3aS,7aR)-1-[6-chloro-5-(difluoromethyl)pyridazin-3-yl]-6-ethyl-3,3a,4,5,7,7a-hexahydro-2H-pyrrolo[2,3-c]pyridine

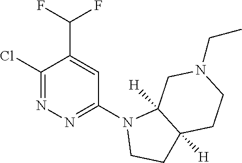 [SMILES: CCN1CC[C@H]2CCN(c3cc(C(F)F)c(Cl)nn3)[C@H]2C1]

To a solution of rac-(3aS,7aR)-1-[6-chloro-5-(difluoromethyl)pyridazin-3-yl]-2,3,3a,4,5,6,7,7a-octahydropyrrolo[2,3-c]pyridine;2,2,2-trifluoroacetic acid (0.621 mmol, 250 mg) in 4 ml of acetonitrile were added K₂CO₃ (2.48 mmol, 0.343 g) followed by iodoethane (0.621 mmol, 0.097 g). The mixture was stirred at 50° C. for 3 h. The reaction mixture was concentrated under reduced pressure. The residue was purified by flash chromatography on silica gel using a gradient of DCM/MeOH (from 100/0 to 90/10) to give the title compound as a yellow oil (260 mg, 94%)

LCMS (Method S): Rt=1.00; MS m/z [M+H]⁺=317

### 2-[6-[(3aR,7aS)-6-ethyl-3,3a,4,5,7,7a-hexahydro-2H-pyrrolo[2,3-c]pyridin-1-yl]-4-(difluoromethyl)pyridazin-3-yl]-5-methyl-phenol and 2-[6-[(3aS,7aR)-6-ethyl-3,3a,4,5,7,7a-hexahydro-2H-pyrrolo[2,3-c]pyridin-1-yl]-4-(difluoromethyl)pyridazin-3-yl]-5-methyl-phenol (Examples 489 and 490)

Under N₂, to a solution of rac-(3aS,7aR)-1-[6-chloro-5-(difluoromethyl)pyridazin-3-yl]-6-ethyl-3,3a,4,5,7,7a-hexahydro-2H-pyrrolo[2,3-c]pyridine (0.316 mmol, 100 mg) and (2-hydroxy-4-methyl-phenyl)boronic acid (0.474 mmol, 0.0720 g) in 2.5 ml of 1,4-dioxane and 0.5 ml of water were added K₂CO₃ (0.947 mmol, 0.131 g), XPhos (0.00752 g, 1.58e-5 mol) and XPhos Pd G2 (0.0124 g, 1.58e-5 mol). The mixture was purged again with N₂ and stirred at 90° C. for 2 h. After cooling down to rt, the solvent was concentrated under reduced pressure. The residue was purified by flash chromatography on silica gel using a gradient of DCM/MeOH (from 100/0 to 90/10), then repurified by reverse phase preparative LC (spherical C18 25 μm, 12 g YMC-ODS-25, dry loading (Celite®), mobile phase gradient 0.2% aq. NH₄+HCO₃—/MeCN from 75:25 to 40:60) to give 26 mg of a white solid. This solid was purified by SFC (Stationary phase: Chiralpak® IG-3 4.6×100 mm, Mobile phase: CO₂/(iPrOH+0.3% iPrNH₂) 75/25) to provide 2-[6-[(3aR,7aS)-6-ethyl-3,3a,4,5,7,7a-hexahydro-2H-pyrrolo[2,3-c]pyridin-1-yl]-4-(difluoromethyl)pyridazin-3-yl]-5-methyl-phenol, first enantiomer eluting Rt 2.66 min (17 mg, Example 489), as a white solid and 2-[6-[(3aS,7aR)-6-ethyl-3,3a,4,5,7,7a-hexahydro-2H-pyrrolo[2,3-c]pyridin-1-yl]-4-(difluoromethyl)pyridazin-3-yl]-5-methyl-phenol, second enantiomer eluting at Rt=3.25 mn (15 mg, Example 490), as a white solid. The absolute configurations cannot be assigned at this stage.

### Examples 485 and 486: 2-[6-[(3aR,7aS)-6-ethyl-3,3a,4,5,7,7a-hexahydro-2H-pyrrolo[2,3-c]pyridin-1-yl]-4-(difluoromethyl)pyridazin-3-yl]-5-(trifluoromethyl)phenol and 2-[6-[(3aS,7aR)-6-ethyl-3,3a,4,5,7,7a-hexahydro-2H-pyrrolo[2,3-c]pyridin-1-yl]-4-(difluoromethyl)pyridazin-3-yl]-5-(trifluoromethyl)phenol

Under N₂, to a solution of rac-(3aS,7aR)-1-[6-chloro-5-(difluoromethyl)pyridazin-3-yl]-6-ethyl-3,3a,4,5,7,7a-hexahydro-2H-pyrrolo[2,3-c]pyridine (Int 489.2-0.316 mmol, 100 mg) and 2-(4,4,5,5-tetramethyl-1,3,2-dioxaborolan-2-yl)-5-(trifluoromethyl)phenol (0.474 mmol, 0.136 g) in 2.5 ml of 1,4-dioxane and 0.5 ml of water were added K₂CO₃ (0.947 mmol, 0.131 g), XPhos (0.00752 g, 1.58e-5 mol) and XPhos Pd G2 (0.0124 g, 1.58e-5 mol). The mixture was purged again with N₂ and stirred at 90° C. for 2 h. After cooling down to rt, the solvent was concentrated under reduced pressure. The residue was purified by flash chromatography on silica gel using a gradient of DCM/MeOH (from 100/0 to 90/10), then repurified by reverse phase preparative LC (spherical C18 25 μm, 12 g YMC-ODS-25, dry loading (Celite®), mobile phase gradient 0.2% aq. NH₄+HCO₃—/MeCN from 75:25 to 40:60) to give 30 mg of a white solid. This solid was purified by SFC (Stationary phase: Chiralpak® AD-3 4.6×100 mm, Mobile phase: CO₂/(EtOH+0.3% iPrNH₂) 90/10) to provide 2-[6-[(3aR,7aS)-6-ethyl-3,3a,4,5,7,7a-hexahydro-2H-pyrrolo[2,3-c]pyridin-1-yl]-4-(difluoromethyl)pyridazin-3-yl]-5-(trifluoromethyl)phenol, first enantiomer eluting Rt 1.63 min (14 mg, example 485), as a white solid and 2-[6-[(3aS,7aR)-6-ethyl-3,3a,4,5,7,7a-hexahydro-2H-pyrrolo[2,3-c]pyridin-1-yl]-4-(difluoromethyl)pyridazin-3-yl]-5-(trifluoromethyl)phenol, second enantiomer eluting at Rt=2.36 min (14 mg, Example 486), as a white solid. The absolute configurations cannot be assigned at this stage.

### Example 491 and 492: 2-[6-[(3aR,7aS)-6-methyl-3,3a,4,5,7,7a-hexahydro-2H-pyrrolo[2,3-c]pyridin-1-yl]pyridazin-3-yl]-3-ethyl-5-(trifluoromethyl)phenol and 2-[6-[(3aS,7aR)-6-methyl-3,3a,4,5,7,7a-hexahydro-2H-pyrrolo[2,3-c]pyridin-1-yl]pyridazin-3-yl]-3-ethyl-5-(trifluoromethyl)phenol

### Int 491.1: 2-[6-[rac-(3aS,7aR)-6-methyl-3,3a,4,5,7,7a-hexahydro-2H-pyrrolo[2,3-c]pyridin-1-yl]pyridazin-3-yl]-3-ethyl-5-(trifluoromethyl)phenol (Example 452)

In a microwave vial under Ar, to a mixture of 3-ethyl-2-(4,4,5,5-tetramethyl-1,3,2-dioxaborolan-2-yl)-5-(trifluoromethyl)phenol (Int 380.3-188 mg, 0.593 mmol), K₂CO₃ (164 mg, 1.2 mmol) and rac-(3aS,7aR)-1-(6-chloropyridazin-3-yl)-6-methyl-3,3a,4,5,7,7a-hexahydro-2H-pyrrolo[2,3-c]pyridine (Int 342.1-100 mg, 0.396 mmol) in 2 ml of 1,4-dioxane and 0.4 ml of water were added XPhos (9.43 mg, 0.019 mmol) and XPhos Pd G2 (15.6 mg, 0.019 mmol). The mixture was purged again with Ar, then heated at 90° C. for 3 h. After cooling down to rt, the mixture was filtrated and concentrated under reduced pressure. The residue was purified by flash chromatography on silica gel using a gradient of DCM/(MeOH/NH₄OH (10%)) from 100/0 to 80/20) to give the title compound as a yellow oil (123 mg, 76%).

### 2-[6-[(3aR,7aS)-6-methyl-3,3a,4,5,7,7a-hexahydro-2H-pyrrolo[2,3-c]pyridin-1-yl]pyridazin-3-yl]-3-ethyl-5-(trifluoromethyl)phenol and 2-[6-[(3aS,7aR)-6-methyl-3,3a,4,5,7,7a-hexahydro-2H-pyrrolo[2,3-c]pyridin-1-yl]pyridazin-3-yl]-3-ethyl-5-(trifluoromethyl)phenol (Examples 491 and 492)

Both enantiomers of 2-[6-[rac-(3aS,7aR)-6-methyl-3,3a,4,5,7,7a-hexahydro-2H-pyrrolo[2,3-c]pyridin-1-yl]pyridazin-3-yl]-3-ethyl-5-(trifluoromethyl)phenol (Int 491.1-120 mg)) were separated by Supercritical Fluidic Chromatography (SFC—Column Chiralpak® AD-3 4.6×100 mm, Mobile phase: CO₂/(iPrOH+0.3% iPrNH₂) 80/20) to provide 2-[6-[(3aR,7aS)-6-methyl-3,3a,4,5,7,7a-hexahydro-2H-pyrrolo[2,3-c]pyridin-1-yl]pyridazin-3-yl]-3-ethyl-5-(trifluoromethyl)phenol, first enantiomer eluting at Rt=1.87 min (39 mg, Example 491, Enantiomer 1), as a white solid after lyophilization, and 2-[6-[(3aS,7aR)-6-methyl-3,3a,4,5,7,7a-hexahydro-2H-pyrrolo[2,3-c]pyridin-1-yl]pyridazin-3-yl]-3-ethyl-5-(trifluoromethyl)phenol, second enantiomer eluting at Rt=2.70 min (38 mg, Example 492, Enantiomer 2), as a white solid after lyophilization. The absolute configurations cannot be assigned at this stage.

### Examples 493 and 494: 2-[6-[(3aR,7aS)-6-methyl-3,3a,4,5,7,7a-hexahydro-2H-pyrrolo[2,3-c]pyridin-1-yl]pyridazin-3-yl]-5-chloro-3-ethyl-phenol and 2-[6-[(3aS,7aR)-6-methyl-3,3a,4,5,7,7a-hexahydro-2H-pyrrolo[2,3-c]pyridin-1-yl]pyridazin-3-yl]-5-chloro-3-ethyl-phenol

### Int 493.1: 2-[6-[rac-(3aR,7aS)-6-methyl-3,3a,4,5,7,7a-hexahydro-2H-pyrrolo[2,3-c]pyridin-1-yl]pyridazin-3-yl]-5-chloro-3-ethyl-phenol

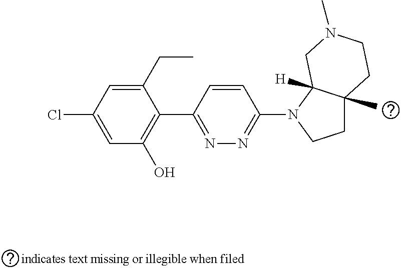 [SMILES: CCc1cc(Cl)cc(O)c1-c1ccc(N2CC[C@@]3(C)CCN(C)C[C@@H]23)nn1]

In a microwave, under Ar, to a solution of 5-chloro-3-ethyl-2-(4,4,5,5-tetramethyl-1,3,2-dioxaborolan-2-yl)phenol ((Int 381.3-55.3 mg, 0.196 mmol), K₂CO₃ (54.1 mg, 0.392 mmol) and rac-(3aS,7aR)-1-(6-chloropyridazin-3-yl)-6-methyl-3,3a,4,5,7,7a-hexahydro-2H-pyrrolo[2,3-c]pyridine (Int 342.1-33.0 mg, 0.131 mol) in 1.2 ml of 1,4-dioxane and 0.3 ml of water were added XPhos (3.11 mg, 0.00653 mmol) and XPhos Pd G2 (5.14 mg, 0.00653 mmol). The mixture was purged again with Ar, then was heated at 90° C. for 3 h. After cooling down to rt, the mixture was filtrated and concentrated under reduced pressure. The residue was purified by flash chromatography on silica gel using a gradient of DCM/(MeOH/NH₄OH (10%)) from 100/0 to 80/20 to give the title compound as a yellow oil (37 mg, 79% yield).

LCMS (Method S): Rt=1.11; MS m/z [M+H]⁺=373.3

### 2-[6-[(3aR,7aS)-6-methyl-3,3a,4,5,7,7a-hexahydro-2H-pyrrolo[2,3-c]pyridin-1-yl]pyridazin-3-yl]-5-chloro-3-ethyl-phenol and 2-[6-[(3aS,7aR)-6-methyl-3,3a,4,5,7,7a-hexahydro-2H-pyrrolo[2,3-c]pyridin-1-yl]pyridazin-3-yl]-5-chloro-3-ethyl-phenol (Examples 493 and 494)

Both enantiomers of 2-[6-[rac-(3aR,7aS)-6-methyl-3,3a,4,5,7,7a-hexahydro-2H-pyrrolo[2,3-c]pyridin-1-yl]pyridazin-3-yl]-5-chloro-3-ethyl-phenol (35 mg) were separated by chiral preparative HPLC (Column Chiralpak® AD-3 4.6×100 mm, Mobile phase: CO₂/(EtOH+0.3% iPrNH₂) 70/30), to provide 2-[6-[(3aR,7aS)-6-methyl-3,3a,4,5,7,7a-hexahydro-2H-pyrrolo[2,3-c]pyridin-1-yl]pyridazin-3-yl]-5-chloro-3-ethyl-phenol, first enantiomer eluting at Rt=1.62 min (15 mg, Example 493), as a white solid and 2-[6-[(3aS,7aR)-6-methyl-3,3a,4,5,7,7a-hexahydro-2H-pyrrolo[2,3-c]pyridin-1-yl]pyridazin-3-yl]-5-chloro-3-ethyl-phenol, second enantiomer eluting at Rt=2.91 min (14 mg, Example 494), as a white solid. The absolute configurations cannot be assigned at this stage.

### Examples 496 and 497: 2-[6-[(3aS,7aR)-6-ethyl-3,3a,4,5,7,7a-hexahydro-2H-pyrrolo[2,3-c]pyridin-1-yl]pyridazin-3-yl]-5-chloro-3-methyl-phenol and 2-[6-[(3aR,7aS)-6-ethyl-3,3a,4,5,7,7a-hexahydro-2H-pyrrolo[2,3-c]pyridin-1-yl]pyridazin-3-yl]-5-chloro-3-methyl-phenol

### Int 496.1: 5-chloro-3-methyl-2-[6-[rac-(3aS,7aR)-6-ethyl-3,3a,4,5,7,7a-hexahydro-2H-pyrrolo[2,3-c]pyridin-1-yl]pyridazin-3-yl]phenol

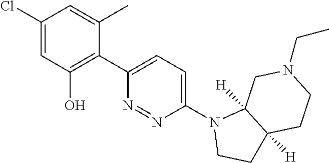 [SMILES: CCN1CC[C@H]2CCN(c3ccc(-c4c(C)cc(Cl)cc4O)nn3)[C@H]2C1]

In a microwave vial, under Ar, to a solution of rac-(3aS,7aR)-1-(6-chloropyridazin-3-yl)-6-ethyl-3,3a,4,5,7,7a-hexahydro-2H-pyrrolo[2,3-c]pyridine (Int 504.1; 0.58 mmol, 155 mg) and (4-chloro-2-hydroxy-6-methylphenyl)boronic acid (0.87 mmol, 145 mg) in 3.2 ml of 1,2-dimethoxyethane are added sodium carbonate (1.45 mmol, 154 mg) and 0.7 ml of water. The solution was purged with Ar, then Pd(PPh₃)₄ (0.04 mmol, 47 mg) was added. The vial was sealed, and the mixture was heated at 100° C. under microwave irradiation for 1 h. The reaction mixture was diluted with EtOAc then filtered and concentrated under reduced pressure. The residue was diluted with CH₂Cl₂, filtered and concentrated under reduced pressure. The residue was purified by flash chromatography on silica gel using DCM/MeOH/ACN (from 100/0/0 to 80/10/10) to give the title compound as a brown solid (130 mg, 63% yield).

LCMS (Method U): Rt=0.87; MS m/z [M+H]⁺=373

### 2-[6-[(3aS,7aR)-6-ethyl-3,3a,4,5,7,7a-hexahydro-2H-pyrrolo[2,3-c]pyridin-1-yl]pyridazin-3-yl]-5-chloro-3-methyl-phenol and 2-[6-[(3aR,7aS)-6-ethyl-3,3a,4,5,7,7a-hexahydro-2H-pyrrolo[2,3-c]pyridin-1-yl]pyridazin-3-yl]-5-chloro-3-methyl-phenol (Examples 496 and 497)

Both enantiomers of 5-chloro-3-methyl-2-[6-[rac-(3aS,7aR)-6-ethyl-3,3a,4,5,7,7a-hexahydro-2H-pyrrolo[2,3-c]pyridin-1-yl]pyridazin-3-yl]phenol (126 mg) were separated by chiral preparative HPLC (Column Chiralpak® IG 5 μm, 250×30 mm, Flow rate 45 ml/min, eluent (n-heptane/ethanol 85/15)+0.1% TEA), to provide 2-[6-[(3aS,7aR)-6-ethyl-3,3a,4,5,7,7a-hexahydro-2H-pyrrolo[2,3-c]pyridin-1-yl]pyridazin-3-yl]-5-chloro-3-methyl-phenol, first enantiomer eluting at Rt=20.7 min (28 mg, Example 496), as a white solid and 2-[6-[(3aR,7aS)-6-ethyl-3,3a,4,5,7,7a-hexahydro-2H-pyrrolo[2,3-c]pyridin-1-yl]pyridazin-3-yl]-5-chloro-3-methyl-phenol, second enantiomer eluting at Rt=24.5 min (30 mg, Example 497), as a white solid. The absolute configurations cannot be assigned at this stage.

### Examples 498 and 499: 2-[6-[(3aS,7aR)-6-ethyl-3,3a,4,5,7,7a-hexahydro-2H-pyrrolo[2,3-c]pyridin-1-yl]pyridazin-3-yl]-3-methyl-phenol and 2-[6-[(3aR,7aS)-6-ethyl-3,3a,4,5,7,7a-hexahydro-2H-pyrrolo[2,3-c]pyridin-1-yl]pyridazin-3-yl]-3-methyl-phenol

### Int 498.1: 3-methyl-2-[6-[rac-(3aS,7aR)-6-ethyl-3,3a,4,5,7,7a-hexahydro-2H-pyrrolo[2,3-c]pyridin-1-yl]pyridazin-3-yl]phenol

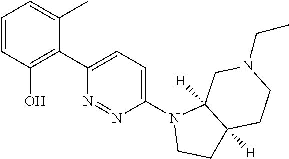 [SMILES: CCN1CC[C@H]2CCN(c3ccc(-c4c(C)cccc4O)nn3)[C@H]2C1]

In a microwave vial, under Ar, to a solution of rac-(3aS,7aR)-1-(6-chloropyridazin-3-yl)-6-ethyl-3,3a,4,5,7,7a-hexahydro-2H-pyrrolo[2,3-c]pyridine (Int 504.1; 0.56 mmol, 151 mg) and (2-hydroxy-6-methylphenyl)boronic acid (0.85 mmol, 129 mg) in 3.2 ml of 1,2-dimethoxyethane were added sodium carbonate (1.42 mmol, 150 mg) and 0.7 ml of water. The solution was purged with Ar for 3 min, then Pd(PPh₃)₄ (0.04 mmol, 46 mg) was added.

The vial was sealed, and the mixture was heated at 100° C. under microwave irradiation for 1 h. The reaction mixture was diluted with EtOAc, filtered on hydrophobic column and concentrated under reduce pressure. The residue was diluted with CH₂Cl₂, filtered and concentrated under reduce pressure. The residue was purified by flash chromatography on silica gel using DCM/MeOH/ACN (from 100/0/0 to 80/10/10) to give the title compound as a brown solid (110 mg, 57% yield).

LCMS (Method U): Rt=0.78; MS m/z [M+H]⁺=353

### 2-[6-[(3aS,7aR)-6-ethyl-3,3a,4,5,7,7a-hexahydro-2H-pyrrolo[2,3-c]pyridin-1-yl]pyridazin-3-yl]-3-methyl-phenol and 2-[6-[(3aR,7aS)-6-ethyl-3,3a,4,5,7,7a-hexahydro-2H-pyrrolo[2,3-c]pyridin-1-yl]pyridazin-3-yl]-3-methyl-phenol (Examples 498 and 499)

Both enantiomers of 3-methyl-2-[6-[rac-(3aS,7aR)-6-ethyl-3,3a,4,5,7,7a-hexahydro-2H-pyrrolo[2,3-c]pyridin-1-yl]pyridazin-3-yl]phenol (108 mg) were separated by chiral preparative HPLC (Column Chiralpak® IG 5 μm, 250×30 mm, Flow rate 115 ml/min, eluent (CO₂/EtOH+0.1% TEA 65/35), to provide 2-[6-[(3aS,7aR)-6-ethyl-3,3a,4,5,7,7a-hexahydro-2H-pyrrolo[2,3-c]pyridin-1-yl]pyridazin-3-yl]-3-methyl-phenol, first enantiomer eluting at Rt=8.9 min (36 mg, Example 498), as a white solid and 2-[6-[(3aR,7aS)-6-ethyl-3,3a,4,5,7,7a-hexahydro-2H-pyrrolo[2,3-c]pyridin-1-yl]pyridazin-3-yl]-3-methyl-phenol, second enantiomer eluting at Rt=11.1 min (32 mg, Example 499), as a white solid. The absolute configurations cannot be assigned at this stage.

### Examples 500 and 501: 2-[6-[(3aS,7aR)-6-methyl-3,3a,4,5,7,7a-hexahydro-2H-pyrrolo[2,3-c]pyridin-1-yl]-4-(difluoromethyl)pyridazin-3-yl]-5-chloro-phenol and 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-chloro-phenol

### Int 500.1: 5-chloro-2-[4-(difluoromethyl)-6-[rac-(3aS,7aR)-6-methyl-3,3a,4,5,7,7a-hexahydro-2H-pyrrolo[2,3-c]pyridin-1-yl]pyridazin-3-yl]phenol

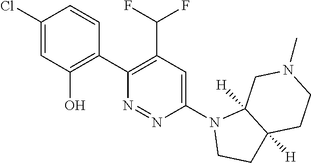 [SMILES: CN1CC[C@H]2CCN(c3cc(C(F)F)c(-c4ccc(Cl)cc4O)nn3)[C@H]2C1]

In a microwave vial, under Ar, to a solution of rac-(3aS,7aR)-1-[6-chloro-5-(difluoromethyl)pyridazin-3-yl]-6-methyl-3,3a,4,5,7,7a-hexahydro-2H-pyrrolo[2,3-c]pyridine (Int 506.3; 0.27 mmol, 82 mg) and (4-chloro-2-hydroxy-phenyl)boronic acid (0.41 mmol, 70 mg) in 1.3 ml of 1,2-dimethoxyethane were added sodium carbonate (0.68 mmol, 72 mg) and 0.35 ml of water. The solution was purged with Ar, then Pd(PPh₃)₄ (0.02 mmol, 22 mg) was added. The vial was sealed, and the mixture was heated at 100° C. under microwave irradiation for 1 h. The reaction mixture was diluted with EtOAc, filtered and concentrated under reduce pressure. The residue was diluted with CH₂Cl₂, filtered and concentrated under reduce pressure. The crude product was purified by flash chromatography on silica gel using DCM/MeOH/ACN (from 100/0/0 to 84/8/8) to give the title compound as a pale yellow solid (65 mg, 61% yield).

LCMS (Method U): Rt=0.99; MS m/z [M+H]⁺=395

### 2-[6-[(3aS,7aR)-6-methyl-3,3a,4,5,7,7a-hexahydro-2H-pyrrolo[2,3-c]pyridin-1-yl]-4-(difluoromethyl)pyridazin-3-yl]-5-chloro-phenol and 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-chloro-phenol (Examples 500 et 501)

Both enantiomers of 5-chloro-2-[4-(difluoromethyl)-6-[rac-(3aS,7aR)-6-methyl-3,3a,4,5,7,7a-hexahydro-2H-pyrrolo[2,3-c]pyridin-1-yl]pyridazin-3-yl]phenol (63 mg) were separated by chiral preparative HPLC (Column Chiralpak® AD-H 5 μm, 250×30 mm, Flow rate 100 ml/min, eluent (CO₂/ethanol+0.1% TEA 80/20), to provide 2-[6-[(3aS,7aR)-6-methyl-3,3a,4,5,7,7a-hexahydro-2H-pyrrolo[2,3-c]pyridin-1-yl]-4-(difluoromethyl) pyridazin-3-yl]-5-chloro-phenol, first enantiomer eluting Rt=4.7 min (23 mg, Example 500), as a white solid and 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-chloro-phenol, second enantiomer eluting at Rt=7.2 min (20 mg, Example 501), as a white solid. The absolute configurations cannot be assigned at this stage.

### Examples 502 and 503: 2-[6-[(3aS,7aR)-6-methyl-3,3a,4,5,7,7a-hexahydro-2H-pyrrolo[2,3-c]pyridin-1-yl]pyridazin-3-yl]-5-chloro-3-methyl-phenol and 2-[6-[(3aR,7aS)-6-methyl-3,3a,4,5,7,7a-hexahydro-2H-pyrrolo[2,3-c]pyridin-1-yl]pyridazin-3-yl]-5-chloro-3-methyl-phenol

### Int 502.1: tert-butyl rac-(3aR,7aR)-1-(6-chloropyridazin-3-yl)-3,3a,4,5,7,7a-hexahydro-2H-pyrrolo[2,3-c]pyridine-6-carboxylate

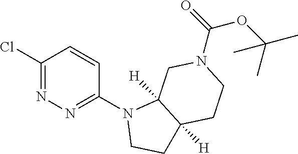 [SMILES: CC(C)(C)OC(=O)N1CC[C@H]2CCN(c3ccc(Cl)nn3)[C@H]2C1]

In a microwave vial, under Ar, to a suspension of tert-butyl rac-(3aR,7aR)-1,2,3,3a,4,5,7,7a-octahydropyrrolo[2,3-c]pyridine-6-carboxylate (0.98 mmol, 222 mg) and 3,6-dichloropyridazine (0.89 mmol, 133 mg) in 2.66 ml of n-butanol was added triethylamine (1.96 mmol, 0.27 ml). The reaction mixture was heated at 130° C. under microwave irradiation for 3 h30. In another microwave vial, under Ar, to a suspension of tert-butyl rac-(3aR,7aR)-1,2,3,3a,4,5,7,7a-octahydropyrrolo[2,3-c]pyridine-6-carboxylate (5.25 mmol, 1.19 g) and 3,6-dichloropyridazine (4.77 mmol, 711 mg) in 14.2 ml of n-butanol was added triethylamine (10.50 mmol, 1.46 ml). The reaction mixture was heated at 130° C. under microwave irradiation for 3 h30.

The reaction mixtures were combined and concentrated under reduced pressure. The residue was dissolved in EtOAc and washed with water (3 times), dried over Na₂SO₄, filtered and concentrated under reduced pressure. The residue was purified by flash chromatography on silica gel using DCM/MeOH/ACN (from 100/0/0 to 95/2.5/2.5) to give the title compound as a beige solid (1.35 g, 71% yield).

LCMS (Method U): Rt=1.54; MS m/z [M+H]⁺=339

### Int 502.2: rac-(3aS,7aR)-1-(6-chloropyridazin-3-yl)-2,3,3a,4,5,6,7,7a-octahydropyrrolo[2,3-c]pyridine;hydrochloride

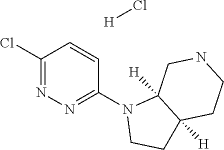

To a solution of tert-butyl rac-(3aR,7aR)-1-(6-chloropyridazin-3-yl)-3,3a,4,5,7,7a-hexahydro-2H-pyrrolo[2,3-c]pyridine-6-carboxylate (3.98 mmol, 1.35 g) in 8 ml of dichloromethane was added 4N hydrochloric acid in dioxan (59.8 mmol, 14.9 ml). The reaction mixture was stirred at rt overnight, then concentrated under reduced pressure. MeOH was added and the mixture was concentrated under reduced pressure to give the title compound (1.5 g) as a brown solid, which was used without further purification in the next step.

LCMS (Method U): Rt=0.19 and 0.29; MS m/z [M+H]⁺=239

### Int 502.3: rac-(3aS,7aR)-1-(6-chloropyridazin-3-yl)-6-methyl-3,3a,4,5,7,7a-hexahydro-2H-pyrrolo[2,3-c]pyridine

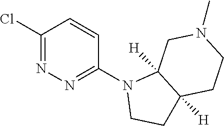 [SMILES: CN1CC[C@H]2CCN(c3ccc(Cl)nn3)[C@H]2C1]

To a solution of rac-(3aS,7aR)-1-(6-chloropyridazin-3-yl)-2,3,3a,4,5,6,7,7a-octahydropyrrolo[2,3-c]pyridine;hydrochloride (0.73 mmol, 200 mg) in 7 ml of DCM and 1.6 ml of MeOH was added 37% aqueous solution of formaldehyde (1.82 mmol, 135 μl). The reaction mixture was stirred at room temperature for 10 minutes, then sodium triacetoxyborohydride (2.91 mmol, 616 mg) was added. The reaction mixture was stirred at room temperature for 1 h. 2 ml of a saturated aqueous sodium bicarbonate solution were added, and the organic layer was concentrated under reduced pressure. The aqueous layer was extracted 3 times with DCM and the combined organic layers were filtered and concentrated under reduced pressure. The residue was purified by flash chromatography on silica gel using a gradient of DCM/MeOH/ACN (from 100/0/0 to 80/10/10) to give the title compound as a pale yellow solid (176 mg, 96% yield).

LCMS (Method U): Rt=0.17 and 0.29; MS m/z [M+H]⁺=253

### Int 502.4: 5-chloro-3-methyl-2-[6-[rac-(3aS,7aR)-6-methyl-3,3a,4,5,7,7a-hexahydro-2H-pyrrolo[2,3-c]pyridin-1-yl]pyridazin-3-yl]phenol

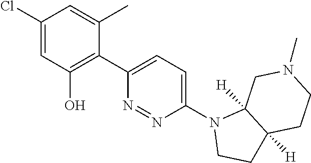 [SMILES: Cc1cc(Cl)cc(O)c1-c1ccc(N2CC[C@@H]3CCN(C)C[C@@H]32)nn1]

In a microwave vial, under Ar, to a solution of rac-(3aS,7aR)-1-(6-chloropyridazin-3-yl)-6-methyl-3,3a,4,5,7,7a-hexahydro-2H-pyrrolo[2,3-c]pyridine (0.69 mmol, 174 mg) and (4-chloro-2-hydroxy-6-methylphenyl)boronic acid (1.03 mmol, 192 mg) in 3.2 ml of 1,2-dimethoxyethane were added sodium carbonate (1.72 mmol, 182 mg) and 0.7 ml of water. The solution was purged with Ar, then Pd(PPh₃)₄ (0.04 mmol, 48 mg) was added. The vial was sealed, and the mixture was heated at 100° C. under microwave irradiation for 1 h. The reaction mixture was diluted with EtOAc then filtered and concentrated under reduced pressure. The residue was diluted with CH₂Cl₂, filtered and concentrated under reduced pressure. The residue was purified by flash chromatography on silica gel using DCM/MeOH/ACN (from 100/0/0 to 80/10/10) to give the title compound as a pale yellow solid (146 mg, 59% yield).

LCMS (Method U): Rt=0.85; MS m/z [M+H]⁺=359

### 2-[6-[(3aS,7aR)-6-methyl-3,3a,4,5,7,7a-hexahydro-2H-pyrrolo[2,3-c]pyridin-1-yl]pyridazin-3-yl]-5-chloro-3-methyl-phenol and 2-[6-[(3aR,7aS)-6-methyl-3,3a,4,5,7,7a-hexahydro-2H-pyrrolo[2,3-c]pyridin-1-yl]pyridazin-3-yl]-5-chloro-3-methyl-phenol (Examples 502 and 503)

Both enantiomers of 5-chloro-3-methyl-2-[6-[rac-(3aS,7aR)-6-methyl-3,3a,4,5,7,7a-hexahydro-2H-pyrrolo[2,3-c]pyridin-1-yl]pyridazin-3-yl]phenol (144 mg) were separated by chiral preparative HPLC (Column: chiral phase LC (Column Chiralpak® AD-H 20 μm, 350×76.5 mm, Flow rate 400 ml/min, eluent (n-heptane+0.1% TEA/ethanol+0.1% TEA 90/10 to 50/50), to provide 2-[6-[(3aS,7aR)-6-methyl-3,3a,4,5,7,7a-hexahydro-2H-pyrrolo[2,3-c]pyridin-1-yl]pyridazin-3-yl]-5-chloro-3-methyl-phenol, first enantiomer eluting at Rt=33.4 min (30 mg, example 502), as a white solid and 2-[6-[(3aR,7aS)-6-methyl-3,3a,4,5,7,7a-hexahydro-2H-pyrrolo[2,3-c]pyridin-1-yl]pyridazin-3-yl]-5-chloro-3-methyl-phenol, second enantiomer eluting at Rt=40.3 min (30 mg, Example 503), as a white solid. The absolute configurations cannot be assigned at this stage.

### Examples 504 and 505: 2-[6-[(3aS,7aR)-6-ethyl-3,3a,4,5,7,7a-hexahydro-2H-pyrrolo[2,3-c]pyridin-1-yl]pyridazin-3-yl]-3,5-dimethyl-phenol and 2-[6-[(3aR,7aS)-6-ethyl-3,3a,4,5,7,7a-hexahydro-2H-pyrrolo[2,3-c]pyridin-1-yl]pyridazin-3-yl]-3,5-dimethyl-phenol

Int 504.1: rac-(3aS,7aR)-1-(6-chloropyridazin-3-yl)-6-ethyl-3,3a,4,5,7,7a-hexahydro-2H-pyrrolo[2,3-c]pyridine

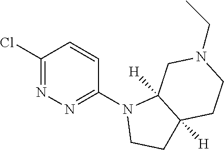 [SMILES: CCN1CC[C@H]2CCN(c3ccc(Cl)nn3)[C@H]2C1]

At 0° C., to a solution of rac-(3aS,7aR)-1-(6-chloropyridazin-3-yl)-2,3,3a,4,5,6,7,7a-octahydropyrrolo[2,3-c]pyridine;hydrochloride (Int 502.2; 2.19 mmol, 604 mg) in 16 ml of MeOH was added acetaldehyde (110 mmol, 6.16 ml) previously cooled at 0° C. After stirring 1 min at 0° C., the reaction mixture was stirred at room temperature for 10 minutes. Sodium triacetoxyborohydride (8.78 mmol, 1.86 g) was then added and the reaction mixture was stirred at room temperature for 1 h. 13.5 ml of a saturated aqueous sodium bicarbonate solution was added, and the organic layer was concentrated under reduced pressure. The aqueous layer was extracted 3 times with DCM and the combined organic layers were filtered and concentrated under reduced pressure. The residue was purified by flash chromatography on silica gel using DCM/MeOH/ACN (from 100/0/0 to 80/10/10) to give the title compound as a beige solid (512 mg, 87% yield).

LCMS (Method U): Rt=0.72; MS m/z [M+H]⁺=267

### Int 504.2: 3,5-dimethyl-2-[6-[rac-(3aS,7aR)-6-ethyl-3,3a,4,5,7,7a-hexahydro-2H-pyrrolo[2,3-c]pyridin-1-yl]pyridazin-3-yl]phenol

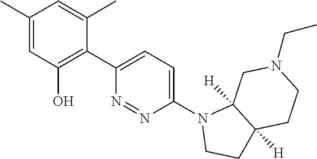 [SMILES: CCN1CC[C@H]2CCN(c3ccc(-c4c(C)cc(C)cc4O)nn3)[C@H]2C1]

In a microwave vial, under Ar, to a solution of rac-(3aS,7aR)-1-(6-chloropyridazin-3-yl)-6-ethyl-3,3a,4,5,7,7a-hexahydro-2H-pyrrolo[2,3-c]pyridine (0.58 mmol, 155 mg) and (2-hydroxy-4,6-dimethylphenyl)boronic acid (0.87 mmol, 145 mg) in 3.2 ml of 1,2-dimethoxyethane were added sodium carbonate (1.45 mmol, 154 mg) and 0.7 ml of water.

The solution was purged with Ar, then Pd(PPh₃)₄ (0.04 mmol, 47 mg) was added. The vial was sealed, and the mixture was heated at 100° C. under microwave irradiation for 1 h. The reaction mixture was diluted with EtOAc then filtered and concentrated under reduced pressure. The residue was diluted with CH₂Cl₂, filtered and concentrated under reduced pressure. The residue was purified by flash chromatography on silica gel using DCM/MeOH/ACN (from 100/0/0 to 80/10/10) to give the title compound as a brown solid (130 mg, 63% yield).

LCMS (Method U): Rt=0.77; MS m/z [M+H]⁺=267

### 2-[6-[(3aS,7aR)-6-ethyl-3,3a,4,5,7,7a-hexahydro-2H-pyrrolo[2,3-c]pyridin-1-yl]pyridazin-3-yl]-3,5-dimethyl-phenol and 2-[6-[(3aR,7aS)-6-ethyl-3,3a,4,5,7,7a-hexahydro-2H-pyrrolo[2,3-c]pyridin-1-yl]pyridazin-3-yl]-3,5-dimethyl-phenol (Examples 504 et 505)

Both enantiomers of 3,5-dimethyl-2-[6-[rac-(3aS,7aR)-6-ethyl-3,3a,4,5,7,7a-hexahydro-2H-pyrrolo[2,3-c]pyridin-1-yl]pyridazin-3-yl]phenol (125 mg) were separated by chiral preparative HPLC (Column Chiralpak® AY-H 5 μm, 250×30 mm, Flow rate 100 ml/min, eluent (CO₂/EtOH+0.1% TEA 75/25), to provide 2-[6-[(3aS,7aR)-6-ethyl-3,3a,4,5,7,7a-hexahydro-2H-pyrrolo[2,3-c]pyridin-1-yl]pyridazin-3-yl]-3,5-dimethyl-phenol, first enantiomer eluting at Rt=15 min (42 mg, Example 504), as a white solid and 2-[6-[(3aR,7aS)-6-ethyl-3,3a,4,5,7,7a-hexahydro-2H-pyrrolo[2,3-c]pyridin-1-yl]pyridazin-3-yl]-3,5-dimethyl-phenol, second enantiomer eluting at Rt=19.1 min (39 mg, Example 505), as a white solid. The absolute configurations cannot be assigned at this stage.

### Examples 506 and 507: 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-methyl-phenol and 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-methyl-phenol

### Int 506.1 and 506.1bis: tert-butyl rac-(3aR,7aR)-1-[6-chloro-5-(difluoromethyl)pyridazin-3-yl]-3,3a,4,5,7,7a-hexahydro-2H-pyrrolo[2,3-c]pyridine-6-carboxylate (Int 506.1) and tert-butyl rac-(3aR,7aR)-1-[6-chloro-4-(difluoromethyl)pyridazin-3-yl]-3,3a,4,5,7,7a-hexahydro-2H-pyrrolo[2,3-c]pyridine-6-carboxylate (Int 506.1bis)

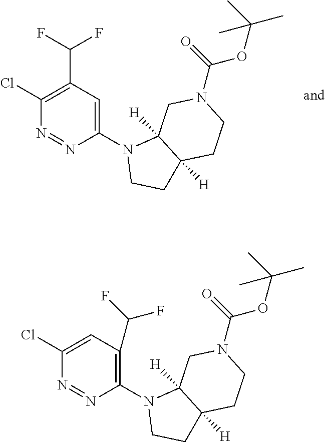 [SMILES: CC(C)(C)OC(=O)N1CC[C@H]2CCN(c3cc(C(F)F)c(Cl)nn3)[C@H]2C1 ; CC(C)(C)OC(=O)N1CC[C@H]2CCN(c3nnc(Cl)cc3C(F)F)[C@H]2C1]

In a microwave vial, under Ar, to a solution of tert-butyl rac-(3aR,7aR)-1,2,3,3a,4,5,7,7a-octahydropyrrolo[2,3-c]pyridine-6-carboxylate (2.65 mmol, 600 mg) and 3,6-dichloro-4-(difluoromethyl)pyridazine (3.45 mmol, 686 mg) in 20 ml of DMSO was added potassium phosphate tribasic (6.63 mmol, 1.48 g) and the reaction was heated at 90° C. under microwave irradiation for 2 h. The reaction mixture was taken up in 400 ml of diethyl ether. The organic layer was washed twice with water, dried over Na₂SO₄, filtered and concentrated under reduce pressure. The residue was purified by flash chromatography on silica gel using DCM/EtOAc (from 100/0 to 80/20) to give tert-butyl rac-(3aR,7aR)-1-[6-chloro-5-(difluoromethyl)pyridazin-3-yl]-3,3a,4,5,7,7a-hexahydro-2H-pyrrolo[2,3-c]pyridine-6-carboxylate (Int 506.1) as a pale yellow solid (363 mg, 35% yield) and tert-butyl rac-(3aR,7aR)-1-[6-chloro-4-(difluoromethyl)pyridazin-3-yl]-3,3a,4,5,7,7a-hexahydro-2H-pyrrolo[2,3-c]pyridine-6-carboxylate (Int 506.1bis) as a pale yellow solid (206 mg, 20% yield).

Int 506.1—LCMS: (Method U): Rt=1.72; MS m/z [M+H]⁺=389

Int 506.1bis—LCMS: (Method U): Rt=1.73; MS m/z [M+H]⁺=389

### Int 506.2: rac-(3aS,7aR)-1-[6-chloro-5-(difluoromethyl)pyridazin-3-yl]-2,3,3a,4,5,6,7,7a-octahydropyrrolo[2,3-c]pyridine;hydrochloride

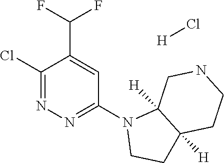

To a solution of tert-butyl rac-(3aR,7aR)-1-[6-chloro-5-(difluoromethyl)pyridazin-3-yl]-3,3a,4,5,7,7a-hexahydro-2H-pyrrolo[2,3-c]pyridine-6-carboxylate (1.03 mmol, 402 mg) in 3 ml of dichloromethane was added 4N hydrochloric acid in dioxan (15.51 mmol, 3.88 ml). The reaction mixture was stirred at rt for 40 h, then concentrated under reduced pressure. MeOH and toluene were added, and the mixture was concentrated under reduced pressure to give the title compound (492 mg) as a brown oil, which was used without further purification in the next step.

LCMS (Method U): Rt=0.67; MS m/z [M+H]⁺=289

### Int 506.3: rac-(3aS,7aR)-1-[6-chloro-5-(difluoromethyl)pyridazin-3-yl]-6-methyl-3,3a,4,5,7,7a-hexahydro-2H-pyrrolo[2,3-c]pyridine

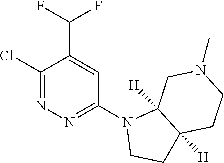 [SMILES: CN1CC[C@H]2CCN(c3cc(C(F)F)c(Cl)nn3)[C@H]2C1]

To a solution of rac-(3aS,7aR)-1-[6-chloro-5-(difluoromethyl)pyridazin-3-yl]-2,3,3a,4,5,6,7,7a-octahydropyrrolo[2,3-c]pyridine;hydrochloride (1.03 mmol, 336 mg) in 12 ml of DCM and 1 ml of MeOH was added 37% aqueous solution of formaldehyde (2.58 mmol, 192 μl). The reaction mixture was stirred at room temperature for 10 minutes, then sodium triacetoxyborohydride (4.13 mmol, 876 mg) was added. The reaction mixture was stirred at room temperature for 1 h. 3 ml of a saturated aqueous sodium bicarbonate solution was added, and the reaction mixture was diluted with CH₂Cl₂. The aqueous layer was extracted twice with DCM, and the combined organic layers were filtered and concentrated under reduced pressure. The residue was purified by flash chromatography on silica gel using a gradient of DCM/MeOH/ACN (from 100/0/0 to 86/7/7) to give the title compound as a pale yellow solid (331 mg, quantitative yield).

LCMS (Method U): Rt=0.69; MS m/z [M+H]⁺=303

### Int 506.4: 2-[4-(difluoromethyl)-6-[rac-(3aS,7aR)-6-methyl-3,3a,4,5,7,7a-hexahydro-2H-pyrrolo[2,3-c]pyridin-1-yl]pyridazin-3-yl]-5-methyl-phenol

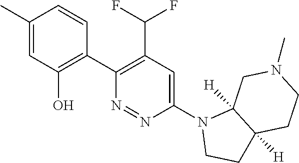 [SMILES: Cc1ccc(-c2nnc(N3CC[C@@H]4CCN(C)C[C@@H]43)cc2C(F)F)c(O)c1]

In a microwave vial, under Ar, to a solution of rac-(3aS,7aR)-1-[6-chloro-5-(difluoromethyl)pyridazin-3-yl]-6-methyl-3,3a,4,5,7,7a-hexahydro-2H-pyrrolo[2,3-c]pyridine (0.27 mmol, 81 mg) and 5-methyl-2-(4,4,5,5-tetramethyl-1,3,2-dioxaborolan-2-yl)phenol (0.40 mmol, 94 mg) in 1.3 ml of 1,2-dimethoxyethane were added sodium carbonate (0.67 mmol, 71 mg) and 0.35 ml of water. The solution was purged with Ar for 3 min, then Pd(PPh₃)₄ (0.02 mmol, 22 mg) was added. The vial was sealed, and the mixture was heated at 100° C. under microwave irradiation for 1 h. The reaction mixture was diluted with EtOAc, filtered and concentrated under reduce pressure. The residue was diluted with CH₂Cl₂, filtered on hydrophobic column and concentrated under reduce pressure. The crude product was purified by flash chromatography on silica gel using DCM/MeOH/ACN (from 100/0/0 to 84/8/8) to give the title compound as a pale yellow solid (76 mg, 76% yield).

LCMS (Method U): Rt=0.94; MS m/z [M+H]⁺=375

### 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-methyl-phenol and 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-methyl-phenol (Examples 506 and 507)

Both enantiomers of 2-[4-(difluoromethyl)-6-[rac-(3aS,7aR)-6-methyl-3,3a,4,5,7,7a-hexahydro-2H-pyrrolo[2,3-c]pyridin-1-yl]pyridazin-3-yl]-5-methyl-phenol (74 mg) were separated by chiral preparative HPLC (Column Chiralpak® AD-H 5 μm, 250×30 mm, Flow rate 105 ml/min, eluent (CO₂/EtOH+0.1% TEA 85/15), to provide 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-methyl-phenol, first enantiomer eluting Rt=8.15 min (29 mg, Example 506), as a white solid and 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-methyl-phenol, second enantiomer eluting at Rt=11.9 min (28 mg, Example 507), as a white solid. The absolute configurations cannot be assigned at this stage.

### Example 508: 5-ethynyl-2-[6-[(2S)-2-(hydroxymethyl)morpholin-4-yl]pyridazin-3-yl]-3-methyl-phenol

### Int 508.4: 5-[2-[tert-butyl(dimethyl)silyl]ethynyl]-2-[6-[(2S)-2-(hydroxymethyl) morpholin-4-yl]pyridazin-3-yl]-3-methyl-phenol

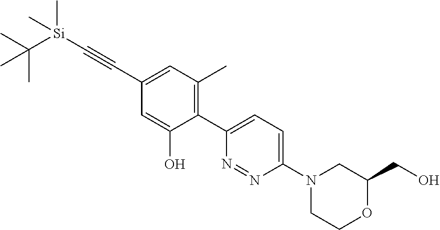 [SMILES: Cc1cc(C#C[Si](C)(C)C(C)(C)C)cc(O)c1-c1ccc(N2CCO[C@H](CO)C2)nn1]

At 0° C., under N₂, In a sealed tube, XPhos Pd G2 (6.87 mg, 8.73e-6 mol) and XPhos (4.16 mg, 8.73e-6 mol) were added to a solution of 5-[2-[tert-butyl(dimethyl)silyl]ethynyl]-3-methyl-2-(4,4,5,5-tetramethyl-1,3,2-dioxaborolan-2-yl)phenol (Int 508.3-65.0 mg, 0.175 mmol), [(2S)-4-(6-chloropyridazin-3-yl)morpholin-2-yl]methanol (Int 8.1-40.1 mg, 0.175 mmol) and K₂CO₃ (48.2 mg, 0.349 mmol) in 1 ml of 1,4-dioxane and 0.25 ml of water. The mixture was stirred at 95° C. for 2 h. After cooling down to rt, the mixture was filtered and concentrated under reduced pressure. The residue was purified by flash chromatography on silica gel using DCM/MeOH (from 100/0 to 97/3, then isocratic) to give the title compound as a white solid (44 mg, 60% pure, 40% yield).

LCMS (Method R): Rt=1.53; MS m/z [M+H]+=440

### 5-ethynyl-2-[6-[(2S)-2-(hydroxymethyl)morpholin-4-yl]pyridazin-3-yl]-3-methyl-phenol (Example 508)

To a solution of 5-[2-[tert-butyl(dimethyl)silyl]ethynyl]-2-[6-[(2S)-2-(hydroxymethyl) morpholin-4-yl]pyridazin-3-yl]-3-methyl-phenol (60.0%, 44.0 mg, 6.01e-5 mol) in 0.8 ml of THF was added TBAF 1 M in THF (1.00 mol/L, 90.1 mL, 9.01e-5 mol). The solution was stirred at rt for 1 h. The mixture was diluted with EtOAc and water was added. The aqueous layer was separated and extracted with EtOAc. The combined organic layers were dried over MgSO₄, filtered and concentrated under reduced pressure. The residue was purified by flash chromatography on silica gel using Heptane/EtOA (from 90/10 to 20/80, then isocratic), the repurified by reverse phase preparative LC (spherical C18 25 μm, 25 g YMC-ODS-25, dry loading (Celite®), mobile phase gradient 0.2% aq. NH₄⁺HCO₃⁻/MeCN from 90/10 to 50/50) to give the title compound as a white solid (15 mg, 77%).

### Example 509: 2-[6-[(2S)-2-(hydroxymethyl)morpholin-4-yl]pyridazin-3-yl]-5-methyl-3-(trifluoromethyl)phenol

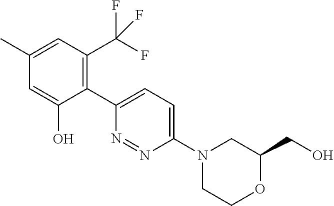 [SMILES: Cc1cc(O)c(-c2ccc(N3CCO[C@H](CO)C3)nn2)c(C(F)(F)F)c1]

In a microwave vial, under N₂, a solution of 5-methyl-2-(4,4,5,5-tetramethyl-1,3,2-dioxaborolan-2-yl)-3-(trifluoromethyl)phenol (Int 509.2-0.200 g, 0.666 mmol), [(2S)-4-(6-chloropyridazin-3-yl)morpholin-2-yl]methanol (Int 8.1-0.102 g, 0.444 mol) and K₂CO₃ (0.123 g, 0.888 mmol) in 2.7 ml of 1,4-dioxane and 0.65 ml of water, were added XPhos Pd G2 (0.0175 g, 2.22e-5 mol) and XPhos (0.0106 g, 2.22e-5 mol). The mixture was stirred at 95° C. for 2 h. After cooling down to rt, the mixture was filtered and concentrated under reduced pressure. The residue was purified by flash chromatography on silica gel using DCM/MeOH (from 100/0 to 95/5 then 90/10), then was purified again by flash chromatography on silica gel (column Spherical SiOH 15 μm) using DCM/MeOH (from 100/0 to 95/5 then 90/10), to give the title compound as a white solid (25 mg, 39%).

### Example 510 and 511: 1-[(3aS,7aS)-1-[5-(difluoromethyl)-6-[2-hydroxy-4-(trifluoromethyl)phenyl]pyridazin-3-yl]-3,3a,4,5,7,7a-hexahydro-2H-pyrrolo[2,3-c]pyridin-6-yl]ethenone and 1-[(3aR,7aR)-1-[5-(difluoromethyl)-6-[2-hydroxy-4-(trifluoromethyl)phenyl]pyridazin-3-yl]-3,3a,4,5,7,7a-hexahydro-2H-pyrrolo[2,3-c]pyridin-6-yl]ethenone

### Int 510.1: rac-(3aS,7aR)-1-[6-chloro-5-(difluoromethyl)pyridazin-3-yl]-2,3,3a,4,5,6,7,7a-octahydropyrrolo[2,3-c]pyridine;2,2,2-trifluoroacetic acid

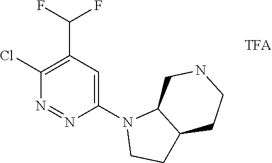 [SMILES: FC(F)c1cc(N2CC[C@@H]3CCNC[C@@H]32)nnc1Cl]

At 0° C., to a solution of tert-butyl rac-(3aR,7aR)-1-[6-chloro-5-(difluoromethyl)pyridazin-3-yl]-3,3a,4,5,7,7a-hexahydro-2H-pyrrolo[2,3-c]pyridine-6-carboxylate (Int 487.1-260 mg, 0.000669 mol) in 5 ml of DCM was added TFA (0.762 g, 0.00669 mol) and the solution was stirred at rt for 18 h. The mixture was concentrated under reduced pressure to give the title compound as a brown oil (330 mg) which was used without further purification in the next step.

LCMS (Method S): Rt=0.84; [M+H]+=289

### Int 510.2: 1-[rac-(3aR,7aR)-1-[6-chloro-5-(difluoromethyl)pyridazin-3-yl]-3,3a,4,5,7,7a-hexahydro-2H-pyrrolo[2,3-c]pyridin-6-yl]ethanone

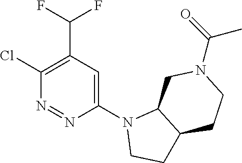 [SMILES: CC(=O)N1CC[C@H]2CCN(c3cc(C(F)F)c(Cl)nn3)[C@H]2C1]

At 0° C., to a solution of rac-(3aS,7aR)-1-[6-chloro-5-(difluoromethyl)pyridazin-3-yl]-2,3,3a,4,5,6,7,7a-octahydropyrrolo[2,3-c]pyridine;2,2,2-trifluoroacetic acid (100 mg, 0.000248 mol) and DIPEA (0.128 g, 0.000993 mol) in 2 ml of DCM was added acetyl chloride (0.0390 g, 0.000497 mol) and the resulting mixture was stirred at rt for 18 h. The mixture was concentrated under reduced pressure. The residue was purified by flash chromatography on silica gel using a gradient DCM/MeOH (100/0 to 90/10) to give the title compound as a brown solid (91 mg, 90%).

LCMS (Method S): Rt=1.04; MS m/z [M+H]+=331

### 1-[(3aS,7aS)-1-[5-(difluoromethyl)-6-[2-hydroxy-4-(trifluoromethyl)phenyl]pyridazin-3-yl]-3,3a,4,5,7,7a-hexahydro-2H-pyrrolo[2,3-c]pyridin-6-yl]ethenone and 1-[(3aR,7aR)-1-[5-(difluoromethyl)-6-[2-hydroxy-4-(trifluoromethyl)phenyl]pyridazin-3-yl]-3,3a,4,5,7,7a-hexahydro-2H-pyrrolo[2,3-c]pyridin-6-yl]ethenone (Examples 510 and 511)

Under N₂, to a solution of 1-[rac-(3aR,7aR)-1-[6-chloro-5-(difluoromethyl)pyridazin-3-yl]-3,3a,4,5,7,7a-hexahydro-2H-pyrrolo[2,3-c]pyridin-6-yl]ethanone (190 mg, 0.000574 mol), 2-(4,4,5,5-tetramethyl-1,3,2-dioxaborolan-2-yl)-5-(trifluoromethyl)phenol (0.248 g, 0.000862 mol) in 5 ml of 1,4-dioxane and 1 ml of water were added potassium carbonate (0.238 g, 0.00172 mol), XPhos Pd G2 (0.0226 g, 2.87e-5 mol) and XPhos (0.0137 g, 2.87e-5 mol). The resulting mixture was purged again with N₂, then stirred at 90° C. for 3 h. After cooling down to rt, the resulting mixture was concentrated under reduced pressure. The residue was purified by flash chromatography on silica gel using a gradient DCM/MeOH (100/0 to 90/10) then repurified by reverse phase chromatography (Column YMC—Actus Triart Prep C18-S 150*30 mm 5 μm Flow rate 50 ml/min-Focused gradient MeCN/aq·NH₄HCO₃ 0.2% pH=7.9 Focused gradient from 35/65 to 75/25) to give 53 ml of a solid. This latter was purified by SFC (Stationary phase: Chiralpak® IH 20×250 mm, Mobile phase: CO₂/iPrOH 75/25) to provide 1-[(3aS,7aS)-1-[5-(difluoromethyl)-6-[2-hydroxy-4-(trifluoromethyl)phenyl]pyridazin-3-yl]-3,3a,4,5,7,7a-hexahydro-2H-pyrrolo[2,3-c] pyridin-6-yl]ethenone, first enantiomer eluting Rt=0.65 min (30 mg, Example 510), as a white solid and 1-[(3aR,7aR)-1-[5-(difluoromethyl)-6-[2-hydroxy-4-(trifluoromethyl) phenyl]pyridazin-3-yl]-3,3a,4,5,7,7a-hexahydro-2H-pyrrolo[2,3-c]pyridin-6-yl]ethenone, second enantiomer eluting at Rt=1.03 min (28 mg, Example 511), as a white solid. The absolute configurations cannot be assigned at this stage.

### Example 512 and 513: 1-[(3aS,7aS)-1-[4-(difluoromethyl)-6-(2-hydroxy-4,6-dimethyl-phenyl)pyridazin-3-yl]-3,3a,4,5,7,7a-hexahydro-2H-pyrrolo[2,3-c]pyridin-6-yl]ethenone and 1-[(3aR,7aR)-1-[4-(difluoromethyl)-6-(2-hydroxy-4,6-dimethyl-phenyl)pyridazin-3-yl]-3,3a,4,5,7,7a-hexahydro-2H-pyrrolo[2,3-c]pyridin-6-yl]ethanone

### Int 512.1: 1-[rac-(3aR,7aR)-1-[6-chloro-4-(difluoromethyl)pyridazin-3-yl]-3,3a,4,5,7,7a-hexahydro-2H-pyrrolo[2,3-c]pyridin-6-yl]ethanone

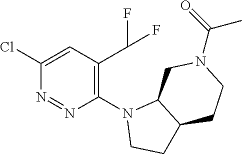 [SMILES: CC(=O)N1CC[C@H]2CCN(c3nnc(Cl)cc3C(F)F)[C@H]2C1]

To a solution of rac-(3aS,7aR)-1-[6-chloro-4-(difluoromethyl)pyridazin-3-yl]-2,3,3a,4,5,6,7,7a-octahydropyrrolo[2,3-c]pyridine;2,2,2-trifluoroacetic acid (Int 487.2-300 mg, 0.000528 mol) and DIPEA (0.273 g, 0.00211 mol) in 4 ml of DCM was added acetyl chloride (0.0539 g, 0.000687 mol) and the resulting mixture was stirred at rt for 2 h. The mixture was diluted with ethyl acetate, washed with a solution of sodium bicarbonate, water and brine, then dried over Na₂SO₄, filtered and concentrated under reduced pressure. The residue was purified by flash chromatography on silica gel using a gradient heptane/ethyl acetate (100/0 to 0/100), then ethyl acetate/methanol (100/0 to 90/10) to give the title compound as an orange solid (109 mg, 50%).

LCMS (Method S): Rt=1.04; MS m/z [M+H]+=331

### 1-[(3aS,7aS)-1-[4-(difluoromethyl)-6-(2-hydroxy-4,6-dimethyl-phenyl)pyridazin-3-yl]-3,3a,4,5,7,7a-hexahydro-2H-pyrrolo[2,3-c]pyridin-6-yl]ethenone and 1-[(3aR,7aR)-1-[4-(difluoromethyl)-6-(2-hydroxy-4,6-dimethyl-phenyl)pyridazin-3-yl]-3,3a,4,5,7,7a-hexahydro-2H-pyrrolo[2,3-c]pyridin-6-yl]ethenone (Examples 512 and 513)

Under N₂, to a solution of 1-[rac-(3aR,7aR)-1-[6-chloro-4-(difluoromethyl)pyridazin-3-yl]-3,3a,4,5,7,7a-hexahydro-2H-pyrrolo[2,3-c]pyridin-6-yl]ethanone (80.0 mg, 0.000189 mol), (2-hydroxy-4,6-dimethyl-phenyl)boronic acid (0.251 g, 0.00151 mol) in 1 ml of 1,4-dioxane and 0.2 ml of water were added potassium carbonate (0.156 g, 0.00113 mol), XPhos Pd G2 (0.0297 g, 3.77e-5 mol) and XPhos (0.0180 g, 3.77e-5 mol). The resulting mixture was purged again with N₂, then stirred at 80° C. for 18 h. After cooling down to rt, the resulting mixture was concentrated under reduced pressure. The residue was purified by reverse phase chromatography (spherical C18 25 μm, 12 g YMC-ODS-25, dry loading (Celite®), mobile phase gradient 0.2% aq. NH₄+HCO₃—/MeCN from 75:25 to 50:50) to give 36 ml of a white solid. This solid was purified by SFC (Stationary phase Chiralpak® IH 20×250 mm, Mobile phase: CO₂/iPrOH 70/30) to provide 1-[(3aS,7aS)-1-[4-(difluoromethyl)-6-(2-hydroxy-4,6-dimethyl-phenyl)pyridazin-3-yl]-3,3a,4,5,7,7a-hexahydro-2H-pyrrolo[2,3-c]pyridin-6-yl]ethenone, first enantiomer eluting Rt=0.67 min (15 mg, Example 512), as a white solid and 1-[(3aR,7aR)-1-[4-(difluoromethyl)-6-(2-hydroxy-4,6-dimethyl-phenyl)pyridazin-3-yl]-3,3a,4,5,7,7a-hexahydro-2H-pyrrolo[2,3-c]pyridin-6-yl]ethenone, second enantiomer eluting at Rt=1.21 min (12 mg, Example 513), as a white solid. The absolute configurations cannot be assigned at this stage.

### Examples 517; 518; 519 and 520: 2-[6-[(4aR,8aS)-7-methyl-2,3,4,4a,5,6,8,8a-octahydro-1,7-naphthyridin-1-yl]pyridazin-3-yl]-3,5-dimethyl-phenol; 2-[6-[(4aS,8aS)-7-methyl-2,3,4,4a,5,6,8,8a-octahydro-1,7-naphthyridin-1-yl]pyridazin-3-yl]-3,5-dimethyl-phenol; 2-[6-[(4aR,8aR)-7-methyl-2,3,4,4a,5,6,8,8a-octahydro-1,7-naphthyridin-1-yl]pyridazin-3-yl]-3,5-dimethyl-phenol and 2-[6-[(4aS,8aR)-7-methyl-2,3,4,4a,5,6,8,8a-octahydro-1,7-naphthyridin-1-yl]pyridazin-3-yl]-3,5-dimethyl-phenol

### Int 517.1: tert-butyl 1-(6-chloropyridazin-3-yl)-2,3,4,4a,5,6,8,8a-octahydro-1,7-naphthyridine-7-carboxylate

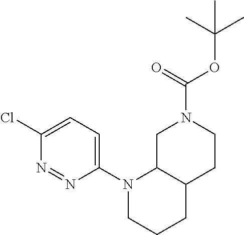 [SMILES: CC(C)(C)OC(=O)N1CCC2CCCN(c3ccc(Cl)nn3)C2C1]

Under Ar, in a vial, to a solution of tert-butyl 2,3,4,4a,5,6,8,8a-octahydro-1H-1,7-naphthyridine-7-carboxylate (807 mg, 3.3562 mmol) in 1 ml of Triethylamine was added 3,6-dichloropyridazine (500 mg, 3.3562 mmol). The vial was sealed and heated at 130° C. for 1 h. The vial was open, and the triethylamine was evaporated under a flux of Ar. Then the heating was continued for 5 h. After cooling down to rt, the mixture was diluted with AcOEt, washed with water and brine, dried over MgSO₄ and concentrated under reduced pressure. The residue was purified by flash chromatography on silica gel eluting with a gradient of DCM/[DCM90/CH₃CN 2.5/MeOH 2.5] (from 100/0 to 0/100) to give the title compound as a yellow oil (320 mg, 27% yield).

LCMS (Method L): Rt=1.10; MS m/z [M+H]⁺=353

### Int 517.2: 1-(6-chloropyridazin-3-yl)-3,4,4a,5,6,7,8,8a-octahydro-2H-1,7-naphthyridine;hydrochloride

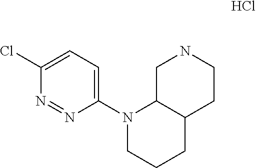 [SMILES: Cl ; Clc1ccc(N2CCCC3CCNCC32)nn1]

To a solution of tert-butyl 1-(6-chloropyridazin-3-yl)-2,3,4,4a,5,6,8,8a-octahydro-1,7-naphthyridine-7-carboxylate (170 mg, 0.4818 mmol, 100 mass %) in 2.4 ml of DCM was added HCl 4N in dioxane (2.4 ml, 9.6356 mmol). The mixture was stirred overnight at rt. The reaction mixture was concentrated under reduced pressure to give the title compound as a cream solid (144 mg, 91% yield).

LCMS (Method L): Rt=0.33; MS m/z [M+H]⁺=253

### Int 517.3: 1-(6-chloropyridazin-3-yl)-7-methyl-2,3,4,4a,5,6,8,8a-octahydro-1,7-naphthyridine

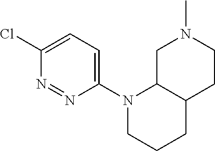 [SMILES: CN1CCC2CCCN(c3ccc(Cl)nn3)C2C1]

To a vigorously stirred mixture of 1-(6-chloropyridazin-3-yl)-3,4,4a,5,6,7,8,8a-octahydro-2H-1,7-naphthyridine;hydrochloride (253 mg, 0.7769 mmol) and Formalin (0.152 ml, 1.9422 mmol, 37 mass %) wad added by fractions sodium triacetoxyborohydride (659 mg, 3.1075 mmol). The reaction mixture was stirred for 2 h at rt. Formalin (40p, 0.080 mmol) and Sodium triacetoxyborohydride (180 mg, 0.850 mmol) were added and the mixture was stirred for 1 h. The reaction mixture was diluted with DCM, washed with an aqueous saturated solution of NaHCO₃, brine, dried over Mg₂SO₄, filtered, and concentrated under reduce pressure to give the title compound as a cream solid (173 mg), which was used without further purification in the next step.

LCMS (Method L): Rt=0.33; MS m/z [M+H]⁺=267

### 2-[6-[(4aR,8aS)-7-methyl-2,3,4,4a,5,6,8,8a-octahydro-1,7-naphthyridin-1-yl]pyridazine-3-yl]-3,5-dimethyl-phenol; 2-[6-[(4aS,8aS)-7-methyl-2,3,4,4a,5,6,8,8a-octahydro-1,7-naphthyridin-1-yl]pyridazin-3-yl]-3,5-dimethyl-phenol; 2-[6-[(4aR,8aR)-7-methyl-2,3,4,4a,5,6,8,8a-octahydro-1,7-naphthyridin-1-yl]pyridazin-3-yl]-3,5-dimethyl-phenol and 2-[6-[(4aS,8aR)-7-methyl-2,3,4,4a,5,6,8,8a-octahydro-1,7-naphthyridin-1-yl]pyridazin-3-yl]-3,5-dimethyl-phenol (Examples 517; 518; 519 and 520)

In a microwave vial, under Ar, to a solution of 1-(6-chloropyridazin-3-yl)-7-methyl-2,3,4,4a,5,6,8,8a-octahydro-1,7-naphthyridine (80 mg, 0.2999 mmol), (2-hydroxy-4,6-dimethyl-phenyl)boronic acid (75 mg, 0.4498 mmol) and sodium carbonate (80 mg, 0.7497 mmol) in 2 ml of 1,4-dioxane and 0.5 ml of water was added tetrakis(triphenylphosphine)palladium(0) (17 mg, 0,015 mmol). The mixture was heated at 110° C. under microwave irradiation for 1 h. Additional Sodium carbonate (40 mg, 0.375 mmol), (2-hydroxy-4,6-dimethyl-phenyl)boronic acid (40 mg, 0.240 mmol) et tetrakis(triphenylphosphine)palladium(0) 10 mg, 0.009 mmol) were added and the mixture was stirred for 1 h at 110° C. under microwave irradiations. After cooling down to rt, the reaction mixture was diluted with AcOEt, washed with water and brine, dried over MgSO₄, concentrated under reduced pressure. The residue was purified by flash chromatography on silica gel eluting with a gradient of DCM/[DCM80/CH₃CN 10/MeOH 10/Et₃n 0.1] (from 80/20 to 0/100) to give, after crystallization, a white solid (50 mg). From this latter, the 4 isomers of compound 3,5-dimethyl-2-[6-(7-methyl-2,3,4,4a,5,6,8,8a-octahydro-1,7-naphthyridin-1-yl)pyridazin-3-yl]phenol were separated by SFC chiral phase LC (Column Chiralpak® IG 5 μm 250×30 mm, Flow rate 115 ml/min, eluent (CO₂ 70%/EtOH+0.1% TEA 30%) and after 23 min (CO₂ 60%/EtOH+0.1% TEA 40%), detection 265 nm) to provide successively first TRANS enantiomer, 2-[6-[(4aR,8aS)-7-methyl-2,3,4,4a,5,6,8,8a-octahydro-1,7-naphthyridin-1-yl]pyridazin-3-yl]-3,5-dimethyl-phenol eluting at rt=14 min (2.8 mg, Example 517) as a white solid, then first CIS enantiomer, 2-[6-[(4aS,8aS)-7-methyl-2,3,4,4a,5,6,8,8a-octahydro-1,7-naphthyridin-1-yl]pyridazin-3-yl]-3,5-dimethyl-phenol, Rt=18.5 min (12.9 mg, Example 518) as a white solid, then second CIS enantiomer, 2-[6-[(4aR,8aR)-7-methyl-2,3,4,4a,5,6,8,8a-octahydro-1,7-naphthyridin-1-yl]pyridazin-3-yl]-3,5-dimethyl-phenol, Rt=22.5 min (12.8 mg, Example 519) as a white solid and second Trans enantiomer, 2-[6-[(4aS,8aR)-7-methyl-2,3,4,4a,5,6,8,8a-octahydro-1,7-naphthyridin-1-yl]pyridazin-3-yl]-3,5-dimethyl-phenol, Rt=32 min (3.1 mg, Example 520) as a white solid. The absolute configurations cannot be assigned at this stage.

Examples 523 and 524 were synthesized analogously to examples 518-519, using a ChiralpaK® AD-H column.

Examples 515 and 516 were synthesized analogously to examples 518-519, using a ChiralpaK®, AY-H column.

### Example 521: 2-[6-[(4aS,8aR)-6-(2-fluoroethyl)-3,4a,5,7,8,8a-hexahydro-2H-pyrido[4,3-b][1,4]oxazin-4-yl]pyridazin-3-yl]-3,5-dimethyl-phenol

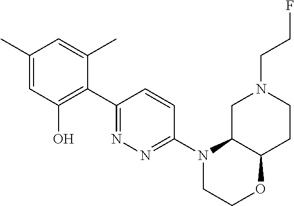 [SMILES: Cc1cc(C)c(-c2ccc(N3CCO[C@@H]4CCN(CCF)C[C@@H]43)nn2)c(O)c1]

### Int 521.1: (4aS,8aR)-4-(6-chloropyridazin-3-yl)-6-(2-fluoroethyl)-3,4a,5,7,8,8a-hexahydro-2H-pyrido[4,3-b][1,4]oxazine

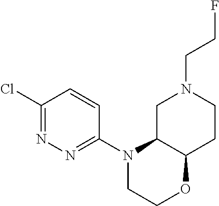 [SMILES: FCCN1CC[C@H]2OCCN(c3ccc(Cl)nn3)[C@H]2C1]

To a solution of (4aS,8aR)-4-(6-chloropyridazin-3-yl)-2,3,4a,5,6,7,8,8a-octahydropyrido[4,3-b][1,4]oxazine (Int 321.2-0.98 mmol, 250 mg) in 10 ml of acetonitrile were added potassium carbonate (1.96 mmol, 274 mg) and 1-fluoro-2-iodo-ethane (0.98 mmol, 80 μl). The mixture was stirred at 60° C. for 3 h then cooled down to rt, diluted with 50 ml of diethylether, 50 ml of EtOAc and 100 ml of water. The organic layer was washed with 100 ml of water, dried over Na₂SO₄, filtered and concentrated under reduced pressure. The residue was purified by flash chromatography on silica gel eluting with a gradient of 0 to 5% of MeOH in DCM to give the title compound (173 mg, 59% yield) as a yellow oil.

LCMS (Method V): Rt=0.59; MS m/z [M+H]⁺=301

### 2-[6-[(4aS,8aR)-6-(2-fluoroethyl)-3,4a,5,7,8,8a-hexahydro-2H-pyrido[4,3-b][1,4]oxazin-4-yl]pyridazin-3-yl]-3,5-dimethyl-phenol (Example 521)

Under Ar, to a solution of (2-hydroxy-4,6-dimethyl-phenyl)boronic acid (1.04 mmol, 172 mg) and (4aS,8aR)-4-(6-chloropyridazin-3-yl)-6-(2-fluoroethyl)-3,4a,5,7,8,8a-hexahydro-2H-pyrido[4,3-b][1,4]oxazine (0.58 mmol, 173 mg) in 8 ml of 1,4-dioxane were added a 2N sodium carbonate aqueous solution (1.55 mmol, 780 μl), then tetrakis(triphenylphosphine)palladium(0) (0.03 mmol, 33 mg). The mixture was stirred at 100° C. for 2 h. After cooling down to rt, the mixture was diluted with 50 ml of EtOAc and 50 ml of diethylether and the organic layer was washed successively with 2×20 ml of a saturated aqueous sodium bicarbonate solution and 20 ml of water. The organic layer was dried over Na₂SO₄, filtered and concentrated under reduced pressure. The residue was purified by flash chromatography on silica gel using a gradient of DCM/MeOH/ACN (from 100/0/0 to 90/5/5) to give the title compound as a white solid (44 mg, 20% yield).

Examples 522, 525 and 527 were synthesized analogously to Example 521.

### Example 528: 5-methyl-2-[4-methyl-6-[rac-(3aS,7aR)-6-isopropyl-3,3a,4,5,7,7a-hexahydro-2H-pyrrolo[2,3-c]pyridin-1-yl]pyridazin-3-yl]phenol

### Int 528.1: rac-(3aS,7aR)-1-(6-chloro-5-methyl-pyridazin-3-yl)-6-isopropyl-3,3a,4,5,7,7a-hexahydro-2H-pyrrolo[2,3-c]pyridine

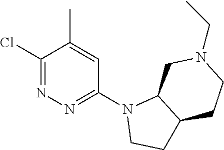 [SMILES: CCN1CC[C@H]2CCN(c3cc(C)c(Cl)nn3)[C@H]2C1]

To a stirred solution of rac-(3aS,7aR)-1-(6-chloro-5-methyl-pyridazin-3-yl)-2,3,3a,4,5,6,7,7a-octahydropyrrolo[2,3-c]pyridine;2,2,2-trifluoroacetic acid (Int 460.2-420 mg, 0.874 mmol) in 8 ml of acetonitrile at rt were added K₂CO₃ (241 mg, 1.75 mmol) and 2-iodopropane (0.087 mL, 0.874 mmol). The reaction mixture was stirred at 50° C. for 5 h. After cooling down to rt, the reaction mixture was poured into water. The aqueous layer was extracted with twice DCM. The combined organic layers were dried over MgSO₄, filtered and concentrated under reduced pressure. The residue was purified by flash chromatography on silica gel using a gradient of DCM/MeOH from 98/2 to 85/15, to give the title compound as a pale-yellow oil (50 mg, 17%).

LCMS (Method T): Rt=5.46; MS m/z [M+H]+=295.1

### 5-methyl-2-[4-methyl-6-[rac-(3aS,7aR)-6-isopropyl-3,3a,4,5,7,7a-hexahydro-2H-pyrrolo [2,3-c]pyridin-1-yl]pyridazin-3-yl]phenol (Example 528)

Under Ar, in a sealed tube, to a solution of rac-(3aS,7aR)-1-(6-chloro-5-methyl-pyridazin-3-yl)-6-isopropyl-3,3a,4,5,7,7a-hexahydro-2H-pyrrolo[2,3-c]pyridine (0.0500 g, 0.000170 mol), K₂CO₃ (0.0313 g, 0.000226 mol) and 5-methyl-2-(4,4,5,5-tetramethyl-1,3,2-dioxaborolan-2-yl)phenol (0.0596 g, 0.000254 mol) in 3.5 ml of 1,4-dioxane and 0.8 ml of water were added XPhos Pd G2 (0.00445 g, 5.65e-6 mol) and XPhos (0.00269 g, 5.65e-6 mol). The mixture was purged again with Ar, then heated at 90° C. for 16 h. After cooling down to rt, the mixture was concentrated under reduced pressure. The residue was purified by flash chromatography on silica gel using a gradient of DCM/MeOH (100/0 to 90/10), then repurified by flash chromatography on silica gel using a gradient of DCM/MeOH (100/0 to 80/20) to give after lyophilization the title compound as a white solid (5 mg, 5%).

### Synthesis of Examples According to Reaction Scheme 2

### Example 1: 1-[6-(2-hydroxy-4,6-dimethyl-phenyl)pyridazin-3-yl]piperidin-4-ol

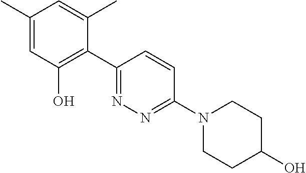 [SMILES: Cc1cc(C)c(-c2ccc(N3CCC(O)CC3)nn2)c(O)c1]

### Int 1.1: 3-chloro-6-(2-methoxy-4,6-dimethyl-phenyl)pyridazine

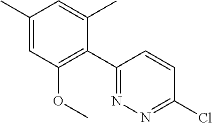 [SMILES: COc1cc(C)cc(C)c1-c1ccc(Cl)nn1]

Under N₂, to a solution of (2-methoxy-4,6-dimethyl-phenyl)boronic acid (5.56 mmol, 1 g) and 3,6-dichloropyridazine (6.67 mmol, 990 mg) in 20 ml of 1,4-dioxane and 5 ml of water was added cesium carbonate (6.67 mmol, 2.2 g). The solution was purged with Ar, degassed by Ar bubbling, then was added [1,1′-bis(diphenylphosphino)ferrocene]dichloropalladium(II) (0.55 mmol, 405 g) and the mixture was stirred at reflux for 1 h. After cooling down to rt, NH₄Cl saturated aqueous solution was added, and the mixture was extracted with EtOAc. The organic layer was dried over Na₂SO₄, filtered and concentrated under reduced pressure. The residue was purified by flash chromatography on silica gel using cyclohexane/EtOAc (from 10/0 to 60/40) to give the title compound (900 mg, 65% yield).

LCMS (Method I): Rt=1.47 min; MS m/z [M+H]+ 249.2

### 1-[6-(2-hydroxy-4,6-dimethyl-phenyl)pyridazin-3-yl]piperidin-4-ol (Example 1)

In a vial, piperidin-4-ol (2.4 mmol, 245 mg) and Et₃N (1.22 mmol, 0.18 ml) were added to a solution of 3-chloro-6-(2-methoxy-4,6-dimethyl-phenyl)pyridazine (0.8 mmol, 200 mg) in 4 ml of n-BuOH. The vial was sealed, and the mixture was heated at 150° C. for 3 h. After cooling down, EtOAc and water are added, and the aqueous layer was extracted with EtOAc. The combined organic layers were dried over Na₂SO₄, filtered and concentrated under reduced pressure. The residue was purified by flash chromatography on silica gel using cyclohexane/EtOAc (from 10/0 to 0/10). 5 ml of DCM are added, and the solution was cooling down to 0° C. Then, a 1M solution of Tribromoboron (2.0 mmol, 2.0 ml) was slowly added and the mixture was stirred at rt for 1.5 h. After cooling down to 0° C., MeOH was added, and the resulting mixture was concentrated under reduced pressure. The residue was purified by prep-HPLC (column: Sunfire prep C18 OBD; 100*30 mm*10 μm; mobile phase: [water (TFA 10⁻² N)-ACN]; B %: 0%-30% in 14 min. at 50 ml/min) to give the title compound as a white solid (76 mg, 32% yield).

Compounds Examples 2 and 3 were synthesized analogously to Example 1.

### Example 26: 2-[6-[2-(1-hydroxy-1-methyl-ethyl)morpholin-4-yl]pyridazin-3-yl]-3-methyl-5-(trifluoromethyl)phenol

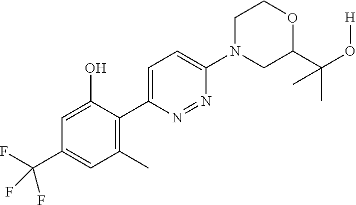 [SMILES: Cc1cc(C(F)(F)F)cc(O)c1-c1ccc(N2CCOC(C(C)(C)O)C2)nn1]

### Int 26.2: 2-(6-chloropyridazin-3-yl)-3-methyl-5-(trifluoromethyl)phenol

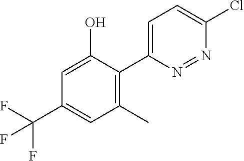 [SMILES: Cc1cc(C(F)(F)F)cc(O)c1-c1ccc(Cl)nn1]

In a microwave vial, under Ar, to a solution of 3-methyl-2-(4,4,5,5-tetramethyl-1,3,2-dioxaborolan-2-yl)-5-(trifluoromethyl)phenol (6.62 mmol, 2 g) and 3,6-dichloropyridazine (9.3 mmol, 1.4 g) in 41 ml of DMF was added a solution of sodium carbonate (14.6 mmol, 1.2 g) in 10 ml of water. The solution was purged with Ar for 10 min, then [1,1′-bis(diphenylphosphino)ferrocene]dichloropalladium(II) (0.23 mmol, 165 mg) was added. The vial was sealed, and the mixture was heated at 110° C. under microwave irradiation for 1 h. After cooling down to rt and concentration under reduced pressure, EtOAc was added, and the resulting mixture was washed with water (3 times) and brine. The organic layer was dried over Na₂SO₄, filtered and concentrated under reduced pressure. The residue was purified by flash chromatography on silica gel using Heptane/EtOAc (8/2). The fractions containing the product were pooled, concentrated under reduced pressure, DCM was added, and the mixture was stirred for 10 min. The suspension was filtered off and dried under reduced pressure to give the title compound as a white solid (598 mg, 31% yield).

LCMS (Method G): Rt=2.51 min; MS m/z [M+H]⁺ 289

### 2-[6-[2-(1-hydroxy-1-methyl-ethyl)morpholin-4-yl]pyridazin-3-yl]-3-methyl-5-(trifluoromethyl)phenol (Example 26)

In a microwave vial, under Ar, 2-(morpholin-2-yl)propan-2-ol hydrochloride (0.38 mmol, 73 mg) and DIPEA (0.76 mmol, 0.14 ml) were added to a solution of 2-(6-chloropyridazin-3-yl)-3-methyl-5-(trifluoromethyl)phenol (0.38 mmol, 110 mg) in 1.1 ml of NMP. The vial was sealed, and the mixture was heated at 180° C. under microwave irradiation for 2 h. After cooling down the solution, EtOAc was added, and the solution was washed with water (3 times) and brine. The organic layer was dried over Na₂SO₄, filtered and concentrated under reduced pressure. The residue was purified by flash chromatography on silica gel using DCM/MeOH/ACN (from 100/0/0 to 90/5/5) to give the title compound as a yellow solid (29 mg, 19% yield).

Compounds Examples 27, 28, 29 and 266 were synthesized analogously to Example 26.

### Examples 52 and 53: separation of enantiomers of 2-[6-[2-(1-hydroxy-1-methyl-ethyl)morpholin-4-yl]pyridazin-3-yl]-3-methyl-5-(trifluoromethyl)phenol

The 2 enantiomers of compound 2-[6-[2-(1-hydroxy-1-methyl-ethyl)morpholin-4-yl]pyridazin-3-yl]-3-methyl-5-(trifluoromethyl)phenol (Example 26, 21 mg) were separated by chiral phase LC (Column Amylose 3, 250*4.6 mm 5 μm, Flow rate 5 ml/min, eluent n-heptane/ethanol (85/15)+0.1% TEA), to give both enantiomers, first eluting Rt=17 min, Example 52 (6.1 mg) and second eluting Rt=21 min, Example 53 (8.3 mg). The absolute configurations cannot be assigned at this stage.

Compounds Examples 59 and 60 were separated enantiomerically from Example 24, analogously to Examples 52 and 53 using a Chiralpak® AD column.

Compounds Examples 94 and 95 were separated enantiomerically from Example 84, analogously to Examples 52 and 53 using a Chiralpak® IC column.

### Example 58: N-[[(3S)-1-[6-(2,4-dichloro-6-hydroxy-phenyl)pyridazin-3-yl]-3-piperidyl]methyl]acetamide

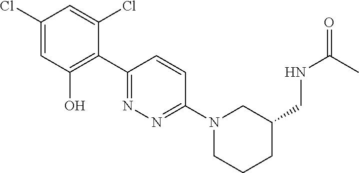 [SMILES: CC(=O)NC[C@@H]1CCCN(c2ccc(-c3c(O)cc(Cl)cc3Cl)nn2)C1]

### Int 58.5: 3,5-dichloro-2-(6-chloropyridazin-3-yl)phenol

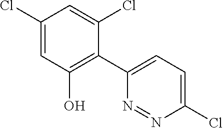 [SMILES: Oc1cc(Cl)cc(Cl)c1-c1ccc(Cl)nn1]

To a solution of (2,4-dichloro-6-hydroxy-phenyl)boronic acid (22.7 mmol) and 3,6-dichloropyridazine (45.4 mmol, 6.76 g) in 140 ml of 1,4-dioxane was added 2N sodium carbonate aqueous solution (61 mmol, 32 ml). The solution was purged with Ar, degassed by Ar bubbling, then was added Tetrakis(triphenylphosphine)palladium(0) (0.907 mmol, 1.05 g) and the mixture was stirred at 75° C. for 3.5 h. The reaction was then cooled down and diluted with 300 ml of EtOAc. The organic layer was successively washed with a 5% aqueous citric acid solution and water, then dried over Na₂SO₄, filtered and concentrated under reduced pressure. The residue was triturated in diethylether, filtered off and the solid washed with diethylether, dried under reduced pressure to give the title compound as a light pink solid (4.27 g, 68% yield)

LCMS (Method I): Rt=1.67 min; MS m/z [M+H]⁺ 275

### N-[[(3S)-1-[6-(2,4-dichloro-6-hydroxy-phenyl)pyridazin-3-yl]-3-piperidyl]methyl]acetamide (Example 58)

A solution of 3,5-dichloro-2-(6-chloropyridazin-3-yl)phenol (0.544 mmol, 150 mg), N-[[(3R)-3-piperidyl]methyl]acetamide;2,2,2-trifluoroacetic acid (1.52 mmol, 586 mg) and Et₃N (2.18 mmol, 0.304 ml) in 3 ml of NMP was stirred at 130° C. for 30 h. The mixture was cooled down, diluted with AcOEt and diethylether, and washed with water (3 times). The organic layer was dried over Na₂SO₄, filtered and concentrated under reduced pressure. The residue was purified by flash chromatography on silica gel eluting with a gradient of 0 to 30% of EtOAc in cyclohexane to give the title compound as a yellow solid (82 mg, 38% yield).

Compounds Examples 57, 64 and 66 were synthesized analogously to Example 58.

### Example 18: 2-[6-[(3S)-3-(aminomethyl)-1-piperidyl]pyridazin-3-yl]-3,5-dichloro-phenol

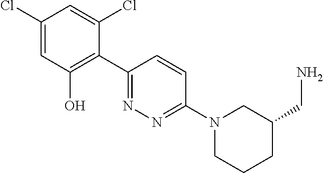 [SMILES: NC[C@@H]1CCCN(c2ccc(-c3c(O)cc(Cl)cc3Cl)nn2)C1]

### Int 18.1: tert-butyl N-[[(3S)-1-[6-(2,4-dichloro-6-hydroxy-phenyl)pyridazin-3-yl]-3-piperidyl]methyl]carbamate

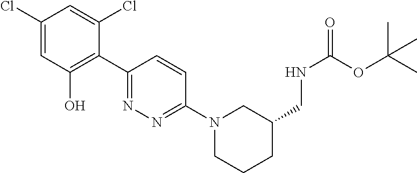 [SMILES: CC(C)(C)OC(=O)NC[C@@H]1CCCN(c2ccc(-c3c(O)cc(Cl)cc3Cl)nn2)C1]

A solution of 3,5-dichloro-2-(6-chloropyridazin-3-yl)phenol (0.544 mmol, 150 mg), tert-butyl N-[[(3R)-3-piperidyl]methyl]carbamate (1.14 mmol, 245 mg) in 2.5 ml of NMP was stirred at 130° C. for 2 h. The mixture was cooled down, diluted with AcOEt and diethylether, then washed with water (3 times). The organic layer was dried over Na₂SO₄, filtered and concentrated under reduced pressure. The residue was purified by flash chromatography on silica gel eluting with a gradient of 0 to 20% of EtOAc in cyclohexane to give the title compound as a yellow solid (222 mg, 90% yield).

LCMS (Method O): Rt=1.47 min; MS m/z [M+H]⁺ 453

### 2-[6-[(3S)-3-(aminomethyl)-1-piperidyl]pyridazin-3-yl]-3,5-dichloro-phenol (Example 18)

A solution of tert-butyl N-[[(3S)-1-[6-(2,4-dichloro-6-hydroxy-phenyl)pyridazin-3-yl]-3-piperidyl]methyl]carbamate (0.49 mmol, 222 mg) in 18 ml of DCM and 2 ml of TFA was stirred at rt for 3 h. The solution was concentrated under reduced pressure. The residue was purified by prep-HPLC (column: Sunfire prep C18 OBD; 250*50 mm*10 μm; mobile phase: [water (TFA 10⁻² N)-ACN]; B %: 10%-100%, 25 min. (50 ml/min). The pure fractions were extracted in DCM/NaHCO₃ sat and the organic layer was dried over Na₂SO₄, filtered and concentrated under reduced pressure to give the title compound as a white solid (106 mg, 61% yield).

Compounds Examples 17, 19, 21, 22, 23, 25, 32 to 43, 46, 47, 49, 50, 51, and 55 were synthesized analogously to Example 18.

### Example 89: 2-[6-[(3R)-3-(aminomethyl)-1-piperidyl]pyridazin-3-yl]-3,5-dimethyl-phenol; 2,2,2-trifluoroacetic acid

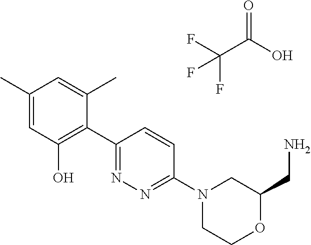 [SMILES: Cc1cc(C)c(-c2ccc(N3CCO[C@H](CN)C3)nn2)c(O)c1 ; O=C(O)C(F)(F)F]

### Int 89.1: 2-(6-chloropyridazin-3-yl)-3,5-dimethyl-phenol

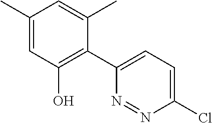 [SMILES: Cc1cc(C)c(-c2ccc(Cl)nn2)c(O)c1]

In a microwave vial, to a solution of (2-hydroxy-4,6-dimethyl-phenyl)boronic acid (4.15 mmol, 690 mg) and 3,6-dichloropyridazine (3.20 mmol, 500 mg) in 20 ml of 1,4-dioxane and 5 ml of water was added sodium carbonate (9.6 mmol, 790 mg). The solution was purged with Ar, degassed by Ar bubbling, then was added tetrakis(triphenylphosphine)palladium(0) (0.16 mmol, 180 mg). The vial was sealed, and the mixture was heated at 100° C. under microwave irradiation for 1 h. After cooling down, the solution was concentrated under reduced pressure, then 25 ml of DCM was added. The organic layer was washed with water (3 times), dried over Na₂SO₄, filtered and concentrated under reduced pressure. The residue was triturated in pentane, filtered off and the solid washed with pentane, dried under reduced pressure to give the title compound as a light brown solid (440 mg, 58% yield).

LCMS (Method G): Rt=1.54 min; MS m/z [M+H]⁺ 235

### Int 89.2: tert-butyl N-[[(2R)-4-[6-(2-hydroxy-4,6-dimethyl-phenyl)pyridazin-3-yl]morpholin-2-yl]methyl]carbamate

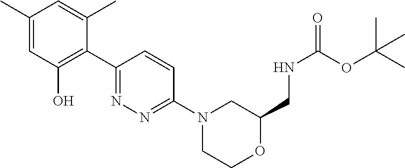 [SMILES: Cc1cc(C)c(-c2ccc(N3CCO[C@H](CNC(=O)OC(C)(C)C)C3)nn2)c(O)c1]

In a microwave vial, under Ar, (S)-tert-butyl(morpholin-2-ylmethyl)carbamate (0.56 mmol, 126 mg) and Et₃N (1.22 mmol, 0.18 ml) were added to a solution of 2-(6-chloropyridazin-3-yl)-3,5-dimethyl-phenol (0.56 mmol, 130 mg) in 5 ml of n-BuOH. The vial was sealed, and the mixture was heated at 180° C. under microwave irradiation for 4 h. After cooling down, the solution was concentrated under vacuum. 15 ml of EtOAc was added and the solution was washed with water (3 times) and brine. The organic layer was dried over Na₂SO₄, filtered and concentrated under reduced pressure.

The residue was purified by flash chromatography on silica gel using DCM/MeOH/ACN (from 100/0/0 to 94/3/3) to give the title compound as a yellow solid (48 mg, 21% yield).

LCMS (Method G): Rt=1.38 min; MS m/z [M+H]⁺ 415

### 2-[6-[(3R)-3-(aminomethyl)-1-piperidyl]pyridazin-3-yl]-3,5-dimethyl-phenol;2,2,2-trifluoroacetic acid (Example 89)

A solution of tert-butyl N-[[(3R)-1-[6-(2-hydroxy-4,6-dimethyl-phenyl)pyridazin-3-yl]-3-piperidyl]methyl]carbamate (0.12 mmol, 49 mg) in 1 ml of DCM and 0.1 ml of TFA was stirred at rt for 16 h. The solution was concentrated under reduced pressure. 1 ml of MeOH was added and the solution was concentrated under reduced pressure (3 times). The residue was triturated in 2 ml of pentane, filtered off and dried under reduced pressure at 40° C. for 4 h to give the titled compound a light pink solid (27 mg, 53% yield).

Compound Example 107 was synthesized analogously to Example 89.

### Example 87: 2-[6-[(2S)-2-(aminomethyl)morpholin-4-yl]pyridazin-3-yl]-3,5-dimethyl-phenol; 2,2,2-trifluoroacetic acid

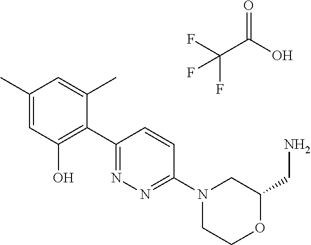 [SMILES: Cc1cc(C)c(-c2ccc(N3CCO[C@@H](CN)C3)nn2)c(O)c1 ; O=C(O)C(F)(F)F]

### Int 87.1: 2-(6-chloropyridazin-3-yl)-3,5-dimethyl-phenol

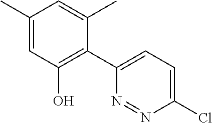 [SMILES: Cc1cc(C)c(-c2ccc(Cl)nn2)c(O)c1]

In a microwave vial, to a solution of (2-hydroxy-4,6-dimethyl-phenyl)boronic acid (4.15 mmol, 690 mg) and 3,6-dichloropyridazine (3.20 mmol, 500 mg) in 20 ml of 1,4-dioxane and 5 ml of water was added sodium carbonate (9.6 mmol, 790 mg). The solution was purged with Ar, degassed by Ar bubbling, then was added Tetrakis(triphenylphosphine)palladium(0) (0.16 mmol, 180 mg). The vial was sealed, and the mixture was heated at 100° C. under microwave irradiation for 1 h. After cooling down, the solution was concentrated under reduced pressure, then 25 ml of DCM was added. The organic layer was washed with water (3 times), dried over Na₂SO₄, filtered and concentrated under reduced pressure. The residue was triturated in pentane, filtered off and the solid washed with pentane, dried under reduced pressure to give the title compound as a light brown solid (440 mg, 58% yield).

LCMS (Method G): Rt=1.54 min; MS m/z [M+H]⁺ 235

### Int 87.2: tert-butyl N-[[(2S)-4-[6-(2-hydroxy-4,6-dimethyl-phenyl)pyridazin-3-yl]morpholin-2-yl]methyl]carbamate

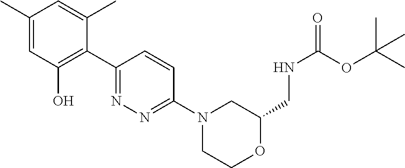 [SMILES: Cc1cc(C)c(-c2ccc(N3CCO[C@@H](CNC(=O)OC(C)(C)C)C3)nn2)c(O)c1]

In a microwave vial, under Ar, (R)-tert-butyl (morpholin-2-ylmethyl) carbamate (0.51 mmol, 116 mg) and Et₃N (1.12 mmol, 0.16 ml) were added to a solution of 2-(6-chloropyridazin-3-yl)-3,5-dimethyl-phenol (0.51 mmol, 116 mg) in 5 ml of n-BuOH. The vial was sealed, and the mixture was heated at 180° C. under microwave irradiation for 3 h. After cooling down, the solution was concentrated under vacuum. 15 ml of EtOAc was added and the solution was washed with water (3 times) and brine. The organic layer was dried over Na₂SO₄, filtered and concentrated under reduced pressure. The residue was purified by flash chromatography on silica gel using DCM/MeOH/ACN (from 100/0/0 to 96/2/2) to give the title compound as a yellow solid (63 mg, 29% yield).

LCMS (Method G): Rt=1.36 min; MS m/z [M+H]⁺=415

### 2-[6-[(2S)-2-(aminomethyl)morpholin-4-yl]pyridazin-3-yl]-3,5-dimethyl-phenol;2,2,2-trifluoroacetic acid (Example 87)

To a solution of tert-butyl N-[[(2S)-4-[6-(2-hydroxy-4,6-dimethyl-phenyl)pyridazin-3-yl]morpholin-2-yl]methyl]carbamate (0.152 mmol, 63 mg) in 1 ml of dichloromethane, 0.12 ml of TFA was added dropwise. The resulting solution was stirred at room temperature for 16 h. After completion of reaction, the solution was concentrated under reduced pressure, 1 ml of MeOH was added and the solution was concentrated under reduced pressure (3 times). The residue was suspended in 2 ml of diethyl ether and stirred overnight at room temperature. The suspension was filtered off and dried under reduced pressure to give the titled compound as a light pink solid (34 mg, 52% yield).

### Example 190: 2-[6-[rac-(4aR,7aR)-3,4a,5,6,7,7a-hexahydro-2H-pyrrolo[3,4-b][1,4]oxazin-4-yl]pyridazin-3-yl]-3,5-dimethylphenol

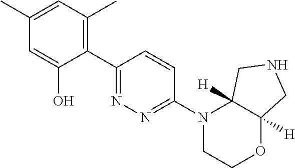 [SMILES: Cc1cc(C)c(-c2ccc(N3CCO[C@@H]4CNC[C@H]43)nn2)c(O)c1]

### Int 190.1: 2-(6-chloropyridazin-3-yl)-3,5-dimethyl-phenol

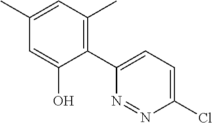 [SMILES: Cc1cc(C)c(-c2ccc(Cl)nn2)c(O)c1]

In a microwave vial under Ar, to a mixture of (2-hydroxy-4,6-dimethyl-phenyl)boronic acid (429 mg, 2.58 mmol) in 8 ml of 1,4-dioxane was added 3-bromo-6-chloro-pyridazine (500 mg, 2.58 mmol) and K₃PO₄ (1.65 g, 7.75 mmol) in 2 ml of water. The mixture was purged with N₂. Then Pd¹⁸ (185 mg, 0.284 mmol) was added and purged again with N₂. The mixture was then heated at 90° C. for 18 h. After cooling to rt, the reaction mixture was filtered and concentrated under reduced pressure. The residue was purified by flash chromatography on silica gel using heptane/EtOAc (100/0 to 30/70) to give the title compound as an orange solid (120 mg, 19% yield) LCMS (Method S): Rt=1.12 min; MS m/z [M+H]⁺=235.1

### Int 190.2: tert-butyl rac-(4aR,7aS)-1-[6-(2-hydroxy-4,6-dimethyl-phenyl)pyridazin-3-yl]-3,4,4a,5,7,7a-hexahydro-2H-pyrrolo[3,4-b]pyridine-6-carboxylate

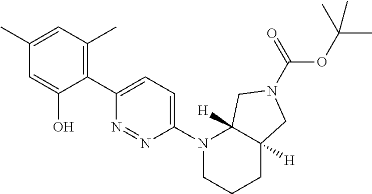 [SMILES: Cc1cc(C)c(-c2ccc(N3CCC[C@@H]4CN(C(=O)OC(C)(C)C)C[C@H]43)nn2)c(O)c1]

Under Ar, to a stirred solution of 2-(6-chloropyridazin-3-yl)-3,5-dimethyl-phenol (154 mg, 0.657 mmol) in 5 ml of tetrahydrofuran at rt were added tert-butyl (4aR,7aR)-3,4,4a,5,7,7a-hexahydro-2H-pyrrolo[3,4-b][1,4]oxazine-6-carboxylate (150 mg, 0.657 mmol), LiHMDS (1M in THF) (3.55 mL, 0.3.55 mmol) and BrettPhos Pd G3 (0.0298 g, 0.0329 mmol). The reaction mixture was stirred at 100° C. for 18 h. After cooling to rt, the reaction mixture was concentrated under reduced pressure. The residue was purified by flash chromatography on silica gel using DCM/MeOH (100/0 to 95/5) to give the title compound as an orange solid (30 mg, 10% yield)

LCMS (Method S): Rt=1.34 min; MS m/z [M+H]⁺=427.4

### 2-[6-[rac-(4aR,7aR)-3,4a,5,6,7,7a-hexahydro-2H-pyrrolo[3,4-b][1,4]oxazin-4-yl]pyridazin-3-yl]-3,5-dimethylphenol (Example 190)

At 0° C., to a stirred solution of tert-butyl rac-(4aR,7aR)-4-[6-(2-hydroxy-4,6-dimethyl-phenyl)pyridazin-3-yl]-2,3,4a,5,7,7a-hexahydropyrrolo[3,4-b][1,4]oxazine-6-carboxylate (30.0 mg, 0.007 mmol) in 1 ml of DCM was added TFA (0.0052 mL, 0.703 mmol). The reaction mixture was stirred at 30° C. for 18 h. After cooling to rt, the mixture was concentrated under reduced pressure. The residue was then taken up in water and basified with an aqueous solution of K₂CO₃ 10%. The mixture was extracted with DCM/MeOH (80/20), dried over MgSO₄, filtered, and concentrated under reduced pressure. The residue was purified by reverse phase YMC—Actus Triart Prep C18-S 150*30 mm 5 μm Flow rate 50 ml/min-Focused gradient MeCN/aq·NH₄HCO₃ 0.2% pH=7.9 Focused gradient from 15/85 to 45/55, to give after lyophilisation the title compound as a white powder (3 mg, 11% yield).

### Example 122: 1-[6-(2-hydroxy-4,6-dimethyl-phenyl)pyridazin-3-yl]-3-(hydroxymethyl) pyrrolidin-2-one

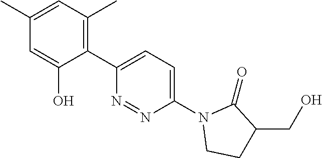 [SMILES: Cc1cc(C)c(-c2ccc(N3CCC(CO)C3=O)nn2)c(O)c1]

In a microwave vial, under Ar, a mixture of 2-(6-chloropyridazin-3-yl)-3,5-dimethyl-phenol (0.89 mmol, 210 mg), 3-(hydroxymethyl)pyrrolidin-2-one (1.34 mmol, 155 mg), potassium phosphate tribasic (1.79 mmol, 380 mg), 1,1′bis(diphenylphosphino)ferrocene (0.16 mmol, 89 mg) and palladium acetate (0.05 mmol, 12 mg) in 3 ml of 1,4-dioxane was heated at 110° C. under microwave irradiation for 2 h. After cooling to rt, the reaction mixture was diluted with EtOAc and filtered. The solid was washed with EtOAc, and the filtrate was concentrated under reduced pressure. The residue was purified by flash chromatography on silica gel using DCM/MeOH/ACN (from 100/0/0 to 94/3/3) to give the title compound as a brown solid (83 mg, 30% yield).

Compounds Examples 131, 132, 235, 250, 264 and 269 were synthesized analogously to Example 122.

### Examples 137 and 138: (4R)-1-[6-(2-hydroxy-4,6-dimethyl-phenyl)pyridazin-3-yl]-4-(hydroxymethyl)pyrrolidin-2-one (Compound 137) and (4S)-1-[6-(2-hydroxy-4,6-dimethyl-phenyl)pyridazin-3-yl]-4-(hydroxymethyl)pyrrolidin-2-one (compound 138) were separated from racemic compound 1-[6-(2-hydroxy-4,6-dimethyl-phenyl)pyridazin-3-yl]-4-(hydroxymethyl)pyrrolidin-2-one which was synthesized analogously to ex 122 using 4-(hydroxymethyl)pyrrolidin-2-one

The 2 enantiomers of compound 1-[6-(2-hydroxy-4,6-dimethyl-phenyl)pyridazin-3-yl]-4-(hydroxymethyl)pyrrolidin-2-one (70 mg) were separated by preparative chiral phase LC (Column Chiralcel® OD 5 μm, 350×30 mm, Flow rate 45 ml/min, eluent (n-heptane/ethanol 20/80)+0.10% TEA, to give both enantiomers, first eluting Rt=6.0 min, Example 137 (15 mg) and second eluting Rt=13 min, Example 138 (15.5 mg). The absolute configurations cannot be assigned at this stage.

### Examples 139 and 140: separation of enantiomers of 1-[6-(2-hydroxy-4,6-dimethyl-phenyl)pyridazin-3-yl]-3-(hydroxymethyl)pyrrolidin-2-one, namely (3R)-1-[6-(2-hydroxy-4,6-dimethyl-phenyl)pyridazin-3-yl]-3-(hydroxymethyl)pyrrolidin-2-one (Example 139) and (3S)-1-[6-(2-hydroxy-4,6-dimethyl-phenyl)pyridazin-3-yl]-3-(hydroxymethyl) pyrrolidin-2-one (Example 140)

The 2 enantiomers of compound 1-[6-(2-hydroxy-4,6-dimethyl-phenyl)pyridazin-3-yl]-3-(hydroxymethyl)pyrrolidin-2-one (Example 122, 75 mg) were separated by preparative chiral phase LC (Column Chiralcel® OJ-H 5 μm, 350×30 mm, Flow rate 45 ml/min, eluent (n-heptane/ethanol 60/40)+0.10% TEA, to give both enantiomers, first eluting Rt=8.6 min, Example 139 (20 mg) and second eluting Rt=12.7 min, Example 140 (28.5 mg). The absolute configurations cannot be assigned at this stage.

### Example 161: 3,5-dichloro-2-[6-[2-(fluoromethyl)morpholin-4-yl]pyridazin-3-yl]phenol

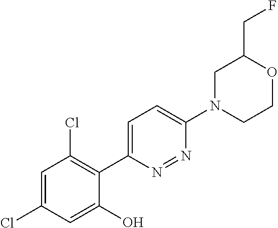 [SMILES: Oc1cc(Cl)cc(Cl)c1-c1ccc(N2CCOC(CF)C2)nn1]

In a microwave vial, under Ar, a suspension of 3,5-dichloro-2-(6-chloropyridazin-3-yl)phenol (0.18 mmol, 50 mg), 2-(fluoromethyl)morpholine hydrochloride (0.27 mmol, 42 mg) and diisopropylethylamine (0.40 mmol, 0.07 ml) in 0.5 ml of n-butanol was heated at 160° C. under microwave irradiation for 1 h. In another microwave vial, under Ar, a suspension of 3,5-dichloro-2-(6-chloropyridazin-3-yl)phenol (0.18 mmol, 50 mg), 2-(fluoromethyl)morpholine hydrochloride (0.27 mmol, 42 mg) and diisopropylethylamine (0.40 mmol, 0.07 ml) in 0.5 ml of acetonitrile was heated at 160° C. under microwave irradiation for 1 h. The reaction mixtures were combined, diluted with CH₂Cl₂ and the organic layer was washed with 45 ml of water, filtered on hydrophobic column and the filtrate was concentrated under reduced pressure. The residue was purified by flash chromatography on silica gel using DCM/MeOH/ACN (from 100/0/0 to 96/2/2) to give the title compound as a white solid (84 mg, 65% yield).

### Examples 237 and 238: separation of enantiomers of 3,5-dichloro-2-[6-[2-(fluoromethyl)morpholin-4-yl]pyridazin-3-yl]phenol, namely 3,5-dichloro-2-[6-[(2R)-2-(fluoromethyl)morpholin-4-yl]pyridazin-3-yl]phenol (example 237) and 3,5-dichloro-2-[6-[(2S)-2-(fluoromethyl)morpholin-4-yl]pyridazin-3-yl]phenol (example 238)

The 2 enantiomers of compound 3,5-dichloro-2-[6-[2-(fluoromethyl)morpholin-4-yl]pyridazin-3-yl]phenol (example 161, 60 mg) were separated by preparative chiral phase LC (Column Chiralpak® AY-H 5 μm, 250×30 mm, Flow rate 40 ml/min, eluent (n-heptane/ethanol 90/10)+0.1% TEA to give both enantiomers, first eluting Rt=21.5 min, Example 237 (22.1 mg) and second eluting Rt=24.8 min, Example 238 (25.4 mg). The absolute configurations cannot be assigned at this stage.

Compounds examples 219 and 220 were separated enantiomerically from example 174, analogously to examples 237 and 238 using a Chiralpak® IB—N column.

### Example 239: 1-[6-(2,4-dichloro-6-hydroxy-phenyl)pyridazin-3-yl]hexahydropyrimidin-2-one

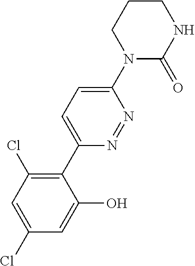 [SMILES: O=C1NCCCN1c1ccc(-c2c(O)cc(Cl)cc2Cl)nn1]

In a microwave vial, under Ar, a suspension of tetrahydro-2(1H)-pyrimidinone (3.63 mmol, 363 mg), 3,5-dichloro-2-(6-chloropyridazin-3-yl)phenol (1.81 mmol, 500 mg), cesium carbonate (3.63 mmol, 1.18 g), Pd₂(dba)₃ (0.18 mmol, 166 mg) and 4,5-bis(diphenylphosphino)-9,9-dimethylxanthene (0.36 mmol, 210 mg) in 10 ml of 1,4-dioxane was heated at 110° C. under microwave irradiation for 2 h. After cooling to rt, the reaction mixture was diluted with EtOAc, filtered and the filtrate was concentrated under reduced pressure. The residue was purified by flash chromatography on silica gel using DCM/EtOAc (from 100/0 to 0/100). The solid obtained after filtration was also purified by flash chromatography on silica gel using DCM/EtOAc (from 100/0 to 0/100). Fractions from both purifications containing expected compound were combined and concentrated under reduced pressure. The residue obtained was purified by flash chromatography on silica gel using DCM/EtOAc (from 100/0 to 0/100) to give the title compound as a brown solid (46 mg, 7% yield).

### Example 114: 2-[6-[1-(hydroxymethyl)-6-azabicyclo[3.2.1]octan-6-yl]pyridazin-3-yl]-3,5-dimethyl-phenol

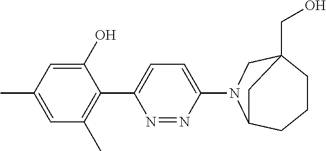 [SMILES: Cc1cc(C)c(-c2ccc(N3CC4(CO)CCCC3C4)nn2)c(O)c1]

A solution of 2-(6-chloropyridazin-3-yl)-3,5-dimethyl-phenol (100 mg, 0.4261 mmol), 6-azabicyclo[3.2.1]octan-1-ylmethanol;hydrochloride (98 mg, 0.5539 mmol) and Triethylamine (198 μl, 1.4062 mmol) in 2.5 ml of 1-Butanol was heated at 150° C. by microwave irradiation for 2 h. 6-azabicyclo[3.2.1]octan-1-ylmethanol;hydrochloride (30 mg) was added and the mixture was heated for 1 h at 150° C. After cooling to rt, the reaction mixture was diluted with water and extracted with AcOEt. The organic layer was washed with brine, dry over Mg₂SO₄, filtered, and concentrated under reduce pressure. The residue was purified by flash chromatography on silica gel using DCM/MeOH (100/0 to 95/05) to give after crystallization in diethyl ether, the title compound as a beige solid (20 mg, 13% yield).

Compounds Examples 115, 117, 120, 174, 206 and 207 were synthesized analogously to Example 114.

### Example 164: N-[[(3S)-1-[6-[2-hydroxy-6-methyl-4-(trifluoromethyl)phenyl]pyridazin-3-yl]-3-piperidyl]methyl]acetamide

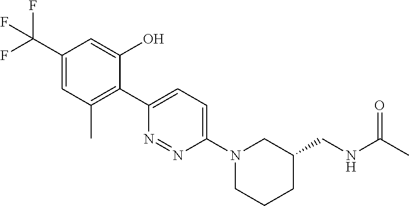 [SMILES: CC(=O)NC[C@@H]1CCCN(c2ccc(-c3c(C)cc(C(F)(F)F)cc3O)nn2)C1]

### Int 164.1: tert-butyl (3S)-3-(acetamidomethyl)piperidine-1-carboxylate

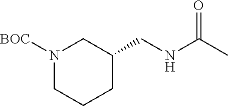 [SMILES: CC(=O)NC[C@@H]1CCCN(C(=O)OC(C)(C)C)C1]

To a solution of tert-butyl (3S)-3-(aminomethyl)piperidine-1-carboxylate (1 g, 4.6663 mmol) was solubilized in 20 ml of DCM id added triethylamine (656 μl, 4.6663 mmol) and N,N-dimethylpyridin-4-amine (29 mg, 0.2333 mmol). Acetic anhydride (916 μl, 6.9994 mmol) was slowly added to the solution and the mixture was stirred overnight. The reaction mixture was diluted with DCM, washed with a saturated aqueous solution of NaHCO₃ and brine, dried on MgSO₄ and concentrated under reduced pressure. The residue was purified by flash chromatography on silica gel using DCM/MeOH (from 100/0 to 95/5) to give the title compound as a colorless oil (1.10 g, 91% yield).

1H NMR (400 MHz, CDCl₃, 30° C.) δ ppm 1.15-1.27 (m, 1H), 1.29-1.38 (m, 1H), 1.39 (s, 9H), 1.50-1.78 (m, 3H), 1.92 (s, 3H), 2.74-3.34 (m, 4H), 3.44-3.70 (m, 2H), 5.50-6.04 (m, 1H)

### Int 164.2: N-[[(3R)-3-piperidyl]methyl]acetamide;2,2,2-trifluoroacetic acid

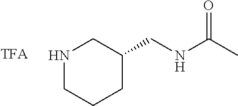

tert-butyl (3S)-3-(acetamidomethyl)piperidine-1-carboxylate (300 mg, 1.1703 mmol) was solubilized in 15 ml of DCM. The mixture was cooled to 0° C. and 1.5 ml of TFA was slowly added. After 3 h stirring at room temperature, the reaction mixture was concentrated under reduce pressure to give the title compound as a colourless oil (460 mg, yield >th), which was used without further purification in the next step.

### N-[[(3S)-1-[6-[2-hydroxy-6-methyl-4-(trifluoromethyl)phenyl]pyridazin-3-yl]-3-piperidyl]methyl]acetamide (Example 164)

In a microwave vial, under Ar, to a solution of 2-(6-chloropyridazin-3-yl)-3-methyl-5-(trifluoromethyl)phenol (130 mg, 0.4504 mmol) in 2.5 ml of 1-Butanol were added N-[[(3R)-3-piperidyl]methyl]acetamide;2,2,2-trifluoroacetic acid (304 mg, 1.1259 mmol) and triethylamine (314 μl, 2.2518 mmol). The mixture was heated at 160° C. under microwave irradiation for 2 h. After cooling to rt, the reaction mixture was diluted with water and extracted with AcOEt. The organic layer was washed with brine, dried over MgSO₄, filtered and concentrated under reduce pressure. The residue was purified by flash chromatography on silica gel using DCM/MeOH (100/0 to 95/5) to give the title compound as a white solid (101 mg, 55% yield).

Compound Example 167 was synthesized analogously to Example 164.

### Example 217: 1-[6-(2-hydroxy-4,6-dimethyl-phenyl)pyridazin-3-yl]-3,4-dihydro-2H-quinolin-7-ol

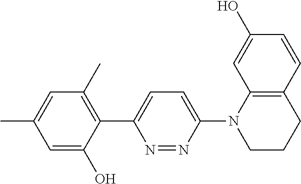 [SMILES: Cc1cc(C)c(-c2ccc(N3CCCc4ccc(O)cc43)nn2)c(O)c1]

In a microwave vial under Ar, 2-(6-chloropyridazin-3-yl)-3,5-dimethyl-phenol (130 mg, 0.5539 mmol) and 1,2,3,4-tetrahydroquinolin-7-ol (207 mg, 1.3849 mmol) in 2.5 ml of 1-Butanol were heated at 160° C. under microwave irradiation for 2 h. After cooling to rt, the reaction mixture was diluted with AcOEt, the organic layer was washed with water and brine, dried over MgSO₄ and concentrated under reduced pressure. The residue was purified by flash chromatography on silica gel using DCM/MeOH (100/0 to 97/3) to give the title compound as a white solid (23 mg, 12% yield).

Compounds Examples 230, 231, 232, 236, 240, 241, 242, 253, 254 and 272 were synthesized analogously to Example 217.

### Examples 233 and 234: 2-[6-[(2R)-2-(2-hydroxyethyl)morpholin-4-yl]pyridazin-3-yl]-3,5-dimethyl-phenol and 2-[6-[(2S)-2-(2-hydroxyethyl)morpholin-4-yl]pyridazin-3-yl]-3,5-dimethyl-phenol

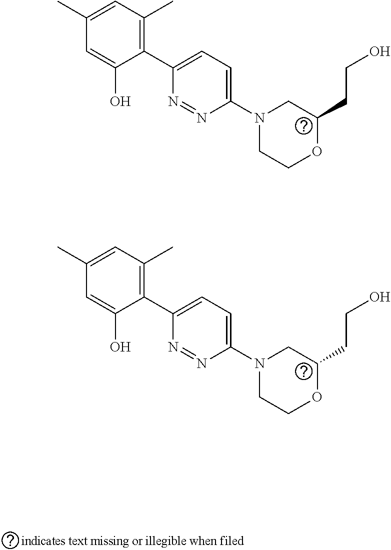 [SMILES: Cc1cc(C)c(-c2ccc(N3CCO[C@H](CCO)C3)nn2)c(O)c1 ; Cc1cc(C)c(-c2ccc(N3CCO[C@@H](CCO)C3)nn2)c(O)c1]

In a microwave vial, under Ar, to a solution of 2-(6-chloropyridazin-3-yl)-3,5-dimethyl-phenol (300 mg, 1.2783 mmol) in 6 ml of 1-Butanol were added 2-morpholin-2-ylethanol (419 mg, 3.1958 mmol) and Et₃N (445 μl, 3.1958 mmol). The mixture was heated at 160° C. under microwave irradiation for 2 h. After cooling to rt, the reaction mixture was diluted with AcOEt, the organic layer was washed with water and brine, dried over and concentrated under reduced pressure. The residue was purified by flash chromatography on silica gel using DCM/MeOH (100/0 to 95/5) to give 2-[6-[2-(2-hydroxyethyl)morpholin-4-yl]pyridazin-3-yl]-3,5-dimethyl-phenol (180 mg, 43% yield).

The 2 enantiomers of 2-[6-[2-(2-hydroxyethyl)morpholin-4-yl]pyridazin-3-yl]-3,5-dimethyl-phenol were separated by chiral phase LC (Column Cellulose 4, 350×76.5 mm, Flow rate 400 ml/min, eluent n-heptane/ethanol (75/25)+0.1% TEA), to give both enantiomers, first eluting Rt=18.5 min, Example 233 (81 mg) and second eluting Rt=25.7 min, Example 234 (83 mg). Absolute configuration was unknown for the two compounds.

### Examples 277 and 278: N-[2-[(2R)-4-[6-[2-hydroxy-6-methyl-4-(trifluoromethyl)phenyl]pyridazin-3-yl]morpholin-2-yl]ethyl]acetamide and N-[2-[(2S)-4-[6-[2-hydroxy-6-methyl-4-(trifluoromethyl)phenyl]pyridazin-3-yl]morpholin-2-yl]ethyl]acetamide

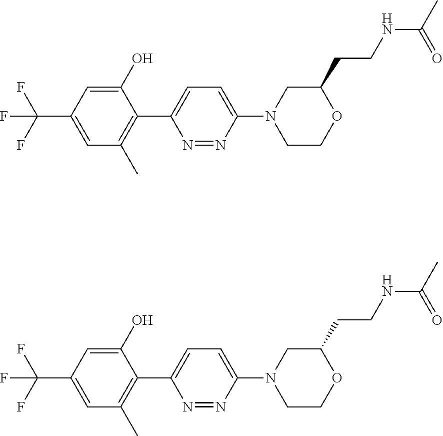 [SMILES: CC(=O)NCC[C@@H]1CN(c2ccc(-c3c(C)cc(C(F)(F)F)cc3O)nn2)CCO1 ; CC(=O)NCC[C@H]1CN(c2ccc(-c3c(C)cc(C(F)(F)F)cc3O)nn2)CCO1]

### Int 277.1: tert-butyl 2-(2-acetamidoethyl)morpholine-4-carboxylate

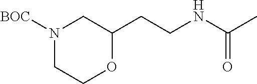 [SMILES: CC(=O)NCCC1CN(C(=O)OC(C)(C)C)CCO1]

To a solution of tert-butyl 2-(2-aminoethyl)morpholine-4-carboxylate (1 g, 4.3421 mmol) in 20 ml of DCM was added Et₃N (610 μl, 4.3421 mmol) and N,N-dimethylpyridin-4-amine (27 mg, 0.2171 mmol). Acetic anhydride (664 mg, 6.5131 mmol) was slowly added, and the mixture was stirred overnight at rt. The reaction mixture was diluted with DCM, the organic layer was washed with water and brine, dried over MgSO₄ and concentrated under reduced pressure. The residue was purified by flash chromatography on silica gel using DCM/MeOH (100/0 to 95/5) to give the title compound as colorless oil (1.09 g, 92% yield). 1H NMR (400 MHz, CDCl₃, 30° C.) δ ppm 1.39 (s, 9H), 1.46-1.57 (m, 1H), 1.58-1.71 (m, 1H), 1.89 (s, 3H), 2.55 (br t, J=11.3 Hz, 1H), 2.84 (br t, J=11.3 Hz, 1H), 3.14-3.24 (m, 1H), 3.31-3.54 (m, 3H), 3.77-3.85 (m, 3H), 5.89 (br s, 1H)

### Int 277.2: N-(2-morpholin-2-ylethyl)acetamide;2,2,2-trifluoroacetic acid

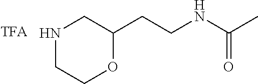

Tert-butyl 2-(2-acetamidoethyl)morpholine-4-carboxylate (350 mg, 1.2852 mmol) was solubilized in 15 ml of DCM. The solution was cooled to 0° C. and TFA (1.5 ml) was added.

After 2 h stirring at room temperature, the reaction mixture was concentrated under reduce pressure to give the title compound as a colourless oil (631 mg), which was used without further purification in the next step.

1H NMR (400 MHz, DMSO-d6, 30° C.) δ ppm 1.45-1.67 (m, 2H), 1.79 (s, 3H), 2.65-2.81 (m, 1H), 2.89-3.10 (m, 2H), 3.11-3.25 (m, 3H), 3.55-3.72 (m, 2H), 3.95 (dd, J=12.6, 3.5 Hz, 1H), 7.89 (br t, J=5.5 Hz, 1H), 8.77-9.21 (m, 2H)

### 2-[6-[(2R)-2-(2-hydroxyethyl)morpholin-4-yl]pyridazin-3-yl]-3,5-dimethyl-phenol (Example 277) and 2-[6-[(2S)-2-(2-hydroxyethyl)morpholin-4-yl]pyridazin-3-yl]-3,5-dimethyl-phenol (Example 278)

In a microwave vial, under Ar, to a solution of 2-(6-chloropyridazin-3-yl)-3-methyl-5-(trifluoromethyl)phenol (150 mg, 0.5197 mmol) in 6 ml of 1-Butanol were added N-(2-morpholin-2-ylethyl)acetamide;2,2,2-trifluoroacetic acid (520 mg 1.2991 mmol) and Et₃N (362 μl, 2.5983 mmol). The mixture was heated at 160° C. under microwave irradiation for 2 h. After cooling to rt, the reaction mixture was diluted with AcOEt, the organic layer was washed with water and brine, dried over MgSO₄, concentrated under reduced pressure. The residue was purified by flash chromatography on silica gel using DCM/MeOH (100/0 to 95/6) to give 140 mg of N-[2-[4-[6-[2-hydroxy-6-methyl-4-(trifluoromethyl) phenyl]pyridazin-3-yl]morpholin-2-yl]ethyl]acetamide.

The 2 enantiomers of N-[2-[4-[6-[2-hydroxy-6-methyl-4-(trifluoromethyl) phenyl]pyridazin-3-yl]morpholin-2-yl]ethyl]acetamide were separated by chiral phase LC (Column CHIRALPAK® IC 20 μm, 76.5×400 mm, Flow rate 400 ml/min, eluent n-heptane/ethanol (75/25)+0.1% TEA), to give both enantiomers, first eluting Rt=8.9 min, Example 277 (55 mg) and second eluting Rt=11.9 min, Example 278 (53 mg).

The absolute configuration was unknown for the two compounds.

### Example 203: 3,5-dichloro-2-(6-thiomorpholinopyridazin-3-yl)phenol

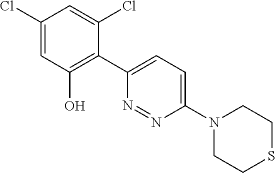 [SMILES: Oc1cc(Cl)cc(Cl)c1-c1ccc(N2CCSCC2)nn1]

A solution of 3,5-dichloro-2-(6-chloropyridazin-3-yl)phenol (Int 58.5-0.911 mmol, 251 mg), thiomorpholine (1.37 mmol, 141 mg) and triethylamine (1.09 mmol, 152 μl) in 9 ml of n-butanol was stirred at 120° C. for 18 h. The mixture was cooled down, concentrated under reduced pressure and the residue was diluted with 50 ml of dichloromethane then washed successively with 5 ml of a saturated aqueous sodium bicarbonate solution and 5 ml of brine. The organic layer was dried over Na₂SO₄, filtered and concentrated under reduced pressure.

The residue was purified by flash chromatography on silica gel eluting with a gradient of 0 to 5% of EtOAc in toluene to give the title compound (115 mg, 37% yield) as a white solid.

### Example 317: 2-[6-(4-ethyl-1,11-dioxa-4,8-diazaspiro[5.6]dodecan-8-yl)pyridazin-3-yl]-3,5-dimethyl-phenol

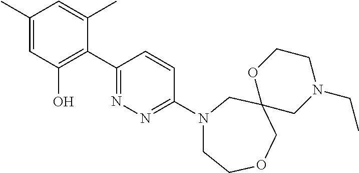 [SMILES: CCN1CCOC2(COCCN(c3ccc(-c4c(C)cc(C)cc4O)nn3)C2)C1]

### Int 317.1: tert-butyl 4-ethyl-1,11-dioxa-4,8-diazaspiro[5.6]dodecane-8-carboxylate

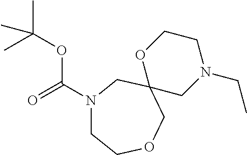 [SMILES: CCN1CCOC2(COCCN(C(=O)OC(C)(C)C)C2)C1]

To a stirred solution of tert-butyl 1,11-dioxa-4,8-diazaspiro[5.6]dodecane-8-carboxylate (150 mg, 5.51 mmol) in 5 ml of acetonitrile were added K₂CO₃ (152 mg, 1.10 mmol) and iodoethane (0.0576 mL, 0.716 mmol). The reaction mixture was stirred at rt for 18 h. The reaction was quenched with water and extracted with DCM. The organic layer was dried over MgSO₄, filtered and concentrated under reduced pressure to give the title compound (130 mg, 79%) which was used without further purification in the next step.

1H NMR (400 MHz, DMSO-d6) δ (ppm): 3.78-3.37 (m, 11H), 2.40-2.06 (m, 5H), 1.40 (s, 9H), 0.98 (t, J=7.1 Hz, 3H).

### Int 317.2: 4-ethyl-1,11-dioxa-4,8-diazaspiro[5.6]dodecane;2,2,2-trifluoroacetic acid

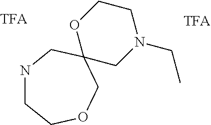

At 0° C., to a stirred solution of tert-butyl 4-ethyl-1,11-dioxa-4,8-diazaspiro[5.6]dodecane-8-carboxylate (80 mg, 0.600 mmol) in 5 ml of dichloromethane was added TFA (0.684 g, 0.006.0 mmol). The reaction mixture was stirred at 30° C. for 4 h. The reaction mixture was concentrated under reduced pressure to give the title compound (270 mg, quant. Yield: 105%), which was used without further purification in the next step.

1H NMR (500 MHz, DMSO-d6) δ (ppm): 5.59-5.31 (m, 1H), 3.78-3.60 (m, 2H), 3.62-3.28 (m, 4H), 3.16-3.04 (m, 1H), 2.98-2.89 (m, 1H), 2.90-2.71 (m, 2H), 2.37-2.31 (m, 1H), 2.30-2.21 (m, 3H), 2.22-2.15 (m, 1H), 2.03 (d, J=11.4 Hz, 1H), 0.97 (t, J=7.2 Hz, 3H).

### Int 317.3: 2-(6-chloropyridazin-3-yl)-3,5-dimethyl-phenol

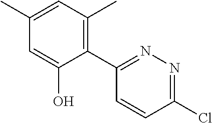 [SMILES: Cc1cc(C)c(-c2ccc(Cl)nn2)c(O)c1]

In a microwave vial, under N₂, to a mixture of (2-hydroxy-4,6-dimethyl-phenyl)boronic acid (730 mg, 4.40 mmol) in 16 ml of 1,4-dioxane were added 3-bromo-6-chloro-pyridazine (1.00 g, 5.16 mmol) and K₃PO₄ (3.30 g, 15.5 mmol) in 4 ml of water. The mixture was purged with N₂. Pd₁₁₈ (370 mg, 0.568 mmol) was added, and the mixture was purged again with N₂. The mixture was then heated at 90° C. for 2.5 h. After cooling down to rt, the mixture was concentrated under reduced pressure. The residue was purified by flash chromatography on silica gel using heptane/EtOAc (100/0 to 50/50, then 30/70) to give the title compound as an orange solid (180.5 mg, 15%).

LCMS (Method S): Rt=1.08; MS m/z [M−H]⁺=235.1

### 2-[6-(4-ethyl-1,11-dioxa-4,8-diazaspiro[5.6]dodecan-8-yl)pyridazin-3-yl]-3,5-dimethyl-phenol (Example 317)

To a stirred solution of 2-(6-chloropyridazin-3-yl)-3,5-dimethyl-phenol (Int 317.3-20.0 mg, 0.0852 mmol) in 1 ml of tetrahydrofuran were added 4-ethyl-1,11-dioxa-4,8-diazaspiro[5.6]dodecane;2,2,2-trifluoroacetic acid (Int 317.2-36.5 mg, 0.0852 mmol), LiHMDS (1M in THF) (0.460 mL, 0.460 mmol) and BrettPhos Pd G3 (3.86 mg, 0.00426 mmol) under inert atmosphere. The reaction mixture was stirred at 90° C. for 2.5 h. The reaction mixture was cooled down to rt, filtered and concentrated under reduced pressure. The residue was purified by flash chromatography on silica gel using DCM/EtOAc (100/0 to 95/5) to give 8.5 mg of a colorless oil. This oil was purified by reverse phase Column YMC—Actus Triart Prep C18-S 150*30 mm 5 μm Flow rate 40 ml/min-Focused gradient MeCN/aq·NH₄HCO₃ 0.2% pH=7.9 Focused gradient from 35/65 to 75/25, to give after lyophilization, the title compound (3 mg, 6%).

### Examples 329 and 330: (6R)-4-[6-(2-hydroxy-4,6-dimethyl-phenyl)pyridazin-3-yl]-6-(hydroxymethyl)piperazin-2-one and (6S)-4-[6-(2-hydroxy-4,6-dimethyl-phenyl)pyridazin-3-yl]-6-(hydroxymethyl)piperazin-2-one

### Int 329.1: 2-(6-chloropyridazin-3-yl)-3,5-dimethyl-phenol

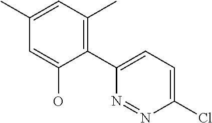 [SMILES: Cc1cc(C)c(-c2ccc(Cl)nn2)c(O)c1]

Under Ar, to a solution of (2-hydroxy-4,6-dimethylphenyl)boronic acid (12.05 mmol, 2 g) and 3,6-dichloropyridazine (12.05 mmol, 1.79 g) in 32 ml of 1,2-dimethoxyethane were added sodium carbonate (30.12 mmol, 3.19 g) and 8 ml of water. The solution was purged with Ar, then Pd(PPh₃)₄ (0.60 mmol, 696 mg) was added. The reaction mixture was heated under reflux for 4 h. After cooling down to rt, the solution was concentrated under reduced pressure. 42 ml of EtOAc was added and the resulting mixture was filtered. The solid was taken up in a EtOAc/CH₂Cl₂ mixture, to give after trituration and filtration, the title compound as a pale brown solid (866 mg). Besides, to the filtrate, EtOAc was added, and the organic layer was washed with water, dried over Na₂SO₄, filtered and concentrated under reduced pressure. The residue was triturated in CH₂Cl₂, then filtered to afford the title compound as a pale brown solid (858 mg). Overall, the title compound was obtained as a pale brown solid (1.57 g, 56% yield).

LCMS (Method U): Rt=1.36 min; MS m/z [M+H]+=235

### Int 329.2: 4-[6-(2-hydroxy-4,6-dimethyl-phenyl)pyridazin-3-yl]-6-(hydroxymethyl) piperazin-2-one

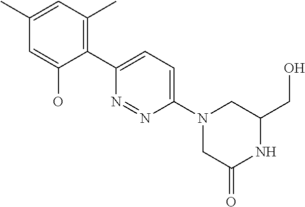 [SMILES: Cc1cc(C)c(-c2ccc(N3CC(=O)NC(CO)C3)nn2)c(O)c1]

In a microwave vial, under Ar, to a suspension of 2-(6-chloropyridazin-3-yl)-3,5-dimethyl-phenol (0.43 mmol, 100 mg) in 1 ml of 1-butanol was added 6-(hydroxymethyl)piperazin-2-one hydrochloride (0.64 mmol, 106 mg), diisopropylethylamine (0.85 mmol, 0.15 ml) and the resulting mixture was heated at 150° C. under microwave irradiation for 1 h30. After cooling down to rt, the reaction mixture was concentrated under reduced pressure. The residue was purified by flash chromatography on silica gel using DCM/MeOH/ACN (100/0/0 to 80/10/10) to give the title compound as a yellow solid (61 mg, 44% yield).

LCMS (Method G): Rt=1.69; MS m/z [M+H]+=329

### (6R)-4-[6-(2-hydroxy-4,6-dimethyl-phenyl)pyridazin-3-yl]-6-(hydroxymethyl) piperazin-2-one and (6S)-4-[6-(2-hydroxy-4,6-dimethyl-phenyl)pyridazin-3-yl]-6-(hydroxymethyl)piperazin-2-one (Examples 329 and 330)

The 2 enantiomers of 4-[6-(2-hydroxy-4,6-dimethyl-phenyl)pyridazin-3-yl]-6-(hydroxymethyl)piperazin-2-one were separated by chiral phase LC (Column Chiralpak® IC 5 μm, 250×30 mm, Flow rate 40 ml/min, eluent (n-heptane/ethanol 60/40)+0.10% TEA, to give both enantiomers, first eluting Rt=10.3 min, Example 329 (15.8 mg, yield 26%) and second eluting Rt=14.4 min, Example 330 (14.5 mg, yield 24%). The absolute configurations cannot be assigned at this stage.

Chiral separation of compounds Examples 419 and 420 were realized analogously to Example 329 and 330.

### Example 346: 3-methyl-2-[6-[rac-(3aS,6aS)-2,3,3a,5,6,6a-hexahydro-1H-pyrrolo[3,2-b]pyrrol-4-yl]pyridazin-3-yl]-5-(trifluoromethyl)phenol;hydrochloride

### Int 346.1: tert-butyl rac-(3aS,6aS)-1-[6-[2-hydroxy-6-methyl-4-(trifluoromethyl) phenyl]pyridazin-3-yl]-2,3,3a,5,6,6a-hexahydropyrrolo[3,2-b]pyrrole-4-carboxylate

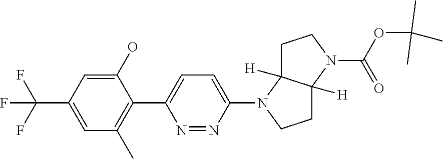 [SMILES: Cc1cc(C(F)(F)F)cc(O)c1-c1ccc(N2CCC3C2CCN3C(=O)OC(C)(C)C)nn1]

To a solution of 2-(6-chloropyridazin-3-yl)-3-methyl-5-(trifluoromethyl)phenol (100 mg, 0.3464 mmol) in 2 ml of 1-Butanol was added tert-butyl cis-hexahydropyrrolo[3,2-B]pyrrole-1(2H)-carboxylate (147 mg, 0.6929 mmol) and Triethylamine (0.121 ml, 08661 mmol). The mixture was heated at 160° C. for 2 h under microwave irradiation. After cooling down to rt, the reaction mixture was diluted with AcOEt, washed with water and brine, dried over MgSO₄, filtered and concentrated under reduced pressure. The residue was purified by flash chromatography on silica gel eluting with a gradient of DCM/MeOH (from 100/0 to 95/5) to give the title compound as a yellow solid (62 mg, 38% yield).

LCMS (Method G): Rt=1.01; MS m/z [M+H]⁺=465

### 3-methyl-2-[6-[rac-(3aS,6aS)-2,3,3a,5,6,6a-hexahydro-1H-pyrrolo[3,2-b]pyrrol-4-yl]pyridazin-3-yl]-5-(trifluoromethyl)phenol;hydrochloride (Example 346)

To a solution of tert-butyl rac-(3aS,6aS)-1-[6-[2-hydroxy-6-methyl-4-(trifluoromethyl)phenyl]pyridazin-3-yl]-2,3,3a,5,6,6a-hexahydropyrrolo[3,2-b]pyrrole-4-carboxylate (62 mg, 0.1335 mmol) in 4 ml of DCM was added a solution of HCl 4N in dioxane (0.33 ml, 1.3348 mmol) and mixture was heated at 50° C. for 1 h and stirred at RT overnight. Additional HCl 4N in dioxane (0.40 ml 1.6179 mmol) was added and the reaction mixture was heated at 50° C. for 3 h. After cooling down to rt, the mixture was concentrated under reduced pressure. The residue was crystallized in acetone, filtered, and washed with acetone to give the title compound as a cream solid (36 mg, 67% yield).

### Example 410: 3,5-dimethyl-2-[6-(3-methyl-3,6-diazabicyclo[3.2.2]nonan-6-yl)pyridazin-3-yl]phenol

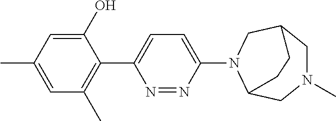

### Int 410.1: tert-butyl 3-methyl-3,6-diazabicyclo[3.2.2]nonane-6-carboxylate

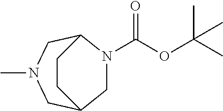 [SMILES: CN1CC2CCC(C1)N(C(=O)OC(C)(C)C)C2]

To a solution of tert-butyl 3,6-diazabicyclo[3.2.2]nonane-6-carboxylate (400 mg, 1.7674 mmol) in 12 ml of THF was added by portions sodium hydride 60% (78 mg, 1.9442 mmol).

After stirring 30 min, Iodomethane (276 mg, 1.9442 mmol) was added, and the mixture was stirred at rt overnight. The mixture was concentrated under reduce pressure. The residue was purified by flash chromatography on silica gel eluting with a gradient of DCM/MeOH (100/0 to 97/3) to give the title compound as a yellow oil (450 mg), which was used without further purification in the next step.

### Int 410.2: 3-methyl-3,6-diazabicyclo[3.2.2]nonane;2,2,2-trifluoroacetic acid

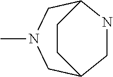

To a solution of tert-butyl 3-methyl-3,6-diazabicyclo[3.2.2]nonane-6-carboxylate (450 mg, 1.8723 mmol) in 20 ml of DCM was added 2 ml of TFA. The reaction was stirred for 16 h. The reaction was concentrated under reduce pressure and the residue obtained was used without further purification in the next step.

### 3,5-dimethyl-2-[6-(3-methyl-3,6-diazabicyclo[3.2.2]nonan-6-yl)pyridazin-3-yl]phenol (Example 410)

In a microwave vial, to a solution of 2-(6-chloropyridazin-3-yl)-3,5-dimethyl-phenol (100 mg, 0.4261 mmol) in 2 ml of 1-Butanol was added 3-methyl-3,6-diazabicyclo[3.2.2]nonane;2,2,2-trifluoroacetic acid (313 mg, 0.8522 mmol) and Triethylamine (0.148 ml, 1.0653 mmol). The mixture was heated at 160° C. under microwave irradiation for 2 h. Additional 3-methyl-3,6-diazabicyclo[3.2.2]nonane;2,2,2-trifluoroacetic acid (100 mg, 0.2723 mmol) and Triethylamine (0.200 ml, 1.4396 mmol) were added and the mixture was heated for 1 h30 at 160° C. After cooling down to rt, the mixture was diluted with AcOEt, washed with water and brine, dried over MgSO₄, concentrated under reduced pressure. The residue was purified by flash chromatography on silica gel eluting with a gradient of DCM/MeOH (100/0 to 90/10) to give, after crystallization in pentane, the title compound as a white solid (10 mg, 7% yield).

Compound Examples 417 was synthesized analogously to Example 410.

### Example 372: (4S)-1-[5-(difluoromethyl)-6-[2-hydroxy-6-methyl-4-(trifluoromethyl) phenyl] pyridazin-3-yl]-4-hydroxy-pyrrolidin-2-one

### Int 372.1: 2-[6-chloro-4-(difluoromethyl)pyridazin-3-yl]-3-methyl-5-(trifluoromethyl) phenol

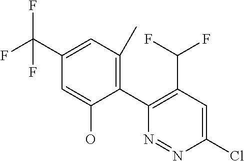 [SMILES: Cc1cc(C(F)(F)F)cc(O)c1-c1nnc(Cl)cc1C(F)F]

In a microwave vial, under Ar, to a solution of 3-methyl-2-(4,4,5,5-tetramethyl-1,3,2-dioxaborolan-2-yl)-5-(trifluoromethyl)phenol (2.48 mmol, 0.75 g) and 3,6-dichloro-4-(difluoromethyl)pyridazine (2.48 mmol, 0.49 g) in 12 ml of 1,2-dimethoxyethane were added sodium carbonate (6.20 mmol, 0.66 g), 3 ml of water and Pd(PPh₃)₄ (0.12 mmol, 143 mg). The reaction mixture was purged with Ar, then heated at 100° C. under microwave irradiation for 1 h. After cooling down to rt, EtOAc was added, and the resulting mixture was filtered. The filtrate was concentrated under reduced pressure. The residue was purified by flash chromatography on silica gel using DCM/MeOH/ACN (100/0/0 to 96/2/2) to give the title compound as a brown solid (88 mg, 10% yield).

LCMS (Method B): Rt=1.64; MS m/z [M+H]+=339

### (4S)-1-[5-(difluoromethyl)-6-[2-hydroxy-6-methyl-4-(trifluoromethyl)phenyl]pyridazin-3-yl]-4-hydroxy-pyrrolidin-2-one (Example 372)

In a microwave vial, under Ar, a suspension of 2-[6-chloro-4-(difluoromethyl)pyridazin-3-yl]-3-methyl-5-(trifluoromethyl)phenol (0.24 mmol, 82 mg), (S)-4-hydroxy-2-pyrrolidinone (0.29 mmol, 29 mg), potassium phosphate tribasic (0.48 mmol, 103 mg), 1-1′-bis(diphenylphosphino)ferrocene (0.07 mmol, 40 mg), palladium acetate (0.02 mmol, 5 mg) in 1.2 ml of 1,4-dioxane was heated at 110° C. under microwave irradiation for 2 h. After cooling down to rt, EtOAc and CH₂Cl₂ were added, and the resulting mixture was filtered. The filtrate was concentrated under reduced pressure. The residue was purified by flash chromatography on silica gel using DCM/MeOH/ACN (100/0/0 to 90/5/5) to give the title compound as a brown solid (26 mg, 27% yield).

### Example 399 and 400: 3,5-dichloro-2-[6-(1,1-dioxo-1,4-thiazinan-4-yl)pyridazin-3-yl]phenol and 3,5-dichloro-2-[6-(1-oxo-1,4-thiazinan-4-yl)pyridazin-3-yl]phenol

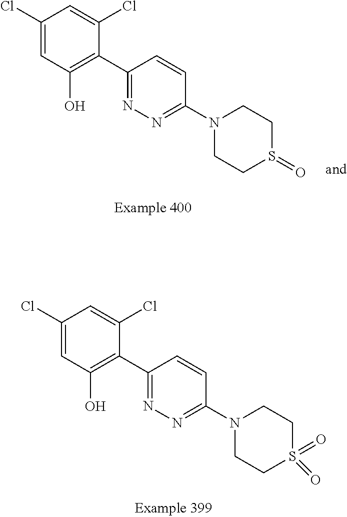 [SMILES: O=S1CCN(c2ccc(-c3c(O)cc(Cl)cc3Cl)nn2)CC1 ; O=S1(=O)CCN(c2ccc(-c3c(O)cc(Cl)cc3Cl)nn2)CC1]

At 0° C., to a solution of 3,5-dichloro-2-(6-thiomorpholinopyridazin-3-yl)phenol (Example 203-0.14 mmol, 48 mg) in 10 ml of DCM was added 3-chloroperoxybenzoic acid (0.18 mmol, 31 mg). The mixture was stirred at 0° C. for 25 min, then diluted with 20 ml of DCM. The organic layer was washed with 20 ml of a saturated aqueous sodium bicarbonate solution and 20 ml of brine, then dried over Na₂SO₄, filtered and concentrated under reduced pressure. The residue was purified by flash chromatography on silica gel eluting with a gradient of 0 to 5% of MeOH in DCM to give successively Example 399 (26 mg, 49% yield) as a pale yellow solid and Example 400 (25 mg, 50% yield) as a white solid.

### Examples 466 and 467: (4aR,6R,7aS)-4-[6-(2-hydroxy-4,6-dimethyl-phenyl)pyridazin-3-yl]-3,4a,5,6,7,7a-hexahydro-2H-cyclopenta[b][1,4]oxazin-6-ol and (4aS,6S,7aR)-4-[6-(2-hydroxy-4,6-dimethyl-phenyl)pyridazin-3-yl]-3,4a,5,6,7,7a-hexahydro-2H-cyclopenta[b][1,4]oxazin-6-ol

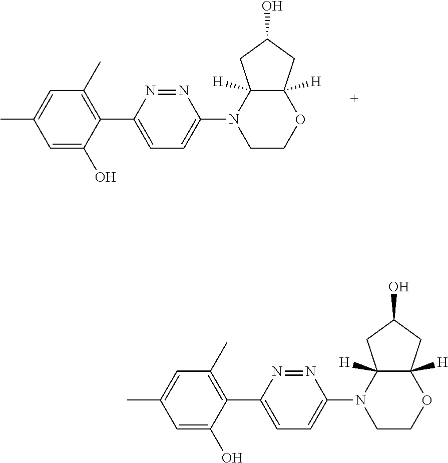 [SMILES: Cc1cc(C)c(-c2ccc(N3CCO[C@@H]4C[C@@H](O)C[C@@H]43)nn2)c(O)c1 ; Cc1cc(C)c(-c2ccc(N3CCO[C@H]4C[C@H](O)C[C@H]43)nn2)c(O)c1]

### Int 466.1: 1-(2-hydroxy-4,6-dimethyl-phenyl)ethanone

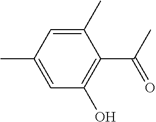

At rt, under Ar, to 3, 5-dimethylphenol (24.4 g, 200 mmol) was slowly added TiCl₄ (24.1 mL, 220 mmol). The reacting mixture was stirred at rt. When gas evolution ceased, acetyl chloride (23.5 g, 300 mmol) was added. The resulting mixture was stirred at room temperature for 15 minutes, then warmed to 120° C. for 1 h. After cooling down to rt, the resulting mixture was diluted with methylene chloride (250 mL) and quenched with water (150 mL). The organic layer was washed twice with water, dried over Na₂SO₄, filtered and concentrated under reduced pressure. The residue was purified by flash chromatography on silica gel eluting with petroleum ether/EA (100/0 to 95/5) to give the title compound as a light yellow solid (29.5 g, yield: 90%).

1H NMR (400 MHz, CDCl₃) δ: 12.68 (s, 1H), 6.65 (s, 1H), 6.54 (s, 1H), 2.64 (s, 3H), 2.56 (s, 3H), 2.27 (s, 3H) ppm.

### Int 466.2: 6-(2-hydroxy-4,6-dimethyl-phenyl)pyridazin-3-ol

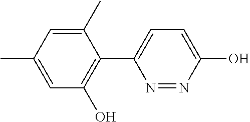 [SMILES: Cc1cc(C)c(-c2ccc(O)nn2)c(O)c1]

In an ice-bath, a solution of aqueous potassium hydroxide (20%, w/v) was added dropwise to an aqueous solution of glyoxylic acid (50%, 27.1 g, 183 mmol) till the pH reach 9. Then was added dropwise while cooling a solution of 1-(2-hydroxy-4,6-dimethyl-phenyl)ethanone (Int 466.1, 30 g, 183 mmol) in an aqueous potassium hydroxide solution (20% w/v, 10.3 g of KOH). The resulting mixture was stirred for 2.5 h at rt, then cooled to 0° C. and acidified with acetic acid to pH=8. The solution was extracted twice with dichloromethane. Acetic acid was added to the aqueous solution till pH=4. Then ammonia solution was added till pH=8. Hydrazine hydrate 85% (11.8 g, 20.1 mmol) was added, the reaction mixture was inerted under Ar, then warmed for 2.5 h at 100° C. After cooling down to rt, the solid was filtered off, washed with water and dried over P₂O₅ to give the title compound as white solid (12.7 g, 32.1% yield).

LCMS (Method Z5): Rt=1.56; MS m/z [M+H]⁺=217

### Int 466.3: 2-(6-chloropyridazin-3-yl)-3,5-dimethyl-phenol

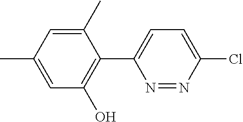 [SMILES: Cc1cc(C)c(-c2ccc(Cl)nn2)c(O)c1]

Under Ar, in an ice bath, to a solution of phosphorus oxychloride (44.7 g, 291 mmol) in 40 ml of DMF was added quickly by portions 6-(2-hydroxy-4,6-dimethyl-phenyl)pyridazin-3-ol (Int 466.2-12.6 g, 58.3 mmol). The solution was heated to 85° C. for 18 h. After cooling down to rt, the reacting mixture was poured slowly into ice-cold water and extracted twice with ethyl acetate. The organic layers were dried over Na₂SO₄, filtered and concentrated under reduced pressure. The residue was purified by flash chromatography on silica gel eluting with petroleum ether/EA (100/0 to 95/5) to give the title compound as a yellow solid (7.8 g, 57% yield).

1HNMR (500 MHz, DMSO-d₆) δ=9.54 (s, 1H), 7.92 (d, J=8.5 Hz, 1H), 7.74 (d, J=9.0 Hz, 1H), 6.63 (d, J=4.0 Hz, 2H), 2.24 (s, 3H), 2.02 (s, 3H) ppm.

### Int 466.4: 3-(2-benzyloxy-4,6-dimethyl-phenyl)-6-chloro-pyridazine

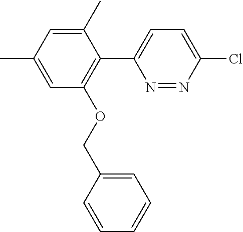 [SMILES: Cc1cc(C)c(-c2ccc(Cl)nn2)c(OCc2ccccc2)c1]

To a solution of 2-(6-chloropyridazin-3-yl)-3,5-dimethyl-phenol (Int 466.3-3.0 g, 12.8 mmol) in 60 ml of DMF was added bromomethylbenzene (2.62 g, 15.3 mmol) and K₂CO₃ (3.53 g, 25.6 mmol). The solution was stirred at room temperature overnight. The resulting solution was poured into water and extracted with Ethyl acetate (3 times). The combined organic layers were washed by brine, dried over Na₂SO₄, filtered and concentrated under reduced pressure. The residue was purified by flash chromatography on silica gel eluting with petroleum ether/EA (100/0 to 0/100) to give the title compound as an oil (3.80 g, yield 91.5%).

LCMS (Method Z6): Rt=2.15; MS m/z [M+H]⁺=325

### Int 466.5: cyclopent-3-en-1-yloxymethylbenzene

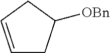 [SMILES: C1=CCC(OCc2ccccc2)C1]

At 0° C., to a solution of cyclopent-3-en-1-ol (10.0 g, 0.119 mol) in 100 ml of toluene was slowly added NaH (60%, 7.13 g, 0.178 mol). The mixture was stirred at 0° C. for 1 h and benzyl bromide (22.4 g, 0.131 mol) was added. The reaction mixture was stirred at rt for 18 h. The resulting mixture was poured into ice water (300 mL) and extracted with DCM (100 mL×3). The organic layer was washed by brine, dried over Na₂SO₄, filtered and concentrated under reduced pressure. The residue was purified by flash chromatography on silica gel eluting with petroleum ether 100% to give the title compound as an oil (14.8 g, 71.5% yield).

LCMS (Method Z4): Rt=2.11; MS m/z [M+18]⁺=192.1

### Int 466.6a and Int 466.6b: (1R,3s,5S)-3-(benzyloxy)-6-oxabicyclo[3.1.0]hexane and (1R,3r,5S)-3-(benzyloxy)-6-oxabicyclo[3.1.0]hexane

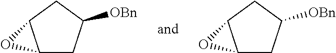 [SMILES: c1ccc(CO[C@H]2C[C@@H]3O[C@@H]3C2)cc1 ; c1ccc(CO[C@@H]2C[C@@H]3O[C@@H]3C2)cc1]

At 0° C., to a solution of cyclopent-3-en-1-yloxymethylbenzene (14.8 g, 84.9 mmol) in 200 ml of DCM was added m-CPBA (29.3 g, 170.0 mmol). The solution was stirred at rt overnight. The resulting mixture was quenched with saturated Na₂SO₃ solution and adjusted to pH=8˜ 9 with saturated Na₂CO₃ solution. The resulting mixture was extracted with DCM (3 times). The pooled organic layers were washed by brine, dried over Na₂SO₄, filtered and concentrated under reduced pressure. The residue was purified by flash chromatography on silica gel eluting with petroleum ether/EA (100/0 to 0/100) to give (1R,3s,5S)-3-(benzyloxy)-6-oxabicyclo[3.1.0]hexane as an oil (6.0 g, 37.1% yield) and (1R,3r,5S)-3-(benzyloxy)-6-oxabicyclo[3.1.0]hexane as an oil (6.7 g, 41.5% yield).

LCMS:
Int 466.6a (Method Z4): Rt=1.69; MS m/z [M+H]⁺=191.1
Int 466.6b (Method Z4): Rt=1.55; MS m/z [M+H]⁺=191.1

### Int 466.7: rac-(1R,2R,4R)-2-amino-4-benzyloxy-cyclopentanol

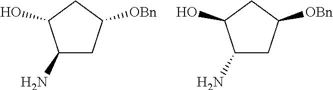 [SMILES: N[C@@H]1C[C@@H](OCc2ccccc2)C[C@H]1O ; N[C@H]1C[C@H](OCc2ccccc2)C[C@@H]1O]

In a vial, to (1R,3r,5S)-3-(benzyloxy)-6-oxabicyclo[3.1.0]hexane (Int 466.6b, 4.70 g, 24.7 mmol) was added 60 ml of NH₃·H₂O (28% in water). The vial was sealed and stirred at 60° C. overnight. The reaction mixture was concentrated under reduced pressure to give the title compound as a solid (5.12 g) which was used without further purification in the next step.

LCMS (Method Z4): Rt=1.11; MS m/z [M+H]⁺=208.1

### Int 466.8: tert-butyl N-[rac-(1R,2R,4R)-4-benzyloxy-2-hydroxy-cyclopentyl]carbamate

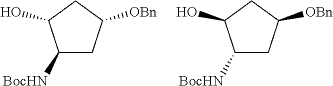 [SMILES: CC(C)(C)OC(=O)N[C@@H]1C[C@@H](OCc2ccccc2)C[C@H]1O ; CC(C)(C)OC(=O)N[C@H]1C[C@H](OCc2ccccc2)C[C@@H]1O]

To a solution of rac-(1R,2R,4R)-2-amino-4-benzyloxy-cyclopentanol (5.12 g, 24.7 mmol) in 50 ml of methanol was added Boc₂O (8.09 g, 37.1 mmol) and TEA (5.16 mL, 37.1 mmol). The resulting mixture was stirred overnight at rt. The reaction mixture was concentrated under reduced pressure. The residue was purified by flash chromatography on silica gel eluting with petroleum ether/EA (100/0 to 0/100) to give the title compound as a solid (4.59 g, 60.4% two steps yield).

LCMS (Method Z5): Rt=1.84; MS m/z [M+H]⁺=330.4

### Int 466.9: [rac-(1S,2R,4R)-4-benzyloxy-2-(tert-butoxycarbonylamino)cyclopentyl]benzoate

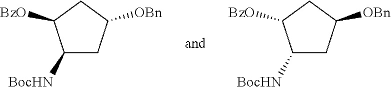

At 0° C., to a solution of PPh₃ (5.87 g, 22.4 mmol) in 50 ml of anhydrous THF was added DIAD (4.41 mL, 22.4 mmol). The solution was stirred at 0° C. for 20 min. Then were added tert-butyl N-[rac-(1R,2R,4R)-4-benzyloxy-2-hydroxy-cyclopentyl]carbamate (Int 466.8; 4.59 g, 14.9 mmol) and benzoic acid (2.19 g, 17.9 mmol), and the reaction was stirred at room temperature for 16 h. The reaction mixture was quenched with water, extracted with ethyl acetate (3 times). The combined organic layers were washed with brine, dried over anhydrous Na₂SO₄, filtered and concentrated under reduced pressure. The residue was purified by flash chromatography on silica gel eluting with petroleum ether/EA (100/0 to 0/100) to give the title compound as a solid (5.20 g, 74.4% yield).

LCMS (Method Z5): Rt=2.14; MS m/z [M-boc]⁺=312.4

### Int 466.10: tert-butyl N-[rac-(1R,2S,4R)-4-benzyloxy-2-hydroxy-cyclopentyl]carbamate

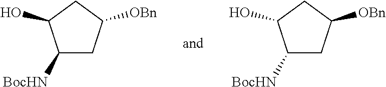 [SMILES: CC(C)(C)OC(=O)N[C@@H]1C[C@@H](OCc2ccccc2)C[C@@H]1O ; CC(C)(C)OC(=O)N[C@H]1C[C@H](OCc2ccccc2)C[C@H]1O]

To a solution of [rac-(1S,2R,4R)-4-benzyloxy-2-(tert-butoxycarbonylamino)cyclopentyl]benzoate (5.20 g, 11.1 mmol) in 100 ml of ethanol and 50 ml of water was added K₂CO₃ (2.30 g, 16.7 mmol). The solution was stirred at rt overnight. The reaction mixture was concentrated under reduced pressure. To the residue was added EA and water. The aqueous layer was extracted twice with EA. The combined organic layers were washed with brine, dried over anhydrous Na₂SO₄, filtered and concentrated under reduced pressure. The residue was purified by flash chromatography on silica gel eluting with petroleum ether/acetone (100/0 to 0/100) to give the title compound as a solid (3.07 g, yield 88.9%).

LCMS (Method Z5): Rt=1.87; MS m/z [M+Na]⁺=330.4

### Int 466.11: rac-(1S,2R,4R)-2-amino-4-benzyloxy-cyclopentanol;hydrochloride

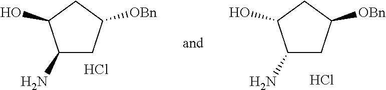 [SMILES: N[C@@H]1C[C@@H](OCc2ccccc2)C[C@@H]1O ; Cl ; N[C@H]1C[C@H](OCc2ccccc2)C[C@H]1O ; Cl]

To a solution of tert-butyl N-[rac-(1R,2S,4R)-4-benzyloxy-2-hydroxy-cyclopentyl]carbamate (3.07 g, 9.53 mmol) in 30 ml of DCM was added 5 ml of HCl (4 M in 1,4-dioxane). The solution was stirred at rt for 2 h. The reaction mixture was concentrated under reduced pressure to give the title compound as solid (2.56 g), which was used without further purification in the next step.

LCMS (Method Z5): Rt=1.37; MS m/z [M+H]⁺=208.4

### Int 466.12: 4-nitro-N-[rac-(1R,2S,4R)-4-benzyloxy-2-hydroxy-cyclopentyl]benzenesulfonamide

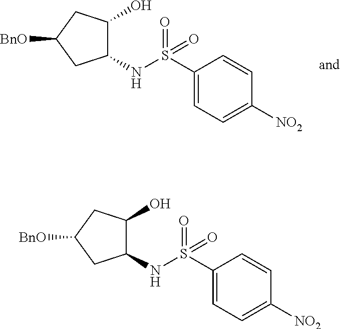 [SMILES: O=[N+]([O-])c1ccc(S(=O)(=O)N[C@@H]2C[C@@H](OCc3ccccc3)C[C@@H]2O)cc1 ; O=[N+]([O-])c1ccc(S(=O)(=O)N[C@H]2C[C@H](OCc3ccccc3)C[C@H]2O)cc1]

To a solution of rac-(1S,2R,4R)-2-amino-4-benzyloxy-cyclopentanol;hydrochloride (2.56 g, 10.5 mmol) in 30 ml of anhydrous DCM was added 4-nitrobenzenesulfonyl chloride (2.56 g, 11.6 mmol) and TEA (4.39 mL, 31.5 mmol). The mixture was stirred for 2 h at rt. The reaction mixture was quenched with water and extracted with DCM (3 times). The combined organic layers were washed with brine, dried over anhydrous Na₂SO₄, filtered and concentrated under reduced pressure. The residue was purified by flash chromatography on silica gel eluting with petroleum ether/EA (100/0 to 0/100) to give the title compound as a solid (3.07 g, two steps 78% yield).

LCMS (Method Z5): Rt=1.84; MS m/z [M+H]⁺=393.3

### Int 466.13: rac-(4aR,6R,7aS)-6-benzyloxy-4-(4-nitrophenyl)sulfonyl-3,4a,5,6,7,7a-hexahydro-2H-cyclopenta[b][1,4]oxazine

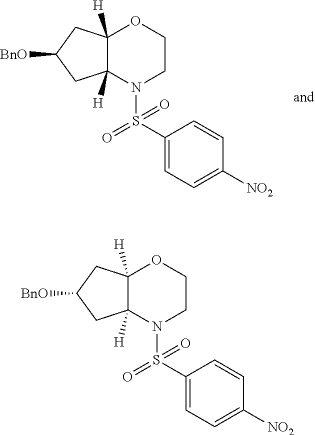 [SMILES: O=[N+]([O-])c1ccc(S(=O)(=O)N2CCO[C@H]3C[C@H](OCc4ccccc4)C[C@H]32)cc1 ; O=[N+]([O-])c1ccc(S(=O)(=O)N2CCO[C@@H]3C[C@@H](OCc4ccccc4)C[C@@H]32)cc1]

At 0° C., to a solution of 4-nitro-N-[rac-(1R,2S,4R)-4-benzyloxy-2-hydroxy-cyclopentyl]benzenesulfonamide (3.07 g, 6.92 mmol) in 400 ml of anhydrous DCM was added NaH (60%, 1.50 g, 37.4 mmol). After stirring for 10 minutes was added 2-bromoethyl(diphenyl)sulfonium; trifluoromethanesulfonate (8.29 g, 18.7 mmol). After stirring overnight at rt, TEA (2.51 mL) was added. The reaction was stirred at rt overnight. The reaction mixture was quenched with saturated NH₄Cl and extracted with DCM (3 times). The combined organic layers were washed by brine, dried over anhydrous Na₂SO₄, filtered and concentrated under reduced pressure. The residue was purified by flash chromatography on silica gel eluting with petroleum ether/EA (100/0 to 0/100) to give the title compound as a solid (1.20 g, 39.9% yield).

LCMS (Method Z5): Rt=1.93; [M+H]⁺=419.3

### Int 466.14: rac-(4aR,6R,7aS)-6-benzyloxy-2,3,4,4a,5,6,7,7a-octahydrocyclopenta[b][1,4]oxazine

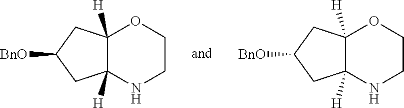 [SMILES: c1ccc(CO[C@H]2C[C@@H]3OCCN[C@@H]3C2)cc1 ; c1ccc(CO[C@H]2C[C@@H]3NCCO[C@@H]3C2)cc1]

To a solution of rac-(4aR,6R,7aS)-6-benzyloxy-4-(4-nitrophenyl)sulfonyl-3,4a,5,6,7,7a-hexahydro-2H-cyclopenta[b][1,4]oxazine (1.20 g, 2.76 mmol) in 12 ml of DMF was added 2-sulfanylacetic acid (611 mg, 6.63 mmol) and LiOH·H₂O (649 mg, 15.5 mmol). The solution was stirred at rt overnight. The reaction mixture was quenched with water and extracted with DCM (3 times). The combined organic layers were washed by saturated LiCl, dried over anhydrous Na₂SO₄, filtered and concentrated under reduced pressure. The residue was purified by flash chromatography on silica gel eluting with DCM/(7N NH₃/MeOH) (100/0 to 0/100) to give the title compound as a solid (190 mg, 28.3% yield).

LCMS (Method Z4): Rt=1.33; [M+H]⁺=234.0

### Int 466.15: rac-(4aR,6R,7aS)-6-benzyloxy-4-[6-(2-benzyloxy-4,6-dimethyl-phenyl)pyridazin-3-yl]-3,4a,5,6,7,7a-hexahydro-2H-cyclopenta[b][1,4]oxazine

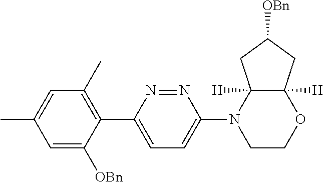 [SMILES: Cc1cc(C)c(-c2ccc(N3CCO[C@@H]4C[C@@H](OCc5ccccc5)C[C@@H]43)nn2)c(OCc2ccccc2)c1]

Under Ar, to a solution of 3-(2-benzyloxy-4,6-dimethyl-phenyl)-6-chloro-pyridazine (Int 466.4, 265.0 mg, 0.814 mmol) and rac-(4aR,6R,7aS)-6-benzyloxy-2,3,4,4a,5,6,7,7a-octahydrocyclopenta[b][1,4]oxazine (Int 466.14-190 mg, 0.814 mmol) in 5 ml of anhydrous toluene were added Cs₂CO₃ (796 mg, 2.44 mmol), Pd₂(dba)₃ (47 mg, 0.081 mmol) and [1-(2-diphenylphosphanyl-1-naphthyl)-2-naphthyl]-diphenyl-phosphane (101 mg, 0.163 mmol). The solution was purged again with Ar and stirred at 110° C. overnight under argon. After cooling down to rt, the mixture was poured into water and extracted with ethyl acetate (3 times). The combined organic layers were washed by brine, dried over anhydrous Na₂SO₄, filtered and concentrated under reduced pressure. The residue was purified by flash chromatography on silica gel eluting with petroleum ether/EA (100/0 to 0/100) to give the title compound as a solid (95 mg, 22.4% yield).

LCMS (Method Z4): Rt=2.42; MS m/z [M+H]⁺=522.0

### Int 466.16: 3,5-dimethyl-2-[6-[rac-(4aR,6R,7aS)-6-benzyloxy-3,4a,5,6,7,7a-hexahydro-2H-cyclopenta[b][1,4]oxazin-4-yl]pyridazin-3-yl]phenol

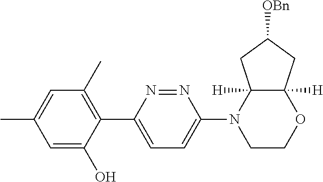 [SMILES: Cc1cc(C)c(-c2ccc(N3CCO[C@@H]4C[C@@H](OCc5ccccc5)C[C@@H]43)nn2)c(O)c1]

To a solution of rac-(4aR,6R,7aS)-6-benzyloxy-4-[6-(2-benzyloxy-4,6-dimethyl-phenyl)pyridazin-3-yl]-3,4a,5,6,7,7a-hexahydro-2H-cyclopenta[b][1,4]oxazine (95 mg, 0.128 mmol) in 5 ml of THF was added Pd(OH)₂ (10% wet, 50 mg). The mixture was purged with hydrogen and stirred at 40° C. overnight under hydrogen. The resulting mixture was filtered. The filtrate was concentrated under reduced pressure. The residue was purified by flash chromatography on silica gel eluting with petroleum ether/EA (100/0 to 0/100) to give the title compound as a solid (40 mg, yield 50.8%).

LCMS (Method Z4): Rt=2.11; MS m/z [M+H]⁺=432.0

### Int 466.17: rac-(4aR,6R,7aS)-4-[6-(2-hydroxy-4,6-dimethyl-phenyl)pyridazin-3-yl]-3,4a,5,6,7,7a-hexahydro-2H-cyclopenta[b][1,4]oxazin-6-ol

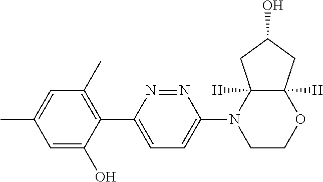 [SMILES: Cc1cc(C)c(-c2ccc(N3CCO[C@@H]4C[C@@H](O)C[C@@H]43)nn2)c(O)c1]

To the solution of 3,5-dimethyl-2-[6-[rac-(4aR,6R,7aS)-6-benzyloxy-3,4a,5,6,7,7a-hexahydro-2H-cyclopenta[b][1,4]oxazin-4-yl]pyridazin-3-yl]phenol (40 mg, 0.093 mmol) in 5 ml of EtOH was added Pd—C (10% wet, 20 mg) and Pd(OH)₂ (10% wet, 20 mg). The mixture was purged with hydrogen and stirred at 40° C. overnight under hydrogen. The resulting mixture was filtered. The filtrate was concentrated under reduced pressure. The residue was purified by flash chromatography on silica gel eluting with petroleum ether/EA (100/0 to 0/100) to give the title compound as a solid (18 mg, yield 56.9%).

LCMS (Method Z4): Rt=1.43; MS m/z [M+H]⁺=342.0

### (4aR,6R,7aS)-4-[6-(2-hydroxy-4,6-dimethyl-phenyl)pyridazin-3-yl]-3,4a,5,6,7,7a-hexahydro-2H-cyclopenta[b][1,4]oxazin-6-ol and (4aS,6S,7aR)-4-[6-(2-hydroxy-4,6-dimethyl-phenyl)pyridazin-3-yl]-3,4a,5,6,7,7a-hexahydro-2H-cyclopenta[b][1,4]oxazin-6-ol (Examples 466 and 467)

Both enantiomers of rac-(4aR,6R,7aS)-4-[6-(2-hydroxy-4,6-dimethyl-phenyl)pyridazin-3-yl]-3,4a,5,6,7,7a-hexahydro-2H-cyclopenta[b][1,4]oxazin-6-ol (18 mg) were separated by SFC (Column: IC 25*250 mm, 10 um (Daicel), Column temperature: 35° C., Mobile phase: CO₂/MEOH[0.2% NH₃ (7M in MeOH)]=60/40, Flow rate: 120 ml/min) to provide (4aR,6R,7aS)-4-[6-(2-hydroxy-4,6-dimethyl-phenyl)pyridazin-3-yl]-3,4a,5,6,7,7a-hexahydro-2H-cyclopenta[b][1,4]oxazin-6-ol (first enantiomer eluting at Rt=5.51 (6.10 mg, Example 466) as white solid and (4aS,6S,7aR)-4-[6-(2-hydroxy-4,6-dimethyl-phenyl)pyridazin-3-yl]-3,4a,5,6,7,7a-hexahydro-2H-cyclopenta[b][1,4]oxazin-6-ol (second enantiomer eluting at Rt=7.05 (6.70 mg, Example 467) as white solid.

The absolute configurations cannot be assigned at this stage.

### Synthesis of Intermediates

### 5-methoxy-3-methyl-2-(4,4,5,5-tetramethyl-1,3,2-dioxaborolan-2-yl)phenol

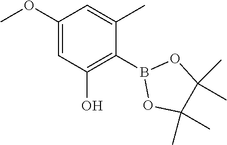

### Int 96.1: 2-bromo-5-methoxy-3-methyl-phenol

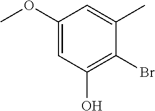

To a solution of 3-methoxy-5-methyl-phenol (43.4 mmol, 6 g) in 60 ml of anhydrous DCM pre-cooled to 10° C. was added N-bromosuccinimide (47.8 mmol, 8.5 g) by portions while maintaining temperature at 15° C. The mixture was stirred at 15° C. for 10 min then diluted with 30 ml of petroleum ether and filtered off. The cake was rinsed with 10 ml of a 30/70 mixture of DCM/petroleum ether and the filtrate concentrated under reduced pressure. The residue was purified by flash chromatography on silica gel eluting with a gradient of 0 to 100% of diisopropylether in petroleum ether to give the title compound as a white solid (5.2 g, 55% yield).

LCMS (Method H): Rt=1.60 min; MS m/z [M−H]⁺ 215

### Int 96.2: 5-methoxy-3-methyl-2-(4,4,5,5-tetramethyl-1,3,2-dioxaborolan-2-yl)phenol

Under Ar at 0° C., to a solution of 2-bromo-5-methoxy-3-methyl-phenol (4.6 mmol, 1 g) in 20 ml of anhydrous 1,4-dioxane was added Et₃N (10.6 mmol, 1.48 ml) and 4,4,5,5-tetramethyl-1,3,2-dioxaborolane (12.9 mmol, 1.83 ml). Then, were added diacetoxypalladium (0.23 mmol, 52 mg) and dicyclohexyl-(2-phenylphenyl)phosphane (0.46 mmol, 161 mg) and the mixture was stirred at 75° C. for 1.5 h. After cooling down to rt, the mixture was diluted with diethylether and washed with water. The organic layer was dried over Na₂SO₄, filtered and concentrated under reduced pressure. The residue was purified by flash chromatography on silica gel eluting with a gradient of 0 to 50% of diethylether in petroleum ether to give the title compound as a yellow solid (880 mg, 72% yield).

LCMS (Method L): Rt=1.01 min; MS m/z [M+H]⁺ 265

### (2,4-dichloro-6-hydroxy-phenyl)boronic acid

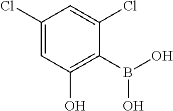 [SMILES: OB(O)c1c(O)cc(Cl)cc1Cl]

### Int 58.1: 3,5-dichloro-2-iodo-phenol

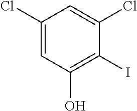

To a cooled (0-5° C.) suspension of NaH (60% in oil—371 mmol, 14.8 g) in 140 ml of toluene, was slowly added a solution of 3,5-dichlorophenol (177 mmol, 30 g) in 150 ml of toluene while keeping temperature below 10° C. The mixture was stirred for 45 min at 0-5° C., then a solution of iodine (177 mmol, 44.8 g) in 330 ml of toluene was slowly added while keeping temperature below 10° C. The mixture was stirred at 0-5° C. for 30 min, then quenched with 135 ml of an aqueous 2N HCl solution. The organic layer was successively washed with aqueous 10% sodium bisulfite solution and water then dried over Na₂SO₄, filtered and concentrated under reduced pressure to give the title compound as a white solid (33 g, 66% yield).

LCMS (Method I): Rt=1.91 min; MS m/z [M−H]⁺ 287

### Int 58.2: 1,5-dichloro-2-iodo-3-[(4-methoxyphenyl)methoxy]benzene

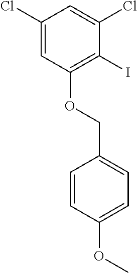 [SMILES: COc1ccc(COc2cc(Cl)cc(Cl)c2I)cc1]

A suspension of 3,5-dichloro-2-iodo-phenol (94.2 mmol, 27.2 g), 1-(chloromethyl)-4-methoxy-benzene (98.9 mmol, 15.5 g) and K₂CO₃ (113 mmol, 15.6 g) in 100 ml of DMF was stirred at 80° C. for 1 h. After cooling down to rt and diluted with 800 ml of diethyether and 300 ml of water, the organic layer was washed with water (3 times), dried over Na₂SO₄, filtered and concentrated under reduced pressure. The residue was triturated in a mixture of pentane and diethylether (80/20), filtered off and the solid washed with pentane and dried under reduced pressure to give the title compound as a white solid (30 g, 78% yield) LCMS (Method I): Rt=2.54 min; MS m/z [M+H]⁺ 409

### Int 58.3: 2-[2,4-dichloro-6-[(4-methoxyphenyl)methoxy]phenyl]-4,4,5,5-tetramethyl-1,3,2-dioxaborolane

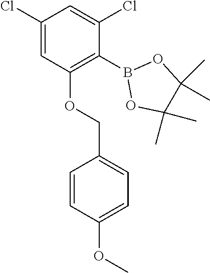 [SMILES: COc1ccc(COc2cc(Cl)cc(Cl)c2B2OC(C)(C)C(C)(C)O2)cc1]

Under Ar, at 0° C., to a solution of 1,5-dichloro-2-iodo-3-[(4-methoxyphenyl)methoxy]benzene (36.7 mmol, 15 g) in 160 ml of anhydrous 1,4-dioxane was added Et₃N (73.3 mmol, 10 ml), and slowly 4,4,5,5-tetramethyl-1,3,2-dioxaborolane (73.3 mmol, 10 ml). Then, was added diacetoxypalladium (1.83 mmol, 412 mg) and dicyclohexyl-(2-phenylphenyl)phosphane (3.67 mmol, 1.29 g) and the mixture was stirred at 80° C. for 1 h. After cooling down, the mixture was diluted with diethylether and pentane (80/20) and washed twice with water. The organic layer was dried over Na₂SO₄, filtered and concentrated under reduced pressure. The residue was purified by flash chromatography on silica gel eluting with a gradient of 0 to 50% of diethylether in cyclohexane to give the title compound as colorless oil (9.27 g, 62% yield).

LCMS (Method N): Rt=2.35 min; MS m/z [M+Na]⁺ 431

### Int 58.4: (2,4-dichloro-6-hydroxy-phenyl)boronic acid

To a cooled (−10° C.) solution of 2-[2,4-dichloro-6-[(4-methoxyphenyl)methoxy]phenyl]-4,4,5,5-tetramethyl-1,3,2-dioxaborolane (22.7 mmol, 9.2 g) and anisole (92 mmol, 10 ml) in 100 ml of DCM was added dropwise TFA (453 mmol, 34 ml) for 20 min. The mixture was stirred at 0° C., then quenched with 300 ml of an aqueous saturated NH₄Cl solution. 200 ml of water was added. The aqueous layer was extracted twice with DCM and the combined organic layers were dried over Na₂SO₄, filtered off and concentrated under reduced pressure. The residue was used without further purification for next step.

LCMS (Method I): Rt=1.30 min; MS m/z [M−H]⁺ 205

### 2-(4-chloro-2-methoxy-6-methyl-phenyl)-4,4,5,5-tetramethyl-1,3,2-dioxaborolane

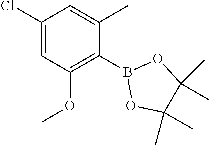 [SMILES: COc1cc(Cl)cc(C)c1B1OC(C)(C)C(C)(C)O1]

### Int 63.1: 4-chloro-2-methoxy-6-methyl-aniline

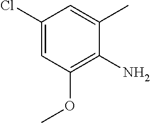

To a solution of 2-bromo-4-chloro-6-methyl-aniline (104 mmol, 23 g) in 15 ml of MeOH was added a 5.4 N sodium methoxide solution in MeOH (522 mmol, 97 ml), then Copper(I) iodide (120 mmol, 22.8 g) and the mixture was stirred at reflux for 2 h. After cooling to rt, 1 L of water was added and the resulting solution was extracted with EtOAc. The mixture was filtered over Dicalite® and the aqueous layer was extracted with EtOAc. The combined organic layers were dried over Na₂SO₄, filtered and concentrated under reduced pressure. The residue was purified by flash chromatography on silica gel eluting with a gradient of 0 to 10% of EtOAc in heptane to give the title compound as a brown oil (12.6 g, 70% yield).

LCMS (Method H): Rt=1.53 min; MS m/z [M+H]⁺ 172

### Int 63.2: 5-chloro-2-iodo-1-methoxy-3-methyl-benzene

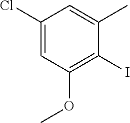

At 0° C., to a solution of 4-chloro-2-methoxy-6-methyl-aniline (11.65 mmol, 2 g) in 40 ml of ACN and 45 ml of 4 N aqueous HCl solution was added sodium iodide (23.3 mmol, 3.5 g).

This solution was cooled to −5° C., then was added dropwise an aqueous sodium nitrite solution (12.23 mmol, 844 mg in 4 ml of water) and the mixture was stirred 1 h at −5° C. After warm-up to rt, EtOAc and 10% aqueous sodium bisulfite solution were added. The organic layer was washed with water, brine, dried over Na₂SO₄, filtered and concentrated under reduced pressure. The residue was purified by flash chromatography on silica gel eluting with a gradient of 0 to 5% of EtOAc in heptane to give the title compound as a white solid (1.08 g, 33% yield).

LCMS (Method H): Rt=2.38 min; No ionization

### Int 63.3: 2-(4-chloro-2-methoxy-6-methyl-phenyl)-4,4,5,5-tetramethyl-1,3,2-dioxaborolane

Under Ar, at 0° C., to a solution of 5-chloro-2-iodo-1-methoxy-3-methyl-benzene (3.54 mmol, 1 g) in 17 ml of anhydrous 1,4-dioxane was added Et₃N (7.1 mmol, 1 ml) and 4,4,5,5-tetramethyl-1,3,2-dioxaborolane (7.08 mmol, 1 ml). Then, was added diacetoxypalladium (0.18 mmol, 40 mg) and dicyclohexyl-(2-phenylphenyl)phosphane (0.354 mmol, 124 mg) and the mixture was stirred at 80° C. for 1.5 h. After cooling down, the mixture was diluted with diethylether and pentane, and washed with water. The organic layer was dried over Na₂SO₄, filtered and concentrated under reduced pressure. The residue was purified by flash chromatography on silica gel eluting with a gradient of 0 to 70% of diethylether in pentane to give the title compound as a yellow solid (880 mg, 88% yield).

LCMS (Method I): Rt=2.27 min; MS m/z [M+H]⁺ 283

### 3-methyl-2-(4,4,5,5-tetramethyl-1,3,2-dioxaborolan-2-yl)-5-(trifluoromethyl)phenol

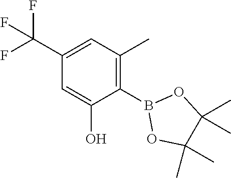 [SMILES: Cc1cc(C(F)(F)F)cc(O)c1B1OC(C)(C)C(C)(C)O1]

### Step 1: 3-methyl-5-(trifluoromethyl)phenol

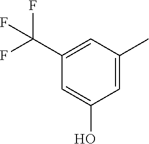

To a solution of 1-bromo-3-methyl-5-(trifluoromethyl)benzene (41.8 mmol, 10 g) in 100 ml of 1,4-dioxane and 10 ml of water was added LiOH·H₂O (125.5 mmol, 5.27 g), Pd₂(dba)₃ (837 μmol, 766 mg) and ditert-butyl-[2-(1,3,5-triphenylpyrazol-4-yl)pyrazol-3-yl]phosphane (1.67 mmol, 848 mg). The mixture was degassed and purged with N₂, and then stirred at 100° C. for 16 h. After cooling to rt, the solution was filtered and washed with ethyl acetate. The filtrate was washed with 1 M HCl, dried over Na₂SO₄, filtered and concentrated under reduced pressure. The residue was purified by flash chromatography on silica gel eluting with a gradient of 0 to 70% of Ethyl acetate in Petroleum ether to give the title compound as a yellow oil (5.3 g, 72% yield).

1HNMR (400 MHz, CDCl₃) δ=7.02 (s, 1H), 6.89 (s, 1H), 6.83 (s, 1H), 5.05 (d, J=2.8 Hz, 1H), 2.37 (s, 3H).

### Step 2: 2-iodo-3-methyl-5-(trifluoromethyl)phenol

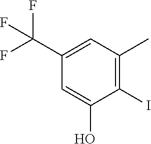

At 0° C., under N₂, to a solution of 3-methyl-5-(trifluoromethyl)phenol (22.7 mmol, 4 g) in 80 ml of toluene was added NaH (45.4 mmol, 1.8 g, 60% purity) and the solution was stirred at 0° C. for 0.5 h. Then, 12 (22.7 mmol, 5.8 g) was slowly added, and the mixture was stirred at 0° C. for 3 h. The reaction was quenched by addition of 100 ml of water at 0° C., then adjusted pH to 5 with 2 M HCl. The solution was extracted with ethyl acetate (3 times). The combined organic layers were washed with brine, dried over Na₂SO₄, filtered and concentrated under reduced pressure. The residue was purified by flash chromatography on silica gel eluting with a gradient of 0 to 3% of Ethyl acetate in Petroleum ether to give the title compound as a yellow oil (3.8 g, 55% yield).

1H NMR (400 MHz, CDCl₃) δ=7.07 (s, 2H), 5.66 (s, 1H), 2.52 (s, 3H).

### Step 3: 2-iodo-1-(methoxymethoxy)-3-methyl-5-(trifluoromethyl)benzene

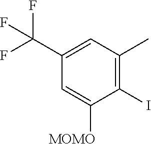

At 0° C., under N₂, to a solution of 2-iodo-3-methyl-5-(trifluoromethyl)phenol (12.6 mmol, 3.8 g) in 80 ml of THF was added NaH (18.9 mmol, 755 mg, 60% purity) and the mixture was stirred at 0° C. for 0.5 h. Then, bromo(methoxy)methane (15.1 mmol, 1.9 g) was dropwise added at 0° C. and the mixture was stirred at 25° C. for 1 h. The reaction was quenched by addition of 50 ml of saturated NH₄Cl at 0° C., then diluted with H₂O and extracted with ethyl acetate (3 times). The combined organic layers were washed with brine, dried over Na₂SO₄, filtered and concentrated under reduced pressure. The residue was purified by flash chromatography on silica gel eluting with a gradient of 0 to 1% of Ethyl acetate in Petroleum ether to give the title compound as a colorless oil (4 g, 91% yield).

1H NMR (400 MHz, CDCl₃) δ=7.18 (s, 1H), 7.10 (s, 1H), 5.29 (s, 2H), 3.53 (s, 3H), 2.54 (s, 3H).

### Step 4: 2-(2-(methoxymethoxy)-6-methyl-4-(trifluoromethyl)phenyl)-4,4,5,5-tetramethyl-1,3,2-dioxaborolane

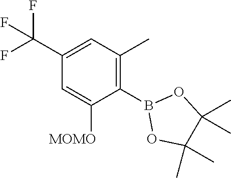

Under N₂, to a solution of 2-iodo-1-(methoxymethoxy)-3-methyl-5-(trifluoromethyl)benzene (11.7 mmol, 4.0 g) in 55 ml of 1,4-dioxane was added Et₃N (86.6 mmol, 12 ml), biphenyl-2-yl-dicyclohexylphosphane (2.81 mmol, 984 mg) and Pd(OAc)₂ (1.4 mmol, 315 mg). The solution was degassed and purged with N₂, then 4,4,5,5-tetramethyl-1,3,2-dioxaborolane (58.5 mmol, 7.5 g) was added and the mixture was stirred at 80° C. for 12 h. After cooling down to rt, the reaction was filtered. The filtrate was washed with saturated NH₄Cl, water and brine. The organic layer was dried over Na₂SO₄, filtered and concentrated under reduced pressure. The residue was purified by flash chromatography on silica gel eluting with a gradient of 0 to 5% of Ethyl acetate in Petroleum ether to give the title compound as a yellow solid (2 g, 49% yield).

1H NMR (400 MHz, CDCl₃) δ=7.06 (s, 2H), 5.17 (s, 2H), 3.48 (s, 3H), 2.40 (s, 3H), 1.40 (s, 12H).

### Step 5: 3-methyl-2-(4,4,5,5-tetramethyl-1,3,2-dioxaborolan-2-yl)-5-(trifluoromethyl) phenol

At 0° C., to a solution of 2-(2-(methoxymethoxy)-6-methyl-4-(trifluoromethyl)phenyl)-4,4,5,5-tetramethyl-1,3,2-dioxaborolane (1.44 mmol, 500 mg) in 5 ml of EtOAc was added 2.5 ml of a 4N solution of HCl in EtOAc and the mixture was stirred at 0° C. for 1 h. Then, the reaction mixture was concentrated under reduced pressure to give 3-methyl-2-(4,4,5,5-tetramethyl-1,3,2-dioxaborolan-2-yl)-5-(trifluoromethyl)phenol (446 mg, crude) as a yellow oil, which was used directly for the next step.

1H NMR (400 MHz, CDCl₃) δ=8.71 (s, 1H), 6.94 (d, J=11.2 Hz, 2H), 2.54 (s, 3H), 1.39 (s, 12H).

### N-(7-azaspiro[3.5]nonan-2-yl)acetamide;2,2,2-trifluoroacetic acid

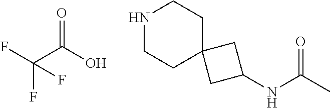

### Int 47.1: tert-butyl 2-acetamido-7-azaspiro[3.5]nonane-7-carboxylate

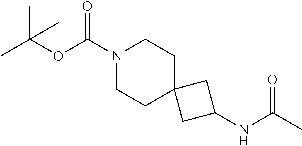 [SMILES: CC(=O)NC1CC2(CCN(C(=O)OC(C)(C)C)CC2)C1]

To a solution of tert-butyl 2-amino-7-azaspiro[3.5]nonane-7-carboxylate (2.08 mmol, 500 mg) in 5 ml of DCM were successively added Et₃N (2.08 mmol, 0.28 ml), N,N-dimethylpyridin-4-amine (0.104 mmol, 13 mg) and acetic anhydride (3.12 mmol, 0.3 ml) and the mixture was stirred at rt for 18 h. The solution was diluted with DCM and saturated aqueous solution of NaHCO₃. The organic layer was dried over Na₂SO₄, filtered and concentrated under reduced pressure. The residue was purified by flash chromatography on silica gel eluting with a gradient of 0 to 5% of MeOH in DCM to give the title compound as a yellow foam (600 mg, 100% yield).

LCMS (Method O): Rt=1.22 min; MS m/z [M+H]⁺ 283

### Int 47.2: N-(7-azaspiro[3.5]nonan-2-yl)acetamide;2,2,2-trifluoroacetic acid

A solution of tert-butyl 2-acetamido-7-azaspiro[3.5]nonane-7-carboxylate (1.06 mmol, 300 mg) in 18 ml of DCM and 2 ml of TFA was stirred at rt for 2 h. The solution was concentrated under reduced pressure to give the title compound as a colourless oil (430 mg, 98% yield).

LCMS (Method O): Rt=0.24 min; MS m/z [M+H]⁺ 183

### N-[[(3R)-3-piperidyl]methyl]acetamide;2,2,2-trifluoroacetic acid

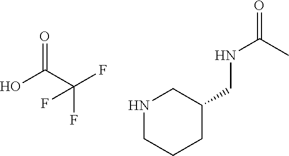

### Int 58.6: tert-butyl (3S)-3-(acetamidomethyl)piperidine-1-carboxylate

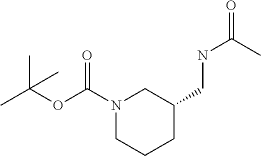 [SMILES: CC(=O)NC[C@@H]1CCCN(C(=O)OC(C)(C)C)C1]

To a solution of tert-butyl (3S)-3-(aminomethyl)piperidine-1-carboxylate (4.66 mmol, 1 g) in 20 ml of DCM was successively added Et₃N (4.66 mmol, 0.64 ml), DMAP (0.233 mmol, 28.5 mg) and acetic anhydride (7 mmol, 0.66 ml) and the mixture was stirred at rt for 18 h. then to the solution was added DCM and saturated aq. solution of NaHCO₃. The organic layer was dried over Na₂SO₄, filtered and concentrated under reduced pressure. The residue was purified by flash chromatography on silica gel eluting with a gradient of 0 to 5% of MeOH in DCM to give the title compound as a yellow oil (800 mg, 67% yield).

LCMS (Method O): Rt=1.15 min; MS m/z [M+H]⁺ 257

### Int 58.7: N-[[(3R)-3-piperidyl]methyl]acetamide;2,2,2-trifluoroacetic acid

A solution of tert-butyl (3S)-3-(acetamidomethyl)piperidine-1-carboxylate (1.56 mmol, 400 mg) in 18 ml of DCM and 2 ml of TFA was stirred at rt for 2 h. The solution was then concentrated under reduced pressure to give the title compound as a colourless oil (598 mg, 100% yield).

LCMS (Method O): Rt=0.28 min; MS m/z [M+H]⁺ 157

### benzyl rac-(4aR,8aS)-3-oxo-4,4a,5,7,8,8a-hexahydropyrido[4,3-b][1,4]oxazine-6-carboxylate

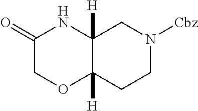 [SMILES: O=C1CO[C@H]2CCN(C(=O)OCc3ccccc3)C[C@H]2N1]

### Int 10.2: 4H-pyrido[4,3-b][1,4]oxazin-3-one

To a solution of 3-aminopyridin-4-ol (227 mmol, 25 g) in 400 ml of ACN was added dropwise 2-chloroacetyl chloride (250 mmol, 19.86 ml), and the solution was stirred at 25° C. for 0.5 h. Then K₂CO₃ (568 mmol, 78.5 g) was added, and the mixture was stirred at 85° C. for 10 h. After cooling to rt, the reaction mixture was concentrated under reduced pressure and 100 ml of a solution of EtOAc/MeOH (1/1) was added. The mixture was filtered and concentrated under reduced pressure to give a residue, which was purified by flash chromatography on silica gel using DCM/MeOH (from 10/1 to 1/1) to give the title compound as a yellow solid (7 g, 20% yield).

¹H NMR (400 MHz, CDCl₃) δ=8.21 (d, J=5.6 Hz, 1H), 8.14 (s, 1H), 6.93 (d, J=5.6 Hz, 1H), 4.74 (s, 2H).

### Int 10.3: 6-benzyl-4H-pyrido[4,3-b][1,4]oxazin-6-ium-3-one; bromide

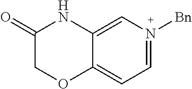

To a solution of 4H-pyrido[4,3-b][1,4]oxazin-3-one (46.6 mmol, 7 g) in 100 ml of DCM and 20 ml of MeOH was added benzylbromide (230 mmol, 162 ml), and the mixture was stirred at 25° C. for 8 h. The reaction mixture was filtered and the filter cake was dried under vacuum to give 10 g of crude 6-benzyl-3-oxo-3,4-dihydro-2H-pyrido[4,3-b][1,4]oxazin-6-ium bromide as a white solid, which was used without further purification for next step.

¹H NMR (400 MHz, DMSO-d₆) δ=8.76 (br d, J=6.8 Hz, 1H), 8.36 (s, 1H), 7.64 (d, J=6.8 Hz, 1H), 7.53-7.33 (m, 6H), 5.75 (s, 2H), 5.00 (s, 2H)

### Int 10.4: 6-benzyl-4,5,7,8-tetrahydropyrido[4,3-b][1,4]oxazin-3-one

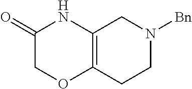 [SMILES: O=C1COC2=C(CN(Cc3ccccc3)CC2)N1]

At 0° C., to a solution of 6-benzyl-4H-pyrido[4,3-b][1,4]oxazin-6-ium-3-one; bromide (33.2 mmol, 8 g) in 120 ml of MeOH was added NaBH₄ (232 mmol, 8.78 g) by batches over 2 h and the mixture was then stirred at 0° C. for 1 h. The reaction solution was diluted with 50 ml of water and concentrated under reduced pressure. The aqueous layer was extracted with EtOAc (3 times). The combined organic layers were dried over Na₂SO₄, filtered and concentrated under reduced pressure. The residue was purified by flash chromatography on silica gel using Petroleum ether/Ethyl acetate (from 5/1 to 1/1) to give the title compound as a white solid (4.1 g, 50% yield).

¹H NMR (400 MHz, CDCl₃) δ=8.07 (br s, 1H), 7.37-7.27 (m, 5H), 4.41 (s, 2H), 3.64 (s, 2H), 3.03 (t, J=2.0 Hz, 2H), 2.66 (t, J=5.6 Hz, 2H), 2.29-2.17 (m, 2H).

### Int 10.5: rac-(4aR,8aS)-4a,5,6,7,8,8a-hexahydro-4H-pyrido[4,3-b][1,4]oxazin-3-one

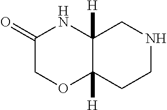 [SMILES: O=C1CO[C@H]2CCNC[C@H]2N1]

Under N₂ atmosphere, to a solution of 6-benzyl-4,5,7,8-tetrahydropyrido[4,3-b][1,4]oxazin-3-one (21 mmol, 5.1 g) in 50 ml of MeOH was added Pd/C (2.2 g). The suspension was degassed and purged with H₂ for 3 times. The mixture was stirred under H₂ (15 Psi) at 25° C. for 8 h. The reaction mixture was then filtered and the filtrate was concentrated under reduced pressure to give 3.5 g of crude (4aR, 8aS)-hexahydro-2H-pyrido[4,3-b][1,4]oxazin-3(4H)-one as a white solid, which was used without further purification for next step.

¹H NMR (400 MHz, CDCl₃) δ=4.35-4.26 (m, 1H), 4.24-4.15 (m, 1H), 3.97 (br d, J=2.4 Hz, 1H), 3.41-3.26 (m, 1H), 3.05-2.73 (m, 4H), 1.97 (br dd, J=2.4, 14.4 Hz, 1H), 1.87-1.73 (m, 2H).

### Int 10.6: benzyl rac-(4aR,8aS)-3-oxo-4,4a,5,7,8,8a-hexahydropyrido[4,3-b][1,4]oxazine-6-carboxylate

At 0° C., to a solution of rac-(4aR,8aS)-4a,5,6,7,8,8a-hexahydro-4H-pyrido[4,3-b][1,4]oxazin-3-one (22.4 mmol, 3.5 g) in 30 ml of DCM was added Et₃N (45 mmol, 6.52 ml) and CbzCl (45 mmol, 7.65 g). The solution was then stirred at 25° C. for 8 h. The solution was concentrated under reduced pressure to give a residue which was purified by flash chromatography on silica gel using Petroleum ether/Ethyl acetate (from 3/1 to 1/1) to give the title compound as a colorless oil (6.5 g, 57% yield).

¹H NMR (400 MHz, CDCl₃) δ=7.43-7.30 (m, 5H), 6.46 (br s, 1H), 5.13 (br s, 2H), 4.37-4.18 (m, 2H), 3.97 (br d, J=2.8 Hz, 2H), 3.36 (br s, 1H), 3.09 (br s, 1H), 2.02-1.74 (m, 2H).

### benzyl rac-(4aS,8aR)-1,2,3,4a,5,7,8,8a-octahydropyrido[3,4-b][1,4]oxazine-6-carboxylate

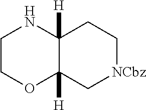 [SMILES: O=C(OCc1ccccc1)N1CC[C@H]2NCCO[C@H]2C1]

### Int 4.1: benzyl 7-oxa-3-azabicyclo[4.1.0]heptane-3-carboxylate

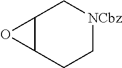 [SMILES: O=C(OCc1ccccc1)N1CCC2OC2C1]

At 0° C., to a solution of benzyl 3,6-dihydro-2H-pyridine-1-carboxylate (110.5 mmol, 24 g) in 60 ml of DCM was added, m-CPBA (133 mmol, 27 g). The reaction was stirred for 1 h at 0° C. and 7 h at 25° C. Then, the reaction was diluted with DCM, washed with saturated aqueous solution of Na₂S₂O₃, sat. aq. solution of NaHCO₃ and brine. The organic layer was dried over Na₂SO₄, filtered and concentrated under reduced pressure. The residue was purified by flash chromatography on silica gel using DCM/MeOH (from 10/1 to 2/1) to give the title compound as a yellow oil (22 g, 86% yield).

¹H NMR (400 MHz, CDCl₃) δ=7.43-7.28 (m, 5H), 5.13 (s, 2H), 4.05-3.88 (m, 1H), 3.85-3.71 (m, 1H), 3.53 (br s, 1H), 3.36-3.14 (m, 3H), 2.17-2.01 (m, 1H), 1.95 (br s, 1H).

### Int 4.2: benzyl rac-(3S,4S)-4-(tert-butoxycarbonylamino)-3-hydroxy-piperidine-1-carboxylate

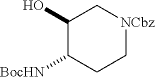 [SMILES: CC(C)(C)OC(=O)N[C@H]1CCN(C(=O)OCc2ccccc2)C[C@@H]1O]

A solution of benzyl 7-oxa-3-azabicyclo[4.1.0]heptane-3-carboxylate (94.7 mmol, 22 g) in 20 ml of EtOH and 20 ml of saturated ammonium hydroxide aqueous solution was stirred at 70° C. for 5 h. After cooling to rt, EtOAc and water were added to the solution. The organic layer was dried over Na₂SO₄, filtered and concentrated under reduced pressure. To this concentrate was added a solution of tert-butoxycarbonyl tert-butyl carbonate (104 mmol, 22.75 g) and Et₃N (94.8 mmol, 9.6 g) in 200 ml of DCM and the resulting solution was stirred for 2 h. Then, 100 ml of water was added, and the solution was extracted with EtOAc (3 times). The combined organic layers were washed with brine, dried over Na₂SO₄, filtered and concentrated under reduced pressure. The residue was purified by flash chromatography on silica gel using Petroleum ether/Ethyl acetate (from 10/1 to 2/1) to give the title compound as a white solid (11 g, 33% yield).

¹H NMR (400 MHz, CDCl₃) δ=7.33-7.21 (m, 5H), 5.05 (s, 2H), 4.55 (br s, 1H), 4.26 (br s, 1H), 3.46-3.25 (m, 2H), 2.87-2.67 (m, 1H), 2.60 (br dd, J=10.8, 12.4 Hz, 1H), 1.91-1.80 (m, 1H), 1.38 (s, 9H).

### Int 4.3: benzyl rac-(3R,4S)-3-benzoyloxy-4-(tert-butoxycarbonylamino)piperidine-1-carboxylate

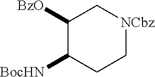

A mixture of DIAD (30.8 mmol, 6.2 g), PPh₃ (30.8 mmol, 8.1 g) in 40 ml of THF was degassed and purged with N₂ for 3 times, then stirred at 0° C. for 0.5 hr. A solution of benzyl rac-(3S,4S)-4-(tert-butoxycarbonylamino)-3-hydroxy-piperidine-1-carboxylate (25.7 mmol, 9 g) and benzoic acid (30.8 mmol, 3.8 g) in 10 ml of THF was added, and the resulting mixture was stirred for 1.5 h under N₂ atmosphere. Water was added and the mixture was extracted with EtOAc (3 times). The combined organic layers were washed with brine, dried over Na₂SO₄, filtered and concentrated under reduced pressure. The residue was purified by flash chromatography on silica gel using Petroleum ether/Ethyl acetate (from 50/1 to 5/1) to give the title compound as a yellow oil (11 g, 94% yield).

¹H NMR (400 MHz, CDCl₃) δ=8.05-7.89 (m, 2H), 7.60 (q, J=7.2 Hz, 1H), 7.46 (br t, J=6.8 Hz, 2H), 7.39-7.27 (m, 2H), 7.24-7.07 (m, 2H), 7.00 (br d, J=7.2 Hz, 1H), 6.47 (br s, 3H), 5.35-5.02 (m, 2H), 4.77 (br d, J=8.0 Hz, 1H), 4.64-4.51 (m, 1H), 4.39 (br d, J=13.6 Hz, 1H), 3.99 (br d, J=3.6 Hz, 1H), 3.10 (br d, J=15.2 Hz, 1H), 2.92 (br t, J=10.8 Hz, 1H), 1.90 (br d, J=3.6 Hz, 2H), 1.49-1.35 (m, 9H)

### Int 4.4: benzyl rac-(3R,4S)-4-(tert-butoxycarbonylamino)-3-hydroxy-piperidine-1-carboxylate

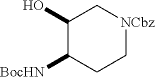 [SMILES: CC(C)(C)OC(=O)N[C@@H]1CCN(C(=O)OCc2ccccc2)C[C@@H]1O]

To a solution of benzyl rac-(3R,4S)-3-benzoyloxy-4-(tert-butoxycarbonylamino) piperidine-1-carboxylate (24.2 mmol, 11.0 g) in 30 ml of EtOH and 30 ml of H₂O was added K₂CO₃ (36.3 mmol, 5.0 g) and the mixture was stirred at 70° C. for 8 h. After cooling to rt, water was added, and the mixture was extracted with EtOAc (3 times). The combined organic layers were washed with brine, dried over Na₂SO₄, filtered and concentrated under reduced pressure. The residue was purified by flash chromatography on silica gel using Petroleum ether/Ethyl acetate (from 5/1 to 1/1) to give the title compound as a white solid (4.1 g, 48% yield).

¹H NMR (400 MHz, CDCl₃) δ=7.44-7.31 (m, 5H), 5.15 (s, 2H), 5.07-4.84 (m, 1H), 4.38-4.07 (m, 2H), 3.93 (br s, 1H), 3.79-3.63 (m, 1H), 3.14-3.00 (m, 1H), 2.98-2.86 (m, 1H), 1.74 (dt, J=4.4, 8.8 Hz, 2H), 1.46 (s, 9H).

### Int 4.5: benzyl rac-(3R,4S)-4-amino-3-hydroxy-piperidine-1-carboxylate

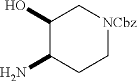 [SMILES: N[C@@H]1CCN(C(=O)OCc2ccccc2)C[C@@H]1O]

A mixture of benzyl rac-(3R,4S)-4-(tert-butoxycarbonylamino)-3-hydroxy-piperidine-1-carboxylate (11.7 mmol, 4.1 g) in 5 ml of 4M HCl/EtOAc was stirred at rt for 0.5 h. The reaction was concentrated under reduced pressure to give 3.45 g of crude benzyl (3S, 4R)-4-amino-3-hydroxypiperidine-1-carboxylate as a white solid, which was used without further purification for next step.

¹H NMR (400 MHz, DMSO-d₆) δ=8.04 (br s, 2H), 7.45-7.26 (m, 5H), 5.62 (br s, 1H), 5.07 (s, 2H), 4.07-3.79 (m, 3H), 3.26 (br d, J=3.2 Hz, 1H), 3.16-2.77 (m, 2H), 1.84-1.54 (m, 2H).

### Int 4.6: benzyl rac-(3R,4S)-3-hydroxy-4-[(4-nitrophenyl)sulfonylamino]piperidine-1-carboxylate

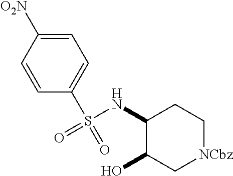 [SMILES: O=C(OCc1ccccc1)N1CC[C@H](NS(=O)(=O)c2ccc([N+](=O)[O-])cc2)[C@H](O)C1]

At 0° C., to a solution of 4-nitrobenzenesulfonyl chloride (14.9 mmol, 3.3 g) in 50 ml of DCM was added Et₃N (13.6 mmol, 1.4 g) and the mixture was stirred for 0.5 h. Then, benzyl rac-(3R,4S)-4-amino-3-hydroxy-piperidine-1-carboxylate (3.45 g, 13.78 mmol, 1.01 eq) was added at 0° C., and the reaction was stirred at rt for another 0.5 h. The reaction was diluted with water and extracted with DCM (3 times). The combined organic layers were washed with brine, dried over Na₂SO₄, filtered and concentrated under reduced pressure. The residue was purified by flash chromatography on silica gel using Petroleum ether/Ethyl acetate (from 50/1 to 10/1) to give the title compound as a white solid (2.6 g, 44% yield).

¹H NMR (400 MHz, DMSO-d₆) δ=8.39 (br d, J=8.8 Hz, 2H), 8.10 (br d, J=8.8 Hz, 2H), 7.43-7.21 (m, 5H), 5.02 (br s, 2H), 3.78 (br d, J=9.6 Hz, 2H), 3.52 (br s, 1H), 3.35 (br s, 1H), 3.08-2.95 (m, 1H), 2.91-2.74 (m, 1H), 1.75-1.54 (m, 1H), 1.29-1.16 (m, 1H).

### Int 4.7: benzyl rac-(4aS,8aR)-1-(4-nitrophenyl)sulfonyl-3,4a,5,7,8,8a-hexahydro-2H-pyrido[3,4-b][1,4]oxazine-6-carboxylate

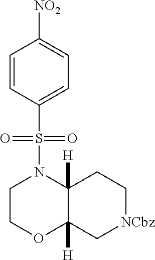 [SMILES: O=C(OCc1ccccc1)N1CC[C@@H]2[C@H](C1)OCCN2S(=O)(=O)c1ccc([N+](=O)[O-])cc1]

Under N₂ athmosthere, at 0° C., to a solution of 2-bromoethyl(diphenyl)sulfonium; trifluoromethanesulfonate (12.9 mmol, 5.7 g) in 30 ml of DCM was added NaH (26.3 mmol, 1.05 g, 60% purity). Benzyl rac-(3R,4S)-3-hydroxy-4-[(4-nitrophenyl)sulfonylamino]piperidine-1-carboxylate (4.8 mmol, 2.1 g) was added and the reaction was stirred at rt for 6 h. Then Et₃N (12.4 mmol, 1.7 ml) was added, and the solution was stirred for another 6 h. After that, water was added, and the solution was extracted with DCM (3 times). The combined organic layers were washed with brine, dried over Na₂SO₄, filtered and concentrated under reduced pressure. The residue was purified by flash chromatography on silica gel using Petroleum ether/EA (from 5/1 to 1/1) to give the title compound as a yellow solid (1.6 g, 72% yield).

¹H NMR (400 MHz, DMSO-d₆) δ=8.41 (d, J=8.8 Hz, 2H), 8.13 (d, J=8.8 Hz, 2H), 7.48-7.17 (m, 5H), 5.03 (br s, 2H), 4.09-3.92 (m, 3H), 3.88-3.79 (m, 1H), 3.52 (br d, J=11.2 Hz, 1H), 3.37 (br d, J=6.0 Hz, 1H), 3.31 (s, 1H), 3.26-3.00 (m, 2H), 2.95-2.74 (m, 1H), 1.93 (dq, J=4.8, 12.8 Hz, 1H), 1.33-1.22 (m, 1H).

### Int 4.8: 6-benzyl 1-tert-butyl rac-(4aS,8aR)-3,4a,5,7,8,8a-hexahydro-2H-pyrido[3,4-b][1,4]oxazine-1,6-dicarboxylate

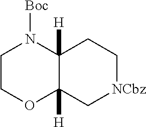 [SMILES: CC(C)(C)OC(=O)N1CCO[C@H]2CN(C(=O)OCc3ccccc3)CC[C@H]21]

To a solution of benzyl rac-(4aS,8aR)-1-(4-nitrophenyl)sulfonyl-3,4a,5,7,8,8a-hexahydro-2H-pyrido[3,4-b][1,4]oxazine-6-carboxylate (3.5 mmol, 1.6 g) in 15 ml DMF was added thioglycolic acid (9.0 mmol, 0.62 μl), followed by lithium hydroxide (17.4 mmol, 415 mg) and NH₃·H₂O (2.4 ml, 17.34 mmol, 28% purity) and the reaction was stirred for 6 h. EtOAc and water are added. The aqueous layer was extracted with EtOAc (3 times). The combined organic layers were dried over Na₂SO₄, filtered, and concentrated under reduced pressure. The residue was dissolved in 20 ml of DCM and then were added Et₃N (5.2 mmol, 724 μl), Boc₂O (5.20 mmol, 1.2 ml), and DMAP (520 μmol, 63 mg). After stirring for 2 h, the resulting mixture was poured into water and extracted with DCM (3 times). The combined organic layers were washed with brine, dried over Na₂SO₄, filtered and concentrated under reduced pressure. The residue was purified by flash chromatography on silica gel using Petroleum ether/Ethyl acetate (from 10/1 to 1/1) to give the title compound as a yellow oil (750 mg, 57% yield).

¹H NMR (400 MHz, CDCl₃) δ=7.43-7.30 (m, 5H), 5.15 (br d, J=17.6 Hz, 2H), 4.57-4.21 (m, 2H), 4.08-3.39 (m, 5H), 3.26-2.64 (m, 3H), 2.18-2.06 (m, 1H), 1.47 (s, 9H).

### Int 4.9: benzyl rac-(4aS,8aR)-1,2,3,4a,5,7,8,8a-octahydropyrido[3,4-b][1,4]oxazine-6-carboxylate

A mixture of 6-benzyl 1-tert-butyl rac-(4aS,8aR)-3,4a,5,7,8,8a-hexahydro-2H-pyrido[3,4-b][1,4]oxazine-1,6-dicarboxylate (1.7 mmol, 650 mg) in 5 ml of 4M HCl/EtOAc was stirred for 0.5 h. Then the reaction was concentrated under reduced pressure to give 460 mg of crude benzyl (4aS, 8aR)-octahydro-6H-pyrido [3,4-b][1,4] oxazine-6-carboxylate as a yellow oil, which was used without further purification for next step.

¹H NMR (400 MHz, DMSO-d₆) δ=7.49-7.23 (m, 5H), 5.05 (br s, 2H), 4.18-3.92 (m, 4H), 3.91-3.65 (m, 2H), 3.62-3.47 (m, 1H), 3.25-3.15 (m, 1H), 2.98 (br d, J=14.2 Hz, 2H), 2.18-2.04 (m, 1H), 1.70 (br dd, J=2.0, 9.2 Hz, 1H).

### 3-methyl-2-(4,4,5,5-tetramethyl-1,3,2-dioxaborolan-2-yl)-5-(trifluoromethoxy)phenol

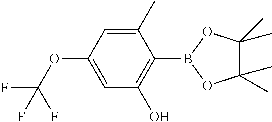 [SMILES: Cc1cc(OC(F)(F)F)cc(O)c1B1OC(C)(C)C(C)(C)O1]

### Step 1 Preparation of Int 121.1

1-bromo-3-methyl-5-(trifluoromethoxy)benzene (1 eq., 1.00 g, 0.00392 mol) and lithium hydroxide hydrate (3 eq., 56.0%, 0.881 g, 0.0118 mol) were dissolved in 1,4-dioxane (20.0 mL) and water (2.00 mL) and purged with N₂. Pd₂dba₃ (0.02 eq., 0.0451 g, 7.84e-5 mol) and BippyPhos (0.04 eq., 0.0795 g, 0.000157 mol) were added and purged another time. Then, 30 the mixture was stirred for 18 h at 100° C. The mixture was acidified with an aqueous solution of HCl 1M, diluted with EtOAc and washed with water. Combined organic layers were dried over MgSO₄, filtered, and evaporated in vacuo. The compound was purified by normal phase preparative LC (regular SiOH, 50 μm, 25 g interchim, dry loading (Celite®), mobile phase gradient: from heptane/EtOAc 100/0 to 85/15 over 10 CV. The fractions containing compound were combined and evaporated in vacuo to give Int 121.1 as a red oil (700 mg, 93%).

1H NMR (400 MHz, CDCl₃) δ (ppm): 6.64-6.50 (m, 3H), 5.01-4.92 (m, 1H), 2.31 (d, J=0.7 Hz, 3H).

LCMS (Method S): Rt=1.24 min; MS m/z [M+H]⁺=191.1

### Step 2 Preparation of Int 121.2

Int 121.1 (700 mg, 0.00364 mol, 1 eq) was dissolved in toluene (20.0 mL) at 0° C. Then, NaH (60% in mineral oil) (60.0%, 0.291 g, 0.00729 mol, 2 eq.) was added dropwise at the same temperature and stirred for 20 min. 12 (0.925 g, 0.00364 mol, 1 eq.) was added at 0° C. and the mixture was stirred for 1 h at 0° C.

The mixture was acidified with an aqueous solution of HCl 1M, diluted with EtOAc and washed with water. Combined organic layers were dried over MgSO₄, filtered and evaporated in vacuo to give a yellow oil. The compound was purified by normal phase preparative LC (regular SiOH, 50 μm, 24 g interchim, solid loading (Celite®), mobile phase gradient: from heptane/EtOAc 100/0 to 80/20 over 10 CV). The fractions containing compound were combined and evaporated in vacuo to give a white solid (723 mg).

The solid was purified by normal phase preparative LC (regular SiOH, 50 μm, 24 g interchim, solid loading (Celite®), mobile phase gradient: from heptane/DCM 100/0 to 50/50 over 10 CV). The fractions containing compound were combined and evaporated in vacuo to give Int 121.2 as a colorless oil (340 mg, 29%).

1H NMR (400 MHz, CDCl₃) δ (ppm): 6.74 (s, 2H), 5.64-5.53 (m, 1H), 2.46 (s, 3H)

LCMS (Method S): Rt=1.83 min; MS m/z [M+H]⁺=317.0

### Step 3 Preparation of Int 121.3

Int 121.2 (330 mg, 0.00104 mol) and K₂CO₃ (1.2 eq., 0.172 g, 0.00125 mol) were dissolved in DMF (5.00 mL) and purged with N₂. Then, chloromethyl methyl ether (1.87 eq., 0.180 mL, 0.00194 mol) was added dropwise and stirred for 18 h at rt. The mixture was diluted with EtOAc and washed with water. Organic layer was dried over MgSO₄, filtered and evaporated in vacuo. The compound was purified by normal phase preparative LC (regular SiOH, 50 μm, 12 g interchim, solid loading (Celite®), mobile phase gradient: from heptane/EtOAc 100/0 to 50/50 over 10 CV). The fractions containing compound were combined and evaporated in vacuo to give Int 121.3 as a colourless oil (345 mg, 88%). 1H NMR (500 MHz, CDCl₃) δ (ppm): 6.83 (s, 2H), 5.28 (s, 2H), 3.76 (q, J=7.1 Hz, 2H), 2.48 (s, 3H), 1.23 (t, J=7.1 Hz, 3H).

LCMS (Method S): Rt=1.61 min; MS m/z [M+H]⁺=375.0;

### Step 4 Preparation of Int 121.4

In a sealed flask, a mixture of Int121.3 (1 eq., 330 mg, 0.000877 mol), Pd(OAc)₂ (0.025 eq., 0.00492 g, 2.19e-5 mol) and CyJohnPhos (0.05 eq., 0.0154 g, 4.39e-5 mol) was purged with N₂ twice. Then 1,4-dioxane (4.00 mL) was added followed by triethylamine (3 eq., 0.266 g, 0.00263 mol) and pinacolborane (2 eq., 225 mg, 0.00175 mol), and the reaction was purged again with N₂ before being heated at 80° C. for 4 h.

The reaction mixture was cooled to rt, washed with water and extracted with EtOAc. The organic phase was dried over MgSO₄, filtered and concentrated to give a dark green solid. The compound was purified by normal phase preparative LC (regular SiOH, 30 μm, 24 g interchim, dry loading (Celite®), mobile phase gradient: from heptane/CH₂Cl₂ 100/0 to 50/50 over 10 CV). The fractions containing compound were combined and evaporated in vacuo to give Int121.4 as a yellow oil (180.7 mg, 55%).

H NMR (500 MHz, CDCl₃) δ (ppm): 6.74 (s, 1H), 6.66 (s, 1H), 5.17 (s, 2H), 3.71 (q, J=7.0 Hz, 2H), 2.35 (s, 3H), 1.38 (s, 12H), 1.20 (t, J=7.1 Hz, 3H)

### Step 5 Preparation of Int 121.5

Int 121.4 (1 eq., 137 mg, 0.000364 mol) was dissolved in dichloromethane (5.00 mL) and cooled to 0° C. Then, TFA (5 eq., 0.208 g, 0.00182 mol) was added, the reaction mixture was stirred at 0° C. for 30 min and allowed to warm up to rt and stirred for 2 h. The mixture was washed with water. Organic layer was separated, dried over MgSO₄, filtered and evaporated in vacuo to give Int 121.5 as a yellowish oil (110 mg, 95%).

1H NMR (500 MHz, CDCl₃) δ (ppm): 8.73 (s, 1H), 6.58-6.52 (m, 2H), 2.49 (s, 3H), 1.37 (s, 12H)

LCMS (Method S): Rt=1.76 min; MS m/z [M+H]⁺=317.2;

### 3,6-dimethyl-2-(4,4,5,5-tetramethyl-1,3,2-dioxaborolan-2-yl)phenol

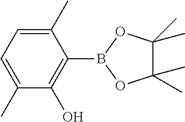 [SMILES: Cc1ccc(C)c(B2OC(C)(C)C(C)(C)O2)c1O]

### Step 1 Preparation of Int 141.1

2,5-dimethylphenol (1 eq., 1.00 g, 8.19 mmol, 1 eq) was dissolved in DCM (50 mL) and diisopropylamine (0.1 eq., 115 μL, 0.819 mmol) was added. A suspension of NBS (1 eq., 1.46 g, 8.19 mmol) in DCM (50 mL) was then added dropwise using a dropping funnel over the course of approximately 1 h; the clear mixture progressively took a deep yellow colour. The mixture was stirred for 1 h afterwards at room temperature, at which point the yellow colour had disappeared and the mixture was clear again.

The mixture was washed with aqueous HCl 1M (50 mL), water (50 mL), then dried and concentrated to give a transparent oil (1.52 g). The product was purified by normal phase preparative LC (regular SiOH, 50 μm, 40 g interchim, liquid loading (DCM), mobile phase gradient: from heptane/DCM 100/0 to 50/50 over 10 CV). The fractions containing compound were combined and evaporated in vacuo to give Int 141.1 as a colorless oil (1.18 g, 91% purity, 65% yield).

¹H NMR (500 MHz, CDCl₃) δ (ppm): 6.96 (d, J=7.6 Hz, 1H), 6.72 (d, J=7.6 Hz, 1H), 5.65 (s, 1H), 2.36 (s, 3H), 2.27 (s, 3H). Presence of di-brominated product (9% w/w).

LCMS (Method S): Rt=1.32 min; MS m/z [M+H]⁺=199.1/201.1

### Step 2 Preparation of Int 141.2

Int141.1 (1 eq., 840 mg, 91% purity, 3.80 mmol), bis(pinacolato)diboron (1.4 eq., 1.35 g, 5.32 mmol) and potassium acetate (2.3 eq., 858 mg, 8.74 mmol) were suspended in dioxane (21 mL) and the vial was purged under vacuum and backfilled with nitrogen 3 times. Tetrakis(triphenylphosphine)palladium(0) (0.01 eq., 43.9 mg, 0.038 mmol) was then added, the vial was purged under vacuum and backfilled with nitrogen 3 times, and the resulting orange suspension was stirred at 100° C. for at least 2 h and up to 66 h (the temperature probe shut down at some point over the week-end and the heating stopped).

Tetrakis(triphenylphosphine)palladium(0) (0.02 eq., 88.0 mg, 0.076 mmol) was added, the vial was purged and backfilled with nitrogen three times and the dark brown mixture was stirred at 100° C. for 18 h.

The mixture was left to cool to room temperature, then filtrated over Celite® and concentrated to give a pale brown oil (2024 mg) that was purified by normal phase preparative LC (regular SiOH, 50 μm, 80 g interchim, liquid loading (DCM), mobile phase gradient: from heptane/EtOAc 100/0 to 70/30 over 10 CV, UV detection at 254 and 301 nm) to give a white solid (564 mg).

The product was repurified by normal phase preparative LC (regular SiOH, 15 μm, 25 g interchim, liquid loading (DCM), mobile phase gradient: from heptane/DCM 100/0 to 50/50 over 10 CV, UV detection at 254 and 301 nm) to give Int 141.2 (373 mg, 40%).

¹H NMR (500 MHz, CDCl₃) δ (ppm): 8.65 (s, 1H), 7.00 (d, J=7.5 Hz, 1H), 6.53 (d, J=7.5 Hz, 1H), 2.38 (s, 3H), 2.11 (s, 3H), 1.28 (s, 12H)

LCMS (Method S): Rt=1.74 min; MS m/z [M+H]⁺=247.2;

### 2,4-dimethyl-6-(4,4,5,5-tetramethyl-1,3,2-dioxaborolan-2-yl)phenol

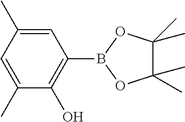 [SMILES: Cc1cc(C)c(O)c(B2OC(C)(C)C(C)(C)O2)c1]

### Preparation of Int 152.1

A vial was loaded with 2-bromo-4,6-dimethyl-phenol (1 eq., 500 mg, 0.00249 mol), bis(pinacolato)diboron (1.5 eq., 0.947 g, 0.00373 mol) and potassium acetate (4 eq., 0.976 g, 0.00995 mol), purged under vacuum and backfilled with nitrogen 3 times, then dioxane (20.0 mL) was added. tetrakis(triphenylphosphine)palladium(0) (0.05 eq., 0.144 g, 0.000124 mol) was then added, the vial was purged under vacuum and backfilled with nitrogen 3 times again, and the resulting orange mixture was stirred at 100° C. for 18 h, at which point it had turned dark brown.

The mixture was filtrated over Celite® and concentrated to give a brown oil (1365 mg) that was purified by normal phase preparative LC (regular SiOH, 50 μm, 40 g interchim, liquid loading (DCM), mobile phase gradient: from heptane/EtOAc 100/0 to 70/30 over 10 CV, UV detection at 254 and 301 nm) to give a white solid (443 mg).

The product was repurified by normal phase preparative LC (regular SiOH, 15 μm, 12 g interchim, liquid loading (DCM), mobile phase gradient: from heptane/DCM 100/0 to 50/50 over 10 CV, UV detection at 254 and 301 nm) to give Int152.1 as a white solid (290 mg, 47%).

1H NMR (400 MHz, CDCl3) δ (ppm): 7.82 (d, J=0.6 Hz, 1H), 7.27-7.24 (m, 1H), 7.07-7.04 (m, 1H), 2.23 (s, 3H), 2.21 (s, 3H), 1.36 (s, 12H).

LCMS (Method S): Rt=1.66 min; MS m/z [M+H]⁺=247.3

### 4,5-dimethyl-2-(4,4,5,5-tetramethyl-1,3,2-dioxaborolan-2-yl)phenol

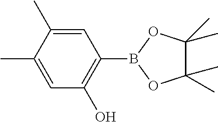

### Step 1 Preparation of Int 191.1

In a 1 L flask cooled by a water bath at room temperature, 3,4-dimethylphenol (1 eq., 5.00 g 0.0409 mol) was dissolved in DCM (250 mL) then diisopropylamine (0.1 eq., 0.414 g, 0.00409 mol) was added. A solution of NBS (1 eq., 7.28 g, 0.0409 mol) in DCM (250 nL) was then added dropwise using a dropping funnel over approximately 51 h (Note: this solution possibly degraded to bromine as it progressively turned from a clear suspension to a dark red mixture over several hours). The pale yellow mixture was then stirred for 18 h at room temperature. The mixture was washed with a solution of HCl 1 M (200 mL), water (200 mL) and brine (200 mL). dried and concentrated to give a yellow oil, which was purified. by normal phase preparative LC (regular SiOH, 50 μm, 220 g interchim, liquid loading (DCM), mobile phase gradient: from heptane/DCM 100/0 to 50/50 over 10 CV). The fractions containing compound were combined and evaporated in vacuo to give a white solid (3896 mg) The product was repurified by preparative chiral SFC (Stationary phase: Chiralpak® IG 20×250 mm, Mobile phase: CO₂/(iPrOH+0.3% iPrNH2) 95/5) to give In11.1 as a brown oil (806 mg, 10%).

¹H NMR (400 MHz, CDCl₃) δ (ppm): 7.02 (d J=8.2 Hz, 1H), 6.82 (d, J=8.2 Hz., 1H), 2.38 (s, 3H), 2.29 (s, 3H).

LCMS (Method S) Rt=1.28 min; MS m/z [M−H]⁻=199.0/201.0

### Step 2 Preparation of Int 191.2

In a vial, Int191.1 (1 eq., 400 mg, 0.00199 mol), bis(pinacolato)diboron (1.5 eq., 0.758 g, 0.00298 mol) and potassium acetate (4 eq., 0.781 g, 0.00796 mol) were suspended in 1,4-dioxane (12.5 mL). The vial was purged under vacuum and backfilled with nitrogen 3 times, then tetrakis(triphenylphosphine)palladium(0) (0.05 eq., 0.115 g, 9.95e-5 mol) was added, and the vial was purged again under vacuum and backfilled with nitrogen 3 times. The resulting ochre-orange suspension was stirred at 100° C. for 18 h, at which point it had turned to a dark brown mixture.

The mixture was filtrated over Celite® and concentrated to give a brown oil (1365 mg) that was purified by normal phase preparative LC (regular SiOH, 50 μm, 40 g interchim, liquid loading (DCM), mobile phase gradient: from heptane/EtOAc 100/0 to 70/30 over 10 CV, UV detection at 254 and 301 nm) to give Int191.1 as a white solid (343 mg, 77% pure, 54% yield).

¹H NMR (500 MHz, CDCl₃) δ (ppm): 8.47 (s, 1H), 7.11 (d, J=8.3 Hz, 1H), 6.64 (d, J=8.3 Hz, 1H), 2.43 (s, 3H), 2.18 (s, 3H), 1.37 (s, 12H).

LCMS (Method S): Rt=1.64 min; MS m/z [M−H]⁻=247.1;

### 5-isopropyl-3-methyl-2-(4,4,5,5-tetramethyl-1,3,2-dioxaborolan-2-yl)phenol (Int 274.2)

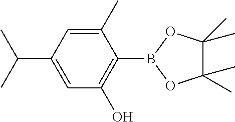 [SMILES: Cc1cc(C(C)C)cc(O)c1B1OC(C)(C)C(C)(C)O1]

### Step 1 2-iodo-5-isopropyl-3-methyl-phenol (Int 274.1)

5-isopropyl-3-methylphenol (1000 mg, 6,657 mmol, 100 mass %) was solubilized in toluene (60 ml) and THF (4 ml) under inert atmosphere. Sodium hydride (2.1 equiv., 13.9797 mmol, 60 mass %) were add at rt and stirred for 30 mn. The mixture was cooled at 0° C. before the dropwise addition of a solution of iodine (B, 1 equiv., 6.657 mmol, 100 mass %) in toluene (44 ml). Stirring was continued for 1 h30 at rt. The reaction mixture was poured into a solution of water and NaHSO₃ (pH=5) and extracted with AcOEt. The organic layer was wash with brine, dried over magnesium sulfate, and concentrated under reduced pressure. The residue was purified by column chromatography on silica gel (eluant heptane/AcOt 15% 40 mn) to obtain 200 mg of Int 274.1 as a colorless oil, yield 11%

LCMS (Method L): Rt=1.13 min; MS m/z [M−H]⁻=275

### Step 2 5-isopropyl-3-methyl-2-(4,4,5,5-tetramethyl-1,3,2-dioxaborolan-2-yl)phenol (Int 274.2)

Int 274.1 (754 mg, 2.7308 mmol, 100 mass %) were solubilized in dioxane (14 mL, 100 mass %) under Ar atmosphere and cooled down to 0° C. After addition of Triethylamine (2.30 equiv., 2.6766 mmol, 100 mass %), the mixture was stirred for 5 mn. 4,4,5,5-tetramethyl-1,3,2-dioxaborolane (D, 2.8 equiv., 7.6461 mmol, 100 mass %) were add dropwise, stirred for 5 mn before the add of Palladium(II) acetate (0.0500 equiv., 0.1365 mmol, 100 mass %) and 2-(dicyclohexylphosphino)biphenyl (0.100 equiv., 0.2731 mmol, 100 mass %). The mixture was heated at 75° C. for 30 mn.

The reaction mixture was poured into water and extracted with AcOEt. The organic layer was wash with brine, dried over magnesium sulfate, and concentrated under reduced pressure. The residue was purified by column chromatography on silica gel (eluant heptane/AcOt 15% 40 mn) to obtain 440 mg of Int 274.2 (5-isopropyl-3-methyl-2-(4,4,5,5-tetramethyl-1,3,2-dioxaborolan-2-yl)phenol) as an orange solid used without further purification for the next step.

LCMS (Method L): Rt=1.44 min; MS m/z [M−H]⁻=275

### 5-(difluoromethyl)-3-methyl-2-(4,4,5,5-tetramethyl-1,3,2-dioxaborolan-2-yl)phenol (Int 134.2)

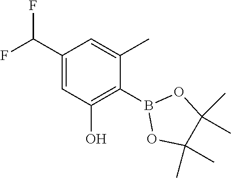 [SMILES: Cc1cc(C(F)F)cc(O)c1B1OC(C)(C)C(C)(C)O1]

### Step 1 5-(difluoromethyl)-2-iodo-3-methyl-phenol (Int 134.1) and 3-(difluoromethyl)-2-iodo-5-methyl-phenol (Int 153.1)

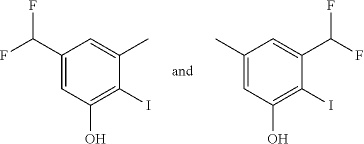

To a cooled (0-5° C.) suspension of NaH (60% in oil—39.8 mmol, 1.6 g) in 80 ml of toluene, was slowly added a solution of 3-(difluoromethyl)-5-methyl-phenol (19 mmol, 3 g) in 20 ml of toluene while keeping temperature below 10° C. The mixture was stirred for 45 min at 0-5° C., then a solution of iodine (19 mmol, 4.8 g) in 80 ml of toluene was slowly added while keeping temperature at −5° C. The mixture was stirred at 0° C. for 5 min, then quenched with 100 ml of an aqueous 5% citric acid solution and 100 ml of diethylether. The organic layer was successively washed with aqueous 10% sodium bisulfite solution and water then dried over Na₂SO₄, filtered and concentrated under reduced pressure. The residue was purified by flash chromatography on silica gel eluting with a gradient of 0 to 100% of diethylether in petroleum ether to give successively 5-(difluoromethyl)-2-iodo-3-methyl-phenol (Int 134.1) as a colorless liquid (2 g, 37% yield) and 3-(difluoromethyl)-2-iodo-5-methyl-phenol (Int 153.2) as a white solid (1.36 g, 25% yield).

### LCMS (Method L)

Int 134.1 Rt=1.26 min; MS m/z [M−H]+ 283
Int 153.1 Rt=1.20 min; MS m/z [M−H]+ 283

### Step 2 5-(difluoromethyl)-3-methyl-2-(4,4,5,5-tetramethyl-1,3,2-dioxaborolan-2-yl)phenol (Int 134.2)

Following procedure of Int 96.2, 5-(difluoromethyl)-2-iodo-3-methyl-phenol Int 134.1 (2.46 mmol, 700 mg) gave the title compound as colourless oil (321 mg, 46% yield).

LCMS (Method X): Rt=1.63 min; MS m/z [M+H]⁺ 285

### 3-(difluoromethyl)-5-methyl-2-(4,4,5,5-tetramethyl-1,3,2-dioxaborolan-2-yl)phenol (Int 153.2)

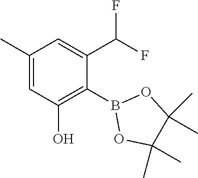

Following procedure of Int 96.2, 3-(difluoromethyl)-2-iodo-5-methyl-phenol (Int 153.1, 1.76 mmol, 500 mg) gave the title compound as an orange oil (not isolated and used crude at the next step).

LCMS (Method L): Rt=1.54 min; MS m/z [M−H]⁺ 283.

### 5-chloro-3-methyl-2-(4,4,5,5-tetramethyl-1,3,2-dioxaborolan-2-yl)phenol

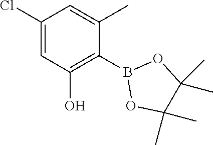 [SMILES: Cc1cc(Cl)cc(O)c1B1OC(C)(C)C(C)(C)O1]

### Step 1 5-chloro-2-iodo-3-methyl-phenol (Int 133.1) and 3-chloro-2-iodo-5-methyl-phenol (Int 172.1)

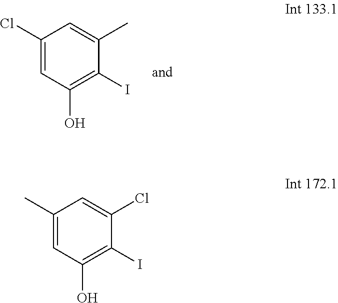

Following procedure of Int 134.1 and Int 153.1, 3-chloro-5-methyl-phenol (140 mmol, 20 g) gave the title compounds 5-chloro-2-iodo-3-methyl-phenol (Int 133.1) (19.08 g, 51% yield) and 3-chloro-2-iodo-5-methyl-phenol (Int 172.1) (4.7 g, 9% yield) as white solids.

### LCMS (Method L):

Int 133.1 Rt=1.05 min; MS m/z [M−H]+ 267
Int 172.1 Rt=1.00 min; MS m/z [M−H]+ 267

### Step 2 5-chloro-3-methyl-2-(4,4,5,5-tetramethyl-1,3,2-dioxaborolan-2-yl)phenol

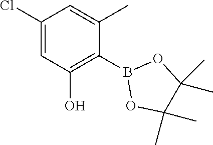 [SMILES: Cc1cc(Cl)cc(O)c1B1OC(C)(C)C(C)(C)O1]

To a cooled (10-15° C.) solution of 5-chloro-2-iodo-3-methyl-phenol (Int 133.1, 32.1 mmol, 8.63 g) and triethylamine (64.3 mmol, 8.77 ml) in 140 ml of anhydrous dioxanne under argon, was slowly added 4,4,5,5-tetramethyl-1,3,2-dioxaborolane (70.7 mmol, 10.1 ml) while keeping temperature below 15° C. (strong gas evolution). The mixture was stirred for 30 min at rt until complete solubilization, then diacetoxypalladium (1.61 mmol, 361 mg) and dicyclohexyl-(2-phenylphenyl)phosphane (3.21 mmol, 1.13 g) were added. The mixture was stirred at 50° C. for 30 min, then quenched with 250 ml of diethylether, 250 ml of ethyl acetate and 200 ml of an aqueous 5% citric acid solution. The organic layer was washed with water, dried over Na₂SO₄, filtered and concentrated under reduced pressure. The residue was purified by flash chromatography on silica gel eluting with 20% of diethylether in pentane to give the title compound as a colorless oil (7 g, 71% yield).

LCMS (Method L): Rt=1.38 min; MS m/z [M−H]⁺ 267

### 3-chloro-5-methyl-2-(4,4,5,5-tetramethyl-1,3,2-dioxaborolan-2-yl)phenol

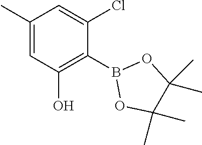

Following procedure of Int 96.2, 3-chloro-2-iodo-5-methyl-phenol (Int 172.1, 2.23 mmol, 600 mg) gave the title compound an orange oil (596 mg, 99% yield).

LCMS (Method L): Rt=1.23 min; MS m/z [M+H]⁺ 269.

### 5-cyclopropyl-3-methyl-2-(4,4,5,5-tetramethyl-1,3,2-dioxaborolan-2-yl)phenol

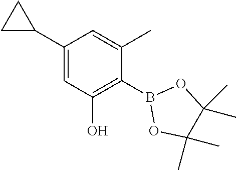 [SMILES: Cc1cc(C2CC2)cc(O)c1B1OC(C)(C)C(C)(C)O1]

### Int 204.1: 1-cyclopropyl-3-iodo-5-methoxy-benzene

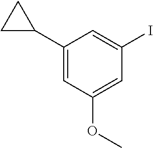

To a cooled (0° C.) solution of 3-cyclopropyl-5-methoxy-aniline (12.3 mmol, 2 g) in 80 ml of an aqueous 4N HCl solution and 65 ml of acetonitrile, was slowly added a solution of sodium nitrite (13.5 mmol, 930 mg) in 2 ml of water while keeping temperature below 5° C. The mixture was stirred for 30 min at 0-5° C. Then was slowly added a solution of sodium iodide (24.5 mmol, 3.67 g) and the mixture was stirred at rt for 1 h30. Until end of gas evolution then quenched with 50 ml of an aqueous 10% sodium hydrogenosulfite solution, 20 ml of diethylether and 80 ml of pentane. The organic layer was washed with water, dried over Na₂SO₄, filtered and concentrated under reduced pressure. The residue was purified by flash chromatography on silica gel eluting with a gradient of 0 to 100% of diethylether in pentane to give the title compound as a colorless oil (2.67 g, 75% yield).

LCMS (Method V): Rt=1.30 min; MS m/z [M+H]⁺ 275;

### Int 204.2: 1-cyclopropyl-3-methoxy-5-methyl-benzene

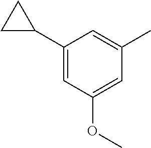

To an argon degassed solution of 1-cyclopropyl-3-iodo-5-methoxy-benzene (9.74 mmol, 2.67 g) in 50 ml of 1,4-dioxanne, was added 2,4,6-trimethyl-1,3,5,2,4,6-trioxatriborinane (24.4 mmol, 3.4 ml) and potassium carbonate (24.4 mmol, 3.37 g). The suspension was stirred for 5 min at rt and then was added [1,1′-bis(diphenylphosphino)ferrocene]dichloropalladium(II) (0.49 mmol, 356 mg) and the mixture was stirred at 100° C. for 1 h. After cooling to rt, the mixture was diluted with 20 ml of diethylether and 80 ml of pentane and filtered over a Decalite® pad. The cake was rinsed with 20 ml of diethylether and the pooled organic layers are concentrated under reduced pressure. The residue was purified by flash chromatography on silica gel eluting with a gradient of 0 to 10% of diethylether in pentane to give the title compound as a yellow oil (1.2 g, 76% yield).

LCMS (Method V): Rt=1.18 min; MS m/z [M+H]⁺ 163

### Int 204.3: 3-cyclopropyl-5-methyl-phenol

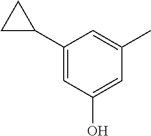

To a cooled (−10° C.) solution of 1-cyclopropyl-3-methoxy-5-methyl-benzene (8.51 mmol, 1.38 g) in 60 ml of dichloromethane under argon, was slowly added a TN dichloromethane boron tribromide solution (8.51 mmol, 8.51 ml). The solution was stirred for 1 h at −10° C. and 1 h at 0° C. then quenched under strong stirring with 50 ml of a saturated sodium hydrogenocarbonate aqueous solution, 50 ml of water and 100 ml of diethylether. The organic layer was washed with water, dried over Na₂SO₄, filtered, and concentrated under reduced pressure. The residue was purified by flash chromatography on silica gel eluting with a gradient of 0 to 60% of diethylether in pentane to give the title compound as a pale yellow oil (770 mg, 61% yield).

LCMS (Method V): Rt=0.88 min; MS m/z [M+H]⁺ 149

### Int 204.4: 5-cyclopropyl-2-iodo-3-methyl-phenol

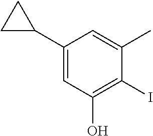

Following procedure of Int 134.1 and Int 153.1, 3-cyclopropyl-5-methyl-phenol (5.2 mmol, 770 mg) gave the title compound (370 mg, 26% yield) as white solid.

LCMS (Method V): Rt=0.88 min; MS m/z [M−H]⁺ 273.

### 5-cyclopropyl-3-methyl-2-(4,4,5,5-tetramethyl-1,3,2-dioxaborolan-2-yl)phenol

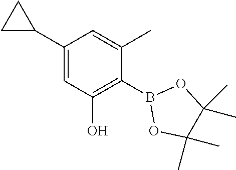 [SMILES: Cc1cc(C2CC2)cc(O)c1B1OC(C)(C)C(C)(C)O1]

Following procedure of Int 96.2, 5-cyclopropyl-2-iodo-3-methyl-phenol (1.35 mmol, 370 mg) gave the title compound (not isolated and used crude at the next step).

LCMS (Method V): Rt=1.41 min; MS m/z [M+H]⁺ 275

### Int 324.2: 5-(4,4,5,5-tetramethyl-1,3,2-dioxaborolan-2-yl)indan-4-ol

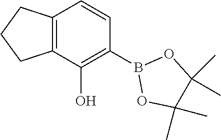 [SMILES: CC1(C)OB(c2ccc3c(c2O)CCC3)OC1(C)C]

### Int 324.1: 5-iodoindan-4-ol

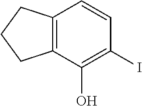 [SMILES: Oc1c(I)ccc2c1CCC2]

Following procedure of Int 353.1 using indan-4-ol (14.9 mmol, 2 g), the title compound was obtained as a colourless oil (700 mg, 18% yield).

LCMS (Method V): Rt=1.15; MS m/z [M−H]⁺=259

### Int 324.2: 5-(4,4,5,5-tetramethyl-1,3,2-dioxaborolan-2-yl)indan-4-ol

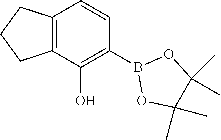 [SMILES: CC1(C)OB(c2ccc3c(c2O)CCC3)OC1(C)C]

Following procedure of Int 96.2 using 5-iodoindan-4-ol (2.69 mmol, 700 mg), the title compound was obtained as a brown oil (588 mg, 84% yield)

LCMS (Method V): Rt=1.47; MS m/z [M+H]⁺=261

### Int 380.3: 3-ethyl-2-(4,4,5,5-tetramethyl-1,3,2-dioxaborolan-2-yl)-5-(trifluoromethyl) phenol

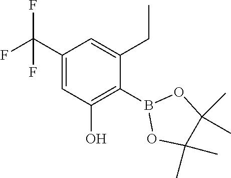 [SMILES: CCc1cc(C(F)(F)F)cc(O)c1B1OC(C)(C)C(C)(C)O1]

### Int 380.1: 3-ethyl-5-(trifluoromethyl)phenol

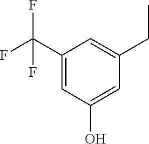

Under Ar, to a solution of 3-bromo-5-(trifluoromethyl)phenol (8.3 mmol, 2 g) in 20 ml of 1,4-dioxane were added potassium phosphate tribasic (16.6 mmol, 3.5 g), ethylboronic acid (9.96 mmol, 736 mg and [1,1′-bis(diphenylphospino)ferrocene]dichloropalladium(II) (0.41 mmol, 304 mg). The mixture was stirred at 110° C. for 3 h. After cooling down to rt, the mixture was concentrated under reduced pressure and the residue was purified by flash chromatography on silica gel eluting with a gradient of 0 to 50% of EtOAc in cyclohexane to give the title compound as a yellow oil (1.05 g, 67% yield).

LCMS (Method V): Rt=0.94; MS m/z [M−H]⁺=189

### Int 380.2: 3-ethyl-2-iodo-5-(trifluoromethyl)phenol

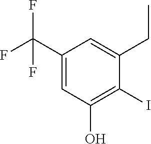

Under argon, to a cooled (0-5° C.) suspension of NaH (60% in oil—11.6 mmol, 464 mg) in ml of anhydrous toluene was slowly added a solution of 3-ethyl-5-(trifluoromethyl)phenol (5.52 mmol, 1.05 g) in 15 ml of toluene while keeping temperature below 10° C. The mixture was stirred for 45 min at 0-5° C., then the mixture was cooled down to −5° C. and a solution of iodine (5.52 mmol, 1.4 g) in 15 ml of toluene was slowly added while keeping temperature at −5° C. The mixture was then stirred at 0° C. for 10 min, then quenched with 100 ml of a TN aqueous HCl solution and 100 ml of EtOAc. The organic layer was successively washed with 100 ml of an aqueous 10% sodium bisulfite solution and water then dried over Na₂SO₄, filtered and concentrated under reduced pressure. To the residue was added a mixture of 10 ml of N,N-dimethylformamide and 2 ml of formic acid and the resulting solution was stirred at 160° C. for 3 h, then cooled down to rt. The mixture was then diluted in 150 ml of diethylether and washed with 3×100 ml of water. The organic layer was dried over Na₂SO₄, filtered and concentrated under reduced pressure. The residue was purified by flash chromatography on silica gel eluting with a gradient of 0 to 100% of diethylether in cyclohexane to give the title compound as a colorless liquid (415 mg, 37% yield).

LCMS (Method V): Rt=1.25; MS m/z [M−H]⁺=315

### Int 380.3: 3-ethyl-2-(4,4,5,5-tetramethyl-1,3,2-dioxaborolan-2-yl)-5-(trifluoromethyl) phenol

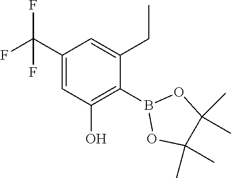 [SMILES: CCc1cc(C(F)(F)F)cc(O)c1B1OC(C)(C)C(C)(C)O1]

Following procedure of Int 96.2, 3-ethyl-2-iodo-5-(trifluoromethyl)phenol (1.31 mmol, 415 mg) gave the title compound as an orange oil (not isolated and used crude for the next step).

LCMS (Method V): Rt=1.43; MS m/z [M−H]⁺=315

### Int 381.3: 5-chloro-3-ethyl-2-(4,4,5,5-tetramethyl-1,3,2-dioxaborolan-2-yl)phenol

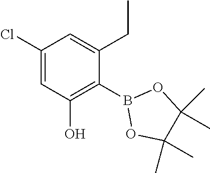 [SMILES: CCc1cc(Cl)cc(O)c1B1OC(C)(C)C(C)(C)O1]

### Int 381.1: 3-chloro-5-ethyl-phenol

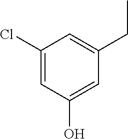

Following procedure of Int 380.1, 3-bromo-5-chloro-phenol (4.8 mmol, 1 g) gave the title compound as a yellow liquid (542 mg, 72% yield).

LCMS (Method V): Rt=1.06; [M−H]⁺=155

### Int 381.2: 5-chloro-3-ethyl-2-iodo-phenol

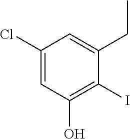

Following procedure of Int 380.2, 3-chloro-5-ethyl-phenol (2.82 mmol, 442 mg) gave the title compound as a colourless oil (180 mg, 21% yield)

LCMS (Method V): Rt=1.21; MS m/z [M−H]⁺=281

### Int 381.3: 5-chloro-3-ethyl-2-(4,4,5,5-tetramethyl-1,3,2-dioxaborolan-2-yl)phenol

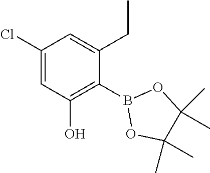 [SMILES: CCc1cc(Cl)cc(O)c1B1OC(C)(C)C(C)(C)O1]

Following procedure of Int 96.2, 5-chloro-3-ethyl-2-iodo-phenol (0.64 mmol, 180 mg) gave the title compound as a brown oil (not isolated and used crude for the next step).

LCMS (Method V): Rt=153; MS m/z [M+H]⁺=283

### Int 354.1: (2-hydroxy-6-methyl-phenyl)boronic acid

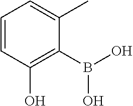

Under argon, at 0° C., to a solution of (2-methoxy-6-methyl-phenyl)boronic acid (4.28 mmol, 710 mg) in 15 ml of anhydrous DCM was added dropwise a 1N boron tribromide solution in DCM (10.7 mmol, 10.7 ml) and the mixture was stirred at 0° C. for 30 min, then quenched with 30 ml of EtOAc and 20 ml of ice-water under strong stirring. The organic layer was washed with 10 ml of water, dried over Na₂SO₄, filtered and concentrated under reduced pressure. The residue was purified by flash chromatography on silica gel eluting with a gradient of 0 to 10% of MeOH in DCM to give the title compound as a yellow solid (330 mg, 51% yield).

LCMS (Method V): Rt=0.48; MS m/z [M−H]⁺=151

### Int 353.2: 6-fluoro-3-methyl-2-(4,4,5,5-tetramethyl-1,3,2-dioxaborolan-2-yl)phenol

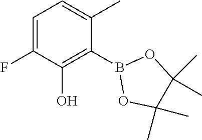 [SMILES: Cc1ccc(F)c(O)c1B1OC(C)(C)C(C)(C)O1]

### Int 353.1: 6-fluoro-2-iodo-3-methyl-phenol

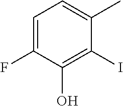

Under argon, to a cooled (0-5° C.) suspension of NaH (60% in oil—33.33 mmol, 1.33 g) in 50 ml of toluene was slowly added a solution of 2-fluoro-5-methyl-phenol (15.86 mmol, 2 g) in 25 ml of toluene while keeping temperature below 10° C. The mixture was stirred for 45 min at 0-5° C. The mixture was cooled down to −10° C., then a solution of iodine (15.86 mmol, 3 g) in 50 ml of toluene was slowly added while keeping temperature below −10° C. The resulting mixture was stirred at 0° C. for 10 min, then quenched with 100 ml of diethylether, 50 ml of an aqueous 2N HCl solution and 10 ml of an aqueous sodium bisulfite 10% solution. The organic layer was washed twice with water, then dried over Na₂SO₄, filtered and concentrated under reduced pressure. The residue was purified by flash chromatography on silica gel eluting with 5% of diethylether in pentane to give the title compound as a white solid (2.37 g, 55% yield).

LCMS (Method V): Rt=1.01; MS m/z [M−H]⁺=251

### Int 353.2: 6-fluoro-3-methyl-2-(4,4,5,5-tetramethyl-1,3,2-dioxaborolan-2-yl)phenol

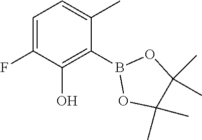 [SMILES: Cc1ccc(F)c(O)c1B1OC(C)(C)C(C)(C)O1]

Following procedure of Int 96.2, 6-fluoro-2-iodo-3-methyl-phenol (9.4 mmol, 2.37 g) gave the title compound as a brown oil (not isolated and used crude for the next step).

LCMS (Method V): Rt=153; MS m/z [M+H]⁺=283

### Int 358.2: (2-ethyl-6-hydroxy-phenyl)boronic acid

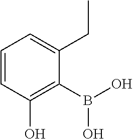

### Int 358.1: 2-(2-ethyl-6-methoxy-phenyl)-4,4,5,5-tetramethyl-1,3,2-dioxaborolane

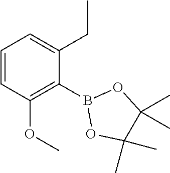 [SMILES: CCc1cccc(OC)c1B1OC(C)(C)C(C)(C)O1]

To a solution of 2-bromo-1-ethyl-3-methoxy-benzene (23.25 mmol, 5 g) in 50 ml of 1,4-dioxane was added 4,4,5,5-tetramethyl-2-(4,4,5,5-tetramethyl-1,3,2-dioxaborolan-2-yl)-1,3,2-dioxaborolane (34.87 mmol, 8.85 g) and anhydrous potassium acetate (69.74 mmol, 6.84 g). The resulting mixture was degazed under stirring by argon bubbling for 10 min. Then was added [1,1′bis(diphenylphosphino)ferrocene]dichloropalladium(II) (0.93 mmol, 680 mg) and the mixture was stirred at 100° C. for 13 h under argon. After cooling down to rt, the mixture was diluted with 100 ml of diethylether and 100 ml of water and filtered over a pad of decalite. The cake was rinsed with 50 ml of diethylether and the organic layers were pooled and washed with 2×50 ml of water, then dried over Na₂SO₄, filtered and concentrated under reduced pressure. The residue was purified by flash chromatography on silica gel eluting with a gradient of 0 to 50% of diethylether in pentane to give the title compound (1.77 g, 34% yield) as a colourless oil.

LCMS (Method V): Rt=1.29; MS m/z [M+H]⁺=263

### Int 358.2: (2-ethyl-6-hydroxy-phenyl)boronic acid

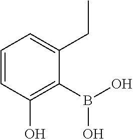

Under argon, at −10° C., to a solution of 2-(2-ethyl-6-methoxy-phenyl)-4,4,5,5-tetramethyl-1,3,2-dioxaborolane (1.63 mmol, 427 mg) in 15 ml of anhydrous DCM was dropwise added a 1N boron tribromide solution in DCM (4.07 mmol, 4.07 ml) and the mixture was stirred at −10° C. for 2 h. Then 30 ml of diethylether and 20 ml of a 1% sodium bisulfite aqueous solution were added under strong stirring. The organic layer was washed with 2×10 ml of water, dried over Na₂SO₄, filtered and concentrated under reduced pressure to give the title compound as a yellow solid (270 mg, 100% yield).

LCMS (Method V): Rt=0.58; MS m/z [M−H]⁺=165

### Int 324.2: 6-fluoro-3-methyl-2-(4,4,5,5-tetramethyl-1,3,2-dioxaborolan-2-yl)phenol

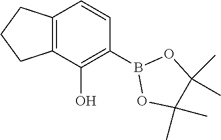 [SMILES: CC1(C)OB(c2ccc3c(c2O)CCC3)OC1(C)C]

### Int 324.1: 5-iodoindan-4-ol

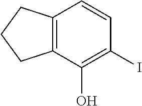 [SMILES: Oc1c(I)ccc2c1CCC2]

Following procedure of Int 353.1, indan-4-ol (14.9 mmol, 2 g) gave the title compound as a colourless oil (700 mg, 18% yield).

LCMS (Method V): Rt=1.15; MS m/z [M−H]⁺=259

### Int 324.2: 6-fluoro-3-methyl-2-(4,4,5,5-tetramethyl-1,3,2-dioxaborolan-2-yl)phenol

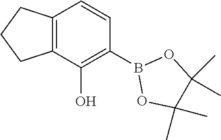 [SMILES: CC1(C)OB(c2ccc3c(c2O)CCC3)OC1(C)C]

Following procedure of Int 96.2, 5-iodoindan-4-ol (2.69 mmol, 700 mg) gave the title compound as a brown oil (588 mg, 84% yield)

LCMS (Method V): Rt=1.47; MS m/z [M+H]⁺=261

### Int 508.3: 5-[2-[tert-butyl(dimethyl)silyl]ethynyl]-3-methyl-2-(4,4,5,5-tetramethyl-1,3,2-dioxaborolan-2-yl)phenol

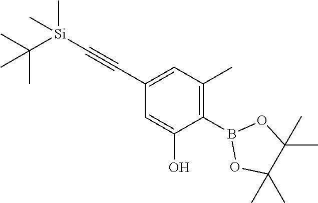 [SMILES: Cc1cc(C#C[Si](C)(C)C(C)(C)C)cc(O)c1B1OC(C)(C)C(C)(C)O1]

### Int 508.1: 3-[2-[tert-butyl(dimethyl)silyl]ethynyl]-5-methyl-phenol

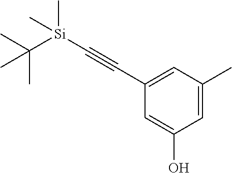 [SMILES: Cc1cc(O)cc(C#C[Si](C)(C)C(C)(C)C)c1]

To a solution of 3-bromo-5-methyl-phenol (2.00 g, 10.7 mmol) in 20 ml of DMF were added tert-butyl-ethynyl-dimethyl-silane (5.99 mL, 0.0321 mol) and NEt₃ (4.46 mL, 32.1 mmol. The mixture was purged with N₂, then PdCl₂dppf (873 mg, 1.07 mol) and CuI (204 mg, 1.07 mmol) were added. The mixture was purged again with N₂ then stirred at 80° C. for 3 h. After cooling down to rt, the mixture was concentrated under reduced pressure. The residue was purified by flash chromatography on silica gel using a gradient of heptane/EtOAc (from 100/0 to 80/20, then isocratic), to give the title compound as a dark orange oil (1.73 g, 66%).

¹H NMR (400 MHz, DMSO) δ (ppm): 9.40 (s, 1H), 6.56 (td, J=1.4, 0.7 Hz, 1H), 6.47 (ddd, J=2.4, 1.6, 0.8 Hz, 1H), 6.46-6.44 (m, 1H), 2.05 (s, 3H), 0.81 (s, 9H), 0.00 (s, 6H).

### Int 508.2 and Int 508.2bis: 3-[2-[tert-butyl(dimethyl)silyl]ethynyl]-2-iodo-5-methyl-phenol (Int 508.2) and 5-[2-[tert-butyl(dimethyl)silyl]ethynyl]-2-iodo-3-methyl-phenol (Int 508.2bis)

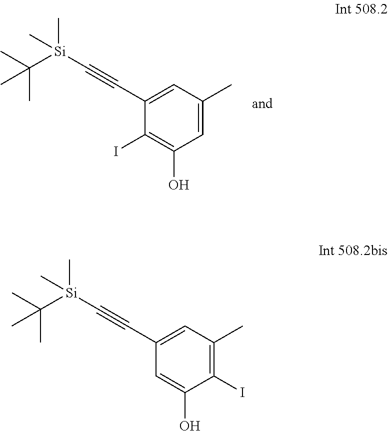

At 0° C., to a solution of 3-[2-[tert-butyl(dimethyl)silyl]ethynyl]-5-methyl-phenol (1.73 g, 0.00702 mol) in 70 ml of toluene was added NaH (60% in mineral oil) (60.0%, 0.562 g, 0.0140 mol). The mixture was stirred for 10 min then 12 (1.78 g, 0.00702 mol) was added. The reaction mixture was allowed to warm to rt and stirred for 18 h. The reacting mixture was diluted with EtOAc then quenched with a saturated aqueous solution of NH₄Cl. The aqueous layer was separated and extracted with EtOAc. The combined organic layers were dried over MgSO₄, filtered and concentrated under reduced pressure. The residue was purified by flash chromatography on silica gel using a gradient of heptane/DCM (from 100/0 to 80/20, then isocratic), then repurified by preparative chiral SFC (Stationary phase: Chiralpak® AD-3 4.6×100 mm, Mobile phase: CO₂/MeOH 90/10) to give 3-[2-[tert-butyl(dimethyl)silyl]ethynyl]-2-iodo-5-methyl-phenol (Int 508.2) as a white solid (1.02 g, 39%) and 5-[2-[tert-butyl(dimethyl)silyl]ethynyl]-2-iodo-3-methyl-phenol (Int 508.2bis) as a yellow oil (173 mg, 7%).

### Int 508.2

1H NMR (400 MHz, DMSO) δ (ppm): 10.37 (s, 1H), 6.73 (d, J=1.8 Hz, 1H), 6.56 (d, J=1.9 Hz, 1H), 2.18 (s, 3H), 0.81 (s, 9H), 0.00 (s, 6H).

### Int 508.2bis

1H NMR (400 MHz, DMSO) δ (ppm): 10.26 (s, 1H), 6.64 (dd, J=1.9, 0.8 Hz, 1H), 6.51 (dd, J=2.0, 0.8 Hz, 1H), 2.00 (s, 3H), 0.82 (s, 9H), 0.00 (s, 6H).

### Int 508.3: 5-[2-[tert-butyl(dimethyl)silyl]ethynyl]-3-methyl-2-(4,4,5,5-tetramethyl-1,3,2-dioxaborolan-2-yl)phenol

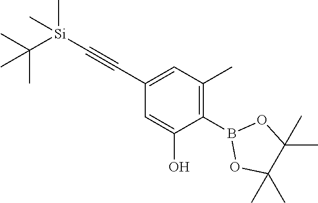 [SMILES: Cc1cc(C#C[Si](C)(C)C(C)(C)C)cc(O)c1B1OC(C)(C)C(C)(C)O1]

At 0° C., to a solution of 5-[2-[tert-butyl(dimethyl)silyl]ethynyl]-2-iodo-3-methyl-phenol (Int 508.2bis—140 mg, 0.376 mmol) and NEt₃ (0.367 mL, 2.63 mmol) in 5 ml of dioxane was added dropwise HBPin (0.273 mL, 1.88 mmol). The vial was purged with N₂, then Pd(OAc)₂ (8.44 mg, 3.76e-5 mol) and CyJohnPhos (26.4 mg, 7.52e-5 mol) were added. The solution was purged again with N₂ then stirred at 80° C. for 1 h. After cooling down to rt, the mixture was filtered and concentrated under reduced pressure. The residue was purified by flash chromatography on silica gel using heptane 100% to give the title compound as a white solid (65 mg, 46%).

¹H NMR (400 MHz, DMSO) δ (ppm): 9.32 (s, 1H), 6.69 (s, 1H), 6.60 (s, 1H), 2.21 (s, 3H), 1.29 (s, 12H), 0.95 (s, 9H), 0.15 (s, 6H).

### Int 509.2: 5-methyl-2-(4,4,5,5-tetramethyl-1,3,2-dioxaborolan-2-yl)-3-(trifluoromethyl) phenol

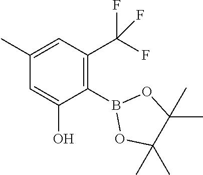

### Int 509.1: 2-iodo-5-methyl-3-(trifluoromethyl)phenol

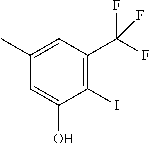

At 0° C., under N₂, to a mechanically stirred solution of 3-methyl-5-(trifluoromethyl)phenol (10.0 g, 0.0568 mol) in 200 ml of toluene, was slowly added NaH (60% in mineral oil) (2.27 g, 0.0568 mol) and the mixture was stirred for 30 min. Then 12 (14.4 g, 0.0568 mol) was added and the mixture was stirred at 0° C. for 1.5 h. The reaction mixture was warmed to rt and acidified with aqueous HCl 1M until pH=5. The resulting solution was extracted with EtOAc (3 times). The combined organic layers were washed with a saturated aqueous solution of Na₂S₂O₃, dried over MgSO₄, filtered and concentrated under reduced pressure. The residue was purified by flash chromatography on silica gel using heptane/DCM (from 100/0 to 80/20) to give 10 g of a colorless oil which 5 g was purified by preparative chiral SFC (Stationary phase: Chiralpak® 2EP 20×250 mm, Mobile phase: CO₂/MeOH 85/15) to afford the title compound (635 mg).

¹H NMR (400 MHz, DMSO-D₆) δ (ppm): 10.85 (s, 1H), 7.03 (d, J=1.8 Hz, 1H), 6.94 (d, J=1.9 Hz, 1H), 2.27 (s, 3H).

### Int 509.2: 5-methyl-2-(4,4,5,5-tetramethyl-1,3,2-dioxaborolan-2-yl)-3-(trifluoromethyl)phenol

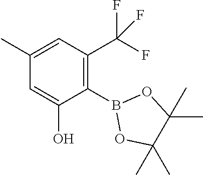

Under N₂, in a microwave vial, to a solution of 2-iodo-5-methyl-3-(trifluoromethyl)phenol (100 mg, 3.31 mmol) and Et₃N (0.235 g, 0.00232 mol) in 3 ml of 1,4-dioxane dry was slowly added HBPin (0.212 g, 0.00166 mol). the reaction mixture was then purged with N₂. Then palladium (II) diacetate (0.00372 g, 1.66e-5 mol) and CyJohnPhos (0.0116 g, 3.31e-5 mol) were added. The vial was purged again with N₂, then sealed and heated at 80° C. overnight. After cooling down to rt, the mixture was filtered and concentrated under reduce pressure. The residue was purified by flash chromatography on silica gel using heptane/EtOAc (from 100/0 to 60/40), then DCM/MeOH (100/0 to 95/5) to give the title compound as a pale yellow solid (200 mg, quant., 78% purity).

LCMS (Method T): Rt=7.03 min; MS m/z [M−H]⁻=301

### Int 360.6: 2-benzyl-6,7-dihydro-5H-pyrazolo[4,3-b][1,4]oxazine

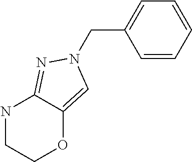 [SMILES: c1ccc(Cn2cc3c(n2)NCCO3)cc1]

### Int 360.1: 3-bromo-4-methoxy-1H-pyrazole

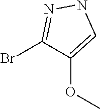

To a solution of 4-methoxy-1H-pyrazole (1.00 g, 0.0102 mol) in 80 ml of DMF was added NBS (1.81 g, 0.0102 mol) and the mixture was stirred at rt for 2 h. The mixture was quenched with a saturated aqueous solution Na₂S₂O₃ then extracted twice with DCM. The combined organic layers were washed with a saturated aqueous solution of NaHCO₃, dried over MgSO₄, filtered and concentrated under reduced pressure to give the title compound (1.03 g, 57%) which was used without further purification in the next step.

1H NMR (500 MHz, DMSO) δ (ppm): 12.71 (s, 1H), 7.57 (s, 1H), 3.71 (s, 3H) (+ remaining DMF)

### Int 360.2: 1-benzyl-3-bromo-4-methoxy-pyrazole

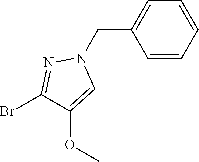

To a stirred solution of 3-bromo-4-methoxy-1H-pyrazole (830 mg, 0.00469 mol) and benzyl bromide (0.668 mL, 0.00563 mol) in 40 ml of DMF was added cesium carbonate (1.83 g, 0.00563 mol) and the solution was stirred at rt for 1 h. Additional benzyl bromide (0.278 mL, 0.00234 mol) and cesium carbonate (0.764 g, 0.00234 mol) were added and the mixture was stirred at rt for 1 h. The mixture was quenched with water and extracted twice with EtOAc. The organic layer was dried over MgSO₄, filtered and concentrated under reduced pressure. The residue was taken up in heptane and concentrated under reduced pressure (repeated 3 times) to give the title compound (1.20 g, 96%), which was used without further purification in the next step.

1H NMR (400 MHz, DMSO) δ (ppm): 7.72 (s, 1H), 7.39-7.30 (m, 3H), 7.26-7.22 (m, 2H), 5.21 (s, 2H), 3.71 (s, 3H).

### Int 360.3: 1-benzyl-3-bromo-pyrazol-4-ol

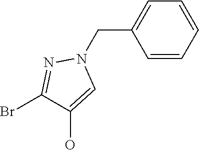 [SMILES: Oc1cn(Cc2ccccc2)nc1Br]

At rt, to a solution of 1-benzyl-3-bromo-4-methoxy-pyrazole (1.20 g, 0.00449 mol) in 12 ml of 1,2-dichloroethane was carefully added AlCl₃ (3.00 g, 0.0225 mol). The mixture was stirred for 1 h, then cooled with an ice bath and carefully neutralized with a 1N aqueous solution of NaOH. The organic layer was separated, dried over MgSO₄, filtered and concentrated under reduced pressure to give the title compound (0.819 g, 72%), which was used without further purification in the next step.

LCMS (Method S): Rt=1.16; MS m/z [M+H]+=253.1/255.1

### Int 360.4: tert-butyl N-[2-(1-benzyl-3-bromo-pyrazol-4-yl)oxyethyl]carbamate

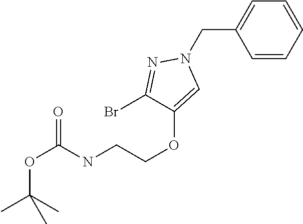 [SMILES: CC(C)(C)OC(=O)NCCOc1cn(Cc2ccccc2)nc1Br]

In a sealed tube, a solution of 1-benzyl-3-bromo-pyrazol-4-ol (815 mg, 0.00322 mol), tert-butyl N-(2-hydroxyethyl)carbamate (0.747 mL, 0.00483 mol) and Cyanomethylenetributylphosphorane (1.69 mL, 0.00644 mol) in 6 ml of toluene was stirred at 100° C. for 1 h. After cooling down to rt, the reaction was concentrated under reduced pressure. The residue was purified by flash chromatography on silica gel using heptane/EtOAc (90/10 to 70/30) to give the title compound as a pale brown oil (0.870 g, 68%).

LCMS (Method S): Rt=1.36; MS m/z [M+H]+=340.1/342.1

### Int 360.5: 2-(1-benzyl-3-bromo-pyrazol-4-yl)oxyethanamine

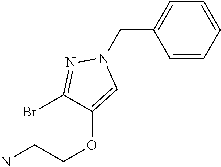 [SMILES: NCCOc1cn(Cc2ccccc2)nc1Br]

TFA (1.00 mL, 0.0135 mol) was added to a solution of tert-butyl N-[2-(1-benzyl-3-bromo-pyrazol-4-yl)oxyethyl]carbamate (0.500 g, 0.00126 mol) in 10 ml of dichloromethane. The solution was stirred at rt for 18 h. The reaction was cooled with an ice bath and slowly basified with a 1N aqueous solution of NaOH until pH about 8-9. The organic layer was separated, dried over MgSO₄, filtered and concentrated under reduced pressure to give the title compound (0.292 g, 78%) as a pale orange oil.

1H NMR (400 MHz, DMSO) δ (ppm): 7.78 (s, 1H), 7.57-7.29 (m, 3H), 7.28-7.19 (m, 2H), 6.36 (bs, 2H), 5.22 (s, 2H), 3.98 (t, J=5.4 Hz, 2H), 3.06 (t, J=5.4 Hz, 2H).

### Int 360.6: 2-benzyl-6,7-dihydro-5H-pyrazolo[4,3-b][1,4]oxazine

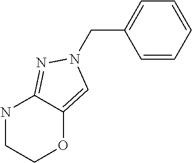 [SMILES: c1ccc(Cn2cc3c(n2)NCCO3)cc1]

In a sealed tube, a solution of 2-(1-benzyl-3-bromo-pyrazol-4-yl)oxyethanamine (0.370 g, 0.00125 mol) in 15 ml of tetrahydrofuran was purged with N₂. GPhos Pd G3 (0.113 g, 0.000125 mol) was added, the mixture was purged again with N₂. NaOTMS 1M in THF (1.00 mol/L, 2.50 mL, 0.00250 mol) was added, the tube was sealed, and the mixture was heated at 80° C. for 18 h. After cooling down to rt, EtOAc and water were added to the reaction. The aqueous layer was separated and extracted twice with EtOAc. The combined organic layers were dried over MgSO₄, filtered off and concentrated under reduced pressure. The residue was purified by flash chromatography on silica gel using DCM/MeOH (100/0 to 95/5) to give the title compound (0.165 g, 61%).

LCMS (Method S): Rt=1.15; MS m/z [M+H]+=216.2

### Int 344.3: tert-butyl (4aR,8aR)-2,3,4,4a,5,7,8,8a-octahydropyrido[4,3-b][1,4]oxazine-6-carboxylate

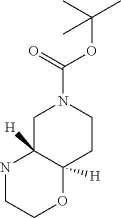 [SMILES: CC(C)(C)OC(=O)N1CC[C@H]2OCCN[C@@H]2C1]

### Int 344.1: tert-butyl (3R,4R)-3-[(2-chloroacetyl)amino]-4-hydroxy-piperidine-1-carboxylate

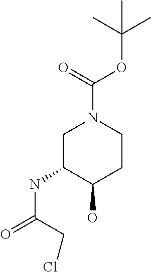 [SMILES: CC(C)(C)OC(=O)N1CC[C@@H](O)[C@H](NC(=O)CCl)C1]

To a stirred solution of tert-butyl (3R,4R)-3-amino-4-hydroxy-piperidine-1-carboxylate (3.00 g, 13.9 mmol) in 8 ml of water and 48 ml of acetone were added sodium acetate (2.5 g, 30.5 mmol) and 2-chloroacetyl chloride (1.33 mL, 16.6 mmol). The reaction mixture was stirred at rt for 18 h. The reaction mixture was concentrated under reduced pressure. Water and DCM were added, the aqueous layer was extracted with DCM. The combined organic layers were washed with water, dried over MgSO₄, filtered and concentrated under reduced pressure to give the title compound (3.72 g, 92%) which was used without further purification in the next step.

¹H NMR (500 MHz, DMSO-d₆) δ (ppm): 8.06 (d, J=7.5 Hz, 1H), 4.92 (d, J=4.8 Hz, 1H), 4.08 (s, 2H), 3.80-3.76 (m, 1H), 3.69-3.66 (m, 1H), 3.44 (m, 2H), 2.92 (t, J=11.7 Hz, 1H), 1.84 (m, 1H), 1.40 (s, 9H), 1.31 (m, 2H).

### Int 344.2: tert-butyl (4aR,8aR)-3-oxo-4,4a,5,7,8,8a-hexahydropyrido[4,3-b][1,4]oxazine-6-carboxylate

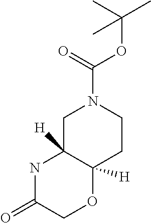 [SMILES: CC(C)(C)OC(=O)N1CC[C@H]2OCC(=O)N[C@@H]2C1]

At 0° C., to a stirred solution of tert-butyl (3R,4R)-3-[(2-chloroacetyl)amino]-4-hydroxy-piperidine-1-carboxylate (3.72 g, 0.0127 mol) in 57 ml of dichloromethane was added dropwise tBuOK (1.43 g mg, 0.0127 mol) in 145 ml of propan-2-ol. The reaction mixture was stirred at rt for 18 h. The reaction mixture was concentrated under reduced pressure, and EtOAc and water were added. The aqueous layer was separated and extracted twice with EtOAc. The combined organic layers were dried over MgSO₄, filtered and concentrated under reduced pressure. The residue was purified by flash chromatography on silica gel using DCM/(MeOH/NH₄OH (10%)) (100/0 to 93/7) to give the title compound as a white solid (2.04 g, 63%).

¹H NMR (500 MHz, DMSO-d₆) δ (ppm): 8.22 (s, 1H), 4.17-4.11 (m, 1H), 4.08 (d, J=3.8 Hz, 2H), 4.05 (s, 1H), 3.37 (m, 1H), 3.01-2.94 (m, 1H), 2.81 (s, 1H), 1.88-1.81 (m, 1H), 1.40 (d, J=3.0 Hz, 9H), 1.38-1.29 (m, 2H).

### Int 344.3: tert-butyl (4aR,8aR)-2,3,4,4a,5,7,8,8a-octahydropyrido[4,3-b][1,4]oxazine-6-carboxylate

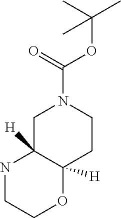 [SMILES: CC(C)(C)OC(=O)N1CC[C@H]2OCCN[C@@H]2C1]

At 0° C., under N₂, to a stirred solution of tert-butyl (4aR,8aR)-3-oxo-4,4a,5,7,8,8a-hexahydropyrido[4,3-b][1,4]oxazine-6-carboxylate (2.53 g, 0.00987 mol) in 55 ml of dichloromethane were added TMSCl (1.50 mL, 0.0118 mol) and LiAlH₄ (1M in THF) (13.8 mL, 0.0138 mol). The reaction mixture was stirred at rt for 18 h. Then, 2 ml of (3M) NaOH solution was slowly added, and the resulting mixture was extracted with DCM. The organic layer was dried over MgSO₄, filtered, and concentrated under reduced pressure to give 2.03 g of the title compound which was used without further purification in the next step.

¹H NMR (400 MHz, DMSO-d₆) δ (ppm): 3.96-3.92 (m, 1H), 3.89-3.85 (m, 1H), 3.73-3.64 (m, 1H), 3.45 (td, J=11.2, 3.1 Hz, 1H), 3.08-2.95 (m, 1H), 2.81-2.61 (m, 3H), 2.48-2.43 (m, 1H), 2.21-2.11 (m, 1H), 1.65 (ddt, J=12.6, 4.8, 2.6 Hz, 1H), 1.38 (s, 9H), 1.34-1.22 (m, 2H).

### Pharmacological Data

### Biological Assays and Data

The activity of a compound according to the present invention can be assessed by the following in vitro methods.

### IL-1β Secretion Assay

Monocytic THP-1 cells were maintained in RPMI 1640 media (Gibco 21875)+10% FBS, (Gibco 10500)+1% penicillin/streptomycin (Gibco 15140-122). Cells were then plated at 40,000 cells per well in 384-well cell culture plates (CoRNning 3542) and maintained with RPMI1640 (Gibco 11835)+5% FBS (Gibco 10500). Activation of the NLRP3 inflammasome requires both an NF-kB-dependent priming step and the addition of a NLRP3 activator. The priming step was induced by LPS (10 ng/mL, InvivoGen ref tlrl-3pelps) for 3 h at 37° C., then compound, in a 1:3 serial dilution series in DMSO (final concentration DMSO 0.1%), and the activator nigericin (Sigma Aldrich, ref: SML1779) 10 μM (final concentration) were added to the cells and co-incubated for 2 hours. 16 μL supernatant was removed, and IL-1β levels were monitored using an HTRF assay (Human IL1b Kits HTRF Cisbio ref: 62HILBPEH) according to manufacturers' instructions.

### TNF-α Secretion Assay

Monocytic THP-1 cells were maintained in RPMI 1640 media (Gibco 21875)+10% FBS, (Gibco 10500)+1% penicillin/streptomycin (Gibco 15140-122). Cells were then plated at 40,000 cells per well in 384-well cell culture plates (Corning 3542) and maintained with RPMI1640 (Gibco 11835)+5% FBS, (Gibco 10500). Experimental compounds were prepared and added as described above. TNF-α secretion was triggered by the addition of 10 ng/mL LPS (invivoGen, tlrl-3pelps) and cells were incubated for 3 hours. TNF-α levels were monitored directly in the cells plate using an Lumit assay (Lumit TNFa-Human Immunoassay—Promega W6051) according to manufacturers' instructions.

### Data Interpretation

Data were expressed as percentage of inhibition (I %) as compared to a maximum signal control containing no small molecule (I %=0%) according to the formula: 1%=(1−((Sample−Min))/((Max−Min)))×100, where:
Sample: Signal obtained in the presence of each compound tested,
Max: Maximum signal in the absence of compound added,
Min: Minimum or background signal obtained in the absence of activation signal.

Relative IC50 (IC50 rel) were obtained from a dose response curve with 10 concentrations. Final IC50 rels are expressed as the geometric mean.

Relative IC50 values were estimated with Biost@t-SPEED v2.4 internal software based on SAS system using the 4-parameter logistic model according to Ratkovsky and Reedy (D. A. Ratkovsky, T. J. Reedy, Choosing near-linear parameters in the four parameters logistic model radioligands and related assays. Biometrics, 42 (1986), 575-582): Y=A+C/((1+exp(−B*(log(X)−M)))), where:
A: Lower asymptote (BOTTOM),
A+C: Upper asymptote (TOP),
M: the logarithm of the concentration estimated at the inflexion point (the logarithm of the relative IC50),
B: Slope at the inflexion point of the curve.

The results of NLRP3 IC50 (that is to say IL-1β IC50) and TNF-α IC50 are summarized in the table below:

| | IL-1β | TNF-α |
|---|---|---|
| Example | IC50 (μM) | IC50 (uM) |
| 1 | 0.407 | >10 |
| 2 | 1.960 | |
| 3 | 2.030 | |
| 4 | 0.251 | >10 |
| 5 | 0.290 | >10 |
| 6 | 0.235 | >10 |
| 7 | 5.470 | |
| 8 | 0.020 | >10 |
| 9 | 0.113 | >10 |
| 10 | 0.178 | >10 |
| 11 | 0.003 | >10 |
| 12 | 0.171 | >10 |
| 13 | 0.002 | >10 |
| 14 | 0.605 | >10 |
| 15 | 0.406 | >10 |
| 16 | 0.025 | >10 |
| 17 | 0.473 | >10 |
| 18 | 0.173 | >10 |
| 19 | 1.480 | |
| 20 | 0.044 | >10 |
| 21 | 0.020 | >10 |
| 22 | 0.153 | >10 |
| 23 | 0.192 | >10 |
| 24 | 0.638 | |
| 25 | 0.109 | >10 |
| 26 | 0.352 | >10 |
| 27 | 1.260 | |
| 28 | 0.109 | >10 |
| 29 | 0.332 | >10 |
| 30 | 0.431 | >10 |
| 31 | 0.520 | >10 |
| 32 | 3.500 | |
| 33 | 0.239 | >10 |
| 34 | 0.095 | >10 |
| 35 | 0.501 | >10 |
| 36 | 0.096 | >10 |
| 37 | 0.893 | |
| 38 | 0.090 | >10 |
| 39 | 0.238 | >10 |
| 40 | 0.420 | >10 |
| 41 | 0.252 | >10 |
| 42 | 0.134 | >10 |
| 43 | 0.603 | |
| 44 | 0.862 | |
| 45 | 0.281 | >10 |
| 46 | 0.389 | >10 |
| 47 | 1.040 | |
| 48 | 0.386 | >10 |
| 49 | 0.672 | |
| 50 | 0.549 | |
| 51 | 1.050 | |
| 52 | 1.250 | |
| 53 | 0.275 | >10 |
| 54 | 0.132 | >10 |
| 55 | 0.176 | >10 |
| 56 | 0.111 | >10 |
| 57 | 0.248 | >10 |
| 58 | 0.064 | >10 |
| 59 | 0.203 | >10 |
| 60 | 0.386 | >10 |
| 61 | 0.090 | >10 |
| 62 | 0.052 | >10 |
| 63 | 0.006 | >10 |
| 64 | 0.222 | >10 |
| 65 | 0.151 | >10 |
| 66 | 0.304 | >10 |
| 67 | 0.306 | >10 |
| 68 | 1.220 | |
| 69 | 0.200 | >10 |
| 70 | 0.467 | >10 |
| 71 | 0.270 | >10 |
| 72 | 0.107 | >10 |
| 73 | 0.056 | >10 |
| 74 | 0.411 | >10 |
| 75 | 0.183 | 9,6 |
| 76 | 0.066 | 8.6 |
| 77 | 0.135 | >10 |
| 78 | 0.436 | >10 |
| 79 | 0.285 | 9.5 |
| 80 | 0.208 | >10 |
| 81 | 0.164 | 9.8 |
| 82 | 0.481 | |
| 83 | 2.791 | |
| 84 | 1.765 | |
| 85 | 0.033 | >10 |
| 86 | 0.506 | >10 |
| 87 | 0.311 | >10 |
| 88 | 1.150 | |
| 89 | 0.614 | |
| 90 | 0.659 | |
| 91 | 4.930 | |
| 92 | 2.200 | |
| 93 | 0.879 | |
| 94 | 1.920 | |
| 95 | 2.200 | |
| 96 | 0.124 | |
| 97 | 0.038 | |
| 98 | 0.298 | |
| 99 | 0.752 | |
| 102 | >10 | |
| 103 | 1.360 | |
| 105 | 0.335 | |
| 106 | 0.174 | |
| 107 | 0.385 | |
| 108 | 0.545 | |
| 109 | 0.089 | |
| 110 | 3.760 | |
| 111 | 0.496 | |
| 112 | 0.088 | |
| 113 | 0.857 | |
| 114 | 0.958 | |
| 115 | 1.540 | |
| 116 | 0.454 | >10 |
| 117 | 0.869 | |
| 118 | 0.758 | |
| 119 | 0.561 | >10 |
| 120 | 2.520 | |
| 121 | 0.092 | >10 |
| 122 | 0.149 | >10 |
| 123 | 2.020 | |
| 124 | 1.050 | |
| 125 | 1.320 | |
| 126 | 0.484 | >10 |
| 127 | 0.125 | >10 |
| 128 | 0.779 | |
| 129 | 0.665 | >10 |
| 130 | 0.099 | >10 |
| 131 | 0.387 | >10 |
| 132 | 0.362 | >10 |
| 133 | 0.068 | >10 |
| 134 | 0.071 | >10 |
| 135 | 0.997 | |
| 136 | 3.030 | |
| 137 | 0.411 | >10 |
| 138 | 0.441 | >10 |
| 139 | 0.235 | >10 |
| 140 | 0.210 | >10 |
| 141 | 0.131 | >10 |
| 142 | 0.360 | >10 |
| 143 | 0.221 | >10 |
| 144 | 0.151 | >10 |
| 145 | 0.034 | >10 |
| 146 | 0.242 | >10 |
| 147 | 0.575 | >10 |
| 148 | 0.922 | |
| 149 | 0.052 | >10 |
| 150 | 1.789 | |
| 151 | 1.170 | |
| 152 | 5.980 | |
| 153 | 0.047 | >10 |
| 154 | 0.344 | >10 |
| 155 | 0.461 | >10 |
| 156 | 1.060 | |
| 157 | 0.314 | >10 |
| 158 | 0.085 | >10 |
| 159 | 0.043 | >10 |
| 160 | 0.171 | >10 |
| 161 | 0.028 | >10 |
| 162 | 0.010 | >10 |
| 163 | 0.007 | >10 |
| 164 | 0.081 | >10 |
| 165 | 0.042 | 5.62 |
| 166 | 3.310 | |
| 167 | 0.309 | >10 |
| 168 | 3.380 | |
| 169 | 0.008 | >10 |
| 170 | 0.012 | >10 |
| 171 | 0.007 | >10 |
| 172 | 0.036 | >10 |
| 173 | 0.040 | >10 |
| 174 | 0.170 | >10 |
| 175 | 0.522 | >10 |
| 176 | 0.250 | >10 |
| 177 | 0.091 | 7.38 |
| 178 | 0.007 | >10 |
| 179 | 0.897 | |
| 180 | 0.473 | >10 |
| 181 | 1.390 | |
| 182 | 1.650 | |
| 183 | 0.685 | |
| 184 | 0.660 | |
| 185 | 3.220 | |
| 186 | 1.050 | |
| 187 | 0.066 | >10 |
| 188 | 0.414 | >10 |
| 189 | 0.012 | >10 |
| 190 | 1.148 | |
| 191 | 1.117 | |
| 192 | 0.011 | >10 |
| 193 | 0.534 | 9,83 |
| 194 | 0.004 | >10 |
| 195 | 0.014 | >10 |
| 196 | 0.452 | >10 |
| 197 | 0.009 | >10 |
| 198 | 0.016 | >10 |
| 199 | 0.004 | >10 |
| 200 | 0.023 | >10 |
| 201 | 0.076 | >10 |
| 203 | 0.332 | 9.99 |
| 204 | 0.054 | >10 |
| 206 | 0.325 | >10 |
| 207 | 0.174 | 3.01 |
| 208 | 0.972 | |
| 209 | 0.002 | >10 |
| 210 | 0.001 | >10 |
| 211 | 0.034 | >10 |
| 212 | 0.039 | >10 |
| 213 | 0.115 | >10 |
| 214 | 0.036 | >10 |
| 215 | 1.460 | |
| 216 | 0.128 | >10 |
| 217 | 0.127 | >10 |
| 219 | 0.103 | >10 |
| 220 | 0.108 | >10 |
| 221 | 0.018 | >10 |
| 222 | 0.042 | >10 |
| 223 | 0.001 | >10 |
| 224 | 0.001 | >10 |
| 225 | 0.003 | >10 |
| 226 | 0.002 | >10 |
| 227 | 1.190 | |
| 228 | 0.271 | >10 |
| 229 | 0.042 | >10 |
| 230 | 0.556 | 9,14 |
| 231 | 0.219 | >10 |
| 232 | 0.502 | 7.63 |
| 233 | 0.164 | >10 |
| 234 | 0.355 | >10 |
| 235 | 0.070 | 7.13 |
| 236 | 0.551 | 9.65 |
| 237 | 0.024 | 4.12 |
| 238 | 0.047 | 4.29 |
| 239 | 0.200 | >10 |
| 240 | 0.421 | >10 |
| 241 | 0.250 | >10 |
| 242 | 2.120 | |
| 243 | 0.021 | >10 |
| 245 | 0.064 | >10 |
| 246 | 0.012 | >10 |
| 247 | 0.168 | >10 |
| 248 | 0.081 | >10 |
| 249 | 0.151 | >10 |
| 250 | 0.249 | >10 |
| 251 | 0.021 | >10 |
| 252 | 0.461 | >10 |
| 253 | 6.750 | |
| 254 | 0.460 | 9,97 |
| 255 | 0.249 | >10 |
| 256 | 0.001 | >10 |
| 257 | 7.490 | |
| 258 | 0.030 | >10 |
| 259 | 0.067 | >10 |
| 260 | 0.180 | >10 |
| 261 | 0.001 | >10 |
| 262 | 0.017 | >10 |
| 263 | 0.180 | >10 |
| 264 | 0.069 | >10 |
| 265 | 0.314 | >10 |
| 266 | 0.073 | >10 |
| 267 | 0.185 | >10 |
| 268 | 0.263 | >10 |
| 269 | 0.404 | >10 |
| 270 | 0.493 | >10 |
| 271 | 0.690 | |
| 272 | 2.34 | 6,58 |
| 273 | 0.456 | >10 |
| 274 | 0.171 | >10 |
| 275 | 0.127 | >10 |
| 276 | 0.515 | >10 |
| 277 | 0.051 | 2,04 |
| 278 | 0.050 | >10 |
| 279 | 0.277 | >10 |
| 280 | 0.076 | >10 |
| 283 | 0.135 | |
| 284 | 0.087 | >10 |
| 285 | 0.065 | >10 |
| 286 | 0.151 | |
| 287 | 0.115 | |
| 288 | 0.143 | |
| 289 | 0.140 | |
| 290 | 0.145 | |
| 291 | 0.001 | >10 |
| 292 | 0.265 | |
| 293 | 0.084 | >10 |
| 295 | 0.002 | >10 |
| 296 | 0.028 | >10 |
| 297 | 0.011 | >10 |
| 298 | 0.003 | >10 |
| 299 | 0.015 | >10 |
| 300 | 0.177 | |
| 301 | 0.868 | |
| 302 | 0.013 | >10 |
| 303 | 3.870 | |
| 304 | 0.142 | |
| 305 | 2.820 | |
| 306 | 1.510 | |
| 307 | 0.410 | |
| 308 | 0.009 | >10 |
| 309 | 0.249 | |
| 310 | 0.496 | |
| 311 | 0.002 | >10 |
| 312 | 0.056 | >10 |
| 313 | 0.0003 | >10 |
| 314 | 0.018 | >10 |
| 315 | 0.511 | |
| 316 | 0.287 | |
| 317 | 3.000 | |
| 318 | 0.883 | |
| 320 | 0.116 | |
| 321 | 0.001 | >10 |
| 322 | 0.080 | >10 |
| 323 | 0.003 | >10 |
| 324 | 0.056 | >10 |
| 325 | 0.275 | |
| 326 | 0.198 | |
| 327 | 0.292 | |
| 328 | 0.219 | |
| 329 | 0.711 | |
| 330 | 0.987 | |
| 331 | 1.080 | |
| 332 | 0.396 | |
| 333 | 0.460 | |
| 334 | 0.073 | |
| 335 | 0.481 | |
| 336 | 1.160 | |
| 337 | 2.880 | |
| 338 | 0.046 | >10 |
| 339 | 0.133 | |
| 340 | 0.0004 | >10 |
| 341 | 0.001 | >10 |
| 342 | 0.002 | >10 |
| 343 | 0.047 | >10 |
| 344 | 0.223 | |
| 345 | 0.125 | |
| 346 | 0.212 | |
| 348 | 0.199 | |
| 349 | 0.953 | |
| 350 | 0.001 | >10 |
| 351 | 0.167 | |
| 352 | 0.375 | |
| 353 | 0.029 | >10 |
| 354 | 0.020 | >10 |
| 355 | 1.170 | |
| 356 | 0.061 | >10 |
| 357 | 0.048 | >10 |
| 358 | 0.011 | >10 |
| 359 | 0.098 | >10 |
| 360 | 0.420 | |
| 361 | 0.008 | >10 |
| 362 | 3.540 | |
| 363 | 0.016 | >10 |
| 364 | 0.858 | |
| 365 | 0.028 | >10 |
| 366 | 0.025 | >10 |
| 367 | 0.0005 | >10 |
| 368 | 0.188 | |
| 369 | 0.476 | |
| 370 | 1.270 | |
| 371 | 0.001 | >10 |
| 372 | 0.094 | >10 |
| 373 | 0.0011 | >10 |
| 374 | 0.042 | >10 |
| 375 | 0.0004 | >10 |
| 376 | 0.180 | |
| 377 | 0.296 | |
| 378 | 0.001 | >10 |
| 379 | 0.001 | >10 |
| 380 | 0.004 | >10 |
| 381 | 0.003 | >10 |
| 382 | 0.012 | >10 |
| 383 | 1.010 | |
| 384 | 0.0004 | >10 |
| 385 | 0.002 | >10 |
| 386 | 0.019 | >10 |
| 387 | 0.031 | >10 |
| 388 | 0.001 | >10 |
| 389 | 0.002 | >10 |
| 390 | 0.033 | >10 |
| 391 | 0.031 | >10 |
| 392 | 0.001 | >10 |
| 393 | 0.461 | |
| 394 | 0.310 | |
| 395 | 0.234 | |
| 397 | 0.023 | >10 |
| 398 | 0.017 | >10 |
| 399 | 0.564 | |
| 400 | 0.093 | >10 |
| 402 | 0.022 | |
| 403 | 0.366 | |
| 404 | 0.00075 | |
| 405 | 0.089 | >10 |
| 406 | 0.078 | >10 |
| 407 | 0.003 | >10 |
| 408 | 0.002 | >10 |
| 409 | 0.638 | |
| 410 | 2.18 | |
| 411 | 0.344 | |
| 412 | 0.018 | >10 |
| 413 | 0.5 | |
| 414 | 0.699 | |
| 415 | 4.66 | |
| 416 | 0.001 | >10 |
| 417 | 0.716 | |
| 418 | 0.013 | >10 |
| 419 | 0.127 | |
| 420 | 0.601 | |
| 421 | 1.23 | |
| 422 | 1.14 | |
| 423 | 0.00048 | >10 |
| 424 | 0.396 | |
| 425 | 0.055 | >10 |
| 426 | 0.952 | |
| 427 | 0.002 | >10 |
| 428 | 1.02 | |
| 429 | <0.00036 | >10 |
| 430 | <0.0003 | >10 |
| 431 | 0.062 | >10 |
| 432 | 0.005 | >10 |
| 433 | 0.002 | >10 |
| 434 | 0.042 | >10 |
| 435 | 0.003 | >10 |
| 436 | 0.002 | >10 |
| 437 | 0.001 | >10 |
| 438 | 0.411 | |
| 439 | 0.569 | |
| 440 | 3.28 | |
| 441 | 4.67 | |
| 442 | 0.195 | |
| 443 | 0.01 | >10 |
| 444 | 4.06 | |
| 445 | 1.900 | |
| 446 | 0.815 | |
| 448 | 0.370 | |
| 449 | 4.190 | |
| 450 | 2.040 | |
| 451 | 1.650 | |
| 452 | 0.005 | |
| 455 | 7.150 | |
| 456 | 0.002 | |
| 457 | 0.432 | |
| 458 | 0.002 | |
| 459 | 0.001 | |
| 460 | <0.0003 | |
| 461 | 0.001 | |
| 462 | 0.251 | |
| 463 | 3.780 | |
| 466 | 0.1 | |
| 467 | 0.014 | |
| 468 | 0.544 | |
| 469 | 0.001 | |
| 470 | 0.00038 | |
| 471 | 0.001 | |
| 472 | 0.001 | |
| 473 | 0.005 | |
| 474 | <0.0003 | |
| 475 | 0.093 | |
| 476 | 0.209 | |
| 477 | <0.0003 | |
| 479 | 0.005 | |
| 480 | 0.006 | |
| 481 | 0.002 | |
| 482 | 0.172 | |
| 483 | 0.001 | |
| 484 | 0.025 | |
| 485 | 0.045 | |
| 486 | <0.0003 | |
| 487 | 0.001 | |
| 488 | 0.912 | |
| 489 | 0.232 | |
| 490 | 0.001 | |
| 491 | 0.185 | |
| 492 | 0.003 | |
| 493 | 0.171 | |
| 494 | 0.001 | |
| 496 | 0.546 | |
| 497 | 0.0004 | |
| 498 | 0.951 | |
| 499 | 0.003 | |
| 500 | 0.062 | |
| 501 | <0.0003 | |
| 502 | 0.347 | |
| 503 | 0.0005 | |
| 504 | 1.08 | |
| 505 | 0.0004 | |
| 506 | 0.135 | |
| 507 | 0.001 | |
| 508 | 0.04 | |
| 509 | 0.016 | |
| 510 | 0.28 | |
| 511 | 0.571 | |
| 512 | 0.276 | |
| 513 | 1.77 | |
| 514 | 0.196 | |
| 515 | 0.044 | |
| 516 | 1.08 | |
| 517 | 1.34 | |
| 518 | 1.22 | |
| 519 | 0.007 | |
| 520 | 0.055 | |
| 521 | 0.028 | |
| 522 | 0.021 | |
| 523 | 0.681 | |
| 524 | 0.008 | |
| 525 | 0.631 | |
| 526 | 0.001 | |
| 527 | 0.020 | |
| 528 | 0.0049 | |

The compounds of the present invention have significant inhibitory activity on secretion of IL-1 beta by THP-1 cells with minimal to no interference with the NF-kB pathway as measured by the TNFalpha secretion after LPS priming.

It is therefore apparent that the compounds of formula (I) have an inhibitory activity on NOD-like receptor protein 3 (NLRP3) inflammasome.

The compounds of formula (I) may thus be used as inhibitors of NOD-like receptor protein 3 (NLRP3) inflammasome pathway.

The compounds of formula (I) may thus be used as medicaments, especially medicaments which are inhibitors of NOD-like receptor protein 3 (NLRP3) inflammasome pathway.

Thus, according to another of its aspects, a subject of the present disclosure is medicaments that comprise a compound of formula (I), or an addition salt thereof with a pharmaceutically acceptable acid.

These medicaments are employed therapeutically in the treatment of neurodegenerative diseases, in particular Parkinson's disease, frontotemporal Dementia, Multiple System Atrophy, Alzheimer's Disease, Multiple Sclerosis, Amyotrophic Lateral Sclerosis or Brain injury.

According to another embodiment, the present disclosure relates to pharmaceutical compositions comprising as active principle, a compound of formula (I).

These pharmaceutical compositions contain an effective dose of at least one compound of formula (I), or a pharmaceutically acceptable salt of the said compound.

These pharmaceutical compositions may also contain at least one pharmaceutically acceptable excipient.

The said excipients are chosen, according to the pharmaceutical form and the desired mode of administration, from the usual excipients known to those skilled in the art.

The compounds of formula (I) may be used in the prevention and/or in the treatment of pathologies involving NOD-like receptor protein 3 (NLRP3) inflammasome pathway.

The present disclosure, according to another of its aspects, also provides a method of treating and/or preventing the pathologies indicated above.

Thus, is also described a method of treating and/or preventing Parkinson's disease, frontotemporal Dementia, Multiple System Atrophy, Alzheimer's Disease, Multiple Sclerosis, Amyotrophic Lateral Sclerosis or Brain injury, including administering to a subject in need thereof a therapeutically effective amount of at least one compound of formula (I) or a pharmaceutically acceptable salt thereof.

According to one embodiment, is also described a method of treating and/or preventing Parkinson's disease, including administering to a subject in need thereof a therapeutically effective amount of at least one compound of formula (I) or a pharmaceutically acceptable salt thereof.

According to another embodiment, is also described a method of treating and/or preventing frontotemporal Dementia, including administering to a subject in need thereof a therapeutically effective amount of at least one compound of formula (I) or a pharmaceutically acceptable salt thereof.

According to another embodiment, is also described a method of treating and/or preventing Multiple System Atrophy, including administering to a subject in need thereof a therapeutically effective amount of at least one compound of formula (I) or a pharmaceutically acceptable salt thereof.

According to another embodiment, is also described a method of treating and/or preventing Alzheimer's Disease, including administering to a subject in need thereof a therapeutically effective amount of at least one compound of formula (I) or a pharmaceutically acceptable salt thereof.

According to another embodiment, is also described a method of treating and/or preventing Multiple Sclerosis, including administering to a subject in need thereof a therapeutically effective amount of at least one compound of formula (I) or a pharmaceutically acceptable salt thereof.

According to another embodiment, is also described a method of treating and/or preventing Amyotrophic Lateral Sclerosis, including administering to a subject in need thereof a therapeutically effective amount of at least one compound of formula (I) or a pharmaceutically acceptable salt thereof.

According to another embodiment, is also described a method of treating and/or preventing Brain injury, including administering to a subject in need thereof a therapeutically effective amount of at least one compound of formula (I) or a pharmaceutically acceptable salt thereof.

Numbered Embodiment 1: A compound of formula (I) or a pharmaceutically acceptable salt thereof:

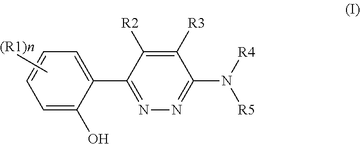

wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

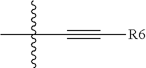

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

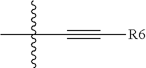

in which R6 is a hydrogen atom or a methyl group and the symbol [symbol] represents the attachment site between the carbon atom and the pyridazine ring;
or R2 and R3 which are on adjacent carbon atoms form together with the atoms connecting them a (C₅-C₆)carbocyclic ring which is fused to the pyridazine ring; and
R4 and R5 form together with the nitrogen atom to which they are attached:
   a 3 to 7 membered monocyclic heterocycloalkyl ring comprising one nitrogen atom and optionally one additional heteroatom selected from a nitrogen atom, an oxygen atom and a sulfur atom and comprising 2 to 6 carbon atoms,
said monocyclic heterocycloalkyl ring being unsubstituted or substituted by one to two substituents independently selected from a (C₁-C₄)alkyl- group, a NH₂ group, a mono(C₁-C₄)alkylamino- group, a di(C₁-C₄)alkylamino- group, a NH₂—(C₁-C₄)alkyl- group, a mono(C₁-C₄)alkylamino-(C₁-C₄)alkyl- group, a di(C₁-C₄)alkylamino-(C₁-C₄)alkyl-group, NH₂—(C₃-C₆)cycloalkyl- group, a OH—(C₃-C₆)cycloalkyl- group, a mono(C₁-C₄)alkylamino-(C₃-C₆)cycloalkyl- group, a di(C₁-C₄)alkylamino-(C₃-C₆)cycloalkyl-group, a (C₂-C₅)acylamino-(C₁-C₄)alkyl- group, a (C₂-C₅)acylamino- group, a (C₂-C₅)acyl group, a hydroxy-(C₁-C₄)alkyl- group, a SH—(C₁-C₄)alkyl- group, a CN—(C₁-C₄)alkyl- group, a (C₁-C₄)alkoxy-(C₁-C₄)alkyl- group, a hydroxyl group, an oxo group, a (C₃-C₆)cycloalkyl group, a halogen atom, a halo(C₁-C₄)alkyl- group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl- group, a mono(C₁-C₄)alkylamino-C(═O)—(C₁-C₄)alkyl- group, a di(C₁-C₄)alkylamino-C(═O)—(C₁-C₄)alkyl- group or a —CO₂H group.

Numbered Embodiment 2: A compound of formula (I) or a pharmaceutically acceptable salt thereof of Embodiment 1, wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

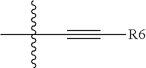

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

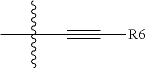

in which R6 is a hydrogen atom or a methyl group and the symbol [symbol] represents the attachment site between the carbon atom and the pyridazine ring; and
R4 and R5 form together with the nitrogen atom to which they are attached:
   a 3 to 7 membered monocyclic heterocycloalkyl ring comprising one nitrogen atom and optionally one additional heteroatom selected from a nitrogen atom, an oxygen atom and a sulfur atom and comprising 2 to 6 carbon atoms,
said monocyclic heterocycloalkyl ring being unsubstituted or substituted by one to two substituents independently selected from a (C₁-C₄)alkyl- group, a NH₂ group, a mono(C₁-C₄)alkylamino- group, a di(C₁-C₄)alkylamino- group, a NH₂—(C₁-C₄)alkyl- group, a mono(C₁-C₄)alkylamino-(C₁-C₄)alkyl- group, a di(C₁-C₄)alkylamino-(C₁-C₄)alkyl- group, NH₂—(C₃-C₆)cycloalkyl- group, a OH—(C₃-C₆)cycloalkyl- group, a mono(C₁-C₄)alkylamino-(C₃-C₆)cycloalkyl- group, a di(C₁-C₄)alkylamino-(C₃-C₆)cycloalkyl- group, a (C₂-C₅)acylamino-(C₁-C₄)alkyl- group, a (C₂-C₅)acylamino- group, a (C₂-C₅)acyl group, a hydroxy-(C₁-C₄)alkyl- group, a SH—(C₁-C₄)alkyl- group, a CN—(C₁-C₄)alkyl- group, a (C₁-C₄)alkoxy-(C₁-C₄)alkyl- group, a hydroxyl group, an oxo group, a (C₃-C₆)cycloalkyl group, a halogen atom, a halo(C₁-C₄)alkyl- group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl- group, a mono(C₁-C₄)alkylamino-C(═O)—(C₁-C₄)alkyl- group, a di(C₁-C₄)alkylamino-C(═O)—(C₁-C₄)alkyl- group or a —CO₂H group.

Numbered Embodiment 3: A compound of formula (I) or a pharmaceutically acceptable salt thereof of Embodiment 1, wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

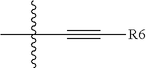

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 which are on adjacent carbon atoms form together with the atoms connecting them a (C₅-C₆)carbocyclic ring which is fused to the pyridazine ring; and
R4 and R5 form together with the nitrogen atom to which they are attached:
   a 3 to 7 membered monocyclic heterocycloalkyl ring comprising one nitrogen atom and optionally one additional heteroatom selected from a nitrogen atom, an oxygen atom and a sulfur atom and comprising 2 to 6 carbon atoms,
said monocyclic heterocycloalkyl ring being unsubstituted or substituted by one to two substituents independently selected from a (C₁-C₄)alkyl- group, a NH₂ group, a mono(C₁-C₄)alkylamino- group, a di(C₁-C₄)alkylamino- group, a NH₂—(C₁-C₄)alkyl- group, a mono(C₁-C₄)alkylamino-(C₁-C₄)alkyl- group, a di(C₁-C₄)alkylamino-(C₁-C₄)alkyl- group, NH₂—(C₃-C₆)cycloalkyl- group, a OH—(C₃-C₆)cycloalkyl- group, a mono(C₁-C₄)alkylamino-(C₃-C₆)cycloalkyl- group, a di(C₁-C₄)alkylamino-(C₃-C₆)cycloalkyl- group, a (C₂-C₅)acylamino-(C₁-C₄)alkyl- group, a (C₂-C₅)acylamino- group, a (C₂-C₅)acyl group, a hydroxy-(C₁-C₄)alkyl- group, a SH—(C₁-C₄)alkyl- group, a CN—(C₁-C₄)alkyl- group, a (C₁-C₄)alkoxy-(C₁-C₄)alkyl- group, a hydroxyl group, an oxo group, a (C₃-C₆)cycloalkyl group, a halogen atom, a halo(C₁-C₄)alkyl- group, a cyano group, a NH₂—C(═O)— group, a mono(C₁-C₄)alkylamino-C(═O)— group, a di(C₁-C₄)alkylamino-C(═O)— group, a NH₂—C(═O)—(C₁—C₄)alkyl- group, a mono(C₁-C₄)alkylamino-C(═O)—(C₁-C₄)alkyl- group, a di(C₁-C₄)alkylamino-C(═O)—(C₁-C₄)alkyl- group or a —CO₂H group.

Numbered Embodiment 4: A compound of formula (II) or a pharmaceutically acceptable salt thereof:

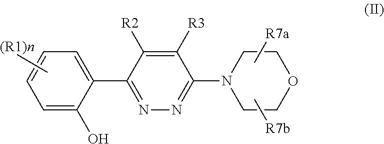

wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

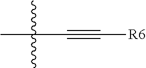

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

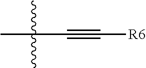

in which R6 is a hydrogen atom or a methyl group and the symbol [symbol] represents the attachment site between the carbon atom and the pyridazine ring;
or R2 and R3 which are on adjacent carbon atoms form together with the atoms 20 connecting them a (C₅-C₆)carbocyclic ring which is fused to the pyridazine ring; and
R7a and R7b represent independently of each other a hydrogen atom, a (C₁-C₄)alkyl- group, a NH₂ group, a mono(C₁-C₄)alkylamino- group, a di(C₁-C₄)alkylamino-group, a NH₂—(C₁-C₄)alkyl- group, a mono(C₁-C₄)alkylamino-(C₁-C₄)alkyl- group, a di(C₁-C₄)alkylamino-(C₁-C₄)alkyl- group, NH₂—(C₃-C₆)cycloalkyl- group, a OH—(C₃-C₆)cycloalkyl- group, a mono(C₁-C₄)alkylamino-(C₃-C₆)cycloalkyl- group, a di(C₁-C₄)alkylamino-(C₃-C₆)cycloalkyl- group, a (C₂-C₅)acylamino-(C₁-C₄)alkyl- group, a (C₂—C₅)acylamino- group, a (C₂-C₅)acyl group, a hydroxy-(C₁-C₄)alkyl- group, a SH—(C₁-C₄)alkyl- group, a CN—(C₁-C₄)alkyl- group, a (C₁-C₄)alkoxy-(C₁-C₄)alkyl- group, a hydroxyl group, an oxo group, a (C₃-C₆)cycloalkyl group, a halogen atom, a halo(C₁-C₄)alkyl- group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl- group, a mono(C₁-C₄)alkylamino-C(═O)—(C₁-C₄)alkyl- group, a di(C₁-C₄)alkylamino-C(═O)—(C₁-C₄)alkyl-group or a —CO₂H group.

Numbered Embodiment 5: A compound of formula (II) or a pharmaceutically acceptable salt thereof of Embodiment 4, wherein
n is 1 or 2;
R1 represent independently of each other a halogen atom, a (C₁-C₄)alkyl group, a (C₁-C₄)alkoxy group, a halo(C₁-C₄)alkoxy group, a halo(C₁-C₄)alkyl group, a group of formula

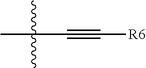

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group or a halo(C₁-C₄)alkyl group;
or R2 and R3 which are on adjacent carbon atoms form together with the atoms connecting them a (C₅-C₆)carbocyclic ring which is fused to the pyridazine ring; and
R7a and R7b represent independently of each other a hydrogen atom, a (C₁-C₄)alkyl- group, a halo-(C₁-C₄)alkyl- group, a NH₂—C(═O)— group, NH₂—(C₁-C₄)alkyl- group, a mono(C₁-C₄)alkylamino-(C₁-C₄)alkyl- group, a di(C₁-C₄)alkylamino-(C₁-C₄)alkyl- group, a (C₂-C₅)acylamino-(C₁-C₄)alkyl- group, a hydroxy-(C₁-C₄)alkyl- group, a SH—(C₁-C₄)alkyl-group, a CN—(C₁-C₄)alkyl- group, a (C₂-C₅)acyl group, a mono(C₁-C₄)alkylamino-C(═O)— group, a di(C₁-C₄)alkylamino-C(═O)— group, a NH₂—(C₃-C₆)cycloalkyl- group, a NH₂—C(═O)—(C₁-C₄)alkyl- group, a mono(C₁-C₄)alkylamino-C(═O)—(C₁-C₄)alkyl- group, a di(C₁-C₄)alkylamino-C(═O)—(C₁-C₄)alkyl- group, an oxo group, or a (C₁-C₄)alkoxy-(C₁-C₄)alkyl-group.

Numbered Embodiment 6: A compound of formula (II) or a pharmaceutically acceptable salt thereof of any of Embodiments 4 or 5, wherein
n is 1 or 2;
R1 represent independently of each other a halogen atom, a (C₁-C₄)alkyl group, a (C₁-C₄)alkoxy group, a halo(C₁-C₄)alkoxy group, a halo(C₁-C₄)alkyl group, a group of formula

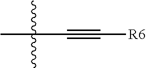

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group or a halo(C₁-C₄)alkyl group;
or R2 and R3 which are on adjacent carbon atoms form together with the atoms connecting them a (C₅-C₆)carbocyclic ring which is fused to the pyridazine ring;
R7a represents a hydrogen atom, a (C₁-C₄)alkyl- group, a halo-(C₁-C₄)alkyl-group, a NH₂—C(═O)— group, NH₂—(C₁-C₄)alkyl- group, a mono(C₁-C₄)alkylamino-(C₁-C₄)alkyl- group, a di(C₁-C₄)alkylamino-(C₁-C₄)alkyl- group, a (C₂-C₅)acylamino-(C₁-C₄)alkyl- group, a hydroxy-(C₁-C₄)alkyl- group, a SH—(C₁-C₄)alkyl- group, a CN—(C₁-C₄)alkyl- group, a (C₂-C₅)acyl group, a mono(C₁-C₄)alkylamino-C(═O)— group, a di(C₁-C₄)alkylamino-C(═O)— group, a NH₂—(C₃-C₆)cycloalkyl- group, a NH₂—C(═O)—(C₁-C₄)alkyl-group, a mono(C₁-C₄)alkylamino-C(═O)—(C₁-C₄)alkyl- group, a di(C₁-C₄)alkylamino-C(═O)—(C₁-C₄)alkyl- group, an oxo group or a (C₁-C₄)alkoxy-(C₁-C₄)alkyl- group; and
R7b represents a hydrogen atom, or a hydroxy-(C₁-C₄)alkyl- group.

Numbered Embodiment 7: A compound of formula (II) or a pharmaceutically acceptable salt thereof of any of Embodiments 4 to 6, wherein
n is 1 or 2
R1 represent independently of each other a methyl group, an ethyl group, an isopropyl group, an ethynyl group, F, Cl, a methoxy group, a cyclopropyl group, a trifluoromethoxy group or a trifluoromethyl group;
or two R1 which are on adjacent carbon atoms form together with the atoms connecting them a cyclopentane ring which is fused to the phenol group so as to form the following formula

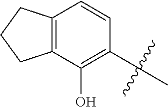 [SMILES: *c1ccc2c(c1O)CCC2]

in which the symbol [symbol] represents the attachment site between the carbon atom of the phenol ring and the carbon atom of the pyridazine ring;
R2 and R3 represent independently of each other a hydrogen atom, a cyano group, a —CHF₂ group, a trifluoromethyl group, a OH—CH₂— group, a methyl group, an ethyl group, or an isopropyl group;
or R2 and R3 which are on adjacent carbon atoms form together with the atoms connecting them a cyclopentyl ring which is fused to the pyridazine ring; and
R7a and R7b represent independently of each other a hydrogen atom, a methyl group, a CH₂F— group, NH₂—CH₂— group, a N(CH₃)₂—CH₂— group, a SH—CH₂— group, a CN—CH₂— group, a CH₃—NH—(CH₂)₂— group, a OH—CH₂— group, a OH-CD2-group, a OH—(CH₂)₂— group, a OH—C(CH₃)₂— group, a CH₃—CH(OH)— group, a CH₃—C(═O)—NH—CH₂— group, a CH₃—C(═O)—NH—CH₂—CH₂— group a CH(CH₃)₂—C(═O)—NH—CH₂— group, a NH₂—C(═O)— group, a NH(CH₃)—C(═O)— group, a N(CH₃)₂—C(═O)— group, a NH₂-cyclopropyl- group, a NH₂—C(═O)—CH₂— group, a NH(CH₃)—C(═O)—CH₂— group, a N(CH₃)₂—C(═O)—CH₂— group, a CH₃—CH₂—C(═O)— group, an oxo group, or a CH₃—O—CH₂— group.

Numbered Embodiment 8: A compound of formula (II) or a pharmaceutically acceptable salt thereof of any of Embodiments 4 to 7, wherein
n is 1 or 2;
R1 represent independently of each other a methyl group, an ethyl group, an isopropyl group, an ethynyl group, F, Cl, a methoxy group, a cyclopropyl group, a trifluoromethoxy group or a trifluoromethyl group;
or two R1 which are on adjacent carbon atoms form together with the atoms connecting them a cyclopentane ring which is fused to the phenol group so as to form the following formula

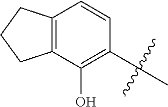 [SMILES: *c1ccc2c(c1O)CCC2]

in which the symbol [symbol] represents the attachment site between the carbon atom of the phenol ring and the carbon atom of the pyridazine ring;
R2 and R3 represent independently of each other a hydrogen atom, a cyano group, a —CHF₂ group, a trifluoromethyl group, a OH—CH₂— group, a methyl group, an ethyl group, or an isopropyl group;
or R2 and R3 which are on adjacent carbon atoms form together with the atoms connecting them a cyclopentyl ring which is fused to the pyridazine ring;
R7a represents a hydrogen atom, a methyl group, a CH₂F— group, NH₂—CH₂— group, a N(CH₃)₂—CH₂— group, a SH—CH₂— group, a CN—CH₂— group, a CH₃—NH—(CH₂)₂— group, a OH—CH₂— group, a OH-CD2-group, a OH—(CH₂)₂— group, a OH—C(CH₃)₂— group, a CH₃—CH(OH)— group, a CH₃—C(═O)—NH—CH₂— group, a CH₃—C(═O)—NH—CH₂—CH₂— group a CH(CH₃)₂—C(═O)—NH—CH₂— group, a NH₂—C(═O)— group, a NH(CH₃)—C(═O)— group, a N(CH₃)₂—C(═O)— group, a NH₂-cyclopropyl- group, a NH₂—C(═O)—CH₂— group, a NH(CH₃)—C(═O)—CH₂— group, a N(CH₃)₂—C(═O)—CH₂— group, a CH₃—CH₂—C(═O)— group, an oxo group, or a CH₃—O—CH₂— group; and
R7b represents a hydrogen atom or a OH—CH₂— group.

Numbered Embodiment 9: A compound of formula (III) or a pharmaceutically acceptable salt thereof:

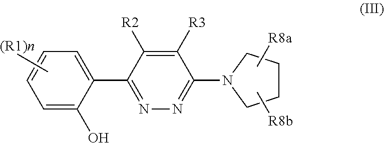

wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

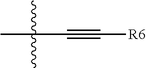

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

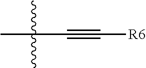

in which R6 is a hydrogen atom or a methyl group and the symbol [symbol] represents the attachment site between the carbon atom and the pyridazine ring;
or R2 and R3 which are on adjacent carbon atoms form together with the atoms connecting them a (C₅-C₆)carbocyclic ring which is fused to the pyridazine ring; and
R8a and R8b represent independently of each other a hydrogen atom, a (C₁-C₄)alkyl- group, a NH₂ group, a mono(C₁-C₄)alkylamino- group, a di(C₁-C₄)alkylamino-group, a NH₂—(C₁-C₄)alkyl- group, a mono(C₁-C₄)alkylamino-(C₁-C₄)alkyl- group, a di(C₁-C₄)alkylamino-(C₁-C₄)alkyl- group, NH₂—(C₃-C₆)cycloalkyl- group, a OH—(C₃-C₆)cycloalkyl- group, a mono(C₁-C₄)alkylamino-(C₃-C₆)cycloalkyl- group, a di(C₁-C₄)alkylamino-(C₃-C₆)cycloalkyl- group, a (C₂-C₅)acylamino-(C₁-C₄)alkyl- group, a (C₂-C₅)acylamino- group, a (C₂-C₅)acyl group, a hydroxy-(C₁-C₄)alkyl- group, a SH—(C₁-C₄)alkyl- group, a CN—(C₁-C₄)alkyl- group, a (C₁-C₄)alkoxy-(C₁-C₄)alkyl- group, a hydroxyl group, an oxo group, a (C₃-C₆)cycloalkyl group, a halogen atom, a halo(C₁-C₄)alkyl- group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl- group, a mono(C₁-C₄)alkylamino-C(═O)—(C₁-C₄)alkyl- group, a di(C₁-C₄)alkylamino-C(═O)—(C₁-C₄)alkyl-group or a —CO₂H group.

Numbered Embodiment 10: A compound of formula (III) or a pharmaceutically acceptable salt thereof of Embodiment 9, wherein
n is 2;
R1 represent independently of each other a halogen atom, a (C₁-C₄)alkyl group, or a halo(C₁-C₄)alkyl group;
R2 and R3 represent a hydrogen atom, or a halo(C₁-C₄)alkyl group; and
R8a and R8b represent independently of each other a hydrogen atom, a NH₂— group, a NH₂—(C₁-C₄)alkyl- group, a di(C₁-C₄)alkylamino-(C₁-C₄)alkyl- group, a hydroxy-(C₁-C₄)alkyl- group, a (C₁-C₄)alkoxy-(C₁-C₄)alkyl- group, a hydroxyl group, or an oxo group.

Numbered Embodiment 11: A compound of formula (III) or a pharmaceutically acceptable salt thereof of any of Embodiments 9 or 10, wherein
n is 2;
R1 represent independently of each other a halogen atom, a (C₁-C₄)alkyl group, or a halo(C₁-C₄)alkyl group;
R2 represents a halo(C₁-C₄)alkyl group or a hydrogen atom;
R3 represents a hydrogen atom;
R8a represents a hydrogen atom, a NH₂— group, a NH₂—(C₁-C₄)alkyl- group, a di(C₁-C₄)alkylamino-(C₁-C₄)alkyl- group, a hydroxy-(C₁-C₄)alkyl- group, a (C₁-C₄)alkoxy-(C₁-C₄)alkyl- group, a hydroxyl group, or an oxo group; and
R8b represents a hydrogen atom, or an oxo group.

Numbered Embodiment 12: A compound of formula (III) or a pharmaceutically acceptable salt thereof of any of Embodiments 9 to 11, wherein
n is 2;
R1 represent independently of each other a chlorine atom, a methyl group, or a trifluoromethyl group;
R2 represents a hydrogen atom or a —CHF₂ group;
R3 represents a hydrogen atom; and
R8a and R8b represent independently of each other a hydrogen atom, a NH₂— group, a NH₂—(CH₂)— group, a N(CH₃)₂—(CH₂)— group, a OH—CH₂— group, a CH₃—O—CH₂— group, a hydroxyl group, or an oxo group.

Numbered Embodiment 13: A compound of formula (III) or a pharmaceutically acceptable salt thereof of any of Embodiments 9 to 12, wherein
n is 2;
R1 represent independently of each other a chlorine atom, a methyl group, or a trifluoromethyl group;
R2 represents a hydrogen atom or a —CHF₂ group;
R3 represents a hydrogen atom;
R8a represents a hydrogen atom, a NH₂— group, a NH₂—(CH₂)— group, a N(CH₃)₂—(CH₂)— group, a OH—CH₂— group, a CH₃—O—CH₂— group, a hydroxyl group, or an oxo group; and
R8b represents a hydrogen atom, or an oxo group.

Numbered Embodiment 14: A compound of formula (IV) or a pharmaceutically acceptable salt thereof:

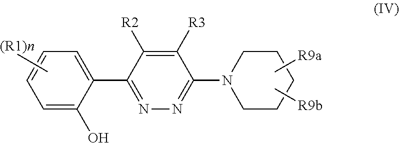

wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula

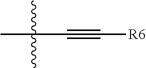

in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula

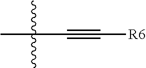

in which R6 is a hydrogen atom or a methyl group and the symbol [symbol] represents the attachment site between the carbon atom and the pyridazine ring;
or R2 and R3 which are on adjacent carbon atoms form together with the atoms connecting them a (C₅-C₆)carbocyclic ring which is fused to the pyridazine ring;
R9a and R9b represent independently of each other a hydrogen atom, a (C₁-C₄)alkyl- group, a NH₂ group, a mono(C₁-C₄)alkylamino- group, a di(C₁-C₄)alkylamino-group, a NH₂—(C₁-C₄)alkyl- group, a mono(C₁-C₄)alkylamino-(C₁-C₄)alkyl- group, a di(C₁-C₄)alkylamino-(C₁-C₄)alkyl- group, NH₂—(C₃-C₆)cycloalkyl- group, a OH—(C₃-C₆)cycloalkyl- group, a mono(C₁-C₄)alkylamino-(C₃-C₆)cycloalkyl- group, a di(C₁-C₄)alkylamino-(C₃-C₆)cycloalkyl- group, a (C₂-C₅)acylamino-(C₁-C₄)alkyl- group, a (C₂-C₅)acylamino- group, a (C₂-C₅)acyl group, a hydroxy-(C₁-C₄)alkyl- group, a SH—(C₁-C₄)alkyl- group, a CN—(C₁-C₄)alkyl- group, a (C₁-C₄)alkoxy-(C₁-C₄)alkyl- group, a hydroxyl group, an oxo group, a (C₃-C₆)cycloalkyl group, a halogen atom, a halo(C₁-C₄)alkyl- group, a cyano group, a NH₂—C(═O)— group, a mono(C₁-C₄)alkylamino-C(═O)— group, a di(C₁—C₄)alkylamino-C(═O)— group, a NH₂—C(═O)—(C₁-C₄)alkyl- group, a mono(C₁-C₄)alkylamino-C(═O)—(C₁-C₄)alkyl- group, a di(C₁-C₄)alkylamino-C(═O)—(C₁-C₄)alkyl-group or a —CO₂H group.

Numbered Embodiment 15: A compound of formula (IV) or a pharmaceutically acceptable salt thereof of Embodiment 14, wherein
n is 2;
R1 represent independently of each other a halogen atom, a (C₁-C₄)alkyl group, or a halo(C₁-C₄)alkyl group;
R2 and R3 represent a hydrogen atom; and
R9a and R9b represent independently of each other a hydrogen atom, a NH₂ group, a mono(C₁-C₄)alkylamino- group, a di(C₁-C₄)alkylamino- group, a NH₂—(C₁-C₄)alkyl- group, a di(C₁-C₄)alkylamino-(C₁-C₄)alkyl- group, NH₂—(C₃-C₆)cycloalkyl- group, a (C₂-C₅)acylamino-(C₁-C₄)alkyl- group, a hydroxy-(C₁-C₄)alkyl- group, a hydroxyl group, a cyano group, an oxo group or a halogen atom.

Numbered Embodiment 16: A compound of formula (IV) or a pharmaceutically acceptable salt thereof of any of Embodiments 14 or 15, wherein
n is 2;
R1 represent independently of each other a halogen atom, a (C₁-C₄)alkyl group, or a halo(C₁-C₄)alkyl group;
R2 and R3 represent a hydrogen atom;
R9a represents a hydrogen atom, a NH₂ group, a mono(C₁-C₄)alkylamino-group, a di(C₁-C₄)alkylamino- group, a NH₂—(C₁-C₄)alkyl- group, a di(C₁-C₄)alkylamino-(C₁-C₄)alkyl- group, NH₂—(C₃-C₆)cycloalkyl- group, a (C₂-C₅)acylamino-(C₁-C₄)alkyl-group, a hydroxy-(C₁-C₄)alkyl- group, a hydroxyl group, a cyano group, or a halogen atom; and
R9b represents a hydrogen atom, an oxo group, or a halogen atom.

Numbered Embodiment 17: A compound of formula (IV) or a pharmaceutically acceptable salt thereof of any of Embodiments 14 to 16, wherein
n is 2;
R1 represent independently of each other a chlorine atom, a methyl group, or a trifluoromethyl group;
R2 and R3 represent a hydrogen atom; and
R9a and R9b represent independently of each other a hydrogen atom, a NH₂ group, a N(CH₃)₂— group, a CH₃—CH₂—NH— group, a NH₂—CH₂— group, a N(CH₃)₂—CH₂— group, a NH₂-cyclopropyl- group, a CH₃—(C=O)—NH—CH₂— group, a OH—CH₂— group, a hydroxyl group, a cyano group, an oxo group or a fluorine atom (F).

Numbered Embodiment 18. A compound of formula (IV) or a pharmaceutically acceptable salt thereof of any of Embodiments 14 to 17, wherein
n is 2;
R1 represent independently of each other a chlorine atom, a methyl group, or a trifluoromethyl group;
R2 and R3 represent a hydrogen atom;
R9a represents a hydrogen atom, a NH₂ group, a N(CH₃)₂— group, a CH₃—CH₂—NH— group, a NH₂—CH₂— group, a N(CH₃)₂—CH₂— group, a NH₂-cyclopropyl- group, a CH₃—(C=O)—NH—CH₂— group, a OH—CH₂— group, a hydroxyl group, a cyano group, or a fluorine atom (F); and
R9b represents a hydrogen atom, an oxo group, or a fluorine atom (F).

1: A compound of formula (I) or a pharmaceutically acceptable salt thereof 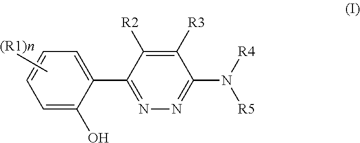
wherein
n is 1 or 2;
R1 represent independently of each other a halogen atom, a (C₁-C₄)alkyl group, a halo(C₁-C₄)alkyl group, a (C₁-C₄)alkoxy group, a halo(C₁-C₄)alkoxy group, a group of formula 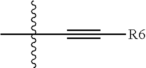
in which R6 is a hydrogen atom or a methyl group and the symbol [symbol] represents the attachment site between the carbon atom and the phenyl ring, or a (C₃-C₆)cycloalkyl group;
or two R1 which are on adjacent carbon atoms form together with the atoms connecting them a cyclopentane ring which is fused to the phenol group;
R2 and R3 represent independently of each other a hydrogen atom, a cyano group, a (C₁-C₄)alkyl group, a hydroxy(C₁-C₄)alkyl group, a halo(C₁-C₄)alkyl group, or a group of formula 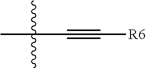
in which R6 is a hydrogen atom or a methyl group and the symbol [symbol] represents the attachment site between the carbon atom and the pyridazine ring;
or R2 and R3 which are on adjacent carbon atoms form together with the atoms connecting them a (C₅-C₆)carbocyclic ring which is fused to the pyridazine ring;
R4 and R5 form together with the nitrogen atom to which they are attached:
a 3 to 7 membered monocyclic heterocycloalkyl ring comprising one nitrogen atom and optionally one additional heteroatom selected from the group consisting of a nitrogen atom, an oxygen atom and a sulfur atom and comprising 2 to 6 carbon atoms,
a 8 to 11 membered bicyclic heterocycloalkyl ring comprising one nitrogen atom and optionally one to three additional heteroatoms independently selected from the group consisting of a nitrogen atom, an oxygen atom and a sulfur atom and comprising 4 to 10 carbon atoms, or
a 7 to 12 membered bicyclic heterocyclic spiro ring comprising one nitrogen atom and optionally one to three additional heteroatoms selected from the group consisting of a nitrogen atom, an oxygen atom, and a sulfur atom and comprising 3 to 11 carbon atoms, said monocyclic heterocycloalkyl ring, bicyclic heterocycloalkyl ring and bicyclic heterocyclic spiro ring being unsubstituted or substituted by one to two substituents independently selected from the group consisting of a (C₁-C₄)alkyl- group, a NH₂ group, a mono(C₁-C₄)alkylamino-group, a di(C₁-C₄)alkylamino- group, a NH₂—(C₁-C₄)alkyl- group, a mono(C₁-C₄)alkylamino-(C₁-C₄)alkyl- group, a di(C₁-C₄)alkylamino-(C₁-C₄)alkyl- group, NH₂—(C₃-C₆)cycloalkyl- group, a OH—(C₃-C₆)cycloalkyl- group, a mono(C₁-C₄)alkylamino-(C₃-C₆)cycloalkyl- group, a di(C₁-C₄)alkylamino-(C₃-C₆)cycloalkyl- group, a (C₂-C₅)acylamino-(C₁-C₄)alkyl- group, a (C₂-C₅)acylamino- group, a (C₂-C₅)acyl group, a hydroxy-(C₁-C₄)alkyl- group, a SH—(C₁-C₄)alkyl-group, a CN—(C₁-C₄)alkyl- group, a (C₁-C₄)alkoxy-(C₁-C₄)alkyl- group, a hydroxyl group, an oxo group, a (C₃-C₆)cycloalkyl group, a halogen atom, a halo(C₁-C₄)alkyl- group, a cyano group, a NH₂—C(═O)— group, a mono(C₁-C₄)alkylamino-C(═O)— group, a di(C₁-C₄)alkylamino-C(═O)— group, a NH₂—C(═O)—(C₁-C₄)alkyl- group, a mono(C₁-C₄)alkylamino-C(═O)—(C₁-C₄)alkyl- group, a di(C₁-C₄)alkylamino-C(═O)—(C₁-C₄)alkyl- group, and a —CO₂H group.

2: The compound of claim 1, or a pharmaceutically acceptable salt thereof, wherein n is 1.

3: The compound of claim 1, or a pharmaceutically acceptable salt thereof, wherein n is 2.

4: The compound of claim 1, or a pharmaceutically acceptable salt thereof, wherein R1 represent independently of each other a fluorine atom, a chlorine atom, a methyl group, an ethyl group, an isopropyl group, a methoxy group, a trifluoromethoxy group, a cyclopropyl group, a —CHF₂ group, an ethynyl group or a trifluoromethyl group, or two R1 which are on adjacent carbon atoms form together with the atoms connecting them a cyclopentane ring which is fused to the phenol group to provide 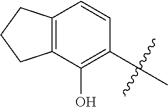 [SMILES: *c1ccc2c(c1O)CCC2]
in which the symbol [symbol] represents the attachment site between the carbon atom of the phenol ring and the carbon atom of the pyridazine ring.

5: The compound of claim 1, or a pharmaceutically acceptable salt thereof, wherein
when n is 1, R1 is in ortho, meta or para position on the phenyl with respect to pyridazine ring, or
when n is 2, the two R1 are in ortho and in para position on the phenyl with respect to pyridazine ring, or
when n is 2, the two R1 are in ortho and in meta position on the phenyl with respect to pyridazine ring.

6: The compound of claim 1, or a pharmaceutically acceptable salt thereof, wherein
R2 and R3 represent independently of each other
a hydrogen atom, a cyano group, a —CHF₂ group, a trifluoromethyl group, a —CH₂OH group, a methyl group, an ethyl group or an isopropyl group.

7: The compound of claim 1, or a pharmaceutically acceptable salt thereof, wherein
R2 and R3 which are on adjacent carbon atoms form together with the atoms connecting them a cyclopentyl ring which is fused to the pyridazine ring.

8: The compound of claim 1, or a pharmaceutically acceptable salt thereof, wherein
R4 and R5 form together with the nitrogen atom to which they are attached:
an azepanyl ring unsubstituted or substituted by one OH—CH₂— group;
a morpholino ring unsubstituted or substituted by one or two substituents independently selected from the group consisting of a methyl group, a CH₂F— group, NH₂—CH₂— group, a N(CH₃)₂—CH₂— group, a CH₃—NH—(CH₂)₂— group, a SH—CH₂— group, a CN—CH₂— group, a OH—CH₂— group, a OH-CD2-group, a OH—(CH₂)₂— group, a OH—C(CH₃)₂— group, a CH₃—CH(OH)— group, a CH₃—C(═O)—NH—CH₂— group, a CH₃—C(═O)—NH—CH₂— CH₂— group a CH(CH₃)₂—C(═O)—NH—CH₂— group, a NH₂—C(═O)— group, a NH(CH₃)—C(═O)— group, a N(CH₃)₂—C(═O)— group, a NH₂-cyclopropyl- group, a NH₂—C(═O)—CH₂— group, a NH(CH₃)—C(═O)—CH₂— group, a N(CH₃)₂—C(═O)—CH₂— group, a CH₃—CH₂—C(═O)— group, an oxo group, and a CH₃—O—CH₂— group;
a thiomorpholino ring unsubstituted or substituted by one to two oxo groups;
a piperidinyl ring unsubstituted or substituted by one to two substituents independently selected from the group consisting of a NH₂ group, a NH₂—CH₂— group, a NH(CH₂CH₃)— group, a N(CH₃)₂— group, a NH₂-cyclopropyl- group, a N(CH₃)₂—CH₂— group, a OH—CH₂— group, a CH₃—C(═O)—NH—CH₂— group, a cyano group, a hydroxyl group, an oxo group and a fluorine atom;
a pyrrolidinyl ring unsubstituted or substituted by one to two substituents independently selected from the group consisting of a NH₂— group, a NH₂—CH₂— group, a N(CH₃)₂—CH₂— group, a OH—CH₂— group, a CH₃—O—CH₂— group, a hydroxyl group, and an oxo group;
an isoxazolidinyl ring unsubstituted or substituted by one hydroxyl group;
a piperazinyl ring unsubstituted or substituted by one to two substituents independently selected from the group consisting of a methyl group, an oxo group and a OH—CH₂— group;
a hexahydropyrimidinyl ring substituted by one oxo group;
an azetidinyl ring unsubstituted or substituted by one OH—CH₂— group;
a tetrahydroquinolinyl ring or a dihydroquinolinyl ring such as 3,4-dihydro-2H-quinolinyl ring, unsubstituted or substituted by one hydroxyl group,
a hexahydropyridooxazinyl ring such as a hexahydro-2H-pyrido[3,4-b][1,4]oxazinyl ring, for instance a 3,4a,5,7,8,8a-hexahydro-2H-pyrido[3,4-b][1,4]oxazinyl group, or a hexahydro-2H-pyrido[4,3-b][1,4]oxazin-4-yl ring, for instance a 3,4a,5,7,8,8a-hexahydro-2H-pyrido[4,3-b][1,4]oxazinyl ring, unsubstituted or substituted by one cyclobutyl group, one methyl group, one ethyl group, one 2,2,2-trifluoroethyl group, one 2-fluoroethyl group, one 3-fluoropropyl group, one isopropropyl group or one acetyl group;
a hexahydropyranooxazinyl ring such as hexahydro-2H-pyrano[3,4-b][1,4]oxazinyl ring, for instance a 3,4a,5,7,8,8a-hexahydro-2H-pyrano[3,4-b][1,4]oxazinyl group, or such as a hexahydro-2H-pyrano[4,3-b][1,4]oxazinyl ring, for instance a 3,4a,5,7,8,8a-hexahydro-2H-pyrano[4,3-b][1,4]oxazinyl ring, unsubstituted;
a dihydropyridooxazinyl ring such as dihydropyrido[3,4-b][1,4]oxazinyl ring or dihydropyrido[4,3-b][1,4]oxazinyl ring, for instance 2,3-dihydropyrido[3,4-b][1,4]oxazinyl ring or 2,3-dihydropyrido[4,3-b][1,4]oxazinyl ring, unsubstituted;
a dihydropyrazolooxazinyl ring such as dihydro-2H-pyrazolo[4,3-b][1,4]oxazinyl ring, for instance 5,6-dihydro-2H-pyrazolo[4,3-b][1,4]oxazinyl ring, unsubstituted;
a dihydrobenzoxazinyl ring such as dihydro-1,4-benzoxazinyl ring, for instance 2,3-dihydro-1,4-benzoxazinyl ring substituted by one hydroxyl group;
a tetrahydronaphthyridinyl ring or a dihydronaphthyridinyl ring such as 3,4-dihydro-2H-1,6-naphthyridinyl ring substituted by one hydroxyl group;
a hexahydronaphthyridinyl ring such as hexahydro-2H-1,7-naphthyridinyl ring for instance a 3,4,4a,5,6,8a-hexahydro-2H-1,7-naphthyridinyl ring substituted by one methyl group and one oxo group;
a octahydronaphthyridinyl ring such as octahydro-1,7-naphthyridinyl ring or octahydro-1,5-naphthyridinyl ring for instance a 2,3,4,4a,5,6,7,8a-octahydro-1,7-naphthyridinyl ring, a 2,3,4,4a,5,6,8,8a-octahydro-1,7-naphthyridinyl ring, or a 2,3,4,4a,6,7,8,8a-octahydro-1,5-naphthyridinyl ring, substituted by one oxo group, one methyl group, or one acetyl group;
an octahydropyrrolopyridinyl ring or a hexahydropyrrolopyridinyl ring such as hexahydro-1H-pyrrolo[3,4-c]pyridinyl ring, octahydropyrrolo[2,3-c]pyridinyl ring, or hexahydro-2H-pyrrolo[2,3-c]pyridinyl ring, for instance 2,3,3a,6,7,7a-hexahydro-1H-pyrrolo[3,4-c]pyridinyl ring, 2,3,3a,4,5,6,7,7a-octahydropyrrolo[2,3-c]pyridinyl ring or 3,3a,4,5,7,7a-hexahydro-2H-pyrrolo[2,3-c]pyridinyl ring, unsubstituted or substituted by one methyl group, one ethyl group, one acetyl group, one isopropyl group or one oxo group;
an octahydropyrrolooxazinyl ring or a hexahydropyrrolooxazinyl ring such as hexahydro-2H-pyrrolo[3,4-b][1,4]oxazinyl ring, for instance 2,3,4a,5,7,7a-hexahydro-2H-pyrrolo[3,4-b][1,4]oxazinyl ring, 3,4,4a,5,7,7a-hexahydro-2H-pyrrolo[3,4-b][1,4]oxazinyl ring or 3,4a,5,6,7,7a-hexahydro-2H-pyrrolo[3,4-b][1,4]oxazinyl ring, unsubstituted or substituted by one methyl group, one ethyl group or one acetyl group;
a hexahydrocyclopentaoxazinyl ring such as 3,4a,5,6,7,7a-hexahydro-2H-cyclopenta[b][1,4]oxazinyl ring substituted by one hydroxyl group;
an octahydropyridooxazinyl ring such as octahydropyrido[4,3-b][1,4]oxazinyl ring, for instance 2,3,4a,5,6,7,8,8a-octahydropyrido[4,3-b][1,4]oxazinyl ring, unsubstituted;
an indolinyl ring substituted by one hydroxyl group or one —CH₂OH group;
a hexahydropyrrolopyrrolyl ring such as hexahydropyrrolo[3,4-b]pyrrolyl ring, hexahydro-1H-pyrrolo[3,2-b]pyrrolyl ring, or hexahydro-2H-pyrrolo[3,4-b]pyrrolyl ring, for instance 2,3,3a,4,6,6a-hexahydropyrrolo[3,4-b]pyrrolyl ring, 2,3,3a,5,6,6a-hexahydro-1H-pyrrolo[3,2-b]pyrrolyl ring, or a 3,3a,4,5,6,6a-hexahydro-2H-pyrrolo[3,4-b]pyrrolyl ring unsubstituted or substituted by one methyl group, one ethyl group or one acetyl group;
a hexahydrofuropyrrolyl ring such as hexahydrofuro[3,4-c]pyrrolyl ring for instance 1,3,3a,4,6,6a-hexahydrofuro[3,4-c]pyrrolyl ring, unsubstituted;
a azabicyclooctanyl ring such as 6-azabicyclo[3.2.1]octanyl ring substituted by one hydroxyl group;
a diazabicyclononanyl ring such as 3,6-diazabicyclo[3.2.2]nonanyl ring substituted by one methyl group;
a oxaazabicyclooctanyl ring such as 3-oxa-8-azabicyclo[3.2.1]octanyl ring unsubstituted;
an azaspirononanyl ring such as an azaspiro[3.5]nonanyl ring, unsubstituted or substituted by one substituent selected from the group consisting of a NH₂ group, a CH₃—C(═O)—NH— group, and a hydroxyl group;
an azaspirooctanyl ring such as an azaspiro[3.4]octanyl ring, unsubstituted or substituted by one hydroxyl group;
an azaspiroheptanyl ring such as an azaspiro[3.3]heptanyl ring, unsubstituted or substituted by one hydroxyl group;
a diazaspirodecanyl ring such as 1,9-diazaspiro[4.5]decanyl ring substituted by one methyl group;
a diazaspirononanyl ring such as a diazaspiro[3.5]nonanyl ring, unsubstituted or substituted by one methyl group;
a diazaspiroheptanyl ring such as a diazaspiro[3.3]heptanyl ring, unsubstituted or substituted by one a methyl group;
an oxadiazaspirononanyl ring such as 8-oxa-2,5-diazaspiro[3.5]nonanyl ring substituted by one methyl group;
an oxadiazaspirodecanyl ring such as 2-oxa-6,9-diazaspiro[4.5]decanyl ring, 9-oxa-2,6-diazaspiro[4.5]decanyl ring, or 6-oxa-2,9-diazaspiro[4.5]decanyl ring unsubstituted or substituted by one ethyl group or one methyl group;
an oxadiazaspiroundecanyl ring such as 4-oxa-1,8-diazaspiro[5.5]undecanyl ring substituted by one methyl group;
an dioxadiazaspirododecanyl ring such as 1,11-dioxa-4,8-diazaspiro[5.6]dodecanyl ring substituted by one ethyl group;
an oxaazaspirononanyl ring such as a oxaazaspiro[3.5]nonanyl ring; or
an oxaazaspiroheptanyl ring such as an oxaazaspiro[3.3]heptanyl ring.

9: The compound of claim 1, or a pharmaceutically acceptable salt thereof, wherein the compound is selected from the group consisting of:
(1) 1-[6-(2-hydroxy-4,6-dimethyl-phenyl)pyridazin-3-yl]piperidin-4-ol;
(2) 1-[6-(2-hydroxy-4,6-dimethyl-phenyl)pyridazin-3-yl]piperidine-4-carbonitrile;
(3) 7-[6-(2-hydroxy-4,6-dimethyl-phenyl)pyridazin-3-yl]-7-azaspiro[3.5]nonan-2-ol;
(4) 3,5-dimethyl-2-[6-[rac-(4aS,8aR)-6-cyclobutyl-3,4a,5,7,8,8a-hexahydro-2H-pyrido[3,4-b][1,4]oxazin-1-yl]pyridazin-3-yl]phenol;
(5) 2-[6-[(3R)-3-(hydroxymethyl)morpholin-4-yl]pyridazin-3-yl]-3,5-dimethyl-phenol;
(6) 2-[6-[(3S)-3-(hydroxymethyl)morpholin-4-yl]pyridazin-3-yl]-3,5-dimethyl-phenol;
(7) 2-[6-(2,7-diazaspiro[3.5]nonan-7-yl)pyridazin-3-yl]-3,5-dimethyl-phenol;2,2,2-trifluoroacetic acid;
(8) 2-[6-[(2S)-2-(hydroxymethyl)morpholin-4-yl]pyridazin-3-yl]-3,5-dimethyl-phenol;
(9) 2-[6-[(2R)-2-(hydroxymethyl)morpholin-4-yl]pyridazin-3-yl]-3,5-dimethyl-phenol;
(10) 2-[6-[(4aR,8aS)-6-cyclobutyl-3,4a,5,7,8,8a-hexahydro-2H-pyrido[4,3-b][1,4] oxazin-4-yl]pyridazin-3-yl]-3,5-dimethyl-phenol Enantiomer 1;
(11) 2-[6-[(4aS,8aR)-6-cyclobutyl-3,4a,5,7,8,8a-hexahydro-2H-pyrido[4,3-b][1,4] oxazin-4-yl]pyridazin-3-yl]-3,5-dimethyl-phenol; formic acid Enantiomer 2;
(12) 2-[6-[(4aR,8aS)-6-cyclobutyl-3,4a,5,7,8,8a-hexahydro-2H-pyrido[4,3-b][1,4] oxazin-4-yl]pyridazin-3-yl]-3-methyl-5-(trifluoromethyl)phenol Enantiomer 1;
(13) 2-[6-[(4aS,8aR)-6-cyclobutyl-3,4a,5,7,8,8a-hexahydro-2H-pyrido[4,3-b][1,4] oxazin-4-yl]pyridazin-3-yl]-3-methyl-5-(trifluoromethyl)phenol Enantiomer 2;
(14) 2-[6-[(2R)-2-(methoxymethyl)pyrrolidin-1-yl]pyridazin-3-yl]-3-methyl-5-(trifluoromethyl)phenol;
(15) 2-[6-[(2S)-2-(methoxymethyl)pyrrolidin-1-yl]pyridazin-3-yl]-3-methyl-5-(trifluoromethyl)phenol;
(16) 2-[6-[(2S)-2-(hydroxymethyl)morpholin-4-yl]pyridazin-3-yl]-3-methyl-5-(trifluoromethyl)phenol;
(17) 2-[6-[3-(1-aminocyclopropyl)-1-piperidyl]pyridazin-3-yl]-3,5-dichloro-phenol;
(18) 2-[6-[(3S)-3-(aminomethyl)-1-piperidyl]pyridazin-3-yl]-3,5-dichloro-phenol;
(19) 2-[6-[4-(aminomethyl)-1-piperidyl]pyridazin-3-yl]-3,5-dichloro-phenol;
(20) 2-[6-[(3S)-3-(hydroxymethyl)pyrrolidin-1-yl]pyridazin-3-yl]-3-methyl-5-(trifluoromethyl)phenol;
(21) 3,5-dichloro-2-[6-[(2S)-2-(hydroxymethyl)morpholin-4-yl]pyridazin-3-yl]phenol;
(22) 2-[6-[(3R)-3-(aminomethyl)-1-piperidyl]pyridazin-3-yl]-3,5-dichloro-phenol;
(23) 2-[6-[(3S)-3-(aminomethyl)pyrrolidin-1-yl]pyridazin-3-yl]-3,5-dichloro-phenol;
(24) 2-[6-[2-[(dimethylamino)methyl]morpholin-4-yl]pyridazin-3-yl]-3-methyl-5-(trifluoromethyl)phenol;
(25) 2-[6-[(3R)-3-(aminomethyl)pyrrolidin-1-yl]pyridazin-3-yl]-3,5-dichloro-phenol;
(26) 2-[6-[2-(1-hydroxy-1-methyl-ethyl)morpholin-4-yl]pyridazin-3-yl]-3-methyl-5-(trifluoromethyl)phenol;
(27) 2-[6-[(2R)-2-[(dimethylamino)methyl]morpholin-4-yl]pyridazin-3-yl]-3-methyl-5-(trifluoromethyl)phenol;
(28) 2-[6-[(3S)-3-(hydroxymethyl)-1-piperidyl]pyridazin-3-yl]-3-methyl-5-(trifluoromethyl)phenol;
(29) 3-methyl-2-[6-(4-methylpiperazin-1-yl)pyridazin-3-yl]-5-(trifluoromethyl) phenol;
(30) 2-[6-[3-(hydroxymethyl)azetidin-1-yl]pyridazin-3-yl]-3-methyl-5-(trifluoromethyl)phenol;
(31) 2-[6-[(3R)-3-amino-1-piperidyl]pyridazin-3-yl]-3,5-dimethyl-phenol; formic acid;
(32) 2-[6-(2-amino-7-azaspiro[3.5]nonan-7-yl)pyridazin-3-yl]-3,5-dichloro-phenol;
(33) 3,5-dichloro-2-[6-[(2R)-2-(methoxymethyl)morpholin-4-yl]pyridazin-3-yl]phenol;
(34) 3,5-dichloro-2-[6-[(3S)-3-(hydroxymethyl)-1-piperidyl]pyridazin-3-yl]phenol;
(35) 3,5-dichloro-2-[6-[(2S)-2-(methoxymethyl)morpholin-4-yl]pyridazin-3-yl]phenol;
(36) 3,5-dichloro-2-[6-[(3R)-3-(hydroxymethyl)-1-piperidyl]pyridazin-3-yl]phenol;
(37) 7-[6-(2,4-dichloro-6-hydroxy-phenyl)pyridazin-3-yl]-7-azaspiro[3.5]nonan-2-ol;
(38) 3,5-dichloro-2-(6-morpholinopyridazin-3-yl)phenol;
(39) 3,5-dichloro-2-[6-[(2S)-2-[(dimethylamino)methyl]morpholin-4-yl]pyridazin-3-yl]phenol;
(40) 3,5-dichloro-2-[6-[(3S)-3-[(dimethylamino)methyl]pyrrolidin-1-yl]pyridazine-3-yl]phenol;
(41) 3,5-dichloro-2-[6-[(3R)-3-[(dimethylamino)methyl]pyrrolidin-1-yl]pyridazin-3-yl]phenol;
(42) 6-[6-(2,4-dichloro-6-hydroxy-phenyl)pyridazin-3-yl]-6-azaspiro[3.4]octan-2-ol;
(43) 3,5-dichloro-2-[6-[(2R)-2-[(dimethylamino)methyl]morpholin-4-yl]pyridazin-3-yl]phenol;
(44) 3-fluoro-2-[6-[(2S)-2-(hydroxymethyl)morpholin-4-yl]pyridazin-3-yl]phenol;
(45) 3-fluoro-2-[6-[(2S)-2-(hydroxymethyl)morpholin-4-yl]pyridazin-3-yl]-5-methyl-phenol;
(46) 2-[6-(2,4-dichloro-6-hydroxy-phenyl)pyridazin-3-yl]-2-azaspiro[3.3]heptan-6-ol;
(47) N-[7-[6-(2,4-dichloro-6-hydroxy-phenyl)pyridazin-3-yl]-7-azaspiro[3.5]nonan-2-yl]acetamide;
(48) 3-chloro-2-[6-[(2S)-2-(hydroxymethyl)morpholin-4-yl]pyridazin-3-yl]phenol;
(49) 3,5-dichloro-2-[6-(4,4-difluoro-1-piperidyl)pyridazin-3-yl]phenol;
(50) 3,5-dichloro-2-[6-[(3R)-3-[(dimethylamino)methyl]-1-piperidyl]pyridazin-3-yl]phenol;
(51) 3,5-dichloro-2-[6-[(3S)-3-[(dimethylamino)methyl]-1-piperidyl]pyridazin-3-yl]phenol;
(52) 2-[6-[2-(1-hydroxy-1-methyl-ethyl)morpholin-4-yl]pyridazin-3-yl]-3-methyl-5-(trifluoromethyl)phenol Enantiomer 1;
(53) 2-[6-[-2-(1-hydroxy-1-methyl-ethyl)morpholin-4-yl]pyridazin-3-yl]-3-methyl-5-(trifluoromethyl)phenol Enantiomer 2;
(54) 2-[6-[(2S)-2-(hydroxymethyl)morpholin-4-yl]pyridazin-3-yl]-3-(trifluoromethyl)phenol;
(55) 3,5-dichloro-2-[6-(2-methyl-2,7-diazaspiro[3.5]nonan-7-yl)pyridazin-3-yl]phenol;
(56) 2-[6-[(2S)-2-(hydroxymethyl)morpholin-4-yl]pyridazin-3-yl]-3-methyl-phenol;
(57) N-[[(3R)-1-[6-(2,4-dichloro-6-hydroxy-phenyl)pyridazin-3-yl]-3-piperidyl]methyl]acetamide;
(58) N-[[(3S)-1-[6-(2,4-dichloro-6-hydroxy-phenyl)pyridazin-3-yl]-3-piperidyl]methyl]acetamide;
(59) 2-[6-[-2-[(dimethylamino)methyl]morpholin-4-yl]pyridazin-3-yl]-3-methyl-5-(trifluoromethyl)phenol Enantiomer 1;
(60) 2-[6-[-2-[(dimethylamino)methyl]morpholin-4-yl]pyridazin-3-yl]-3-methyl-5-(trifluoromethyl)phenol Enantiomer 2;
(61) 1-[6-[2-hydroxy-6-methyl-4-(trifluoromethyl)phenyl]pyridazin-3-yl]pyrrolidin-2-one;
(62) (4S)-4-hydroxy-1-[6-[2-hydroxy-6-methyl-4-(trifluoromethyl)phenyl]pyridazin-3-yl]pyrrolidin-2-one;
(63) 5-chloro-2-[6-[(2S)-2-(hydroxymethyl)morpholin-4-yl]pyridazin-3-yl]-3-methyl-phenol;
(64) 3,5-dichloro-2-[6-(2-oxa-6-azaspiro[3.3]heptan-6-yl)pyridazin-3-yl]phenol;
(65) 2-[6-[(3R)-3-(hydroxymethyl)pyrrolidin-1-yl]pyridazin-3-yl]-3,5-dimethyl-phenol;
(66) 3,5-dichloro-2-[6-(2-oxa-7-azaspiro[3.5]nonan-7-yl)pyridazin-3-yl]phenol;
(67) (3S)-1-[6-(2-hydroxy-4,6-dimethyl-phenyl)pyridazin-3-yl]piperidin-3-ol;
(68) 2-[6-[(3S)-3-(ethylamino)-1-piperidyl]pyridazin-3-yl]-3,5-dimethyl-phenol;
(69) 2-[6-[(3S)-3-amino-1-piperidyl]pyridazin-3-yl]-3,5-dimethyl-phenol;
(70) (3R)-1-[6-(2-hydroxy-4,6-dimethyl-phenyl)pyridazin-3-yl]pyrrolidin-3-ol;
(71) (3S)-1-[6-(2-hydroxy-4,6-dimethyl-phenyl)pyridazin-3-yl]pyrrolidin-3-ol;
(72) 2-[4-ethyl-6-[(2S)-2-(hydroxymethyl)morpholin-4-yl]pyridazin-3-yl]-5-methyl-phenol;
(73) 5-chloro-2-[4-ethyl-6-[(2S)-2-(hydroxymethyl)morpholin-4-yl]pyridazin-3-yl]phenol;
(74) 2-[6-[(2S)-2-(hydroxymethyl)morpholin-4-yl]-4-isopropyl-pyridazin-3-yl]-5-methyl-phenol;
(75) 5-chloro-2-[6-[(2S)-2-(hydroxymethyl)morpholin-4-yl]-4-isopropyl-pyridazin-3-yl]phenol;
(76) 2-[6-[(2S)-2-(hydroxymethyl)morpholin-4-yl]-4-methyl-pyridazin-3-yl]-5-methyl-phenol;
(77) 2-[4-[(2S)-2-(hydroxymethyl)morpholin-4-yl]-6,7-dihydro-5H-cyclopenta[d]pyridazin-1-yl]-5-methyl-phenol;
(78) 5-chloro-2-[4-[(2S)-2-(hydroxymethyl)morpholin-4-yl]-6,7-dihydro-5H-cyclopenta[d]pyridazin-1-yl]phenol;
(79) 2-[6-[(3S)-3-(hydroxymethyl)pyrrolidin-1-yl]pyridazin-3-yl]-3,5-dimethyl-phenol;
(80) 2-[6-[(2S)-2-(hydroxymethyl)morpholin-4-yl]-4,5-dimethyl-pyridazin-3-yl]-5-methyl-phenol;
(81) 5-chloro-2-[6-[(2S)-2-(hydroxymethyl)morpholin-4-yl]-4,5-dimethyl-pyridazin-3-yl]phenol;
(82) 2-[6-[(3S)-3-(methoxymethyl)pyrrolidin-1-yl]pyridazin-3-yl]-3,5-dimethyl-phenol;
(83) 2-[6-(2-hydroxy-4,6-dimethyl-phenyl)pyridazin-3-yl]-2-azaspiro[3.5]nonan-7-ol;
(84) 2-[6-(2-hydroxy-4,6-dimethyl-phenyl)pyridazin-3-yl]-2-azaspiro[3.4]octan-6-ol;
(85) 5-chloro-2-[6-[(2S)-2-(hydroxymethyl)morpholin-4-yl]-4-methyl-pyridazin-3-yl]phenol;
(86) (4S)-4-hydroxy-1-[6-(2-hydroxy-4,6-dimethyl-phenyl)pyridazin-3-yl]pyrrolidin-2-one;
(87) 2-[6-[(2R)-2-(aminomethyl)morpholin-4-yl]pyridazin-3-yl]-3,5-dimethyl-phenol;2,2,2-trifluoroacetic acid;
(88) 2-[6-[(3R)-3-(methoxymethyl)pyrrolidin-1-yl]pyridazin-3-yl]-3,5-dimethyl-phenol;
(89) 2-[6-[(2R)-2-(aminomethyl)morpholin-4-yl]pyridazin-3-yl]-3,5-dimethyl-phenol;2,2,2-trifluoroacetic acid;
(90) 6-[(2S)-2-(hydroxymethyl)morpholin-4-yl]-3-(2-hydroxy-4-methyl-phenyl)pyridazine-4-carbonitrile;
(91) 3,5-dimethyl-2-[6-(1-methyl-1,6-diazaspiro[3.3]heptan-6-yl)pyridazin-3-yl]phenol;
(92) 2-[6-[(2S)-2-(hydroxymethyl)morpholin-4-yl]pyridazin-3-yl]-5-methyl-phenol;
(93) 5-fluoro-2-[6-[(2S)-2-(hydroxymethyl)morpholin-4-yl]-4-methyl-pyridazin-3-yl]phenol;
(94) 2-[6-(2-hydroxy-4,6-dimethyl-phenyl)pyridazin-3-yl]-2-azaspiro[3.4]octan-6-ol Enantiomer 1;
(95) 2-[6-(2-hydroxy-4,6-dimethyl-phenyl)pyridazin-3-yl]-2-azaspiro[3.4]octan-6-ol Enantiomer 2;
(96) 2-[6-[(2S)-2-(hydroxymethyl)morpholin-4-yl]pyridazin-3-yl]-5-methoxy-3-methyl-phenol;
(97) 2-[6-[(2S)-2-(hydroxymethyl)morpholin-4-yl]-4-methyl-pyridazin-3-yl]-5-(trifluoromethyl)phenol;
(98) 2-[6-[2-[(1S)-1-hydroxyethyl]morpholin-4-yl]pyridazin-3-yl]-3,5-dimethylphenol Diastereoisomer 2;
(99) 2-[6-[2-[(1S)-1-hydroxyethyl]morpholin-4-yl]pyridazin-3-yl]-3,5-dimethylphenol Diastereoisomer 1;
(102) 2-[6-[(2S)-2-(hydroxymethyl)morpholin-4-yl]pyridazin-3-yl]-4-methyl-phenol;
(103) 3-(4-chloro-2-hydroxy-phenyl)-6-[(2S)-2-(hydroxymethyl)morpholin-4-yl]pyridazine-4-carbonitrile;
(105) N-[[(2S)-4-[6-(2-hydroxy-4,6-dimethyl-phenyl)pyridazin-3-yl]morpholin-2-yl]methyl]acetamide;
(106) N-[[(2R)-4-[6-(2-hydroxy-4,6-dimethyl-phenyl)pyridazin-3-yl]morpholin-2-yl]methyl]acetamide;
(107) 2-[6-[3-(hydroxymethyl)azepan-1-yl]pyridazin-3-yl]-3,5-dimethyl-phenol;
(108) 2-[6-[2-[(1R)-1-hydroxyethyl]morpholin-4-yl]pyridazin-3-yl]-3,5-dimethyl-phenol Diastereoisomer 1;
(109) 2-[6-[(2S)-2-(hydroxymethyl)morpholin-4-yl]-5-methyl-pyridazin-3-yl]-3,5-dimethyl-phenol;
(110) 2-[6-[(2S)-2-(hydroxymethyl)morpholin-4-yl]-5-methyl-pyridazin-3-yl]-5-methyl-phenol;
(111) 2-[6-[rac-(4aS,8aS)-3,4a,5,7,8,8a-hexahydro-2H-pyrano[3,4-b][1,4]oxazin-1-yl]pyridazin-3-yl]-3,5-dimethyl-phenol;
(112) 2-[6-[(2S)-2-(hydroxymethyl)morpholin-4-yl]-4-methyl-pyridazin-3-yl]-3,5-dimethyl-phenol;
(113) (4R)-2-[6-(2-hydroxy-4,6-dimethyl-phenyl)pyridazin-3-yl]isoxazolidin-4-ol
(114) 2-[6-[1-(hydroxymethyl)-6-azabicyclo[3.2.1]octan-6-yl]pyridazin-3-yl]-3,5-dimethyl-phenol;
(115) 2-[6-[(2S)-2-(hydroxymethyl)azetidin-1-yl]pyridazin-3-yl]-3,5-dimethyl-phenol;
(116) 2-[6-[(2S)-2-(hydroxymethyl)morpholin-4-yl]-4-methyl-pyridazin-3-yl]-5-methoxy-phenol;
(117) 3,5-dichloro-2-[6-[(2S)-2-(hydroxymethyl)azetidin-1-yl]pyridazin-3-yl]phenol;
(118) 2-[6-[(2R)-2-(hydroxymethyl)azetidin-1-yl]pyridazin-3-yl]-3,5-dimethyl-phenol;
(119) 2-[6-[(2S)-2-(hydroxymethyl)morpholin-4-yl]-4-methyl-pyridazin-3-yl]-5-methoxy-3-methyl-phenol;
(120) 3,5-dimethyl-2-[6-(3-oxa-8-azabicyclo[3.2.1]octan-8-yl)pyridazin-3-yl]phenol;
(121) 2-[6-[(2S)-2-(hydroxymethyl)morpholin-4-yl]pyridazin-3-yl]-3-methyl-5-(trifluoromethoxy)phenol;
(122) 1-[6-(2-hydroxy-4,6-dimethyl-phenyl)pyridazin-3-yl]-3-(hydroxymethyl) pyrrolidin-2-one;
(123) 2-[6-[(3R)-3-(aminomethyl)morpholin-4-yl]pyridazin-3-yl]-3,5-dimethyl-phenol, 2,2,2-trifluoroacetic acid;
(124) 2-[6-[(3S)-3-(aminomethyl)morpholin-4-yl]pyridazin-3-yl]-3,5-dimethyl-phenol;
(125) 2-[6-[(4aR,7aR)-3,4,4a,5,7,7a-hexahydro-2H-pyrrolo[3,4-b][1,4]oxazin-6-yl]pyridazin-3-yl]-3,5-dimethylphenol;
(126) 1-[4-[6-(2-hydroxy-4,6-dimethylphenyl)pyridazin-3-yl]morpholin-2-yl]propan-1-one;
(127) 5-chloro-2-[6-[(3S)-3-(hydroxymethyl)pyrrolidin-1-yl]pyridazin-3-yl]-3-methyl-phenol;
(128) 2-[6-[(3S)-3-(dimethylamino)-1-piperidyl]pyridazin-3-yl]-3,5-dimethyl-phenol;
(129) N-[[(3R)-4-[6-(2-hydroxy-4,6-dimethyl-phenyl)pyridazin-3-yl]morpholin-3-yl]methyl]acetamide;
(130) (4S)-1-[6-(4-chloro-2-hydroxy-6-methyl-phenyl)pyridazin-3-yl]-4-hydroxy-pyrrolidin-2-one;
(131) (4S)-4-amino-1-[6-(2-hydroxy-4,6-dimethyl-phenyl)pyridazin-3-yl]pyrrolidin-2-one;
(132) (4R)-4-hydroxy-1-[6-[2-hydroxy-6-methyl-4-(trifluoromethyl)phenyl]pyridazin-3-yl]pyrrolidin-2-one;
(133) 5-chloro-2-[6-[(2S)-2-(hydroxymethyl)morpholin-4-yl]-5-methyl-pyridazin-3-yl]-3-methyl-phenol;
(134) 5-(difluoromethyl)-2-[6-[(2S)-2-(hydroxymethyl)morpholin-4-yl]pyridazin-3-yl]-3-methyl-phenol;
(135) 2-[6-[(2S)-2-(1-aminocyclopropyl)morpholin-4-yl]pyridazin-3-yl]-3,5-dimethyl-phenol;
(136)₂-[6-[(2R)-2-(1-aminocyclopropyl)morpholin-4-yl]pyridazin-3-yl]-3,5-dimethyl-phenol;
(137) (4R)-1-[6-(2-hydroxy-4,6-dimethyl-phenyl)pyridazin-3-yl]-4-(hydroxymethyl) pyrrolidine-2-one;
(138) (4S)-1-[6-(2-hydroxy-4,6-dimethyl-phenyl)pyridazin-3-yl]-4-(hydroxymethyl) pyrrolidin-2-one;
(139) (3R)-1-[6-(2-hydroxy-4,6-dimethyl-phenyl)pyridazin-3-yl]-3-(hydroxymethyl)pyrrolidin-2-one;
(140) (3S)-1-[6-(2-hydroxy-4,6-dimethyl-phenyl)pyridazin-3-yl]-3-(hydroxymethyl)pyrrolidin-2-one;
(141) 2-[6-[(2S)-2-(hydroxymethyl)morpholin-4-yl]pyridazin-3-yl]-3,6-dimethylphenol;
(142) 2-[(2R)-4-[6-(2-hydroxy-4,6-dimethylphenyl)pyridazin-3-yl]morpholin-2-yl]-N-methylacetamide;
(143) 2-[(2S)-4-[6-(2-hydroxy-4,6-dimethylphenyl)pyridazin-3-yl]morpholin-2-yl]-N-methylacetamide;
(144) 2-[4-(difluoromethyl)-6-[(2S)-2-(hydroxymethyl)morpholin-4-yl]pyridazin-3-yl]-5-methyl-phenol;
(145) 5-chloro-2-[4-(difluoromethyl)-6-[(2S)-2-(hydroxymethyl)morpholin-4-yl] pyridazin-3-yl]phenol;
(146) 2-[(2R)-4-[6-(2-hydroxy-4,6-dimethylphenyl)pyridazin-3-yl]morpholin-2-yl] acetamide;
(147) 2-[(2S)-4-[6-(2-hydroxy-4,6-dimethylphenyl)pyridazin-3-yl]morpholin-2-yl] acetamide;
(148) 2-[6-[(3R)-3-(dimethylamino)-1-piperidyl]pyridazin-3-yl]-3,5-dimethyl-phenol;
(149) 5-chloro-2-[6-[(2S)-2-(hydroxymethyl)morpholin-4-yl]-4-methyl-pyridazin-3-yl]-3-methyl-phenol;
(150) 2-[(2R)-4-[6-(2-hydroxy-4,6-dimethylphenyl)pyridazin-3-yl]morpholin-2-yl]-N,N-dimethylacetamide;
(151) 2-[(2S)-4-[6-(2-hydroxy-4,6-dimethylphenyl)pyridazin-3-yl]morpholin-2-yl]-N,N-dimethylacetamide;
(152) 2-[6-[(2S)-2-(hydroxymethyl)morpholin-4-yl]pyridazin-3-yl]-4,6-dimethylphenol;
(153) 3-(difluoromethyl)-2-[6-[(2S)-2-(hydroxymethyl)morpholin-4-yl]pyridazin-3-yl]-5-methyl-phenol;
(154) 3,5-dimethyl-2-[6-[rac-(4aR,8aS)-3,4a,5,7,8,8a-hexahydro-2H-pyrano[3,4-b][1,4]oxazin-1-yl]pyridazin-3-yl]phenol;
(155) 2-[6-[(2S)-2-(hydroxymethyl)morpholin-4-yl]-4-(trifluoromethyl)pyridazin-3-yl]-5-methyl-phenol;
(156) 1-[(2R)-4-[6-(2-hydroxy-4,6-dimethylphenyl)pyridazin-3-yl]morpholin-2-yl] propan-1-one;
(157) 1-[(2S)-4-[6-(2-hydroxy-4,6-dimethylphenyl)pyridazin-3-yl]morpholin-2-yl] propan-1-one;
(158) 6-(2-hydroxy-4,6-dimethyl-phenyl)-3-[(2S)-2-(hydroxymethyl)morpholin-4-yl]pyridazine-4-carbonitrile;
(159) 3-[(2S)-2-(hydroxymethyl)morpholin-4-yl]-6-[2-hydroxy-6-methyl-4-(trifluoromethyl)phenyl]pyridazine-4-carbonitrile;
(160) 5-chloro-2-[6-[(2S)-2-(hydroxymethyl)morpholin-4-yl]-4-(trifluoromethyl) pyridazine-3-yl]phenol;
(161) 3,5-dichloro-2-[6-[2-(fluoromethyl)morpholin-4-yl]pyridazin-3-yl]phenol;
(162) 4-[6-(2-hydroxy-4,6-dimethyl-phenyl)pyridazin-3-yl]-2,3-dihydro-1,4-benzoxazin-6-ol;
(163) 6-(4-chloro-2-hydroxy-6-methyl-phenyl)-3-[(2S)-2-(hydroxymethyl)morpholin-4-yl]pyridazine-4-carbonitrile;
(164) N-[[(3S)-1-[6-[2-hydroxy-6-methyl-4-(trifluoromethyl)phenyl]pyridazin-3-yl]-3-piperidyl]methyl]acetamide;
(165) 2-[6-[(2S)-2-[dideuterio(hydroxy)methyl]morpholin-4-yl]pyridazin-3-yl]-3,5-dimethylphenol;
(166) (2S)-4-[6-(2-hydroxy-4,6-dimethylphenyl)pyridazin-3-yl]-N-methylmorpholine-2-carboxamide;
(167) N-[[(3R)-1-[6-[2-hydroxy-6-methyl-4-(trifluoromethyl)phenyl]pyridazin-3-yl]-3-piperidyl]methyl]acetamide;
(168) 2-[6-[(4aR,7aR)-4-ethyl-2,3,4a,5,7,7a-hexahydropyrrolo[3,4-b][1,4]oxazin-6-yl]pyridazin-3-yl]-3,5-dimethylphenol;
(169) 5-chloro-2-[6-[(2S)-2-[dideuterio(hydroxy)methyl]morpholin-4-yl]pyridazin-3-yl]-3-methylphenol;
(170) 2-[5-(difluoromethyl)-6-[(2S)-2-(hydroxymethyl)morpholin-4-yl]pyridazin-3-yl]-3,5-dimethyl-phenol;
(171) 5-chloro-2-[5-(difluoromethyl)-6-[(2S)-2-(hydroxymethyl)morpholin-4-yl]pyridazin-3-yl]-3-methyl-phenol;
(172) 3-chloro-2-[6-[(2S)-2-(hydroxymethyl)morpholin-4-yl]pyridazin-3-yl]-5-methyl-phenol;
(173) 2-[6-[(2R,6S)-2,6-bis(hydroxymethyl)morpholin-4-yl]pyridazin-3-yl]-3,5-dimethyl-phenol;
(174) 2-[6-[2-(fluoromethyl)morpholin-4-yl]pyridazin-3-yl]-3,5-dimethyl-phenol;
(175) 6-[6-(2-hydroxy-4,6-dimethyl-phenyl)pyridazin-3-yl]-6-azaspiro[3.5]nonan-2-ol Isomer 1;
(176) 6-[6-(2-hydroxy-4,6-dimethyl-phenyl)pyridazin-3-yl]-6-azaspiro[3.5]nonan-2-ol Isomer 2;
(177) 3-chloro-2-[6-[(2S)-2-(hydroxymethyl)morpholin-4-yl]-4-methyl-pyridazin-3-yl]-5-methyl-phenol;
(178) 2-[6-[(2R,6S)-2,6-bis(hydroxymethyl)morpholin-4-yl]pyridazin-3-yl]-3-methyl-5-(trifluoromethyl)phenol;
(179) (4S)-2-[6-(2-hydroxy-4,6-dimethyl-phenyl)pyridazin-3-yl]isoxazolidin-4-ol;
(180) 2-[6-[2-[(1R)-1-hydroxyethyl]morpholin-4-yl]pyridazin-3-yl]-3,5-dimethylphenol Diastereoisomer 2;
(181) (2S)-4-[6-(2-hydroxy-4,6-dimethylphenyl)pyridazin-3-yl]morpholine-2-carboxamide;
(182) (2R)-4-[6-(2-hydroxy-4,6-dimethylphenyl)pyridazin-3-yl]-N-methylmorpholine-2-carboxamide;
(183) (2R)-4-[6-(2-hydroxy-4,6-dimethylphenyl)pyridazin-3-yl]morpholine-2-carboxamide;
(184) (2R)-4-[6-(2-hydroxy-4,6-dimethylphenyl)pyridazin-3-yl]-N,N-dimethylmorpholine-2-carboxamide;
(185) 2-[6-[(4aS,7aS)-3,4,4a,5,7,7a-hexahydro-2H-pyrrolo[3,4-b][1,4]oxazin-6-yl]pyridazin-3-yl]-3,5-dimethylphenol;
(186) 2-[4-(hydroxymethyl)-6-[(2S)-2-(hydroxymethyl)morpholin-4-yl]pyridazin-3-yl]-3,5-dimethyl-phenol;
(187) 2-[5-(hydroxymethyl)-6-[(2S)-2-(hydroxymethyl)morpholin-4-yl]pyridazin-3-yl]-3,5-dimethyl-phenol;
(188) 5-chloro-2-[4-(hydroxymethyl)-6-[(2S)-2-(hydroxymethyl)morpholin-4-yl]pyridazin-3-yl]-3-methyl-phenol;
(189) 5-chloro-2-[5-(hydroxymethyl)-6-[(2S)-2-(hydroxymethyl)morpholin-4-yl]pyridazin-3-yl]-3-methyl-phenol;
(190) 2-[6-[rac-(4aR,7aR)-3,4a,5,6,7,7a-hexahydro-2H-pyrrolo[3,4-b][1,4]oxazin-4-yl]pyridazin-3-yl]-3,5-dimethylphenol;
(191) 2-[6-[(2S)-2-(hydroxymethyl)morpholin-4-yl]pyridazin-3-yl]-3,4-dimethylphenol;
(192) 2-[6-[(2S)-2-[dideuterio(hydroxy)methyl]morpholin-4-yl]pyridazin-3-yl]-3-methyl-5-(trifluoromethyl)phenol;
(193) 4-[6-(4-chloro-2-hydroxy-6-methyl-phenyl)pyridazin-3-yl]-2,3-dihydro-1,4-benzoxazin-7-ol;
(194) 2-[5-(difluoromethyl)-6-[(2S)-2-(hydroxymethyl)morpholin-4-yl]pyridazin-3-yl]-3-methyl-5-(trifluoromethyl)phenol;
(195) 2-[6-[rac-(4aR,8aS)-2,3,4a,5,6,7,8,8a-octahydropyrido[4,3-b][1,4]oxazin-4-yl]pyridazin-3-yl]-3,5-dimethyl-phenol;
(196) 3,5-dimethyl-2-[6-(2-oxa-6,9-diazaspiro[4.5]decan-9-yl)pyridazin-3-yl]phenol;
(197) 2-[6-[rac-(4aR,8aS)-2,3,4a,5,6,7,8,8a-octahydropyrido[4,3-b][1,4]oxazin-4-yl]pyridazin-3-yl]-3-methyl-5-(trifluoromethyl)phenol;
(198) 2-[6-[(2S,6S)-2,6-bis(hydroxymethyl)morpholin-4-yl]pyridazin-3-yl]-3,5-dimethyl-phenol;
(199) 2-[6-[(2S,6S)-2,6-bis(hydroxymethyl)morpholin-4-yl]pyridazin-3-yl]-3-methyl-5-(trifluoromethyl)phenol;
(200) 2-[6-[(2R,6R)-2,6-bis(hydroxymethyl)morpholin-4-yl]pyridazin-3-yl]-3-methyl-5-(trifluoromethyl)phenol;
(201) 2-[6-[(2R,6R)-2,6-bis(hydroxymethyl)morpholin-4-yl]pyridazin-3-yl]-3,5-dimethyl-phenol;
(203) 3,5-dichloro-2-(6-thiomorpholinopyridazin-3-yl)phenol;
(204) 5-cyclopropyl-2-[6-[(2S)-2-(hydroxymethyl)morpholin-4-yl]pyridazin-3-yl]-3-methyl-phenol;
(206) 2-[6-[(3aS,6aR)-1,3,3a,4,6,6a-hexahydrofuro[3,4-c]pyrrol-5-yl]pyridazin-3-yl]-3,5-dimethyl-phenol;
(207) 2-[6-[(3aR,6aR)-1,3,3a,4,6,6a-hexahydrofuro[3,4-c]pyrrol-5-yl]pyridazin-3-yl]-3,5-dimethyl-phenol;
(208) 2-[6-(8-ethyl-1,11-dioxa-4,8-diazaspiro[5.6]dodecan-4-yl)pyridazin-3-yl]-3,5-dimethylphenol;
(209) 2-[6-[rac-(4aR,8aS)-6-ethyl-3,4a,5,7,8,8a-hexahydro-2H-pyrido[4,3-b][1,4]oxazin-4-yl]pyridazin-3-yl]-3,5-dimethyl-phenol;
(210) 2-[6-[rac-(4aR,8aS)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol;
(211) N-[[(3S)-1-[6-(4-chloro-2-hydroxy-6-methyl-phenyl)pyridazin-3-yl]-3-piperidyl]methyl]acetamide;
(212) N-[[(3R)-1-[6-(4-chloro-2-hydroxy-6-methyl-phenyl)pyridazin-3-yl]-3-piperidyl]methyl]acetamide;
(213) 1-[rac-(4aR,8aS)-4-[6-(2-hydroxy-4,6-dimethyl-phenyl)pyridazin-3-yl]-3,4a,5,7,8,8a-hexahydro-2H-pyrido[4,3-b][1,4]oxazin-6-yl]ethanone;
(214) 1-[rac-(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(215) 2-[6-(2,3-dihydropyrido[3,4-b][1,4]oxazin-1-yl)pyridazin-3-yl]-3,5-dimethyl-phenol;
(216) N-[[(2R)-4-[6-[2-hydroxy-6-methyl-4-(trifluoromethyl)phenyl]pyridazin-3-yl]morpholin-2-yl]methyl]acetamide;
(217) 1-[6-(2-hydroxy-4,6-dimethyl-phenyl)pyridazin-3-yl]-3,4-dihydro-2H-quinolin-7-ol;
(219) 2-[6-[(2S)-2-(fluoromethyl)morpholin-4-yl]pyridazin-3-yl]-3,5-dimethyl-phenol;
(220) 2-[6-[(2R)-2-(fluoromethyl)morpholin-4-yl]pyridazin-3-yl]-3,5-dimethyl-phenol;
(221) 2-[6-[rac-(4aR,8aS)-2,3,4a,5,6,7,8,8a-octahydropyrido[4,3-b][1,4]oxazin-4-yl]pyridazin-3-yl]-5-chloro-3-methyl-phenol;
(222) 1-[rac-(4aR,8aS)-4-[6-(4-chloro-2-hydroxy-6-methyl-phenyl)pyridazin-3-yl]-3,4a,5,7,8,8a-hexahydro-2H-pyrido[4,3-b][1,4]oxazin-6-yl]ethanone;
(223) 2-[6-[rac-(4aR,8aS)-6-ethyl-3,4a,5,7,8,8a-hexahydro-2H-pyrido[4,3-b][1,4]oxazin-4-yl]pyridazin-3-yl]-5-chloro-3-methyl-phenol;
(224) 2-[6-[rac-(4aR,8aS)-6-methyl-3,4a,5,7,8,8a-hexahydro-2H-pyrido[4,3-b][1,4] oxazin-4-yl]pyridazin-3-yl]-5-chloro-3-methyl-phenol;
(225) 2-[6-[rac-(4aR,8aS)-6-methyl-3,4a,5,7,8,8a-hexahydro-2H-pyrido[4,3-b][1,4]oxazin-4-yl]pyridazin-3-yl]-3,5-dimethyl-phenol;
(226) 2-[6-[rac-(4aR,8aS)-6-methyl-3,4a,5,7,8,8a-hexahydro-2H-pyrido[4,3-b][1,4]oxazin-4-yl]pyridazin-3-yl]-3-methyl-5-(trifluoromethyl)phenol;
(227) 2-[6-(6-ethyl-2-oxa-6,9-diazaspiro[4.5]decan-9-yl)pyridazin-3-yl]-3,5-dimethylphenol;
(228) 1-[rac-(3aS,6aS)-1-[6-[2-hydroxy-6-methyl-4-(trifluoromethyl)phenyl]pyridazin-3-yl]-2,3,3a,4,6,6a-hexahydropyrrolo[3,4-b]pyrrol-5-yl]ethanone;
(229) 2-[6-[rac-(3aS,6aS)-3,3a,4,5,6,6a-hexahydro-2H-pyrrolo[3,4-b]pyrrol-1-yl]pyridazin-3-yl]-3-methyl-5-(trifluoromethyl)phenol;
(230) 1-[6-(2-hydroxy-4,6-dimethyl-phenyl)pyridazin-3-yl]-3,4-dihydro-2H-quinolin-6-ol;
(231) 1-[6-(2-hydroxy-4,6-dimethyl-phenyl)pyridazin-3-yl]-3,4-dihydro-2H-quinolin-8-ol;
(232) 1-[6-(2-hydroxy-4,6-dimethyl-phenyl)pyridazin-3-yl]-3,4-dihydro-2H-quinolin-5-ol;
(233) 2-[6-[(2R)-2-(2-hydroxyethyl)morpholin-4-yl]pyridazin-3-yl]-3,5-dimethyl-phenol;
(234) 2-[6-[(2S)-2-(2-hydroxyethyl)morpholin-4-yl]pyridazin-3-yl]-3,5-dimethyl-phenol;
(235) 1-[6-(2-hydroxy-4,6-dimethyl-phenyl)pyridazin-3-yl]-5-(hydroxymethyl) piperidin-2-one;
(236) 1-[6-(2-hydroxy-4,6-dimethyl-phenyl)pyridazin-3-yl]indolin-5-ol;
(237) 3,5-dichloro-2-[6-[(2R)-2-(fluoromethyl)morpholin-4-yl]pyridazin-3-yl]phenol;
(238) 3,5-dichloro-2-[6-[(2S)-2-(fluoromethyl)morpholin-4-yl]pyridazin-3-yl]phenol;
(239) 1-[6-(2,4-dichloro-6-hydroxy-phenyl)pyridazin-3-yl]hexahydropyrimidin-2-one;
(240) 1-[6-(2-hydroxy-4,6-dimethyl-phenyl)pyridazin-3-yl]indolin-4-ol;
(241) 2-[6-[4-(hydroxymethyl)indolin-1-yl]pyridazin-3-yl]-3,5-dimethyl-phenol;
(242) 1-[6-(2-hydroxy-4,6-dimethyl-phenyl)pyridazin-3-yl]-3,4-dihydro-2H-1,6-naphthyridin-5-ol;
(243) 2-[6-[rac-(3aS,6aS)-5-ethyl-2,3,3a,4,6,6a-hexahydropyrrolo[3,4-b]pyrrol-1-yl]pyridazin-3-yl]-3-methyl-5-(trifluoromethyl)phenol;
(245) 5-ethyl-2-[6-[(2S)-2-(hydroxymethyl)morpholin-4-yl]pyridazin-3-yl]-3-methylphenol;
(246) 3-ethyl-2-[6-[(2S)-2-(hydroxymethyl)morpholin-4-yl]pyridazin-3-yl]-5-methylphenol;
(247) 2-[4-(difluoromethyl)-6-[(2S)-2-(hydroxymethyl)morpholin-4-yl]pyridazin-3-yl]-5-(trifluoromethyl)phenol;
(248) 2-[4-(difluoromethyl)-6-[(2S)-2-(hydroxymethyl)morpholin-4-yl]pyridazin-3-yl]-3-methyl-5-(trifluoromethyl)phenol;
(249) 5-chloro-2-[4-(difluoromethyl)-6-[(2S)-2-(hydroxymethyl)morpholin-4-yl]pyridazin-3-yl]-3-methyl-phenol;
(250) (4S)-4-amino-1-[6-[2-hydroxy-6-methyl-4-(trifluoromethyl)phenyl]pyridazin-3-yl]pyrrolidin-2-one;
(251) 2-[6-[(4aR,8aS)-2,3,4a,5,6,7,8,8a-octahydropyrido[4,3-b][1,4]oxazin-4-yl]pyridazin-3-yl]-3-methyl-5-(trifluoromethyl)phenol;
(252) 2-[6-[(4aS,8aR)-2,3,4a,5,6,7,8,8a-octahydropyrido[4,3-b][1,4]oxazin-4-yl]pyridazin-3-yl]-3-methyl-5-(trifluoromethyl)phenol;
(253) 1-[6-(2-hydroxy-4,6-dimethyl-phenyl)pyridazin-3-yl]indolin-7-ol;
(254) 1-[6-(2-hydroxy-4,6-dimethyl-phenyl)pyridazin-3-yl]indolin-6-ol;
(255) 2-[6-[(4aR,8aS)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol;
(256) 2-[6-[(4aS,8aR)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol;
(257) 1-[rac-(4aS,7aS)-4-[6-(2-hydroxy-4,6-dimethylphenyl)pyridazin-3-yl]-2,3,4a,5,7,7a-hexahydropyrrolo[3,4-b][1,4]oxazin-6-yl]ethanone;
(258) 3-methyl-2-[6-[(2R)-2-methylmorpholin-4-yl]pyridazin-3-yl]-5-(trifluoromethyl)phenol;
(259) 3-methyl-2-[6-[(2S)-2-methylmorpholin-4-yl]pyridazin-3-yl]-5-(trifluoromethyl)phenol;
(260) 2-[6-[(4aR,8aS)-6-methyl-3,4a,5,7,8,8a-hexahydro-2H-pyrido[4,3-b][1,4]oxazin-4-yl]pyridazin-3-yl]-3-methyl-5-(trifluoromethyl)phenol;
(261) 2-[6-[(4aS,8aR)-6-methyl-3,4a,5,7,8,8a-hexahydro-2H-pyrido[4,3-b][1,4]oxazin-4-yl]pyridazin-3-yl]-3-methyl-5-(trifluoromethyl)phenol;
(262) 1-[(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(263) 1-[(4aS,8aR)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(264) rac-(3aS,7aR)-5-[6-[2-hydroxy-6-methyl-4-(trifluoromethyl)phenyl]pyridazin-3-yl]-2,3,3a,6,7,7a-hexahydro-1H-pyrrolo[3,4-c]pyridin-4-one, hydrochloride;
(265) 2-[6-[rac-(3aS,6aS)-5-methyl-2,3,3a,4,6,6a-hexahydropyrrolo[3,4-b]pyrrol-1-yl]pyridazin-3-yl]-3-methyl-5-(trifluoromethyl)phenol;
(266) 2-[6-[(2R)-2-(hydroxymethyl)morpholin-4-yl]pyridazin-3-yl]-3-methyl-5-(trifluoromethyl)phenol;
(267) N-[[(2R)-4-[6-(4-chloro-2-hydroxy-6-methyl-phenyl)pyridazin-3-yl]morpholin-2-yl]methyl]acetamide;
(268) 2-[6-[(4aR,8aS)-6-methyl-3,4a,5,7,8,8a-hexahydro-2H-pyrido[4,3-b][1,4]oxazin-4-yl]-4-(difluoromethyl)pyridazin-3-yl]-5-(trifluoromethyl)phenol;
(269) rac-(3aS,7aS)-5-[6-[2-hydroxy-6-methyl-4-(trifluoromethyl)phenyl]pyridazin-3-yl]-2,3,3a,6,7,7a-hexahydro-1H-pyrrolo[3,4-c]pyridin-4-one, hydrochloride;
(270) 2-[6-[(3aR,6aR)-5-methyl-2,3,3a,4,6,6a-hexahydropyrrolo[3,4-b]pyrrol-1-yl]pyridazin-3-yl]-3-methyl-5-(trifluoromethyl)phenol;
(271) 2-[6-[(3aS,6aS)-5-methyl-2,3,3a,4,6,6a-hexahydropyrrolo[3,4-b]pyrrol-1-yl]pyridazin-3-yl]-3-methyl-5-(trifluoromethyl)phenol;
(272) 2-[6-(3,4-dihydro-2H-quinolin-1-yl)pyridazin-3-yl]-3,5-dimethyl-phenol;
(273) 3-(hydroxymethyl)-1-[6-[2-hydroxy-6-methyl-4-(trifluoromethyl)phenyl]pyridazin-3-yl]piperidin-2-one;
(274) 2-[6-[(2S)-2-(hydroxymethyl)morpholin-4-yl]pyridazin-3-yl]-5-isopropyl-3-methyl-phenol;
(275) N-[[(2S)-4-[6-[2-hydroxy-6-methyl-4-(trifluoromethyl)phenyl]pyridazin-3-yl]morpholin-2-yl]methyl]acetamide;
(276) N-[[(2S)-4-[6-[2-hydroxy-6-methyl-4-(trifluoromethyl)phenyl]pyridazin-3-yl]morpholin-2-yl]methyl]-2-methylpropanamide;
(277) N-[2-[(2R)-4-[6-[2-hydroxy-6-methyl-4-(trifluoromethyl)phenyl]pyridazin-3-yl]morpholin-2-yl]ethyl]acetamide;
(278) N-[2-[(2S)-4-[6-[2-hydroxy-6-methyl-4-(trifluoromethyl)phenyl]pyridazin-3-yl]morpholin-2-yl]ethyl]acetamide;
(279) N-[[(2R)-4-[6-(4-chloro-2-hydroxy-6-methylphenyl)pyridazin-3-yl]morpholin-2-yl]methyl]-2-methylpropanamide;
(280) N-[[(2S)-4-[6-(4-chloro-2-hydroxy-6-methylphenyl)pyridazin-3-yl]morpholin-2-yl]methyl]acetamide.
(283) 2-[6-[(2R)-2-(fluoromethyl)morpholin-4-yl]pyridazin-3-yl]-3-methyl-5-(trifluoromethyl)phenol;
(284) 2-[6-[(2S)-2-(fluoromethyl)morpholin-4-yl]pyridazin-3-yl]-3-methyl-5-(trifluoromethyl)phenol;
(285) N-[[(2S)-4-[6-(4-chloro-2-hydroxy-6-methylphenyl)pyridazin-3-yl]morpholin-2-yl]methyl]-2-methylpropanamide;
(286) (4S)-4-hydroxy-1-[6-[2-hydroxy-6-methyl-4-(trifluoromethyl)phenyl]pyridazin-3-yl]piperidin-2-one;
(287) (3R)-3-(hydroxymethyl)-1-[6-[2-hydroxy-6-methyl-4-(trifluoromethyl)phenyl]pyridazin-3-yl]piperidin-2-one;
(288) (3S)-3-(hydroxymethyl)-1-[6-[2-hydroxy-6-methyl-4-(trifluoromethyl)phenyl]pyridazin-3-yl]piperidin-2-one;
(289) 3-methyl-2-[6-[rac-(4aR,7aR)-6-methyl-2,3,4a,5,7,7a-hexahydropyrrolo[3,4-b][1,4]oxazin-4-yl]pyridazin-3-yl]-5-(trifluoromethyl)phenol;
(290) (4aR,8aR)-1-[6-(2-hydroxy-4,6-dimethyl-phenyl)pyridazin-3-yl]-7-methyl-3,4,4a,5,6,8a-hexahydro-2H-1,7-naphthyridin-8-one;
(291) 2-[6-[(4aS,8aR)-6-methyl-3,4a,5,7,8,8a-hexahydro-2H-pyrido[4,3-b][1,4]oxazin-4-yl]-5-(difluoromethyl)pyridazin-3-yl]-3, 5-dimethyl-phenol;
(292) (4R)-4-(hydroxymethyl)-1-[6-[2-hydroxy-6-methyl-4-(trifluoromethyl)phenyl]pyridazin-3-yl]piperidin-2-one;
(293) (4S)-4-(hydroxymethyl)-1-[6-[2-hydroxy-6-methyl-4-(trifluoromethyl)phenyl]pyridazin-3-yl]piperidin-2-one;
(295) 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-5-methyl-phenol;
(296) 2-[6-[(2S,6R)-2-(hydroxymethyl)-6-methyl-morpholin-4-yl]pyridazin-3-yl]-3-methyl-5-(trifluoromethyl)phenol;
(297) 2-[6-[(2S,6S)-2-(hydroxymethyl)-6-methyl-morpholin-4-yl]pyridazin-3-yl]-3-methyl-5-(trifluoromethyl)phenol;
(298) 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-5-(trifluoromethyl)phenol;
(299) 2-[6-[(4aR,8aS)-6-methyl-3,4a,5,7,8,8a-hexahydro-2H-pyrido[4,3-b][1,4]oxazin-4-yl]pyridazin-3-yl]-3-methyl-phenol;
(300) 1-[6-(2-hydroxy-4,6-dimethyl-phenyl)pyridazin-3-yl]-2,3,4,4a,5,6,7,8a-octahydro-1,7-naphthyridin-8-one;
(301) 2-[6-[(4aR,8aR)-6-methyl-3,4a,5,7,8,8a-hexahydro-2H-pyrido[4,3-b][1,4]oxazin-4-yl]pyridazin-3-yl]-3-methyl-5-(trifluoromethyl)phenol;
(302) 2-[6-[(2R,6R)-2-(hydroxymethyl)-6-methyl-morpholin-4-yl]pyridazin-3-yl]-3-methyl-5-(trifluoromethyl)phenol;
(303) 3,5-dimethyl-2-[6-[(2S)-2-(sulfanylmethyl)morpholin-4-yl]pyridazin-3-yl]phenol;
(304) 1-[rac-(4aR,8aS)-4-[6-(4-chloro-2-hydroxy-6-methyl-phenyl)pyridazin-3-yl]-3,4a,5,7,8,8a-hexahydro-2H-pyrido[4,3-b][1,4]oxazin-6-yl]ethanone;
(305) 3,5-dimethyl-2-[6-[(2R)-2-(sulfanylmethyl)morpholin-4-yl]pyridazin-3-yl]phenol;
(306) 2-[6-[(4aR,8aS)-6-methyl-3,4a,5,7,8,8a-hexahydro-2H-pyrido[4,3-b][1,4]oxazin-4-yl]-4-(difluoromethyl)pyridazin-3-yl]-5-methyl-phenol;
(307) 5-chloro-3-methyl-2-[6-[rac-(4aR,8aS)-6-methyl-3,4a,5,7,8,8a-hexahydro-2H-pyrido[4,3-b][1,4]oxazin-4-yl]pyridazin-3-yl]phenol;
(308) 2-[6-[(4aR,8aS)-2,3,4a,5,6,7,8,8a-octahydropyrido[4,3-b][1,4]oxazin-4-yl]pyridazin-3-yl]-5-chloro-3-methyl-phenol;
(309) 2-[6-[(4aS,8aR)-2,3,4a,5,6,7,8,8a-octahydropyrido[4,3-b][1,4]oxazin-4-yl]pyridazin-3-yl]-5-chloro-3-methyl-phenol;
(310) 2-[6-[(4aR,8aS)-6-methyl-3,4a,5,7,8,8a-hexahydro-2H-pyrido[4,3-b][1,4]oxazin-4-yl]pyridazin-3-yl]-3,5-dimethyl-phenol;
(311) 2-[6-[(4aS,8aR)-6-methyl-3,4a,5,7,8,8a-hexahydro-2H-pyrido[4,3-b][1,4]oxazin-4-yl]pyridazin-3-yl]-3,5-dimethyl-phenol;
(312) 2-[6-[(4aR,8aS)-6-ethyl-3,4a,5,7,8,8a-hexahydro-2H-pyrido[4,3-b][1,4]oxazin-4-yl]pyridazin-3-yl]-5-chloro-3-methyl-phenol;
(313) 2-[6-[(4aS,8aR)-6-ethyl-3,4a,5,7,8,8a-hexahydro-2H-pyrido[4,3-b][1,4]oxazin-4-yl]pyridazin-3-yl]-5-chloro-3-methyl-phenol;
(314) 2-[6-[(4aR,8aS)-2,3,4a,5,6,7,8,8a-octahydropyrido[4,3-b][1,4]oxazin-4-yl]pyridazin-3-yl]-3,5-dimethyl-phenol;
(315) 2-[6-[(4aS,8aR)-2,3,4a,5,6,7,8,8a-octahydropyrido[4,3-b][1,4]oxazin-4-yl]pyridazin-3-yl]-3,5-dimethyl-phenol;
(316) (4R)-4-hydroxy-1-[6-[2-hydroxy-6-methyl-4-(trifluoromethyl)phenyl]pyridazin-3-yl]piperidin-2-one;
(317) 2-[6-(4-ethyl-1,11-dioxa-4,8-diazaspiro[5.6]dodecan-8-yl)pyridazin-3-yl]-3,5-dimethylphenol;
(318) 3-methyl-2-[6-[rac-(4aR,7aR)-3,4a,5,6,7,7a-hexahydro-2H-pyrrolo[3,4-b][1,4]oxazin-4-yl]pyridazin-3-yl]-5-(trifluoromethyl)phenol;hydrochloride;
(320) 3-methyl-2-[6-[rac-(4aR,7aS)-3,4a,5,6,7,7a-hexahydro-2H-pyrrolo[3,4-b][1,4]oxazin-4-yl]pyridazin-3-yl]-5-(trifluoromethyl)phenol;hydrochloride;
(321) 2-[6-[(4aS,8aR)-6-methyl-3,4a,5,7,8,8a-hexahydro-2H-pyrido[4,3-b][1,4]oxazin-4-yl]pyridazin-3-yl]-5-chloro-3-methyl-phenol;
(322) 2-[6-[(4aS,8aR)-2,3,4a,5,6,7,8,8a-octahydropyrido[4,3-b][1,4]oxazin-4-yl]-4-(difluoromethyl)pyridazin-3-yl]-5-methyl-phenol;
(323) 2-[6-[(4aS,8aR)-6-ethyl-3,4a,5,7,8,8a-hexahydro-2H-pyrido[4,3-b][1,4]oxazin-4-yl]-4-(difluoromethyl)pyridazin-3-yl]-5-methyl-phenol;
(324) 5-[6-[(2S)-2-(hydroxymethyl)morpholin-4-yl]-4-methyl-pyridazin-3-yl]indan-4-ol;
(325) (3R)-3-(hydroxymethyl)-1-[6-[2-hydroxy-6-methyl-4-(trifluoromethyl)phenyl]pyridazin-3-yl]pyrrolidin-2-one;
(326) (3S)-3-(hydroxymethyl)-1-[6-[2-hydroxy-6-methyl-4-(trifluoromethyl)phenyl]pyridazin-3-yl]pyrrolidin-2-one;
(327) 2-[(2R)-4-[6-[2-hydroxy-6-methyl-4-(trifluoromethyl)phenyl]pyridazin-3-yl]morpholin-2-yl]acetonitrile;
(328) 2-[(2S)-4-[6-[2-hydroxy-6-methyl-4-(trifluoromethyl)phenyl]pyridazin-3-yl]morpholin-2-yl]acetonitrile;
(329) (6R)-4-[6-(2-hydroxy-4,6-dimethyl-phenyl)pyridazin-3-yl]-6-(hydroxymethyl)piperazin-2-one;
(330) (6S)-4-[6-(2-hydroxy-4,6-dimethyl-phenyl)pyridazin-3-yl]-6-(hydroxymethyl)piperazin-2-one;
(331) 3-methyl-2-[6-[(2R)-2-[2-(methylamino)ethyl]morpholin-4-yl]pyridazin-3-yl]-5-(trifluoromethyl)phenol;
(332) 3-methyl-2-[6-[(2S)-2-[2-(methylamino)ethyl]morpholin-4-yl]pyridazin-3-yl]-5-(trifluoromethyl)phenol;
(333) 2-[6-[(4aR,7aS)-6-methyl-2,3,4a,5,7,7a-hexahydropyrrolo[3,4-b][1,4]oxazin-4-yl]pyridazin-3-yl]-3-methyl-5-(trifluoromethyl)phenol;
(334) 2-[6-[(4aS,7aR)-6-methyl-2,3,4a,5,7,7a-hexahydropyrrolo[3,4-b][1,4]oxazin-4-yl]pyridazin-3-yl]-3-methyl-5-(trifluoromethyl)phenol;
(335) 1-[rac-(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone;
(336) 1-[(4aR,8aR)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(337) 1-[rac-(4aR,7aR)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone;
(338) 5-chloro-2-[6-[(2R)-2-(fluoromethyl)morpholin-4-yl]pyridazin-3-yl]-3-methyl-phenol;
(339) 5-chloro-2-[6-[(2S)-2-(fluoromethyl)morpholin-4-yl]pyridazin-3-yl]-3-methyl-phenol;
(340) 2-[6-[(rac-3aS,7aR)-6-methyl-3,3a,4,5,7,7a-hexahydro-2H-pyrrolo[2,3-c]pyridin-1-yl]-4-methyl-pyridazin-3-yl]-5-(trifluoromethyl)phenol;
(341) 2-[6-[(rac-3aS,7aR)-6-methyl-3,3a,4,5,7,7a-hexahydro-2H-pyrrolo[2,3-c]pyridin-1-yl]-4-methyl-pyridazin-3-yl]-5-methyl-phenol;
(342) 3,5-dimethyl-2-[6-[rac-(3aS,7aR)-6-methyl-3,3a,4,5,7,7a-hexahydro-2H-pyrrolo[2,3-c]pyridin-1-yl]pyridazin-3-yl]phenol;
(343) 2-[6-[(4aS,8aS)-6-methyl-3,4a,5,7,8,8a-hexahydro-2H-pyrido[4,3-b][1,4]oxazin-4-yl]pyridazin-3-yl]-3-methyl-5-(trifluoromethyl)phenol;
(344) 2-[6-[(4aR,8aR)-6-ethyl-3,4a,5,7,8,8a-hexahydro-2H-pyrido[4,3-b][1,4]oxazin-4-yl]pyridazin-3-yl]-3-methyl-5-(trifluoromethyl)phenol;
(345) 5-[4-(difluoromethyl)-6-[(2S)-2-(hydroxymethyl)morpholin-4-yl]pyridazin-3-yl]indan-4-ol;
(346) 3-methyl-2-[6-[rac-(3aS,6aS)-2,3,3a,5,6,6a-hexahydro-1H-pyrrolo[3,2-b]pyrrol-4-yl]pyridazin-3-yl]-5-(trifluoromethyl)phenol;hydrochloride;
(348) 1-[(4aR,7aS)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone;
(349) 1-[(4aS,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(350) 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-5-chloro-phenol;
(351) 2-[6-[(4aS,8aR)-6-methyl-3,4a,5,7,8,8a-hexahydro-2H-pyrido[4,3-b][1,4]oxazin-4-yl]-4-(difluoromethyl)pyridazin-3-yl]-6-fluoro-phenol;
(352) 1-[(4aS,7aR)-4-[6-[2-hydroxy-6-methyl-4-(trifluoromethyl)phenyl]pyridazin-3-yl]-2,3,4a,5,7,7a-hexahydropyrrolo[3,4-b][1,4]oxazin-6-yl]ethanone;
(353) 2-[6-[(4aS,8aR)-6-methyl-3,4a,5,7,8,8a-hexahydro-2H-pyrido[4,3-b][1,4]oxazin-4-yl]pyridazin-3-yl]-6-fluoro-3-methyl-phenol;
(354) 2-[6-[(4aS,8aR)-6-methyl-3,4a,5,7,8,8a-hexahydro-2H-pyrido[4,3-b][1,4]oxazin-4-yl]pyridazin-3-yl]-3-methyl-phenol;
(355) 2-[6-[(4aS,8aR)-6-(2,2,2-trifluoroethyl)-3,4a,5,7,8,8a-hexahydro-2H-pyrido[4,3-b][1,4]oxazin-4-yl]pyridazin-3-yl]-3, 5-dimethyl-phenol;
(356) 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-6-fluoro-phenol;
(357) 1-[(4aS,8aR)-4-[6-(4-chloro-2-hydroxy-phenyl)-5-methyl-pyridazin-3-yl]-3,4a,5,7,8,8a-hexahydro-2H-pyrido[4,3-b][1,4]oxazin-6-yl]ethanone;
(358) 2-[6-[(4aS,8aR)-6-methyl-3,4a,5,7,8,8a-hexahydro-2H-pyrido[4,3-b][1,4]oxazin-4-yl]pyridazin-3-yl]-3-ethyl-phenol;
(359) 2-[6-[(2S,6R)-2-(hydroxymethyl)-6-methyl-morpholin-4-yl]pyridazin-3-yl]-3,5-dimethyl-phenol;
(360) 2-[6-(5,6-dihydro-2H-pyrazolo[4,3-b][1,4]oxazin-7-yl)pyridazin-3-yl]-3-methyl-5-(trifluoromethyl)phenol;
(361) 3-methyl-2-[6-[rac-(4aR,7aS)-6-ethyl-2,3,4a,5,7,7a-hexahydropyrrolo[3,4-b][1,4]oxazin-4-yl]pyridazin-3-yl]-5-(trifluoromethyl)phenol;
(362) 2-[6-(2,3-dihydropyrido[4,3-b][1,4]oxazin-4-yl)pyridazin-3-yl]-3,5-dimethyl-phenol;
(363) 1-[(4aS,8aR)-4-[6-[2-hydroxy-4-(trifluoromethyl)phenyl]-5-methyl-pyridazin-3-yl]-3,4a,5,7,8,8a-hexahydro-2H-pyrido[4,3-b][1,4]oxazin-6-yl]ethanone;
(364) 2-[6-[rac-(3aS,7aS)-6-methyl-3,3a,4,5,7,7a-hexahydro-2H-pyrrolo[2,3-c]pyridin-1-yl]pyridazin-3-yl]-3-methyl-5-(trifluoromethyl)phenol;
(365) 2-[6-[(2R,6R)-2-(hydroxymethyl)-6-methyl-morpholin-4-yl]pyridazin-3-yl]-3,5-dimethyl-phenol;
(366) 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-(trifluoromethyl)phenol;
(367) 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-(trifluoromethyl)phenol;
(368) 2-(hydroxymethyl)-4-[6-[2-hydroxy-6-methyl-4-(trifluoromethyl)phenyl]pyridazin-3-yl]morpholin-3-one;
(369) 1-[(3aR,7aR)-1-[6-[2-hydroxy-6-methyl-4-(trifluoromethyl)phenyl]pyridazin-3-yl]-3,3a,4,5,7,7a-hexahydro-2H-pyrrolo[2,3-c]pyridin-6-yl]ethanone;
(370) 1-[(3aS,7aS)-1-[6-[2-hydroxy-6-methyl-4-(trifluoromethyl)phenyl]pyridazin-3-yl]-3,3a,4,5,7,7a-hexahydro-2H-pyrrolo[2,3-c]pyridin-6-yl]ethanone;
(371) 2-[6-[rac-(3aS,7aR)-6-methyl-3,3a,4,5,7,7a-hexahydro-2H-pyrrolo[2,3-c]pyridin-1-yl]pyridazin-3-yl]-3-methyl-5-(trifluoromethyl)phenol;
(372) (4S)-1-[5-(difluoromethyl)-6-[2-hydroxy-6-methyl-4-(trifluoromethyl)phenyl]pyridazin-3-yl]-4-hydroxy-pyrrolidin-2-one;
(373) 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-methyl-phenol;
(374) 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-methyl-phenol;
(375) 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-methyl-phenol;
(376) 3-methyl-2-[6-[rac-(4aR,7aR)-6-ethyl-2,3,4a,5,7,7a-hexahydropyrrolo[3,4-b][1,4]oxazin-4-yl]pyridazin-3-yl]-5-(trifluoromethyl)phenol;
(377) 1-[rac-(3aS,6aS)-1-[6-[2-hydroxy-6-methyl-4-(trifluoromethyl)phenyl]pyridazin-3-yl]-2,3,3a,5, 6,6a-hexahydropyrrolo[3,2-b]pyrrol-4-yl]ethanone;
(378) 2-[6-[rac-(3aS,7aR)-6-ethyl-3,3a,4,5,7,7a-hexahydro-2H-pyrrolo[2,3-c]pyridin-1-yl]pyridazin-3-yl]-3-methyl-5-(trifluoromethyl)phenol;
(379) 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-chloro-phenol;
(380) 1-[(4aS,8aR)-4-[6-[2-ethyl-6-hydroxy-4-(trifluoromethyl)phenyl]pyridazin-3-yl]-3,4a,5,7,8,8a-hexahydro-2H-pyrido[4,3-b][1,4]oxazin-6-yl]ethanone;
(381) 1-[(4aS,8aR)-4-[6-(4-chloro-2-ethyl-6-hydroxy-phenyl)pyridazin-3-yl]-3,4a,5,7,8,8a-hexahydro-2H-pyrido[4,3-b][1,4]oxazin-6-yl]ethanone;
(382) 2-[6-[(4aS,8aR)-2,3,4a,5,6,7,8,8a-octahydropyrido[4,3-b][1,4]oxazin-4-yl]-4-(difluoromethyl)pyridazin-3-yl]-5-(trifluoromethyl)phenol;
(383) 2-[6-[(3aR,7aS)-6-methyl-3,3a,4,5,7,7a-hexahydro-2H-pyrrolo[2,3-c]pyridin-1-yl]pyridazin-3-yl]-3,5-dimethyl-phenol;
(384) 2-[6-[(3aS,7aR)-6-methyl-3,3a,4,5,7,7a-hexahydro-2H-pyrrolo[2,3-c]pyridin-1-yl]pyridazin-3-yl]-3,5-dimethyl-phenol;
(385) 2-[6-[(3aS,7aR)-6-methyl-3,3a,4,5,7,7a-hexahydro-2H-pyrrolo[2,3-c]pyridin-1-yl]pyridazin-3-yl]-3-methyl-phenol;
(386) 1-[(4aS,8aR)-4-[5-(difluoromethyl)-6-[2-hydroxy-4-(trifluoromethyl)phenyl]pyridazin-3-yl]-3,4a,5,7,8,8a-hexahydro-2H-pyrido[4,3-b][1,4]oxazin-6-yl]ethanone;
(387) 1-[(4aS,8aR)-4-[5-(difluoromethyl)-6-(2-hydroxy-4-methyl-phenyl)pyridazin-3-yl]-3,4a,5,7,8,8a-hexahydro-2H-pyrido[4,3-b][1,4]oxazin-6-yl]ethanone;
(388) 2-[6-[(3aS,7aR)-6-methyl-3,3a,4,5,7,7a-hexahydro-2H-pyrrolo[2,3-c]pyridin-1-yl]pyridazin-3-yl]-3-ethyl-phenol;
(389) 2-[6-[(3aS,7aR)-6-methyl-3,3a,4,5,7,7a-hexahydro-2H-pyrrolo[2,3-c]pyridin-1-yl]pyridazin-3-yl]-3-(trifluoromethyl)phenol;
(390) 2-[6-[(2S,6S)-2-(hydroxymethyl)-6-methyl-morpholin-4-yl]pyridazin-3-yl]-3,5-dimethyl-phenol;
(391) 2-[6-[(3aR,7aS)-6-methyl-3,3a,4,5,7,7a-hexahydro-2H-pyrrolo[2,3-c]pyridin-1-yl]pyridazin-3-yl]-3-methyl-5-(trifluoromethyl)phenol;
(392) 2-[6-[(3aS,7aR)-6-methyl-3,3a,4,5,7,7a-hexahydro-2H-pyrrolo[2,3-c]pyridin-1-yl]pyridazin-3-yl]-3-methyl-5-(trifluoromethyl)phenol;
(393) 2-[6-[(3aR,7aS)-2,3,3a,4,5,6,7,7a-octahydropyrrolo[2,3-c]pyridin-1-yl]pyridazin-3-yl]-3-methyl-5-(trifluoromethyl)phenol;
(394) 1-[(3aR,7aR)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethanone;
(395) 1-[(3aR,7aR)-1-[6-[2-hydroxy-6-methyl-4-(trifluoromethyl)phenyl]pyridazin-3-yl]-3,3a,4,5,7,7a-hexahydro-2H-pyrrolo[2,3-c]pyridin-6-yl]ethanone;
(397) 1-[(4aS,8aR)-4-[6-(4-chloro-2-hydroxy-6-methyl-phenyl)pyridazin-3-yl]-3,4a,5,7,8,8a-hexahydro-2H-pyrido[4,3-b][1,4]oxazin-6-yl]ethanone;
(398) 1-[(4aR,8aS)-4-[6-[2-hydroxy-6-methyl-4-(trifluoromethyl)phenyl]pyridazin-3-yl]-3,4a,5,7,8,8a-hexahydro-2H-pyrido[4,3-b][1,4]oxazin-6-yl]ethanone;
(399) 3,5-dichloro-2-[6-(1,1-dioxo-1,4-thiazinan-4-yl)pyridazin-3-yl]phenol;
(400) 3,5-dichloro-2-[6-(1-oxo-1,4-thiazinan-4-yl)pyridazin-3-yl]phenol;
(402) 2-[6-[(4aS,8aR)-6-methyl-3,4a,5,7,8,8a-hexahydro-2H-pyrido[4,3-b][1,4]oxazin-4-yl]pyridazin-3-yl]-3-(trifluoromethyl)phenol;
(403) 3,5-dimethyl-2-[6-[rac-(4aS,8aR)-3,4a,5,7,8,8a-hexahydro-2H-pyrano[4,3-b][1,4]oxazin-4-yl]pyridazin-3-yl]phenol;
(404) 2-[6-[(4aS,8aR)-6-ethyl-3,4a,5,7,8,8a-hexahydro-2H-pyrido[4,3-b][1,4]oxazin-4-yl]pyridazin-3-yl]-3,5-dimethyl-phenol;
(405) 1-[(4aS,8aR)-4-[4-(difluoromethyl)-6-(2-hydroxy-4,6-dimethyl-phenyl)pyridazin-3-yl]-3,4a,5,7,8,8a-hexahydro-2H-pyrido[4,3-b][1,4]oxazin-6-yl]ethanone;
(406) 2-[6-[(3aS,7aR)-6-ethyl-3,3a,4,5,7,7a-hexahydro-2H-pyrrolo[2,3-c]pyridin-1-yl]pyridazin-3-yl]-3-methyl-5-(trifluoromethyl)phenol;
(407) 2-[6-[(3aS,7aR)-2,3,3a,4,5,6,7,7a-octahydropyrrolo[2,3-c]pyridin-1-yl]pyridazin-3-yl]-3-methyl-5-(trifluoromethyl)phenol;
(408) 2-[6-[(4aS,8aR)-6-ethyl-3,4a,5,7,8,8a-hexahydro-2H-pyrido[4,3-b][1,4]oxazin-4-yl]pyridazin-3-yl]-3-ethyl-phenol;
(409) 1-[(4aS,8aR)-4-[6-(2-hydroxy-6-methyl-phenyl)pyridazin-3-yl]-3,4a,5,7,8,8a-hexahydro-2H-pyrido[4,3-b][1,4]oxazin-6-yl]ethanone;
(410) 3,5-dimethyl-2-[6-(3-methyl-3,6-diazabicyclo[3.2.2]nonan-6-yl)pyridazin-3-yl]phenol;
(411) 2-[6-[(4aS,8aR)-2,3,4a,5,6,7,8,8a-octahydropyrido[4,3-b][1,4]oxazin-4-yl]pyridazin-3-yl]-3-methyl-phenol;
(412) 2-[6-[(4aS,8aR)-6-ethyl-3,4a,5,7,8,8a-hexahydro-2H-pyrido[4,3-b][1,4]oxazin-4-yl]pyridazin-3-yl]-3-methyl-phenol;
(413) 1-[(3aR,7aR)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethanone;
(414) 1-[(3aS,7aS)-1-[6-(2-hydroxy-4,6-dimethyl-phenyl)pyridazin-3-yl]-3,3a,4,5,7,7a-hexahydro-2H-pyrrolo[2,3-c]pyridin-6-yl]ethanone;
(415) 2-[6-[(3aS,7aR)-6-methyl-3,3a,4,5,7,7a-hexahydro-2H-pyrrolo[2,3-c]pyridin-1-yl]pyridazin-3-yl]-3-methyl-phenol;
(416) 2-[6-[(3aR,7aS)-6-methyl-3,3a,4,5,7,7a-hexahydro-2H-pyrrolo[2,3-c]pyridin-1-yl]pyridazin-3-yl]-3-methyl-phenol;
(417) 3-methyl-2-[6-(3-methyl-3,6-diazabicyclo[3.2.2]nonan-6-yl)pyridazin-3-yl]-5-(trifluoromethyl)phenol;
(418) 3-ethyl-2-[6-[(2S)-2-(hydroxymethyl)morpholin-4-yl]pyridazin-3-yl]-5-(trifluoromethyl)phenol;
(419) (2S)-2-(hydroxymethyl)-4-[6-[2-hydroxy-6-methyl-4-(trifluoromethyl)phenyl]pyridazin-3-yl]morpholin-3-one;
(420) (2R)-2-(hydroxymethyl)-4-[6-[2-hydroxy-6-methyl-4-(trifluoromethyl)phenyl]pyridazin-3-yl]morpholin-3-one;
(421) 3,5-dimethyl-2-[6-[rac-(4aS,8aS)-3,4a,5,7,8,8a-hexahydro-2H-pyrano[4,3-b][1,4]oxazin-4-yl]pyridazin-3-yl]phenol;
(422) 2-[6-[(3aS,7aR)-6-methyl-3,3a,4,5,7,7a-hexahydro-2H-pyrrolo[2,3-c]pyridin-1-yl]pyridazin-3-yl]-3-ethyl-phenol;
(423) 2-[6-[(3aR,7aS)-6-methyl-3,3a,4,5,7,7a-hexahydro-2H-pyrrolo[2,3-c]pyridin-1-yl]pyridazin-3-yl]-3-ethyl-phenol;
(424) 1-[(4aS,8aR)-4-[6-(2-ethyl-6-hydroxy-phenyl)pyridazin-3-yl]-3,4a,5,7,8,8a-hexahydro-2H-pyrido[4,3-b][1,4]oxazin-6-yl]ethanone;
(425) 2-[6-[(4aS,8aR)-6-methyl-3,4a,5,7,8,8a-hexahydro-2H-pyrido[4,3-b][1,4]oxazin-4-yl]-5-(difluoromethyl)pyridazin-3-yl]-3-ethyl-phenol;
(426) 2-[6-[(3aR,7aS)-6-methyl-3,3a,4,5,7,7a-hexahydro-2H-pyrrolo[2,3-c]pyridin-1-yl]pyridazin-3-yl]-3-(trifluoromethyl)phenol;
(427) 2-[6-[rac-(3aS,7aR)-6-methyl-3,3a,4,5,7,7a-hexahydro-2H-pyrrolo[2,3-c]pyridin-1-yl]pyridazin-3-yl]-3-(trifluoromethyl)phenol;
(428) 3,5-dimethyl-2-[6-(9-methyl-1,9-diazaspiro[4.5]decan-1-yl)pyridazin-3-yl]phenol;
(429) 2-[6-[(3aR,7aS)-6-ethyl-3,3a,4,5,7,7a-hexahydro-2H-pyrrolo[2,3-c]pyridin-1-yl]pyridazin-3-yl]-3-methyl-5-(trifluoromethyl)phenol;
(430) 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-(trifluoromethyl)phenol;
(431) 1-[(4aS,8aR)-4-[6-(2-hydroxy-4-methyl-phenyl)-5-methyl-pyridazin-3-yl]-3,4a,5,7,8,8a-hexahydro-2H-pyrido[4,3-b][1,4]oxazin-6-yl]ethanone;
(432) 2-[6-[(4aS,8aR)-6-isopropyl-3,4a,5,7,8,8a-hexahydro-2H-pyrido[4,3-b][1,4]oxazin-4-yl]pyridazin-3-yl]-3-ethyl-phenol;
(433) 2-[6-[(4aS,8aR)-6-isopropyl-3,4a,5,7,8,8a-hexahydro-2H-pyrido[4,3-b][1,4]oxazin-4-yl]pyridazin-3-yl]-3, 5-dimethyl-phenol;
(434) 2-[6-[(4aS,8aR)-6-methyl-3,4a,5,7,8,8a-hexahydro-2H-pyrido[4,3-b][1,4]oxazin-4-yl]-5-methyl-pyridazin-3-yl]-3,5-dimethyl-phenol;
(435) 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-methyl-phenol;
(436) 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-(trifluoromethyl)phenol;
(437) 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-chloro-phenol;
(438) 3-methyl-2-[6-[rac-(4aS,8aR)-3,4a,5,7,8,8a-hexahydro-2H-pyrano[4,3-b][1,4]oxazin-4-yl]pyridazin-3-yl]-5-(trifluoromethyl)phenol;
(439) 2-[6-[(4aS,8aR)-6-methyl-3,4a,5,7,8,8a-hexahydro-2H-pyrido[4,3-b][1,4]oxazin-4-yl]-5-methyl-pyridazin-3-yl]-3-ethyl-phenol;
(440) 3,5-dimethyl-2-[6-(9-methyl-6-oxa-2,9-diazaspiro[4.5]decan-2-yl)pyridazin-3-yl]phenol;
(441) 3,5-dimethyl-2-[6-(1-methyl-4-oxa-1,8-diazaspiro[5.5]undecan-8-yl)pyridazin-3-yl]phenol;
(442) 1-[(4aS,8aR)-4-[6-(4-chloro-2-hydroxy-phenyl)-5-(difluoromethyl)pyridazin-3-yl]-3,4a,5,7,8,8a-hexahydro-2H-pyrido[4,3-b][1,4]oxazin-6-yl]ethanone;
(443) 2-[6-[(4aS,8aR)-6-methyl-3,4a,5,7,8,8a-hexahydro-2H-pyrido[4,3-b][1,4]oxazin-4-yl]-4-ethyl-pyridazin-3-yl]-5-(trifluoromethyl)phenol;
(444) 3,5-dimethyl-2-[6-(6-methyl-9-oxa-2,6-diazaspiro[4.5]decan-2-yl)pyridazin-3-yl]phenol;
(445) 3,5-dimethyl-2-[6-[(5S)-9-methyl-6-oxa-2,9-diazaspiro[4.5]decan-2-yl]pyridazin-3-yl]phenol;
(446) 3,5-dimethyl-2-[6-[(5R)-9-methyl-6-oxa-2,9-diazaspiro[4.5]decan-2-yl]pyridazin-3-yl]phenol;
(448) 3-methyl-2-[6-[rac-(4aS,8aS)-3,4a,5,7,8,8a-hexahydro-2H-pyrano[4,3-b][1,4]oxazin-4-yl]pyridazin-3-yl]-5-(trifluoromethyl)phenol;
(449) 3,5-dimethyl-2-[6-(8-methyl-4-oxa-1,8-diazaspiro[5.5]undecan-1-yl)pyridazin-3-yl]phenol;
(450) 3,5-dimethyl-2-[6-[(5R)-6-methyl-9-oxa-2,6-diazaspiro[4.5]decan-2-yl]pyridazin-3-yl]phenol;
(451) 3,5-dimethyl-2-[6-[(5S)-6-methyl-9-oxa-2,6-diazaspiro[4.5]decan-2-yl]pyridazin-3-yl]phenol;
(452) 2-[6-[rac-(3aS,7aR)-6-methyl-3,3a,4,5,7,7a-hexahydro-2H-pyrrolo[2,3-c]pyridin-1-yl]pyridazin-3-yl]-3-ethyl-5-(trifluoromethyl)phenol;
(455) 3,5-dimethyl-2-[6-(2-methyl-8-oxa-2,5-diazaspiro[3.5]nonan-5-yl)pyridazin-3-yl]phenol;
(456) 2-[6-[(4aS,8aR)-6-ethyl-3,4a,5,7,8,8a-hexahydro-2H-pyrido[4,3-b][1,4]oxazin-4-yl]-4-(difluoromethyl)pyridazin-3-yl]-5-chloro-phenol;
(457) 1-[(3aR,7aR)-1-[6-(2-hydroxy-4-methyl-phenyl)-5-methyl-pyridazin-3-yl]-3,3a,4,5,7,7a-hexahydro-2H-pyrrolo[2,3-c]pyridin-6-yl]ethanone;
(458) 2-[6-[rac-(3aS,7aR)-6-ethyl-3,3a,4,5,7,7a-hexahydro-2H-pyrrolo[2,3-c]pyridin-1-yl]pyridazin-3-yl]-3-(trifluoromethyl)phenol;
(459) 2-[6-[rac-(3aS,7aR)-6-methyl-3,3a,4,5,7,7a-hexahydro-2H-pyrrolo[2,3-c]pyridin-1-yl]pyridazin-3-yl]-3-ethyl-5-methyl-phenol;
(460) 2-[6-[rac-(3aS,7aR)-6-ethyl-3,3a,4,5,7,7a-hexahydro-2H-pyrrolo[2,3-c]pyridin-1-yl]-4-methyl-pyridazin-3-yl]-5-methyl-phenol;
(461) 2-[6-[(4aS,8aR)-6-methyl-3,4a,5,7,8,8a-hexahydro-2H-pyrido[4,3-b][1,4]oxazin-4-yl]pyridazin-3-yl]-5-chloro-3-ethyl-phenol;
(462) 1-[(4aR,8aR)-5-[6-[2-hydroxy-6-methyl-4-(trifluoromethyl)phenyl]pyridazin-3-yl]-2,3,4,4a,6,7,8,8a-octahydro-1,5-naphthyridin-1-yl]ethanone;
(463) 1-[(4aS,8aS)-5-[6-[2-hydroxy-6-methyl-4-(trifluoromethyl)phenyl]pyridazin-3-yl]-2,3,4,4a,6,7,8,8a-octahydro-1,5-naphthyridin-1-yl]ethanone;
(466) (4aS,6S,7aR)-4-[6-(2-hydroxy-4,6-dimethyl-phenyl)pyridazin-3-yl]-3,4a,5,6,7,7a-hexahydro-2H-cyclopenta[b][1,4]oxazin-6-ol;
(467) (4aR,6R,7aS)-4-[6-(2-hydroxy-4,6-dimethyl-phenyl)pyridazin-3-yl]-3,4a,5,6,7,7a-hexahydro-2H-cyclopenta[b][1,4]oxazin-6-ol;
(468) 2-[6-[(3aR,7aS)-6-methyl-3,3a,4,5,7,7a-hexahydro-2H-pyrrolo[2,3-c]pyridin-1-yl]-4-methyl-pyridazin-3-yl]-5-chloro-phenol;
(469) 2-[6-[(3aS,7aR)-6-methyl-3,3a,4,5,7,7a-hexahydro-2H-pyrrolo[2,3-c]pyridin-1-yl]-4-methyl-pyridazin-3-yl]-5-chloro-phenol;
(470) 2-[6-[(4aS,8aR)-6-methyl-3,4a,5,7,8,8a-hexahydro-2H-pyrido[4,3-b][1,4]oxazin-4-yl]-4-ethyl-pyridazin-3-yl]-5-methyl-phenol;
(471) 5-[6-[(4aS,8aR)-6-methyl-3,4a,5,7,8,8a-hexahydro-2H-pyrido[4,3-b][1,4]oxazin-4-yl]-4-methyl-pyridazin-3-yl]indan-4-ol;
(472) 2-[6-[(4aS,8aR)-6-isopropyl-3,4a,5,7,8,8a-hexahydro-2H-pyrido[4,3-b][1,4]oxazin-4-yl]-4-methyl-pyridazin-3-yl]-5-methyl-phenol;
(473) 2-[6-[(3aS,7aR)-6-ethyl-3,3a,4,5,7,7a-hexahydro-2H-pyrrolo[2,3-c]pyridin-1-yl]pyridazin-3-yl]-3-(trifluoromethyl)phenol;
(474) 2-[6-[(3aR,7aS)-6-ethyl-3,3a,4,5,7,7a-hexahydro-2H-pyrrolo[2,3-c]pyridin-1-yl]-4-methyl-pyridazin-3-yl]-5-methyl-phenol;
(475) 2-[6-[(3aS,7aR)-6-ethyl-3,3a,4,5,7,7a-hexahydro-2H-pyrrolo[2,3-c]pyridin-1-yl]-4-methyl-pyridazin-3-yl]-5-methyl-phenol;
(476) 2-[6-[(3aR,7aS)-6-methyl-3,3a,4,5,7,7a-hexahydro-2H-pyrrolo[2,3-c]pyridin-1-yl]pyridazin-3-yl]-3-ethyl-5-methyl-phenol;
(477) 2-[6-[(3aS,7aR)-6-methyl-3,3a,4,5,7,7a-hexahydro-2H-pyrrolo[2,3-c]pyridin-1-yl]pyridazin-3-yl]-3-ethyl-5-methyl-phenol;
(479) 2-[6-[(4aS,8aR)-6-ethyl-3,4a,5,7,8,8a-hexahydro-2H-pyrido[4,3-b][1,4]oxazin-4-yl]-4-methyl-pyridazin-3-yl]-5-fluoro-phenol;
(480) 2-[6-[(4aS,8aR)-6-methyl-3,4a,5,7,8,8a-hexahydro-2H-pyrido[4,3-b][1,4]oxazin-4-yl]-4-methyl-pyridazin-3-yl]-5-fluoro-phenol;
(481) 2-[6-[(4aS,8aR)-6-methyl-3,4a,5,7,8,8a-hexahydro-2H-pyrido[4,3-b][1,4]oxazin-4-yl]pyridazin-3-yl]-3-ethyl-5-(trifluoromethyl)phenol;
(482) 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-(trifluoromethyl)phenol;
(483) 2-[6-[(3aS,7aR)-6-methyl-3,3a,4,5,7,7a-hexahydro-2H-pyrrolo[2,3-c]pyridin-1-yl]-4-(difluoromethyl)pyridazin-3-yl]-5-(trifluoromethyl)phenol;
(484) 3-methyl-2-[6-[rac-(3aS,6aS)-1-methyl-2,3,3a,5,6,6a-hexahydropyrrolo[3,2-b]pyrrol-4-yl]pyridazin-3-yl]-5-(trifluoromethyl)phenol;
(485) 2-[6-[(3aR,7aS)-6-ethyl-3,3a,4,5,7,7a-hexahydro-2H-pyrrolo[2,3-c]pyridin-1-yl]-4-(difluoromethyl)pyridazin-3-yl]-5-(trifluoromethyl)phenol;
(486) 2-[6-[(3aS,7aR)-6-ethyl-3,3a,4,5,7,7a-hexahydro-2H-pyrrolo[2,3-c]pyridin-1-yl]-4-(difluoromethyl)pyridazin-3-yl]-5-(trifluoromethyl)phenol;
(487) 2-[6-[(3aR,7aS)-6-methyl-3,3a,4,5,7,7a-hexahydro-2H-pyrrolo[2,3-c]pyridin-1-yl]-5-(difluoromethyl)pyridazin-3-yl]-3,5-dimethyl-phenol;
(488) 2-[6-[(3aS,7aR)-6-methyl-3,3a,4,5,7,7a-hexahydro-2H-pyrrolo[2,3-c]pyridin-1-yl]-5-(difluoromethyl)pyridazin-3-yl]-3,5-dimethyl-phenol;
(489) 2-[6-[(3aR,7aS)-6-ethyl-3,3a,4,5,7,7a-hexahydro-2H-pyrrolo[2,3-c]pyridin-1-yl]-4-(difluoromethyl)pyridazin-3-yl]-5-methyl-phenol;
(490) 2-[6-[(3aS,7aR)-6-ethyl-3,3a,4,5,7,7a-hexahydro-2H-pyrrolo[2,3-c]pyridin-1-yl]-4-(difluoromethyl)pyridazin-3-yl]-5-methyl-phenol;
(491) 2-[6-[(3aR,7aS)-6-methyl-3,3a,4,5,7,7a-hexahydro-2H-pyrrolo[2,3-c]pyridin-1-yl]pyridazin-3-yl]-3-ethyl-5-(trifluoromethyl)phenol;
(492) 2-[6-[(3aS,7aR)-6-methyl-3,3a,4,5,7,7a-hexahydro-2H-pyrrolo[2,3-c]pyridin-1-yl]pyridazin-3-yl]-3-ethyl-5-(trifluoromethyl)phenol;
(493) 2-[6-[(3aR,7aS)-6-methyl-3,3a,4,5,7,7a-hexahydro-2H-pyrrolo[2,3-c]pyridin-1-yl]pyridazin-3-yl]-5-chloro-3-ethyl-phenol (494) 2-[6-[(3aS,7aR)-6-methyl-3,3a,4,5,7,7a-hexahydro-2H-pyrrolo[2,3-c]pyridin-1-yl]pyridazin-3-yl]-5-chloro-3-ethyl-phenol;
(496) 2-[6-[(3aR,7aS)-6-ethyl-3,3a,4,5,7,7a-hexahydro-2H-pyrrolo[2,3-c]pyridin-1-yl]pyridazin-3-yl]-5-chloro-3-methyl-phenol;
(497) 2-[6-[(3aS,7aR)-6-ethyl-3,3a,4,5,7,7a-hexahydro-2H-pyrrolo[2,3-c]pyridin-1-yl]pyridazin-3-yl]-5-chloro-3-methyl-phenol;
(498) 2-[6-[(3aS,7aR)-6-ethyl-3,3a,4,5,7,7a-hexahydro-2H-pyrrolo[2,3-c]pyridin-1-yl]pyridazin-3-yl]-3-methyl-phenol;
(499) 2-[6-[(3aR,7aS)-6-ethyl-3,3a,4,5,7,7a-hexahydro-2H-pyrrolo[2,3-c]pyridin-1-yl]pyridazin-3-yl]-3-methyl-phenol;
(500) 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-chloro-phenol;
(501) 2-[6-[(3aS,7aR)-6-methyl-3,3a,4,5,7,7a-hexahydro-2H-pyrrolo[2,3-c]pyridin-1-yl]-4-(difluoromethyl)pyridazin-3-yl]-5-chloro-phenol;
(502) 2-[6-[(3aS,7aR)-6-methyl-3,3a,4,5,7,7a-hexahydro-2H-pyrrolo[2,3-c]pyridin-1-yl]pyridazin-3-yl]-5-chloro-3-methyl-phenol;
(503) 2-[6-[(3aR,7aS)-6-methyl-3,3a,4,5,7,7a-hexahydro-2H-pyrrolo[2,3-c]pyridin-1-yl]pyridazin-3-yl]-5-chloro-3-methyl-phenol;
(504) 2-[6-[(3aS,7aR)-6-ethyl-3,3a,4,5,7,7a-hexahydro-2H-pyrrolo[2,3-c]pyridin-1-yl]pyridazin-3-yl]-3,5-dimethyl-phenol;
(505) 2-[6-[(3aR,7aS)-6-ethyl-3,3a,4,5,7,7a-hexahydro-2H-pyrrolo[2,3-c]pyridin-1-yl]pyridazin-3-yl]-3,5-dimethyl-phenol;
(506) 2-[6-[(3aR,7aS)-6-methyl-3,3a,4,5,7,7a-hexahydro-2H-pyrrolo[2,3-c]pyridin-1-yl]-4-(difluoromethyl)pyridazin-3-yl]-5-methyl-phenol;
(507) 2-[6-[(3aS,7aR)-6-methyl-3,3a,4,5,7,7a-hexahydro-2H-pyrrolo[2,3-c]pyridin-1-yl]-4-(difluoromethyl)pyridazin-3-yl]-5-methyl-phenol;
(508) 5-ethynyl-2-[6-[(2S)-2-(hydroxymethyl)morpholin-4-yl]pyridazin-3-yl]-3-methyl-phenol;
(509) 2-[6-[(2S)-2-(hydroxymethyl)morpholin-4-yl]pyridazin-3-yl]-5-methyl-3-(trifluoromethyl)phenol;
(510) 1-[(3aS,7aS)-1-[5-(difluoromethyl)-6-[2-hydroxy-4-(trifluoromethyl)phenyl]pyridazin-3-yl]-3,3a,4,5,7,7a-hexahydro-2H-pyrrolo[2,3-c]pyridin-6-yl]ethanone;
(511) 1-[(3aR,7aR)-1-[5-(difluoromethyl)-6-[2-hydroxy-4-(trifluoromethyl)phenyl]pyridazin-3-yl]-3,3a,4,5,7,7a-hexahydro-2H-pyrrolo[2,3-c]pyridin-6-yl]ethanone;
(512) 1-[(3aS,7aS)-1-[4-(difluoromethyl)-6-(2-hydroxy-4,6-dimethyl-phenyl)pyridazin-3-yl]-3,3a,4,5,7,7a-hexahydro-2H-pyrrolo[2,3-c]pyridin-6-yl]ethanone;
(513) 1-[(3aR,7aR)-1-[4-(difluoromethyl)-6-(2-hydroxy-4,6-dimethyl-phenyl)pyridazin-3-yl]-3,3a,4,5,7,7a-hexahydro-2H-pyrrolo[2,3-c]pyridin-6-yl]ethanone;
(514) 1-[(3aR,7aR)-1-[6-[2-hydroxy-4-(trifluoromethyl)phenyl]-5-methyl-pyridazin-3-yl]-3,3a,4,5,7,7a-hexahydro-2H-pyrrolo[2,3-c]pyridin-6-yl]ethanone;
(515) 2-[6-[(4aS,8aS)-5-methyl-2,3,4,4a,6,7,8,8a-octahydro-1,5-naphthyridin-1-yl]pyridazin-3-yl]-3-methyl-5-(trifluoromethyl)phenol;
(516) 2-[6-[(4aR,8aR)-5-methyl-2,3,4,4a,6,7,8,8a-octahydro-1,5-naphthyridin-1-yl]pyridazin-3-yl]-3-methyl-5-(trifluoromethyl)phenol;
(517) 2-[6-[(4aR,8aS)-7-methyl-2,3,4,4a,5,6,8,8a-octahydro-1,7-naphthyridin-1-yl]pyridazin-3-yl]-3,5-dimethyl-phenol;
(518) 2-[6-[(4aS,8aS)-7-methyl-2,3,4,4a,5,6,8,8a-octahydro-1,7-naphthyridin-1-yl]pyridazin-3-yl]-3,5-dimethyl-phenol;
(519) 2-[6-[(4aR,8aR)-7-methyl-2,3,4,4a,5,6,8,8a-octahydro-1,7-naphthyridin-1-yl]pyridazin-3-yl]-3,5-dimethyl-phenol;
(520) 2-[6-[(4aS,8aR)-7-methyl-2,3,4,4a,5,6,8,8a-octahydro-1,7-naphthyridin-1-yl]pyridazin-3-yl]-3,5-dimethyl-phenol;
(521) 2-[6-[(4aS,8aR)-6-(2-fluoroethyl)-3,4a,5,7,8,8a-hexahydro-2H-pyrido[4,3-b][1,4]oxazin-4-yl]pyridazin-3-yl]-3, 5-dimethyl-phenol;
(522) 2-[6-[(4aS,8aR)-6-(2-fluoroethyl)-3,4a,5,7,8,8a-hexahydro-2H-pyrido[4,3-b][1,4]oxazin-4-yl]pyridazin-3-yl]-5-chloro-3-methyl-phenol;
(523) 2-[6-[(4aR,8aR)-7-methyl-2,3,4,4a,5,6,8,8a-octahydro-1,7-naphthyridin-1-yl]pyridazin-3-yl]-3-methyl-5-(trifluoromethyl)phenol;
(524) 2-[6-[(4aS,8aS)-7-methyl-2,3,4,4a,5,6,8,8a-octahydro-1,7-naphthyridin-1-yl]pyridazin-3-yl]-3-methyl-5-(trifluoromethyl)phenol;
(525) 2-[6-[(4aS,8aR)-6-(2-fluoroethyl)-3,4a,5,7,8,8a-hexahydro-2H-pyrido[4,3-b][1,4]oxazin-4-yl]pyridazin-3-yl]-5-chloro-phenol;
(526) 2-[6-[(4aS,8aR)-6-ethyl-3,4a,5,7,8,8a-hexahydro-2H-pyrido[4,3-b][1,4]oxazin-4-yl]pyridazin-3-yl]-5-chloro-3-ethyl-phenol;
(527) 2-[6-[(4aS,8aR)-6-(3-fluoropropyl)-3,4a,5,7,8,8a-hexahydro-2H-pyrido[4,3-b][1,4]oxazin-4-yl]pyridazin-3-yl]-3,5-dimethyl-phenol; and
(528) 5-methyl-2-[4-methyl-6-[rac-(3aS,7aR)-6-isopropyl-3,3a,4,5,7,7a-hexahydro-2H-pyrrolo[2,3-c]pyridin-1-yl]pyridazin-3-yl]phenol.

10. (canceled)

11: A pharmaceutical composition comprising the compound of claim 1, or pharmaceutically acceptable salt thereof, and at least one pharmaceutically acceptable excipient.

12. (canceled)

13: A method of inhibiting NOD-like receptor protein 3 (NLRP3) inflammasome, the method comprising contacting the NLRP3 inflammasome with an effective amount of the compound of formula (I) according to claim 1, or a pharmaceutically acceptable salt thereof.

14: A method of preventing or treating a condition or disease in a subject in need thereof, the method comprising administering to the subject an effective amount of the compound of claim 1, or a pharmaceutically acceptable salt thereof, wherein the condition or disease is Parkinson's disease, frontotemporal Dementia, Multiple System Atrophy, Alzheimer's disease, Multiple Sclerosis, Amyotrophic Lateral Sclerosis, or brain injury.
